(19) United States
(12) Patent Application Publication
(10) Pub. No.: US 2006/0024677 A1
(43) Pub. Date: Feb. 2, 2006

(54) Novel therapeutic targets in cancer

(76) Inventors: David W. Morris, Davis, CA (US); Marc S. Malandro, Gibsonia, PA (US); Albert Lai, Davis, CA (US); Christin Tse, Libertyville, IL (US); Ali Fattaey, Oakland, CA (US)

Correspondence Address: Lisa E. Alexander;Sagres Discovery, Inc., c/o Chiron Corporation, P.O. Box 8097, Emeryville, CA, 94662-8097, US

(21) Appl. No.: 10/895,974

(22) Filed: Jul. 20, 2004

(51) Int. Cl. C12Q 1/68; C40B 50/02

(52) U.S. Cl. 435/6; 435/7.23; 702/19

(57) ABSTRACT

The present invention relates to novel sequences for use in detection, diagnosis and treatment of cancers, especially lymphomas. The invention provides cancer-associated (CA) polynucleotide sequences whose expression is associated with cancer. The present invention provides CA polypeptides associated with cancer that are present on the cell surface and present novel therapeutic targets against cancer. The present invention further provides diagnostic compositions and methods for the detection of cancer. The present invention provides monoclonal and polyclonal antibodies specific for the CA polypeptides. The present invention also provides diagnostic tools and therapeutic compositions and methods for screening, prevention and treatment of cancer.

### CROSS-REFERENCE TO RELATED APPLICATIONS

This application is related to the following U.S. Applications: U.S. Ser. No. 10/034,650, filed Dec. 20, 2001; U.S. Ser. No. 10/035,832, filed Dec. 26, 2001; U.S. Ser. No. 10/004,113, filed Oct. 23, 2001; U.S. Ser. No. 09/997,722, filed Nov. 30, 2001; U.S. Ser. No. 10/085,117, filed Feb. 27, 2002; U.S. Ser. No. 10/087,192, filed Mar. 1, 2002; U.S. Ser. No. 10/322,281, filed Dec. 17, 2002; U.S. Ser. No. 10/322,696, filed Dec. 17, 2002, U.S. Ser. No. 10/331,053, filed Dec. 26, 2002; U.S. Ser. No. 10/330,773, filed Dec. 27, 2002; U.S. Ser. No. 10/367,094, filed Feb. 14, 2003; U.S. Ser. No. 10/388,838, filed Mar. 14, 2003, U.S. Ser. No. 10/417,375 filed Apr. 15, 2003, U.S. Ser. No. 10/461,862, filed Jun. 13, 2003, U.S. Ser. No. 10/663,431 filed Sep. 15, 2003, U.S. Ser. No. 10/669,920 filed Sep. 23, 2003, U.S. Ser. No. 10/670,914 filed Sep. 24, 2003, U.S. Ser. No. 10/674,575, filed Sep. 29, 2003, U.S. Ser. No. 10/676,684 filed Sep. 30, 2003, U.S. Ser. No. 10/692,382 filed Oct. 22, 2003, U.S. Ser. No. 10/833,833, filed Apr. 27, 2004, U.S. Ser. No. 10/836,956, filed Apr. 30, 2004, and U.S. Ser. No. ______ (Atty. Docket No. 529452003100), filed Apr. 30, 2004 all of which are expressly incorporated herein by reference in their entirety.

### TECHNICAL FIELD OF THE INVENTION

This invention relates generally to the field of cancer-associated genes. Specifically, it relates to novel sequences for use in diagnosis and treatment of cancer and tumors, as well as the use of the novel compositions in screening methods. The present invention provides methods of using cancer associated polynucleotides, their corresponding gene products and antibodies specific for the gene products in the detection, diagnosis, prevention and/or treatment of associated cancers.

### BACKGROUND OF THE INVENTION

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic mutations are classified as either protooncogenes or tumor suppressor genes, depending on whether their mutant phenotypes are dominant or recessive, respectively.

There are a number of viruses known to be involved in human cancer as well as in animal cancer. Of particular interest here are viruses that do not contain oncogenes themselves; these are slow-transforming retroviruses. They induce tumors by integrating into the host genome and affecting neighboring protooncogenes in a variety of ways. Provirus insertion mutation is a normal consequence of the retroviral life cycle. In infected cells, a DNA copy of the retrovirus genome (called a provirus) is integrated into the host genome. A newly integrated provirus can affect gene expression in cis at or near the integration site by one of two mechanisms. Type I insertion mutations up-regulate transcription of proximal genes as a consequence of regulatory sequences (enhancers and/or promoters) within the proviral long terminal repeats (LTRs). Type II insertion mutations cause truncation of coding regions due to either integration directly within an open reading frame or integration within an intron flanked on both sides by coding sequences. The analysis of sequences at or near the insertion sites has led to the identification of a number of new protooncogenes.

With respect to lymphoma and leukemia, retroviruses such as AKV murine leukemia virus (MLV) or SL3-3 MLV, are potent inducers of tumors when inoculated into susceptible newborn mice, or when carried in the germline. A number of sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al., J. Virology 67:7118 (1993); Joosten et al., Virology 268:308 (2000); and Li et al., Nature Genetics 23:348 (1999); all of which are expressly incorporated by reference herein. With respect to cancers, especially breast cancer, prostate cancer and cancers with epithelial origin, the mammalian retrovirus, mouse mammary tumor virus (MMTV) is a potent inducer of tumors when inoculated into susceptible newborn mice, or when carried in the germ line. Mammary Tumors in the Mouse, edited by 3. Hilgers and M. Sluyser; Elsevier/North-Holland Biomedical Press; New York, N.Y.

The pattern of gene expression in a particular living cell is characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-326 (1991); Weinberg, Science, 254: 1138-1146 (1991)).

Accordingly, it is an object of the invention to provide polynucleotide and polypeptide sequences involved in cancer and, in particular, in oncogenesis.

Immunotherapy, or the use of antibodies for therapeutic purposes has been used in recent years to treat cancer. Passive immunotherapy involves the use of monoclonal antibodies in cancer treatments. See for example, Cancer: Principles and Practice of Oncology, 6^{th} Edition (2001) Chapt. 20 pp. 495-508. Inherent therapeutic biological activity of these antibodies include direct inhibition of tumor cell growth or survival, and the ability to recruit the natural cell killing activity of the body's immune system. These agents are administered alone or in conjunction with radiation or chemotherapeutic agents. Rituxan® and Herceptin®, approved for treatment of lymphoma and breast cancer, respectively, are two examples of such therapeutics. Alternatively, antibodies are used to make antibody conjugates where the antibody is linked to a toxic agent and directs that agent to the tumor by specifically binding to the tumor. Mylotarg® is an example of an approved antibody conjugate used for the treatment of leukemia.

Accordingly, it is another object of this invention to provide antigens (cancer-associated polypeptides) associated with a variety of cancers as targets for diagnostic and/or therapeutic antibodies. These antigens are also useful for drug discovery (e.g., small molecules) and for further characterization of cellular regulation, growth, and differentiation.

### SUMMARY OF THE INVENTION

In accordance with the objects outlined above, the present invention provides methods for screening for compositions that modulate cancer, especially lymphoma and leukemia. The present invention also provides methods for screening for compositions which modulate carcinomas, especially mammary adenocarcinomas. Also provided herein are methods of inhibiting proliferation of a cell, preferably a lymphoma cell or a breast cancer cell. Methods of treatment of cancer, including diagnosis, are also provided herein.

In one aspect, a method of screening drug candidates comprises providing a cell that expresses a cancer-associated (CA) gene or fragments thereof. Preferred embodiments of CA genes are genes that are differentially expressed in cancer cells, preferably lymphatic, breast, prostate or epithelial cells, compared to other cells. Preferred embodiments of CA genes used in the methods herein include, but are not limited to the nucleic acids selected from Tables 1-7 (human genomic sequences of SEQ ID NOS: 6, 16, 26, 40, 52, 60 and 66, and sequences of SEQ ID NOS: 7, 9, 17, 19, 21, 27, 29, 41, 43, 45, 47, 53, 61, 67, 69, 71 and 73 corresponding to the human mRNAs generated therefrom). The methods further include adding a drug candidate to the cell and determining the effect of the drug candidate on the expression of the CA gene.

In one embodiment, the method of screening drug candidates includes comparing the level of expression in the absence of the drug candidate to the level of expression in the presence of the drug candidate.

Also provided herein is a method of screening for a bioactive agent capable of binding to a CA protein (CAP), the method comprising combining the CAP and a candidate bioactive agent, and determining the binding of the candidate agent to the CAP.

Further provided herein is a method for screening for a bioactive agent capable of modulating the activity of a CAP. In one embodiment, the method comprises combining the CAP and a candidate bioactive agent, and determining the effect of the candidate agent on the bioactivity of the CAP.

Also provided is a method of evaluating the effect of a candidate cancer drug comprising administering the drug to a patient and removing a cell sample from the patient. The expression profile of the cell is then determined. This method may further comprise comparing the expression profile of the patient to an expression profile of a healthy individual.

In a further aspect, a method for inhibiting the activity of a CA protein is provided. In one embodiment, the method comprises administering to a patient an inhibitor of a CA protein preferably selected from the group consisting of the sequences outlined in Tables 1-7 (SEQ ID NOS: 8, 10, 18, 20, 22, 28, 30, 42, 44, 46, 48, 54, 62, 68, 70, 72 and 74).

A method of neutralizing the effect of a CA protein, preferably a protein encoded by a nucleic acid selected from the group of sequences outlined in Tables 1-7 (human genomic sequences of SEQ ID NOS: 6, 16, 26, 40, 52, 60 and 66, and sequences of SEQ ID NOS: 7, 9, 17, 19, 21, 27, 29, 41, 43, 45, 47, 53, 61, 67, 69, 71 and 73 corresponding to the human mRNAs generated therefrom), is also provided. Preferably, the method comprises contacting an agent specific for said protein with said protein in an amount sufficient to effect neutralization.

Moreover, provided herein is a biochip comprising a nucleic acid segment which encodes a CA protein, preferably selected from the sequences outlined in Tables 1-7 (SEQ ID NOS: 7, 9, 17, 19, 21, 27, 29, 41, 43, 45, 47, 53, 61, 67, 69, 71 and 73).

Also provided herein is a method for diagnosing or determining the propensity to cancers, especially lymphoma or leukemia or carcinoma by sequencing at least one carcinoma or lymphoma gene of an individual. In yet another aspect of the invention, a method is provided for determining cancer including lymphoma and leukemia gene copy numbers in an individual.

The invention provides an isolated nucleic acid comprising at least 10, 12, 15, 20 or 30 contiguous nucleotides of a sequence selected from the group consisting of the polynucleotide sequences SEQ ID NOS: 7, 9, 17, 19, 21, 27, 29, 41, 43, 45, 47, 53, 61, 67, 69, 71 and 73 shown in Tables 1-7, or its complement, or an expression vector comprising the isolated nucleic acids and host cells comprising them.

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

The invention provides an isolated polypeptide, encoded within an open reading frame of a CA sequence selected from the group consisting of the polynucleotide sequences of SEQ ID NOS: 6, 16, 26, 40, 52, 60 and 66 shown in Tables 1-7, or its complement. The invention provides an isolated polypeptide, wherein said polypeptide comprises the amino acid sequence encoded by a polynucleotide selected from the group consisting of SEQ ID NOS: 7, 9, 17, 19, 21, 27, 29, 41, 43, 45, 47, 53, 61, 67, 69, 71 and 73 shown in Tables 1-7. The invention provides an isolated polypeptide, wherein said polypeptide comprises the amino acid sequence encoded by a polypeptide selected from the group consisting of SEQ ID NOS: 8, 10, 18, 20, 22, 28, 30, 42, 44, 46, 48, 54, 62, 68, 70, 72 and 74 shown in Tables 1-7.

The invention further provides an isolated polypeptide, comprising the amino acid sequence of an epitope of the amino acid sequence of a CA polypeptide selected from the group consisting of SEQ ID NOS: 8, 10, 18, 20, 22, 28, 30, 42, 44, 46, 48, 54, 62, 68, 70, 72 and 74 shown in Tables 1-7, wherein the polypeptide or fragment thereof may be attached to a solid support. In one embodiment the invention provides an isolated antibody (monoclonal or polyclonal) or antigen binding fragment thereof, that binds to such a polypeptide. The isolated antibody or antigen binding fragment thereof may be attached to a solid support, or further comprises a detectable label.

In one embodiment, the invention provides a kit for diagnosing the presence of cancer in a test sample, said kit comprising at least one polynucleotide that selectively hybridizes to a CA polynucleotide sequence shown in Tables 1-7, or its complement. In another embodiment, the invention provides an electronic library comprising a CA polynucleotide, a CA polypeptide, or fragment thereof, shown in Tables 1-7.

In one embodiment, the invention provides a method of screening for anticancer activity comprising: (a) providing a cell that expresses a cancer associated (CA) gene encoded by a nucleic acid sequence selected from the group consisting of the CA sequences shown in Tables 1-7, or fragment thereof; (b) contacting a tissue sample derived from a cancer cell with an anticancer drug candidate; (c) monitoring an effect of the anticancer drug candidate on an expression of the CA polynucleotide in the tissue sample, and optionally (d) comparing the level of expression in the absence of said drug candidate to the level of expression in the presence of the drug candidate.

In one embodiment, the invention provides a method for detecting cancer associated with expression of a polypeptide in a test cell sample, comprising the steps of: (i) detecting a level of expression of at least one polypeptide selected from the group consisting of SEQ ID NOS: 8, 10, 18, 20, 22, 28, 30, 42, 44, 46, 48, 54, 62, 68, 70, 72 and 74 shown in Tables 1-7, or a fragment thereof; and (ii) comparing the level of expression of the polypeptide in the test sample with a level of expression of polypeptide in a normal cell sample, wherein an altered level of expression of the polypeptide in the test cell sample relative to the level of polypeptide expression in the normal cell sample is indicative of the presence of cancer in the test cell sample.

In another embodiment, the invention provides a method for detecting cancer associated with expression of a polypeptide in a test cell sample, comprising the steps of: (i) detecting a level of activity of at least one polypeptide selected from the group consisting of SEQ ID NOS: 8, 10, 18, 20, 22, 28, 30, 42, 44, 46, 48, 54, 62, 68, 70, 72 and 74 shown in Tables 1-7, or a fragment thereof, wherein said activity corresponds to at least one activity for the polypeptide listed in Table 9; and (ii) comparing the level of activity of the polypeptide in the test sample with a level of activity of polypeptide in a normal cell sample, wherein an altered level of activity of the polypeptide in the test cell sample relative to the level of polypeptide activity in the normal cell sample is indicative of the presence of cancer in the test cell sample.

In another embodiment, the invention provides a method for detecting cancer associated with the presence of an antibody in a test serum sample, comprising the steps of: (i) detecting a level of an antibody against an antigenic polypeptide selected from the group consisting of SEQ ID NOS: 8, 10, 18, 20, 22, 28, 30, 42, 44, 46, 48, 54, 62, 68, 70, 72 and 74 shown in Tables 1-7, or antigenic fragment thereof; and (ii) comparing said level of said antibody in the test sample with a level of said antibody in the control sample, wherein an altered level of antibody in said test sample relative to the level of antibody in the control sample is indicative of the presence of cancer in the test serum sample.

The invention provides a method for screening for a bioactive agent capable of modulating the activity of a CA protein (CAP), wherein said CAP is encoded by a nucleic acid comprising a nucleic acid sequence selected from the group consisting of the polynucleotide sequences SEQ ID NOS: 7, 9, 17, 19, 21, 27, 29, 41, 43, 45, 47, 53, 61, 67, 69, 71 and 73 shown in Tables 1-7, said method comprising: a) combining said CAP and a candidate bioactive agent; and b) determining the effect of the candidate agent on the bioactivity of said CAP. According to the method the bioactive agent: affects the expression of the CA protein (CAP); affects the activity of the CA protein (CAP), wherein such activity is selected from the activities listed in Table 9.

In one embodiment, the invention provides a method for diagnosing cancer comprising: a) determining the expression of one or more genes comprising a nucleic acid sequence selected from the group consisting of the human genomic and mRNA sequences outlined in Tables 1-7, in a first tissue type of a first individual; and b) comparing said expression of said gene(s) from a second normal tissue type from said first individual or a second unaffected individual; wherein a difference in said expression indicates that the first individual has cancer.

In another embodiment the invention provides a method for treating cancers comprising administering to a patient a bioactive agent modulating the activity of a CA protein (CAP), wherein said CAP is encoded by a nucleic acid comprising a nucleic acid sequence selected from the group consisting of the human nucleic acid sequences in Tables 1-7 and further wherein the bioactive agent binds to the CA protein, wherein the CA protein: is a signalling protein wherein the CAP sequence is selected from the group consisting of SEQ ID NOS: 6, 12, 14, 96, 130, 172 and 174 shown in Tables 1-7; is involved in signal transduction wherein the CAP sequence is selected from the group consisting of SEQ ID NOS: 20, 84, 90, 92, 94, 96, 98, 100, 102, 104, 106, and 108; is a cell adhesion protein wherein the CAP sequence is selected from the group consisting of SEQ ID NOS: 36, 38, 40, 84, 90, 92, 94, 96, 98, 100, 102, 104, 106, 108, 124, 172, and 174; is involved in inflammatory response wherein the CAP sequence is selected from the group consisting of SEQ ID NOS: 62, 64, 66, 68, 72, 74, and 76; is involved in pheromone response wherein the CAP sequence is selected from the group consisting of SEQ ID NOS: 138, 140, 142, 144, 146, 148, and 150; is an ion transport protein wherein the CAP sequence is selected from the group consisting of SEQ ID NOS: 28, 30, 114, and 118 as shown in Tables 1-7.

The invention provides monoclonal antibodies that preferentially binds to a CA protein (CAP) that is expressed on a cell surface, wherein the CA protein selected from the group consisting of SEQ ID NOS: 8, 10, 18, 20, 22, 28, 30, 42, 44, 46, 48, 54, 62, 68, 70, 72 and 74; preferably to the extracellular domain of the CA protein; preferably to a CA protein differentially expressed on a cancer cell surface relative to a normal cell surface or preferably to at least one human cancer cell line; preferably linked to a therapeutic agent; or preferably humanized. Kits and pharmaceutical compositions for detecting a presence or an absence of cancer cells in an individual, and comprising such antibodies are also provided.

The invention also provides a method for detecting a presence or an absence of cancer cells in an individual, the method comprising: contacting cells from the individual with the antibody according to the invention; and detecting a complex of a CAP from the cancer cells and the antibody, wherein detection of the complex correlates with the presence of cancer cells in the individual. In one embodiment the invention provides a method for inhibiting growth of cancer cells in an individual, the method comprising: administering to the individual an effective amount of a pharmaceutical composition according to the invention. In another embodiment the invention provides a method for delivering a therapeutic agent to cancer cells in an individual, the method comprising: administering to the individual an effective amount of a pharmaceutical composition according to according to the invention.

Novel sequences associated with cancer are also provided herein. Other aspects of the invention will become apparent to the skilled artisan by the following description of the invention.

### BRIEF DESCRIPTION OF THE FIGURES

FIG. 1 depicts PCR amplification of host-provirus junction fragments.

FIG. 2 shows an example of average threshold cycle (CT) values for a housekeeper gene and target gene.

FIG. 3 shows an example of the calculated difference (AACT) between the CT values of target and housekeeper genes (ACT) for various samples.

FIG. 4 shows the AACT and comparative expression level for each sample from FIG. 3.

### DETAILED DESCRIPTION OF PREFERRED EMBODIMENTS

The present invention is directed to a number of sequences associated with cancers, especially lymphoma, breast cancer or prostate cancer. The relatively tight linkage between clonally-integrated proviruses and protooncogenes forms “provirus tagging”, in which slow-transforming retroviruses that act by an insertion mutation mechanism are used to isolate protooncogenes. In some models, uninfected animals have low cancer rates, and infected animals have high cancer rates. It is known that many of the retroviruses involved do not carry transduced host protooncogenes or pathogenic trans-acting viral genes, and thus the cancer incidence must therefore be a direct consequence of proviral integration effects into host protooncogenes. Since proviral integration is random, rare integrants will “activate” host protooncogenes that provide a selective growth advantage, and these rare events result in new proviruses at clonal stoichiometries in tumors. In contrast to mutations caused by chemicals, radiation, or spontaneous errors, protooncogene insertion mutations can be easily located by virtue of the fact that a convenient-sized genetic marker of known sequence (the provirus) is present at the site of mutation. Host sequences that flank clonally integrated proviruses can be cloned using a variety of strategies. Once these sequences are in hand, the tagged protooncogenes can be subsequently identified. The presence of provirus at the same locus in two or more independent tumors is prima facie evidence that a protooncogene is present at or very near the provirus integration sites. This is because the genome is too large for random integrations to result in observable clustering. Any clustering that is detected is unequivocal evidence for biological selection (i.e. the tumor phenotype). Moreover, the pattern of proviral integrants (including orientations) provides compelling positional information that makes localization of the target gene at each cluster relatively simple. The three mammalian retroviruses that are known to cause cancer by an insertion mutation mechanism are FeLV (leukemia/lymphoma in cats), MLV (leukemia/lymphoma in mice and rats), and MMTV (mammary cancer in mice).

Thus, the use of oncogenic retroviruses, whose sequences insert into the genome of the host organism resulting in cancer, allows the identification of host sequences involved in cancer. These sequences may then be used in a number of different ways, including diagnosis, prognosis, screening for modulators (including both agonists and antagonists), antibody generation (for immunotherapy and imaging), etc. However, as will be appreciated by those in the art, oncogenes that are identified in one type of cancer such as lymphoma or leukemia have a strong likelihood of being involved in other types of cancers as well. Thus, while the sequences outlined herein are initially identified as correlated with lymphoma, they can also be found in other types of cancers as well, outlined below.

Definitions

Accordingly, the present invention provides nucleic acid and protein sequences that are associated with cancer, herein termed “cancer associated” or “CA” sequences. In one embodiment, the present invention provides nucleic acid and protein sequences that are associated with cancers that originate in lymphatic tissue, herein termed “lymphoma associated,” “leukemia associated” or “LA” sequences. In another embodiment, the present invention provides nucleic acid and protein sequences that are associated with carcinomas which originate in breast tissue, herein termed “breast cancer associated” or “BC” sequences.

Suitable cancers that can be diagnosed or screened for using the methods of the present invention include cancers classified by site or by histological type. Cancers classified by site include cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias).

Other cancers, classified by histological type, that may be associated with the sequences of the invention include, but are not limited to, Neoplasm, malignant; Carcinoma, NOS; Carcinoma, undifferentiated, NOS; Giant and spindle cell carcinoma; Small cell carcinoma, NOS; Papillary carcinoma, NOS; Squamous cell carcinoma, NOS; Lymphoepithelial carcinoma; Basal cell carcinoma, NOS; Pilomatrix carcinoma; Transitional cell carcinoma, NOS; Papillary transitional cell carcinoma; Adenocarcinoma, NOS; Gastrinoma, malignant; Cholangiocarcinoma; Hepatocellular carcinoma, NOS; Combined hepatocellular carcinoma and cholangiocarcinoma; Trabecular adenocarcinoma; Adenoid cystic carcinoma; Adenocarcinoma in adenomatous polyp; Adenocarcinoma, familial polyposis coli; Solid carcinoma, NOS; Carcinoid tumor, malignant; Bronchiolo-alveolar adenocarcinoma; Papillary adenocarcinoma, NOS; Chromophobe carcinoma; Acidophil carcinoma; Oxyphilic adenocarcinoma; Basophil carcinoma; Clear cell adenocarcinoma, NOS; Granular cell carcinoma; Follicular adenocarcinoma, NOS; Papillary and follicular adenocarcinoma; Nonencapsulating sclerosing carcinoma; Adrenal cortical carcinoma; Endometroid carcinoma; Skin appendage carcinoma; Apocrine adenocarcinoma; Sebaceous adenocarcinoma; Ceruminous adenocarcinoma; Mucoepidermoid carcinoma; Cystadenocarcinoma, NOS; Papillary cystadenocarcinoma, NOS; Papillary serous cystadenocarcinoma; Mucinous cystadenocarcinoma, NOS; Mucinous adenocarcinoma; Signet ring cell carcinoma; Infiltrating duct carcinoma; Medullary carcinoma, NOS; Lobular carcinoma; Inflammatory carcinoma; Paget's disease, mammary; Acinar cell carcinoma; Adenosquamous carcinoma; Adenocarcinoma w/squamous metaplasia; Thymoma, malignant; Ovarian stromal tumor, malignant; Thecoma, malignant; Granulosa cell tumor, malignant; Androblastoma, malignant; Sertoli cell carcinoma; Leydig cell tumor, malignant; Lipid cell tumor, malignant; Paraganglioma, malignant; Extra-mammary paraganglioma, malignant; Pheochromocytoma; Glomangiosarcoma; Malignant melanoma, NOS; Amelanotic melanoma; Superficial spreading melanoma; Malig melanoma in giant pigmented nevus; Epithelioid cell melanoma; Blue nevus, malignant; Sarcoma, NOS; Fibrosarcoma, NOS; Fibrous histiocytoma, malignant; Myxosarcoma; Liposarcoma, NOS; Leiomyosarcoma, NOS; Rhabdomyosarcoma, NOS; Embryonal rhabdomyosarcoma; Alveolar rhabdomyosarcoma; Stromal sarcoma, NOS; Mixed tumor, malignant, NOS; Mullerian mixed tumor; Nephroblastoma; Hepatoblastoma; Carcinosarcoma, NOS; Mesenchymoma, malignant; Brenner tumor, malignant; Phyllodes tumor, malignant; Synovial sarcoma, NOS; Mesothelioma, malignant; Dysgerminoma; Embryonal carcinoma, NOS; Teratoma, malignant, NOS; Struma ovarii, malignant; Choriocarcinoma; Mesonephroma, malignant; Hemangiosarcoma; Hemangioendothelioma, malignant; Kaposi's sarcoma; Hemangiopericytoma, malignant; Lymphangiosarcoma; Osteosarcoma, NOS; Juxtacortical osteosarcoma; Chondrosarcoma, NOS; Chondroblastoma, malignant; Mesenchymal chondrosarcoma; Giant cell tumor of bone; Ewing's sarcoma; Odontogenic tumor, malignant; Ameloblastic odontosarcoma; Ameloblastoma, malignant; Ameloblastic fibrosarcoma; Pinealoma, malignant; Chordoma; Glioma, malignant; Ependymoma, NOS; Astrocytoma, NOS; Protoplasmic astrocytoma; Fibrillary astrocytoma; Astroblastoma; Glioblastoma, NOS; Oligodendroglioma, NOS; Oligodendroblastoma; Primitive neuroectodermal; Cerebellar sarcoma, NOS; Ganglioneuroblastoma; Neuroblastoma, NOS; Retinoblastoma, NOS; Olfactory neurogenic tumor; Meningioma, malignant; Neurofibrosarcoma; Neurilemmoma, malignant; Granular cell tumor, malignant; Malignant lymphoma, NOS; Hodgkin's disease, NOS; Hodgkin's; paragranuloma, NOS; Malignant lymphoma, small lymphocytic; Malignant lymphoma, large cell, diffuse; Malignant lymphoma, follicular, NOS; Mycosis fungoides; Other specified non-Hodgkin's lymphomas; Malignant histiocytosis; Multiple myeloma; Mast cell sarcoma; Immunoproliferative small intestinal disease; Leukemia, NOS; Lymphoid leukemia, NOS; Plasma cell leukemia; Erythroleukemia; Lymphosarcoma cell leukemia; Myeloid leukemia, NOS; Basophilic leukemia; Eosinophilic leukemia; Monocytic leukemia, NOS; Mast cell leukemia; Megakaryoblastic leukemia; Myeloid sarcoma; and Hairy cell leukemia.

In addition, the CA genes may be involved in other diseases such as, but not limited to, diseases associated with aging or neurodegeneration.

“Association” in this context means that the nucleotide or protein sequences are either differentially expressed, activated, inactivated or altered in cancers as compared to normal tissue. As outlined below, CA sequences include those that are up-regulated (i.e. expressed at a higher level), as well as those that are down-regulated (i.e. expressed at a lower level), in cancers. CA sequences also include sequences that have been altered (i.e., truncated sequences or sequences with substitutions, deletions or insertions, including point mutations) and show either the same expression profile or an altered profile. In a preferred embodiment, the CA sequences are from humans; however, as will be appreciated by those in the art, CA sequences from other organisms may be useful in animal models of disease and drug evaluation; thus, other CA sequences are provided, from vertebrates, including mammals, including rodents (rats, mice, hamsters, guinea pigs, etc.), primates, and farm animals (including sheep, goats, pigs, cows, horses, etc). In some cases, prokaryotic CA sequences may be useful. CA sequences from other organisms may be obtained using the techniques outlined below.

CA sequences include both nucleic acid and amino acid sequences. In a preferred embodiment, the CA sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus a recombinant nucleic acid is also an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it will replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein a “polynucleotide” or “nucleic acid” is a polymeric form of nucleotides of any length, either ribonucleotides or deoxyribonucleotides. This term refers only to the primary structure of the molecule. Thus, this term includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a CA gene.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75% by weight of the total protein, with about 80% being preferred, and about 90% being particularly preferred. The definition includes the production of a CA protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the CA sequences are nucleic acids. As will be appreciated by those in the art and is more fully outlined below, CA sequences are useful in a variety of applications, including diagnostic applications, which will detect naturally occurring nucleic acids, as well as screening applications; for example, biochips comprising nucleic acid probes to the CA sequences can be generated. In the broadest sense, use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. A “polynucleotide” or “oligonucleotide” may comprise DNA, RNA, PNA or a polymer of nucleotides linked by phosphodiester and/or any alternate bonds.

A nucleic acid of the present invention generally contains phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc; 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. As will be appreciated by those in the art, the depiction of a single strand “Watson” also defines the sequence of the other strand “Crick”; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to amplify or clone target polynucleotides. Since the position of each particular group of primers in the array is known, the identities of the target polynucleotides can be determined based on their binding to a particular position in the microarray.

A “linker” is a synthetic oligodeoxyribonucleotide that contains a restriction site. A linker may be blunt end-ligated onto the ends of DNA fragments to create restriction sites that can be used in the subsequent cloning of the fragment into a vector molecule.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical, chemical, or any other appropriate means. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

The term “support” refers to conventional supports such as beads, particles, dipsticks, fibers, filters, membranes, and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases.

As used herein, a “biological sample” refers to a sample of tissue or fluid isolated from an individual, including but not limited to, for example, blood, plasma, serum, spinal fluid, lymph fluid, skin, respiratory, intestinal and genitourinary tracts, tears, saliva, milk, cells (including but not limited to blood cells), tumors, organs, and also samples of in vitro cell culture constituents.

The term “biological sources” as used herein refers to the sources from which the target polynucleotides are derived. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

Cancer-Associated Sequences

The CA sequences of the invention were initially identified by infection of mice with a retrovirus such as murine leukemia virus (MLV) resulting in lymphoma. Retroviruses have a genome that is made out of RNA. After a retrovirus infects a host cell, a double stranded DNA copy of the retrovirus genome (a “provirus”) is inserted into the genomic DNA of the host cell. The integrated provirus may affect the expression of host genes at or near the site of integration—a phenomenon known as retroviral insertional mutagenesis. Possible changes in the expression of host cell genes include: (i) increased expression of genes near the site of integration resulting from the proximity of elements in the provirus that act as transcriptional promoters and enhancers, (ii) functional inactivation of a gene caused by the integration of a provirus into the gene itself thus preventing the synthesis of a functional gene product, or (iii) expression of a mutated protein that has a different activity to the normal protein. Typically such a protein would be prematurely truncated and lack a regulatory domain near the C terminus. Such a protein might be constitutively active, or act as a dominant negative inhibitor of the normal protein. For example, retrovirus enhancers, including that of SL3-3, are known to act on genes up to approximately 200 kilobases from the insertion site. Moreover, many of these sequences are also involved in other cancers and disease states. Sequences of mouse genes according to this invention, that are identified in this manner are shown as mDxx-yyy in Tables 1-7.

A CA sequence can be initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

In one embodiment, CA sequences are those that are up-regulated in cancers; that is, the expression of these genes is higher in cancer tissue as compared to normal tissue of the same differentiation stage. “Up-regulation” as used herein means increased expression by about 50%, preferably about 100%, more preferably about 150% to about 200%, with up-regulation from 300% to 1000% being preferred.

In another embodiment, CA sequences are those that are down-regulated in cancers; that is, the expression of these genes is lower in cancer tissue as compared to normal tissue of the same differentiation stage. “Down-regulation” as used herein means decreased expression by about 50%, preferably about 100%, more preferably about 150% to about 200%, with down-regulation from 300% to 1000% to no expression being preferred.

In yet another embodiment, CA sequences are those that have altered sequences but show either the same or an altered expression profile as compared to normal lymphoid tissue of the same differentiation stage. “Altered CA sequences” as used herein also refers to sequences that are truncated, contain insertions or contain point mutations.

CA proteins of the present invention may be classified as secreted proteins, transmembrane proteins or intracellular proteins. In a preferred embodiment the CA protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

In a preferred embodiment, the CA sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors are classified as “seven transmembrane domain” proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, leptin receptor, interleukin receptors, e.g. IL-1 receptor, IL-2 receptor, etc. CA proteins may be derived from genes that regulate apoptosis (IL-3, GM-CSF and Bcl-x) or are shown to have a role in the regulation of apoptosis.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell for example via a glycosylphosphatidylinositol (GPD) anchor, or may themselves be transmembrane proteins. Extracellular domains also associate with the extracellular matrix and contribute to the maintenance of the cell structure.

CA proteins that are transmembrane are particularly preferred in the present invention as they are good targets for immunotherapeutics, as are described herein. In addition, as outlined below, transmembrane proteins can be also useful in imaging modalities.

It will also be appreciated by those in the art that a transmembrane protein can be made soluble by removing transmembrane sequences, for example through recombinant methods. Furthermore, transmembrane proteins that have been made soluble can be made to be secreted through recombinant means by adding an appropriate signal sequence.

In a preferred embodiment, the CA proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. CA proteins that are secreted proteins are particularly preferred in the present invention as they serve as good targets for diagnostic markers, for example for blood tests.

CA Sequences and Homologs

A CA sequence is initially identified by substantial nucleic acid and/or amino acid sequence homology to the CA sequences outlined herein. Such homology can be based upon the overall nucleic acid or amino acid sequence, and is generally determined as outlined below, using either homology programs or hybridization conditions.

As used herein, a nucleic acid is a “CA nucleic acid” if the overall homology of the nucleic acid sequence to one of the nucleic acids of Tables 1-7 is preferably greater than about 75%, more preferably greater than about 80%, even more preferably greater than about 85% and most preferably greater than 90%. In some embodiments the homology will be as high as about 93 to 95 or 98%. In a preferred embodiment, the sequences that are used to determine sequence identity or similarity are selected from those of the nucleic acids of Tables 1-7. In another embodiment, the sequences are naturally occurring allelic variants of the sequences of the nucleic acids of Tables 1-7. In another embodiment, the sequences are sequence variants as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters include a default gap weight of 3.00, a default gap length weight of 0.10, and weighted end gaps.

Another example of a useful algorithm is the BLAST (Basic Local Alignment Search Tool) algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl.edu/]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic acids of Tables 1-7. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acids of Tables 1-7, it is understood that the percentage of homology will be determined based on the number of homologous nucleosides in relation to the total number of nucleosides. Thus homology of sequences shorter than those of the sequences identified herein will be determined using the number of nucleosides in the shorter sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC (“saline sodium citrate”; 9 mM NaCl, 0.9 mM sodium citrate), 0.5% SDS, 1.0 mM EDTA (pH 8.0); hybridizing at 50-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C., or 65-70° C. Stringent conditions may also be achieved with the addition of destabilizing agents such as formamide.

Thus nucleic acids that hybridize under high stringency to the nucleic acids identified in the figures, or their complements, are considered CA sequences. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for longer probes (e.g. greater than 50 nucleotides). In another embodiment, less stringent hybridization conditions are used; for example, moderate or low stringency conditions may be used, as are known in the art; see Maniatis and Ausubel, supra, and Tijssen, supra.

In addition, the CA nucleic acid sequences of the invention are fragments of larger genes, i.e. they are nucleic acid segments. Alternatively, the CA nucleic acid sequences can serve as indicators of oncogene position, for example, the CA sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the CA genes can be obtained, using techniques well known in the art for cloning either longer sequences or the full-length sequences; see Maniatis et al., and Ausubel, et al., supra, hereby expressly incorporated by reference. In general, this is done using PCR, for example, kinetic PCR.

Detection of CA Expression

Once the CA nucleic acid is identified, it can be cloned and, if necessary, its constituent parts recombined to form the entire CA nucleic acid. Once isolated from its natural source, e.g., contained within a plasmid or other vector or excised therefrom as a linear nucleic acid segment, the recombinant CA nucleic acid can be further used as a probe to identify and isolate other CA nucleic acids, for example additional coding regions. It can also be used as a “precursor” nucleic acid to make modified or variant CA nucleic acids and proteins. In a preferred embodiment, once a CA gene is identified its nucleotide sequence is used to design probes specific for the CA gene.

The CA nucleic acids of the present invention are used in several ways. In a first embodiment, nucleic acid probes hybridizable to CA nucleic acids are made and attached to biochips to be used in screening and diagnostic methods, or for gene therapy and/or antisense applications. Alternatively, the CA nucleic acids that include coding regions of CA proteins can be put into expression vectors for the expression of CA proteins, again either for screening purposes or for administration to a patient.

Recent developments in DNA microarray technology make it possible to conduct a large scale assay of a plurality of target CA nucleic acid molecules on a single solid phase support. U.S. Pat. No. 5,837,832 (Chee et al.) and related patent applications describe immobilizing an array of oligonucleotide probes for hybridization and detection of specific nucleic acid sequences in a sample. Target polynucleotides of interest isolated from a tissue of interest are hybridized to the DNA chip and the specific sequences detected based on the target polynucleotides' preference and degree of hybridization at discrete probe locations. One important use of arrays is in the analysis of differential gene expression, where the profile of expression of genes in different cells, often a cell of interest and a control cell, is compared and any differences in gene expression among the respective cells are identified. Such information is useful for the identification of the types of genes expressed in a particular cell or tissue type and diagnosis of cancer conditions based on the expression profile.

Typically, RNA from the sample of interest is subjected to reverse transcription to obtain labeled cDNA. See U.S. Pat. No. 6,410,229 (Lockhart et al.) The cDNA is then hybridized to oligonucleotides or cDNAs of known sequence arrayed on a chip or other surface in a known order. The location of the oligonucleotide to which the labeled cDNA hybridizes provides sequence information on the cDNA, while the amount of labeled hybridized RNA or cDNA provides an estimate of the relative representation of the RNA or cDNA of interest. See Schena, et al. Science 270:467-470 (1995). For example, use of a cDNA microarray to analyze gene expression patterns in human cancer is described by DeRisi, et al. (Nature Genetics 14:457-460 (1996)).

In a preferred embodiment, nucleic acid probes corresponding to CA nucleic acids (both the nucleic acid sequences outlined in the figures and/or the complements thereof) are made. Typically, these probes are synthesized based on the disclosed sequences of this invention. The nucleic acid probes attached to the biochip are designed to be substantially complementary to the CA nucleic acids, i.e. the target sequence (either the target sequence of the sample or to other probe sequences, for example in sandwich assays), such that specific hybridization of the target sequence and the probes of the present invention occurs. As outlined below, this complementarity need not be perfect, in that there may be any number of base pair mismatches that will interfere with hybridization between the target sequence and the single stranded nucleic acids of the present invention. It is expected that the overall homology of the genes at the nucleotide level probably will be about 40% or greater, probably about 60% or greater, and even more probably about 80% or greater; and in addition that there will be corresponding contiguous sequences of about 8-12 nucleotides or longer. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein. Whether or not a sequence is unique to a CA gene according to this invention can be determined by techniques known to those of skill in the art. For example, the sequence can be compared to sequences in databanks, e.g., GeneBank, to determine whether it is present in the uninfected host or other organisms. The sequence can also be compared to the known sequences of other viral agents, including those that are known to induce cancer.

A nucleic acid probe is generally single stranded but can be partly single and partly double stranded. The strandedness of the probe is dictated by the structure, composition, and properties of the target sequence. In general, the oligonucleotide probes range from about 6, 8, 10, 12, 15, 20, 30 to about 100 bases long, with from about 10 to about 80 bases being preferred, and from about 30 to about 50 bases being particularly preferred. That is, generally entire genes are rarely used as probes. In some embodiments, much longer nucleic acids can be used, up to hundreds of bases. The probes are sufficiently specific to hybridize to complementary template sequence under conditions known by those of skill in the art. The number of mismatches between the probes sequences and their complementary template (target) sequences to which they hybridize during hybridization generally do not exceed 15%, usually do not exceed 10% and preferably do not exceed 5%, as determined by FASTA (default settings).

Oligonucleotide probes can include the naturally-occurring heterocyclic bases normally found in nucleic acids (uracil, cytosine, thymine, adenine and guanine), as well as modified bases and base analogues. Any modified base or base analogue compatible with hybridization of the probe to a target sequence is useful in the practice of the invention. The sugar or glycoside portion of the probe can comprise deoxyribose, ribose, and/or modified forms of these sugars, such as, for example, 2′-O-alkyl ribose. In a preferred embodiment, the sugar moiety is 2′-deoxyribose; however, any sugar moiety that is compatible with the ability of the probe to hybridize to a target sequence can be used.

In one embodiment, the nucleoside units of the probe are linked by a phosphodiester backbone, as is well known in the art. In additional embodiments, internucleotide linkages can include any linkage known to one of skill in the art that is compatible with specific hybridization of the probe including, but not limited to phosphorothioate, methylphosphonate, sulfamate (e.g., U.S. Pat. No. 5,470,967) and polyamide (i.e., peptide nucleic acids). Peptide nucleic acids are described in Nielsen et al. (1991) Science 254: 1497-1500, U.S. Pat. No. 5,714,331, and Nielsen (1999) Curr. Opin. Biotechnol. 10:71-75.

In certain embodiments, the probe can be a chimeric molecule; i.e., can comprise more than one type of base or sugar subunit, and/or the linkages can be of more than one type within the same primer. The probe can comprise a moiety to facilitate hybridization to its target sequence, as are known in the art, for example, intercalators and/or minor groove binders. Variations of the bases, sugars, and internucleoside backbone, as well as the presence of any pendant group on the probe, will be compatible with the ability of the probe to bind, in a sequence-specific fashion, with its target sequence. A large number of structural modifications, both known and to be developed, are possible within these bounds. Advantageously, the probes according to the present invention may have structural characteristics such that they allow the signal amplification, such structural characteristics being, for example, branched DNA probes as those described by Urdea et al. (Nucleic Acids Symp. Ser., 24:197-200 (1991)) or in the European Patent No. EP-0225,807. Moreover, synthetic methods for preparing the various heterocyclic bases, sugars, nucleosides and nucleotides that form the probe, and preparation of oligonucleotides of specific predetermined sequence, are well-developed and known in the art. A preferred method for oligonucleotide synthesis incorporates the teaching of U.S. Pat. No. 5,419,966.

Multiple probes may be designed for a particular target nucleic acid to account for polymorphism and/or secondary structure in the target nucleic acid, redundancy of data and the like. In some embodiments, where more than one probe per sequence is used, either overlapping probes or probes to different sections of a single target CA gene are used. That is, two, three, four or more probes, with three being preferred, are used to build in a redundancy for a particular target. The probes can be overlapping (i.e. have some sequence in common), or specific for distinct sequences of a CA gene. When multiple target polynucleotides are to be detected according to the present invention, each probe or probe group corresponding to a particular target polynucleotide is situated in a discrete area of the microarray.

Probes may be in solution, such as in wells or on the surface of a micro-array, or attached to a solid support. Examples of solid support materials that can be used include a plastic, a ceramic, a metal, a resin, a gel and a membrane. Useful types of solid supports include plates, beads, magnetic material, microbeads, hybridization chips, membranes, crystals, ceramics and self-assembling monolayers. A preferred embodiment comprises a two-dimensional or three-dimensional matrix, such as a gel or hybridization chip with multiple probe binding sites (Pevzner et al., J. Biomol. Struc. & Dyn. 9:399-410, 1991; Maskos and Southern, Nuc. Acids Res. 20:1679-84, 1992). Hybridization chips can be used to construct very large probe arrays that are subsequently hybridized with a target nucleic acid. Analysis of the hybridization pattern of the chip can assist in the identification of the target nucleotide sequence. Patterns can be manually or computer analyzed, but it is clear that positional sequencing by hybridization lends itself to computer analysis and automation. Algorithms and software, which have been developed for sequence reconstruction, are applicable to the methods described herein (R. Drmanac et al., J. Biomol. Struc. & Dyn. 5:1085-1102, 1991; P. A. Pevzner, J. Biomol. Struc. & Dyn. 7:63-73, 1989).

As will be appreciated by those in the art, nucleic acids can be attached or immobilized to a solid support in a wide variety of ways. By “immobilized” herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as streptavidin, to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

Nucleic acid probes may be attached to the solid support by covalent binding such as by conjugation with a coupling agent or by, covalent or non-covalent binding such as electrostatic interactions, hydrogen bonds or antibody-antigen coupling, or by combinations thereof. Typical coupling agents include biotin/avidin, biotin/streptavidin, Staphylococcus aureus protein A/IgG antibody F, fragment, and streptavidin/protein A chimeras (T. Sano and C. R. Cantor, Bio/Technology 9:1378-81 (1991)), or derivatives or combinations of these agents. Nucleic acids may be attached to the solid support by a photocleavable bond, an electrostatic bond, a disulfide bond, a peptide bond, a diester bond or a combination of these sorts of bonds. The array may also be attached to the solid support by a selectively releasable bond such as 4,4′-dimethoxytrityl or its derivative. Derivatives which have been found to be useful include 3 or 4 [bis-(4-methoxyphenyl)]-methyl-benzoic acid, N-succinimidyl-3 or 4 [bis-(4-methoxyphenyl)]-methyl-benzoic acid, N-succinimidyl-3 or 4 [bis-(4-methoxyphenyl)]-hydroxymethyl-benzoic acid, N-succinimidyl-3 or 4 [bis-(4-methoxyphenyl)]-chloromethyl-benzoic acid, and salts of these acids.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. The solid phase support of the present invention can be of any solid materials and structures suitable for supporting nucleotide hybridization and synthesis. Preferably, the solid phase support comprises at least one substantially rigid surface on which the primers can be immobilized and the reverse transcriptase reaction performed. The substrates with which the polynucleotide microarray elements are stably associated may be fabricated from a variety of materials, including plastics, ceramics, metals, acrylamide, cellulose, nitrocellulose, glass, polystyrene, polyethylene vinyl acetate, polypropylene, polymethacrylate, polyethylene, polyethylene oxide, polysilicates, polycarbonates, Teflon®, fluorocarbons, nylon, silicon rubber, polyanhydrides, polyglycolic acid, polylactic acid, polyorthoesters, polypropylfumerate, collagen, glycosaminoglycans, and polyamino acids. Substrates may be two-dimensional or three-dimensional in form, such as gels, membranes, thin films, glasses, plates, cylinders, beads, magnetic beads, optical fibers, woven fibers, etc. A preferred form of array is a three-dimensional array. A preferred three-dimensional array is a collection of tagged beads. Each tagged bead has different primers attached to it. Tags are detectable by signaling means such as color (Luminex, Illumina) and electromagnetic field (Pharmaseq) and signals on tagged beads can even be remotely detected (e.g., using optical fibers). The size of the solid support can be any of the standard microarray sizes, useful for DNA microarray technology, and the size may be tailored to fit the particular machine being used to conduct a reaction of the invention. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo- or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside. In an additional embodiment, the immobilization to the solid support may be very strong, yet non-covalent. For example, biotinylated oligonucleotides can be made, which bind to surfaces covalently coated with streptavidin, resulting in attachment.

The arrays may be produced according to any convenient methodology, such as preforming the polynucleotide microarray elements and then stably associating them with the surface. Alternatively, the oligonucleotides may be synthesized on the surface, as is known in the art. A number of different array configurations and methods for their production are known to those of skill in the art and disclosed in WO 95/25116 and WO 95/35505 (photolithographic techniques), U.S. Pat. No. 5,445,934 (in situ synthesis by photolithography), U.S. Pat. No. 5,384,261 (in situ synthesis by mechanically directed flow paths); and U.S. Pat. No. 5,700,637 (synthesis by spotting, printing or coupling); the disclosure of which are herein incorporated in their entirety by reference. Another method for coupling DNA to beads uses specific ligands attached to the end of the DNA to link to ligand-binding molecules attached to a bead. Possible ligand-binding partner pairs include biotin-avidin/streptavidin, or various antibody/antigen pairs such as digoxygenin-antidigoxygenin antibody (Smith et al., “Direct Mechanical Measurements of the Elasticity of Single DNA Molecules by Using Magnetic Beads,” Science 258:1122-1126 (1992)). Covalent chemical attachment of DNA to the support can be accomplished by using standard coupling agents to link the 5′-phosphate on the DNA to coated microspheres through a phosphoamidate bond. Methods for immobilization of oligonucleotides to solid-state substrates are well established. See Pease et al., Proc. Natl. Acad. Sci. USA 91(11):5022-5026 (1994). A preferred method of attaching oligonucleotides to solid-state substrates is described by Guo et al., Nucleic Acids Res. 22:5456-5465 (1994). Immobilization can be accomplished either by in situ DNA synthesis (Maskos and Southern, Nucleic Acids Research, 20:1679-1684 (1992) or by covalent attachment of chemically synthesized oligonucleotides (Guo et al., supra) in combination with robotic arraying technologies.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMang technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, an array of probe/primer reactions can simultaneously quantify the expression of multiple gene products of interest. See Germer, S., et al., Genome Res. 10:258-266 (2000); Heid, C. A., et al., Genome Res. 6, 986-994 (1996).

Expression of CA Proteins

In a preferred embodiment, CA nucleic acids encoding CA proteins are used to make a variety of expression vectors to express CA proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the CA protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the CA protein; for example, transcriptional and translational regulatory nucleic acid sequences from Bacillus are preferably used to express the CA protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences that flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The CA proteins of the present invention are produced by culturing a host cell transformed with an expression vector containing nucleic acid encoding a CA protein, under the appropriate conditions to induce or cause expression of the CA protein. The conditions appropriate for CA protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the CA proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived form SV40.

The methods of introducing exogenous nucleic acid into mammalian hosts, as well as other hosts, are well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, CA proteins are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the CA protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes that render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, CA proteins are produced in insect cells. Expression vectors for the transformation of insect cells, and in particular, baculovirus-based expression vectors, are well known in the art.

In a preferred embodiment, CA protein is produced in yeast cells. Yeast expression systems are well known in the art, and include expression vectors for Saccharomyces cerevisiae, Candida albicans and C. maltosa, Hansenula polymorpha, Kluyveromyces fragilis and K lactis, Pichia guillerimondii and P. pastoris, Schizosaccharomyces pombe, and Yarrowia lipolytica.

The CA protein may also be made as a fusion protein, using techniques well known in the art. Thus, for example, for the creation of monoclonal antibodies. If the desired epitope is small, the CA protein may be fused to a carrier protein to form an immunogen. Alternatively, the CA protein may be made as a fusion protein to increase expression, or for other reasons. For example, when the CA protein is a CA peptide, the nucleic acid encoding the peptide may be linked to other nucleic acid for expression purposes.

In one embodiment, the CA nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the CA nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides CA protein sequences. A CA protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the CA protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “query Genetic Codes” is standard (1). The matrix is BLOSUM 62; gap existence cost is 11, per residue gap cost is 1; and the lambda ratio is 0.85 default. This results in the generation of a putative protein sequence.

In general, the term “polypeptide” as used herein refers to both the full-length polypeptide encoded by the recited polynucleotide, the polypeptide encoded by the gene represented by the recited polynucleotide, as well as portions or fragments thereof. The present invention encompasses variants of the naturally occurring proteins, wherein such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, at least about 81%, at least about 82%, at least about 83%, at least about 84%, at least about 85%, at least about 86%, at least about 87%, at least about 88%, at least about 89%, usually at least about 90%, at least 91%, at least 92%, at least 93%, at least 94%, at least 95%, at least 96%, at least 97%, at least 98% and more usually at least about 99% sequence identity with a differentially expressed polypeptide described herein, as determined by the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2: 482-489. The variant polypeptides can be naturally or non-naturally glycosylated, i.e., the polypeptide has a glycosylation pattern that differs from the glycosylation pattern found in the corresponding naturally occurring protein.

Also within the scope of the invention are variants. Variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions within proline loops (see, e.g., Masul et al., Appl. Env. Microbiol. (1994) 60:3579). Cysteine-depleted muteins can be produced as disclosed in U.S. Pat. No. 4,959,314.

Variants also include fragments of the polypeptides disclosed herein, particularly biologically active fragments and/or fragments corresponding to functional domains. Fragments of interest will typically be at least about 8 amino acids (aa) 10 aa, 15 aa, 20 aa, 25 aa, 30 aa, 35 aa, 40 aa, to at least about 45 aa in length, usually at least about 50 aa in length, at least about 75 aa, at least about 100 aa, at least about 125 aa, at least about 150 aa in length, at least about 200 aa, at least about 300 aa, at least about 400 aa and can be as long as 500 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. The protein variants described herein are encoded by polynucleotides that are within the scope of the invention. The genetic code can be used to select the appropriate codons to construct the corresponding variants.

While altered expression of the polynucleotides associated with cancer is observed, altered levels of expression of the polypeptides encoded by these polynucleotides may likely play a role in cancers.

Also included within one embodiment of CA proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. The present application is also directed to proteins containing polypeptides at least 80%, 85%, 90%, 95%, 96%, 97%, 98% or 99% identical to a CA polypeptide sequence set forth herein. As for nucleic acids, homology in this context means sequence similarity or identity, with identity being preferred. This homology will be determined using standard techniques known in the art as are outlined above for the nucleic acid homologies.

CA proteins of the present invention may be shorter or longer than the wild type amino acid sequences. Thus, in a preferred embodiment, included within the definition of CA proteins are portions or fragments of the wild type sequences herein. In addition, as outlined above, the CA nucleic acids of the invention may be used to obtain additional coding regions, and thus additional protein sequence, using techniques known in the art.

In a preferred embodiment, the CA proteins are derivative or variant CA proteins as compared to the wild-type sequence. That is, as outlined more fully below, the derivative CA peptide will contain at least one amino acid substitution, deletion or insertion, with amino acid substitutions being particularly preferred. The amino acid substitution, insertion or deletion may occur at any residue within the CA peptide.

Also included in an embodiment of CA proteins of the present invention are amino acid sequence variants. These variants fall into one or more of three classes: substitutional, insertional or deletional variants. These variants ordinarily are prepared by site-specific mutagenesis of nucleotides in the DNA encoding the CA protein, using cassette or PCR mutagenesis or other techniques well known in the art, to produce DNA encoding the variant, and thereafter expressing the DNA in recombinant cell culture as outlined above. However, variant CA protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the CA protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed CA variants screened for the optimal combination of desired activity. Techniques for making substitution mutations at predetermined sites in DNA having a known sequence are well known, for example, M13 primer mutagenesis and LAR mutagenesis. Screening of the mutants is done using assays of CA protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the CA protein are desired, substitutions are generally made in accordance with the following chart:

CHART 1

| Original | Exemplary |
|---|---|
| Residue | Substitutions |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made full length to more significantly affect one or more of the following: the structure of the polypeptide backbone in the area of the alteration (e.g., the alpha-helical or beta-sheet structure); the charge or hydrophobicity of the molecule at the target site; and the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the CA proteins as needed. Alternatively, the variant may be designed such that the biological activity of the CA protein is altered. For example, glycosylation sites may be altered or removed, dominant negative mutations created, etc.

Covalent modifications of CA polypeptides are included within the scope of this invention, for example for use in screening. One type of covalent modification includes reacting targeted amino acid residues of a CA polypeptide with an organic derivatizing agent that is capable of reacting with selected side chains or the N- or C-terminal residues of a CA polypeptide. Derivatization with bifunctional agents is useful, for instance, for crosslinking CA polypeptides to a water-insoluble support matrix or surface for use in the method for purifying anti-CA antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the a-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the CA polypeptide included within the scope of this invention comprises altering the native glycosylation pattern of the polypeptide. “Altering the native glycosylation pattern” is intended for purposes herein to mean deleting one or more carbohydrate moieties found in native sequence CA polypeptide, and/or adding one or more glycosylation sites that are not present in the native sequence CA polypeptide.

Addition of glycosylation sites to CA polypeptides may be accomplished by altering the amino acid sequence thereof. The alteration may be made, for example, by the addition of, or substitution by, one or more serine or threonine residues to the native sequence CA polypeptide (for O-linked glycosylation sites). The CA amino acid sequence may optionally be altered through changes at the DNA level, particularly by mutating the DNA encoding the CA polypeptide at preselected bases such that codons are generated that will translate into the desired amino acids.

Another means of increasing the number of carbohydrate moieties on the CA polypeptide is by chemical or enzymatic coupling of glycosides to the polypeptide. Such methods are described in the art, e.g., in WO 87/05330 published 11 Sep. 1987, and in Aplin and Wriston, L A Crit. Rev. Biochem., pp. 259-306 (1981).

Removal of carbohydrate moieties present on the CA polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of CA comprises linking the CA polypeptide to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

CA polypeptides of the present invention may also be modified in a way to form chimeric molecules comprising a CA polypeptide fused to another, heterologous polypeptide or amino acid sequence. In one embodiment, such a chimeric molecule comprises a fusion of a CA polypeptide with a tag polypeptide that provides an epitope to which an anti-tag antibody can selectively bind. The epitope tag is generally placed at the amino- or carboxyl-terminus of the CA polypeptide, although internal fusions may also be tolerated in some instances. The presence of such epitope-tagged forms of a CA polypeptide can be detected using an antibody against the tag polypeptide. Also, provision of the epitope tag enables the CA polypeptide to be readily purified by affinity purification using an anti-tag antibody or another type of affinity matrix that binds to the epitope tag. In an alternative embodiment, the chimeric molecule may comprise a fusion of a CA polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of CA protein in one embodiment are other CA proteins of the CA family, and CA proteins from other organisms, which are cloned and expressed as outlined below. Thus, probe or degenerate polymerase chain reaction (PCR) primer sequences may be used to find other related CA proteins from humans or other organisms. As will be appreciated by those in the art, particularly useful probe and/or PCR primer sequences include the unique areas of the CA nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, CA proteins can be made that are longer than those encoded by the nucleic acids of the figures, for example, by the elucidation of additional sequences, the addition of epitope or purification tags, the addition of other fusion sequences, etc.

CA proteins may also be identified as being encoded by CA nucleic acids. Thus, CA proteins are encoded by nucleic acids that will hybridize to the sequences of the sequence listings, or their complements, as outlined herein.

CA Antigens and Antibodies Thereto

In one embodiment, the invention provides CA specific antibodies. In a preferred embodiment, when the CA protein is to be used to generate antibodies, for example for immunotherapy, the CA protein should share at least one epitope or determinant with the full-length protein. By “epitope” or “determinant” herein is meant a portion of a protein that will generate and/or bind an antibody or T-cell receptor in the context of MHC. Thus, in most instances, antibodies made to a smaller CA protein will be able to bind to the full-length protein. In a preferred embodiment, the epitope is unique; that is, antibodies generated to a unique epitope show little or no cross-reactivity.

Any polypeptide sequence encoded by the CA polynucleotide sequences may be analyzed to determine certain preferred regions of the polypeptide. Regions of high antigenicity are determined from data by DNASTAR analysis by choosing values that represent regions of the polypeptide that are likely to be exposed on the surface of the polypeptide in an environment in which antigen recognition may occur in the process of initiation of an immune response. For example, the amino acid sequence of a polypeptide encoded by a CA polynucleotide sequence may be analyzed using the default parameters of the DNASTAR computer algorithm (DNASTAR, Inc., Madison, Wis.; http://www.dnastar.com/).

Polypeptide features that may be routinely obtained using the DNASTAR computer algorithm include, but are not limited to, Garnier-Robson alpha-regions, beta-regions, turn-regions, and coil-regions (Garnier et al. J. Mol. Biol., 120: 97 (1978)); Chou-Fasman alpha-regions, beta-regions, and turn-regions (Adv. in Enzymol., 47:45-148 (1978)); Kyte-Doolittle hydrophilic regions and hydrophobic regions (J. Mol. Biol., 157:105-132 (1982)); Eisenberg alpha- and beta-amphipathic regions; Karplus-Schulz flexible regions; Emini surface-forming regions (J. Virol., 55(3):836-839 (1985)); and Jameson-Wolf regions of high antigenic index (CABIOS, 4(1):181-186 (1988)). Kyte-Doolittle hydrophilic regions and hydrophobic regions, Emini surface-forming regions, and Jameson-Wolf regions of high antigenic index (i.e., containing four or more contiguous amino acids having an antigenic index of greater than or equal to 1.5, as identified using the default parameters of the Jameson-Wolf program) can routinely be used to determine polypeptide regions that exhibit a high degree of potential for antigenicity. One approach for preparing antibodies to a protein is the selection and preparation of an amino acid sequence of all or part of the protein, chemically synthesizing the sequence and injecting it into an appropriate animal, typically a rabbit, hamster or a mouse. Oligopeptides can be selected as candidates for the production of an antibody to the CA protein based upon the oligopeptides lying in hydrophilic regions, which are thus likely to be exposed in the mature protein. Additional oligopeptides can be determined using, for example, the Antigenicity Index, Welling, G. W. et al., FEBS Lett. 188:215-218 (1985), incorporated herein by reference.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-7, or fragment thereof or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Monoclonal antibody technology is used in implementing research, diagnosis and therapy. Monoclonal antibodies are used in radioimmunoassays, enzyme-linked immunosorbent assays, immunocytopathology, and flow cytometry for in vitro diagnosis, and in vivo for diagnosis and immunotherapy of human disease. Waldmann, T. A. (1991) Science 252:1657-1662. In particular, monoclonal antibodies have been widely applied to the diagnosis and therapy of cancer, wherein it is desirable to target malignant lesions while avoiding normal tissue. See, e.g., U.S. Pat. No. 4,753,894 to Frankel, et al.; U.S. Pat. No. 4,938,948 to Ring et al.; and U.S. Pat. No. 4,956,453 to Bjorn et al.

In one embodiment, the antibodies are bispecific antibodies. Bispecific antibodies are monoclonal, preferably human or humanized, antibodies that have binding specificities for at least two different antigens. A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients. In the present case, one of the binding specificities is for a protein encoded by a nucleic acid of Tables 1-7, or a fragment thereof, the other one is for any other antigen, and preferably for a cell-surface protein or receptor or receptor subunit, preferably one that is tumor specific.

In a preferred embodiment, the antibodies to CA are capable of reducing or eliminating the biological function of CA, as is described below. That is, the addition of anti-CA antibodies (either polyclonal or preferably monoclonal) to CA (or cells containing CA) may reduce or eliminate the CA activity. Generally, at least a 25% decrease in activity is preferred, with at least about 50% being particularly preferred and about a 95-100% decrease being especially preferred.

In a preferred embodiment the antibodies to the CA proteins are humanized antibodies. “Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) that typically originate from different species. Typically, in these chimeric antibodies, the variable region of both light and heavy chains mimics the variable regions of antibodies derived from one species of mammals, while the constant portions are homologous to the sequences in antibodies derived from another. Most typically, chimeric antibodies comprise human and murine antibody fragments, generally human constant and mouse variable regions. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)). One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy. Methods for humanizing non-human antibodies are well known in the art. Generally, a humanized antibody has one or more amino acid residues introduced into it from a source that is non-human. These non-human amino acid residues are often referred to as import residues, which are typically taken from an import variable domain. Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); Jones et al., Nature 321:522-525 (1986); Morrison et al., Proc. Natl. Acad. Sci, U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu. One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region that disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which are incorporated herein by reference.

Humanized antibodies to CA polypeptides can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. The monoclonal antibodies can be tested for the ability to inhibit or neutralize the biological activity or physiological effect of the corresponding protein.

In the present invention, CA polypeptides of the invention and variants thereof are used to immunize a transgenic animal as described above. Monoclonal antibodies are made using methods known in the art, and the specificity of the antibodies is tested using isolated CA polypeptides. Methods for preparation of the human or primate CA or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E. I. du Pont de Nemours Company, Wilmington, Del.) (Caprino and Han, J. Org Chem. 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified CA proteins usually by ELISA or by bioassay based upon the ability to block the action of CA proteins. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of a CA polypeptide by treatment of a patient with specific antibodies to the CA protein.

Specific antibodies, either polyclonal or monoclonal, to the CA proteins can be produced by any suitable method known in the art as discussed above. For example, murine or human monoclonal antibodies can be produced by hybridoma technology or, alternatively, the CA proteins, or an immunologically active fragment thereof, or an anti-idiotypic antibody, or fragment thereof can be administered to an animal to elicit the production of antibodies capable of recognizing and binding to the CA proteins. Such antibodies can be from any class of antibodies including, but not limited to IgG, IgA, IgM, IgD, and IgE or in the case of avian species, IgY and from any subclass of antibodies.

By immunotherapy is meant treatment of a cancer with an antibody raised against a CA protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In a preferred embodiment, oncogenes which encode secreted growth factors may be inhibited by raising antibodies against CA proteins that are secreted proteins as described above. Without being bound by theory, antibodies used for treatment, bind and prevent the secreted protein from binding to its receptor, thereby inactivating the secreted CA protein.

In another preferred embodiment, the CA protein to which antibodies are raised is a transmembrane protein. Without being bound by theory, antibodies used for treatment, bind the extracellular domain of the CA protein and prevent it from binding to other proteins, such as circulating ligands or cell-associated molecules. The antibody may cause down-regulation of the transmembrane CA protein. As will be appreciated by one of ordinary skill in the art, the antibody may be a competitive, non-competitive or uncompetitive inhibitor of protein binding to the extracellular domain of the CA protein. The antibody is also an antagonist of the CA protein. Further, the antibody prevents activation of the transmembrane CA protein. In one aspect, when the antibody prevents the binding of other molecules to the CA protein, the antibody prevents growth of the cell. The antibody may also sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, cancers may be treated by administering to a patient antibodies directed against the transmembrane CA protein.

In another preferred embodiment, the antibody is conjugated to a therapeutic moiety. In one aspect the therapeutic moiety is a small molecule that modulates the activity of the CA protein. In another aspect the therapeutic moiety modulates the activity of molecules associated with or in close proximity to the CA protein. The therapeutic moiety may inhibit enzymatic activity such as protease or protein kinase activity associated with cancer.

In a preferred embodiment, the therapeutic moiety may also be a cytotoxic agent. In this method, radioisotopes, natural toxins, chemotherapy agents, or other substances (such as biological response modifiers) are chemically linked or conjugated to a monoclonal antibody to form “immunoconjugates” and “immunotoxins” which target the cytotoxic agent to tumor tissue or cells resulting in a reduction in the number of afflicted cells, thereby reducing symptoms associated with cancers, including lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against CA proteins, or binding of a radionuclide to a chelating agent that has been covalently attached to the antibody. Targeting the therapeutic moiety to transmembrane CA proteins not only serves to increase the local concentration of therapeutic moiety in the cancer of interest, i.e., lymphoma, but also serves to reduce deleterious side effects that may be associated with the therapeutic moiety. A number of investigators have used monoclonal antibodies as carriers of cytotoxic substances in attempts to selectively direct those agents to malignant tissue. More particularly, a number of monoclonal antibodies have been conjugated to toxins such as ricin, abrin, diphtheria toxin and Pseudomonas exotoxin or to enzymatically active portions (A chains) thereof via heterobifunctional agents. See, e.g., U.S. Pat. No. 4,753,894 to Frankel et al.; Nevelle, et al. (1982) Immunol Rev 62:75-91; Ross et al. (1980) Eur. J. Biochem 104; Vitteta et al. (1982) Immunol Rev 62:158-183; Raso et al. (1982) Cancer Res 42:457-464, and Trowbridge et al. (1981) Nature 294:171-173.

In another preferred embodiment, the CA protein against which the antibodies are raised is an intracellular protein. In this case, the antibody may be conjugated to a protein that facilitates entry into the cell. In one case, the antibody enters the cell by endocytosis. In another embodiment, a nucleic acid encoding the antibody is administered to the individual or cell. Moreover, wherein the CA protein can be targeted within a cell, e.g., the nucleus, an antibody thereto contains a signal for that target localization, e.g., a nuclear localization signal.

The CA antibodies of the invention specifically bind to CA proteins. By “specifically bind” herein is meant that the antibodies bind to the protein with a binding constant in the range of 10⁻⁴-10⁻⁶ M⁻¹, with a preferred range being 10⁻⁷-10⁻⁹ M⁻¹.

In a preferred embodiment, the CA protein is purified or isolated after expression. CA proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the CA protein may be purified using a standard anti-CA antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the CA protein. In some instances no purification will be necessary.

Detection of Cancer Phenotype

Once expressed and purified if necessary, the CA proteins and nucleic acids are useful in a number of applications. In one aspect, the expression levels of genes are determined for different cellular states in the cancer phenotype; that is, the expression levels of genes in normal tissue and in cancer tissue (and in some cases, for varying severities of lymphoma that relate to prognosis, as outlined below) are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or cancer tissue.

“Differential expression,” or equivalents used herein, refers to both qualitative as well as quantitative differences in the temporal and/or cellular expression patterns of genes, within and among the cells. Thus, a differentially expressed gene can qualitatively have its expression altered, including an activation or inactivation, in, for example, normal versus cancer tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either up-regulated, resulting in an increased amount of transcript, or down-regulated, resulting in a decreased amount of transcript. The degree to which expression differs need only be large enough to quantify via standard characterization techniques as outlined below, such as by use of Affymetrix GeneChip® expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the CA protein and standard immunoassays (ELISAs, etc.) or other techniques, including mass spectroscopy assays, 2D gel electrophoresis assays, etc. Thus, the proteins corresponding to CA genes, i.e. those identified as being important in a particular cancer phenotype, i.e., lymphoma, can be evaluated in a diagnostic test specific for that cancer.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the CA nucleic acid probes may be attached to biochips as outlined herein for the detection and quantification of CA sequences in a particular cell. The assays are done as is known in the art. As will be appreciated by those in the art, any number of different CA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect CA sequences that are up-regulated or down-regulated in cancers as compared to normal tissue. In instances where the CA sequence has been altered but shows the same expression profile or an altered expression profile, the protein will be detected as outlined herein.

In a preferred embodiment nucleic acids encoding the CA protein are detected. Although DNA or RNA encoding the CA protein may be detected, of particular interest are methods wherein the mRNA encoding a CA protein is detected. The presence of mRNA in a sample is an indication that the CA gene has been transcribed to form the mRNA, and suggests that the protein is expressed. Probes to detect the mRNA can be any nucleotide/deoxynucleotide probe that is complementary to and base pairs with the mRNA-and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method pemeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a CA protein is detected by binding the digoxygenin with an anti-digoxygenin secondary antibody and developed with nitro blue tetrazolium and 5-bromo-4-chloro-3-indoyl phosphate.

In a preferred embodiment, any of the three classes of proteins as described herein (secreted, transmembrane or intracellular proteins) are used in diagnostic assays. The CA proteins, antibodies, nucleic acids, modified proteins and cells containing CA sequences are used in diagnostic assays. This can be done on an individual gene or corresponding polypeptide level, or as sets of assays.

As described and defined herein, CA proteins find use as markers of cancers, including lymphomas such as, but not limited to, Hodgkin's and non-Hodgkin's lymphoma. Detection of these proteins in putative cancer tissue or patients allows for a determination or diagnosis of the type of cancer. Numerous methods known to those of ordinary skill in the art find use in detecting cancers. In one embodiment, antibodies are used to detect CA proteins. A preferred method separates proteins from a sample or patient by electrophoresis on a gel (typically a denaturing and reducing protein gel, but may be any other type of gel including isoelectric focusing gels and the like). Following separation of proteins, the CA protein is detected by immunoblotting with antibodies raised against the CA protein. Methods of immunoblotting are well known to those of ordinary skill in the art.

In another preferred method, antibodies to the CA protein find use in in situ imaging techniques. In this method cells are contacted with from one to many antibodies to the CA protein(s). Following washing to remove non-specific antibody binding, the presence of the antibody or antibodies is detected. In one embodiment the antibody is detected by incubating with a secondary antibody that contains a detectable label. In another method the primary antibody to the CA protein(s) contains a detectable label. In another preferred embodiment each one of multiple primary antibodies contains a distinct and detectable label. This method finds particular use in simultaneous screening for a plurality of CA proteins. As will be appreciated by one of ordinary skill in the art, numerous other histological imaging techniques are useful in the invention.

In a preferred embodiment the label is detected in a fluorometer that has the ability to detect and distinguish emissions of different wavelengths. In addition, a fluorescence activated cell sorter (FACS) can be used in the method.

In another preferred embodiment, antibodies find use in diagnosing cancers from blood samples. As previously described, certain CA proteins are secreted/circulating molecules. Blood samples, therefore, are useful as samples to be probed or tested for the presence of secreted CA proteins. Antibodies can be used to detect the CA proteins by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled CA nucleic acid probes to tissue arrays is done. For example, arrays of tissue samples, including CA tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes that indicate diagnosis may differ from those that indicate prognosis.

In a preferred embodiment, the CA proteins, antibodies, nucleic acids, modified proteins and cells containing CA sequences are used in prognosis assays. As above, gene expression profiles can be generated that correlate to cancer, especially lymphoma, severity, in terms of long term prognosis. Again, this may be done on either a protein or gene level, with the use of genes being preferred. As above, the CA probes are attached to biochips for the detection and quantification of CA sequences in a tissue or patient. The assays proceed as outlined for diagnosis.

Screening for CA-Targeted Drugs

In one embodiment, any of the CA sequences as described herein are used in drug screening assays. The CA proteins, antibodies, nucleic acids, modified proteins and cells containing CA sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In another embodiment, the CA proteins, antibodies, nucleic acids, modified proteins and cells containing the native or modified CA proteins are used in screening assays. That is, the present invention provides novel methods for screening for compositions that modulate the cancer phenotype. As above, this can be done by screening for modulators of gene expression or for modulators of protein activity. Similarly, this may be done on an individual gene or protein level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the CA genes herein, a variety of assays to evaluate the effects of agents on gene expression may be executed. In a preferred embodiment, assays may be run on an individual gene or protein level. That is, having identified a particular gene as aberrantly regulated in cancer, candidate bioactive agents may be screened to modulate the gene's regulation. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue gives a 10 fold increase in expression for a candidate agent is desired, etc. Alternatively, where the CA sequence has been altered but shows the same expression profile or an altered expression profile, the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the CA protein and standard inmuunoassays. Alternatively, binding and bioactivity assays with the protein may be done as outlined below.

In a preferred embodiment, gene expression monitoring is done and a number of genes, i.e. an expression profile, is monitored simultaneously, although multiple protein expression monitoring can be done as well.

In this embodiment, the CA nucleic acid probes are attached to biochips as outlined herein for the detection and quantification of CA sequences in a particular cell. The assays are further described below.

Generally, in a preferred embodiment, a candidate bioactive agent is added to the cells prior to analysis. Moreover, screens are provided to identify a candidate bioactive agent that modulates a particular type of cancer, modulates CA proteins, binds to a CA protein, or interferes between the binding of a CA protein and an antibody.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the cancer phenotype, binding to and/or modulating the bioactivity of a CA protein, or the expression of a CA sequence, including both nucleic acid sequences and protein sequences. In a particularly preferred embodiment, the candidate agent suppresses a CA phenotype, for example to a normal tissue fingerprint. Similarly, the candidate agent preferably suppresses a severe CA phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of a CA protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 Daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, or amidification to produce structural analogs.

In one embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening in the methods of the invention. Particularly preferred in this embodiment are libraries of bacterial, fungal, viral, and mammalian proteins, with the latter being preferred, and human proteins being especially preferred.

In another preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In one embodiment, the candidate bioactive agents are nucleic acids. As described generally for proteins, nucleic acid candidate bioactive agents may be naturally occurring nucleic acids, random nucleic acids, or “biased” random nucleic acids. In another embodiment, the candidate bioactive agents are organic chemical moieties, a wide variety of which are available in the literature.

In assays for testing alteration of the expression profile of one or more CA genes, after the candidate agent has been added and the cells allowed to incubate for some period of time, a nucleic acid sample containing the target sequences to be analyzed is prepared. The target sequence is prepared using known techniques (e.g., converted from RNA to labeled cDNA, as described above) and added to a suitable microarray. For example, an in vitro reverse transcription with labels covalently attached to the nucleosides is performed. Generally, the nucleic acids are labeled with a label as defined herein, especially with biotin-FITC or PE, Cy3 and Cy5.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions that allow formation of the label probe hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc. These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data are analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to test for alteration of the expression of the CA genes individually. That is, screening for modulation of regulation of expression of a single gene can be done. Thus, for example, in the case of target genes whose presence or absence is unique between two states, screening is done for modulators of the target gene expression.

In addition, screens can be done for novel genes that are induced in response to a candidate agent. After identifying a candidate agent based upon its ability to suppress a CA expression pattern leading to a normal expression pattern, or modulate a single CA gene expression profile so as to mimic the expression of the gene from normal tissue, a screen as described above can be performed to identify genes that are specifically modulated in response to the agent. Comparing expression profiles between normal tissue and agent treated CA tissue reveals genes that are not expressed in normal tissue or CA tissue, but are expressed in agent treated tissue. These agent specific sequences can be identified and used by any of the methods described herein for CA genes or proteins. In particular these sequences and the proteins they encode find use in marking or identifying agent-treated cells. In addition, antibodies can be raised against the agent-induced proteins and used to target novel therapeutics to the treated CA tissue sample.

Thus, in one embodiment, a candidate agent is administered to a population of CA cells, that thus has an associated CA expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, CA tissue may be screened for agents that reduce or suppress the CA phenotype. A change in at least one gene of the expression profile indicates that the agent has an effect on CA activity. By defining such a signature for the CA phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “CA proteins” or “CAP”. The CAP may be a fragment, or alternatively, be the full-length protein to the fragment encoded by the nucleic acids of Tables 1-7 (human genomic sequences of SEQ ID NOS: 6, 16, 26, 40, 52, 60 and 66, and sequences of SEQ ID NOS: 7, 9, 17, 19, 21, 27, 29, 41, 43, 45, 47, 53, 61, 67, 69, 71 and 73 corresponding to the human mRNAs generated therefrom). In a preferred embodiment, the CAP is selected from the human protein sequences shown in Tables 1-7 (of SEQ ID NOS: 8, 10, 18, 20, 22, 28, 30, 42, 44, 46, 48, 54, 62, 68, 70, 72 and 74). In another embodiment, the sequences are sequence variants as further described herein.

Preferably, the CAP is a fragment approximately 14 to 24 amino acids in length. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, e.g., to a cysteine.

In one embodiment the CA proteins are conjugated to an immunogenic agent as discussed herein. In one embodiment the CA protein is conjugated to BSA.

In a preferred embodiment, screening is done to alter the biological function of the expression product of the CA gene. Again, having identified the importance of a gene in a particular state, screening for agents that bind and/or modulate the biological activity of the gene product can be run as is more fully outlined below.

In a preferred embodiment, screens are designed to first find candidate agents that can bind to CA proteins, and then these agents may be used in assays that evaluate the ability of the candidate agent to modulate the CAP activity and the cancer phenotype. Thus, as will be appreciated by those in the art, there are a number of different assays that may be run; binding assays and activity assays.

In a preferred embodiment, binding assays are done. In general, purified or isolated gene product is used; that is, the gene products of one or more CA nucleic acids are made. In general, this is done as is known in the art. For example, antibodies are generated to the protein gene products, and standard immunoassays are run to determine the amount of protein present. Alternatively, cells comprising the CA proteins can be used in the assays.

Thus, in a preferred embodiment, the methods comprise combining a CA protein and a candidate bioactive agent, and determining the binding of the candidate agent to the CA protein. Preferred embodiments utilize the human or mouse CA protein, although other mammalian proteins may also be used, for example for the development of animal models of human disease. In some embodiments, as outlined herein, variant or derivative CA proteins may be used.

Generally, in a preferred embodiment of the methods herein, the CA protein or the candidate agent is non-diffusably bound to an insoluble support having isolated sample receiving areas (e.g. a microtiter plate, an array, etc.). The insoluble support may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon®g, etc. Microtiter plates and arrays are especially convenient because a large number of assays can be carried out simultaneously, using small amounts of reagents and samples.

The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the CA protein is bound to the support, and a candidate bioactive agent is added to the assay. Alternatively, the candidate agent is bound to the support and the CA protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the CA protein may be done in a number of ways. In a preferred embodiment, the candidate bioactive agent is labeled, and binding determined directly. For example, this may be done by attaching all or a portion of the CA protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using 125, for the proteins, for example, and a fluorophore for the candidate agents.

In a preferred embodiment, the binding of the candidate bioactive agent is determined through the use of competitive binding assays. In this embodiment, the competitor is a binding moiety known to bind to the target molecule (i.e. CA protein), such as an antibody, peptide, binding partner, ligand, etc. Under certain circumstances, there may be competitive binding as between the bioactive agent and the binding moiety, with the binding moiety displacing the bioactive agent.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the CA protein and thus is capable of binding to, and potentially modulating, the activity of the CA protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the CA protein with a higher affinity. Thus, if the candidate bioactive agent is labeled, the presence of the label on the support, coupled with a lack of competitor binding, may indicate that the candidate agent is capable of binding to the CA protein.

In a preferred embodiment, the methods comprise differential screening to identity bioactive agents that are capable of modulating the activity of the CA proteins. In this embodiment, the methods comprise combining a CA protein and a competitor in a first sample. A second sample comprises a candidate bioactive agent, a CA protein and a competitor. The binding of the competitor is determined for both samples, and a change, or difference in binding between the two samples indicates the presence of an agent capable of binding to the CA protein and potentially modulating its activity. That is, if the binding of the competitor is different in the second sample relative to the first sample, the agent is capable of binding to the CA protein.

Alternatively, a preferred embodiment utilizes differential screening to identify drug candidates that bind to the native CA protein, but cannot bind to modified CA proteins. The structure of the CA protein may be modeled, and used in rational drug design to synthesize agents that interact with that site. Drug candidates that affect CA bioactivity are also identified by screening drugs for the ability to either enhance or reduce the activity of the protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of CA proteins may also be done. In a preferred embodiment, methods for screening for a bioactive agent capable of modulating the activity of CA proteins comprise the steps of adding a candidate bioactive agent to a sample of CA proteins, as above, and determining an alteration in the biological activity of CA proteins. “Modulating the activity of a CA protein” includes an increase in activity, a decrease in activity, or a change in the type or kind of activity present. Thus, in this embodiment, the candidate agent should both bind to CA proteins (although this may not be necessary), and alter its biological or biochemical activity as defined herein. The methods include both in vitro screening methods, as are generally outlined above, and in vivo screening of cells for alterations in the presence, distribution, activity or amount of CA proteins.

Thus, in this embodiment, the methods comprise combining a CA sample and a candidate bioactive agent, and evaluating the effect on CA activity. By “CA activity” or grammatical equivalents herein is meant one of the CA protein's biological activities, including, but not limited to, its role in tumorigenesis, including cell division, preferably in lymphatic tissue, cell proliferation, tumor growth and transformation of cells. In one embodiment, CA activity includes activation of or by a protein encoded by a nucleic acid of Tables 1-7. An inhibitor of CA activity is the inhibition of any one or more CA activities.

In a preferred embodiment, the activity of the CA protein is increased; in another preferred embodiment, the activity of the CA protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a CA protein. The methods comprise adding a candidate bioactive agent, as defined above, to a cell comprising CA proteins. Preferred cell types include almost any cell. The cells contain a recombinant nucleic acid that encodes a CA protein. In a preferred embodiment, a library of candidate agents is tested on a plurality of cells.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, bioactive agents are identified. Compounds with pharmacological activity are able to enhance or interfere with the activity of the CA protein.

Applications of the Invention

In one embodiment, a method of inhibiting cancer cell division is provided. In another embodiment, a method of inhibiting tumor growth is provided. In a further embodiment, methods of treating cells or individuals with cancer are provided.

The method comprises administration of a cancer inhibitor. In particular embodiments, the cancer inhibitor is an antisense molecule, a pharmaceutical composition, a therapeutic agent or small molecule, or a monoclonal, polyclonal, chimeric or humanized antibody. In particular embodiments, a therapeutic agent is coupled with a an antibody, preferable a monoclonal antobody.

In other embodiments, methods for detection or diagnosis of cancer cells in an individual are provided. In particular embodiments, the diagnostic/detection agent is a small molecule that pereferentially binds to a CAP according to the invention. In one embodiment, the diagnostic/detection agent is an antibody, preferably a monoclonal antobody, preferably linked to a detectable agent.

In other embodiments of the invention, animal models and transgenic animals are provided, which find use in generating animal models of cancers, particularly lymphomas and carcinomas.

(a) Antisense Molecules

In one embodiment, the cancer inhibitor is an antisense molecule. Antisense molecules as used herein include antisense or sense oligonucleotides comprising a single-stranded nucleic acid sequence (either RNA or DNA) capable of binding to target mRNA (sense) or DNA (antisense) sequences for cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules can be modified or unmodified RNA, DNA, or mixed polymer oligonucleotides. These molecules function by specifically binding to matching sequences resulting in inhibition of peptide synthesis (Wu-Pong, November 1994, BioPharm, 20-33) either by steric blocking or by activating an RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190). In addition, binding of single stranded DNA to RNA can result in nuclease-mediated degradation of the heteroduplex (Wu-Pong, supra). Backbone modified DNA chemistry which have thus far been shown to act as substrates for RNase H are phosphorothioates, phosphorodithioates, borontrifluoridates, and 2′-arabino and 2′-fluoro arabino-containing oligonucleotides.

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

(b) RNA Interference

RNA interference refers to the process of sequence-specific post transcriptional gene silencing in animals mediated by short interfering RNAs (siRNA) (Fire et al., Nature, 391, 806 (1998)). The corresponding process in plants is referred to as post transcriptional gene silencing or RNA silencing and is also referred to as quelling in fungi. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L. (reviewed in Sharp, P. A., RNA interference —2001, Genes & Development 15:485-490 (2001)).

Small interfering RNAs (siRNAs) are powerful sequence-specific reagents designed to suppress the expression of genes in cultured mammalian cells through a process known as RNA interference (RNAi). Elbashir, S. M. et al. Nature 411:494-498 (2001); Caplen, N. J. et al. Proc. Natl. Acad. Sci. USA 98:9742-9747 (2001); Harborth, J. et al. J. Cell Sci. 114:4557-4565 (2001). The term “short interfering RNA” or “siRNA” refers to a double stranded nucleic acid molecule capable of RNA interference “RNAi”, (see Kreutzer et al., WO 00/44895; Zernicka-Goetz et al. WO 01/36646; Fire, WO 99/32619; Mello and Fire, WO 01/29058). As used herein, siRNA molecules are limited to RNA molecules but further encompasses chemically modified nucleotides and non-nucleotides. siRNA gene-targeting experiments have been carried out by transient siRNA transfer into cells (achieved by such classic methods as liposome-mediated transfection, electroporation, or microinjection).

Molecules of siRNA are 21- to 23-nucleotide RNAs, with characteristic 2- to 3-nucleotide 3′-overhanging ends resembling the RNase III processing products of long double-stranded RNAs (dsRNAs) that normally initiate RNAi. When introduced into a cell, they assemble with yet-to-be-identified proteins of an endonuclease complex (RNA-induced silencing complex), which then guides target mRNA cleavage. As a consequence of degradation of the targeted mRNA, cells with a specific phenotype characteristic of suppression of the corresponding protein product are obtained. The small size of siRNAs, compared with traditional antisense molecules, prevents activation of the dsRNA-inducible interferon system present in mammalian cells. This avoids the nonspecific phenotypes normally produced by dsRNA larger than 30 base pairs in somatic cells.

Intracellular transcription of small RNA molecules is achieved by cloning the siRNA templates into RNA polymerase III (Pol III) transcription units, which normally encode the small nuclear RNA (snRNA) U6 or the human RNase P RNA H1. Two approaches have been developed for expressing siRNAs: in the first, sense and antisense strands constituting the siRNA duplex are transcribed by individual promoters (Lee, N. S. et al. Nat. Biotechnol. 20, 500-505 (2002). Miyagishi, M. & Taira, K. Nat. Biotechnol. 20, 497-500 (2002).); in the second, siRNAs are expressed as fold-back stem-loop structures that give rise to siRNAs after intracellular processing (Paul, C. P. et al. Nat. Biotechnol. 20:505-508 (2002)). The endogenous expression of siRNAs from introduced DNA templates is thought to overcome some limitations of exogenous siRNA delivery, in particular the transient loss of phenotype. U6 and H1 RNA promoters are members of the type III class of Pol III promoters. (Paule, M. R. & White, R. J. Nucleic Acids Res. 28, 1283-1298 (2000)).

Co-expression of sense and antisense siRNAs mediate silencing of target genes, whereas expression of sense or antisense siRNA alone do not greatly affect target gene expression. Transfection of plasmid DNA, rather than synthetic siRNAs, may appear advantageous, considering the danger of RNase contamination and the costs of chemically synthesized siRNAs or siRNA transcription kits. Stable expression of siRNAs allows new gene therapy applications, such as treatment of persistent viral infections. Considering the high specificity of siRNAs, the approach also allows the targeting of disease-derived transcripts with point mutations, such as RAS or TP53 oncogene transcripts, without alteration of the remaining wild-type allele. Finally, by high-throughput sequence analysis of the various genomes, the DNA-based methodology may also be a cost-effective alternative for automated genome-wide loss-of-function phenotypic analysis, especially when combined with miniaturized array-based phenotypic screens. (Ziauddin, J. & Sabatini, D. M. Nature 411:107-110 (2001)).

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409:363 (2001)). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., Science, 293, 834 (2001)). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., Genes Dev., 15, 188 (2001)).

This invention provides an expression system comprising an isolated nucleic acid molecule comprising a sequence capable of specifically hybridizing to the CA sequences. In an embodiment, the nucleic acid molecule is capable of inhibiting the expression of the CA protein. A method of inhibiting expression of CA inside a cell by a vector-directed expression of a short RNA which short RNA can fold in itself and create a double strand RNA having CA mRNA sequence identity and able to trigger posttranscriptional gene silencing, or RNA interference (RNAi), of the CA gene inside the cell. In another method a short double strand RNA having CA mRNA sequence identity is delivered inside the cell to trigger posttranscriptional gene silencing, or RNAi, of the CA gene. In various embodiments, the nucleic acid molecule is at least a 7 mer, at least a 10 mer, or at least a 20 mer. In a further embodiment, the sequence is unique.

(c) Pharmaceutical Compositions

Pharmaceutical compositions encompassed by the present invention include as active agent, the polypeptides, polynucleotides, antisense oligonucleotides, or antibodies of the invention disclosed herein in a therapeutically effective amount. An “effective amount” is an amount sufficient to effect beneficial or desired results, including clinical results. An effective amount can be administered in one or more administrations. For purposes of this invention, an effective amount of an adenoviral vector is an amount that is sufficient to palliate, ameliorate, stabilize, reverse, slow or delay the progression of the disease state.

The compositions can be used to treat cancer as well as metastases of primary cancer. In addition, the pharmaceutical compositions can be used in conjunction with conventional methods of cancer treatment, e.g., to sensitize tumors to radiation or conventional chemotherapy. The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

Where the pharmaceutical composition comprises an antibody that specifically binds to a gene product encoded by a differentially expressed polynucleotide, the antibody can be coupled to a drug for delivery to a treatment site or coupled to a detectable label to facilitate imaging of a site comprising cancer cells, such as prostate cancer cells. Methods for coupling antibodies to drugs and detectable labels are well known in the art, as are methods for imaging using detectable labels.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg of the compositions of the present invention in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The pharmaceutical compositions of the present invention comprise a CA protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. Once formulated, the compositions contemplated by the invention can be (1) administered directly to the subject (e.g., as polynucleotide, polypeptides, small molecule agonists or antagonists, and the like); or (2) delivered ex vivo, to cells derived from the subject (e.g., as in ex vivo gene therapy). Direct delivery of the compositions will generally be accomplished by parenteral injection, e.g., subcutaneously, intraperitoneally, intravenously or intramuscularly, intratumoral or to the interstitial space of a tissue. Other modes of administration include oral and pulmonary administration, suppositories, and transdermal applications, needles, and gene guns or hyposprays. Dosage treatment can be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells. Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Once differential expression of a gene corresponding to a CA polynucleotide described herein has been found to correlate with a proliferative disorder, such as neoplasia, dysplasia, and hyperplasia, the disorder can be amenable to treatment by administration of a therapeutic agent based on the provided polynucleotide, corresponding polypeptide or other corresponding molecule (e.g., antisense, ribozyme, etc.). In other embodiments, the disorder can be amenable to treatment by administration of a small molecule drug that, for example, serves as an inhibitor (antagonist) of the function of the encoded gene product of a gene having increased expression in cancerous cells relative to normal cells or as an agonist for gene products that are decreased in expression in cancerous cells (e.g., to promote the activity of gene products that act as tumor suppressors).

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic compositions agents includes local or systemic administration, including injection, oral administration, particle gun or catheterized administration, and topical administration. Preferably, the therapeutic polynucleotide composition contains an expression construct comprising a promoter operably linked to a polynucleotide of at least 12, 22, 25, 30, or 35 contiguous nt of the polynucleotide disclosed herein. Various methods can be used to administer the therapeutic composition directly to a specific site in the body. For example, a small metastatic lesion is located and the therapeutic composition injected several times in several different locations within the body of tumor. Alternatively, arteries that serve a tumor are identified, and the therapeutic composition injected into such an artery, in order to deliver the composition directly into the tumor. A tumor that has a necrotic center is aspirated and the composition injected directly into the now empty center of the tumor. An antisense composition is directly administered to the surface of the tumor, for example, by topical application of the composition. X-ray imaging is used to assist in certain of the above delivery methods.

Targeted delivery of therapeutic compositions containing an antisense polynucleotide, subgenomic polynucleotides, or antibodies to specific tissues can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g., for enhancing or inhibiting levels of the encoded gene product) and efficacy of transformation and expression are considerations that will affect the dosage required for ultimate efficacy of the antisense subgenomic polynucleotides. Where greater expression is desired over a larger area of tissue, larger amounts of antisense subgenomic polynucleotides or the same amounts re-administered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell Biol. (1994) 14:2411, and in Woffendin, Proc. Natl. Acad. Sci. (1994) 91:1581.

Further non-viral delivery suitable for use includes mechanical delivery systems such as the approach described in Woffendin et al., Proc. Natl. Acad. Sci. USA (1994) 91(24):11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

The administration of the CA proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the CA proteins and modulators may be directly applied as a solution or spray.

In a preferred embodiment, CA proteins and modulators are administered as therapeutic agents, and can be formulated as outlined above. Similarly, CA genes (including both the fill-length sequence, partial sequences, or regulatory sequences of the CA coding regions) can be administered in gene therapy applications, as is known in the art. These CA genes can include antisense applications, either as gene therapy (i.e. for incorporation into the genome) or as antisense compositions, as will be appreciated by those in the art.

Thus, in one embodiment, methods of modulating CA gene activity in cells or organisms are provided. In one embodiment, the methods comprise administering to a cell an anti-CA antibody that reduces or eliminates the biological activity of an endogenous CA protein. Alternatively, the methods comprise administering to a cell or organism a recombinant nucleic acid encoding a CA protein. As will be appreciated by those in the art, this may be accomplished in any number of ways. In a preferred embodiment, for example when the CA sequence is down-regulated in cancer, the activity of the CA gene product is increased by increasing the amount of CA expression in the cell, for example by overexpressing the endogenous CA gene or by administering a gene encoding the CA sequence, using known gene-therapy techniques. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the CA sequence is up-regulated in cancer, the activity of the endogenous CA gene is decreased, for example by the administration of a CA antisense nucleic acid.

(d) Vaccines

In a preferred embodiment, CA genes are administered as DNA vaccines, either single genes or combinations of CA genes. Naked DNA vaccines are generally known in the art. Brower, Nature Biotechnology, 16:1304-1305 (1998).

In one embodiment, CA genes of the present invention are used as DNA vaccines. Methods for the use of genes as DNA vaccines are well known to one of ordinary skill in the art, and include placing a CA gene or portion of a CA gene under the control of a promoter for expression in a patient with cancer. The CA gene used for DNA vaccines can encode full-length CA proteins, but more preferably encodes portions of the CA proteins including peptides derived from the CA protein. In a preferred embodiment a patient is immunized with a DNA vaccine comprising a plurality of nucleotide sequences derived from a CA gene. Similarly, it is possible to immunize a patient with a plurality of CA genes or portions thereof. Without being bound by theory, expression of the polypeptide encoded by the DNA vaccine, cytotoxic T-cells, helper T-cells and antibodies are induced that recognize and destroy or eliminate cells expressing CA proteins.

In a preferred embodiment, the DNA vaccines include a gene encoding an adjuvant molecule with the DNA vaccine. Such adjuvant molecules include cytokines that increase the immunogenic response to the CA polypeptide encoded by the DNA vaccine. Additional or alternative adjuvants are known to those of ordinary skill in the art and find use in the invention.

(e) Antibodies

In one embodiment, a cancer inhibitor is an antibody as discussed above. In one embodiment, the CA proteins of the present invention may be used to generate polyclonal and monoclonal antibodies to CA proteins, which are useful as described herein. Similarly, the CA proteins can be coupled, using standard technology, to affinity chromatography columns. These columns may then be used to purify CA antibodies. In a preferred embodiment, the antibodies are generated to epitopes unique to a CA protein; that is, the antibodies show little or no cross-reactivity to other proteins. These antibodies find use in a number of applications. For example, the CA antibodies may be coupled to standard affinity chromatography columns and used to purify CA proteins. The antibodies may also be used therapeutically as blocking polypeptides, as outlined above, since they will specifically bind to the CA protein.

The present invention further provides methods for detecting the presence of and/or measuring a level of a polypeptide in a biological sample, which CA polypeptide is encoded by a CA polynucleotide that is differentially expressed in a cancer cell, using an antibody specific for the encoded polypeptide. The methods generally comprise: a) contacting the sample with an antibody specific for a polypeptide encoded by a CA polynucleotide that is differentially expressed in a prostate cancer cell; and b) detecting binding between the antibody and molecules of the sample.

Detection of specific binding of the antibody specific for the encoded cancer-associated polypeptide, when compared to a suitable control is an indication that encoded polypeptide is present in the sample. Suitable controls include a sample known not to contain the encoded CA polypeptide or known not to contain elevated levels of the polypeptide; such as normal tissue, and a sample contacted with an antibody not specific for the encoded polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

In some embodiments, the methods are adapted for use in vivo, e.g., to locate or identify sites where cancer cells are present. In these embodiments, a detectably-labeled moiety, e.g., an antibody, which is specific for a cancer-associated polypeptide is administered to an individual (e.g., by injection), and labeled cells are located using standard imaging techniques, including, but not limited to, magnetic resonance imaging, computed tomography scanning, and the like. In this manner, cancer cells are differentially labeled.

(f) Detection and Diagnosis of Cancers

Without being bound by theory, it appears that the various CA sequences are important in cancers. Accordingly, disorders based on mutant or variant CA genes may be determined. In one embodiment, the invention provides methods for identifying cells containing variant CA genes comprising determining all or part of the sequence of at least one endogenous CA genes in a cell. As will be appreciated by those in the art, this may be done using any number of sequencing techniques. In a preferred embodiment, the invention provides methods of identifying the CA genotype of an individual comprising determining all or part of the sequence of at least one CA gene of the individual. This is generally done in at least one tissue of the individual, and may include the evaluation of a number of tissues or different samples of the same tissue. The method may include comparing the sequence of the sequenced CA gene to a known CA gene, i.e., a wild-type gene. As will be appreciated by those in the art, alterations in the sequence of some CA genes can be an indication of either the presence of the disease, or propensity to develop the disease, or prognosis evaluations.

The sequence of all or part of the CA gene can then be compared to the sequence of a known CA gene to determine if any differences exist. This can be done using any number of known homology programs, such as Bestfit, etc. In a preferred embodiment, the presence of a difference in the sequence between the CA gene of the patient and the known CA gene is indicative of a disease state or a propensity for a disease state, as outlined herein.

In a preferred embodiment, the CA genes are used as probes to determine the number of copies of the CA gene in the genome. For example, some cancers exhibit chromosomal deletions or insertions, resulting in an alteration in the copy number of a gene.

In another preferred embodiment CA genes are used as probes to determine the chromosomal location of the CA genes. Information such as chromosomal location finds use in providing a diagnosis or prognosis in particular when chromosomal abnormalities such as translocations, and the like are identified in CA gene loci.

The present invention provides methods of using the polynucleotides described herein for detecting cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of a polynucleotide that is differentially expressed in a cancer cell, and/or detection of a polypeptide encoded by a polynucleotide that is differentially expressed in a cancer cell. The detection methods of the invention can be conducted in vitro or in vivo, on isolated cells, or in whole tissues or a bodily fluid e.g., blood, plasma, serum, urine, and the like).

In some embodiments, methods are provided for detecting a cancer cell by detecting expression in the cell of a transcript that is differentially expressed in a cancer cell. Any of a variety of known methods can be used for detection, including, but not limited to, detection of a transcript by hybridization with a polynucleotide that hybridizes to a polynucleotide that is differentially expressed in a prostate cancer cell; detection of a transcript by a polymerase chain reaction using specific oligonucleotide primers; in situ hybridization of a cell using as a probe a polynucleotide that hybridizes to a gene that is differentially expressed in a prostate cancer cell. The methods can be used to detect and/or measure mRNA levels of a gene that is differentially expressed in a cancer cell. In some embodiments, the methods comprise: a) contacting a sample with a polynucleotide that corresponds to a differentially expressed gene described herein under conditions that allow hybridization; and b) detecting hybridization, if any.

Detection of differential hybridization, when compared to a suitable control, is an indication of the presence in the sample of a polynucleotide that is differentially expressed in a cancer cell. Appropriate controls include, for example, a sample that is known not to contain a polynucleotide that is differentially expressed in a cancer cell, and use of a labeled polynucleotide of the same “sense” as the polynucleotide that is differentially expressed in the cancer cell. Conditions that allow hybridization are known in the art, and have been described in more detail above. Detection can also be accomplished by any known method, including, but not limited to, in situ hybridization, PCR (polymerase chain reaction), RT-PCR (reverse transcription-PCR), TMA, bDNA, and Nasbau and “Northern” or RNA blotting, or combinations of such techniques, using a suitably labeled polynucleotide. A variety of labels and labeling methods for polynucleotides are known in the art and can be used in the assay methods of the invention. Specificity of hybridization can be determined by comparison to appropriate controls.

Polynucleotides generally comprising at least 10 nt, at least 12 nt or at least 15 contiguous nucleotides of a polynucleotide provided herein, such as, for example, those having the sequence as depicted in Tables 1-7, are used for a variety of purposes, such as probes for detection of and/or measurement of, transcription levels of a polynucleotide that is differentially expressed in a prostate cancer cell. As will be readily appreciated by the ordinarily skilled artisan, the probe can be detectably labeled and contacted with, for example, an array comprising immobilized polynucleotides obtained from a test sample (e.g., mRNA). Alternatively, the probe can be immobilized on an array and the test sample detectably labeled. These and other variations of the methods of the invention are well within the skill in the art and are within the scope of the invention.

Nucleotide probes are used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluorophores, and enzymes. Other examples of nucleotide hybridization assays are described in WO92/02526 and U.S. Pat. No. 5,124,246.

PCR is another means for detecting small amounts of target nucleic acids (see, e.g., Mullis et al., Meth. Enzymol. (1987) 155:335; U.S. Pat. No. 4,683,195; and U.S. Pat. No. 4,683,202). Two primer oligonucleotides that hybridize with the target nucleic acids are used to prime the reaction. The primers can be composed of sequence within or 3′ and 5′ to the CA polynucleotides disclosed herein. Alternatively, if the primers are 3′ and 5′ to these polynucleotides, they need not hybridize to them or the complements. After amplification of the target with a thermostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Methods using PCR amplification can be performed on the DNA from a single cell, although it is convenient to use at least about 10⁵ cells. The use of the polymerase chain reaction is described in Saiki et al. (1985) Science 239:487, and a review of current techniques may be found in Sambrook, et al. Molecular Cloning: A Laboratory Manual, CSH Press 1989, pp. 14.2-14.33. A detectable label may be included in the amplification reaction. Suitable detectable labels include fluorochromes, (e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²p, ³⁵S, ³H, etc.), and the like. The label may be a two stage system, where the polynucleotides is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The detection methods can be provided as part of a kit. Thus, the invention further provides kits for detecting the presence and/or a level of a polynucleotide that is differentially expressed in a cancer cell (e.g., by detection of an mRNA encoded by the differentially expressed gene of interest), and/or a polypeptide encoded thereby, in a biological sample. Procedures using these kits can be performed by clinical laboratories, experimental laboratories, medical practitioners, or private individuals. The kits of the invention for detecting a polypeptide encoded by a polynucleotide that is differentially expressed in a cancer cell may comprise a moiety that specifically binds the polypeptide, which may be an antibody that binds the polypeptide or fragment thereof. The kits of the invention used for detecting a polynucleotide that is differentially expressed in a prostate cancer cell may comprise a moiety that specifically hybridizes to such a polynucleotide. The kit may optionally provide additional components that are useful in the procedure, including, but not limited to, buffers, developing reagents, labels, reacting surfaces, means for detection, control samples, standards, instructions, and interpretive information. Accordingly, the present invention provides kits for detecting prostate cancer comprising at least one of polynucleotides having the sequence as shown in Tables 1-7 or fragments thereof.

The present invention further relates to methods of detecting/diagnosing a neoplastic or preneoplastic condition in a mammal (for example, a human). “Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

An “effective amount” is an amount sufficient to effect beneficial or desired results, including clinical results. An effective amount can be administered in one or more administrations.

A “cell sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “cell sample” encompasses a clinical sample, and also includes cells in culture, cell supernatants, cell lysates, serum, plasma, biological fluid, and tissue samples.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign.

The terms “individual,” “subject,” “host,” and “patient,” are used interchangeably herein and refer to any mammalian subject for whom diagnosis, treatment, or therapy is desired, particularly humans. Other subjects may include cattle, dogs, cats, guinea pigs, rabbits, rats, mice, horses, and so on. Examples of conditions that can be detected/diagnosed in accordance with these methods include cancers. Polynucleotides corresponding to genes that exhibit the appropriate expression pattern can be used to detect cancer in a subject. For a review of markers of cancer, see, e.g., Hanahan et al. Cell 100:57-70 (2000).

One detection/diagnostic method comprises: (a) obtaining from a mammal (e.g., a human) a biological sample, (b) detecting the presence in the sample of a CA protein and (c) comparing the amount of product present with that in a control sample. In accordance with this method, the presence in the sample of elevated levels of a CA gene product indicates that the subject has a neoplastic or preneoplastic condition.

Biological samples suitable for use in this method include biological fluids such as serum, plasma, pleural effusions, urine and cerebro-spinal fluid, CSF, tissue samples (e.g., mammary tumor or prostate tissue slices) can also be used in the method of the invention, including samples derived from biopsies. Cell cultures or cell extracts derived, for example, from tissue biopsies can also be used.

The compound is preferably a binding protein, e.g., an antibody, polyclonal or monoclonal, or antigen binding fragment thereof, which can be labeled with a detectable marker (e.g., fluorophore, chromophore or isotope, etc). Where appropriate, the compound can be attached to a solid support such as a bead, plate, filter, resin, etc. Determination of formation of the complex can be effected by contacting the complex with a further compound (e.g., an antibody) that specifically binds to the first compound (or complex). Like the first compound, the further compound can be attached to a solid support and/or can be labeled with a detectable marker.

The identification of elevated levels of CA protein in accordance with the present invention makes possible the identification of subjects (patients) that are likely to benefit from adjuvant therapy. For example, a biological sample from a post primary therapy subject (e.g., subject having undergone surgery) can be screened for the presence of circulating CA protein, the presence of elevated levels of the protein, determined by studies of normal populations, being indicative of residual tumor tissue. Similarly, tissue from the cut site of a surgically removed tumor can be examined (e.g., by immunofluorescence), the presence of elevated levels of product (relative to the surrounding tissue) being indicative of incomplete removal of the tumor. The ability to identify such subjects makes it possible to tailor therapy to the needs of the particular subject. Subjects undergoing non-surgical therapy, e.g., chemotherapy or radiation therapy, can also be monitored, the presence in samples from such subjects of elevated levels of CA protein being indicative of the need for continued treatment. Staging of the disease (for example, for purposes of optimizing treatment regimens) can also be effected, for example, by biopsy e.g., with antibody specific for a CA protein.

(g) Animal Models and Transgenics

In another preferred embodiment CA genes find use in generating animal models of cancers, particularly lymphomas and carcinomas. As is appreciated by one of ordinary skill in the art, when the CA gene identified is repressed or diminished in CA tissue, gene therapy technology wherein antisense RNA directed to the CA gene will also diminish or repress expression of the gene. An animal generated as such serves as an animal model of CA that finds use in screening bioactive drug candidates. Similarly, gene knockout technology, for example as a result of homologous recombination with an appropriate gene targeting vector, will result in the absence of the CA protein. When desired, tissue-specific expression or knockout of the CA protein may be necessary.

It is also possible that the CA protein is overexpressed in cancer. As such, transgenic animals can be generated that overexpress the CA protein. Depending on the desired expression level, promoters of various strengths can be employed to express the transgene. Also, the number of copies of the integrated transgene can be determined and compared for a determination of the expression level of the transgene. Animals generated by such methods find use as animal models of CA and are additionally useful in screening for bioactive molecules to treat cancer.

Characterization of CA Sequences

The CA nucleic acid sequences of the invention are depicted in Tables 1-7. The sequences in each Table include genomic DNA sequence (mouse genomic sequences mDxx-yyy; human genomic sequences hDxx-yyy), sequence corresponding to the mRNA(s) generated therefrom (mRxx-yyy; hRxx-yyy) and amino acid sequences of the proteins (mPxx-yyy; hPxx-yyy) encoded by the mRNA for both mouse and human genes. N/A indicates a gene that has been identified, but for which there has not been a name ascribed.

TABLE 1

```
MOUSE GENOMIC SEQUENCE:mD22-022 (Seq ID No: 1)
CGCCCTCGCTCGCAAACCCAAACACTCCCGGCTGCTGGTGCATGTGATCTCCCAGTAGTCGCTCGGCAGAGATGTTGCTGTTGGCCG
CTGCCGGCCTCGTGGCCTTCGTGCTGCTCCTCTACATGGTGTCGCCGCTCATCAGTCCCAAGCCCCTCGCGCTGCCCGGCGCGCACG
TAGTGGTGAGTGGCCTCCTGTTGCTGAGCTTGCCCAGGACTGCTTGGCCGGCTTGGGCCAACGAGACCACGGTCCCGGAAAGGAACG
GTTGAGTGGGGCGGGTTACCTACTGCCTTAGCCCCGGCATTGCCCTGCCCTGTCGGACCTCAGGAACCCGCCCTCCAAGGCGCCGGG
CTACAGGGCAGTGTCGTGGAGAAGGCGCCAGCCTAGTTTCCAGGACTCAGGGGCTCTGCTGCTCGCTGATCCCCGGAGCATCCCTGT
CTGCCACTGTTTGGATCTTCAGTTTCCCCAATGAGTCAACAGAGAGACTGAGAAACTTTGCCAAGGACACGGCAGTCGGTGACTAGC
AGGATAAGGGCGCAAACCCGGATGGGAGTAGCCACAGCCAGGGAATCCTAAGGAGCCCACAGCAAGGAGGGGGTCTGGGCCTCTGGG
GCTGGCCCAGGCAGCTGAGACTGTAGGGTGTCGTGTGCAAGTGACAAAGAGCCCCCAGAGGCTTCTAGGAAGTGATACACCTGAGGG
GTGGCCTGGCAGGTTTGGCCAGCAGGCTTGTCTCTGAACTCAGCCAGGGTCTGCTGAGTACTGCTCAAGTGCAGTCCTTTCTGGCGT
CAGAAGGAAGAGCTAACCAGCTTGATTAGAAGCTTAAGATGGAAGGAGATATGAGTGGTTTGGTTTGTTAAAGTCCGGAAGCTCGCA
CGTTCCTATGTGCAGGCTTGGCTATGCCTCCACCCCGCCCCCACAGCTCAGAGCCTCAACTCTGGCATTTCTCCTCGGTGTCCTATA
TAGACCCAAAACCCGAGGCACAGTGAGGCGAGGAGTTGCGAATAAGGACAGCTAGTATCCAAACCGGCAGCACGAATCTCCTTGGTG
CCCTTAGGTGACTTACATGAGTGCATCCAAGTAAGTCCCCACTGCGTTGTTACAGGATAGAGTGGTTATCTTAGTATGATTTATCCC
AAACAAGAATTTCTGGATGACAACATGTGTTTCCATTAATTTGTCTTTTGTAAAATTACTGATTTATTCCCAGCTGCTGCTATGCAT
GATGTGGAAAGAGTTATTAGAAAAAGCCAAAGTTTACAATCATGCCAAGTTGCTAAAGTTTTACATCCACAAAATCGTGACTAAAGC
AGTTGAACTTGCCGGCTGAGGAAATGGCTCAGTTGGTAAAGTGCTTGCCGTGCATCATGCCCTGAGTTAGGGTCCCTAGCACCCACA
TGCTCACTGGGCCTATGCCTCTAACTTCAGCACTGGGGCCACAACCTGTGGATCCCTGGAGCTCACTGGATAGTTTGCCTAGACGCA
CGAATGATCTTCAGGTTCAGGGAGAAGCCTTGTCGCAAAAATTAAGGTGCAGGGTGACTGAAATGCCAGACACTGCCAGCCTCTGCA
CAGGTGCACCTACATACACAACGCTGAATCAACTTGTGCTTGTATAGCTCAGCTGTGGGGCAAGGGAGGAGAGCCAAAGTCAGAATG
AAAACTAATTCGACAACTACAAGGTACTCACAACTACCCTTGGTACTTGTTCTTGTGTCCCCAGTCCCCGGCTCTTTCACAGAGGAG
GTTAGGGCTGTTTCAATAGACGCATTCTTTTTCCCAACATATTTTTATTGATTCTTTGGGAACTTTACATAATGCACTCCAAAACAC
TCACTTCCCAGCCCTCCCAGGATCCTGCTTTAATACAATAAAAAATAATTACCGCTATGCATGTATGTATGTTTACTGGTATAAACA
ACATAGCATGGCTTATTCATATGGTGTCCCTATCTGCCTTTTTTAATATCTGAAGAGTAAAATGTGATTTAGTTGTGATCTGGGATC
AGGAGAGTGAGCCAAGCTCTTTGTTTTTAGAGCAGTTCACATACGCTGGTGCTGTGGTGTCTGATCACAGCAGCAAAGCTTTGCTGG
TCTTCTCATCCAGTAGATGATTGTTCAGTGTCCCTCCTTCTGGATGACTGAGAGTAGTGCTCCTAGCAGCAGGAAAGTTGCTGGTGC
CCTTAGTAGGGCCTGTATGCTGCGCTTAAAGAGACAGTATCGTTTAAATGGTATTTGTAGTGATCAGTAACAAGGAGTGGTCAAGGA
AGGGAAAGTAAATGGTTATCTTCGGGGCCCTCTGTTTAGATATAGTATACATTTAACGTTTTCTCGGCCATTGTGAAAAACCCACGC
CAGTTCTTAGAGCATCCAGAAGAACATATTCCAGAATTAGCCTAACTGTATTCTTATATGCCACTCAAGACTTAGCTGCGTTGGCTA
GATCCCTTTGAATGCACCTTGATCTGAAAACCAGGTCTCACTCCCAATGTGAGAAAGTGACACTGTTTGCTTTCCTGAAACCCGCCC
TGGCTTTTGTTCACATAATACATCAGAGCATAAAAGATGTCTCAGTAACCTCATTCCTAACTCCATCAGCTTTCTGAACAATAAATA
GGGAGACTTTTATTTGTGAGCTTTGACGTTTTGTTTTGTTTTGGTTTTTTGTTTTCATGGAAATGACATAGGAGAACATTCTGCAAA
CTGAGGAAAGTATTTATTGAGTGCCTGCTTGTGCAGGGGCCTGGCATGGTCTGTGATGAGTGAGACAGGCTGGCTTGGTATTGGTCC
ATAGGAGCCATGAGTTCAGCCAGGGAGCCAAGATATATCCAAAGTGAGTGCAGACTACATGGTGACCAGCCAGGAGGACTGACTGAC
TGATGTGTTCAGGAAAGGTTCCCATAGGGCGTGGGGGATGTGTGGGGGGATGTGTGATCCGGGGAAGATAAACAGCTTAAGGCAGCA
TTATAAAGCATGTGAGGAAATGTTTTAGAGGCTCTGACAAGTACTATGAGGAGTAAAAGTTCATAAACCAGAACAGCCCGTAAGATA
GCCTGATATCAGAAGCCACGGAGATAACACTCAGCCAAGGCAACGTGTATCCTTCTATAGTCATCCTCAGCAGACATTTGGTTAATG
ATCTCCCAAGCAAACCTCTCCATTAGATCCAAGGTGAGACTACAGGGAAGTGACATATTCTCAAGAACACAGAGAGACACATCTATG
CTTGGAGGGTTTCATGGCAATACGAACATTACAGTGTGGGAGATGTTCTTTCTGCATGGCTTTAGAGTAGCAAAATATTCAAAGTAA
TCCAAATGTCTGACTGGAAAGGAGCTAGGTTTTGACAGGAGAGATTTTTGTTTTGCTTTTGATACAGGGTCTTACCGTATAGCCATG
CTGGCTTGGAATTTACTGTGTGGGCCAGAATGGCTTGGATTCGGACAGATAGCCTGCCTCTGCCTCCTGAGTTCTAATATTAAGGTG
TATGCCATCACTACTATGCCTGGCCTGGATGGAGCTCTTTAAATACAGTATTGTGAAGCCTTGTAGTTTTCAACGTTGAAAGGATAT
TCTTTCTTTCTCTCTGCTCACGTTTTTACATAGACTTAGACTTACATAGATGTGCTGGAAGGAAATGAGATTCGTTTGTGTTTGTAG
TCAGCAGCACATAATGTATTATTTCTTAGCTAAAATTGTAGGGCACGTTTCCTTTGGTGACCTGAAACGATTTTAGATGAATAGTTT
ATAGCTCAGCACGGGGGGGGGGGGGGGGCTAGGAGGCTAGGAGGCTCTGCTGAGAGTGTAATCTTTGATGCTTACACTGCATTCCAG
GTGACAAATGTGGTATGAGGAGGTTGTGGTTCTTGGAGACGCTTTGTTCCTGAGACAGAGCCCCATGTAGTCCTAGCCAATACCATG
AAACTGTCCGTGAGATTGAACGACTTCAACCTTGACCTCCTCATCCCCTGCAGACACGCTCCTGGGGAATTTTTAAAACTCAGAATT
ATGTCATAGTCCTATGCAGTACTGGAGATCCAACCTGGGGCCTCATGAATCCTAGGCAGGTACCTGCACTGAGCTACACCCTTACTA
CTCCCAACACAAGTATTTCAAGAAGTCTCTGTACAGAGGAGATATGGCAAGTGCTAAGATGGTCTAAGATGGTCTTCCTGATGTTCT
TCATAGGTCACAGGAGGCTCCAGTGGCATTGGGAAGTGCATTGCTATTGAGTGCTACAAACAAGGAGCATTTATAACTCTGGTTGCA
CGAAATGAGGTAAGCGCTCTAGACTCCTCATGACAAACTCACTAACGCGGGAAGTCCAAGAGACGGGTCTTATCTGTCATTTCTCTC
TGTAACTTGATGATGACATGATAGGCATCTTTGGCAGAGTCCCCATGCCCCAGGAGAGTTTCATTCTTTCCAGTATGTGTAGCAAAG
TAGTTTTTCCTGTTCTAAGCAATCAGGGGTCTGTTTTTCATTGTATTGGGGTCTGCCGTCTTGCCAGAATTGCTTTCGGAGCATGAA
GCTATGACTGAAGATGCCCCTTGTTCCTCTTCAGGGTCTGGAGCAGAGACCCCGGCTTCATGGTCAAAAGGTTGGCCTTGCTTGCTG
TGGAGCAGATAGATACTCTGTTGAGTGATGAAATGAAAGAAGAGGGGCTTGGGGTGTGCTCAGTGGGCTCAGCACTTACCCAGGGTG
AATGAATCAAATCCACAAAGCACTCCAAGAAGGTGCATGGGAACCCTAGCCTTAGGGAGGTGGACGCAGAAGGATCAGAAGTTCAAA
GTCTCCCCCTGCTATACAGCAAAGGAGAGTCTTGCCTGACTTTCATGAGACCCTGTAATTCAGGGACAGGGAAGAAAAAGAAATGAG
ATGTGCTTGAGGGGTTAACTGTCGACATAGAGCGGTTGGTTGAAAAGGGGCTAGTGGAGAAGACAGGCTAGAAAGGATCTCTGGAGA
AAATACAAAACAATAATGATCCAGAACTAGGTTAAAATAGCCATCATTTCCTTAAAGAACAAGTAAATGGTTTTTAAAAGAAATTAT
ACAGCCAGGCGTGGTGGTGCACGCCTTTAATCCCAGCACTTGGGAGGCAGAGGCAGGTGGATTTCTGAGTTCGAGGCCAGCCTGGTC
TACAAAGTGAGTTCCAGGACAGCCAGGGCTATACAGAGAAACCCTGTCTCAAAAAACTCAAAAAAAATTATATATATATTATATATA
TACATATATATATATATATATATACACACACATACATACATACACACACACACACACACACATATATATATATTATGGCTTGGTTGG
ACACACTTTAAGGTCACCATACATGAACAGAACAACAGATTTAGGAAAAGGCCACTTTTGACTTTTTAGCAGGGCTAGAATTTGACA
GTGAGTGAGTGGGCGGGGACTGGTGTCAGAGCTGGAAGGAACTTGCAGGACCGTGTTTGGGCTGGCGTCACAGCTAGTGGGTAGGAA
CTCATGCACAGCCTGGTTGGAGTGAGTATCCAGAGAGTTTTCTAGCCTGAAGATGTTCTTTAGTTTGTCATGTAACTTTTCATTGCA
GTCTTGAATTCATTTCCCATCATTCGATAGCATTGTACTATTTTGTCATGAGCATTCATGTTTGTAAATTCGATTTGTTCTTTGAAG
ATACCTACTGGCCCACCCCCACCCCTCTATGAGACAAGGTCTCGAGTATCATGGGCTAACCTGGAACTCTGTAGCAGAGGATATCCT
TGCCAGATACCTAGGTTCCTCCAGGCTTGGTTTTAGCCTTTCCAGGGATTGACCTCACGGTGCCATGCATGCTGGGACCTTCTTTGT
TCTTCTACAGTTCTCAAATTTTATTTAAATTTATGAAAAATTAAGGTTCTGTAAACTCATCTGTTTTTATGAGAATAAGCAAAACAA
AATGGAGGACATGGGAAAACTTTGCAATTATCTGGGTAGGTTTGTAAGTCTGAGATATATTTTTGAAAGTAACCAGATGTAGATTTT
TCTTTCTCCTCTTTGTAAGAAGTGATTGATTTTTACTCGATGTTGCATTGAAAGGAGTAAACTGCTGATATTATCTAATAAAGCCCT
CCTGGTGTGGAGTCAGCTGCATGCCTTTAGCCTTCCAGAGCATTTGTGGTCGGTCATGTGCTTATGCTGTGTCCCCGCTCAGTGGTG
GGAATGAAAATATCTGTTGGCGTGGACAGGCTGACCAGCAACACTAATGCTGCTGAGAGGGATTTGAGCTTGCTTTCTTCTTCTTCC
CATTCTCTGCAATGGACTTAAATGAACCCTGCTGTGACTTACCTGGGCTCTTAGGTGTTTTGCCTCTTTCTTCCTCAAGCCCACAGC
TCTGATTTATAGAATGCAGTGTCTGTTCAAATGTTGCTTTTGCTTTTCTTTTTAACAAGTGCAGACAGACATAATTTTTTTCTGTAT
TGCTTTTAGGACAAGCTACTGCAGGCGAAGAAAGACATTGAAAAGCACTCTATTAATGACAAACAGGTAAAGTGGGGCCGAAGGAGG
AATTTTCTTTCCTGATAGACAGTTGTTTAAAGGGGAGGAAAGATGTTACTTCCACTGCAACTGGTTCACTGAAGGCATTTATGGTTT
TTCCCAATTAAAATACATTTTAGGCCGCTTTCTAAAAGATGGAATTATTTGAAATGACTAGAATTCAGGTCTCTGTACAGAAACCAG
ATGGTATAGACTATCACACATACCTGAAAATAGCCAAGCAGCGCTGTATGAGAAAATGGTTTCGTTGCAATTACTCAATTTACACAA
CGACTTAGATAGAAAGGATGTTTGAAAAGATGTTGTATGATTTGGGGTACTAAACTGATACAGACACTGTAATGTATATTTTCATGT
GCAAGTCAGTGACTGGTTGACTACTACAGATCCACCATCTAATCTAAACAAATGGAAATTAAAGGACTTTCTGAACCCTCTCTCCTG
CCTTTGGGCTCATTACCAGCCTCTCGTTCTTAGAGGCCACCTTAAGCCCGACTCTGATGTTGTTGTTCATTCATTCTTTACTTTTCT
TTAAGATTTTACCGTCTCTGGTAGTGTGTATAGTCTTTTGTATGAGTGTGTGTGTGCACCTGTGTGTACAACGGTCCTGATATGCAT
GCACATACGTGCACATGCTCGAGAAGCCACGGGCCAACATTGGGTATCTTCTGTAATCTCTCCATATTTTTTTTTTTTAAATAGGCT
CTTTTGGTGAGCCTGAAGCTATCCTTGCTGGGGATCTAAACGCAGGGCCTCCTCACGCTAGCACCTTGCCAGCTAGTCATCTTAGCA
CCTAAATAGTGCGTGTGCGTGTGCGTGTGTGCGTGTGTGTTTGTGTGTTTTCTGTATCCTTTTGAGCTTTGGCAGCATTCCTATGTT
TCTGAACTGCCTTAATGAGACCTCGCACACAGTATGTAGTTATGCTTAAGACACCCACTTCCTGAGTTTTCACAAAGTTGTGCAATC
ATAATCACAATCAATTTGGAAATTGTCCCAACCCTGAAGTCTCCAGACTGCCATTAGCAGCCACACCCATTCCTGCCCTGCCTCTCC
TAGCCCTAGCCIAGTACTAATAACCTTTATAATTTGATAGACTGATGGACATTCTTATGCAGTGTCCTGCAACCTTATTTCTTTATT
CTATATTATTTTTAAAATTCATCTGTGTTGCTGAATAAAATAGCTCATGTGGTCTTTTCTTTTATTTATTTATTTATTTATTTATTT
TTCTTTTTGAGATTCTTTTATGGGTGTGCTGTCCAGCTAGACTCAAATCCCAGGACTGTTTTAGAAAGTTTTAATCAAAATCCGGGA
TTTCTACCCTGCCTTTGATCATTCAGTTCCCAGATAAAAGACACACTTTTATTATTTACAACAAGCTTTAAACAACACAAGAGCTGG
GCAGATATCTGCCTTCCCTGCTATAAGAATCTACTTTCCTATTTATAACCCCGAGTTATTACTTACTGTGCTTTATCTGGGCTGCTC
CCAGCTTCAGTTGGCCAGCCTTATAGCTGTTTTCTTGACTCCTAACCCATAGTGTCTTTCTCTTCTCTTTCTCCTTCACTTCTTCTT
CCCATCAGAGGTTCTCCTCCAACCCCCAAGCCTTGGAACCCTAAACTCCACCTGAGTCTCTTCTGCCCAGCTGTTGGCTGTCAGCAT
CTATATTTACC2ATCAGAAATAACTTGGGGATAAGGTCACATTAGCATCACTTGGGTCTGAATGTGGACTCCTCTGGGTCAACCAGG
TCTTGAGGAACTAGCAGTATTAGCATTAGAATACAAGCAACATCAGACCAACCCCTACACAGGACTCAAATGATTCTCCTGTCTCAG
CTTGTTAACAGGATTGCTATAGGCATACATCATTTTCCTACATTTACTTAAGTTTATTATTGATATATATCATGTAGTTGTATGTAT
AAAGTACTTTCACTTACCTGTTCTGTCATTGATAGCTATTTAATTTGCTCCCATATTTTTGTGTTTATTGCTGGTTGTTGTCATGGC
AGCTTTTCTTTGTGCACATGATCAAAATGATTCTGTTGCTTTGGGACCTTTGACAAGGGAAAGCAAAGTTGCTCAACTCTTAACGGC
TGGGAAATGGGGACGGGGGTTGGGGAACTTTCCTCAGTATCTTCTGTGGCCTTGAATGGCATACCTTAGGGACCTAAATTCTCTTTA
TTATGCCCTGCCTTTTAAAGGTTTCACTGCCTTCCAATAATGCCAATGTACTAGCCCTTTAACATGTTGTCATTGGGAATACAAATT
TTAGTACGAGACATTCCAGATGCCTGCCTGTTTGCTCTGGCACCCATCACCATGTCCCTTGCCACAACCCCATGTAGGCATTCTGCT
TTGTGATAGGCTGTTTTTTGGCTGCCAGTGAAAGAACCCATTTAAGCTAGATTAGAGTGTATTAAAGGCAGGTTTATTGGGAAGCTG
CATCCAGAGTGGGAGAAGGAGAAGGGGAGAGAACATGCAAAATGTCTAGATTATATAGAGAGGAGCCTCTAGGAGAAGGGCAGCCCA
GCTCCTGGGTTGAAAAATTCAAGGTTGTGGGCAGGGTATGTCAGGTAGGGACTGAGGGATGCTGGGAGAACCTAGAGGCCAGGTCTG
CTGTGCTATGTAAAATATGCACCTTAGTCTCTGTTCCAGGGTCCAGAACCAAACATCTCAGTCTTTTGTTGCTTATAAAAGAATAAG
AGACTCCATAATCATACATGACTGCTTCCTGCTGACACTGGGGTGCAGTTGTGAATGTGAGAAAATGCTGGAATGTGGGGCAAATCC
AGGAAGAGCTGATTGTCTCACTGCCGCCCAGGTTATGGGACCATCTCAGGGTAGGAGTGGGTAGGCCCAGTTGAAACAGTCTGTGAG
GCTGGGGCAGAGCACCTGTGAGTAACTGTTTTGGAGCTCTCGTGGATGCAGATTCTGAGAGAACACCTGGATGTTGGAGACAGAAGA
TAAAGTTTAGGGAGAAAAACTTCTGGGGGGGGGGTTATACGTTTAAAACTCTTAGGCTCATGGTTGTGGGAGTTGGAGAAGGGGTGG
GGTGGGGGCAGGACCAGGGATAGGGCCGGTGGAGAAGGAAACAGGCTGTTAATTGACAGTACTTATCTGGAGTCCATTTGGCTATGG
AAGGTGGATTTGAGTGGATTCCCTGAAGCTTACAAGAATCTTTTTAAGTTTACAAAAAGATAAATCCTCTACAACTTCATTTATCCA
CTTCAATACCCCTTCCTAGACCTTCAAACAGTTTCTTAGAAGCCTTATATATCCTCAGATCTTACGTAAATGTTTCCTATCCAGTTA
TTCACCATGAGACATGGATGGTTGATCACTAGATTTAAACCCCTCAGAAATCTGAGAATGGTGCATCTAAATGTCCTATGTGTCAGT
TAGAATGACAGAGTTACCCATTACCCATTATACCACTAGGTTCCTTTTACGTCGATGGCTTTGTTGAGGACACAGGTCCAGGGCAGG
ATTCATCTTAGGCTGCCAGGCCCAAAAAATCTGGAAGGCCTTCCTGTGGAGGAGAAACTTGGGAAACTGACCTTGCTTTGACTTAGC
CAACATGAGACATGTCTGGTGTCCCTCTTTGTACACAGTTCAGGTCAGACCTCAGCAGTTTAGGCAATGGAGCAGCCTTTCTCAATG
GTTATTGTACAATTCAGGTGGAGTCCTCTTGTGCCTGGATCTTCTTGGAGACCTTGAGGGTGTCACTACTAGGATTGGGGAGTCTCT
ATCATTGTAAGCATTAAGTTTAAGGTTTTAAATGTCATATTCAGCAGGTCTCTGACAGGTTTGAGGCCGGTACCTACTAAGCATATC
TGCGTTAGTCAGTCTATGTCTGTTTCTAGTTATCTGTACACCTAGCAAACATAAAAAAGGAGGCTAAATAAAGCTTAAATAATTAAC
TGCATCATTTCTCAACATGACTTTAGTGATGCCCTTAAGAAAGATGGTGGTGAAGCCATAATTCAACCCTCTTTATTACAAAGTCAA
GATGGTGACCAGTTGTGCTGTTTACCCAATGTGTGTACATTGTTAGTTCACCATCACTGTTGTAATCAGTATTGCTGCTGCTGCTGC
TGCTGCTGCTGCTGCTGCTACAGGAAGGAGCGGGGAGAGGTGGGGAAGTCTGAGCTAGGTGGCACCTGGAGCCTTGTGCTCAGTGTG
GTGAAGGGAGAGTGGGAGAACACAGCCTCCAAAAAGCTGGACACAGGGAATTCAGACCAATTTGAATAAATTTTTTTTTTTAGATCC
CATGGCACCCATCATCCCAGCGTTCACTGGGAGATCCCAACACTATTGAATTAGCATCCCAATTCATAGTCTGAGATCAAATGGTTT
ATGCAGAAGTGTAGTAACCTCAGATAAGGCATGTCTGTATCAAAGGTTCCCCCAAAACAAAGACCTGGCCTTTGTTCAGATGGGGGG
AACTCTAACGTAGCTGTGACTCAGCAGAGAAAGCTCCGAGGATCCTAGACCCAGATAGGAAATAGCTGGCGATGGGTAGCCACAGAG
TCAGAAGAAATGATGTATTTCAGTGGAGAGGGCTAGAAGAACAGATTGGTTCCCAGATGGCCAGGAAATACCAGGTCCTTCGAACTG
GGGACTCAACTTCTCTCCAGTTCAGCGACATCTGATAGGTTATTTTTCCACCACCGGTGAAATGAGCCAATTTAAGCCAGATTAGAT
TAGATTAAAGGCTGGTTTATTGGGAAGCTGCTCTCAGGTGGGTGAGTTCACTGGCCCGGAGGACCAAGGCCAGGGAAATCGCCATAA
GGACAAGGGAAGAGAGGGACAGGAGGGGAGGGAGAGAGAAAGAGAAGCTGCACACACAGAGAGGGAAGAGAAGAGGAGCCTCTGGGG
AAGGGCAGCCCAGCTCCTGGCCTAGAAAGTTCAGGGTTTGGGGCAGGGTATGCCAGGTAGGGACTGAGGGATGCTAGGAGAACCTGG
AAGCCAGATCTGCTTTGGTATGTAAAATTTGTATCTCCACCCCCGATCCTGGGGTCAGAAATGAAACACTTCGTAGGTGTACGTGGC
TAAATTGGCCATTATTAGAAAGACTTGGTTTTACTATTTTTTTTTTAAATACCGTTACAAATAGTTTTGGTAGACCAACATTTTGGC
ATGTGCTTATAATCTAAGTAATCTCAGTATTTGGAAGGCTGAAACCGGAGGAGAGCTGTGAGCCCAAGTTATTCTAGACAGAAGTCC
AAGACCGTGTGTGTAGGTTAAAATAGCCCGAAGATGTCATTCTGTCCTTCCACCATGTGAGTCCCAGGTGATGAGCACCCTTACCCG
CTGAGCTGTCTCTCAGGTCCTGATCCCATTTCTCCTGTTTTCTTCCTTTGAGCAGGTGGTGCTGTGTATCTCAGTTGATGTGTCTCA
AGACTATAACCAAGTGGAGAATGTCATAAAGCAGGTAAGAGGAGAGGTGCTTTCAGTTTCACTGCCTGACTCTGCTCAGCTCTCGCC
AGGAGTTGACAGCTCTTCTCTATAGGCTTAGTGGTTCCATGGACTTGATTGACCTTATCTTTCTGAATGATGAATCTTCCATTCTCT
GATGTAGTGTTTGGAATGATATGTAATGTTATGGAATATTCCTCCAGGCACAAGAGAAGCTGGGTCCTGTGGACATGCTGGTCAACT
GTGCAGGCACCTCTATGTCAGGAAAGTTTGAAGAGCTTGAAGTTAGTAGTTTTGAGGTGAGTCACAGTGGTATCAAGATGATGTCTT
CTATTTTAAAACAGCCCTGCCAAGCCAAGAACTCAGCTACTAATGTACTTCAACACTGAGGAAATGGGCATTAGGTGTGAACTCTTA
AGTGCAACGCCTTTTGACCTTGGCAATACGTCATTCTTGTCTTTAGTGTCCTCTTTCACCAGGGTGATAGGGACTCTGAAGTTAAAC
TGCTCTGCTATCTATCTTATAGGAGGTGTGACTGCTGGACCAGTGTCAGAACTATCTTCTCACAGGCTGTAGTCAACCCCATATACT
CAGAGATACACACAAGGCCTACAGGAGGATCAGAGGGTGGTAAAAGGTAGAATGGTAGGACCTGAGTGTCTGAGTTTGTCTGAGACG
CCCTGTCACTGTGCACATCACGCTGGCCCAGCCTTCTACCTCAGCTTCCTGAGCACTGGGATCACACATCTCTGTCACTGTGCCTAG
TGTCACCGTGCCTAGTGTCACCGTGCCTAGTGTCACTACACCTGGCACAATTGTTTGTTTTACAGTGAATAATTCAGAGGCACTTAG
CAAATTGTGATGTTGTGTAACTATCTAATTCTAAAACATTTTCATCTCTCCAAAAGAATACCCTATACTTTAGTCAGTATTTCTCAT
TAACCCAAACCCTTATTCTAGCCAATGGCTACCCCTAATCTGTGTCTGTCTTGCTGAATAATGGAGTCGTGTGCAGCGTGCGTGCCC
TCCTGTGCCTGGCTTTCTCCCATGCTGAACGCCCTAAAGGTTCCTCCTTCACACTGACGTGTACACCGGTGCTTTGTTTTTCTGAGT
TGGTAACAGTCCAATTGTGTGAAGAGACTGCAGTGTGCTTGCTTGCTTATCACCCCTGAATATTTGGACCATTCTTACCCACCCGGA
CCAGCCCAGAAGCTCCAGGTATCATTTGAGCCCTTGGAGGTTGTGATGCACAACCCTGTGTTGTAGTTTCCCTGTCACCAGTCAACA
CTCAGGTTTCCATGTCTTGCCAAGTGTGGTACAACTTGTAGTGTGTTCTCCAGCTTTAGGCTGTTGTTGCTTTTCTTTTCCCTCTGT
TGTGTTCTCTCGGGGCTCTTAGCTGGATGAAGTGGCTCATGCGCATAATCCCAGCTCTTGGGAAACTGAGGCAGGAGACTAAGGTTG
AGTTTAAGGCCAGCCTGAGCTATGAAGTAAGTTTCAGGCCCTCGTGAGATACAATATGAGACTCTATCTCCAAAACAGAGAGACAAA
TGTACTCCTTACTGGTGTTTTATTGAGTTCTCTCAAAAGAACTGGGGTAAGCATGGAGTTTTGTTCATTCACAGGTTCTTTTGGGGG
GTTCTTACTACTAAAGCAATGTCATGAGGGGCTGGAGAGATGGCTCAGCGGCTGACAGTGTTTGCTGCTCTTGTAGAGGACTGAGGT
TCAGTTCCCAACACTCTGAGAGGTCACATCTGTAATTCCAGTACCAAAGGATCCAGTGCCAGCCAGGCACACACATACATGCACGCA
AGACACTCAAATGCACATAATAAAAATCAAAGTTTTAAAGGCTAAAACAAGGCAGTGATATTAAAACATTCTAGGGGAAAACAAAAA
CAAAAAACAAGCAAGAATCCTGGCTTCCAAACACAATCAATATTTTCAGTTCTCTTTGCTTTTACAAAATAGAGTGGTAAGAATTAC
TATCTGCACATATCCTGTCTTCTACTTTTTGAAAACTCATAGCATAAATTTTTTCCCTCACTCTTGTGTTTTCAGTTCTCTTTCCTT
AAAGTTTCTTAGTATTCTGCCACACTGCTATCATGGTTGTGTATGCTCTGACTGGGCTGTGTAGCTTCTCAGGGTAAGGAGAATAGC
CTTCCGGTATATAAACCCGTCACTGTGTGGCCTTCCGGTGTATAAACCCATCACTGTGTGCCCTTCCGGTGTATAAACCCATCACTG
TGTGCCCTTCCGGTGTATAAACCCGTCAGTCACTGTGCATCCTCAGGTTCTCAGCGTGAACGTGGGGTTCTGGGATGAGAAGCTGGG
ACTGTCTCTGCGGGCACTTTCCAGCAAATACCGAGTTCTTACTGTGCAGGGCAGCAGGATCTGTGCTTGTACTTAGCTTGTGTTTTC
CCGTGTAATCCCTGAAGCATCCTGAGGTCATGCTGAGATTAACCCCATTTTCACCTGAGGAAATGCCCAGCTGAGAAAAGTCTCACA
AGATCAGGGGTGGGTAGTGGCATGAAGGCACAGCTCTACTGTTAACTGGTCCCACGTTCTCTGTATAGTCCTTGTCTAGCTTAGAAC
TAACAGGGCCAAAGAAATATTTTGAAGCTTTCTCCTAAAACTGTCCTTCAAGAATTATTACCTATAAGAACTTAAAAAGATAGCTTA
GGGATTAAGAGTCCTGGCCACTCTGGCAGAGGACCCAGGTTTAGTCACCAGCAACTTACAGCTGTCTTTAATTCCAGTTCAGAAGAT
CCAGCGCCCCATTCTGGCCTCTGGGCACTGCACACACATGGTACACAGAAATATTTTCAGGCAAAACCCTATACACATAAAATATCG
TTATAATGCATTACCTACAGTAAGTACTTTTCTAGTAGAACACTGATTTTGACCATTTGTGTGACCATTTGTGCATGGTTGGAGCAG
AGGTTAATGTTGGCTGTTCGTTCTCGTTCGTTTTCTCTCTCTCTCTCTCTCTCTCTCTCTCTCTCTCTCTCTCTGTCACTATTTTGG
TTACACTGGCTGAGACTGTCCAGTAAGCTGCTGGGATCTGCCTGTTTCTGCCTTCTTGCTCCCGGTGCCTGGGTTAGAGGTGTGTTC
CCTTATGCCCCGCAGAGTTTTTGATGGGGATCCAAACTCAAGTCCATATGCTTGCACACAAAGGACTTTCCTAACTCAACCTTGTGC
CTAGCCCTAATTTTGACATTTATATCAAAAATGGAACTGCTGTCACTATGAGAATTGTCTTCAAACTAGTCTCCAGAGGCTAGGTCC
TGAGTGTCATAGTATCTACTACGTTTGTTCACTTCCCCATACAATATTCAGCAAATCTCCTGCACCCTGGCTCCCAGTCCCCTGGGT
TTTTTTTTTTTTAATTTAGTTGTTATTCTATTGCTGTGAAGAGACACTGACCAATGCAACTCTTATAAAGGAAAGCATTTAATTGGA
GCATGCTTACAGGTTCAGAGGCTCAGTCCATTATGATCGTGGTGGGAAGCAGACAGGCATAATGCTAGAGCAGTAGCTGAGAGCTTT
ATATCCTGGTCCACAGGCAAGAGAGAGAAGAGGGAGGGGAGGAAGGAGGGAGGGAGAGGGAGAGAGAGAGAACGAGAACGAGAGAAT
GAGAACACGAACACAGACAGACACTGGGTCTACTGTGGACTTTTGAAACCTCAGCATACCCCCAGAAACACACCTTCTCCAACAAGG
CCCCCCTACCTAATCCTTCCAAACTATTAAACCAAATAGGAACCATGCCTACAAATATATATGCAAACTACCATAGGGTCTCTCTTA
AATAAGTTAGTCCTGGGAAGAGACAAACTCCCCTTAGCAGGGCTTAGGCTTTTGTCCTTGTTCGTCCCACCTCATCTGGCCTCTTTG
TTTCTTGCAGAAATTAATGAGCATAAATTACCTGGGCAGCGTGTACCCCAGCAGGGCAGTAATCACTACCATGAAGGAGCGACGGGT
GGGCAGGATCGTGTTTGTGTCCTCTCAGGCAGGACAGCTGGGACTGTTTGGTTTCACGGCCTACTCTTCATCCAAGTTTGCCATAAG
AGGATTGGCAGAAGCTCTGCAGATGGAGGTAAAAAATACATTTATTTCTGTATTTCTTCTTCTTAGATCAGCAGGGAGCTCTGTCAT
TTTTTAGCTCCTTAGCCTACATCACTTTGTAAGCTAAAGGCTTACAGAGGTGATATCTGTAAAAACAAAACAAAACTAACTCAGTTG
TAAGATCTGTGTTTTAATACTTCTTATTGTATAAAATTATACTTAGGACAGCGGCAAAATGACTCTGAGGGTGGAGGGATTGTCTAC
TGTATAAAGTACTTGAAATCCCTTCCTGGTGTTGGTCTGCTTCTGCTTAAGTGAACAGGGATATCATTATAGACAACTTTCTGAAAG
CTCTGGATGTGGTTCTGGGGAAAATGCTTGTTTTACAGGTTGGGTAACAGAGTTTGACTCTCCAGAACTCACATACATGTTGGGCCA
GTGTAGCAGTCTGCCTGAAATTCTAGTGCTCAGAAGGTGAAGATGGGAGCCCAAAGCCGCGTGGCTAACGAGCTAGACCAGTCCTCT
CAGTATGAGTCAAATTTGCGAGGCTTACGTCAAAGAATAAGGTAGAGGATCATTAAGACAAACTCAATATTAGGCTTAGGCCTCTGC
ACACACATGCTCAGTTAAGCTGTGAAGTTGCAAGAGAGGATCCTGGTGATTCTGTGCAAGGATGCTTTGCAAATAACCATGTTCGTG
TGTGCATAGGTCTGTATGTGTGAAATATGTGCACAAACTTCTTCATACCACTTTCTGGAAAGGGGAATTCAGCTTTTAGTGAATTCA
GTGAGTTACTTAGAGTCACATAGTCACTTGGTGACAGGAATAACAAGGCCTAGCCCCGTAACTGCAAGCTCCACTGGTCTTTTGCAG
CAGAGGATGTATCTGTAATTTGGAATGTGCATGTGATGTGCATCTGGTAGCTGCTTTATCTGGTTTGGGATTTTGGGCTTAGAATCC
ATTCGGGTGCAGAGTTTGAGAGATAAAAATGTATGCCCATTAAGGCCTTTCCTGAAGTCTGACAGTATGATCCACAAATTAAGGGCA
AAGTGTATCTCTGGGAGCCCTGGTTTTCATTGCAGAGGGAACAGTGACCCAGTGGCTGCATCAGAGTCGCTCAGCTTCTTGATGTCA
AGAGTGATCTTTCACACTGCCCAAAGCTGACTGGGCTAAGTCGCCTTGACTTCTGAGCATTAAAATTGTTGTTACTTAAATAAACAA
ACACACATACCATCTAAAAGACTGCTATTTTATTTGGATTAAGTAAATTCAGACTTTTGAATCCAACTTGTTTGTAGCAAAATTTGC
CTCAAACCAAGATTTTCTTATCAACTAATAATTGTTTATTGAAATCTAGATAATTTGGTCAGTTTTTTATGAGTTAAACCAACATAG
TTGCTTAAATGCTTGATGCTTTGGGTAAACATTTTGGTTTAATTAACTTTTTAAGTTTTTCTAGTTACTATCCTGCTTCCTCCGACA
AGGTATAAACTCTGAAAATGTGGGCACAGTGTGTACTTGAATGCTTTGTTGATATTTTGGATTTGATTTTCTTTTTTCTTCTTTGGT
CTTTTAAATACAAAATTAATATTTAAAGAATTTTTTTTCAAAAAGTTGATCTCTTCGAAAGTGTATTTCTGAAGTCAGTTATAGCCA
GAGATTCAAGAATCAACCATAACAAAACAGGAATCCCCAGCCAGGATTCCTCCTGTTTTCTCACCCAGACCATGCCTGGCCCCTCCC
CTCATCTCTTCCCACCATGGTAGACCAGTAAGCTGGAGCTGGGATTCTTTAGTTTGATATAAGCAGCTGAGGTTCCTTCTCCAGCCC
CAGTGTTCTTGTTTTAATAGAACAGCTCTTAAGTGGCACTGTTTTTTTATGATAAACTCATCCTTCGCAAGCCATCCATTGTCTTAC
CTGTGCGAGGCTGGTGCCAAGTAGAAGGGAGAGTAAGAGGACAGGTGAAGAAAGGCAGAGCAGGAGAGTAGAAAACGGCCCTTTCCC
AGGGCCAGGGAGAAACAGGTCAGCGAGGCATGAGTCAGTGAGATGAGTGTAACACTCGTCGTAACCCTCGTCGTAACCTAGAACAGG
CGAATGTAAGCTAAGCCGTCCCTTTTTGGTTGGGTTATAGTGTTAACAGTTAGAGAGGTGTAAGAATAATTTTGTATGTCCTGGTTT
TTGCAAATTATGTGAACAAGAGTATCAGGAGACCAGGAACTGGGTTATGGACAGAGAGTTGTCCGTTTAGTTCATTCCTGTACGGAA
GTCTATGTTGGCATACAGTAGTAATTTAGAAAGTGTTTACTGGAAGCATCCACCCAGATGCTAGCAGAATAATTGTGCTTGATGATT
GCAAAAACAGGGTTGGCTCCAAAGGTGATGGACTGGTTCCTCTCCACCTTGCTGTTTCCAGTCCCTCCCTGTGAGAGAGCGGTTGCT
AACCCAGTCATGACCTTGAGTTTGAGAGACTTTGAAACATGGTTTTGATGATCATAAACGGCACAGCCCCCACTGTGGGGCAGCGCA
GTGGGGGAGGCAGTTCAGAGTGGGCCAGAGAAGCCAGCCTGTGTGACTTGCGTAGTTAACACTCAGGCACTTTTTTTTTTAAAGCCA
TTTTTCTCTGGTCCAATACAGGACCAGACAAGACTACAAGAGACATTTTATCAATTTTCACCATCTCAAAAGCAAGTGTGAGAGAAG
TCACTTGATCTTTGTTCCAGCTGGCTCACCTGATTGGCCTGGGAGACAGCAAGCACTCCCTGGCAAGGCCAAGCCATGAACCCTCAT
GACTTGGATGCCTGGAATGGAACCAGCTTTCATTCTTGTGATTACTTGGATGGGTGCACAGTACCTACCTACTGCTTTGTTCTCTTG
ACCCATGTTGCTGTCCCGAGCCTTCATAGCTGTGTCACTAAAAGAGTCTGGGAGCTAAGGGGCTGGCGCCCTGGTAGACGGGTGGGG
CTGAGAAAACCTGTCGGATTTCTTCTTTAAGATTCTCAACACTAGACTTGTTGCCGTCCCTCCCAAACACCCAGATGTCTTTATGAG
GACAGAGACACAAGCCAGCAGTACATAAAAAAGCATGCCAGAGTGGACTTGGTGGTCTGAGCCTTTGGATCGCTGATGTCCAGGCAA
GAAAGGAACAGGCGACAGCGACCGGACTGCTGTTGGACAGTTTTGGGAGACTGACCCTTGAAAGAAGGGCCAGTGGGGTTGTTGGGG
TGTCTTCATCAGTAGAGGAGTTGACTGGGCAGAGAAAGTGGGAGGCAGACAGTGTTGGAAGAACTAGAGGTTATAATAGGCCTAGTA
CCTATAAAGAAAGCCAGGGAGAACTGGGCATAGTGGTGCACACTTGTATCCCAGGTCTTCGGGAGACCATGGCAGACAGATCACCAG
GTCAGACTAGCCTGGGCTAGAGAGATCTTACTGATAAAAAGGGCAGAGGGAAGAGCTTCTCTGTATAAATAGTGGTAGGGGCTGTTT
ACAGGGGAGTTGCTTGGCTGGTCATACTTTGGTGGTCTACCCAGGATGAAGATGCTTTCTTCCTGATGCTATGTGGGTGGTTATGCA
GGGACTTCTACAGGGATGGATAGAGTTTCCAAATCTGGATTCAGACACAGTAATGTGCATCCGGGTAGGCACTGAGAGACGGTGTTT
TGCAATTGAAATGAACATTGCTGTATTATAGCAGCCTGAAAGGCATAGACCTGCCTTCTGCCTTGAGCTGTGCAGTTCGTGGGCAGA
GTCTGTGATGAAGGAGCATACTGTCATTCCACAGCATATGCAGTTCTCCCTGGGCAGCTCTGAGCCTCTGCTTGGTCAGCCGCAGGA
AGAACTCTACTAGGAGGGGGCTTGACACACTTTAGAGCCAGCCACTTTCTGACATAGGTCAGGGGTACTCTTGCTACTGTTTCTAAG
TCATCTTCATGGAGAGGGGTCCCCAGGACATTTGGTGCCTGTTTACTGATAGCCACTTCATGAACCTGACAAGCAACACCTTCTAAG
GCAGTGCTCTCAGCCTTCCTAGTGCTGTGACCCATTAATACACCTTGTGTTGTGGTGACCCCCAACCATAAGGTGGTTAGTTGCTAT
TTCATAACTGTAATGTTGCTACTATTATGAATCAGTGTAAATATCTGATATATAACACCCCCCCCCAAAAGGGCTGTGGCCCACAGG
TTGGAAGCACTGTTCTAAGAATCAAGATAGCTGAGACAAGTTACAGGATGACTACAAAGTGCTCTGTTCACTGAGAGTGCGTACCTT
GTTTGTGTGACCAGGTGAAGCCGTACAATGTGTACGTCACTGTGGCCTACCCACCAGACACCGACACGCCGGGGCTGGCTGAGGAAA
ACAAAACGAAGGTCAGTATTCCTACATAATGCCCTCTGTGAGGAGTGTGTGATGGCGTGTGTGTGTGTGTGTGTGTGTGTGTGTGTG
TGTGCTGTGTGAAGCGTTTTGTGTGTGTAATATGTGTTGTGTGTGTGAAATACATGTGTGTGATATGCATGTTGCGTGTGAAATGTG
TGTATGATCTTCTGTGTGTGCTGTGGGGTGTGATGCATATATTTACACAGATTCATAATAGTAGCAACATTACAGTTATGAAATAAC
AACTAACAACCTTATGGGTGGGGGTCACCTCAACACGATGTGTATTGTCTGTGTTATGTATTGTATGTGTGTGATGGATGATGTGTT
TGATACAGTGTTAAGTGTTCTGAGTGAGGTGTGTATGTGATGTGTGTGTGTGTGCACGTGTGCATGGTGGTGTGTATATAGTTTGTA
TTTGTGACTGGAGGAAGTTGTGCATGTTTGGGTGTGTGTGTGTAGTGTGATGTGTATATGTGATATATGGGGGTGTTATACATATTT
GGGTTTGTGTGGTATGTATGTACATGTTTGGGTGTATATGTGCGTGTGCATGTGTGTGTGTGTGTTTGGGAGTGTGCAGGTGTGTAT
GGAGACCAGAGAACACCCTTGGGTATTCTTTTTCAGGTGGTGTCCTTGGTCTTGAGACAGGGTCTCCTACTGTCCTGGAACTATCAA
GTAGGCCCGGGACTTCTCTGGTCTCTACCTCCTCCTGGCTGGCTTTGCATGTGCGGGTGCTCAGAGGCCAGACAGGCACTTTATCTC
CCTAGCTCTGTTCTGAATCTTCAGCGTAATTTTTATGAATGTATCCTACTTTAGGTCCTGGCTGTTTATCAGTTAGTGGGTCTGTTA
GGTGCAGGGTGCTGAGCCAAGACTTGACCTTCCAGGCAGAGCTTCCTCCTCCCTTTTTCTGCTCTCCAGTCCCCTTTTCTTAATGTT
GAACTTACTTGTTTCCAACTTTTAGCTCAGTATTTTCTCATAGGACGCAGTTGGCTGCCTAATGCTACCTGTGGCCTTATCACTCCC
ACTTCTGACCCCTGACCCCTAGAGGGAGCCATTTCACCTATGTGTCTAAACCAATCCATACATACATACCTACATACATACACAGTG
AATGCACCTACATGCATGCATAACATGCATACATATATGCACATACATTCATATGCATCTACGTACAAACTCGTGTTTTAACTAGGG
AAGCTAAAATTTGAGCCTCAGGTCTACTTTGTTCTGCCTGTGTGACCTCTGTTGGAGTCAGATTCTGTGTTTATGAGTGTTGATAAA
GTTGAACCTCTATAATGGCATTTCCTTTCCTGTGTGTCTTTGTTTAACTGAGAGCATTTGTAGGTCATTCACTGGGGAGAACTGGAC
GTCTTTTGAGCATCTGAACCACTGACCTTTCTGTTACTTTCATTCTTCTTCCTCCAAGCCCCTTGTCATGGGGCTCCACCTTCTGCC
AGGACACAGGCCCTGGGGGTGACATCCTCTGCACTCCCCTGTGCTGCGCTTCCCTTCTCTGTCCCTTGCTCGGGTCTGTGCCTTCCA
CTGCTGTCATTTCTCTGCTCACCTTGGTGGCCCTTCAGTCATTCCAGAGAAAGGCACGTGAGAGGCACCATTTGTTTAGAGTCTGGT
TCACAAGAAGGCATCTTCCTTTTCCTATTTTTCGTTGCTGTGTGTGGTAGGGTGTTTTACTGGGTGACAGTTGAAAATGACGGTCCT
CACGGCTTTGCGGGCATTTCTTACTGAGTTCTAGCTTCTAGGGTTTCCTGGGGACCCCACTGACCTGAGATGCCTGAGAAAAGCAAC
TCGTGGCTTGTGGTTCAGAGACACCCGTCCATCAGGACAGGGAGAACTGGCCCGGCAGATCAGTGCTCCTTACAGGAAGGAGAAAAA
GGGGGGACAGCAGAGAGCCAGCTGAGCCATAGCCTCAGCAACACATTCCTCCAGCTGGGCTTCAGCCCCTACCTTTCCCCACCTTCC
ACTAATGCTCAGGGGCCAAGCAAGCATCCGTGACTGAGCAGCTTCCTCATACACATGGGAGGTGTGTACTCCTGGTGGGGTTTAGTT
GTAGACACTCTTTTCCTTTTTATCTTTTAGAATGCCGTTTGGTGTGAAGTGTTTTATATGCTAGATACTCAGGACCGCCCCTTTGTT
CTCTCATGCACTCAGGTCTGGAACATTGAGGTCATTGTTTCCCATCCAGAACACTTGGAAGCATTCTTTGTGCAGTTGTCGCTCAGG
AGCCCCACTTTCCGGCTGACTTCACCACCTCATCTCTTACTCACTATTGGTTTCTTCTTCAACTTTATTTAATTTAACTTCAATTTT
TATTTCTACTGATTTTTTTAAGCTTAGATCTATCTGTCATGCATTTGTCTTTAGTTACTGTTCTGTTGTTGTGAAGAGAAACCAAGA
CCAATATAACATAAAAGAGAGCATCTACTTGGCTCTGTCTTACAGTTTCATAGGGCTAACCATTATCATCATGGCAGGGATCATGGT
GGCTCTCAGACAGATGTGGTGCTGAAGAAGGAAATCAGAGCTAAATCCTGATCCACAGGCCACAGGCAGGTAGACAGACAGACAGAC
ACACACACACACTGCACCTGGTGTGGACTTTTGAAGCCTCCACCCCGTTCCCACTGAGTTTTCTTCTATTTCTAAGGAGCTTCAGTC
AGAATGTTGCTTGGTTGTATTTTTGTTCTGTTTCTTTTCTTGGTTTTGAGTCTAGGTGTTCTCGACTCTGCTCATGTTGGTGATTGC
ATTCAGCAAGGCAGTGGAGCACAGGTTGTAGAGCCATGTAAAGCCCTTCCGCCACCTTCTGCTTTACATATGGAGGATGGGGTGGCA
GTGGCGCCTTCGTTTCCTTAGCGTCTCGTGTGTGGACAGATGGCATGGGACTGGTCATTTCCGCCCCAGTGGCATCTATCTCCCTTG
CATGTGTGCAGGTCATACAGAATGACTGGGTAGTCCTGAGCTGAGAGTAGGCTCTATTCATCTGGTTCCCCATCTTTCCCTCTGCCA
TGACACAAACAAACAAAACAGATAAACAGGTAAAGTGGGGCTGAAGGAAACCCACGAAAGCTTACCACCTCACACTGAAGGTAACCT
AGTTTTAGACTTAATTATTTTATGTGGTTTTTCCTCTTAAAGTGTATGTGGATCTCTGTAGCATAAATTTGGGGTGTTTATGTAGTC
TATATTCGGTTTTTGTTTGCCAAAAAAAAATGGGTTATTTATGTAGAAACAACCTCTTCATTTTCTTTTGAGTTCAGTCAATTTTAT
AATTTCTTTTTGCTTTAAAAACATAACTTGGAACCAAACCTCCTGCTTCAAACCTGCTAAGCAAGCATTAATATTTGCTGCATACTC
TTTAAGTCTGAAGCCAGTGAAGTAGTGACTTCCCTGAGATTTGGTTGTTTTGTTTTTGTTTAGCCTACAGTAAAATTTTAGACAGAT
TTCTAGTGACTGGAACCTGCATTGATCTGTGGGCCCAATAGGGGGCAGCAGACACTCTTCTAACTCAGTGCATCTTGCCTGTCCTTA
TCCACCTGGTGTGGAGGGAGAGCAAATCGGTGAGATGTGTGTCCTCTCAGATTCTAAAAAAAAGAAAACCTGACTTTTGTGAAACAT
GAGCTATGTTTCTATAATCACAGTATTCAGGAGGTCGCAAGCCTGCTTGGGCTACATAGTCAGACCCTGACTCACAAAACCAGACAT
GGAGGAAAAAGTGTATTCTAATCTTTCCCCTCGAGCCTACATAGTCACATTTAGTCTCCCGTGGCTTAAGGCTTCAGTTCGAAACTT
TTCTTTTGAAGAATGTTTGGATGTGTGTGTAAAATGCCACTCAGAAAAGCCAATGGGAACAGACAAAAACGTTGCCGCTCACAAGTC
AATTAGGACAGAAAGTGTGAATACAGAAGTAGCTAGAACGTGAATTAATGCAGAAATGAAGGAGAGGAGAGAGGGAGAGAAGGAGGG
GGGAGGAGAGAGAGAAACAGACACAAGAAGCCCGGAATGAGGAAAGGTGTTCTGAGCTAAGTGCGAGCCGGACAGACCTTTATCTGT
CTCTTGAGGAAGAGAAGCCTAGGATTTGGGAATTGGGCCCTGAGTCCTGTAACTACCTCTCAGCCAAACTAGGCAGGAGGCAGTGTT
GCTACTCAAATGGCTAGTGTCTTCGCCTCCTCTGCTCGTCCTTCAGCAAATAGATACAGTTCTTCAATGGGGGCTAGGCCCAGACTG
TCCTCAATCTGTCCCCAGCATCATTCATCTGCTCAATGGGGACTCTATTCAGAATGGAGGCACGGTCCTACGATGGATCACGTTTGC
TCAGTCACAGGGACGAGCCTCTATGCACATACTGTCTGGCAGTTGTCTGTCCAGTAAGAGAAGGGGCCCTTCCTCACTGTGACTGAC
AGACTGATCTACTAGCTCCGGTTCTGGAAGGCCTCACTAGAGCCGTGCTTGCACCACCCAGAACAGCAAGTACCTCCTTAGCCCTGC
CAATGGAGATAAAATCCCACACGCAGAGACTGACTTAGAATCTAGCTTGATCTTTGTCTTGAACAAACGTATTTGTTCGAAGGTAGA
AAAATGATAATTATTTGTGATCCATCTATAAGATGTTTATTTTAATTCAGTGGAATTATTTTAATTTGTATATTTCTCCCACTTGCA
GCCCCTGGAGACCCGGCTTATCTCAGAGACCACAGCTATTTGCAAACCAGAGCAGGTGGCCAAACAAATTGTCAAAGATGCCATAGT
AAGTAAACGCCCTTGTGTGGCTCTCTGTACTCCGTGTATTTATACTGTGTTCCTGAAAGGCACATAAGGACTGACTTCAGAGCAGAG
CGTTCGGCTTTGCGTGGGGTTTGTCTGACGTCAGCCTCCACAACCTCCTCCCACTTCCTGTGGATTCAGCATTAACCAGAGGGATAG
CGTCACGGTATTGTAAGAGAGCTGAAGCTCTCGGAACGCAAATGACCACTACGTGAGGAGAGGCAACTTCTTTTAATTTTTATTTGT
AATTTTACTTATTGGGTGGGTGTGAATGTTGCCTTCTTACAAGAAAATTTCTCTCATCTACCACGAGAGTCCAGGGCATTGAACTCG
GGTCATTAGGCTTGGTGTCTGTGCTTACCCACTGAGCCTGGCCTTGTCCTTTACCAGGGTTTCAGACCATCTAAGGTATTTGATTGC
CAGGCTCAGGATGGGTTTTTCTTTCCCTTCCTGTTTCCTGTTACTACTTTTCTTTCTCATGGTTCACAACAAAGCTGCTGGCCCCAC
CGTTCCTCTCGATCTACCTCCTCACACACTTAATTTGTGGGGAACTGGCTTTTGAAACCATATCAAGCATTATATAGAACACTGCAT
ACCACAGTTCTTTTGAATACATTCTGATTAACTCACTTGTAGAACAAGGTTCAATCTGAGGGATGTCCGTAAACTTAGTAACCTACG
TGACTTTGGAAAGTTTGCACCCAGATAACATGGTGCAGCTTCTAAGTAGCAAAAGGGGTGTTGTGCCAAGGTTAGCAGGACACAGTA
ACAGCAGCCTCACCAACAGACCCAAAGACCTGGTGAGACAGCTCAGTGGGTAAAGGGACTTGCCACCAATCCTAATAACCCGAGTTC
TTCAGACTCCAGTAGTGGAAGGAGAGACCTGACGCCCTCATTTGTCCTCTGGCCTCTACATACATGTTGTGTGATACATACATATAC
ACAAGTATGTTGCGCGCGCTCAACTGGCCAGGAAGAACGACGCTGCTACAGGATCCTTCTGCACACGTTTATTCAGTCCTGTTTCTT
CTTTCTCTATATTTCCCTTGTTTATATCTCCCTTGTTTATATCTCTCCCTTGTTTTTATCTCCCTTGTTTTTATATCTCCCCCGAAC
CCTGGGCCTCTCACTCTTTTATACTCTCAGTTCCCATCCACGCACAGCAGGCCACGCCACCTCACCAGGCACGCAGCTTCAGCTAAT
CAGGGCAGCAGGGGCAAATCTCCACCAAATTCGATTCACCTGTATCCTGGTACACCTGCGCAGCACTCAAGATGTTTGTGGCTTATA
TGAGGAAGTCAGGTGCAAGTCATATGACTTAGCTGCAGTCTCTGGCGCCTTTGGGACTGCCGCCACACCCGCTCCCCACAATTCCCC
CTTTTTTCTTTTTTGGCAGAGAGAATGTCTGTAGATAGCCCCCCGCAGCCATGTCCCTTACCCGTCCTTGGGTGACAAACAGCATTG
GTTTGATCCCTGTCTTAGGTTGGTGACATGCCCAGGGACTCTTATCACTGACTACCTCTCTATCATGCCAAGCCACTCCTGGGGAGA
TTGTGTTTTGCTTGCAGCAGGTGCATCAAAAGGCCAGGATGTTAATTAACCTATGGCCAGATCTTAATTGCTGCAAGCAAGCCTCAT
GGGTGAAGGTAGAGGTCTGATCCCCAATGTGCAAGCATAGGGGCCCTACACAAAACCATCCCTTAGGCTCATCACCCAGAGAGGTCT
TGGCCAGCTCCCGTGTCGTTTTTCCTGGGGGAAGGGAACTAGGACACTGAACCTTCATGGATCAGACATACCTTCCACAGGATGCCA
ACAGCAAGCTATCCTCTGTGCAGTGCTTAGCCTCGCACCCCACGGCATAACGCAGCATAATTTCTTTTAGAGTGGCAAACCAAATCT
GAGGAGATTGTCCCGCCTCCACTGCAGCAAAAGCCTACGCTACCATGGCGAAAGTGCGGGCCGCACACTCTGCACCTAACACATGTG
CCAAACACAAAACACACACACACTATAACAAGCATACATCCCAGGGCTCTCATCCCCGCTCATCTTCCCTGAAGCAAGGGATAGATA
GCCAGAGGGGCTATCTCTTTAAGGGAAATTCAGGGATCAAAAAGCGTGGAAAATTTTGAATGTCATGTCGAGCTGGTATCGGCTTCT
GGAATACCGAAAGGATCTCCCATCTCGGCTCCATCCTTTGTGCTTGTGGGATCATCCACCACATCCACAGGGGCAACTGGATCAGGA
GCCTCATTCTTGCAGCGTCTCACCAATCTCTCCAGCACCCAAAGAGGTTCTGTCTGGTCCTGTGGAAACACACAGACCCTCTCGCCC
ATGTCAACACCTGATCCTGTCGATCCTCTCCAAATCTCCGGACAGAAGGTCCACCTACAGGTGGCTGCTGAGGAGGCATAGGGAGGC
GGCACAAAAGCTAATTCGCCTTCCTCCTCCACCTCGGCTCTTTTATTTCGTCCTTCCTGACCACCTGATAACACTGTGTCTCCTTTC
TTTTTCCTTTTTCTTTTTAAGCCCTTCTCCTCTTCCGACATAATTTCTTGTTGCTCTTTAAGGATTTTCTGACCTGTCTTGACTGCC
TCTACACACAGTTTCAAACAGTAACAGAGTCCATATATCAGAACAAGCAAGACCAAAACTATCAGACCTACCCACAAAGGGTCAATG
GGAGAAAAAAGCATAACTACTCAGCGAGGATCTATTGATCACTACACTGAGGTTATCATAGTTCCTTAACTTGTCCCCAAACCGGGA
ACCTTAACTTGTCCCAAAGCCGTGAACCTTTAGTTACCTTGTACCTGCTTCCTGGCAACGTTATATTCGCCTCTATTTTATCGGAGG
TCCTTCCCAAACTCCTGGGTTACTTTGTGCCTACTTCCTGGCAACTTTATGCTCACCTCTATTTTATCCGAGGTCCTTCCCAAACTC
CTGGGTTACTTTGTGCCTACTTCCTGGCAACTTTATGCTCACTTCTATTTTATCCGAGGTCCTTCCCAAACTCCTGGGGTCGCAAGT
TTCACTCACCGTGAACTTACCCTGCGGGCAACCACTGACTGCTGAAAGTTCTGAACTCGGTGGGGGAGTCGGTTCCCCATACGGGCC
ACCAATTGTCGCGCCCACTCTCAACCAGCAAGAACGACACGACCACCAGTCCTTCTAACAGCAGTTTATTCCGTCCTCATCTTTCTT
CTTTCTCTTCATCAGTACCGTTCCCCAGCTGAAGAGTTCTGAATCCACGCCGGATCCTTCTCAACAGTCTGTTTTACGGGAACTTTT
ATTAACCACTCCTTCCCCGTGATGCAGTTCTGAATCCTCCCTGTAGCAGGGGGTCTTCACTCATGCCTGAAGATGTTTCTTGTCCCG
GGTTTCGGCACCAACTTGTTGCGCGCGCTCAACTGGCCAGGAAGAACGACGCTGCTACAGGATCCTTCTGTACACGTTTATTCAGTC
CTGTTTCTTCTTTCTCTATATTTCCCTTGTTTATATCTCCCTTGTTTATATCTCTCCCTTGTTTATATCTCTCCCTTGTTTTTATAT
CTCCCTTGTTTATATCTCCCTTGTTTTTATATCTCCCCCGAACCCTGGGCCTCTCACTCTTTTATACTCTCAGTTCCCATCCACGCA
CAGCAGGCCACGCCACCTCACCAGGCACGCAGCTTCAGCTAATCAGGGCAGCAGGGGCAAATCTCCACCAAATTGGATTCACCTGTA
TCCTGGTACACCTGCGCAGCACTCAAGATGTTTGTGGCTTATATGAGGAAGTCAGGTGCAAGTCATATGACTTAGCTGCAGTCTCTG
GCGCCTTTGGGACTGCCGCCACACCCGCTCCCCACACAAGTAAATAAATATACATTTAAAAAAAAAGAACAGGTCTGCAGAGGTGGC
TTGGTAAGTTCTACAAGCACAGGACCCCACTTCAGAAGCCACATGAGCCTCCACTCCCACCCCTTTCTGGCCTTCTTGGACACGGTA
CCATTACATACATGGTGCACATGCATACACGCAGGCACCCACACATACATATGTTTTAATTTTAAATCTTAAAACAACAACAAAAGG
ACTGAGTTCCTAGCAGTAACATAAAAGCCAGTAAGCCAGTATGGTTGTTCCTGTCTGTAACCCAAGTCCTGTGTGGACAGACCGGCA
GATCCCCAGATCTTGTGAGCCAGCCAACCCATCCAGTCTGTGAGCCTAGCCAGGGTCATTTTGAGACCCGTTCACAAAAGATAAGGT
GTAGAGTGATTGAGGTAGATACCCAACATTGACCTGTGATTCCCCACACATGTGCCCACAGACATGTAGTTTGCCCTGGTTGCCTTT
AGAGACTTGGAGCAGTGGCACAGAGGCATGAAAGGCTAATTTGCTTTTCCCTCAAGTCAGAACAAGCACCCTCAAGGTGCTGCTGCT
GCTGCTGCTGCTGCTGCTGCTGCTGCTGCATTGTCTTAAGATGCTGGTACTTGCCATGCGTGTGCATGGTGTTGGGTTCCATCACCA
GCAGAAGAAGATGGGGAAGAGATTTAGCTAGGAGATAAAGTCATTTGAAATCAAAGAAATTGAGACTGGGGAATTGCCACAGTGATT
AAAAGCACTTGTTCTTGTAAAAGGCTCTAGATTCATTTCCCACCACCACATGGAGGCTCAGCAGTTCCTCAGGTGTCATGTTGTACC
CAGACATGCATGCCATCAACACACACTCACACACACACACACACACACACAGAGAGAGAGAGAGAGAGAATAAAACTTAACTATATT
ATTAACTCCATTTTTAAATAGCTTTATTTGTTCTTTTAATGTATCTTTGTGCCTGAGTGTATGTATCTATGTGCAATGCCTGCAGAG
GCCAGGAGAACACCACATCCCTTGGACTGGATGTATAGGCATTGTCCCCTCTCTTTATGGTTTCTTTTTAAGTGTGTGTGTGTGTGT
GTAACACTCAGAAGACAGATTTGAGGAGTCATTATTTCCTTCACTGGGGTTCTGGGATCAGACTCAAGTGGCAAGAGCTCTCGTCCG
CGGAGTCCTGCCTTTTCTCAGTTTCTCTTCATATGGGGCTCCAGTAGCTTCAGCCTCAATAGCAGCTGAGGGTGACCTTGAACTACT
GACTTCCCACTTCTCAGTGCTGATATAACTACTATGTGTGGCTCAAACATAGCTATTTTCAAACAGAAATTCATCAGGAGGAGTGGC
ATGGTATTTTCACGAGCCTTTACAAAGTCCACACTAGTGGATGCCAGCTGCTTTCTCGTGTCTGCTTCTGTGGCCAATCTGTTAAAT
ACATTCATTTTGAAACATTTGAAGAAAATGTCACTTTAATCAGATACATAGTTGGGAGGGGATAAAGTTTTCACGGGATTTTCAGAT
AATTATGAACATAATGTTCTTTCTGTTTTTATTTTTATATTAACCCCCATCTTGACAATTAGGCGTTTCTTAAAGGTTGGTTGCTGT
ATGAAATTAGAAACCACACACCAGTGAGCTTTTTGTTCTCGGTGACACTAAAATGCATGTCTCGTTTCTATATTGAATGGATCTTTC
ACTCATTCATCATTTGAAAACATGCATTAATCATTTGGAAAATATGGAATCTGTGAGTTATCAAAAGTCACATTCATCAGAGATCAG
GGAAATATCAGGAAGCCAACTGTCTCCTACTGATGGATAAAGTTTTCATAAGTCTTAACTTTTCCCCATGCACATATATAATCTGTG
GTCTTCTTCCTAGCCACAAGTTCAGTTCATTTGTCTGCCCTATCTGTGCCTCAAGCCCAAATTGGAATGTTCTAGCCTCCTGGTAAA
TAGTTGTTAACAGGAAAATAGTTTTGTCAGGAAGCTCAGCTCTGAGGTGTTCCCAGTTAAGCTAAATCACTCGTGGTGAATTTCTTA
TTTGTCACCTGAGAACAGGAGTCCTCTTCCCAGTGTCCTCCAGGAGATGGGCCACAACCAGCCAGATGTCAGATGCTGCTGGTTCCT
GGGTATGATGCTATGTGTACTCTCCTGTGTATATACCTGTGAAATAGCCTAGCTGGGGATGGAGCTCATTGGTAGAGTAATAGTCTA
GTATGTATGAAGCCCTGGTTCCTCCCTAGTACCATATAAACTGAGAACACATGCCTATAATCCCAGCACTTGAGGAGTAGATAAGAG
AGGATTGAAATTCAGGGTCATCCTCAGTTACAAAGTGAGCTCAAGCGCAGCCTGAAATCCTGCCTCAAAAACAAACCCCAAACCTGT
TTAATGAATGGATTAGGCCCCTGGTTCTATTGAGGAGCAAAGAAGTGATACATATCCCACGTGGGGCAGAGCTGGCCAGCCCAAGAT
GCTGGGGTATCATGCAGACAGCATGCAGTTGACCCCTTCTGAACCACTTCTTAAATACAAAGCGTCTTTTGGGCTCTTAAAGACTTT
TTTAGAGTGTGTGTGTGTGTGTGTGTGTGTGTGTTTTCATTGCTGTGGTGGTGGTGGCACTGGCTGCATTAGGGCCACACTCTCAGA
AACTCTCCTTTGTGCTGTGGTGCTTTTGTCCATATGCACAAAGGTCAGTGGGAAAGCAGCTAACGTCTCAGTGTTATTTGACGGTTT
CATTGTCCCTAGAGACCCTGAAAGGGTCAGGAACCCACAGGGATCTGTGGACTTGTACTTTCAGAGCTGGACATACTTTACGTCTCT
GTGTCCCTTTTTAGTTCTGGTATATTTTGAGTCGCTGTGCTGTCCCTTATAAAGTGAAGGGACATACTTCCCTGTTCCTCAGTGCTG
AACTAATTCCTACAAACTTACTCCATACCACTCTTTTCTGCTTGCTTCTTTCCTAGCAAGGAAATTTTAACAGTTCTATTGGCTCAG
ATGGGTACATGCTGTCCTCCCTGACCTGTGGGATGGCCCCGGTGACTTCCATCACTGAAGGACTCCAGCAGGTAAGCCCTTTCTTAG
CCAGCAACCAGGTAGACATACAGACAGGTAGACTAACAAAACAAGAATCACTGTTACCAACTTCTGTGAGAAAAAGATGAAGACTAG
AGTGCAGTAGTTTTAAATTAAAGTTTGTTTCTCTCTCTGCCAACAGTAAGTAGGCAAGCAGGTTAAGGATATGTCCTGGGCAGAAGT
GAGGAACCAGGCTGCAAGTGGAGGAGGGGCCGTCGCTGTTGTTGGGTGCTCACCCATCTTTTCAGCAAAGAATCCTTGCTCTAAAGT
GTTCAAAAAGGAACACTTGGCCAAGAATTGTGGGGGGCAGCATTGTCCACAACCAGTGAAGTATTAGCTTAGAAACCATTTTCTAGC
TCATCAAACCTTCCAGTAGCTAAGCTGAAGGAGACTCAGAGAGCAGACCAGACACACCTTGCCTGTAAGAGCTTCATCCAGATTGCC
AGCACTGTCATAAAAAGCCAGTGTGGTCTCTCCTATCTGTAACCCAAGCCCTGTGTGGCAGATCCCCAGAGTTTGTGAGCCAGCAAC
CCATCCAGTCTGTGAGCCTAACAAAGATAGGTGCATGAGTTCTTTCCCCCATGTTTGTCTGTACACCACATGTGTGTAGTGCCCATA
AAGGGCAGAAGAAAACATTGGATTCCTGGAACTGGTGTTACAGAGAATAAGCTGCCAACTAGGTGCTAGGAATCGAATTCAAGCCCT
CTGGAAGAATGACGTAAGCTCTTGACCTCTGAACCATCTCTCAGGCCTGAAAGTTTACTCCTGTTGATAAGGTTCTGTGTTCCCCAG
ATGGTTAGGTGTAAGAGGGTTCACTAACCCTTAAGGAGTCTACTACCATGGAGTGCTAAGACTGGTGGGCAGTGCTAATGGCAGCCA
TGTCATTAGAGCTGTAATGGAAGGAACCTTTAGGGACAAAGCCAAGCATCATGGCTGAGAAAGCCCTGGTCTGTGTGAGGTGCATTC
AGATAACTCCTAGTTCTTTCTTGGGCCTTTTCCTAGCCTGTCTCATTCTGAAATGTCTCCCCATCATGATTTGCTTGACCTTGTTAC
AGGTAAGATCTTTGGGGACAGGGCCTAAGTCACTCTTACTTGTCACTGGACCCCAGCAGCCAGTACCTACATAACACAGGAGTTTTA
GATTAGTGCACAGAGTCTGCACCTGTCCACCTGTCTTGCCCGTTCACACAGTGATGCCAAATCTAAAACTCAGTCTAGGCTGGGTGC
TAGGCTGGATGCTTTTACCCAAGCCAGCATCGGGCTGTGATCCCCGACCACCCTGCTTCAGCTTCTTTCAGGCTCTGGTCCCACTCA
TCACCATGCTCTTCAGATCTCTCTCTCCCGAATTCTGCATCTCTGCTGATGATCCCAGAGTTGAGACCAACATCACAGTCCTTTACC
AGATCCACATATGTTGTGAGCTAGAACATGAAAAGTGACGTTGTGGTCCATCTGATAGAAGCCACTCTCCTTACACTGACTCTCCTC
TGCCACCCTCCCTAGCACTGAGATATGGGTATGAGCCAAACTCCAAGAGTATGTACATTTAAATAAAGTTGTTGCAAAATCAGATGT
TGCAAAAGAAAGAAATCCAAATTTGAGACATTTATTGGGACATTCAGAGGTCTACCTCAGCAGGCTGCTTTCTCGACAGGAAAAGAT
GGCAGTCACTCCCATAGAATAGAGCATAACCCCTTCCCAATCCCTGTGACCTAATCCCCCTATGCCAGCCACTGGCACAGATGCTGA
GAGACTTTCTTTGCTGTGTTTGTGAAATACAACTAACTCTACCTTACATAAATCCATCACTGGCTCCTTGGGGCTCAGCATGAAACT
TGAAATCCAGCGCACAGCACCAGCCACAGCCTGCTGCCACTTCTTTTTCCCTCTCCCTCGGCTCTCCTCATCTCCTCATGTATCTGG
AAATGTGTGTCTTTCCATGATGCCATTTGGAGTTGCTTTAATGAGAGACTGTCACTGGGAAGGGCACATGGCTATGGATAATTGCTG
CCTTGCCTCCTAGACTGGTTGGTCGCTTTCTTCTTTGATTATCTTTCCACTACAAGCCAATCGTATGCCCACCCGTAGTCGCAGCTC
CGTAGAAGGCTGAGGCCAGAGGATCACTGGTGACTAGGAGCCAAGACCAGTCTGAGCAACATAGTGAGAATTTGCCTCAATAAATAA
ACAGATCAGTTCAGGTAGGCGAGGAATGTATATTTCTCACGTCACGATCCTGAGCACCCAGCAAAAGGGCTTAATACTCAGAACAAA
AATTGTAGCCAAGGACACCTTGATTGTAGCATAAGTATCTGGAATGCGGAATTGTACCGTGCCTAGGAGTTATAACCCCGTCTTCTA
AGAATGCCTAGTTTGTTCTTTTTATGGTTGGAATTTGTTAATTCAGACAAATATTGTTGTCTGTGTGGCCGTGCTATCCACTGTACA
AAGCAGACGTATTCTGTTCTTGAGAATTACTGGCATTTACAAAGTTTTCATTATACCTGTGCTTGGAGCAGATCAGATGTGTCCTGG
AAACTGTTTAGAAGAGACTTAACTACTATAAAATGGGCTGTTGTCCTGTCCTGTTGGATGAGCTAACAGGTCTCCCTTTCTCTTGCA
GGTGGTCACCATGGGCCTTTTCCGAACAATTGCCTTGTTTTACCTTGGAAGTTTCGATAATATAGTTCGCCGCTGCATGGTGCAGAA
AGCAAAACCTGAAGTTGTAGACAAAACTGCCTAAACCTTGCCCCTTGGATGAAAGACTGAATCCAGTGATTTGAACAGTGTGCTGCT
AATGGAACACAAGTTTTGGCCTCCAGACTTTTGTATCTTGTTTTTGAATGTGTGAGATTGGACCCCGTGCTCTTCAGAAATCTGGCT
GTAAGCAGAGGGACATGAGGCCATCTCTACAACTGTTAAACACTATGCAAATATGGGCCAGGACACCTTTGATTTTCTGGGCTGTAG
GGGTGATAGTGTGAGAACTAATAACAGGAAAGCAGGGTAAAGAATAGCATTCCAGAACAGTAAATTCAGCTTTTCGGTCATTCTCCC
ATCCTACCCATAGAGATCAAGAAATGTCCTCTGTGGCGTTTCTGAGGTTTTGTTTTGTTTGTTTTTTGTTTTTGTTTTGTTTTGCTT
TTTTTTTTTTTTTTTGATTTTGGGTTTTAACTTTTTATGAAAGATGGTCCGGATTTTTATTAGTCTTTTTTTCTTTTTTGTAATTTT
TTTAGTGTATGTTATTCAAGGTGTGTCTTCCGAGTAGCCCGTGAGTCTGACTCTCAGCATGCCTTGCTGCGCCTGGGACTCGCTTCT
GCTAGTGAAGCTGGTTTCTCTCTCTTTGATCCCATAAAATTCGAGGGGGATGAGAGAGCAGCACAGAGGGCAAGGGGTGAGTCCTTT
GTGACGGCAAGCGGGGCTTTCTTGTCTTCTTAGACTGATGCTTACAACGTTTTCATTTTTATTCAAGGGGAAAGGCAGCCTCTTTAC
GTGTTTCGTGAAGAGAAATAAAATCTCCTAGCAGCTTAAGTTACAGTTTCTTCAGGAGCCATGATGACCTGAAGTTCACATTCCATT
TCAGCTCAGTTCCTAGTGCTTATCGCTCTTCCTAGTTTTGCTTATGCTACTGTAATATTTTTGTAGAAGAAAGGAAGGAAGAAAAAA
AAGATGGAGATCAGTGCAAATGTTTTTGACTTTTTTAATTAATCCATGAATTAATTAAAATAAAAAAATGAAAAGCATGACCTTCTT
ACTCTCAGAGCATACAATGGAGCTTCAGGGAAGGCAGCTGGTTGTGTACTTCTACTATTAAAAGAGGGTCAGGTTTGGAGGTTGGGT
GTTGAGCGGCTCTGTCTCGCGCACTGGCACCCAGCAGTATCTTCAGCATGGTAAGGAGCCCTCTGATGGCAGCCTGTGCGCGTGCAC
ATGCGCAAGACTGCGGTTCCATGGGCGAGCCTGCAACAAGTGCCTCAGATGAGTCCTGCCTGTATCCCACTCATCCTGTTTATTGTT
CCCCAGGCTCTAGGACAGTGTTCTATGTCCTGATATTTTACCCTGTTCTCCTTGTATACAAAATACAGCATTTCAGTTCACTCCACC
AGGAGAATACTACTTTGAAAGAAAAAACTTTATTGTTAAAAAACAGAAAACACCCGGGCATGGTGACGCACGCCTTTAATCCCAGCA
CTCAGGAGGCAGAGGCAGGCCGACTTTTGAGTTCGAGGCCAGCCTGGTCTTCAAAGTGAGTTCCAGGACAGCCAGGGCTACACAGAG
AAACCCTGTATAGGAAAAAAACCAAAACC
MOUSE mRNA SEQUENCE:mR22-022.1 (Seq ID No: 2)
TCGCTCGCAAACCCAAACACTCCCGGCTGCTGGTGCATGTGATCTCCCAGTAGTCGCTCGGCAGAGATGTTGCTGTTGGCCGCTGCC
GGCCTCGTGGCCTTCGTGCTGCTCCTCTACATGGTGTCGCCGCTCATCAGTCCCAAGCCCCTCGCGCTGCCCGGCGCGCACGTAGTG
GACAAGCTACTGCAGGCGAAGAAAGACATTGAAAAGCACTCTATTAATGACAAACAGGTGGTGCTGTGTATCTCAGTTGATGTGTCT
CAAGACTATAACCAAGTGGAGAATGTCATAAAGCAGGCACAAGAGAAGCTGGGTCCTGTGGACATGCTGGTCAACTGTGCAGGCACC
TCTATGTCAGGAAAGTTTGAAGAGCTTGAAGTTAGTAGTTTTGAGAAATTAATGAGCATAAATTACCTGGGCAGCGTGTACCCCAGC
AGGGCAGTAATCACTACCATGAAGGAGCGACGGGTGGGCAGGATCGTGTTTGTGTCCTCTCAGGCAGGACAGCTGGGACTGTTTGGT
TTCACGGCCTACTCTTCATCCAAGTTTGCCATAAGAGGATTGGCAGAAGCTCTGCAGATGGAGGTGAAGCCGTACAATGTGTACGTC
ACTGTGGCCTACCCACCAGACACCGACACGCCGGGGCTGGCTGAGGAAAACAAAACGAAGCCCCTGGAGACCCGGCTTATCTCAGAG
ACCACAGCTATTTGCAAACCAGAGCAGGTGGCCAAACAAATTGTCAAAGATGCCATACAAGGAAATTTTAACAGTTCTATTGGCTCA
GATGGGTACATGCTGTCCTCCCTGACCTGTGGGATGGCCCCGGTGACTTCCATCACTGAAGGACTCCAGCAGGTGGTCACCATGGGC
CTTTTCCGAACAATTGCCTTGTTTTACCTTGGAAGTTTCGATAATATAGTTCGCCGCTGCATGGTGCAGAAAGCAAAACCTGAAGTT
GTAGACAAAACTGCCTAAACCTTGCCCCTTGGATGAAAGACTGAATCCAGTGATTTGAACAGTGTGCTGCTAATGGAACACAAGTTT
TGGCCTCCAGACTTTTGTATCTTGTTTTTGAATGTGTGAGATTGGACCCCGTGCTCTTCAGAAATCTGGCTGTAAGCAGAGGGACAT
GAGGCCATCTCTACAACTGTTAAACACTATGCAAATATGGGCCAGGACACCTTTGATTTTCTGGGCTGTAGGGGTGATAGTGTGAGA
ACTAATAACAGGAAAGCAGGGTAAAGAATAGCATTCCAGAACAGTAAATTCAGCTTTTCGGTCATTCTCCCATCCTACCCATAGAGA
TCAAGAAATGTCCTCTGTGGCGTTTCTGAGGTTTTGTTTTGTTTGTTTTTTGTTTTTGTTTTGTTTTGCTTTTTTTTTTTTTTTTTG
ATTTTGGGTTTTAACTTTTTATGAAAGATGGTCCGGATTTTTATTAGTCTTTTTTTCTTTTTTGTAATTTTTTTAGTGTATGTTATT
CAAGGTGTGTCTTCCGAGTAGCCCGTGAGTCTGACTCTCAGCATGCCTTGCTGCGCCTGGGACTCGCTTCTGCTAGTGAAGCTGGTT
TCTCTCTCTTTGATCCCATAAAATTCGAGGGGGATGAGAGAGCAGCACAGAGGGCAAGGGGTGAGTCCTTTGTGACGGCAAGCGGGG
CTTTCTTGTCTTCTTAGACTGATGCTTACAACGTTTTCATTTTTATTCAAGGGGAAAGGCAGCCTCTTTACGTGTTTCGTGAAGAGA
AATAAAATCTCCTAGCAGCTTAAGTTACAGTTTCTTCAGGAGCCATGATGACCTGAAGTTCACATTCCATTTCAGCTCAGTTCCTAG
TGCTTATCGCTCTTCCTAGTTTTGCTTATGCTACTGTAATATTTTTGTAGAAGAAAGGAAGGAAGAAAAAAAAGATGGAGATCAGTG
CAAATGTTTTTGACTTTTTTAATTAATCCATGAATTAATTAAAAT
MOUSE PROTEIN SEQUENCE:mP22-022.1 (Seq ID No: 3)
MLLLAAAGLVAFVLLLYMVSPLISPKPLALPGAHVVDKLLQAKKDIEKHSINDKQVVLCISVDVSQDYNQVENVIKQAQEKLGPVDM
LVNCAGTSMSGKFEELEVSSFEKLMSINYLGSVYPSRAVITTMKERRVGRIVFVSSQAGQLGLFGFTAYSSSKFAIRGLAEALQMEV
KPYNVYVTVAYPPDTDTPGLAEENKTKPLETRLISETTAICKPEQVAKQIVKDAIQGNFNSSIGSDGYMLSSLTCGMAPVTSITEGL
QQVVTMGLFRTIALFYLGSFDNIVRRCMVQKAKPEVVDKTA*
MOUSE PANTHER CLASSIFICATIONS
              FAMILY (SUBFAMILY)
                            SHORT-CHAIN DEHYDROGENASE-RELATED (FOLLICULAR VARIANT TRANSLOCATION
PROTEIN 1)
              BIOLOGICAL PROCESS
                            Biological process unclassified (2.99.00.00.00)
              MOLECULAR FUNCTIONS
                            Oxidoreductase (1.19.00.00.00) > Reductase (1.19.06.00.00)
                            Oxidoreductase (1.19.00.00.00) > Dehydrogenase (1.19.05.00.00)
MOUSE GENE ONTOLOGY
                            No Gene Ontology
MOUSE PROTEIN DOMAINS (INTERPRO SIGNATURES)
                            IPR002198 (SDRFAMILY)
                            IPR002198 (adh short)
MOUSE mRNA SEQUENCE:mR22-022.2 (Seq ID No: 4)
CGCCCTCGCTCGCAAACCCAAACACTCCCGGCTGCTGGTGCATGTGATCTCCCAGTAGTCGCTCGGCAGAGATGTTGCTGTTGGCCG
CTGCCGGCCTCGTGGCCTTCGTGCTGCTCCTCTACATGGTGTCGCCGCTCATCAGTCCCAAGCCCCTCGCGCTGCCCGGCGCGCACG
TAGTGGTCACAGGAGGCTCCAGTGGCATTGGGAAGTGCATTGCTATTGAGTGCTACAAACAAGGAGCATTTATAACTCTGGTTGCAC
GAAATGAGGACAAGCTACTGCAGGCGAAGAAAGACATTGAAAAGCACTCTATTAATGACAAACAGGTGGTGCTGTGTATCTCAGTTG
ATGTGTCTCAAGACTATAACCAAGTGGAGAATGTCATAAAGCAGGCACAAGAGAAGCTGGGTCCTGTGGACATGCTGGTCAACTGTG
CAGGCACCTCTATGTCAGGAAAGTTTGAAGAGCTTGAAGTTAGTAGTTTTGAGAAATTAATGAGCATAAATTACCTGGGCAGCGTGT
ACCCCAGCAGGGCAGTAATCACTACCATGAAGGAGCGACGGGTGGGCAGGATCGTGTTTGTGTCCTCTCAGGCAGGACAGCTGGGAC
TGTTTGGTTTCACGGCCTACTCTTCATCCAAGTTTGCCATAAGAGGATTGGCAGAAGCTCTGCAGATGGAGGTGAAGCCGTACAATG
TGTACGTCACTGTGGCCTACCCACCAGACACCGACACGCCGGGGCTGGCTGAGGAAAACAAAACGAAGCCCCTGGAGACCCGGCTTA
TCTCAGAGACCACAGCTATTTGCAAACCAGAGCAGGTGGCCAAACAAATTGTCAAAGATGCCATACAAGGAAATTTTAACAGTTCTA
TTGGCTCAGATGGGTACATGCTGTCCTCCCTGACCTGTGGGATGGCCCCGGTGACTTCCATCACTGAAGGACTCCAGCAGGTGGTCA
CCATGGGCCTTTTCCGAACAATTGCCTTGTTTTACCTTGGAAGTTTCGATAATATAGTTCGCCGCTGCATGGTGCAGAAAGCAAAAC
CTGAAGTTGTAGACAAAACTGCCTAAACCTTGCCCCTTGGATGAAAGACTGAATCCAGTGATTTGAACAGTGTGCTGCTAATGGAAC
ACAAGTTTTGGCCTCCAGACTTTTGTATCTTGTTTTTGAATGTGTGAGATTGGACCCCGTGCTCTTCAGAAATCTGGCTGTAAGCAG
AGGGACATGAGGCCATCTCTACAACTGTTAAACACTATGCAAATATGGGCCAGGACACCTTTGATTTTCTGGGCTGTAGGGGTGATA
GTGTGAGAACTAATAACAGGAAAGCAGGGTAAAGAATAGCATTCCAGAACAGTAAATTCAGCTTTTCGGTCATTCTCCCATCCTACC
CATAGAGATCAAGAAATGTCCTCTGTGGCGTTTCTGAGGTTTTGTTTTGTTTGTTTTTTGTTTTTGTTTTGTTTTGCTTTTTTTTTT
TTTTTTTGATTTTGGGTTTTAACTTTTTATGAAAGATGGTCCGGATTTTTATTAGTCTTTTTTTCTTTTTTGTAATTTTTTTAGTGT
ATGTTATTCAAGGTGTGTCTTCCGAGTAGCCCGTGAGTCTGACTCTCAGCATGCCTTGCTGCGCCTGGGACTCGCTTCTGCTAGTGA
AGCTGGTTTCTCTCTCTTTGATCCCATAAAATTCGAGGGGGATGAGAGAGCAGCACAGAGGGCAAGGGGTGAGTCCTTTGTGACGGC
AAGCGGGGCTTTCTTGTCTTCTTAGACTGATGCTTACAACGTTTTCATTTTTATTCAAGGGGAAAGGCAGCCTCTTTACGTGTTTCG
TGAAGAGAAATAAAATCTCCTAGCAGCTTAAGTTACAGTTTCTTCAGGAGCCATGATGACCTGAAGTTCACATTCCATTTCAGCTCA
GTTCCTAGTGCTTATCGCTCTTCCTAGTTTTGCTTATGCTACTGTAATATTTTTGTAGAAGAAAGGAAGGAAGAAAAAAAAGATGGA
GATCAGTGCAAATGTTTTTGACTTTTTTAATTAATCCATGAATTAATTAAAATAAAAAAATGAAAAGCATGACCTTCTTACTCTCAG
AGCATACAATGGAGCTTCAGGGAAGGCAGCTGGTTGTGTACTTCTACTATTAAAAGAGGGTCAGGTTTGGAGGTTGGGTGTTGAGCG
GCTCTGTCTCGCGCACTGGCACCCAGCAGTATCTTCAGCATGGTAAGGAGCCCTCTGATGGCAGCCTGTGCGCGTGCACATGCGCAA
GACTGCGGTTCCATGGGCGAGCCTGCAACAAGTGCCTCAGATGAGTCCTGCCTGTATCCCACTCATCCTGTTTATTGTTCCCCAGGC
TCTAGGACAGTGTTCTATGTCCTGATATTTTACCCTGTTCTCCTTGTATACAAAATACAGCATTTCAGTTCACTCCACCAGGAGAAT
ACTACTTTGAAAGAAAAAACTTTATTGTTAAAAAACAGAAAACAGCCGGGCATGGTGACGCACGCCTTTAATCCCAGCACTCAGGAG
GCAGAGGCAGGCGGACTTTTGAGTTCGAGGCCAGCCTGGTCTTCAAAGTGAGTTCCAGGACAGCCAGGGCTACACAGAGAAACCCTG
TATAGGAAAAAAACCAAAACC
MOUSE PROTEIN SEQUENCE:mP22-022.2 (Seq ID No: 5)
MLLLAAAGLVAFVLLLYMVSPLISPKPLALPGAHVVVTGGSSGIGKCIAIECYKQGAFITLVARNEDKLLQAKKDIEKHSINDKQVV
LCISVDVSQDYNQVENVIKQAQEKLGPVDMLVNCAGTSMSGKFEELEVSSFEKLMSINYLGSVYPSRAVITTMKERRVGRIVFVSSQ
AGQLGLFGFTAYSSSKFAIRGLAEALQMEVKPYNVYVTVAYPPDTDTPGLAEENKTKPLETRLISETTAICKPEQVAKQIVKDAIQG
NFNSSIGSDGYMLSSLTCGMAPVTSITEGLQQVVTMGLFRTIALFYLGSFDNIVRRCMVQKAKPEVVDKTA*
MOUSE PANTHER CLASSIFICATIONS
              FAMILY (SUBFAMILY)
                            SHORT-CHAIN DEHYDROGENASE-RELATED (FOLLICULAR VARIANT TRANSLOCATION
PROTEIN 1)
              BIOLOGICAL PROCESS
                            Biological process unclassified (2.99.00.00.00)
              MOLECULAR FUNCTIONS
                            Oxidoreductase (1.19.00.00.00) > Reductase (1.19.06.00.00)
                            Oxidoreductase (1.19.00.00.00) > Dehydrogenase (1.19.05.00.00)
MOUSE GENE ONTOLOGY
              No Gene Ontology
MOUSE PROTEIN DOMAINS (INTERPRO SIGNATURES)
                            IPR002198 (SDRFAMILY)
                            IPR002347 (GDHRDH)
                            IPR002198 (adh short)
HUMAN GENOMIC SEQUENCE:hD22-022 (Seq ID No: 6)
GGCTCCTCCTCTTCCCCTCCGCCGCGGCCCGCCCCGGCCCGCAAACCCAAACACTCCAGGCGCCCGCCCGCCGCGCGTGATTCTCGC
CTCGCCGCAGCCCAGCCCTGCGCGCCTTGCCCGGCGGCCCCCGCCCGGCCGCTCCGGGCCCCTGGCCCCGCGGAGCGATGCTGCTGC
TGGCTGCCGCCTTCCTCGTGGCCTTCGTGCTGCTGCTGTACATGGTGTCTCCGCTCATCAGCCCCAAGCCCCTCGCCCTGCCCGGGG
CGCATGTGGTGGTGAGTGGCCTCCTGTTGCTGCCCCCCGACTTACCGGCCCGCGCGGCCTTTCCGCCGCCGAGGCCGCGTAGCCTAC
GTCCCTTTTTCCAGGCCGCGGTCCCCAGGAAGGCATGGCCAAATGGGGAAGGTCACCTGCCGCCGTCCACCCCGGCGCTTTTCCCCC
GGCCCCGGCACCCGGCCTCGCGGACCCGCCCTCCGCAGCCTCGGGCTCCTCCTAGGGCCGGAGAGCGTGCGACGGGGGACCCCGCCG
GCCGCAGGGTTCCCGGGGCCCCAGGGGTTCTGTGGCTGCTCGTGAATCCATGCAACATCCCTGGGATGTGGATGTTTTGTCCTCAGT
TTTTCCGATGAGTCAACTGAGGCACAGAGAGGCTGAGAAACTTTGCCAAGGACACAGCGGTCGGTGGTGTGCAGGGTGCGGCGCGCG
GCCCCGGGCGGGGGCAGACAGCGGGGAAGTGGGGTCCCAGCCGCCGGGACCCGCCGAGGCAGCTCGGAACTGGAAGATGGCCTGTGC
GAGTGACGGGAAGAGCCGCTGCCAGAGGATTCCAGGAAGTGACTCACCTGGGGAAACTGCTCAGAACCCAGCGGGCTGGCGGTGGTG
GCTTAGCGGTATTGGGACGCCTGTCCCGTCTACACTGCTTGTCTCTGAGCTCAGCGTGGCATCTGCTGATTGCTGCTCCAATGCAAT
CCTTTGCTGTTTTCTAGTGGGAAGGATGAGCTAAACGACAAGGCTTGTGTGCGAGGAATGAAGGAGAGGTGGATAGGTGTGATTTAT
TTCTTGGCGGAAGCTCGCATATTCAGCGATCCCTGTTCTGCAAATGTGGCACCTTTCTTAAACAGAGAGAATGCCTCCTTGCCTGCG
CCCAGCACTTTCCCGTTTGCAGTGGCTTTCTCACGAGCATTCTCGTTTGGATCTCCCAAACCCGAGGCACAGTGAGGCGAGGAGTTA
GGAGTAAGGAGGGTTGGTTACAAAATTGGCCGCACACCTCTTCCTGTCCTATCTCTGCACCGGAGATCCGAGCCTCCCCGCTGAGCT
GGTATAAAATAAGATGGTCGTTTTTCTTAGTATGCTTTAAATCTGTACAAGGGTTTGTGAATGAGAAAATGTATCTGTTTAATTTCT
CCTTTGTAAAGCTATTAGCTTTTTCCAACACCTCCTGTTCCTAATGGGGAAAGAGTGTGGATTCATTACAAAAAATAAATAAGAGAA
AGGCAAAACTTGACACTCTAAATTCTGAAAGTTTTACATCCGCAAGAGAACTGCTGCCTCAAAAGCGTGGCTTTAAACCTTGGATCC
TTTCAGAGCTTGAGTTTATAGAGCATAACTTTGCAAAAACTAAAGCTAAACGCAGAATGAAAATTGGAATTCCTTATTTGTAAGACA
GTTTCACTGAGCCTTGACTCCAGTCCTTCCAGCCCCATCCCGCGGCAGGTCATAGGTGAGATAATAAATGCTGTGTCTGTTCAGGTG
GACCAAGAAGAATTTATTTTTACCATCCCGCTTTAATACGCAGGATTATTATTTCACACTCTAAGTAATGTGAGAATGACATGAATA
GTTCACATGGTGTCACCATCTGTCTTCTTTTTTAATGTCATAAGAAGAGTGAAAAGTGTAATTGTTCTTGAGATTTGTTAGGAGTTG
ACTGTGGTATTGTTGGGAGAGGGAGCTGATGGTTAAACTGTCTGCCCATACATAATTAAGAACAAACTTATAACAATTGTTTCATTT
TTAAAACAATTTTATATACTAGTGTTTTCTATCTAGTTACGGTAGTAAAATTGCAGGAAAAAAGCAGAAAATCACCCACCTCAGCCT
CCGAAAGTGCTGGGATTACAGGCGCGAGCCATCGCGCCCGGCCAACAACAGCTATATTTTTACAGTAATCTGCTGATAACTGGCTGA
TTACCGACCAAATAAATCTCTTCAGTGGTGGCAGGTTGAAGCTAAAAAGAAGTAAGGGATGCCTATGAGTCCCATAGAGGTAGGTGC
ATTTGCGTAGGAGACACTTCCCAATGATGTTAAAATCCTAACATGGAGTCAGAGATCTTCCTCCCAGCATGATTTATGTATAACAGT
GAATATTAGGAAAAATCTAAATGTCTGGTTATAGGGAAACGTTGAAGTTTTGATGCAGCTATTTGATGAAGTATTGTGAAACCTTTT
CGTTTTTTGACAAATAAGACATTACAATATAATTTTTTAAAAACAGGAGACAATACTATGCAATATTATGTAATTTGCATAGGGACA
GTGACTAGAACGATATGGCCGGGTACAGTGGCTCACTCCTATAATCCCAGCACTTTGGGAGGTCAAGGAGAGTGCATAACTTGAGGT
CAGGAGTTCGAGACCAGCCTGGCCAACATGGTGAAACCCCATCTCTACTAAAAATACAAAAATTAGTTGGGCGTGCTGGCGCACGCC
TGTAATCCCCACTACTTGGGTGTCTGAGACAAGAGAATCGCTTGAACCTGGGAGGCGGAGGTTGCAGTGAGCTGAGATCGTGCCACT
GCACTCCAGCCTGGGTGACTGAGCAAGACTGTCTCAAAAAAAAAAAAAGAAAAATAATGTTGCATTGTTCACTTATTCAGTGAAATA
TATAATATATATTGAGCCCTCCCCAGGCTAAGTGCTAGGAATATAGTGGAGAACAGAAAAATAATTGGTATTTTTAATGGAGTTTAT
GGGTAGGTGATAGGAGAGGTGGTTGTGAATCAGATAATCACACAAATAAATCTAGAGCTGTTTATTATGTGCATGTCATAAAGAAGA
AATACAGTGCTGAAAAGTGTGTCAGTGTGGGGGCAGGGGAGACAGTGAAGGCTTTCCTGAGGAAGTGGTATTTGAGCTGAGATTTGA
TGGGCGAGTAGGTGTCAGATGAAGTGGGGAGAAGAGCATTCGTTGCAAGGGAAGTAGGATGTGCCAAGGTACTGTGGTTGGAGGGTT
TTTACAGTAATCTTCTGCTGATAACTGGCTGATTACCCACCAAATAAATCTCTTCAGTGGTGGCACGTTGAAGCTAAAAAGAAGTAA
GGGATGCCTATGAGTCCCATAGAGATAGGTGCATTTGCGTAGGAGACACTTCCCAATCATGTTAAAATCCTAACATGGAGTCAGAGA
TCTTCCTCCCAGCATGATTTATGTATAACAGTGAATATTAGGAATAATTCCATAATTGAAAGCCAGTATAGCTGAACTGTAGATAGG
GGTAGAATAGTGCAAGAAGAGCTTTACTTTTCAGGATTTCCTTGTGATTGTATATTACTTTTATACTAAGCAGTCATTTCAAATAAA
TATTCAGGTGGCCATTCTTTTCATCTGTTGTAGAGCTAATCTTGCATAAATGTGCATTAAGAAAATAAGATTCAACCAACCCAAATG
TCCAACAATGATAGACTGGATTAAGAAAATGTGGCACATATACACCATGGAATACTATGCAGCCATAAAAAATGATGAGTTCATATC
CTTTGTAGGGACATGGATGAAATTGGAAACCATCATTCTCAGTAAACTATCGCAAGAACAAAAAACCAAACACCGCATATTCTCACT
CATAGGTGGGAATTGAACAATGAGATCACATGGACACAGGAAGGGGAATATCACACTCTGGGGACTGTGGTGGGGTCGGGGGAGGGG
GGAGGGATAGCATTGGGAGATATACCTAATGCTAGATGACACATTAGTGGGTGCAGTGCACCAGCATGGCACATGTATACATATGTA
ACTAACCTGCACAATGTGCACATGTACCCTAAAACTTAGAGTATAATAAAAAAAAAAAAAAGAAAAAAAAAAAGAAAATAAGATTCA
TTTGGATTTGGAATCAGAGGCAGACTAATGTATTATTTATGAGCTAGACTTATAGGGCACTTTTCCACCAGTGAACTCAGAAAAATA
TTATAGGTGTGCAATTTATGGTTCCATAAGAAAACAGTCAAAGCAGCAGGCCTTGCTGATAGTATCTTTCATGCTTACACTTACTCC
AGTCATTAAGTTTGGGATGAAGAAGTTAACAACAGCTAATCATTTAGTTCTTGTAGATTCATTTTAAGAGCCTGTCATAGTTGTTTT
TTATAAAAACTTCCTACAGAGGGGAGATTTCAAGTGCTAAGAATATTTTATGATTTTGTTTTAGGTTACAGGAGGTTCCAGTGGCAT
CGGGAAGTGCATTGCTATCGAGTGCTATAAACAAGGAGCTTTTATAACTCTGGTTGCACGAAATGAGGTAAGGCTTCTAGGTTCATT
ATTACTGACTTGCTTCTTCGACTTGAAATTAGTCAAAGAGGCTGTGCTAAACATCTTAGCATTCACACCTAGAAAGCGCTTCTTCCT
TGATGTGAGATGTGTTTCCTGAACTACTGATTCTCTCTAGTATTTAGGCCAAACACTTCTCTGTTGAAATCAGGTCGGCCCCACAAG
TATTTATTTAGTACCATTGGCATATTAGGCAACATCATCATAGATACTGCAGATCCTGAGATGGGAGAGGCCAAGGTCTAGTTGAAG
GAGCACACCTGGCCATTGGAAGCCAGGGAGGGCCCTTGGCCAGGTCAGAAGTGCAGGAAGCAGTGATTCTGGAGCTGATACCTGAAG
GTGTGTAGGTGTTTCTCTATAATTTTTCACTACGATTTGCTTAGGACATGATATATATCTCTAGTGAAATCTATACACCATAGAGGA
ACCTCGTCCCTCCTTAGAAGTTTAGCAAAATGTGTTTTTCTGTATTAAGTCAACACAGAATCTGTTTTTATTTTTTGTCAGGGTTTG
TTTTTGTCTTGCCAAAGTTGTTTTTAGAGCACAAAGCAGTGACACTGTGCACGTGAACTGTCATGACGTCCTCTTGCTCCCCATATG
GGGACTGGAGGAAAGTGACCACACAGCTGGCTCGTCCGCTGCAGCAATTCTAGTGCTTGACACGTAGTACATGCTCAGACATGTTTG
TTGAATGAAGAAATGAAGGGAGGAATGATGGAGTGCCTGGGAATGTCAGCATGGCATACTTCCATGGGGATCTTGGATTCACCCATG
TGCAAGGAGGGTGATAGTATGGTAACTCCTCAAATGGGTGGAGGGCTGGACAGGACCCATTAGGAATGGTAGGCCAGGAAGGGCCTT
GGGGGAAAATTTAAATAATGTCGCAGACTTAGTCTTGGACCTCTATAATTTCTTTTATTTTTTTCTTTTTTCTTGAGACAGCATCTC
ACTCTGTTGCCCAGGCTGGAGTGCAGTGGCATGATCTCGGCTCACTGCAACCTCTGCCTCCTGGGTTCAAGTGATTCTCCTGCCTTA
GCTTCCTGAGTAGCTGGGACTACAGGCGCCCACCACCACGCCTGGCTAATTTTTGTATTTTTAGTAGAGACAGGGTTTCACCATGTT
GGTCAGGCTGGTCTGGAACTCCTGACCTTGTGATCCGCCCGCCTTGGCCTCCCAAAGTGCTGGGATTACAGGTGTGAGCCACCACAC
CTGGCCTGGACGTCTATAACTTCTTTTGCAAACCAAGAAAGACAAAATTAGCTACCTTTTATTTATAGCTTGACTTTTCTAACAATG
AATTTGCTTTGGCATTGAAGCCACCTTACATAAATGAAAACACAAATTTGATGAAATGGCATTTTGTGAGTTTTAAACACCAGTTTT
GATGCAAGTAAATAAAAGTGGAGGCAGAGAAGGCCAGCATTTCAGGCCCCCAGGAGCTCATATGGGGCCCTTGCATTGCTAGAAATG
AATGAACTGAATGAAGAACCTGTGGTGTTGAGTAATTCATTCTTTTTTTTTTTTTTTTTTTTTTTTTTTTGAGACGGAGTTTTGCTC
TGTAACCCAGGCTGGTGTGCAGTGGCGCTGTCTTGGCTCACTGCAACCTCCACCTCCGGAATACCTGGGATTACAGGTGTGTGTAAC
CACACCTGGCTGATTTGTGTGTGTGTGTGTGTGTATATATATTTATATATATATAAAATATATATTTTTATTATATATATTATATAT
TATATATATAAAAATATATATTTTTATTATATATATTATATATTATATATATATAAATATATATTTTTATTATATATATTATATATA
TATAAAATATATATTTTTATTATATATATTATATATTATATATATATTATATATATATATATTTTTTTTTTAGTAGAGACAGGGTTT
TACTATGTTGGCCAGGCTGGTCTCAAACTCCTGACCTTAAGTGATCCACCCACCTCGGCCTCCCAAAGTGCTGGGCTTACAGGCGTG
AGCCACTGCGCCCGGCCTCACTGTGTCACCCAGGCTGGAGTGCAGTGACACAATCTCGGCTCACTGCAACCTCCGCCTCCCAGGCTC
AACCAATCCTCTCACTTCAGCCTCTTGAGTATCTGGGACCACAGGTGCTCGCCACCATGCTTGGCTAAGTTTTTGTATTTTTGGTAG
AGACGGGGCTTCACCATGTTGCCCAGGCTGGTCTCAAACTCCTGAGCTTGGGTGATCCACTCACCTTGGCCTCCCAAAGTGCTGGGA
TTACAGGGGTGAGCCACCATGCCTGGCTGAGAAATTCTTTCTTTTCTAATGTAATTTTTAATTCCAGTCTTTGCAAAGTCAAGCTGT
ATGTATCTTTAGTCATAGGACAATATTATTTCATTATAGACATATTCAGGTTTAGAAACTCTGCATGCCTTTTGGAAGAATTCGCCT
CTCCTTTGGTTCCTCTCTATTGCATGATATTATTTAAGTTACTTTTTACAGCAAATTAAGATAATCTGATATCTGTTTTCTTGAGAC
TAAGTAAAACAAACAAAATTAGGACCTTGGAAGAATTACATAATTACACAGGTAACCTTGCTTAAGAGTATTTTATAGTAGCCAGGC
CTAGTTTACCCAGTCTATCCAGAAAGTGATTCTTACTATGTTTGGGTTTATAGTAGAAGAAGAAAATATTGGACAAAAAGCAGTATT
GACAGGGAGTCACACATACCCTCAACTTTGGGGAGCAGTACATATTAATAGAGTTGTTCTTGTCTTATATCACAACTAAATAATAAG
ACATTTATTAGAACTGTAGAGTCAGCCTCAGCAATTAGTACTGTTAACTGGGATTTTGAGGTTTATCTGTTTTCTACTATCTTTCTC
TAATTAATTTTCAAACTTTTATCACTTATTCATAGCTTAACATACTTCACAACTTCTTTCTTTCATAGGCTGTGGCCCAGTCTGTCT
ATGCAATGTAGATACTGTTTTGTGTGTTTTCTGGAATTTAATACATACATATAGAAATAATTGCTTTTTTTCTCTCCTTTGCAATAA
ACGGACATGCATATAGACGGTCATCACGACTAATTATTCTGTATTGCTTTCAGGATAAGCTGCTGCAGGCAAAGAAAGAAATTGAAA
TGCACTCTATTAATGACAAACAGGTAAATCTCTGTCTTCAGAATGCATTTTCTGTATAACTCAGCACAGCTGTATAAAGGAAAAACA
GAGTTACTTCACCTGTGAATAGTTAATTGAAAGCATATATGTACTGTTTTTTAAAAGGTGGAATTGTTTGAATGGTCTAGTAACTCA
GAATATCACACACACGCAAAATGAATAGACTCCCACACACATTCATATCTACAAATGGTCAAATAACAGAATGTTTTACTGGCAAAT
AATTTCATTTTAATTTTTTTTTTAATGAACTTTATACAAATGCTTAAGAGGAAAGGAATATTTAAAATTTATTTGGTTATGGAATTT
TTGATGAATAAAATGCTATTTATATTTCAGTATAAAACATAATAGCAAATTAATAATCACACACCCATCACCCAGCCTAAGAAGTAG
AAATGATCAGGGTCTCTGAAGCCCCCACAGCAGCCTTGTCCCTCTTTTCTTGTTCCTAGAGGGGCAAATGCCAACCCGATTTTTGGT
GTTAATCCTTTCTTTTTCTTTTCTTTTTTCTTTCCTTTTTTTTTTTTTTTTTTTTTTTGAGGCAGTGTCTCAATCTGTCATCCAGGC
TGGAGTGCAGGGGTGCGATCTCAGCTCACTGCAGCCTCGACCTCCTGGGCTCAAGCAGTCCTCCCACCTCAGCCTCCCTAGTAGCTG
AGATTATAGACACGTGCCATCATGCATGGCTAATTTTTGTATTTTTGGTAGAGATGGGGTTTCGCCATGTTGCCCAGGCTGGTCTTC
AACTCCTGGGCTCAAGCAGTCCGCCTACCTCTGCCTCCCAAAGTGCTGGTATTATAGGAGTGAGCCACTGTGCCCTGCCTCTTTTTC
TTTTTAATTTTACCATATATGCACATAACTCTAAATAATAATATATTGTTTAGTTCTTTTTTTTTTTTTTTTTTTTTTGAGACAGAG
TCTCTCTCTGTCACCCAGGCTGGAGTGCAGTGGTGCCATCTTGGCTCACTGCAACCTCCACCTCTCGGGTTCAAGCGATTCTCCTGC
CTCAGCCTCCTGAGTAGCTGGGATTACAGGTGTGTGCCACCACACCCGGCTAATTTTTTTTGTATTTTTAGTAGAGACAGGGTTTTA
CCATGTTTGTCAGGCTAGTCTCAAACTCCTGACCTCAGGTGATCTGCCTGCCTTGGCCTCCCAAAGTCCTGTGATTATGGGCGTGAG
CCACCATGCCTGGCCAATATACTTAGTTCTACTTCTTTTATTATATCATTCTTATTTTTTAACAGCTTTATGAGATATAATTTATAT
ACCATACAGTTGACCCATTTAAAATATATAATTCAATGATTTTTAATGTATTCATAGAGTTGTGCAACTGTCATCAAAATCAATTTT
AGAATGTTTTCCTCACCCCGTGAAGAATCTCCATGTGTATTAGCAGTCACTTGCCATTTCCTCCCAGCCTCTTCTCAGCTAGGCAGT
CACTCATCTACTTTCTTTCTCTATAGATTTGTATAACTTCCTTCTTTATGCTTCTTATGCAACTTGATTTCTTCACTCAATATTATT
ATTATTTATTTCTTATTTATTTATTTATTTTTGAGATGGAGTCTCGCTCTGTTGCCCAGTCTGGAGTGCAGTGGCACAATCTCAGCT
CACTGCAACCTCTGCCTCCTGGGTTCAAGCCTCAGCTTCCTGGGTAGTTGGGATTACAGGCGCGCGTCACTACGCCCAGCTAATTTT
TGTATTTTTAGTAGAGACAGGGTTTCACCATGTTGATCAGGCTGGTCTCGAACTCCCAACCTCGTGATCCGCCCGCCTCAGCCTCTG
AAAGTGCTGGGATTACAGGTGTGAGCCACCATGCCCAGACCACTCAATATTATTCTTTAGGCCAGGTGCGGTGGCTCATGCCTGTAA
TCCCAACATTTTGAGAGGCCGAGACGGGCAAATCAGTTGAAGGTAGGTGTTTGAGATCAGCCTGGACAAAGTCGTGAAACCCTGTCT
CTACTAAAAATATAAAAAGTTAGCTGCACCTGTAACCCCAGCTACTCAGGAGGGAGGCTGAGGCAGGAGAATCACTTGGATCTGGGA
GGCAAAGCTTGAGGTGAGCAGAGATTGCACCGCTGTACTCCAGCCTGGGTAACAGAGGCAGACTCCATCTCAAAAAAAAAAATATAT
ATATATATATATATATGTATGTATGTATGTATGTATATATATGTGTATATATATATATAATTTTAAAAATTCATTTATGTTGCTGCA
AAAAGTTGGAGTAAATTTTTTTTTTTTTACTGCTGCGTAGTATTCTATTGTATGTATAGACTACATTTTGTTCATCAGTTCAGCAAT
TGGTGGATATTTAGTTTGTTCCCAGTATTTTTGTTAGTTTGTTTTGTTTTTTACTATTAGAAAAGCTTTCAGTATTCTCTTTGTGCA
CGTGTGCAAGAATGGTACATACCCAGAAATGGAATTGCAGAGCTGTAGTGTATGCAGTTCTTCAACCTCACCAAAGTGGTTAAGCTC
CCATAAGTAGGAATGCGGTTTCTGTTGCGCCACATCCCTGCCAATACTTGGTATTGACAGACTTTTTACTTTTTGCCTTTCTGATGA
GTTTTGAATAGTATTTCACTGTGGTATTCATTGACTTTTTCCCTTCTAGAATGTTAGAGATCTTTCCATGTGCATTAAGTAAATTTG
CCTTAAATTGCTGCAGCCTAGGATTGAAGTACACCAGAGCTTATTCCGTCAGGTCCCTGCTATTGAGCTGTTTCCATTCTTTTTATT
TTATTTTGTATTTTTATTTTTGCAATTGCAGTCTGTGCTGCAAGTAAAAATCCTTGTGTATATCTTCTGCACATGTGAGTTTTTCTC
TAAGACTGTGGTCTTTACCCCCTTCTCTGAAGCCCACAAGCATTGGAAAAACACCAGCTCTGTAAATTGCCCCGTTTTTTCCCGTAT
TCCTGTAATATCTGATAGACGAAGGAGTATGGTGTGAAGTCATGGAGTCAGTGTTCAGGGCCAAGGCCAGTCTTGCCTGCTGCCAAC
ACTGTCTCCGCTCTGGGCTTCCTCATCATGACACCTTCTACCTCTTCATCTACCAAAGGACTTTAGGTCAGGTGGAAAACTTACCTT
TCACATCAAGGAAGGGAGATAGGGCTGGGGCAGGCGAGAATGAGCTTGATCCTTCTGCCCTGCCAGGCTTCTAGGGACCTTTTGTGT
GTGTGTGTGACTTGTTAACATTTTTTTGTTCTGAGATTTCACTTGTCTGTGGCATAGATTTTATTATTAGTAACTGATCTAATACTG
CATGAAAAGGCTTGTTTTAAAATTAATCATATGATTACTACTGCCTTTTTTTTTTTTTGAGACGGAGTCTCACTCTGTTGCCCACGC
TGGTGTGCAATGGCATGATCTCAGCTGACTGCAGCCTCTATCTCCCAGGGTCAAGCAATTCTCCTGCCTCAGTCTCCTGAGTAGCTG
GGATTATAAGTGCCCACCACCACGCCAGGCTAATTTTTTGTATTTTTAGTAGAGACAGGGTTTCACCATGTAGGCCAGGCTGGTCTC
AAACTCCTGACCTCAAATGATCTACCTGCCTCGGCCTCCCAAAGTGCTGGGATTATAGGCGTGAGCCACCGCGCCTGGCCGATTACT
ACTACCTTTAAAACAATGTTTTCTATCTGAGGATGTTGGGATAAAATCCACTCTAGGTATACATTACAACCAAGGGCAACCCCTCTA
GACTAGAATGCTGGAGGTTATATTTGCAAAGAGGGCTGTTTCCTTTTCTTAGGAATTTTGATCATTTGGACAGGATTCCTTCTTGAA
AGGAATCAGCACTGAGGCCCTTATATGGAATATTTCCATCACTCCCTAGGGTTGGCCTGTGCCCTTTCACAGTTCCTTCCATCCTCC
ATGCCTGGCTGTGGGCAACCCCTGATCTGCCTTCTGTCACTATAGATTAGTGGCATTTCAAGAGGAGCATTTATATATTTTTAAAAA
GATTCCTCAGAGTTTTAACTCCAAAAGCTGCCAAGAAAATTAGATTCTGCTAATTGCATCATCAGCAAACACTGAAATCTGTTCATT
CAATATTGGCTTCCTTCTGTATTCAATGAACTGTCTTTTCTTTAAAAAATATCTTTAAGAAATAGTTTGGTACCTGCAAAACTTTGA
TCACTTTTTTTCTTTTTTAACAGGTGGTACTTTGCATATCAGTTGATGTATCTCAAGACTATAACCAAGTAGAGAATGTCATAAAAC
AAGTAAGAGGTTGGGCTAATTGGAATTCACCAATGTGCTTGACTTTTGATTTGTTCTTGCCATTTTGCTATGTGGCTTGGAATGGAC
ACAACTTCTCTGTAGGTTTAATAGTTTTGTAAATTTTATTTATCAGCATGGATTTGCTGCTGAGTGGTTTTTCTTCTCTGTTTCTTA
ATGCCTGCTCTGCTACATTTTTCTAGGCACAGGAGAAACTGGGTCCAGTGGACATGCTGGTAAATTGTGCAGGAATGGCAGTGTCAG
GAAAATTTGAAGATCTTGAAGTTAGTACCTTTGAAGTAAGTAAAAATGTTTGCTCACTGCAGTAGCACATATGCTAAAAGCAGAGCA
AGCTACGGGTAAAAGCTTTAAGAATCAATTAGATGAATGCATTCAGATGTCCATGGAAGCTTTAGAAGACTCTAGAGGAAGGCTGAA
CAAGGTGAAGCTGAACAAGGTTTTCATCCCAGTGTCAGGACCATTGAAGCTTAAGAGTTTAGACATTTTACAGCTGAGAAAACCAGC
TAAAATGGGAATGGACCTTCTAATCTGACACAGATTACTGTAGCAGTTCACTGAGGTCATATTTTCAAGAGAACACTTTGTAACCTA
AGAGATGCCGGATGTTCCTATCAAACATCAGTATTATTAACAAGGAGAAACCAAAAAAGTTGAAGGACAGACTCACTAGAGTTCTCA
AATCAAGATAAAAAGATACTGCATCTTATTGATGTTTCTATTTTTAATCAGGTTTACCAAGGAACTTCTCTTGGTAATTTATTTCAG
CACCAAAGAAATGAACACTAGATTTGAAATTTTTATTTTTTCTTTTTTTGAGATGGAATTTCACTCTGTCACTCAGGCTGGAGTGCA
GTGGCGTGATCTCAGCTCACTGCAACCTCCACCTCTCAAGTTCAAGTGATTCTCCTGCTTCAGCCTCCCTAGTAGCTGGGATTACAG
GCACATGTCACCACACCCAGCTAATTTTTTTTATTTTAGTAGAGATGGGTTTACGCCACGTTGGCCCGGCTGGTCTCAAACTCCTGA
CCTCAGGTGACTGGCCCACCTCTACCTCCCAAAGTGCTGGGAGTACAGGCATGAGCCACCAGGCCCGGCCTGAAATGTTTTTAAAAT
GGAAAACGTTTTTTAGTTGCCATCGCATCAACAATCTTTTTCTTCCTTTTTTTTGAGACAGAGTCTTGCTCTGTCGCTGAGGCTGGA
GTACAGTGGCACGATCTCGGCTCACTGCAACCTCTGCCTCCCGGGCTCAAGCGGAGTGTCCTCCAGCCTCCTGTGAGGATGAAGCTG
GGGAGTGTGTTGGTGTCTTCGTGTGCGCCGTCTAGACAGACTTTCCCTTAAACCTCTCTCTTTATCTAGACTTACTATCCCTAGAAA
CAGAACCTCCAGTCTCCTGCCAGGCTGGAAGAGGGGTACAGATCTACCCTGTGGAGAGGGGAAAAACATCTAAGATCTAACTGCCTT
TTTTTTTCATTGTCCTACATTATTTTTTTAATTTGAAGTGAAATTCATATAACATAAAATTAACTGTTTTTAAAGTGAGCCATGCAG
AGGGATTCAGCATATTCACAATGAGGTGCACCCACCCCCTCCGCCTAGTTCTAAAGCATATGCATCACCCATAGGGCGGCCCTCACC
TGGTAAGCAGTTGCTCCCCATCCCTGCTCACCCCCACACCCAATCTCTGACAACCACCAATCTGTATTCTGTCTCTGGTCATTTCGT
ATGATTGGAATCATGATATGTGCACTTCTGTGTCTGACCTCTTTCTCTTAGCATCAGTTTTTGAGGCTCATCCACATGTAGCATATA
TGGGTGCCTCATTCCTTTTTATACGGTGGTGATATTCCTTTGTATGGATCGACCACATTCTGTTTAGCTATTCATCATCGATGGCCA
TTTGGGCTGTGTCTAGCTGACTGCTTATTAAATAGACTCTTAGTCCCTACTCCTGTCTCAGCCCCATGCCAGCCCACCCTTCCCTCC
ACCCCAGAGACTCTTGCAGCTGGAGCCTTTTGGGGTTCTGTAGTAGAATCTGGCTTTTTAGTTTCTCCACTCCCCGCTTAGCATTTA
GTTTTTTGTGGTCTGCTAAGTTAGTTATCCCTCCTCAACCTGTTCTTCAGCTTCCAAAAGGTTATTGCTGTTATTTTTATTCTTATT
TTCTTTGTTCTTGTGTGTTCTTGCCTTAAAAAACATCTCTTTACGGAGATTTTAGTAAGGTTTCTAAAGGGAGCCAAAGTAAACACA
GGCGTTCAGTGTGACGTCTTTTTTCAGATATTCTTTTAGAACTTTCTTTTTTTTTTGGAGACGGAGTCTTACTCTGTCACCCAGGCT
GGAGTGCAGTGGCTCAATCTCGGGTCACTGCAGCCTCCACTTCCCAGGCTCAAGCAATTCTCCTGCCTCAGCCTCCCGAGTAGCTGG
GATTACAGTTGCCCACCACTATGCCTGGCTAATTTTTTTTTTTTTTTTTTTGTATTTTTAGTAGAGTCGGGTTTTCACCATGTTGGT
CACGCTGGTCTCGAACTCCTGACCTCAGGTAATCCACCTACCTTGGCCTCCCAAAGTGCTGGGATTACAGGCATGAGCCACCGTGCC
CGGCTTAGAACATTTTTTATTATCAAAACAATATTATCTTACGAAAACACACTTTTGGAAAAAAAAGAGAATCCCAGCATCCTAACT
CAGTCACTTATGATCTTCTGTATTTCCTTGGTCTTCCCTCTGAGCATGTGTACTTTTACTTAACCTTTGAAGTAATTACAGTTACAG
CATATATACAGTCTAGCTTCTGCTTTTTAAACTTAAATAGCATAGATATTTTTCCCTGTCACTCCCAGTGTTTCTGGTCATCATTTA
AAACAATTATCTAGGCCAAGTACAGTGGCTCACACCTGTAATCCCAACACTTTGGGAGGCCGAGGCAGGCAGATCACTTGAGGTCAG
GAGTTCGAGACCAGCCTGCCCAACATGGCGAAACCCTATCTCTACTAAAAATACAAAAAATTAGCTAGGCATGGTGGCTGGCGCCTG
TAATCCCAGCTACTTGGGAGGCTGAGGCAGGAGAATCGCTTGAACCCAGGAGGCAGAGGTTACAGTGAGCCAAGATCACACCACTGT
ACTCCAGCCTGGGTGACAAGAGCAAAACTCCATTTCAAACAAATACAAACAAACAACAATAAAAAAATCTAATATTCTCTTGAATTA
AGATATTATGATTGTATGTTCATGTTCTGTTCATAATATGAAAAAAAATTATGATTGATCTCAAGTTCCACAATTGAGGTATTTAGT
TTTTATTTACATGATGATCTAATATTTTTACAAATATTTGAATATTTGAAACATTTTAATGCAAGTATTATTAAATTTTCGAGTTAA
AATTTAAGGTTTTTAAGAGCTCAGGCTAGGCGCAGTGGCTCATGCCTGTAATCCCATCACTTTGGGAGGTCAAGGCAGGAGTACTGC
TGGAGCCCAGGAGTTCAAGACCAGCCTAGGCAAAATAAAAGGACCCTGTTACTACAAAAAATACAAAAATTAGCTGGACGTGGTGGC
ATGTGCCTGTAGTCCCAGGTCCTCAAGAGGCTGAGGCAGGAGGATTGCTTGAGCCCAGGAGATTGAGACTGCAGTAAGCCAAGGTTG
CACCATTGCACTTCAGCCTGGGTGACAGAGTGAGACCCCGTCTCTAAAAAAAATGATAATAGTAAAAAAGCAAAAAATAAATAAATA
AAAATTAAAAGCTTCCAAGACAAGGAGAAAATACACTATTAGTACTCTTACCCATCATAATGGGAGAGCCAGTGGTTCTTGAGGTCC
AGGACTGTGCTAGCGCTTGTGTTTTCTCTTTTAATCTTTGCAACAGCCCTATGCGGTCATACCCTTGTTGACCTTGTTTACAGCCAA
GAAAATAAGTAGAATGTCTGACTTTCTTATTCATCATCAAATAGGCAATAAATGGGAGACCCAAGGCTTGAAACCAAAACTTTAAAT
CACCCTCCTACTCTGTGTTTTTCTTATAGCTTAGAACTAATAGGGCTACAAATACTTGGAACCTCTTTCCTAAAACTGTCTTTCAGA
AAGTACTACCCACAAACATGCAGTATACTTTTTATAAGCAGAAAACTAACTTTGACCAGTTTTCAGGAAAAACATTAACTGCTGTCA
TGGTCAGAATGCAAGACTAGACTGAAACAGTCTTCAAATGGTAGGTCCTGAGTCTTCAAATGCTAGGTCTCAAATCACATTTCATTT
ATATCATCATGTCATCATTCATTCATTCAGCAAGCCTGTTGGGCTTCCCAGTCACTTGAGCTTGCATTGAAGAACTGAAATCCAGAA
AGCAGACTAAATTCTCCATGTGCACAGCCTTGAGGCTTTTGACCTCATGTGGCCTCTTTGTTCCCTGCAGAGGTTAATGAGCATCAA
TTACCTGGGCAGCGTGTACCCCAGCCGGGCCGTGATCACCACCATGAAGGAGCGCCGGGTGGGCAGGATCGTGTTTGTGTCCTCCCA
GGCAGGACAGTTGGGATTATTCGGTTTCACAGCCTACTCTGCATCCAAGTTTGCCATAAGGGGATTGCCAGAAGCTTTGCAGATGGA
GGTAAAAGTTGTATTCATTTTTGTATTTATTCCTCTCCGCTCAGCAATATACATTCCGCTTTAGTTTTATAGCTTCTTTACATGTGT
TGTTGTATTAAATTAGAGATTTTTAGGTTTGACATTGGTGAAAGAGAATTGGCTCAACCCTGAGTTTTGCAGTTAACATTTTTCATT
GCATGAAGACTATAGTTAACGACAGATGCAAAATGGTAATGGGATTTTTGCTTGTTTAACTTAATACACAAAATATTAGTGATATCT
TCATAGTGTTATTAGTCTAATACTGTGTGAATGTGAGTAAGGTTAACCAGATATTTTATTTTATAGAGATTTTTCTTTAACAAGAAG
TTTGTCCTTATGACTGAATAATACATGAAATTAGTATATGTTTGAGGGAAGAGCTTGAATCAGCTGCTAATTTTTAGCTTTTAGACA
AGTCATTTAACTTGCTCTCAGTTTCCTCATCTGTAAAATGGGAGGGTTGGAATAAAGCAATAATTTGACTCTCCCAATATTTATATT
TTTATATCTTAAAGAATTGTTGGGAAAATGAATGCTTTTTATTTACATAGTTAATGGAACATTTTGTGGAAGAAGGATGTAGTTAGG
ATTTGCATCAAAATAGCGTCTGTCATGATGAGTGCAACTTTACACCTCAGCTGCTTGAAGCCGACTTCCACATTATTGTCACACAGG
AATGCTTAGAGAGCATCACATACGTATCTACCCAAAAATCACATGCTGAAAAAGGCAAGGGAAAGTTCTTTCTATTGCTGTGTACAG
TCATCCCCTCTTATCCGCAAGGGATGTGAGCTAAGACCCCCAGTGGATGCCTAAAATGTTAAGAGCCCAGAAGAGTATTCACTGTGG
CCAACCCTCTTATAGTAAGAGTGTGCTTTTGGGAATCCTGGTTGATGATTTAGATCAGGGGTTGACAGAACTTTCACCAAAGGGCAG
ACAGTATATATGTTAGGTTTTAAAGGCTGAATGGTGCCTAATACAGCTACTTAAGTAGGCTATGATACCAGGAAAGCAGCCTTACTA
GACAATGTGTAAATGAACAATTGTCACTGTGTTCCAATAAAACTTTACTTATAAAAAACAGGCAGAGGGCCAGATCTGGCTCATAGG
CCATAATTTGCTGACCCCTGTTTAGAAGACCCCATAAGAGACCTTGATGGTGTGTGATAGAGCACCACATTGGTGAGATATGGGACG
AGCGTTGCAGTACAGATGAGCTGCTGGGACAGCGACAGTCCTGATGTGCCGCGTAGGGACTGATAGATCATGCCATTCTCATACCAT
AGGAACCAGCCACAGATTTTATTGTTATCAACTGGCCTAATACTCTTAATAATATATCGTGATAATATGCTTAGCCAATAGGACACT
GATGTCTGTGAAATTTCAAACAAGAATACTGGTGTTTCTTTTGAAGTTTTTAACTTTGAAAAGTCCTTTTGTGAATAACTGTTTTAT
TTATTTTTATTTTATTATTTTTTTTTAGAGACAGCGTCTCTCTTTGTTGCCCAGGCTGGAGCGCAGTGGCACAATCATAGCTCACTG
CAGCCCTGAGCTCCTTGGCTCAAGTGATCCTCCTGTCTCAGCCTCCCAAGTAGCTAAGACCACAAGTGCACACTACCATGCCTGGCT
AATTTTTAAAATTTTTTGTAGAGAAGGGGTCTCACTGTGTTGCCAAAGCTGGTCTCAAACGCCTAAGCTCAAACAATCTTCTGGCTT
TAGCCTCCCAAAGTGCTGGGATTACAGGTGTGAGCCAGCACACCCAGCCACCATTTTATAAAGTTTTGTAAAGTATGTGAGGGAGAT
GTTGTGATGCCACCTGATGAGAGGAAAACCCAGTTTTCAGTAAATTCAGTGTGTTGCTCAAAGTCACATAGTTACTTAGTGACAGAA
ATCACAACACGATCCAAGCCTTGACCCCAGGTCTGCTGCCCTTTTGCAGCAGATGATGCATCCAGCTAGTTGCTTCACCTGGCTAGT
TGCCCCGGCCCTTTACTTTGGTTTTAGAATCCTTGTAGGTGAAGGGGTGGTTGCGGGATGAAGAATCTGCCCATTAGGGCTTTTTGC
AGAGCTGTGTCAGCATTCACTAATTTAGAGCAAAGTATATAATCTGGGTGGCCTGGTTTCACTTCAAGTAGAACAGAGATGCTAAAA
AAGAGGAAAAAGAAAGTGGATTGCTGGTACAGACAGAGCCCCCCAACCTCTGTGACAAGTGTGATTTGCATACTGCGAGTAGCCGGC
CTGCACGCTCAAGTCTGTATGACTTCTGTCCTTATTTGGACTCTATTAAAAATACCCTCACTTAAAAAAAAGAAAATAAAATGAACT
TATCCCATGAAAAATAGGTTCTGGGGAAAACGAACGCAGTCATCAAAATGTAGTTTGTTCGTAGTAGAACTTGCATCAAACCAATTA
TTGTGTCCCTACAGTAATTATAGTAGCATTTTTTTTGAAACCTGGATAATTTGGTCAAGGTAGGTGTCTTTTCTGTCAATGAAATCT
TATGTAATTGCTCAAACTCCAGATACTTTTTTTTTTTTTTTAATTTTTATTTTCTGAAGACAGAGTCTTGCTTTGTTGCCGAGGCTG
GAGTGCAGTGGTTTGATCTTGGCTCACTGTAGCCTCCACTTCCTGGGCCCAAGCAGTCCTCCCACCTCAGCCTCCCAAGTAGCTGGG
ACTACAAGTGTGTACTACCATGCCTCACCGTTTTTTTGTTTATTTTTTATTTATTTATTTTTTGTAGACATGGGGTCTCGCTCGCTA
TGTTGCCTGGGCTGGTCTTGACCTCCTGGACTCAGGTGATCCACCTGCCTTGGCCTCCCAAAGTTCTGGGATTACAGGAGGTGTGAG
CCACTGTGTCTGGCCTGTAAATATTTTTGACAATTCTTTGGTTTAATTAACTTTGAAGTCTCCCTGTCCACCTCCCTTTGCCCCCAA
ACTAAGGTAGGACCCTGGCATTGTTAAGGGTACTATGAGTAATTTTAAAAGAATGCTTTGTTGGCATTTTTTAGTCTTTTTTTTTTA
CTCAGTCTTTTAGGAAGAAAGGTAAAGATTTTAAAAGTTACTCTTTTGGAAAGCAGACTTCTGAGGTGGGTTATAGCTAAAGATGTG
GTAGCAGAGACCAAGCCCAAAAAAAACAAACTGATGCTCCCTTTTCTGCTTTTCTTACCCTGATCATGCCCCTCTCCTCCCATCTCT
TCCTTCTGTGGTCTGAAAGTAAGCTAAAGCTTGAATTCTGGAGTTTCATGTAAACAGCTGTGATCTTCTTTCCAGCATCCCCTGTCT
TTGTTTTGATGGAATAGGGAACAGCACATCCTATGTGGATTATTTTAATAACAAATCTGTTCTTTTTTTTTTTTTTTTTTTTTTTTG
AGATGGAGTCTCACTCTGTGGCCCAGGCTGGAGTGCAGTTGTGTGATCTCGGTTCACTGAAACCTCTGCCTCCCTGGTTCAAGCGAT
TCTCCTGGCTCAGCCTCCCAAGTGGGTGGGATTACAGGTGCCCACCACCATGCCCGGCTAATTTTTGTATTTTTAGTAGAGATGGGG
TTTCACCATGTTGGCCAGGCTGGTCTTGAACTCAGGTGATCTGCCTGCCTCGGCCTCCCAAAGTGCTGGGATTACATATGTGAGCCA
CTGCACCCGGCCCACAAATCGGTTCTTGAACAAGTGGTCAGTTGTCTTTCTGTTTGAGGTTGAGGCCAGGGAGAAGGGAAGAAGTAA
TAGTAGGATGGGTGTGGGGCAGAGTCGGGGGCACAGAGAATGGTCAGGCAACAGGGATGAGGAGAAGCAGTGGCCGGGCAGCAGGGG
ACTCAGTTAGTGAGGACCTGGCTCATCCATTCTTAGAAAGAGTGGTGATCTCAGGGAGCTAGCATTGTCTCATAAGAACAAGTAAAG
CCAGCCTACCCTCATTTTTTTTTATGACAGGGTTATGAGATTGGTAGATTAAAAGAAGATGTAGATGTGGTTTAGTATATTCAGATT
TTAGCAAATTATTTGACCAAGAGTTTCAGGAAACTAGAATCTTAACCTTATGCTTTGTTCTTAGAACGATACTGGCATGTAGAAGTA
ATTCAGTAAGTGCTCGTTGTTGCATGAATAAATAAAATAATATGTAAATGGTGAGGTGACTAGCCACTGGAGGTATCCAGCCAGAGA
CTGGGAGAGTCATTTGGTTGGATGGCATAGCGAGAATACTAATCTAGGAAGCGACTGGACTGGTTTCTCCCAACCCCAGCTATCTGT
GTTCCTTCCTCATGAGGAAGCTGTTCTGAAAAGCTCTGTTTTAACCTTTGGCAATGCAGCCGTAACCTTGAGTTTGAGAAGCACTGC
TCTGAAATCCTGTCTTCCATTAGCTTTGGAGCCAGCATCCTTCTTTACCATGAGAGAGAAGGCAGAGGGGCAGCACAGACGTGAGTT
TCTGGGCAGTCGATCTCAGTCTTTATGTGGGCTGCCTAATGACCACTCGTTGGATTTTTTGTGGTGTTTTTCTCTGGTCTATGCACA
GCAGAGGGCAAGGCTGGGGGAGACAAGGTTTTTATAAGTCACTATCTCAAAAACAAGTGGAAGCGAGTTTCTGGCTCATCTGCCTTA
CTACCTGGCCCCGATCCAGAGCTATTTATGAGCATGGCTGGGCCGTGAAGCTGCATGCCCTGGCAATGCCCGGTGGATGGGCTGTCC
CACACCCTAATTGTGCTGGAATGGAACCCACAAAGACATCTCACTTTCACGACTGCACTTTCCAGGATGGCTGGCCTGGATCTTCTG
CTTGCAGGCCTGTGCGTGCTTCTCCTTCCCCTTGAAGCTCCCCTTAAAGCCCTCAAAACTGTGCTTCACTGAAGGAACCAGGGCGAA
AAAGGGCTGCATAAGATGAGACTCTGGGCTACTGTTTTTATTTTTTTGTTGTTTTTTTGTTGTTTTTTTTTTTGGGATGGAGTCTCC
CTCTTGTCACCCAGGCTGGAGTACAGTGGCACGATCTCAACTCACTGCAACTTCCGCCTCCTGGGCTCAAGCAATTCTCCTGCCTCA
GCCTCCCAAGTAGTTGGGATTATAGGCACCCGCCACCACACCCAGCTAATTTTTGCATGTTTACTAAAGACAGGGTTTCACCATGTT
AGCCAGACTGGTCTCGAACTCCTGACCTCAGGTGATCCACCCCCTCCCCTCGGCCTCCCAAAATGTTGGGATTACAGGTGTGAGCCA
CTGCCCCCGGCCCCTGGGCTACTCTTAAAGGCCGTCACATTAGGCAGTGGTGTTGATTTTTAGCCTAGGGTACCCTTCTGAAGATGT
GCAGTGGCTGATCTATCAGCTGCCTCTCCACTTCCAGATCCCTTAGGTGTTTCCTAGGGACAAAAAAGTGAGCCAATACCAAATAAA
AATCATGGCAAAGAGATTTTCATGGTAGGAACCATTGCTCTTAGAGTTGCTAATTAGCAGGGCAAGATAAGCATGTGCAGCAGGGCT
GTGGCTGAGCAGTCTGTGGGAGGGGGTGTCCTGGCCAACAGCAGAAGAATCTGCTGGAATTGTTGGCGGTGTGTGTATGAGTGGGGT
CGTGAAGCGTGAAGTGTGCAGGCATGGTGGTGAGGCACACAAGTGTAACAACCTGCTCCAAGTGTCAGATTTAGAGGAGCCACATAG
AGTTTCAGATAAGGCAGAGGGCTTTCCCTATGTGAATGCAGCTTGAGGTTGTCCACAGGGGAGGTACTTGGCTGGTCTGACTTGGAG
GTTCTACCCAGTGGTTCTTGTTGGTTCAGTGGAGGAGGAAGTGACTCTTGACCTCCTAATGTGCAGAGGCCGGTTATACAGGGAGCT
CTGGAATAAGCAGACTTCTCAGGGACAGTTCTGGACTGGGTTTGGAGTAGGGCACAAGAAAGGGATAGGGCAGCAGATTTTAATGAC
CCCAATGTATTTTGTGGATGAAATCAACACAAGAGTATCAGAAGCACTCTGAAGGAAAAACAGATGTACAGTCTGGTGAAGAAAGGA
GGGGCAGACTGGTCCCATTGCCTTGATAAAGGGCTTGGATGCTGTGCAGTGCGTGTGGGAACTGGACTCTTTTTAAAACACTGCCCT
CGTCGGACGGCTCCCAGGGCCTCTGTCTGGGCAGCAGGAGTGGAAGACACTGCAAAGGAGGTGACTTGCCATACTTTAGGGCCAGCT
GTTTTCTGGGGTCCAGGTTAGGGTGAGCTTTTTGCCTACATTCTGGAATCATCATGGAGAGGGGTTTCTCAATGCAATTGGTGTAGT
TCCCCGCCTAACAGAATCACCCCATTGTTCTCACCGCTTTTTGGGAATCAGGGCAGCCAGTGCAGGTTGGCAGTGTAGGACCTCAGT
GTGTCTTGTTTACTGAAGGTACACGTGTTGTTTTGAAATGCAGGTGAAGCCATATAATGTCTACATCACAGTTGCTTACCCACCAGA
CACAGACACACCTGGCTTTGCCGAAGAAAACAGAACAAAGGTCAGTATCCTACATGTCCCAATTTGAACCTCTAATGTTTACTGCTT
TTCTGATTTTCTTTCTACTCTTCAGCTCACACTCGTTTTCACCAATCAACAATGTTTGTTGGGTAGAGTGCCTAGCTGGTTTCTGAT
GACCTTGAGATTCTCCAGGCAAATATTCCTTCTTTCCTTCCTCTTTTCCCTCACTTTATTTTCTTAAAGTTTAAATTTCTACCAGAT
TTTTTCTCTATAGAGACAGGGCCTCACTCTGCCATCCAGGCTGGAGTGCAGTGGTACAATCGTGGCTAACTGCATCCTCAAACTCTC
AGGCTCAAGCAATCCTCCTGCCTCAGCCACCTGAGTAGCTGGGCCTACAGGCATGTGCCACCACACCTAGCTAATTTTTAATTTTTT
TGTAGAGATGGAGTCTCACTATGTCGCCCAGGCTGGTGTCAAACTCCTGGCCTCAAGTGATCCTCCCTCCTCAGCCTCCCAGGTGCT
GAGATTATAGGCATGAGCCATTGTACCCAGCCATTTCCAGCACATTTTTATCAAAGAAGTATGTCACACAGTTTTAGAGTCCAATAT
CAATCCTTTGCCTCATCTCTCCCATTCCCAATTCCTACTCCCTAGAGGTAGCCATTTATTTTAACTAACATGTTTATACTGCTTTTT
TTTTTTTTAAGATTTAGAAATTATTTATTGACTTTCTACCGTGGAAGATAAAATTTAAGACCAGGCCAGGCACAGTGTGGCTCATGC
CTGTAATCCCAGCACTTTGGGAGGCCTAGGCAAGAAGATCACTTGAGGCCAGGAGTTCAAGACCAGCCTGGGCAACATAACAAAACC
CTGTCTCTACAAAAAATTTAAAAATTAGCCAGGCATGGTGGCACATGCTTGTAGTTCCAGCTACTCAGGAGGCTGAGGTGGGAGGAT
CATTTGAGCCAGGGAGGTTGAGGCTGCAGTGAGCCATGATTGTAGTACTGCATTTCGACCTGGGTGACAGAGCGAGAACCAATATCT
AAAAAAAAAAAAAATTAAAATTAAAAATAAAAAAATAAAAATTTAAGGCCAGCCATGGTGTCTCACACCTGTAATCCCAGTACTTTG
GGAGGCCATGGCAGGAGGATTGATTGAGCCCAGGAGTTCGAGACCAGCCTAGGCAACATAGCAAGACCCTTTCCCTACAAAAAAATA
AGCAAAATTAGAATTGTTCCAGCTACTCAGGAGGCTAAGGTGGAAGGATAGTGTATTAGTCCGTTCTTGCATTACTATAAATAAATG
CCTGAGATTGGGTAATTTACACGTAAAAGAGGTTTAATTTGCTCACAGTTCCACAGGCTGTACAGTCTGCTGCTTCTGGGGAGGCCT
CAGGAAACTTACAGTCTTAGGTAAAGGGAAAGCAGGTACGTCTTACGTGGTCGGAGCAGGAGGAAGAGAGAGAGGGGAGGTGCCACA
CACTTTTAAACAACCAGATCTCGTGAGAACTCACTCAGTATCACGAGAATAGCAAGAGGGAAATCCACCCCCATGATCCAGTGACCT
CCCACCAGGTCCCTCCTCCAACATTGGGGATTGTAGTTGGACCTGAGATTTGCATGGGGACACAGATCCGAACCATATCAGATAGTT
TGAGCCTAGGACGTCGAGGCTGCAGTGAGCCAAGATTGCGCCACTGCACTCCAGCCTGGGCAACAGAGTAAGACCTTGTTTCAAAAC
AAAAACATTAAGACTCATATCTGTTCCCCATTCCCTGTGTACTCCTCCCATCTCTTTAGTTAAATCAGGTTTAGTGCTCATATTAAT
ATTATGCACACTTGGACCATTCTATAGTGACATTTCCTTTCTTGCCTTTTCATTTGCTTAGCTTTGTAATTATCTATAACTAATTCA
GTCCCAGATTCAAACAGAACTGAGATTCTCCTTTTGATCCATGTGAGCACATTGGACATTCTATTAGATTCATTCTTTTTTCCTCAG
ACATTTCTCCTGGGGTCGTCCCCGTTCCTGGTCTGTTCTAGTTCTGCCACCCAGCGGTGGTGTCAGGACTTGTCTTCACCTCACACT
GCACGTTCCCTTCTCCCCTCCCTTTGACGGAGCCCATGACTCTCACTTTTTTGGTTTATCTGCTCATTTTGGTGGAGCTATAATATT
TTCCACCAATTTTCTGAGAAAGGGCACATCAGAGGTAATATTGTTTTAGAAACAACATGTCTGAAAATGCCTTTATTTTTATTTTAA
CCTTTTTTTTTTTTTTTTTTGTGATAGAGTCTTGCTCTGTCACCCATGCTGGAGTGCAGTGGTGCAATGTTGGCTCACTGCAACCTC
TGCCTCCTGGGTTCAAGAGATTCTCGTGCCTCAGCCTCCTGAGTAGCTGGGATTACAGGTGCGTGCCACCAAGCCCGGCTGATTTTT
TGTATTTTTTTTTAGTAGAGACGGGGTTTCACCATGTTGGCCAGGCTGGTCTCCAACTCCTGGCCCACTCTGGCCTCCCAAAGTGCT
GGGATTACAGGCATAAGCCACTGTGCCTGGCCATTTTTCTTTATTCTTATGTATTGTTTTGTTAAGTACAGATTTCTAGTTTGGAAA
TAATGTTTCCTCAGATTTCTGTGAGCATTTATTGATTTCCAGCTTCCAGAATTTCTGTGGAATCCAGTGCTATTTTGATTCCCAACC
TTTTGTATAGAACCTGTTTTTCCATTTATTTTTGGAAACCTTGTGTCCCTTTTTATCCTTATTTAATGGATTTTGGCATGAAATCTT
CTCATTTCATATGAGCCCTTTTATTTTATCAACTTTTGTTCTGGGACATTTTATTATATTACTCCTTTTATCACTTCTTTCTTCCCA
AAGTATTTCAAGTGTTCATTCTCTCTGGAGCATTTATTATTCAGATTTTTGAACTTTCTGGACTGATCCTCTAATTTCCTTCTATTT
TCTCTCTCTTTTTTTTTTTAATCTCTTGGGCCTTTTGTTCTCCCTTCTAGGTTTTTATTCGCTTTATCCTTAAACCTTTTTGTTGAA
ATCTTTATTTCTACTGGGTTACATAAAAATTACTTTAGATCTCTTTTCTCTGAATATTTAAAACAGTTTTTAAAAATAGCTTCTTGC
CTTATTTCATGGATTTCATATTTTCTCTTATGTCTAAGGATATTCACTCTGGATGTTTAGCCTTGTTTTTTTGTTGCTGTTGTTTTG
TTTGAAAATTTCTTCTATATCTTGTATTATTTATTTCCTCCTACTTTCTTTTTCTCTGTTGTTGTTTTTTCCGGCTTCCTGAAATGT
CTGTTGATCCTTGATTGTCCATATTTAAGAGTGATGCACTAAAATGCCAATTGTAAATCTCTAGCTTCTCAGCTGTTTACTATAGGA
TGATCAGGAGGGGATGCAGCCACTTCATTAGAAGATTCTCATATGCCAGCATCTGCTCACTTTTCTCATGGTTTGATCAGTTTCCCT
ACTGAGGAATCATGTCTTCTTCTGCCTTTGGGAGAGTAAGACTTGCTGTCTGTGTTGTGGGAGGCAAGCAGGGCAGGAGGGCTGGGG
AGCTCACCACCTACGTGCCAACTTTTGCTTAATTTTTCTGTTTCTACAAAGTGCCTTATAACTACTCTTAGTGGTGAGAATTAAGAA
TCTGGTTAGTAAATTGCTAATCTTTTGCCATCGTAGAGGAGATGACTTGGGGTCTGATGATTTGAGTCTCCATTGCACTTATCACAG
CTTATAACAGAAGCTTCTCATAGCACTTAAAACAGACATATCTTTTATATAAAATGACTGCCAGTCATCCTGAGCTGAATATAAGCT
TCACACTTTTTCAATGGAATATTCCCTTTGCCTTGACACATAGTAGTTGCTTTTTTGAGCAAATGAATAGGTGAAGCGGGAAGAAAG
GAATTAATCAGAACTTACCACCTTGAACTGTGGCCAATTTTAGTTTTATATATTTTTTTCTCTTTTATACGAATACTTTGTAAATGT
GAATCTCTGCAGCATAAAAATTGGCACTTAAACTCCCTAGACTGTATATTTGTAGAAAGATGTTTGTGATTTAGGTTTTTTCCCCCT
ATTCTTTTCTCTTTGGCTCTGCCTTTTTAAAAATTACTGCTATTTCAAAGAAGTAAATTTAAGCCAAATGTCCTGTCTTAGAATAAG
CAGAGATCTTCTAAGCAGCTTTGCAACATTCGGTATGCAGTCTTTTAGTTTGAAGCCAGAGCTGACAAAGGGTGGTGATTTCCCTGT
TAAGGTTTGATTCTGATGTTGATTAAGTTGGGGTTTTTTCAGTAAAATTTTGGGCAGACTTGTTGGGGGTAAATACAATTCTACATT
GATGTGTGGGCCCTTTAGGGGGCGCCATAGTACTTTAGAATCTTATGTGTATTTAGTGTGGTTATGCCAGCCTGGGGGTCTGGAAGA
ATGAATCGTGGCTCCTTCAAAACAGCCTTTCTCTTAAGAAAGGTTCATAAGAAATACTTGATCTTTATGAAATACTTTACTTTCCTA
TTTTAAGAAAATATAATCTGGTTTTTATTTTCTTTAATTCCTTCAAGCTTACGTGGTAAAGTTTACAGTCTCATGCAGTTTAAGGCT
TTTGTTTGACACTTCTACTTGAAATTATTTGACACTTCTAATTGAAACTAACTGAAATAATTTTTGAGCACACATATGCAAGACATC
GCAAAGAAAAGCCAAAGATAAATAATTTCTTACAAAAAAGGCCCTGCCCACAAGAAACTTAGGAGGTAAAATGCAAATGCACAAGTA
GCTATAATGTGTATTAAAACAGAAATGTTCACAGAAGAAAGAGAGAGGTTACTGCCAGTTAGGTATTTAGGGAAGGCTTTTTGAGTT
AGGTAAGAGTTGAGATGGACCTTTTGAGGAAGAAAAGGCTAAGATTTGGGGTCTGCAGCTCCAGCCTGGTAACTAGCATGCAGCCCA
TCTAGGGACCACTCAGGGTTATCTCCTAAGTGGCTCCTTCTGTTTCAGTGTCCTGTGCATTTGCCTCCTCAGCAAGTCCTTTAAGAA
GCATCTGCAGTTGTTGAATGGGGGCTGGGTTGCGACTGTCCCTGCCTTGGGCGCCAGGTTCATCCCCAGGGATCAGCCCTCTGCTGA
CCTGGGAATATGGTCAGAATGGAGTTGCTGGTCTTGGGAGCAGGCTTGCTCTGGACACAGGTCTAGCCTCTACAGAAAAGCACCATC
ATTCAGGTGTCCGCCCAGCACTGAGGAGTCAAAGGGGGTCTTTCTGAACTTGTATCCAGTGATGTGTTCTCACTCTAGCTTTGATGG
TCCTCAGTGGAGCCAAGACTCCGTATGTTCTAAGAGAATGGCAAAGGCCTCCTTGTCTTGTGTTAGCTACTGGGTAGAATTTTACGT
AGTAACTAGGTAGACACTGATTTAGAATCCAGATGGACCTTATTCTAGAAATAGGAATCTGCCCAAAGGTAGAAAATTGGAATTATT
TGTAAATCAGGAGGCTGTAAGAATAATTTATTTTTAATGTAATGGGCAATCATGATATTGTTATGTACATGTTTTTCCACTTTTAGC
CTTTGGAGACTCGACTTATTTCAGAGACCACATCTGTGTGCAAACCAGAACAGGTGGCCAAACAAATTGTTAAAGATGCCATAGTAA
GTAAAATCCTTGTTTTCTGTACTATTTGTGTTTGTAGTACTATATTTCTGAAAAGATATTTGTCCAAGGACTTACTTCAAAACATCA
ATTATTAGAATTTTTTAGTCTGTCAGATTTGTCATATCTAATATTGGACTCAACTTTTCTTCTTTTAGACGCTTTATTGACTCCATA
TTAGTGAGTAGCCTCCTGAAAACTGATTGTATACCTTAATCTGAGGAACAGGTTTACTAGAAGGATGTCTGGAAACACCGGTTCTTT
AAAAAGAAAATGAGACTTTCCTATGAGTGTAAACGATATATACTATGTCCCATATACACCTGGCTGCTTTCTCTCCCATTCCTGCTT
CCCAGTATCATTTTCCCAAAAGTTAGCTAACGAGAAAGTGCCACCAGCCCCGTGGGCCTGTCCTCCTGTCTTCCCTGTTGATATGTT
TTTTAATTCAGAATCAAAAATGGTATTTTAAGAAATGATGTGCTGTTTTGTAGAAGAACAACATATGTGCCTCATGCTTTTTGACAA
CATTTCTGTTAATGCGGAAAAATGCACATTTATGAGGGATGTCTGTAAACCACAGACCCACACTGAATTTGGAAAATCTATAGGACC
ATTGGAATGAGAGGTGTCAGCACATGGTGGACAGCCTTGAAGTGGCCTAAATGCTGTCACATTAGACTGAACTGAGCAGAATGAGAA
TAGTGAATTCTGTTGAAATATATACATGTGTGAAGAACTTTGTCCTGAGTGCATTAAAAGGCTCAGGAAGATGGCACAAGTGAGACT
TTATTTTTCCTGCTTATCTCGGGTCAAAGCAAATGCATTTGGTGTTTTTTGTTGTTGTTGTTGTTTTGTTTGTTTGTTTGTTTTGAG
ACGGAGTTTTGCTCTTGTTGCCCAGGCTGGAGTGCAGTGGCACGATCTTGGCTCACTGCAACCTCTGCCTCCTGGGTTTAAGTGATT
CTCCTGCCTCGGCCTTCTGAGTAGCTGGGATTACAGGTGCTCACAACCACACCTGGCTAATTTTTTGTATTTGTAGTAGAAATGGGG
CTTCACCATGTTGGCCAGGCTGGTCTCGAACTCCTGATTTCGGGTAATCCGCACGCCTCAGCCTCTTGGCCAAAGTGCTGGGATTAC
AGGCGTGAGCTCCGTGCCCAGCCCATTCAGTGTTTCTAATGATGCTAAATAGCTCTTAGTATTTTGGCAGTATTCATTTAGTTTAGT
GGTTTTCAAACTTTTCTTCTCTTGACCACTTTACATTCTTACAAATTGTCAACAACCCCAGAGAACTTTTGTTTATTTACATGGCTA
TATTTATTGATATTTGCCATATTTGAAATGGAAGCTGTTTGTAATATTTATTGATTCATTAAAAATAACAAAAATGGCTGGGCACAG
TGACTCGTGCCTGTAATCCCAACACTTTGGGAGGCCAAGGCGGGCGGATCACCTGAAGTCAGGAGTTCAATACCAGCCTGACCAATA
TGATGAAACCCCGTCTCTACTAAAAATACAAAAATTAGTCGGGTGTGGTGACATGCGCCTGTAATCCCAGCTACTTGGGAGGCTGAG
ACAGGAGAATCGCTTGAACCCAGGAGGTGGAGACTGCAGTGAGCTGAGATTGAGCCATTGTGCTCCAGCCTGGGCAACAAGAGCAAA
ACTCCATATATATATATATATATATATATTCACATATATATATATATAAAGTCACATATATATATATATAAAGTCACATATATATAA
AGTGACTTTTTTTAAACAAAATTTACTACTTTTCAAAACAAAAGTTCAGTGAGGACGGTGGCATTGGTTCACATTTTCACCAGTTGC
TTTAAGGTCTGCATTAACAGCTGGATTCAAATAGCTGCTTCTGCATTCCATCTGTTGTAATAATGTTGTTTTGATGGAAGGATATAC
AGAAAATCTGGCCTGAGACAGATACGGAGTTGGGAAAGGGAAGACTATTTTACTAGGCTTTTCAGATAATTATGGACATTCTTTTTT
GATATCAAACCCAAACTCAACAAATGGTAGTTTCTTAAAGGTTAGTTGCAGTATAGAATTGGAAACTACATCAATGAATGTTTTGTT
CTCTGTTACATTAAATTCCATTGGTCCGTCTTGTACTTTGAATGGATCTTTTAACCATTCATGATTTCGTAACATTATGCATTAGTC
ATTTGGAAAATATGGGTTGACTGAGTGATCAGAAAAATCGCTTTCATCAGTATTACTGCTGATCTTATCATAAAAGTATGCAGCTGA
CAGTCTCGTGATGGTGGATACTAGTTTTTCCAAATCTTAATTTTTTCTTGAAGGCTCAGATTTCACATTGATGACACGTGGCCAGTT
GTTTTTCCTGAAGTGACACACTTGCTTGCTTCGCTTTCTGCACATCTGCCACATACCTAAGTCTGAATTACTATAGATTTCCTGTCA
ATGGTCTTTCATATAAAAATTATGCTCTCCAGCCAGGCACGGTGGCTCATGCCTGTAATCCCAGCACTTTTGGAGGCCGAGGCGGCT
GGATCGCTTGAGCTCAGGGGTTTGAGACCAGCCTGGCCAACATGGTGAAACCCCATCTCTACTAAAAATACAAAAATTACCCGGGTG
TGGTGGTGCATGCCTGTAATCCCAGCTACTTGGGAGTCTGAGGCAGGAGAATCACTTGAACCTGGGAGGCAGAGGTTGCAGTGAGCA
GAGATCACACCACTGCATTCCAGCCTGGGCCACAGAGTGAGATTCTGTCGAAAATAAATAAATAAACAATACAAATGATGCTCCATG
AAAGAAGTGGCAGTTTAGCCCTTGACTTCAGGAGCTGCACAAGGACTTTTGTTCCAGGCAGTTGTCCCGCCCTGGAATGCAGTGCTG
CGGAAGCGCGTTGCGCGCATTCTCATTTGTCACACAGAGTATTAAGAAGACATGTCCTCGAGGGTTGCCATTGAACAAAATGATGAA
TTTTCCTGCTTTATCAAGAGGATTTTAAAGCAAAACTGACACTTTGATTTTTAAAAGAATTTTAGAACTTGTATTTTCTTTGTGTAT
TATTTATTACTGCGAGTGCGTGTGGTAGTAGAGGTGACAATGACTCCTAGTACAGTTTGGCAAGAACCCCGTGGTTCTTGCCCATGT
CTCTGCAGTTTTGCAGAGGCAAAACTGTATTTTCCATACAACACACTGTGGAAAAGACAATGAATGTCTTAGTGTTATTAGGACAAT
CATTTGTCCTTGGGGGCCCGCAGGGGTCCGTGGACTCACTTGTTGAGAACTGCCATCTGCCAAGGCCGTGCGTCCATTTCTGAGTGC
TGATCAATGCTTTGTGACCATTTTATCTCACTTAAAGTGAACGAGCACACTCCACTGTTTCTGATGTGTCCGACTGATTTCTGTCCA
CATTACTAGGATTCCCTCTTTCTCTTCTTTTTCTTCTCTTTTAGCAAGGAAATTTCAACAGTTCCCTTGGCTCAGATGGGTACATGC
TCTCGGCCCTGACCTGTGGGATGGCTCCAGTAACTTCTATTACTGAGGGGCTCCAGCAGGTAAGCTCTGCCTAGGCTAGCAATCAGA
CAGGCCGATGGACAAGAGAAAAACTGCCTTTCATCAAACCTTGATGAGAACAGGTTAAGGGAAAACTAAGTAACTTTATTTGACAAT
GTATTTTTATGGAGCTATCAACTTTTCCTCTTCCTCCACCACGAATGAGTAAATAGGTTAAAGATATAACTTCAGGAATTTAGAATG
GCAAGAAGTCTTCAGTGCCGGGCCTTGCAGATAGAGAAATAAAACACCGTATCTGCTGTTGAGGTGTTAACCTGGATTTTCACCTAA
GAACCACTGCTCCAATGTGTTTTGAAAATGGAATACTCCTCTAGAGTAAGGGGTAGCCTTGCTTAAAAAGTACTGGGATATTAGACT
AGAAAACCTTTTCTAGGTGAATCAGGTCACACACAGCTAAATTTAGTGAGACACGAATCATGGATTAAACAGTTGCTTCGCTTATAG
AATGTATTATTTTGTCCAAGATACACAGTGTTTCATTGGTGTGCTAGGCACTGTACTTGCTCATGGGGAATCAGATGGAAGGCGACC
TCTCCAGCTTCAGGGGCATACAGTCAAGTGGAATGCACTGACCAAAAAGGTGATAGTAGTCCGGGCGCGGTGGCTCACGCCTGTAAT
CCCAGCACTTTGAGAGGCTGAGGTGGGTAGATCACTTGAGATCAGGAGTTCGAGACCAGCCTGGCCAAGATGGTGAAACTGTTTCTA
CTAAAAACACAAAAATTAGCTGGGCATGGTGGCGGACGCCTGTAATCCCAGCTACTCGGGAGGTTGAGGTAGGAGAATTACTTGAAC
CCGGGAGGTGGAGGTTGCAGTGAGCCGAGATCAAGCCACTGCACTCCAGCCTAGGCTACAAGGGCAAAACTCTGTCTCAAAAAAAAA
AAAAAAGATAGTAGTGCAGTGTAATGAAGTAGGGGTTACAATAGAGGGAATGCTCTGGAAAGACAAAGCCATGGGATAGTTGTTAAC
AAGTACTAAATTCTGTGCAGTCAACCACATTCAGACTTATATAAATGACTTAGTTGTTGGTTTTCTTCAGGGTCTTTCCCAAGCCCT
CTTTTCTTTTCATTCTGTAATGTACCTACTGTCTTTGCCATTCTCTTAGAGGTTGACACTGTGTTAGATGTAAGCTCTTTGAGGACC
AGGCCCACACCTCTTGTTTGCCGTTGTACCCCTAACAGCCAGCGCCTGGCACTTAGTAGGAACTCCAGAATCCCTGAGCTGAACAAA
TGCATGAATTCTTTTATGTTTTAATTTTTGGGCTGGCATGGTGGCTCACACCTGGCGTGTTGTCCTGGACTCCCCTGCTTCTGTCCC
TCACGTCAGAGATCATTGTCATCCTTGTCACTCAGTTCTTCACATTCTTCCATTCTCTGCAATTCTGGCAGTGCTTCTGGAGCCCAG
GCCTGGGTTGCCGCAGCAGGCCCCCTGCTTGAGTTACACACTTTTTGAGCCAGAGGTTGTAAACGATTCCCATGGGCCTGTAAAGCA
GTTTTGTTGGGTCCATCTGGTGTGAGTGTGTGTGTTTAAGATTTTAAAATAGTTTTAAAATTAGGTTTTATATAAAAGGTCCAGATT
TCAGACTTTTCTTGGAAAATCAGAAGGTTTAGCTGCACAGAGTCTGCATTCTTACCTGGCAGTCATCAGCAAGCACAGGTGGCAGCT
GCTCCTGAGGGAGGAGCACAGTCCCTGCAACCACTATGGTCTGCAACCCCCTGCTCAGGCCCCCTGGATGCGTTTGAGTGTACCCTC
ACATTTTCCACACCAGTTGTTTCTAAACTGCAACTATACATTGCATGAAAACATTCAATGACTCCTTGATACCATCAGGATAAAGCT
TTAAAAATTCCATTGCACCTGAGTATGGTGGCTCATGCCTATAATCTCAGCACTTTGGGAGGCCAAGTCAGGAGGATCGCTTGAGCT
CAGGAGTTTGAGATCAGCCTGGGCAACATAGTGAGACCCTGTCTCTACAAAAAAATGTAAAAGAAAAGAAAGCCAGGCGTGGTGGTG
TGTACCTGTATTCCTAGCAACTGGGAGACTTCTGCTTGAGTCCAGAAGTCTGAAGCTACAGTGAGCTGTGATCATACTCCTGCACTC
CAGCCTGGGTGACAGAGCAAGACCCTGTCTCTAAAAAGGATTTAAAAATTAAAAAAATTTCCATCGCATGCCATAAACCACCTGATC
TCCTACCCCACGTACCTTTCCAGGCTCTTTTTTTTCTTTCTTTCTTTACCCATCTTCTTCTTTTTGGAATCTTCTCTGTGTCCTGCC
AACTTCTAATCCTTCAGGTCTCACCTTAAATATTGTTTTTTCCGGAAGATCCAAGGACTGGCTTCAGTGCTCTGAGGGTGTAGGGAA
AAAAACAAAGGGATTTTTCTATTCTCTTTTTTTTTTTTTTTTTTTTTTGAGACGGAGTCTTGCTCTGTCACCCAGGCTGAAGTGCAG
TGGCACGATCTCAGCTCACTGCACGCTCTGCCTCCTGGGTTCACACCATTCTCCTGCCTCAGCCTCCCCAGCAGCTTGGACTACAGG
CGCACACCGCCACACGCGGCTAATTTTTTTGTATTTTTAGTAGAGATGGGGTTTCACCGTGTTAGCCAGGATGGTCTCAATCTCCTG
ACCTCGTGATCCACCCGCCTCGGCCTCCCAAAGTGCTGGGATTACAGGCGTGAGCCACCGCGCCCGGCCTGATTTTTCCATTCTCTT
ATGCACGGGGACTCATGCCTATGGTCCCAGCTGCTTGGGAGGATGAGGCAGCAGAATCCTTTGAGTTTGGGAGGTCAAAGCTATAGT
GAGCCGCGTTCATGCCACTGCACTCCATCCTGAGCAACAGGATATCAGTAATCACGGGTCTCAACCCTGAGGCAGTAGTTTCAATTT
TACTTGTCTTTCCAGCCTGAGAATAAGCTCATTGAGGGCAGGGACTGTGTCTCTCACCACAGTGTTCACAGCACCTAGTATAGGGTC
TCGCTTTCATGCTCAGAACATAAATTGCAAACCAAAAAAGCCTTGATTGTAGTTTGGCTATTTGAGATGCTGCACTGGGTCTTTTTA
AAGCTATTATAACCCTAAGACTGCAAGCAATATATATTTTATTCTGCTTATGGTTTGAATTTATTAATTGGACAAATATTTTGTTAT
GTATCTGGGCTGTCCAGCGGTGCAGAGCACACACATTCTGTTCTCACAGCTTACTGACATTTACAGAGTTTTCATTATATTCATCCT
TAGAGCAGACTAGAAGATTTGTCTTAGAAACTGTTTGTTAGAGACCTAGGTACCTTGAAATAGTCTTGTACCGTTGGATGCACTAAA
AGCTCTCTCTTTATCTTGCAGGTGGTCACCATGGGCCTTTTCCGCACTATTGCTTTGTTTTACCTTGGAAGTTTTGACAGCATAGTT
CGTCGCTGCATGATGCAGAGAGAAAAATCTGAAAATGCAGACAAAACTGCCTAATCTTCTTACCCCTTGGAAGAAGACTGTTTCCAA
ATAATTTGAACAGCTTGCTGCTAAATGGGACCCAATTTTTGGCCTATAGACACTTATGTATTGTTTTCGAATACGTCAGATTGGACC
AGTGCTCTTCAGGAATGTGGCTGCAAGCAAGGGGCTAGAAGTTCACCTCCTGACAGTATTATTAATACTATGCAAATATGGAATAGG
AGACCATTTGATTTTCTAGGCTTTGTGGTAGAGAGGTGAAGGTATGAGAATTAATAGCGTGTGAACAAAGTAAAGAACAGGATTCCA
GAATGATCATTAAATTTGTTTCTATTTATTCTTTTTTGCCCCCCTAGAGATTAAGTCCAGAAATGTACTTTCTGGCACATAAAGAAA
TCTTGAGGACTTTGTTTAAACCTTCCATAAAAAAACAATTTTCGGTTTCTCGGGTTCTCTCTCTCTCTCTCTCTGTCTCTCTGTCTC
TCTGTCTCTCTGTCTCTCTGTCTCTCTGTCTCTCTCTCTCTCTCTCTTTCTTTCTTTGTGTATTTTATTCAAGATGAGTTGGACCCA
TTGCCAGTGAGTCTGAATGTCACTGACAGCCCTGTGTTGTGCTCAGGACTCACTCTGCTGCTGGTGGAAACTCATGGCTTCTCTCTC
TCTTTGATCCCATAAAGCTACGAGGGGGACGGGAGAGGGCAGTGCAATGGGAAGTAAAGAGATATTTTCCAGTAGGAAAAGCAATGC
TTTCTTGTCTTTAGACTCAAATGCTTAGGGAACGTTTCATTTCTCATTCATGGGGAAAGGCAGCCTCCTTAAATGTTTTCTGAAGAG
CGGTAAAATCTAGAAGCTTAAGAATTTACAGTTCCTTCAATAACCATGATGACCTGAAGTTCACCTATCCCATTTTAGCATCTACTT
GTTTTTCCCATCTCTTCCTTTCCAATTTTGCTTATACTGCTGTAATATTTTTGTAAAAAAAAAAAAAAAGGAAAAAAAAGACCAGCT
AAAATTTTCGACTTGACTTTTTAACTTAACTCATGAATTAATTAAAGCAAATGAAAAAATTAAAAAGTGTGACTTTTTCTCGGAGCA
TATATGTAGCTTTTAGGAAAGGCTGATGATGGTATAAAGTTTGCTCATTAAGAAAAAAAGACAAGGCTGATTTTGAAGAGAGTTGCT
TTTGAAATAAAATGATCACCTGTTCTTTATGTGACTCTCCCACTGAACCTGCAGACATTATTTTTATACCACATGCTAAGGAAGCCC
ACTCATCTAACTCTGTAGCCCTG
HUMAN mRNA SEQUENCE:hR22-022.1 (Seq ID No: 7)
GGCTCCTCCTCTTCCCCTCCGCCGCGGCCCGCCCCGGCCCGCAAACCCAAACACTCCAGGCGCCCGCCCGCCGCGCGTGATTCTCGC
CTCGCCGCAGCCCAGCCCTGCGCGCCTTGCCCGGCGGCCCCCGCCCGGCCGCTCCGGGCCCCTGGCCCCGCGGAGCGATGCTGCTGC
TGGCTGCCGCCTTCCTCGTGGCCTTCGTGCTGCTGCTGTACATGGTGTCTCCGCTCATCAGCCCCAAGCCCCTCGCCCTGCCCGGGG
CGCATGTGGTGGTTACAGGAGGTTCCAGTGGCATCGGGAAGTGCATTGCTATCGAGTGCTATAAACAAGGAGCTTTTATAACTCTGG
TTGCACGAAATGAGGATAAGCTGCTGCAGGCAAAGAAAGAAATTGAAATGCACTCTATTAATGACAAACAGGTGGTACTTTGCATAT
CAGTTGATGTATCTCAAGACTATAACCAAGTAGAGAATGTCATAAAACAAGCACAGGAGAAACTGGGTCCAGTGGACATGCTGGTAA
ATTGTGCAGGAATGGCAGTGTCAGGAAAATTTGAAGATCTTGAAGTTAGTACCTTTGAAAGGTTAATGAGCATCAATTACCTGGGCA
GCGTGTACCCCAGCCGGGCCGTGATCACCACCATGAAGGAGCGCCGGGTGGGCAGGATCGTGTTTGTGTCCTCCCAGGCAGGACAGT
TGGGATTATTCGGTTTCACAGCCTACTCTGCATCCAAGTTTGCCATAAGGGGATTGGCAGAAGCTTTGCAGATGGAGGTGAAGCCAT
ATAATGTCTACATCACAGTTGCTTACCCACCAGACACAGACACACCTGGCTTTGCCGAAGAAAACAGAACAAAGCCTTTGGAGACTC
GACTTATTTCAGAGACCACATCTGTGTGCAAACCAGAACAGGTGGCCAAACAAATTGTTAAAGATGCCATACAAGGAAATTTCAACA
GTTCCCTTGGCTCAGATGGGTACATGCTCTCGGCCCTGACCTGTGGGATGGCTCCAGTAACTTCTATTACTGAGGGGCTCCAGCAGG
TGGTCACCATGGGCCTTTTCCGCACTATTGCTTTGTTTTACCTTGGAAGTTTTGACAGCATAGTTCGTCGCTGCATGATGCAGAGAG
AAAAATCTGAAAATGCAGACAAAACTGCCTAATCTTCTTACCCCTTGGAAGAAGACTGTTTCCAAATAATTTGAACAGCTTGCTGCT
AAATGGGACCCAATTTTTGGCCTATAGACACTTATGTATTGTTTTCGAATACGTCAGATTGGACCAGTGCTCTTCAGGAATGTGGCT
GCAAGCAAGGGGCTAGAAGTTCACCTCCTGACAGTATTATTAATACTATGCAAATATGGAATAGGAGACCATTTGATTTTCTAGGCT
TTGTGGTAGAGAGGTGAAGGTATGAGAATTAATAGCGTGTGAACAAAGTAAAGAACAGGATTCCAGAATGATCATTAAATTTGTTTC
TATTTATTCTTTTTTGCCCCCCTAGAGATTAAGTCCAGAAATGTACTTTCTGGCACATAAAGAAATCTTGAGGACTTTGTTTAAACC
TTCCATAAAAAAACAATTTTCGGTTTCTCGGTCTCTGTCTCTCTGTCTCTCTGTCTCTCTGTCTCTCTGTCTCTCTGTCTCTCTCTC
TCTCTCTCTTTCTTTCTTTGTGTATTTTATTCAAGATGAGTTGGACCCATTGCCAGTGAGTCTGAATGTCACTGACAGCCCTGTGTT
GTGCTCAGGACTCACTCTGCTGCTGGTGGAAACTCATGGCTTCTCTCTCTCTTTGATCCCATAAAGCTACGACGGGGACGGGAGAGG
GCAGTGCAATGGGAAGTAAAGAGATATTTTCCAGTAGGAAAAGCAATGCTTTCTTGTCTTTAGACTCAAATGCTTAGGGAACGTTTC
ATTTCTCATTCATGGGGAAAGGCAGCCTCCTTAAATGTTTTCTGAAGAGCGGTAAAATCTAGAAGCTTAAGAATTTACAGTTCCTTC
AATAACCATGATGACCTGAAGTTCACCTATCCCATTTTAGCATCTACTTGTTTTTCCCATCTCTTCCTTTCCAATTTTGCTTATACT
GCTGTAATATTTTTGTAAAAAAAAAAAAAAAGGAAAAAAAAGACCAGCTAAAATTTTCGACTTGACTTTTTAACTTAACTCATGAAT
TAATTAAAGCAAATGAAAAAATTAAAAAGTGTGACTTTTTCTCGGAGCATATATGTAGCTTTTAGGAAAGGCTGATGATGGTATAAA
GTTTGCTCATTAAGAAAAAAAGACAAGGCTGATTTTGAAGAGAGTTGCTTTTGAAATAAAATGATCACCTGTTCTTTATGTGACTCT
CCCACTGAACCTGCAGACATTATTTTTATACCACATGCTAAGGAAGCCCACTCATCTAACTCTGTAGCCCTG
HUMAN mRNA SEQUENCE:hR22-022.2 (Seq ID No: 9)
GGCTCCTCCTCTTCCCCTCCGCCGCGGCCCGCCCCGGCCCGCAAACCCAAACACTCCAGGCGCCCGCCCGCCGCGCGTGATTCTCGC
CTCGCCGCAGCCCAGCCCTGCGCGCCTTGCCCGGCGGCCCCCGCCCGGCCGCTCCGGGCCCCTGGCCCCGCGGAGCGATGCTGCTGC
TGGCTGCCGCCTTCCTCGTGGCCTTCGTGCTGCTGCTGTACATGGTGTCTCCGCTCATCAGCCCCAAGCCCCTCGCCCTGCCCGGGG
CGCATGTGGTGGTTACAGGAGGTTCCAGTGGCATCGGGAAGTGCATTGCTATCGAGTGCTATAAACAAGGAGCTTTTATAACTCTGG
TTGCACGAAATGAGGATAAGCTGCTGCAGGCAAAGAAAGAAATTGAAATGCACTCTATTAATGACAAACAGGTGGTACTTTGCATAT
CAGTTGATGTATCTCAAGACTATAACCAAGTAGAGAATGTCATAAAACAAGCACAGGAGAAACTGGGTCCAGTGGACATGCTGGTAA
ATTGTGCAGGAATGGCAGTGTCAGGAAAATTTGAAGATCTTGAAGTTAGTACCTTTGAAAGGTTAATGAGCATCAATTACCTGGGCA
GCGTGTACCCCAGCCGGGCCGTGATCACCACCATGAAGGAGCGCCGGGTGGGCAGGATCGTGTTTGTGTCCTCCCAGGCACGACAGT
TGGGATTATTCGGTTTCACAGCCTACTCTGCATCCAAGTTTGCCATAAGGGGATTGGCAGAAGCTTTGCAGATGGAGGTGAAGCCAT
ATAATGTCTACATCACAGTTGCTTACCCACCAGACACAGACACACCTGGCTTTGCCGAAGAAAACAGAACAAAGCCTTTGGAGACTC
GACTTATTTCAGAGACCACATCTGTGTGCAAACCAGAACACGTGGCCAAACAAATTGTTAAAGATGCCATACAAGGAAATTTCAACA
GTTCCCTTGGCTCAGATGGGTACATGCTCTCGGCCCTGACCTGTGGGATGGCTCCAGTAACTTCTATTACTGAGGGGCTCCAGCAGG
TGGTCACCATGGGCCTTTTCCGCACTATTGCTTTGTTTTACCTTGGAAGTTTTGACAGCATAGTTCGTCGCTGCATGATGCAGAGAG
AAAAATCTGAAAATGCAGACAAAACTGCCTAATCTTCTTACCCCTTGGAAGAACACTGTTTCCAAATAATTTGAACAGCTTGCTGCT
AAATGGGACCCAATTTTTGGCCTATAGACACTTATGTATTGTTTTCGAATACGTCAGATTGGACCAGTGCTCTTCAGGAATGTGGCT
GCAAGCAAGGGGCTAGAAGTTCACCTCCTGACAGTATTATTAATACTATGCAAATATGGAATAGGAGACCATTTGATTTTCTAGGCT
TTGTGGTAGAGAGGTGAACGTATGAGAATTAATAGCGTGTGAACAAAGTAAAGAACAGGATTCCAGAATGATCATTAAATTTGTTTC
TATTTATTCTTTTTTGCCCCCCTAGAGATTAAGTCCAGAAATGTACTTTCTGGCACATAAAGAAATCTTGAGGACTTTGTTTAAACC
TTCCATAAAAAAACAATTTTCGGTTTCTCGGGTTCTCTCTCTCTCTCTCTCTGTCTCTCTGTCTCTCTGTCTCTCTGTCTCTCTGTC
TCTCTGTCTCTCTCTCTCTCTCTCTTTCTTTCTTTGTGTATTTTATTCAAGATGAGTTGGACCCATTGCCAGTGAGTCTGAATGTCA
CTGACAGCCCTGTGTTGTGCTCAGGACTCACTCTGCTGCTGGTGGAAACTCATGGCTTCTCTCTCTCTTTGATCCCATAAAGCTACG
AGGGGGACGGGAGAGGGCAGTGCAATGGGAAGTAAAGAGATATTTTCCAGTAGGAAAAGCAATGCTTTCTTGTCTTTAGACTCAAAT
GCTTAGGGAACGTTTCATTTCTCATTCATGGGGAAAGGCAGCCTCCTTAAATGTTTTCTGAAGAGCGGTAAAATCTAGAAGCTTAAG
AATTTACAGTTCCTTCAATAACCATGATGACCTGAAGTTCACCTATCCCATTTTAGCATCTACTTGTTTTTCCCATCTCTTCCTTTC
CAATTTTGCTTATACTGCTGTAATATTTTTGTAAAAAAAAAAAAAAAGGAAAAAAAAGACCAGCTAAAATTTTCGACTTGACTTTTT
AACTTAACTCATGAATTAATTAAAGCAAATGAAAAAATTAAAAAGTGTGACTTTTTCTCGGAGCATATATGTAGCTTTTAGGAAAGG
CTGATGATGGTATAAAGTTTGCTCATTAAGAAAAAAAGACAAGGCTGATTTTGAAGAGAGTTGCTTTTGAAATAAAATGATCACCTG
TTCTTTATGTGACTCTCCCACTGAACCTGCAGACATTATTTTTATACCACATGCTAAGGAAGCCCACTCATCTAACTCTGTAGCCCT
G
HUMAN PROTEIN SEQUENCE:hP22-022.2 (Seq ID No: 10)
MLLLAAAFLVAFVLLLYMVSPLISPKPLALPGAHVVVTGGSSGIGKCIAIECYKQGAFITLVARNEDKLLQAKKEIEMHSINDKQVV
LCISVDVSQDYNQVENVIKQAQEKLGPVDMLVNCAGMAVSGKFEDLEVSTFERLMSINYLGSVYPSRAVITTMKERRVGRIVFVSSQ
AGQLGLFGFTAYSASKFAIRGLAEALQMEVKPYNVYITVAYPPDTDTPGFAEENRTKPLETRLISETTSVCKPEQVAKQIVKDAIQG
NFNSSLGSDGYMLSALTCGMAPVTSITEGLQQVVTMGLFRTIALFYLGSFDSIVRRCMMQREKSENADKTA*
```

TABLE 2

```
MOUSE GENOMIC SEQUENCE:mD27-007 (Seq ID No: 11)
CAAGTTACACTCAACTGTTTTAGAAGAGCAGTTCCCCAGATTTCTCCTTGGAGCTGTGAGTGACTACCATTGCGAGCAAGAGCAAGA
GGAAAGCACTACCTGTGAGCAGATGTCTGGTTCATTCTCACCCTGTGTGGTGTTCACACAGATGTGGCTGACTCTACTGGTTGTGAG
TGAGTAGATGGCTCAATATGGGGATAGAGTGGGTCGGACAGCATCTCTCTCCACTACAGGCCTGGTACTCTCTTAAGTTTTAGCTCC
TCAGTTTCCCCGTGTGAGGCTGTGTTCCCCGACTTTACTGTACAGCTTAGAATTTCCTCTCCTGAGCCCAAAGAAAGTAGTCACAGG
TTGAAAAAAAAAAATCCTTCAATGGACAGAGGAAGAAACGGTAGCAATGGCACTAGAGTTTCGCTTGCTTTCTTTTCTTTAACAAAT
ACTTTATGAAGCACAGAAAGTAAATCACACTTGCTTGTTCAAACTCTTTTATTCAAATGAGAACTATATTTTAATCCCGTGTAGACA
GGCTAGACCCTGGCCTTGTGCCTAAGATGATAGCTCCTTCTGCACATTAGCGAGAGCCATGAGCAGGACTGGGCGCCTTAAGTACAA
GAGGGGAAATCAGGAAGGAGAGGTACCTACAGCAGTGGCTATGTCACTGCCCATGGATATGCATGCATTTCTGTCAGTTCATGCCTA
CAAAGGGTTTGCTTCTTACTAAATTTTAAATGGAATTAAATGTGGGTTCATCTATTTTTAAAAAGTTCTTAGGCCGGGCAGTGGTGG
CGCATGCCTTTAATCCTAGCACTTGGGAGGCAGAGGCAGGAGGATTTCTGAGTTCGAGGCCAGCCTGGTCTACAGAGTGAGTTCCAG
CACAGCCAGGGCTATACAGAGAAACCCTGTCTTGAAAAACCAAAAAACAAAAACCAAACAAACAAACAAACAAAAGGTTCTTAGCGC
CATCTATGCACAGCATTGTATGCAATGCTGAGGACGGAGGGTTGTTTTGTGCCCACAAGGGAAAACACCAAAAAAAAAAAAAAAAAA
GCTCTCAGCAAAGCCAGTGCACAAGAAACAGCTGGGAAACACAATTCTAGTAAAATAAAATCCCAGTGGATGGCACAGACAGATAAC
CATAACCATCTACATACCAGTCAGGTGATCAGACTAAGCAACAGATAGGGCAAGGCCCATCAGAAACAGAAGCATATCCAAACTTAA
GAAGTACTTAAGAAGGCAGGGGAAAGGAGGGAAGAAGAAGGGAGGGGAGGACAGGACAGGACAGGAAGGTGTTCATACAGTGTGCTG
TGAGCTAAGGTTAGAGTTTGGATTCAAATCTGTGTTATCACCTACTAGCCACTTAACAAGCTTTCTTCTTTCTTTTCTTCCTTCCTT
CCTTCCTTCCTTCCTTCCTTCCTTCCTTCCTTTTTTCTTTATTGAAAATAGATTTTTTTCTTACATAATACATCCTGATTATTTCCC
TCCCTCTACTCTTCCCACTTTCTCCCCACCTCCCCTACCATGTGAATCCACTCTCTTTCTGACTCTCATTAGATAACAACGGGCTTC
TAAGAGATAAGAACAAAAAATATAATAAGTAAGATAATATAAAAACTATCACATCCAAGTTGAACAAAACAAACCAACAGAAAAAAA
GGAGTCTAAGAGCAGGCACAAGAATCAGCCCCACTCGTTCTTATGCTTAGGGCTCCCATTAAAAGCACAAACCTGGAAGCCATAATA
TATGCTCAGAGGACCTGGTGCAGATCCATGCAGGCCCTGCGCTTGCTGCATCAGTCTCTGTGAGTCCTGGTAACCTCTGCTTATGTT
GATTTAGAGGGCCTTATTTCCCGGTGTCCTCCGTCCCTACTGGTTCCCTTTCCTGAGCTCTGAGGGGAGAGNNNNNNNNNNNNNNNN
NNNNTCTCTCTCTCTCTCTCCCCCCCCCCCCCAATAATGTCAGGTCCTCTGGTTTCGTTCCCATCTACTGCAGGAGGAAACTTCTCT
GATGATGGCTGAACAATGATCTACAAGTGCAGCAGCATATCATTAGCAGACACTCACTACATTTCTATCTTGTTTTTTAACACTAGT
AATACTTGGTGCTTTCATTGAGACCTCCTCCATATATGTATATATTTTAGGAAGTTTCTACTGCATTAATTTTCCATACTAACCCTC
AAATACTCCTTAACTTTAGCTGTCTCTCCCTGAGTTCCCTCACTCAGTTCCCACCCCTCCCTCCCCATGAGATCCTCCCATCCCAGC
CTCTCTCCATCCATCCGTAATTATATATTCTGTTTCCCTTTTCTAATGAGATCTATCTGTTCCCTCTAGTCTCTCATCCTCTATACC
TACCTTCTGTGGTTCTAGGGATTGTAACTTGGTTATCATTAACTTAACAACTAATATTCACATATTAGTGACTATATACCATATCTG
TCTTTCTGGGTCTGAGTTACCTCACTCAGAATGATCTTTTCTAGTTCCATCCATTTGACTGCAAATCATAATGTAGGGTTTTTTTTT
GTTTTGGTTTGGTTTGGTTTGGTTTTTGTTTTTTGTTTTTAATGGCTGAGTTATACTCCATTGTGTAAATGTACCATATTTTCTTAA
TTCATTCTTCTGTCTGTTGAGAGACATCTGGGTAGTTTTCTGACTATTATGATAAGAGCAGCAGTGGACATAGTTGAGTCTTTGTGG
TACACTGAAGTGTCCTTTGAATATTCCCAAGAGGAGTATAACTGGATCTTACAGTAGATCCAGTCCCATCTTCCTGGGGAACTGCAA
CACTGATTTCCATAGTGTCTGTACAAGTTTGCAGTCCACCAGCAATGGAGGAGGAGTCTTCTGCTTATTAAATATCCTTGACAGCAT
GGACTGCCACTTGAGTTATTGATCTTAGCCATTCTGACAGGTATAAGATGAAATCTCAAAATAGTTTTGATTTTCATTTCCTCGTTG
GCTAAGGATGTTCGACATTTCTTTAAGTTCAATCAGCTTTCTTGTCTATGATATGGAACTTAAGTTAGAATGTTTCTGACAAGATTA
TATAGAATGACATGAAAATGCATACCAAATAGAAGCCCTCAAATACAAAAGCCATAATAACCATGATATGTAGGTAACAGTGACTAA
TTATTTGGTCTCAATAAAAATAAAATAAAATAAAATAAAATAAAATAAAATAAAACAAAACAAAACAAAATAAAATAAAATAAAATA
AAATAAAATAAAACTTGGATTTAAACTGTTCTTCTATCATTTCCAGCAGACAGGGACGATATGTCCCTCACTCACAGCTGGATTTTT
ATTGCATTTCCCAACAAATGGCTGGCATTCCAGAAATTTCTTTTAAGTGAGAGGATCAAATCCATCCTTCATCTAAATAATTCATAA
ATTAATAACCCTGCTGAGCTCTGCCTACTTCCTCGCCTCAATACTCGTGTCTCCAACCTGACCGATTTCTACTATTGCAACACTAGG
GTTTTGCCTTTCTGGGTTTTGGAAAGGTGTTTGTTTGTTTGTTTAATTGATTCATTACTCAAGAAAGCTTACAGCTCTCCCATTGCA
TAGCAGTCCTTACAGTCTAGACCAGGCTTTAGGAATGAGCAGCAAACACACACACAGTTCAGATAATCCTGGATAAACAACTGAAGG
AAGCTTGCCATTGCCACCACCCTGATGTGCTATTTCCTGTCTTCTCCATGGAAGACTTCTCTCTGCCGGAATACTTCATCCAAGCCT
TAAGTTCTGACTTCACCGCACTCCTTGGACTCTTTCTATAGTTCCCCGATGGCTTCTATAAGAAAAGAATGGCCCCCGCATTCTTCC
CCAACCATGTCTGTCCCACTACTCTTCACCCTTCAGAACTGCTCAGTCCACTTGGTGACACCAATGTCATCCCTTATTTCTGTGCCC
TTCTCACCTTAACCACCCTCTCTCTGTGTTCTCATGCCAGCACAGCGTCAACCTTCACTTGCATGATTCCTGTGTTCTTGGTCATAA
TTTCCTTCAAGTATCATCCATGGTATACCCACCTCCTCGGCGCGCGAGAGCGCGCGCACACACACACACACACACACACACACACAC
TGAATGTATTTTACTATTGTTACTTAATTCCCTCCAGGACTATACTTTTTTTTTTTTGTCAGAAAGTAACATTTTAAAATTTATCTT
TAAAGTTCATGGACAATGTCTTCACAAATATGAACATTTGACTAGCAATTGTCAATATTTTTATAAATGAATTTTTCATGAGAAGTA
ATTTGTAAAATGGGTACAGAAACATTATCTTTTTTTAATAGGAAAAAAATAAGGCCTTCCTCTGTGCCCTCATCTCTAACTCTTGGT
GTTTCTTTTCCAGCTCCTGTCAATGGACAGCATGGTGAGTCCTTTCTACTGCTTTGGGAGTGCTTGGAGAGTTTCTCCCTCATGCCA
ACATTTTTCTACTCAGGCTATCAGGGACTTGAAAGTGATGGCTGCCCCCTGGTATGGTTCCAAGCCAGAGCAAGTTCACACTGCCCT
CCAGCACTTGCTGGGTTAGGTGGTGAATGCACAGGCTGCCTGGGTCTTTCTGAGGCCCTGAATTGAATCCCATTGACAATGGACCCC
ACTTAGCAATGAAAAAAAAAATCATCTTTGTCTCTGCCTTAAATACAAAGGGCTCGGGATGGAATCCTTTCCAATCAGTCAACTAGG
AAAATTGTGAAACTTTCAAGATGCCAACAGTAACACTGTTTCCTTTGCTGTTTTAAGAGTAAAATTCTCCAGGAAGTGCAGGAGGAG
TAAGAGAGTATCCAACCGTTGGAGCCTGGAGAAGTGTAGAGGAATAGAACCAGAATGAAAATGTATGTGCCACACACACACACACAC
ACACACACACACACACATTTGTATATAGCACAGAACTTAAGTGTGTTTTAAAATAGCAACAGAAGCTAAATCACATCCAAGAAGCTG
AGAACTCATAAGACACACAGTCAGAGTAGGTCCTAGAATAGCTGTCTCACACTAGAGTGGGGTGACTAGTAATTTATTCAGTCTACA
AGGCAGTAGGCCCTAATCTGGCATTCAAAGTCTGGATGGTTCCTGGAGCCTCTGGTCCCAGTCCATGATGGAAGCTTGGAATGCTGG
TTCTGCTGTCAGTGGCAGAAGCAGAGGCAGCAGCTACAGGGCAGATCAACTCAAAGGGAAGGCCAGGAGAGCAAGGGTGGGATACGC
TCCTTCTGAAAAACTGTTCTATCTGGGCTGCTACCCAAGAGTGCTGCCCACAATCAAAATGGATCTTCTTACATCAATCAAGATAGT
CCATACAGCTCTTCAGGTAAAGTTCCCTACTCCAGTGATTGTGGCAGGTTAGCATGAAAGCAAAGAGAATTATCACACATTTTCTCA
GTCCGCCATCATTACTAATTTCCTGGGTCACAGGTGTCTTGGGTATTTGAGGATCCAAAGTAATATCGAGAAGTCTCTCCCTTCTCC
CCATAGCTAGCCACCCGGTCACTCGAGTGTCTAGTTCCCCTGGGGATCAGCAGGTGACTGTCCATTCCTTATTCTGACACAACTAAT
GACAGTGGGTTATGTAGCTATATCATCCTCTACGAAACAGCAGTTAAGTGCATGATCTCTCAAGCTTTTCTAGTGTAACGCCCGATG
GTAAAATGATTCACTACCTTAGGGGAAAATGCAAAGTTGTGATTGTAATGATTCTGAAATTCTTTGAGGGTTTCTGAATGACAAGAT
AGCAAAGGAAGAAGAAATGACTTTATCTAGGATCCATTCTCAGAGCAGGCTTACTAACCACACAGTGTTACAGTGCTAGAACTATCT
CCAGAAATTGTCTTGCTCCTTGCCATCCCTGGGCAATCACTCTTCCCTGTGGGTGGTGAGTGCTCCTAAATGATGCTTCTTCAGCCT
TCAATGACCCTCTGGGTGGGCAGGGGCAGGGCACGAATTGGGGGGGGGGGCACCGGTGACACAGGAGGACATGGAGGGGGGATGGAC
ATGAAGACAGGGGAGGGGGAAGGAACACTGTGTTCTTGGGAACTGTCCAAAGCCAGGTCCTTNNNNNNNNNNNNNNNNNNNNGGCTC
TTCTGGATTGGAAACTGTGGGAACATCTGCTACAGATTCATTTCTCTCCTCCTCAGAAGCTGCACAGCAGTCTGTGGTTTCCCTTCA
GCCTCCATGGACCACTTTCTTTCGAGGAGAGGTCGTCACACTGACTTGTTATAGATTCGGCTTCTCCGTACCCCAGAAAACAAAATG
GTACCAGAAAAGAAAAACAGTGAAGCAAACCCCAGGTGCTTTGGTAATTAAAGCACATACCTTAAAGGTCCATGAGTCCGGAGAGTA
TTGGTGCCAAGCCGACAGCTTACTTCCGAGCATGCACGTGAACGTAGAGTTTTCTGAAGGTGAGACAGAAGTAAAAGAACTGAAGCT
TCAGACACCGTTTCTGTCCAATTTGCCCTGTAGAGTTAATTATTACTTACCACAAACAACTCATGAACAAACCATTCTATTTCATGC
TATGGTTACAGAGAGATTAAAAGTGTCTATAGAGAACCAGATATGGCACAGAATTCATGTAAATTTAAACATAAAAATTAATAAAAT
TTAAGTAGTCTGAATAAAGTCTGCTCAGCCCAGTAGGCAGTGACTTTGACAAGTGCATACAGAAGGTCATAATTGTCACTCACTGCT
CACTGAGTTCTAGATCTAAATACATTCACTCCTATCCCTGTGGTCCTTAAAGTCTAGCACAAATGAGAAGGCTTGCTCAGCTTTGTG
ACCACACAGAAATTCAGGAGCTACTCAGGATTTACAAACTCAAATCTTGAGATTATTGAGCCCCAAGAATATGTACTGTTTATAAGA
TGCTCATGTAAATGAAATTTGAGAAGCAATTTACTTGCATACTTCTTGAATCCATTTTGTTTATTAGGAATAGTAAGTACCATAAGG
ACTTTCGTGTGTGTGTGTGTGTGTGTGTGTGTGTGTGTGTGTGTGTGTCTGTGTCTGTGTAAATATTAAATAGATAGAAATAAATGG
ATTGATTTTATAGGTACACAAAGAGAGGAAGGGGAGAAAGGGAGGGAGGCATTTTTGGTTTTGGTCTTGAAGCCTTCTGTTTTAAAG
TTGAAGTAGTGTCAGCCTTGTGAAAATAGTCTTTGATTTTGTGAGCAAGAAAACATACTATTAGCAAGCAAAAGAAAAAAGTAATAC
TCTTTGATCTCATGATTCTCTTGAACATTTACCTAACAAAGCACACAAATCTGCACACCCCAAAATAAAAGGCACTCCATTGAAGGG
AGACCCCATTAAATGATCTTGAGAGTATTGAGAGAAAACTGTGGCTTCTGAAAATTTAGCTGCAGTGATGGTTCAACTTGAAGGGGC
ACTAGACAAATGAAAGGCAAGCCGGCGTCATTTAAATCCCCTTAGAGTGACAGACATACAGAAACTCTGGTGTACTAACATGGAGAA
GGGACTGGAAGACTGTGTTCACAATGATGCAGGGAGCTTTCTAACTCGCCTTCTGCTGCCCAGCTTTTCTCAAGGTGCAGGTGTTTA
TTTTTAGTTCAGAAAAAGAATTCATGCTTTCTCCACTTCCGCTTCCTATAAAGCCTGTGTAGAAAGGCAACAACAAGAAGAACTGGG
AGGATGAAACAAGTGGTGTGCCTGATGTGTTCCACAGATTTTCTGGTGCTGCAAGCTCCACCTGCTGTGTTTGAAGGAGACTCTGTG
GTTCTGAGGTGCTACGCAAAGAAAGGCATAGAAGCAGAGACCCTGACATTTTACAAGGATGGTAAAGCTCTGACATTACATCATCAA
AGTGAGCTCTCTATTCATCATGCAAATCTGAAGGACAACGGTCAATACAAATGCACTTCGAAGAAGAAGTGGTCTTTTGGGTCCCTC
TATACTTCCAATACGGTCGGAGTTCAAGTCCAAGGTAGGGCTTTAATTATTCTGAACAGGTTAGTCAGAGACAGGCTTCCTGTGGAG
GTAGAATAGGAAGAAAGAGTGGGCTGCCAGTCCATTGAGCAGAAGGACAGTCTTTGGGTGGGAGGATGTGGGTAATCAAAACCAGCA
CCTCCCTCTAGCCAGCACTCGGGGAGCGGTACCACTCCACACCTCAGTGCTGAGGATGAAATGGGGTTTTCTGCAGACTACTTAACT
GCTCAGTGGATGATTGTGATCCTTTGTAGAGTTGTTCCCACGGCCTGTGCTGAGAGCCAGACCCTCCCATCCCATAGATGGAAGTCC
AGTGACCCTGACGTGTCAGACCCAGCTCTCTGCACAGAAGTCAGATGCCCGGCTCCAGTTCTGTTTCTTCAGAAACCTCCAGCTTCT
GGGGTCAGGCTGCAGCCGCTCCTCAGAGTTTCACATTCCTGCCATATGGACTGAAGAGTCAAGGAGATACCAGTGCAAGGCAGAAAC
AGTGAATTCCCAAGTTAGAAAACAAAGTACAGCGTTCATAATCCCAGTGCAGAGTGAGTATCATTCTGAGTTTTTTTCCAGGCATGG
AAGCTATTTAGAAGCATTCACCAGTTTGGGTCACTAGCCTCTATAAAAGGTATTTTTCTCAAACCTGTGGAAGTTGTATGGCTGTTG
TGGATAGCTAAACTGAGGAGCTTAGAACCAAGAAAGAAAAACAAACCAACAACAAAAACCATGTTCGTTTTTTCTCTCGATGCCTTT
AGGGAAAGGCTCTTCTGGTCTCAGCCTGACATGAGTTTTCCTTCCAACGCTATGATCCTAAACTGTAGACTACTAGGTGTTGCATGT
AGCTTTTGCTACTCACCATAACTCCAGGAATGGGCCACGCTACCAGCCCCCACCCAGCTTCCCTTGAATCCATAGATAAAACACACA
CACAGAGTTGTTCATTTTCAACTTGCCTTCTGGGCACAATTGCTGAGCATTATTAAACTCTCGCTGCTAACATGCCCTTATCAATTT
TCTTAACTCCGCACCTCCCACAAGCCCTAAACTTTAGTTGCTCTGTTAAATCTCGTCCCTACTAAACATCCCTGACCATTGCCTGTT
GGATAGCCACTTGCGTAGCCACTCTATTTTGAGATCTCACATGACCTGCCAACTTTTCTCCATCAGAAGCATGGTGAACTCTGTCTC
TCTTCCCTTGAATCTGTCTCTTTCCTCCGGGAACCTGGAAGTCCCCCTATTCCTTCTGCCCAGCAATTGGCCCATGGCCCCTTTATG
GACAGATCAAGAACCAACTGGGGAACAGGATAGCATCAGAACCATCCCTACAATGTGGTCAGCCCTCAGACTGGGCTGAATGTTCTG
TTCAGCCAAAAGGACAGGTGTCCAGAACATGAGGTGCCTGGTGCAGACTGGTTAAGGACAAGAAAGCCAAAAGATGAAGTCTTGCAG
AATAAATAGATCCATCTTGATGCCCTTAGAGATGGTCTTTCACAGACTAACACTTTTTCCTAAGGCTCAAAAAAAAAAAAAGTTTTC
TTAAGCGTGATCATTAGCCAGTGGCTTTGCCTGACTGAATTTGGCCTATCTCACAGGAAATCTAGTTGAGCATAGTAGGGGACACAT
AATCTAGAACACCTAGATCCTGGGTCTGGAGGGAAGGGAAGTGAGAGCATCACCTATGAATTTGTTCCCATTGCTAAGAAACCAATC
TGACTGACTAAATTCTATCAGTCTACTCTTTGGTTGTAAGCAATGGGTGCTCCTGTGGCTACTGATTTAAAGATGAACAGTTCAGAG
AGGAAAGAGAGGGCATTATCCATCAGTCACATCACAGTTATTCTTAGCACAGTCTTTCCTTCAACCTAATTGCTGAACCATCCCATC
ATCCCCATCCTCTAGTCAACTATCATAGTTTGTTCATTAAGTCTGGAAAATAATCCAAAAGGCACTGTCCTAAAACAGTATGCCTAA
AACCATTCCCAGGGTGGACAATCCTTCCTTGTGAGTCGATCCTGGACACTAGAGAAGTTAAGAAGTGCCAACTCAAGGCCAGTTCAT
TACTTATGACAATGCTTCATTTCCAGTCATACACGACTGGCACTGTACTCAGGACTATCATGTTTGATGCCAACTCTCTTGTTTCCT
TCCTCCACTTTTCCTAAGTGTTGAGCCATGGGTAGCAGCTCTGGAATTATCAGGATAGGGCAGACATGAGGTCAGGTTGGGCAGAAT
AACCTACAGTCCTGCTACATATCCTTCACAAGGCCCTCAAGGAATGTGGGGCCTTATCAGCTGGCAGAAAACACAGTCTCCATTGTC
TTCTCACTTCCCAGAGACAAGCCTAATGTTACTCTAGGGCCCCTCCCCACATGGTAAAGCAGGTTTTGTCTTCTTTCCACCCCTCAC
CCATTGCCAAAAGTCCCATGCATGTCATTGTTATTCATTTAGGAGCTTCTGCGAGATTCCAAACACACATCATCCCAGCCTCAAAGT
TGGTGTTTGAAGGGCAGTTGCTGTTACTCAACTGCTCAGTAAAAGGAGTCCCAGGACCCCTCAAATTCTCCTGGTATAAAAAGGACA
TGCTGAATGAAGAAACAAAGATTCTTAAGTCCTCCAACGCAGAATTCAAGATCTCCCAGGTGAACATCAGTGACGCAGGGGAGTATC
ACTGTGAAGCTACCAACAGCCGCCGAAGCTTTGTCAGCAGGGCATTTCCCATCACCATAAAAGGTGTATGTTACAATATGAACTAAC
CAGTACCTCCAGAGCTCATGTCTCTAGCTGTATATGTAGCAGAAGATGGCCTAGTCAGCCATTATTGGGAAGAGAGGCCCCTTGGTC
TTGCAAATTTTATATGCCCCAGTACAGGGGAACACCAGGGCCAAGAGGTGGGAGTGGGTGGGTAGGGAAGCAGGGGTTGGGGAGGGT
ATAGGGGACTTTCCAGGATAGCAAAATGTAAATGAAGAAAATACCTAATAAAAAATGTATAGTTAGGAGGTGTTAGTATATTCACAA
ATTGCCCAATTCTGTACTGTTTGCCTCTCAAACATGTATTTCCCTGGGACCTACAGTTGTTGACTTGTTTTTAAAATGAGTTGTTGC
TAATGCCATTAAAAAAAAAAAAAGTGTGTGTTGCCAGGTATATCTGAGGAATGGATCCCTCACATTGCACATGGGGATCCTTGAGGA
AGACTTGAAACAGTCAAAGTGGAAGTAGATGTTGTCAGTTACCAATAATTCAATGTTCTGAATAACTTGCCAAGTATTAGTCAAAGA
CTAATAGGAAAGACTTAGAAATAGATGGAATGGGCTGAGGGCAGTGGGAGCTCTCAGAACATCAGATGGAGGAGAAAACACTCAGTG
ACTAGGAACATAGCCTTTGTAAGTCCTCCTCAGTTAAAATGAAATGTCTCTTTATAAAGGAGGAACAGATGGGATGCCAGAAACAGT
ATATCTTGAGAGATGCCTCCTGGCATCTTACTGTAACCAGAAGAATTCTCAGGGACCCATCCTGGCCATGCTGCTATGTCTTCAAAC
CCTAGTAGCTCTCTTATTTCCACTCTTGGTCTCCTACAGTGAGATGCTATGGAGAATGTAGTAGAGCTTCACCAAGAGGCTGAGGGC
TTCTCCCTCTGACCCAACATCTGCCTGTTGGTTTTAACATGAGACTGAAATCCTCTGTTCACTGAGCACTTGCAAAATTATTCCCAT
GTCCAGGCCAGTGGAGCAAGGAGAAGCTATTGTCCACCAGAAGTAAGCAGCTCCAGAAGTCATATACAGGGTCAGGTTGCATGCTCC
TAACAGGCTACAGTTACCATCCACAGAGGCTCCTTACTTACCCCCCTCCTGCTCAGCTTCTAATATTCTGATAGAATGGATGGTTCT
GGGTACCTGGAAGAAGGCAGTACTTGAGGAGAGCATCCTGCAAGAGTATGTGAATTTAGCAGCTTAAAGGTCCTGGAGCAAAGCACG
GAAGTTACCCTAAGTAGAGAAATGGAGAGCAGAGACTGACACAGAGACTTAAAACTCGGGAGAGGAAATGGGCCACAGAGGAGCAAA
GAGTTGATTGAGAAAAGGGTATCAGTCCTATAGCAGCAAAATACAAACTTCTCCCCTGAGGGTACCATCTCTGTAAAATTGAGGAGA
GTTGGCTGACAAGGGGAGCCAGAAGGGATAAGGCAGATTGGAAAGGAACTGATGGAAGGCCATCATGGACGTCAGGAGATTGCTGAT
GTCCATCCAGCCTGGGAGGTTGAGCTGTCCCAAGATCTGGTAGAATGGAAACCAAGTATGTGCATTGGAACAAAACAAAAGTAACCT
GCAACCCACTTTCTTTCCTCCAGTCCCAGTATCTCAACCAGTTCTCACCCTAAGCACAGGCAAGACCCAGGCCCTTGAGGGAGACTT
GATGACACTTCATTGTCAATCCCAGAGGGGCTCTCCATGTATCCTGTATGAATTCTTCTATGAGAATGTCTCCCTGGGGAATAGCTC
TATACTCTCTGGAGGAGGAGCATACTTCAATTTCTCTATGAGCACAGAGCGATCTGGAAACTACTACTGCACAGCAGACAATGGCCT
GGGAGCCCAGTGCAGTGAAGCTATAAGGATCTCTATCTTTGGTAAGTCCTGGGTTCCTGCTGATACCCACCACTATACAGATTTCCC
TCTCACCAAACCCTTTTGGAAATTTGCCCCCACACCTTTACTTCCTACCATAATCACCTCAATCCTGTTCTAGGCTCCTATCTCAGT
CAAGCTCATTGTCTGTGCTTATCTACCTCCTCGCTAGGCCTGACTTTTGCCTAGCTCAGAAGCCTCCACAGCTCTTAATAAATGTGT
GTGGTCATCACACTCTGTCTTCTTATGCCCTGTAATGTTTAGTTCCCAAGCTAAGAGCATCAACAGGGGCACCCAGTTCTCTACAAG
CACCTATCATCTTCCCTACAGAACAGTGGCATGAACAAATACTATGCAAAACCTTAGTTTTTCCAAAGAGTTCCAAGCAATTACAAC
TCTCAGTGTAGGTTAGGAGGAGACTGGATTTGAAAGAAAAGGAGAAAATGGCATCCTTAGGAAATGTGTTCCTTATTTTCTAGGGTT
TTCATGAGCCTCTTTCTAGAAGCCCATTTAAGCAAATTTAAATTCACTGCATGATGTAGTTTTCTAGACCTTTGTTAGAGGGTGACA
GGATATGCGTCAGATCCAAAAGAATAGCTAACTCTGGTGCCTATTAGCATACCAGAGTGCATGTTGGCCTCTAGGCTCCCTCAGCAA
GGGCCTGTTACAGAGTCCTGCTCTGGCACCCTGGCTTCTCTCAGTCCTTCTCACCCCACCCCCTGCCCTAAACTTCTCCAACCCATG
AGTTTCCCTTGCCCCAGCTTCTCATTTCTACATATAATCCTGCCATTTGACCATGCACTCTCTTGGTTCTCTTTGCCCTTTTGGCTT
TTGGCTCTCTCTCAGTCAGCTTGCCCCTCTCTCTCTTTTGCCTCCCTCCCCCCAAACCTTGGTCTTCCTTTCTTGCTCCTCCTGCCC
TCCTCTCATGGCCCAGGTCACTCTCGTGGCCATATGCAGACCACTACTTTCTCTCCCTGCTCTGAACTCTTCCAGGTGTCTTTGGCT
GTACTCTCCCTCATAACTACAATCAATCGGTCAATCGATTGATCAATCACTTCTCCTCAGCCACACCTTAGATCAGTTATATTCTCC
TTTTGTTCAATATATAGATCATGGTTGGTCTCTGTCAAGGCCACTAGTTAACATAATGGAGCCACTGAAGGAACAAATGTGCTTGTA
TTTGCTCTTAGGAGAAAGTAAAAACCTAGCTGTTTTTTTGTTTTTGTTTTGTTTTTCAAAGCAGTAGAACAGCATTGTAGAAAAATA
TTGAGTCTAATACGTCAAACACATCTCCTTTGTATCACAAGGAAAAAGAAACCCAGGAGCCCTGGGTAGGTGCCAATTTTTAATACG
GCTGTTTTGAGAGTAAATAAATAAGAAAGACGACATAGGCAATTCAGGGAAGAACTAGAAGATTTCTCACAATGGGATATATTTCTC
AAAATAGAATTATTGGGTTTTGGGGGAGCGGTTGTAAAGTCTGAGGCTATGAAAGCTTACATAATGAGGATCTAAGAACGTGTTTTA
GAGTTTAAGTTAGATGTGCAAATGCGAGTTATAAAGGTTAGAGGATGTGAAAGCTTAAAGAGTGATAAGAAAGTGAGTTAAGGTATA
CAAAAGGAGGAAAAATGCTTCAGATTCCTTCCCCTTGATATGCTACTGCTGTATTAAGACTTCAAAATTCCAAAGTTTTGACATTGA
CGACTGGAGTTCTGCTAATATTGTAGACATAAGCTCAGAATTTTAAGTTTCTTTTGGTTGTCTGCTAAGTATAGATTTAAAAGTGCT
TCTCATTGTGCTAAATTTACACACACGCACACACACACACACACACACACACACACACACATCCAGTCTTCTGGATAGAGGAAAGAA
GCCACTCTTTCATGGTTTGCAATCATACTGAAAATCAAGATCAAGCAATCTTTTGCCATTTCTGCTCCATGGAAGGTTTCTGTTTTT
CTTGAGCTTGCCTTAAAGAGTGGTTATGCTGTTAACCCAAAACTATTTTCCAAGCTTCTACTATGACACAAAGATGTGAGTATGTGA
GTTTGAGTATGTGAGTATGTGAGTTTCAGTATAGACTACAAATAGTCGTAACACTAACATGTTTAAAACTTGTCTCTTTTTGTAAAC
TTAAAAAAAAAAAGATTGTAGTAAGCTTCAGGGGATCAACTTGAATCCACCTCTCATGGGTCAAATGGACTCCAGAGAAATACCCTT
ACCAATCAATCGCCTAAGGGGCCCAACCCATTACCTATTCCAGAAGGTGGGGTCCCTTACTCTTTCCCTGGTTTAGGGGCTCCTCTC
CCTCAACTACCAGGACCATGTCTCAAATGTATACAAGAAAAATTTCCCCCCAGACTCTTTAATTTTACATTCTGTCCTAATATGTGT
GTGTGTGTGTTCTAGCAGATTTCCAATCAATTAAAGACTATAAGCTGCTCTAACCACTGCCTGTACATCCCTAGCCCCAGATGTTCT
TGCAGAGCCTAGACACAACTTGTTCTTCCCAGACTTGACCAACATTCCAGCTTCATGCCCTTCCATCATTTGATAAAGATCAAATCT
TTAGCAGGAGCAGTCCTGACTGGGTCTCCAGCCTTCTACTCCATCACAGTTACTGGGCCTGTCAGACTGCTGCCCTACCCTCTGCAA
CAATTCAAATGGACCAACCTGTCTTCAGTCACTGCCCACACTGCCTGCCTCATAACCTATACGTAGTCCTCCCATATCGGATATGGC
CCCTTTCCACTGCAGGTTTTGGCATAGTCTCTGAAGCCCACTTCTTTCTCTTCTAGCAGTCCTCATCTCTCAGATGCCCAGCTTTCA
GCTGTGTCTTCATTTTCAGATTCCTCAAACATGTTTGTGTTTATGTGTCAGGTACTTCAGAATATATACAACACTATGCAAACATTC
TCACTTCCCACATTGGGATAAATAATAATTGAGCGCTGAAGAACCTGAAAAGGTCACTAAAACCTAATAATGACATTTTGCAGTCAG
AAATGCCTTTAGTCCCCTATACCCTCCCCCTGCCCAGCATTTGAAATGTAAATGAAGAAAATAGCTAATAAAAATTTTTTTAATTAA
AAAAAAAAAGAAATGCCTTTAGTGTGGAGGGCTGTGTTCAAATGGGAGAAACCGATGGCTGGTTCATCTCCCTCAGTTCCAGCGTCC
CACCCTACCCTAATCTTAGACGTTCCCAGGGGCCCAAGCTGTAGTGTAGTGGGGGGGGGGGGTGAAGCTAAACTGTGAGACCCTGGA
AGGCTCCCTTTCCTGATCTTGTACCATTTTCATCCTGAAGATGTCACCCTGGGGTGTAGGTTAGCCCTCTCGGAAATAGGAGAGTCC
TTCAACTTCTGTCTGACTGTGGAATGCTTAGGGAATGGGGTAGTTCTACTCCAGTGAGGCTGGCGATGTCCCAGGGCCCTAGCACAG
TGAGGCAGTGATGGTCAGTATCACAGAGACTTGTCATAAGCAAGCGCTGGCGGCACCTGCTCCTTCTGGCCGCAGGAAAGCTACTTC
CTGTTGGTATCTTCACAGCGCTCCTTTCATCTCTCATCCTCTCTAATCTTTTGGCTCTCTTCCTTTTATATTTTACCTCTTTCTTTC
TCTCAATGTCACCTCCTACTTTCTTGTCTTTGTTTTATTTTATTTTATTTACTTACCTTGCCCCACTGCCTTTAATTAGGGTTGTAT
GCATAAACAGGGGTTGGAGTCATTTACTTGAACTTGCCAGATGCTATATTCCTGAGGAGTAAGGCATAACGTGTTTTAATATCAGGA
TTGTCCTGTGTGTTTAGCTATAAAACCTTTATTTTTAAGCTTAAAATAAATTCTATGCATCTACCCAGTCTATTCGGTTGTCTACAG
GTTTTGTTGTGTTACAGAAACATTAGAACATAGAATTCTTGCATGAACTATATACATGTACTATATAATGTTATATATAGGCATATA
ATGTATTATCATACAATATATAACATATATTTGAAAATAAAATTTCCATACTATATGATTTTCCTCTCCCACATCCCCTTCATCCAT
CTCCATGCCCTTTCTCTCTCTCTCTCTCTCTCTCTCTCTCTCTCTCTCTCTCTCTAGAATACAATCAAACAAACATTTTTTAGAAAG
AATAGCAAATAAATAAGAAAAGGCACAAGAAACATAACACAAATCACACACACACACAACATAAAAGCACAAAACATAGAAAGTATA
ATACACAAGCAAAAAACCCAATAAGATTTAAAAAAATGCCCAAATAAAGCAATTTGAGTCAAAAGTGTACAAAAGCACCACTGAGTT
CTTTTCGTGTTGCTCATCTGGTGCTGGGCATGAGGCCTACCCGTGACTGTGGTCGTCTTGGGCACTGCTCTATTGCTCTGAAGGGAC
ACCATGACCAGGACAACTCTTATGAAAGAAAGTGTTTAATTGGGGCGACTGCTTACACTTTCAGAGGTTTAGTCCATTGTCATCATG
ATGGGAAACATGGTGGTAGGCAGGCAGGCTGGAAGACACAATTTGCTTGGTGTCACCCATCTCTTCAATCCTTACGATCTTTTTACT
TCCTCTTCCCCAACGCTCCCAGAGCCCTCAGTGGAATAATAAAGACATCCAACTTAGGAATGAGCATTCTAAAATCTCTCATTCTCT
GCACATTGGCCAGTTGCAGGCCTCTGTTCATTCCCATCTACTTCAAGAGGAAGCTTTTCTGATGGAGGCTGAGTGAAGCACTACTCT
ACAGATACAGCAGAATGTTGCTAGGAGTCATTTTATTGCTGTATTACTTTAGCAGAACAGTACTGTTTCTTTCCTCCTTGCCCCATG
TCCTATTTGCTTTCCTCTTAGTCTCAGGTTCTTGGCCACTAAAGCAGTGTCAGATATGGTTTTCATCTCATAGAATGGGCCTTAAAG
CCACTCAAATACTGGTTGATTACTCATCCAGTTTTTGTGTCATTATTGTATCAGTAAATCATGGAGGCAGGTCAATTTTGTAGATCA
CAGGGTTCATGGCCAGGTTGGTGGACACCTTTTTCCTCTGGAAGCACTGAATACACATTTTTTTACCTACTTGGTCCTCCAAGTAGA
TTGTGGGCTCCAAATTTATGCATTTCCTTAATTTTGTAGATATTGTCAAACTACCTTTCAAACGACTGTCCTCAGTTTCCTTTGCCA
CAATTTCATCAGTATTATCAATCTATGTAGGCTTGAAAAGGATAATAGTTTATGGCTGAATCTAATTGTACATTTAATTAATAATGA
AAATGAGTCTTTAATATATTTGTTGGCAGATGTTTCCAGTGATGCATATAACACTTTTCCCTTTTCTATTGAATATTTTTGTATATG
AAAAACTACTTGTGGATCAATGTCATTAGCTTTTGAAATCAGATGTTTTTTATTTTCTTTAATTTGCTTATAATACTACTTTTCCCT
TGTAACTGCAACAAATATGTTAGACGCAGAAGCTTCTACTGAAGTGTCATTTCCATTCAGTTCCAGTGTCCCCTCCCTGTCCTCACA
CTCAGGGTGTCTCAGGAGCTCAGGCAGTGGTGGCTGATACGGTGGAGCTTCACCATGAGGCTCTGAGAGGCTCTCCCCCAAGTCCTG
TACCAATTTTACCGCAAAAAAATGTCACCCTGGGGAATATCTCAGCTCCCTCTGGAGAAGGAGTCTCTGTCGGCATCTCTCTGACTG
AAGAACATTCTAGAAACTTCTGTGAGGCTGACAATGACCTTAGGGTACATACTTTTCATCACTGGTAAATATGCTTTAACATACAGT
GTTCACCATGGCTGATCAGGAAAGCGCTTCTCAAAGGCAGGTCTCTTGAGCTATTGGCTCTCTAAATCATTGTGTTTTGGTTCAAGA
GAGCCAGGCTTTAAAGAGTATCTCAGAACTTAGAATTATCAGCTAGCTGCTCCTTCTCTCTGACCACAGTCTATCTTGACCCTTTGC
CTTTCCTTAGGCACAGAGACAAGCTTTCTCATAAACAAATGAGCCATCTTCTGTGCCATGTCTGTCACTACTAAGGCTTGGTACTCC
TGGTCACAAAGGTTCTTCTCCTCCCCAACACTAAAACATCAGATTCAAGACTCAGAGCCACTCAAAACTGCCCATTCCTTCCTGGTT
TTATGCTTAGGTATCCTCTGTCTCCCCCCTCCCCGCCCCCAGACATGACAAAGAACAGAAGTGTTCCTATGGCTGCCGGAATCACTG
TGGGACTGCTCATCATGGCTGTTGGAGTGTTTCTGTTTTATTGCTGGTTCTCTAGAAAAGCAGGTGGGTGACTCCCTAATCACACCC
TTCCTGTCTCTGTTCCTGCTTGCTCACACTGAATATGTGACCATCCGTAGGCTCTCGAGAGTTTCTCCTCTTTCATCGTATGATTCC
TTGCCTTTCCCTCTGGGTCCCTCAGAAGTCCTGTTAACCAAATACAGAGTTGTTCCAAAAACTACCTGAGCGCAACACACCTTGATG
AGACGCCATCTACCTAACCTCAGAAAGGGTCCTGTGTTTATTATATTCCCCCGAGGAAGGAGAAGAAGAGAAAAACACTAAGAAATG
GAAGTCAAAACAGAATTCAAACTAGATTTCGTAGATTACTGATCCTTAAAATGTATAGTATTTTTATAGATGGTCAAGTCTATTTTC
TGGTCTAAGACTATTGAACTATCTGTAGTCATTAAAGACCACTATCTTTGAAAAATCCAGGTTAAAGGAGTATGGACTGAGCTTAAG
GAAGTAAGGACTGAGCTTAAGGGAGGATTGACTGAGGTTAAAAGAGGATGGCCAAAGTTAAGAGAGGATAGACTGAGGTTGAGGGAA
GATGAACTGAGGTTGAGGGAGGATGGACTGAGGTTAAGGGAGGATGGACTGAGGTTAAGGGAGGATGGATTGAGGCTAAGGGAGGAT
GGACTGAGGTTGAGGCAGGATGGACTGAGGTTGAAGGAGGATGGACTGAGGTTGAGGGAGGATGGACTGAGGTTGAGGGAGGATGGA
CTGAGGTTGAGGGAGGATGGACTGAGGTTAAGGGAGGATGGACTGAGGTTAAGGAAGGATGGACTGAGGTTGAGGGAGGATGGACTG
AGGTTAAGGGAGGATGGACTGAGAAGCCTCCCTTCACTTGGGGCCAGCATGAAAAAATCAGTTGCTTTTACCCCTCTCCCCTCTGTC
GGCATAGCAAAGATTCAGATCTCAGTAACAGAAGTTCCTGGCAGTCTCCATGTGAGCCCCACCTAAGCTGCCTCCTAGTCCCCATGA
AAGACCCCTCTTCCTGAAATTCAATGTGTTCCTTTACAGGAGGAAAGCCTACCTCTGATGACTCCAGGTAAGAACTTGGACATATTT
CAGGAAGTAATGTAAACACCCACAGGACAGAAATTTTAAGTCAAATCTCAAATCTTATTACCTTATTATCACCAAGATTACAATTTC
TATGCTCGTTGCTGAGGTGGGTTGTACCCAATTGATGCTACCCCTCCCTTGGGATGCCTCAAGTCAATTCCCAGAACCTTCACCTTA
GCTACACCTTAGAAGCCTCCTTCTCATGTCATGAGGCAATTGATTTTCATTTATTAAATCACTTCCTTGTACTACTGTGAGCTTGGT
TGTCTAAATGATCACTTTGGCAGTTACCAGGGAAACGGGTTCCCTTTGGAGATCCTTATTCTCATTAATAAAAAAATTTAGAAACAA
GCTAAGAAGAAAGCTCAATACAAATTTACTAGAATTTAATTAAAAAACAGGTAGATGAAAATCCTGGTGTTACTAATGAGGAAAGAT
GAAGGAAAAATTCATGCCTTTGAAGTTGGGCGGCATAGGATAGAAATAGTTTATTTAAAAAGGAAGCATCACTCTCAAGAATGGAAC
CAGACTAGCCAGCCAAGAAGAGAGTCCCAAAATCAGCCATCCATGTGGCTCCTGATCGTTATAGCACCACTCTCCCCATTTGTAAGT
CCTGGGCTTCAGATCACACGTGTTCTCTTTGTTAGATGATGCCAAAATGAGGAAGGGCTTCAGGTTTTCCTTCCTCTGCAAACTGGC
TTCCTTCATGTTAATCGTATCGCACAGTCATTGAATCCACCCACACCTTGAGATGACATATCTTAATTAGCCTAGAAATCTCTAGGC
ATGGCCTGCCTGATCAAAATCTGTTGCAATGACAGCCAAGAATGTGGAGCACATATCTCACACCTAGAGGAGTCATTCTGCAAAGAA
GTAGGATCTGTGAGAAGACTGCCACCATGCCACCATCCTGCATCCTGAAGGCTCGCTTTAAGAGTCATCCCACAGCTCACAAAGTGC
CATGAGCCTCCCATAGTGGCCTCATCCAAACCACAGGAGTTCCTTGGGGTCTTTGTTTGTAGAAGATTGTTGTCTCTGAAAGGGGTC
TCAGCCTGTTTTAAATCTTGGCTCTTCCCCTTTTCTAGTTTAGTGGTGATGGTAAGCTACTTCACTTTATAAAACACTCATTGCTTA
TGCATATAAAATGGGAACAAAACAGAACTAAATACTTACAAGTATTGGCATGGTGCCTCTTACATACCATGGGGGCACAACATAGAA
ACAGAACAATGTTGGTTGAGAACTCCTTTGGAGGGATCACTATTCCCTTCCTCTCACTCCGGGAAACATCAGTGTTTCTATCTCTTA
CAGAAACCCTTCAGATTCAGAACCCCAGGAGCCCACCTATTACAACGTACCAGCCTGTATAGAACTGCAGCCAGTGTACAGCAATGG
TGAGGACAAGAGCCCTCTGGTCTCGGGGTAATCACAAGATACATATTGGAGCAGAATCGGGAAGATGATAAAGCCTAGAGGGCTATA
ATCCTTCTAGAGAACAAACTGGCCAAGGGGTAGCTGAGAAGAGAGGGAAGCTCCTTATTCTCCTCTGAAATTCTGCCTTCTTCCTAA
ATAGAGCCTGAGGAAAACGTGATTTACACAGAAGTACGGAGAACTCAACCAAGACAGAAACATGCAGGTAAAAGCTGGAGACCTCTC
CGTCCCTTGCTATTGTTCAGCCTTGGCTTCTCAGCAGGTCTGGGCCAGCCAAGCCAGTCTCTTCACTAGGACGCTAGGATTCCTTAA
CTTGCACCTACATTGTTTGGAGTTAATCCAAAGCCATCATGTAGTGCTGCTGGCTTCTTCATTGTCTATGGATGAACTCACGATAAC
CCGAGTTACAAAACCATGACAGAGTCCCCTCTGAGCATCTGTGTCAGCACCTATAAGGTGGAATAGGGCTATCTATTTCTGGGTGCT
TTTCTAGTTCTGGGTGGCTGAAACAGAACCCTGTAGACCGGGCAGCTGAAATAACAAATATTTGTTACAGATCAGGAGTCTGAAAGC
CCAAGATCAAGGTGCCAGATGGCTGAGAAAAAGTAGGATATGTCTCCTCCAAGGTATGGTTGTGGAGAAGGTTTATTGTGGATATGA
GGGAGAACAGCCAGAGGGATCTGGATGAGTCCAGACTGAACTGGGCCATGGGGTGGCAGGGCAAGTGGACCAAGAGAGACCCAGGAG
CAGGGCAAGCCAGGGGGACTGGTGACCAGTCTCTGCTTTGATGTGTTAAATGGGCACCTTAGTTAGCCATTTGTCCTGAGTTTGAGA
CTTAACACTCTAGCCTTTTGGCTAATGATAAAAGATGATGGAATTTCTTCTATAACTACTTTCTGCTGAAATTGGGGTGTCATCAGG
GCCCAAGAAGACTGGAATATTGATCAAATATTCCAGGAAGGGACAGATGAGGGCAGGTTGGGGAACATCTGTTTCACTTGCTGCCCA
GGTTCTGAGGGACCGCCTGGGGCAAGGAAAGTGCAGGGCTGCTTTGGAGCAATCTGGCATGTGGGTTGCAAGGAACACTTGGGGCTG
GCACATTGCTGCAGTATTGCAGTAGTCTGCAGCTGTTTGGAACCTGCTGGGACAGATATAGACAAGGACAGAAGGTAAATTTAAGGA
GTTTAGGAGACACTTTTTTTTTTCCAAATCAAATATCCTTTATTGAATAAAACAATGCACTATAGAATATTTTCTTTAAACAACAGT
GAAATTTTAGAGTATTCCCATTAGATATACATATACCTTGGAAGATGATAGTTGTTCAATCACCTTTTATAAAAAGAAAAAATAAGA
TTTACATTTTACTTAAACTTGAATTTAGGGTTATTTGACTACCTTAATTATTTTATAGTGACAAAATACCAGGAAAAATTAAAGAAA
TATATTAATGTGATATATATATATATATGTGTGTGTGTGTGTATATATATATATATATATACACATATATATATATATATAATGTAT
TTTCCTCAATTACATTTCCAATGCTATCCCAAAAAGTCCCCCATATCCTCCCCCCCAACTCCCCTACCCACCCACTCCTACTTTTTG
GCCCTGACGTTCCCTTGTACTGGGGCATATAAAGTTTGCATGTCCAATGGGCCTCTCTTTCCAGTGATGGCTTACTAGGCCATCTTT
TGATACATATGCAGATAGAGTCAAGAGCTCCAGGGTACTGGTTAGTTCATAATGTTGTTGCACCTACAGGGCTGCAGATCCCTTTAG
CTCCTTGGTTACTTTCTCTAGCTCCTCCATTTGGGGCCCTGTGATCCATCCAATAGCTGACTGTGAGCATCCACTTATGTGTTTGCT
AGGCCCCGGCCTAGTCTCACAAGAGACAGCTATAACAGGGTCCTTTCAGCAAACGCTTGCTAGTGTATGCAATGGTAGGAGACACTT
TTTATTTTGGGTGTATACTTGAAAGTTCTAGGCCCATGGTCCTGGTAGTTGGGGTAGGGAGCAGCCCCAAAACCAGGGGTAGGCAGT
AGGTGGGAAAACTTAGGTTGCTGATTGACAGGAGTAATTGTCTGGAGTCCATTTGACCTGTGACAGACAGGTGGATACAAGTCATGA
CTTCCTGAAGCTTACAAGAATTTTTTTAAGTTTACAAAAAGATAAGTGTTTTCAACATATTAGCATGACCACTGTTTGCAATCTGTA
CTGAAATCTGCATTGCATTGTAGAAAAAGCAGTGGCCTCAGGCCTTTGAGGCAGAGTAAAAGCTTGGGACCATCAAGGCATGATTCT
GAGTTCAAAGCATCAACACTGCTCCTATATATCCTGAGGCCTGGTTATATAGGTTGGACAGAGGTGGTTTTAGCGTGATTGAGAAGG
TTGTTAAGTCTATGCTTAGAAGACAATTTTGAGCTTGTGACTGTAATAGCAGCAGTGACTTACCAGGGCTAGATTAATAGCTTGTCA
GAACTCCACTGATTAATGTTAAAACTGTGAACTTCAGAAGTCTTTATATGACAACAGCATGGAGAGAGGAAGCGATCTGAGATGGTG
TCATCACTTTAAATCACTGTCTTACACCTTTACAAAATGTGCGCTTACACACCTTAAATTACTACCTTAGACCTTCACAACTCAAGC
TTTCACGTTACCACAACCTGCATTTACAAACTTCCCTACAAATCACTTTCCCAGACCCCCACAACGTATATAAACGTTGGGCTTTTT
CTTTTCAGCTGATCATTTATCATGAGACACAAATGGTTGATTATTGAGAGTAGTCGCTGCAAAGCAAGTTCCTTTAAATAAAGGAGC
TGTACAGTTCATGAAAACCTGTTGTGAGTGTTTCTCTATCAGTGTGGTAAACTATGACTTTGTCTCTGAGCCAGAGACCTAGTAGCT
CTAGAAAAGTGAGTCCTGGTGCCTGCTTCTACATAGAAATGGCAGCTGTAGCCTCAGGGAGAGGAGCTGGGTACCTGCCACTGTTAA
CATACAGCTACAGTTAGCCATCAATATCATCAGAGGTCTGAGAAGGGTAAATTATAGCAGGAAGTACAATCCAAATGTATGTGTGTA
TCAAGTAAAATGGCAGAGAATGACTGCCACCTGGACGTTTGTCCTAGGCTCCTGTGGTCTCCGTGGCTTCACTGAGGGCACCGGTCT
TTGGTTGTCACTCAGTTCAGACAGCATTATCTCTTTCGCTGTTACACAGGAGAGCGTTGGCCTTCGACTACCAGACCCAGAAAGTCT
GAGAGATTCTTCTGTGGAGGAACTGGGAAAAACTGACCCACTTTGGCAAGGCAGAGTAGAGCAGTGAACACAATACTGTACATAATT
TGTCCAGAGCCCTGGTGGTAGGTAAGGCACAGGCAGTTTCAACCAATGGTTAGTGTGACGGCAATCCAGGTAAAGTATTCTGTACCC
ACCTTCTTCTTTTGAGAGCTTAAGGTTGCCTCTAGGAGCTTGCAGTTCTGTCTGCCATGGAAATATTTTTCAATCAAACATGTTAAA
TGCTGTATTAGTGGATTTCTGTAGAAGTTAAGTCCTGGTTCTTTCTCCCAGCCCCATGCTCCCAAAGTAAGACTCAGATTCAAAATA
TATTTGCAGATACCTTGGCCACACAGTTAGGTCTTTAGTTAGATCTCCTCTGACTAAATCATAACTAAAATAACTCAATTACTTAGC
CTGCATTCTACCATGTGGCTGGTTACCTGTGCTCAGGTACCATGAATCTATCTCCTCGCATCTCCCTGGGTAGATCTTCAGAGCCTG
ACTCTATCCCAGAATTCTCTCTCCTCCTGCATGTCTCACCTGCTATTTCCTGCCTAAGCCATAGGCCGTCAAATTTATTATTGGCAG
GTGCCACATCCGTACAGACATAAGATATCTTCTCTACAGATCTCTGAAGACTTTGAGGATCATTATCTGTTGTATATTCATGAGTTA
AACAAAATTGACTTGTTTCTTGCTTTTAGTTACCAGTCTTAGGCTTAACTTGGAAACATATATTGAATACTAAATAAAATTTGCCCC
TGTAAATGAATTACATTTGTACTTAACATGGCTTATGTGCATAGTTTAGAGTACATTAAAGAATTTGATCATTTATAAGGTGGCTGA
CCATTAACTTGCATGTCTTAGTTATCTTTAACACTGTACAAGTTAGTAGTTTTATAATATCAGGATTTTACAACTTTATCTCTGCTA
AGTTTGAGCCTTAAAACTTTGAACTTTGTTGAAGAATTGATTGAAAATCCTTTAGCATTAAAACAAAACTTTAAGCCATAAGTGTAT
TCCATAAAAGGACTAGAAGTTTTACAAAACCATAAGTACAGTTCACATAGAGACCAGAAACCATTTTGGCTGTCGTTTGTTCCTTTT
CTCCTTTCATGATGCACCATTACAAAATATCAGTTTCTTGTAAGACTCTGTTTATCTAAGTAACTAAACTTAGAAAAATCAGTAAAG
ACAAAGAGGCTAGACAATAGGTCTAGTCGGTTAGTTAACCTGTGTAGACCTTAGGAATGTATAAACCTTATTTATCAAGCATATGTT
TTTTATTATTTAATGTTCTAGAAGCCTGATGTTGAGCAGTTAGCAAAGGCATGAGTGGTTAAATATACATCCCTAAAACAGTCCCTG
TTCACCTGAGTAGACTTTATAACCTTAGGAACTTATTACCGCATAAAAATTCATCCTAACCTGAATGTTTAGAAGCCCTGCTGTTGC
CAAGTGTGTTTGAAAGGGAGATACAATGATAAACAGAGGCCTCGGCAGGACTGAAACGTTGTATACTTGTTGCGTTATGCCACATGG
TCACCTTAACCAAGATGGTGGTAAAATTACATCGCATGTACACTTTTTGACCTTGGTAAAGATGAGCTGCCAGACATGTGGTTGCTA
TTTAAAACCATCTATTTTCTAGTAGACCTGTTTTTTAAACAAGAGTTAAATCTAAATTACATATACAAAATATTCTAAACAAGAGTT
GAGTCCCAAATATAGAATGTTGTAATAAGATTATACAGATATAGTTTTTTTTTAACTTTAAGCCCTTCTGTTACAAGTGTTAAGCTA
AATTTTGTTCAGGTTTTTAATCATACCCAATGAACTTACAAACCCTGAGATAGTTTACAGTAAGAGATTGGGGGGAGTGAGGCAGGG
GAGGAGAGAAATTGTAAGGATAAAGGTTGTTTATGATGGAAAGTAGAAAAAACCTTAGAGGTAAGAAACTTTTCGAAGTGTAGAGTA
AAGAAAGTTTAGATATAAGAGATTTGATGTTGTTTGTTTGTTATGCGGTGGTATAAGCTGTTACCATAGGAGAGAATTTAAAATCAA
GCATGCCACACTGACTGAGCAGTACTGAGGCCAAGGCATGCAGAGTAAAACAGCGAGGCTTTAATAAGATCCTTGAGTCTGAGGAGG
AGAAGAGAAAGGGTCCTATGAAGCCCAGGAGGGTTTAAGGAAGCTGAGGAGCTTCCAGGAGGCAGCTGAGAGACAAGGGATGGGATC
TCAGAAGCCCAGGAAGGCTTATGGAAGAAGATGCAGGACCACAGCTCCTTGAGGAAGGTGCAGAAGCTCTGAGAAGGAGCTGGGAGT
AGGTCGCTCCGGAGGAGAAGAGGCAAAGAAAGGGTCACAGGCCCAGGAGGACTTAAGGAAGCTGCAGGACAGCAGCTTCTAGGGAGA
GGCAAGGAAACACCAGGAAAGAGCTGAGAACAAACAGAACAGAAGAGGTTTAAGAATTTCAATCCTGTCTGTCCCCAAGTAAACTGA
GAGACTCCTCTGAGAGAAGTGTCCCAAATCACAGAGACCAGGCATTTTTTTTCCCCATGGGAATGGAGCCCCGTAGGGCAAGAGGAG
CAGCCTGGTGCACTGCTGTGGCTTTTGTAATAATAGTGGACAAGGCAGGCAGCTCCAGGGTGACACTTACCCAGCTGAGGAGGAGAG
AAGAAAGCAGCTGAAAAAGCAGGCTGAGGCAGGCTTTTGGAGAAAGGGAGAGGGTAGATGTCTTTAGTAGAGATATGTGGGTGTAAC
TGACCAAAGAGACAAAATGATATCCACGGATAGGGAGTAGTAGGTGCCAGGAGCCAGAGAGACACTGAGTAGCTATATTTGGGACAC
TTAGAGATAGAAATGATAACATTTACTCTCACTAAATGTGTATCTCATCTTCACCAGGTCTTCCAGGCTCCCTACTGGAACACCTCC
TGGACTTAAAGTCTGCCAAACAACTCACACACTAAGTTTGGCCCCAATACCTCTAAACAGCCACTTAGCAACACTTTTGAAGGAACT
TTTACAACTTTTGCCAGAGATCCGGAAATGGAAGGTTGCCTATGTGCAGGCTTCTCCATACCTTCACTCCAAACCTCTTCATTCCTC
CTGATCCCCAACTCATTTCTATCCTTGATCAGGCTAGCTTGCTTTCTCATTCTTGCTCCCCATTCCTTTGACTCTAAAAATACTCTC
CAGACTTTGTTTCAGACTTAACTCACTCTTGTTCTCGATCCCTGCCTGGTGCCCTACTCTACCACACACTCTCCCGTTTGCTCCAGC
TGGCAGCCTTGAACTTCTTAAACTCTACTTCCTAAAAAATAATTTATCTCTGATTTCTGCTTTTCCATTCTGATCTGCAACAGCTCT
AAGCCAGCTGTGGCCAAAGACAGGCCCGGTGGTTACTAGATCCCTCTGTGCACAAACACAAGAGGGAGTGCTTAAGTGGGTCCATGC
TGAGACATCTTTCCCTGAGGTACCATGATGGGTCTAGTTTAAAATAAACCATTTGGGGGCATGAGAAGGGGTTTGAGAAGAGAGTAG
AGGCAGAGAAAGAGAAAGGACAGAGAAGGACAGAAGGAGGAGAGCGAAAGGGAGAAGAGGCCGGCCAAGAGACAGAGAGGGGGAGGG
GGGAAAGAACAAAAGGAGAAAGAGGCCAGAGAGAAAGGCCAGAGAGAGAAAGGCCAGAGAGATGTGTGTGTGTGTGTCTGTGTGTGT
GTGTCTGTCTGTGTGTGTCTGTGTGTGTGTCTGTGTGTGTGTCTGTGTGTGTGTCTGTGTGTGTGCAAACAGTCCTTTCATAGCAAG
TGAGGATTGTACCTGGCTGTTGCTAGAGAACTGTTGGGCAGAGCCTTGAAGAAATGCTATCATTGACTGTTTTTACAGAGGACCAGG
GCTGGATTCCCAGCATTAATATGGCAGCTAACATCTGTAACTCCAAGATCCAACACTCCTACACAGACATACATCCAGGCAAAACAC
TAGAACAGATTAAAAACAACAACAACACAGATTAAAAATAAATAAATAAACAAATAGACATTATTTAAAAGAAGAGAGCCCATGAAA
TTGAGAGAGAAAGGTGGTGGTGGTGGGAAGTAGAAGTGGGATTGCAGACAGGACCGGACTTGATCCAAATGCATCATATGTATTTAA
CACAACATTAAAAAACAGACTTAAACCATCAAGTAAAGTGAATAAAGAAATGAAAGAAAAAAAACTGGCCATTGTTAGCCCTCAAAG
AAACCTGGGAAAATGGCTAACTGAGACCTATTTCACATATCAAACCCTGCTTCTGTCCACCAGTCTCCCTCAGTCCTTATTCACAAG
AACCTGCTGTAGAATTGTTCTAGGAAGGAACTTCTACATTGCTGTTCATCACCAAAGGAGGTCAGGTCAGGAACTCACAGAAGGCAG
GAACCTGGAGACAGAAGCTGATGCAGAGGCCATGGAGGGATGTTCCTTACTGGCTTACTTCCCCTGGTTTGCTCAGCTTGCTTTCTT
ATAGAACCCAAGACTACCAACCCAGGGATGGCACCACCCACAATGGGCCCTCCCACCCTGATCACTAATTGAGAAAATACCTTACTG
CTGGATCTCATGGAGGCATTTCCTCACCTAAAGCTCCTTTCTCTGAGATAACTCCAGCTTGTGTCAAGTTGACACACAAAACTAGCC
AGTACAATTACTTTAAAGATTTACAAAAGTAAAAAGTTAAGGACCACTACTTTAAAGATAGGTTTAGCTTTGTTTTTGCTACTATGA
TTAAGTTACAATGTAACCTCTTTGTTTGCTAGAACAAATAATTAAAAACAGATACAAATTGTCTAACTCAGATATGCTTTATAGATA
CTAGCTCTCAATCTTGTCAGAGATCTGCTAAATATTAAATGTTTAAGCTTGCTATGACAGACAGAGACTCCCAAATCCTAACAGTGA
CCCCCGCCCCAAGGTCTCCAAGAAGATATGGGCACAGCAGCAAAGGACTCTACCTGGCCTCTATCTCAGAGGTCTGATGACCACTGA
GAAAGACTGCTCCATTGCCTTTCCTGCTGCCAGGGCTCGGCCTGGTCTATGGACAGAGGACACCTGGAGAATTAAATGTCTCATATT
GCCTAAGACAAGATGAGTCATTTCTCCCATGTTCCTCCTCCACAGGAAAAGGCCTCTTATCTTCTGGGCCTAATGGCCAGACTGCCT
CTGCCCAAGTTTCGATGGAGACCACAGAGACCTGGGAGAACTGTTTAGGTAGTAGACTATAGACTAACCTCTGTCATTTTAATTAAT
ATATGATATCTAGACTATAATTAATTCTCCCCAGATTTCTGATTACATTGACAGCTAAACTAACTGCAGCTTGACAGCTAGAAAACA
ACTAACTCCTTACCCCAAGGTGATCCGCCAGCATATTAGCTCATTAGTCAATTTGTTAACTAGTTAAGACTACCAGATCTCCTATCA
AAAAGGTAGACAGGTTTGCCTTGCTCACAGGTTTCATGATAAAAGATACACAAATTTCCCATGTAACCTTTTTGCAGATATAACCTT
CTGCTACTTATCTAAAGAAAATCAGTTTACAGTGACTACTCCCCAGTGATTAGATTTGGTAAATAATTAAATAAAAGGCTTAAATAT
TTATAAAGCTCTAAATGGGGGTCTGGAAAGGTGTTTTGAGGTCAGTAATCATAAGATATAAAGGTTAGAGGACATACAAACTTAGGT
GGTGATGAAAATTGATTCAAAATACAAAGTGCTTCAGATTCCTGCCCCTTGCTATGCCACTGTTAAATTGAGAGTTCAAAGGTTCAA
AATGGAGTTCTGATAAATTAACTTAACTTTAATCAAACATCCCACTATACAGTTGAACTTTTGTAATCGCAGGTTCAAATTTCAAGT
TTCCTCTGATTGTCTTTTAACTATAGGCTTAAGGTGTTTCCCATGTTAACTCTATATGATCAGTTATCATATGCACGAGGTCAAGAT
TTTGAGTTTACTCTGATTGTCTCTTAAATGTAAACTTAAAAATGTTTCTCGTTGTGCTAAATTAATATACATCTACACAGAGGAAAA
TAACTGGCTTGCCCCTTCTACTCCACAAAAGGTTTTTGTCTTCCCTAAGACTGTTTGTGCTATTAAAAAATTATTATAAACTTATAA
CCTCCAGGAAACCCACTCAGATCTACCTCTCATGGACCAAGTAGGCTCTATCTAGATAAGTACACCTGCCAGTCAATAGCCTAAGTT
CCTACCTCTTACCTATTCCAGAGGGTGGGATTTCCCCCCTGTTCCCTGGGTTTAGGACTCCCCTCACCTCAACCTCCAAGGCCTAAG
TGTTTCAAGTGTACACCTAAGAAAAAGTTCTTCTCCCAAACCTACTTCCTACTTAACTTTATTCTGCCTCCAATGTGTGTGTGTGTG
TGTGTGTGTGTGTGTGTGTGTGTGTGTTTCAGCATCTTGCCAAGTCCAGGCATTTACTCAAAATCTACATCCAGAACAGCTCCAGAA
AAGAAACCCGAAGCTTCGTCAGTCTAATCTCACCGATGCTTCTACTGGGCCTGCACTTTCCTACCCACGGATGGCTCCACAGATCAT
GGACAGCAAGGAAATGGCCAACTCTCCTAAGACTGGGCCAACATCCCCATCTTCTCTTTGGTTTCCCAGAGCCACGCCACCCCAAAG
TCAGCAGGAAGTTGCAAAAGATCACAACGACCCTATTCCTGTTTTGTAACCACCCCCAGCCTGAAGCAGGCTGAGCCAGACCTTGAC
CTTGCTGCCACTAAGGAGATTACCTAGGGTGGAGCCTGCCTCTCTAGATCACTCTATTGTTCAGCCACTGCCACTCTTCTCCTTCAA
GACACTGCTACCTGCTGGGAGGCCACTGAGCTATTCCAGAGACTACACCCTATCCTGCACATCATCACCTGTAGCCTGTTCCAGGCT
CCAAGAATGAATTGGCGGCAATGGGCCTCCCCCCTACCCCCTTTATAAGTGCATTTGCCATTAAACATTTGCGCTTTGATCAGAAAC
CTTGACTTAGCTCCATTTTTCTCTCAACCTCCTAGTTCCCCTCTCTTTTCAGCTCCAGCTTGCCTCCCAGGTGAAACCCGGTCCATG
TGAGCCTCGGGTTGGCTTCCAACAGTCTGGCGTCCAAACAGGGACCTGAGGAATGGCAGAAAAATGCTGGAGGAAAGCTGCTTGGTA
TGGAGCTCCACGGGAAGAAAAAGAAAAGGATTGTATCGGGCACCGAAGCAACAGGTACACATGCTAGTGCTAGCTTAAAACTTCATT
TTTTAAAGTGTTGCTAGAGGTGCGGGAGGATATGGGCTAAGTTTGGAATCGGTGCTAACCCAACGCTGGTTTCCACTTAGGTTCAGC
AGCGAATTATTTGGAATTAGGAACTGATCAGGCGATTGTCTGCAGTTTTCAAAAACTGCTTTAGGTGAGAGAAAAAAGGGTTTAAAT
TAAATAATCAGGCAGATGTCCGCAGTCAGGAGCTGCATCAGGCGGCAGGAACAGGGTTTGTAATAATAAAATAATATAGAGGAAAAT
GGATTTTCAAACTCTCCCAGAGGCAGAGAAAAATTGGGGGGATGCCCTGCCCTTTTCTCTCTGGCCCCTACAGCAGAAGTTCTCTGC
CTTCTTTGTGTTGTCTAATATCTAGTCTAATGTCTGGTGCACCGGTCCTTTCACATGTCAATCCATGTGTGTTTGTGTCTAGTTGTC
TGAATGACTGAATGTTCTGTGTTTCATGTTGGAAAATAAAGATGGCTAAAACTTTATCTGCTGGTTCAGCAAAGCCAAGAGTGGCCA
CATGCTTTAGCCAAGGATCAGGTACAGCCGAGAAGCCTCTGCTGGAAAAGCTGCTCTCAAAGAAAGGGAGTCTGCAGCCTCTTAGTT
TGGGGGCAAAAAAGCTGATAGATGTTTTTATTCCTGGAGGGTTCTCTGCAATCGTGATTAATGTGTTTGGCAAATGGCAAAGTGCCT
TTTTTATCTTATAATTATAGCTAGAAAAATTGAAATTCTTTGATTGATCTAAATTCATAAGACTCATGTAGCCACTTCATTTGGTTT
TTTACTGGTCTTAGAGGTGTAATATGCTCTGCGTGTCTTCATCGTAGAGTATTAGCTTATAAGTTATTGGGTATGATCAAAAAGGTG
TAACACTGGTTACTAGAAAGTTAGCTTTAAATTGGTAACTCAGATTTGGAGTCTTTCCAAAACATGGCATAAGGCAGGCCAAGAGGC
TGGGTCTCTAAGGATACTTCTAGTTGAGATAATATTTAATGTGATTCTTATCCTAGAAAGAATTAAAGGTAAGATTTAAAGCAATGT
CTCTTTAATGAAGCATTAAGACTTGCACTGTCATGCATTCATAGGTATAAATTGGAAGCCAAATTTTGTAACGTGATAGTGAAGTTT
TGATGTTGATTCTAAGGTGATAAATTGCTTTGAAATTGGATTTTGCTTTCCCAAAGTTGTAGTTATAGTCTAATATTGCAAAAGAAA
CTTAAAAATTCTAAAATCTGTCCTCAGGGCAAGATGAACAAAGCTCAGACCAGCCTCTGCTGGGCTTGTGTGACTTTCTTTTGTCTT
GCAAACAGTGCCTAAGGAAGCTCTGGAAACTTGCCTCTCCTTGCCTTCAGGGAAAATTCCTTTTAGCTAAAAGAACACTGTTGCAGA
TCTATCCTGATGTTGTTCTAAATAAAGTTCAAATTCAAAGTTTATAAAAAGGTCAATCAGGCTGTAGAATTTATCAGAACATTGCGA
TTTGACTTGCCTACTTCATGTAAAGTTATTATAGATTTAAGAGTTTGTTTTTTCCTTTGCATCTGAAAGAACCTAGTAGGCAAACCC
CCACTCAGCTCCTGATTCAGCGTGCATGCAAGAATCACGAATAGAACACAACACCTTGATGTAACAACACGAGGTATTTTAATGGCG
GAGCTCTGGGTCGAAACGTATCTCACACAGGAGACAGTGGATTCGACCACAAGTCTTGGAAGCTAGGGGTTTTTATAGGAAAGGAGT
GGGGCTGGGGGAGGAATTGGTGCGGTTTCACATGATTGGTCTATTTAAACATCAGCAGCCAGTAACATTTAACTTAGGTCAGAGGGG
TGGGAGATAGGGAGGCGATGGGCCTGCCCGGGCATGTCCTGGTCTGTTCTGCTATGTTCTCAGCCCCAGGTTTCAAAGCTCACAAAC
AACTCTTTGGGCTATTTGACATACATTACATGAATCACAGGTCTCAAGTTTTATTTCCTTTCACATCCAGAAAATTGTAAAGAGTTT
GCTTCTAGTGAGCTTGTAATCATTGGAAAATTTTGCCTCTGGGTACGGCTAATATAACTTTACCTCATGTAAGAGAAATTCTTATTG
TCTTTGCTTCAATACAAGACGCTAAGAGTTTGAATCTTTCAGTATATATTGTTTCCTAAGTGGAAAGTATTTTATTAGCTAATGCTT
CTCAAGGGAAGTTTACTTTAAATCCTTGCTCTCACACAAGGAGCTTTTAAGTTCTGGGGAGTAGCTGTTGCTCCAGAAAAGATTCAG
AGGCAATATCTTTTGAATACTTAGGGTTTTGGTTATACTATAAAATTGTGGTACAGAAAATCTAAGAACAAAAAATTAATTTGCTTC
AAAGTTTTATTTTCAAAAGCTTCCAGGAGATGTTAATTGGCTAAGACCTCACCTTAAACTCACCACAGCAGAACTTAAGCCTTTGTT
AGGTGCTATTAAGTGAGACACAAAATAACTGGCAAAGAACAAAAGGCTTTGCAGAAAATAAAGAAAGCTTTTATAATTGTTATCAAT
TATAATCAATGGTAATTAAATATTAGCTGCCTATTTTAGTTATTGGTCATTAAATGTGCCCACAGCAATTCTTTGGCAAGAGAAAAC
ATTGTAAAGTCAGCTTTCTTCTCCTCCCGGCCGACCATTGGCCCACGTGGAGCCTGCCGGCTGCTGGCTGCTACAGGGCGGGGCCTG
GGGTACACAGCTTTCCCTGAGCAGTGGTGGTGTAGGAGGCCGCCAGAAAGCGGGTGCCTTTGTAGCGAACTCAGGGGAGGGCCTGAA
GCACCGGTTGCACGCGCCATTGCCCCTATGAGCATCCACTTGTTTCCATGGCCGTATTTTTCCTGCTAGGACTCGGGGTGCTCTGCC
GCCCTTGCTCGGGCTTCCTGGCCTACAACAGATTGGGCTGCTTCAGCCTCTATGGCCTGGGAGGAGGGCCGGAGGCAGGCAAGGCCG
AGGTGTCGTTCACCCCAAAGCACAGTTTTAACATCCTATTAAGGCTGCTGTGGTGCTAATAAATAATTGTAAGATAAATTTGTATAT
TTTAGAAAACCTATAACAAAAATTGTGTCTTAAGAACCTGACAAAGTGTTCCAAACAGCAATTAATTTGGTTATTACAGAATATTAA
TATTTGATCTATTGCATACCATCATTTTAAATTAAATTGTCAATCATTATCCCAAGGATAAGTTAAAAAATTGTTTTTATTCATGCT
TCTGTATCTTCTATAAATTCATGCATGCATGCATTCCAATTACTCTATTTAAAAACAGCCCTTCTATGGGAGAAAGGTATATGTGAA
TTGGATCACACGCTTATTCTTTTGAGTTTCCCCCTGCTTCAACACAGATAATTAAATTGTGTGCTCTAGATGGTGTTTTAAAATGTT
GAATAATCAAGCTTTAATTTGTATATTTATCAATATATATCTCAGAGCTTACAATTGCTTAAAATTGTTCATCCCTCAGATGCTATT
AATTCTCAAGTTTGCAATTGCTTATGCAACTCATAAGAAAAAATGCTGTTCTTTTAAGAAGACTGTTTTTGGACAGTTAAGAATTTG
TCCTGGGTTGCCTGGAAAAGACCCCCAGGATTTGCTTTTGTGTTATAGAGATTTACAAAAAGCTTTAGCTGATAAAGGATTACAGAT
AGCTCTTGAAAAGATACAAACTCCGGATCCTTATAATTATTTGGGTTTTAGACTTACTAATCAAGCTGTTTTTCCCCAGAAGATAGT
TATTTGCAGAGACAACTTAAGGACTTTGAATGATTTTCAAAAATTGTTAGGTGATATTAATTGGTTTCACCCCTATTTAAAGCTTAC
TACAGGGGAGTTGAAACCTTTATTTGATATTCTTAAAGGGAGTTCTGATCCTACTCCCCAAGATCTTTAGCCTCAGAAGGATTGCTG
GCCTTGCAACTAGTGGAAAAAGCTATTGAAGAACAATTTTTCACTTATATAGATTACTCTTTGCCTTTACTTCTGTTAATTTTTAAT
ATGATTCATATGCCTACAGGATTGTTATGCCAAAAGTCTCCTCTAATGTGGATACATTCGAGGATTTCTCCTAAACGTAATATCTTG
CCATATTATGAAGCAGTAGCTCAGATGATTATCCTTGGAAGGAAGTAGGCGCTAACTTATTTTGGCAAAGAGCCAGATCTTCTTGTT
CAGCCTTACAGCATAGGTCAAGACACTTGGGTAAAACAGCACAGTACAGATTTGTTGCTTGCTCAAATAGGGTTTAAAGGAACTGTA
GACAGCCACTACCCTCAAGATAAGTTGATAAATTTTTTTAAATGTATATGAGGTGATATTTCCTAATATGACCTCTTTACTACCCTT
ACACAATGCTGTTCTGGTGTTTACTGATGGCTCCTCTAAAGGGCAAGCTGGATATCTTATAAATAATCAACAGGTAATCATAAAGAC
TCCTGGCCTCTCAGCTCAATTAGCAGAATTAACAGCAGTACTGAATGTCTTTCAGTCTGTGAATGAGGCTTTTAATATCTTTACTGA
TAGTTTATATGTTGCACAATCGGTACTCTTATTGGAGACCTGTGGTACGTTTAACTTTAATACGCCAGCAGAATCCCTGTTTTCACA
ATTGCAAAACATCATTCTTGCCCGAAAATGCCCGTTCTATATTGGCCACATATGATCTCATTCTGGTCTTCCTGGACCTTTAGCTGA
AGGCAATGGTTGCATTGACAGAGCTCTAATAGGGAAGTTCTTAAATTCAAATCCTGTTGCTTTGGCCAAAGGTGATCATGAAAAATT
TCATCTCTCTAGCCATACCTTAAGGCTCCAACATAAGATCACCGAGGAGCAGGCAAGAATAATTGTAAAACAATGTCCTAAATATAT
TACATTATCTCCAGTGCCACATTTAGGAGTTAATCCACGAGGTCTTATGCCTAATCATAGTTGGCAAATGGATGTAACTCATTATGC
AGAATTTGGAAATTTTAAATATATACATGTTTGTATTGACACTTGTTCAGGATTTCTTTTTGCTTCTCTACTTACTGGAGAGGCCTC
TAAAAACGTAATTGATCATTGCCTACAAGCCTTTAATGCTATGGGATAGCCTAAATTTATTAAGACAGATAACGAGTCATCTTATTC
TAGCAAAAATTTTACTTCATTTTGTAAAGAATTTGGCATCAAACATAAAACTGGAATTACTTATAACCCCATGGGACAAGGAATAGT
TGAACATGCTCATCACACTTTTAAAAATTGGCTATATAATCTCGATCTTTATATATTCAAAACAGTAATGGTTTAAGATAATATTTT
GATATTTTTGGAGAATGTGTGTGTCAAATTGTAAAAAATTTGCTCTTGGTGTCCTTAGTTCCTACCTGTTTCCACATAATGGTGTTA
ATTCTTGAGGACCTATACTGAATAAATTGCTGCAAATGAATGCTCTTATTTCTGATTAATAAATAAATTATGCACATGTGACTATTA
ATAACTTTTCAAGCTTTCTAGTTGCAACCGCTCTTCAAGAGAAACAATTGAAAGTGTAATTAGTCACTCCCCTTGTTACATTAAAAC
TGACTTTAACAGTCAAGTATTTGAGATGTTTTGTCAACAATTTAATAATTCTCAAAGACAGGGTATTGTGGAACTTTAAAAACTCTT
CTTCTTAAAAAACAAAGAAAGGGGGAAATTATATCCCTGTGCACATTATTTAATATATCCCCATGCACATTATTTAAATCATACTTT
TATTTTAAGAATTTTGAAATTTGGATGTCAAAGAACTTAATAAATGCCTCATAGTATCTTAAAACCAGACATAATCATGTCTAGGTG
AGATGAAAAGGATCTACTTATTGGCATATGGCTTGATCCTGAACAGCTACCTTATTGGGAAAGGAAAAAGCATAGGTTCTCTTCACA
GAACGCTGCAGAAGCATGACGGCTAATTCATGTGACAAGCTGACAGGTGAGTTCCCTGAGTGCCCTGACTGTAGTGACAAGAAAGCT
GAATTGGGTATATGTTTTTGACCCACCTTTGCTTGCCATTTTCCCAGTGTGGACAGGCTAAGCTGCCTTGCTTCAAGCTGTTTAAAC
CTTATGGGACATAGAACATTAGAGACTGTGTAGTCTGACTTAGAAAGATTAGTCACAAGAAAGAGTTATAGTGATTCTTCTCCTTCC
CTCATTGTGACCAGTTATTCTAACTCAATCTTGAACTGGTCTAGTAATAATTCCAGTATTAGAGAATCCTAGGGAAGATAGCTAAAA
TCTTAATTACCTAGCAAAGTTAATTTGGAGCACAGCCTCCACTCTCAGAGAAGCTGCAACCTTTCTGCAATTCTCAAAGCCACCTCC
CCTACACCAGGAGCTCATAGTCTAAGCTTGATCGTTGCTAATTTCCAATTGAATGGAGTACCTGGACTTCCCCAAAGAAGCTTTTGT
ACTGTTGCTTTTCACTGTCTCAACTTTGTTTTTCAAGCAGGTCAGCCAATGCCCTTCAGCCTGACTGCAGCAGGTGTGCAGCCTTGC
CTCCAACAGCGCAGGTGGCAGTTATTAATCTTCATGAAGAACCATTCTAAGTGCATATTGTGGCTTTGGACTGAATTGGGAATAAGG
TGTGCTAATGAGTGCCAGACAGTCAAAGGTGGGCACCCACATCTTGGAACCATACCAATCTAAGACAGAGGTACATGCCAAAAGGCC
GTGGTTGTTGATAACACACCACAGAGCCATGTTTTGTGTGAAATATAACACAAACAGCTGCATGTAGCCTCCAAGATGGAATTGGAT
GGTTCGGGAAAATCGAAGATTTCCTAAGACAGGCGCAGCCGCCAGCCACCATAAGTCCCAGGCAGGACTAAAGAGCATAAATAAATT
TTATGCCTCAGTCCAGCTTTTTTTTAATATTAATAACGGGGGGGGGGGGATTGTAACTACCCCCAGCCTGAAGCAGGCTGAGCCAGA
CCTTGACCTTGCTGCCACTAAGGAGATTACCTAGGGTGGAGCCTTCCTCCCTAGATCACTCTATTGTTCAACCTCTGCCACTGTTCT
CCTTCAAGACACTGCTACCTGCTGGCAGGCCACTGAGCTATTCTGGAGACTACACCCTATCCTGCACGTCACCTGTAGCCTGTTCCA
GGCTCCAAGAATGAATTGGTGGCAATGGGCCTCCCCCTACCCCCTTTATAAGCGCATTTGACATTAAACATTTGGGCTTTGATCAGG
AACCTTGACTTAGCTCCACTTTTCTTTCGACCACCTAGTTCCCCTCTCTTTTCAGCCCCGGCTTGCGTCCATGTTAGCTGCGGGTCG
GCTTTCAACACTTTCACCATCACTGCCGCAGACCCTCTGGGTCCCTTTGTCTGTGTGGAACGAGTCTCTGGGCAGGTGGACAAGGCG
TCAGCGGGTGACAGACAGACCAACACGGGAGATTGTGTAGAATCTGAATGTATTCTCACAAAGTGAACACCAGTCTTATATAGTACA
GAAAATAAAAAAAGGTAGCAGGCAAAGAACATTGAAGTTACCTGATACAAAACAAAGGAATGACTTCAAAAGGATTTAGAGGAACCA
GGTAGATGTTTACAGTAAAGATAAAGCAGTCCTGCCTAGGGTCAGCTTAGTGACAGGTAAGGATTTCACACTCTAGTGCTATACCTT
TGAACCTTGTGAAAGCTAGCACCAGGGGGTTCTTCTCTTAGCAGGCCTCATGAATAATGCAACATCACAAACCCCTCCCCTCCCCAT
TTCCTAGGCCTTGCCAAACTCCCACATGAGTGTAACTTTTAAGTAACTGGAGAATCTCCATTTGTCAGGAGAATTCACCAACTTGCT
TCTAATATGCAATGCAGCCTATACTGAAGATTTCTATCTCAGTGGGATTCCCTGGCTCTATTATGGTATTCCCTTGTTTGGGTGAGA
TTTGCTACTGTCCTTAGTAAATATTTTGAAGACCAGCTCTTAGCTACTGGCTCCGTCCTTTGTAATGCTCAATTAGTAACTGAATAG
GTTAACTTCCTTACTGCATTCTTACAGGATTCTAAGCTCAGCTTCTGGGACCTGGAAACACTAGGGAGACTTAACTATATATAACAG
AATTTAATCTTAAAAGGCATTTATAATAAAATATGAAAAGAGAGCTCATAGTTCCATACACCAGACTAACGCGGGAATAGAGTATGA
GTATATGGGTAAATGAGAACGCCAAAGCTCCAGGAGGTGAGTTTCCGTAAAACTCTTTTCCTCGTTGAGTGCTTACAGACTTTCAGC
CTGTCAAACAGACTTGACCGGAGGGTGTGGCATATCGCCTCTTTCTAGTTTCTTAATTTTTTAAATTAAACCAAAATGGAGGAATGT
TAGCATTTCTTCTAAGCTCCACCCAACAGTTACCTAGGAACAGCCAGGTACCAGTCTGGCTTGCTATAAAAAGACTGTTTCTCTTTC
TCTCTCTCTCTCTCTCTCTCTCTCTCTCTCTCTCTCTCTCTCTCTCTCTCTCTCTCTCTGAATGTTTCCAGGATCAAATTCTGAAGT
TTCCAGCAACCATAACAGTATTACACACTGAACAGTTTTTATGCATTGGGTTCATTTCTAAGGCCTGTAAGTAAAAACATATTTTCT
CCCTTAGCACTACACCTGTGTATATGACTGCAGCATATATGAAGACAATGAAAGGGAACAAAGATACTGTCCACACTAGGAAGATAA
CAGGTATTATGCAGATACAGATATATACACAGCACACACATACCTGCATGTAGACACACGTGTCTTCACAGTAATTCTATATGCAGA
TAGTGAGATGGCTCCCATCATACAGATGAGAAACAGAGGCCAGGGAAGCTGAGTGCTGTGGCCCTGGCATAGTGGTGGCAAGGCATA
CCCAGGGCAAGGCATGGTACCTCTGCCTCCCTACAATGTGTGGCAATAATCTAAGATCAAACAACTAACAGAATAGAAAGCAGCATC
CTTCCCTTGAGTGCCCTCAGCAGCATGGCAAGCTGCTGTTGGGTAGGCTGGTCAGGGAAAGGATGGAAGGCAGAGGGTGACCTCACT
ATACAGGACACAGAGTCCTCTAAGCAGCACCCACATCTTAATTCTCCATTTCTTTGTACAGTGGCCTCTGAACTCAGGGTCCCTACA
GATACAGTGAGTGTTCTGATGCCACAGGCGTCCCTTGTGGCCCTGCCAGCTCTGCCCTTACCCTGATGCTTCATCTTCCAGGTATCT
TGTCATTTACTCCCATGAAGCTGACAAGAATCACAGACTGTAGGCTCCAGAAGTCGGTTACCTCAGCACTCCATAGATGAATCCACA
GACCTCCCACCTCCTGCCTCAGCTGGTACACCCTTAGCATTCTCCTCAGGAGAGAGAGTGCTCTTTGGTGAGGCTTCGGTGCATCCT
CCACTCTCTGTACCAAGTGGCCAGGTCTCAGGGCCCTTCTGAGCTCCCACCAACCCCCAGGGATAGTCATATCTCCTGGCTTCCTCA
GTGACCTTCAGTTTTGATGGCTGTTGCTCTCCTAGATTAGGACCATTCAACTGTATCTGGAAGTCCTGTTCTAGGGGAACATGGTGT
CAGCCGGGTGGCCCTCTGTACTGCTCCACCAAATGGGAACACACACTCTGGACCTCGGACTCATGAGGAACTCCTTATGGCTCGGCT
GCATTTACTCTCTTCTGATTGTCTAAAAGTTAAGGTCAGCACCTGGTGTGAAAGTCAGGATCAGCAGCACCTAGGACTCCATAGTCC
ATGTTGAGCTGTAGAAAGTACTGTGCAAATGTAGGTAAACCATGAGGAAGAGAGCAATATGGAATCCTATACACAAGAAACCCAGAA
GAACCCTGCTCTTTCCCAAGTGAAATGTGAGAACATCTGGGCAGGAAAGACTGTCTGCCCAGAGTTAGGGCTTCAACTTTCCATGCG
CTCGTGATCACCCAAGGACGTGGTGGACATGCAGGTGCTCATCCCAAGTCTGGGTAGAGCCTGAGATTCTGCATCTCTAATAAGCTT
CCAGATGGTACTGGGATCTAATAAGCGTCCTTGTTGATTTTCTCAAAGAACCAGCTCCAGGTTTTGTTGATTCTTTGTATAGTTCTT
TTGTTTCTACTTGATTGATTTCAGCCCAGAGTTTGAATATTTCCTGCCACCTCTCCTCTTGGGTTTATTTGCTTCTTTTTGTTCTAG
AGCTTTCAAGCGTGCTGCTCCAGTTTCTTTTTGGAGGCACTCAGCTATGAGTTTTCCACTAAGCACTGCTTTCATTGTGTCCCATAA
ATTTGGGTACATTGTTCCTTCATTTTCATTAAATTCTAAAAAGTCTTTAATTTCTTCCTTTATTTCTTCCTTGACCAAGTTATCATT
GAGTGGGGCATTGTTCAGCTTCCATGTGTGTGTGGGCTTTCTGTTGTTTTTGTTGTTATTGATGACCAGCCTAAGTCTGTGGTGATC
TGATAGAATGCATGGGATTATTTCAATCTTCTTGTATCTTTTGAGGACTGTTTTGTGACCAATTATATGGTTAATTTTGGAGATGGT
ACCATGAGGCGCTGAGAAGAAAGTATATTCTTTTGTTTTAGGATAAAATGTTCTATAGATATCTGTTAAATCCCTTTGGTTCATAAC
TTCTGTTACTTTCACTGTGTCTCTGTTTAGTTTGTTCCCATGATCTGTCCATTGATGAGAGTAGGGTGTTGAAGTCTCCCACTATTA
TTGTGTGAGGTGCAATATGTCCTTTGAGCTTTAGTCAAGTTTCTTTTATGAATGTGAGTGCCCTTGCATTTGGAGCATAGATGTTCA
GAATTGAGAGTTCATCTTGGTAGATTTTTCCTTTGACCAGTAGGAAGTGTCCTTTCTTATCATTTTTTTTGATAACTTTTGGTTGAG
AGTTGATTTTATTCGATATTAGAATGGCTACTCCAGCTTGTTTCTTGGGACTATTTGCTTGGAAAAATGTTTTCCAACATTTTACTC
TGAGGTAGTGTCTGCCTTTTTCACTGAGGTGTGTTTCCTGTATGCAGCAAAGTGTTGGGTCCTGTTTATATATCCAGTCTGTTAGTC
TTTGTCTTTTGGGGGAAATTGAGTCCATTGATGTTAAGAGATATTAAGGAAAATAAGATTGTTACTTCCTGTTAATTTTATCGTTAG
AAGTGGAATTATGTTTATGTGACTATCTTCTTTTGGGTTTGTTGAAAGATTACTTTCTTGCTTTTTCTGTGGTGTAGTTTCCCTCCT
TGCGTTGGTGTTTTCCATCTATTATCCTTTGCAGGGCTGGATTTATGGAAAGATTATGTAAATTTGGTTTTGTCATGGAATATCTTG
GTTCCTCTGTCTATGGTAATTGAGAGTTTTGCTGGGTATAGTAGCCTGGACTGGCATTTGTGTTCTCTTAGGGTCTGTATGAGATCT
GCCCAGGATCTTCTACCTTTCATAGTCTCTGGTGAGAAGTCTGATGTAATTCTGATAGTTCTGCCTTTATTTGTTACTTGACCCTTT
TCCCTTACTCCTTTTAATCTTTCTTTGTTTAGTACATTTGGTATTTTGATTATTATATGACAGGAGGAATTTCTTTTCTGGTCCAGT
CTCTCTGGGGTTCTGTAGGGTTCTTGTATGCTCATCAGCATCTCTTTCTTTAGGTTAGGGAAGTTTTCTTCTATAATTTTGTTGAAG
ATATTTACTGGTCCTTTAAGTTGGGAATCTTCACTCTCTTCTATACTTATTATCCTTAGGTTTGGTCTTCTCTTTGTGTCCTGGATG
TTTGGGGTTAGGAGATTTTTGCTTTTTGCATTTTCTTTGACTGTTGTGACAATGTTTTCTAAGGTATCTTCTGTACCTGATATTCTC
TCTTCTATCTCTTGTATTCTGTTGATGATGCTTGCATGTATGACTCCTGATCTCTTTCATAGGTTTTCTAACTGCAGGATTGTCTCC
TTTTGTGATTTCTTTATTGTTTATATTTCTATTTTTTAGGTCTTGGATGGTTTGGTTCATTACTTTCGCTGGTTTGATTGTGTTTTC
CTATAATTCTTTAAGGGATTTTTCTGTCTCCTCTTTAAGGGCTTCTAGCTGTTTACCTGTGTTCTCCAGTATTTCTTTAAGGGAGTT
ATTTATGTCCTTCTTAAAGTCCTCTATCACCATCATGAGAAGTGATTTTTAGATCTGATCTTGCTTTTCTGGTGTGCCAGGACTTGC
TATAGTGGGAGAATTGGGATCTGATGATGCCAAGTAACCTTGGTTTCTGTTGCTTATGTTCTTACGCTTGCCTCGCACCATCTGATT
ATCTCTAGTGCTACCCTCCCTATCTGACTGGAGCCTGTCCTTCCTGTGATCCTGGGTGTGTCAGAGCTCCTGGGAGTCAAGCTGTCT
CTGGGACCCTGAGATCCTGGTGTGACCAAGCTCCTGGATCCTGGCATCCTGAGATCCTGTAGTCCTGAGCGTGTGAGTGCCTGGGAG
TGGATCTTCCTTTGGGTGTTGTGGAACTGGCTGTGGATTTTGCGCCCAAGGTCTGCTCAGGGCACTGGCCCAGATCTGAAGGTGAAT
TTTTGTTTCTTAACCCGTTCTGTCTGTCTGTGTTTTTTGATTGGAAAATTGAGGCCGTTGATATTAAAATTATTTATTTAAATATGT
GTGTTGATTATGGCCATTATGTTATTTGAGTTCTTGATGGTAGTTTGTTGGTTTTGTTTGTTTGTTTGTTTGTTTGTTTGTTTTAAT
TATGGGGCTTCATATATCTTTTCACTGCCTCTTAGCTTTGTTCATTCTTCTCATCCAAAAACAATGCTGTCTGTATTTTCCTTAGGT
TTGTTCTGTTGGATATGTTTTTTAGGCTATTTATATCACGGAAATTTTTCTTTCTCCTTCAATCATGGAAGATAGTTTTTCTGGGTG
TAGTATTCTAGGTTAGCATCATAGTCTTTTAGGACTTGGAATGCATTGCTCCAGCTTACTCTGATCTGTTTTCCTTTAAAATGACTT
ACATTTTTTTTCTTTTACTGCTTTCAGTACACTTTCTTTATTATATATATTTACTGTTCTAACTATGATATGCTTTTGGGAATTTTT
TTGTTCTGTCTATTTAGTGTTCCATGTGCTTCTTGTATTTGTATGGGTGTATTTCTTTCCACATTTGGGGAAGTTTTTCTTCTATGA
TCTTGTTGAAGATCTGGCCTATGCCATCAACTTGGGATTTTTCTCATCTATGTCTATAATTTGAAGGTTTGGCTTTTTCATGGTGTC
CTACATTTTCTTTGCGATATTTTCCTGTTCTGTTTTGTTTTGTTACCTTTTTATGTTTCTTGTTCATTTGGTCTAGATCCTCTACTT
TATTTTGGAGAACTAATATTCTATCTTCTGCTTGATTCATTTGACTTGTAAAGTTCTACTGTGAATTTCTACAGTAGAACTTTAGCT
ATTATCTTTTTCTTTTTTTATTTAATATTTTTTATTATTACATATTTTCCTCAATTACATTTAGAATGCTATCCCAAAAGTCCCCCA
TACCCTGCCCCCCACTTCCCTACCCACCCATTCCCATTTTTTGGCCCTGGTGTTCCCCTGTACTGGGGCATATAAAGTTTGCATGTC
CAATGGGCCTCTCTTTCCAGTGGTGGCTGACTAAGCCATCTTTTGATACATATGCAGCTAGAGTCAAGAGCTCCGGGGTACTGGTTA
GTTCATAATGTTGTTGCACCTACAGGGCTGCAGATCTCTTTAGCTCCTTGGATACTTTCTCTAGCTCCTCCATTGGGGGCCCTGTGC
TCCATCCAATAGCTGACTGTGAGCATCCACTTATGTGTTTGCTAGGCCCCGGCATAGTCTCACAAGAGACAGCTATATCAGGGTCCT
TTCAGCCAACGCTTGCTAGTGTATGCAATGGTGTCATCGTTTGGAGGCTAATTATGGGATGGATCCCTGGATATGGCAGTCTCTAGA
TGGTCCATCCTTTTGTCTCAGCTCCAAACTTTGTCTCTGTAACTCCTTCCATGGGTGATTGTTTCCAATTCTAAGAAGGGGCAAAGT
GTCCACACTTTGGTCTTCATTCTTCTTCAGTTTCATGTGTTTTGCAAATTGTATCTTTTTCAATTCTATCTTCATTTCAGCTTCAGT
CTTCCTCAATATTTCTATCTCCTGATTGAATTCTGTTCTCAAGCCTTGGATTATCTTTATCATTTCCATCAGCCTTATGCTTTCTTG
AAACCACCAGTTTGTGGTTTCTTGGACATCAGTCAAACAATTATTTTTCTTAAGCTCTTTGTTCTTGATTTCAGCAAGCTGGTTCTT
TGTTTTATCTTTAAACTCCTTGTGCTCTTTGAGGAAGTTTATAATGGTTCTTTCAAATTCTGTGTTCTGAGGTTCATCTAGGTAATT
CTCATTGTTAAACATTTCTACAAAACGGGTACATTTTAGAGAGAAGATGGTGGTTTGATTCTTCATACTGTTATTATTGCAATGAGT
AGAGCATGTGAACTTCCTTTGTTAGTTCAAGTTTCCTATGGACAGAAAATGTTGGGGTGGAAGGGAAGATGTGGGTGCAGGTTGGAT
CTAGATGTTAGAAATGGCTTAGTGGAATTGAAGTCAGGTAGACAGAGGGAACTGGGATGGAATAGAAGATGTGTTTCCTGTTCCAAT
CATGGAGTGATGGGTAGAAAGGTTAGATATGGCATGGTAGAGTCCTATAGTTTGGGGATATGTAGTGAGAGGATCCTGCCTAGGGAT
CTTTTACAGTGAAGGCAGGGTATGTTGGTGGGAAGGTTAAGAGATCAAGGCGAAATCCTCTGGACTGTGGAAAGGGTATAAATGGGA
GGTCAGGGATACACACTAGGCTAGACCCTTGAGGAAAACATGGGAAATCTGGATATTTGGGGAAGCCTCATCTTCACAAAGATGGGA
TATCTTTTGAGGTTGTAGAGTTGGCCCAACAAAGAACTGGGGACAGGGGCTTTGGGTAGGGCAGATTTCAGCAGAAACCTAGAGCTT
GGCTGTACTATAGACAGTTTAGTCAGAGTAGGCCAAGGCACAGAATGTGAGACCCAGGCTAGAGCCTGGAGGAGGACATGGAAAGTC
TGGGTGCCTAATGGGGTAGTGTGGAAAAGACTTAGAGTGGATCTATTCAGTTTCAGGTAGCACAAAATTTGATAGAAGCCTAGATGT
TAGCTACAGTGCAAGCAAGAGTCTCTAGTCTTCCATTTTCTTTTCCTTCCAAGCCCAATGCCCTGAAGACAACATTGTATGTAACTA
TAGTTTAAGACCAGAGGCTGGCCCCTGTTCAATGTACCAGGTTAAGCCACTTACCAAAGAAAGCAAAGAAAAGTAAGATGAGAGGCA
AGAGAATATTTTATCAAAACAGTTGTTTAGAGAGGCACAAAACACTGGAGCCCACCTTCATGGTCCTGGCATGAGTTTTAACATTAA
ATAGAGGAAGAATTGAGGCAGTGATGAGAAGCACATATAAATAAGCCATCTTGGTCCAAGAGTGTTCCAGTTACTCATCCTCTACAG
TCTGATTCAGCCATGTGGCCCAAAGAAGTCCTTCTTGCTAAGGGAGCAGTCTTGTTCCCCATGAGATGGTTGCTCCTACTTAGGCTA
TCTCATGATGGAATGATTTGTTTGTAGGTTCTGATGTGTCTGGAATACAAGATGAACACAGATTTAGAATCTTGATGAGGGTGTGGC
AGCATTCTTTGATTACACACATCTTTGTTTCTTTAGCCTTGAAGAGAACTGAGAGTATTATCATTTTTGAGACAGTTTTAAGCCTGC
CTTCTGACTGGTGATTCTCCAGCCTCAGCCTCCCATGTCTAGAATTATAGCAATGCATCACACGGCTGCTCAGACAGATGTTCCTTC
GGTAATTAGCATGCCCAAGTTAACTGTCCAGTTAAGCATAAAGTTTGAAGGAACGGGGTGGGGGGTGGGGAACAATGAAAACTGAAG
TCCCAAAAGCTAGACTTTCACTCAGCTCTGAGTCACCTTACAGGCCCCAGAGAGGAGTTGGTGCTTTTCATTAAGAGCAATGTCTAA
GAGCCTGTTATAGATTCCCTTTAATATATTTCACCTGCTAAAGAGCATCCAGAAATGGAATGTTCTCCATTCTGTCACAAAAGCTCC
CTATTTTAAATGACGCTGAAGAGCTCATTGATCATTCCACAGCCCTATTTCACAAACGTGCTCTATCCAGTCCACTAAGCTGCATGA
AACTCACTGGATGTTCATTGCTGGCTGGGGTTCTTTGTAATACGACAGTCAAAGCCTTCCTACCATTCAGCTCATGAGCTCTTACTA
TTTTGTTTCTTTTCTTTTTGAAAACTTCATACAAGAATACATTCTTGTGTGTTGAGCATTCATTCCCCCACCCCTACCCTCTGACTC
AGTGCTTCTTTCTCACCAGTCCCCTGATAGTTTCATTTCTCCTTTCACAACACACACACAGAGACACAGACACACACATACACACAT
TGTGGTTCAGTGACAGAATGCTAAACGAAACCAACCACATCATATTTGCATAGGATCTTTTAGTTCTAGAAACTGTTTGTTCTTTTA
TAAAAGAGGGTTTTCATTGAGTTTCCTGAATCTTGAGGCTGAGAGTCTTTTCACTGGGAGCTGGAAACTCCTCACATACAAGAGTCA
CATTTGTTCCTTTGAGCCTGTGTCTTACTCCACAATACATACTCTCCTGAACATTCCAAATTGGTGGTCACCTCCTCTGGTTTTTAG
GGTTTGCTAAGAACCATTATGAAATAATTCCATTTCTTCTAGGCTCTGCCCCACAGTTACCAGGCAACAGCCACGTATGCCTGACTC
ATTATAAAAGGGGCTACTTGCCCCCTCCTCACTCTCTTGCTCTCTTATCCTCTTCCCTCTCTGTCCCTTCTCTCCCCATTCCCCTCC
CCCATCTCTCCACATGTTCATGGCTGGCTTCTTCTCTTCTACTCCTCTATTCGCTCTCTCTCTCTCTCTCTCCCTCCCTCCCTCCCT
TCTCAACTCTCAACTCTCTCCTCTCTCTCAACTCCCCTCCCCATGCCCTAAATAAACTCTATACTATATCCATCGTGTGACTGGTCT
CTCAAGGTGACGGGATGCCTCAGCATGGGCCCACAGAGGCACCCCCTTCCCAGCACCATACTGTGCCTCTACCAAACATATCCCTGA
CTTCTTTTTCTTTACTTTACTTTTTTTTTTTTTTTTTTATGAAACACAACACCCAGGGCCAGGCAGCGTGTCAGAGAGGAGGAAGAA
TCAATACCCAGGAATGATAAGGAGGTTTTCTGCACACTGTTACTCTCATCCAACTTTTTCATATTTTATAGGAGAACAGGTGATAAA
ATGTCATACATAGATTTAAGACATGCCCAGGCCCTGAAAAGGCTTCTGCTTAGTGGCAATGAACCTAGAAAGGAGCCCCTGAGCCAG
AAGAGAAGTGTACACCAACAACTCTCCTTCCTCACTGGAGAAGCCCAGGCCAGTGCAGCCCTCTCCAGCTCAGACTCCAGAATCTAG
CTGACCCCTCTTTCACAGCACAGCTTCAAGACACTGTGACCTCCTGTGTTTCAGGCATGGATACCACAGTGTGAAATCCTCACTTCT
CTGCTCCAGGACAAGGTCTGACATCTGTTTCCATCCCTTTCCCCTTCAGCCTCAGTCTTTCCCTCTTGAGAAGACATAGGGCCTCAC
CCCTTTTGTAGCCTAGAGTGCCTGAGCTTGGATCCTCAGCCTCCTCTTCACTGTAAGCTAGTTGCCTCTCTCCCTTCATCCTGATTT
TGTAAATGTTAACAAATATTAAATGCTCTGAAGAAAGGCTAGAGAGATGGCTCAGTGGTTAAGGGCACTGACTGCTCTTCTAGAGGT
CCCGAGTTCAATTCCCAGCAACCACATGGTGGCTCACAACCATCTGCTTTGTAATCTGATGCCCCCTTCTGGCCTGCAGGCATGCAT
GCAGATAGAGGGCTAATAGACATAAAAATAATATCGAGGGATATTAGATCTTGTCACAAGCCTTTTCTGCATCTACCAAGTTGACCA
TGTGATTTATGTCCATGAGTCTATTTAATTTATAATATTTATTGATTTATTATATTCTGAACATCCCTGAATTTCTGGGATAAACTC
AACTTCATCGTGGTAATTGCCTTTCTGATGTATTCTTAAACTTGAGTTGCAAGTATTTTACTAATCTATTATTGTCTAATATTGTCA
GGGAAACTGGTCTGTCTGTAATTTTATTTTGGTGAATCTTTATCTTTTGATAGCAGAGTAATACCAACTTCATTATAAGAGTTTAAT
AATGTTTCTTCCTTTCCTAATTCCCTCTTTAGTTGGGAAATTTTTAATTTCTGTTTATTGTAGGTTATTTTCATTTGATTATAGGAA
TTTTTTCATTTCCTTCTTGATTTTTTTCATGGACCCATTTGTCATTCAATACTGCATTGCTTAATCCCCTGAGTTTGTACATGCTCC
AGGGTTTCTCTTGTTGTGGATTTGTAGCCTTATTCCCTAAATAGGATATTAAATATTAGAGCTCTTATATCCAGGCTATTCCCACCC
AAGAGAAGGAGTCCAGGAAGAGTTAGTTCTTCGTGAATTTGAAATTTTTATTGAGCCACATAGACCTGGCAGGGGACACATACTCTT
ATAACTACAAATCCCGAGGCAGTCTCAAGCTGAGTGAGCACAGGGTTTATAAAGGTAAAAAATAAAATAAAATAAAAATAAAATAAA
AACACAAGTTTGTTATCTCCCATTGACATAGCTATAATGGCTGTTAGCAAGCATGACAGGATGTGGACCTTTAATAAAAGCAGGGCA
CAGCTGTGTCTCCATAGTATTTCTGTTTCAGAGATGAGCAGAAAGCCTTATAGTTACAGAAGCAGAACAATGCAGGTTAGCAGAAGT
TCCTGTCAGTACCATACATGCCTGAGAGTCAGGCAGACCTCAGCTTTAACAATTACATCTCTCTCACCTCAGAGCAACCACGGCTGT
TTGATATTACCACTATGGTGCTGTACTACTACCCAATAATAACTATGGCCCCTTTGGCTGCAATAACTGTTGGGAGATGAACAACCA
AGGATAGTATGGAGTGAGACCATATCTTAGTTACTAAGATTAAGAGTTGGAGGAAGCTGGGCAGTGGTGGTGCACGCCTTTAATCTC
AGCTCTTAGGAGGCAGAGGCAGGCGGATTTCTGAGTTCAAGGCCAGCCTGGTCTACAGAGTGAGTTCCAGGACAGCCAGGGCTACAC
AGAGAAACCCTGTCTCGAAAAAACAAAAACAAAAACAAAAACAAAAACAAAAAACAAAAAAAAGGAAGAAACTCATTCCTTCATTTA
AAAAAAAAGAGTTGTAGAAAAATAAAAATTAAATATTAGACTGACATTGAAATGAGAAAAAAAGTGGTGGCTAATGTATCTAGGAGG
AGGAAGTCGAGATATTGTTAGTTAGGGTCCACAGATTGTAGAGACTGTTAGCACTATGGAAGGTTATAATTAAGAAATAAATAGCTA
AGGGGATAATAATGAGCAAAAGAGAAGATGTGGGAATAAAAGGAGTAATAGCTGGGGCTGGAGAGATGGCTCAGTGGTTAAGAGCAC
TGACTGTTCTTCTAGAGGTCCTGAGTTCAATCCCCAGCAACCACATGGTGGCTCACAACCATCTGTAATGAGATCTGATGCCCTCTT
CTGGTGTGTCCAAGTATAGCGACAGTGTACTCATATACGTAAAATAAATAAATCTTTTTAAAAGGGGGGCAGGGGTAATAGTTAAAC
TGAAGGATAAATAATGCCTTAGAGTAAATCATGCAGAAAACCACAAGCAACAGCTCTGACTGTAAGCTGAGCAATGTTCAACAGAGG
TAAAACCATCATGAGACCTGTCTGAGAAAGTGGAATTTAAGTATCAGGAAATAATCAATTAAAGACTATGTATGGGGCTAACTGTGG
GCACAGCTATGCGGACGGGAAGGGTAACGTGTCCAGACGCGTCTGGTTCTAGCAGTTGCTATGCCGTGCTGGACGGTGGGGAGTCAG
GTGAGGACCTGCAGTGTCTGAAGCTTCAGCGTCCTGGGGGTCTCTTCTGGGTCTTTTGTGCATCCACATTAATTTTAGAAATTTTCA
TCTAGGTCTATGAAGATATTATTGGAATATTTAGGAAGACTATATTAAATGTATGGGTGCTTTAAGTTGCATGGCTATTTTTTTTTT
ATTATTAATCCTGCCAATCTGAGAATGTGGGAAGTCTTTCCGTCATCTATCATCTTCATGTTCTTTGGTGCCTTACAATTTTCACTG
AGGAAACCTCTCACCTCCTCGCCTAGGTTTGTTCCTAGGCCTTTGTTTTTTAAGCAACTGTAAATGGGATTTCTTTTTCCTCCTAGT
TTCTTTCTCAGCAAGTTTGTCATTGGCATGCAGAAAAGCTAATAATTTTGTATGTGTTGGCTTTATTACCTCCTATTTTGCTGGAAG
TGTTTATTGGATCTAGCTGTTTCCTGATGAAATCTTAGGGTGTTGTTGTTGTTTTTGTTGTTTCCTTTGGTTAAAGGCCATGTCCTC
TGCAAATAGAGATGAGTTGACTTCTTGTTTCCTGTGTGTGTCCCTTTTATTTATTTCTTTTATTTTCTTGCTAAGATTTCAAACACT
ATATTGAACGAATTATTTATTTATTTATTTATTTATTTATTTAATAATATCCTTCCTTGTCTCTTTCCATATCTTAAAGGAAACTCT
CTAAGCTTTCTCATTTTGAACAATGATTGTTAGAGGTTGAGAGATTAACTCGGAAGCCAGGCAAACACAGACCCCGGATTCAGTCTC
CAGCTCCAAAGAGTAGTTGTAGTGATGATGATGGTGATGGTGATGCTGATGACAATATTAATATTGTCATATGTTTGTCATGTGTAT
GCTTATTAAGATAACATGCTTTTTGTATTCCTAGTTTTTCAGGATATTTGTCAGGAAGGAATGTTAAACTTTAAAACCTGTCCTACA
TCTATTGAAATGATCATGTGACTTCTGTCCTTTAAATATATTTATATGCCTAATTACTCTTACTGATTTACATGTGTAGAACCCACC
ATGAATTCCCTGCAGTGAACTACCTTGGTCATCATGCAAGATCTTCCTAATGTATTCTTGAATTAAATTGACAAGAATTTTATTCAG
AACTTTTGCATCTATGTTCATTGGGGAGTTTGGTGGCTACTTTTCTTGTGTGTGTGTCTGTGTGTCTGTTGTGTCTGTGTGCACATG
CGCACTCCTATCCAGTTTGGAGCATTAGGGTAATGCTGGCTTTACAGGATGCTTTGGGTACCACGCCTCCCTTTCTTATTCCATAGA
ACAGATTGAGAAGTATTCATGTAAGTCCGTCTTTAAAGGTTTAGTGTAATTCTGTAGTGACTCTGCCTGGCTTTCTTTGTTGGGAGA
TTGTTTCTATTAATGCTTGATCCTCAGTGATTGTTATGGGTCTGTTTAAGGTTTATTTCCCAATTGATTTAATTTTGATATGTTACA
TATGTCTAGGAATTATCTATTTTGAGATTTTCTAAGCTACTTTAGTATTATGTTCAAAGTTGCCTCTAAATATTCTCTGGGCTTCAC
TGGAGTCTGCAGTAACCTCCATTTTCCATCTCTAGTTTTATTAGGTTGGGTCTTCTCACTCTTTCCTTTCATTTGCCTCAAGGTTTA
TTAGTTGGGTGTTCTCAAAGAACCATTTTTTATAGATGCATTTGGTACCTTTATTAATTTTATATATTATTCTTTTATTCTCTGTTT
CATTAATTTCTGCCCTGATCTTTATTTTTTCTTGACATCTGCTAGTCTGGGGCTTGGATGTACATGTTTTTCTAGGACATTGAGGTG
CATCATTAGACACTGTTGGACAAGCTGGACGACAGCCTGAAAGCTATTCTAATACATCCCTTTCTGTAAATGATAGATAGATAGATA
GATAGATAGATAGATAGATAGATAGATGATAGATAAATAGATAGATAGATAGATAGATAGATAGATAGATAGATAGATAGATCCGCT
CTGCTGCTCTCAGTGCAACATCTAAAAAAAGTCAGGGTTTTTTTCCAGGAAAAAAAAAAAAGCACTTAAAATCCATATGGAATTTCA
AAAGACCATTTGTGCTGCCTCAAGAAAGTATCTATTACTGAACAAATTATTGTTGCCTAGCAGTTTCCTCTGAATTGAAACATTCTT
CCAGGAGAGGGGAGGAAGACTGTGAGACTCAGAAGGTAAACAGCCTTCTTCGTGTGTCTAGGATAGCTGAAACCCACAAGATTTATG
AGAGGCCCTCTGCAGAGGTGCAGGGGCAATAAACATCTGCTGGGGAAGGAGACTGAGGGGCTGAAAGGAGATTCCTGGGAGTGTAGA
GCTGCCTGTAAGCTGTGCAGGGAGCCCCAGGGTGCAGCTTTCTAAGTCACCACCCACTTTGGGGTAGGGATTGTGCATCATGCAGAT
GCAAGTGACTCCTCACCCAAAGCTGCTGTCAGTGACCCCACTAAGAATTCGCTGGATCACCAAGTTGGATTCGGTGCAATTGCTTCT
TTGGTGTGTTATGGCTGCCTTTCTCAGTGCACAGACGTGTGATGTTCCTCTTGTTGAATAAGTCTTAAGCCCCATTAGAATGAACAA
AGGACAGACAGCGACATTTAAAAAGTGACAGAGACACATGGATACAGAAATGCTGGGGTCAGGTGGGTCTTGTGTACTCTAATGGAG
CCACTCCTACCTCTGCCGCAATCTGGAACCTTAGAATATGTGTTGTATACAGCACAGAGAGAGTGGTTAGCTAGTGTATATAAAAAT
AGATAGATAGATAGATAGATAGATAGATAGATAGATAGATAGATAGATAGATAGATAGATAGATGATCCTGTTTTTCCCACTCAGAC
CCTGCTCCCAGAACGACGACTCTGAGACTAGTTATTAATGCCTAGGCCACAAGCTTTGGCTCATTTCCCTACTAGCTTGTAACTTAT
TCAACCTATTCTAACCATCATGTCTTCCACATGGTTAGTTACCATTCCTCAGCTTCATAGGTTCAAGTCTAGGGCAAATCTCCTCTG
TATTCTATTCTCAGTTCAGCTACCTGCTGGTTTACCACAGTCTCCTCAGCGTTCCCAGGCAATTCTCTCAAGCCTCTCACTATTTCC
CAGAATCCTCTCCCTTCTGCCCAGTGTCCCACCTCCCATTTTCTGCCTCAGCGCATTGGCCAAGGCTTTTATATTGACAGGTGTTGC
TAGTCTGAAGGAAAGGCCATCCAGATACTGCCCCACCTGGGGCTTCATCCCATACACAGTCACCAAACACACACTATTGTGGATGCC
AAGAAGTGCTTGCTAACAGGAGCCCGATATAGCTGTCTCCTGAGAGGCTCTGCCAGAGCCTGACATATACAGAGGCGGATGCTCACA
GCCAACCATTGAACTGATCACGGGGTCCCCAATAGAGGAGTTTAAGAGAAAGGACTGAAGGAGCTGAAGGGGTTTGCAACCCCATAG
GAAGAACAACAGTATCAACCAACAAGACCCCCCTCCCCCAGAACTCCCAGGGACTAAACCACCAACCAAAGAGTTCACATGGAGGGA
CCCATGGCTCCAGCCACATATGTAGCAGAGGATGGCCTTGTAGGGCATCAATGGGAGAAGAGGCCCTTGGTCCTGTGAGGGCTCAAT
GCCCCAGTGTAAGGGAATGCAAGGGTGGGGAGGCAGGAGTGGGTAGGTGGGTGAGGGCACACCCTCATAGAAGCAGGAGGAAGGGAG
ATGGAATGGGGGGGTGTCTTCTGGGGGGAATTGGGAAAGGGGATAACATTTGAAATCTAAATAAATAAAATAGCCAATTAAAAAAAG
AGAGAGTCTCTCTACAAGCTAGTCTCGTTAGGGTAGGAGATCTCTGTAGGTAAGCAGTCTCTGGGCGTTTACAAGGAGTCTCTGAAT
ATAAACATTTGAGAGGAAGAAGATAGGGTCACTGGGTTTTGCACCTATGGCTCAATCGTTCATAAACACTCCTAGTTGAGCCAGAGG
AAGACGGTTTTAACAATCCAAAGCCTGACCCATGTCATCAACAGCCATACATTCAGCACTTCACTAACTCAAGGTTTCCTCTCCTCC
CTACAGAAAGGGGAGGAGGAAGGAGCTTAAAGGGAAGAGAAATAGAAAGTGTAAATACTGAAAGCAGAGAGGGGACTATTGGGGGAG
GTGGGGCTAGCAGGTGAGGGAGCGAGGCATGGAAGGGCAGTGGGCAGCGAATGAGAACAAAGTATAATAAGTTAATGCGTGAAAGTG
TCACAGCAAAACCCATTACTCTGTACACTGACCAAAATAATAATAACTTGAACAAATATTAACTGTTGGACCCTAGTCTCACTGGTA
GAAAACCATCCATGGAATATTGATTGCCTTGTTTATGCTCCCTCTTTAACTCCAATTCCACATTCTGATGTTGCCTACATAATTGCT
ACTCTCCTCAGCAGATTGCAAGCAGCCATGTCTTCTCAACCCTATTTAAAGTGACACTGACTAATTTCCCACAAGTCCTTGGACCTC
TCAGAGGGAACTATTAAGAGATGGTCTGCCACAGGTAGCATCTCTCCTCTCCATACCACTGTCTGCGTGATTATTGTAGCCTGTGGC
TGCATCTTACTTGCTTCTGGCTCTGGGGCAGCTTTTGGCCAACCACAGCCCTCTCCTTTTATACTCAGAGGCAGAGGATCAGAGAGT
TTTGTTGTGAGATAGTGTCTGCTAGTAATATCAGAAGCTATACTCACACAGTCTTACCCACGTGACTGCCCAAACATGCGCTGAACA
AGGACAATAACAGGCATACCAAAGTGGACAGGGAAAGCCCAGGAGGCCATAGCCCTACACAAAGGACTCAGACAACGAAGGAATGCT
GACTATAAGAGAAACAGTCTGCTCCAGGGAAGAACACAGCAATGGTTAGTCAATGCCAAGGAAAACATGCATACGAGTAACATTGTA
CAGACTGAGGAATATCTACGTATAGGCATATACATAACCTGCTTGTAATAGCAATGAAAAGAGGACATGAATTTGGAAGGAGGAGTA
TATGCTACTGTTTGAAAGGAGGAACAATAGAAAGAAATGTTGTAATTATGTTATAATCTCAAAATAAGAGCAATAACTTTAAAAAAA
ATTCTAATCATAACCCAAAGGATGAGGTAGGAATTGTAAATCCATCAATGTGCAGCATTGTTCCCACCTTTCTCTCAGGTGCAAGCC
TCTTGCTGAGTTCTCTCAGGTTCGAGCCTCTTGGTGCCCCATGGCAGAAGTAACTGCTTTACAGTTGACTCTTCAGAAGATAAAAGG
CCCAGTTTCTTGTGTTTTCCAGCTTCTCTGCTATAAATATCACCACCGTAGCTGATTGCAAACTATCAGCATTATAACTGAACACAG
AGTTTGGAAGCGACCACATAGATCTAGCTCTTAGCCTGTTCCAGCTGGCCCCGGCACATGGCAGCCAGCAAATGCTAATGTTTAAAT
GTGGAGAGTAGTTATTTTCTTTTAATTTTTGAGACTGTCTCACTGTGTAGCCCTGGCTGGTCTGGACCTCACTATGTAAACAAGGCT
GGCTTCAAACCCAAGTTCTGGGATTAAAGGTACCTGGCTTATAAGGTACACTCTTTGTATACCATGGACCCTAACTAACTGCCCCTA
TTTTTTAAAATATGTATTTTTTTTTAGAATTTCGCACATGGATTTAATGCATCTTCATTATATTCCCCTCTTTCCTGATCCACACCC
CCTCCCCCACCACCCCAACTCCATTTCCTCTTTTATTACACTCACTGAGTCCAGTTTGTACTGCATATACACTCACGGATGTGAGAC
CGTCCACTGAGGCCTGGTCATGCTACCGGAGTCCACACAGGATGCCTTAAAGAAAGCGGACCCTCCCTCCCCAGCAGCTCTCACCTC
TCAGCTAGGAGTGCAGGCTCAAGATCCCCTCCCTACTCTGTGCTGGATAGTTCACTGGCTTGGTCTCGTGCAGGTCTCGTGTAGCCA
CAATAGCTGCTATGTGTTCATGTATGCAGTAGTCCCTCCCGCTGTGTTCAGAAGACATTGCTTTCCTCTGCTCCTCCTTGACTGATG
GCTCTTACGATCTTTCTGCTCTCTCTTCTACAATGACTCTGGAGCCTTGGGGGTGGGAGTGGACGACGTCTCAGTTGCTGAACACCC
AACAGACACTTATCCCTGCACTTCAGTTGTGAGGTTCTGCATTAAGCACTGGTCACTGCATCGTGAAACTTCTCTGGTGAGGTTTAA
GAGATGCTCTGATCTATCGATATGGAGCTATGGATTTAGAGCCTGTCCCTATTTTATAGGAGGAAGTGGCCATCTGCTCTAAAGCAG
AAGGGAAAACATGCAAAGAGAGTACGTTGCTCTCCGGCACAGCAGCACCATTGTGGAAGTCACAAGAATAATTTTAAGTATGATTTC
TACCAGAAACTCTTTAACTTCCTTTCAATGGCCTTAAGAAAGAATTAGGAACTACTCATGTAGTCCCGCAGGGTGGGGCTGAGGTTA
GGGTTACAATATTATAATGAAAGACCTTTCTAACACAATGTTTTCTCCTTTGCTGTCCCACACGTTACCGATGTTCTCAAATATCTG
TCCCCTGAGTGTAATCTAAAGCCACTCGGGATCCAGATAAAGTAATCCATTTCCCACTCTTACTACTAAACTGTATAATCCTAAACA
GATCTGTGACCACCAATGATTCTGGCAAATGAAGGCTTCCCTGAAGATCTGCTGGGCAGATGAAGTAAACGGGTTGTAAAAGCAGCT
TCCACTTCCAGGAACAGATGTGCTGAGCCACAGGCATGCAATATTGCCACCTTGTGGCTTCTCACAGAATTGCCCAAAAGGCAAACA
TTCCTTGGAGGTCCAAAAGGAACCTTACTAGGGGTGTGGGGAGTGCAGTCTAGAGTTGTGATGAGATCCAGAGCTATGATTATGAGA
AGCCAGTCGGTATCACAGGAAGAGACTGGCCTCTACCAACCACCATAAAACTCATTTGGGGAGTTGTTGTTTTGTTTGGACAATTAT
CAGCTCTTTGGCCCCCAAAGACAAAATTATCTAAATATACATTTTACAGTTGAAAGGAGAAGATAACAGTTCCTACAGTTTGTTCAA
ACACTCGTGATTATCGGCTGCCCTTCATATTGTTCTTGCCCCTCTTTCCTATTCCAGATTCCCAAGAATAGAATGTGCCTGACTCCC
ACAGTACCCTATAGATCTGGCCAATCATCTCAGTTTTCAGTGGAGCCATGGAAATGCTCCCAATACCCTCCATCTCTTAGAAGAACA
ATCTCAGGTTCCAATCTCGTCCCCGGTGCTTCAGATCCAAACCTCCAAAATTTCACATTATTTAGTAACAGATTTGGTGGCACGCTC
CTGACAAACTCAAGTCAAGAACCCCCGAGGAAAATGGCAGACTAGAAGACGTTTCTACGCCACCCTTTGAATGAGAAGAGCCACAAT
AAGAAAAATAGACACTTCTGAAGAGAAAGAAAGAAGAAACGTTTTAAAATGCACCAAAATAGTGTTAAAATACCTGGAAAGTGGGGA
GGAAAGACAGGTAACGCATGAGAAACAGCCAACGTAAACACAGACCAGCAGCATGGCTGTGATGTGGACAGGGTGAACAGACTTCTC
ATAAGCTAAAGGAAGCTGCCCTGGTGAGAGAAAACTGGCCGTCCTGCCCTCAGAGCAAGTCCTCAGCCCTCACAAGCCTGAAAACCA
GTGGCAGCTTTTGTGAGACAACCATTCCTCCGGCACTGAAAGAGGAAGCACACCCAGCAGCTGCGTGCGCCTCAGAGCGGAAGTGAC
CCGGTTTGAATGAGAAGGTCTCCCACAGGCATGTGGATTTGAACTCTTGGACCCCAGATGCTAGCACATTTGGAGAGGTTATAGAAC
CGCTAAGGTATGGAACCTTGCTGGAGAAAGCGTGCCCCTGGAGATAGGCTTCGTTAAGAGTGTAGCCTGCTTCTAGTTTACCCTCAG
CTTTCTGTTTGAGGTTGAGGATTCAATCACCCCCGCTTCCTGTTCTGGCCCCTCAGCCTGCGGTGGACTCTGCCTTTGGAACCAGAA
GCCAGAATCGGCATGTTGTTCCTAAGTGGCTGCTGACTGGCCATGCCATTTTCCTCACAGCAACAGAAAAATGTCTGATCCAGACGT
TGGTACAAAAGAGTGGGTACGG
MOUSE mRNA SEQUENCE:mR27-007.1 (Seq ID No: 12)
CAGATTTCTCCTTGGAGCTGTGAGTGACTACCATTGCGAGCAAGAGCAAGAGGAAAGCACTACCTGTGAGCAGATGTCTGGTTCATT
CTCACCCTGTGTGGTGTTCACACAGATGTGGCTGACTCTACTGGTTCTGACTCCTGTCAATGGACAGCATGATTTTCTGGTGCTGCA
AGCTCCACCTGCTGTGTTTGAAGGAGACTCTGTGGTTCTGAGGTGCTACGCAAAGAAAGGCATAGAAGCAGAGACCCTGACATTTTA
CAAGGATGGTAAAGCTCTGACATTACATCATCAAAGTGAGCTCTCTATTCATCATGCAAATCTGAAGGACAACGGTCAATACAAATG
CACTTCGAAGAAGAAGTGGTCTTTTGGGTCCCTCTATACTTCCAATACGGTCGGAGTTCAAGTCCAAGAGTTGTTCCCACGGCCTGT
GCTGAGAGCCAGACCCTCCCATCCCATAGATGGAAGTCCAGTGACCCTGACGTGTCAGACCCAGCTCTCTGCACAGAAGTCAGATGC
CCGGCTCCAGTTCTGTTTCTTCAGAAACCTCCAGCTTCTGGGGTCAGGCTGCAGCCGCTCCTCAGAGTTTCACATTCCTGCCATATG
GACTGAAGAGTCAAGGAGATACCAGTGCAAGGCAGAAACAGTGAATTCCCAAGTTAGAAAACAAAGTACAGCGTTCATAATCCCAGT
GCAGAGAGCTTCTGCGAGATTCCAAACACACATCATCCCAGCCTCAAAGTTGGTGTTTGAAGGGCAGTTGCTGTTACTCAACTGCTC
AGTAAAAGGAGTCCCAGGACCCCTCAAATTCTCCTGGTATAAAAAGGACATGCTGAATGAAGAAACAAAGATTCTTAAGTCCTCCAA
CGCAGAATTCAAGATCTCCCAGGTGAACATCAGTGACGCAGGGGAGTATCACTGTGAAGCTACCAACAGCCGCCGAAGCTTTGTCAG
CAGGGCATTTCCCATCACCATAAAAGTCCCAGTATCTCAACCAGTTCTCACCCTAAGCACAGGCAAGACCCAGGCCCTTGAGGGAGA
CTTGATGACACTTCATTGTCAATCCCAGAGGGGCTCTCCATGTATCCTGTATGAATTCTTCTATGAGAATGTCTCCCTGGGGAATAG
CTCTATACTCTCTGGAGGAGGAGCATACTTCAATTTCTCTATGAGCACAGAGCGATCTGGAAACTACTACTGCACAGCAGACAATGG
CCTGGGAGCCCAGTGCAGTGAAGCTATAAGGATCTCTATCTTTGACATGACAAAGAACAGAAGTGTTCCTATGGCTGCCGGAATCAC
TGTGGGACTGCTCATCATGGCTGTTGGAGTGTTTCTGTTTTATTGCTGGTTCTCTAGAAAAGCAGGAGGAAAGCCTACCTCTGATGA
CTCCAGAAACCCTTCAGATTCAGAACCCCAGGAGCCCACCTATTACAACGTACCAGCCTGTATAGAACTGCAGCCAGTGTACAGCAA
TGAGCCTGAGGAAAACGTGATTTACACAGAAGTACGGAGAACTCAACCAAGACAGAAACATGCAGATCAGGAGTCTGAAAGCCCAAG
ATCAAGGTGCCAGATGGCTGAGAAAAAGTAGGATATGTCTCCTCCAAGATTCCCAAGAATAGAATGTGCCTGACTCCCACAGTACCC
TATAGATCTGGCCAATCATCTCAGTTTTCAGTGGAGCCATGGAAATGCTCCCAATACCCTCCATCTCTTAGAAGAACAATCTCAGGT
TCCAATCTCGTCCCCGGTGCTTCAGATCCAAACCTCCAAAATTTCACATTATTTAGTAACAGATTTGGTGGCACGCTCCTGACAAAC
TCAAGTCAAGAACCCCCGAGGAAAATGGCAGACTAGAAGACGTTTCTACGCCACCCTTTGAATGAGAAGAGCCACAATAAGAAAAAT
AGACACTTCTGAAGAGAAAGAAAGAAGAAACGTTTTAAAATGCACCAAAATAGTGTTAAAATACCTGGAAAGTGGGGAGGAAAGACA
GGTAACGCATGAGAAACAGCCAACGTAAACACAGACCAGCAGCATGGCTGTGATGTGGACAGGGTGAACAGACTTCTCATAAGCTAA
AGGAAGCTGCCCTGGTGAGAGAAAACTGGCCGTCCTGCCCTCAGAGCAAGTCCTCAGCCCTCACAAGCCTGAAAACCAGTGGCAGCT
TTTGTGAGACAACCATTCCTCCGGCACTGAAAGAGGAAGCACACCCAGCAGCTGCGTGCGCCTCAGAGCGGAAGTGACCCGGTTTGA
ATGAGAAGGTCTCCCACAGGCATGTGGATTTGAACTCTTGGACCCCAGATGCTAGCACATTTGGAGAGGTTATAGAACCGCTAAGGT
ATGGAACCTTGCTGGAGAAAGCGTGCCCCTGGAGATAGGCTTCGTTAAGAGTGTAGCCTGCTTCTAGTTTACCCTCAGCTTTCTGTT
TGAGGTTGAGGATTCAATCACCCCCGCTTCCTGTTCTGGCCCCTCAGCCTGCGGTGGACTCTGCCTTTGGAACCAGAAGCCAGAATC
GGCATGTTGTTCCTAAGTGGCTGCTGACTGGCCATGCCATTTTCCTCACAGCAACAGAAAAATGTCTGATCCAGACGTTGGTACAAA
AGAGTGGGTACGG
MOUSE PROTEIN SEQUENCE:mP27-007.1 (Seq ID No: 13)
MSGSFSPCVVFTQMWLTLLVVTPVNGQHDFLVLQAPPAVFEGDSVVLRCYAKKGIEAETLTFYKDGKALTLHHQSELSIHHANLKDN
GQYKCTSKKKWSFGSLYTSNTVGVQVQELFPRPVLRARPSHPIDGSPVTLTCQTQLSAQKSDARLQFCFFRNLQLLGSGCSRSSEFH
IPAIWTEESRRYQCKAETVNSQVRKQSTAFIIPVQRASARFQTHIIPASKLVFEGQLLLLNCSVKGVPGPLKFSWYKKDMLNEETKI
LKSSNAEFKISQVNISDAGEYHCEATNSRRSFVSRAFPITIKVPVSQPVLTLSTGKTQALEGDLMTLHCQSQRGSPCILYEFFYENV
SLGNSSILSGGGAYFNFSMSTERSGNYYCTADNGLGAQCSEAIRISIFDMTKNRSVPMAAGITVGLLIMAVGVFLFYCWFSRKAGGK
PTSDDSRNPSDSEPQEPTYYNVPACIELQPVYSNEPEENVIYTEVRRTQPRQKHADQESESPRSRCQMAEKK*
MOUSE PANTHER CLASSIFICATIONS
              FAMILY (SUBFAMILY)
                            CARCINOEMBRYONIC ANTIGEN (FC RECEPTOR-LIKE PROTEIN)
              BIOLOGICAL PROCESS
                            Biological process unclassified (2.99.00.00.00)
              MOLECULAR FUNCTIONS
                            Cell adhesion molecule (1.05.00.00.00) > CAM family adhesion
molecule (1.05.01.00.00)
MOUSE GENE ONTOLOGY
              BIOLOGICAL PROCESS
                            phagocytosis > phagocytosis, engulfment
              MOLECULAR FUNCTION
                            GO molecular function > cell adhesion
              CELL COMPONENT
                            cell > membrane fraction
MOUSE PROTEIN DOMAINS (INTERPRO SIGNATURES)
                            IPR003598 (IGc2)
                            IPR003599 (IG)
                            IPR003006 (ig)
MOUSE mRNA SEQUENCE:mR27-007.2 (Seq ID No: 14)
CARGTTACACTCAACTGTTTTAGARGAGCAGTTCCCCAGATTTCTCCTTGGAGCTGTGAGTGACTACCATTGCGAGCAAGAGCAAGA
GGAAAGCACTACCTGTGAGCAGATGTCTGGTTCATTCTCACCCTGTGTGGTGTTCACACAGATGTGGCTGACTCTACTGGTTGTGAC
TCCTGTCAATGGACAGCATGAAGCTGCACAGCAGTCTGTGGTTTCCCTTCAGCCTCCATGGACCACTTTCTTTCGAGGAGAGGTCGT
CACACTGACTTGTTATAGATTCGGCTTCTCCGTACCCCAGAAAACAAAATGGTACCAGAAAAGAAAAACAGTGAAGCAAACCCCAGG
TGCTTTGGTAATTAAAGCACATACCTTAAAGGTCCATGAGTCCGGAGAGTATTGGTGCCAAGCCGACAGCTTACTTCCGAGCATGCA
CGTGAACGTAGAGTTTTCTGAAGATTTTCTGGTGCTGCAAGCTCCACCTGCTGTGTTTGAAGGAGACTCTGTGGTTCTGAGGTGCTA
CGCAAAGAAAGGCATAGAAGCAGAGACCCTGACATTTTACAAGGATGGTAAAGCTCTGACATTACATCATCAAAGTGAGCTCTCTAT
TCATCATGCAAATCTGAAGGACAACGGTCAATACAAATGCACTTCGAAGAAGAAGTGGTCTTTTGGGTCCCTCTATACTTCCAATAC
GGTCGGAGTTCAAGTCCAAGTCCCAGTATCTCAACCAGTTCTCACCCTAAGCACAGGCAAGACCCAGGCCCTTGAGGGAGACTTGAT
GACACTTCATTGTCAATCCCAGAGGGGCTCTCCATGTATCCTGTATGAATTCTTCTATGAGAATGTCTCCCTGGGGAATAGCTCTAT
ACTCTCTGGAGGAGGAGCATACTTCAATTTCTCTATGAGCACAGAGCGATCTGGAAACTACTACTGCACAGCAGACAATGGCCTGGG
AGCCCAGTGCAGTGAAGCTATAAGGATCTCTATCTTTG
MOUSE PROTEIN SEQUENCE:mP27-007.2 (Seq ID No: 15)
MSGSFSPCVVFTQMWLTLLVVTPVNGQHEAAQQSVVSLQPPWTTFFRGEVVTLTCYRFGFSVPQKTKWYQKRKTVKQTPGALVIKAM
TLKVHESGEYWCQADSLLPSMHVNVEFSEDFLVLQAPPAVFEGDSVVLRCYAKKGIEAETLTFYKDGKALTLHHQSELSIHHANLKD
NGQYKCTSKKKWSFGSLYTSNTVGVQVQVPVSQPVLTLSTGKTQALEGDLMTLHCQSQRGSPCILYEFFYENVSLGNSSILSGGGAY
FNFSMSTERSGNYYCTADNGLGAQCSEAIRISIFX
MOUSE PANTHER CLASSIFICATIONS
              FAMILY (SUBFAMILY)
                            CARCINOEMBRYONIC ANTIGEN (gb def: immunoglobulin superfamily receptor
translocation associated 2 [homo sapiens])
              BIOLOGICAL PROCESS
                            Biological process unclassified (2.99.00.00.00)
              MOLECULAR FUNCTIONS
                            Cell adhesion molecule (1.05.00.00.00) > CAM family adhesion
molecule (1.05.01.00.00)
MOUSE GENE ONTOLOGY
              BIOLOGICAL PROCESS
                            phagocytosis > phagocytosis, engulfment
              MOLECULAR FUNCTION
                            GO molecular function > cell adhesion
              CELL COMPONENT
                            cell > membrane fraction
MOUSE PROTEIN DOMAINS (INTERPRO SIGNATURES)
                            IPR003598 (IGc2)
                            IPR003599 (IG)
                            IPR003006 (ig)
HUMAN GENOMIC SEQUENCE:hD27-007 (Seq ID No: 16)
AATTCACTAATGCATTCTGCTCTTTTTGAGAGCACAGCTTCTCAGATGTGCTCCTTGGAGCTGGTGTGCAGTGTCCTGACTGTAAGA
TCAAGTCCAAACCTGTTTTGGAATTGAGGAAACTTCTCTTTTGATCTCAGCCCTTGGTGGTCCAGGTCTTCATGCTGCTGTGGGTGA
TATTACTGGTCCTGGGTGAGTAAAAGGGCTCACCATGGGCCCTGGGTGGGATCGGGACAGCTTCTCTCTCCACCGCAGGCCTGGTCC
TTTCTTGGGGCTCAGCCCCTGCTGAGAGACTCTCTCCTACATTAGGAGACTGTATTTTTGAACTTTACTCTGTAGCTCAGGGCTTTC
TCTTGAGGCTGAAGAAAATGGCCACAGCTGGAAGGAATTTTCAATCTTACAGAGTAGGAAACCACAATAACCACAACAATTAAAATT
TCATTTATTTTTTTCTTTAAAAAGTACTTCTTAAAGTCCAGAGAATGTTTCATGTTTGTTTGTTCATATTGTCTTACTCATAGAAGA
GCCCACTCTCCTCTCTGCAGAGGAGATGTATTTATTCCTTGTATGACAAGGCCAGATCCTGGTCCCCAGCATAAGAGGCAAGGTTTC
TAACGGGAAAGACAGGTCTTTCTCTTGTACATAATTGCAAACCACTGCAGCAATTGGCTGATTAAGAACATGGGGTGGAGGAGGTCG
CAGAGGCGTAGGTGGACAAAGCAAGGATAAATCTAAAGCATATGACTTTCTCTTTGTCCCTGTGGGTGTACGTAAGTCTCTATCAAC
TCATATGTGCGAACTGGTTGATTTTTACTAAAGGTTAAATATTATAATTAAATATGTTTATTTATTTGTTCAGCAAATGTGTTTAGT
AATATCTATGCACAGGTGCTGAGCAATTAGAGTCCATAGGAGACACTACGTCTATGTTCTGGAAAGTCATGACATAGAAGGGCTCCC
AGCAAGGCCAATGTACTTTCAAGCAGCTATGCAACAAGCTCCCAGTTAAATAATTGTCCAGGCAAAGTTACCAATGAATGCTGTAGG
CAGATAATAAACTGTATTCAAAATAGCTAGTAAAGACTTCATGAAACTTAGAGGTGAGTAGGAGCTAGAGGAGCAGAAATGGAGCAG
GCGTGATATAAGAAAAGAGGACCTCACAGTTGGAAGGTGCATGGACTATAATTCCTGAAGTAGGAATACATCTCAGTGTGGCCCCAC
ACAATGTGGGTAATGGCCAGGTCCTGAGCATGAGAGGGCAAAGTTTATAACATGGGCTCTGGGGTCAAACAGGATCTAGGTTCAAAC
TTCATCTTGCCACCTACTGGCTACCAGATTCCTGGTCTACAAAATGCAGTTAATCCTAGTGCTTACCTCACAGGATTATTGACATGG
TTATATGAAATAACCTACGTAAATGCAGACCATATAGAAACCACTCACAAAAATATTAGCTTCATTTAAAACAATGCAAAGATGAAG
GAAGCCAAAATATTCAGGTGAATGGTGAATAATTTGTTGTTCTCAGTAAAACACTTAAGTTTGGATAAGTCTCCAATAGCCTCATGT
GGACAGGGATGATTTGTCTCTCACACACTCCTGGATTTCTATTGCCTTGCTCTGGAGTATGATTGGCATTCCAAAAATCTGTTGAAT
AAATAAATCAACCCATATGCAGAATACCTACTCAATTGATAAACCTGCCAGTTTTGTGTCCTCTCTCTCTTCATTACCCATTTCTCA
AATCTCTATCCTTCTATTCCTATATTATTAACCTAGTTCTTTAATTAATTCATTATACAACACAAATATATTTAGTGAATATTAAAG
GAATATCTTTTATATGCCATTCACCGTTATAGGTCCTGGAGGATATATGGTGAACATAAGGAAAGTCCTAGGTCTCATAGAGCTTGC
ATTATAGAGAAGGGAAAATAAACAAGCAGATAATGAAGATAATTTGGATAAATACTCTGACAAAAATTACACATTACCATCTCTTGG
GCTGAGCATCAGGTCTAAATAATCTGAGCATCAGTTGATTGTTTAAAAATTAGGCATGATGATAATGAGCCCTTGGATTCAATGATC
TCATGAAAAGAGTATAGCCTGGAGTCTGGTGCCCAAGGGAGGCTTAATTCATGTCCATTTCCTTGCCCTGCCTTCTAATCAGTCTCC
CTGCTTCATATTGTCCCATTAAATTCTGTCCACTGACAGAGTGCTCCATCAAAGTCTGAAATTCTAACCTTGTCAATCTCCTCCTTG
AAACCATTCCATGACTCCTTATCACTTTTATAATAAAATTAGAACCACTTACTCAGGCCCTGCATGCTTCTCTGGTCTTATTTCCCC
CAACACTCCCAGTTCCTGAGCACCAATGTCATTTCTCCACTTCTGTGCCCTTTCTACCTTGAAAGGTAGAAATCGCCTCTTACCACA
CACAAATGCTAACATCCACCTGCAGACTCATGTTCTCACTCACAGTTTTGTGAAGTATCATCTCCTTCAGGTACCTTCAAGGATCCA
CCCATCCTGTTCAACACATGTACACACAAACACACACTCTACACACACACATATATCCTACACACTGGGCTAGTGGCCCTTCTGCCC
CCAACATACCCCATGTGTATTTCTGTTATTGCACTTACACCCTGTGTTATATTATTTTAAATCGTGTTTCTGTTTTTCCACTAGTCA
ACTTCTTGATGGCAGATACCATTTTTAAAATTCATTGTAGAAGTCCAAATACCATGCCATATATATATGTATATATATGTATATTTA
TGTGTATATATATACATTTGATGAATAATTATTAGAGTTTTTAAATGAATTTTTTAAAGGGACAAGTAATACATGCATGGGTAAAAA
AGGGAATTATCTTCTTAAAACAAAATAATCATGGTTCTGTGCTCATCTCTGGCTCTTATTTTTTTGCAGCTCCTGTCAGTGGACAGT
TTGGTGAGTTCTCCTGTCTTTGGCTCTTCAGCGTCTCTGGTTAAGAGTGTCTCTTCCTAATAAATAGAAAACATTTGTGATGGCTGC
CTGAGATCTCCAATATCTCCTGTTTTCTTTCCTTTTTTTTATTATACTTTAAGTTTTAGGGTACATGTGCACAACGTGCAGGTTTGT
TATATATGTATACATGTGCCATGTTGGTGCGCTGCACCCACTAACTCGTCATTTAACATTAGGTATATCTCCTAATGCTATCCCTCC
CCCCTCCCCCCACCCCACAACAGGCCCTGGAGTGTGATGTTCCCCTTCCTGTGTCCAAGTGTTCTCATTGTTCAATTCCCACCTATG
AGTGAGAACATGCGGTGTTTGGTTTTTTGTCCTTGCAATAGTTTGCTGAGAATGACAGTTTCCAGCTTCATCCATGTCCCTACAAAG
GACATGAACTCATCATTTTTTATGGCTGCATAGTATTCCATGGTGTATATGTGCCACATTTCCTTGATCCAGTCTATCATTGTTGGA
CATTTGGGTTGGTTCCAAGTCTTTGCTACTGTGAATAGTGCCGCAGTAAACATACGTGTGCATGTGCCTTTATAGCAGCATGATTTG
TAGTCCATTGGTTATATACCTGGTAATGGGATGGCTGGGTCAAATGGTATTTCTGCTTCTAGATCCCTGAGGAATCGCCACACTGAC
TTCCACAATGGTTGAACTAGTTTACACTCCCACCAACAGTGTAAAAGTGTTCCTATTTCTCCACATCCTCTCCAGCACCTGTTGTTT
CCTGACTTTTTAATAATTGCCATTCTAACTGGTGTGATATGGTATCTCATTGTGGTTTTGATTTGCATTTCTCTGATGGCCAGTGAT
GATGAGCATTTTCTCATGTGTCTTTTGGCTGCATAAATGTCTTCTTTTGAGAAGTGTCTGTTCATATCCTTCACCCACTTTTTGATG
GGGTTGTTTGAATTTTTCTTGTAAATTTGTTTGAGTTCATTGTAGATTCTGGATATTAGCCCTTTGTCAAATGAGTAGATTGCAAAA
CTTTTCTCCCATTCTGTAGGTTGCCTGTTCACTCTGATGGTAGTTTCTTTTGCTGTGCAGAAGTTCTTTAGTTTAATTAGATCCCAT
TTGTCAATTTTGGCTTTTGTTGCCATTGCTTTTGGTGTTTTAGACATGAAGTCCTTGCCCATGCCTATGTCCTGAATGGTATTGCCT
AGGTTTTCTTAAAGACTTAAATGTTAGACCTAAAACAATATCTCCTGTTTTCTTGCTGAGGTCAGCAGGGATTTCAAAGTGATGCCT
GGTTTTTTGGCATCGTCCAAGGGCCAGGATAGCTTCATGGTCACCCTCTGACCCTCCCTGGGGGAAGGGGTGAATGCCCAAGCAGCT
CCCAGCCTTTGCTAAGCCCTGCATTTACTATTATTGCCAACAGACCCCATTTAGCCAGGCAGGACATCTTCAACTCCATGGCAAGTA
TATTGACTCAGGATAGGATCCTCTCAATCAGCGAGCCAGAAAAATACCCTGAAAATATCCCACTGTGCTGATGCCAGCAGGAATGCT
GCATTCTCTACACTTTGGAGTAAAATCCTCCAGGACATCCAATAACTGGGTCCTTACAAAAAAGAAAGGCACAGAAAGGGGAGATGG
GGAGAAGACAAGTTATCTGCATCCCACATTTAATTAGTTCTGGCTCTTACTGCCATTCCTTTGGCTCTGAGTGGATAATTGAGAACC
CATGAACAGCAACAACCCTCTACAACCCCTCTCCTTGTCCAAATCAGTGGCCCCAGCTGTAACTTTGTCCTGGTCACCTCTGGATGT
CAGTGAGTGACTTGTCCCTTCCTTGCTCTGCCACAATTAGCTGTGATGGGATTCTTCTGGGCACTTGAATCATCATCTACAAAATGA
AGATATTAGGTAAGTTGAGCTCTCAAGGTTTTCTCAGGGACAAATCTTAAGATAATATGATTTATTCTCTTAGGGAAAGTGCAAAAT
TGTGATTTTAACATAACCTTTGGAGTTTCTTATGAGTGTATGAACATGATGAAATGGCAGCGCAGCATTGATTTTCTCTGGGACCCA
CTTCCATGTAAGGCTACCTAACTGTGATATTTGATGGCGAATCCTGCTGGTGTCACCCTCCTGAAATCATTTTGCTCATTGCCATCC
CCTGGGCAATTTCTCCCTTGGGTGAGGTGAGGGCCACTGGCTGACAGGGAGCTTTGAGCTATCAGTGACCCTCCCAGAGGACCTCTT
TCAGATCCTCCAAACCCAGGTCCTTCAGGCTATGCAGCAGGAGGCCCTGAGGCTGGGTCTGCAGGCGCCTCCACCTCAGACTCCTTT
TCTCTTTCCTCTTCAGCAAGGACACCCAGGCCCATTATTTTCCTCCAGCCTCCATGGACCACAGTCTTCCAAGGAGAGAGAGTGACC
CTCACTTGCAAGGGATTTCGCTTCTACTCACCACAGAAAACAAAATGGTACCATCGGTACCTTGGGAAAGAAATACTAAGAGAAACC
CCAGACAATATCCTTGAGGTTCAGGAATCTGGAGAGTACAGATGCCAGGCCCAGGGCTCCCCTCTCAGTAGCCCTGTGCACTTGGAT
TTTTCTTCAGGTGAGAGAGCGTGAAAGACGCACCAACTTTAGAAATCAATTCAGCCCATGTTTCTCTCTAGCTGTTATTACTGTTTA
CCTCAGACCCTTCATGAGTGGACAATTTTATTTCACACTGTGGTAAGACAAGGAGAGAGTAAAAAGCTCAGAATACAACCAGACAGG
GAATGGGGTTAGCTGCTTAATTTTAAACATTAAAATAAATAGAATTTTTACTTGAGTGGATTAATGTCTCTAAACCTGTGGTTCTTA
AAGTTTGATTTGAGTCAGAAAGCCTGGGCAGCTTATCAACCATACAGATACTCAGGACCTACCTATGCTTATTCAATAGAAACCTGT
AGAAGTGGGGTCCAGCAATTTATATTTTTTACAAGCCTCTCCGGTACCAGAAAGTCTGAGGATTAATTTATTTGAGTGGTTCCTGAA
TCTATGTATAGCTTTTTGGTTTGGTTTGGTTTGGTTTGGTTTGGTTTGGTTTGGTTTGGTTTGGTTTTTTGAGATGGAGTCTCGCTC
TGTCACCCAGGCTGGAGTGCAGTGGCGTTATCTCGGCTCACTGCAACTTCTGTCTCCCGGGTTCAAGCAATTCTCCCACCTCGGTAT
CCCTAGTAAGTAGGACTACAAGAGCCCGCCATCACACCTGGCTAATTTTTGTATTCTTAGCAGAGATGGGATTTCCTCATGCTGCCC
AGGCTAATGTTGAACTCCTGGGCTCAAGTGATCTGCTCACCTAGGCCTCTCAAAGCGCTGGGATTACAGGTATGAGCCACTGCACCC
GGCCTCTATGTATAGTTTTAAGTCACATTATTTACTTGGAATAATAAGTGGCAGGAGAATTGTTCATACTTATGGGTAGATAATGCA
TAGGTAGATAAATATACAGAGAAAGAAAGATAAAGGAAGAGACATTTCCTTCCTTGTCCTAAAGCCACCTGTTTTGAAGTTGAAGTT
ACATCATTTTTTAATGTAGTGGAAGCAGTGTACTAGCAAGGAATAAACAAAAATGTTTATACTCTTTGACTTAACAATCCGTCTCTT
AAGAATTTGCCTACTGAAACAATCACAGATGTCCATCCCTGAAAATGGCAGACCTTAACATGGTTTAGAAAAAGAATACCTGGCCAG
GCGCGGTGGCTTACGCCTGTAATCCCAGCACTTTGGAAGGCCAAGGCGGGTGGATCACGAGGTCAGGAGATTGAGACCATCCTGGCT
AACACTATGAAACCCCGTCTCTACTAAAAATACAAAAAATTAGCCGGGCTTGGTGGCGGGTGCCTGTAGTCCCAGCTACTCGGGAGG
CTGAGGCAGGAGAATGGCATGAACCCGGGAGATGGAGCTTGCAGTGAGCCGAGATAGCACCACTGCAGTCCTGCCTGGGTGAAAGAG
TGAGACTCCGTCTCAAAAAAAAAAAAAAAAAAAAAAAACTCATTAAAGAAATTACATTACTGCCAGTGAAGGAAAACCGCAAACATT
AAAAATCAGGTTCAATAATGATACTAATGATGTAGGAAAGTGTTCATAGCATAGTAAGTGAAAGGAAAGTTATCAAATCTACAAATA
TTAATATTATTCCATAAAAGAAATACGCATGAGCTCCAAGTACTTGAAAGTGGAAGAGAAAACTGGAAAAACATATGCCAAAACATG
GGTGGTGAGATGGTAGGTGATTTTACCTACATTTCTGCTTTTCAACTTTTCTTAAGGTGTATGTGTTCATTTTTAAATCAGAAAAAA
AGAAATGATAATTATTTTAAGAGTTCTTTTTCCCAACCCATTTTTTTAAAATAAAATCTACTTGGAAAAACAATGAAAAGAAAGGGG
AAACAGTAGGATGAACCAAATAACTCTATTGTGTTTCTCATAGCTTCGCTGATCCTGCAAGCTCCACTTTCTGTGTTTGAAGGAGAC
TCTGTGGTTCTGAGGTGCCGGGCAAAGGCGGAAGTAACACTGAATAATACTATTTACAAGAATGATAATGTCCTGGCATTCCTTAAT
AAAAGAACTGACTTCCATATTCCTCATGCATGTCTCAAGGACAATGGTGCATATCGCTGTACTGGATATAAGGAAAGTTGTTGCCCT
GTTTCTTCCAATACAGTCAAAATCCAAGTCCAAGGTAAAGCCTTCATTATTTTGTGAAGTGAGTTGGTCAAAAGGAGATTCCTTGGG
GTCATAGAGATAGAACAGGAGGAAAAGGGTGGACTGGAATACCACTAAACCACAGGACACATTCCTGCAAGGAGCAGAGGAAGTATA
GTAGTGTTTTGGGCTGAAGGAAGCTAGGGATCAATGGCATTCCTTTCTTTGCCTGCTTTTGGATGTGCTGCTGCTTGAAGTGTGCTT
TTCTCCAAAGCAGTTCCAGCCTCATTGCTCTGGGACTGTTGTGATTCTTCTCTCTAGAGCCATTTACACGTCCAGTGCTGAGAGCCA
GCTCCTTCCAGCCCATCAGCGGGAACCCAGTGACCCTGACCTGTGAGACCCAGCTCTCTCTAGAGAGGTCAGATGTCCCGCTCCGGT
TCCGCTTCTTCAGAGATGACCAGACCCTGGGATTAGGCTGGAGTCTCTCCCCGAATTTCCAGATTACTGCCATGTGGAGTAAAGATT
CAGGGTTCTACTGGTGTAAGGCAGCAACAATGCCTCACAGCGTCATATCTGACAGCCCGAGATCCTGGATACAGGTGCAGAGTAAGT
GTTGGTGGAACCTGAGATTTGCTGCAGAGAGGGCTGGAAAAGTGGGACAGGGCTGCATATCAGCGTGGGTCACCAGTCTCTGTAGAA
AAAACTCGTACCTGTGAGAATGGGAGTTGTGCAAGCAAAGAGACCTCTACTCTTGTGAACATCTTAGAAGAGCTTAGACCCAGCAAA
ATCCAAGAAAAGAAAGGTCTAATTTTTTTTCTTTCAATGCCATTAAATACAATTCTTTCTTTTCTCTCGCAGGGTTCTCTTCCCTGA
CATCATTTTTTTTTCTTCTAGGCCCATAGGGATAAGCCATGGGCTGTGGCATAGCTCTCTGACTCTGGGGTACAGAGGTATCTGTTG
TCCCAATTAACATTACTTATTGACAATAACCAAAGTGAAAATTTCTGTAGGATACAAAGACAGGTTGCAACATTATAGTTACTCCAA
ATATGTGACTCTGGAGGTTTACCATTCCCTAGTTATGAGTACAAATATGCACCCCAGATTACATCACCTCCCTTACTAGTATACAGC
ACCATCCTTCCTTCAAAGCTATTATGGTAGTTACAGTGATGAGGGAAAAACAGCTTCTGTAGAGCCCTGTGTCATCCCTCCACTCAT
GGCAAACTCCGGGCTCACTTATAAGGAGGCAAGGTGATCATCACCAGACTAGGAGTCAGGACACCAACTCTGGCCCTGGCTGGCCCT
GAAAACAGCCTACACAGGCAGGTCTCCTAGTCTCTCCTTGACTCAGTTTTCTCACCTGTAAATAAAGAAGGTCAGTCTGGGCCAAGG
GAGAGAGTAGGAGCAGAAGAAAAAGCTCTAGAAGACTATGCTAATCTTTAAGGAAAAACTAGCAATGGCAAATAAATCCCTGAGGCC
AATGCAAGTGAAGGAGAAATAAACAGGGATAGGAAAGAAGACTCAGAATAATTGGGCACTTCAGGTGCAACATTCCATAGTCATAGG
GCAGAAGGAAGGAGCAAGAAGCAGGTGCAGGTAAGGGTGTGTGGTTCTGATGGCAGCAAGTTAAGGGTGGAGACTGGAGACTGGGCA
TTTGCACTGGCCATGCCTTGTAATCTACAGTAAGAGGCAAAAGCAATTCTTGACTAATTTTTTCTCTTGCTCAGTCCCTGCATCTCA
TCCTGTCCTCACTCTCAGCCCTGAAAAGGCTCTGAATTTTGAGGGAACCAAGGTGACACTTCACTGTGAAACCCAGGAAGATTCTCT
GCGCACTTTGTACAGGTTTTATCATGAGGGTGTCCCCCTGAGGCACAAGTCAGTCCGCTGTGAAAGGGGAGCATCCATCAGCTTCTC
ACTGACTACAGAGAATTCAGGGAACTACTACTGCACAGCTGACAATGGCCTTGGCGCCAAGCCCAGTAAGGCTGTGAGCCTCTCAGT
CACTGGTAAGCCCTGCATTCCTGCCAATCACCCACCCCTGGCCAAGAGTCCTGCCTCACCCTGCCCTTCGGAAGTCAGCATATACCT
TCCAGTATCCTCAGTTTCTTTCTTGGGAAAACCTAATGTCCTCCTCCACTCTCCGGCTCACTGAACCAGCTGTCTGCCTCTTGAGTC
TCGTTGCATTGCAGTCTAGGTGCCCTCATACCCCTGCCTGATGGTGCAGTGGCTTTCTATCCCCTGGTTCCCAATTTCTGAGAAGCC
AAGTGCTTTTCTTGTGCTTTCCACTTGTCCTGTGGCTCCCCAGATGACTGATTTTCATTCACTATATTATTCTCAGTAGACACAGAC
TCAATTTCCAGGAGCACAGAGGTATCTCCTGTTCTGTTAATATCAGACATTAATCAATAAACCAAAGGCAAAGACATGTAAGACACA
AAGACAAGTCGCTGGATCATAGTTAACCCAGCATGTCCTTCTTGCTCAAGCCTTTTTTAGAGTTGATGGGCAGTAGGTAAGAAAGGC
ACGGGCAATGGTGGGCTGGGGGCAGGCCAGGAGATAGCTTCACCTCTACTTGCTTGGCTTAATGCCCTCAGAGCTATTCAGGTAGCT
GCCAAATAGTTATGACATCAAAAAGGTTTCCTTTGAGCTTTGGATCTCAGTTTCGAACCATCCATGCATGGCTGAATGCCAGGTCCA
ATGTTGAAGGAGACAGTGTGATTTGGGTCTATACTGGTGACAGAGAGTCCAGTCTTGGTTGGTCCTGGAAGCGGCATCAGTTTCCTT
TCCTGTAAAACAATAATACCAGCTTGGAAGACAAGCAAGAATCCAGTCCCTCCCACATGGATGTGTGAGATTATAAAGGAGAGGACC
AGGGAACCCAGCGGGAGACATCAAATGACGTAGAAAGAGAACAGTGTCCCTGACATTCAGTGGCCCAAAGAAAGTGGAGACAGTGAC
ATCATGATCAAACATGCTCAGAGATGTTTAAAGTCCTATTCAATGACTCACTTCTTCACCTAATAAATGCTGATTGCATATATTCCA
TGGACGAGACTGTACTGACATCTAAAGATGCATCTACGACTACACTAACCTGGCCTTCCCCACACAGTTCGTAGGGGCTAATAGCAA
ATGCAGTCAAGTAAGAGATTAAACATCAACCTGAGGATAGAAGCACCTGGTGGCTGCAGGAGCAGATGGAAAGGCATGCCAATCTGA
GTTGGGAAGTGAGAAAAAATTTCTTGGAAGAACTCATGTGTGAGTTGAGACCTGAGGATGAGTGGGAACATTTCAGGAGAAGAGGAT
TGTGGTGAGGGAGGGAAGGCATGTTCCAGGAACTGGAGGGGCAGTCGGTTCTTGACGACTGCAGCACAGACTGAACAGTGGAGAAAG
GTAGGATAAGGTTGGAGAGGAAGGTATGTTTAGACCATATTTAGAAGCAGAGTAAGGCCTTGGGCCATATTTTGCAGGGCAATCTGG
GCTCCATGAATGCTTTAAATCTTGGGGAAAACCACTCCAGCAGCACAGTGAGAAATGAGTCATAGAGCAGCCAGTTTGGGGGGCAGC
CAGGAGTAAAAGAGAGGGTTCATCAAGAGAGCGTCCAGTTGGAGCAAACCCCAGGCCAGTCCCAGTAGAGGAGTTGGGACTTGAGAA
GGAAGGACTCCGGTTGAAGCAAGAGGCACTGTGAGAGTGTGCCCCAATGTCACCAAGGGCATAATGTGTTCTAAGCAGAAGAGAGGG
AGCTGGTTTGGATAAGGCAAAGCTAAGAGATGATGACAGATAAAAACTGAGTCACATCCAGTGAATTTAGCCACAAAGTACTCTTTG
GCAGAAGACAGACTAGAATCAGAATACCTGGGTTTGACTGCTGCCTCTTCACTTACTAGCTGTGTGACTCACCCACTCGGGGCCTCA
GCATGTTCATCTGTGAAAGAGGGGCAACAATAATACCCACCTCAATGAATTGCTTAGATGATTAAATTAGAGTCTCTCTATATATCG
ATATTTACAAATTACATCCAACAAATATACAAACACCAGAACTTACTTAAAGACACTATAATGTCATTATAACAAACATCTATTTTA
TTATTTTTATCTTTATCATCATAATTATATTCACAAACATGAAAATCCACTTTAGTAGATTGTTGGGGATTAAAACCAAGTTGTAAT
GGACAAAACATCCATATGAGATATGAGATAAAGAAGTAGAGATGTTAAAGGTAGGTCACTTCTTCCAGAAGACTGACTACAGAAAGG
TGGACAAACCGACTGTGAGGAACTGCAAATGCAAATAGGGTTGTTTTTCTATTGTCTGATGGTTTTTTTTTTTTTTAAACATGGAGA
GGGTTAAACACATGCAAATGGTGTTTGCAAGAAGCCAGTAGAGGTAAAAACCTTTTATCTAGGACAGAGGCAGGTGAGCACAGGATG
AAAAGACAGAAGAAAATCCCCCCAGGCAGGCCAAGGAGGGCTCCTCCATAGCCTCAGGGCCCAGGTCAGAGTGAGGAGCTGATGCAC
GTAAGGGTGTGGTTCTGCTGGTGGAAAGACAAGGGAATTCTTTGAGAGCAACATCTCTGCACAACAGAGGCAGGTCCTACAACAAGG
GGAGCTGGGAGAGGAGAAAAGACAAGAGGTCTGGTGGAAAAGGAAAAGACAGGGAATGACTAGGATGGAGGACAGGAGAGCTGGTGA
GAGTAGCCTGGGAAGTGGCTGGGTCACCCTGCAGGTGGGTAGAGCCTGTGGACTGGAGACTGAACAACTGCACTGGTGAAACATTGA
ATCTGTGAACCAAAAACTATCTGAGACAGGTCTCAATCAATTTGGATAGTATATTTTGCCATGGCTAAAGACATGTGCCCCAGATTC
AGCTCCCTGCCTTCTCCAAAGATGATTTTGAGGGTTTCAATATTTAAAGGTGAAAGGGAAGATATGGAGAAAAAGGAAGAAATTTTT
TTAAAAGTATGTGTAAATAACAGACAAACAGTTGCATTCTTTTGAGTCTTTGATCAACCTTTAGCAGAATACACAATTTACATGTGA
GAGAGGGGCAGAGGAATAGTCACTTCTGTCTTCCTTTAGCCCAGTGAGTCTGCATTTTTAAGTCAGGGAAATAATCAGATATGCATT
TGTCTCCTGTAAGCAGCAATGACTTAAAGTTCTGTCTTTTGTCCTGCACTTGTGAAAATCAGCTATCAATTTACACTGTTAGGGTAA
AACAGAACCATTTAAAACAGAACCATTTTAGGGTAAAGATCTTGGGGCCCACAAATAATATTTCAGTGGACAAATTTTGAGGGAGGT
ATGTAGCTTTTAGAAAAGTCTTTGAAGTTATCTTATTAAGGAATAAAATAAAAAGCAGGTTTGCCTGAAATAGTTCCCAGCTTGACT
TTTCCCTTTGGCTTAGTGATTTGGGGTTCCCAAGATTTATTTTCCTTTTATGAAAACTACAGCAGGAGGCAGAAACAAATCTTGACT
AATTTTTTCTCTCTCTCAGTTCCCGTGTCTCATCCTGTCCTCAACCTCAGCTCTCCTGAGGACCTGATTTTTGAGGGAGCCAAGGTG
ACACTTCACTGTGAAGCCCAGAGAGGTTCACTCCCCATCCTGTACCAGTTTCATCATGAGGATGCTGCCCTGGAGCGTAGGTCGGCC
AACTCTGCAGGAGGAGTGGCCATCAGCTTCTCTCTGACTGCAGAGCATTCAGGGAACTACTACTGCACAGCTGACAATGGCTTTGGC
CCCCAGCGCAGTAAGGCGGTGAGCCTCTCCGTCACTGGTAAGCCCTGGGTTCCTGCCAATCACCCACCCCTGGCCAAGAGTCCTCTC
TTACCCAACCCTTCAGAAGTCAGCATGTACCTTCTAGTATCCTCTGTTTCTTTCTTGAAACAACCTAATATCCTCCTCCACTCTCTA
GCTCACTGAACCAGCTGTCTGCCCCTTGAGCCTCATCCCATTGCAGTCTGGGTGCCTTCCCACCCCTGCCTAATGATGCAGCAGCTT
TCTTTTCCCTGCTCCTCAAATTCTGAGCAGCCAATTGCTTTTCTTGTGCTCTCCACTTGTCCTGTAGATCTGCCAGATGCCTGATTT
TTGTTTACTAAATTATTCTTAGTAGATATAGACTCAGACTCAGTTCTCAGGAGCACAGAAATGTCCCCTGTCCAGTTAACATCAGAC
ATCAATGAATAACCAGATGCAAAGTCATGTAAGACACAAAGACAAGTCGCTGGATCACAGTTAACCCAACATGTCTCCCTTGCTCAA
GCCTTTTTTAGGGTTGATTGGGCAGTAGGTAAGAAAGGCACAGACAGCAGTGGGCTGAGGGCAGGTCAGGAGACAGCTTAACCTCTA
CTTGCTTGGCTTAATGCCCTCACGGCTACTCAGGTAACTGTCAAATAGTTATGACATCAAAAAGGTTTTCTTTGAGCTTTGGATCTC
AGTTTTGAACCATCCATGTGTGGCTAAATGGCCAGGTTCAATGTTGAAGGAGACAGTGTGATTTGGGCCTATACTGCAGACAGAGAG
TCCAGTCTTGGTTGGTCCCGGAAGCGGCCTCAGTTTCCTTTCCTGTAAAACAATAATACCACCTTGGAAGACAAGCAAGAATCCAGT
CCCTCCCACATGGATGTGTGTGATTATAAAGGAGAGGGCCAGGGAACCCAGGGGGAGATATCAAATGATGTAGAAAAAGAACAGTAC
TCCTAACATTCAGAGGCCCAAGGAAAGTGGAGACAGGGACATCATGATCAAACACACTCAGAGATGTTTAAAGTCCTATTAAATGAC
CCACTTCTTCACCTACTAAATGCTGATTCTATATATACACCTAGATGTAGTGACATCTAAGGATGCATCTAAGACTACACTAACCTG
GCCCTGCCCACACACTTCACAGGGGCTAATAGCAAATGCAGTCAAGTAATAGAGAAAACATCAACCTAAGTTTAGAAGTACCTGGTG
GCTGCAGGAGCAGATGAAAGGCATGCCAATCTGAGTTGGGAAGTGAGAAAACAATTCTTGGAATTTGAGATGATATCGGGGGAAATT
CACCCCCAATATTTTACATAGGTTCTTTTCTATTTTCCCTAAGTGTCAGCTGGTCTCAGAAATAAAGGGACAGAGTACAAAAGAGAA
CAATTTTAAAGCTGGGTGTCCGGGGGAGACATCACATGTCGGCAGGTTCCATGATGCCCCTTAAGCCATAAAACCAGCAAGTTTTTA
TTAGTGATTTTCAAAAGTGGAGGGAGTGTACTAATAGGGTGTGGCTCACAGAGATCACATGCTTCACAAAGTAATAAAATATCACAA
GGCAAATGGAGGCAGGGTGAGCTCACAGGACCACAGGACCGGGGCGAAATTAAAATTGCTAATGAAGCTTCGGGCACGCATTGTCAT
TGATAACATCTTATCAGGAGACAGGGTTTTGAGAGCAGACAACCAGTCTGACCAAAATTTATTAGGCAGGAATTTCCTCGTCCTAAT
AAGCCTGGGAGCACTACAGGAGACCAGAGCTTATTTCATCCCTCAGCAACGATCATAAAAGACAGCCATCCCCAAAGCGGCCATTTC
AGAGGCCTCCCCTTAGGGACATATTCTCTTTCTCAGGGATGTTCCTTGCTGAGAAAAAGAATTCAGTGATATCTCTCCTATTTGATT
TAGAAAGAAGAAAAATATGACTCTGTTCCACCCAGCTCACAGGCAGCCAGAGTTTAAGGTTATCTCCCTTGTTCCCTGAACATTGCT
GTTATCCTGTTCTTTTTTCAAGGTGCCCAGATTTCATATTGTTTAAACAATTTGTGCAGTTAACACAATTATCACAGGGTCCTGAGG
TGACATTCATCCTCAGCTTACAAAAATGATGGGATTAAGAGATTAAAGTAAAGACAGGCATAGGAAATCACAAGGGTATTGACTGTG
GAAGTGATAAGTGTCCATGAAATCTTCACAATTTATGTTCAGAGATTGCAGTAAAGACAGGCATAAGAAATTATAAAAGTATTAATT
TGGGGAACTAATAAATGTCCATGAAATCTTCAAAATTTATGTTCTTCTGCCATGGCTTCAGTTGGTCCCTCCATTCGGGGTCCCTGA
CTTCCCGCAACAAGATGAGACCTGATGGAGAAGAGGATTGTGGTGAGGGAGGGAGGACACTTTCAAGGAACTGGAGAGTGGTCAGTT
CTTGATGACTGAACCATAGACAGAACAGTGGAGAACCACAAGATAAGGTTGGGGAAGACAACATCATTAGAACCTACTTAGAAGCAG
AGTAAGGGCTTGGGCCATATTCTGCAGGGCAATCTGGGCTCCATGAAGGTTTTAAATGTTGGCGAAAACCACTCCAGCAGTACAGTG
GGGATCAGGTCATAGAGCAACCTGTTTGAGGAGCAGCCAGGCGTGAAGGAGGGAGCCCAACAAGGGAGTGTCCAGTTGGACAGAACC
CCCAGTCTAGTCCCAGTAGAGGAAATGGGACATGAGAAGGAAGGACTCTGGCCAAGGGAAAAGGCACTCACTGTGAGAGTGTGCCCC
ACTGTCACCAAGGGTAGAACATGTTCTAAGCAGATGATAGTGAGCCAGTTTGGATAAGGCAAAGCTAAGAGATGATGAAAGATAAAA
ACTGAGTCATGGCCAGGAGAGGTGGCTCATGCCTGTAATTCCAGCACTTTGGGAGGCTGAGGCAGGTGGATCACCTGAGGTCAGGAG
TTTGAGACCAGCCTGGCCAACATAGTGAAATCCTGTGTCTACTAAAAATACAAAAATTAGCCAGGTGTGGTGGCGGTCGCCTGTAAC
GCCAGCTACTCAGGAGGCTGATGCAGGAGAATTGCTTGAATTCGGGAGGCAAAGTTTGCAGTGAGCTGAGATCATGCCACTGAATTC
CAGGCTGGGTGACACAGTGAGACTCCATCTCAAAAAAAAAAAAAAAAAAAAAAAAACTGAGTCACATCCAGTGAATTTAGCCACAAA
GTACCCTTTGGCAGAAGAACACAGACTAGAACCAGAATACCTGGGTTTGACTGCTGCCTCTTCACTTACTAGCTGTGTGACTCAACC
ACTCGGGGCCTCAGTATTTTCATCTGTGACATAGGGACAACAATAATACCTACCTCATTGAATTGTTTTGATGATTAACTTAGTGTC
TTTATACATATAGATATTCACAAATTACATCTGACAAATATACACACACCAGAACTTAAAGGCACTATAATCTCATTATAACAAATA
TCTTTTTTATATTTATCATTATCATCATTACTACATTCATAAACATGAAAAATCAGCTTTAGTAGATTATTGGGGGTAAAAACTGAG
TTGTAATGGACAGAATATGCATATGAGATATGAGATAAAGAAGTAGAGATGATAAATGTAGGTCACTTCTTCCAGAAGTCTGTCCAC
AGAAAAGTGGAGGAACCAACTGTGAGGAACTTCAAATACAAATAAAGTTGTTTTTGAATTGTCTGATAGTTTTTCTTATATGGAGAT
AGTTAAACACACCAAAATGTAGTTTGGAAGAAGCCAAAAGAGGTAAGGACTTTATGTCTAGGACAGAGGCAGATGTGCACAAGGTCA
AGAGACATAGGAAAATCCCCCTTGGGCAGGCCAGGCATGGCTCTTCCATGGTTGCAGGGAAGCCTGCATGGTTGCAGGAAGAAGTGA
GGAGCGGTGCAAGTGAGTCTGGTGGTGGCAAGACAAGGAATTTTTTCCTTGAAAGCATCATCTCCTCTGCAAAAAGGAGGCAGGTCA
CACTACAAGAGGAGCCATGAGAGAAGAAGAGAGTATTGGTCTCCTGGAAAGATGAACAGATAGGGAAAGACCAGCATGGAGGGCTGG
TCACAGAAACCTGAGAAGTTGCTTGGCTGCCCTCCAGGTAGGTGAAGTCTAGAGATCCTGTACTGGAGACTGGGCATCTGCACTGGC
CAAGTGTTGAAATCTACAGTAAGAGGCAAAAGCAACTCTTGATTCAATCTTTCTCTTCCTCAGTCCCTGTGTCTCATCCTGTCCTCA
CCCTCAGCTCTGCTGAGGCCCTGACTTTTGAAGGAGCCACTGTGACACTTCACTGTGAAGTCCAGAGAGGTTCCCCACAAATCCTAT
ACCAGTTTTATCATGAGGACATGCCCCTGTGGAGCAGCTCAACACCCTCTGTGGGAAGAGTGTCCTTCAGCTTCTCTCTGACTGAAG
GACATTCAGGGAATTACTACTGCACAGCTGACAATGGCTTTGGTCCCCAGCGCAGTGAAGTGGTGAGCCTTTTTGTCACTGGTAAGT
GCTGGGTTCTTGCCAGTCACCCACCCCTGGCTGAGTTCTCTCTCACCCATTCCTTTAAAAATCTGTTTGCACTGTCCAGTTTCCTCC
CCTAATCAACTTAATCCCCTTCTTGGCTTCCTCCTCAACTAACTAGCTGGGGTTTTCCGTACTCATAAGTCCTGGCTCAGCCAGACC
CCTAAAACAGCTCAGTAGATTCCCCAGCTTTTACCAAATGAATTTATTTATTGTATTTTCTCCTCATTCCTTGTATGTTCCAACAGT
ACGCCAATTTTTCTTGATGCACGGAGCGTGTCCTACTTCTCTACTGACATTTACATATTAACTTAGCTACAAGCACAGTCTTATAGA
TAAATATTGGTCAAGACCTTAAATTCTCCAAAGGATTTCCAATCTTATGGTAGATTTGGAGAAAGCTGCTGGTGAACAAAGGGGGAA
ATGGCTCCCTAGGAACCAACTCCTCAAACTTCTGGAGTTTTTATGATCCCTTGTTTTCTAACCTGCTAAAATCAGTATCATTTTATT
GTATTATTTTAAAAAAACTATTGTTGAAGTATGACATACATTCAAGAAACGTGTGCAAATTGTATGTGTACGATTTGGTGTCTTTTT
AGGAGCTAAGTTGCTTCTCTTTTTACTTGAATCTTTGTTTATAGAAACTGGGGGAAAGTTTACTTTCTTTTCAGAGAAGCCAAATGG
TATGATAGAAAAATCTTGAGCCTGATGTGTCAGACATGCCCCTAGCATAACTTGTTGAGTAAAGAGGTTATTTTTAAAATGTGAATG
TTCTGAGACTACTCCAAAGTCAGAGCCAAATCTACTAGGAAGCTTCTAGACTTCACTCATTCTGCATCCCATTACTATCTTTTTATC
CATGTTTTACTTTCTTCTCATATTCAGCAGCATCTTAAGCCTCTTTATTTTCTGTTTCTTGACTGTCACCCTTAATGCCAGTAGAAT
GTAAGCTTCATGAGAACAGAACTGCATCCATCTTGGTCTTCACAACATCCCTGTGCCTACTCAGTGTTTGGCACACAGTAGGTCCTC
AGTCAACATTTGTAATTTAGTGGACAGATGATATGACAAGATGATAAGAGGGGATTTAAAAAAATCATCTAGCAAAGCCCAAGAGGA
AAAAAAACAAAGCTATTTTAGAAATGAAATACCAATTTGAAGCAGTAAGAATAGATTGGATATCTTTGAAAACCATTAATTGAATGA
AGAACCAATTTGAGAAAACAATACAGAATGCAAAGTAGAAAGATACAGAAATAAAGGCAAAAATTATAATATGGAAATCAGACAATG
GATTTGTCTGTATCCAGTTATGTGGATAATTAAAATGGAGACCCTCAGAAAATTGAACCGAAGAGTAAAATGAAACTCAAAAATGTA
GTAGAAATTGTTGGGAAGTAAAGAAAACTTGAATATGTAGATCAGAACATATATGTTGATGACGTTATTGACTTTGAGGTTAAAAAT
ATATATATGTGCCTATGATTATGGGGAAAAAAGCAGTCGTCTCAGAAAGAAAAACATCAAGTTAGTCTTAGACTTTGCAGTGCACTC
AGTACCAAAGAGAGAGGAGGTCCAGACTTGGACCTGCGAGGGAAGAATAATAACCGAAAATTTTATATCAATTCAAAAAGACATTGT
CAAAAATACAGGGATTCAGGAAACTGAGAATGCACTAAGCCTTCTGGAAAAAACACCTAATGACAAAATCTAGCCCAACAAGATGTA
AATGAATATAAAGGACTCATAATGAGGAAACCGCATTATGACTGGCTCTCAACCCTGGCTGCATATTAGACTCGTCAAAGACCTTTG
TAAAAGGTCACACATTGACTCGTCAAAGCCCCTCTCCAGACTAATTCAATTCAGAATCTCACAGATGGGGCCACAGAATCAGTATTT
TTTGACACAACCTCAAGTGAGAATATTGTGTAGACAAGATTGGAAACCACTGATTTAGATATAGAAACAAAGGCTAATCAACTGTGA
GAATTATGGTCACAGAATAGAAAGTAACTATTATGAACACTGAAAATGTAAAAAAAATGTAACAAAGAAAAATAGTTAGAGGAAGGA
GAGGAAGTAAAGGAACAATCATTTTCTCATGATTATTATTATTTCAGAGTAAATTGTGAGTTATTTCACAATTCAAAAAGAATGGAC
TGTTTTAAAAAATTAGTAATAGATTTCAAAATGTCCATTTTGTAAATCGTTTCTGAATACTTTGTCAACAGTTACTCATCATTAATG
GCTTATACTTCACTAAAATTCCATGGAAAACCAACTAGTAGCCTGTAGAGTCACATAGGAGAGAACAAGTGAATTCTTTGGGTGGCG
CAAGCATAGATGTTAGGACTGACAAAAAAAAAATAATAAAAATAAACCTGTGCATTGATATGATCACAAATGATCAGGGAAAGAGGA
AACAGAAACTCTCATACGCCATTATTACAAGTGTAAATTGGTTCAACCTTTTCGTCTTAATTGACACATTGTAATTGTATATATTTA
TGGAAGCACAGTTTGATATTTTGATATACATACATGGTATATAACGATCAAATTAGGATATTTAATGTACCCATCATCTCATGCATT
TATCATTTCTTTGGAATAAAAACATTCAAAAGCCTCTCTTCTAGCTTTTTTGTAATATATTATAACTGACCATTAACCATCATCACC
AATAGAACAGAATTTATTCCTCCTGTCAAATTATAACTTTATACCCATTGACCAACTTCTCCCTATCCTCCTCTTCCCTCTCCCCTC
CCCAGTCTCTGATAACCACAGTTCTATTCTCTGCTTCTATAGTATCAACTTTGTATTTTTTTAGATTCAAAATATGAGTGAAATCAT
GCAGTATCAGTCTCTCTGTGCCTGGATAATTTCATTAAATATGATATCCTCCAGGTCTTTTTATATTGCTGCAAATCACAGGATTTC
ATTATTTTTTATGGCTGAATACTATTCCATTGTGCATATATTCCACATTTTATTTATCTATTCATCTGCTGGGGGACACTTAGATTG
CTTCTATATCTTGGCTATTGTGAACAGGGCTGCAATAAACTTGAGAGTGCAGATATCTCTTTAACATAGCGACTTTATTTCCTTTGA
ATATCCACCCAGCAGTGAAATTGATGGATAATATGGTAGCTCTATTTTTATTTTATTGAGAAACTTCCATGCTGTTTCTTGTGGTGG
CTGTACTAATTTACAATGCCACCAGCAATGTAAGGGTTCCTTCCTCTCCACAACTTAACCAGCACTTGTTTTCTTCTGACTTTTTGA
TAACAGTCATTCTAACTGGAGTGAGGTTATACTTCATTGGCGTTTTAATTGTATTTCCCCAATAATTAGTGATATTGACCATTTTTT
TCATGTACCTGTTGTCCATTTTTATGTCTTCTTTAGAGAAATGTCTGTTAAAGTCTTTTGCCCATTTTTTAATCAGCTTGTTTGGTT
TTTTCCTGTTAAGCTCTTTATATATTCTGGATGTTAACACCTTGTCAGACATAAACTTTGCAAATATTTTCTCCCATTCTATAAGTT
GTCTTTTTACTCTGTTAATTGTTTCCTTTGCTGTGCAAATTCTTTTCAGCTTGATGTAATCCCATTTGTCTAATATTTGCTTTTGTT
GCCTGTACTTTTGTATTTTAAAAGGTCCAGTCTTTTAGAAGAACAATTTGACATTATATGTTATTACTTAAAATGTTTGTATACTTC
ATCCCAGAAATTCCATGTCTAAGAATTTATCCTAAGAAATGCATGTGTAAATATACAAAAATATATCTACAGAATTACTTAATGCAG
CATCACTTGACATTTAAGAAACAACAACATCCCAAACATCCATTAATGGGAAAATGCTTAAATGTATTATAGCATATCTATACAATG
GAATACTTTGTCATTGTTAAGAATAATCAGGTAAGTGGGGCATGGTGGCTCTCGCCTGTAATCCCAGCACTTTGGGAGGCCGAGGCG
GGCAGATCACCTGAGGTTGGGAGTTCAAGACCAGCCTGATGGAGAAACCCTGTCTCTACTGAAAATACAAAATTAGCCGGGTATGGT
CGTGCATGCCTGTAATCCCAGCTATTCAGGAGGCTGAGGCAGGAGAATCGCTTCAACCCGGATGGCGGAGGTTGTGCTGAGCCAAGA
TCGCGCCATTGCACTCCAGCCTGGGCAACAAGAGTGAAACTCTGACTCAAAATAAAATAAAATAAAATCAGGTAATATATTTCCAAA
ATTATCAAGTTGTAAATGTAAAAGTTATACTTTATTGTGTGTAAATTATGCTTTAATAAAGATGAGTTTTAAAAGTTAGTGATGTAA
ATTTTTAAGTAATACCACAGAAAGATGCCCATAATATATAGCTAAGGGGAAAAAGCAGGTTGTAGACCAGTATGTTATTGTGATCCC
ATGCACGTGTGTGATTTTAATGTATTTATTTATGTGTCTATGAGTGTACTTATACATAGAAAAGATCTAGAAAGTGTCACCAAATAC
TCAACAGAAGTAATTTCTGAAAAAATAAAGAAAATGGTAAAACTTTTTATTTTTACTTAGATAATTCTGTAAAGTTTGATTTTATTA
AATGAATAGCTGGGTTTTTCATTTTTAAAAAAATTTCAGTAGTTTTGGGGACCAGGTGGTGTTTGGTTACATGAGTAACTTCTTTAG
CAGTAATTTCCGAGATTTTGGTGCACTCGTTGCCAGAGCAGTGTACACTGAACCCAGTGTGTAGTACTTTATCCATCACCCCACTCT
CACTCTTCCACTGAATCCCCAGAATCTATTATATTATGCTTAGGCCTTTGCGTCCTCATAGCTTAGCTCCCACTGATAAGTGAGAAC
ATACAATGTTTGGTTTTCCATTCCTGAGTTACTTAGAATAATGGTCTCCAACTCCATCTAGGTTGCTGTGAATGCCATTATTTTGTT
CCTTTTTATGGCTGAGTAGTATTCCATAGTGTACACACACACACACACACGTGCGTATGTATCACATTTTCTTTATCCACCCCTTAA
TTGATGGGCATTTGGGCTGGTTCCATAGTTTTGCAATCATGAGTTGTGCTGCTATAAACACGTTTGTGCAAGTGTCTTTTTCATATA
ATGACTTCTTTTCCTCTGGGTAGATACCCAGTAGTGGGATTGCTGGATGAAATGGTAGTTCTACTTTTAGTTCTTTAAGGAATCTCC
ATACTGTTTTCATAGTGGTTGTAATAGTTTACATTCCCACCAGCAGTGTAAAACTGTTCCCTTTACACCACATTCATGCCAACATCT
ATTATTTTTTGTTTTTTTAATTATGACCATTCTTGCAGGAGTAAGGTGGTATCGCATTGTGGTTTTGATTTGAATTTCCTTGATAGT
GATGAGCACGTTTACATGTTTGCTGGCAATTTGTGTATCTTCTTTTGAAAATATTCTATTCATGTCCTTTGCCCACTTTTTGATGTG
ATTGTTTTTTCCTGATGATTTGTTTGAGTACTTTGTTGATTCTGGATACTGGTCCCATGTCAGATGCATAGTCTGTGAATATTTTCT
CCCACTCTGTGGGTTGTCTGTTTACTCTGCTGATTATTTCTTTTGCTGTGCAGAAGCTTTTAAATTTAATTCAGACCCATCTATTTA
TTATTGTTTTTGTTGCATTTGCTTTGGGTTCTTGGTCATGAACTCTGCCTAAGCCAACCAATGTCTAGAAGAGTTTTTTAAAGTATA
TTCCAGGGTTTATATGGTTTTAGGTCTCAGATTTATGTCTTTGATCCATCTTGAGTTGATTTTTGTGTAAGGTGAGAGATGAGGATC
CAGTTTCATTCTTCTACATGTGGCTTGTCAATGATGCCAGCACCATTTGTTGCATAGGGTGTTCTTTCCTCACTTTACATTTTTGTT
TCCTTTATCAAAGATCAGTTGGCTGTAAGTATTTGGCTTTATTTCTGTGTTCTCTCCTTCCATTGGTCTACCTGTCTGTTTTTATAC
CAGTACCATGCTGTTCTGGTAACTATAGCCTTGTAGTATCGTTCAAAGTCAGGTAATGTGATGCCTCCAGATTTGTTCTTTTTGCTT
AGTCTTGCTTTGGCTATGTAGGCTCTTTTTTTTGTTCCATATCAATATTAGGATTGTTTTTTCTAGTTCTGTGAAGAATTATGGTGG
TATTTTGATGGGAATTGCATTGAAGTTATAGATTGCTTTTGGTAGATGGCCATTTTCACAATATTGATTCTACCCATCCATGAGAAT
GGGATGTGTTTCCATTTGATTGTGTCATCTGCGATTTCTTACACCAGTGTTTTGTAGTTTTTCTTATAGATATCTTTCACTTACTTG
TGAAGGTGTATTTCTAAGTATTTTATTTTTGTAGGTGTCATAAACAAGTTTGAATTTTTTATTTGATTCTCAGCTTGGTCACTATTG
GTGTATAGCAGTGTTACTGATTTGTGTACATTGATTTTGTATCCTGAAACTTTACTGAACTCATTTATCAGATCTAGGAGCTTTTTG
GATGAGTCTTTACGGTTTCCTAGGTATACAATCATGTCATCAGTGAACAGCAACAATTTGACTTCTTCCTCTTTACCAATTTGGATG
TCCTTTATTTCTTTCTCTTGTCTGATTGTTCTGGCTAGGACAAAGAGCTGTTTCTAATTTTAAAAATACATGTGTCCCTAACTCATT
TTCTCTCTCAGTTCCAGTGTCTCGCCCCATCCTCACCCTCAGGGTTCCCAGGGCCCAGGCTGTGGTGGGCGACCTGCTGGAGCTTCA
CTGTGAGGCCCCGAGAGGCTCTCCCCCAATCCTGTACTGGTTTTATCATGAGGATGTCACCCTGGGGAGCAGCTCAGCCCCCTCTCG
AGGAGAAGCTTCTTTCAACCTCTCTCTGACTGCAGAACATTCTGGAAACTACTCATGTGAGGCCAACAATGGCCTAGTGGCCCAGCA
CAGTGACACAATATCACTCAGTGTTATAGGTAAGTTGACCTAACCAGCAGCACAAAAATAAAAAGACCATCTCCCCTAACTTTACCA
GTAGCCAAGATCAGAGGTACAGTTTCTAGAGTCATTTGCTGTGTGACATCCTGTCAGGCTTGGTGCCTACACAGCCTCCATCTCTGA
GTTTTGTATCCTACACGTTCAGGAACTTGCACCTTCCCTTCCCTTGCTGTACTTTTCCTGCTAGCAGCAGTCCAGCCATCACGACCA
AAATTCCCCTAACATAGGAGTAAAGGCTTTCAAAATACAAACTATATTCTGGCCAACAGTGATAGTAAGAAAGAAAGTTATAAAGTT
CTGTGTGCTTCACCTTGGAAGAGATTTAGTTGTGGTTTTCTCTGTCTGCCAATCAATAAAAAGGCCAACACAATTCTCTTATTGGCT
CACACATTTCTCTCTAGCCTCTCCTGCCCCTGTCCCAGAGTGGGTTGGGTTCCCACCCAAGACCCTTGTTAGAAGACATAATTCTTC
TGCCTCCTTACCTGCTCCTGTTCCATTCAGCAACATCAAGATCTTCCATGCATCCTTGTCACAGGCAGACTCCTAGTTATGCCCACT
CTCAGTGAAGGCTTTGGCACTGTACTTTCAGTCTTTCTCCATACTCCATAATCTGCCATCATTTCATGACTCTCATGGTCACATGTA
TAATCCCCTTAACACCATGGCCTGTCCTCTCCACACCAGTCAGCCACTCCCACATACATACACTACTTCATCTCCAAAAGGACAATT
TTAAGCCCTTATTATCTGACAACCACCCCCTGGCCTTCTGCTTGCGTCAGCTCCTCCTGTCACTATTGTTCTCAGACCTCAACAGAA
CTCCCAGGTCATTAATACCTCCACTAATTCCCTACTCCACTAGACAATAACCCCCTCCACTCCCACCACCAAATTGAGTGTCTAGCT
CTGTATCCACCCAATAACTCATCTGTCCTTTTCCTAAAATATAGAGGAGAGGACTTCCCTCCCCATAGGTCAACATCTCCATCTGGT
CTCCAGATTTCATCATTTTGTGCATTCTCAGAAGCTATGTAACTGATTATCCCTTTCTCTTCTGTGGCCCCCAACATCTCTGTCTCT
GTAGGATCCTTTGCACTCGCACGTAACATGGTATATCTCTCCCATATCCAAGGAAAACCCTCTCTTGGTCCCACTTATACCTCCATA
CACTACCCCTTCATGGCTAAACTTCCAAAAACAGTCAGTTACCCAGACTGTCTCCATGTCCTCACTATCCACTAGGCTCTAATCTAC
TAGGTTTTTCCAACCTCTCCACCAAAATGTTTCTCACAAAGGTCACCGATGACCTTCAAGGCTTTAAATCCAATGGCCATTTTTCAA
TCCTCATCTTCCTTGACATCTCAGATATATTACATGTTGTTTACCATTTCTTCCTTCTTGAAACCCTCCCTTCAGTGAATCTATCTT
CTTCTGGCTCTCTTCTCTCCCTGGCCACTTCTTCTCAGTCTTCTTTACTGGCTCCTCCTTTTATAGAAAACCTTTAAGCACTGGCAT
GTTCCAGAACTTTGACTTATGCCCTCTTCTCTTATCTAAATCCTTTGTCTGGATGATCCTGGGTATGTCATGTATAAATGGCCTTGT
CATCTATAAATAATGATTCCTAAACTTCTATCTCTAAAAGGGACCTCCCACTTGAGCATCTGCACATGACATACCTTCACTCAAATA
TCTCAAAAGCACATCAATCTGAGCATTTCTAAAACTGAACTGCTGACATTCCCTTCCTCTTTCAAGGGTTCCTACCTCTGTGAAACA
CATCTCTTCTCAACAGATACACAAAGCAGGAACAGAGGAGTCTTCCTCAACATTTCTCTTCCTCACCCCTCACAGCCAGTTAATCAT
TAAGTCCTGTTGATTTGACTTCCTAAATATTTCTTGGGTCTCTATCTCTACCACCTCTATCTAGACAAGTCCACCATCATTTTGACT
ACTGCAGTGCCACTCATCTATCCCTCAATTCCACTTCCATTCCACCACAGTCCATTTCATGCACAGAGGTCAAAATGGTCTTTTAAA
AGGGACAAATTTGATCATCTCACAGCCCTGCATAGATAGACTTATAGACTTCCCATTGCTCTGTGAACAAGGATCAAAATCCTTTCA
TGGCTTATGAAGTCCAGCCTAGATCTCCAGTCTCCTATTTCTTGGTAGTTTGCAGAATCTTCTCAGTGTGATGCTACTTTATTTCAA
TAATTCAAATGTGCCAGTCTTATTCCCAACTCAGGGCGTTTGCACATACTGTTTGCCTGCCTGGTGCACTCTCCACCTCCATCATCA
ACTGGTTAATTTCAATGTTAATTTCAAAGATCATTTTGTGAGGAAGCTTTCCTTGACTAACACATACATTTCATAGTACCCTGTCCT
TTATCAATTATCAAAGTTTTAATTGTAATTTACTGTATTTTTATTTAATTGCTGCCTCTCATCCTTCTAGATTGTAGGCTCTATGAG
AGCAGTAACTATGTCTTTCTCATCACTGATTCCCTAGCATGTCATGCCATTCTTGGGACTTATTAAATGCTCAATTAATTTTTATTT
TTGCTCAATTTTTAAAAAAATTTTTGTTCAATTAATTTTTGATGGATAAATGAATGTAAAAATGAGCAAATAACTTGACAATACTCA
TTCTCTTGTGGGCCCTATTTCTGATGCAGCCCAGATGCAATTACATCCCTGGTGATGTAAGCAGCAAGTCTCTCCTGTTCTTCATTT
TCTGTTGAACTGATTTCTCCTCTCTCCAACTGTCTTTTTACCTCTAAGACACCTGAGTTTCAGTTTAATCTTTGTTTTCACATTCTT
CAAACACATTTGAGTTTGTATATTCCACTATTCCAAGCTGTTTATAGCACTACACAAATGTTGTTCTTTCTGGAGATCTGAAAATAT
TGCTGAGTTTTGAGTAATGTCACAAGGGCATTAAAAACTGAGAAAAACATTCTTCCATTTGAAATGTCTTTAGCATGAAGGAATATA
TTTTAGCAGAAGCAGCTGATACATAACGTTTTTCCTCCCTCAGTTCCAGTATCTCGTCCCATCCTCACCTTCAGGGCTCCCAGGGCC
CAGGCTGTGGTGGGGGACCTGCTGGAGCTTCACTGTGAGGCCCTGAGAGGCTCCTCCCCAATCCTGTACTGGTTTTATCATGAAGAT
GTCACCCTGGGTAAGATCTCAGCCCCCTCTGGAGGAGGGGCCTCCTTCAACCTCTCTCTGACTACAGAACATTCTGGAATCTACTCC
TGTGAGGCAGACAATGGTCTGGAGGCCCAGCGCAGTGAGATGGTGACACTGAAAGTTGCAGGTGAGTGGGCCCTGCCCACCAGCAGC
ACATCTGAGAACTGACTGTGCCTGTTCTCCCTGCAGCTGAAAATGGAGCCACAGAGCTCCTCAGGGCTGTTTGCTTGTGTGGCATCC
CAGCACACTTCCTGCCTGCAGAACCTCCCTGTGAAAGTCTCGGATCCTTTGTGGTATGGTTCCAGGAATCTGATGTTTCCCAGCAGT
CTTCTTGAAGATGATCAAAGCACCTCACTAAAAATGCAAATAAGACTTTTTTAGAACATAAACTATATTCTGAACTGAAATTATTAC
ATGAAAATGAAACCAAAGAATTCTGAGCATATGTTTCTCTGCCGTAGAAAGGATTAAGCTGTTTCTTGTCCGGATTCTTCTCTCATT
GACTTCTAAGAAGCCTCTACTCTTGAGTCTCTTTCATTACTGGGGATGTAAATGTTCCTTACATTTCCACATTAAAAATCCTATGTT
AACATAAGCTATGTGTACAGCCCTTCAGGAGACACACTCAGCGATCTTTAAAGCAAGGGACACAGGTCACCAGGGCAGAGAGTGGGG
AGCAGGATGACATGGAATGGAAATGGACATCCATCACCCAAATTCCTCTATTCTCGAAGCTGTTCTTTGATCTCTTTCAATTAAGTG
TAATAACCCAAGGAAAGCTGCTTCTAGTGAGCTCTGAATCTTTTTGGTGATGACCTCCTACAAGCCAAACTTTGAAACCTCTGTTTA
GAGAGAACTGGTGTTTAGCAAAAGTATGCTAAGGGGTGTTGAGATGAATCTTCTAGACTTGGATCTGAAGATAACTAGCTGTATGAC
ATTGGCCAAGCACCTCTTCCCATACTGACCTCAATTTCCACACCCACAAAGTGGGAAAATCAGACTAGATGAAAATCATGATCATCC
TTTGTCCTAGAATTCTATGATTTTGGTAGACAGGGAATATGGATTATGCAGACGTACAGGTTCCTAGAAGGAGATATGCAAAAGTAA
AAGCAGGTTGTACCTGCCCCTGGAATATATCCTGGAGAAACACGAGACCATACAATGGTATGCAATCACCATTCCAGTCATGTAAAG
CAAAATATACACTGATGCTCTTATTCCCTTTGGCAAGGTGCCTGGGTATCATAAGGAATAGACAGAGTCACATAAGCAGGTTGATCT
GGGCATGCTAACCCATGATCCAGCATCTCACAAAGTAGCCAGTGCAACCATCTCATTGTTATCCAGGAGGTGATGACTTCAGATTTG
GGTTTTCCCATTCAGTCAGCTCGTTCTTCTTCCCCCACCAGCAGCACCAACCTCATCCAATTTTCCTGAGATTTGCAGGGGCTCTCC
CCACACAGTTTACACACCTATCCCTTAGAGCCAGCACACAGGCATGTACCACGGACCGAGGGCTGCTCCATCTTTCCTCACAATACA
GCAAGCCAACACATCATACCTCCTACATTCTATACTTATGATTTCATGATTTTCTGCTCATCCTCACTCCCTCATTTCTATAATTCT
CTGTCGCAAATGCATGTTATTTCTGCTGCTGTTATCACAGCTAAAGGGTTTCTTGCCCAGCCTGAATGAATTGTGTCTGAATCCTGA
AACTAGGACCCAGGTCATAGAGACCAGGAGCTGCCAGAAAAGGAAATATTATTTCTTATTATCATCATAACCAGTGTCATCATCATC
AACACTCACTGAGCCTTCACCAGGTTCCAGGCTCTGTGCTAAATGTTTAATAGGCAACATCTCATTTAATTATTAAGATAAACCTGT
GAGGATAAATTTTAAGCCATCGTATCCCCTATTTGCGGAAAAGAGACTGAGGTTTAGAGAAGTGAAGTAACTAGCTTGCAGACACAC
AGCTAGCCAGGGTAAAGCCAGGTTGAAGCACAGTCCTCTCTAACTCTTCACAETAGTCTGATACATGATGACATTTTCTCATCCGTT
GTGGCCACTTTTAACACAACATTTTAGTTTTTAGTAATTTGTTATTCTGAAAGAACACATGAATGTTTGTGATGAAAAAAATCAAAT
AATACAAAAGGGCCAATATTGAAAAGTAAAGGTCTCTTTCTACCTGCTCATATCACTTCTTACGCACATATTCTCCAGACATAACCA
CTGTTAATTGTTTCATGTGTATGCTTCTAGAAATAATCTACAAACCTAAAGGTAGATAATTTTTAGAAAGCTAGAATAATATTACAC
ATATAGTTATGCCAGTTGCATTTTTTAACTTAAAATACATCCTGACTTTTGTTTAAGAAATGCATCTTGGATATCATTCATGTTTCA
TTACCCAATCCAGTAGGTTGTCTCTAATTTTTAGCTCTTGTAAAAATGCTACAATAGAAATTCTTGTAGAAATATATTGGCATATTT
TATGTCAGTATCCCTAAAGGAGATGGCCCTAGAAGTAGGACTTGAACTCCTGGGCTCAAGTAATCCTCCTGCCTCATACTCCCAAAT
CGCTGGGATTATAGGTGTGAGCCACTACACCTGGCCCACTAGAAGCAGATTTGTTGGGTCAAAGTTTATGTGCACTTTAAATTTTGT
AGATATTGCCAAGTTGCCATTCAAGGAAGTGTGTTAAATTTGTATTACCACCAAGGGCATATGTAGGTGCCTGTTTATCTACAAGCT
AATCACTATGATCAGTCTATTTAAGCTTTGCCAACCTGAAAAATAGAAAAAAGAATAGTATTGTATGGTTGTCTTTGTGTTTAGTTA
ATTAGTAATGAAGATGAGAATCTTTGTATATATTTATTGTCTATTTGAAGTTTTCAGTGATTTATACACTTTTTACAGTTTTCTATT
GGATATTTTTGTACACTGAAAACTATTAGCAGGTCAAAGTAATTAGCTTTATCATCTATGTTTTAATCTACTTTCCAAGATTGAGTT
ATGACTTTTTACATTTGTTTATGATACCTTTTGTTTCTTACAAATATGTTAGAAGCAGAAACTGCTGTTGACTTTTTGTTCTCTTTC
AGTTCCGGTGTCTCGCCCGGTCCTCACCCTCAGGGCTCCCGGGACCCATGCTGCGGTGGGGGACCTGCTGGAGCTTCACTGTGAGGC
CCTGAGAGGCTCTCCCCTGATCCTGTACCGGTTTTTTCATGAGGATGTCACCCTAGGAAATAGGTCGTCCCCCTCTGGAGGAGCGTC
CTTAAACCTCTCTCTGACTGCAGAGCACTCTGGAAACTACTCCTGTGAGGCCGACAATGGCCTCGGGGCCCAGCGCAGTGAGACAGT
GACACTTTATATCACAGGTAAGTTTCCCGTGCCCGGGCCACAGATGCGGACAAAATGTTTCCTCCGCGGACCCTCTGATAACCCTCA
CAGTGCTTGATCAGAAAGGCACTCCTCAGGGCGAAGTAACACAGCTGGCAGCTCTCTAAACTGCTATATTTCCTGAAGTGCTTCTGA
AGTGATGGCGTTGGGGCTGAGAGCGCGGGTTGGCGGGCTGGGCGCAAGAGCGCCACTGACTTTAGAGGTCCTCACCTGGCTGCCTCC
TCTCTCACTGGCCACCGGCCACCCTGACCCTGGACACTTGTCTCAGGCGCAGAGACGGCTTCCTCACAAAGACAGGAGCCATCTTCT
GTGCCACACCAGCCCCTGCTTAGCTGCCGGGGACCTGCTCACCTAGGTTCACCGTCAGGCCCAGGCAGCAGCTTCCGGGAGCGGGCA
GTTCCAGACCACAGCCTCTCCTCTGGCTCACACACAGGGTCTCCCCCTCAGGGCTGACCGCGAACAGAAGTGGCCCTTTTGCCACAG
GAGTCGCCGGGGGCCTGCTCAGCATAGCAGGCCTTGCTGCGGGGGCACTGCTGCTCTACTGCTGGCTCTCGAGAAAAGCAGGTGGGT
GACTCTGGCCACACCTTGGCTTGAGTTCATGCCCTTCCCAGCAACGCTCCCTTGGGCTTCATGCTGCCTTTGTGACCATCCCAGGCT
CGCAAGAGCTCATCCGCTCTGTCCCTCAATCCTCTTCCTTTCCCGCTGGGTCCTCCCGAATTGCTTTTACCAAATGCACAGTTTCCA
AAACAGACCTATAAGGTGGGAGACGTCTTGATGAGATGTAGTTTATCCAACATCATAAACTACAGAGTGTCTTCCTCTCGACCCTTA
TAAGACCCCCCCAACGAATGGGAAGAGGAGGAGAAAAACTCAGAGAAATAGAAATGAAAATAGAAACCAGATAGGATTCAGTAATTT
TCCGATTTTTTAAATGTACACTATTTTTATAGTTGATCAAGTCTGTTTTCCTGCCTTGGAGATTATCTTTAAAATCTCCAGGTTAAA
GGATTACGGATTGTGGACTCCCGTTTCACATCATAGCAAACCCCTCTGGCAGGGTCAGTAGTTTTTGCCCAATCTGCTACCTCCCTG
GGGCATAACAAAGAAGGATTCAACCTTCAGTGACAGCAACTCCTGGCAGTTTCTAAGTGAGCCTTGCCTGAGCTGTGCCCCACTGAG
GGTCTCATCTTCCTGAATCCAATGCTCCTCTGCTTTCCTTTACAGGGAGAAAGCCTGCCTCTGACCCCGCCAGGTAAGAGCTGAGCT
GCATTCCTGTGTGATTAGTGAAATGTCCACAGGGTGGAAAAATGGAATTGAATCTGATGATTACCAAGAGCACAGTTTCTGCCAGGT
GCTGCTGAGCTGGGGTGTGGCCACTACCTGTTGTACGTCCCTGGGGATATCTTAAGGCAATTCCCAGGACCTTTGCACCCACCCCCT
TAGCTGTATAGGCTGTTATCTCATCAAGTATTAATTGTCTTTTCATTTATTAACCCACATCCTTGGACCATTGTTGGCTTGGTTGTC
TGAATGACCACTATGACCACCAAGTCTTTCCATATCTTGAGATGACCTTAGCTCAGTCAACCTAGAAATCTCTGGAGATGCTGATCT
CATCAACACCTCTTGTGATAACAACTGAGAAGATGGAGCCGCACAACTTGGCCACCCAGAGAGGTCACTCTGCCAGCAGGAGGTAGT
CCCTGTGAAGAAGGGGCCACCATCTTGTATCCCTAAGGCTGACTTTAGGAGCCACACAAAGGCTCACAGCTATCCCTGGGCAAGCCC
ACGATAGCAGCATCCAAACATGTAGAGCACCTCGGAGCCTTCTGGTCACTGATCTCTCTGAGCATGCAGGGTTCGGAGCCAGCTTTG
AGCCTTGGCTCATCCCCTTTCTGGTTACATGATCTTCATAAGTCACTTATTGGAGCCTCCGTTTGTTTCAGCTAAAACGAGGATGAA
ATAGAACTAACAACGTATTGCTGTAAGCATCAGCACCATGCCTGTTACACACTAGATGACCAGAAAGCCTGGAAACATATATAATAT
GATTATGATTCATAATAGAATGCCCATGGTCAGTTTGTTATGTACTTGTTCATGTTTCCAGGCCTGGCAGCCACCCTGCAGTAAGAA
GTCACTCACTAGGAGTGATCATTATAGCTACTTTGTCTGGATTCCTAAGAAACATTCACATTTCTCTGTCTCACAGGAGCCCTTCAG
ACTCGGACTCCCAAGAGCCCACCTATCACAATGTACCAGCCTGGGAAGAGCTGCAACCAGTGTACACTAATGGTGAGGCCCGAGGAT
CCTGCATTTGGCAGAAGCTGGAGGGCTGGTGTGGGAAAGCGGTGGGGCAGGAGATGGGGAGGGACTATGCCGAGGCCTAGAGGGCTC
TAGTCCTTCCAGAAAAGAGACTCTGGCTGGGGGAGGAGGAAGTATCTCTAACAAGAGGCAAGAACCTCAGCATCCTCTGAAACTTCA
ACTTTTTTCTAAGCAAATCCTAGAGGAGAAAATGTGGTTTACTCAGAAGTACGGATCATCCAAGAGAAAAAGAAACATGCAGGTAAG
ACCCAAGGATGTCTCTGTTCTGGCCCACCCTCAACTTAGACAGTATAGCTCAGTTTCTACCCACTCAGCAGATCTGAGCCAGACAGG
CATAGAGCCGCCCCTCCCACTGGGTCATGGGGCTTCTTTATGTGTGCCTATGCTGCTTCAGGTGAATCCAAGCCTCCCCTGCACTCT
CCTTTCCCTATTCTCTGCAGAGGGCCCCAGTATAACCTGAATAACAAAACTATCTTTTTGGGCATTCGCTTAACCTCTCAGACCTTC
CATATCCCTATCCATAAACTGTGACTACTTCCCTACTGTCTTAGTCAGTTCTGGCTCCTGTAACAGAGTACCATAAACTAGGTGACT
TAAAGAACTAATATCTATTTCTCACAATTCTGGAGGCTGGAAATCCAAGATCAAGGTGCCAGCATGGTCAGGTTCTTAGTGAGGGCC
CTATTCCCATTTTCCAGACAGATGACTCCTTGCTGTATCCCCCACACAGCAGAGAGAGAGGGAGAGATCATCTTTCATGTCTCATCA
TGATAATCATCATGACATAATTGTCACCCAAAGGCTCCACCTCCAAATGCCATCACATTGGGGATTAGGTTTTAACCCATACATTTG
GAGAGCAACACAAATATTTAGTTGATAGCACCTACCTACCTGTACTAGTTTCTGATTGCTGCTGTGACAAATGACCATAAATTTGGG
GGGATGAAGCAGTACAAATTTATCATCTTACAGCTCTGTGGGTCAGAAGTCTGACGCCGGTCTCACTGGGCTAAAATCATGGTGTTG
GCAGGGCTGTGTTCCTCCCTGGAGGCCCATCCACTTTCCTGCACCCATGTCCCCTTCCTTTCCCAAAGCCAGCAAAGTCACATTTCT
CTAATCATTCTTCCATAGACAGGCCTCCTTCTGACCTCACCTGAGAAAAGTCCTTGGGTTTTAGAGAATCATATGAGTATATTGGGC
CCATCTGGATAACCCAGAATAATCTCCCTATCTCAGGGTCTGTAATCCTAATCACATCTGAAAGGTCCTCTTTTCCACTTAGGGTAA
TATAGTCACAGGTTGCAGCAATTAGGGCGTGACATCTTTGGGGGCACATTATTACAGCTACCATGCTGTTTCATGGGGTAACTGTGG
GGACTAAATGAGAAAATAGGTGTGGAAATGCTGTCGAAACTGCAGAGGGTTGACCAGCTGTGACGATGACTACATGCTGGAGGTCTG
GCTATGTCATCAACCCTCACTCACAGTCGGTGGTTTGAAATGAGGGAATCCCAGCCCGGCTGCCTCCTTACTCGGAGCAGGAAAGAG
GAACAAAGTCCTTCACGGCAGGCTTTTCTTTCTACCTCTGCTTCATAAAAAGTGCTGCGTGATTTTAGCCAGTGTGCTTCCCTCTCT
GGTTTTCTAATTTTTCTTATATAAAGTCAAGTGAATTGTGTTTGTGCCTCATTCTTGGTTCTATAGCACCCCTCTGAGACATGTGAA
CTCATTCACTCTTACAAGCCCCTTTGAGGTATGCGCTGTTACTACTCCCATTTTACAGATGAGGAAACCAAGGCATAGACACCTTAA
ATGACTTGCCTGAAATCTTATAGTCAATCACCAAACAGTCTTCTCCAGAGTCCAAACTCTAGACCAAACTCTGCTTCTTCATGCAAT
TCCAGGGAGTCCTGATGCTCATTCCAAGCCAAAAGGGCAGAGCCTTGTCTGGAGAATGTCATATTTCTCTCCAGTTTGATGATAATA
TAACAATAATAATTTATAATATCTATCATGTGCTGAATAGTTCCTATTATGTGCTGGGTATAGTTCTATAAGTGCGATAAATAAATA
CACTATATTTGTATGGATAGCTCTTAGAACTATACCTGTAGCACTTATATAGAACTATGGCATGCATACATATGATAATAGAGAGAG
AGAAAATGTATTGAATATAGCAATCAGGACTGTACCTGACACATAGAAACTGCTCAATACATGACAGATGATAGAAATAATATATGC
ACAGACACACAGATACATGTGCTCACACATATGTGTAAACTCATATAGCTTCACCACAACCTTACAGGCAGAGGATATTTTTATTCC
CATCATATAGATGAGGCAACTGAGGCAAGAGATGTTGAGTGACTTCCCCCAACCACGGAGGTGACAAGACAGGCGGCAGGCTCAGTT
TCAGGTGGTCTAGACCCTGATGGAGTGTGCTCTCTAAAACTTGCACCCACCACACCTTCCCAGAACAATAATACCAATTATGCTCAA
ATAACTAACAAGATAAAAGACAAATGACTTTCTCTTGAGGGCCTCTAGGCAGCTCAGAGTGGGCCACAAAGTGTGAAGGGGAGTTGA
AGAGAGGGTCTGTCAGTGCTGAGAGGCTGAGGGAGTTGGGAATGGAGAGGGACTGGGTGAGCACCAACTAAGAGGAATGGCTACTCA
CCGCACAGAGGGAGAGCGGGCCTCCAGGAGGCTCAGGGCTGGCCCCACAGCGCAGGCGCAAGGGAAGAGCCCCATGCCAGTCTTCCA
CAACTGAACACTTTCCTCTTTCCACAGTGGCCTCTGACCCCAGGCATCTCAGGAACAAGGTGAGTCTCCCCTTCTGCATGCCCTCCC
CCACCCCCAGGCCCACCCTGGTCCTCACGCATGTGCTCTTGCCTCCTAGGGTTCCCCTATCATCTACTCTGAAGTTAAGGTGGCGTC
AACCCCGGTTTCCGGATCCCTGTTCTTGGCTTCCTCAGCTCCTCACAGATGAGTCCACACGTCTCTCCAACTGCTGTTTCAGCCTCT
GCACCCCAAAGTTCCCCTTGGGGGAGAAGCAGCATTGAAGTGGGAAGATTTAGGCTGCCCCAGACCATATCTACTGGCCTTTGTTTC
ACATGTCCTCATTCTCAGTCTGACCAGAATGCAGGGCCCTGCTGGACTGTCACCTGTTTCCCAGTTAAAGCCCTGACTGGCAGGTTT
TTTAATCCAGTGGCAAGGTGCTCCCACTCCAGGGCCCAGCACATCTCCTGGATTCCTTAGTGGGCTTCAGCTGTGGTTGCTGTTCTG
AGTACTGCTCTCATCACACCCCCACAGAGGGGGTCTTACCACACAAAGGGAGAGTGGGCCTTCAGGAGATGCCGGGCTGGCCTAACA
GCTCAGGTGCTCCTAAACTCCGACACAGAGTTCCTGCTTTGGGTGGATGCATTTCTCAATTGTCATCAGCCTGGTGGGGCTACTGCA
GTGTGCTGCCAAATGGGACAGCACACAGCCTGTGCACATGGGACATGTGATGGGTCTCCCCACGGGGGCTGCATTTCACACTCCTCC
ACCTGTCTCAAACTCTAAGGTCGGCACTTGACACCAAGGTAACTTCTCTCCTGCTCATGTGTCAGTGTCTACCTGCCCAAGTAAGTG
GCTTTCATACACCAAGTCCCAAGTTCTTCCCATCCTAACAGAAGTAACCCAGCAAGTCAAGGCCAGGAGGACCAGGGGTGCAGACAG
AACACATACTGGAACACAGGAGGTGCTCAATTACTATTTGACTGACTGACTGAATGAATGAATGAATGAGGAAGAAAACTGTGGGTA
ATCAAACTGGCATAAAATCCAGTGCACTCCCTAGGAAATCCGGGAGGTATTCTGGCTTCCCTAAGAAACAATGGAAGAGAAGGAGCT
TCGATGAGGAAACTGTTCAGCAAGAGGAAGGGCTTCTCACACTTTCATGTGCTTGTGCATCACCTGAGGATCCTGTGAAAATACAGA
TACTGATTCAGTGGGTCTGCGTAGAGCCTGAGACTGCCATTCTAACATGTTCCCAGGGGATGCTGATGCTGCTGGCCCTGGGACTGC
ACTGCATGCATGTGAAGCCCTATAGGTCTCAGCAGAGGCCCATGGAGAGGGAATGTGTGGCTCTGGCTGCCCAGGGCCCAACTCGGT
TCACACGGATCGTGCTGCTCCCTGGCCAGCCTTTGGCCACAGCACCACCAGCTGCTGTTGCTGAGAGAGCTTCTTCTCTGTGACATG
TTGGCTTTCATCAGCCACCCTGGGAAGCGGAAAGTAGCTGCCACTATCTTTGTTTCCCCACCTCAGGCCTCACACTTTCCCATGAAA
AGGGTGAATGTATATAACCTGAGCCCTCTCCATTCAGAGTTGTTCTCCCATCTCTGAGCAATGGGATGTTCTGTTCCGCTTTTATGA
TATCCATCACATCTTATCTTGATCTTTGCTCCCAGTGGATTGTACAGTGATGACTTTTAAGCCCCACGGCCCTGAAATAAAATCCTT
CCAAGGGCATTGGAAGCTCACTCCACCTGAACCATGGCTTTTCATGCTTCCAAGTGTCAGGGCCTTGCCCAGATAGACAGGGCTGGC
TCTGCTGCCCCAACCTTTCAAGGAGGAAACCAGACACCTGAGACAGGAGCCTGTATGCAGCCCAGTGCAGCCTTGCAGAGGACAAGG
CTGGAGGCATTTGTCATCACTACAGATATGCAACTAAAATAGACGTGGAGCAAGAGAAATGCATTCCCACCGAGGCCGCTTTTTTAG
GCCTAGTTGAAAGTCAAGAAGGACAGCAGCAAGCATAGGCTCAGGATTAAAGAAAAAAATCTGCTCACAGTCTGTTCTGGAGGTCAC
ATCACCAACAAAGCTCACGCCCTATGCAGTTCTGAGAAGGTGGAGGCACCAGGCTCAAAAGAGGAAATTTAGAATTTCTCATTGGGA
GAGTAAGGTACCCCCATCCCAGAATGATAACTGCACAGTGGCAGAACAAACTCCACCCTAATGTGGGTGGACCCCGTCCAGTCTGTT
GAAGGCCTGAATGTAACAAAAGGGCTTATTCTTCCTCAAGTAAGGGGGAACTCCTGCTTTGGGCTGGGACATAAGTTTTTCTGCTTT
CAGACGCAAACTGAAAAATGGCTCTTCTTGGGTCTTGAGCTTGCTGGCATATGGACTGAAAGAAACTATGCTATTGGATCTCCTGGA
TCTCCAGCTTGCTGACTGCAGATCTTGAGATATGTCAGCCTCTACAGTCACAAGAGCTAATTCATTCTAATAAACCAATCTTTCTGT
A
HUMAN mRNA SEQUENCE:hR27-007.1 (Seq ID No: 17)
AATTCACTAATGCATTCTGCTCTTTTTGAGAGCACAGCTTCTCAGATGTGCTCCTTGGAGCTGGTGTGCAGTGTCCTGACTGTAAGA
TCAAGTCCAAACCTGTTTTGGAATTGAGGAAACTTCTCTTTTGATCTCAGCCCTTGGTGGTCCAGGTCTTCATGCTGCTGTGGGTGA
TATTACTGGTCCTGGCTCCTGTCAGTGGACAGTTTGCAAGGACACCCAGGCCCATTATTTTCCTCCAGCCTCCATGGACCACAGTCT
TCCAAGGAGAGAGAGTGACCCTCACTTGCAAGGGATTTCGCTTCTACTCACCACAGAAAACAAAATGGTACCATCGGTACCTTGGGA
AAGAAATACTAAGAGAAACCCCAGACAATATCCTTGAGGTTCAGGAATCTGGAGAGTACAGATGCCAGGCCCAGGGCTCCCCTCTCA
GTAGCCCTGTGCACTTGGATTTTTCTTCAGCTTCGCTGATCCTGCAAGCTCCACTTTCTCTGTTTGAAGGAGACTCTGTGGTTCTGA
GGTGCCGGGCAAAGGCGGAAGTAACACTGAATAATACTATTTACAAGAATGATAATGTCCTGGCATTCCTTAATAAAAGAACTGACT
TCCATATTCCTCATGCATGTCTCAAGGACAATGGTGCATATCGCTGTACTGGATATAAGGAAAGTTGTTGCCCTGTTTCTTCCAATA
CAGTCAAAATCCAAGTCCAAGAGCCATTTACACGTCCAGTGCTGAGAGCCAGCTCCTTCCAGCCCATCAGCGGGAACCCAGTGACCC
TGACCTGTGAGACCCAGCTCTCTCTAGAGAGGTCAGATGTCCCGCTCCGGTTCCGCTTCTTCAGAGATGACCAGACCCTGGGATTAG
GCTGGAGTCTCTCCCCGAATTTCCAGATTACTGCCATGTGGAGTAAAGATTCAGGGTTCTACTGGTGTAAGGCAGCAACAATGCCTC
ACAGCGTCATATCTGACAGCCCGAGATCCTGGATACAGGTGCAGATCCCTGCATCTCATCCTGTCCTCACTCTCAGCCCTGAAAAGG
CTCTGAATTTTGAGGGAACCAAGGTGACACTTCACTGTGAAACCCAGGAAGATTCTCTGCGCACTTTGTACAGGTTTTATCATGAGG
GTGTCCCCCTGAGGCACAAGTCAGTCCGCTGTGAAAGGGGAGCATCCATCAGCTTCTCACTGACTACAGAGAATTCAGGGAACTACT
ACTGCACAGCTGACAATGGCCTTGGCGCCAAGCCCAGTAAGGCTGTGAGCCTCTCAGTCACTGTTCCCGTGTCTCATCCTGTCCTCA
ACCTCAGCTCTCCTGAGGACCTGATTTTTGAGGGAGCCAAGGTGACACTTCACTGTGAAGCCCAGAGAGGTTCACTCCCCATCCTGT
ACCAGTTTCATCATGAGGATGCTGCCCTGGAGCGTAGGTCGGCCAACTCTGCAGGAGGAGTGGCCATCAGCTTCTCTCTGACTGCAG
AGCATTCAGGGAACTACTACTGCACAGCTGACAATGGCTTTGGCCCCCAGCGCAGTAAGGCGGTGAGCCTCTCCGTCACTGTCCCTG
TGTCTCATCCTGTCCTCACCCTCAGCTCTGCTGAGGCCCTGACTTTTGAAGGAGCCACTGTGACACTTCACTGTGAAGTCCAGAGAG
GTTCCCCACAAATCCTATACCAGTTTTATCATGAGGACATGCCCCTGTGGAGCAGCTCAACACCCTCTGTGGGAAGAGTGTCCTTCA
GCTTCTCTCTGACTGAAGGACATTCAGGGAATTACTACTGCACAGCTGACAATGGCTTTGGTCCCCAGCGCAGTGAAGTGGTGAGCC
TTTTTGTCACTGTTCCAGTGTCTCGCCCCATCCTCACCCTCAGGGTTCCCAGGGCCCAGGCTGTGGTGGGGGACCTGCTGCAGCTTC
ACTGTGAGGCCCCGAGAGGCTCTCCCCCAATCCTGTACTGGTTTTATCATGAGGATGTCACCCTGGGGAGCAGCTCAGCCCCCTCTG
GAGGAGAAGCTTCTTTCAACCTCTCTCTGACTGCAGAACATTCTGGAAACTACTCATGTGAGGCCAACAATGGCCTAGTGGCCCAGC
ACAGTGACACAATATCACTCAGTGTTATAGTTCCAGTATCTCGTCCCATCCTCACCTTCAGGGCTCCCAGGGCCCAGGCTGTGGTGG
GGGACCTGCTGGAGCTTCACTGTGAGGCCCTGAGAGGCTCCTCCCCAATCCTGTACTGGTTTTATCATGAAGATGTCACCCTGGGTA
AGATCTCAGCCCCCTCTGGAGGAGGGGCCTCCTTCAACCTCTCTCTGACTACAGAACATTCTGGAATCTACTCCTGTGAGGCAGACA
ATGGTCTGGAGGCCCAGCGCAGTGAGATGGTGACACTGAAAGTTGCAGGTGAGTGGGCCCTGCCCACCAGCAGCACATCTGAGAACT
GACTGTGCCTGTTCTCCCTGCAGCTGAAAATGGAGCCACAGAGCTCCTCAGGGCTGTTTGCTTGTGTGGCATCCCAGCACACTTCCT
GCCTGCAGAACCTCCCTGTGAAAGTCTCGGATCCTTTGTGGTATGGTTCCAGGAATCTGATGTTTCCCAGCAGTCTTCTTCAAGATG
ATCAAAGCACCTCACTAAAAATGCAAATAAGACTTTTTTAGAACATAAACTATATTCTGAACTGAAATTATTACATGAAAATGAAAC
CAAAGAATTCTGAGCATATGTTTCTCTGCCGTAGAAAGGATTAAGCTGTTTCTTGTCCGGATTCTTCTCTCATTGACTTCTAAGAAG
CCTCTACTCTTGAGTCTCTTTCATTACTCGGGATGTAAATGTTCCTTACATTTCCACATTAAAAATCCTATGTTAACA
HUMAN PROTEIN SEQUENCE:hP27-007.1 (Seq ID No: 18)
MLLWVILLVLAPVSGQFARTPRPIIFLQPPWTTVFQGERVTLTCKGFRFYSPQKTKWYHRYLGKEILRETPDNILEVQESGEYRCQA
QGSPLSSPVHLDFSSASLILQAPLSVFEGDSVVLRCRAKAEVTLNNTIYKNDNVLAFLNKRTDFHIPHACLKDNGAYRCTGYKESCC
PVSSNTVKIQVQEPFTRPVLRASSFQPISGNPVTLTCETQLSLERSDVPLRFRFFRDDQTLGLGWSLSPNFQITAMWSKDSGFYWCK
AATMPHSVISDSPRSWIQVQIPASHPVLTLSPEKALNFEGTKVTLHCETQEDSLRTLYRFYHEGVPLRHKSVRCERGASISFSLTTE
NSGNYYCTADNGLGAKPSKAVSLSVTVPVSHPVLNLSSPEDLIEEGAKVTLHCEAQRGSLPILYQFHHEDAALERRSANSAGGVAIS
FSLTAEHSGNYYCTADNGFGPQRSKAVSLSVTVPVSHPVLTLSSAEALTFEGATVTLHCEVQRGSPQILYQFYHEDMPLWSSSTPSV
GRVSFSFSLTEGHSGNYYCTADNGFGPQRSEVVSLFVTVPVSRPILTLRVPRAQAVVGDLLELHCEAPRGSPPILYWFYHEDVTLGS
SSAPSGGEASFNLSLTAEHSGNYSCEANNGLVAQHSDTISLSVIVPVSRPILTFRAPRAQAVVGDLLELHCEALRGSSPILYWFYHE
DVTLGKISAPSGGGASFNLSLTTEHSGIYSCEADNGLEAQRSEMVTLKVAGEWALPTSSTSEN*
HUMAN mRNA SEQUENCE:hR27-007.2 (Seq ID No: 19)
AATTCACTAATGCATTCTGCTCTTTTTGAGAGCACAGCTTCTCAGATGTGCTCCTTGGAGCTGGTGTGCAGTGTCCTGACTGTAAGA
TCAAGTCCAAACCTGTTTTGGAATTGAGGAAACTTCTCTTTTGATCTCAGCCCTTGGTGGTCCAGGTCTTCATGCTGCTGTGGGTGA
TATTACTGGTCCTGGCTCCTGTCAGTGGACAGTTTGCAAGGACACCCAGGCCCATTATTTTCCTCCAGCCTCCATGGACCACAGTCT
TCCAAGGAGAGAGAGTGACCCTCACTTGCAAGGGATTTCGCTTCTACTCACCACAGAAAACAAAATGGTACCATCGGTACCTTGGGA
AAGAAATACTAAGAGAAACCCCAGACAATATCCTTGAGGTTCAGGAATCTGGAGAGTACAGATGCCAGGCCCAGGGCTCCCCTCTCA
GTAGCCCTGTGCACTTGGATTTTTCTTCAGCTTCGCTGATCCTGCAAGCTCCACTTTCTGTGTTTGAAGGAGACTCTGTGGTTCTGA
GGTGCCGGGCAAAGGCGGAAGTAACACTGAATAATACTATTTACAAGAATGATAATGTCCTGGCATTCCTTAATAAAAGAACTGACT
TCCATATTCCTCATGCATGTCTCAAGCACAATGGTGCATATCGCTGTACTGGATATAAGGAAAGTTGTTGCCCTGTTTCTTCCAATA
CAGTCAAAATCCAAGTCCAAGAGCCATTTACACGTCCAGTGCTGAGAGCCAGCTCCTTCCAGCCCATCAGCGGGAACCCAGTGACCC
TGACCTGTGAGACCCAGCTCTCTCTAGAGAGGTCAGATGTCCCGCTCCGGTTCCGCTTCTTCAGAGATGACCAGACCCTGGGATTAG
GCTGGAGTCTCTCCCCGAATTTCCAGATTACTGCCATGTGGAGTAAAGATTCAGGGTTCTACTGGTGTAAGGCAGCAACAATGCCTC
ACAGCGTCATATCTGACAGCCCGAGATCCTGGATACAGGTGCAGATCCCTGCATCTCATCCTGTCCTCACTCTCAGCCCTGAAAAGG
CTCTGAATTTTGAGGGAACCAAGGTGACACTTCACTGTGAAACCCAGGAAGATTCTCTGCGCACTTTGTACAGGTTTTATCATGAGG
GTGTCCCCCTGAGGCACAAGTCAGTCCGCTGTGAAAGGGGAGCATCCATCAGCTTCTCACTGACTACAGAGAATTCAGGGAACTACT
ACTGCACAGCTGACAATGGCCTTGGCGCCAAGCCCAGTAAGGCTGTGAGCCTCTCAGTCACTGTTCCCGTGTCTCATCCTGTCCTCA
ACCTCAGCTCTCCTGAGGACCTGATTTTTGAGGGAGCCAAGGTGACACTTCACTGTGAAGCCCAGAGAGGTTCACTCCCCATCCTGT
ACCAGTTTCATCATGAGGATGCTGCCCTGGAGCGTAGGTCGGCCAACTCTGCAGGAGGAGTGGCCATCAGCTTCTCTCTGACTGCAG
AGCATTCAGGGAACTACTACTGCACAGCTGACAATGGCTTTGGCCCCCAGCGCAGTAAGGCGGTGAGCCTCTCCGTCACTGTCCCTG
TGTCTCATCCTGTCCTCACCCTCAGCTCTGCTGAGGCCCTGACTTTTGAAGGAGCCACTGTGACACTTCACTGTGAAGTCCAGAGAG
GTTCCCCACAAATCCTATACCAGTTTTATCATGAGGACATGCCCCTGTGGAGCAGCTCAACACCCTCTGTGGGAAGAGTGTCCTTCA
GCTTCTCTCTGACTGAAGGACATTCAGGGAATTACTACTGCACAGCTGACAATGGCTTTGGTCCCCAGCGCAGTGAAGTGGTGAGCC
TTTTTGTCACTGGTAAGTGCTGGGTTCTTGCCAGTCACCCACCCCTGGCTGAGTTCTCTCTCACCCATTCCTTTAAAAATCTGTTTG
CACTGTCCAGTTTCCTCCCC
HUMAN PROTEIN SEQUENCE:hP27-007.2 (Seq ID No: 20)
MLLWVILLVLAPVSGQFARTPRPIIFLQPPWTTVFQGERVTLTCKGFRFYSPQKTKWYHRYLGKEILRETPDNILEVQESGEYRCQA
QGSPLSSPVHLDFSSASLILQAPLSVFEGDSVVLRCRAKAEVTLNNTIYKNDNVLAFLNKRTDFHIPHACLKDNGAYRCTGYKESCC
PVSSNTVKIQVQEPFTRPVLRASSFQPISGNPVTLTCETQLSLERSDVPLRFRFFRDDQTLGLGWSLSPNFQITAMWSKDSGFYWCK
AATMPHSVISDSPRSWIQVQIPASHPVLTLSPEKALNFEGTKVTLHCETQEDSLRTLYRFYHEGVPLRHKSVRCERGASISFSLTTE
NSGNYYCTADNGLGAKPSKAVSLSVTVPVSHPVLNLSSPEDLIFEGAKVTLHCEAQRGSLPILYQFHHEDAALERRSANSAGGVAIS
FSLTAEHSGNYYCTADNGFGPQRSKAVSLSVTVPVSHPVLTLSSAEALTFEGATVTLHCEVQRGSPQILYQFYHEDMPLWSSSTPSV
GRVSFSFSLTEGHSGNYCTADNGFGPQRSEVVSLFVTGKCWVLASHPPLAEFSLTHSFKNLFALSSFLP
HUMAN mRNA SEQUENCE:hR27-007.3 (Seq ID No: 21)
AATTCACTAATGCATTCTGCTCTTTTTGAGAGCACAGCTTCTCAGATGTGCTCCTTGGAGCTGGTGTGCAGTGTCCTGACTGTAAGA
TCAAGTCCAAACCTGTTTTGGAATTGAGGAAACTTCTCTTTTGATCTCAGCCCTTGGTGGTCCAGGTCTTCATGCTGCTGTGGGTGA
TATTACTGGTCCTGGCTCCTGTCAGTGGACAGTTTGCAAGGACACCCAGGCCCATTATTTTCCTCCAGCCTCCATGGACCACAGTCT
TCCAAGGAGAGAGAGTGACCCTCACTTGCAAGGGATTTCGCTTCTACTCACCACAGAAAACAAAATGGTACCATCGGTACCTTGGGA
AAGAAATACTAAGAGAAACCCCAGACAATATCCTTGAGGTTCAGGAATCTGGAGAGTACAGATGCCAGGCCCAGGGCTCCCCTCTCA
GTAGCCCTGTGCACTTGGATTTTTCTTCAGCTTCGCTGATCCTGCAAGCTCCACTTTCTGTGTTTGAAGGAGACTCTGTGGTTCTGA
GGTGCCGGGCAAAGGCGGAAGTAACACTGAATAATACTATTTACAAGAATGATAATCTCCTGGCATTCCTTAATAAAAGAACTGACT
TCCATATTCCTCATGCATGTCTCAAGGACAATGGTGCATATCGCTGTACTGGATATAAGGAAAGTTGTTGCCCTGTTTCTTCCAATA
CAGTCAAAATCCAAGTCCAAGAGCCATTTACACGTCCAGTGCTGAGAGCCAGCTCCTTCCAGCCCATCAGCGGGAACCCAGTGACCC
TGACCTGTGAGACCCAGCTCTCTCTAGAGAGGTCAGATGTCCCGCTCCGGTTCCGCTTCTTCAGAGATGACCAGACCCTGGGATTAG
GCTGGAGTCTCTCCCCGAATTTCCAGATTACTGCCATGTGGAGTAAAGATTCAGGGTTCTACTGGTGTAAGGCAGCAACAATGCCTC
ACAGCGTCATATCTGACAGCCCGAGATCCTGGATACAGGTGCAGATCCCTGCATCTCATCCTGTCCTCACTCTCAGCCCTGAAAAGG
CTCTGAATTTTGAGGGAACCAAGGTGACACTTCACTGTGAAACCCAGGAAGATTCTCTGCGCACTTTGTACAGGTTTTATCATGAGG
GTGTCCCCCTGAGGCACAAGTCAGTCCGCTGTGAAAGGGGAGCATCCATCAGCTTCTCACTGACTACAGAGAATTCAGGGAACTACT
ACTGCACAGCTGACAATGGCCTTGGCGCCAAGCCCAGTAAGGCTGTGAGCCTCTCAGTCACTGTTCCCGTGTCTCATCCTGTCCTCA
ACCTCAGCTCTCCTGAGGACCTGATTTTTGAGGGAGCCAAGGTGACACTTCACTGTGAAGCCCAGAGAGGTTCACTCCCCATCCTGT
ACCAGTTTCATCATGAGGATGCTGCCCTGGAGCGTAGGTCGGCCAACTCTGCAGGAGGAGTGGCCATCAGCTTCTCTCTGACTGCAG
AGCATTCAGGGAACTACTACTGCACAGCTGACAATGGCTTTGGCCCCCAGCGCAGTAAGGCGGTGAGCCTCTCCGTCACTGTCCCTG
TGTCTCATCCTGTCCTCACCCTCAGCTCTGCTGAGGCCCTGACTTTTGAAGGAGCCACTGTGACACTTCACTGTGAAGTCCAGAGAG
GTTCCCCACAAATCCTATACCAGTTTTATCATGAGGACATGCCCCTGTGGAGCAGCTCAACACCCTCTGTGGGAAGAGTGTCCTTCA
GCTTCTCTCTGACTGAAGGACATTCAGGGAATTACTACTGCACAGCTGACAATGGCTTTGGTCCCCAGCGCAGTGAAGTGGTGAGCC
TTTTTGTCACTGTTCCAGTGTCTCGCCCCATCCTCACCCTCAGGGTTCCCAGGGCCCAGGCTGTGGTGGGGGACCTGCTGGAGCTTC
ACTGTGAGGCCCCGAGAGGCTCTCCCCCAATCCTGTACTGGTTTTATCATGAGGATGTCACCCTGGGGAGCAGCTCAGCCCCCTCTG
GAGGAGAAGCTTCTTTCAACCTCTCTCTGACTGCAGAACATTCTGGAAACTACTCATGTGAGGCCAACAATGGCCTAGTGGCCCAGC
ACAGTGACACAATATCACTCAGTGTTATAGTTCCAGTATCTCGTCCCATCCTCACCTTCAGGGCTCCCAGGGCCCAGGCTGTGGTGG
GGGACCTGCTGGAGCTTCACTGTGAGGCCCTGAGAGGCTCCTCCCCAATCCTGTACTGGTTTTATCATGAAGATGTCACCCTGGGTA
AGATCTCAGCCCCCTCTGGAGGAGGGGCCTCCTTCAACCTCTCTCTGACTACAGAACATTCTGGAATCTACTCCTGTGAGGCAGACA
ATGGTCTGGAGGCCCAGCGCAGTGAGATGGTGACACTGAAAGTTGCAGTTCCGGTGTCTCGCCCGGTCCTCACCCTCAGGGCTCCCG
GGACCCATGCTGCGGTGGGGGACCTGCTGGAGCTTCACTGTGAGGCCCTGAGAGGCTCTCCCCTGATCCTGTACCGGTTTTTTCATG
AGGATGTCACCCTAGGAAATAGGTCGTCCCCCTCTGGAGGAGCGTCCTTAAACCTCTCTCTGACTGCAGAGCACTCTGGAAACTACT
CCTGTGAGGCCGACAATGGCCTCGGGGCCCAGCGCAGTGAGACAGTGACACTTTATATCACAGGGCTGACCGCGAACAGAAGTGGCC
CTTTTGCCACAGGAGTCGCCGGGGGCCTGCTCAGCATAGCAGGCCTTGCTGCGGGGGCACTGCTGCTCTACTGCTGGCTCTCGAGAA
AAGCAGGGAGAAAGCCTGCCTCTGACCCCGCCAGGAGCCCTTCAGACTCGGACTCCCAAGAGCCCACCTATCACAATGTACCAGCCT
GGGAAGAGCTGCAACCAGTGTACACTAATGCAAATCCTAGAGGAGAAAATGTGGTTTACTCAGAAGTACGGATCATCCAAGAGAAAA
AGAAACATGCAGTGGCCTCTGACCCCAGGCATCTCAGGAACAAGGGTTCCCCTATCATCTACTCTGAAGTTAAGGTGGCCTCAACCC
CGGTTTCCGGATCCCTGTTCTTGGCTTCCTCAGCTCCTCACAGATGAGTCCACACGTCTCTCCAACTGCTGTTTCAGCCTCTGCACC
CCAAAGTTCCCCTTGGGGGAGAAGCAGCATTGAAGTGGGAAGATTTAGGCTGCCCCAGACCATATCTACTGGCCTTTGTTTCACATG
TCCTCATTCTCAGTCTGACCAGAATGCAGGGCCCTGCTGGACTGTCACCTGTTTCCCAGTTAAAGCCCTGACTGGCAGGTTTTTTAA
TCCAGTGGCAAGGTGCTCCCACTCCAGGGCCCAGCACATCTCCTGGATTCCTTAGTGGGCTTCAGCTGTGGTTGCTGTTCTGAGTAC
TGCTCTCATCACACCCCCACAGAGGGGGTCTTACCACACAAAGGGAGAGTGGGCCTTCAGGAGATGCCGGGCTGGCCTAACAGCTCA
GGTGCTCCTAAACTCCGACACAGAGTTCCTGCTTTGGGTGGATGCATTTCTCAATTGTCATCAGCCTGGTGGGGCTACTGCAGTGTG
CTGCCAAATGGGACAGCACACAGCCTGTGCACATGGGACATGTGATGGGTCTCCCCACGGGGGCTGCATTTCACACTCCTCCACCTG
TCTCAAACTCTAAGGTCGGCACTTGACACCAAGGTAACTTCTCTCCTGCTCATGTGTCAGTGTCTACCTGCCCAAGTAAGTGGCTTT
CATACACCAAGTCCCAAGTTCTTCCCATCCTAACAGAAGTAACCCAGCAAGTCAAGGCCAGGAGGACCAGGGGTGCAGACAGAACAC
ATACTGGAACACAGGAGGTGCTCAATTACTATTTGACTGACTGACTGAATGAATGAATGAATGAGGAAGAAAACTGTGGGTAATCAA
ACTGGCATAAAATCCAGTGCACTCCCTAGGAAATCCGGGAGGTATTCTGGCTTCCCTAAGAAACAATGGAAGAGAAGGAGCTTGGAT
GAGGAAACTGTTCAGCAAGAGGAAGGGCTTCTCACACTTTCATGTGCTTGTGGATCACCTGAGGATCCTGTGAAAATACAGATACTG
ATTCAGTGGGTCTGCGTAGAGCCTGAGACTGCCATTCTAACATGTTCCCAGGGGATGCTGATGCTGCTGGCCCTGGGACTGCACTGC
ATGCATGTGAAGCCCTATAGGTCTCAGCAGAGGCCCATGGAGAGGGAATGTGTGGCTCTGGCTGCCCAGGGCCCAACTCGGTTCACA
CGGATCGTGCTGCTCCCTGGCCAGCCTTTGGCCACAGCACCACCAGCTGCTGTTGCTGAGAGAGCTTCTTCTCTGTGACATGTTGGC
TTTCATCAGCCACCCTGGGAAGCGGAAAGTAGCTGCCACTATCTTTGTTTCCCCACCTCAGGCCTCACACTTTCCCATGAAAAGGGT
GAATGTATATAACCTGAGCCCTCTCCATTCAGAGTTGTTCTCCCATCTCTGAGCAATGGGATGTTCTGTTCCGCTTTTATGATATCC
ATCACATCTTATCTTGATCTTTGCTCCCAGTGGATTGTACAGTGATGACTTTTAAGCCCCACGGCCCTGAAATAAAATCCTTCCAAG
GGCATTGGAAGCTCACTCCACCTGAACCATGGCTTTTCATGCTTCCAAGTGTCAGGGCCTTGCCCAGATAGACAGGGCTGGCTCTGC
TGCCCCAACCTTTCAAGGAGGAAACCAGACACCTGAGACAGGAGCCTGTATGCAGCCCAGTGCAGCCTTGCAGAGGACAAGGCTGGA
GGCATTTGTCATCACTACAGATATGCAACTAAAATAGACGTGGAGCAAGAGAAATGCATTCCCACCGAGGCCGCTTTTTTAGGCCTA
GTTGAAAGTCAAGAAGGACAGCAGCAAGCATAGGCTCAGGATTAAAGAAAAAAATCTGCTCACAGTCTGTTCTGGAGGTCACATCAC
CAACAAAGCTCACGCCCTATGCAGTTCTGAGAAGGTGGAGGCACCAGGCTCAAAAGAGGAAATTTAGAATTTCTCATTGGGAGAGTA
AGGTACCCCCATCCCAGAATGATAACTGCACAGTGGCAGAACAAACTCCACCCTAATGTGGGTGGACCCCGTCCAGTCTGTTGAAGG
CCTGAATGTAACAAAAGGGCTTATTCTTCCTCAAGTAAGGGGGAACTCCTGCTTTGGGCTGGGACATAAGTTTTTCTGCTTTCAGAC
GCAAACTGAAAAATGGCTCTTCTTGGGTCTTGAGCTTGCTGGCATATGGACTGAAAGAAACTATGCTATTGGATCTCCTGGATCTCC
AGCTTGCTGACTGCAGATCTTGAGATATGTCAGCCTCTACAGTCACAAGAGCTAATTCATTCTAATAAACCAATCTTTCTGTA
HUMAN PROTEIN SEQUENCE:hP27-007.3 (Seq ID No: 22)
                              MLLWVILLVLAPVSGQFARTPRPIIFLQPPWTTVFQGERVTLTCKGFRFYSPQKTKWYMRYLGKEILRETPD
NILEVQESGEYRCQAQGSPLSSPVHLDFSSASLILQAPLSVFEGDSVVLRCRAKAEVTLNNTIYKNDNVLAFLNKRTDFHIPHACLK
DNGAYRCTGYKESCCPVSSNTVKIQVQEPFTRPVLRASSFQPISGNPVTLTCETQLSLERSDVPLRFRFFRDDQTLGLGWSLSPNFQ
ITAMWSKDSGFYWCKAATMPHSVISDSPRSWIQVQIPASHPVLTLSPEKALNFEGTKVTLHCETQEDSLRTLYRFYHEGVPLRHKSV
RCERGASISFSLTTENSGNYYCTADNGLGAKPSKAVSLSVTVPVSHPVLNLSSPEDLIFEGAKVTLHCEAQRGSLPILYQFHHEDAA
LERRSANSAGGVAISFSLTAEHSGNYYCTADNGFGPQRSKAVSLSVTVPVSHPVLTLSSAEALTFEGATVTLHCEVQRGSPQILYQF
YHEDMPLWSSSTPSVGRVSFSFSLTEGHSGNYYCTADNGFGPQRSEVVSLFVTVPVSRPILTLRVPRAQAVVGDLLELHCEAPRGSP
PILYWFYHEDVTLGSSSAPSGGEASFNLSLTAEHSGNYSCEANNGLVAQHSDTISLSVIVPVSRPILTFRAPRAQAVVGDLLELHCE
ALRGSSPILYWFYHEDVTLGKISAPSGGGASFNLSLTTENSGIYSCEADNGLEAQRSEMVTLKVAVPVSRPVLTLRAPGTHAAVGDL
LELHCEALRGSPLILYRFFHEDVTLGNRSSPSGGASLNLSLTAEHSGNYSCEADNGLGAQRSETVTLYITGLTANRSGPFATGVAGG
LLSIAGLAAGALLLYCWLSRKAGRKPASDPARSPSDSDSQEPTYHNVPAWEELQPVYTNANPRGENVVYSEVRIIQEKKKHAVASDP
RHLRNKGSPIIYSEVKVASTPVSGSLFLASSAPHR*
```

TABLE 3

```
MOUSE GENOMIC SEQUENCE:mD27-009 (Seq ID No: 23)
GCTTTGGGGTGGAGGGTCGTGCTGCAGGGAACTCAGCCAGGCCCCAAGATGGACACTTCTGGGCACTTCCATGACTCGGGGGTGGGG
GACCTGGATGAAGACCCCAAGTGTCCCTGTCCATCTTCTGGGGACGAGCAACAGCAGCAGCAGCAACCGCCACCACCGCCAGCGCCA
CCAGCAGTCCCCCAGCAGCCTCCGGGACCCTTGCTGCAGCCTCAGCCTCCGCAGCCTCAGCAGCAACAGTCGCAGCAGCAGCAGCAG
CAGCAGTCGCAGCAGCAGCAGCAGCAGGCTCCACTGCACCCCCTGCCTCAGCTTGCCCAACTCCAGAGCCAGCTTGTCCATCCTGGT
CTGTTGCACTCTTCTCCCACGGCGTTCAGGGCCCCCACTTCAGCCAACTCTACCGCCATCCTCCACCCTTCCTCCAGGCAAGGCAGC
CAGCTAAATCTCAATGACCACTTGCTTGGCCACTCTCCAAGTTCCACAGCCACAAGTGGGCCCGGTGGAGGCAGCCGGCACCGGCAG
GCCAGCCCCCTGGTGCACCGGCGGGACAGCAATCCCTTCACGGAGATAGCTATGAGCTCCTGCAAATACAGCGGTGGGGTCATGAAG
CCCCTCAGCCGCCTCAGCGCCTCTCGGAGAAACCTTATCGAGGCTGAGCCTGAGGGCCAACCCCTCCAGCTCTTCAGTCCCAGCAAC
CCCCCAGAGATTATCATCTCCTCCAGGGAGGATAACCATGCCCACCAGACTCTGCTCCATCACCCCAACGCTACCCACAACCACCAG
CATGCCGGCACCACGGCCGGCAGCACCACCTTCCCCAAAGCCAACAAGCGGAAAAACCAAAACATTGGCTATAAGCTGGGACACAGG
AGGGCCCTGTTTGAAAAGAGAAAGCGACTGAGTGACTATGCTCTGATTTTTGGGATGTTTGGAATTGTTGTTATGGTGATAGAGACC
GAACTGTCTTGGGGTTTGTACTCAAAGGTAGGGACTGTGGTTTCTCTTTATACATTCAACAAAAGGAGTATGTAGACGAGAGAGAGA
GAGAGAGAGAGAGAGAGAGAGAGAGAGAGAGAGATTGATTTTGAGAGATTTCCTAACTTCCCATGTTTTTCTTCCATGGTCCTGTGA
GAATTCCTTTCTTGCTTCCCTTTGTGGGGACATCCCCACACACTGTGTCTAATGTGCAGACTCTGTGTTTGGGAGGATTAAAGAATC
CCTTCTGAGAAATCGCTCTTTTCCTCCGGGCTCACGCGTACTAAAGCATAACCCAGCAGCTTAACGCACCAATTAATTACACTCTGC
CTTGATGTGTTTGCCAGTTCCTGCTACTTCCCTCCTCGCCTCCCCTCCCTGCTCCACTCCATTCCCCCCCCCCCATAAAAAAGAAAT
AAATGTCTGGGTGGGGGTGGGGATCCTGTACCGTTCCCCAGCTCCCCCCCCCCCCCTTCCCAAGTTTCTATTTCCCTTCTCTTCCCA
GAGAGACTCCAGGGTAAAATGGAGATGTTCCCGAGTCTCTCCCAAGGGCACCGGGTTAAAAATATAGGTGGAGGAAATCAGCAGTCT
CTTCCCTCAGGAAAGAGGACTCAGAATAAACCTGCCGCTGCCTTGTAAACATGCCCTGATAAAAATGGCTACACGTGTTACCCAGGC
AGAGCATGGGCACCGCCTTGCCATCACTCGGGGAACGGGGAGCGCCTCTCCTTAGGATGGGATGACAGGGCTTCCCCAGCGGGTCAC
CGCGGAGTCGGCACCCCAGCTTAGCCCTGGTTCTTCCCTCCCACCCTAACTCCTTCTCCTTGGGATAAATAAAGATGAGTGTACGTG
TGTGTGCACGCTGGATGGAGAACGGCAGGCACAAGGCACTGTCTTGGGGGTGGGGTGGGGTGGGGGACGCTCCATTGCTCTATCCTT
TCAGTTGGAGGAGAGAGCTTCCTTCCCAGAGGGGAAGGAGGTCGAGGAAGGTGGGTTTTTGTCAATGCTGCATTTGCCAGGAAGCTG
CATGCCAGAAGGCTTGGAGGTGCAGGGAATTGTTGGGCAAACGGTTTCCCCTACTGTACCACTTGGTCCCAGTTTGCTGGTGTTTGC
ATGTGTATATGTGTACTCGTGTGAGTGCCTGCTTGTTCTGTGTATTTGTGTATTTGTTTGTCATTTTGGACAAGGGGTGGCTGGGCT
GGCTGGCGGGTAGCTGGGAGGGGCTCAGCTGTTGGAGGTTTCAAATAGAGCCTGTTTCAGGCAGCGCACAGCTGCTGGAGACTGTCA
CATTCCTTGTAGAGATCTAGTCACAAAAACCTGGGAGGTGCTGATCAGATTTCCCCCATTCCCAGAGTCTCCCTTTAACCTGATGTG
GGCAGTGCTCCTTGTCAGGCAACCTGCATGGACAGAGCCCAGACTTCCCAGCAGAGAGTCCTCCAGGGACCTGCTGCTTTGCAGGGA
CTGCCCAAGCCAGAGCACTTGGAAGCTGCCTCAGTCTAAATAGCTCCTGGATGAACAGACCTCTCCATGAGCAGAGTGGGGTTTCCC
CTGGAAGCCCAGCTCACTTAGTCATTCCCTGTAATGAGAGCCTGGGAGGCTGCAGGTCCCTCTGTTCCCTGCCTTCTTAGAGCTGCC
TCTCTTGCTGTGTGCTCTGCTTAGGAGATGGGCTGGCAACCCCCCCCTGGGAATTAGGAACACTTGTTAGGGGGAATCCGTGGAAGC
CCACATCCCGGAGGTGTGGCATGGTGCTAGCATGTTCTTTTCAGTGGCCCCCATGGCGGCTGTTAAGGAAATGGCTTTTCTAGGAGT
CAGTGGCTGTGTCCGGCTCTGAGTGTGGGATCCATGATCATTTGTCTGAATGTGTTTTGGGGGTAGGGTGTCACCGGGCAGGGACGA
CTATCTTCTCTGATGGAGAGAGGTTTTCCCGGGCCGGGAGGCTGTAAGTGTTGACTGGGTGATATCACCCAAGGTGTGTGCTTCCTC
CACTCCTAAGTCGTTGATTCTCTCGTGGCGATGGTACTGAGGGACAGTGACCAGGAAGCTCCTGCGTGCCTCAGTCAGTGAGGTGAC
CACTTCCTGTGTACCTCGGGGATGGGATTGAAACAACAAAATGTTTATTTAGTGAGGCAAATGTCCCTATCTACCTCGGAGGCAAAG
AGGGTCATATCAAAGAATAGGACCAAATCAGGAGTTACAGCTCTGTCACTGCTGGCTGTGTGATTCTTGGAAGTCAGGATACCCTGC
AAGGCTGCTTCCAGCTTTCTGGTGGGGAGACTGAGTCAGATGAGGTCAAGGCCCACCAGAGCACCTGCTCATGTAAGAGGGGTCGGC
TGTGGCTGTCTGTGATGACTGAGGAGAGAGACACCTTCTTTCTCTGTGATTAGATGGAAAGTAAGACGCAAGGACAGAGGAGAAAGT
TCTACAAGGAATCTGGGTCTGGTTGGAGACTGTCTTTACACATGAGGCAGCTGTTGGGGAGAGAGGTGTCAGCTCCCCAGCTTTGGG
ATACAGGGGGATGGGCTCTGTGACAGAAGAGCCCAGAGACACTCAGGGTGGATGAGGGACTTTCCTTTGCTAGGATTCTTGTGTGCC
TGCCTGTCTTTGCTGATGATTCTGGAAGCCCGAGGATAGACCAGGTGACCAGTCATTTTGGGTACCTACCATGGCCATTTCTATTTA
AAGGCCCTGGTGAGGGACAGGTTCTTTTTCCTCTGAGTGACTGATGGCCTGCAGCCCTGGGCCTTACCTAACTCTGAACTTCTCTGC
TTTATCTCGTGAGGCCGTACCTGCCCCGTCCACCTACTGTACCTGCTGCTGGGAGAGTCATTCATCGCATGATGAATGCAGGTCCAG
GGGTCTGGGAGAAAAGCAATGGGGCAGATACCCTGAGTGTGCTCAAAGGTGATGCAAAAGGGAGCCAGCGTGAGGAGGTTGAGAAAT
GGGGACAAAGTGCTTCCTGGCTCCCACCCTCACAGGCTGTAGGCGATGACACTGGCGGGAAGGAAGTTCCACTGAGAAAGGAGATGG
AAGCCTGACTTGGGGAGGGGGGCGTTGTGGACCCCTAAAGGTGCAAGGCTGTCTCTGAGCTGAATGGAGGCTCTGGTTTATCCATCC
CTCCCTCCCTTCCTTCTCTTGCTCCTCCCGTTCTTCCCTCCCCTTCTCATCCTCCCTCACCTTCCTTCCTTTAGTAGGTGGGCTTCC
TCTTTGGAAACTTAGACAACAGGAGAGGGCTGATTCTCAAGATCCTGTTTGGAAGTCTTGATGATCCCGCCTCTGCCTATGGCTCCC
TCTAACTGTTAGAGTCTAACAGGGTTAGGGGATGGGGATTTAGGTTGTTTTTTTTTTTTTTTTTCTGACTGTGTTCTTTAGTTTTTT
CTGGAGCTGGTTTGGCCCATGTCACCCTTTTACCTTAAGCTGTCAGTAATTCTCTACTAGATCCAAAGAGTTAGGGGTAACCTGGCC
TGCTTTCTCTTCCTTAACACCCTGACAGGACGTCAGGCCTCAGCCTGTGGCAGCTTCCTCAGTCTGGGCAGCAGGGTAGCTGTCTGT
TTCCACAGATTTTGTACAGCCTCCGTAGCTCTCCACTGCTGGCCTAGAGATCCATTTCAGCCTTACCTCTGCTCCCTTGCCCCTCCT
GCCTGCCCAATCAGACTTCCTGTCCTGTTCCTTTCTGATACCCCAGCTTGACCTGTTGCTAGGATGAAGCAGTCACCGATACAGAAG
TCCCTATCTGCTTCCTTGGGTACATTCTCCTCACTTCCTGTTACTGTTCCTTCTTCAGACCACCAACTGGGCCGCCACCCTGAGCTA
GGTCTACTGAGCAGCTAAAGGAGGTCCCTACTGCATACTCTCCAGAGAATGGTGGCTTCTAGGCCTGGGCCAATTTGTTGCCTATCT
CTTTGCTGGCCTCACATATATCATAAAGTCCCCCCAGCCTTCAGCTGGAATGATGGATTGTATTCTAGGCTTTTATTTTTGGTCTTT
GTAGGGTGTGTGTGGGGAGGTGGTAGCCACAGCTTCACACCGTAGGTAAACATTCTACCGAGCTTGTCTCCAACATCCAAGATGCAT
CTCTGTCCCGATCCTCTGCCTTCCTGGGATGGAGGCCCTAGTTGCACAAGGACAGAGTGGTAGCCAGGGTACATAGGATGAAGGACC
CTGCTTGTCTCTTTGGGGCCAAGTGCCTTCTGAGGTTAGGTTACTGGTTGCTGGAACATGTATTTCTGGAGGGTTTTGGGGGGCACC
AGTAACTATGATTTCAGGCATGCTGGGGAGGTAGACATAGGTCAGACCCTGGTGTGCACCTCCTGAAGGATGTCCTCTGAGAATAAG
AAGGCAGAAGACTCAGTAAAGCACTTTGGCTGCAGGACCCTGAGAGCCAGCAGCCAAGGGCCCCAGGCCTGCATTTTAATTGTCACA
GGGTATACTGGCCATGCCTGCCCTGGGGAAGACAAGCAGATCTGATAGTATGAGACAGCCAACCCTGCACCCATGGTGACCCTTTAT
TTATATTCCTTGTGGACAGTGAACTAATGAGTCAGATCTTGGTAACCTATAGGATTACCAGTAACTTGTCACCTACTTTCCTTCCCA
TGTCACCCACTGTTTATAACTGTCCTGGGGACAGGCTCCACAGGGCTTCCAGACTTCCCATCCCCACAGGCAGCACTGGGCATTGGG
ACAATGGCTGGGCATTTGTCTTCTGAATAAGCTAATTAGAAAGCTACTCCATGGCCTTTGATTCCCTTATTTAGCTTCCAGGAAGGA
GGATGTATGAGGCCATCCTCAAAAGATTCCCTTAAAAAAATTAGGTAGAGAAAAGCAGAAGAAAATTGGAGTAACCTGTGTTGTGAC
TCAGGCTCCAAGGCAAATGTAAAATCAGAACAGAGAGCTCTGGTTGCATGCCAGTTACTTATGAAGCACACTGGGGGGAAGTGTGTG
TGTGTGTTGTGAGGGGCGGCTGTACTGTACTGTACTGTACACAATATCCCCTTTCTTTGCGCCATGACAGGACCACCAGAGCATTCG
CTGGGTAGTTGTTTTTCTCTCAAAACCCCAAAGGGAGAGAGAGAGAGAGAGAGAGAGAGGGAGAGAGGGAGAGAGAGAGAGAGAGAG
AGGGAGAGGGAGAGAGAGAGAGAAGAAAGCCATCAGTTCCTCTTTAAATAATGAGGGTTGGTAATTCTAAGACTGCTCACTCTCAGG
AGCCGTGGGGAGGAAAACTCGAGTGCCTTTTCTTGTGTGGAAAGTCCGGAGGAGGCTAATGGGTTTTCAAGGCCCCCTCCCCTTGTC
ATTCCACCCCCAGGTGCTCGGGAGGCAGGGGAGAGGCTGGGGGCAGGGACCACACTGTTTCTTTCTGCCCCACACTGCTCCTGCGTG
AATTGCTGCTGAGTGACAGCTGAGGGAAGGCGAGGACCTTTTTCTTAAGAACTGGATGACTTTACACCCAAGAGTCCCAGAAGTAGG
TGGTGCTGGAGGCATCCTATGCAGATGAAATCACTCACTAAGCTAACACTGCCAGTGGGCGTGGTACTGAGATAAGAATCCTGCCCT
TTTGACTCTAAGTTTCTGTGCCTGCTGCTGGAGGCCTTGCTTACATTCCTTGTGTGTGTGTGTGTGTGTGTGTGTGTGTGTGTGTGT
GTGTGTGTGTATTTGTGTGTGCCCGCATGCGCGTGCGCGCGCGTGCATATATGTTTCCAGTTTGGAATTTCAGTGCAGAACCTTGCA
CCTATTAGGTAAACAGTCTGCAGAGGAATCTCCCCAGCCCAGATTGCTTATACTCTGTTTTGGTTTTGTCCTTTTAGATTGCTTGCA
TTTTCTCTGCATGTGCTGTGTTATATGTACATGTGCATATAACATGTGTGCATGTGGCTGGCCTGCGAGCTCAAGGGCTCTCGTCTG
CTCTCACCGTATCCCATCTACCCCTGTATCCTAGTTTTCATATGGATACTAGAGATTGAACTCAGACCCTTGTGCTTGCATGGAAGG
TGTTTTGCTGACGGAACCATCCTCCTGGTTCAGCACCCCTCCAGCCTCTGATATATTCTTGGAAGAAACAGATCAAAATCTATGGGC
CAGAGACCAGAGATACAAAATGTCGGGCTAGAAGAAGCTTGGCCAAGACATAGGCAGAACACGATTGGGATGGGAGCTGTTGGTGCA
GGGACCCAGACCAAAGGTTCTGTGGTGCAAGCAGCTTCCTGGGGGCAGGCAGTCTGTTCATTGAGGTGGACGGAAGGCCCATGGTGG
GTCCCTGAGATGTTTCCTCTATGGGGGGGGGATGTGGCCATCCACTGTCAGGGTGACTCTGAATAAATTGTGAAGAAGTAGTTGTGG
GGATTACAGGGAAAGTGAGCTTTGTATAGGTTTGCTGTAGGTACCTACCACAGCACCATTGCCCCGCCCCCAAGCCACTGTGCCTCC
CCCTCCCCTCCTGAACTAGCTGGCTTACCTTGTTCCCTCCTGCCTGGATCTTCTTTTAATTTTCTCCTTCCTTTCTAGTGCTAGCTG
CCGAGGTGGTAATGGGGAGTTGGGGATCAGCATGGTGTATCTCQCAGGCATAGTCCAGCTGCCGTTTCGCATGGTCCCCACAGTGGT
TCAGGATCGTTACAACCAACAGTACTTTCCAGCTGAGAAAATCGAGCCATCAGACCCCCTTCTTAGATGGTTCTAGAAGGAAGCTCA
GAGCTGAGGTGCACGACTAGCTTCTATTTTAATTTTCATTTCATTTATTTATTTCCAAACCAGAAAGCTGGGAACTGAGTAAGGAAG
TCTCTGAGGGCTTTTCCATTGCTATTCTAGAATTTTCTTCCTTCTGGAGTTCCTCCTCCCCCCCTCCCCCCTCCCCCTCTCTCCCGG
CTCTCTGTGCCATCAGCATAGCTGGTGCTGGAAAGGGTTCGTCTTTGCCTTTATGGCACCCTAAGGAGGTGCTGGTACTCGGTGCTT
CTTGCGATTGTTCTTAGCTCACTTAAACCTGAGACCTGAGCTCTTTTTCTGCCTCCAGGTCTTGTCCTTGTTCCAAGGACTGTCTGA
GCCACTGTTCTCAAAGCCATGCTCATCAGAGGTGTCCCTCAGTCAGTCAGAAGGCCAGACAACGTCATCCAGTTTGAGAGGAACTTG
GGCTAGCCCTGCTGGAGGCTCCCTGTAGAAGGTCGGGTAGTTACTGAAGGGTCAGATGAGCCATTGTGGAAAGAGAGACTCATTTCT
GTAGGACAGGGGGACAGAGGATAAAGTGTGTGGGAAATGCAATTTATGCAGATATCTTGAAGATGCCCCAGGATGTGCATGCAAGGG
CAGATGGGAGGAAAGAGAAGATTGTGAGAGTTATGGGGGCTGTGCTTCTCCTGAGAGGTTCTGAACAGGGATCAAAACACTCCTGCC
AGGGGTGTGGGTTCAGTGATTGACTTAATAGTGAGTTGCCAGGAACTCTAAAGATCCAATGCTCTTTTCCCCCTCAAATATGATCTT
ACCTGTTACGGATGGCCTAGTATGGTAGCTAAGGGGTTTGCATCTAAGTTTATTTCTTAACTCTATGGCATACCTGTGGTGGGGAAT
TTGCTGAACCCTGTTTCTTCATATGGGAAGAGCGAACTCCAAGCCCTCCTTAGATCATAGGACTCAACACTTTTCCCTGCAATATAA
CATCTAGAATCCTTCCCTGTACCATAGCATGCAGCATCCCTCTTTGAAGCACAGTCCGTAGCACTCAATACTTCTGTCCGTATTGTA
GTACCTAGCCTAGTGAGTACCCTGCAGTGCAGCACTGAGCTGGGCGTGCATTGAACTTCAGCAGCTGCCAGATGTTGCTTTTATTGG
ATGCCGCTTGGAGGACTGAGCCTGGAGGGACACAAGTACACATGTCCCCTGTGTATGCTTTCCAGGTGGTTGTGGAGAAGAGAAGAT
GGGCTTCAGGTTAGGAGAAAGGACCCTGGAAGAGTTGAGCACACGGGACCTTGCCCTCATTCTCAAGAAAAATGGACAAGCCACCCA
GTGTTACTGCTGATAGGTCTCAGTGTGTAATGAGAGGAAGGGCTACTCTTGGGAAGGCGGGACTCTCATGATGGCTTGGCCTGGCCT
TCTAGAGTCTCCTATCATGAGTATATTCAGGGTTGGCAGTCTGTGGCTTGGGCATAGCTAATTGGGTGTAGGCATTCAAATCGGACA
GGTGGAGAGAGCACAAAAATAGACCCATGGGGCCAGGCCAGAGGCTGAGAGGAAGGTCAGGGGGACACATGATGATCAAGCAGAATG
AGATTGAGAGCTTGGGACTCTAGGGCACGAACTAGCCCACAGACAGATCCAGGCAAGTGAAGACTGCTACATCTTGCAGAAAGTTCC
ATCAGGAAAACAGTTTCCCTTCAAGACCAGAGACAGGGATACTTTGTATAACTTAAAAGGTTCCAGCCTGGCATGGTGGCACAAGCC
TTTAATCCCAGCACTCGGGAGGAAGAGGCAGGCAGATTTCTGAGTTCAAGGCCAGCCTGGTCTACAGAGTGAGTTCCAGGACACCCA
GCTCTACACAGAGAAACCCTATCTCAGAAATTTAAAAAAAAATGGTTCCTCTACCTCTTTTACCCTGCTTAACACTAAGGCCATGTT
CCTAGCCAGGCACCAGGGGGCACCAGCAGTAATTGGGTGGCGGAAGCTATGCCAGGGAACCTGGACTTGCTGGGGAGGAAGCAATTC
CTCCCATCCCAAGTCCCTATCCCAAACTACTTGAATTATATTCTGAACAGAGCCTTGGTGATTCAGGCAGAAGATGTGTGAAACAAT
ATGCAAATTGGCATCTGTTGTTGTGGCGTCAGGTCGATGGGCATTTTTCTTAAAGGACAAGAAGGTCGTTCAGATGTCAACTGAGCA
TGAACATTGGCTTAGACGAAGCTTGGGGCAAGCTTGTTGCTGAATGGGCTGGCGCTTGGACGTTGGAGCTATAGGTTCTTCCTCTGC
CACCTAGCATGTTCAAGGCTAGTTATTCGGGGTTTAGCCTGGAGAAGTCTGTAACAATGAACACCTTGCACAGAGGGTCACCCAGCA
AAGAGGGAGGAGGCTGGAAGATCATCTGATCTCCAGAGCCCTTTCCTAGTGGGGAGCTTGGGACCGGAGAAGGCTGCAGGGAGTCAG
GACTGCTCTGGGTTCTAACTATGGCCTACCCTTACTGCTGAGTCACCCTAAGGACCATGACTCAACCTCTTTGAGCTTCAGTTTCCC
CATTTGTAAATGGGAATACTGACATTCACTGGCCCAGTGAGACTCTCATTTGGTGATAGTTCAGTAAGTGGCCAGCGCAGTGGCTGG
CTGACACCTTTCCCAGGCTGGCGGTTCCTCCTCACACCTTCGCCTGAGCTGCCACCTGCCCACATTCCAGCGAGGTTTTACTAGCAG
GGCTTTTTTTTTCCCCCCCTGGGCCTTCTCTCCTTTGTGAGGTTTCTGATGTTAACAGATTTTTTTGTTTTTTGTTTTTTAATCTAT
TCTGACTTCCAGCGGGTGAGATGCTAACTTTTCACACAGATCTTGCACATGGAGACATCTGGAGCTGCTGACAGAGGTGCTGTGCCT
TCTCATGTTGAGAGTGTGAGAGTGATGCCATGGTTTAGGCCACCGTGTCGGGAGGGGCTCGGACCTGGAGGCCATTGGGAAGAGGAC
CCTAACAAGCCTTCTCTGGGGATAGCTGGGGACATTCCTGCCTTAGGTAGCATCCACCAACCTGGGTGGGCATGACTCCTGCACAGG
GCCCACTTCCATGAGGAAGGCAGGGCATGGAACACCGGTCAGGACCGCAAGGTTCAGAGAGGTTATGTGAGTTATAGGACCGCAAGG
TCCAGAGAGGTTATGTGAGTTATCCAAGAGCACTAGAGCATTGTAAGAAGCAGGGTACAGCAGGAACCCAGGGATTCTGGGTTTTGT
CCTTTCTTTCTTTCCCGTTCTCCTACCCTTCTCCAGGCAATCTAGGTGCTAGGAGGGGAAAATCTGTGACCTAGAGGATTAGAGCCA
GCTCTGTCTGGGGTCTCTCCTTACCACTACTCTCTGCCATCTCATCTATCTATGTATCTTTCTCTGTGTCTATCTCTGTATCTTCCT
ATGTAGGTATGTATCTCTGTATCTATCTACGTATCTACCTACATATCTACCTATGTATGTATGTATGTATGTATGTATGTATGTATG
TATGTATCTATCTATTATCTATCTATCTATCTATCTATCTATCTATCTATCTATCTTCTATGCCTCTTTATCTACCTAACATGTATG
TATGTATCTATGCATGCATGTATGCATGTATGTATGCGTCTATGCTGGGAAATCTATCTATCTATCTATCTATCTATCTATCTATCT
ATCTATCTATCTATCTATCTATCTATCTATCTATCTATCTATTGTGAGAATTGAACTCAGGACCTCTGGAAGAGCAGTCAGTGCCCT
TAACTGTTAAGCTATCTCTCCAGCCTGCCATCTCTTCTTAAAAGTGCTTATGTGGGAGACAGAGGCAGCAGATTTCTGAGTTTGAGG
CCAGCCTGGTCTACAGAGTGAGTTCCAGGACAGCTAGGACTACACAGAGAGATCTTGTCTTAAAAAACAACAAAACAAAACAAAACA
AAACAAAAAACAAAGCAAAGAGTGCTTATGCTTTCCCAGCTTGGCAGCAGTAACTGATTGCTGAGCTGGCTGGCTTTGCAGAGCAGA
AGATAGGATTTTTAAATGCTTATTTTTTAATGATTTATTTTTATTTTATATGCACTGGTGTTTTGCCTTCATGTGTCTCTGTGTGAG
GTCTTCAGATCCACTGGAACTGGAGTTACAGACAGTTGGGAGCTGCCATGTGAGTGCTGGGAATTGAACCCCGGTTCTCTGGAAGAG
CAGCCAGTGCTTCCAACCATTGATTGAGCCATCTCTCCAGTCACAGAAGGTGGGATTTTTAATTCCTGTTGTTGAGGGTAGCGGTGG
ACTAGAAGTGGATGAGGCTGGGGATGAAAATCACTGGTCTGTTGTTTACCTGACTTATTCAAGGCACTGGGTCTCATGTCCAGGATG
GCAAGGAGAATGTGAGCAGGCAAGCTGGACAGGCATGGCCTCTTAGGGCTCTGGTTCTTGGCAGTGATGACTTTTGCCTTTGATGTC
TGTCCTGAGAGTCTGTGGCCTTTGTTGGCCCGTGGTTCCCACTCCTCCTTGGTCCAGACTGCTATCAAGAATCCTGAGATCCCTCGG
GAGTGAATGGGTGGGAGCAGGGAGTCAGACTCGGATGTAGGCTGAGTCGGGAGGGTTTCATCATGGGCAGGTGACATCATAGACAAT
ATTTTTGTCCCCGCAGCTTGGGAGGGTACCCCACAGTCTTGAAGCAGATGCTTCCTCTCTCTTCCTTGGAAACAATAGGAGGCCAGT
AGGATAACGCAGGCTCAGAGGACAAAGCTGCTCTATGCCAAGGCTGACTATCCGAGTTCAAACCCCAGGTCGGGGAAGGAGAGAACT
AGTTTCTGTAAGTTGTCCTCTGACCTCTACATACTCTTCCCTAAATGAGAGAGAGAGAGAGAGAGAGAGAGAGAGCGAGAGACTGCA
AGTGGGTATGGGTATCTAACTCTGATGAAGAGAGGAGACAGAAAGGAGAGAAGACAATGAATATTTCCAAAGGGTCTATTCCGTGTC
TTCACATTCCTNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNTCCTTTCCTTTCCTTTCCCTTTCCTTTCCTTTCCTTTCCTTTCCTTTCCTTTCCTTTC
CTTTCCTTTCCTTTCCTTTCTCCTTCCTTCCTTCCTTCTTTCCTTCCTTCTTTCCTTCCTTCCTTCCTTCCTTCCTTCCTTCCTTCC
TTCCTTCCTTCCTTTCTTTCTTAGTTTGTTTGCTTTTTTTTTTTTTTTTTTGAGACAGAGTTTCTCTGTGTAGCTGTAGTTGTCCTG
GAACTTGCTCTGCAGACCAGGCTGGCTTTGAATTCTGAGATCCATCTGCCTCTGCCTCCCAACTGCTAGGATGAAAGGTGTGCAACA
CTACCCAAGAGTGTGTTCTTTCAAGCCACCCCCCCAAAAAAAACCCTATTCAGTCTGTCAGTCTCACCTGGCTCATTGTCCCCTTAT
TTTCCTTCTTATTGGCCTGCTCACCTGACCCGACACCCCTGGGGTGTGCGGAGGGGCAGGCAGGGTGCTGTCAGTGTGATGCATAGG
TTTGTGGGAGCTCTGGGAGCCAGGTACAGGGAAGGTTGGGCTTGGACATCCTTTGAGTTTCTCTGTCCCGGCTCTGTGACTCTGTGA
GTCTCAGTCTCTAGGTTTACAAATGAGGACAATGGTGTTTTCTCTCTAAGAGAGTGTGTGTTGTGATGGAGACAAGATGAAGAGAAG
GCTTTGGGCAGGGCATGGTGAGTGTCAAATAATATCACATCAATAATGTTCTCAAAATCGACTTCCAAACCTGGCTTTGTTGTCCCC
TCACCTGAGGGCTAGGATGGGTTCCGCATCTTTCCTCCTCCAGGCAGTCTTCTGGGCCCTGTGTGCCTACCTGCTATCCTGCCACAG
GTTAGGTGAACTCTCCAGATCTCAGCCCTGTTTATCACTTGCTAGCTTTAGGAAGTTGCTCTAACAGATCGACTCGCAATGTTCCCA
GTGGTTCCTGGTAATCTGTCTGTCAGTCTTCTGCAGGAAAACGCCAGGCTGCTGTCAGTAAGCTTGTGACATGGCTTTTGCCATACC
CTGGGCTCTCAGTAAGGGTCAGCTGAGATTGTGGCGGCTGGTGGGAACCTCTCTGGCTAGGCATGGCCAAGCTTGGCGGCTGGCCAT
TTGTTTGGTCATCTGGAGAATGGAGCAGGGAGCTGGAGGAGTCTCCTTCCATCTTAGGCTGGTGACCACATGGAACTGGGGGAATGA
ATACTTGAAAATGGCAGTGCTCTGGTGACAGGGCACTGCCTGTCAGTGAGTGCAGGGTCTACTGGAAGAGTGGGTTGGAGGGTTATC
TTCATGTCCCATGTGTGAAGGTAATGCAGCTAAGTGGATTTCCTCACTATCAGGAGATGGCTGAGAGACAGGCCCTTTGGCCAGCTG
CCCTCCCTGCTGCCTCCTCCCCGCAGGCCAGCCCTTGGCATGAGGCCTGCTTGTCTCCTGAGCCCTCTGCGCAGCAGGGATTTAATT
ACTATGATGTAAGGAGCCCAGGCTCGAGGCTCTGGCAGAGGCGAAACAGATTTGTCTTCTTACTGGGAGAATAAAAACGGACAGAAC
AGAGAGAAATGACTGTCCCCAACCTCCCCCTCCCCCAGAGGTTCCAAGGCTGTTGAGCTGATGGGGAGATTTTTTAGGGGATAAGGG
GGGAGGAACAGGTAGCAGTGTGTGTGGGGGGGCGGGGCAGGTGCAGGATACCCTGTTCAATTCAGAGCCTTTGCTATTCAGTTTTGC
CTTCTATGACTAATTCAGCTAAGCTGCCTGTTTTCCCAAAGACTGGGCAACAGCCTCCATCAAGTACCTACTATGCGCTAGATGTTG
GCCTTGAGAAACTTCCTGTCCAGGCAGGAAAGCATGTGTTTTTATGCAGCCAACAGATGGCTCCTGACCACCTGTGAGGGGCCAGCC
AATTATTCTAGGCACAGCACAGTAGGAAAAACCACAGTAGGCCAGAGTCTTGCCCTTGGGGATGCACATTTCCATAGCTGAACCCAA
CAGTAAAAGATATCAGCTAATTATATATTGTGTTTGAAGTCCTATGAAGGAACAGAAAGCTGGGCTAGCAGGACACGGCGTTCCACA
GGGAAGCTAGTCATCTCGAATAAGTGCTCAGAGGAGGCCTTGATGAGAAGGTGACATTTGGTGAAGGTCTGGGGTTGGTGAGCAAGC
CAGGCTTCCTTGGCCATGGAGGTGGGGACTAGCTTCTCCAAGGCCTTCGTCAGGTTTGTGCTTGCTGTGGTCTGGATAGTTCTGTCC
AGTAGACCTAGAACACGGAACTATCTTGGTCCTTTTATGCTTCCTGGTAGCTGTATATTTTTAAAAGTAGGACATATGAAATTTGTT
AATATGTTCATTTCATGTGATGAAGAAAAATATTACAATTTCAATGCGTAAGCAACATAAATATTTTCAATGAGACATTTTCCATCC
TATCTGCCATGCTATGTCTGGTGGATATCTGATGGCTCATCTCAGCCAGCCACTAAATCTTCATTGGAAACACTTACCCGGTTTTAG
AGTCCATAAAATTTATAGTTAAATAAACACTCACGTTGTTGCAAGCATACTTTAGGAGTTTCTAATAAATTAATGGTGAACCAGTTT
AGAATTTATGGTTATTTATTTTGGTACTAGGGGTTGAACCATGGGCTTCACATGCGCTAGGCACATGCTAATACTGCTGAGCTATGT
CCCCCACATCCTGGTACATCAGTTTTCAGATGAATGATTAATAACAGAATGTACACATAAATTCCTCACCCTCGCCATGTCAGTGGG
GTGTGGTGGAACGTGTGGCATCCATCAGAGACTCCCCAGGAGTGAGGAGCCCCTCCCTCTGCCCGGCTGAGCATAGGGAGCACGGGG
ATGAAGCCAGCTTGGAGGTAGGACTTGGAGTGTTCTGTGCAGCACTTGCCCTTTACCCTGGCAAGATGGCATCAGGTGAGGGCTCTG
AGCTTTGGTATTAGAGTTAGGCACAGACAGGGAAGCCTCACCAGAGGTTTAGGGACACTTCAGGTGAGGCTGGTGGTGATGGAGCAC
CCAGGTGGGAAGTAGCAGCCAGGTGGTGAAAGTGGGTGGTCCTCGCAGTGTTTGGAAGGGTAGGTAGGCTTCTGGTGTGTCTGCTGA
AGTGGGGGGTGGGATGGGCAAGGGGTAACCCTAGAAGCTGGGAGATTTGTGAGACCCACACTTGGGTTATACAAGGTTCTTGCCCAG
TCCCTTTCTTTGGTGGACGGCTCCCCTTGCATTCCAGAAGACAGTTTGGGACAAGGCTGGCATCCTTTCAAACAAAACCAAGGGGTG
GGCAAGATAGCAGATCAGGGTCAGGGTTCTTATGGCTAAGCCTGTTAACCGGAGGCGCCCAATCCCTGGAACCCACATGGTGGAAGG
GGAGAAGGGACTCCCTCGAGTTTTCCTCTGACCTCTACATGTGTGCCTTGGCGTGGCTAAACCCCCAGCACCACAGAATAAATAAAT
ATTTAGAAAAAATTAAGAAGGGGCTGAGGAGAGAGTTGCACGGGGAAAGCTTCTGTAGTATGTGTGTGAGGACTCACCTTTGAATCC
CTCCCCCCAAAAAACATTGAACCCATATAAAAATGGATGCGGCAGGGTGTGTGTCTGCAATCCCAGGGCTTCTGAGCGAGGTGAGAA
TCAGAGACAGGAGACTCCCTGAATGCTCACAGGCCAACCAGCCTAGCGTGTGCAGTTGTGAACAGCAAAGAGATTCTGTTCAAAGGA
AAAAATGTGGCATGGAAGAACTACGCCCAAGGGCCATGTGCACACCATGCCCCCCGCCCCCAACCAAGAAACAAAGCGGCACACACA
GAACCCAAAGAAATTATCGGTGCTTACAGTGTGATTAGTGTTTAGTTCCCAAAGGTCACAGGAATCTAATAGTCAGTCCACATTTGA
AGATCAAGTCTCCCTGTTCTGGGCTGACCCCAGAATGCTTTGATGGATAGGGTCAGAAGTGGCCAGAGATTCCCATGGTATGACCAG
GAGATTACTGAGATAAGATGGATCCCATGGATTCCTAGTGTTCAAACACTTTCAACTCTGTGGGCAGTGTGTGTGTGTGTGTGTGTG
TGTGTGTGTGTGTGTGTGTGTGTGTGTGTGTGTGTAGATCAATGAGCTTGAAAAGATAGCTCAGAGGTGAGGTCTAGGTGTTGGGAG
GTGCCAGTCTTCTGCTCACTAGCCAAACTGCTATGTCCAAAGCCCGTTGACTCTCCAGGGCTCTCAAGGACCACAAAGCCATGCCAC
TATAACTGTTTCCTTTCTAAGTGTGGAAAGAGCAGGGATGGCTTTAGATGTCTTCTCCTGATGTCTTGATATCTGATGGTTGGCCTT
GGGCCCAGAAGGTGAGGGTCTGAGGCTATGGGACCTGTGGGCCAGCTCCTCTCACTGGTCAGTCAGATATGCTGGGTGTGGGTCTGC
TGCTGCATTCAGAGTTGGTCTGTGCCACTAGTTGACCATATGACTTTAGTACCTAAGTCTTCATCTGCGTTTGCTCATTTTGTGGTG
CAGGCAAGGGTGATGTAAATAGATTGTGGTGATCCGGCTCAGTCAATGTTGACTCCTTTCTTCTTCTGTGAGGTACCTCAGGAATAC
CTCACACAAGCTTCTCTGGCCTTTGGTGAAGAGAGACAGCCTGTCTGGGGTGGGATGTTGATGAGGAACAAGAGGCAGGGGAAGGTG
GTGATGGGGAGACCTGGGATCTACCTGGCCTGTGAGCCAAGGCTTTCCACCTGAGGTGCTTCTCAGAATACATGTAGCAGAGTCTGG
AGACTTGGTTGGTCATCACAGCCAAGGGAAGGATTTGATAGATACCTAATGGACAGGGGCCAGGAATACTCACCAAATCAAGGTATT
ACCTGGTTCCAAACAGCAGAGACTGGAGAAGCCTGGATAGATGGCTTGTTACAAGGACAAATGGAAGGATGAAACCGTCATCCTAAC
TATAGCATGACACTAGTCATCTCTGGGATGCTGGACTAGGTGGGACTCAAAACTTCCATTTTATTTTGAGGGTCTCACTATGTAGGC
CCCTGCTTGTCCAGAACTTGCTAGATAAACCAGGCTGGCCTCAAACCCACAGAGATCCACTTGCCTATGCCTCTCAATAGCTGGGAT
TCAATGCATGTACAACTATACTAGGCTCTCAGATTTGAATTCATGATCATGTTCAAATTCATGTTCAAGTTCATGCCCCTCCAGAGT
TTTTTCTTTGTTTCTAATCTCTGTGTCTTTACATCCTTCACCTTGGAGGTTTTATCTGTAGACGAGGCTGAGGTATGAGCCACATTA
GAGCCTGTGAGAAGGTGAGACTTAGGGGCTTTTCCAGGACTTCCTCTTACCTCCAAGAGGCCTCATATACTGGCCCCACTTTATTTT
GGTCTTTTTTAAAAAAATGTGCATTGGTGTTTTGCTTGCGTATGTGTCTGTGTAAGGGTGCTAAACCCCCGGGAACTGGAGTTACAG
AGAGTTGTGAGTTGCCATGTGGGTGCTGGGAATTGAACCCAGGCCATCTGGAAGAGCAGCCAGTGCTCTTAACCCCTGAGCCATCTG
TGCAGCCCCACATCTCCGTCTTTGTTGTTTCTGAGAATGAGTAGAGCTGAAGTCTCCATTTAAGAAAGAGGGGATTTTCAGGGCTTA
CGACTCTGTGAGTGGGAGACTCAGCTGGTTTAGTGGCAGACTTCCCGGCATGGGGAGAAACAGTTGGTTTGCCAGCTTGGGTCAAGC
TAGAAGCCATTTGAGGTTGTGTGGGTCACTGCTCTCTAGGTCTACCTGGGGAAGCAAGTACTTGTGCTGAGTTGCCGACCGGTTCCT
GGGGAAGACAGATCTAGGAAAGGTCTGTGGACTTCCCACTGGGAGGACTCTCACTGAAGCCTGCTTGCACTGGGAGGACTCTCACTG
AAGCTCGCTTGCTGTTTTGCTTGGTGCTGACTCTGGTGATGTTAGGGAGCACTGAGATGACAGCCTATGGTTCAGGGGTGTGCAGGC
TTAGTAGCTTGGAGGCAAGCAGACTACTGTGAGTACCACACTGGCTCCTCCTCCTTAAGGCTGTGCTGTGAATCTGAGTGTCTGACT
TCTCAGACATCGTGGGGCTTGAGTCTGGCTTTCAGGGCCTCACGAGGCAGAAGCGGGAGAGTGTTCTGAGCTTGAAGGGACAGTGGG
TGGGGTGGGTTGGGTGGGGTTGGTGGGCTCCAAATCAGAGAGTGCCAGGTGTTCTTCGGGAGGAAAAAAAAAGGAAGCAGTTTTCTC
AGGGAGAGATAAAGAACAGACTTGAACGGGACAGGAGGTCCCCATTCACTGGGTCTTTGCATACCAGGCAGAGGGCTTTGCACGCTT
GTTAGAAGGCAGGGGAAACATGGCAGTGGTGTGATTAAGCTTTGGCACAGAGAAGAGTAGTAGGAAGAAGCTGGCAGCATGAAGTGA
GGGCTTGAACCAAGCTAGGGACAGTGGAAAGGAAAAGGTGGGAACCCACAAGAGAGCAATGTGGGGCATCCTCCTTTCGGCCTGGGA
TGACGTATGCTGTCTGTGAGGGTAGATACAAGAACTGTGGCTTAAGTGCCTCTCTACATTGATCAAAATCCAGCCCTTCCTTGGAGG
GTAGACAGCAGTGAGCAGCCAGGCAGCCTGCTGTCCCAGAGAGTAGGGTGGCGGAGTGCCCATACCAGCCGGCACCAACTGGCACCG
ACACCCTGGCTGGGGGCTTCTCCTTCTGGAACCCATCAGCCCTGGCCTCCAGCCAGACCTGATGGAGACGGATACACCCTCTTCCTG
CATGTTTTCAAAGATATGAGTGGGGTAGTGACAGCATGCAGAGCTAGCCCCTCTAGGCTTCATGTAGTAGCCTGACTATTTTGTGAC
TATGCGTAAGGAGCAGGTGGGCAACTATCGGGGCACTTTGGGCCTCAGTTTCTGGGAGTTTTCTGAGTGCACTGGTGCTCAATCCAT
GCAGTGATTGTAAGAGAGGATTCAAGAACACAGCCGGGAGGTTCTTGGCTCAGCCACAGTCACTATCTTTAATTGACAGGAAGTGGT
GATGGACTGGACAGGATGTATAGGAGAGATGAGCTATGGTTACCACTGAAGGAGATAAGTTGGACAAAGTGGGGTTGGCAGTTCTCC
TAGGCCATAGCTCTGATGGGCTCGGGTGGGGGGGCGGGGGGGATTGCAGGAGGCTACCCAGTGGCAGCCTGGGGTCGTCCCACCTCG
TGATTCACAGATGTTTTTTTTTTTTTCAAAGATTTATTTATTATTGTATCTAAGTACATTGTAGCTGTCTTCAGATGCACCAAAAGA
GGGTGTCAGATCTCATTACGGATGGTTGTGAGCCACCATGTGGTTGCTGGGATTTGAACTCAACACCTTCGNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNCTCCAGCCCCAGATGGTTTTTATTATCTACCTTAGGACCACCTTTTCAACACCACCAGCATCCCTACGAGCCAC
CTCAGTGCAGTGACCAGAGAGGTCAGTGTGTTCAGCCGATCAGCTTAGCCTCTGGTCTTGTCTGTGTTCCTGGAGACTTTCCTGGGC
TCACAGTGCCTCCCTCCATCGCGATGCCCCTCAGAGGCTAGCTGGATCTCTTTCCAGCGTTTCAGTTAGGGCCTGGCTTAGTTCAGG
CTCCAGAGAAGGAGTACTGCCTGGACTGGCATGTTTGTACTGATGTTTGCTGTCCTCTGTCCACTCTGTGTGTGAGCTGGACAGACA
TGAAGGCTCCCCAGGGGCAGCGTCCTGCATAGCACAGGATCGTGATGCGGCTATCTGGAGTCCTCAGGAAGACTCCAGGAGATCAAT
GATGTGCTGACACCAGCACACAGAGAGAGGTTGCTTGCAGGTGTAAGGGAACAAGGCTGAGGTGGGCACAGAATGGCAGAGGTGGGT
GAGTGTGAGACAGGCCTAAGGGGAGGCCCACAGCAGAGAATCTGTGATAAGAGGCTGGGCAGACCTGGGTCAGCCATGTGAGAGCAG
AAAGAGTGTTCTCCAGGGTCTTCACTAGCTGCCTGAGCGTGCAGGTGGAGGTGACGGCTGTGGGTGTGTGGGGGGGGGGGAGAGGCT
GGGCTTGGGGATGCTCCAGTGTTTTCATCACAGCAAGTGACAAGGTGTGGTTGTGTCAACAGCCTCAGTGACTGGCAGACTAATCAG
CATCCATTCTTCCTTCCGTGGGTACAGCAGTCCCCAAGGGCAGTCCCCAATGCCTGGATTACCTCCAACAGTGGCAGAAGAAGCAGA
GCTCTATCTCCTGCTGTACCCCTGTCCCATGTGACTTATGGCTGTCTGCTTCTCCTAGAATTTGATCTAACCTTGAGGAAACTAGCC
CAGGATCTATACGAATGCAAACACCAAGACTACTCTTGAGTCGTGGTCCAGGAGGCCCAGATGAACCCTCCCAGGACTGCTTAGATT
TGGGAGCCAGGCGACCAAGAAAGGGCAGAAGGCCTCTGTGAAGTAAAGGCAGAGGAATAGGTGGGCAAAGGAGGAAGAGCAGACTGC
AGCCAGGGGCTCAAGCAGGGGAGGCACTGGCTGGGCCCTGGGTTTAGGCAGAGGGGAGCATGTCTTATCTATCTCATCGGGCCTGGC
TAAAAGCCAGTGCTGATGAATTCCAGAAGACTTTCTAGACAGTGAGTTCTGGAGCTAGATCTGGACAGCTTTGTCTTCCAACTGGGA
AGCAAAGGAGCCAAGACTTTCCTCTGGGCTTGAGGATTGTAGAGCACCAGGGAAAGGCAAGGTTTACGTTTTCCTGCCCTAAATAAC
AAGGGTGTCCCTGAGGGTCACTGTGAGCACTTTAGGGGATTGTCCTGCAGTTATTCAAACAAGGTTGCTTGGAGGGGTGTGTGTGTG
TGTGTGTGTGTGTGTGTGTGCGTGCGCGCTGGGGGCAGAGTTTGGCTCCCTTTCTGGATGAACCCTTTGGAGGGCCTTGTTCCAGAA
TACCTAGGCCAGAATCATTTTGGAAATGTCTCTGTTGCAACTGAATGTGGACACTGGTTAAAGAGGCTCGTGGTCCTGTGTAGTAGG
GCTCCTGCAGTAACAGCAGAGACTTCCTTGGTCCATGTGATCCCTTCCAAAGGCAGGTGAGTCCCATTCCTTCAGAACCCATTTGGA
ACTGGTCAGCAAACTAAGAGGTGTTGTATGGGACTCCTGAGGCTGAGCAACCAGGTTGGGCATAGCCTACAAGCTCTCCTCAGAAAT
GTATGGCCTTTGGCACCGCATTGGCTGGGTACTGAAGAGTTGACAGGATGTAGAGTAGGCACAGCAAAGAGGTTTTATCTGGGTGTT
AATTCTGAATGGTTGGTACCAGTTGCCAGAAGAGCTGTGATGAGAAGGATGGTGAGCTGCTATCTGAGCTCAGTGAGAAAAGTGCTG
TGATTGATTGATGATGTCTATCATGGCAAGGAGAGATGGAGGGAAGAGGGGATTTGCTTTCACTGGTATGGCAGAAGGAATGCAGAG
ATCCTAGATTGACCATTAGCGAATAACTCATATGTCTTTGCATCCCAGTTTCCTTAGCCTTAAAGTAGGAGTGATAAGGTTATCGGG
TATTGAGTAAGATAACGGAATTAGACATTTTATTCAAGCGAAGACACAGAATGCGGGTGTCATTGTTTTGGACTCACTCAGGAGCCC
AAAGTGACTTCATAGCTGTAGTGAAGGCTTTCTTTCTGCTTGCTGGAGGAACCACAGAACAATGTGTAAGACAGTTGATTGGCATGA
CCTTTGACCAGGCCTGTGCCAGGCATCTGGGACCATGATACGTTCTTGACTAAAGTGTCAAAGAAGTGTTTGCTTATTTTTCTGGTT
GGTAGTCACGTAATTTCTGTCAACGATTCCCCAAATGAAGATTTAAACACTGTTTCTGTGTGTAATGAGAGAAGGCCAGATAGCTCT
CTCTAGAACTTTGTCTCTGATTCTCCATTAAAGGTAGCAAAATTGCTTTATAAAGTCTGAAAGCCTGACAGGATCATGTATTAACAA
TGCATACATTATGCAAATGTGGGTGTAGTTACTTAATTTGCATAATAGATGGGCTGAAGGAGGGCTGTAAAGGGGAAAGGGAAGGAG
TGAGGGTACTTGTCCAGATCTGTGTATATTAGGGTTCCCCAGTCACCCCACAGGGTGCGTTTCCCCCTTCCCATGCTCCTACTCAGC
CTTGAGGTTACTCTGTGTCTTTCCCTGCTGGCTAGCTGCCTGTGCTTCTGTGTCCATCCTTCACCCAGGCAGGCGCAGGGGCCCTCA
CCCTTGCTCCTTGAGCAGTGAGGCTTCATTAACTCTCACAGTCCCTCTGGGGACAGACAGCTTTCCCCAAGAGCTTCCTCGGGTCAT
TGCGTGTCCTATATCTGTCCTGCTGAGGCGCTGGACTTTGTTAAGTGACATCTTGAATTGTATGTGAGTCTTGTCATTGGGTAACTT
TTTCTTGGGCTCTGTGAAGCAGAGGAGGGGAGGTATTTTAGGGGAGCGTACCTCCTGATCCATCTACATTCTCCCTGGACATATCTG
ACAGCTGCTTTGAAAGGTTTTTGTGCTCTGGCTATAAGGGGTTTCAAAGTTCTGTTTCTCCACCCTACTTCCCGAAACCCCTTTTAG
CCCTCCTCTGTCCTATACTCCCAGGGGCTGGGACTCAAACCCAGGGTCTTCTGCATGCTAGGCAAACTGAGCCCCAACACTAACACC
CACCCTTCCCCTGAGTTTGACAAGAACTGAAACTGAGACCAAGGGTATTTGAGGTCAGAAACCCAATGGATACTCCCTGGAAGCCTA
GATTTGGAGGGTTCAGGTTTAAGGGGTGCTGGGAACCATCTGTGTAGGTCAGACAGATGTGGCAAGCAGCTGGCTAGCTGGCTTCTC
TGGATGGTACAGTGGGTGTTCTGTGCAGTTGATAGACACAAGAGCCCTGCCCCCCTCTGGCAGAGGGTGGACAGGGGGCTAAGTCTG
CAAACAGTCTAGAGCTTGGTGTGGTCATACCTGCTCAAAGTTCCCAGTATTAGGGCCCATCCAAGGCTCTGAGACCCTGAAGATAAA
CAAGAACTTCCAGCTTTCCTTGGCTGAATTCCTACTGAGAGTCCTGGTATTTCATAAAGTCACTGGGTTTATTGTCCCCATTTCATA
GCAGAGTATGATAAGGCTTGGTCTCATAGCCAGGGAGCACCCAGGAGATCTTGTGGAGATCTAAGGGAGATCTTGTGACCCTCCCCC
TCCACGGCCTCTTCTCTTGAGACTGGAGTTTGACCTGGACCTCTGGTGCTGGGGGTGTGAACAGAGGGTCCAGGCAGTGTGATAGAA
GTAAGGAAAGGGACTAGAACTGGAGTCACAAGAGTCAGGAATATGAAGGACAGTAGAGAAGGCCCTGAATGAGGCATGTCAGGGAGT
CTGAGTTGACCTGGTCTTTGGCGGCAGTTCCTGGGTGGGGGTGATGTGGGCTGGCACCAGAGGGTGAGGGAAGGTGGGTAGGGACAC
TGAGCAGGGTAATTCCAGAGATTGACAGACCCTTTCTGCCCAGATATTGGGGTGCTGCCAGGAGAGGTAGACCTGGTATATTGGCTC
CTGTCCTGTTCACAGGGCCGTGGCTGGGGCAGGTGCAAAGTGTGGCTTCTCCCATTGGCCTGTTTGTTCAGCTGGTGCACTGGCATC
TCCTGCCTGGGGTTGGGGGCATCCGAACAGTTTGTGGACCCTTTCTGTCTGGAATGAGGTTACAGCTCTTTCCATACCCTCACACCC
GACGACGGACCCCAGAAGCACAGTGGAAGATGGCGCCAGACAGAGTCTCATAGACAAGGCCCAAGGGGAATCTGAGGCCGAGAGGGG
AAAGTGACTGTCTTTATCTTCGGTAGCCAATGGAGTGATAATCATAGGGGTTCTGGCTCACAGGTCACAGGAGCCTCTCCTCACTTG
CTCCACTTTGGGTTTGCAACATTGCCTCCTGGGAACATGAATGCTCTTGTATATATATAATATATAATATATAATATATAATATATA
ATATATAATATATAATATATAATATATAATATATAATATATAATATATAATATATAATATATAATATATAATATATAATATATAATA
TATAATATATAATATATAATATATAATATATAATATATAATATATAATATATAATATATAATATATAATATATAATATATAATATAT
AATATATAATATATAATATATAATATATAATATATAATATATAATATATAATAATAATATATAATATATAATATATAATATATAATA
TATAATATATAATATATAATATATAATAAATAATATATAATATATAATATATAATATATAATATATAATATATAATATATAATATAT
AATATATAATATATAATATATAATATATAATATATAAATGTGTATATATATAATATAATGTGTATATATATAATATATATATTATAT
ATATATTATATATATAAACACCTCTTCATCCTCTTGGGTTAGGCGGGACTTTGTTCTGGAGTGCTCTCACACGGTATCGCCAGCGGA
TGGAGCAGAGTCTACTGATCCTACATCTGTCAGGTGGGGCTGTGCACACTCACACAGTTTGTTCTCCCAGCCCTGCACGGACCCTTG
TGCTCCCTCTCGGTGCCCAGGCTTCTGACCCTGGAGGGGCTCTGGTTAATGTCGGCGGTGGGCAGTAGGGAGGAGCACCTGGCATGC
AGATCTGAGAGGCTGGCTGAAAGTGATGGCTGTTTATAGACTGTGGGACTTTAGGTGGGCAAATTCGCCTGTTAATTTTCTCATGAA
TGAAATAGGGGATAGTAACACATTCTTTGGGGGGTACACCAGCCCTGCTACATTTTTTCTGAGACAGGGTCTTTACAGCCTTGGGTG
GCTTCAAACTCTATGTAGACCAGGCTGGCCTTGAACTCACAGAGATCCTCCTGCCTCTGCCTGCCAAGTACTGGGGTTAAAGTTGTT
TGCTATCTTGTCCAACCCCTGTTATATTTTGGAATTACCTTGGGATCTTTTTTTTTAAACTGATGTAAAGAAAAGAAACGACAACAA
CAGCACCCCACGCCTGTCCTGTCCCAGCTACCTACCTTCTGACCTCATTGGTCAGGACTACAAGCCTGGGTACCAGGGAATTTCCCA
AGCTCCATGGGTTATTCTAATGTGCTGCAAAGTTTGGGAACCACTCTGTGGGTTTTGGGGGATTAAAGAAGATGATGCAGGTTCCTG
GGGAGAACTCAATAAATGGTGGTAATTAGGATTTTCCTCATTGCATTTAATGTGGGCTCTGATAGGAGAGGGGGGAACGGAGGGAGG
GTTTCGACTTTATTCCACTAAGAGTTTGTCCTCAGTGGCTAGGCTTTCCCTAGAAGACTTGTTCCCTCATTTCTAACACTATTTCTG
TCACTTTAGAGCCAGTTTTAGCCTTCCTCATAGCCATCATGGTTATGATTAGGTTGGGTGCCCAGACGGGAGCTGGGGTGTGCTGGA
CCCAAAGATAAGATGTCAGGTCTCTCCTGAGTCTCCTCCTGGTGCTTTTGAGTCCCCAGGATGGCTAATGGAATCAGAAGCTCAGGA
TGCTTGCTGTATCTTTGAAATGCTCTCCGCTGTTGCCTGGCACCAGCTGAGGCTGTGAAGGAAGACACATTTCCCCAGACTCCTCTG
CTGTCCTGACAGGAAGTTGTGCATCAATGGGCTGACAGGAAGAAAATGGCTCTGCTGCTGGGAGGATCTCATGGGGGCTGAGGGAAA
ACGTGGAATCTTCTGGTCCACACAACAAGGCCCCTCTTTCCTCAGGCAATTTCCCCGTTTATTTTCCTCGCAGTGACAAAGATTGGG
TTTAGCTAATGAGGAGCCGTGAGGGGAGAGTGAGGGGGGGGGGGCGAACCTGTGCAGGTGCGCCAGGTGGGAAGCGCTGTGGCAAGC
TGCCCTCGCTTCAGTCAGTGCCATTTCTCCCAGCCCCCTGCCTGCGGGGACTGCACGATGACTAAGCATTCTATGATCTCAAATGCT
TTTTCCTCTGTCTACCAACCACACCTTTGCAGACGCCTTTGATTTCTACTTCTTTTTACCAGCCCCCAAACCCCACAGTGAATCTTT
TAATAGCATAGGCCTTTTGGATCTCTAACCAATCAGTTGCCAAATCCTATCCAAAGTACACCCAGAATAGATTCTTTTAATAAAATA
GATCACATTTAAGCCAGCATAAACCTGTGAAATTTGTACATTTCTTCTGGAGCACCTCTTCAAATTTTACAATATTGAAATCACCAC
CCAGTGACCTTTCAGGAAATACTAGATAAATTGCAAGTGGCAAATAGACAAACTAAATGGACTAAGCACCGTTTCTAGCAAGTGCCT
TTCCTGGTGGGCTCTTAGTCCCAGTCTTTGAAATCCCTGACCCCCAATCCCCTACCCCATCCAAGCCCAGAGATCTACCCTGTTCTA
CCTTGCAGGCCTGCTTGCTGCTTTGCTCCTGGCAAAGAGCTGATGGAGTGGTGCAGCTCTGAGAAACAGTACTGGTATGTGGCTCAC
GTGGCTCGCAGTATGTGGCTCCGCAGCACCTTGTCCAGTCTGTGTTTGTCTCTGTGCTCTATCATTCAAACCCACTTCCACCCATGG
TCAAAGCCCCCAGGGTGCTCTGGTGGGTGGCTGAGATCTGCTTTCCTAGGCAATGGGAGGGGGTCCTTGGCTTTGCCAAACATGAGC
TGGGCTGTGGTTTGAGCTGGGTGATCTTCTTGATGTGGAGACACTCCGTGCCTAGGCTTGGTTACCTGAGTGTGATGGCACACTCTG
TGTAAGCTGTCATTTGTACATGATGCTGTCATTTGTATCCCCGGGTCTAGCCTCGGCTATGATTCTGCTGCATGGGTATCATCTTGC
TTGCCAGCTGTTGGTGGACAGGGTGTGTGGTGCAGGAATAGCATCCTGTTTGTGTTTAATGTAGGGAAAGCCATTTTGGAAGGCAAT
GATCTATAAAACCATCACCTCAGTGGAAACCCTTAGAGGGTCCTGGTCATTCAGTGTGCCTGTGAGTCAATGTGTCTCTTGGGCAGG
GGCAGGCGCCATTGTGCTCTGCTTCCTAAAGGCTGTTAATTGCAGTTGACTCTATGGTCATGGTCATGCGTTTATGGGCTCATTTCA
TGGATAAGCAAGCTCAGTCACTAGCCTGACTTCTTGGCCATCACCACTTGATGACCCGTGTCAGCACCAGTAGGAGCTGATCTTCCG
GCCACTTGGATTTCCACCACTATTTCCCTGTGACTCCTCACACAGTTCAGTTGTACCTGAAACCCCACTTGTCTCCCATGTAGGGAG
GGCAGAGAGAAAATTACAGCATGTCCACAGCAGTCTGGCCTTTCTGTAGATGAAGCAGCATCCTGAAGGCAAAGAAGTCTGGGTTCA
CTTCCTTCTTATTTTCCCCCCTCATATCTCAGGCAAAATGCTGGGTTTCTGGAGGAAGTAGAGAGACCCGAAGATAACACACTGTGT
GTGTGTGTGTGTGTGTGTGTGTAAGTGTGTGATGTATGTTGGGGGTTTCTGATTGTCAGATCCCATGATGTGAGTGTGAGCTCTGGG
GCTGTAGACGTAGGCAGGGCTTGCACCCTGCAGTACCTATCCCTGTCCCAGACTCTGGGGAAGTGGCCAAAGTCTCAAGAATGTGTG
TGTGTGTGTGTGTGTGTGTGTGTATGTGTGGTGTGCAGCTTGTATCATGCAATGTGCAAAGAACTTGCATGGTGCAACAGAAGGAGC
CCCGCGGTGCAGATCTAAGATCTAGCCACTAGGTCCTGTTTCTAGATCTGGATTCTGCAGCGAGATGACTGTGACCCAGGCCTCAGT
GTCCCGGCATGTGAGGAGTCCGAGGCCTGGCCCTGCCCTAGGATTCCCGGCCTCTCCCTCTTTGGTGGCATCTCTTTGTTTTGTATT
TGTCTCCACCCCTTGCCATCTGACTCTGTCTCCTGGCTTTACCATAACCAGCAGCTGGCAGTGGCTGGAGCCAGAGGGATGGCTCCT
CTAGGAGGAATGTACTCTCCTGACTCTGCGGACCATTGGCTTCCTCCCTCTGACCCCAGCACACTGGGCTCCCACCTTCCCAGCCTG
GGCCCAGCTGGGTTGCTTCAGTGAAGTAACCACTTCTCCTGCTTACAACACTCCACCTGGTCTGAAAGTGACAAGAGACAGCTGCTT
GGTAGTCAGTGACGTCTTTGTGGAGAAATGACAGTGCATTTGGCCTGCACTGTCTTTTTCCTTTGCTGCCTTGGGTTTTAAAACTCA
GCTGTTGTTATCTACACTGGTTGTCTGGGAACTCCAGGGTCGGCTCTTCCTGCCCAGGGAATAACTTTTGAAAGGCTCATTGGCCCC
GTGCTCCCTATCCCATCTAAGTGTCCCTCCGGGGCAAGGAGCCTCGGGAGGGCTTGGCAAGGCTCTCTGTTTTCCTTGGTCTTTGAT
CACAGCTGTTAGAGTTAAAAGCTTAAATAATGCATGTTAAACAAATAAGTATCTGTTTAATCAGTGCGATGGGGAAATTAAGGTGAC
TCAGGCTTTAAATTCCTAAGTGTCACTACCTGAAAGATGGCCTAAGTGGTAGAGTAATGGTCTACTCCTCTCTCCTTGGGAATTCAT
TCATTCTCCATCTAGCATTTATTGTGCATGTGCTAAGTCTTATGTAAGGCAAGGGGACAGAAGGACAGATGAGTCCCCCTCCCTCTT
ACCTCTGGTGGCCAGAGGAGAAGACCAGGGCCTGTGGGCCTTCCGGGCTCTAGCAGGCTCCTCTGGACTTCACTCTTTAGTTGGCAA
GCTTAAAGCTTGTTCCTGGAAAACAGAAATAGTGCAAAAGGGAAGAAGTGTGGGTGAGGAGCATGAGTCGAAACTGGAGGAGAGAGA
TGGGAGAATCTAGCAAACTTTCCCGTGAGGGAATGCTCCTCGCAGCGCCACAGTTCCCTGTTGAGACCCTGGCCTTGGGCAGCAGGA
GGCTGAGGAAGGACAGGTCCCTGTTCATTCTCTGCGCTGCATGGCCTGAGTTTTATCATGCTTATTTTTCCAAGACAAGGTTTGTCT
AAGTAGCTCTGGCTGTCCTGGGGCTCACTATGTAGATGAGCCTTGAGCTCAAGGTGAGGCACCTACCTGCCTGTCTTCCTAGTGCTG
GGATTAAAGATGTGAGCCACCATACTTGGCTAGTTTCATCATCTGTAAAGGCAGGTTTTGATGAGATGCTACAAAGGCCTGCTTAGA
TCTACACTTAGAGAGAGGCGTGGGGCTGGTGGACCAAATAGAAGCCACTCAACTATAAAATGATTTTCCTTGGCTTTTCCCATTTTG
ACACTAGGAGACAGCTTGTGGGGCCAACGAGCACCCCATTACCTCCTCCAGTCGCACTGCTGGAAGTTGGAAAGTTCTGGGTCTCTG
TGTTTGCATATTTTTCCGATTTGGACATGCTCTTCTTAGAGTTTTAGGAATTGTTTTCTTTCTGTCAGTGAGGCCCCTTGAAGCTTT
CAGGGCAAAAGGCTCTTGAGTTGGGCACACATTCCCTGATGCATTTGCCTAGCTTTTTGTCAGGCCTGACACCAGGTGCCCGTGGTC
ATCCCAAGTACCCAACCATGGGGCTACTTCAAAGCATAGAGAGAGCCCCCAGTACAGCTCATGCTTATGGGTCTATGGGGGAAGACT
TGACAATGTTTTGGCAACATTGTGTTGGAAGAACTGGACTTCTCACCTTGTGGCTCTAATTGCTTAAATATGCTGGGCCTCAGCTTT
TCTGTCCATGAGATGGGAGCAACTTGCTCTATCTCCTAAGGATAAAATAGGAAATTGAGAACAATAAAACCAAACAAAACCCATAAA
TAGGAAAATTCTAAGTCGATGTCTAGGACTCACTGATTTGATATCAAATATATTCTGATAGTTAGCCTTTGACAGGTTACTATCTCT
CTCTGTTCTGAGAAACTCAAGCCAGCAAGGGTTAAAGCAAGCTAGGCTTATGACTATGACTGTCCATAATTGATGTGATAAACCTTA
AAGGAGTGACATATAAGTGCAATTAATCCAGGCACAGTGACACTGCCTTGCTAAAAATAGTGTCTACTAATAATTACTTTGATCTAG
CAAATGATGCCTGTCTGCTAGGCTGGCTGGGGCAGGACGGGTGGAGGGTCTGCCAGTGCAGTAGAAAACTCTACTCCTCTCTTCTGC
AAGGCGAGAGGCTTCAGGGTGGAGACGCAGGGAAGGGGAGCCCATCTCTCAGGCCCTCTGGCAGCAAACAGTGTGGACGAGAAGGTA
TCTGACACACCATTTAGTATCATACACTGTTACTATTCATATTAATAATTATGCCAGACACAAACTTGACTTTCTGGGTTAGCAGGG
AGTATGGACGACCTGTGTATGGGCTGTACTGTGAGCTCATGGGATGTCTGGTCTTTTTCAATCCTGGAAGGAAGAAGAGATTTGCCG
TCACTTAATTGATATTAAAGATGGTAACAGTGCCAAATGGTCCACCTGCAATTTTTGGACTGGCTCCCTAGGTGGCTGGCCTCATGA
AGGTAGAGGCAGCTGGCTGGGATCACGGCGCTCTGTGGCCATGGAAACAGATGGAGCACAGAGGGAGGGATCAGTCAACCTGGCTTC
TGTGGAAGGGGAGCAGGTGTTAGCAAGAGCAGAGTGGCCTTAGCAGGGCTCTGTCTGGGCTCCTACAATCTTGGACATGGAATTAGA
AAGCTTGAGGTTAGTTGGGCCTCTGATCTGACACTCAGGATAGGCAGACCCTGTGTGTCTGGGGCCAGGCTACACAGTGAGTTACAG
GCCAGCCAGGGTTACCTAGAGAGATCCTGGGAGAAGAGGGTTAGGAGAGAGGGGAGTGGGGAGGAAGGCGGGGTGAGGGGGTGGGTG
GAGAGAGCTTGAGGTTAGAGCCTAGCTCCTTCACCACCCGAGTAGCCTTGGGAAAGCTTATGAACAAGCTCTCTACACTTGTTTTCT
CCATTTTCAAGTGGCAACACAAATTCCATTAAAAAAGAAAAAGAAAGAAAGAAACAGCCGGGCGGTGGTGGCACACGCCTTTAATCC
CAGCACTTGGGAGGCAGAGGCAGGTGGATTTCTGAGTTCAAGGCCAGCCTGGTCTCTATAGAGTGAGTTCCAGGACAGCCAGGACTA
CACAGAGAAACCCTGTCTCAAAAAACAAAACAAAACAAAAAATAAAATAAATAAAAAGACAACCAGCTCCTCTGGGTGGTGGTGGCG
CACACCTTTAATCTCAACACCCAGGAGGCAGAGGCAGATGTATCTCTGTGAGTTTGAGGCCAGTCTGGTCTATAGAGTAAGTTCTAG
GACAGCCAGGGCTACATAGAGAAACCCTGTCTCCAAAAACCAAAGCCCAAACCAACCAACCAACCAACTGACCGACCAACCAAACGA
AAAAACAAAACCCCAAGAAACAAACCAGCTAGCCCCAGGATCTGCTGGGCAGCAGTTCCTCAGGAGATTCTTGAGTGTTTAAGCTTG
GAAGGTTCCCAGGAGCAACCTCCACGGGGCCCCCAAAGCTGTGGACAATCTACTGTTGCTAGACAGGGACACGCTCACATTGTGGTT
TCTTTTAGGGTCGTTTCATACCCATTTGCCGCGCCACATTTAGTGCGCATGCGCACTGATGTGTAGGCCACAGCTTCCAAAGCACAG
ACTCTTCACCCTCGTTCTATTTCTGGACCGTGGAATTGAGACAGTCTCTGTTCTTCCAGCCTAGACAGCTTTGTTGACCCTTGTTTC
ACTAGTGTACATGTCGGGACCCAGTTCCCTTCATAGCAGATTCTTGAATGTCCCAGAGCGTGTTCCTTGGATGTCTGGGGAATGCTT
CTTAAAGCCGATCATGGATGCACTCACGAATGTGGGCGATGTCTGCTTGGTTCATCACCGTGATTAAAGATCAGGAACGGCTTGTCA
CCGTTCTTTGGGAAAGCCATATTTTCAGGTCCCAGCTGGAAAAGAAGCGTGCAGGGGATTGTAGGGCGGAAATCCGCTGCCTGCAAC
TCCCTGATCTCCAAAGAGGCGGGTGACCGACCATGTGACCTGTAGCTAGCCTTCTGGGCCCTCAGATAAGCTTGCTTGATCTGGTTG
GGTGACATGGCTCCCGCAGTGCCCCATTGCCGGCAGTCATCGCAGTCAAAGCAAAAGGTAGGCAGGCTGGCAACAGTTTGGTGCCAG
AGGACTAGAGAAGGTAGCATGAGTTTATAGAGTTTATAAAGGTACCATGTTCAAGCCTATTAATAGCCCATGTTTGGCCTCCGCTGC
TCTGTCAACATCATCTATCACATTCAAACAATTGAAGAGCCCATGCCAAATGGTTAGGAGTTAACAACTCACGGCTGGGCTGAGTTT
CTGAAAACAGAATTTCCACTTCTTATGCAGACTGAAATGCCAATTGTTCCACGCCGACTGGTTATTTTTCCAGACCATTTCCCTTTA
TTTATGTTTAAGCGGTTGTGTTTTTAAGTGAAAGGAAGATGGAGAACCTGGAGTAGGTAATTGTTATTTCTCTTGATTTGCCCTTGA
ATGAGCCATCAGAGAAATAGTCCTTTTCCAACCTTGTGATGAGAACACTCAGAAGCTGGGAGGAGTCAGACAGGACCCTGCCCTTTA
GGGGGCGTGTTCAAGATAGATGCAGGAAGGACGGGGGCAGGGAGAAATGGCAGCCCAAGCTTTGGGAGAAGGGGACTCTTGTCCCTT
TCTGTTTTGAATGTGACCTTGGGTGGGAGGGAGCTCTTTTGGGGCCTCAGTCTCCTTATCTGTAAAATAGGGAAGCTGGCTTAGCGG
ATCTGAGGCCAGTGGCTGATGGCAGGTGTGTGCTCTGCAGTACTTTTAGACACTACAAGACTCACGAAATTTCTGACTGGCTGAGGA
AACTCACTGGCCACAGCAGAGGAAGGAGAAGGACCAGGAGGAATAGATTGACTGCTGGAGAAAGCAAACGACTAGGTGACAGGTCTG
GAGGTGGTTTCTCTGACAGGGATTCAGGCTTTTGCAGAGCGGTTGAATTTAAGAATGGCTGGATGGACCACCTGATGAGCCTGGCAG
CTCAAACCTGGATTTGAAGGTTGCCCTAACACAGGCTGATTTCTGCTGGGGACAAACCAGCCATCCTGTCCTCTGTGTCCTTACTTC
CAGACCTGGCATGACTGTGTCCTGAGAAGGGGAACTGTCATAGACCCCTATGAGGCAATGGTCGGAGCATCAGCTAAGCACTACACC
TAAGAAAACTGAGATCAGATTTCTGAGAAAGTTGGGGCCTTCCCAGGTGTCTCCCAAAGGCTGTCTGGAGAGGAAGTGAGCCCTCAG
TCTCTGAAACCATCAGTCAGACAAAGCCTAGATGACAACTGCAGCAACTCTCACACAGGGAGCAGGGGGGATTTGGCTGTGCGGCTT
CAATTTATTTATTTATTTATTTATTTATTTATTTATTTATTTACTTACTTACTTACTTTATTTACATTTCAAATGTTATCCATTTTC
CTGGTTTCCCCTCCGAAAGTCCCCTATCCCATCCTCTCTACCCCTGCTCACCAACCCACCCACTCCCGCTTCCCTGTCCTGGCATTC
CCCTACACTGGGGCATTGAGCCTTCATAGGACCAAGGGCCTCTCCTCCCATTGATGTCTGACAATGCCCATCCTCTGCTACATATGT
GGCTGGAGCCATGAGTCCCACCATGTTGTCTCTTTGGTTGGTGGTTTAGTCCCTGGGAGCTCTGGGGGTTCTGGTTGGTTGAAATTG
TTGTTCTTCAACCCCTTCAGCCCCTTCCTTCCTTTCTCTAGCTCCTCCATTTGGGACCCTGTGCTCAGTCCAATGGTTGGCTTCGAG
CATCTGCCTCTGTATTTGTCAGGCTCTGGCAGAGCTTCTCAGGAGACAGCTATATCAGGCTCCTGTCTGCAAGCACTTGTCGGCATC
CACTATAGTGTCTAGGTTTGGTAACTGTATATGGGATGGATCCCCAAGTGGGGTAGTCTCTGGATGGCCTTTCCTTCAATCTCTGCT
CCACACTTTGTCTCTGTATCTCCTCCCATGGGAGATGCCACCAATGTCAAGATTCAGGAACTTCATTATTTATGGGTACGACCATGT
GTCTGGTGATATCTCCGTGATGATGTTCATTCCTTCCTCCTGGGGGTTCTTTCTTAAACCCCAAAATAGAAGCTCTCACCTCTAACC
TGTATATCATAATTGGAGAACTTTTTAAAAAGTCGGCAGAGATTCTGAGTTCATTGTTTTGGGTGGGGCCAAGGGCACTTAATCCTA
GGACCTGGGTGATTGTACCTGGTGGAGTTGCCACCCACATCCAGACTCACTGTCGGGATGAAGAGACACAGCTTAGCACGATGGAGC
TCATTTGCTTGTGTTTATCTCGTGTCCGGAAGGAGCCTTGGTGGCTCCCTTTGCAGCAGCAATCGAGCTTTAGGAAAGCCCTAACCT
GTTGTGTCTGCCTCCCACACGACACAGTGCACAACACATGGCACGCACACAATACTAGTTACAACCCCGACTGATGGTCGGCGATCT
AAGTGTGCCAGGAGTTACATGACAAGCAGCACGCTGCAGAGATTTCCTGCTTTGACAGCTCCTTTCTGCCGAGACCTTGTAAAGGAA
ATCATCTCCTTAATCAGAATTGTGTTTGCAGCCGAAGGGGCAACTCATGAGACTTGATATTTGGAGAGCTTGGTCTCCAAAGTGACC
TTCTCTTGCTTTCCTTGGGGTACCACAGTGGGAGAGTCAGGCCTGGGGAGGATCGCTGCCTCTTAATTGATGTTCTGGCCATCATGC
TGGCTCCACCAGACAATGCTTGGCCACCCTCTGCTCAGAGCTGAATGTCCTTTCTATTTTCACTACATGATGTCCCTCAGACATCAG
GTCCTATGTCCTTCTGGAAGTGACAAAGTAGAAGGCCACAGAGAAATGCCCTAGGCCAAACTGATTTTATGTACTTTAGTCCAAGAG
GCTGTCTTCCTTAACTCGCTAGCATTTCCTGTCTTTCCTCCCCCATGCCAGCCTAGGCCCGGGCTAGACTTGCAGGAACATCCCACA
CCGCGAGCCCTGACCTCAAGCTGTCATTGTTAACATGCCTCAAAAGCACACAACTGGGACCTGGCTGTGAGATTTCCCTCCAGACAC
TATGCTATGAGAGCACAAGTCTTAAGAGTCAGGCCTGACTGCCCAGGTTCAAATCTTACCCCATTCCTTCCCTGTCTAAGGACTGTG
GGTGAGAGACAAGCTTCTTCTCAGCTTCAGAACCCTCGCTGTAGTGTGGGACCACATAGCAAGCCAGTATGGGACCCATGGCAAGTG
ACCTTTTGATCACCCTTGCCCCCTGCTTCTTGAATTTGCAAATCCAGGACAGAAGTGTCTACCTGTTGGCACATCTCTAAATTTTGT
TCCCTTCTCTGAAATAGTATTCAGCATTCAGCTGATATTTATGGACCTACTAAGCACTGGACAGCATTTCACATGTGATGACATAGG
AATGAATTTATTAAGAGATGCTACCCTCTATGGCTGATACTCTGGCTGGGACAAGCAGTGGTGAGGGTAGTACTGACTGGGAAGAGA
TTCCTGTGCAGCCCCAGGGAGTGTGGGACCAGATCAGGTCCCTGGGATCATCTTAGGCAGGTAATGCTCCCAGCTGAGAAGATGAGT
GAGTCAGGAGGTAGAGAAGGTGGTTGAAGAGGGCACAGCAAGGAGGGTTCCAGGAAGCTCTCTGCCCACGATTAAGGTCAGACCCCC
CCCCCTCCTAAGCTGGAAAGCCTGCACATAATGGCCCCGCTGAGCCTAGGACTGAGAGGAGAGAGTGTTCCATTTGAGGCCTTCTGC
CAGGGCACTATGCCTCAGATGCACCCCAGGAGTGGGAAAGCTCAGAACAGATGTGCTACATATAAAGGAAGAGAGCCACATGCACGG
GCCACTGTGAGGTTGAAGCCCCTCTGTGGCCACTGGAATCTTAGACAAATGGTCTCAGGAAAGGTGTCTGAGGCTTAGAGCCTAATC
AGGCTGACCCACACCCTCAGAGGTTACACCCTGGAGGAAGGGAATGCCCTCTAAAATCTCATTGCCTATGGATAAGGCTGCGGGCTG
CAGCTCATTCACGCCCAAGATGGAGGCGAGGGCTGTTCAGACACCTGCTAGTGCCACCTCTCCTGATTCTAGTCACTCCCTGATGAC
TAAGGCCCAAAGGAACCAGGTCTTCTCTCAATTGGAGCTTCCTGCCCTCCAGACCCCCCCTGCTAGTTGCCTCTGTCTCTTCTGACA
TTTGGCTGTGGGCGGGACGAGTGTGGAACACGATGGAACAGGGCTCAGCTGGTGGGCAGGATTCAGTCTCAGTATACTGTGACAACA
GTGTTCTGAGGCTCAAGGCAGGGCTCAGCCCAGAGTTTGGGCGAAGCAAATGCTCAGTAGTCTGTCTGGTTGAGTGAAGGAGCGTGT
ATGTGGCCTAGTGACAACCCAAGGCATCTCTGAAGTACCAAGGACAGGGGTGCTGATAGTGGAGCTTGTCACTGGGACCGATTCGTT
TTTCTCTGCTCTGGGTGGCAAGAGGAAGTGCATTTGACTTAAAAAATATTGTGGAAAGATAACCTTTTCTTTTTTCACTTGTATCTC
GTGTATGTGAAGGAGAGCAGAGGAACTGGAGACCCTGAGAGGTTAAGGCCCAAGGAAGGAGCTGGAAGCCCAGTGTCTCAAAGGCTT
GACTTGAACCCTAGTCTTTTGTTGTCAAGTTCTATGTTATAGAAAGACACAATAAAAACCTTCTCATTAGCTCCCACACTAACAACA
GAAACCACTCTTTGCTCTGTTGAGAAGACAGAATGAATGGGATCTGGAGCTGGCCGTTTCAGACCCACAACTTAATTCCTTACAAAA
GAAATGTTTACAAAAGGAGTGTTCCTATGACCCGTATGTGTCCTGGCCTATAGACTGATGATATAGGCCATCGAGCACACATAGCTA
AATGTCAGGGTTTTGAATTCCAGAACCTTCTTTCCTTGGAGGTCTGAGGTATGCACAAGGCCAGGCATTTAACTTGCTGTAGCAGTG
ACTACTGGTCACTCTACAGCGAGTCTAGAGCTGACTTCCAAGCCCTCTTCCTGATGAAGTCCCCAGGGTCTTAGGTTCTGTTTCTCA
GATCTGTGCAATGCCCAGCGACCATCCCTACCCACTGTGATGGTGGCATCAGATTCCCCTGCTCTCAGGAGGGCAGGATCGAGTGGG
CTGAGCCTTCCCATGATTCCATTCTGGACCATGAATACCTACAATGGGAGGGGACATCATGCCAAGGATGCTGGTAGGCCCAGCATG
ACCTCAGGTGATTTCATGACCCTGACGACCTCAGGCAGCTACACCCAGGAGGAAGACCAGCTAAGGTCTGATCGGGCAAGGTAGTGA
AAGTTCAGAAGGCAAAGTCAGGAGAAACGTGCAGATGGGAGCCAGGGAAAAGGAAGGCTAGGCTAGGTGCAGAACCAAAGCCCAGGT
AGGGAAGAGAAGCCGGGTGTCCCTGGAGATCAGCAGCCAGGAACGCTGACTTCCCTGCCAGGGGACTGGGGGTGGGGAATCAGGGAA
GGAAGGCCTGGCTTAATCTGCATAGCATTAATTAGCAAGAGTGTCTAATTAGGACAACCATTACAGAGCTACAGTACGAAGCCAGTG
CTGAATATCAATCAGTCTTGGAGCGGTTCCATGAAATTGAAGCAAAGGTTGACCTTATTAAACAGAAAAGATTTCTAACTCAATTAT
TTCCCCAAGTCGAATGCACCAACCGAGGTGAAGTCAGGAAGGCGCATCAGACCTTAACTCGGGAAATACAGGTTCTGGATGCTATCA
CCTTTCTCCCTTGAGGGCTTCTAACGAGTGACAGGAGTCATGAATGGGCCATGTGCGTGCACTGCGTGCTCACATGCACAGCCCTGG
AGCTCATTACCTTGGCTGGGGGAGGAGCCTGCGCATCCTTGTGCTGGGGAAGCTTATGTTGACTTTGGCTGGTGCCCTTACCTGACT
CCGTGCATCATCTTCTGCCTGCCCCCCCTTCTCAATGTTCCAAACAATAAAACACATTAAAAACTTCAAAGTCTCTGGGAAGATCTA
CTGTAATCATTACAATTCTGGCCTGTCTCTGGAAGCTACCAGGCAGGCCAGGAGAGCAATTTATACCGCTGGCTCAGAGGCCTGGGA
ACCACTCCATCACCCTGTGCACAGCCCCAGAAAGGGACCCAGCAGCCGTGAGCAGCTTCTCTTTGAATCTGGAGGCCAGTGGGCTCA
GCCTCAGGGCAGCAGGCTCACAGCTTGAGAGAGGTATAGGTGGCAACATGGCTCCCTCAAACCTCCTGACTTTGCCATTTTTTTTTT
TTTTTTGGCTATGCCCTTTGACAAGTTAGGCTTCTTGGAGCCTCAGTTTCTCTGTCCCCGAAAGAGAGTCCATGATGCCTCCCTTGC
ATGGTAGGTGCCTGTAGCAGCAGCAGCAGCAGCAGCAGCAGCAATGCTTTGTTCTAGATTACTATCAGTGGGTAATCACAGACCCGA
GAAGGGCTGCCTCAGGAGCAGGACAGGGACCACACTGTAGGAACATCTCCAGGAATGTGTCTTGGTCTCCTACCCAGCCATTCGTCT
CTTGTGCAGCCCAAGGCTCCACTGAGGTAACAAGATACACAGTACCTGGGACACTGAAGGGCAGGGTAAGTAAAATCTGGCAAACTA
AAATCCGAGTTCGTCGCATGACCTTGGAGAAGTCACTTTAACAACTCTCCAACTAGTATCGTTTTATCAGTAAAATGGAAACATTAG
GTTCTACCAGAGGTGAGATAAAATGCGTGCTAGGTGTGTGGCTCGAGGTAGCACATGATCAGAGTTCAAAGAAGATTAGCTATAAAG
TGCTTACTAAGCGCCTCTTAGATTCAGGGCATCAAATAGGTGAGGGGACCATATAGTATAAGTGTGTTATGTTCTGGTCCTCTGCAA
AAACAGGTTGCACTCTTAACGGCTGGATCACCTCTCTGGTACTCTATCGGGAGTCGTTTTTATATTTGTCTAGTCCTGTGAATCCAT
GGAGCAATAAGACTGGTCTGAAGCGGGTGCGAGTCCCTAAGCCCAGCCCAGGGACTCAGAGGCCGAAGCTCCCAGCTGGTGTCAGTT
CGAATTTCCCTGCTCAGGCTGAGAATAGTTACAATTCCAGCCTGACACTAGGAAACGGGCGTGAGTAAGTGAAACGGAGTCTCAAAG
CCCAGAGAAGCTTCGGTCGTTCATCCCCATTGCAGGGATGGACCTGAGGCTTGCATGGGCTCCTGCTGTCACCCGTGGGTGGTAGAG
TGCATGTGACACGAAAAGCTCGATCTCTCCACTGCACAAAAGCCTCTTACTGGCAGCGCAGGGCTCAGATTTTGAAAAATAAACTTG
TCAATAACAAAATGCTATGTCCTTATTAATAATTTGAGGAGAGAAGGAACATGGTAAGAAAATGCCCTACAATGCTGAATATTGATA
TTTTCCACAATCTTTTTCTACATGGGAAATTTCAAAAACAATCAGAACCAGAACGGTTTGGAGTTCAAAGCTTTCCAGTTAATATTC
CGTCACAATTTCTCTCCATGTCTTCAGATACTCTCTGTCTTTAGGGATGTACTAGTGTCCATCATTGGGTGTCCTTAGATGGTCTGA
GCCGCTTGCACATCCATGACCCTGTAGGACTAGACGGATATAAAAACGACTCCCGATAGAGTGCCACAGAGGTGGCCCAGCCGTTAA
GAGTGCAACCTGTTTTTGCAGAGGACCAGAATGTGGCTCCCAGCACCCACATTGGGAGATTTACAACTGCCTGTAACTCTAGCCGGG
GATCTGACTCCCTCTTCCAGACTCCGTGGGTGCTGCACTCACATGCACCCCTCTTACATTCAGATTCTAAGACTAAAAGTAAAATCT
TCACATATAAAAATGCCCCTGTGTATTCTTCGTGCTGGGGATTATTTCTTTGTTTTTAAAAATGCAAGGGAAAAGGGTTGATCAGTT
GGTCATGGGACTCCTCCCAAGCCATCGAGTCAATTTTAAGGCCCCAGATGGAGCAGGTTGGGGCGCAGCAGGTATGGCAGGTGCCCC
ACCATGTGGTGTTTGGTGACTAATTAGTCAGCCTGTGTCATTGGTGTCACCCGGAGAGTCATGTCAGTGGAGTTCAGGTGGAAAAGA
AGATAATTCAACATTTTAGCGAATAGAAAAATGTTCTAGATCCAGTTAGACTTCCTCGACTTATTTCCCAGGGGATGAGATCTCAAG
GCTGTATGTAACTCTCCATTAGTCCAGTGTAGCTGTGTCTCTCTGCAGGCTGCTGCTGGCCCAGCAGGCTGCACCAGGCAAAACCAC
TGAGCAGCTGGGAGCTGCCTTTTAATAGAAAAGCAACAACTTTGAGAAGAAATGGAAGAGGTTTAAAGGAGAGGAGACAGCGTCTGG
CCCCCATCCTTTATCTCTTTCCCTGAAAATGATGGAAACACACTTAGAGGCTGACTTTAGCTAATTTCTGCCAAGAACATGAAATGG
AGGTGGAAGTGTGCCACTGGTCACAAGCCATCCTGGAGTGGGCGTGATGCTCCCTACATGCCTCTGGGCCCCTCTGACAAGGACCTT
ATGCTCACTTCTGGCCGGAAAGCACTGTAAGCTGTCCCAGTTCTTTCCTGTAGAGAAATTCATCTGGTCCCACATCAACCTTGTGTC
CCTGAGCTTCCAGCCTCCCCCCACCCTCTTTTTTTTTCAGCCCCAGCTAGCTCTGGGATCAGCATCAAGTAAGCTTTCAAAGGTTGG
AACCTTCTGGAGGGTCAGAACTGTGTTCTATTTTCCTTGTTGCCCACATTGCATCAGAAAAGCACCAGGACTCCCCGAGACACCCTT
ATATACGTGAGCTCAAGGAGGTACTCTGGGTACTTAGCAATTCTGAAGGTCATGGAAGATGCTAAAATAGGCTCAGTGCTGGGAACG
GAACCTGGGACCTGTGTGGCTAGTACTCCTCTACCTCTGAGCTACACTACTACTGAGTTATACCCCTAGCCTTTGTCCGATTTTGGG
TAGTACAGAATCTACACATTCCTCCACAGCTAGATCATATAATGCAAGTAAGTGAGAGTTTGTGCAAGAAGACAAACTGAAAATCTC
TGGTGGGCGGGGCCCTCCTGGTGGGCGGGGCCGCTCTGTCTGTCCCCCAGCATGGACCACATGGTAGTGTATCCAATAGGTACTCAC
TCAGCAAGCAAGAACTGGTTGAAATCGTTGTAAAAATTAAATGAATTATAACTTGGGATGCTGAGGTAGGAAGATGGTGAGTTTATA
TAACCAGACCTTGTCTCAGAAAACAACAAAATGGGGTCTTGGGGAGATGGGGTCGCAGGTGAAAGCAGGTGCCACTGAAGCCCGATG
ATCTGAGTTCAGTTCCTGGACCTCACATGAAGGTGGAAGGGGAGAACTGACTTGCAAAGGTTGTCCGTTGGCTTCCACGTGAGTGCT
GTAGTGCATGCACATGCCTGCTGTTACCTGTGCACATCAACAGATGATACAAACCCAAACACACAAGGAACCAAACTGCGAGTGAAT
GAACGCATGAGTTATTTCACTGGTTCTCCCTTATGGCTCCAGGGCTAATTTGTGCCTGGCATAAAATTGGTCTCAATAGCCATATCA
AAGAAATGAATGCTGGAAGGGCCAGACACACCAGAGAGAGAGAGAGAGAGAGAGAGCAGCTCCCGTTCACAGTGGGTGGGGGAGAGT
GTCAGCATGGGCACCCGCAAGTCCTGGATTCAAGGATCAAGCTGTCCCCTTAACAGTAGCTGCCATATTGAACCATATGGCTCTGCA
GGTCCCAAATTCCTTTCACCTCTGCCTCAATTCTCCCATAGTATCATTTGGCAATTAATCATGTCTTGCTGTTGAAATCTCTTCCAT
TGTTATCTTAGTTGTTTTGGCTTGAGACATAGTATTTGTACCCCTTCATGGAAGTTTCATTAGTGGCTTGGAAACGTGCATTGGTGT
GTAATTATCATATCACCTTGATCAGCAACCCCCATCACCTCAAACTTTGTGCAGTTCTATGTGGTAAGAACCTTGGTCCTTTTCTAG
TTGTTTTTGCAATGCACCATAGTTGCTTATAACCATCGCTGCTCTAGAGAGAAAACTAGAGTTATAGTCTTTATCGCCTGATTGGGT
TTTGGTCTCTTGTCTTGATCTGCCATTTGGGTCTTGTCAGGAGCATTCCTGTCTCCTGGTGGAGAGTTGGCCCAGGGCAATGAGCAG
TGATCTGGCCTGGGGGTCTGAGGCCCTGGCTCTGAACTCTGGTGTAATATATTCACCCCCTCCCCCATGGCATTCTATACAGGTGGT
GGGGGGGAGCAGAAATTGGGCATTATTGATTAGCATTGTTTCTTTGGTCTTCCTTGGCTCTCAGACCATGTAGAGAGCCCACTTGGT
GGTCCTTGGAAATGCTGCCTGACCTCCCAACGGCCTTTGCTCAGGGCTCTTCCTGTGTTCCTTTATGACTGGAGATAGCTCTCCTTA
CAGACTCACCCCACTCAGGAGTGTGGCAAGCCCTAACTAGATGCTCTGATGGAAAGGGGCGAACAGTACTGACAGCCAGCACTCACT
GAGCCAAGCTGCTTCAGGTACCATTGATGGTCATGCAGCTCTGAGTGGCTGGGGCAGGTTCTGGACTCAGCAGACAGCCAAAGAGCT
TACGCACAGACTCTAGGCTCTCTAAGCCATCTGGGCTGAAGTTGCAGGGAGCATCCTCTGCCCTGAGCAGCAGGCCACCACAACCTT
CCCCTGCCCTGGACCTCTTTCTGCTCCTTTGCTGGCCCCTCATCCAGTGATTCTTCTGCTGTTCTCGTCTCCCTCTCCTGCCCTTGG
CTTGAGCCAGGGGACAGAGCAGCGACCAGGACATTCTGATTTATTTCTGATGGAGCTCTTCATGGCCAGAGACAAAAGCGACCTCAA
AATCTTCTGGATTGTGTTACGATAGAAACACAAGCTGTCATGGTACGGACACCCAAACCATATAATAGTGGCACAAAAAATAATTAC
GCCCATGCGACTTATGAGCGTGGAGGCAAGTATTCAACACAGCAGTAACTAGACTCTAGCCAGGTATCACATACATAAAACCTTGTG
CATAGTGGGGTCATTCTGGAAAAGCAAGGATGTTTTCTCCTTGAAAGCAAAGGCTCACAAGGGGCAGCCTGGGCTCAGGCTAACTGC
TCTACCCTGCTCCTAGGAGGAACTGGGGATGGTTTGTCCTCTGCCCTGCTGTTTCCTGTTATACCTCTTCCATGTGCTGTTTCCCGC
CTGGCTCCCTAACTCTCTTTCCCTCTCTCCTGCAGGATTCCATGTTTTCGTTGGCCCTGAAATGCCTTATCAGTTTATCCACCGTCA
TCCTGCTTGGTTTGATCATCGCCTATCACACAAGGGAAGTACAGGTATGTCACTCCCTGACTTCTAGTAGCAGCCTATCCTGCTGCC
TTGGTCTGGACTGAACCCACCCATCCCCAACCCAACTTGAGTGGCCCCACAAGATGGTGTTTGAAATCTTCTCTTTGGGTCCTCACG
GAGGGTGTCTTGAGGCTACCCTGTGACCCTCCTCTCGGGTGATCTCATAAATAGGCATAAGGCTTCATGAATCGTGGCTGTGTGGGG
GTGCACACATATATAGACACACACACACACACACACACACACACACACACACACAGTGCCTCAAACACCAGCAGGGAGCCTGCAAGT
TTTGAATGTGGAGACTCCTCCTCTGAAGCCACCAGGCTGGGATGTGCCTGTCATTTTCTAGCAAGAGAAGATGTAATGTGTGATGTG
GTTGAGGTAAGGCCTCTATTTCTGGCTGCCTAGGGTTGGTTCAGTACTCTGGGAAATGTTTCCTGGACATCTGGGTATTCACCCAGC
TTGGCTGGTTATATAGGGAGGTTGGTTCAACTTAAACTGTGAGACTGAGGCAGGATTGAGTGGACCCCGGCATCCTCTTGTGGCAGT
GAGTTGTCACCCTTCCCCCACCCCCGGGGCCTGCAGACTGATCTGCTCACTGCTGACCCAGGTCATCCAAGATATCCAGATGTTTCT
GATCATGTGTGTGTGAATCTGGATACTCCCCCCGCCCCCGCCCCCCATCTTCTGGTCAGAGGAGCATGGACAGAATTACATCTGGGC
AGCAGGAAGAGACTGACGAAGACCAGAACTCAGGAGTCAGAAAGACTGGGGTCAAGTCTTGGCCAGGACTTCTTCTTACTGTGTGTT
TTTGGCTGGTCCTGTGACCTTTCTGGGTCTCAGCCTCCTCACCTGGAACAAGGAAGCTTTATTCAACTTTGCTGGGCTCCTGTGAGG
GTAGAGTAAAAAGGTTGTGTGTATGTGTAAATGCTAGCTGGCATGCTGGTAAGCTTAGGTCCATTTGTCCTCATTGTCCTGACACAG
AGATATCATGGGGAGCCTCCCTGTCCCTGGGTCCAGCTTCTAGCCAAGTTCTGGAGTTCCTGTGGCTGGATGGGATGTGGTGGTTTT
TACTGTAGGTCTTCTTCTGGCCATAGGACTCTCAGTATGTCAGTGGCATAGGACACCCATTTAGAGGCTGGACTCTAGTGTTTTGCA
GTCTTGTGTACTTAGATTGGGCTATACTTTGAGGGCCGAGTACAGAGGTTACTAGGCAACTTTTTTCATAGCGTTCTAACTGGGCAA
AGCTTCTGGACTCAAACCCAGACCACCGTCCAGCAAATCTCTGAAAGGAGCCAGTAGTATAAGTGACGATGACCAAGGGACCATGCC
AGCATGGAACCTTCAGCATTGGCTTCATCCCTACCTGGACCCAGCTGGACGGGGTTCTGAGGCTCTTCAACCTCTGGGTGCCCTCAT
GTTTTCATCTGAGATGGGCTGTGTGGGAAGTTGTCATAGAGGCCAGGTTGAAAAAGAAGAGTCAAAACTCAGCTGGACCTGGATTGG
GAGGAAGGGCAGCGGGCAGTTTCTAGCACACGGAGGGCCTCCCAGACACCTCTCCTTTCTGCTCCAGACATTGTGAGGAAGGAGCAA
GGTCTCGGATTCTCTTAGGGTCAGGCCTCTTTCCTACTTCAGGTCCACAGACCTGACCTAGTTCAACTGCAAAGCCTCTGGTCTTGG
GGGACAGAGCCATGCTGCTGGCAGACCCTAGTTCACCCTCAGCTTAGGACCAGTCTGTTTCAGCCCTAACCACGCTCTCTGGGTAAC
TTACATTGTTGGCCCAGCACTCACAGTTTTAGAGAAACAAGGTGAAATGGGTGGCTTTGGTCTTTTTAATTAATATTATTTATTACT
GATATTGATTTAATTATATAATTGTTGTTATGTGTTGTTCTGGGATGGAGCACAGGCTCTGGGCTTATCTGGCCAGTGTTCTGCTTC
TGAGCTACACATCAGACCAAAGTAGTGGCTTTGACTCCTACCTCAGTCACTGCAGGCTGGATGACCTTGGATGATAATAAATCCTCA
GTTTCCCACCCAAAGAATGAGAGTCCCACCATCCATTCTGGTGAATTCTTAAGGGTGTGGGAATGGTACCGGGAACAGTATGGTTTC
TAGAAATGGAAACCTTACTCTCTTTCCTGTTCCACACACAGCTGTCCTCTAACTTCTAGCTGCATTTTGGAATATGCCAGGTGAGCC
AGCCTCATGGCCACTAGCACTGCTGCCCAGAACTGCAAGTGTCTGGCTCTCCTCCTGCTGTACCTAGGCTCGGTTTCCATGACGTAG
ACCCTCATTGTGCTGTACCCACTGACTCCCAGGGGAAGTGACAGTGTCTTCACCTTAACACTCGCATTCTCCCTACAGCTGTCTGGG
CTGTCTCTTGTCTCCTTGAAGGGAATCGGAATGGTTTAGCCCCAGAGAGCCAGCAAGAGAGGTGTTCCCATCTGCAATCACCTGGCC
AGACTACAGCTTTCTGCATGATGGCTTTCTCCATCACTTCAGAAACAAAGAAACACAGATGTCGTTAATATTTGCATTTCCAAAAAT
ATACAATGCCTGACAGAGAGATGGGATAGAAGGTACTGGACTAAAGAACTCTTGTTTCTAAAACTAGTGACATCATGATTTTGCGTG
TGGTCCTGAGCCTCAGTTTCCTTGTCTAATTATTAGGGTCCTGGTTTTAAGTTATCCAGTCTAGGTGTGGCTGCTCATCTTTCAGGG
TCCTGGTTACTGAGTGTTTAGTGTGACTAACAGAGAGTTTCCAGGGGGAAGCAATGGAAATGCCATCCTCAGCGCAGGTCAGCCACT
GCTGGGGTGAGCAGGGTGGGAAGGCTGTGGCTGTGCCATCTCCTAAGGCCACCTGCCTAGCACAGGGCCTGCTGGGCTCTCTGAGTG
CTACAGTAACGGCCTGGGGAGGGTCACCTACCAGTGACAGCATGCATTTAGAACACACCCCATTCTGCAGAGAGGAACTTGCTCTCA
GCCCCACTCCTGTCTCTGGAGCATCAAGGACAGAGCCAGGTTGCCATGTGGTCACTTCTGTTGTTGAAGATGCTCACCCCGCCTCTC
CTCAGCCTTCATCTTTTGTGGCATTTTTAGAGTGGAGCTTTGAAAGATTCAGGTACCTTTTATCCCCAGGCCAGTGTATTCTGAGAA
GAAAGGAAAATGCAAAAGCTATGCAATTTGGACCTTCGCTAAAATGTAGCGGGAACCCATACTTGTTCATAGACAATGCACTCCTGT
GTCATTATGTTTCAGATGCCCTTGGTGCACCCCTGCTTAATGCAGAAGGCACTGGGCTTGGCTGCCCTCCTGATAGTTTGCTGTCTC
TTCTCCACTGCCCATCCAGCTCCATTATTTATTCAGCAGGGGTTTAGTGAGGTCACTCTGTGCACAGCCAGTGAGGAGCTCTTATCC
CTAGAAACTCTGACATCACCATCGCTTCTCTGAGGGGTTGCCAGCCTCAACTGTGTCTGATGCTGTCCTTGAAAGCAGGTTTTCTTT
TTCTTTCTTTCCTTCTTTCTTTTTTTAAAAAAAAGATTATTGTTTCTATGTATATTGTTTATTTATTGTTATATGTAAGTACACTGT
AGCGGTCTTCAGACACACCAGAAGAGGGCGTCAGATCTCATTACAGATGGTTGTAAGCCACCATGTGGTTGCTGGGATTTGAACTCA
GGAGAAAGCAGGTTTCTGATAACATGGATGTCAGAGCAGCCCAGCTTGGGTAGAGGCCAGGCTCTTTCACCTTTTAGGGTAAAGATA
TATGGCAGGTGACCTTTAGGGTTTTTTGTTGTTGTTGTTTTAAACTCTCCTTTACTCTATTTCCTTCTTGAAAAATCAGATTAGTGG
TAGTCCCTACCACAAGGACTGTTGTGAAGAACAAATTTGGGCATTACGAAAATGCTTCAGCTCAGATAAGGGCCTATATGTCTTGCC
CACAGCATGGGAAGGCTGTTTGTCTCAGTCACTGTTCTATGGCTGTGAAGAGACACCAAGACCAAGGCAACTCTTATAAAAGCATTC
AAATGGGGTCTTGCTTACAGTTTCAGAGGCTTATATACTCCATTATCATCAGGGTGGGGAGCTCGGCAGCTCACAAGGCACTGGAGC
AGTAGCTGATCTGAGGGTGTGTGTGAAAGAGAGAGAGAGAGGGAGAGAGAGAGAGAGAGAGAGAGAGAGATGCCTTGCATGGGCTTT
TGAAAACTCAAAGCCCATGCTAGATGACAAACTTCCTCCAACAACTCACTCCATCAAGGCCATACCTTAGACAAGGCCACACCCACT
CCAACAAGGCCACACCTACTCTAACAAGGCCACACCTCTTAGTTCTTCTTAAATAGTGCCTCTTCCTGATGACTAAGCATTCAAATA
TATGGGCTTATGGGGGGCATTCTTATTCAAACTCCACAATATCCCAAATAGAAGTCATAGAATAATCAGAAGCCCAGGAGTGGAAAC
TTCAGTGTCTTGTTCAGAATGTCTCCAGGCTAAGGTCTCAGACAGTGCTGGGGGGCGGACAGTGATCTGGGGGTGGGGTGGGGTGGT
GGTGCTCCATTGTGGAAGGTGGGCAGGCCCAGTGGCACCTGTTTGTCCTGCTAAGGAGCTTTGAAAGGCAGTAGCAGGAATCACAGA
AGACTGCCAAGCCGGAGGACATTTACATTTTAGACATGCTCTCTGCTTGCGAGTCTTAGAAGAGTCAATTTTAGAGGCAGGAAGATA
GTTAGAAGCCGATTACAACATCCAGGTGAAGGACAGTGATGCCTGACCAGGATGAGTACAGGAATGGGGACAGAGGAGGGAACATGT
GTTAGAGAGGCTGAGAGAATGGAAACGACAAGACTTGGTTACCAATTAGGGTGGTTACTTTCTTGGCAGGGAGAGAGAAGTCTCCCA
ATTGCTTTGGATAACTCAGTGCTGGAGAATGCCATTCATTAAAACTGAAAACACAGAAGGAGGTCAAGGTGGGACGGGTGTTGGGTT
CTACTCTGGCTTGGGGGGGGGGAGCTCTGGCTGCCTCCAGCGTGGGAATGGGAGCTAATGATGTCGAGTAGTTAGGGATGGGTGTGC
AGGTGCAGTGAGATAAAAGAAGGGCGCAGGGCAGAGACCCCAGAGGAACATTGACAGGTGATAAGTAAGTGGGGGAGGAGCTGCTAG
GAGACTGATAAAGATTACAAAGAGGGAGGACAGGAAGCAGCCTTTGCATCTGCAGTGTGGAGGGCAGGGCTGAAGAACACACTGTCC
TCGCCCTAACTTATTCCTTGTCATGACAGTCACTGGGAATGGCAGTGTAGGTGAGGGGGCAGACTGAATAGGTGGCGGCAGGGAACA
TAGACTGTTGGTGGGCTACCAGCAAGGAGCCAGCTGAAAGGGAGAGGGAAAACCAGGGGTTTACCTTCTGATCCAGGAGCCTGCAAG
AGAGGAAGGGTAGAGAGCCCCAGGGAGGAGGTGCTGGCTTAGGCTTGGGGTGTGGACAGCAGAGAGAATTCACATCACGAAGCCCCT
GGAGAGAAGCTGCAAGCACCATCAGTTACAACTTGTGTTATTTCATTAGCTGACTCAGGGGGACAGGGCTTATCTACTCCCTGTCTG
GATCTCACGGAAGCCTCTGCACACTTCTTTAGTTTCCTCTAATGTGACAGAAACATAACGATAGTGATAAGATATGAGTCTTCAACT
TTATGAGTTTGGAACCCAAGGGACCCAAACTGGAGATTTTCATTTATTAGGTATATAAAAGTGGATATTTTTTATTAAATACCAATA
GGGTTTTCAGAAGTCAAGGAACTTGTTTAAAATTATAGGCCATAAATTATAGTCAAGAGACAGCTCACATTTGTAAACAGCTCACCC
ACTGTGGTGCTGTTGTCCAGGAGATCCAAGCAGCCTCCTGGGTGATCACAATGTACTGTCACCAAGACACGATGCCTGTGATACAGG
AGGTGTCGCTGGTGCAGACTCTTTGGGGTCCTTTTGCTAAATAGGTTAGTGGCATCTTCTCATCAGGGGACAGGCAGACAGTCCAGT
CTTTCTCTTTGCCTCCCTCTTCCTAGAGAGAAGTGAACTCAAATCACATTCTGAACTTAGAAGGTGACCTGGCCTTAAGAGGAAGGG
TGTGACAGCTGCGTTCTGAACTGATGTCTCTAAATTAGCGAGTTTACCCACATGTGATTTATAACACTGAGCCAGAATGCCCATCTA
TTTACTTAACGCTCAGAGCTCATTCTACACTCCTGCCTCTGGGATGTGATTGACCACCACCTTGAGCCAGCTGACCCATCGTCTTAA
GCACCATAAGAGGAGGCATGAGTCCACGTGTCAGGAAGGAAGAAGCAGTGGGGCATGTCTCCTAGAGGGGGTAGGTCAGTTTCCTAG
GGAGGTGAACCTGGGCGCTGGAATCACTTGGAGCAGGGACCTTATGTGTAAGAGACAGAGCTGGTCTGGAATTACACAAGAAGAGGA
GGAAGAAGTTATGCCTTTCTTCCATGGGAGCTAGAGGCTGTCCGTCCTTTCTGCAGATACACCATAGGCAGTCCTGAGGGTGGAATT
CCTGGATGCTTGATTGATGCATTTGAAGTACTAGTTCTGGGGCTGAGGAGATGTCCCAGTAAAGTGCTTGCCAGCCAACCATGTGGA
CCTCATTTTAGGTCACCAGCACCTACGCAAAAACTGGGCTTGGTGTGTTTGCAACCTCTGAGGGGTGCATATGTGTATGTATATGTG
CGTGTTTCTCTGTGTCTGTGTGTAGAAAGGTAGATCTCTAGAGTTCACTGCCAGTCAAGTTAGGAAAATTTAAAAGCTCCAGTTTTA
GTGAGACACTGTTTCAAAGAATAAGGCGGAGAAGGACTAAGAAAGATAGCTGTCTTAGTCTACTGTGGTGATAAGATGCCATGATCA
ACAGCACCTTGGAGAGGAAAGGATTCATTTTGTTCTACAGCTTATAGTTCATCATCCGGGGAGGTCAGGACAGAGGCTTAAGGTAGG
AATCTGGGTGCAAGAGCTAATGCAGATGTCATGGAAGAATACTGTTTATGAGTCCGCTCAGCCTGATTTCTTATAACATCCAGGACT
ATCCATCCAGGGGTGGCACCACCCTCTGTACGATGGGCACATTAATCAAGAAAAATGCACCACTGGCTTGCCCTTGGGCTAGACTTG
TCGGGGAATAGTTTTCATTGAGGTTTCTTCTTCCAAAATGACTCTAGCTTGTGTCAAGTTGACACAAAAAGTACAAAACCTAATGTT
GATCTCTGACCTTCACATGCATGTGAACACACATGCACACACACAAGCACACGCACAGGCACAGGCACACACGCACACACACAACAC
ATGAAATGAATTCCGGCTCTGAGTTCCTGAGATACGAAGAGCCTAGTTTCTCAGCATGGCTTGATCTCAGGGTGTCAGTTCAATCCC
AGTGTGGTCAGCACAGAGGAGAGCAGGGCTCAGGTGGTTCTCAGGGGAATCCAGCCTGACAAGGACATTCTGTGTGGCAGCCTCTTC
TGCCAAGTGTGTGTCCATCTCCTGTTCCACTTTCATTAACACTGCATTGATAGATCACAGCACACGTGAGAAACAGAAGATGGGAGA
GGGTGTTCCAGGGACTCAGCGCGGTGCTGTTTCTCTTGGGATCAGCTTGTGCTTGATAGGGCTGGAGAGAAGGAAGCAAGAGAGGAG
AGCCTGCCATGAAGACACCCACAAATGCCTGTACCCCAGCATTCAGGAGGAGGCAGGAAGATCAAGAGTTGGAGGCCTGAGCTACAG
TGTGAGATCATGTCTCAATAAAAGAAGAAAGGAGAGAGTGAGAGTGAGAAGGCTAGAGAGTGAGGAAGCGAGAGAGCGAGCAGCAAG
AATACAGATGCTTGGCTTTTGGCAGGGGACCAGTCCACCTTCTTTGAAGATAGGAGACTTAGTGGCTGGCACGTGGGCAGTGCCTGG
GTGGCGTTGGTTGCACTGTGCCTAGTTGGTGCTTTATAATCTGGGAGTGTCCTCACAGACCTGCCCCTTCACCATCTTCACTTTTAG
CCTGCAAATCCCTCCTCTAGGAACTGTCTCTGGATACCTCCAGTCTCCAAGCCAGGAGCCTGTCTAGGCTGTGACTTAGCATTAGCT
CTTTGCCTGCTGCGGCATGCCTGATTGGTAGATGGTAAATGCTAGCTTTAGTCAGCCCCACTGCTCTAGATGTGGGCTTCATGGTGA
TGGAGACTGTATTGGAGCTAAGAGATGTCTGTCTAGCACATAGTGAGACTCAGAAAGAGCGGCTGAACAGCGAGTGAGTGAACGGAA
CACAGATGCGGTCTTTCTTCCTTCTGTGGAGAAAACTCATCTCCAAAAGACTGGACTAGTCTCAGTCCACAGTGCTGGGTTCTGGCT
CGTGAGTTTTGTATCCCAAAAAACAAAGGTCTCAACAGGCTACTCTTGTGAATTGGTCTTGCCCACTGCTGCTCTCTGGTCCTCTCT
GCTGCACTCTGGTTAGCCTTTCTCACGCCCCGGCCATGAATCGCCATGGTTACTATATCAGCCCTGATGGTCAATTTTTATGTGACA
CTGTGTCTTTATTTTCTAAATATTTAAACACTCGAGCTTAACCCTTTTGCCTCATAGCTGGCTTCCTGCCTCTGTTCCACTCAGCAG
GCATTAAGATGCCTGCTCTGCCATGATATCAGGGGCGAGCGGAGGGCCAGGCTCTATCTTGGCTGTTGGTACTGATCTCCAGTTTTG
TCTCTGGGAAATGGGTATCCTGAATCATACTGTTTCCTAACAGAATGTGTGCCGTGCTTATTAAAGGTATGGAAACCCATATCCGGG
ACATGTTGAGACTTGTCCCAGCCAGGATAAACAGCTCTAGACTTCCCAAGTCCACTTGCTGTGTGCCATTGACGATGCCTCTTTAAT
TGTGGGAGAGGATGAATGAGGATGTGTGTGTGTGTGTGTGCTCTTTCACTTATATGTGGCCAGAGGTTGACAGTGGGTACCTTTCTC
TGTCCTTCTTTTGAGCCAGGGTCTCTCATCATTTCCCCTAAGCTGACTGGCCAGCAAATCTCCAGAACGCACGTGTTTTGTGGACTT
ACAAGTGCATATGGCCGCTACACCCAGCTTTTAGGTGGGAGCTGGGGATCTGAACCCAGGGATTCACGCTCGACGCTTGGACAGCAT
GCAGGATCCTCACAGAATCATCTCTCCAGCCCTGCAAATTCTTCCTGCCAACTCTTCCAAAATCTTCCCGGGCCCTGGGAGTCTCTG
TCCTAGATCCTGACGGGGCTCCGGGGCAAATTAGTTTGGGAGGAATTATGTTTGTAAGTGGAAGGTGACTTCTCCCTTTTCTTCTCC
CCTCACTGGCTTGAGACAGGCCCCAGCACCTGAATATAGGTTGCCTTGGAAACCACTCAGTTGTCTTCACCCTGGGCTATGTCAAAG
GAGTGAACATTGGGTGTGCTGTTCAGGAGGCTTCAGCAGTGTAAAGTGTCATCATACCACCAGGGGGACTGAGCTAGTGTGAAGGGC
CTCCTATCCGCCCCACCCCAGGGCTCCGGCTCCAGTGAGCTCTGTGCTCACAGGATGTAATAGCCTGTGTCTGGAGTATTGGTCACT
GTAGCCAGATAGGCCACCACTAGTTCCACTTTATTTTCTTTTATTTATTTTTTTGTGGTTGTAAAGTAGATAGATCAGGCCAAATGA
TCTCTGGGGCTTCTGATTCTGTGAAGGTACCACAGCTGAGGGACACTTGATGACTGGGAGATAAAAACCAGAAGGAAAGTGGCTGAG
AGCTTCAGAGGACAATGACAGACAGTGCGTGGTCAAGAGGCTCCTGGAGGGGCCTATTTGAACAACCTCATCTTTCTTTTAACAGCC
TCGCCTCTTTCTCGGAAGCAGAATTGGACACCTAGCTAATGGCCTCAAAGTTGAAATAGAAAGCCATCAAAATGACAGTTTTTTGGG
GGGCTCTGGTATCTTGTAGTTTAAAAGAGACTTCTCTCAAGTCTTTCCCAAAAACAAACTGACCGGCCTTCTTTAAAGCCTTGAGAA
TGATCACGGTATGTGCCCAAGCTCCCCCACCACGCAGTTCCAGCCTCCTTCTCCAAAGTCCCTGGGCAGCGAGCTCACATCATGAAA
AATCTGTCTCTGACTGTGGGGACTTGCACGTCTGAGGCTTTTTCAGAGTTAATTCATCCAAATTTGTATTGCTATGTTGTGTGTATT
TGTTTATTTCTTCATTTGAAGTAGCTGGGAACTTGTGGCTTTTCCTGCCTCAGCCTCCTAAGAGCTGACCTGATTTTCAGACGTGTG
CCACCATACCCAGTTATCTTCCTCCTCTGTTTATTTGGGGCTATTAATATAGGGGTCCCACTTAGGGCTGAGCACTCAGTATACATT
GATTCTCAGTATTATGACTAACTATTGATCTCTGCATTAACTGTGACCACTGCTCAGAGAGGATTCTTTGGCTAATGTTAAGCACAG
CACTACGCTATGGATCTAAGCATAGATATTTGGAAGATAGTTTGACAGCATGTCATTTAGCAAAACAACAGTAACAGGTTGTCCCCT
AGGGCCTGTGACCTCAGGACTCATGGACTTTGGACCAGCACCAGGCCCAGATTCTTTACTGTGAACCCAGTCTCAAATCTAACCCTA
AAGCAGCTGGTTACATCTACAACCTTTGTGCCACTATTGCACTTGTGGGCACATCTCATCCGGCAGATTGGTACTATAGCATGCAGG
GATGGCCTGGCTACCACCTTCTGGCATGATGAAAGTTAGTCAGCAGGGTGGGGGGATGTTTTCAAGTTTGCCTGATTTCACTATGTC
CTTCAATGAAAGTATGTTGTACCTTCTGCAATTGGGTTTTACAGTTTAGTTTTTCAGGGAGGAACAAAGAGCTATGGCAATAGCTCG
GATTGTATTAAGGGCCTATGGGACCTTCCTGACCCCATAGGGAAGTATCCTATACTTGGTACTGAGGTTTTTATTTTTATTTATTTA
TTTATTTATTTATTTGCCTTTTTTTTTTTTTTTCCTTCGAGACAGGGTTTCTCTGTGTAGCCCTGGTGGCTGTCCTGGAACTCACTC
TATAGAGACCAGGCTGGCCTTGAACTCAAAAATCTGCCTGCCTCTGCCTCCCAAGTGCTGGGATTAAAGGTGTGTGCCACCATCGCC
CAGCTTGAGGTTTTTATTTAGTAATCCATGACTCACAAGTCTGACTCTGCAGGGTATAGCTACCATATTTAAATGTTGATCTCTCTC
CACAATGGTGACAATCCTCCATCAGCAATGTGTATTAGCCCCACTGTACTGTGTCTGGCCTCCCACAGGCTATGAAACCCCACTTCC
CTAGGGGTATGCGCTTTGACAGCACCACCAAGGTTCTCTGCACACTCAGAGGAACAGTAGCCATCTCTGTAGTTGCTCCTTCAATGC
CTAATACACAGCAGGAGCTCGATGATTCTAAAGTGGGCGAGCAGTATCTGACTCTGAGGATTTGTAAGATGGGAAGACACTGCACAG
TTTTTACCGTCTAGAAATTTCTATACACTTGTTATTGCATTATGCTGCCAAGAAAATTCTGGTAATTCTAGTTTCCTGTTGGGCAAG
GAGCTAGTCAAGTGACATTTTCCCGTTTCTAAGAAGAAAGATGGCGAAATCGGCACTGCTCAGATCCTTCTAACACAGTAGTTGGAT
TAGGAGAAGACAGAACCCATCCTTCATTTCTATTTTGTTTAAAAGACTTGGTGCAAGTTAGAAGACATTCCATCACTCTGAAACTTC
CCCTGCCAGAGGGGAGGGACACCCATGGGCACTTGACCTGGGTCTAGGTTGGATTTTGTCATTTATTTCTTTTTTCTTTTATTCATT
TAATGTATATGAGTACACTGTAGCTGTCTTCAGACACACCAGAAGAGGGCATCAGATCCCATTACAGATGGTTGTGAGCCACCATGT
GGCTGCTGGGAATTGAACTCAGGACCTCTGGAAGAGGAGTCAGTGCTCTTAACCACTGAGCTATCTCTCCAGCCCTTAAATTTTTTT
TTCTTAAAAATGGTAGTAAAATATATGTAACATAAAAATTTGCCACTTTACAATACAGTTCAATGGCATTAAGGGATATTCATGTGT
GGTTCAAGGATTATCATCAGAATTGACTTCATTACCTTCTATGTAACCTATGTGCTTTGATACAAAATAGATTTGCTTCATGCCCTC
ATTTGCACAAGCATCCTTTATCATCCAGCTCCTGACCCCACCTCCATCCTGTGTAACACACTGTATCTCAGGCTGTGGTCCTGTTTC
AAGCCTTGGCCTGGCATGCAGCTGCACACACGGCGACAGTGGCAGCAAGCCACATGTCCATTCATCTCATGTGGGCGAGGGATTCCA
GTTATCCTTGACAGCAGTCTCATTTTACAGATAAGTGAAGTGAGGCTGAGAGGGGAGAGATTACCTATTCAAAGTCACGTGGCTGAT
GAGAGGCAGAGCTAGCCTCAAACAGACAGCCATCTCCAAATCCTGCAGACTTCTTTGCTAGTTGGGGTGCACTCCACTCTCCAACCC
ATCCGTGCCCCTGACTCCTGTGGTATCCAGAAGCACTCAGGAACACGGGTGGGTTGTGGGACTTAACATAGTGGAGAAGAGTGAACG
TGATGGGGTTCTGGGCTGAAAAGCAACGAACACAATTGGTCCTACCTTTCAAGCTCCCGAACACCTTGGCATCCCCATCTGCTAGAA
CCTCGTTCTCTTTCTGGTTTTCTTGCTGTCTTCCCCTTGGGCCCTACAGTTGTCTAAGAGAGGGGTCCAGTTTCCTGAGGGAAGAGA
GGTTACAGTGTTGGTGATATGCCTGAGTTTCCTTGCTGGCCCTCCCCTTTCTTGCACGTGTAGGTAGGTGCTGGCTTTTCTTGCACA
CTGGCTGCCTGGCTGAGCCCTATAGAGTCTTGATCTTGCTTGCCTTCCTGCCCCGCTTGGGCTGACACCCATTACTTGTGCCCTCCT
CATCTTGTCTGGAGTCACTAGCCCTGTTATTTCTTGCATTCTTATTTGCTTCAGCCCAAGGCCCTGAAGACTGGACAGTCTGGGGCC
TGCTTTAGAAGTTTTGTCTTTGCTTGCTATAATCCCACAGGCCAGGGTTGGTAGCCTTCTCAGGCTCTCTACTCAGTGACCATTCTT
ATGCTGTGCATCTGCAGTCTTATACCCAAACCTCCTGAGGGGGACCTAGACCTTCCCAGCACAGTGTTTTTCCAGATAGGTCTTGGG
GTCCTGTCCATCGCGGCTGCTCTGAACAGATCCCATTAGTGCTGCACTGGGTCCATGCACCACCCTAACCTGGACAGAAGGGGGCCA
ACTCCTGGTTTTCTCATTCTCCTGACTGTAGCAAAGATGGCTTCCACGGCAGCTCATTGGTTCATTCAAAGCATGCTGTCTTCATTG
AGCTGATGATCCTTCAGCTGTTCACCGGGTCCCTCATCCTGTCATCTCTGTTGTAGAGGCAGCTTTTGGGGAGGGGAAGGTGAGGGA
AAGCTGCCCCTTTGGTTGCCCCAGTGAGATTTCCCTTGAGTTTCTTTTATCTCATTCAGCAGCAACGTATTTCGAGCAGTCCCAGCT
TCCTGTTCTGTTCTTGGTGTAAAAGCCTTGTTCAAATCACTGAGACAGATGTGGAACAAGAAATTGAATCTGGGACAGTCGTTTTCT
TTTGTTGGACTAAACAGAGGGAATTGGGATTAAAAAAGAAGCCCAAATAGTTGCAAGCCTTTTATTCTAAAAATCAGTGGTCTCCCT
TAGGCTGCAGTATCCTGCCCCTGTCCGCCCCACCATTCCCTACACACCAGGCACACCTGTGAAACACTCATCTTCTGTAACCAATGC
TTACTGAGCTACAGTGCTTGCTTGGTGCTGGGAAAGCAAGCTGCGCCTGGGCTCTGAAATTGGGTGACACATGACACTTGACCATTT
GGTAAGACTGTGTTGTAACAATGTTGCTCCAACAGCCTGTGCTACAGGAGAGTGTTGACACAGAGCCGCATGCCCCGGGGCTCTAAT
ATTCCCAATTGCTCCTCCCCAGCAGCTGGGCTTGGCCCTTGCTGCTGACCTGTGACTAAGCTCCCTACAGCCACAAAGCAGGGATCA
GTAGCCAGCCACGGTGGAGCCGTCCGCCAGGTGCAGAGCAGCCTCAGCCTCCGAATTCAGACCATGAGGCAGCATCGCAGGAGTGAA
GGCATAACATCCAACGTCCCGGACACCTTTCTGCTCACGGACCCCACCCCTCGCTTCTCCCAGTTCCTTCTTGGGTGTCACTGGAGC
AGGAAGCTAAATGTTCCTGTCAGTTCTCTGCGGTACCACTTGTTAGAATGTACTAGAAGTGGTAGCCCTTTGAGAGCCAAGAGGAAT
CGCCTAGGCTGGGCGTGTGTAGGATGGAGGTGGGGAGGGGCAGGCCAGGCGATGTTTGCCTTCCCTCTGTTGTTAGAAATAGCAACC
GTCCTCCACCTTGCTGAAGGACTGGACAAAAGGTGATTAGTCCCCTAAGTAAGGGAGCTATTGAAGGTCTGTTCAGAGCTAAGCACC
ACAGGGGACAGCAAGGCTCTGGTCATGAACAGGCTTGTGATCTAATCTGAGATAAACATCTCCACTTGTAGGCACAGTGAACAGCAA
GGGGGTGTGGGGGCTTCCCACTTAGGTGGGAATAAGGACCTCACACTGGTGCTGAGGAGCAGGAGGACAGAGAAAGCTGGAGCAAGA
GAGTGTGGGGATGGGTGGGTCCTGTAGGGGAGCGAGCCTGCTGAGCAGCCAGGAGAGGTGGCTGGGAAGAGGGTTGTTTGTGTGTTT
GTTTTGTTTGGTTTTTTTTTTTTTTTTTGGTTGGCTTTTACCATGTGGTCACTTTTAACCAAGATTGGAGTTAAGTCACACGGGTAA
AACCCACTCTTTCTAACCCAGCAAAGATGGCTGTCAGCCATGTGGCAGCAGCTAAGACAGTGGCAAGCCTCCTAGTGCTTTGAGGAA
TGGAAAAGATACAGATAAGTGTAAAGGGACTAGAGAGAAGTGAGAATGGCTGGGGTCGGGTCATCCCGGTGAGTCTAAAATGGTGGC
AGCCAAAAACGAATGTCTCCAAGCTGCTGTGGCAGAGATTTTAGGAAAGAATGAGAGCTCACTCATAGCTTATCAAAGACCAACGGG
TGGCAGTGGCAGTGAGGTGAGGAGACAGACACACACGTGGGGGACATAGTTCCCGGGTCACAGGCGCCGGCCTGGATTTCCAAGTTC
AGGTCTGTGCTGGTTGAAAAGTCTCCCATGTTGGAGATCTCCAAGACCAGCTACCATATGCACTTGGTTGAGAATCAGGAATGACTA
GTCCTAGCCCTAGAATCCAGAGATTCCACATCCCATCAACAGAACACGCTCCCAGGGCAGGGCCCGCAGGAACCCCAGTTTCTCTCG
CACTGTCAGATCTTTTTGGATGGTTGGAAGGAGTAGCGTCCATCAGCTTTACAAGATTTTACAGGCTTGCTGTTCTCTTACTGTTTT
CAATTACCTCTACCTAAAAACACATCCGTCCCCAGCAGCTTTGGGGGTCTACCTTCTCACAAAGGCAGGGTTTTGAATGGGTTTCCT
GCCTTCCAGATTCAAACTTGATAGTCTGGCCAGGTGGCTGAGAACTGCCATTATCCCAGAGCGGCAGCGGCAGGGGCTCCTGTGCAG
CTGTGAGTCCCCCAAAGCCAGCATGGACCATCTTGTGTGTAGTGGGTTGAGAGAAGGTGTGTCCACGTTGGGGTCACGTAGGAGGAG
ATGTGCTTGCTGGGCCTGGGCAGGAACTCAGGGGCATGGCTGTGGCTGGCCCACAGAGCAGAGGCCTAGCTCACAGCCTGTTTCCAT
TTCTCAGCTTCTCCAGCCCTTTCACTAGTATTGCCCAGCCCCTTCCCTCCTTCTTTATGTTTTCTCTGTCTTTGACTCTCTTTTTGT
GACTGCTTCCATCTTTTCCTCTTTGTGTGTGTGTGTTGAAGCATGCGGTGTTTGTCACTGTGGGCTGACTCTGCGTCTGTGTGTGCT
TGTGTGTGAGTGTGGTCTTAAAGCTGGATTGACAAGGGATGGGTTTTACTTCCTGGCTCTGGAAAAAGAGAGGGAACGACAGTGAGG
GACAGGATCTCTGAGGCAGAGAACATTCCGATCTAGGTGTGCTTTGGGGGATGAATTTAGTTCTCAAGATGGCTGCGATGTTCTAAT
TGTGCAAGAGTCCTTGAGCACCCACAGTGGGGGGTGGCCATGTTGTCCCTCATCTGCTGGCTCAACTTCAGGGACCACAGGAAACCC
CAGGCTTCTAAATGTTATGCAGAGCTACAGTTCCAGCGGCCCTGAGCTGACAGAGATGGATGCCCAGCATTAGAGTCTCTCCAGGCC
CCTTTCTAGGTGCTCGCGGTTTTGGGAGCACTTCAGTTTTTATGGCTGGCATATCTCAAAGAGGAAATGCAGATTAATGGTGTGTTC
TGCCATGTTCTCATGTTCCTGGGACTAAATCAGTAAGATCTGAAATATTAATTGGGCTTTTATGAAGAGAGGATGAAGTACGGCGTG
GCATGGAATATTCTAGATTTAGGATCATGAGTTTGACTGTCAACCCTGCTGCTGCTGAGATTTGCTCTCTACTGAACTGCTAGCTTT
CTCCGAGTCTGTTTCTTTCCATGTATACCAGACTGGGCACACACCCGTGTTGTATCAGCTTAGAATATCACCAAGCCTGGCACAGCC
AGGGGCTTTTGGTGAGTATATTCTAATTGCCTCTTCCTTGATAAGACCGCCCTTAGCTCACGACACAGGATCCAGTTCTTGCCTGAT
GTGGGTACCTGCTGAAGCTTTTAATGGATTCAGCCCTTTGATGAGGTCTCTGAAGCCCTTGATGGCCATCCTCAGGGGCTTCTCATA
GCTGTGGTCAGCACACTCCTAGGGGCCTCCCTTCATCATCCTTCTTCTCAGTGTTGATGGCCAGCCTAGGGCTGAGGGGAGCTTCTT
ATCCCTCCTGTTAAAGATTGGCCAGTCCCATTGCCCGACTCAGGTCCCAGATACAAGCTTGTTGTCAGTACATGGGTCATGGAGCCC
TCAGTGCCCTAAGTACAGGGAACTGGTTCTCATTAAAGGCGCCTGGCTGGGTAAGATGGTGACAGTGAAACAAGGAAACATTGAGCA
CAGGGCCCCGTGATGCACAAACAGGATCTTATTTAACCTTCATATTATCCTCAATCTATTCCCATCTATTTTGTATTTGCGGCTGTA
GCTCGAGCCGCCACACATTTATTATCTTACACTTGGGGATTGGGGTCAGAAGTTCAAAATGGCGCCAGCTAAAGCCTAAGTGTTGGC
AGGGCTGCTGGGGTAATCCATTTGCTTGGCTTTTCCAGCTTCTGGAAGTGGCTTGCCTTCCATCTTCCAAGCCATTAGTGGCCTGGT
TGAGTTTTCTCATGCTGTGTCACACTGCTGCTGACTCTCCTACCTCCTGCACTGTATTTCAAGGGACCTTTGTGACAATATGGTGGC
CATCTGATAATGCAGGGAGAACTCCACACCCCACAGACCAACCATCTTATTGAGTCTGCATCCCCCATTCTTCTTTGCTGTGGTTTG
AGAACTGGATGTGAGTGTTTGAGGGATGCTGTTGTGATTCCCACGGCCCCCTTCTTGCAGTTGAGGGAAACCAAGGCATAGTTTTCT
CATGGGATATTAGCTCCCACGGCCAAGGTTATGTGGCTGCTAAGTGGCAGACTGGAAGTAGAGCGAGAATTGGTAGATCCAGGAAAG
TCTGAGAAACACACTAGTGCAGGCTAGCCCTCTGCCCATGGCAGTCAGAGGAGAGGGGACAGAAGGCGCCCTAGTTTGAACCCTAAC
TCGTCTTAGTCTAGAATGGGGAGGACAAACTGGCCTGGTGAGACACTGCAGGAGAGGCAGTGGTTCTTCAACTCGGCTGTACACTGG
AATCACCCTGGGAGCTTCCAAATGATACCAGAGCTTCGCCCCTAGAGAGCCGGGCTTAATTGCTCTGGGGAGACCCTGAGTTTAGGG
ATTTCCCAGGCTCTTCAGCTGATTCTGGAGTGTAATAGAATCATCACTTTCAACTAATCCACAGCCCTGGCTGTGCCTGTCACCCGA
GTAACTGCATCAGAGTCTCCTTAGAGTTGTCTGCAGCCCCCTCCCATTCTACTCGGGACCCTGAGCAGCAGACCCCTCTGCCCTGCT
CTGCCTCCGTGTAGCCAGGGCTGTAGGCTAGAGGACATCACGTGTACCCAGAGGAGCCAAACCTTTGTGATGAGCAGGTTCTTTTCT
GGGTCTCCCTGGTCTCTCTTTCAGGGCTGTCTGCACAACTGTCCACTTCCCCTTTGTTCATGTTAGCACACTACCCAGGTTCAAACC
TCAGCCCGTCAGGTCCTGATTTAGCTTGGACCTCTCTGTGCCTCAGTTCCCTCACCTATAAACTCAGGATGCTAATAATGCTGTCCT
GGTCAGTGTGAAGATCAATAGCTTTGCAGATGAGGGAGATTACAGCGAAATGTTTGCTAGTGTTACCCGTATTATCACCTAGCCCTG
TGGGCCCAACTGATTTCAACAAACATTGATTCTTTCTTTACTTAGTGCAAGGTTAGGAAAGGGTAAAGTGTCTTCCCTGCTCTGAGA
TAGCTCGCAGTGGAGGAAAAGGTAAGTCAGTCAGATCAGACAGCTAATTACAGTACAGCATTCGATGCCGGAACCAGATGGCGGTGA
GAAATAAAGGCAGGGAAACCGCCGTGCAGGGAAGACGGGGGCCATGGATGTGAAGGCACACCATTAATTTTCAGCCATAATAAAATC
TTAGCGAGTTTTGTCTGTGTCTGCACATTTGAACTTGCGGGTTATGATAATCTGTCAAGCCCCTTAGACCAATACCGTGAATCCAGG
CGACTTACAACAGCATTTACTCTCAAGTGCAAAAGTGCCAATGGGGCCCCAAGGTCCCCTGTGAATTTATGTGCTCTCTGCAAGAGG
ACAGTTACATGCTCCTCAACAGGGTGAATCCTGTCTCCTCCTCAGGAATCCCCCGGAGAGCATCAGGAATCATTGCAGAGGTGACTA
CGTCATGCAGACAAAGGGGCCCTGGCAGCAATGAAAGGGGCCATAGCTGCAATGATGAGGGTGTGAAAGAAAACTACAGACACACAG
GGTACGAGGGCTTTAAAAACAGACAGAGTCTAGGTTGCAAATGGTCAGAATAAGTCAAAAGGTTACCTAGCTGGGGAATGAGGCTAG
GGAAGAGGAAGGTCTTAGCATGACAGCCAGAGGGAAGTCAGATGATGGGGGCAGCTTTAGGGGGAAAGGTTGCTTGCAAGGCAAAAC
ATGTCCTTCCTGTTCCCTCTGGCCAAATGGCCAGTGGCTCCACTCACACATCTCATCAGGAGCACTGGAGCACCGGCTTGATCAGTG
TTGGCTGATGCTGCTGGCAGCTGTGGGCGTCTCTGCCAGCTGTGTGAACAGCTGTGCACCAACCCTGGGCTCTTCTATGCCCTGGCT
CATTTCCCATCCCTGCTTGGTTTAATACACTTAAAAACAGGATCCCACACTCCCTGGTTCTAGCTGTAAAATATGCTGTTCAGAGGA
AAAGAGCGTGTCTGCCTGGGCAGGACAGTGAGACAAATGCTAGATAGACGTGTAGGCTTTTCTTCTTTTGAAGAAAATCTCTGTCTG
AAGCAGACGGGTGTTGATTAGTGTGTTTAAAGATGGTGCAGTAAGGGCTAATAACAACACCAGATCGTTACTGAATGGCGGTTGTCA
ATTCTGACTGCTTCTCACAGGGCCCACCTCCGTTGGTGCAGCTTGTGTGCTGACCTTCAGCCGAAGCCCCAAATGGGACTTCTAGAA
ACCGTTTACAGTACCCTGCCTGGAGTTTGCCCAAGCATTCACAGATCCACTAGTTTGGCTCACCCGGAGTAAGAAATGGCCTTTCTT
CCTGGGCTGCTGGAGTCTATGAGATTCTGCCGAGGGAAGAGAAGCCTGGGACTCCTGGGTGGGCAGAGTCAGGATTTCTGAACTGTT
CACAAGGCCCAGTCAAGACCAAGGCCCAGGGCATGCAGCTGGCATGGAAATAGTGGCTCCATTCCCAGTGAAAGGTGTCAGAGCCTT
GACTTGTCAGGGCACAACATGAGAAGACCCTTTCAAAGACAGCCTGTTTCTTCCAAATGATCAGACATCCAAACATCAAGCATCTAG
TAGTAAACTCCAGGCTACTGTAACCGTTTCCTGCGCTGTAACATAACACAACCAGAAGGAGTGATAGGAGCCTCGGGGTTCGAGGGG
TTCTGCCTGCTGTTGGGAGCTCCCTGCATGCTGGTGATAGCAGGTGGGGCTGTACAGAGACATTCTGGTTTTCTGGGGTAGAGTCAC
ACAACATATTCCTGGCTGGAGAATTATGATGACTGGATCTAACTTTTTTTCTGTAATTTTCTAGGTAATATTTGTATGTGTGTTTGT
GTGTGTGTGCGTGGGTTGTATTTGTGGGTTATGAGGTTGCCTTTCTAAAACATGGTGGCCCATGCCCTACCCAACCACCCTTGCATT
TAGACTTCAGGGCTCTGTGCCTGGCTGAGCTTTTCCCTGCCCTGAGTGCTGCCTCACCTCCACCTGCCTTGCAGTCACCAGACAGGG
GACTCTACATCTTACCTGGACCTTTTCCTGCATGTTCTGAGGTTTCCCACCTCTCTGCTCTGGAGAGAGCTGCTGGGTGTTAAGGAG
GTGTCTCCTGCCACTCTGCGCAGGTTTCCTGAGTCACAGTCTTTGTCCCTGCTGACAAGCTCGATAAGTACACATGACATTTGTTTT
CCTCTGGCTCAGAGTCCAATGGATCTGGATCTAAATTTCAGCTCTGCCTTTTACTGGCAATATAACCCCAGGCTAGTGGTTTAGGCC
TTTCTACTTCAGTTCCCTCATCCATAAAGACGAGTGCTGCTCGCATCCTCCCTAACAGTGAAGTGATGTAGTGAGAGCTGTGGGTAT
GGGGGCCATATGGAGGCTCCCGTCTCATCATCCCCCTCATCGGGACCTGCTACTGGGACCCCATATCCTTTTGTGGTGTCACAGCTT
CAGGGTCTTCTCAGAGGACTTTCCCAGGTCCTGCTGTGGTTGGCGAGTGCTGCCAGCTGCAGGAACAGCTGTACCTCTGTCTTCTTT
CTTTTGCCTCCCCTGGGTTTTTTGCTGTCACGTTAGCACGCAAGTGCGCTTAAAAATAAGTGGCAAAAGCTGTGCTTGTTTGAAGTG
TAAGGTATGTCACTCTCAGAAAGCGCATTTGTGCTGTATAATAGATAAGAAAGAGCTATCTAGTTAAAGAACTTTTGGGGCTAGCTT
TTCTTCCAGGGCGTGGGAGGAGCATCCTCCATCCTGGTGTAGGCTGTTGGGAAGAATGAAAGTGCTCTTCAGAGGCCCTGCACAGGC
TCTCGGCCTGTGGCTTTCTCATTGATCTATTTCTCTCGAGTGAATGGGCTTAACAGAAATAGAGAGAGATTTTTTTCCCTAATGATC
AGGATAGAAAAGAATAAAAAGTGACTTTAGGGGTATCTTGGGACCTTAGCTGTCATTGTGTGCTGACACTTCATTTCGGAATGTAAA
GGGCCTGCTGCTGGTTTGTAGGGATAAATGGTCTTCTAGTCGGGGGCAGCCAGGAAAGAGGCAGAGCCCTTCACTTAATAACACACA
GCATGGAGGGGCCTCTCTTGGGTTGGGTGATGGTCTCTCTCTCTCCCTCTCTCTCTCTCTCTCTCTCTCTCTCTCCTTTTCTCCCTC
AGAGAAACAAGGCTGGAGCTTGGGCCAGCTGGAGCTCGGGAGCTCAGTATAGAGTCCTAATTAGTTGTTTGGACCAAATGTTTCCTA
GTATTCCTAGGAATCCAGGGAGGGGGTTTGCTCAGTGGGCCTTGGAGACCTGTCTTGGGTTGCTGCTCTGGGAACAGGTGCCCAGGC
AGGAGGCAGCTGTATGAGACTGCTAGAGTAGAGTGATGTTCAATCACGTGAGGGTCCTTATCCTACCAGGTGACAACCAGACTAGCT
CTGGAGAGTCTTACATTTGTCCAGGACTCTGCTGTCCTGCTCTCTGTCAAGCATATATGTAGACTGCTAGGAAATTCCTGTGGCTCA
GGGATGTGCCCAGGGTATAACAAAAGGCAGGGTGGGTGCATGCCAATCTACCTCCTCTCAGGACATGCACCCAGCTCCTGTAGTGAG
AGGGTAGCCCAGCAAGATGACTTCTGCTAGTGGGGTGGTGCAAGTTGGGAGGCCACATGCTCTGTCAATAAGGTTCCCTGACAGAAG
AGACAGTTGTAATCTTAGTTCCACAGAAGCTCTGAGATCTGCAGGGCTGCTCTCTCGCTACAAGCTCCTACAGTTGATTTGTGTGGC
ACTTATGAAGCCGAAAGATACCTGCATAAGCTTCGCCAATGGGCATGAGCTTTGGACTTGGGTATGTTCAGTCACCTCCATGAGTGA
GTCAGAGACAGAGCCCAAGGATACCACAGGTCTCAGCAGCCATCTCCAGACTGTCCTCTGATTGAGCTGATGAGGAAGCTGAGGCCT
GGAGCAGTGGGAGACCCAGTACAGTCCAGACAGCCTGGTATGAAGACTTGCCCTGTTTAGTGTCCAGCACTTTAGCTAAGCCTTAAG
CACTTGGCATGAGGGAGGCTCTGCTCCACACTCACCAGTTAAAACCAGTCATTTTCCCAATCCCAGGCACTGGAGATGCAGTAACAG
GCAGAAGAGATCTGACCCTGCCCTCACAGAGTTTCCACTCCAGAGGGAGGGGAAGGGGTGGGCCATAATGAGGGAAGTCTTAAAGAG
GCCAGGGTAGCCAGGCATGGTGGTGCACCCTGCAGTCTTTAGTACTAAGGAGGTGGAGGCAGGATCAGAAGTTCAAGGCCACCCTTC
GCTACACAGCAAGCTCAAGGTCATCCTGAGCTACCCGAGACCCTGGCTCCAAAGAAGCGTTTTCGGTAAATTCGGTGAAGGACAGTG
AAGAAACCAGGACAATGGTGGAAGATGATGGAAGGAGAGATGGATCAGGCTCGGGTTTCAAGGGAGACAGAGCTCTTTGATGGTAAC
TGGAATATCGCAAGATCTACTTCTGTTAGTATTGTTTCCTTTTCCTGTATACTGAAGTATTATCCGCAGGAAAGTGAGGCTTAGAGA
CTAATTAAGCTCTCCAGTCACACAGCTGGCGGACAGAAGGACCAGGATTTGAATTCTAGTTTGCCGGGCTCTTAGCCATCTTCCTTG
GCTGTTTCCAGTTCAGTTGACTGAGTTCTGAATCTTCATAGAAATGAGGTGACCGTTTATGTCACCGCAGGGCCAGGTCTGTGAGTC
TACTGCCACTGCTCTGACACCCCCTGTTCTAGGAAAGGGTGAGCTAAATATCCCAGTGGAGCACCTCCCTTCCTTCTGGAACTGGCC
TAATTAGGAATGAGGTGGCAGTGTCCCAGGGCCACCTTATTTAGGCCACAATCTCTCTACCCTAGGCTACTCATGAATCCGAGTGAG
CTCCCTGCAGGTTAGTACCCTGGCGCACCTCCTCCCAAATGTTTTTCTCATTTCCGGTTTCACGGCTGTTTTGAATCTACCAGCTTC
ATTTCATTCATCACCAAAGCAAGGCACGCGATAGAAAAGGCGCATCCCGTCCTCTGGGCGCCGCCTCATTTCCTGCCTGAGATTGAC
AAGTGCCACTGAAGCATTTTGCACACCTCAGAGAGGGTTTGCCATTCGTGTGGCCAGAGAAGTTGTCATGCAGACAGTTGTAGGGGC
TGGGTCTGCCCTGAGGCCCCAGGCTGCAGACACAACTCAGACTGTTTTATGGACAGAGTCGGTTCTTCAGTCATGACTTGTCTCCCT
AGCCACACATTTTTAGCCTGATCGTTGTGAGCTGTCACAGCCTGCACACCTCTCAGGTTTTAACCAGTTGAACGCTCTTATAAAGTT
CTGTGGACTGGTGCAAGAGCATCTCTGTGTTCATGTGCACGTGCCCACGGTGGGAATGTGTGGAGTGTCCTTTCAAACCTTCAGCGT
GTTTCTGCTGGGGACAGGAGATGTGTACTCCATGAACTATAACTCCAGGCTGAAGAGCCCTGGAGACCAGGGAAGGGAAGCGATTCA
GACCTGGAGCTGAGGCCAGGAAGAGGGGCCCCGGCTGTGCTCTGCGAGAACTGCGGACCTGGTGGGAAAGAAGGCTCTTTGAGCTCC
TTAGTTGCTGCTCATTGTAAACCTAGAGCATTTCCCAAGGGACACAGGGCATGTTTATTAAGTTTTCTTTCTTTATCCTTTTGACAG
TGGATTGAGCCTGGGACCTGGGCAGCTAGGCAAGCACTCTGCCAGTTACTTCTCCAGCGACTAAGCATTTTTTTTTAACCATCCCAC
TCATCGCCTCCCACTGGCTCTCACGGTGCTATGACAGTGGGGTCCTCTTCCTGGTGCTCCTGCTTCCTTCCCTTTCTGTGTCCTTTC
CTCTTTCTTCACAGCTGGCCAGGCATCAGACAGACTCACACATGCAACCTTGACCTTGACCTTGCCTGCAAGCTTCCTGAGGTTACT
TACTCATCGTCCTTTTGATACAGTTTAGACTCCATGAGACAGCTTGTCTTGGAGCTGGCCCCACCCTGCTCTGAAGCCATCTTTATG
CCACCTCCTTAAACACTCTCCAGTCACTGTGAGGTTCTGTCTAGCCTTGGTCCCATATCTCTGGACCTTTGCACATGTCATGTATCC
TTTGTTCCAAATGCCATCCCCTTGTGTGAAGTATCTTTTATCTCCCCAACTTCTCTCTTCCTTGTGTACATAGACATCTCCTATGTC
CTTGTCTGCCATGCAAAAACCTCTTCTTCCCTTCCCTGAGCTCTTCAAGGGCAGCCATGGCCATACCTCTGGAGCTCCAATCTCTAT
GGCATCAGACAGAGTAGGGGCTCAGGGAGCGCTTGGTAAATGAATGGATCCCCACAATGTGTACCCACTGTTAGGCAGCAGTTCAGT
CGACCTCCGACCTGAGCCACCACTCCTAAAAAAGCCTCTTTTTATCTGCAGGGACTCAAACCACCCATGCAGAGGTGGCTTTGGGAT
ATGACTTCATGTTCCCTCATATGTCCACAAAGCATATGTCTGAATGGGCGACTAGATTCTTTGCACAATATCAAGCCAGAGAAGGGA
CCCAGCTCCCCGTGATACAAAAGGGAACAAATTCCCAGAGGTTTGTTTGTTTTTGTTTTCTTGGATGATAAAAATGTGAGCGTCACA
AGGTCACAAGAACACAGAGACTGAGACTTTGGGACTTGGCCATTGTGGTCTATCTGTTCCCCTGGGCAAGATAGCAGGGTTGTTGTT
GTTGTTGTTGTTGTTGAGACATGGTCTCACTATATGGCCCTGGTCTGGAGTTTGCTATGTAAAACAGAATGGCCTCAAATTCACAAA
GATCCACTTGGCTTTGCCTCCTGAGAGCTGGGGGGGCGTCACCTCTTCCAGGGCTCCATCAGTGGCATTTCAGAGAATGTTTTGTGA
AAGTATGAAAGAGCTCACAGGATGCCTGCATGCTTCAGTAACTCCAGTGCAGACTCATAATGGACCCACCCAGTTGTTTGAAGTCCT
GGCATGAGTACTAACTATGAAGAAATGGCCTCCTTTCTTACATAGAGCCCTGTTCCCAGACTGGGTGAGAAATCGCTCTTCGCAGGT
CAGTGAACAATGAGCTCTTCTACATCTTCCAGGCAACTTGTCATAGAGTTCCTATGCCGCACACACAGCAGACATGACCAATCTATC
CCACCATCCTTCCTCAGTGAGCTTATGGGCCACTGCAGTGTGAGATGCCCAGCCTGGAGTGCCTGCAAACCATTTTTGGTCTCCACT
CCTTGAGGCTCTGTCCCAGTCATGACTTTGGTCTTTGGCGTTTCTTCACTTCCATCCCCTAACCTGGCTGCTCTGGCCATTCTTGGA
GCTATTTTACTACAGTCATTTTGTGTTGTGTTTCTGCCACATCCACAGCAGTATGGCAGTAGCCCCCAGCATCTTAAGTAAGTTACA
GGATATACTATTTTTCCATTATTAAAATTATTAATGATTCATTGATATTTTGAAGAGTTCATTAATCACGCTCATTCATGGTAACTA
TTAGCATGGTTTATTACTTTCAGCTGGGGTCTGTCTATCTTCCAGCCTGGTGATCTTGCCCTTGGGCTGCACTGGAGTCTTCTGACC
TTCCTGGTTGCTCCCCTGTGCCTCACCACTTCATCTCTTCTGTTTGCTATAAGGTGCAGTGAGGGCAGCTGGGGTAAACTTCTGTCT
GCCTCTTACAGGCATTTGGGCACCTGCCTGGCCAAAAAGCAAGGTGGGTGCTTCCTCTCTGGCTTTCTCCCGGGACGGAGGGGAGGA
GGCCACCAGCAGTTGTTCACCGCCCTGCTGCTTGAAGACTCTGACTTAGCATTCCTGGTTTGCATGTCTGACACCTCACTTGGCACA
TGATTTATTACAGTTTTAAATAGCCCTTTCTTTCTGGATTGAACTGTCCATATCCATTTGGAGTGGAAAGACCTGGCTTGTGGTGTT
GGTAGTGCAAAATGCTTTTCTGAGTGCGATTGTTTGGGGTTTTTTAGTGTAGCTGATGCTCTCAGACCAGAGTGCTGCTCCCTGTGT
CTGGCTGGAATCCAGGAGACAGAAGCAGACATTTGCCTGCCACCTGGATGAAGGAGGACATGGATGCAGAGAGCCTTTTGTTTCTTT
TCGTCTACATTTCAGAGACAGAGCATCCCAGGTGAGGGAAGCAAGTCTTATGAACAGCCTGGAGGGGGGTTCAGAATCCATCCGAGC
CATTCCCCTGCCTTTGTGGTGGGCTTTACCTCTAGGTCTAGGCTTTGTTAGGAAAACACTTCCCAGGGAGAAACTTTCCTTGGTTTC
TCTCTCTTTCTCTCCCTTCCTTCCTTGGGGTTTGGCAAGTCCTTCTAGGCAAATCCTTTTTGCCGTAGTTAGAGCACCTCAGGGATA
GTTAAACTGGAGTACAGGATGCTTACAACAAAACATGCAAATTCCCCTTCCCATTTCAGAGACATATGTCCCCTAGATGTCACCAGA
ATTAGCTCATCAGATTGTAATCACCATCCTGGGATTATGATGCTGTGGAAAGCAGCAGAAATCCTTTTGAAACCCTTTCTCCTCCAG
GGGGAGGGGGAGCCCTACCAGCTTCCTGCAGAACATTCTGGTTCCTTTTGTTATCCTGCATTTCTCGACTGAGATCCACTAACAGCC
CACATCCTCTGTGCTGCTGGTTGAAAGGCCATTTAGTGTGGTCCCAGTGAAAAGAGAGAATTTGGCATGGCTGTAAGAGCCTAGCAG
AGAGAGGAGCTGAGGGGACAAGCCACCTGGGAGCTGCATGAGTCTCAAAGGGAGGCCCACCAGCCACATCCAGACCCTTCATCCCTC
ATCAAGATGCCACAGTGAGGGCCACCAGCCCATTTCCACCTTAATTCCATTGGAAGCTATACTTCTCCCTGGAGATTCTGACCCCTC
CATGGCCATGAAGCAGAAACCACCCATCATGGTGGCTTGGGTTGTTTGTTTCTTCCAAGGGTTTCCTGGCTAGTTAGAAGGGTGATT
TTTTTCTTCAATATGGGATGAAAGAAAAAGACAAGATTTCTGAGGGTTGGAATCATCCAAGGAAGGAGAGTTAGTTCTCGAGGTAGG
TGTGTCTTCCCCTTCCAAATTCACACAAACAATTAAGAGGTTGCAAGAGTTCTCTGGAAAACTACAACCTACTCATTTGGCCACAAG
AGAACACAATAGTTAGTGTCCACATGCTTTAAGTACGATGTCTTTCTTGTTGGAGAGAGCTCTAGGTTATCAGTAAAATACACAGCT
CCACTTGAAAAGCTTTTTAAAAAAAGAAACCCAGCTGAGAGCCCCTTCCTCTTCCCGCTCACGAGCCCCCACTCCTGCCTCCTCCTC
CCCTTTTTCCCTGTTCTGCCCTGCTACATTGCTGCAACCAAACTTGGCACGGTTACTCAATCGCTAGTCTGAAGGGCTTTTAAAACA
CGTTCACAGACGTGCTCTTGATCCATTCAGTTGTCCAGGGCAGACCCTCGTGGAGCCCTACTATGTACCAGCTATAGAACAGACAGA
ACGACGGACTGTGGCTCCTGCCCTGCAAAAACTGTTAGCACCGAGCAGGAAGTCTTTCTTTTTCAAATTATGGAAAAATTCACATAT
TATAAAATTTACCATTTTGGTTTTTTCCTGAGACAAGGTTTCTTGTATTCCAGGCTGGTATCAAACTCTGTATGTTGCCTGGGAGGG
AAAGGGGGAAGTGGTGTAATTATAAAAGAGCCATCTTGGTGGGTTTAGTCAATCCCTTCCCCCCACCCCCCAACCCTTAATCCCTAG
ATTAAGTGTAGTTAAATCAAGGACCCAGAGATGGGAGCCAGTCCTGAGTTATCTGAGGGCTCTGCTGACCACAAAGTTTCTTGTAAA
CAGGGAGGCGGAGAGGCCTGACCCCAGAGGAAGATGTGAAGCCAGAGTCACAAGAGGTCCAAAAGACTGGGAGGGAAGGAGAGGGGG
CTGGAGAGGGCCAGGAAGTACAATTCCTCAGCCACCCTACACGACACCTGGACTTAGTGCAGAGTCATCATCTGATCTCCCGAACTA
TGAGACCACACATCTGTGATGCTGTGGTGTGTGGAGGCCGCAATAGGAATCTGACACATGGACATGCTGGAAAGGCCGGAGAGGCTG
GAGAGGCCGGAGAGGGGTCGATCCAGGCTAAGCTGCCGAGCAGCTGTGTGATTTAGTCTCCTCGTGCCTCAGCTTCCTCCTCTTTAA
AAATAACTGGGCTCCCACAGCGGTTTGTTAAGGGTATGGAGTTAATATTTGTAAGTCACCGAATGCCTGGAACCAACAAGTGTGCAT
TTGTCAGATAAATAAAGCCCAAAGCGCCCAGCCAGGTCTGCCTCGTAGGTCTCAGTGGGATGATCTCATAGCTGAGTCAGTGACTTG
AGTCTGGTCAACTGGGAGCTGGAGCCGGCCAGGGAGGATGGCAGCTTTTGGAGCCTGCTGCTACATCATTAAATCGGAGCACAGATG
CTTGAAGGGATGGCCCCTCTATTCCCCGTCACACCACACCAGGCCCAAGTACATCTGCGAGGCTGCATCTCAATGCTGACCAGTCTG
CTTCCCTTTGGGAACCACAGAACGAGACCAGTGGATTCTGGGAAAGTGCTTCAAACAGCCAGCACTGGCCCCTGGTTCCTGTGCTTC
TCCAAAGACCAGGGATCTCTGAGCCGGTCTGCAAGCCCTGGGTTGGTACAGAGAGCAAAAGAAGGCAGGACAGGCCAAGAGAAGGCA
TCATCACCATATGCCTGACCTTGGAAGGATCTAGAAGATATGGTTGTGGCTTCTACTGCTGCTATAATCCACCTCTTAGATGGTCAG
AGGCAGTTGTGACAGATCCATTTCTGACATTTGCAGGGCTTACAGGTCAGCCTCACATGGCAGCACCTAACATCACTTCAGTCGTCA
CACCCAAGCTCCGTCAGGCTCCCCACCCCCAGGTATGGGGAGCATCTCAGTACCATGTGGCCCCGGGGGGAAGGCTTGGCTCTTTGG
ATAGGAAATTCTGGGGTAACTGGATATCTGGACATGAGCTTTTGAGGCGGCCTGGCCCATGGGTTTCCAGCTCTGTGAGATGGGGCG
GGGCAGAACCCTCATGTGGCTTATAGATCAGGAGCCCTGGCATGTGGGTCCAGGGCAGTGGTGGTTATCAGTGCAGTGGTCAGGGCG
CTGGATGGTAACTAGCAGCACTCTCTCCTCTGCCCTGTCACCATCGCAGCCACCCCAGTCTGAGCCTCACTTCCAGCACAGGCTTCT
GTGAGACAGCTCTAAACCTGTCTCAGCGTGTGTGTGTGTGTGGGGGGGGGGGTGTCACTTGAACCCTACAAGAGGCCCAGGGACTCT
GTCTCCTTGGTGGTTTTGCCTCTGTGGCTTTCTTCAGACCCTCCCCTGGAGGGAAGGGGCAGTGGTAGGTGGCTGATGAGTCCAGCG
GTGAGGAAAGCACCTCCTTTGGAATAGGGAGCCAGCTTCCATGGTACTGGGCAGGCTGAACACGCTGTGGTAGCTTTCAGGACCTGT
AATGGGGTCGGCCTGAGAACAAGAACCTTGTTTAATTCTTATGCATAAGAAATCTTTTTCACAGGGTGGCTTAGTCCGGAGGAAGTC
ATGGAGCTTGCCTTGGAGCAGAACACTTTGGGGACCCCCAGGAGAGGCATTGTCCAGCAGTGTGGGGGGAGGCTGGAAGCTCTGCAG
AGAAAATAGCAGGGCGGGGGCACACTCAGAGGATCAATTTCCCTGCAAGAATGGCCAAGCTTAAAAATATCTTCAGTAGGCTCCAGG
AGTGGCTTTTCCCGGGAGAGTTCTACTAAAAAAACACCCCATTTTATTAACTTTTTAAAATTACAGAAACAATATTGGCTTGTTTTA
GTGCAGAGGTGTGTTCTAACTATTAAAGGTTAAAAAAAAAAACAATAGTTCCCTCGATTCCCGTTCTTACCTCCCCAGAGATGTAAG
ATGTCAACTATGTGATATTAGCCAGAGGTGGTCTGCGCACCTGTGTGCATATCTGTTCACACTTACCAGAAAACCAGGTCACCGTGT
ACACATACGGAGAGATGCTTGTCGGCATGCTGCCGATATTAAAAGGGGGACTCACACTATGCCTCACTGCATGGTCATTTTCACTGT
TTGCTTAGCAATACACTGTGAACCTTTGCTAAGTTAATACATATAGACCGGCTTCATTTTAACCAGCTGCATAATGTTCCATAGTGT
GAGCACCATGGTTAGTTAACTGGCTCCCTACGGATGTGTATTCGGGTCATTTTCCAGGTTAAAAAAAAGATGTTAAACAAGCAGCTC
TTTAGAGAATGCTATTTGATTTCTTTATTTTGGGCACGTCTTTATTCTCTTGCTTTGGGAGAGTTAGAGAGCTGGTGGTCTTGGGAG
TTCTTTTCTTTTCTTTTCAGTCACATTGACATAGGTGAGAACTTACTGCATCTAGATCCATCTGGAAAGCCCATACTTTCCTGAGCT
TGTGCTTGGCGCCCTGGCCACGTGGAAACCAGTCCTCAAGAGCACCGTGTCAGCCAGGGGCCGACAGGCAGCAGCAGGTCAGGTATC
CTTAGTTCAAGCAGGAGCTGCTCGCTTGGGAGCAAGCTGGAGAGCGGGGTTGTGGCTGGAAACAGGTGTTTATTAATGAGGGTGTGG
ACTCCATCTCTCTAACCTGAGGTCTTGGGAGACCCAGCTTAGCTGTGGGCTAGAGACCCAACAATGCTCTTCTCGTGGGGCCATTGC
GAGGAAAGAATGAGCGACTGCACCCCAAACTCTGTAGGACAAGAATAGACTCAAGCGATCTAGAGTACAACGGAGTGACCAGAGTAC
ACAATAATCTGTTGTGTATCCTATGAAGCTGTAGACATAGGGGTGTGTAAGAAATGGCAGAGGGACAGGGGAGATGGCTCAGCAGTA
AGACTACTGTTACTTTTCCAAGGGTCCTGAGCTTTGAATCCCAGCATCCACGTGGCAGCTCACAACTGTTTATAACTGCAGTCCCAT
GGAATCCAATGCTCTCTTCTGGACTGCAATGCAAGCAGTCAAAACGCCCATATATAAATAAATAAATCTTAAAGAATTGGTAGAGAT
AAAAAGCTAAGCCCCTCTATTGGCTCAACACACATGTATTAAAGTATCACATACTGTGCCCCATAAACATGCAGTTACTACATGTTA
ACCAGAAAGAGACAAAGAACCATCACTCCATCAGAACTGTGCAAATGGAGTAAACAAGTGGCCAGGTGCTCCCTGGGAGAGCAGTGA
GTGCTCCCTGAGGGCTAGTACTTCAGAGAGTCCTTTTTCATTTGTTTACTTTTAATTTTTAAGAATTACATATAAATGTGTTTATTT
AGCTACTCATGTGTGTGTACATATGGGCATGTACATACCACAGTGAATGTATAGAAATCATAGAGCAACTTCCAGCTGCTGGCTCTC
CCCTTCCATCCCTGTGGGTTCTGGGGGTTGTATTTAGGTCATCCATCAGACAGGCATCTTCACCCACTGAGCTGTCTCACTGCTTCT
TGCTGTCTGTCTATCTGTCTATCTGTCTATCTATCTATCTATCTATCTATCTATCTATCTATCCATCTATCTATCAATCGCAGGGTC
TCACGTGGCCTATGCTGACCCTGTACTGCCTCGTGCCTTTATTTAATTTGGGCAGGCTCTCGGGTAGTTCAAGCTGGCCTTGGACTC
CCAGGATAGCTGAGGATGACCTTGAACTCTCGATCTTCTTGTGTCCACCTCCCAAGGGCTGAAATCATAGGTGTTGCTCCTACATCT
GGTTTCTTTTTGTTTTTTTCTGAGACAGGATCCTCTCTGTCATTCAGGATGGCCTTGAAGTAGCTGCCCGGTGCTGGCATTGCTAGC
CAAGGCCTCCGCAGCTGGTCCTGGACAGTGAGTTGAAGAGCCCTTTAGTCGGCCCATGACACACCAGCTCTCAGATCCCAAGGGTGC
TCAGTGATTCCAAACCTCTTGACCCGAGTTGTTGAAAACACCCTTTAAGCCATGGCTGAAGAATCTCCAAACAGTAAATAGGATGGG
TTACTGGCCTGGTGGCCGGTGCTCAAATCGTTAACCCACTCCCCATCCTAATCTTCCCAAAGATACTTTCCTGTGTTAAGCTAAAAC
GAAAACTTAAGAAACTTCCTGTTTGGAAAACCATTCTACTTACCCCAGGCATTACAGGTGCCAGAAGCTGCCACTATCAAGGTAGAA
AGAATTGGAAACACAACACCAGCTTGGTCCATGATAGGCAAACACCAGATCACCAACCCCCCAGCATAGCCTGGGCTGGGCTGCAGT
GGTCAAGAACATAGACGTACAACAGAGTAGATTTTAAAAGAGTCAAAACCTGCCCTTCCTTAACTATATGACCATTATTAAGTTCCC
ACAATTTTCTGAGCCTCAATTTTCCATCTGTACAATGGGACTAACCACACTTTTGGAACCCTGTTCAGGACTGAATGAGATGGTGCT
GTAATGCGCAGTTTATAGGGAATGGCATTGAGAGAGCACTGTTGCTATTGAAGGGGGTAGCTTGGAATTGGGGGGTGGCTGGCACTG
CTAACTTCTGGCCATTCTTTCCCAGCCTCTGTGAGCTGGGGACCAGTACTGGGAAGACCCTGGGAAAGGTGTGAAGATGAGTGGAAC
TGGAGAGATGGATCAGTCCTTCAGGGTGCTCTCCCGGGAGAAGCAGGAGAGTGGCACAGTAGTTGCTAACACCCCACGGGACCCAGC
TTGCCATCTGACTGTGGTGCTGGGTGCACTGTGGCTGATGACGAGGAGGGAGGTTCAGCTGAGAGCAGGTGGGAAGAGCTCTTTTTT
TCCAAGTGAGCTAAGTGGTTGAACTTGACCCAGAACCCCCTGGCGGACAACTGGCAGGGTCCAGTGAGAGAAGTTATGTGGCTTGGT
TCTGTTTGGGAACACTTGGCACTGGAAGGTGGCTGGAATGGGGCACTTGCTAAATCTAGGGCGTGGCCACTGTAACCAAGTCTGGTT
GAGTCACTGTGGCCTTACTGCATTGAAAAGAACTTGACAAACTCCAAACCTACCTGAGGAAGAAGGCCTAGTGTCTGCAGAAGTCTC
GGGGAGGGAGGCTTATTGGAAACTTCCAGAGCCAGTCTCGTGTTTCTGCGGAGTTCACGCCTATCAGGCACCAGGTCGTCTCCATTC
CCTCCCTTCTCTCCATAGGCTGAGCTGTCCTGGATGCCGGGTAGGACGCCATTGTCTGCTCCCAGACTTCTCTATTTCCTTCTCCGG
TCTATTTTCACCGCAGCAGGCATCACGTAGGGAATCCCATGGTGCATGTTTACTTGTTTCCCCACAGCGCATGCTCCGTGGCCCAGG
CTCCACTTGTTTTCTGGTTCTTGGAGTTTAGTCCACAGTCAGAACCCAACATGGTGGATAGATGGGTGGACCCATGAGCCCTTAGAG
TAAAGCTCCATGCCTGTGCATCCAGCAACATAACATTTTCTTTTTGTAATTTTTAATGGATTTGGCTGTTTTGCCAGCATGTACATC
CGTGCTGGAAGTGGGTGTAATGCCCATGAAGGCCAGAAGAGGGCACTGAATCACTTGGAACTGAAGCTTCAGAAGGCTGTGGGCCAC
TACAGGGGCGCTGGGAATCAATTCTGAGTCTCGTATAAGCACAGCATCTCTGAGCATCTCTCAGGCCGTGCATTCAGGAGCGTTTGA
ACGTGACCTCTCGTTCTAACATCAAACCTTGTCTTTAACCACAGCCAGGGGAAATCAACAGTTTCCCTGCTAAACCGGTGGGGCACG
GAGGTCGTACAGAGATCTGTGTAGGTGGAGGGGAGTGTGTTGGCACTGAACAATGTTCTCCACCCTTTAGCCTAGAAAGCTGAGTTT
CGTTCTCATATTGACCTCACATTAAAAAAAACAATTCATTAGTAGGAAATGGAACGCTGCAAACTGGATCCAACCCTTGTGTTCCAG
ATGAGGAGTCTGAGGGCCAGGGAACTGAGAAGCACTTGCCAGCAACAGAGCGCTCTGTCCCCATTGGCCCTGCTCAGGGATAGACTC
TTGCTGTGTGACTAAAACCTTCTCACATGCTGTAGCTTGACTTTCATGCTCCAAGAACTGGGGATGGTCACAATGATTTATGGTCAT
CACTGGCTTCCATTAGCCCCATGTGAACTTCAGACGCCAAAGTCCCAGAGGGTTCTCATGTCTACAGCAATAGACTAGAGAGTTCTC
AGGGGCACTTAGGAGGACCTTGGTCTGGCTGGCCCAAAGCCGCCATGGGACATCACACATGGCCCAGTGCCACCAGCCACAGGAAGT
TAGTTTCAGGTGGGGAGGATTTCTTCTTAGAAAGATGCAGGGCAGGAGAGCAGCACTCACCGTGCTCCGGCCTTGCTGTTGCCGCAT
GATGGCTTTTCTGACAAGGCCAAACAACGTGGGTTTTTGGTTTTGTTTTGTTTTGTGTTTTGAAATTTTGTTTGTTTGTTTTTTTGG
GGGGGGGGGTCTTGGGTTTTGTTTTTGTTTTTTGTTTTTTTTTTTTGCTTTTTGCTTTTTAAAAAAATTTAGTTATTTTAATTTCAC
GTGTATTGGTGTTTTGTCTGCATGTTTTTCTGTGTGAGGATGTTGGATTCCTTGGAGCTTTGCCATCTGAGTGCTGGTAATTGGACC
TGGGGCCTCTGGAAGAACAGCCAGCACTCTTAACCTCTGAGCCATTTCCCTAGCCCAACTTTTGCTTTTCAATTTTAATTTTTCTTC
GTCCCTTTCCTCATTTAGCTCCATGTGGCTTCTGCTTACTGGAAATTTTATGTTCATGGCTGCTGTAGACCTGAAGTTCATCTCATC
ACCGACAATCAGATTTTGTCCTCAGGGAAACCCAATTAGGGATCACAGGACTTTTTGATGTTGTTTCCATGGTAACCCCATCGTGAT
GGTGCACATACATAATATATAGTCTAAGGAGTTTATGTTTATTTATCTAAAAGCTATATATTAATAACACATTTTAAGAAAATTTTG
ATTTTTAAAATCTGAAATGTTATCTTCTGCATTAAAACATCATTCATTTATTTTTAACTTTGTGTGCATTGGTGTTTTGCCTGAATG
TATGTCAGTGTGAGGATTTTGGATCCCCTGCAACTAGAGTTACAGACAGTGGTGAACTGTCAGGTGGGTGTCGGAAATTGAACCCAG
GTCCTCTGGAAGAACAGTCAATACTTTTTTTTTTTAACTGCTGAGCCATATCTATAGCCCCTTCTTCTGCTTTTTTTTTTTTTTAAA
TGATGGAAAAATCCCCTGAGGGTTTTTCTCCCACAGAGGGAACATAAAAGTTCCCCTCCCCCGTCTCTTCATTTTTCTCTCTTTAAT
TTAGAAAATTTTATAGTTGAGTAAGAATGAACGCTTTTCATTTGGCTGGAATCTGTCTACACATTGTCTCCCTTGATGGCTGAGAAC
GTTTTATGAGACAATTTTGCTCAGATAAAGAGCAGAAGGGCCCAAGTCTTTCAAATGGAAGGGAACTACTGATCTAATGAGACAATT
CAGACACACTGCGACAGACAGGACAGTTAAGGCCTCGCCTGCTTCCTTGCCAGGGCAGCCACTCCCCAGCCTGCAGCTGTCCCACAG
GAGGCTCCAGGGTGTCTGGGTGAACTGACCTGTTCTGAGGTGACCAGATGGCCCTTCAGAGCTGGCTGTCACGAGCCAATGATGATA
GAGCTTCTCTGAGAGACTCTGTGTGGAGGAAGTAAGTCGCACCCTAGTGAGCTGGAGGGGCCAGGGGCTCCTCTGAGCTTAGCCTGT
GGAGAGCAGGCATCTGGGGCAAGCTCAACGCTCGCTCGCACACCGTGTGCTTTCTGAGATCATTTTCTTTGGTACCATCAGTGGCTA
CTCAGAGTTCGGATACTCGCAATGGATACCTTCGCCCCTGCCAGCTAATCCCGGAACTCCCTAGATCTGCTAAGGAGACGAGCTGAT
CTGGCACTCCATAAATTCTGCTCACCAGTGAACTTCCTTAAACACTTCCAGAAAATGCTTTAAAATAACGGAACAGGTCAGAAAAAT
AACTCTTTAACACTTTATTTCTTGGTGTCTGAAATGAAGTTTCCGTAAGGTGGCAGAAGGAGGGATGGCTATGATCTTGGCCCGCAC
TCCCCTGGACCGGCCAGCCCTATAACTACAGGGGTTAGAGAGTGTCAATAGGTATTGTGGGCTCTTTCTAGAGCTCACTGCAGGAGT
ACTGAAAAGGGAAGTGTCAGGTGTGGAAGGCCAATCACCTACCTGTCCTAATATCTATAGGCACACATCAGTATAAGTATATGTTGA
TGTGGTATTGTATAGCAATGCCCCTGAAAGACAATAGCTTAAGAGTACATCTGTGTAACTGGGCTCAGTGTGGTGGTCCTGTGCAGA
GCTTGGTCATGTGCTGGTCTAGGAAGGCCTTGCTGTCCCCCCTGACAAAGTTAGGTGAAAGAGAAAACGAGGCCATATATCTCCAGC
AAGCCCCATAGAACTGATCTTCTAAATGTCAGATGCATCGAATTTTATAACATCCCATTTCCCAAAGAAAGGCACTCGGTCAGGTTA
AACTCAAGATGTTGAACTATAGGCTATGCCTCTTGCAGGACGAAGAGGCAATGCCATACTCACAGTAGCAAGTGGCTCTGTGGACAT
TTTCTGTAATCGACATCTGGTACTTGTCTCCAAGCAGTGCTGAAATCCCAAGTTCTCCTTTGAGGGTCCTCATGTGCCTCTCTGATG
CTAGAGCCACTTGAGACTGCAAACTGGCACCCGGGACATGTGTTATGTCCCTACAATGGCCTAAGCTGGTTGTTTCTCGCTGGTTGT
TTCACTGAATTGAGCTGAGTTGAACTGAATTACTTCTGGCTTGACTCTAATTACTGCACATTAGGAATCACTCTGTTGTGTCAACCA
GCCTCCAGAAGCTCTTGGGATTGCAGTAACTCCCGCTCACAGGAATAAGAGAGGTAGTAGAAGGCTCCAGCCCCGAGCAACCTCCCA
CCATCCGAGGAAGGAGTAGAGTTTTGGGAAGTCCACTGGCTGTGGTGCCACATGTCCCTTCTGCAGACTGACCTCCCCCACCCCTGG
AATGGGGAAAATAGCTTTTCCAGTGTGAGGCAGCACAATTCAGCAATTTAGAGACAGAGTTTCTTTTCCTACCCAAGGAGTCAGTGG
ACGGAGCAGGAATGGAGGCCAAGCCCAGGCAGGGCCAGGAGAGAACTTCCTTGTCTCTGACATCTCTCCCTTCCAGGGCTGTCTGCA
GCTCTGCTCTTGCCGCCTGTCAGCATCACCCAGGGCCTTTCCAAAACCCACCAGAGGCAGTCAGGCTGAGCCATTGAGATCAAGCCC
CGGGGGTGACTGGTTCCTGGTGGAGACAAGGTGATTGGTTCCTTGCCCATTCCCCCCAGATCACTCTGATCCAGCACCACTCGAGAT
TCTGACTCCAGAACCAGCAAGATGTTCAATAGGCAAAGGTGCTCAGCCGCAAGCCTGATGACCGTGCATTCAATCCCCAGAACTCAT
GGTGGGATGGAAGAACCAGGTCTAGCAAATCGTCCTCTGGCTCTCCACACAGGCACGTCTGCATCTCCACAAACGAACGAACAAACA
TAATAAATATAATAATAGACGTCACTTTGAGAAATAATACTAGCTGCCTGTTCCAGGTTAGCTCTGGAACTTTCTGATGTTGGAGAT
CACCTGGGGCAGTTTGTTGCAGAGTGGGCCATCCTCTGGGAAGGGGGCAGCATCCTCCAAGGTGCAAGATGTATTGCCAGGCCCTTT
GGCAGCCATAGGCCTAGGGATGAATTTCCTGCAGGAGTTGTCACTCATTCAACAGAAGCTGACTTAGTGCCACAGTGTGCCAGCAAT
GCTGTAAGCCAAGCGAGCCAGTATGGGTTCTGCTCTAAGGAGCTCACAGAACACACGCTAGGTAGGAGGCGCCGGCTGAAAAGTGCC
ACCGAATTCTGGCTCCTTAGAGCAGGATGCATGTGCCAGTTAGGTTCCCCCATCCCCAGCTTGACATAAGCTAGAGTCAACTGGAAA
AAGGGTGCCTCAGTTTTAGAAATGCCTCCATTCCTTGATTAATGACTGACGTGACAGAGTGGGTGAGGCCACCCCTGGGCCAGTCGT
CCTAACAAAAATCAGGCTGAGGGAGCCTGGGAGGAAGCCAGTAAGCAGCATTCCTTCCTCCATGGCCTCTGCGTCAGCTTCTGCTTC
GGCTCCTCCTAATGTAATGATAGTCGGTAATCTGTAAGCCAAATAAGCCCTTTTCTCTCCAGGCTGCTCCTGGTCCTGGCATTTTAT
CACAGAGACCGAAACCTAAGACAGTGCACACTGGGGACCATCATGAAATCTTTATCTTCAGAGGAGGAAACTGCCCAAGAGAGGGAA
GATTAGCTGCCCAAAGTCACATAAGTGGTAGCATTTAATAAACATCTACTACTTGCTAAGATTGAGCTTAAGCATTGGCAATAGTAG
AGATCAAAGAGAAGCTCCCACTGAAATCACTGGGTCCCAGCCCTGGTTATCCTTTGGTATCCAGGAGCTGTAAGAACAATCCAAGGC
TGGGCAGTGGTGGCGCACACCTTTAATCCCAGCACTTGGGAGGCAGAGGCAGGCGGATTTCTGAGTTCGAGGCCAGCCTGGTCTACA
GAGTGAGTTCCAGGACAGCCAGGGCTACAGAGAGAAAACCTGTCTCAGAGCAAAAAGACAGAGAGAGAGAGAGAGAGAGAGAGAGAG
AGAGAGAGAGAGAAGGAGGAGGAGGAGGAGGAGGAGGAGGAGAAGAAGAAGAAGAAGAAGAAGAAGAAGAGGAAGAGGAAGAGGAAG
AGGAAGAGGAAGAGGAAGAAGAAGAAGAAAGAAAAAGAGAATAACTCCAGGACCCAGGATGCTGAGGCAGGGAGATTGTAAGTTCAG
AGGCAACCTGGGCTAGATAACAGATGCTATATCAGAAAATAAGTACGAACACTCCAATGTCAGCCCCTCCCTGACCCACCTCCCACA
GGTTCTTGGTCTGAGAATGGACCTTGGCAGAGGGATGCTTTTCTTTCCCTTCCCCCTTCCTTCTTTCCCCTCCCTCCCCTTTCTTCT
GCTAAGCCCCTGTTGTGCAAGGCAAAGGGATTTTAACAGGCCTCCAAAGTGGCTCTGATGTGCAGCCACTTTGACAGCCATTGGTCT
AAGTGACAAGACGGACAGAAATGCAGAAAAGGCCAATAAATAACTGAAACTTGTGATTGGCAAGGAATGAGCAGGAGGCAGAGGAGC
TGCAGGGTGATCTGCAGGAAGCGAGGTCACACCACAAAGCTGTGATTCCAGGGGTAGAAGGCACAACGCTGTCCTGTATAGGAGAGC
CGTGGATGTTCTGGGAGATGCCCGGCTCTACAGAGCCATCAAACCCTGTCATGATGCCTGTTTAGTAGGTACTCTCTGTCTGCAGCC
TGAAATAGTTCTCCTTGTTCCTCTACTCCAAACAACCTCCTCCCCCAAGTACACACTGCTTATAAATGCTCTAGCAGCAATCTCTGT
GATCCACCTAGAGACTAGTCAGTGGAGTGCCCAGGAGCAGGTGTCCCATAATGGAGACAGTCGAGCAGACTCTGAGGTCTAACACCT
GATGTGGGAGGTTCCAGCCAGCTCACCAGCTTCTGACTTACTACGCCCATCTTCCTTGCATCAAGAAACTGAGAGGCCAGTGGGGAC
AGCACCCCAGGCCAGTTCTTTCCTGTTGAGCTGTGTAGCTCAGTGGGGAGACCCCATCCTTCCGTTTAGATGATGTATCAGCTCAGT
CTCCACCAAGCAATCAAAGGAATCCGTTACTCATTGCTTCAGTTCCTACTGCATGAACCCAGTTACTATAGCAACTGAATGTGCTTC
TTTATTACTGGATGGAAGGCTTTAATTTTGTATTTAAAGAGTTTGGCCAGAGAGTCATGTTGCCATGACTCGGGCTGTTGGATAGCA
GTTAGGAGCAGACAGACACCTTGGAAATGGCCATAGATCTGTCTTTCTAATTGACAGATCGGGGCCTGGGAAAACCTAGGCTGCACT
GCCCTGAGAAGTCTCCCCTGGTGCTGCGCTGGTACCTTCCCCTGTCCACGGATGGTTCCTGATGAGCAGAGGCCCCTGAGCCAGGCA
TTCCCATGGTGGCCTCTGCTTTCCCTACTGAGCTTCACCCAGGGGCCTTGCTTGGATGTTTTTGCTTTACCCTCTCCCTACAGGCGG
CAGGCCTCTAATGTGTTTGCTGAATACTGGTTTATGACTTCATTGAGTTTCACATTGGTTATGGCCCTGTATGGTCTCAGGACAGGA
AGAGGGGAAGCTGAGGCTCAGTGAGGTTAGAGTCTGGCCCACCCCACAGCCTCCCCTTTTGCCTCTCAGTGGGCTGCTAGGATGTCT
GGAACTTGATCCCACAGCACACAGATGTCACAGGCAGACACTAAAAGCAAGAAAGGCCATCTCCACACAGCCCTCCACGGCACACAT
GTGCTGTCTACCTCATGTGACTCACCTGTTTGCCTTCTATTTGTATCTCGTGTGCCAGCGACTCTTTGACTCTCAGGTTTTAGAGTC
TTCTCCTTGGATGAAGGGGCTCATGGGAAGGCAGTGGAAAGGGGTTAGGGGGATGCTCAGTGACTGCCTTCGAAGCAGAAGGGTAGG
GTCCAGACCTTGGAGGACACAGAGGTAGTCCACTGCAGGCAGAAGTGGGCGGAGCAGGAGCCTTAGTTCATGTAATCTGTGACCAGC
CCTCAACTCCCCACACAGCTGTCTTTTCTCCTCTGTGAATGCTGGATTTGTGGTTAAGCTCATCTTTCCCCAGCTTCTACAACATCA
TCGGCTTCTTTCTCTCCCATCCTGACTTTTCACTGGCCTTTCAGAACTGGTCCCAGGCAAGATTCAGAGGTCACTCTCCCTTCATGA
TGTGCAAGTGGGAATGCCTGTCCTCCAAGGCTCACTGCCCCCAAACACCAACCAAGTTAGGCCATAAGACCTCCACCCTACGTCTTC
TCTAAAGGCTAGAAGAAGCCATGTTGGAGGGCAGTAGTTCCCTGGGGACCTGGGCAGAACACATGGGGTGCATGGCAGCAGATGGCA
TCCAGGGAGATGGATGTATACAAGCAGCCTCTGTAGAGGGTATAGCGAACACCACCATTGTCCTGGCTATAGCTCCAGCCTTATGCA
GGCCCCAGTTGCTTGGGACAGAGTTGCTGTCAACAGAGAAGACCAAGCTTGTTGAAAGGCCAAAGTGGAGTTCCTGAACAGAACACA
GAGGAATCCCATAGTAGGGCTTCCACAGAGACAAAGCCTGGGCCTCCCTCAAGATGTGAATCTCAGCTGAGTATGCACAAGGCAGTA
GTAGACACCCAGGGAATGTCTGCTGGAGTGCATCCTCATGACATGGCCCCAGGATTCGCGGCTCTGAGGAAGACTGGGGTTAGAAAG
AAATGGAGACAGCCGAACCCGGCCCTCATTGTATGGAAAGAACAAGATACGGCTAAAAAGAAGGCATAGCTAATATTAACAATAGCT
AATAGTGACTGCTTTGCTCATGGGGGGTGCATGGCGGGTTTGTTGAAGATCAGATATACTATTGTATATGATAGATGCAGTGCACCC
GTTGTAGCCTCTGCAGCAGATGGCTCATCCCCAGGCAAGAGGATGGAGAGGCAGAGAGGTTCAGTTACTCACCCAGGGTTCCAGCCA
GACCTGGCTAACAGTAGGCTCAGGAGCCATGCAATGAATGCTCCATGCATTACCCAAGAGTGCAGAGTGGCCTGCAGACACTTGCTG
CCCCTTCTGAGCTGGGGTCAGGAACCTGGCTGCCCTGTAAGCTCACTGCTACAGGAAACATCCCTTTCCCCCATGCCAGGCCTGCTG
AGACTCAGGGGTGTGGGGTTTGCTTGCTTTTGTTTCTCCTTTAAATACAAAAGCACTCTTAATCCTCCTTAGCACAGAGTAACACAT
GGAGGCTGGATCCTTCATGCTGGAAGGGACTTGGCAGTGGAGAGCAGAAAGAGACGGACCCAGCAGCTCTCCTCATTCCCTCTGCTC
ATTGGATTTTGGGGAGTGCTAATAATGTATTTATTGTCATTTTATGTGCACTGGTGGTTTGCCTGCATGCATGTCTGTGTGAGGTGT
CAGATCTTGCAATTGTAGATGGTTGTGAGATGGCACGTGGGTGCTGGGGTTTGACCCTGGGTCCTCTGGAAGAACAGTCATTGTCCT
CAATTGCTGAGCCATCCAACTCCGATTTGTGGGAGTTCGGTCCACATGCCAAACAAGAACAACAAGGGGGTGTTCTGCAGTTTAACC
CAGCTTTAACACTCTACTTGAAGCTACATTAAATGCCACTGGCTACTGGCCTGGTCCTTCAGCTCTGTCCCACTTCAAAAGTCCACA
CGGGGCTGACTGAGCTGTGAGTCCAGGCTCCCATGACCCTGGGCCTTGGATTTGAGTCACCTGATGGAATCGAGTGCACAGTTCAGG
GAAACATTTGCACAGTTATTCTTCGGGGTGTTTCAGAGAGTCCTGGTGAGCAGCAGCCTAGAGTGGACTCCAAGGGCAGGAGTTCGA
GAAGGGTATGAAGCTGCCGGGGAGCCTGTGGGGGTGTTACCTCCCAGGTGCCTCCATGTGGTCAGCTATGTAGAAGTCTCCAAACTT
GGTCCTTGGGCTTGGCAGAGGCTTCTTTGTGTAGCACACTTGATGAAAGCGTTTGCCACTGATGGTCAGCTGCTAGGGAGGGTGGGG
TTGGTAGTCTTTCTCCTCCTTGCTCCCATCCCAAGGCTGGCCAGTCAGGATGTGGGCGTTCGAAAGACTCTGACCCTGACTGTGCTG
GAGATGTGACTCAGTGGCAAAAACACCAGCCCGGCATTTAGGAGACCCTGGGCTTAAACGCTAAGTAACAGTAGGCAGCTGTGCACA
CCTTTAACCCCAGCACTTAGGAGGAGAAGCAAGTAGACCTCTGCGAGTTCAAGGTGAGTCTAGTCTACAAAACACAGGCCTGCCAGG
ACTACATAGTGAAACCCTGTCTCAAAACAAAACAAGCTCCAAAGACTCATCATTTTTGAAGCTCACTGTGTGTGTGTTATGTATATG
TGTGTATGTACATATGCGTGTGTATTTGTGTATGGGGCCTGGGTATATATGTATGGTGTGTGTGTGTGTGTGTGTGTGTGTGTGTGT
GTGTGTGTGTGTGCCTGTGCACATGTGTGTGTATTGCTGGGGGTTAAACCCCAGTGTCTTGTACATACTCTGCTATACTCCAGTCCT
CTCCTGGGTTTTAGTAGTTGCATTCTTCAGCCAAGGTGAGGACCAAATGTATTATTTCTTATTATGAATGTATTATTATCCAACTTG
TCATGATAGCCTGTTCTTGCAATCCCAGAACTGGAAAGGTTGAGGCAGGTGGATTTCCATGAGTTTGAGGCTCGCTGGTTACAGAGT
GAGACCTTGTCACAAACAAACATATCTTAGGAAGCAACTTCCAGAGGAGGGAAGTGAGGTTCAGAGAGTGGGTGTCTTAGCTAAGAC
CTCCATCTGGATCCAAGCAGGTCTTTTGACTCCGCTGGGACTCTCACACAGGTAGGCTCCTAGCCTGCCCCTGAGGTGGTCAGGCTC
TTGGACCCCCAGCACCCCCACCCTGCCTCTTGTGTGACACTCCTGCTCACCTGTCCTGGCCACACTAATGTGCTCTTCACTCCTCCA
GCTCTTTGTGATCGACAATGGTGCAGATGACTGGCGGATAGCCATGACCTACGAGCGTATCCTCTACATCAGCCTGGAGATGCTGGT
GTGCGCCATCCACCCCATCCCTGGAGAGTACAAGTTCTTCTGGACTGCACGCCTGGCCTTCTCCTATACCCCATCTCGGGCAGAGGC
TGACGTGGACATTATTCTGTCCATCCCCATGTTCCTGCGCCTGTACCTGATCGCCCGGGTCATGCTGCTCCATAGCAAACTCTTCAC
TGATGCCTCCTCCCGAAGCATCGGGGCCCTCAACAAGATCAACTTCAACACCCGATTCGTCATGAAGACGCTCATGACCATCTGCCC
TGGCACGGTGCTGCTGGTGTTCAGCATCTCTCTGTGGATCATCGCTGCCTGGACTGTGCGAGTCTGTGAAAGGTATTTCAGGGAGAC
CCTGAGCGTGGGATGGGGGAGGGACAAGGAGCAGGGATGTCCAGGTCTAAGGTCTGATGGAGGAGGCTGGAATGGTCTCAGATTCCT
CAAAGGAGAAAATGAGAACAAGAAGAGGAGGGCAGAGCTGGTGTAGAATATTGGAACATTGGAGCCCAGGTAAGCTGGGAAAAACAT
ATATAACTAACGTTTGTTTCTTCTTTTAGACCAAAGTGTCACACCTCCCCCTTTTATAATATTTATTTATTTATTCATTTATTTAAT
GTATATGAGTACACCGTATTCTTCAGACACACCAGAAGAGGCCATCAGATTCCATTATAGATGGTTGTGAGCCACCATGTGGTTGCT
GGGAATTGAACTCCGGACCTCTGGAAGAACAATCAGCCCTCTTAACCATTGGGTCATCTCTCTAGCACCCTGGTTACATCTTTAACC
ACAGTATTTATTGATTGACAAGGTTTTGAATGTTCTCCTCTTAGAGATTAATGATGCTTGCAAATGTGTATAGGGCTTGGAAAAGCC
CTGCAGTGATGAAGCCTATTTGTTTTTGCTTAGTTCAAATTTCCTCATATAATTAATTTTTGCCCACAGATCTTTTATTTTTAATCA
CACACCTCTAGTGATATGTCTCTGCTCAAGGTTTGGTCCACTGATTGACAGCATCAGCATCACCGGGAGACTTGTTAGAGCACAGAG
CTTAGCCCCAGAGTTACTGAAGCAGCAGTGGCCTTTTAGCAGTGTCCTGCTTTGATTCATGTGCACTTTAAAGTTGGAGAATCACTA
TGGTGGTGAGACAGACTGAGGCTTTATGAGGCTGGGCGGTGATTCGGAGGTCTGCAGATGGGAGTGGACGGAGGGACTGTGGTCTCC
CAGAGACCCAGGGCTCTGCCATGACCTGACAGCTCTCAGAACATCCACCCTTGTGGCTCCCCATTAGGATGATACCACCACTCCGCT
GTACCCCTCAGGCCCTGTTGGTGACACCTGTCCCTTTCAAGCTGACCGTGTCTCGTTCTAATCAGATTTCCTGTGTAGCGTCTGCCC
GAGAAGCCGTGGTTTCAAATTGAGTGTCACCTGAATTAGAATAAGGCCATGGTCCCGAGATAGAGCGCTTTGGTGTGGGAAGGCACT
GAGGAAAATCCATGCTGCTTACACAGGTCCTGGTAGCTGTCCTTAGTGCATCAGCACTTGCGATTTGACTGTCAGATCTCCCACATG
CCCATCTCTGAGCTGAGATAAAACAAGGCAGAGAATTCAAGCAGACAGCTGGCTGAATAGTTAGCACATGGGAGCCACGAATGAGAT
TTCCCACTGGCTTTGGGATATTCAGCCAGTAGGTATGGCTTTCTGATTTCAGACAGAGGCATTGTTCTGCCCAGAACATGGTCCTTA
TATGAGAGGAGCAGCCTCTGAGAACCAAGCTGTTCTCTCTGGGGAACAGAGAGCCCTAAGCGACACCACCCAGAGCCCTACCATATC
TCAGCAGCTAAGTTTCTCTTCCAAGCAGGAATCCTTACCAAACAGCTCTTCACAAACCTTTCCTATGACAGAGAAATGGTGCTCTGC
TTCCTACCCACAACTCCTCACGGAACCATCTTGCTCGTTATTTGAGACAGGGTAGAACTTGTGACCCCTCGGTCCTATTTCCTGCCT
CACTATGCTGGGATGGCAGGTGTGCACTACCACGCTGGCTGCCTCATGGGACTCTCTGCTGAGGAATACCAGTCACTCTGCCTTCCT
GGGATTCAATGATGAATGAAGAATAATAATTCCTGCTTTATAGGATTCATTGTTAGAGCCAAGCAAAGCAATCCTTGCAAACTGTAC
CTTCCCACACATTGTGTAATCCCCGGGTAACTCGGGATCTTTGTGGCTGCCTTTTTTAAATAACCAGGATAGACAGACTGCTACCAA
ATGCCCACTGATACTCATCCTATTTTCCTTTCTAATCATCAGAAGCCAGGCTTTCCAACAGTGGTGATTCCATTTGTATAGCCCTTT
GGGGCACTTGAGGTTTTTCTTTTGAAATACATGTATTTCATGAATACTTGTCTGGCATGTATTTCAGATGAATAGGCTGGTTAAACA
TACAACCAGGTACCTACAAGTGAGTTTTATTGTTTATTAAAACTGTCACAAGTGTGGTGGCTTATTTATTCATTTCTGAAGAATAGC
CACATTTTGAAGGGCCTTGTTTACAGAAGTCTTTGGATCTTAGTTGACATTGAACTCCCTTCAAAGCTCCCAAGTTCAGGCTGGACT
TGTCTGGTTAGATGAAGTTAGTTTGCATTAGGCTGCAGTAGATTGTGTTAGTCTCCATTAGCCCAGGTTAGACAAGACTAGCCCTTG
TTAAACTAGGTTTGCCTGTGCTGTCACAGAAGCCCCCACAGTTACTCTTGTTGCAAACATGGCTTCTGAGCGTCAGTTGAACATCGG
GAAACCAAGCAAGCCAGAGCCTCCCATAGTCTGCCCTCTAGGGAACAGACTTTGTGATCACAGGCTCAGAGGTGGCTTAGGCATATG
TGGTGCTGAGCACAGGAACACATCCTGCTACTGCTTCTCACTCTGTGCTGGTTTTGTATCACGTGCTTCATGCTATCCCAGCCTAAC
CCAGCGGCAGACTCAAGGAAGCCAGTCTGTCCTCACCTTAGGGCTGGCTGCTGGAGAGAGCGTGGTTTGAGGTCTCAGGGCTCCTAA
GTGCACAGTCAGGGCGTAAGCATGAGTTCAATTCCAAACTTCAAATTTACCTTGTCAACCCAGGCTGGATCCTGCAGCGACAGGAAA
TTGTGCTCTAGACTGCCCTAGACACATCCCCACTCTGAGGTCTGAGGGTTACTGTGGTGACACAAAAATGCCAGGCTTGTCCTTTGG
TCTTCTCAGATGTCTGTGTCCTTGATCGCTTTGTCATGCCGTTTCCAGGGCTGTCTTTATGCTCAGTGAAGTCTTCATGTGTGTGCA
CATGCGTTTTTGTTTGTTCATGTGTGTATGTGCCTGTGTACATGTGTGGGTGTGCATGCACGTTTGTGTGTGTATGTGTATTTAAGT
GGATAGAGTGCACCCACTCTGCCCTGTGTTGATTTGAACACCAATCACTTCCACCTGGTATTGCGCTGGCTGTGAAACGCCACCTCT
CAAGGGAGCTGCCTGACTCAGGGGTGGCAGGGAGCCACTGATAGGGTATAAAGTAGAATTTAAAGGCCCCTTTACCTCCTAGAGCAG
TGGTTCTCACCTTATGGGGTTTGAATGACCCTTTCAGAGGTCTGCCTAAGACCATCTGCATGTCAGATATTTACATTATAATTCATA
ACAGTAGTAAAATTACAATTATGAAGCAGCAATAGAATAATTTTGTGGTTGGAGTCACCACATGAGGAGCTCTATTAAAAGGTCTCA
GCATTAGGAAGGCTGAGAACCACTGCCCTAAAGGATAACTGTCTAGGGTCATCAGACTCTAGGGGTCCCTAAGGAGTCACCTGAGGT
TTCTATTCCCACACCTAGCCTGTGTCCCACTTCCTGTGCTGATTCTGAGAGCATCCCCTTCCCCTAAGTTTCTGTCTCCAGATCTCA
CAGTCTTTGACTCTTGCTCAGAGTCCGACCTATAGTACTGGGAAACATGTACAGAACTCGGGTAATCAGACAAAGCTCTTCTCTTGG
AATGGCCAGCGAGTTTCTTTTAGATCTGTAGGTGACCCTGCGCTTTCCAAGGCCACATCCTAAGCATGACTGGCTGGGACACTGAGA
CATGGGTTTCGCTGGGGGACTGGAGGATTGGGCCAGGGTGGGAGGAGTATACGGAAAAGATGACCTTGAGCTCTCTCAGTTTGATCC
GATGATGGCTCTGCTACCTCTGATGTTAGTGAAGGTGGCTTGTCATTGGCATTATGCACCATGACAGGTTCTAGAACCTGTCCTGTC
TGCTGTTGCCCCGACTAGGATTTGCATACAGCCCGGGTGGCCTCCCTCAGCTGGACTTATCTCTAGCTGTATACACACACTTCTGAA
GTTGCTATGGAAATCCTCTTTAGCAAGATGTGTTCCAGAAGTCATTTCTCCTATGTGGGGTTGGTTTTTAATGGGACAATAAATGTT
TTTACATCTCCTCCTGCCAAGGACAGACGGGAGTTAATAATGGCGCAGAGGAGCCCTGTGTTGGGAGAGCAGCACCTGGTTGCCAGG
AGGGCAGTAGCAACATTGGAGGCTTGTTTTGCTGGGACCCCAGAGATGCATTACATCACTGCCTCCGGAGCCTGACAGTTGCAGCGC
TCAGCATTCTCCAGGATTCTCTTGGCTGCGTGCCCACCTCCCACAGGCCTCCCAGGGCCACCCCAGGAACCTCCTCATTAAACACTG
TCAAATGCCAAGCAGCAGTCGAAGGGGAACAGTGGAGGCCCCAGGTCCCAGTGCGCCAGGCCAGGGAAGTCGGCTTTCCCTGCACAC
GCTGAACAATATTCTAGCCGCCTACTTGCGCGTGTTTATCTCCTTGGAAGAAGTGGAAAGGCCTCCACAGTTCTGACAAACACAGCC
TGAGTCTTGCTTTGCATGTAAACCCTCTCCATCTTCTCGACTGGCTGGGCCCTTTTGGATTCTTCCCTCCCAGGACTCCACACTCTG
TACACAGGCTGTCTGCCTCAGGGGCTCAGGCCTTGTCAGCAGGTGTTTGGTTTTGACTGTGTGAATTTTAATTTTTTACACAGGCAT
TTTAATGAAGAATTTAAATGTAAAGATCTTGCCTGTAGAGTCAGCGAGAACCAGGCCATTGTGGGGTTAGGGTTACAGCTCAGGGGA
GAATACTTGACTCTCGTGGAGGAGACCTTGGGTTGATTCTCAGAACTGTGGATGAAGAGCAGGAGGAGGAAGAGGAGGAGGAGGAGG
AGGAGGAGGGAGAGAAGGACGAAGAGCAAGAGGAGAAAGAAAGACCAAACTATATTCTTTTCTGTTATTGTTTTTTGTTTGTTTGTT
TGTTTATCTGGTTGGTTGGTAGGCTTTTGTTTTTCAAGGCAGGGTTTCTCTGTGTAGTCCTGATTATCCTGGAATTCACTCTGTAGA
CCAGGCTAGCCTCGAACTCAGAGAGATCCACCTGCCTCAGCCTCCCAAGTGCTGGGATTAAAGGCATAGGACACCAATGCCCAGTTC
CAACCTACATTCTTGTTCTCTGTTGGTGATTGGTGAAGCTCTGAGGCAGGAGAAGAGAGGAACAATGGGCATTGTTCAATGCCATTG
GGCAATGGGCAATGGGTGTGACTTGAGATGGCAATGGACGTGACTGGAGGAAAGCAGTTTGGAGCTTGTCTCCAGGATGAGGCTCAT
CAAGGAAGCCCCCATGCTGAGCTGAGCAGAGCTTAGCCAAAAAGAGCTTAAGAATGGGGCTTGAGACTGTGGTGGTAGTGGTGGGGC
CTTCGCTGAAGCACACACTCCACTGTGGACTGTGGTGTCGGGGCCTTCGGTGAGGCACACACCCCACTGTGGACCTTGCACAAAGAT
AAGTGGGAACTCTGAGAAAATAGTAAGCACTTCTAGACATAGCTATTTTATCTAAGATAATTTGAGTTAAGCCTGTATTTAATATAC
AGGTGTTCAACACACACACACACACACACACACACACACACACACACATGCAGGGTGGTATATACTGAAAATTATTTTACTGTTAGA
AGTCTGTGCTTTTAAGTGTTTGTAAATGACTGGTTGAGGCAACTGGATGATGAATCAGGAGGGATTCCATGCGTTGGGGGCGGGGCG
GGGGCGGGGCTCACCTCTAGTCCCACCGTTAGCTGGGGGAAATGAGAAGTTGGTCTTCTTTACCAGAAACCAGTCAGGGTGGTCCTG
CTCCTCAGTAAGGAATAGAAAAATTACCCCGAAGATGCAAAAGAACATGAATTATGGGGTACCCAAAGCAGCCTTGGAAGACGAGAT
ATGCCTCAAAGTGCGAGAAAGAAGCAGTCTCTGTTACGAAGAAGACAGCAGTCGAGGAATGAAAACACACCTGCACGTGCTCAGAGC
ACCGACTTTGTGAAAAAGAAAATCCCCATTCGTGATGATGACTTTCTTCAAATGTAGAAAGTGGAGGGCCAGCTAAAAAGCTTCCCC
AGAGGGCCAGAGAGAGTGCTCATGGGAGTTAGAGCTCTGGAACCCATGTGGTGGCATGAGAACCAGCTCGTGCAGGTGTCCTCTGAC
CTCCATGGCTGTGCGCTTGCCTTCCTCCAGAAATATAAATGAATGAGTGAATGAATGAATGAATGAATGAACAAATATCTTAAAAGT
TGTTTCAGAATCTGAGAGATGAGTGGAAGGTGGTTAGCACAGACAGGGCCCATGAAGTTGAAGTTTGGACAAAAATATAGTCAAACA
TATGCATGAAAAAAAAAAAACAAAACTTTTCCAGAATGGCTGACAGATAGGCAAGGAGTTGGTAGACCCGGAAAGGCCAAGCAAGAC
ACCACCTTCTTTCTGATCGGAAGACCAGTAGGGTCCACCTCTAACATAACCACTACTGTTTCTGGAATCATAGCAAATCATAAGATT
AGCCAACCTAGAATCATAGAAACAACAAAACTGGTTTCCTCACACCCGCTAAACTTTAAGAGCCAGTAGGCCATCTTGACAGAGTCT
GCAGTCTGTGACGTGACCCCCTCTGCCATTCGAACGAAACAACAAGACTCATTATTAAGATATTAATAATGGGGGGCTGGAGAGACG
GTTCAGTGGTTCAGAGAACTGGTTGCTCTTCCAGAGGACCTGGGTTCAAATCCCACCAACCACATAGTAGCTCACACATGCCTATAA
TTCCAGTTTCAGGGATCCAACACACTCACTCACAAATATATGCAGGCAAAACGTCAATGCACACAAGATAAAAATAAAATTTTAAAA
AGATCTTAGTAATAGACTGATGTGAAAGCCAGAGAGATAGACAGAGCTATGCATGATCGCCAAACCAAATTTTAATGTCAAAGGAAA
AGTAGATTCTACAGAGTAGGTGCGTGAAAC2AAAACAATAATTCAACAATAGTTAGGGGATTACAAATATTTAGATTAAACATTTAA
AAGGAGAGGGCTTGAAGGGTTTAATTCATCATCTGATGTAGAACATTTTAATCCTTAACATTCAAATTCAGTAATTAGAAAAATATA
GTTGAGATCATAGTTTAAACCTTAAGTTTACCTCAAGTTAGGATTTAAAACAGGATTTATTTGTTTATTTATTTATTTATTTATTTA
TTTATTTATTTATTTAATTTTACATGTGGGTATTTGCCGGCATGCGTCTGTGTACCACCGATGTGCAGTATCCACGGAGGCCAGAAG
AGGGCGTCAAATGCCCCCTGGAACTAGAGTCATAGACAATTAGGTGCTGGAATCAAACCTGGCTTCCCAGAAGAACTGCTGGTGCTC
TTAACCACCGAGCCACCTCTCCAACTCCCACAGTAGGACTTAAAAAAACATACAATAAGGGTAACAGTAGCATCCCCTAAGAGTTGT
TCCACCCCAAACTATCTCATCTGTCTGAGGCCTGGGGGCTGTTACACCCCATTCCATGTAGGATGAAGCTGAGACCTGAGGAGCTGA
AGCAACGTGCAATTCCCAAACATCAGATCTACCAAGCTAAGGCAGAAGAAACAGGGACTCAGATGGGGAAACGTAAAGACAAAACAC
AAACACAAAAACCAAATGACAGAAAACCACATCTAAAAGGTATAATGATCAACATCAGTTGAGTTACCTCCTCAATTAAAGGACAGT
GTCCTTTGCTTTGATAAGATTTTAAAGCAGAAGTTGTTTTTATGCTAATTTTAAAAGAGGAACCTAAGGCAGAAGGGACCCAGAAGA
AAGTAAGAGCGGAGAGTTACATAAATTCAAAGCAGAGGGCAATGGAGACAGGCTGGTTTCATAGCACAAATTATTTTGTGGTTCAAA
GGAATCATTTAACATTTCAGATGGGAAGGGGTGTAGAGATGGCTGATCAAGTAAGTAAAAACACTGGCTGCTCTTTCAGAGGACCAG
GGTTCAATTCCCAGCACTCCTGTTACAGGATATCTGAGCCCCTCTTCTGGCTTCTGTAGGCACTAGACCCACCAGTGATACACAGAC
ATACATGCAGATAAAACACCCATGCATATGAAATGAAATGAAATGAAATGAAATGAAATGAAATGAAATAAAATAAAACCAACAAAA
ATACCATAACATAGGGTAAACTTTTGTCCAGAAAGGGTGAAATTGAATATCTGGGGGCTGGAGAGAGGGCTTGGTGGTAAAGTCCTT
GTCATGGACGTGGAGGACCAGGCTTTGGTCCCCAGAACCCACATAAAAGCCAGGTTGGTGTGTAATCTCATTGCTCAGAGACAGAGG
ATCTCCCAGCAAGCTGGATAGCCAAACTAGCCCTAATGGAAAACTCCAGGTATACACACATGCACCCCAGCGCGCACACACACACAC
ACACACACACACACACACACACACTCTTTTAAAATAAGAAAGAACACCTGAAATACTAAAAGTTTTGATGGAGAACTACCTTAAGAC
TAATAACCTGACCAAAGATGTACTCAGATGAAAAGTACTCTCAAAGTTTTCATTATTAAAAAGTAAAAAACAGTGGAAAGATTGTGC
AGTAGTTAAGAGCACTGACTGCTCTTCTAGAGGACCCAGGTTCAAATCCCAGCACCCACATGGCAGCTCATAACCACCCTTAATTCC
AGTTCTGGGGGATCTGACTCCCTCTGTGGCCTCTCTGTGTCCTGCACAAATGTGGTGTACTGATGTACATGCAGGCAAAACACCCCT
ACAGATAAAATAAAAATAAATACATATTTTATTAAAAGTGAAGAAGGAAGCCAGATACTGTGGAACATGTCTATGTATATGTGTCAG
GGATCTGAGGCAGGAGGATTATCTTTAGTTTTAGGCCAACATGGGTTACATATTGAGTACCAGGACTTTATACCAAGATCCTGTCTC
AAAAAGCCAAGAAAAAGAGAAGAAGGAAAAGAAATTATGTTTATTAGAAGGATGAAACAAACCTTAGATGGAGAGCAGACTAAATTT
ACAATGAAGAAACGGGAACTAATAAATTTTTAAAATGGGATTTATAAATCTGAAAGCTGACAAGTTTAATAAATTTAAACAATTTCT
GCTGTATTGGAAAACAAAATTAGAAATTTTAAGGGAAAATAATGAGTGATTCATAGGGCATTTTAATCAATAATCGAGAGCAGCTTG
GCTCATACAAGAGAAGCCCAGGAGGGAACTTTCCCTCTGGATATTAAAATGTATTATAAAACAAAGAATAATTAAGTCCCTCAGGCA
TTACCCATTACTAAAGAACCCAGTAATGACAGATCGATGAAATGCAATGGAAAACCCTGAGATAGAGCCTAGTGTGGAAACCCAGTC
ATGGAGAAAGAAAACCAATTAGGAAGCAATAGATTACTCAATAAACGTTCATGGGGAAATTGGAAGCTTGGATGAAATATCAAATAG
GCACGCCACCTCTCAACAGCCAGCAAAATAAATTCCAGGTAGATTAAAGAGTCGGATAGATATGAGGGAAGAAATACGAGCAGGAGC
TAGAAATCAAAGTGAATATTTAATCAATCCTGGGGTGAAGAGAAATGTTAGGCAGAACATCTCAGGAGAGAAGAGCTGTGAAAGGAA
GCAGGCATATTTGATTACATCATAAATACAAGTGTAGATTTCTGTCCTTTAAAATATATAAACCAGGGCTGGAGAGATGGCTTAGTG
ATTAAGAGCACTGGCTACTCTTGCAGAGGACCTGAGTTCAATTTCTAGCACTTGCATGGTGGCTCAGAGCCATTTGTTACTCTGGTT
CCAAGGTATCTGACACCTTCTTCTGACTTCTACAAGCATTAGGCATGCCAATGGTGCACAGACATACATGCAGGCAAAATACCCATA
CACACGATATATATTATTATTTTATTTACTTATATTATAAGCATACATGTTAATTAATTAATAATTATTATATGTAATAAGCTAGAA
ATGGTGGACCTTCTCTGTAATCCCAGAGCTAGGGAAATGGAGACAGGAGGCTCAAGAGTGAATTTAAGATCAGCCTGGGATACATGT
CTCAAAAAAAAAATCTATTTAAAAGCAAATGTTATATTTAAGAAAGATATTTGCAACAAATGTGACAAGAGAGAAATATCTCATTGT
CCAAAGCAGTCCTGGAGATCAGCCACAGTCCACCTCACAGCTGAAGGCAGGCACAACATAGAAGGCACACAGCGAAGACACCAACAT
GAAATGCAGCGAGTCAGCTGTTAACCCTGAGTTTGCCGCAACTTGAACAGTGGGAAGCTTTCACTTGTTGCTGTTAAGGTCTTCCTG
TTGATGGTGGGGGTGGGATTGTGTCTCACACTAGAGCAAACAGGGAATTTAGGACCATCTTTCTGGAAACAACATTGCTGGGAAGCC
CTTGACGGTTTATGGCTTTAACCTACTAAATATCTTTCTAGAAAGTTCTTTGAATCAAAGACTCAACTGATACTCGAGAGTCTTCAA
TGTAATGTTTATAATATCAAAGACTCAGAAATGAAGTGAACATGTAGGATTTAAATGCTTAAATAAAGGGGCTGGAGAGATGGAGGT
TCAGCAGTCAAGAGCACTGGCTGCTTTTTCAAAGGATCTGGGTTTGGTTCCCAGCACCCGTGTCAGCACCCGTGGCTGCTGACAAAC
CTGTAACTCCAGTTCAGGGGATCTGATGCTCTCTTCTGGCTTCCCTGAGCACTGCATGTGCCTAGGTGCAGGCAGGCAAAACACCCA
TGCACAAAAACTAAGACCTAGAGTCTTTTAAAGCATGTTTGAATAAGGCTTGTGCATGCCGACAGCGAGTTCTTAGGCAACGATGTT
ACAAGTCCGTACTGATGTGGGAAAACNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNCTAATGGTATTTTTGTTTGTTTGTTTGTTTTGTTTTGT
TTTTCGAGACAGGGTTTCTCTGTGTAGCCCTGGCTGTTCTGGAACTCACTCTGTAGAGCATTTTTTACGTATATATGTGTAAGAAAT
GGGTTTGGGGGCCACAAGAAAATGACCCCACAATCCAAAGCCTTTCAGTAAGGTGGTGAGTTTTACTTGTTCGGGAGGGGTGTAGCC
AAGGCAAGCATGCAAGCACACTCGGCAGCAGGGCTGCCTCTGCTCTCTGTTCCCTAACACACAGATCCCATGTTTCCATTTTTCTGT
GATTGGCCAGCTTAAAAAGCCCAGTTTATAAGCAGAGCTCAATCTCTTGCCAGGTGCAACCCTGAGCAGGCACATATTTTGATTTCA
GGAAGTGCTGACTCTGGAGGAAATTTTAATTTCTGCCAAGTGACTGGTTAAAAAAAATTAATAGGGTTTTGTCTTATTTTAACCCTT
TGACAGAAAGACAGTGCAATTTGTTTATAATTTCCACACTATATACTTTTGTTGTTGTTGTTGTTGTTCTGTTTGGGTTTTTTAATT
GTTGTTATTGTTCTGTTTTGTTTTTTGATTTTCCACACAGGGTTTCTTTGCATAGCCCTGGCTGCCCTGGAACTTGCTTTGTAGACC
AGGGTGGCCTCGAACTCACAGAGATCTACTTTCCTTTTCCTCCAAGTACTGTGATTAAAAGTGTGTGCCACCACAACCACCATGACT
CAGATCTGCATTTTTAATGGAGAGAGGCAGCTGGCTTTGAGATTTGGACAAATGTCATTTTAGCCTCGGCACGAAGCCTTGTCATCA
ACAGGGCTTGGGGAGTAAGGTCGGGTGGTACAGTTTCCACATTTAATTCCCTGGAGGATCACCAAAGAGTTCCTTCAGAAGGGCTCC
CCTTCCTATCTAGCAGCTAATGGCCTTAGAGATAGGGACAGTGACTGGGCAGGAGGCTTATGGGTCATCTCTCTGGGGGGCCTCCCT
GGGTGGGCTTGTGACACTACCACGGAGTAGGTGGGTGAGGCTCACAGATGCTAGCTTGCTTGGTGGCAAGATAAGGGAACACTGCAA
GTTAGAATGCATTCAGAAAGCACAGAAAAAGACGCTGTGGTCTTCCTTGCAAGAAAGGTCCTGCTACTGAGCTGTGGGACCTGGGTG
AGAAACCTTTCGTTCTGCACCTGCCTGGCCACAGGGGCAGCGGCACCCAAATCTCCTTTGAGCCCAGAGCAGTGAGACCCTGGCAGT
GTGCTGCCATGCCCAGGTTCAGCCGGCTGAAGCCACGGTGACTTCAGCCACCAGTCCCAGGGCCATTTCTTCTCCCTGTCACTAGTG
ACACTAGTCTCAGGTCTCTGTTTTGAATCTCTTCCATCTTCACTTTTTTTCACAGCTGAGTTTCTCTTTCTGTTATTTCTGATTCAC
ACTTGTTTTGTTTTATTTCCTTTGCATTATGTTTCTGCTCTTTGGCACTTAAAGAGCAAAGTTGCATATTTGTTACTGTTTTCTTCA
TACTTTTATTTATGTTAAATATACATTAATTGTAAGTTGCGTTCCAATTAAGGGATGGCCTCTCTTAGAAGAGTCTATAAACGGGAA
TCAGGAAGGTTTCCTAACATGGGCAATGCTTAGTTCTGGGTACAGGACAGCACCTGAGGGTATAGTGGGAAGCTCTGCGGTCTCTGC
ACACCACCCAGCTACTCCAGCCTGTGGTACAACTGTGAGGTTTCACTCCTCTGTCTTTGGCAAGCATCTTTTCCCTCCAGGATGTCT
GCTTTGGGGTTACCCACAGGAGTGGGGGGTTGTACAGCTTTTCTCCAGCCACAGTGCAAGTTGCTTTTTTTCTTACAGTGGATACTA
AGGACTCCTGTACATGTCCAGAAGGCCTGGGCTCTGAGCAGAGGGGAGGACTCAAAGTTTGCCAGGCTGGGGCTCCTTAGACAGTGA
TGGAGGAAAAGGGTGAGGTTCTGTGAGCAAAAATTACCCATGTCTGTCTCCCAGCTCAGGTTAGCCCACACATGAAATCTGTCTTCT
GTCGAATTTCCCACTTTCAACCCTTGGCTGCTTTGTTTGGTCCTTCAGACCCCACCGTGTCCTTCATCTCCATGGGAGGGACCTTGG
CAGCAGTGGATGGCCGTCTCTGCTCCACAGGGACTGAGCTCCGTGTTGAGGGTTGACATTCATTGCTATGGAAACCTTTCAATCCTT
TCAATAGGGGAGAGCCATCCGAGCCACGGAGGGACTGGCACCTTCACCAGTGCCTGACAAGCAAGGAAACCTCTCAGAGAACAAGAG
GCTGGGGGTGGGGGAGGCATCCCAGCAGGCCAAGCAAGGGACACATGTGACTTAGTCTTACATTCCAAATACATCCTTGTGCCTGTG
TGTCCTCATGCACTAGTGTCCCAGGCACTGGGTCTATGCTTGGACTCTCACATTGCCCTAAATTCTCTCTGCTTTACTGTTTTGCTT
TTTAAGATTTATTTATTTAATGTATGTGAGTACACTGTTGCTGTCTTCAGACACACCAGAAGAGGACATCAGATCCCATTACAGATG
GTTGTGAGCCACCATGTGGTTGCTGGGATTTGAACTCAGGACCTCTGGAAGAGCAGTCAGTGCTCTTAACCTCTGAGCCATTTCTCC
AGCCCTACTTCACTGTTTTTAACTCTTGGTTCCCCCCTCCTCCCCCCACCCCCTACCCCCGGGAACTTTATCTGTTTTGTTCTATCA
TATTAGATTTTTGACTCTGGCAAAGAAATTAGAAACCTTTAAACAAAATAGTAAAATGTTGGAGAGGTAAAAGAAGATGGAAAAAGG
GTCAGTCTAAGCAAGAAAGGAGTCTGAAAACGTCAGGGGCAGAGGCTCCTCTCTTGTTACCCTACTGAGCTGGAGGCGTGGCTTCAC
TTCTCATTGAAGGCGCTGGAGCTCCATCAGCACATCTACCTTGTAGCCAGCAGGAGGAAGAATGTATGCTGCTCTCTATGCAGGCAT
GCTCTGCGCTCTATGGAGCCATCCCACTCACACTCACACTCAACATTTCTGGGGCTGGGGAAATGGTATCTTTATCACTTATGTTAA
TAGTCCAGGTTTGCAAAAATGGACTACTGGGCCACAACTGCTTCCAAAGTCAACATTGTTAGTTATCCTGTGTCCCTGCCAAACCAA
TCCAACTCTCCACGAGGGGTTTCAAGCTCTGGTTCTTGTCTGTAATTCCCCTCAGATTCCCCTTTCTCTACACCTGCTGGCCCGACC
CCTGCTGCTTCCCTGTTTATAGAAGCTTCAGGTTCTGCTACACTCTCGTACCCTTCAAACGCTCCCCGTGTCCCCAACAGAAGGTTA
ATTCATACTCTGCTTAGAGTCTGCAGCTTCCACTGCCCAAGGCCAGTGACCTATGATTAACTGGCTGAGGCCTAGCAGTGTTGGTCA
TAATGCCACAGATCTCTTCATGGAAACTTTATTGTAGACAGTGAAAAACATTGCACAGGAGTGAGCGGGCCTGGGCCCCAGCTCCCA
AAATGGTGCCGGCCTGTGTTGCAGCATCCATGAGGTCTGGGAGGCAGAGGCAGGGAGACTGTGAGTTTCCAGCCAGCCAGGGCAGCA
TAATAAGATCCTGTCTTTAAAAAACAAACCGAACAGGACAGGGGCTGCTGATTATGGCTCAGTGGGAAAAAGGGTTTACATTCTAGC
CTGCCAACCTGCATGGCAGAAGGTAAAGAAGCGAGAACCAAGTCCTGTAAGATGTCCTCTAATCATCCTTCAAATGTGCCATCATGT
GTATACACACGCACACACACAGAGGCACACACCTGTGTACACATACGCATCCATGTGTGTGAGCACATGCATGCATGCACACATATG
CTCATTCACACACCATACTTGCAAACAACATACACACACATCCATGTGTGTGAACACATATGTGCATGTACTCACATCTCTGTACAC
ACAGAGAGATGCACAATACACATACGCATGCACGTGTGTGAATGTATATGTACACATAGCACACATGCATTGTGCACACACACACAC
ACACACACACACACACACACACGAGGGCAGGGAGGGGAGGAAATAGAAAGGAGAGGGAGAAGAAAGAGGCAGGAGCAGAGAAGGTGG
GAAGAGAAGGGCGAAGAAAGCGAGCTCAGCCCATTGCCTCACAGAGCCTCTGACTGTGCCTGGCTCTTAGCCAGCCCCGGACCTTCG
CCCTGCTGGGCCCATGTTCCTGTCTACAGATGGGAGTCAAACCTGCCCTGCTTACACCCTAAGTGGGTTCAAGAAATGGAAGAGCCT
AGAAGCTGGGAACCCCAAAACACAGCTCACATGCTCTCCTGCTACTCCTGACCCACAGAGCAAGTAGGCAAGATGAGAGCAGGGGAA
CCCCATCTGTGCATAAGGATGCAGGGACTCTGGCAGCAGAGGGCTGAGGCCTGGGGTGCTGAGCTGGGGAAGAGGGGGAGGGGAGGG
GAATGAGTTAGCTTTGTCTTGCATTCAATCCAAGTGCTTATTTTACAACTTCTTGAGGGCATGCTGTCTGGTGTCTCAAAAAAATAT
GATTGGTGTGATTTAAAAAAAAAAACAACAACAATAGTGATTTTCATAAGGGAGCAGATGGAAATGGCCTCCTGTTCTTTGGGGGGG
ATGGCAGGTGATTGGATAGGCTTTTCCTGGGAAATACATACCCAAACCAGTATAGTTGCCACTTATATTGTCAAACTTACAGAGTCA
AATGAATGCTGCAGTTGCCTGGCAGTAGTTTTTAATTAGCTTTTTTGTCAAATTTAACACACCATTGTTCTCCTTAGCAGTATTCTT
AATGAGCTTTGCCCCTGTTTACAGTTTCAGTTATAGATTTTATGTAGGGAATAGACATATTTTGAGGTTGTCCATTACTTCAGAAGT
TAGACGGTTGCCTCCTGGGATTATCAGCCTGTCACTATGGGACAAGCTTGTCAAAGTGCAGCACAGGGAGAATAATGGGACCCAGCC
AGACTCAGAGGAAGTCTCAGAGGAAGAGAGAGCCCTTGCTTGCCAGTGCTTGCTGCAGTCTCTGCCATGATGCTGCCTCCTCACAGC
GGCCAGTGATCTCAGTCACGCTGCAGGCCACCTCCATTTCAATGATGGAAACCCGGAGGCCTGAGTCAGGGCCCCTCCCGTCATGGC
TGAAGTTGTCGTACGTACATATGGTCGCCTCGGATAGCTCTTGGCAAGTCCATCTCACGGGGTCTCCCAGAGCACTGCCCTTGCAGG
AGCCAGGGCTTGCTCTGCAATAGACAATGCAGTTCTGTGCTCCCTGGTCCCGCTGTCATCTTTGGCCTTTCCCAGCACTGTGTGGTA
AAGGGAGTAAGACAGGAGTCATCCTCCCACTCTAGAAATGTAGAGGCTCAGGAAGCAAATGCATAAGGAGGGCCTGACCTGGCTGGG
GTCCCCTGGGCAGCCACGTGTGGTATCTTGTGTCACAGAGCCCAGGGTGCCTAGGCCAACGAGAAAATAGATGCCCCGTGTGGATGC
TGGCTGGGGCCATCTTTATGTCGGAATGTAATTTTGAGTCTTTGAAAGTCAGTGGAGAATCAGGCCATTGTTCAATAAAAGCACTTG
TCAGTCACCCCCATCATCTTCCTTAATTAAACCTCATCCCCAGTAGACAGGATCAGTACAGAAGAGAGGAGGCCTTGCAGGGAGCTG
CCCCCAGCCAGCTTCCAGGCTGGGTTCCTGGTCTCCCTCCATGTGTTGAGTCATTCCTTCCTCTTCTGCATCCGCTTGGCTCTGCAC
ACCTGTGGGCTCACACCTTCCTCCCTGGGGTTCCCCCAGCTCCAGCACCACCACCACCACCACCCCCCACCCCCCACCCCCGTCCCT
CCACATGTCCTTTGTTCCATGCCTTGCTGCTCTCATCTGAGGGTTTCCAGTCCTGTGAAGTGTCAGAGGTGGGCTGTAGTTTTCAGA
CATCTTTGTTGCCTAGCAACATGCTACAAGCACCTTCTTCTGTGTGGCTGCTTCCTCTAAAACAGCTGGGGCCAGGGAATGGCGGGA
GGGGTGACCAAGGAATTCTGAGGCCTCTCTGGAGTAATGATGGGGAGAGGAAGGAAAGGGACAGACAGGCAGACACACCCGACTGAC
TGACTTTCTTGCATAACTTTGAGTGTAGTCATTCCCACTTTTCAGTGGTGTAGCTGCCAGAGTCTGCTTTACTGTCCTGTCATAGAT
CCTTGCAAGTGGTCAGTGGTCAAAGTCCTGACTTGGAAGCCATCAGAGACTCTTGGAAGTCACTACTGGCTTGGGGAGGCACCTGCA
GTGACTGCCTAAGGCAAAGCCAGTCTGAAGGCTCCCGACTGAGCTGGCCTCTGGCTTCGTCTTGTTTCCTGAGATGCCATAGCCCCC
CTGTCTTCTGCTTGTGTAGCCAGCCCAGCGATAGTTGCCCTGAGAGTGGGAGGGGAAGATAGGGCAGGAGAGAGAGTGGGATTTTAA
AATGAAACAGGGGCTGCGCAGTGGTAGCACGTGCCTATAATCCCAGTACTTGGGGGGCAGAGGCAGGTGGATTTCTGAGTTCGAGGC
TAGCCTGGTCTACAGAGTGAGTTCCAGGACAGCCAGGGCTATACAGAGAAACCCTGTCTCAAAATAAACAAAATAAAATAAAATAAA
ATAAAATAAAATAAAAATAAATAAATAAAATGAAACCGGCCAGAGAAAGGGCTCAGTGGGTAAAGGCATCTGCCACCAAGCCTGAGT
GTGATCATGTGCAGATTCAAGACCAGCCGCTTATGAATCTGTCTTCTCAAAGATGGCCTGTAAGGGCCGAGGGTGCTGTAGATGCTT
GGTATTGCTCAGTCTGCCCTGCTGGTGTGGGCTGCTGGGTGGAAAGCAAGACAGTGCTCCAAAGATCACAGCCACTCTGAGATGGAG
GGGCCCGTACCCACAGCCCCCACCCTTCATCTACATGAGGACAGTAGGCCCCAAGTGATAGGTCCCAAGCAGCACAGATCACACCTT
CCTCCAGGCACTGCAGTTGACAGAAATCTCTGTGGGTCATCTTATCTTTGCCCTGCCCCTCATTTTCTTCCAGCAAGCACCTCTTAG
TTCAGGAGGCAGGGAAAAAGAGTTCTAACAGTGGCCCCCCTATGTGGTTATAGAAGTGGCCTGAGGTTGTGGCCTAAGTCCCAGCCA
GGAGCCCTGCTTCCAGGGACTGCTCTCATGGGTGGTAGGGTGTGCCATCTTTGAAGACTTAAAAGCCTATCCAGCAAGAAAATTCCT
CTTGAGGGGCGAAGTTGGGTGTGATTGGCTCTCTCCATCCTTTCCTTCACAAGACTGAGCTTCAGCCTCTGAGAAGTATTCCAGAAC
CTTCCAGCTAATCATACAAAGGGAAGTTTCAGGGACTTGGCAGTGGCAAGGGCAGACCTGAGCAAGCTCCTGAGGACTTATGGTCCA
CCTCCCCAACACTTGGACACCCTTTGCTTGAAAATATGTGGTGGTCAGGCCAGTGGGAACACCGAAGAAGGAGATAGCATAGTCCCA
TACCTCTGCCACGAGGGCAAAGGCTAGCTCTGTCCTCTGCCTGGCCCAGATGCCTCTACAGTGACCTCTTTCTCTCTCCATGGCTTT
CTAACAGCTGCAAACTTAGCCTCCTAGAATTCTCCCATCCACACCCCCAGCCTCCAGGGTTCCTCTTTAGCACGCTACTTCTCAATG
GCAGGGGAGGTCTCCCATCCAAAGCTCTCACCCCTTGCCCTTGGGTAAATGAACAAGAAAACCCTTTCTAGGGGCATCCCTCCTGTT
TCTGCCTGTTCTTGGTCACAGAGTGATCTCCACCTCCCTCCCACAGGAACCCTGTCTAGGTGAGACATTTGCTTGCCTGGCTGCCCA
CTTTGCATTCTCTGAGAAAAAAAATCCCCTTCTTTCCTTTGGGGCTCTATCCAGGACCAGTGTCTCTGCAGGAGTTCCCTCCAGGGA
GTGCCATTGAAGCCTTTGTGAATGTCAGTTGCTGCGGCTGTATATAACCCACGCAGCTGAACAGGACAACCCTAGCTCTAAGTTACA
AGGGTTCTTTCCATATAGAGATTGAGGCTCAGAAGCCAGAAAAGGGGAAGTCCCCAGGAACCTCCCCACCCTACTTCCCTGGGCCTT
TGTTTTTTTGGAATCATAATATGGTTAAGCACAGGGCTGGGGACTTGGGCAAAGCAAGCCTATACTCAGTTTAACTGCTCAGTTGGT
GTTTATTTATTTATTATTTTCTTATTTTTTGAGGTGACAAAGGTTGAGCTCAGGCCTTGCAGATGCTAGGCAAGTGCTCTGCCTCTG
CTTTGTCTCCATCCCAGATTTTGTGTTCTTGTTTGTTATTTTAGTCCAAGTCCTGCTGTGTAGCTCAAGCTGGCCTTGAATTTACTT
ACTATGTAACCCAGACTATGTTCGAATTCTAAGTCCTCCTGTCTCAGCTTCCTGAGTGTTGGGATTATAGCGTGTACCTCCATCCTC
AAGCAAATGTTTTATTTTGTAAATATCTCCTAATTAGGTTAAACTGAGTTTCCAATCAGGAAATAAGAAATGATGAGAAATCATCTC
TCATATCAAAGAGTGCATACAGTTGGCACACATGACTCAGTGCTGGTACTCGATAAATGGTAACAATGGTTATTCTTTCTTAAAATA
AAGCTACCACCCGGCCCTACCTACCTCATAGAGATAGCTCCAGAAATAAAGTGAGAAATAGAACATTACCATGATTACAGAGAAAAT
ATTTTGCAAGAAATCCAACTACTTTTATTTTTGTCTCTGTCCAAGAGGACTCAGCCCTCTGAAGCCCCTTTGCCTACAGCAACACTC
TGTGACAGATGGCAGATGTCAAGAAAGGCTCAACCAGGAGAGAGAAAACAACCAGACGCGGAGAGAGGCCCTGGGCAAGTGGAGTTG
AATAAAAATCAAAGACAGAAGAGGCCCATAGCGGAAAGAAATGAGACAAACTTGAAATGCCATTTCTCCCAAAGCCACAGTTGATTC
TTGCCTCTTTCAATAGTTTGCCTGGTGCCACTGACCTGGCTGAGGGACTGGGCCACGTACAGATGGGCAAGGCCTGTGCCAAGAAGT
TACAGGGAGCTGACAAGCTCAGGGGACACTTAGTCAGGAAGAAAGTCTAAAGGAGGTGATGTCTTCTGTCCAAAGGGGTCTCCAGAC
AGGAAGAGCAGTGTGAGCAGCCTCCAGGAATGTGAGAACAGGCCATGGAGAAGAGGCAGCTGTTTGCTGAGAATCAGGCCTTCCTTA
GAGCCAGAGACGGAGGCGGGAGTCCCAATCTAACAGCACAGTCCTCACTTGGTTCTTTAATCAAAGCCAACAGTTCCTAGACTGATT
TCCTTCCTTCCTTCCTTCCTTCCTTCCTTCCTTCCTTCCTTCCTTCCTTCCTTCCTTCCTTCCTTCCTTCCTCCTCCTCCTCCTCCT
TCTTCTCCCCCTCTCTCCCTTTCTTCCTTTTGTTCTTTCTGTTTTTTCTGGTGACAAAGGAGAGCTTCTATTCAGATCCCTGGCTTC
TGCTCCCCATATGCCTCAGGTGTAAGTCATTTCCATTATCATAGCTCTGAGCTTCCTTCCTATGGAGGAAAGCAAGTCAGAATCCTG
AACAAGGAAGGTCTTTACAGGGCTGGGAACCAGCACAGCCAGAGGGCAGGTGCCTCAGCCTTTAGGAGTCTCCGGAGGACATCTCTT
CTTCCTTCTCACTTTCCAGTGGAAGCTGTTGTTGGACGTGCCTGTGTTTTGTTTCTCGGCTGCAGTTTAGAGGATGCTAAGAAGGCA
CAGGCTGACCGTGAGGCAAGGTGCCCAGTGGGGGCACTGCTCGAGTGATGTGCTGCAGGACTGTGGAGCCTAGATCCGCAGCATGGC
TCACTGGGCACACAGTGATGGTAGAACAGACTCAAGAACAATGCCTGGGTCTTAGAGACTGGGAAGTGGTACTGCTTACGAGAAAGG
GAGACAAGTATGGAGGGGATCTGGGCATCAAAGTAAGGCTTAAGAGTAGACTCAGAGATCAGTTGATGTATCTGCTGCCCCATCCCC
ACTCCTAGGTTGCATGCCAGCAGACAATGGCTCTCCTCCTGGCTCCCCTTCCTACTCACTTTTTCCTGCCCTGACTCCTCAGGGGCG
CCTTGGAGTTGGGACCTTGTCCTGACCGAATTTTCTGCCCCCAGTATCTGATCACCTGATATCTGGTTGGTGTCCCCATGGAATGGT
CCCTTGTGGAGGCACGAATGACCTTTTTTTGAGAGTGGGGCTCCCACTCTGAGTCCACCCAGGAGGCTGTGTAAGTGAATGGAAAAC
ATGAGAGGGGAAAAGAAAGAGGAGGGAAGAATGACAGAAAAGACAGACAAGAAAAAGGAGAAGGCAAGGGAAGGGCGGCGAAGGAAG
ATGCATGGTTAGGGAGAGGCAGCCAGCACAGGCCCCCGTGCCTCTCACATACTAACACCCTGAACTGGTGATTTGGGGAGAATGTGT
CTTGACCTGCATAGCTTCCATGGCCTACCTCATTTGTCTCCTCAGTTACCCTCCTCAGCTGGGCTCTACCATGTCAGTCATTCTGGT
GGCTCCTTATAAGCACACTGGTTTCAGAGAAGGTCCCTTGTTCCATATGTGTAGGCCCATGTCTATACACTTGTCTCAGTTGAACAT
GGCTTTTGTCACAGTGGCATTGTTTGGAGTCTAGCTTCATGATGTAGATACCAAGTCCAGCTCACTCTTCCTCATTGACTGAATGAA
GAGGCTAACTGAGGCCAGGATGTGTGTGAAGAGCTAAGTTCGATGGGAGGCATGTTCGATCTCTCCTGTAATGGTCCCACGAACCCA
GGAGAGCTGCAGTGCGCTCCCTGTCAGAGGGAAAGCCTAAACCGTGGAGAGACAAGCACCCAGCACAGCTGGTTGCGTGGCTGGTGC
GGGGAACCTCCTGATGGTCTCTGCCCACGCCCTCTTCCCCACATACAGCCTACTGCCCTGGAAGAAACCTCCCCAGGAATAGCGCTT
CTACCAGGCATTGGTTTTCTCCATTTCCACACATCCTGTGTAACATTTCTCAATCATAAAACATGGAAATATTTTTTAATAAGAAGA
AAGAAACACTGCAAATTGAGAAAGCGTTGCCAGATGAGCCATCTGGTTTTTGATTTAATAACCATCCAAAGAGGTGGACAGCGCTCC
CTCTGAGAAGATGGACTGTGGAGGGAACAGCAGCTGGCTTCCCTCACACGCTGCTTCCTCTGGCAAGGTTGGAGTGGGGTTACGGGA
GTGGAAACAACCAGAGATGCAGGGAAGCTCAGCTGGTGTCATCTAGATGTCCCTGGACTCTGGTGGCTTCCCTTCCTCGCCTCTGGG
CACATGTTGTTAGTTCTTAGGCCTAGAAACAAAGAGCTAGACTTAGATGTGCCTTGGAAAGATCCATCTGTCAAAGGGGAGCATCGC
TATATCTCTAAACTCCAAGTTAGCTCTTGAAAAGCAGTACTGGAAGTGACTTTTGCCTGCGCCATGACAAGTGTTCTCCAGGCCTCG
TGCTCGGCCTTTTAGCTCACGTCTGAAGACGTTTAAAGAAGTAGCTGACTTTTGGGTGGGGTAAGCAGGAATAGTTCTAACGTCCTT
GAAGCCATTTACTTTTCAGTTACCTCTGAGTTTCAGTGACTTCTTCACACACCTCCCACATTTCCCGAGAGCCCGCAAGTCCTTGAC
AAATCATAGCAATGGCAGGCCCTGGGGTGCTAAGGATACTTGATTCTTAGAGTCAAGTAGGGACCTGGACAAAGGCAGAAGTAAGGA
CTCCATGACGTGTCTCCCCTTCAGCACTAGTGTATCAGGATCCCTCTGAGGAGCTGGCAATAGCCAGTCACACACACACAGACATTA
GCCGGAGACAGAGGTACTGCCAGACCAGGGTCAGAGGCTGCCCCCATGTCTTGTCCTGCCTACTGTGGCAGCTGCACCTGGGCTGGA
AACAGAGATCAGGTTCCAGCCTTCATCTCCTTTCTCTTGCTGGCGGAAGCCAGTTTGGTCAGTTATCATTTGGGGTTTTTTGTTGTA
TACATGTGAAATTAAGGCAACAGCTTTCTAGCTCTGTTAACTTTGGCCTGCCTGCTTGTGCCAGGGCTCTATGACTAAATGGGCTGT
CCCCTGTCTTCTTCCATCCATTTGCTTAGAAAGCATTCACTGAGTAGCAGGGACGCATTGGTAAGGGGGGTGTGGCAAATAGGAACA
GCAATGACAAAGCCAAAAGTGCATCATGTTGGATGTTGTGCAATGCAGGGGTGGTGGGGAGGAGGTGTCCTAGGACAGGAAGCCGGG
CTTGGTAAACGCATCGAGGATGCAGGTGGGAACAGGGTAACCTGGAGTTTTACCAAGACCCTGCCGTGTGAGCCAAGGACTACTGTT
GTCTGGGAGCATCAGTAACAAGCAGGAAAGCTAGGAGATGGTACTAAACCAGGCAGTTTGGTCGCTGAGTAGATGTCAGAGGAGGAA
GGGGAGGTTTATGGCACAGGAGCTCACAGAGGCTCCATGGGTCTGCAGGGATAGTCCTTAGGCACTTGAAAGTGCTGGTGTTTGCTC
TGAAGGACCCAGGGCTGTCTGTCGTGTTCTTTGATAGGCCATTCCCTTGCTGCAGTGACACTATGTGGCCCAAGGTGTGGCTGTACA
AGCTGAAACAGAGAGTACAAATTAGCCAGCACCTCCATGGGCCTTGTGACAGGACAAGGAAGGCCTGAGTGCTCACATTCTGGAACT
TCCTTTAAGGCAAAGATTTGCTGGAGCCAAGGAAGGGGCAGTGGTCCACACAGAAGGCAGAGCGACCTGGCAAACTGCAGAGAAGCT
AGGCTTTTCCCGAAAAGCACTGATGCCTGCTTTCCCTCCACGTCTGTGCCACCGTCCTTGCCACTAGGGCAGAGCCCCAGGTGGGAG
GCTCTTCCGGAGGCAATCACATGCCTCTATCTGTGTCACGCACAGAAGGCTGACAGCTCTTCCACACTCTGGTTAGATAGGGCTGGA
GTGATTAGACCTGATTCCCTGAAGACAGGACAGGGACAGGATTTGTTCAAGGTCACAAAGCCATTAAGGTATGAAGGACAGTCTTTG
GGTGCTTCTCCAGTTCTGCTGAAACAAGTCTCACCCAGGAGACTGTCATGACAGAACAGCTAACAGTACTTAGACCCGTGCCAGGCT
CTGTCCTCATTCACTGAATAGGAACTCTGTGAGCCGGCATCTCCTTTTACTGGAAAACCGGGGGACAATGTACTGCGTGTACCGTGG
GGATGGACCAAGAATTTGTGCTCTGGTCCCAGAGCCTGCGTTTCACATTGACACCAAACAGCAGCACAGAGCCAGAGCAACAGCTGG
GTAATGAGGATGCTCTATGGTGTGGGCCAGAGCAACAGCTGGATAATGAGGATGCTCTATGGTGTGGGCCAGAGCAACAGCTGGAGA
GTGGAGGTGCTGTATGGTGTGGGAGTCAGGGGAAGAGCTCCAGCCACCAGAGGGTTTGAGCTGGGTCTGAAAGGATCAGTTAGAGGG
ACCAGTGAAGACCAAGGCAGACATGGTGGGATGGAGGGGCATGTGCACAAGGCAAGCTTGGGACACCAAGGAAGTCAGGGCAAGAGG
CTTAGAAAGCAAGGCAAAAGGGGGAAAGGAGGACAGCAAGCTACCGAAAATGCCAAGGGGCAGGGTGATGTGAGGAGAGCAGTGCAG
TGGGGTCCGAGGCTGCCATGGAGCCACATCCATGCCGCGAGTTCTTCACTGGAGACCTGAGCAGGGACTCCAGGCTCAGCGTGGTAT
TTGACTATGGGAATCTGAGCGTGAACTCCAAGCTGAGGCCATCATCCATTTGGAGGGAGTTATGCGGAAGGCCTGGCTTGAGCTGTT
CGACATTCCAACAAACAAACCAGCCCCAGGACAAATGGCTGGAGCCATCATCCTTGGGATCCATATGGAAACACTCCTGAAGGAAGT
CTACTTTACACAAGTGTCCAAACTCATTTGGAGACAAAGGGAAGAGGCGTATGCTCTTCACGTGGGCCTTGCTCCCGTCACCCTGTG
CTGGAACGGGTCACAGCCAGTGTCAAGAAGCTGAGTTCTTATTAATAGTGAGAACCAAGTCCTAGCTCAAGGAATCTGGCAGCCAGT
TTGAGAAAAGTCAATGACTGAAGACAATGGCTGGAATCCCACCACACTCTACTGGGCCACTCCAGTTGGTGTTTCTTTCAGGATGGC
GCAGGAGCGGCAGGAAGAGCCATCTTCGTGCCACAGACGTTTAGTGAGCACCTACTACATGATAGGCATGCAGCTGGTACCCAGAAA
AGGCTACAAAGAGAACTCTTATGTAACTGTAGGGTTAATAGGCCAATGGAACAGTGCGAAGAACTCAGAAATAGAACCACTTCCACA
TGGAGGAAGCACGGAGAGTCAGGGGGAGGAAACCAGTTTCAGAGGAACTGCAGAAAGTCTCATGATCTCAGGAAGAGAGGGGTTTCC
TAAACATCACACAAAGCGCTAAGCTTAAATGACAATGGAGGAACACAACTTCAGTGAAGTCTAAAATGTCCATCAAAGACACTTTAA
GGCTGGCAAGGTGGCTCATCAGGCACAGGCGCTTGTCATCAAACCCAACGATCTGAGTTCCATCTCTGATCTACTTGATAAAAGGAG
GAAATCTTCTACAAGTTTTTCTCTGACTATCATATCATATCATATCACATCATATAATATGAGCCCCCCCATAAAAAAAAACAAATG
TGATTATGATCTCCAAGGGTCAGGCATTCCAAGGCATCTTCAGTGGGTACCTCTGGCTCAATATATCTCATGAAGTTACGACCTTTC
TCTCTTCCAGTGTTTAGGGGTCCAAACTCATCCCTGTGCTTGGTGGCAGGGGGTGGACCCTTACCATGGGGGCCTCACCATTGGTTT
GCTTCAAGACACGGCAATGAGTTCTCTCAGGAAGAGCAATGCCAGAGAGGGTAGGGGAGAGATTACCAAGACAGAAACTACAGCCCC
GCCGCAGTCTAACTTCTGAGCTCTGTGTCAGTCTAACTTCTGAGGAGAGCTTCAGTTGTCTTTGCTACATTGTATGCATTAGAAGCG
AATTAGTAGTTCATTCCATATTTAGAGAACAGGGCTTAGAAGCGAATTACTAGTTCATTCCATATTCAGAGAACAGGGCTGGTTCAA
AGGTGTGTGACTACCTCCGGATGGAGGGACGACTAGCAACCATTCCAAAAGCTACAGAGTACACTGTGAAGACGAAGCTCACGGTGA
AGAAAAGGAAGAGAGAGAAACATAAAATCCAGGGTAATGATCACCCTGGGCAGTGGTGATGGAAGACTGTGGGAATAGCTGGTGGGG
GTGAGGGCCTGGGGTTGGACCCCTTTCTAGATGCTTATTATTTTCTTTTTATGTTTTATGTATTGGTTACTTTTAAAAAAATTATCC
CTGCAACAAAATGTCCAGCAAAAGCAACTTAAAGAGGGAAGGCTTTATTCTGGCTTGCAGTTTGGGAATGTAGTCCACTATGACCAG
GAAAACATAGAGATCAGGTATGAGACCACCATCACATTGTATCCTCAGGCAGGATGCAGAGACACTGTGCTGGCACTCAGCTCACTT
CCACTGGAGCCCAGCTGGTGAGGCAGCACCCTCATGTTCAGACCATGTTCTCATGGTCTTCCTTGTCAATTAAACATCTCAGAACTC
ATAGATACACACAGGGGTGGCTCCTTGCGATTTTAAACCCTGTTAAGTTTAAGCATTTTACATCATAGCAATTGTTATATGTATTTT
TTTCTTCTCCCTGAATTTGTTGTTGTTGCTGTTGTTTGCTTGTTTTTGAGATGAGGTCTCTATCCCAGACCTCATCTTGAACTTGCT
AAGTAGCTCATCTCTTGATCCTCCTGCTCTGACCCTTGAGGGCCGGGAACCCTAGGTGTGTGCCACACTTCAGTTATATCAGCGCTG
GTTTTAAGGCCATGGCTTTGTACATGTTAGGTGTCTTGGTTACTGTTCTATTGCTGTGAAGAGACACCATGACCAAGGCAACTTATA
AAAGATACTGTTTAATGGGGGCTTGCTTACCGTTTCAGAAGACTAGTCCATGCCTCTCACTGCAGGGAGTGTGGCAGCAGGCAGGTA
GCTATGGTACTGGAGGAATAACAGACAGCTTAAATCTTGTCTGAAAGATGGGGCCGAGAGAGGATGACTGTGCCGGATGTGGGCTTT
TGAAATCTTAAAGCCCACCCCCCAGTACCACTCCTTCTCCAGTGGGGCCATACTTCCTAATCCTTCCCCCCAAACAGTTCTAGCAGC
TGAGAACCAAGCACTCTGATCTATGAGCCTATGGAGTCCATTCTCATTGAAACCATAACACTAAGCAAGCACTCTACCAACTGAGCT
ATGTCCCCAGCCCTTTGGTTTGCTTTTATGTTTGATATGCATGGCTTCAAAAACCTTTAACATTTACTGATTTATCTGATTATTTAT
TATTTGATTTCTATTTTAGGGGTTTTTTTTGAGATGATATCTTGCTGTGTAGCCTCTGAACTCACTACAATCCTTCTGCCTCAGACC
CTTGTGTGCAGAGATTGCAGATATGTGCCTCAGATCTTCCTGCAGGCTTCCTGCAGAGACTCTAATGTCTGTGCAGCCCTAGCTCTC
CCTGCTTATCCGTTTCCGGAGGCATCTGACTCCCAAGCTTCTGCTGACGAACTCAGCACTTTCCTCCAGGTCCCTAAAAGTCATGCT
TGGAGTGTGCGCTTCTGGTGGCTTTGGTTTCAAGAACTGTCTGTTCACTAACCGTTGAAAGGTGAAATCTCGTTCCTTGCTTTCCCC
TCATCCCCTCAGCTCTTCCTGTTCCCAGTTGTTTTTTTAAAATAATAACTCTATATTATTTTGGCTAAATCAAAAATCAGTGTTTAC
GTTACTTAATCAAGACTAATTTGACTCTATTCATGGTCGGATCCCATGGTAAAATCCAATCATTTTCCCTCTCTTGCAGCCCTTTCA
TCTTCCCTAGAGTTGCTAATTATCACTTTTCTTCATTTGCTTAGGTTTCTGTGTACTTATCACTTGTTAACTCCAACCTTGCCGTTT
GTCAGAAGTTCAGGCCTCTTATACCTTTTTAAAAAATTGTCTTCCTAAAACTGGCCCTAGAGCCTCCAGCCTTCAGACTGGTGCCCT
GGTCTTTTCTCCTCTGCAGGACAGATTTCTCTAGCCCTGGTCTTCCTTCTCCTGGCCTTTGGAGAGCCCAACTCCACTGGGCTGTCT
AGTTTAATGTCAACTTGACACAAGCTAAAGTTATCTGAGAGGAGGGAATCTTAATTAAGAAAACACCTCCATAAGATCTGGCTGTAG
GGCATTTTCTTAATTTGTGATTGATGGGAAGGGCCCAGCCTGTGGGTGTTTCTGCCTCAGGGCTGGTGGTCCTGGGTTCTGTAAGAG
AGCAGGCTGAGCAAGCCATGATGAGCAAAGCAGTCAACGGCACTCCTCCATGGCCTCTGCATCAGCTCCTGCCCCCAGGTTCCTGCC
TTTGTGCATTTCTGCCCTCACTGCTTTTGACGCTGACTTGTTATATGAAACTGTGAGTGAAATGAGCCCTTTCGCTCCCAAGTGCTT
TGGGTCATGGTGTTTCATCACAGCAATAGTGACCCTAACTAAGACACTCACATGGCACCCTGGGGAAAACAAAGCTTCCGGACTGGT
GGTTCCCAACTTTCCTAATGCTTCCACCCTTCACTCCAGGTCCTCATATTGTGGGGACCCCCAACCATAAAACTATTTTGCTGTTAC
TTCATAATTGTAATTTTGCTACTGTTATGAGCTGTGATATAAATATCCCATATGCAAGATATTTAATATGAGATCCCCCATAAGGGT
CTCATACCCACAGGTTGAGAGCTGCTGTTCTAGAGCCTTGTGAATAGGGAAATACCCTTTACTCTGTCTCGTTCAGACTCAACTAAT
GCTTCGGCGCTGTCTTTGTTACATTTCCATTGCAGTAAGACACCATGAGGCAGGGAACTTACAGAAATGAGAGTTTATTGGGACTTA
CAGTTTCAGAAGATCCATAATCATATGGCAGGAAGAAGAGCAGCAGGCAGGCAGGCAGGCAGGCAGGCAGGCAGGCAGAGCACTGGA
GCAATGGTTGAGAGTGCATGCCTTGAGACACAATCACAGAACAGGGAGTGCTAACTGGAAAAGAACCTTTGAAACCTTAAACCTCAT
CCCCAATGACACACCTCCAACAAGGCCATACTTCTTCTCTTAGTTGGGGTTACTATCACTGTGATGAAACGCCATGACCAAAGCAAC
TTGGGAAAGAAAGGGTTTATTTAGTTTACACTTCCACATCACTGTTCATCACTGAAGGAAGCCAGAAGAGGAACTCAAACAGGACAG
GATCTGGGAGGCAGGAGCTGATGCAGAGGCTATGGAGGAGTGCTGCTTACTGGCTTGCTCTACATGGCTTGCTCAGTCTGCTTTCTT
ACTGAGCTAGGACCACCAGCCTGGGATGGCCACACCCACAATGGGCTGGCCCCTTCCACATAAATCACTAATTATGAAGATGTCCCA
CAGGCTTGATTACAGTCCAATCCCATGGAAGCATTTTCTCAATTGAAGCTTCCTCCCTCCTATCTGATGACTCCACCTTGCATGCAA
GTTGACAGAAAAACCATCCAGGACACTTCCTAATCCTTCCCAAACAGTTCCACCAGCTTCGTACAAGTCTTCAAATATCTGAGCCCA
TGAAGGAACACTCTCACTCAAACCATCACAGATGGTCTCTCCTGACCAGAATTCTGATTTGGAAATTATTTTCCTTTAGACTTCCTC
CTAGACTTTATTTTATGTGTATGAATCTTTTGCCTGCATGGATAGACATGCACCATGTGATGCCTGTGGAGACCAGAAGAGGGCCAT
CGGATCCCCAGGAGCTGGAGTTACAAACGGCTGTGAGCCACCATGTGGATTTTAGGAACTGAACCTGGGTTCTGTGCAAGAGCAACA
AGTGTGTCTAACCACTGAACCATCCCTCCAGCCTTGTTTTTCCTTTAGACTTTTGACAGATGTATCCCATGCCTGTCTAAGTTCCAT
GGTTTCTGGGGGAAGTCAAAAGTCATAATCAGTGATGTCTCTATTTGACTTTCACCCCCTCCTCTTCTACTATCTCCTGCTCATGGA
ACCCTGCAGCTCTCCTCCTCCTCAGTGGCACTGAAAACCTACATAGTCATGTGCCTCGCTGTGAGCCAGTCGCTGTCCATTGTGCGG
GGGACTTTGGTAGAAGCTCTCCGGATGCCTCTCCAGCCCCGCCCCACCAGTTCTTTCTCTCTGGAACTGATACTTGTATCTGTAGAC
AAACCTACCAGTTTCCTTGTATGTCACGTTCCTTTTTGTTCTTTCTGTTTTCTGCATTTGGGAGTCTGCTTGATTTCTCTTCCCGCT
CCGTCCAACTGACTTTCCCCGGCAGAGCGCCCCTTCTTTTTATTTTGTTGAAAATAGACTCTTCTGTCACACAGTACATCCTGGCTA
CAGGTTCCCCTCCCTCTGCTCCTGCTTCACAGGTGCGACATCTTATTTTATCTCCCTTGTGATGATAATTATAGATTCCCTCCTCAC
TCCCACGTCTGACTAGCCCTGCCCAATGGTTCTTTTCTGTGAATGATCTGTTCGATCGATCCCGATTCCGTTTCCTTTGNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNAGCCATGGTCCACCCTGAGGGGAAAAATTAACAA
TTATAATAGTATTCTTGAATTGAAAGAAAAACTTCCATCTTTTCTTCCTCAGCCCTTAGAGAAAAACATGAAACTACTTCCTCTCCA
AAATTCTTTATTGCAAAAAATAGCAGGAGATTAGTACGTGGATGTGTGACATGTGTAAACCACTTAAAGCTCTGACTAGGCTTCGAG
CTGCTCTCCCTCCCACAGCCCTGAATCACCAGCTGACTGCTCCTGCATCACTTCCTGTTGGTGAGTTGCAAGTGGGTCTCATCTCCA
TCACGGCTCTCCTGCCACAGCAGAGCAGTGGCTCCCAGCAGCAGAGCCAGGTCAATCAATGTCAGCTTCTCTGTCACCCCACAAGAG
GAGGGGGCAGTGGTGGGCACCTAGTGAATGGGTGGCTTTCTCAGGATATATCCTCTCCTTGTCACACAGCAAAGGCTGTTTTTGTCT
ATATATGTATATGTTCAAGGAAAAATTTTATTTTGTATTTTAGGCATATATATAGATAAATAGATAGATAGATAGATAGATAGATAG
ATAGATAGATAGAGAATGAAAAAAAGAAACCCACAAGAAAAATTTTTAAGAATCATTAAAAAAGCAGTTTTATATCATCTTTAAATG
CTTGCTTGCTTTTTTTTTTTTTTTTTTTTTTTTTTTTTTTTTTTTTGTTTGTTTTTTGAAACAGGGTTTCTCTGTATAGCTCTGGCT
GTCCTGGAACTCACTTTGTAGAGCAGACTGACCTCAAACTCAGAAATCTGCCTGCCTCTGCCTCCTGAGTGCTAGGACTAAAGGTGT
GCACCACCACACCCGGCTTGCTTGCTTGCTTTTTGTTTTGTTTTGTTTTGTTTTGTTTTGTTTTGTTTTGTTTTGTTTTGTTTTTGA
GATAAAGTCTCTGTATCTGGCCCTGGCTGTCCTGCAAGTCACTATGTAGACCAGTCTAGCCTCCAACTCACAGAGACCTGTCTGCCT
CTGCATTCTAAGTGCTAAGATTAAAGCTGTGAACCATACCTAGCTTGCCCTGTATGTAATTCCCCCTTTGATCTCTAATCTCTGCAC
CTCAGAGTTCACAACCGTGAAAATGGTTGGTGTGATGCTGAGGCCAGGCACCCGCTCAAGAACACTAATGTAAGCTGCAAGGTTTGC
CAGGAAAGACCTGTGAGGTGCTGCAGGCACACGAAGCAACAACATGCACAGTACTGCACTGCCGCATACTAATCCCAGTCAGCGGGC
TGACACTCTACGGTGCCGGTGCCGGTGCCGTGCTGCTGCTCTGCGTGCACAGCAAAGGTGCTTCCTGCTGGGGCCCGACCTCTGTAT
GCTGTTGTCACCTCGGCACGCTCAGATGGCAAGCTTCTTGTCAGTGGCCTTGGAGCAGGCTCTTCTGTTCCCAGGTCAAGCACTTCA
GCCTAAACTTCAAGGCAGTCCTGTGAATGCCCTGCTATGCCTTTCCTCTGGTGTCATAAATCCTACCTTTTCTGTTTCTGCCCTTCC
CCGTACAGCCACTGCTGATATGTGCTAAATACTTACTGTGGGCTACAGACTTACACTGCTTGCTTGCTTGCTTGTTTGTTTTAATCT
TCCCATTAGCAGATAGGAAAACTGAGTCACAGAGGGATTTACATTACCTGTCCACAGCCACGGAGCACAGTGCAGCTGGCACGGACC
ACACCCAGATGTTGCTCATGCCTCCTGACTGAATCCCAGTATTCTTAGACCGGACCCAAGCTTCCCCTCCAGAAAATCCTCTTCCTG
ACTAGGCGCACTGGCTGTGTGCTACCGGCTGTGCCCCTGCAACGTTTGCTTCCGTGCTGCCGACTCATTGTGCAGGCTCTGTCCGTC
TGTCTGTCCTTCCCACTGCCACTGAGCCTTGCTAGGCCGCCAATCGCAGAGTCTGGTTTTGTAGTCTAGAGCCTGGGAGTCAGAGCT
TGTTGTCTCCTCCCAACATCCAGGAAAGAGCTACAGACCAAGGACAGGCAAGTGGGACAGTGGACAGATACCTGTCAGAGCAGGGCT
AAGGGTGGGAGAGTGGAACAGGATCTGGCTTGGTCCTGGAGGAAACAGTGGGGTGCTAAGCCTGAACCTCTCCAGGATGGCCATGGG
AACAGAACACGCCAGCCTGGGCACATGAACCTAGAAGAAGAAGCCAGCCAGGCTCCTAGCATGGCTGAGAATTGGATGACTGGGCGA
TTAAAACTATCTTCAACTGTCCCCAGAAGCTCCAGGCACCCAGTCCCTTGTACAGGCAGCAGTAAAAGCCACTGCCATGGTGATGAT
TTGGAACTGGATTTGAGGGCAGTAGGCAGTGGTGGCCATCTTGCCCCAGTTCCGCACTCTGCAGACCTAGCTTCCTTGCTGCTGTGC
CTCATCTTTTCAAGCATCCTGCTTGTTCTTCCTCTACCAGGGAACCCAGAGTTGCCTCCAAGCCCTCTAATGCCAAATGCAAACACC
TGCCCATAGAACCCCAGGCCCTCCAGCCTCTTCTGAGCACTGGTCTCCCAAGCTGCGTGTCCACTGCAGAAGCCCAGGTGACAGGTG
ACCGCCTCCCGTGGACACTTTCTCTCTGGGACACGTGCCAGTCTCTTCTGTTTCTCTGCTGTCCAGTGGAGCAGCAGAAGTCATGGG
CTTTGCATCAGTTGCTTCCTATGAATCACTCAGCATCTCTGTGCGTCTAGCCGCTTCCCCGTCAGGACTTGAAAGGCTGCCGTAAGA
AATAGAGTGTTTATGTTCAGAGCAGCGCCAGTAGATATAACCAGACAGCAGGTGCTCTGTAGATAAAATCCTTTGCACTGCCTTGGT
GTTCCTCAACTAGGTGCTTGTCAGTGACTAGCAGCTTTTCTGCTTCCTTCAGGGGCATCTCCAGATACACTTGTGCTTGCAGATAGC
CCTGCATGCCTCACTAGGAAGGTGGGTCTGGGGAAGCAGTGCTGGGTGATCACTCATGGGGTATCAGTCTCGGTGCATGGAGATTGC
AGCTCAGTATAGCTCGGGCTCAGTCCTTACGTATGCCATGTGTGTTAACAAGTGTCAGTCTCCTTGGTTACGACATTCCCAGAGAGC
TGCCCCCTTTACTCAAACAGGCTGGCTTGATCTCCCTGCCCCTGGCTCTCTTCCCCACTGTTTATCAGAAGTAGGATTGCACTGCAT
GCCACTCACGCTGGCCCAGTAAAATGACCCTTTGAGAAAGGACCTTCCTTCCTTGCCATCCCCACTCCCCTGCATGCTGAGTTTGGG
TTCCCAAGACCTCCTGGAGAACTGCAGCCCTGCCCCCACCTCAGTCCACGGTGCTACTGTCACAGAGCACCACAGACTGGGTAACGT
ACGAAGATGAAAAGGCTAGTTCCCATGGTTTTGGATACTAGGATGCCTTTTATCAAGGCATTGTCGAAACACCAATAAGTTTAGCGT
CTGGGAAGGCACCTGGTTGACACATCCTCCAGAGGGAAGGAAACTATGTCCTCATGTCTAGAAGGCTCAAGAGCAAAGCAGTAGACT
GGTACACAAAAACCCTTTTAAGTCAGGTGAAGGGACACGGGCCTGTAATCCTGTCACTTGTGAGATGGAGGAAGAAGGTCAGAAGTT
CAAGGCTAACAGCACATGAGAATGTCTTTTTTTTTTTTTTAAGGAATAAGTAAATAATAAAATAAAATAAAATAAAATAAAACAAAA
CGTTCCCAATTCATTCATGAGAGGATACTTCACAGCCTAATCATCTCTAAATGGCCATCTCCTATCATCTTGGGGACATCGGCTTTT
GAGGAGGGAAGACATTTGAGCAATGCCAGGCACCTTCAAGCTGAGTGTGGAGGTTCTTGCCTTTGATTCCAGCACTCAAGGAGGCAG
AAGCAAGGGGATCTCTGTGAGTTCAAGGTCAGCCTGATCTCCATAGCAAATTCCAGGCCAGCCAGAGCTACAGAGTGAGATCCATTC
CAGGAAAAAAAAAAAAAAAAAAAAAGGAGGCACCTGCCAGTGTCTCCTACAGAGCCCTCTGCAAATGTCTGTATATCCTCAGAGCAG
AGCCCCAGGGCCAGGTTCTCCCGAGGAAAGGAAGGAAATACAGAGCAAATGGCCTCCAGGCAGAGGAAATGAGATGATAGTTCTAGC
TGTGTTCTGCCACTAATGTTTGTCTGGGTCAGAAAGAGGCTGAGCCTTTGTGAACACTTCCGCGTTCCCCCACCTGTGAGGTCGGTT
TCAAGAGAGAGACCCAAGCTACAATGGCTTCCTTCTACATACACAGGAGATACCAGTCCTGAGAGAGAGAGAGAGAGAGAGAGAGAG
AGAGAGAGAGAGAGACAGAGAGAGAGATTGATCTTGTCCAGGAGTCTGTGATTTCAGATTAATCATATAGACGGTCGATTTAGAAAG
AAAATTAGAATATTATGCATGCATGACCTCAGCTGAAGCTGTCCTACCAACAGCAAAAGGAAGTCTACAACGCATCACAGATCGTGA
CAAAACATTTTTGCCTTTGGGACTCAGCGACTGCAGGCAGGCAGGACCTGGATGGGCTCTGATGCAGCCAGCACCTTCGGGAAGGCC
TAAAGAAAGGTGTCTCACTGACACCACTGTGTGTTCCTCAGTACCCCACCCCACCCCCCTAGACTGTTCCACAGTCTCTGTTCCCAG
GAATAGGAGTTTCACAGACTAACTCCCGTCTTCCCTCGGCAGGTACCATGATCAGCAGGATGTAACTAGTAACTTTCTGGGTGCCAT
GTGGCTCATCTCCATCACGTTCCTTTCCATTGGCTATGGGGACATGGTGCCCCACACATACTGTGGGAAAGGTGTCTGTCTTCTCAC
CGGCATCATGGTGAGTACCGCCTCTGCCCATGGTCCTGCCCTCCCAGAGGGGCCTTCCTGGGGAGGGGGAGGGGGAGGGGTGCTGGT
CACTAGGCACTCATGCCTCCCCTGTGCAAGTCGAGAGGCAAGTGTGAGTTGTGCAAATCTAGTTTCCAGAGCCCACCAGCTGGAGTT
GGCTGTCGCTCTGACCCTCTGCTCACCACTGGGGAACCTCAGGCTTATCATCTGCCATGCTCCTTTCTGTGTCCTGGATTCCATAGA
CCTCAGCACCAGTTCTGATCAGGTTTAGCTGGAGCTCAGAAATAGACCATTGAGCTGGCCTGAAGGGCCCTGGGTCCTGAATGGGCT
TGGCTTGCTGGCTTGAGTTCTGGTTTGTCCACATGGCTCTGGGACAACTCTTGGCTTTCCCAGGCTTTGTCTTCCACGCTTGCACAT
TGGATCGCTGGCTCTGAGTGACAAATGCTTTCCAGCATGGGTATCTGGTAAGACCAACAGGTCTGCAGGCACCCAGCACCAAGGTCT
GCTTCTTCCCACCCTCAGCCAAATGCCTCAGGCTCCAGGGAGCTGGGACACACGTGTTCCTACCTTTCGCTGGGCCGTGTGCGGCAT
ATGGGAGGGATGTGGTGTGCAAGTAAGACACAGGGCCCAACTTTTCACTATCCCTTCACTTTGGCCCCTGGGCCAGTCACTCAGCGG
TCACCTGTGGAGCCTACAATCTTTGGGTATAGCTAGATACTCAACTGGCCTCCTCACTGACTTGTGGGCCCCAAGATATAACTGATG
TGAACATTTTGGGTAAAGAGTGAGTACTGTATTCAAGCTAGCCCCACATTTGAAGCAATCAGGATTGGGGGATATTTTAGGGGTAGG
ATAGGGAGTGTCTACTATGATGTCCTATGTGGAGTCTTAGAATCAATGACAGACTTCCTTTTTAGCCCAGGGAGACCTGTTTCTTAG
AAGAGAAAAAAAAATCCTGGAGAGAGGGGCACACAGCTCTGCTGGGAGCCATTGAAGCTGATAATGGGGTGACAGTACAGATAGGAT
GTTTGAGGCCTGGTTCTAGGTCTCTCTCCACAGTCTGACCCCCAGATCTCTCAGTGCTGTCTTGCTCTGCACCCCGCCCAGCTGCTG
TGAAACCCCTTTCAGGGCTCCGTTCTCACTGCCCCCAAGAGATAGGACAGCCTTCCTGTGAGCCAGTGACTGGTGTCTACCGAAGGA
GCCAGTCGGCTGCTAGGTATAGAGGAGACCCCAGGGCGGTGGCTGGCTGACCACAGCCTCCTGTTCTTTCCAGCTCCCAAGCAGAGC
GGGCCCATGTCAGCAAGCAGCCTTGAAACAGCCGGTAGTGACAAGGGCATAGTGCCAGGCTGGGATAAATCACAGTAAGTGCAAGGC
TTTAGAACAAACACCTCCCATCGTTCAGTAGGAGTCTGTCTGGCACAGCGCCAACAGTCACCAGGAAACCAGGCAGAGTCTTCCTAC
CAGAAAGGAGTTAAATGCCACCTGAAGACTGTAGCTTTCCTTCTGAGACTCTTGGAGTGAGCTTCAGGCCCCACCCTAGTTCTCAGG
CAGGGTACTAGGAGCCTCCTTGCCAAGAGGTGCAAGAGCCTCTGTTATTTTTAAATGCCAGCCATTCTTCCTCTCAGAATGGAGCTG
TGCCCTATCCAAGAAGCTGCCTGGGCCCACTGTTTCCCTGGCTCCCCGGTTTCACATCTCCTGGCTTTGGCTCTAAAGCATTGGTAA
AAAGACAGGAGGCCCTGGTCTGAGACACTCCTTATCCGTAGCCAGTGGTCTGCGTTGGCCTCCCCAGCACCCTCTATGGCTGCCTGG
GATCCACGGTGTTGCTTGTGGTTTGTGGTGAGATTCAGGTGGAAAGAGCCACCAGGCAAGAGGCCACACTGCCCTCCTTAAATTCCA
ACACAGGCTTTTCCTGGGAGCGCTTGCCTAAGGCCTAGCTCCCCACAAAGGCTCCAGCATGGGACGGTCCCTTTGTTCTTTGTCAAT
GTCTTCACCCATATTAAATGCCACTCACTCTGGAAGCCTGGTGGCTTTTTGTTTTTCTTTTTCTCTTTCCTATTCAACAGGACTCCA
GCTGAGTCATTCCTTTCTAGGGGCTCACATTGCTAAGCCCACCCCCAACATGATGTATATCTCCTGATTATTATTCCAAGGGCCTTG
ACTAAAGGTAGGGCTTCCTGTTTGGTTGCCATGGACACCACCTAGGCCAAGGCAAGGGCTGCACAGGAAGGGAAAGGAAGGATTTTG
TGTGCCTGCTACAAATGGCCTTAATCATTGTTTTCCTTTGGCATCCAGCAACTGCATAATTATCCACCATTAGGCAGGGCATATAGC
TGTCTGGACAACTTGACACCGTTGGCTTAGGCCTGGAGCACTTGAGAGTTCACTCATTAATGCAAATCTACCTTGAGCCCCAGCACC
TTCCCATGAGGAATTTTCCAGGATACTTTACAAGGTAGTTACTATTCACCACCCAGAAGGAAAATGAAGCTTTTGGCTTAGCTTCTG
TGGGTATCTGGGAGAGCCTCTGGGCCCCCCAGAGACTTTCTGGTGGTAACTTCATGTCAGTGGCTCTCTCTGTGTCCCAGATTTGGA
TGCTCTCTGTCACCCTGATGCGGGTTGTAGACAAACAGGCCTTGTAGCCATCCATCCAGTGGAACAGTGAGCTTTCCCCAACCGGGT
CTCGAGAGATGCCTCTTGAGCTGATGAAGAAGAGGTGACACAAACCGAGTGACCTTGGGGGCAGCACAGATGGTCTCAGGAAGACAT
CTGCTTAGTGTCCTGTCCCATGCAGCTCTCCTCAACCGCTGGGGTTTCAGAGCTGCTGATGATATTTTGGCTGTCCCACAGAATCTA
TTGCAGCCCCGAGGCGACCGTAAATGTTCCTGGCTGCCTTCTTCCATGTGTCACGTTCATTTATGTTTTATCTCCTTTTATGCCTCT
TTAGTTAAGAACACCTCATACTTTTCCCCCTCGGGATACTTGTTTTTCCAACATCTAAGATTTTCCAGCTGGACATTTTTCTTGGAA
ACGGGAAGTGGGAGGCATGGTGTGGAGCGGTTGGTGTCCAGATTTGGGTGACGACTTAGACATGTTCACCCCTTTAGAAACCCCTTC
TCTGGGCAGTCTGTAGCCTCTGCTTACCTGTGTACCTTGGGCAGTCTGACTTTGTTGGAGGCAGGTCCTGCCCCGCTCTTTGCAAAG
AAGACTTGGGTGGAATGGACTGAAGATTCCCATCAATGTCAAGGGAGTCAGGTCTTAGCATCTAAAGTCAAGAGGCTGAGTCACAGA
TTTGCTCCCCATTTTAACCCCACAGAAGTCACTCCAGCAGCTGTCATCTTTCCTATCCCATCACCCCCCCTCCTCATGCCCCTCTGT
CCCCCTCTGTCTCCCTCTGTCCACCGGCCACCATCACTGCGTTTCCACACAAGGACAGCGTGAGCTCACATGCTGCACTTAAGCAGG
TCCCTGAGAGTCCATCCATTAGAGCAAGCTGCACAACGGCTGAATCTCACTAATGCGCCCGGGTGGCTAATACAACCCTGCGGTCAC
TCTCAGATACAAGACATTTCCAAGGGAACACAGAGTGGTCACCATCAAACCCATAGGGATAGACAGGCTGGCCATTGGGAAGGGGGC
CTCATGGCACTTCCTGGGTGTCCTCTAAAGACCTCAGATGGATCATTCGCTCTAACCCTGGCTCTTCACTCTTCCTTCTCACACCCA
ACTGTTGTTTGGTTGTTTCGTTGTTTCTTATCCCTGGCTTTCTCCTTCTGTTTTCTGATTAGTGCTGATTAGAATATGGCCAAGTTG
TTTTCTATCTCATTCTCCATTTCCCCTGCTCCCCCATCTTCTCAGTCATCCTCGAGAGGACCAGCAGCCTGGGCTGTAAACACCTCA
CACCATTTTCTGGGGGTTGTTGCTGCTTGATACCACATTCACAGAGTTGCCTCTTATACACATTAATAGCTCTGGCATCATCATCCG
TGATCTCCCTAATTACATCCAGTCATTGAATTATCTAAAAGAAAGCAAAGGGTGAAGGCTGGCAAGACGGCACATTGGCTAACGGCA
TACTGCCAAGCCGGATACCCTGAGCTCAATCCCCAGGACCCATAGGTACAAGGAAAGAACTGGCTTCTCTTCTGTTGTTCTCTGACC
CCCATGTTTGCACCCGTATACATACACATGCACACAGATAATTAAATCTAACAAATTTCTCATTAAGATTGTGCATACCCACTGAAG
TGGGAATGGCGTGAAGAAGGTAGCATCCTCAGAAGGGTATCTCTGAGGGAATGTTTACATTTTTAGGCTGAATAGAAACCGGGCTCT
GGGATTTAGTCTACTTTCTTGCCTCCATGCTGCCCCCTCAACCCCCAAAGTATCCTTCTCATGTAAGATGGGTATTTCCTTAGTTAA
GCACTGCCCTTCGCATTGGCAGTCCACTGTAGTCACAGCGTCAGCCAGCTAAGCTGTGGGCCAGGGCTGGAGCTGCAGGGCTCCCCT
CTAGACCCCAGCAATTGGGGCATGACCAATTGTTTTGATGATGTTCCCAAGATCCATATAGCAGAAGGGTGACTCGCTCTTCAGATG
TCACACCCGGCTTTCCGGGGTGATTTAAGTGGAATTATTTCTGGACCCAGGAGAGCCTGGGGAGAACCCTCTCCCTCTGATGTCCAT
TTTTAATGCTAGGACACATCCTTCTTTCTGGATGTTCTATACAAAGGAGATGGATGGCGCTGGCTTTGCTTGGGGGTGGGGTATCCT
CCCTCCAGGACAGTTTCTTTTAAGCTATTTTTGCTTTTGACAGGCTGGTTAAATTGATTACAGCTATAGACTGAGCTGTCCAATTGC
CCATATCCTCTAAAAATTGGGCACCAAGTCTCCAGCAAGTCCCAGATGCCAAGAAAATGCAGCCATCCCCAAATGGACAGGGGAAAT
GAGGCCAGAAAAGAAAAGTTCACAGACTGGGCTCTGCCCTCGGATTAGGTGGCCGAATGGGCAGAGCGTCCTTCTGCTGTGTTAATG
GGATCCGTGTGGAAAGCTGGGGAGAGGCAGTGCCACTTTCCACATCAGCATCCCACACAGGATGGCCTTCATCACAGAGAAGGGGGT
GTGTGAAATACATTTGCCCCAAGTTGGCTATAGATTGTAGCATTTCTGAGAAATATTCTCCTCATTTCTCACATGTAGTATAAAACC
CAGGAAGCCAAAACTGTTGCTGGTAGATTCCTTCATCCCAGCATCTCAAACTGCTAATCTGGGCTCCATGCTGGGCTTTGTGGCATG
GGGACTGGTTGCTAGAATTCCTAAGTCCTCTACCTTTCTCCAAAGTTGGTGATCAGGATGGCACCATACTCAGAGAGCAGTGGTCTG
TGAGGAGAGGCCAGGGATGGCTTCCATGGGCCACTAGAGAACCAGGTGAAGGGATTCTGTCTCCCTCCCTCAGGACTCGCAGGCCAC
CAAGCAGCACCAAGGAGTGTTTCTAAACATAGGCGTACACTGCTTTCTTGACCTTGCTCCCTGCTCCGACAATGACAGTTGGGTGGA
TAGCTGAAGGACTAGGCAGGAAAGGCAGCCTGCTTTTCTGTGGCTCAGCTCAGGCCTGCTGCTTTGCTCCTCCTCTGTGTTCCCCTG
AGGGAGTTGGGAGATGGGAGCTACCTGAGCAGACTGAGAACATGATTCTGGAAGGGCTGAGAGTGTTCTACTGGGTCACTTAAATCC
TGGCTGTCGGCAGAAGTGTTGTCCCCTTTAAAATGTACTTGAGGCTCCTTCTGAAAGCCTGTGCTGAGTCACAGCCTAGAGCCTCAA
GAAGAAAGACCCACACACAGGGCAGGCACAGCAGTCAGTGCCTATCCAAGTATCCCCCTGGGAGATACTCATCCCAAAGATGGCCAT
ATACCCAAAGATGGCCATATTCATCTTTGGGGAAAAGTTATCTGCTCTTCATTTGGCTGTTCTCGGGACTCTCTCAACTGCCCGGTG
TGGGCTACACACTGCAGTCTGCACATGGGTTTTCCCCCTGGGCCTGTGGATGTGAGCTTGGGACGAATGAGACAAAGCAGACGTGGC
CTCCAGGTGCTGGACTGTCTGTCACTTGCCACACTGCAAGGGGCCTGATTACCCTAGGGTTTCCCTCCGGGAAGGCTCATTCTAGCC
ACACAGCACTGAGAACTTGAAGAATCAACTCCCACAGGTCAGCCAGGGTCACAAGGCAAGAAGCATTGCAGACCCCCTCTTTCATGG
GGCTATTGGGGCGCAAGCAGGTTTTGACAAGCTTTATGGCATGTCTTGACCAATTAGAGACTCAAAGTCTTATACCATTCCCTTGAA
ATGCCAACACTAAGCCTGGGGCACACCAAGTCTTGTGTTTTACCTCCTAACTGTGTGTTTTCTCTCCTGGGACCCTCTGACGTCCTC
TGGCAGGGTGCAGGCTGCACTGCCCTTGTGGTAGCTGTGGTTGCCCGAAAGCTCGAACTCACCAAAGCAGAGAAGCATGTGCACAAC
TTCATGATGGACACTCAGCTCACCAAACGGGTAAGCCACCTCACACCCATGCCTTCATGCAGGTCCACAGCCGAAGTGGGAGCTGAT
CAGAAAAAGATCAGACACCGGGTGTGGCTTAGCAATGCTCCTTTGAGTGCAGGTTAGGAGCATGCAGCTCAGGCGCACAGCCCCTAC
TGATGCACTACCCAATGCCCCTACTGAGTGCATGAATCTCAACAAACCAGGTGAGTCACCACATGCTTCCAGCAACCAGATAATTTT
CCTGATAATTAGATCAATAGCACACAGTGATAGAAGCCGTCTCGCCTCCCTGCCTGATGGGGTCAAATCAACCACACAGGATGATGC
TATATTTTTGACTATGCTCAAATATTTTCATGATTCTCATTAAGGCCAGTAGAGAGCCTTAAAATTATACTGGAGTTGTACCTAAAT
CCAATCCCTTTAAAAAGTCATTTAAAGAGATTATTCCAGTAGAGCCCACATTCCCAAATATTGTTGCTCACCAAAACGCCACCATCC
GTTTTCCCCTTTTGGGCTGTCTGGATAATGGCTCTCACTTGCATCCTTATGAACTCAGCTTAGGAAGAGGATAAACTCAAAGTGCAG
GGATGGGGTGATTAGCTGCTGCTCAGATTCCCCCACTGAATTGTCAGAGAGAAGGAAGCTCATTTCAGCCAGGCACAGTGATCCCAC
AACGCAAAAGGCTAAAGCAGGAGGATTGCCATAAATATGAAGCCAGCCTGGGCCATCTAGCAACTCCTGTCTTTAAAAAAAAAATTA
ATAAGCTCCTCTAAGAACAGAAAAGAATAAGGCTTCCAGGACAACAACAGGCCCAACAAACTAGGTAGGAGCTATTTCTTTAAGGAC
GGTTTTCAAGCGAGACTCTGGAAACAAATGGGAGCCTGAAGGAAGGTCAGATGTATGCCCTCCTGGGTGGCTGATCTTCTCTAGAGG
AAGAACCAGGACTCTGGGCGCTAACTGGGCATTGATCCCCAGAGAAACGCTTCAAGGTGACATGAATCTACCAGCAAACCCCACAGT
CTTAGTTGGGGATTTGAGTACTAGAGAAACTCTTCTGTGATTAACGTTTCAAAGCCATAAAAAATGTGTTAGAAATAGAGACAGGCG
GGAAGACAGAACAAAAAGAGACTGGGAAGGAGCAGGCGGGCCCTCGGGGAAGCCAGCACAGAGTTTGCGGTTGCCAAGGCCTCTCAG
CGCCTCAGCAGAATTATTTATGGCGTTTGATTGTTGGGTCTTTGATTGGGGGTTTCTTTAAAGAGCTGCCACAGATATAAAAAAGGA
ACCTGTACAAGCAAATCTTGACATATCATTGGCATACAGACCACAAGGAGCATCATGGCCCCGAGCCACCAGCCAAACAAAAGAAAA
CAGAGCTCAGGCATGACAGTATGTGACACTGTCACCCTGCAGCAACCATACCAAATGCTAGGAGGACAGCCTCAGCCTCAGCCTCAG
CCTCAGCCTCAGCCTCAACCTCCTCTCTAAGCCAGCCTCAGTCAGCCTCAGCATGGAGGGGAAGGCTAGGGTATGGGCAGTCGAAAA
TGCAAGTGTCATCCGGAGTCTACTCAGCTCACTGCTTTGAAACCCCACCGGCAAGGGGAGAGGGGTTTGTCTCCCAGATCTTTTCTG
AGGCTGTTTGCCAAGAACAGAAACAGTAACATTCCCTGTTTGCTCCCGTGGCGTTTTAGAGCCAAGAGTATACTTTGTATTTTCTTT
CTATTCCTTTGTCCTACCCCTTTTCCACCCAGTAAATCAGATACAAGAGGAAAATAAAATGAGGAAAGCCCGCTCTCTCAGCGGCTA
GGGAAATTAGCACACACACACACATACCCCGCACTATAATTGTAGAAGGAGATGAGCTTGTGGTTGTTAATTTCAATCTTTCAAAAA
TAATTCACTAAAATCCTCTTCAAGGCCACTTACTTTTGGAGGAATAGGGCTGAAAATCAAATCCATGGATATTTATTGCTTGCCTGT
GATGTGAGGTGCCTACTTAATTCAATAACATCTCACTTTTCTCCGAACACTCAAATCTGCTGAAATGTCCCGGTAATATCTTATATC
TTTTCCCAGGGAAATAAACACAAAAGGAATGATTCTCACAGAGGGAGATTGACAGCTGGCATGACAGAAATGTTACAAAGGACCTTC
TGGGCATCCTTGTCTGAACTGAAGCTGGCTCCGACCTCTGTGACCACTAGGGAAGCCTGTTCTTTGCCCTTTCCATATAGCCCATGC
ATCCAGTTAAGGTGACTCACAGGTCAAAGGCTGTTCCCAAGACTCCAGTGAATTCTCAGAGCAGGGTCCAGCGTGGAAGGAGTTGGT
AGTAGACGATTAGTGGCTTCCCCTGATGAAAGCAGATCCTTGCAAGAACAGGCAATATTAATCCCTTGAATATCTAAGCAGAAAGAA
AACATGAATTGTAGTCTGTGAATCACACAAATGGGAGTCATTCTGACATTAATTCATCCGGCCTGTCTAGCTTCCACCCAGTCCTTC
CAGGTAGCACCGGCTGTTGGCTTGTGTTTACTTGTGGAGAACGGAGGCAGCCTGGTAGCAATGGAAATGGACTCCTGACTTTCCCAA
ACTCCTGGCATGTCTCCTTTCACCTGAATAACTTGTCAACCTTGGTGGCAACGTCACTTCTGTACTTGGTGGGATCACAGCTTCCCA
CACTGACTTCGCAGTCACTGCCACTGTTAATCTATAATTATAACTTATTGCCAGCCCTGGACTACTCAATAATGTCTAAGGAAGTGA
CACATAGCAACGCTGTGCATGATCGAGAGATGACCCGTGTATCCCACCGACTCCCCACTCAGACCTGTGGTAGGCTCTCCAATCAAT
TTGATAAGTGTGTGCTGTTGGCTGGGGTGGCCTCTCTCTCCTTGCAGAAGGGTTCCTCGACCCCATGATCTCATCGAACCACATTTC
CCATGAAGTCAGTGCATAAAGCCAAACCCAGCAGCCCTGAACTACTTTAGGCTCCTGACTCTCCAGGGCTGCTTACATACGTCACTG
TCCCTTCACAGTCCAGGCCCAAAGACAGTGCCAGTTCAAGCTAGTCTGTACCTAGCAGTGCTCAGGGCCCCGCCCCTCACTCAGTTG
TTTACATGTGATGATCTCTCCAAGGCTCCTCCTGCCCCATCCTTAAAGCACCCTCCACCTGGGTTTTCTTGCCATAGACCTAATTAT
CCTGCATCTGCTCGTGGTCTTCCCAGTCATCCCTTTTCATCCCCCTAGACCTGATTCCACCCTGAACATACACATTCAAAAAACCTC
AGGCAGCCCGAGAACCTACTGAACAGTCCTAAATAAAACCTGAACTCTTAGAAAATGTCAAAGGCTTGAGGGAAAATGTTTCCACCA
GGCTTATTCAAAGACTTAATCTTAGCAGGGGTGTTTTATAGGGGACATAATGGCAGTGGCTGTTATGCAGACATGGAAGGCTGGCTA
AGGACCTCTAGGTGGATGGCCCATGGCTGGCATGGCTCATGAGTAGGTCCCCAGAAGAAGCGTTTCATTAGGTAATCAAGCATGCCA
GGACTAGATATGCTAATTCTCCCAGAAAAGGATTGCAAATGACATTCCCCACAAAGGCTGTGACCCAGAGAGACTGGGCACAGTGTT
TCTTTCCCCCACAATAATAGAAAAGCATAGAAACAAACAGGAAAGATCCTGGGGATGAGCTGGTCCAAACCAGATATGAATACTCAA
GTCCAGAGAGGGGGAGGGGGTGGATGCCCAACACGTGCACCTAGTTGGTGCCTGTGGAGGATCTGGTTTGCACACAGGAAGCATTAG
CACCCCAGTCACTACCCAGCATGGTTAGTTCCTTGAGCTTTGCATGATGGTTAAGGTCCCAGGCCTGCAAGACGAAAGAGCCAGCTT
GACTCCAATAGCCCAGTGTTACATCAATAGCACGGTGAGTAACCTGCCATTCATCACCGAGTTGCTATGGATAGAGCTCAAACTCCA
ACCCTGAACCACAGACTACATCTCCTTTCTGCTCCTCTCCCCATTCCAAAGTCCTGACAAGAATGTTGATGAAGCAAGCCCCTTGCA
GGTGAGGCAGTGAGCGTGGGTTATGATGGAGGAAGGAAGAGGGGAGGCAAAGCAGGTCGATGAGAGAGGGTCTTAGGAAATTGTCCC
TGGAGGAAAATGAGGAGAGAATGGCTGGCAGGAGGGGAGGGCGCACTGCTGTTTACAACAGACACCTCCAACCAAGCTCTGGAGACA
GGCTGCCTCCATTCACGGTCTCCCTCCATGATGTTCCTCATCCCTGCTCATTGGACTCCACCTATTGACTGTTGAGATGGTGACTCA
TGGCTGCCAGGCTGCTCTGGATTCCCTCTTTGCCCTCTCCAACCAAGAAGGACACTGCTCCCTCGGCGGAGAGGTGGCCAGAGCAGC
CTGTCGCATGCGATGCTGTGGTCAAGAATGCAGGGAACAGACACGTGTCCTTCTGCATACTTTCTTCTCTCGGGGAGGCTAGGGCCA
TGAGAAGAAGCTGACATGAATTGGAAAAGTGCTATCAATAACTTTTCGTAGGTCTACGAGATGGCTCGGTGGGAAAAGGCTCTTCCC
GCCAAGCCTGTTGACCTAAGTGTAATGAGCCCTGGGACCCACATGGAAGAAGGAAAGAATTGACTCTTGAACGGTATCCTCTAACTT
CTACCCATACACTGGGATGCATATACCATCCCATAATAAACAGATAAATGTAATGAGAAATATATGTTTACAAAAACAACACCAAAG
TAACTGTGCCTTGAAACACACCATGGGTGGTAAATAGAATGCTGAACAGTCTACCTGTGCTCTCATCTAATCAACACAGAAGCCCGT
CTCCAAGATGAATGACACTTGCAAGACCCTAACCCTCTGCCTGCTCTAATTTTCTTTGAAGCTCTTAGAATCTGATACTGTGGTTTT
TAACTTCGTACTTTCTCACCCAAAGATTACAAATTCTGTGACAGCAGCAATGGCATCGGTTCACCAAGAGTGGTAGCATGCACTGTA
ATCCCCAAAAGCTGCCACAGAAGGATTTTGTGTTTGAAGTCAATCTGGGCTAATAGTGAGTTCAAGGTTGGTCTAGACAACAGAGTG
AGACCAGGTTTTTTTGTTTTTAAAGATAACATTATTGATTTGTTGAACTCTGTCCTCTCCCTTTCTAGAACAGTGTCTGGCTCATCA
TAGGTCCTCGTTAGTATTTATTAACTATCCTTCAAATGGAGAGAGGACGAGAGTCTCAGGTAGCTCAGCAATAGATCCATGGAGGAT
CCACCCACTTACCACCTTTACCTGGAGGTTAATCCATGAAACGATGCAGACAGTCGTGCTCTGCAAAGCTTGGCACAGCAGTTAACT
CACAGTTATTTGGGAGATTTTCCTCAGACTTGGTTCTTCCCTAGTCCCACTAATGTGCAGAGTCTGTCCTTCCTAGCCAGGAAAGGT
AGTGTGCTTGTTTAATCCCAGTGCTCAGAAGGCAGAGGCAGGTGGATCTCTGTGAGTTTGAGGTCAACCTGGTCTACATATCAAGAT
CCAGAACTTCCATGATTGTGTGGTAAGACTCTGTCTCAAAACAAAACAGTCTCATTGTATATAGTATAAGGTATATGGTATATGGCA
TATGGTATAAGGTATAAAGTATTTAATATATGGTATATAGTATGTAGTATATGGCATGTGATATATCGCATGTGGTATATAGAATAT
ACAACTTAAAGTGAAAAGAGCAATGTGGGGATTAGAGAGTTTATAGACTCAGTGAACACCATCAGCCTCAGTTCTTATGCATCTGGA
AGCCAGAGAACTAACCATTCACACATAAAAGATCCCACCCAGCTATCTTCAGTGGCACCTCAACCCAACCAGTCAGAGCCAACCTTG
AATGGTCCTTGCCAGTTCGCTGCTTCCCTCTCAAGCCCTGACCCTCCCAACCCTGCATCCTGGGAACATCTTTTAGGTGTGGTATCT
TTGTTCATCAGGAGAGCTCAGAAGGTAGCTAAGCCTTCATCAGGGAAGGGTGTCTCACACTGTGGAGGCTGAGCAGGCCCCAAGAGG
GGCTGAAATGGTGCTGTCTTTTCAACCATGCTCAGAGCCTGGCACCAGGTGAGATGGAGGGAAACTCAAGAGAGCCAAGTGCAGTTA
AAAGTCTATTGCTTTTTCTGTTGCAGATATCGATCAACTTAGCTTTGTGCCAGGCCGTTTGCTAGCAATTAATTTCAGTCAGTCCAA
GGGCTTGAGTTTACCATTATTTTCCCGAATGCTCAGGGGCCTCCCTCTCAAACCCATAAAGATGATGTGATTCCGCTCTGCTCCCTG
GCCAAACCATGACTTCAGGATCTTTCATTTAGGCTGGCTGAAGGCAAGACTTGAAGTCTGTATTCTGTCATGTTGCTGAATGAGGGC
CAGATGCCTTAAAAAAGAAAGACAAAAACCAAACCAATGTCCTTCTCAATTCCTTGATCCTGGGAAAGAGGAAATGATTCTGTTTGT
TCAGATGTGATCCAGACCTGTCTTCCTTCAGACCATGTGGGAATTGTTTGCACCTCTTGGACCTGACTCTTGCTTAGACTTAGCCAT
TAGCTCTCTGTATGAGTCTGGCCTTTTATCCTCTTTCTGAAACTGTTTTCTTCTATTTTAAGTGGGGATAATAATAACCAATATCTG
TCTGATACTTTAAAATTTATATAGTGCTCAAATCCACACTGACTGTTTGCTATTGGCAAGCTGATGGGCTGAGGCTATGAAAAACCA
AAATGATGTTAGCCCGACATGATAGTGGGTTTGTGAGGCTGTCAGCCAGACCTGGATGGGGGAGCCTTGCTAAAACCAGCTATTCCC
TCCACACATCAGCTGTGCCTCACAAATGTGTGTGTCTATGTATGTGCCCAAGCATGTACATGTGTGTATGTATGTGTACCCAAGCTT
GTGCCCATGTATGCATTTGTGTGCATGTGTGCATGCCTGAGCATCTATGTATGTATGTATGTATGTATGTATGTATGTATGTATGTA
TTCCTATGTGTACCTGAGTGTGTGTGCCTGAGTGTATGTGCCTTAGTGTGTGCATGTGTGTGTGTCTGAGCATGTATTTGTCTTTAC
ATGTATGTGTGTACATGAGCTTGTATGCCTGTGTGCATGTGTTTGTGTACCTGAGCATGTGTGTATAGTGTGTGTGCATATATGCAC
GTGAATATGCCTGTGCCTGCATATGTGTGTGCATGTTTGTGTGTGCATTAGCCTAAAATGTCTTCCATTCACATTAAAGATAATTAA
GCATTTGCTTCTAAACTGCTTAATGATGCATACAGCCTATAGGCTTCCTCATTTCAAATGTTTGTTAGCAAGACACAGAGAGCATTG
TCAAAGGTTTGTCAGTTTGGCTATTCTGTGGCACTATCCACACTCCTAAGAAGGGACTGTATAGCTAGGGCTCTACCCGTCCTTGAA
ACCACTTGGGCAGACTTTGCCCTCTCCTGCTTGTCTTTAAGGCTTCCCTTGAAGGCCGAGCAGCCACAGAAACTGAAGAGGTCCTAC
ATCAAATTGCCCACCCCCTTAAGGGACAACAGCAGCATTCTGCTGAGAGAAAGCCCTCCCTTCCCCACCACCTACTTAGCTAACCAG
ATCCAAGGCCCAGAGCCCATCATCTGCTTGCCCTCCCTTGGTAGCCAGACACTGGCTACCTCCAGCCTCGTCTTACTGCCACCTGCT
CCCTGTCAAACAAGAAAGTGGCAGAGTGGCCAATGTAACCACTTAAAACCATTTCACAGTAGATTTTTTCCTGAACAACAACAAAAA
AGTAATATTTATGTCACTTAGACATTTCTGAAAAGACTTAAGATGTTCTTTTAAAAACATCACATCGACGCCAATGAAGAATACAGC
CCAGCTTTAGTAATAGATTTTGAAATACATACAAGTTATCAGTCTGCAAAGCAGTGTTTCTTAGGCCTTCCCACGTGTCTCAGCATG
TTAGCAACGCCCCCATCTGTTGGTGTCAGTCTCAGAAGCTGATTTGTAGTCTCTGATTTTATTAAAATCCTATCTGGCAAGTCACTT
GATCTCTTCTAGTCTGGTCTCCTCACTTCCATGCCCTGGGCCTGGGGTCCTGTGCCAGTGTGTATAGCTGACAGAAATGGAGACCAT
TAATCAGAAATACATTTAGCTTGTGCTTAATAAGGAGTATTTTCTCTTGTTCTCGCTAGTAATTGCTGAAGATGTCTGTGTTTGGAT
TAAAACTTTGTCCCCTGCCATCTGGATGCCATTGATACGTCTCAGCTCAGTCATGAGCTGGATGATATCAGTGGTTTCCATGTCAAC
AGATGACAAGAGTTGTGTATGGAGCCGTGTGACTACCGTGCCTGGGGAAGCCAGCCCCGGGAAGGAGGCAGCTGTGCTGTCACACCT
AAGCATCTTCCCTGATCTCAGACACCACTGAGATGATTTCAGTTTTGTGCCAATTACATTTACATGTGAAATCGCAACGCATAATTT
TTGGCCAGTTAGGAAAGGAACAGTGGTCTTGTTGGTACCTGTCATTGCAGTTTGTAAGAAAGACACTTTCTCAAAGACACCTTTTAA
AATCTCCACTTGGTAGGAAACCTATTACTTTCAAGTAAAGTGTGGTACCCTGATTATGAGGGCATTGACAGCATGTATTTATTGATC
CAAACTGTACCCCAGACTTCAAGAGCTTGCTAAGCAGCCTGTCTAAATATAGCATCATCAGCGGCAGCCGAGAGGCAGCAATGAAAC
CATTAAGAAGGAGGAGAAGTGCCTCTCGGCTCGAAGCTCCAGTCTAGGCCTCAGATACAAGATTAATAAAAAATGTGTTTTAATCAT
CTTTTCTAAGAGTTTCCTGATCTTGATTAAGAAAGTACATCTCTGAAGAACCATTTTCTCAGGAAGTAGATGGTAGCCACACATGCA
AGGAAAAAACATATTGTGTGAACCCAAAAATTAACCCAACAGCTGCTTGTTACCTTTCCCTCAGATCAAGAATGCCGCCGCCAATGT
CCTCCGGGAAACGTGGCTAATCTATAAACATACAAAGCTGCTAAAGAAGATTGACCATGCCAAAGTCAGGAAACACCAGAGGAAGTT
CCTCCAAGCTATCCACCAGTGAGTATCGGAGAACCAGAGCAGTGTACTGTTTGGTCAAANNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNTCTCTTCTTGAAAATCAGCACCGTGGGCAGAACTAAGCGTGGG
TAGTCTGACCAGGAGGGCTCCCTGCCCCCCACTACCTTCTGATAGAAATTATACAGCAAATGCATAGCCCAGGGCTGGGCTGGCAGA
ACATTCCAGCAGATGTCAATTGTAACGGAACCTTATTCCCTGGGAAAGCAAAGGAAACTCCCTCGGGCCAATTAGGCCCCCTTTGAA
AAGAGCCTGCAAGCAGAAATTATTGGTTGCTGGGCCACGTGCAGGCTCCTCATTCAATAGAGGGATCAAGAAGGCAAAGAAAGAACT
TCCTATGGCAGTGAGGACCTCTGTCACAGCTAGGGAGACGCCCACAGACACATGTGCCATTGGAAGGGGCATTCAGGTGGAGTGTGG
GGTTTCCCAGAACCCAGTGTGGGAGTGGATGAATGGACCTCTGGAGTCTTAGGCATGTTGGTTAATACACAGAGTTCCAGCAAACCT
ACTGGAAGTACTCCCCATATGTTAGAACCACTTATTCCCAGGAGATAAGGGGGTGTCTGGAGACACGTATGCGCCACCTCCCCTCTC
CTGCTCAGGTGCTGTTCCCCCACGCCAACCTTCACCTCAACCCCCACCGCACCCCAGGTCACCTTGGGGCATATTTGACTTTCATTT
GTTCTTAATTCTAAATTCTTGAGAGAGGTGATTTGATTGGCTGGTTTTACTGACGTGTCTGTCTTTAGACAACTCAGCTATGACTAG
GAAGAAGGAAACAGCTAGGACAGGACTCTGTGAGCCTCAGCAGTGGCTCAGTGAGATGCCCCAGGCAATGTTTCCCATCTCACCCAC
AGGGCACACTGTGGCTGATGCTCCTCCAGCAATATTAGGTTAATCTAGGTTTTGAGTTCATCTTCCTGCAGGGCTGCCCACAGTTTC
TCATTGACGGAGTGAAAGCAATGGCTTGGTAGACTTGGAACACTTGCTGGCAGAGAAAGAGGATCTAAATTTGAATGCCCAACACTC
AGAGCTGCATGTGCCTTCAGCACTGGTGGGCAGAAAAAAAAAATGAAGTTTGATGGCCAGTGAGCTTGGCCAAGACAGCAGTTCCTA
GTTCAGTGAGAGGCCCTGTCTCAAAGGAATAAGACAGAGAATAATAGAGAAAGAGACTCCATATCCTCTGGGCTTTCACATGCTTGT
ACCTATGAACTGGCACCCACAGATTCACATGAAATACACACACAGACACACACACACACACACACACACACACAGAGAGAGACAGAG
AGAGAGAGAGAGAGAGAGAGAGACTCCTGCACTGGACTGGGAAATAAAGCCATATATCACAGAGGAATGAAGTCTGTGGGAAGAGAG
TGTGCACAGAGCCATTGTAAAGTCCATGCGTGAAACCGGAGGAGGAGGCAGACCCTGCTAACCATCCATTTAGTCCTCCCATGAGAC
CCAGATTCCAGTGGCAGTCACGTGTGCTTCGGTGTAGGTCCCCGCTCCCTTAGCATCCTGTTAGCATTGCGAAGGGAAAGCATCCAT
CGTAATGCTATGCTGGCTCCTTTTCCCGCCGTGCTTTGCCTCCCATCTCCCCGCCCCTCGCCTCTGTTCTTTGCTCCTCCCTAGATT
AAGAATGTACCCGTCCACTGCATGTTCTGTATTAGACAGGCCTCATGTCATGTATGCTGTCAGAGCTGGGAGGGCTGGGGCCCCTCA
CCCAATTCCTTCAGTTTCTTCAACTGGGAAATAAAAACAGTTCCCACCATCTGAAACTTGGGTGGTCAGATAAGAACTGTAAACCTG
CTTTGTGAGCTGTAAACGTTTCAAAAATGGCAGTTCTGATTAGGAACACAGATAAGGAAGGAACCAGAACCTAAAGGAACAAGATGG
CCACCACCTGGTTCCACACTGGAGTTCACCAGGTGCTCTTGTGGCCCAGCATGGGATCCGTGGCTTGTCCATTCCGGAGGGACCATG
CCCTGTGAGGAGGCACTTGACCACCCCCACCCCCATCCAGGTTACCCTTTCCCTGGGAGGGCAGAGCTGACAAGAGAAGAGCTGTCT
TCAGACCTGCTCCTCCAAAATTCCAACTGCAGGGGGCTCTCCTGCAGTTTCACCATGGCTCCTCTGCTGCTGCCTCACGTTCTGTTC
TCCTCCCTTGTCCTCTGTTCCTCTCTCATCCTGTTGCCTGCTCCTTCCACACATCCTAGAGCAGTGGTTCTCAGCCTTCCTAATGCG
GTGAGCCCTGAATACAGTTGCTCATTTGAGGTAACCCTAACCACAGAATGATTTCATCTCAGCCTCATAACTGTAATTTTGTTCTTG
TTATGTATGAATCGTCATGCAGGATATCTGATATGCCAGCCCCAAAGGGGTCTTGACCCATAAGTTGAGAACTGCTGTCCTAGAGAA
ATGCCAGGTCAGATGAGATTAGGGTAATTTCATACACACACCACACCACACCACACACACACCACACCACACCACACACACACCACA
CCACACCACATGGGCACGGGCATCAGAACCCCCTTTCCTCTCTCTGGGTGTGAGACTTGAGAGCCACCATTCCCGGTGCTTGTTGAT
TGTCTGCCGTGTATAGTCTAGGCTTCTGCAATCAGCATAGGAATCACGGGGTGTCTGTGCAGGGAGCTCTGATAACAAGTGGGACCC
AAGAGCAGTGTGTGACAGTGTTGGAGACAGGAGGCCATCAGGAGCACCCTCAATGCAGCCTTTGCCAGGCAGCATGGGAGGCGGGCC
TGCTCTGAGGAGTCAAGTGTCGTCTGCCCAACCAAAACCTCAGCCAGGTCAGACAGCCCTTAAAAGAAGCAACCTGCTCCAGGGCTT
CTCTACCTCCTCTGGAGCCAAGGAGCAGACGGGTGGCTCGTCAGCAGGGCAACCCCTTGTGGTCCAGGATCATGCCTGAGGCATTAA
TGACATTTTATATTCTGACTGCTGCATAGAAAGGGGGTGGGGATGCAATCTCATAACTATGTGTAGTTATTAACTCTGATTTTATAA
GGAGGTCAGGGTAGGAGAGGTTAAGTGGCCTACTAGTGAGCTGAACTTGCACTTGAACCTTGACTTGTTAATTAGGAGTCTACAGCC
TTGGCCATTCTCACCTCAGTGTCTCCTCTTGAATCATTAGACGGCAGCACTCTGTGTGAATTGACAACCATGTAGAGCCATATTCAA
TTTAAGAAAGGGACGTGTTTCTTTTCTTCCATGCATATTTTTTATTTAAAATAGATAACTAAATTGGGCGTGGTGATGTATATCCTT
AGTCCCCAGCACTCAGAAAGCAGAAACAGATCTCGGTGAGTTCAAGGCCAGCTGGGTCTACAATACTGAGTTCCAGGACACCCACAA
GTATGTAGTGAAACCCTGTATCAAAAGAACAAAACAAAACAAATTATAAAATTGATCCTCGATATCCATTCCTTTTAGAAAAAAAAA
GGGGTGCCCACCCCAGCCTGTCCATCACCTGGGAAGTCCTGGAATTCTCCTTTGGTGACACGGGAGGAAGCTGGGCGGAGGTGGCTC
CAGCTGCAGGGTGGAGGGCATTTCATCTTCCCACAACAGAGTCCTCTGTGGTGATGGTCTTCTTCCTGCCTCCCCCTCTGTCTCTCC
CAGATGCAGAACGTCATGTATGACTTAATCACGGAGCTCAATGACCGGAGTGAAGATCTAGAAAAGCAGATTGGCAGCCTGGAGTCC
AAGCTGGAGCACCTCACAGCCAGTTTCAATTCCCTGCCCCTGCTCATCGCAGATACCCTGCGCCAGCAGCAGCAGCAGCTGCTCACT
GCCTTCGTGGAGGCCCGGGGCATCAGTGTGGCCGTGGGAACTAGCCACGCCCCTCCCTCTGACAGCCCTATCGGGATCAGCTCCACC
TCTTTCCCAACCCCATACACAAGTTCAAGCAGTTGCTAAATAAAACTCCCCACTCCAGAAGCATTACCCATAGGTCTTAAGATGCAA
ATCAACTCTCTCCTGGTCGCTTTGCTGTCAAGGAAGTTGCAGACCAGGGAACAGAACGAAGAGAGA
MOUSE mRNA SEQUENCE:mR27-009.1 (Seq ID No: 24)
GCTTTGGGGTGGAGGGTCGTGCTGCAGGGAACTCAGCCAGGCCCCAAGATGGACACTTCTGGGCACTTCCATGACTCGGGGGTGGGG
GACCTGGATGAAGACCCCAAGTGTCCCTGTCCATCTTCTGGGGACGAGCAACAGCAGCAGCAGCAACCGCCACCACCGCCAGCGCCA
CCAGCAGTCCCCCAGCAGCCTCCGGGACCCTTGCTGCAGCCTCAGCCTCCGCAGCCTCAGCAGCAACAGTCGCAGCAGCAGCAGCAG
CAGCAGTCGCAGCAGCAGCAGCAGCAGGCTCCACTGCACCCCCTGCCTCAGCTTGCCCAACTCCAGAGCCAGCTTGTCCATCCTGGT
CTGTTGCACTCTTCTCCCACGGCGTTCAGGGCCCCCACTTCAGCCAACTCTACCGCCATCCTCCACCCTTCCTCCAGGCAAGGCAGC
CAGCTAAATCTCAATGACCACTTGCTTGGCCACTCTCCAAGTTCCACAGCCACAAGTGGGCCCGGTGGAGGCAGCCGGCACCGGCAG
GCCAGCCCCCTGGTGCACCGGCGGGACAGCAATCCCTTCACGGAGATAGCTATGAGCTCCTGCAAATACAGCGGTGGGGTCATGAAG
CCCCTCAGCCGCCTCAGCGCCTCTCGGAGAAACCTTATCGAGGCTGAGCCTGAGGGCCAACCCCTCCAGCTCTTCAGTCCCAGCAAC
CCCCCAGAGATTATCATCTCCTCCAGGGAGGATAACCATGCCCACCAGACTCTGCTCCATCACCCCAACGCTACCCACAACCACCAG
CATGCCGGCACCACGGCCGGCAGCACCACCTTCCCCAAAGCCAACAAGCGGAAAAACCAAAACATTGGCTATAAGCTGGGACACAGG
AGGGCCCTGTTTGAAAAGAGAAAGCGACTGAGTGACTATGCTCTGATTTTTGGGATGTTTGGAATTGTTGTTATGGTGATAGAGACC
GAACTGTCTTGGGGTTTGTACTCAAAGGATTCCATGTTTTCGTTGGCCCTGAAATGCCTTATCAGTTTATCCACCGTCATCCTGCTT
GGTTTGATCATCGCCTATCACACAAGGGAAGTACAGCTCTTTGTGATCGACAATGGTGCAGATGACTGGCGGATAGCCATGACCTAC
GAGCGTATCCTCTACATCAGCCTGGAGATGCTGGTGTGCGCCATCCACCCCATCCCTGGAGAGTACAAGTTCTTCTGGACTGCACGC
CTGGCCTTCTCCTATACCCCATCTCGGGCAGAGGCTGACGTGGACATTATTCTGTCCATCCCCATGTTCCTGCGCCTGTACCTGATC
GCCCGGGTCATGCTGCTCCATAGCAAACTCTTCACTGATGCCTCCTCCCGAACCATCGGGGCCCTCAACAAGATCAACTTCAACACC
CGATTCGTCATGAAGACGCTCATGACCATCTGCCCTGCCACGGTGCTGCTGGTGTTCAGCATCTCTCTGTGGATCATCGCTGCCTGG
ACTGTGCGAGTCTGTGAAAGGTACCATGATCAGCAGGATGTAACTAGTAACTTTCTGGGTGCCATGTGGCTCATCTCCATCACGTTC
CTTTCCATTGGCTATGGGGACATGGTGCCCCACACATACTGTGGGAAAGGTGTCTGTCTTCTCACCGGCATCATGGGTGCAGGCTGC
ACTGCCCTTGTGGTAGCTGTGGTTGCCCGAAAGCTCGAACTCACCAAAGCAGAGAAGCATGTGCACAACTTCATGATGGACACTCAG
CTCACCAAACGGATCAAGAATGCCGCCGCCAATGTCCTCCGGGAAACGTGGCTAATCTATAAACATACAAAGCTGCTAAAGAAGATT
GACCATGCCAAAATGCAGAACGTCATGTATGACTTAATCACGGAGCTCAATGACCGGAGTGAAGATCTAGAAAAGCAGATTGGCAGC
CTGGAGTCCAAGCTGGAGCACCTCACAGCCAGTTTCAATTCCCTGCCCCTGCTCATCGCAGATACCCTGCGCCAGCAGCAGCAGCAG
CTGCTCACTGCCTTCGTGGAGGCCCGGGGCATCAGTGTGGCCGTGGGAACTAGCCACGCCCCTCCCTCTGACAGCCCTATCGGGATC
AGCTCCACCTCTTTCCCAACCCCATACACAAGTTCAAGCAGTTGCTAAATAAAACTCCCCACTCCAGAAGCATTACCCATAGGTCTT
AAGATGCAAATCAACTCTCTCCTGGTCGCTTTGCTGTCAAGGAAGTTGCAGACCAGGGAACAGAACGAAGAGAGA
MOUSE PROTEIN SEQUENCE:mP27-009.1 (Seq ID No: 25)
MDTSGHFHDSGVGDLDEDPKCPCPSSGDEQQQQQQPPPPPAPPAVPQQPPGPLLQPQPPQPQQQQSQQQQQQQSQQQQQQAPLHPLP
QLAQLQSQLVNPGLLHSSPTAFRAPTSANSTAILHPSSRQGSQLNLNDHLLGHSPSSTATSGPGGGSRHRQASPLVHRRDSNPFTEI
ANSSCKYSGGVMKPLSRLSASRRNLIEAEPEGQPLQLFSPSNPPEIIISSREDNHAHQTLLHHPNATHNHQHAGTTAGSTTFPKANK
RKNQNIGYKLGHRRALFEKRKRLSDYALIFGMFGIVVMVIETELSWGLYSKDSMFSLALKCLISLSTVILLGLIIAYMTREVQLFVI
DNGADDWRIANTYERILYISLEMLVCAIHPIPGEYKFFWTARLAFSYTPSRAEADVDIILSIPMFLRLYLIARVMLLNSKLFTDASS
RSIGALNKINFNTRFVMKTLMTICPGTVLLVFSISLWIIAAWTVRVCERYMDQQDVTSNFLGANWLISITFLSIGYGDMVPHTYCGK
GVCLLTGIMGAGCTALVVAVVARKLELTKAEKHVHNFMMDTQLTKRIKNAAANVLRETWLIYKHTKLLKKIDHAKMQNVMYDLITEL
NDRSEDLEKQIGSLESKLEHLTASFNSLPLLIADTLRQQQQQLLTAFVEARGISVAVGTSHAPPSDSPIGISSTSFPTPYTSSSSC*
MOUSE PANTHER CLASSIFICATIONS
              FAMILY (SUBFAMILY)
                            CF10153 (POTASSIUM INTERMEDIATE/SMALL CONDUCTANCE CALCIUM-ACTIVATED
CHANNEL)
              BIOLOGICAL PROCESS
                            Transport (2.15.00.00.00) > Ion transport (2.15.01.00.00) > Cation
transport (2.15.01.01.00)
                            Neuronal activities (2.18.00.00.00) > Synaptic
transmission (2.18.01.00.00)
              MOLECULAR FUNCTIONS
                            Ion channel (1.03.00.00.00) > Voltage-gated ion channel (1.03.03.00.00) >
Voltage-gated potassium channel (1.03.03.03.00)
MOUSE GENE ONTOLOGY
                            No Gene Ontology
MOUSE PROTEIN DOMAINS (INTERPRO SIGNATURES)
                            IPR003931 (SKCHANNEL)
                            IPR003931 (SKCHANNEL)
                            IPR004178 (CaMBD)
                            IPR003931 (SK channel)
                            IPR000694 (PRO RICH)
                            NULL (GLN RICH)
                            IPR001622 (CHANNEL PORE K)
                            IPR000694 (PRO RICH)
                            NULL (GLN RICH)
                            IPR001622 (CHANNEL PORE K)
HUMAN GENOMIC SEQUENCE:hD27-009 (Seq ID No: 26)
CTTACAATGTAACAGTGGCAGGAGGAGGCGAGCGAAGCTATTGAGCCAGCGAGGAGTGAAGCTGAGCCTGGCCTCACACGCTCCTAG
AGGACCACCTCCTGAGAGAGTTCTTTCACCCCCTCTTCTTTCTCCAAGCTCCCCTCCTGCTCTCCCTCCCTGCCCAATACAATGCAT
TCTTGAGTGGCAGCGTCTGGACTCCAGGCAGCCCCAGAGAACCGAAGCAAGCCAAAGAGAGGACTGGAGCCAAGATACTGGTGGGGG
AGATTGGATGCCTGGCTTTCTTTGAGGACATCTTTGGAGCGAGGGTGGCTTTGGGGTGGGGGCTTGTGCTGCAGGGAATACAGCCAG
GCCCCAAGATGGACACTTCTGGGCACTTCCATGACTCGGGGGTGGGGGACTTGGATGAAGACCCCAAGTGCCCCTGTCCATCCTCTG
GGGATGAGCAGCAGCAGCAGCAGCAGCAGCAACAGCAGCAGCAGCCACCACCGCCAGCGCCACCAGCAGCCCCCCAGCAGCCCCTGG
GACCCTCGCTGCAGCCTCAGCCTCCGCAGCTTCAGCAGCAGCAGCAGCAGCAGCAGCAGCAGCAGCAGCAGCAGCAGCAGCAGCAGC
CACCGCATCCCCTGTCTCAGCTCGCCCAACTCCAGAGCCAGCCCGTCCACCCTGGCCTGCTGCACTCCTCTCCCACCGCTTTCAGGG
CCCCCCCTTCGTCCAACTCCACCGCCATCCTCCACCCTTCCTCCAGGCAAGGCAGCCAGCTCAATCTCAATGACCACTTGCTTGGCC
ACTCTCCAAGTTCCACAGCTACAAGTGGGCCTGGCGGAGGCAGCCGGCACCGACAGGCCAGCCCCCTGGTGCACCGGCGGGACAGCA
ACCCCTTCACGGAGATCGCCATGAGCTCCTGCAAGTATAGCGGTGGGGTCATGAAGCCCCTCAGCCGCCTCAGCGCCTCCCGGAGGA
ACCTCATCGAGGCCGAGACTGAGGGCCAACCCCTCCAGCTTTTCAGCCCTAGCAACCCCCCGGAGATCGTCATCTCCTCCCGGGAGG
ACAACCATGCCCACCAGACCCTGCTCCATCACCCTAATGCCACCCACAACCACCAGCATGCCGGCACCACCGCCAGCAGCACCACCT
TCCCCAAAGCCAACAAGCGGAAAAACCAAAACATTGGCTATAAGCTGGGACACAGGAGGGCCCTGTTTGAAAAGAGAAAGCGACTGA
GTGACTATGCTCTGATTTTTGGGATGTTTGGAATTGTTGTTATGGTGATAGAGACCGAGCTCTCTTGGGGTTTGTACTCAAAGGTAG
GGGCTGTGGTTTCTCTTTATACCTTGAACAAAAGGAATATGTAGGTAGCAAGAGAGGGATTGAGAGAGGGGGATTGTGAGAGAGAGA
GATTGAGAGAGAGAGAGAGAGAGAGAGAGAGAGAGAGAGATTGAGAGATTGGGAGGGAGACTGGGAGAGAGAGGTGGTGGTGGTGGT
GAGAGGCGCTTGCTCAGTTATATTCAACACTCTAACTTCCCGTGGTTTTCCTTCTTGATCCCGGGAGAATTCATTCCTAGCTTCCTC
TGGGGGGTAGGGGACATCCCCACACACTGTGTCTAATTTGCAGACTCTCTGTTAGGGAGGATTCAATAATCCCTTCTGAGAAATCGC
TTTTCCTCCTGGCTCACTCGTACTAAAGCATAACCCAGCAGCTTAACGCACCAATTAATTACACTCCGCCTTCATGTGTTTGCCAGT
TCCTGCGACTTCCCCTTCTTTCCTCCCCTCCCTGCTCCACTCCATTTTCCTGAGGATAAAAAAGAAATAAATGTCTGGGTGGGGGGT
GGGGATTCCGTACCATTGCCCAAGTCCCTTCTCTCCCTGTCTTCTCCATCTCTTCCCAAGTTTCTGTTTCCCTCCTCTTCTCAGAGA
GTCTCGGGCAAAATAGAAAACCATCCATGACCTGGGTCTCCTGAGGGCATGGGGTAAAAATATAGGTGGAGGAAATCAGTGGTCCTT
TCCCTCAGGAGAGAGGACTCAGAATAAACCTGCTGCTGCTTCGTAAACATCTCCTGATAAAAATGGCTACAGGTGTTACCTGGGCAG
AGCAGCTGGGCGCCGCCTTGGAATCAGCTGGGGGAGCACCTCTCCTTGGGACGGGATGACAGGGCTTCCCAGGCGGTCCCCACAAGC
CCGCGCCCCAGCTCAGCCCCAGTTCTCCCCTCCCACCTCAACTCCTCCTTGGGATAAATAAAGATGAGTGTGTGTGTGAGTGCGCGC
CCGGATGGAGAACAGCAGGCACTGGCTTTAGCGGGGAGCTGGCCCCACTGCTCCAGCCTCTCAGTCCAGCCCCAAGACGGAGGAGGG
GGTTTCCCTCCCAGAGGGAGTGGAGATGGAGGAAGGTGGGTTTCTGCCGATGCTCTGCTTGCCGGGGAGCTCTCCTGCTGTGGGCTG
GAAGGCTTGGAGACCTGGGAATCGGTGGACAAGTGGCTCCACGATACCCCCACCCCCAATTTGTTGGTGTGTGTGTGTGTATGTGCG
TAATGTTTGTACGTATGCATGCGCCTGCTTGTTGCTTGGGTTTTTCTATTTATTTGTCATTTGGGGCAGGGGGTGGCTTGGTGGGGG
GGTGGCTGGGCGGGCTGGCCATCGGGTAGCTGGGAGGGGTTCAGCTGTTGGAGGTTTCAAATAGAGCTTGTTTCAGGCAGGGCACAG
CTGCTGCGAGACTGTCACATTCCTTATTGAAATCCAGCCGCAAAAACCTAGGAGGTGCTGATCGGATTTCCCCCATTCCCAGCATCT
CACTTTAATCGGTGATGGGCAGACTTTCCCGCCCTGGAAACGGGTGGGGCAGACCTGTCAGACGACCTGAATAGCCAGGACCAAGAC
TTCCAGGCAGAGGGTCCTCCAGCAACCCGGCTGCTCTCTCAGTGGGCTGCCCCAGCGGAGGGCAGCACCTGGAAGCCGCCTTAGTCA
AAATAGCTCAAGGATGAGCGTCTCAGCGAGCAGAAGCGAGTGGGGTTTCCCTTGGAAGCGCAGCTCACTTAGTCATTCCCTGTAATG
AGCCTCTGCGTCTGCGGGTCCTTCTGCTCGCTGCCTTCTCAGAACCGCCTCTCCTGCTGGGCGCTCCAGGATATGGGGCTGGCAGCC
TCGCTAGGACTGGCAGCTGCCCTGGTCACAGCCCTGGCTCTCTCCAGTCCTCCCTTGGAGACCAGGGGCCCTCACTGGGGAAAACTG
ATGAAGGCCCAAATGCAAGGGGGGTGTGGGGAAGGGGCAATCCCAGCACCTTCTCTTTCAGTGACACCCGTAGTCAGAGTTCTTAAG
GAAACGGCTATTCCCAGAGACAGTGGCTGTGCACAGATCCCAGTGTGGGATCTATGCACATTTGTCTTAACGCGACGGGAGAGTCAC
CACTCTGCAGGATCAGGCCCTCTTCCCTGCCTGGGGCAGGTGCCACCCCTGAGGGGGAGCTCAGGGAAAGGGGCTTTCCCAGAGATG
CGGAAGGCAGATTTTAGGTGAATGAAAGGGGAGGCCACCTGAGGTGCTTGGCTCCCCGCATTTCTGAATCACAGACCCTCATAGCAG
CAGTGGCGTGCACCGAGGGGGCAGTAGCTGAGAGGTCCTTCTTGCCTCAGCGGCCACTTCCTGTGTGCCTTGGGGCTGGTCTTGAGA
TAAGCAGAGCTCATTTAACTTGCTCACTCATTCACCCATCCCTGCACTGTCCATCCATGTTTTGAGTCATTTATGAAACAAGTATTT
TCTGGGTGTCTCCTATGTACCAGGCCTTGTACACACTGCAGGGCCAAGATAGATATCCCTCTCTCCTGGAGGCAAATGGGAACATTT
CAAGGAACTGGGCCAGAGTTGGAATCCCAGCTCTGCCACTACTATTTGTGTGATCTTGGATAAGTCATGGAGCTTTAGCGAGCCTGT
TTCCCCAGCTGTCGAGTAGGGAGAATTAAATTAGAGACGTGTAAGGCACGTATCTGCCTGCTCAACAAATGGGACCTATTGTGATTG
TTACTGATGATGACAATGATGAGAGGAAAGGAGATATCTTTTTCCTGTTATCTAACTCTGGCTGTAAAATTAGTTGGAGCTTTAACA
GATTGGGATCTAAGACTTGTCAACGGAGTGAGAAGTTGTATCTGGGAGGGAAGAAGTCCTGAGAAGGGACTGGGTCTGACTAGAGAC
CACCTTTAAGCATGGTTTATTCTTGGGGGGAGGTGAATATCTGTGTCCCAGTTTCATGGAGGAAGTGGTCTCTACTCCTCCATGGTC
TGGGTTAGTCACAGCCCAGGAGAGCTCAGGGACATTCAGTGGACCCTGGGGAGGGATGGGTGACACTCTTTTGCTGGAGAGCTTTGT
GTGCCTGGTTGGCCCTGCTGACCAGTCTGGAGGCAGGCGGATGGACAAGGCAACCTCAGGGGTACCAGGCAGATTAGGTAATTCTGC
CCATCCAGTGGCAGCTCCCACTCAAAGGCCCTTGGGAGGTGACAAACTTTTTTTTTCCCCAGGTGATTGAAGATTAACTAACCTTGT
GCCTGAGCTGCTGCTTTCTGAAGTTCTCTGCTTTTTTTTCCGTGGGGTTGTGGCCTTGCTGTTCACCTATGTTTGGCGGGTGGCTGC
TGCTGGGGAAGTAACTTGTCATGCAGGCCTGGGTCTTTTGAAAAGGAGGGTGGCATGTGCCCTGAGCATGTTCAGTTAATGCAAATG
GGAGCCAATGTGAGGGGGTTGAGCAACCGGGACAGAACATCCCCAGCTCCTGCCTCACAGGCCTGTAGGTGCTGATGATGGAAGTAA
GCTCTGCTAGGAGGCATCAGCCTGGAGCAGGGAGGTGGGGCTTGATTCCTGGGTGAGCTCAGATAAGAGGAGAGCTTCTCATTATTA
TGCCTGGGACATGAGATCAAACGGCAGAGATGCCTGAGATGGCCCCAGTGGGCTCCTAAAGGTACCAGGTGACCCTTGAACCAAATG
AGGAGTCCTTTTTTTTTTTTTAATAGGCAGGTTTACTCTCTGAAGTCTTAGACATTGTGACGAGGCCTGGTCTAGAGATTCTGTTGA
GAATCTTAATCACATCATCTGTACTGATCGGTGCCACCTGGGTAGACCAGCAAGGACCTGGGGAGTCCTTCTTAGTGTTCTTTATTT
TCTTTTAGAGCTGGTTTGGGGCACACCCACTCCATTGCATTAAATTACCTGTATGCTCTCTTCCCTGGGTTCAGAAAGAATTAGGGA
AGGGAAAGGCCTGGCTGCCCTCCCCTTTCCCCAGGCTGTGACTTCACCACCCTGACCCTGCCATCATGTTGGTGGCTGAGCTGGTAC
CTCTGTGTGGCTGGCAAGGCACCCAGGGGCCTTGTTTCTCGGGATCTCAGGCGTTGGGCTGTGGGATCTTCTCAGGGGAGGCTGGAG
AGTGCTGGTGTGGCAGCATGGCTCTTCTTTTCCACCAATCTGTACAACCTCAAGCCACTTCCCCATGCCCGGCCTTGAAACTGGTCT
GCATCTCTCCTCCCAGGCAGAACCAGTCCTCTGCCCACTCCCCCACCATGCTCCTGTCTCGCCTGCCCCACTGATTTCCTGCCCCTC
TGCTCCTCTCTGTGGCCCCTGCCTGGCCTCGTTGCTGGGGTAACCCAGGTGGCAGAGAGGAGTTGGTGTGAAGATGCTTATCCAGGC
TGCCCTGTCTGCTCCCACAGTGTCTTCTTTTCCCACTTCTAGTTACTCTTTCTTCAGACAACCTCATGGGAGGCCGCTTCTCCGTAT
GCCCAGGGGAGCCTGGTCGTCTGGCCATTCACTCTTTAAACAGAATGGCCAAGGCCAACTTGTCTGTCTCTTTGCAGACCTCACAGA
GGTCCCTGAGTCCATCCCCAGCCTCTAGACCAAATGATTGAGTAGTATCACTGGAACCAGTTCTGATCCTCTGCTCTCCCTGGTCAC
GGAAGCCTTGTCTGCAGAAGGGCAGAGCAGCAGTGCAGGCTTGGCTAGCCAAGTGGCTCTGCACCTTCTGTTTGGACCCAGGTGCTG
CTTTGGGATAGGTTGTTGGCTGTCACAGTGTCCTGGTATTTTTGAGAAGATGCCTCTGGGTACTGGTGCCTGTGTTTCAGGCCATGG
TGGAAAAGTAGGAGTAGGATAGACCCTTTCCAGCCCTTTCCATGGACCCTCCCTGTGGGGTGACCTGCAGGACCAGGATGCAGAAGG
CAGGAGACTGGCTGAGAGCCTGCGAGCCCACTGCCGAGGGCCCTAGGCACACATTTTGTCTTCACAGGTCACACTGAGTGTGTCGGC
CCACAGGAGGGCAAGCGTGAAAATGGCCTTTTGACAGCGTGAAAATGGCCTTCTCCGCCACCCGCCCCAGGGTGGTGGTGACTTTAT
TTATAATCTTTGTGGACAGTCTGTAAATGAGTCAAACATCTCTAACCCACAGGGGGACAGCTAACTTTTTATATATGCTTCTCCTTT
GCGTGTCATCACACTATTTATAACTGTCCTGGGGAAAGACTTCATCACACATCCGGACTTCCCTTCCCCACAGGCGGCATTACCCAG
GTGCTCCGCCAGGGGCATCCTGGCTGGGGCACTTATCTCCTGCACAGCCTAAGTAGAAAGGCGGCTGATGCCTCGTATTTCTCTTAT
TGTGCTTTTAGGAAGAGGGATTTGTAAGGCCATGGTCAAAAGATCTGCTTTAAAAAAATATAAGGTAAAGAAAGTCAAAATGAAAAG
AAATTTGAGTAACCTCTGTAGAGAATCTGGGGCCTGGATGGAAATGTAAGATCAGAATAGAGCGCTTCAGCTCTGTTGCAGTTATTT
GTGAAGCACATTTGGGGGAGAAAATGAGTGTGTGCGGGGGTTGTGACGGGCGGCTGTATATTCATGAAATACCACTTTACTCAGCTC
AGTGACAGGCAGGCCAGCACATTCATTTGTAGTTCTTTTTCTTCTCTTTCTTTCAAAACCCCAAAAGGAGAGCAAGAAAGCAAGGCA
CCTATTCCTCTTTAAATAATGAGGGTGGGGAATTCTGAGACTGTGCTTTTCCCAGGAGCTGTAGGGAGGAAAATTCTAGTGCCTTTT
CTTCGGGTGAGGAAAGTCCGGAGGAGGCGAATGGGTTTTTAAGTGCCCCCTCCCCCGACCCTGTCATTCCACCCCCAGGAGCTCAGC
TGGGATCTGAGGGAAAGGAGGGGAGGAGCAGCTGGGGGCAGGGACCACGCTACTTTCTGGTCACCCAGCACTTCGCTGGGCTGGCCG
GGATGTTGCTGAGTGACAGGAGAGGGGAAGGCAAGGACCATTTGCTGAGTGCTGGGCTGGCTTTACATTACACCCGGGAGTACAAGG
AGCAGGTCGTGCTCACCGCCCCCTGTGTAGATGAAGTCACCGGGCCACACTGACACTGCCAGCGGTGGTGGCACTGAGATAAGAACC
CAAGCCCGTTTAGCTCCCAAGTTCATGCCCTTTCCCTTGCCTGCTGCTGCCCATATCCTTAAAAGAGAAAAATTGAGTTTATGGCAC
TGAGACCCGTGATGCAAAATGTTGGAACAGGAGGAACTTTACCACGAGCTGGGCAGGAAGGGCTTGATGGAGGGCGGGAGATGCTGG
CGCAGGTCCTGGAGAAGACAGTGCTGGAGGGGTGGCCACGGCAAGTGTGGGCACCCCACGTTGGCCTGGGATCAGGGTGAGATTGTG
GACTGGCCCAAGGTCAGTTCTTGATGCCTCCTCTCTGGGGTCTGGTGCTGCTGCCCTCAGGGTGATACCGGGCAGGTTGTGAGGAGG
GTAGGTGTGGAGAATACAGGGAAAATGAAAGCCCTTCCTGCAAAAGCTCCTTCCACAGCCCCTAGCACCCCCACCTTCCACCCCTGG
AGGGCCAGCAGGACCCCTAGACCTGACTGGACATGTCCATACATAACTCTCTCACCCCTCACCTCCACCTTGCTCTTCTCTTAGGCT
GCCTTTCTCCTCCAGCACTGGCTGTGTGGCTGTTTATCGGGGGACAGTCACATGAGCTCAGCATCTTACATCTTACAAGGCATTTTC
ACCCCTGGGTCCCTCATGACCCTCACGGTGGCTGGCCCAGGGCTCATGATGACCAACTATCTCAGGGATGAGAAAACTCGGCTGCAC
AGGGGTTCCCAGGGTTGAGTGACTCACTCCAGGTGCCCATCTCAGGAAAGAGAGGGTTCTAGAATGAGAGCTCAGGTCTCAGTACAC
TTCTGATTTCCCTATTCCAAACCCCACCACAAACTTCTGCTGCGGATTTGAGTGAGGGACCTCGAAGGGCTTCTCCATTCCTGGTTC
TAGAAGTTTCTTTTTCATTCTTGAGTCCCATTTTCCTGCGTCCCTGTCTGTGCCTCATGCCTCTGTTGAGCTCTGTCATGGGCAGTG
AGTGCCGAGAAAAGGCCCGTCTCTGCTTTGAGCCCCTCAGGAGGTGCTTCTGCCTCTTGCTTCCAGCTTGTTGTGTGATCTTTGTCG
AGTCCCTTCCCCTCTCTGGGCCTCGTTTCTCTGGCCTAAAAACAGGAGCTGAACTCCCTTCCTGTGCCGAGGCTTCGCAATCTGATT
CCAGATCATGTTTCATCACGAGGATGAGCAGCCACAGTCCGGAGAGTCCTGTTCTTAGGGGTGTCAGACAGCACCCAGATGGCATCC
ATCCCAGGCGGATGGTGGTTTATGGTGTTGGTGCTTGGGAGGACTGAGGAAGGAGACAGTGACTCAGACTGGGGATGCTGGGTGGAT
TCCCTGTAGGAGGTTGGGGGGTCCCTGAAGGACTAGACAAGCCATGATGGGAGGGGCTCGGGCAGGGGCTCAATTCTGAGTGTGAGA
AGCCCGGGGAGAGGGGGCAGAGTGTGTAGGGAATAGAGCAGGCCTGAGGGCAGCTTCAGGTTGCTGCCGTGTGGTGCACACAGGGGC
ATGTAGGGAGGAAAGAGGAGACTGTCTCGTCTGGAGAGGCTTTGGGCAGGCAGTGAACACAGGCCCAGGGCGAGGGCACAGTGATGG
ATGGAATATTTGGTAGCATCAGTAACCACCAGGGCTGCAGAGTTCTGCACACTCAGGCCCCGAAGACCTGATATCCTTCCTTTTAGA
GGTGGCCAGGCTTGGTAGTTAAGGACTTGGGGCCAGGCGCGGTGGCTCATACCTGTAATCGCAGCACTTTGAGAGGCCAAGGAGGGT
GGATCACCTGAGGTCAGGAGCTCGAGACCAGCCTGGCCAACATGGCGAAACCTCATCTCTACTAAAAATAAGAAAATTAGCCGGACC
TGGTGGCTCACGCCTGTTATCCCAGCTCCTTGGGAGGCTGAGGCTGGAGAATCGCTTGAACCCGGGAGGCGGAGGTTGCAGTGAGCC
GAGATCCCGCCACTGCACTCCAGCCTGGGCAACAGAGTGAGACTCCATCTCAAAAAAAAAAAAAAAAAAAAACTTGGGTGGCACACC
AGTGATGGGCGCTCTGGGAACTTACTGAACCCAAAGCCTATTTCCTCAGTGTTAAGAGGGAATCACAACTACTGGCTGTAGCACAGC
AGCCACCACAGAGCCAGCCACACACTGGGTCTCAGCAGCTGGCAACTGTTGCCTTTATTAGAGTGGTGGTTAGAAGATGCCACCTGG
AGCATCAAGACAGGAGGGGCATATGCGCCCATGTCACCCCTCCACTGTCTGCAGGTGTCCCTGCCTGGAGAGCACGTGTGGAACATG
CCCCTTTTCTCATGAGCCAAATGGCTTCCCGTCCGTGACAAGCTCCCCAGTCATGTTTCTGGATAGACCTTGTTGGGTGAATCTGTG
TGAGTAAAAAGGAAGTGGGCTCTTCTCCGGAGGGCAGGGCTGCAGTGGTGGCCTGGGCTGGACTTCTGGAGCCTTCTTACCCCATGG
GGCCCTGTTATTCTACACTCAGTTAGCAATTTGTGGTCTGGGCAGAGCTAACCGGGTGTGGACATGCAAATCGGACAGGTGGAGAGA
GAACAAAAATAAACCCGTGGGGCCAGGCCAGAGGCTGAGAGGCCTGGAGGAAGGTCAGGGGGACACACGATGACCAGGCAGAAAAAG
GCTGAGAGAGTAGGGCCACAGGGAGAGCCGAACTAGGAAGCTGACCTGGGACAGAAGCAGGGAAGAAAAGCATTGCCACAGCTTGCC
TGATGCGTCGTCAAGAGAACACAATTTCCCCTTCAAGACTTGAGTCAGGGTGCTTTCTAGAATTCTAAAGATTCTTTCTGCCCCTTC
ACCCTGCTTGGAGCACCAGGGCTGTGGCAGTGGGTGACGTTCCCAGCCAGGTGCCAGGGGGCAGGCAGCAAGAGCAGCTGGGCAGGG
GAGGGACGTGCCCAGGGCTTGCCCTTTGTGGAAGCCATATCTGTTTCACAAGCCCTGGGCACAGGGTAACCAGAGTTGTATTCTGAG
CAGAGCCTTGATGATACTAAATGAACAGCATGCAAATGATGTGTGAAATAATATGCAAATTGGCATCTGTTATTTTTGAGTCAGCTT
GTTGGGCATTTTTCTTAAAGGACAGGAGTGCTGCTCAGTGTCAAATGAACAGGAAAGTCAGCTTAAAGGACACTCCTTACAGGGACT
GAGCTGGCACCTACTCCTTAGAGCTTGCTGATACCAGGCCTGCCACGCGACATCTGCAAGGACAGTTGTTTGGTGTTTTGCTTCAGG
TTATAGATGGAGAGACCTATAAAGGTGGGTGCCCTGCCCAGGTTACCCAGTAGGTAGGGAGGAGGAAGCAGATGACCTGTTGCCTGG
CTTGAGCCATTGAACCTTGCTGACCTGTCAGTGGCTTTGAGCTGAGGGGGCCCTGCAGGGAGGCAGGGAGAGGCTGCCCTTGCTCAT
TCTGGTCCATCTGCCACCGTTGGTTTTCCTTAAGCTCTCTGCCTCAAACTCTCCGAGCCTCAGTGTCCCCGTTTGTAATGGATGTGG
GGAGACTGACATCCACTAACCTGGGGAGGATGACACTTGCTAGTGGTTGGTCAAGTGGCCAGCCTGGCTGTGGGTGCTCCTGGGCTG
CCAGCAAGTGGTGGCTCCTCCTAGCCCCTTTGCCTGAGTTGGCATCAGCCTGCCTCCTCGCAAGCTTCCACTAGCAGCGATTTTTGC
AGAGCAAAACCCCTTTTGAGATTTCTGATTTTAACGAATTGGAGTTTAGCTATTTTCTTTCTTTTCTGGCTTCCAGTGGGTAAGATG
TTGGCTCCCCAAAAATCTCCCATACATGGGAATGGCCGGAGCTGCCTGCATGGGTGATGTTACCTTCTCCCAAGGTGAGGGCTTGGG
GTAGGCATGTTGCTCAGGGTTGAGCCACCGCCTTTGGAGGGCTCAGAGCTGGATGGCATTGGTGGAGGATGCAGGGGAGGCCTCCTC
AGGGGCGCACCATACCTGCCCTAGATGGCCAAGCTCTGTAGGCCAGCCAGGTGTGCTAGCCTGGGTGGGCAAGATCCCTGCATATGC
CTCCCAGCCCTGTGAGGAAGGCAGGGAAAGGACCATCCTCCTCCTTATAGGACCCCAAGGCCCATAGAAGTTATGCGGTGGCTCACA
CCTGTAATCCAGCACTTTGGGAGGCTGAGGCGGGAGGATCGGTTGAGTCCAGGACCTCAAGACCAGCCTGGGCAACATAGTGACACC
TTGTCTCAAAAAAAAAAAAAAAAAAGTTACATGACTTGTCCGAGGCTACTCAGCATTGTTAGTGGCAGGGGCATAGCTGGAATCCTG
GAATTCCTGGTTTTAGTTCTTCCTCTCTTTTTGTTTCTCCCATCCCTCTTCTGGAAACATATCCTGAACACCCACTGTGTGGCAAGC
CCTGCTCCAGGCGGCAGCAGTGCAGAGAATCTGTGCCTTACAGACTCCAGGGGGGTCTCTCCCCAGCCAGATGTGTTGTTCTCCACC
AACTCTTCCTAAGAGAGCACAGGCTTTCCCCATAGACCTGGATTCAAATCCTGGCTATTTGGCTGTGTGGCCTTGCGCAGCGGCCCC
TTTATTTGGGAGGTGGTATTTTAATTCCTCTTGTGGGTGGAGAGGTGAAGAATTCAACGTGGGCCAGAGCTGTCACCCAGGGGGCTC
TTGTCCATGCTGGGCAATGCTGGGTTTTACCTTGGTTGTTTCTTCTGAGAGCTCAGTGGCCTTGGGATCATGCAAGGTCCCTCACAG
CTGGGCCCATGGTTCCCAGCCCCTCCTGCTCTGGGCTGGACCTGGTGTTAAGCTCTGCCAAGATGCTTTCCCCTGCTCTGGGAGAGT
AATGGTGCTGAGCCTGGTGGGAGGGCATTTTGGCAGAAGACACCTGACGGGGTGCACACTATGTGCCAGCGATGGAAAGATCCCTTA
AGAGTGAAATGAAATTGATGCAAGCTGAGAGCAAGTCTAGGATGGAGTTGAGTCATGAGGGTTTCCTGGTGGGTGGACGGGATCATA
TAGAAAGAAAGCCTGTGTCCACAGCAGCTTGGGGGAAGGCACCGCTCTCATTACATGGGAACTTATTTTTCCTTCCGTAAGAATGAC
TCCAAAGCAACACCGGGTCTTATTCAGGAGTGAAGAGGTGACAGGCGGTGGAGAAGAGAATATTTATTAAGGGCCTACTATGTGTGT
TTACACACCTCATCTCATTAAGTCCTCACAACAACCCTGTGAGGTAGATATAATTCTCCCCATTTTACAGATGACAGACCCAAGGCT
CCGAGAGTTTAAGTGACTTGCTCAGGGTCTCACAGCTGGGAGGTGGTGGAGTTTGGATTTGCACACAGGGGAGTGTCTCCGACGCCC
TCACATTGTGCAGCAGACATGCTTTTGCATATCAGATCCTAGACTGGGCTCTTTCAAGCCAAAAAGCTTCTCCTGCCCTGCCTGAGC
TCTCCACTCCCGCCCACCCCACCCCTTACTGTGCCACCCTCAGTCCCCTGCCAGCCTCCCCCAGAGCCTCAGGGGATGTTTTGTGCT
CATGGAGTGGGGTAAGGGTACAGGGAGGAGGGGAGGAGGATGCTGGCAGTTTAGATACAGGGGTTTGCTGAAGCTCCAGGTGCCGTG
CTGGGCTCTGGGGCTGAGGAAAGTCTCCTGTGGGCCCTGGAGGGGCCACCCGAGCCCTCAACAGCATGCGTCCTAGTGTTCTTGCCG
CAGCTCTGTGGCTTCTCGGCTGTGCAGCCTTGTTGAGAAAACTGACCTCTTTGAGCCTCAGTCTCCAGGTCTGTGAATGGGGTTAAC
AGTGTTTTCTCTCTCTCATGAGAATGGGTGTCATGAGGAATAAATGAGACAAGGGCACTGCCTGGCGTGTTCTGGTTGGTTCTGGGG
GTTCAGCACCAAATAGACATGGAGCTCACAGGCTGGTGGGGGAGGCAGGCACTGAACAAGTAACAACAGGTCTGATGTGAGCTTTGC
GAGAGACAAGGGCACACACATGTGGCTTGGGTTTGACCCACAAGCCTGAGACTTGGTGTCCCACGCCTCCGTCTGCTCCCCTTCCCC
CCATTTCCTGAAGGTCCATCTCACCCTCACTTCTTCCATCCTCCCTCATATCTGTCTCCCAGGCCATCCTCTGATTCTTCTATATGG
GCTATCTAGCCTGGTGTTAGGAGAACAGGCTTTACTGCCAAGCAGATATTGGGTACAGCGCTGGCTGTGTCACTTACTGTAGGCTGT
GTGGCTTTGGGAAGCTGTCTCAGTGCTTTGATTCACAGTTTCCATGGGGGTGAATCCTAACCTGGCCGCTGGGGCTGTCATGAGAGT
TAATGAGATATTGCTGTTTGTAAGGCAGAGGACATGGTTTCTGGCACACATGGAGCGCTCTGTGAAGGCCAGCTGAGTTGCAGGTGA
GGAGAAGCATCCTAGGCTAGGTGTGGGGCAGCTTAGTTTCTGGTCCTCTGTTTTGTCATCTAGAGAGTGAGAGAAGGAGCCTGAGGA
GGGGATGTTTCCTGCACTGGCCATCTGCGGGAAGGTCGTGAGCACCAGATGGAATTTCGGAGAGAACTGGAAGAAAGCTACGCTATT
CCAGCTGGCGGGGGTCCTCATCGATGGTGCATGTTGGCAGGGTGGGCTGCGGGGGGCGTTATCTTCCTATCCCACAAGCGAAAGCCT
GTGAAGACATCGTGGCTAAGGGGATTTTCTCACTCTCAGGAGACAGCTGAGAGACAGGCCCTTCTGGCCAGCCTCCCTCCCTGCTGC
CTCCTCCCTGCAGGCCAGCCCTTCGCACGAGTCCTGCTTGTCTCCTGAACCCTCCTAGCAGCAGGGATTTAATTACTATAATGTAAG
TAGCTGTGGCTTGAGGCCTTCTGGTGGAGGGGAAACAGATTTGTCTTCTCACTGGGAGAATAAAAGTCTATGGAAAGGAAAAGAAAT
GTACTTTTCCCCGAGGTACCTGAGGCTGTTGGGCTCAGGGGAGCTTCCCTCAGGGCGGGGAGAGGGAGCATGGGTGGCAGCGGGAGG
AGGCACCACGTCTGACTCAAGGACCCTTGGCGCTTGCTTTTGCTCTCTGTGGGATCACTCAGCTAAGCTGCCTCAGTTTCCCCAATG
ACCCTGCTAACAGATCAGCCTGACATTCAGTAGGCATCTGTCAAGCACCTACCATATGCTAGACACTTAGAAACTTCCAGCTGAGTA
GCCAGCCCAGGCGGAAAAATAATTTCATTCATTTGACAAAGTTATTGAATGCCTGTTATGTGCCAGTGTATTGTTCTAGGCATCACA
GATATACAGTCATGCGTTGCAACGACGGGGACGCAGTCTAAGAAATGTGTCATTAGGCAATTTCATCATTGTGTGAGCATCATGCAG
TGCACTTACACAGACTAGGTGGCATGGCCTTCTGCACACGAGGCTGTATGGTGTAGCCTACTGCTCCCAGGCCTGTATTGAATACTG
TAGGCAATCATAACACAATGGCAAGTATGTATCTAAACATAGAAAAGGTACAGTAGAAATACTGTACAATGGATACAAAATACTACA
CCTGTATAGGGCACTTACCATGAATGGAGCTTGCAGGACTGGAGGTTGCTGTGGGTGAGTCAGTGAGTGAGTGGAGAGTGAATGTGA
GGGCCTAGGACATGACTGTAGACTAGTGTAGGCTTTGTAAACACTGTTCACTTAGGCTAACTACATTTATTTAAAATATTTTTTTCT
TCAATAACAAGTTAACCTTACCTTACTATAATTTTATTTATTTATTTATTTATTTATTTATTTTGAGACAGAGTCTCACTCTGTAGC
CCAGGCTGGAGTGCAGTGGCACGATCTCGGCTCACTGCAACTTCAACCTCCCAGGTTCAAAGAATTCTCCTGCCTCAGCCTCCTGAG
TACCTGGGATTATAGGCACGTGCCATCACACCCGGCTAATTTTTGTATTTTTAGTAGAGACAAAGTTTCTCCATGTAGGCTAGGCTA
GTCTCAAACTCCTGACCTCAAGTGATCCACCCGCCTTGGCCTCTCAAAGTGCTGGGATGACAGGCATGAGCCACTGCGCTTGGCCAA
ATTTTTCACTTTATAAGCTTTTTATACTCTTTTGTAATAACACAAACACATTATACAATTGCACGAAAGTATTTTCTTTATATTCTT
ACTCTATAAGTTTTTTTCCATTTTAGAAATATTTATTTAAAAAGATGCATTTTTACTTACAAAATCTTTTTTGGTTAAAAACTGAGA
CACAAACACACGTTAGCCTGGGCTGACACAGGTTCAGGATCATCAAGATGTCACGAAGTGATAGGAATTTTTCAGCTCCATTATAAT
TGTACAGGACCTTGACTGAAATGTCATTATGGGACACATGACTGTAGTAGAAAAAACCCACAAAAGAGAAAAATCTTTGCTGTCACG
AATTTACATTTTAATAGATAAGCCTGATAGTAAACTATCTAAGTTAATTATATATTATGTTCGATGGTGATAAGTGCTATGATGAGT
GTTATGAAGAGCCTCTTCCCTCCAAGATACAACTTCTTACTCCATTGACAAGTCTTAGATCCCAATCTGTTAAGGCCCCAACTAATT
TTACAGCCAGAGTTAGATAACAGGAAAAAGACATCTCTTTTCCTTTCATCATTGTCATCATCAATAACAATCACAATTGTTAGGACC
ACTCTGGATGGGAGTGCAGGGGCTTCTGAGAGGAGAGTGGTCATATTATTTTATTTATTTTCAAATTAATTTATTATTATTATTATT
TTTGAGATGGAGTCTTGCTCTGTCGCCCAGGCTGGAGTTCAGTGGCCCAATCTCGGCTCACTGCAAGCTCCGCCTCCCGGGTTCACG
CTATCCTCCTGCCTCAGCCTCCCGAGTAGCTGGGACCACAGGTGCCTGCCACAACACCTGGCTAATTTTTTGTATTTTTAGTAGAGA
CGGGGTTTCACCATATTAGCCAGGATGGTCTTGATCTCCTGACCTCATGATCTGCCTGCCTTGGCCTCCCGAGATGCTGGGATTATA
GGCATAAGCCACCGCGCCCAGCTTTTTTTTTGTGAGACGGAGTCTCACTCTGTCACCCAGACTGGAGTGCAGTGGCGTGATCTCGGC
TCACTGCAACTTCCGCCTCCCAGATTCAAGTGATTCTCCTGCCTCAGCCTCCTGAGTAGCTGGGATTGCAGGCACATACCACCATGC
CCAGTTAATTTTTGTATTTTTAGTAGATACTGGGTTTCATCATGTTGGTCAGGCTGGTCTTGAACCCCTGACCTCAAGTGATCTGCC
TGCCTTGCCCTCCCAAAGTGCTGGGATTACAGGCATGAGCCACCATGCCCGGCTGAGAGTGGTCATTTTAAATAAATGCTCAGAGCT
AGGTGCAGTGGCTAATGCCTGTAATTCTAGCACTTTGAGAGGCCGAGGCAGGAGGATCGCTTGAGCTCAGGAGTTTGAGACCAGCCT
AGGCCATATAGTGAGACGCCTTCTAAACAAAACAAAAAATTAGCTGGGCATGGTGGTGCGTGCCTGTGGTCCCAGCTACTTGGGAGT
CTGAGGTGGGAGGATCGCTTGAGCCCAGGAGGTCAAAGCTGTAGTGAGCTGTGATCGTGCCACTGCACTCCAGCCTGGGGAATAGAG
TGAGACCTGGTCTCAAACAAAAACAAAAACAAAAACAAAAAACAAAAACCACAAATAAAACAAATAAAGAAGTGCTTAGGGCCAGGC
ATGGTAGCTCACGCCTATTATCCTGGCACTTTGGGAGGCCAAGGCAAGAGGCTTGCTTGAAGCCAGGAGTTTGAGACCAGCCAGGAC
AGCATGGCAAGACTCTCATCTTTACAGAAAAAAAAAAAAAGCTGAGTACATTGGTGTGCACCTGTAGTCCTAGCTACTTGGGTGGCT
GGGGTGGGAAGATCCCGTAAGCCCAGGAATTCGAGGATGCAGTGAGCTGTGATTACGCCACTGTGCTCCAGCCTGGGTGACATGTGA
AACCCTGTCTCTAAAAATAAACACATAAATAAATAAATTAAATAAGTGCTCAGGGAAGGCCTTACCAAAAAGGTGATGTCTATTGGA
GGATCTGAAGTGGTGAGGGATCCAGCCACCTGGGGGAAGAGGAATAGCAAGGTGAAGGCCCTGAGGGGGGATCATGCCTGGTGTGTT
GTAGACAGCACTGTCCGGTGGACACGAAATGGGAGCTACCGCTGTCATTTTAAATTCCTGAGTAGCCATATTAAAAATAGTGAAAAG
AGAAAAGTAGAATTATACATGTTAATAATATTTTTAATTTAACCCAACATAGCCAGAATATTATTATTTGAATGTGCAAGCAATACA
AAAAATTATTGAGATCTTTTACACTTTATTTTTTGTACTAAGTCTTTGAAATCTGGTGTGTCTCTGACACAGCATATCTCAATCAGA
CATTAAGTTTTCATTGGAGATACTTGATCTGTATTTAGAGTTTATAAAATTTACAATTAAAAGAACAGACTTGCTGGCCATGGTGGC
TGACACCTGTAATCCCAGCACTTTGGGAGGCAGAGGCAGGTGGATTGCTTAAGGCCAGGAGTTCGAGTCCAGCCTGGGTAATGTAAC
ACAGTGAGACTCTATCTCTCTAAAAAAAAAAAAAAAAAAAATTAGCTGGGTGTGTGGCATATGCCTGTGGTTCTAGCTACTTGGGAG
GTTGAGGTGGGGGATAGCTTGCTGTTGAGGCTGTGGTGAGCCATGCGTGTGCCTCTGCACTCCAGCCTGGGCAGCAGAGCAAGACCC
TGACTCCCCGTCCGAGAAAAAAAAAAGAATAGATTCACACACTCAAGTTGTTGAAAACGTACTTAAAATTTTTCCAATAAAAGAGTG
GAGTGTCTTTTTATATTTAATTTTAAATGAAATGGACTTAAAATCCAGTTGCACTGGTCATGTTGCAGGGGCTCAGTGGACACACGT
GGCTCGTGTCCTCACACTGGACGGTGCAGGCCTGGGAGCAGAGAGGATGCCTGTGAGGCTGATTCAGTGAGTGGAGGGATCATGTGG
GCGGGGTTGTGAGGTCACAGGGGAGGCGGGTCTGGCTCTGCAGGGATTTGTAGACCGTCCTGAGGAGTGTGGCCTTTACCCTGGGTG
AGATGGGAGCAAGAGGAGGGCTTGGAGTAGAGAAGTGACAAGATCTGACTTACATTTTAATGGGACCACTCTGACTGCAGTGTTTGG
AACAGACAGGGAGGGTCAAGCCAGGGTCAGGGAGCCCCGCTGAGAGGCTGTGGGGACAGCTCAGGTGAGGGTGGTGATGGTTTGGAC
CTGGGTGGTAGCATTGAGTTGGTAAAAAGTGGCTGGACCCTTGGGCTATTTAAAAGGAGGGCATTCCTGATGAATTGGACAAAAGGT
CATGGTGTGCAGGTCAGGCAGTGGTGAGAGAAAGGGGAAGTCAAGGATGAGGCTGAGACTTTTGGCCCAAGCAACTGGAAGGATGGA
ATTTCTGTTAATTGAATGGAGAAGACTGTGGGAGAAGCAGTTTTTTTGGGGAAGACAGGGAGTTTGTGGTTGGACTCGTTAAGTTTG
AGGTGTATCAATTTCCATATAATGCGTGTAGCACCTGCCCCAGGGGTTGTAAGAGGTCACAGGATGGTGCTTGAGGCCGTGGCAAGC
TTCTGCCTGGGCTCCTGACCCAGAACAGATGGCTCCTCTCACCTTCCAGAGGTCTGTTCGAGGCATGGCTGGCAACGTTAAAAAAAA
TTTGGCCGGGCACGGTGGCTCACGCCTGTAATCCCAGCACTTTGGGAGGCCGAGGCGGGTGGATCACGAAGTCAGGAGTTTGAGACC
AACCTGGCCAACATGGTGAAACCCCGTCTCTACAAAAAATACAAAAATTAGCCGGGCATGGTGGTGCACGCCTGTAGTCCCAGCTAC
TCGGGAGGCTGAGGCAGGAGAATCGCTTGAACCCGGGAAGGCGGAGGTTGTGGTGAGCCGAGATAGTGCCATTGCACTCCAGCCTGG
GTGACAGAGCGAGACTCTGCCTCAAACAAAGCAAACAAACAAACAAACAAAAATCAGCTAAGTTATCCATGCCTCCAGCACCGTGGG
TGTTGACTCCCTGAAGGTCACTGGAAGTTGCTAGGGTCTTTCTGAGTCAATCCACATTTCAAGGTCAAATCTCCCTGCTCTGGGCTG
ATGAATAAAATGTGTCATAAATGCTCAGAAGCTCCCCCAGTGTGGCCTGGAAGCCTGATTTTATGGAAACTTACTGAAATGATACAA
AACGCTAAAGATATGCATTCCTAATTTTCTAGCACTTTCAACTCCACAGGGATGTTGGGGGAAGGGATTAATGAGCTTGGAGAGGTG
GAATAACCTCCTCAAGGTCTCCCAGCTGGGGGAAGGTAAGCACTGGATGTTGGGAATCACCAGCCTCATGTGCACCAACCATGTCGC
CAAATGTCCAAAGCCTGCAGAGGGCCCAGGGCTCTCACGGACCACACAGGGAAGCCACTGCACCTGTTCTTTTTAAGACTGGGAGGA
GAGTGGGGAAAGGCAGCAGGTATGGCTTTTGAGGTCCTCTCCTAGCCCAGCTGGTGTCTGATGCTGGCACCCGGGCTTGGAGGGTGA
GAGTCTTGGGACCTGTAGACCAAGGCCACCTCACTTGGGCAGCCCAGGCCTGCTGGACATGGGCCTTCTGACTTAGTTTCAAAGCTG
GACCCTGACCCTTGTTGGTTGCAGGACTTCAGTGATTTAATCTTTGAGTTTATCTGTTTCATTGGGGTGATGGTGCCTGCCTTGGAG
GAGTTTTGTGAAGTGATGTTAATGAAGGAGCTTAGGCCGGTGACTTGACACATGCCCAACATAAGGAATGCCTGTGGGGCCGCCTGG
CTCTGCCCCTAGAGTGGAGGGAGATGGTGCAGAGGGAACAGTAGAACTGGTATGGGCTGCGGATGCCTGGCGAGTGGCATGAGTAGG
GGAGAAAGAAGCGCCTGCCAGAATGGGAGTGAGGGAGAACAGTGGGGGGTCCCCTCCCTTACCACAAGACCTGGGATCTAACTGTGC
CTTTAGATCAGAGCTTCTCAACTGGGGCACTTTTGCTCCTCAGGGGACTTTTAGCAATGCCTGGAGACATTTTTGCTTGTCACAGTC
AGGAGAGGGATATTATAGTCCTTTAATGTGGAGAGGCCAGGGATGCCCCCTACAACAAAGAATTACCCAGCCCACAATGTCAGTGGT
ACTGAGGCTGAGAAACCCTGCTTTCGATGACTCATTCTCACCAGCTTGTTATGAGGATGAATGCGAAAATGGAGCCTGCATTTCTAA
CTGCAGCCTGCCCTTCCAGGTCCTCTCGTTCCCTCACCCTTTCCCAAGGGGGCTGCCTCTGTCATCAAGCAGCCTGGTCCAGGAGTC
AGCAGACCTGAATTCAAGTCCTGACTCCTCATGCCTCAGTGAAGTTTTTCCTTAGTTTCTAGTCTCCATGTCTTTTCATCCTTGGAA
ATCTTGTCTTGAGTGCGGCAGTGGTGTCTACACAGTAATGGGGCTGTGGTGTGGACTGAGTGGGAACCCGGATGGGAAGCATGCTTA
GTCAGCTGTGATGTGCCGGCAAAGGGAGGCAGCAGGCCAATCCGCTTTCTAGTCCAGCACTGAATAATAGCCTTGGGGGGCATTGCC
CAGGAACCCCCATTGGCCCCTGCGAGGCCACATGGCTGTTCTCCCAGGCCTGCTCCATCTTGCTTCTTGCTTGTGTTTCCTTGAACA
GGTGGAGCTAGTCTCCATTTGGGGAAGATGGAATTTGCAGGAAAATGCAGGGCTATTATTTCTGTGCTGTGTGAGTGGGAGACAGAG
CTGGTTGAGTGGCAGATTTCCTGGCAGGGGGAGAAACAGCTAAGTGTGCCAGCCCAGGCATCGCTAGAATCCTCTTGAGGTGAGTGG
GCCAGGATTCCCCAGTGGCCACCTCGGGAAGCAAACATTTGTTCTGAGCTCAGGACCAGCCCCAGGAAAAGATGGGGTTTGCTGTCG
GGGAAAGGTCTGTACCAATATATTGAAGCTCTGAGAACATTCTACTCGGCAAACTTTCACTGATGCCCATTCTGTGGCAGGCACTGG
CCCCGGGGACGTTGGGGGACACAGAGATGGATGAGGCTGAGGGGGTGTTCGGGCTGCCGTAACTCTGAGGCAAGGCAGACTGTGCTA
AGTGCGGTAGAGAGGCTCTGCATCCCGTTAAGGGCCTGCCGCGGATATTAATGTTTGGAGGCTTCTTAGAAGAAGTAGGGGTTGAGC
TGGGTTTTGAAGGAAGGTAGGGCTTCATGAGGCAAAAGGGAATGCGTGTTCTGAGCCGAGGCAACAGTAGGGGCAGAGGCTCAAAGT
TGGAGAGTGCCATGTGTTCTTAGGGAGGGCTGGAGAGGTAGGTTTTCCTAGAAGTTCTGGGAAGAGAAGACTGGAGAGGGACATGGA
GCCCCCTGCCCGTGAAGCTGGTGGAGTAGTGGGGGCTTCACGAACTAGGCTGATGAATTTGCACTTTTGTCTAAAGGCACAGGGAAC
AGTGGAAGGTATGTGAGCAGGGCAGTGGTGTGCTCCAGCCACAGCATGGAGGATGGGTTATAGAGAGAGGAAGCTGGAAGCAGTGTT
ACTGCGATAGTTGTGAAAAGGAGGAATGAAGGCTTGAACCCAGCTCGGGGCAGTGGGAAGGGAAGATATGGGGGGAGGTCTGGGGAG
GCAGGCAGCTTCTCAGGCTTCACCTTTCTCCATCCTGGGTATCTGAGTTGTGTGTGAGGCTCGAAGTGACTGTCGTGGCCCACTCGC
CACATTGCTCTGCTCAAAGCCCAGCCTTTTCTTGGAGAATGACAAGCAGCGAGCATCAGGCAGCCTTCTGGTCCCAGAGAACAGGAT
GGCACGGTGCCCACACCAGCCGGCACCAGCCACTCTGGCTGGGGAGGGGGGGAGGCCTCCTTCTGGAACTCATCAGCGCTGGCTTCT
GGCCAGGCCCAAAGGAGGTGGAATACACCCTCTTCCTGCTCGTTTTCAGAGATTTGGGAGGAGTAGGTATGGAGGACAGCAGCAGCG
TGCTGCAGCCGGCTACTCTCGGGTTCGTGTCGGAGCTGGGGGAGTGACTGGCTGTGCAGTTGGGCAAGTTGCCTGGTCTCCCTGAAC
TTCAGTGTCTGTGTTTGTAAAGTGGGCATTATGATGCTTGGCCCACGGGTGGCTGTAAGGGCATCACACAGGTGTAATTACGCAGGT
GTCATGTTCTTAGCTCAATAAATGGCCGCTGTTTTTAATTGTCAAGATATGATGATGGACTTGACTTGATGGGTGGGGAGAGGCAAG
ACCTCCTTGCCTTTAAAGGAGTTGAAGACTTGGGAAGCAGTTGGAATGGTGAATTCTATCTTGGGCGTGTTGGCTTTGAGGGGCTGG
GGAAAATCCTAGGATATTCAGAAGAAATCTAGGGTCTTCTAATCATGTGGTTCACATAAATGACCCTTACTATAATATCTATCTCAG
AGTCAACTTTTCAAGACACCAGCCTCCCTGCAGGCAACCCCAGTGTGGCATCAGGAGAGCGGGGTCTTGGGTCAGCATGGCCAGCCC
AGCCTCTGGCTTTGCTTGTGTCATTGGTGACTCCCCGTGGTCCTCGGTGCCTTCCTCCGTGATGGCATTTCCCATTGGCTGGCTGGG
CCTCTGCCCAGGGATCCAGCTGGGTGCCGGCCCAGCTCAGGCTCCAGAGAGGAAAGAGCCCTGGATCTGGGGCCAGTGACCTGATAT
TCACGCTGGCTCTGCCCCTTCCCTCTCGGTGTGGCCTGGAGAGATGTGCAGCCTCCCCGAGGGCAGGGGGGGGTAGTGACGAGGGTT
CCAGGAGATGAGGGATGTGAAAGCAGCAGCACGTGCCTGGCACACAGGGGCCCTGGTAGATGCTTGGCGGACAAGTGAGGGCAGGCA
GGGGAGGGGGCAGACAGGATTCAGGGAGGCAGGCCTGGGAGGAGCCCAGGGCTCAGGGAGGGGGGTGAGGGGTCAGGCGGAGCCGGC
TCAACTCCTGGCTCTGCCGCTTACTAGCAGGAAGACTCCGAGTCCGGTGCCTGCTTTGGTCACATCCATGTGTGGGTGGTGGCGATG
GGAGGTTGGCCTTGGGGGCCGCTCTAATGCCTACCTCACAACATAGTGATAGGTCTAGATGGATACACAGCACCAGGGCCTGAGAGT
GCTGAATAAACCTTCATTCCTCCTCTAGTAAGCACAGTAAATCCCTAGAGGGCAGTCCCCAAAGGCCTTGATTACCCCAACTGTGGA
GTACAGCGAGAAGTGGAGCTTCTGGAGCCAGTGCTTTCTCTGCTCAATTTTGTGTATTCTTTCACTCGTTCATCTGAAATCTGACCC
AGCCCCATGGAAACTGGCTGCAGACACCCAATGGCAGTAGGGTACCAAGGGGCAGGGGAGCAATCGGCTCCCGGTGCAGACAATGAG
GGGTACACTGTCTGTAGAGGATATAGAAAAATAAATAAAGCCAACAAAAAGTCTGGTTTTTGTTATTGCCGTGTACCGGCACTTCTA
AACAAGGTCAGTGATAAAACACTCCTCCTCCCGGGGGGCAGACGCTCCTGGCTCCCCCTCAGTATGCTGCTGCCTAATCGTCCTCCA
GAAAGGAGCCTCGGCGGCTGTAAATATCAGGAAGGAACTCAGCAAGGCCTGGCAGAAGAAGCCAGCGATGATTTGGAGAATAAGCCC
ATTGATGATTGCAATAGCAGCATGGATTTGGGGCTCAGGAGACCAGGAAGGGGAGGGTGCCACACTGTGAAGTAAGGGTAAGGCAAG
AGAGTGGAGGTAACTGCTGTGGGTTCTGGCTGGGAGAGGCATCGTCTAGGCCCTTTAGACGTGGTCAGGAAGAGGGTGTACTTCATC
TCCCTTGGGCCTGGCAGGAAGCCAGCGCTGATGGGTTCCAGAAGGAAAGCCCCCCAGCCAGGGCAGCCGCTCCCAGCTGGTGTTGGC
TGGTGTGGGTGCTGTGCCACCCCACTCTTTTGGACCGGCAGGCTGCTTGCCATCTTCCAAAGGGGACTGGCCTCTTGACAAGGGTAG
GAAAGGAGCCAGGGCTGCCCTATGGGCTTGGGCGCTGGTAGGGCGTTGGGGAAGGCAGGATTCACATTTCTCTAGCCTGGACAACAA
GCATGTGCCTGAGGATCAAAATGAGCACTTTTAGGGTTTTATTACTATTATTTACTAAACAAGTTTCCTTGCAGGGTTGTGGAAGGG
GCCCATGCAGAAGGATCTGTCTAGAGGAGGAGGCTCCGCATGTGTACAAGGGAAGGACGTGTGCCCTTGGGACTGGCTGGGGCACGG
CCCTAGGGAAGTGTGTGTGTGTGTTTGCGGGGGTGCTCTGGTCATGTGGCCCCTTCCCTGACTGAACCCCTTGCAGGGCCCCATGCC
ACACAGCAATGCTGCGTCCGTTGCAGAAGCAGACACCTTTGGCTCCTGCTGTGTGGGAAGCAGGTGTGCATTAGTCAAGGTGGCCTC
AGTGGTCCTGGGTAGTGGGGCCCCCGCAGTGAGAAAAGCAGAGACCTCTTGGGCCCGGGGGTGTAGGAGTCACTCTCCCTGATCTGC
TCCCAAATGCAGGTGCATCTGGTTCCATTTGGAGCCGGTGAGCTGGCTGATGGTGGCTGTCTGGGGCTCCTGGGGCTGAGCAGCCAG
GCTAAGCTTAGCCCACAGGCACTTCCCAGAAACTGGTGGCACTGTCACAGTGTTGGCTGGATGCTGAGGAGTTGGCAGGATACAGTG
CAGGAACAGCAAATAGGTTTTATCTGGGTGTCAAGTTGAAACAATTGGTACCAGCTTCCAGGAGGACTGTGTTGGGAAGGATTCTGA
GGTGTTGTCTGAGCTCAATGGGAAGAGTGCCCTGATTGATTCATGAGGTCTATCTTGGGCAGGGGAAATGGAAGAGTGGCAAGGGGT
TTGCCGTCCCTGGTGTAGTGGAAAAAATACAGAGAGCCTAGATTTGATCTTAGGTGAGTGGCCCCAGTTTTTTTTCTCTCTCTTTTT
TTTTGTTTTTTGAGACGGGGTCTGGCTGTGTTGCCTAGGCTGGAGGGCAGTGGCACAATTTCAGTTCACTGCGACCTCCACCTCCTG
GGCTCAAGCCATCTTCCCACCTCAGCTTCCCAAGTAGCTGAGACTACAGGTGCCCGCCACCATGCCCAGCTAATTTTTGTGTTTTTA
GTAGAGGCGGGGTTTCACTATGTTGGTCAGGCTGGTCTTGAACTCCTGACCTCAGGTGATCCACCCGCCTCAGCCTCCCAAAGTGCT
GGGATTACAGGCATGAGCCACTGCAACTGGCCCCCTAGTTTCCTCATGTCTAAAATGAGGGTAAAAATGTGGCCCTTCCAAGCCTTA
TTGTGGGCGCTGAGTAAGATAGCAGAGCGAAAGCCCTTTGTAAAATATAAAAAAGCGATACAAACCCAGAGTGGGGGTTTTGTTGTT
ACTGAGTCTCCCAGGAACCTGGAGTGACTTCCCAATAATAACATCTGCCCCACTGGGGACTACTCCATGGTCTGAGTGCCTGAGAGG
CCTGACAGGATGTGTGACCGGCGCGGTGCCCGGCACCCAGGCCTTTGACACCAAGTGTCAACGAGGTGTTTGGTCATGTTTCCAACT
CTAAGTCACTTCATTTCTGTCTGACTCTCTCCCTGGCTTAAGATTTAGAGACTGTTTTCGTGTCTAATGAAAGAAGCACAGAAAATC
TCTCTCTAGAACCTTGTCTCTTACTCTCCATTAAAGGTAGCAAAATTGCTTTATAAAGTCTAAAAGCCTGACAAGAACGCGTACTAA
TAATGCATGAATTATGCAAATCTGTGTAGTCACTTGATTTGCATAATGGAGGGGCTGGAGGAGGGTTGGGGAGGGGAAGGAGTGAGG
GTGAGGGCGTCTGCCCAGATCTGTGTGTAACGGGGGACATTCATTTGCTCCCTTGGGTGGAGTCCCCTTCTGCAAGCCCCTGCTGGT
CCCGAGGGTGTGTTCTTTGGCCTTCCCCTGCTGGCCTGGTGTCTGCCCTTTGTGGCCTGGCTGTGCAGCCCCCCCTCCTACTTTCGG
GGCAGTCACCGAGGCAGGGGCACCGCACCCTGTGCTGGACTCTTGTGTAGAACTGGCTTCATTAAATCTCACAGCCGCCCCTCCATG
TCCAGACAGCCTTCCCTGAAAGTCCCCTGGGTCATTATTTCTCCAAACGTCTGTCTTGCTGAGGCATTGGACTTCGTTAAGTGCCGT
CTGGAATTGTAACTATTGCCAAGTGCCTTTTTCTTGGGTTCTGTGGGAAGCAAGGGTGAGAAGGAGTCTCCGGGAGAGAACTCCCCA
CCTACCCCGCCTGGTTTCTCTCTCAGCGTTTCTCACTGTTGCTTTAAAAGGTTTCAGCCTCTGCTGTAAGGGGCCTCTTGCTTTTCT
CTCCCAGCCTCCCTGCATACTCGCTAACCTTGGGACTTCAGATTTGATGAGGGGCCGGGCGCAGTGGCTCACACTTGTAATCCCTGC
ACTTTGGGAGGCCGAGGTGGGAGACTTGCTTGAGGCCAGGACAACATAGCCAGACCCCATCTCTATTAAAAAAAATATTTGATGAGG
GCTGAGGCTGGGACTGAGGATACTTGAGGCTTGAGCTCAGTGGGTGCTGCCTGGAAGCCCGGGGATGTGGGCTCTCAAGCTGAAGGG
ACGCTGGGAACCAGCTGTGGAGAGCCAGGCAGCTGGCTTTTTTGTCTAGAACCTGGATGGGGCAGCTTCTTGTTCTGGGGAGGGGAA
CTGTTCTGCAGGGTGGATGGATGCAGGGAGCCAGCCCTGGTTGGGAGGTGGGAGGGGCAGGGACCATATCTAGCTCCTGGAGAGGCC
AAGCAGACCGTCAAGGCTACAAACAGGCTGGGGTTCAGCTTATTTCAACATACTTCTTCAGCATCTATGTCTATGGGCTGGGCTTGT
GCTAGGCCCTAGGTGTCAGAGATATGAAGGCACTCATAGCTGTCATTTATTGAGCTCCTATTATAAGTGTCAGGTCCTTCGCTAAGC
CACTTATTTACACGTGTCATCTCATGTAATGTGTATGACAACTTTATGAGGTCACTACTCTTACTGCCCCATTTTATAGATGAGTAA
GTTGAGGCTTGGTCACATGGCGAGGAAGTGGCAAAGTGGGGCTCTAAGTGAGGTCTTCGGACTCCAAAGCCCCTTCTGCAGACCGTG
ATGCTTTGCTGCCTCTGTTGAGTCAAACCGGACTCTTCCCTTGAGGCCCTGATGGTAACAGCAGTGTAAACAGATGGTGAGCACAGT
GTAGTGTAATATATGCAAGGGGAGAGGTTGGAACCAGGCACAGAAGTAGCACTGAGCATTTAATTATGTCAGGGAGGGCCGGGGAAA
GCTCACAGAGGAGGTGGTGGGGGGGGGGGTCCCTGAATGAGCCACAGGAGGGAAACCAGATTCTGTCTGGCACTTAGGGAAGCAGCC
AGTGGAGGCTGCAGGTCAGTGGGCTGGGGTGAGGGGTAGGGGCTTTAAATCCCAGTTGCTTATAGCCCCCTTCTGCCCCACTTTTGG
GGTGCGACCAGGTGATGTGAAGCTCAGGAGGTCATGGCTGGGACACTCAGGAAGGTCAAGTGTGGGGTGCTGCCCCACGGCCCCTCT
CCTCGGCCTGTATGTTCAATAGGTGCACTGGCCTCCCTGTCTTGGCCAGACAGCTCTGAGAGCCAGGGCTCTGTGGGTGCCTGAACT
GTGTGTGGACCCTGCCTCGGATGAGGCCACAGCTGCTCCCACACCCGCACCTGCTCACACCCAAGAAGGGGGCCGCCATGATCACTG
TGGAGGCCAGGGTGGGAGAACCCTCTGGACCATAGGGTCCCACCCCATTGCAGGGCAAAGAGGGGATCTGAGGCCCAGTGACTGGAA
GTGATTTGCTGCAAGTCACACAGGCAGCAAGAAGTGGGTCAATCCCCCAAGGTTCTGGTTTGGAGCACTCCCGGGGGTCTCCCCTCA
CCCCTTACCCTGGGCTCGCCCTCTCCAGGCTGTGTGCCATCCTCACCACCTCTGTCGTGCCCTCGCTCACCCACGTGGCTACACTCT
CGCGTGCGTTTCCCTCACACCTGGCCATCCTCTTAGGCCAGGCCAAGGCTTTGCTCTGGAGAGAACTTAAATGGCATTGTCAGATGA
GGAAGGAGCAGAACCTGCCGATCCCATGACCTCCCAATAGGCCATCTGCACACACTCAGCCCGCTCTCCCCAGCCCTGCACAGCCCC
TTCGGCTCGCACATCTCATCCTTACTCCTGCTAGCAAGCAAAGATGTCTGCAGGTGCTCTCATTCGGGGATGGGGATGTAGTAGGGA
GACAATACCTGGTAGATAGAGCTGAACAACTTTCTGCAGAAAGTTTGGAGTGAGGGGGGCTCTGTGATGGAATAACAGGCATGAACC
AGAAGCCTAATTCTATTGCTTAGAAGCTGTGTGGCTTTAGGCAAGCGACTTAACCTCTCTGAGTTTCAATCTCCTTATTAATAAAAC
AGGTACGAACAGTGCCTTGCTCAGTGGTCCCCTAACTTGGCTTCATTTTGGAATCCCCTGGGGATCTTTAAAAAAATGCTGATGCCT
GGCTCCCAGCTTCTGACCTGATGTCACCGCCCTGAGGTGCAACCTGGGTGTGGGGATTTTAAAAAGCTCCCTGGGTGATACTAATGT
GCCGCAAAGTTTGGGAACCACTGTCCCAGGGTTTGTTGGGGATTAAAGGAAGAATGCAGACACGTGGAGAGCGCTTACTAAATGGAG
GTGATTATTTCAGTGTTCATCTCCCTGCCTCTCACTCCATTTAATGTGGAGTCTGTGGGACTGAGGTTTTTATTTTTATACCACAGT
GGGTTTGCCAGAGTGCTGACCTAAGGGGGCTGTCAGCGGCCCATCTTTACCCGGAAAAGCCCCTCCCTTGCGCCCTCACCATCTCTG
CAGCCCTAGGGCAGGCCTCAGTCTTTCTGACAGGCCCCAGGGTCCTGATTAGGCCGGGGCCTGACCCCGCAGGGGCAGGCCTGCCCC
TGGTCTCCACCTGGTGGCTTTGGGAATGGAAATGCTGGTTCCCACAAAGGCTTCTGGGAGACAGAAGCCTAAGATGGCAGGGTGCGG
TGCACACACTCAGTCTGGATTCGGTGGCCTGACCTGGGGGATCCGGGGAGGGCGTTGGTGGGGGCAGAGGCGTGGGAGGGGCTGCTT
CTTTGGAGCTCTCCACTCTGCTCCCTGGTACTGTCTGAGGCCCTGCAAACCTGAGCAGAGCCTGTGAGGGAAGACACATTTCTCCAG
ATTCCTCAGCTGTCCTGACAGGAAGTTGTGCATCACTGGGCTGACAGGAAGGAAATGGCTCTGCTGTTTGGAAAGAAATAGAGAATT
CTGACTGGGGCTGAGGGAAAAGGTGTCTGCTTGGAGAAGCCGTTTTGTCCCGATATGAGGACTCTTTGTCTCTGCAGGCAGTTGCTC
CCTGTTTATTTTCCTTTGAATGACAAGGATTGGCTTCAGTTAATGAGGAGCGGTGAGAGGAAAGTGAGAAGCGGCTTGTGAGATTTT
GGGACACGGAAGCCTTCGCAGGAAACCTGGCCCTCTGCTTGTAATAAGCATCTTTCTTTCCCAGCTCCCTGCTTCGGGGACTGTGAG
GTGACAAAGGATTTTGTGATCTCACACGCCGCTTTTTCTCCATCCACCAACCATACCTTTGCAAATGCTTTGATTTCTATTTCTTTT
TACCACCCCAAAAACACAGTGGATCTTTTAATAGCACAAGGCCTTTTGAATCTCTAACCAATCAGTCTCCAAATCCTATCCAAAGTG
CACTTAGAATAGACACTTTTAATAAAATATATTGCTTTTAAGCCAGTGTAAACCTGTGAAATTTGTACATTTCTTTTAGAGCATCTC
TACAAATTTTACATTATTGAAATCACCACCCAGTGACCTTTTAGGAAATACTAGATAAATTGCAAGAGACAAATAGACAAACCAAAT
GGAGTAAGTACAATTTCCAGTGCCTTTCCTGCTAGGTTCCCAGTCTGAGTCTTAGAAATCCCTGACTCCCTGTGCCCCTCCCCCCAG
CTCTGTGACCTAGCCCCTGCTCTGCAGGCCTCTCTGCCCACCCTGCCCCTGGCAAGGAGCTAGCAGCTGGGGTGTCATCTGAGAGCC
ATTTGCAGCTCTGAGAAGCAGCTCTGGTATGTGGCTCCTGGCACCTTGTCCCGTCTGTGTTTGTCTCTGCGCTCCATCATTCAAAAC
ACTTCCACCAGTGGCCAGGGCCCCCAGAAGGGCTCTTGGGGGTGGCTGAGGACTGCTTTAGAAAGGCTCAGATGCTCTGTGGCCGAG
TCTGCCCCAGGCGAGGTCCTCAGCCTTGTCGAGTGAGAGCTGAGCGGGGGTCTGAGTCCAGCAACCCTCACTCACTGTGGAGCACTC
TCTGTGCCTGGGTCAGCCAGGTGTGGATTATCCACTTTGTGGATTGCCTGTAGTGTCTTTTCCAAACCCAGGTCACGTTCGTCCCTG
CTGCTGTCTCACTGCATGGGGACCTTTCCTCCCTATCAGCTGGTGGTGCTTAGGGGATGTAAATCAGGTGTCGTATCTTGTTTGTAT
TTTGTACAGGGAAAACAGAATTTGGAAGACAGATGGTCTCTAAAATCACATTACCAGAGCCTCCAAAGGGTCTCCTGGAGGCTCAGT
GCTCCTTTGGGCTATGGGATCTGTTCCCCTTCTGGTGTAGGTACGCAGAGCCTTGGAGCAGTGATGGGGGTGGAGGGAGCACAACTG
CGCCCTGCTTCCTGAGGTCTGCAGATCGCAGGTCACTCGATGGTGGCGGGCACGTGGTTATGCACTCATTTCATGGATAAGTGAACT
CAGTCACCATCTCAGCTGCTTTGCCATCGCTACACAGCGACCTGTGTCAGCACCACAACTGGGAGTGTTACTTGGGTCCCCTGTATC
TCAGTTGCTGTTTTTCCCATGGAGTGACCCGACTGCCCTCCAACATGGGGAGGGAGGGAGAGCAGAAGGCAGATCAGCGGTTAGAGA
TGTCCTGAGTGGCACTGGCCCTTCTGTAGGTAAAGTAGCTTCGGGAGAAACAAGGTACTGTCAAAGCTGCCCAGGCTTCATTCACTC
CTTAATTTGATAATTAATTCCACAAACATTAATGGAGTTTTTCTCACATTCCAGGCACTATGCAAGGTGCTGGGGGTGGGGGAGAAA
CAGAAAGATGACAAAGACTTGGTTCCCACACTCAAAGAGCTCACAGCCTGGTAAGCAGATGCTCAACGCACTTAGAATTGTCTCCCG
AAGAGGCCTGAAGGGACAGGGTGGAGGAAGCGCAGAGGCGGAGGCAGCTCTGGAGGGTAGGGTGGTGGGAGTGGCGAGAACGGAGCC
GGTCCTGAAGGTTGACTGGGAACGGGAAAGGCTAAGGCAGGGAGGTTGTTCCCGGCCCAGCAAGCACATGGAAAGGTTGAGGGAAAC
GAAAGCTTAGTGCATTTTGAAGGAGTGTGAGCAACTCGGTATGGCTAGAATTTATGGTGTGAGTATGAGATATGGGGCTGTAGAGGC
AGGCAGGGGTCAGATCATGCCTTGCTTAGGGGGTCTGCCTTCATCCCAGAGGCTGTGGGGAGCCGCAGAAGGGATTTAAGGAGTGGG
ATGGCACGATTTGTGTCTTAAATTACTTGCTATGGAATCTACATGGTATAGCAGAAGGAGCCCGGGAGTAGGGGAAGGAGCTAGGCT
CTAGCTCTAAATTCTAGATCTGGATCTAGCAGCTGGGTGACTTTGGCCAAGTCGAGGCCTCAGGGTCCTGACCTGTAAGATGCAAAG
ACTCTAAGGTCTGTCTTAGCCCTGACGTTCTCCACACCCGTGGCCGCCTTCCTGTTTGGCCCCCACACTCTCTGTTCTGTACTTGTC
CATCCACCTCTGGCCGCCTGACCCCTGTCAGCTGGCTGTCCCACCGTGGTCAGCAGCTGGTGGCAGCTGGAGGGAGAGGGGTGAGTC
CTCCAGGAAGCATGTGCACTCCTGCCCTCGTAATTCAGGCCTGCCTGGCTTCTCCTCTTCCAGCCCTGCGAGAGCGCCTTCCTCCCC
GCTGACCTCAGCACACCTGGCTCCCCACGGCACACAGCCCTGGCTGGGCCCAGCTGTGTCTCTTCAGTGAGACAGTTCACCCCCTTG
GGCCAAAATCCCCACCCACCTGGCCCCCACGTGACAAGAGGCAGCTGCTTCGGGGTTGCCACCTGGTAGCCCGTGACATCTGTGAGA
TGTCTGTGCATTTGGCTGGTGTGAGCTTTTTCCTTTGCTCTGACCCAAGCTTTGATAGCTCAGTGTGTCATCACCGGCAGTGGATGC
CTGGGAGCCCCAGACTGGCTCTTCCTGCCCAGGGACCCCCAGACCCACTTCTGCAGGACGCCTTGAAACAGCTGTGGGTGGAAAGCC
AGGTTTTCCCCCATAAGCATCACTCGTTCTGCTGCCTTCCTAGCTTCTGTGCTCTCGGGCCCAGAGCTCCTCCAGGGTGAGGAACCA
GGAGGAGGAGTTGGCTGGGTTGGCTGTTTTCCTAGGTCTTTGATCACAGCTGTTACATGTAGATAGGCTGGGCGCGGTGGCTCATGC
CTGTAATCCCAGCACATTGGGAGGCCGAGGCGGGTGGATTACGAGCTCAGGAGATCGAGACCATCCTGGCCAACATGGTCAAACTCT
GTTTCTACTAAAATGCAAAAAAAAGAAAAACACAAAAAATGAAAAACAAACAAACAAACAAAAAACTAGCCGGGCATGGTGGTGGCG
TGCGCCTGTAATCCCAGCTATTCAGGAGGCTGAGGCAGGGGAATTGCTTGAATCTGGGAGGCAGAGATTGCAGTGAGCTGAGATTGT
GCCACTGCACTCCAGCCCAGCAACAGAGCAAGACTCTGTCTCAAAAAAAAAAAAAGGCTTAAACAATGGGTTCTAAGCAAATAAGTA
TCTGTTTAGTCAGTGGAACGAGGAAACAAAAGTTGATGGAGTTCTCCCCACAATTGGAAGTGTTGTTCCCCACCTAGATGATGGGGC
CTGCAGGGAAGTGATTTTGCACGATTCTCCTCTTCTTGGGAACAGGCCATAGGGTAGCATCCCTTCATTCATTCATCAAGTATTTAT
TGAGTGCCTACTGTGTGCCAAGTCCTCTGTGAGGTGATGGAGCAGAGAGACAGAAAGGACCCCGACTGTGCCATGAAGCAGCCCCGA
GCCTGGAGGGGCCACTCAGAGGGAGCCCAGGGGCGGCGGCTCTCGGGCTCCAGCCGAGTTTCCTTCAGACCTAGCCCCTTTAGCTGA
CAATCTTAAAGCCTGGTCCCGGAAAACAGGAATAGTGCGAGAGGGATGAAATGTGGGTGAGGAGAGTGAGACAAGGGTGGAGGTAAG
AAAGAGGGATGGAAGAAAAGACGCAGCAAACTTTCCCGTGAGAGAAACGCTCCAGACAGCACCCCAGTTCCCAGCTGAGACTCCCAG
CCACAGTCAGCAAGAACCCACAGGAGGGGAGGCCCACGTCCCTGCTCAGCTCTGCCCTGGCAAGTGGGGTCGGAGATGGGGCATTCC
CTGCTTGGGGTCTCAGTCTCTCCATCTGTAAAACGAGGCTTTGGACTAGGGGAGCTCAGAGATCTGTTTAGAACTAATATTCTGTAA
CCGGTGCAGGGCAAAATAGACGGGGGGCAGGAGCCCAAATTAAGGCCAGTCGAGTATCAGGTGGGGTGTTCCTCTGGCTCCTCCTAT
TACAACAGCAAGAGGGACCCAGGGGTACTGTGGGAGTCCTACCATCTCCTCACTTGTACGAGGTACCTTCTGGCTGCTCTTCCGGCT
GCTCAACACTCAAAGAAGGAAGGTGCTGGGTGTCCTTGTTTGCATATTTTTCTGGCACGGACACATTCTTCTTGGAGTTTCTTGACA
TTAGGGCCACTTGAAATGCTCAAGGTAAGGGCTGCTCTGCCTGGCAGACATGCGTCAGGTGCCTTTGGCTCCTCCCTGGCAAGTCTG
CCTCCAGACGCCCTCAGATCCTGTCCCCATGCCTGGCCATGGGGCTATCTGACCACTGATGCCAGAGGGTCACACTCATCAGTCTAT
ACAGAAGACTTGGAAATGTTTTGGCAACCCTGTGTTAGAAGAGTGGGGTTTCCTAACTGTATGGCTTTGGGTAATTGCTTAAATCCT
CCCAGCCTTAGTTTTTCCATCTGTGGGTTAGGAGTGGTCCTCTCTACCTCCCAGCATCAAATAGGAAACTGTGCAAGAAAATGCTTT
GTAAACAGGAAAGCCCTAAACTGACATCCAGGACACAGTGACTCAGTAGCAAATATATTCTGATATTCAGCCTTTTCCATGGTTACA
GCCTCCTCTGTGCCAAGAAATTCAAGTCACCAAGGGTTAAAGCAAGCAAGGCACATGGCCGTGATTGTCCATAATTGATGAAATAAA
CCTTAAAGGAGTGACATATAATTAATTGCAATTAATCCAGGCACTGTGACACCACCTTGCTAAAAATACTGTCTACTAACAATTGCT
TTCATCCAGCAGAATGATGTCTGCCCATGAGCCTGGCAAAGTGTCTGCTAATGGAGTGCACATAGTGGGTGTGGAAGCTGGCAGGTG
TGGGGGCAGAGGGCGGGAAGGGGGCCCCAATTCTCTCAGACCCTTTGGCAGCAAATACTACATATATAGGGTGATCATATGATTCAT
TGTCTAAACTGGGACACTTTTGAGATTATTATATTAATGATTACCATTCATAGTAATAATTATGCCAGATAACAGGCATACACTGGA
CTCTCCTGGGTAAACCCGAATGTATCGCCACCCTCTGCATAGGCTGTTTTAAGCCCGTGGGAACCCTCGACTGGGAGAGTATTTTCA
CGTTCATTATCTGCTCTTCCTCACAACAACCCTGGAAGGAAGAAAGGATTTTTTAGTCTCATTGAATAGATGTTAAAGATGGTGATG
GTGTCAAACGGTCCAGCTCTAGTCTTTGGCCTGGCTTCCTCGGTGGCTGACCCCATGCAGGTAGAGGACAGCTGGTCAGGACCGGGA
GTTCTGGGGATGTGGAAACAAGTGCAGCTCAGGGGGAAAGAAGTAGCAAATCTGGCTTCAGTGGAGCAGGAACAAGCGACCTCCTCC
GTGAAGAGCTCAGGGTGTAGGTCTCAGCCGGGACTGTGCTGCGGTCCTTACCAATCCTGGGCTTGGAATCAGAAGCTGTGTGTTTCA
AGTCTGGCTCCCACAGTTCAGCTGTGTGATCTTGGCAAATTGTATAATCATTCCGTGCCTCAGTTTCTCATTTGTCAAGTGGGGACA
GTTCCTTCCTGTGAGGATTAAGTGAGAGTTTATATGAAGATGGATGGGTAGACTCAGAGCCTGTCATATAACAGGTACTCAGTAAGT
CCTTGTAGAGTTTGAATCTGTGGAGAGTTCCTAGAGGCAACACCTGTAGGGGAACCATGGAGATCCTTCTGCCTTGAAATGGCTCTA
ACTTGAACCAATTAGTGTGGAATGAGCTCCCGCCTTGAGATGGGGCTGCCTCATGTTCTTGGAGAAAGAAATGGAGGAAATAACCAC
ATGGAGGAATTGGGAGGTTCCCTGACCCCCCCTAGACCATGGATAGTCTACTTGGGAGACTCTACTGCATGCCCCGTGATAGGCAGG
ACCAGCACCCAAGGAGTCTTTGCATCTTGCAGTTTCCTTGAAGCCCTGTACAGGCTGGTGGCCCACGTTGGTGTGTGGCTTCCAGCC
TTCCTCCAGGCTCTCAGATGAACATGCTTGGACATGGGCAGGCAGTGGGGCTCGTGTCGTGCCCCATTGCTGGAATTCATGGCACCT
CAAAGTAAAAGGTAGGCAGGTTGGCAGCAGTCTGGTGCCAGAGAGCTAGTGAGAGAGAGAAAGAATTATACAAGTGCCATGTTTGGT
CTATTAATAGCCTGTGTTTGGTCTCCACCACTTCTATCAACATCGTCCATCACATCCAAACAATTGAAAAACCTGTGCCAAATGTTA
GGAAACTCACGGCTGTGCTGTTTCTGAAAAACAGCATTTCTACTTCTTATGCCGCACTGAGATGCCAATAGTTCCATGCCAACTGGT
TTTTATTTTTCCAGACCATTTCCCTTTATTTATGTTTAAGCTTTTGTATTGTTAAGCCAAAAGAAGATAGAGAACCTGGAGTAGGTA
ATTATTGTTTCTCCTGATTTGTCCTTGAATGAGGCATGGAGGAAATGTTCACTCCCCAAACCTGAATTGAGTAGCTGCAGAGGGGTG
GAGAGAGGACTAAGACAGGGGCCTGTCCCTTTCAGACAGTGTTCAAGCTGGTGGATGGGCAGAGTGAGGATGTGGCAGAACACCAGC
CCCACCCTTGGAAGGCTTAGGGCATTATCAGCCCTTTCTGTTACTGGTGTGTGGCCTTGGGTAGGCCAGAGTCCCTCTCTGGGCCTT
AATTTGCTTGCCTATAGAATCGGGGAGTCAGACTTGATGGCTTAGAAGGCCTCTTCCAGCTAAGTCGAGTGTTGGGTGGAGGACTCA
CTGTCTCACGTTTGTTGGCAAGGCCGTGGGTGTGGAAATGGAAGGAAGGTGATGCTGCCCTCAGAATGCTGGCAACTCCGGGAAGCC
TGGAAGCATGGCTGGGAAACACGGACGAGGCTGTTCTTTATCTGGGAAACTCCAGAACAGCCTCCAGAGATGTCTGTGGAGCCTGCA
GGCGGTTGTTTTTCTCACCATATGTGATTTCTGGGTGTCTTAAAAATTTTCATTTGGCCCAAACATTGCCGGGGTTGGGGGATGATT
TTGCCCAGTGTGGGGATGGAAATGGGTTTGCCTCTGAGCATCAGGTGGGTAGTAGTGGTTTTCTCCATTTTGGAATTCTCTGACGAA
GTTGGAAGGTTCAGCTGGGGCCCTAGTGTTGCAGCAGCTTCTGGTGCAGACAGAGGCAAGGGCTGGAGGGAGGAAAGGGGGAAGGCC
AATTGGAGGGGTGATAAGGCTGAGAGACCCAGGGTGGGGAGTTGTCTGTAGGCCTTGCTTTGTGTCTTTAAAACCCACATACCTCTC
TGTGGCTATCAGGTGATGAAAATCAACATTATTTCAAGTTATAGCCATCTGAAATGAATGTAGCCAGTTCCCTGTAATTAGCTGACC
ACAGCTCCTGCAGAGAATCTGGGAATCTGTTGTGCGTCTTGTCAGCGATGGCTGGGGCAGGATGGGGAAAGGGAAGCCCAGGCCTGG
GACAAGCCCAGCTAGGCTGGTTTGGTTCTAGTCAGCAACCCAGTCTAACAGGGAGCCCACAGCTTTCTGTAGAGCAAAGTGATTTCC
TGCTGTTTTATTCATTTGGGAGCCAGATAGGTTTTACTAAGAGAAAGCAAAACTACTTCAAAGCAAGCCATCCAGAAAGCACAATTA
TTTGTTTCAAATATTCTAGAACATTTAAGAAAATGGGGGTTCCTGCAATCTGGATGAAATATGGGGGCCAGATCCTTGGCAGGTGGA
GCAGTAAATATTTGCTAGGTGAACCCCTCATGGTGATGGCAAATTGACCAGCGTTCTGGGAGGCGCTAGGAGGGTGAATAAAGGTTA
AGTGTTTCACTGTAACTCTGTACACCCTGGATGCCAGGACAGATCTGGGGGCTGGGACTAGGAACCTTCAGTATGCTATACAGTTCT
CCTGAGATAAAGGTATGGTGTCAGGACCTTCAGAACCTGATGCATAGTGGCAGGTACGCATTCATCAAAGCCAGAGGAGGCTGTAGG
GACTTCTTGACACCACAAGAGTAATGTAATTGCTAACTGGCCAAGAGCACTCACATTCCACAAGCAGAGGAGGCTAAAAGCTCATCA
GGAGCCCATTTTTTTATTGGAGAAGGAAACAGTGACTAAGTGACAGTCTTGGAGGTGACTTCCAGGATAGGGATACAGGCTTTTGGA
GAACAGATGAATTCAGGGACCGGTTGTTATATTTCCGTTAGTATGAAAAGCAGGGTGGGCCGGGCTGCCTGCCCCTGTAAGCATAGG
CCGGGAGCCCCAACACCTGGGTTCTAAAGTTTTCTCCAAGCCAGGATGCTGTATGATGGGGGCAAGGCAGCCTCAGTGTCATCGTGG
AGAATGAGGGTTGGGGAACTAGCTGATCTCCTGGTGTTCTGTACGTTAATGAGTGTGTGTCCCAGGGAGAGGCTCACTTGTCAAAGT
CATTGACATAGACACCGGGATGCTAAAGTCAGAGCAGGTCTTCACAGAGGCATTTGTTTCACTGGGACCAAGCACTGCAGGCAGATC
TTTGAAAAAGAGCCATCCAAAATAGAGTCTCTCCAGGGCTACATTGAGAGGAAGTGAGTTTCCAACCCCTGGAGAAAATCAAGCAGA
GACTGGATGGCCATGTGCCAGAAATCTTAGGAGACTCATGGGGCACTGAACTAGATGGCTTCTAAGCTCCATTGCTTGTGTGTATGA
CTGTCTGTTTCACAGTGTCTCAGAGCTGTGAGGGCAACCCCAGCCTCCTGGCTTCTTTCTTTGTGCCCCAGAACAGCAATTCTCATT
TCTGGCCCGCACATTAGATTCACTGAGATCTTTTTTTAAAAGTTAGTGCAGAGACCTCACTTGCAGAGATTCCAATTTAATTGCTCT
GGGGTGGTGCCAAGCCCCTCAGTTCTACAAGGACCCGGGCTATTGGTGCTTGATGGGCTTCCATCCCACCTCCATCCTCCCTGTAGG
GGTGGGGAGTTACAAACCCTTCTGCTCCCATTCTGCCTTTGTATGGAGCCCAAAGGTTTAAGTCTGTATTTACCGTTCCATATTCAT
TTACCCAAGAAACACTTGTTAAGTGCTTGCTTTGTGTCTGCAAGAATCCTAGGTGGGCCAGCTTTTGGCTACCTGAGGAGCAGCAAG
GACCAAACTGCCTTTAAAAAGGCCTCATCCTGTATGTTTCCACCTCCCGCACAGTGCCTGGCACATGGCAAACATGTAATAAAAAGT
TTGATTGAATCGCATGGAATCTTTGCCAATATTGTAAGTGGGGCAGGAAAATAAATTATAAGCAAGCATGCAGTAGAGAATTCATGA
TATGCCAGTTCCCCTCTGCCCAGACCTTATAAAGGAAACCATCTCCTTAATTGGAATTATGTTTGTGGCCTGAAGGGGTGCCCCGTC
TGACTTGATATTTTGGGAGCGTGGTCTCCTAAATGACCTTTTTCTGCCTTCCTTGGGACATCACACAATGGGAGAGCCAGGGCCTGG
GGAGGATCATTTTCTCTTAATTGGTGTCCTGGTCATCATGCTGGCTTCACTAAAAATTAGTAGGCCACCCTCTGCTCAGACCTGAAT
GCCCTTGTTATTAACTGCATGATGTCCCTCAGACATCAGGTTCCGTCTCCTTCTGGGAGTGGCAACGTAGAGATCCAGGGAAATGCT
CCAGGGCAACTTGACTTTATGAGTTTTGTGTCGAGACCACTGGCTTCCTTCACGCCACAAGTATTATCTGCCTAGTTTCCCCGTGCC
GGGCCCAGTCTGCAGAGACGGTGAAGACCCCAAGCCCTGAGCTCAGTCTGTTCACCATCTATCACACCACTAAAGCCACACAACTGG
GGGCTTCTTCAGAGAGCAGGTGGGACCGGTAAGGGCAGGGCCTGCAAGGGTGATGTTCTGGCAATTGCCATCCTGTCTTCCTGAGGT
TCCCTCTGGATCAGTGGTGAGAGCACAGGTTTGAATCTAGCCCCCACCTCTTCCTCATCCAGGGACGGTGGGAATGTGACCCTCTCC
AGGCTTCAGATCCCTCATCTCTAGAATGGGGCCATGGGTTGTTCTGAGGACCTAACGAAATTAGCACAGTACCTGGCCTCTGGTAAG
TGAATAATAAATGTTAGCTGTTACTTTCATCACCTCTCTTTAGTTTTGATGAATTTGGAAATTCAGGATGGAAGTGTCTACCTGCTT
GACATATCTCAAAAATTTTGCTCCCTTCCTTCAAATATTCAACGTTCAACTAATATTTATTGACTTACTATGTGCCAGACCAGTGTT
CTAGGCATGGGGCTCTAGGAGTGAACAAATGCAATGTAAAAATTCCCTGCCCTCTGGGGTTGACACTCTGGTGGGGGTGTGAGAACA
ACATCAGTGGGAAAATGGGGTCAGATGGTGAAAGGTGCTCTGGTCACCCCAGCTGGGCAGTGCCAATGGGAGACACCAGGCATGGCC
AGCCTCGGGAAGGGTGTGGGGCCCAGGGCAGACCAGCGGCGTTCCTGGTGCTGAGGTTACCCTGTGTACTATTAAAGCAGGTAATGG
CTCCCAGCTTCGATGGTGGGTATATGGACCCCGTTCAGGGATGGGGGCTGAGGAGGGCACACGAGGACTGAAGACAGGGCTCTGAGG
AGCCACCTGCTCTCGGGTCTAGGTCAGACCCCGCATCCTGATGCGGGTCAGCTGGCTCATGATGCCTGGCTGGCTCCTGGGACTGAG
AGGAGAGTGTGGATGGTCTTGTCTCAGCTTTCCGCCCACTGGGGATCCACCTCAAGCATCCCCTGGCTGTGGGGTTGAGGGGTGGGC
CCAGAACAGAAGCTTCATGTAAGAAGGTGACTGGGCTGGAGAAAAGGTGGCAGTGAGGTTTAGCCCCATCTCCAGCCCCTGGGACCT
GTGGTAAAGATGTCCTCACCCACACAGTGGGCTCAGGCAGGCAGGAAAGGTTCCTGGAGCTTCGCCTCTAATATCCATTCTTGAGCT
CTGCCGTGGAGAGAGCTGGTCACTCGAGCGCAAAGGAACCACTTGTGGGTTTTCTCCCTCAGCCTGGGCTTCCTGCCCATTCCTGGC
TGTCCCAGCCTGATGTGTCCTCCAGGGCCCCCTGACCTGACCCTTGGCCCATCTCTTCTTGACCTTGCACTGTGGGCAGGACAGAGA
GGCCAGGGAGAGTGTCCAAGAGGGACAGGATCGGATGTCAAGGTGACGGGGCGATGATGGTGCAGAGGGCAGCCCTTGGGATGAGCT
CCTGGGAGCTCCTGGATGGGATTCAGGCTCAGCAGGTTGGGGTTGGAGCGGGTGGGGAATCCCTGGCATTTGGCAATTGCCCAGCAC
AGTGCCTGGGCATGGTAAGTGCTCAGTGAATATATGTTGTATGAATGCACAAGTGAATGAATGTTTGGCTATGCGGATTGCAAAGGT
GTCTTTCTGAAATGCCTCAGGGATATCTTTATAAATTATTGCAGCCATAAAATGCTGATATTGGGGGTGCAGTGAGCAGCTCCGGAC
TGAGGATCAGCTGCATTTACCTGCACTGACATCTGTATGGCAAGAGAAGGGGTGTTTGACTACAAGGGAATGCATGCTTCTCTTTGT
GTGAAAACAGAATGAGATTTTTCGTATGCATGATCTCATCTCATCCCCTAACAGCTGTGAGAAAAGTAAGGGTAGTTCTTGTTTGCA
GAGAAGAAACCAGAGGCCCAGAGAGGTTAAAGGACCGATCCAGTGTCACACAGCTAGTTAGTAGCAAAGACATGACTTGAACTCTGG
CCTTTTGGTTTCAAATCCTATGTTATTTAGAAAGAGACAAGGAACATCTTCGTTAGCCCCCCCATGACTAAAAATAGGAAGCTTGCC
TTGTTGTGTTTGAGGAGAAGATGGTGTGAGTGGGTTTTGTGGCTGACCGTTTCACACCCAACATTTCATCACAGAAATGCCTGGGAA
AAGAAAACTCCTAAGAAAAAAAAAAAAAGGAATGAGTGTTGGACATGTCCCTATGACCTGTTTGTCATGGCATGTGTGCTCGGCGCC
TTGGTCATCAGGCTTAACTGAACTTCGGAGAAGGTTCTGTCCAGGAGTCTCATTTTACTGGGGGCTGAGGCTTAGGAGAAGGAAGCA
ACTTGTCCAGGCCAGGCATTCACTATGGAGGAGGCTGAGACTCCTGCGCCCTCGGATGCACACCCTGGGCCTCTGTTCCAGAGCTGT
CTTGGCTAGCAAGTATCAGGCCCTTCTCCCTTCTCAGGCTGGTCAGAGGTCCAGCTGCCCTCTGGAGTCCCCAGAGTCTCCCTTCCC
ACTGTGACAGCATCGGCTGTCTCTCCCGGTCTCAGGTTGGACAGTCCTGGGAGAGAGAGGGTCTGAGTGACTTCATTCTGTCACTTC
ATTCATTAGTGACTGAAACAGGATTCTGTTAGCTGACTTCTGGTAGGAGCCAGGTTGGGAAGCCGCTCTGAGGATGCTGGGAGGGCC
AACAGCTGCAATTATTATTATTATTGTTGTTGTTATTATTATTATTATTATTGAGACAGAGTCTTGCTCTGTCTCCCAGGCTGGAGT
GCAGTGGTGTGATCTCGGTTCACTGCAACTTCTGCCTCCTGGGTTCAAGCCATTCTCCTGCCTCAGCCTCCTGAGAAGCTGAGACTA
CAGGCTTGCACCACCATGCCCAGCTAATTTTTGTATTTTTAGTAGAGATGGGGTTTCACCATGATGGCCAGGCCGGTCTTGAGCTCC
CGACCTCAAGTGATCCACCTGCCTTGGCCTCCCAAAGTGTTGGACTTACAGGCGTGAGCCACCACGCCTGGGCTTGCAATTAAATTA
GGGTGCTGCACCTGGGAGGAGGAGGCTGTGCTAAGATAGGAGCTGAGGATGATGGGTAAGAATGAAAGATCCCAAGAACTGGACAGT
GTGAGATTGACGAATGAGGAAGGCCAGGGCTTGGTGCTGATGTGGAGGGAAAGAAGCCTCAGGTCTCCATGGAGAACAGCGACCAGT
GATGCGGTCTTCCCTTTAGCGGAAGAGGGGCTGGTTTAATTTGCATAGCATTAATTAGCAAGAGTATCTAATTAGGACAACCATTAC
AGAACCACAGTCATAAGCCGATGTTGAATATCAATCAATTTTGGAGCCTCTCCATGAAATTGAAGGAAAAGTTGACCTTATTAGATA
GAAAAGATTTCTAAATCAATTATTTCCCAGATTGAATGCACAAAACATAGTGATGTCAGGCAGGCACATCAGACCTTAATTCAGAAA
TTATGAACTTCAGATACTATCACCTTTTCCCTTTGGGGGTTTCAGCCAGTGACAGAAATCGTGAATGGACCACATATGTGTATAAAC
ACACACACACACAACCATACACATATACTCTTTGTGGGTAACAGCTTGGTTGGGGAAGGAGCCTGCGCATCATTATCTCAGTTAAGC
TAATATTTCAGTTTGGTTCAGTGCCCCTCCAGGTCTCTGTCTGTAAGCATAGCTTTCTCTTTTTTGCGATGACCCAAACAATAAAAT
ACATTTAGAACTTCAAAGTCTCTGGGAAGATATACCATAATCATTACAGTTCTGGCCTGTTTTTGGAAGCTACAAGGCAGGCCAGGA
GAGCAATTTATGTGGCTGGCTCAGAAGCTGGGGAACACACTCCATCACCCTCTGCACAGCCTCAGGGGAGGAAAGGCTGGAACAGCT
CTAAGAAGCCTCGCTGCAGATATTGGAGGGCCAGCAAGGCGTGGCCGCCAGAGCAGGTGGGGTGCTGGGACACAGGCGGGGCAATGG
AACCTGGAGGCAGACAGATCTGGGTGTAAAACCTGGCTTTTGCAAATCCTGGCTTTGCCATGGTGAGAAGTTAGGTAACATGCCCTT
GGACAAGTTACCTAACTTCTGAGAGCTTAAGCTTTCTCCTCTGTAAGAGTGGGTTGACAATGCCATCCTTGCATGGTAGGCCCCTAT
AGATGTGCTTTATTACAGATTATTGTCATCAATGAGAAATCACAGATCTGTGAAGGGCTGGCTCGTGGCCTGGAGCAGAACAGGGAC
CACATGGATGGAGGCAGCTCTTAGGAATTCTATTGACATATTTTCAGTCTTTTTCTACACTAAAAAAGCTTAAAAATGAAATAATTG
GAAATATACTACATATCGTTTTGGGGTCTGAGTTTATCACTTAATATTCTATCATCAACCATTCTCCATGTCTTCAGATATTGTCTG
CAGAAGTGTTTTTTGGTTTTTTTTGTTTTTGTTTTTGTTTTTGTTTTTGTTTTTGTTTTTTTTTAAGATGGAGTCTCGCTCTTCACC
CAGGCTGGAGTGCAGTGGCACTATCTCGGCTCACTGCAAGCTCCGCCTCCCGGGTTCATGCCGTTCTCCTGCCTCAGTCTCCCGAGT
AGCTGGGACTACAGGCGCCTGCCACCACGTCCCGCTAATTTTTTGTGTTTTTAGTAGAGATGAGGTTTCACTGTCTCACCCAGGATG
GTCTCGATCTCCTGACCTCGTGATCTGCCCACCTCGGCCTCCCAAAGTGCTGGGATTACGGCCGTGTGCAGAAGTGTTTTTAGTGGC
TGTGTAGTGTTCCATCACTGCCTGATCTCAAATCTACTTAACCACTTGAAAATTTGTGGGCATTTAGGAATAAATGACTTTAAAACA
TGACTCTCAGCTGGGCGCGGTGGCTCACGCCTGTAATCCCAGCACTTTGGGAGGCCGAGGCGGGTGGATCACTTGAGGCCAGGAGAT
CGAGACCATCCTGGCTAACACGGTGAAACCCCATCTCTACTAAAAATACAAAAAAATTAGCCGGGTGTAGTGGCGGGCGCCTGTAGT
CCCAGCTGCTGGGGAGACTGAGGCAGGAGAATGGCATGAACCCGGCAGGCAGAGCTTGCAGTGAGCTGAGATCGTGCCACTGCACTC
CAGTCTGGGAGACAGAGCAAGACTCTGTCTCAAAAAAAAAAAAAAAAAAATGATTCTCATCAAATGGGATATAATTCCTAAGCATTC
CTGTGTGTTAAGGATTGTTACTCTGTGTTGAAAAGTTCTGAGTCACGGGGCACAAGGGTTGACCAGTTGACCGCTGGATTCCCTCTG
TTCTACAAGCCCCTTATTGGTCCTCAGATAGGAGTGAGTGGGGGCAATGCGTGTGGCAGGTACCCTATCCTGGGATGTATGGCAGCC
CGGTGTCACTGCCATCCCCCAAGGAACCATATCAATTTCAAGCTTCACTGATGTTCAGGCAGCAAAGAGAAGATGCTAATTCAACAT
TTTACCAAATAGAAAACATTCCTAGTTCAGCCAGACTTCTTTGACATGTTTTCCAGCTATTAAAAGCGGTTGGTTAATTTTTTTTAT
TGACATGCGATGGAGAATGCAAAGAAATCTGTAAGATATCGAGAGTCCCTGTTGATCCAGCACACAGCTTGGTCATCCCACGGGGCT
GGAGCTGGCCAGGGAGGTTGTACTGGCCAAAACCAAGAAGCAGGTGGGAGTTGCCTGTTCATGTAAAGGTTTTGAGCTTTAGGACAA
AAGGAAGAGGCCCAAAGGAGAGAGCATCTGGCCCCTTTCCCTTCACTGCTTCCTTTAAAATGATGGAAATAGACTTGGATTCTGATG
TCAATTCATTGCTGCCAAAAGAAATAAATAAGGGGCTGAAATGTGCAGCTGATCCCAAATCCTTCCCGGGGAGGTGTGATGCTCCCA
GATGCTTTCAGACCCCTTTGAAAAGAAAGTTATGGTCATTTCTGGATGGAAAACATTGTGAGCTGTCCCCAGTTGATCCCCAGTTGA
TTGATGTAGGGAGGTTTGTTTTACCCTAATACAGTCTGAGTGTTCCTGAATTCACCATTTGCTCTTTTCAGCCCCATCTGGGTCTTG
TGGTTGGGTAGCATTTCAGGCACCAGGAAGTGCTTGTGAAGATCAAAATATTTTTGAGGGCCAGCACTGTGTTCTGTCCTTCTCTGT
GCTTATGTTGGACCAGAACAGCACCAGCCCTCCTTAGGCCCCCATACATGTGTGCTGAGTAAATGATTGCAGGCTGCTCTTATGGAA
CTGTGGAACATGAAGAGGCTAAAAGAAGACTGGCTTATGTGTAGATCATCTTATGCATAGCAAATAATGGAGACCTTTTCCCATAGT
TGGACGACCCCATGCAATGTAGAAAGTCAGAGTTAGTGAATAAGCCAGATCAGAAGCTTCTGGTGTATATCACTGTATATATAGAAT
CTAGCACACTGTTGGATATCTAGCAAGTGCTCAGCAAATGAGAAGTGGTTGAAATTTTTGTGAAAGAGTGAATGAATTAGTTACTTT
ATTAGTTTTCTTTGTATCTCCAGTGGGCCTCAAGAACTGTACCAATTAAATGAATGGGGAGATTAATAGACATCAGGGAGAGCACTT
TCATTCCATCTCAGCACACAGAGTGGGGCCGAGAGCATGGACCTGGGCACTTGGAAGTCCTGGGTTCGAGTCCCAAGCCCTCCTCTC
ATTCTTGTGGTTGCCATATTGGACATACCGCCTCACCCCACAGATCTCAGCTGCTCCTCACATCTGCCTCTGAAAGCTGTCTCTGAC
CCATCCACAGTGGCCTCCCTCCTTTGAGCTCCCCTAGCTCTACTGTAGGTTTCACTCTGGCAATTGATCATACTCTACCTTACCTTG
TTGTCTTAAGATGTCATTTAGGTCTATTTAAATGGATGCTTCCCATGTTTCTAAGTTGTTTCCCATTGGAGAGTGTGCCTGAGGGCT
TGTGGGCAGTGCTCTGTACTGCGAGTCAGAGGCCTTGACTTTGAATTCTGGCACAAGCCACCCACCCTCCTCTGGGCCTCAGCCTGT
AATATCTATAAGGTAAAAGGCCGAGGGCTGTTGTGAGGATGGATGAGAACAAATACGTGAAACCACCTTGTAAACTGTAAGGTGCCT
TCCAGAAATCAGGCATCATTCTTCTAATGACCAGCTCCCTGAGGAAGGGAATGTTTCTTTTGTGGCGTGCACCACTCAGGACCAAGT
GCAGGGCCGCCATACACACAGTCACTGCTTGGAAATGCTTCCTGGATCTCATCTGCTGCCTTTGCTCAGGGCTGTGTTCCCGCCTTC
CTTTGGAACTAGGAGCTATCTCTACTTCCAGCTTCGAAATGCCCAGAGGTGCACCAAGTTCCAACCCAGACACCCTGATTTGTAGAG
GATGCAAAGTAAATGATAAGGGTAGTTAACACTTACTTGGCCAGGCACTGTTCTGGGCACTTTGCATACATTAACTCCTTTATTTTC
ACAACAGCCCTGTGAGGTAGTCCAGAATATTATCTCCATCTGACACAGGAGGCATGCTGGTGCAGAGAGGTGGAGTGGGCGTTCCAA
GGTCAGGTAGCCCTAAATGGCTGTCGCAGGGCTGGGACCCAGCAGTCTGGCCCCGGAGGTCATGCTCTTAACCACACTTCTCGGTTT
CTCTAAGCTGCCCTGGGACTGAAAGGCTGCAGTGGAGGCCACGGGGCTCACCTCCCCCACCTAATCCCTAGCAGGAGGGACCATCAG
GGTACTTCTCCTGCCAGGACCCCTCTCTGCTCCTGTGCTTCCTGGCTTCTCTCTCCAGTAGCTCCTCTGCCGTCCCTGCCTCCCTCC
CCTGCCCTTGGCCTAAGCCAGGAGATGAGAGCAGCTGCCGGGACATTCAAATTCATTCCTGAAAGAATTCTGCCTCCGAAATCCTCT
GGTTTGGGTTTAAGAGAAAACAGAGGCCCAAGCTGGCGTAATATTGATGCCCAAACCATGTAATAGCGGCACAACAAAGAATGACGT
TCATATGAGTTATGAGCATGGAAGCACAAATTCTAAATAACACATCAGCAAACTGAATCTAGCCGTGTATAACAAACATAATACATC
ATGCCCAAGTAGCGTTCATCCCAGGAATGCAAATACGACTTAGCCCGAGAAAACAGAGGCTCAAAGGGGGCAGCTGCTGAGACAGAG
CCGGCCCGGGCACAGCCCAAGGCTTTTGTGGGGCTCAGGCTGACTTTGTGCTACACCTGCTCCTAGGAGGGGCTGGTAACAGTCCCT
CCTGTGTCCCCACCCCTTCTCTTTTACACAGCCGCAGAATAAAGTGAGACGCTCCTTTCCCCGCACTCCCTAATTCTCTCTCTCCAT
TGTTCCTGCAGGACTCCATGTTTTCGTTGGCCCTGAAATGCCTTATCAGTCTGTCCACCATCATCCTTTTGGGCTTGATCATCGCCT
ACCACACACGTGAAGTCCAGGTAGGTGCCCCCACCTCACCCCTTCATGCAGCGGCTGATCCTTTGGCCTTCCTCTGAGCCGAGCCCA
TCCCCACCCCCAAAACCCACTCCCTTAGCAGCTACTTGGGTGATTTTACAAGACGGTGCCCAAGATCGTGGCTCTTCTCTTCTGGTT
TGGACCCAAATGGGAGAGGAAACTGCGAGGCCGCCCTGTGGCTTTCGAATAACTGTTAAGTTGTCTTCAAAGTGGGGATCCCAGAGG
CAAACATAGGGCTTCATGTTGAGCGAGGCTGCCGTATTCGGGACCCTCACTGAGCACCATCGGACCGGGAGAGCCCCTGATGCATTG
CTCTGCCTTTTGTGTCTCTTGTCCTTTCTGAACTTGGGCCACTCAGTTCATCCCTTCAGTGAGGCAGACCTGCCTTGTGTACCCCCA
CATATGCTGCACCAAACTCCACCAAGGAGTCTGCTTTTTGTGAATGGTGTGGTCTTCACCCTTGTGGGTCTAGGCTGGGGGTGTCAT
TTTCTAGACAGAGTAAGAGTCTGCTGTGGCCGAGAAGGCCTCTGTTTCTTGCTGCCCAGGAGTGACTTGGTACCCAGAGAAACATTG
GCTGGGCCTCCAGGGTGCACTTCCAGTTTGGCTGACATAGGGAGGATGGTTCAACCCGGGCCAGGAGGGTCAGACACGAGAGAACAG
ATCTGAAGCATCTTCTGGTGACAATGAGCTTCCCCCAGCCCCCGGCCCTTCAGGCTGACCTGCACTGCTGTGGACCTGAGAAGTTCT
AAACTATCCAGATGTTTTTCCTCATGTGCATGTGAATCTGGAAACCATCCCGGTCCTTTGGCTGGAGGAGCACAGATAGATTCACAT
CTGGGCAGCAGGCGAGAGATTGACGAAGACCAGAGCTCAGAGTCAGAAAAACTGGGGTCAAGTCTCGGCCATGACCTTTTTTCTTGG
TGTGTCTTTGGCCAGCCCTGTGACCTTCCTGGGTCTCGATCTTCTCATCTAGAACATGGGGCCTTTATTCCAGCTCTGCTGAGCTGT
TGTGAGGATGGGATGAGGAGCGCATGGGAGAGTGCTTTGTGAAGCGCTATTTAACAACACCGTTCAAATCCAGTCCCATTTGTTACT
CGGCATTTTCAGGGTGGAGCAGCAGTGCGGACGCAGGTGGGAGTGCCTCCTCCTCTTAGAGAGCCTTGGCTTTTTGTCTGAAGGTCA
GGGGCCACTGTGGCTGGCTGGGACATGGAAGCCTTGGCTCCAGGCCACCTTGGCAGGGCTGCATTCTGTCAATGACTAGAGGATACT
GACCTGGATGCTGGGACTCTTCAGCCTCTAAAGGGTTGCAGATGCTTGGACAGGGGCTGTGCCTGTGCCAGATGCCACCAGGCAGCT
TCCTTCATGGAGTCTGAATTGGACAGAGTGGTGGACTTTAACTCAGACCATTGTCCAGTCAGCCCCAGAAAGGAGCCAGGAGTACAG
GCAGGGAGGACTAGGGCCACGGAATCGAGACAGGACAGAAGTTGACAGTCTGGGCTCCGGGCCTGGTTCCACCCCACCCAAAGCCAG
CCGCGGGATCCAGGCAGTCCCTCAGTGTTCATGTTCTCATCTGTAGGGAGACTGTATTACTATGTGTCACCCTGACCTGTGTGGGGA
GGTGTCACACACAACAAAGAAAAAAGGCAGAACCATAAAACTCAGTTATGACCCAGGCTTGAGGAAGGGGAAGAAGAGGAGCTGTTC
TCAGCCTCAACCCCACACTATGGTGGTGCTAGGCCTGATTACTTCCTCTTTCTGCTCCAGGCATTGGGGTGGGTAGAGCCAGGTGTC
CCTCCCTCTGATGCTCCTGAGGTCCTTGTTCCCAGTCCAGGGCCTGCAGCCCTAGCTCTCAGCCTGTTCTGGTCCAGCTGCAGCTTG
ACTCAGTTCCGGCCTGGTGGCAGGCTCATGTTGCTGACAGCCCAGAACCTGGCTGGGTACCCATTGTCCCATGTCCAGCTCATGGCC
AATCTCTGTTTCAGCCCTAATCATGGGCAGTAGATGATGCTATTGGGGGTCATTCATTTTGGCCTGACACAATTTGGAGAAACAAAG
TGAAATCAGTTGTGTTGAATCCTGACTCTGGGGCTCAGTTTCCCCATCTGAAGTATGGGAACCACATCACCCACTTTGCTGAACTCT
TGCAGGGGTTGGAAACAGTGTAGAGTACAGAGTGGGCTTTCACAGAATGGAAACTTTGGTCCCTTTCCTGTGACCTTTGTCCCACTG
CTTCTAGCTGGGCCTTGCACTTACCAGCTGGGTCCACCAGGTGGTCACTATCACGACTGCCTGGTACTGGCAGGTGGCTGGGCTTCC
TCCTGCTCTGGCCCAGCCCTGCCTGTACCCTGAGCTCCCACCACCCAGCCCCTTATCCTCCCTGCAGCCTCCGTCACTTGAGGAAAG
TCACCAAAACCGGCTTCCTCTGTGCCTCTTACCTTTCCAGTAAAGTTCTTTCTGTGCCCAGCTAGACCTTCTCTTGCCAGAGGGAAA
TGGGAACAGTTTGGGCCCAGAGAGTCAGTGGGAGGGAGGTTCCTACGGAGCGCCCTTGTATATACCGCACTGGGCCATATTGTAGTT
TTCTGTTGGGTGTCTTTCTTCCCTACCTCAGGGACAGTTATCTTTGCATTAATCTTTGCATGTCCAGAGACTTATAGTGTCTGACAG
TATACTCTTCTCGGCAGAGTATAGCACGTTTGTAGCACGTTCTTTGAATGGAAAGCATGCTGAGTTCAAGGTAACTCTTTTCCAAAG
CTGGTCGTACCTTGACTTAGCTTTTTGGTCTCGGGCCTCAGTTTCCTCATCTATTACTTAGCCCCCCAATCTTGTAATTCTCTGATT
TAGATCTGGCTGCTCAGCCAGCTGCGGAACCTGCTTCCTGCGAGTGGAGGAAGGAGTTATCCCAAGTGTGACTGATGAAGTTATGCA
GAGGGGAGTGCTTAGCAAGCACCCTTCCACAGGCAAAGGCCAGCTGCTGCTCACCTGTTGCGAAGCAGGCAGGGCAGGGGGTGGCTG
CCGCATCTCCCCTTTGGGTGATGTCAGGACCTTTGCTAGGAATCTGCTCACAGCTTTCCTCATCCACAGTGGTTCCTGATCACCTAG
CTAAATTTTAGCAGGGACCAGATGATCATGTCCCCGTCTTCCCCAGCCTTGCCCCAGAAGCTCACACCCAGGACCCAGGCCCTGCCC
AGCACACATGTGAGGCTGGGGGAGTACCTTTTAGAACAGTATTCTTACTTGTTCATTTAGATGGCTTGAGTTTTTCAGGAAGAAGAA
TTCTAAAACACTGTTGGGTAACTCTCACCCAAGCTGGGTAATTTATAAGGAACATAAATGTATTTCTCACAGTTCTGGAGATCGGGA
AGTCCAACATTAGGGTGCCAGCAGCTTCAGTTGTCTGGTGAGGGCTGCTCTCTGCTTCCAAGACGGCGCTACGTTGCTGCATCCTCA
GGCAGGGAAGGCAGGAGGCTGAGGGACTGAACACTGCATGGGGCCTCTTTGATACAGGCCTCAATCCCATTCACAAGGGAGGAGCCC
TCATGGCCTCAACATCTCTGAAAGGCCCCATCTCTTGGCACTACCACATTGGCAACATCTGAATTTTGGAGGGGACACGTTTAAACT
GTAGTGTCCACCTAGTGCAGGAACCCCAGGGCCTCTGTGTGTGCTACTGTGACGGAGCTGCTGGCCCCCCTGGGAGAGGCTGCCCTA
CCAATGTCTTTAGAAAGCCCGCCCTTCTGCCGAGAAGAGGCAGCCTCCCTGGAACTTGCCCCCTGGCCTGGTTCTGCTTCTGGAGCA
GGGAAAAGAAGGGGTTGGTTTCCCGCATGGTAGCCTCCATTGTTGGAAGATGGCACCCCACCCCATCTCTCCTCAGTCTCCCTCTCT
GTTGGCGTTTTGGAGCACGGACCTTTCAAAGACTCAGGCACCTCTTTTCCTCAGGCCAGTGTGTCCGGGAGAGAAATGAAAACACAA
GAAGCAATGCATTTTGTTTATTTACCAAAACTCTGACTGAAAACCATGCTCTCCCTTGGAGAGTCCATTTACTTCTGTGGTTTAGCT
CTCACCAACAGCTCTCAGACCGTGCTAATCCCCTCCCCAGGCTGCAGGCTCACGTTTTACATGCCACCGGCAGCCCCTGGCTCCACA
CACAAAACACTGAACTCCTCTTGCTCCCCAGTATCCTCTCCTCCTTTGCCCTTGATGACCCACCAAGTGCAGTCATTGGTTCAGCAT
CTATTTAATAATGTTCTTCTAGGTGCAGAGCCCAGGACAGGAGCCCAGTTCTCTAGATGAGAAACTCAGAATTCACCATTGCTTCTC
CCAGGGGTCATCAGCCTTGAGTGAATCTATGGCTCTAATGAATGGCCCTGGTGCCATCCTGGAAGGCAGGCTAGCAGGCAGAATCAT
AAAACTCAGTTATGACCCAGGCTTGAGGAACGTTCTCTACTGAGAATGTAGACTTCAGAGCAGCCCAGCTTGGCGCAAATCATGCTG
TTTGTCACATTTAGTGTGTAGGTGGCCTTTAGCAAGTTACTTAATCCTTCTTAGTCTATTTCCTTTCTGTAAAATAGGACTAATAAC
CATACTGTGGAGGCCACAGCAACTCCATCTTGGATGCTGATCCTCCATATTGACTTCCGATTAACCCAGTTTCGGGAAAGCCTCTAA
GATTTCTCCTTTATCTACTGTTTACTGTAAGTCCTGCCCTTAGGCAAATTGCTTCTGAAGCACATATACCCCTTTCCTCTGGTATTT
AATCCCTGGGACTAGGGGGTCGTGGTGCAGGATCCACCATCTTGTCTTACTGCTGCCAGAGACTGTGGCTTTTGTTCATAAGTCCCT
ATTAAATGCTTCTTTCTGAGAAACTGGATATGTCAGCCCCTTTCTTCAGCCTTTCAGCTTCCTCAGCCTTTGGGGGTAGGTTTGAAT
ATGCCTACCCACCCGGAACCAGTACCTACCAGAAAGGGTTATTGTACAGATTAAATTAGATAATATATGCAGAACTCCTTAGCTTAA
GTAAGAATGTGTTTGTCTGGCTGACAGAGTGGAAAGGATACCCCAGACAGAGGTCTCAGAGTAAACAAAGGCTCTGAGATGGGGAAC
TGCAGAGCACACTGGGAATGTCTCAAGGACAGGGTCCTGGGTAGAGGGGCAAGTTCACTATGGAGGGTGGGAAGGACCCGTAGGGAC
TTGTTTGCCTTGCTGAAGGGTTTTGGAATGTCCTGTAGGCAGCAGCAGAAACTATGAGAAGGTTTCAAGCTGGGGACAGAGATGATC
AGATTTACATTTTAGACAGCACTCTTCATCTGCAGCATGCAAATATAGGAAAGGCCAATTTCAGAGGCAGGAGGACAGTGGGAAGCA
CGTCATCATGGGCCAGGTGAGGGGTGTTGATGCCCAGAGCTGGGGCAGGAGTGAGGAAGGGAGGATGGGAGGGATCCAGTACCCCTG
GGAGGAAGTGCCTGGCTCAGAGCCCCAGGCATGGAATAAGGGGTGGGGTGCAGACACATTCACATCCGGAGGCGTGGAGCAGAGCCT
GGCATCCTTAATAAAGTTGCAGGGATGGGAGGTGCCGTCCCTACATCCAGGCCAATGCCCGGCACGTAGCAGGTAGTCAGTGAATGT
TTATTGAATTAAACTGAAGAGACTACTGTGACCCAGCATGGGATACAGAGCAGTGGCAGAGTTCATATAATCCAGTCCCAGAATGGG
AGCTTCAGAGATCATTAATTTCAAATTGTGGTTCTTGGCCATATTCAAAGTAAAAGGCATCAATCAGATCAATGGGGTGGAGATCCA
CTCTTGCCTGGGGATATCAATCCCCTCAAGATCTAGCGTTGGTCTCAGATGTTTCCAGTCTTTTTCAAACTCAACAGAAACGTAACG
GTAGTAACAAACTATGGGCCTTCAACCTCATGAATTTGAAATACAAGGGACCAAAAAAAGGGGAGGCTTTTAATTTATTAGGTATAT
AAAAGTGGATATTTTCATTAAATACCAATAGAGTTCTGAGCAGACAGGAAAATTACTTGAAATTACAGGACATAAATTATAGGAAGC
CTCACTTCCTGTGGCATAATTGCTCTGTGGATGGGAGTGTCCTCCTGGGTGCGTGTAATGTGCTGTCACAGGGACATGGGTGTCTGT
AATGCAGGAGGTGTTGCTGGCCCAGGTGCTTTGGGGTCCCACCGCTAAATGAGTTAGTGGTGTCTTCCCCTGAAAGAACAGACAGGA
CAACTCACCTTGCGTCTTGCTCTGTGCCCTCACCCTCCCTTCTCCCTGTAAAAGAAAGCAGGCTCAAGTCACATCCTGAATGTAACC
TGTAAGGTGTCCTGGCACTGAGAGACAAGGTATAATAGCAGAGCACGTACTGACCTCTCTAAATTTTCCAACCCTAGTACAGTCTAT
AAAAATGGTGAGTCCATTCATTCGTTTACTTATTCAACAAACATTTATTGTAGGACTACTTTGTGCTAGGCATTGAGAATCCAGAAA
TAAAGCAGACCTGATTCCTGCCTTTGGAAATTGGTTTGTCCTAGACCTTTATCCACTGTGACCCAGGGTCATAAGTGCCATAATATG
AGGGTCTGTGAAACACCAAGTGGCCACGGAGGAGGGAGTGATGCATTTCTCCCAGAGAGGCTGGGCAGGTTTCACGCGGAGATGATG
CTGTGCCCTGCATTCGCCAGGAGCAGAGACCTTGTATGCTCAGGACAAAGCTGGCCCAGAGCGTTTCAGGAGAGGAAAAAAAAAGAG
AAGGAAGCTGTGCCTGTCCTCCGAAGGGAGGTGGAGGCTGCCCAAACCCCCCAGGTAGGGGCTAGAGTCCCCATCCTTCTTCAGACA
CCTGGTAGAGGTTTCCTAAGGGTGTCTCTGGCTGGTTGGTTGATGAATTAGAAATACCTTCATTGAACCTCCGAGATCTTGACACCC
CCAGACAGACTTCTCAGCCTGGCTAGGTTTGAGCATGTCAGCTCCTTAACTCAGGTGTGATGAGCAGATGGAAGGAGGGAGCTCGGG
CCACTCGTACTGAAGGGGAGGAGACCTTGACAAGGAAATTCCAGATGGGGGCCTCCTCTGCCAGGTTTACATCCCTCTCCTGTCCCA
CTTTCATGAGTGTGCCCCTGGATGATCACAGCACACAAGGGAAATTGAAAATGGGGAATGTGTTCCAGAGAACAGCGACTGGTGATG
GGGATTCACTGGAGCGCTGCTCTTCTTTGGGCCTCGGCCTCCTGCCTGGCCCAGCTGTGCACAGTGGGGCTAGAAAGAAAGGGAGAA
ATAGAGAGAGGAGCTACACCACCAACTGGGCCCGAGAAGTCAGAACACCATGGAATAAATCCTCGTCAGACTACACTTTGAAGACAG
AAAGCTCAGAGCTGATACATTAGCAAGCGCCTCAGAGACGATGGTGTGGCCACGTCTTGTCTAGCGCTTTGTATTTTTTAGAGGGTT
TTCACATCTATGAGGCCTTGGGCCTGGCCGCCTTCACCTTTTTGCCTTTTAGGTTTCAGCATAGATATTTCCTCCTCCAGGAACTCT
CTCTGGCCATCGTCCCCCTTCCCTGACCCCAGGGTGCCCCTTCTAGGAGTGGACCTAGCACCAGCTCTTATAGCAGCGGCTCTTACT
TGTCCCTGCGTGTCTTATGTTGGTTGTGAATACTATTTCTTGCCTGCAACTCCCTTCTCTGACTGTGGGCTCCATGGCAGTGGGGAC
TGTATTGGTTTCACTGTTGGACCCCCAAAGGCATGAGAGTGCCTAGCACATAGTAGGTGCTATTCAATGAGTGTAGGCTGAATAAAT
GAATGAATGAATGACAAAACTGGGGTAGGCATGGCAGATTTACTCCCTCCTCATTCTAAGGAGAAAGTAGAGCCTCTAACAGGCTGA
GAGCCTGACCTGAAGCCCACAGACCTGGCATCTGGCCCGTGCTTTCTGCATCCTAAGGGGCTGCGGGTCCCAGCAGGTTGCCCAGTG
AGCTGGCCATGCCCACCATGGCCCTCCCTGCCACTCTCTGGGAGGCCCACCTCGATCCCAGCACCTTTTCACTTGGATCGGCCGCTG
GCCATGGATTGCCATGGTTACTACACCAGCCCTGGCAGTCTCTGTGTGACATTTTGGTTTTATTTGCTAAACATTTAAACACTTGGG
CTTAACCCTTTCTGCCTCAGAGGGGGCTTTCCTGCCCCTGCTCCCTGTCCCTCAGGTGTGAGGGCCCCCGGCTGCTCTCCCTGGCCC
TGGCTGGCGTCTCCGATGGGGTGGGTGTGCTCGTGGTGGCAGCAAGGCTTGTTTCTCGGCTGCCTGTATTGACTTTCTGTTTTGTCT
CTGGGGAATGTCATCCTGGATCATGCTCTCACTGGGTGGTGCTGTGTCCATTAACAGGTGTGAAAAGCCCTGCTCCGGGCATGTTAA
GGCTTGACCTCCCCGTCAGCTGGGGTTCTGTTGACTTCCAGGCAAACGCCTCTTGCCAAGTCCTCCAGGAGCTTCCTTAGCTCCAGG
AGTTCAGGACGGGGTGGGGAGGGGTCATCTGCCCTTGAACCCTGGCAGGAGGACAGAGGGGAGACTGGTTTCGGGAAGAGTCCGTGT
TTGTGAATGGGAAGGCGGTGCGTCGCCTGGCCTCCCACGCCTCCTCCCCTGCGGGCTTAAGATGAAGCCTCCCAGGAAGAGGAGGCG
GCCCGGGAGGCTGAATCCACCCAGCTATGTGTTACCTTGGAAACCACACACTTGTCCTCGCCCTAGGCCATGTGAAGTGAGGGGGCC
GTGGACACGCTTCTCAGGAAGCTTCCAGTGTAACTCAGGATACAAAAAATCACAGTCCCAGCATGGGCAGCTGAGAGAATGAAAATG
TTCAAAAAAAAAAAAAAAAAAAAAAGCAGCCTAACAACTGCCCTGCCCAAGGGGCTCCCACCCCAGCAACCCTCTGCCCATTCTACA
TGGGACGAGAGGCCAGGGCCAGTTTGTCTGGGATGCTGATCACTTGTGAGCTTGGACAAGCTACTTGTTCTAGCTCAATCTCAGTTG
TGTTGTCTGTAAAGTGGGCAGATTAGGCCAGAGGATCTCTGAGAACTCTGATTGTAGCCAGAATTCCATGTCTGAGGGGCACTGCCT
GACAAGGAGAGGAAAATGTCGGGAGAGTGGCTGTGAGCGGTTTTTGGTAACAATGACTGACCTGGTTTGTGTTCAGGAGGCCCTTTG
TGGGACTGTCCGAACAACTTCTGTCCCTTCTCTTAATAGCCTTGCCTCTGCCCAGTCACAGAGCTGGCTGTCCCCTAAACACAGCCT
CAAACTTGACATAGAAAACAATAATAAAGACAGCTCTTTGGCTCCAACATCTTAAAGTTTAAAGGTGACTCCTCTTGAGTCTCTTAC
ACACAGGAGCTGGTCAGACAGCTTAAAGTTTTCAGGATAATAGAGTAAGTGGCCAAGTCCCCTGCCTTAACAATCCATTCCAAACTC
CTTCCATGAACGCGCACTCCCACTTCTCCAAAGTCATGGGGAACAAGTGCTCAGCTTAATAGAATATTCTGTTTCTCCATCATTCTC
AGCAAACTATCGCAAGGACAAAAAACCAAACACCGCATGTTCTCACTCACAGGTGGGATTGAACAATGAGAACACATGGACACAGGA
AGGGGAACATCACACACCGGGGCCTGTTGTGGGGTGGGGGGATGGGGGAGGGATAGCGTTAGGAGATATACCTAATGTAAATGACGA
GTTAATGGGTGCAGCACACCAACATGGCACATGTATACATATGTAACAAACCTGCACGTTGTGCACATGTACCCTAAAACTTAAAGT
ATAAAAAAAAAGAAAAAAAAAGAATATTCTGTTTCTCTGAGAGTGCCAAGGTGTGTCATCCCTGACTTTTTCAAAGTTAATCCGTTC
TATTTGGTTTTGTTGTTACCATTTTTCAATGATGATCATTTCCCCCGTGGCAGCGGTTCTCAATCAGGGGTCTGCCTCAGATCCCCC
GTGTCATATCTGGGTCCTGCATAGGCAGGTGAACCCCACTGCCTGGGAATGTGTGTTTTGACAGCACCGCAGGGGGGCCAGTGCACA
CTCTTATTAAGAAACATGGCACTCAATTAAGCTCAGGGAGGCCAGCGTCTTCCCAATCATCCCTGCAGCCACCCCTGCTGTGCCTAA
CGAAGAGCACTGTGCATAGTAGGACTCAGTGACTCCTGGGGTAAGGATCATTGACTCTCTGAGAATTTGTAAAGTTGGAAGGCACTG
TTGAATCTTTAGTGTTTGGAGGCTTCTATACAAGTGTTGTTGCATTGTGCTATCAAGAAAATTCTGATAATTCTAGTTTCCTGTTGA
GTACAAATCTAGTAAAATGACATTTTCCTGTTTTTAAGAGAAAAAATGGCAAAATCACCACTGCTCAAATTCTCCAAAACCCAACAA
TTGGACGAGGAAAAGAGATGAACTGCCCCGATTTACCTTTGAATTAAAGAATTCGATGCAATTCAATTGAGCAGTTTTGTTTCCTCC
AAAGGCATCTCCCATACAAACCTGTCCTGTTAGCATGATGGTGGGAGAGACACTGAGCCTGCGGTCCGTAGACCTGGGTCCAAGTTG
AGACTTGTTCATTTAAAACACTTTTTAAACTTGTGGTAAAATATACATAACCTATAATTTAGCATTTTAACCATTTTTAAGTATATG
GTTCAGTGACGTTAATTGTCTAATTATATTCTTATATATATATTTGTATCCTTATTATTGTATATTTGTCACCACCATCCATCTCCA
GAACTGACTTTTGTCATTTTCTGTGTGGCCTTGTGTGCCTCCACCAAGGCTTGTGGTGAGCTCTCTTCGCACATCATCTCCACAAGC
ATCTCCCACTTTGTCAGCCCCTAAACTTACCTTTACCCTGTCCCATCGCCCTAACGTCACGGGCCGGGGTTCTGATTCCAAGTTTCG
ATTGGGGAAGTGGTTGGTAATCCCACAGGTGGTAATAGTGTCGCCTCCATCTGTTGATCTCTTGTGTGCTGGAGACTGTGCCTTTAC
TTTATCATCTCAGTGATTCTTGGCGACAGTCCCGTAGTTAGGCTCTGTATTCATACCCCCTTTATAGATAAAGCAACTGAAGCAGAG
AAGGTAAGAGATGTGTCCACAGTCATGTGGCTGGTGAGGGCAGAGGGTTTTGAGGTCAACTGTTTGAGTTGACCATCTCCAAAGCTA
GCCAGGTTGCCCTGCCTGGGGGAGAGAGGCCCGTCCTCAAATTAACTCTTCTACCCCTGGTTCCTACAATGGCTAGAAGCAGCCTAG
AAGGTGGGCTGAGTCATGGGGACTTCCCTCCAACAAGGGTGAATATTAGGAAGCCTAGGGCTGAACAGTAACCCAGTGTTGGTCCAA
CCTTGCAGCAGCTCTGGAGGCCCTCATCCCAGCCACACCCCCACCTTGGCTGTGCCTTGTGGTGGCTCCTCTGCCTATTAAAATCCC
TTCCTTCTCCTGGTTTCCCTATTGTCCTCCCCTTGGGTCCAAGGCTTGCCTCAAGGATGAGGCCCAGCACCCTTGGGGAGGTAGAAG
GGCAGCCATCTCAGGGTAACCTGCCCAGCCATCAGAACTGGGACCCGCTTCAGTTTCTCTGCCTTGCCTTCTGATCCCAAGGAAGGC
TAGGGTCTTCCTTGAAGCCTCACTGAGCCCCATACTGGGTCTTGTTCTTTCTTGCCTCCCTGCCTTCTGAGGGGCTGACTTCTACTT
CTGCTCGCCTGGAGTCACAGTGTCCCTGATAGTCCCCAGGCCCTCAGGATCAGACCTGCCTCTAACCCCAAGCCATCATCTGCCTGG
GCTGAGTCTGGTCTTTCCCTTAGACACTGTGCCTTGGGCTGCCAGTGGCCTGGGTGAGTCAGGCTTCAGCAGCTCTTCCTGTTTCCC
CGCCCATATTCTCCCCTCCTTCTGCCCGTTCCTATGGTAAGGTGCCAGCAGCCTTCCACCCAAACTCCCCTAAGCTGGACCCAGGAC
TTCCCTAGATTAAATGCCCCCACTTCCCTGCCATAATCATGTCAATAATCCTGTTTCCACCCTGATCTTGCTACTCTGAACAGGATC
TGCTTGGTCCACAGCAGCCCAGGGATCTGGTGACACACTGACCCTTACACACCACTGGGATCAGGTCAGTACAGAAGGCACCGACCT
GCTTTCTCACTTTGTTACTGCAGCCAAAGACAGCCAGTGACTTTTAGCTTTCATGGTAGCCTGTTAGTTTATATTGAATTGAATATT
TACTAAAACCCCTCTGTCTTCTCATGAGCTGCTAAGCAGCTCCCCACCCTCTGCCCCCTCAATCTCCCATCCTTCCTAATACCGTTG
GCTTTTTAGGTGAGAACTGTACCTTTCATTTTCTTCTGTTACGTTTCCCTGCAGCTGTTTCAGCCCTTCATTCTAGTCTGTCCAGGC
CTTTTAGCATCTCACTCAGCAACATGTATTTGGGGGCCTCCCAGCTTCCTGCCCTCCCACATGTGATGAGCCTGAGTATAATAGCTT
CATCTAAACCATTGATACAAATGTCGAACCAGAAGTCAAATCTGGGAGGTAAACTTTCTTTGGTTGGACTAAAGACAGGAGAAGTAA
AGAAGAAACTTCAAGGATTTGCAAATCTTTTTCATCTTAAAAGTTTTGTGGCCTCCTGTGAGGTTGTAGTTTTATGTGCCCTCCCAC
CCCAGCCCCCAAATAAGCCTGCTCTGCATTCATAGAACACGTGTTTATTGAGCCATGCTCTGTGAGGGGCACGGTGCCTGGTATGGG
ACAGAGCTGTGAACTACAGACTGGACCCGGTCTCGTGTTACTCACAGTCTGTGAAATGAAAGCGTGGCTGCTGGTGCTGGGGCTTGG
CTGCTGGGGCTGAGTGTTGAGTCTGTGAAATGGACGGGTGAAGCCGTACACCTGGTCACTCGGTGAGATCACGTTGTGTCACAGGAG
AGTGCTGAGGCATGGCCACCGTGGCTGGACGTTCTCAGAGACACTGCTCAGCCACAGCAGCTGGGCCTGGCTATGACCACTGACCTG
TGACTACATCAGGCACAGCCACAAACCAGAGATCAGTAACCAGACACGGCGGAGCCATCCAGTGGGCGCAGAGCAGCCACCGCCTCT
GAACTCAGAGCCACGAGGCGGCTCTGCGGGAGCCGCCCGACACCTGCCTGCTGTGTGATCGCCAGCAGACCCTGCCCCTCACTGCCC
TCAGATCCCTCACTTGTGCATCTGGGGAGGAAGCTAAGCGCTCTCCAAGGTCCCTATCAGCTCTTTATGGTGCTTCTTGCTAGAATG
TGCTAGAAACCATATCGATTTGAGAGCAAAGAGAAATTGCCACCAGTGGGTGTGTATGTGTGGAATGGGAGGCGGGGGAGTGGGCAG
GGCGATATTTGTCTTCCTGCTGCTGTTAGAAATAGCAACTATTCTCTCCCCTGAGGACTGGACAAAAGGTGATTAATTCATTCAGCA
CAGAGCTATTGAAGGGCTGTTCTGAGCCAGGCACCGTGGGGACAGCAAGGTCCCTGCCCTAAACAGGCTTGTGATCTAATGGAGAGA
TAAGCACGCAGACACACACGCCTTGGCTGCTGGTGCTGAGGACAAGGGTGTGGAGGGCACGGCGCACAGTTCCACCTAGATGGGAAT
GAGGACGCTGCTGTGCAGGGCATCTGAGATGGTCATGGGGTCAGTTCTAGCAGGTGCAGGTGGGTCGAGGGTCCTCTAGGGGAAGGG
ACCAGCTCCAGCAGCTCTGAAGAATGCAGAAGCTCAGGGCTCTGCGGAATGGGAGGCATGCAGCTGATCGTAAAGGGAATGTTGAAA
GGTGAGAATAGAAAGAAGGGCTGGAGCCAGATCCTGGTGAGCAGCAGCTGGAGCGATGAGGCACCCAGGTCTAAAATGGTAGTAGCC
CCATTCTATCTATGAGAAGGGGTTGGGGGATGGCGATCTGATGGCGGGAGAGGGAAGCCAGGGAATTTGTCTTCAGGTTGCACCTGC
AAAGCATTTTATGTAAGATGGAGAGAATGTCAATAGTCTGTATCAAAGACTGAGAGGTAAGAATGGGGATGGGAGACGGGTGTAGAT
TATGGTACAGGTTGAACCCCCCTCAATCACTGGGACCTGCTCTGACCTTAGAGTACAGGGTTGGGCTAGAGGAATGGTGGATGAATC
TCCCTCCTTGGGAGATTGTTAGGATTAAAGATGAGAGCCACCTGGTACACTCTTGTTTAAGAGTCAGAGGAATGGAAAAACACCCCT
CCCACCCCAAAACGCTGAGGTTCTGATCTCACTGACTGAACACCACCCAACCAGGAGCCAGGGCTTGAGGGCAGCCCGAGTTTGTCT
TGCTCTGTCGGATCTTTTTGGATGTTTAGCAGGAGCGACACTGATCAGACTTCACAAGAACCTTAGGATTTTACAATTTTGCTCTTC
CCTTATTGTTCACAAATCAAGCCTATGCCTAAAAATACACTCTTCTCCAGCAGCCTCGGGGACTTAGCTTCTCCCCCAGGCAGGGAG
TTTACATCCTTCCTTTGCCTGCAGAATTCAACCTTGCAGGTTTGGTGGGGTGGCTGGAAACCGCCACCATCCCAGAGCAGGCTTCTA
TCCCCAGGGCCTGGCTGTCACCCGCAGCAGCAGTGACATCACCCAAAGCCGACTTTGTGCGGGGGGCTTCTGAGTGTTAAGTGGGCT
CAAAAGGTTGAAGTGCGGGGGCAGGTAGGAGGAGCTGCGTGGCTTGGGAAGGAGCAGCTGGCGCTGTAGAGGGAATGACTTGCCCCA
GCTGCTGTTGGGTTACGGGCTCGAGTTGTCCACAGCCACCAGGGGACATTTTTTCCTCTTCTCAGTTCCTCTTTGACCCTGCGCTCA
GCTTCACCAAAACCACCTTGTCTCACCTTCTCTGTGTGTCCTCGGCCTCTCCTTTCCTGCTTGCCTCAGTCTTTCCCATCTTTCAAA
TCTAAACATGTGGTGTCTGTTGCTGCGGGTTGACTGCATGTGTATGTGTGGTTTTCAAGCTGAATTGACAGGGCTGAGTTTTACAGC
CAATACTGCCTGGCTCTGGAAGAAGAGAAAGAGGAGGACAATGAGGGACAGGATCTCTTGTAGCAGAGAACATTCTGATCCAAATGT
CCTTTCAGGATAAATTTAGTTCCCGGGGCTGGGACCACATTCTAATTTGGTGGGAGTCCTTGGGCACCCTGGTGGGGAATGGCTGCT
TCCTTCCTCACCTCCCTGGTCGATTTCAGGGACCGCAGGAAACCCAAGCCTTATGAACATTATACAGAACTGCAATTACAACAATCC
TGAGCTGACATAAGATTTATTCATTGGGCACCAAATTTCCTTTAGGCCCCTTTCTAGTTTGTAGTTTTTGAAGCTGCTACATTTTTT
ATGGCTGGCACATCTCAAGGAGGAAATGCAGATTAATAGTCTGTACTGGCTGTGTTCTCATGCTCTCAGGATTGAATCAATAATGAC
TGCAGTATTAATTAGGCCTTTATGAAAAGAAAATGAAATATGGTATGACATGGAAGATTCTAGATTTAGAATCAAGAGTTCTAATCT
CAGCTCTGCTGCCGTCTAATGGGTGACCTCGGGCAAATCACTGAATTTCCCTGAGTCTGTTTCCTCCTCTGTATAAACAGGTCATAA
TGCTGGCCACATGCTGATGTTGTGAGCATCAGCTGAGAGAGCTGGGTTAAAGGGCCCACCAGGGCCTGGCACAAACAAGAGACTTCG
GTGACTTTAATTTTGGACTCTCCTTGCTAAGGAGATCACCACTGCAAATCCAGCCCTTTGCTGGTGCACAGTGGTGCAGCTCTTGCC
TGTTCTTGGCAGCAGGTGGGGTACCCAACACCTTGAGATGAACTCTCCAAAGTCCTTGCTGACAATGTCCACGGTCCCTTCTTAGCT
GGTGACCAGGAACACTCCCTAGGGGCCTCCCTTCTCAACCTTCCACTTCTCCTACCCATACCCCACAACGGCCCCATTTCCTGTTGT
TTCTCATGAATAGAGTCTGGAAGTAGAAACACTAGTTCGTGCAGTGTTCACGGACATCACGGGGCTCTAGGAAGCCTCACATCACAT
CACTCTGACACTGACTCTCCTGCTTCTCTTTTCCATATTTAAGGATCCCTGTCATTATGTTGTGCCCATCTAGATAATCCAGGATAA
TCTCCCTATTTTAAAGTCAACTGATTAGCGGCCTTAATTCTATCTGCATCCTTAATTCCTGCTTATAACATAGTCACAGGTTCTGGT
GATTTGGACACAGACATTTTGGGGGGCCATTATTCTGCCTACCACAATCCTCTATTTGTAGATGAGGAAACTGAGGCTAGGGTATTA
GATCACATACCCAAAGTCACACAGCCGGTAAGTGGCAAGGTTGGAATTTGAACTGGAGCTGCACAATCTGAAGTCTACTACTACCCT
AAAGGGCCCCCTAGAAACCCAGAGAAAGCTCATTCCCACAGAATGGCCCCAAACAGCCGACCACAGCGGGAGAAGTAGAGATCAAAA
GAGCCCACCCATTGGAATCCCCACACACCGTTTTAGTTCATAATGCAGAGAAGAGACTGGTGAATAATACTGCATAAACTCTGAACC
AGTGCTTCTCACATTTGGATTGAGAAAAAGTAGTTTGTAATTGCCCTGGGAGCTTTAAAATAATACTGGTGCCTGAGTCCCACCCCT
AGGGATTCTGATTTAATTGCTCAATTGCTCAATTGCTCAGGGGAGGCATTGAATAGAGGCGATTTTTCCAAGCTCCCCAGGTGATTT
GAAAGTGCATCAGAATGTGAGAATCATTGCCCTGAACTAACCCATGGCCACAGCCATTCCCCCTACCTGACAATTGTGTCCATAGTC
TGATTAGACCATGCCCAGAACACACCCCTCCCACCTTTTGGGCCTTTTCCCTGAACGCCCAGGTCCCCCTGTTTCCTCTTCACCCCC
TCACTCGAACCCTGACTGTGTGCCGAGCAGCATTAGATGTGTAGAGAGGACCCTCCCATGGGAAATGGGGCATATTCTCTCCTCTGG
CATCTCTCACGCCCCTCTTGCATGGCTGTCTGCAGAATAGAATTGTTTTAATGTCCTCTTTGTATATGCTATATTTCTATAGAGTCT
TTCCTTGAAAATGTAATTCACTATGTTTTCGGTGTCTGCATCAGTTCAGCTTGAAAGGCAGTGTGTGCAGAGGGTTGGAGGTCTGAC
TCTGGAGCCAGACTGCCTGGGTGCAAAGCCGAACCCTGCCAGTTCCTGACTGAGCTTGGGGTGAACGATTCTAGCTTTCTGTGCCTC
AGTTTCCTCATCTATAAAATAGGGATCAGAATAGCCGTTACCTAGGGTTGGTGTGAGAACTAATGCTTTCATATATGTAAAAGGGAT
TAGGACTATGTATTTGCTCATGTTACCAGTATTGCTACTACTCTTGTCTTCACCGAGATCTGTGGGATTCAGTTAATTTCAGCAGAT
ACCGATTCCCTACTCAGTGCCAGGATGGGAAAGAATAAGATGTGTTCTCTGCCTTCAAATAGTTCATGGTCTTGGGAGGGAGAGGGC
TGGTAAGGAGAAGGGAAGGAGTCAAATAGGTGAACAGCTAATTAATGATATAATACAGTGTTGACTTGGAACCACGTTGCTGTGGAA
TAATAAAGATGGGGACATCATGATGTGGGGATTCACCATGGTCGTGAAGGCATATCATGAATTCTAAGCACTAATAAAAAACTCTTA
GTGCGTTTTGTCCTGCCATCCATCCGCATTTGCGAAGCTGTGGGCTGTAACACCCTGTTAAGCCCCTTAGACTCATTTCATGAGTTG
TGAATTCGAGCCAATTGTGTTAGTGTTTGTGAGTGAACGCATTAATCTGAAAGGTGTCAGTGGCATCCCAGTTCCTGCTGCAAATGC
CCCTCACAGGGAGAGGAACGGGGTGGCTTTCTCCAAGAAAACAGCCAGTCTCTTCAAATCCTAGCCCTACCCTCCTTTCAAGGTGCA
TCCCAGAGAGCGGCTGGAGTCCACTGCAGAGGGTGGTAAAGGAGGACGTGTCATGCCAACAAGGGAGCAATGACAGCAATCAAAAGG
GACATCCAAGGAAAATGCAGAGTTGCACGGGGAGACGGGGTCTTAAAAGCCCACACAGGCAGGGATTGGAAAATGCTCCTCTGGGAA
TGCTCATGTGGTAAGGTGGCCATGGGTGAGTGGGAAAGGAGGGAGGGGTGAATCCTTGGTGTGTGAGGCTCTGTAGAGAGAATGGTT
GCTGCCTGGGCAGATGCCTCCTTCCTGTCCCCTCCAGCCAAATGGTCACACATCTCTCGCTCAGATTAACAGCTTGCTGGCCGGTGT
GGCGCGCAACTGTAGGAATCTCCGTCAGCTGTGGGAACAGCTGTACACTGCCCAGGGCCACATCACACCCTGGCATGTTTCCCATTC
CCTCTTGGTCAAACACACTTAAAAACAGGACTCAACGCTCCTTGGTTTCTAACTGTAAAATATGCCACGTGAAGGATGCAAACTCAT
GTCTGCCTGGGGACTACAGTGGGGCAGGCAAATGTTTGATGGGCTTGTGAGTTTTTGTGTTTTGAAGAAAAACAACTGTCTCAAGGC
AGACAGATGTTGATGAACGTGTTTATTTATAAAGGGCTTACTGAGCTAATAATGACACCAGATCATCCCCAAATGACAGGTGTCACT
GCTGACCTATAGCTCAGAGTGTCACTGCCTCTGTTGGAGCAACCTGCTCCATACTGCTGGCCTTTTTCATCTGCAGCCCAGTGTTGC
TTGTGGCAAAATGTTTACAGTAGCCTGGGGCTTGCGTGAAAGCCTTGCTAACCCATCCCTGTGCCCTGCTAGTGGTCAGAAATGTCT
GTTTTTGTTTCTAGGCTTCCAGAGTCACTAGGACTCTGGGCAGGGAAGGAGGGCCCTCAGGCTCATTGGTGTCTAGGGCCTGGGGGC
ATTTTTGAGCCACTTCACAAAGCCTGGCCACAGTCTGAAGTCCAGTACATACAGCTGCAGCCCAGGTGACTCCATGGTGAATTGCTC
CATCCCTAACATAAGGCACTTGGCACAGCCTCAGTGAGCTATGAGAGAGGCCAGAGTGGGAAGGTGTCATGCACGGCGTGAAAAGGG
CCCTCACAAAGACAGAGAGCCTCTCCCCTCCAACCAGGCAGAACAACTAAGTAGAAAGGCTGGTGCAGCTGGAGAATATGGCCACGA
AAGGAGCCTAACAGTGACAGCCAGAGGAAACCCAGGCCGAAGCCACTGGGCCTGTGTGGGTCTGCCTCTTGGATGGGGCTCCATGCA
CACCCATGTTAGTAGGTGGAGCTGTGTGGGAACATTCAGGTCCCTGGGGTTAAGTCTCATGCCTAGCCCTGGCTGGAGGGTTTTGAT
GAATGGATCTTTTTTTTTTCTTTAATAGTGTAGAAATTGTATTTTGCCTCATATTCTTGGGTTATCCATATTAGTCTGCAGCTGTTG
GTCTTCCTAAACCATGACTGAGTAGGTCATCCTCTGTTCACCACTCGTGATTTCTTACCATCTGGTGATTTCTGGATAATGCTGAGC
CCCTGAGCTTGGACATCAGGCTCTACAGCAGGGCTGGGCTCCATACTTCCCGGGCCCTGCCCCTGGCCAGCTGCCCTGAGGTCACTG
AACACCTGGGGTGAGAATAGATCTTACCCACCCCTGCTCCCTGTGTTTTTTCAAACTCCCCTCCTTCCTGGCTCTGGAGTGAGCTCC
TGGGTGCGGGGTAGTGGCTTGAGCTCCATGCAGGTTTGGTGCCTTGCTCATACTGAGAAGCTCAGTAAGCCCACATCAACCATTTGT
CTTTCTCTAGCCAAGAGTCAAATAGATCCACGTTTAAATCCCAGCTCTGCCTTTTATTAGCTGTTGGACTAGTTGTTTAAACCTCTC
TGCCTCACTTTCTTCATCTAATATCAGTACCTTCCTAAAGGGGTTGCTGTGCCCATTACATGAAATAATGCTCAGAAAGCAGTGGTA
CAGGGTAGGAATTCAGTATGGTAGCTGCTATTGTTTTTGTTACTATTATTATTTCATCTCACCCCTACTGCCAGTGCCCCGCATGCA
GTAAGTGCTAATAATTGTCCAATCAAGAAACAAGTGAACAAAGGCCATGTTCTTATTCCCTTGCTCTTGTGGGTTGGCCCCTTCCTC
TTCTGCTGCCTGCATGCCCACTCCCATCAGACTGCTGAGAGCGTTCCTGGCGCGCCCCGACTTGGTGCTGACATCAGCAAACAGAGC
CCCGGAGGCCGAGTCTTTCACTCCGGGGTTGTGCATCCTCCGGGGGTGTCAGCTCTTCAAGCTCTTCCAGGTCCTGCAGCTGTTGGC
TAGAGATGCCAGCTATGGAAACAGCTGTGCCTCCACCATCTTGCCTCTTCCTCCCCCTGGATTCCTGCTATCCCTTTGGCAGGCAAG
CGCACTTAAAAATAAGTGGCAAAAGTTGCACTTGTTTGAAGTGTAAGATATGTCACTCTCAGAAAGAGATTTTGTTCTTACTGAATA
AAGGATAAGAAATAGATTCCTAGTTAAAGAACTTTTTTGGCTAACTCTTCTTTCAGGGGGTGGGAGCGAGCATCCTTTGTCCTTTTG
GCGTAGGCTCTTGGGGAAGAATGGATGTTCTTGCTTCAGAGCCGCCAGCACAGGCTCTCTGGGCTCTGGCTTTCTCATTGATCTATT
TCCCTCTAGTGAATGGGTTTTACAGAAGCTGCAGAGAGATTTTCCCCTAATGATTGTGGATAGAAAAGAATAAAAAGTGACTCCGGG
GGTATCTTGGGACCTTAACTGTCATTGCACGCTGATGCTTCATTTCAGAATGCAAACGGCCTTCTTCTACTTTGCCGGGATAAATGG
TTCCCTTTAGCTGGGGACAGTGAGAAAAGAAGCAGGGCTTTCCAGGTAAAATTGAACGGCCTCCTGTGTTGGGGGTGAGGCTCTCCC
CCCCACCTCCTTTCCTCATCTCCCCCAGAATCCTAAAGAAAGGAGGACGGAGGCTGGAGTCCACTGGCCAGCTTGGTGTATAGAATC
TTTTTCTTTCTCTTTTTGAGCGGGGGATAAGGTCTGGTTGGAGGGAAGTGGCACCATCTTGGCTCACTGCAACCTCCACCTCCCAGG
CTCAAGCCATCTTCCGACTTCAGCTTCCCAAGCAGCTGGGACTACAGGCGCGCACAATCATGCCCGGCTAATCTTTGTTCTTTGTAT
TTTTTGTAGAGATGGGGTTTTGCTATGTTGCCCAGCCTGGTCTCAAACTCGTGAGCTCAAGCCATCTGCCTGACTTGGCCTCCCAAA
GTGCTGGGATTACAGGCGTGAGGTACCATACCTGGTGATGGATACAGTCCTAATCTGTTTCCTCCTAACAGGCATCTCCCGGTGAAG
CCTGGAGTCTAGGGAAGGAACTCCTGCTTAATGGGCCCCCAGGAAGTCACTTTCTCAGGCTGGCTGGCATCAGAACTGGTGCCCAGG
TAGGAGGTGCCAGTGTCAGTCTTCTCAGGGTGGGGTGGTGTCTGCTTTTCCTGTGGGTCTAGCATCCTCACCAGGTTATAACAAGGC
CAGCTCGGAGCTCTGTACTGGGATCTCTTAACAATCACCGGCCCTGAGCACCCATCTCCTGGGTGAGGGTTTGGGCCAGAAAGATCC
TCTGAGCCAATTCTGGACTCCTTATCTGTGTCCTGTGGTAGAGCAACAGCTCTGTGAGGACAGAGCAAAGCTAGGGGGCCACCGCCG
TGTACCAATATCAGCACAGGAGCTTGGCCCTGGTGGGAAAGCAATGGAAATCCATCTGCGATAGAATGGATGGGTGAAGGAAGAAAC
TTACACCTTCTCAAGCAGGGGCAGGTGGCCCTCATGATTCCAGACTGTGGCTGTGATCTGGGACAAGCTCTCCCCTTTATTTCCAAG
TGAGCTGCTTATATGTCCCATTCTGTAAGAACACAGCCAATGAATCATCACAACAGCACAATGACAATGATGACTTTGGGATCCAAA
ATAAGAAAATCAAACTCCCTTACATTCGTTCAGGAATCTACAGTTCAACGAGGCTGTTTCCCATGCATACGTCACACCCTTCGCCTG
CATGTGAGTGGATGAATGTGGCCTCCGTTTACAGGTGAGGCAGCTGAGGTGCTGCCAGGTCAAGGGACCTGCCCAGGACCATGCAGG
CAGCAAAAGGTGGGGTGGCTGCCTCCCAACGTGGGATGCAGAGCTGCAGTTGCCCCAGCACAAAGTCAGTTTCATGCTCAGCTACAG
AACTAATCTCAGTGACCTTTCCTGGGTCCCATGCTCTGCTGCCAGATCTCTTGTGGGATGAGGCCACCATGCTGACCAAGAAGCTCC
AGACCACGATCAGCTGGCTGTCCGCTGTAGGGGCACAGGTCTCCCCACCCCAGGGCACTTGCGGATTCAGGAAGGAGGCAGTGGAGG
CTCTCCTTGCTTTCAGTAGGAGCTAAGCCTTTTCAACTGCACTCTAGGGCATTTCCAGCAAATTCCTACATGACCCTATGCAGGGGA
GGGGACACAACCCTGGTGGCTTCACTTTATGGGTTGAAAGTCTCTGTGGGCTCCAAAAGCGGGTCAGCCTGCCCTCGTTTTCTGGAG
ACTCTGGCTACGCTCTTTGGACAGTTCACAGGGATAATCTCTAGAGGTGCCCTGGCTAGAACACGGAGATGCCTGGCACTGCTGTGC
ATTCATCCAATGAGTGACAGTGATCTTGAGCCCTTGACCTAATCCGACAGTGTGACCTAATCCCACAGTATGACCCAATCCATTTGG
CATTAATGCTGGCTGAGACTGGAGACCACTGACCTCTTTTCCTTTCAGTTTGATCATATAATAAGAACTGTGTTAGTATAAATCATT
ATTTAAAGTGTGCTTTAGGCCAGGCATGGTGGCACATGCCTGTAATCCCAGCACTTTGGGAGACTGAGGCAGGCAGATCACTTGAAG
TCAGGAGTTCGAGACCAGCCTGGCCAACATGGTGAAACCCCATCTCTACTAAAAATACAAAAATTAGCCAGGCATGGTTGTGCATAC
CTATAAACCCAGCTACTTGGGAGGCTGAGTCAGGAGAATGGCTTGAACCCAAGAGGCAGAGGTTGCAGTGAGCCAAGATTGTGCCAC
TGCACTCCAGCCTGGGTGACAGAGTGGACTCTGTCTCAAAAAAAAAAAAAAAATGCAATAAGTGTCAGTGTTGTGTAATTGACTGTT
AGTTCATAGCAATGGTCTCACCAGTCTGCCCACAGTATAGTGAGGTGGTAGGATGGAAGGCCTGGAACTAGAGACTAGGATTTGAAT
TTTGGCTCCACCACTTCTTAGTTGTGTAACTTCACACACTCTGTAAGTCTCGTGCCCCTAATCTGCAAAGTAGGTACAGTGTCAGTG
CCTACTCATTGAGTTTTTGTGGGGACAAAACCAGAGAATGCTTGTGATGTGTTTGTCACTGTGGTCCCCATTACCCTTAAGAAATGT
CAGCTCTTTTCTTTGCTCTTATTACTACATTAGTTTAAACAATGTCTCCCATAGAGGCTGCTCCTGAATTAGTAGTGTATTCTTGCC
ATAAATTACACTTGGGAATAGCCTGATTAATAGTCAGAGATAAGAGGCCTAGGGTGTAAACTCCACCAAATTGTTCAATGTGTGAGA
CTTTGAGTTAGAAGGCTTGGCGAATGTCCTGCCTCGCTATTTGCTGCCTTCATGGGTATAACTAATGTACCTTCTCCTCATCTGTGA
ATGGGGATAATGATGTTCCTGAGCTATGGAGAGGACCAGGCAAGCACTGTGCGTCAGAGCACCTGGCATAGGGCCTGGTACAGGTCA
ATGCCCACCTATTTTCTGTCCTTCCTTTCTTCCTGTTTGTTCAAACCAGGTCTGAGGCTAACAGATGTGTCTTGGTGAGTCCAAGAG
GGTGGCTGGGCCATAAGGGTTGGGGGAGTGGAGAAGTGGGGCTACCAGTTGCTGCTGGCAATTTGCAATATCTACCCTGTCCCAGGA
ATTGTACCCTGGCCTCTCGGGATGATTTAGTATGGCAGTCATGGAGCTGAAAGTTACAAATGGAGGCTCCAGTGATGGAGCTGGATT
TCCTGTTCTATTTAGTCCAGAGCCCATGTGGAGGATCCAAGACAATTAGAGCCAGAGAGTGCTGGAGAAGGCCTCAGTGATCATCAT
CAGACCATCCTTCTTATTGAACAGATGAGGAAACTGAGGCCTAGAGAGAGAAGACACCTAAAAGTCCACACAGCAAGTTACAGAGAC
TTGCTCAGGCAGAGCCACACCCCTGTGTCCACGCCTCTGGTCCTTCTAGGTCTCTGGGGTCACACTGATCAGAATTCCTCCAGCATC
TGCTATATGCCAAGCTTTGTGTTTCTCATTCAATTCATTTACCAAAAATTGAAAGAGTGCTTACACTGTTCCTGGCACTGGGGACAC
AATAATGAACAGGACAGATATGATCCCTGCCCTCATAGAGCTTCCAGGCTAGTGGGAAGGCACAGGGACAGGCAATAACAAGGAATC
AATCAAATAAGTCCGTGTAAATTCAGATGCAGACAAAGAACGGAATGTGGCAGTGATGGGGGGTCATGGAGGAAGAAATGAATTAGG
GTTGTAGCTTCAAGGGCGACCTCTGAAAGTTACTTTGGTATTGTGAGATTTATTTCTATTAATACTTCCTTTCGCTATGTACTACAG
TATTATCTCCTCAGGAAAGTGAGGCTCAGAGAGGTTAATTAACTTGCCCAAAATCACACAGCCAGTCAGTGGAGGAGTCAGAATTCG
AACCCTACTCTGCCTGACTCCGAAGTCCAGGTTTTTTCCACCATCCTTGATTATCTACAGAAATTAGGACAACCTACTACAGAATGT
CAGGTAATATGGTAACCATTAGTATGAGCATTAAGGGACCCCAAGGCCAGACCTGTGAGCCCAGCCTCACTGCTGTCTCTGTCTCCT
GTTCTAGGGGGAGGGACGAGGTGGATATTCTAGATGGTCTTACCCTCTCCTCTCCTTCGGAGACTGGCTTCATTGGGAGTGAGATGA
CACTGTCGCAGGGCTAGCTTTTTGTAGGCCACTACTTGTCGCAGGCTGCTGATGAAGCCAAGCAGATCACCCCCATGTCCACAAGCA
CACCGGGATTAGATACATTTAAGTCCTAGATCCCAAACGCCTTTTTCATTTCTGGTTTTGCATTTGTTTCCATTCTACTATCTTCAT
TTGGAAAACCCTTTCATTCCTCAAGAAAACAAGCCACACAATCAAAAAAAGATGTGTCTTATCCTCTGGGCGCTGCCTCATTGCCTG
CCCCATTATTGACCAATGCCACCGGAGTGCCTTGCACAGCTTGGCCAGCGTTCGCTGTGCACATCGACCAGTGAGCTGGCATGCTGA
CAGCGGCAGGGGCCCCCAGCTTCAGACACTTCTGAACCCAGCTGCTTTACGGACACAATCGGTTGAATCGGTTGAATCATGACTTGT
CTCCCAAACCACAAATTTGTAACTAGTTCACCGTGAGCCTATGCAGCCAGATGAATTGTAACTTGGGCAGTCCCTGCACCTAAATTT
TTTTTTTACCCAATAAAAAATGATATCTAGACATCTCTGTGTGCATGAAAGCATGTCTGTACGTGCGTGTGTATGTTTGGCACCAAT
GGGAAATACAGAGACGTGGAATGCCAGCTCTGCCCTCAACCTCTTCCCAGACGGGTGGGAGATGATATTCTGTGGACTGTAATTCAA
AGAAGACAGACTAATAACAAAGAGCTATGGAGACCAGGGGAGGGGCAAGAATTCAGATCTGGAGTGGAGACCAGGAAAATGGTCCTG
GGAGGCCAGACTCTTGGAGAAATGTGGACCTGGCAATAAGAATTTATGAGCTCTTTCTCAGTCACTGCTGAGTGTATATCTTCCAAG
GTATTTTCCAGTGGGCATAAATTACCTTTCGATTGCAGTATTCGCCACCTCCACTCATCCCCCAACACCAGGTCTTATGTGGACTGT
TAGGGAAGCCTCTAACTTGTACCCTGCCTCCTCACCCTTTCTGCACCACCTCACCTCATCGTCACAGCTGTGAGGAATCCAACAGAC
ACACACACTGGACCTTGTCCTTGTCTGAAAACTTTTGGCTGCTCCTCCTTGTCCTTAGAATACAGTCTAGACTCCCCAGGGGGGCAG
ATAGAGCCCTCCAGAATTGGCCCTGCCCTGCTCTGCGGCCACATCTGTGCCATTTCCTCCTGGAACACTGTGCCAGTCAACCTGGAG
TCCCCTGTGCCCATTACCCTGGAGTCCCCTGTGCCCCACAGGTCCACGTCTCTGGGCCTCTGCATGTACTGCTCCTCCTTCCAAGTG
CTGTTCTCATCTCTTAAGCCTGAGCTGTCCTCCCTGACCCACCCCCACCAACTTTCAGTGCCCCCACGGAGCCTCATGCATACAGAC
GTCTCTTGCTGCACCTCTTATATGCAATATATTCCTCTGTTTACATCACTGCTCATTTACTGAGTTATATGAGGGTGGTCCAGGCCC
GTGCCTCTTTGAGTCCCCATGCCTTATACAGCATCTGACAGAAGGGTGGCTCAGGGAATGTGTGGCACAGCAGTGAATGATGTGCAG
CTTCCACCGTACTTCCAGACATGAATTGATGGTCCTGGAACCTTCAGAGAAGCCTTACCACGTTGTGATCAGTAGGTGGGGGACTCG
ACACTACCACTGCAGAGGTGGCTTTGTGGGGTGGCCCCTGTGTGCGCTCTTATGTTTGCAAAGTGCGTTCTTTCACGCAGTCAGCCT
ACCAATGGGTGACCAGATTCTTTCTTCAGAAGCCAGTCAGAAAAGGGACTTGGCTTCCCAGATTACGCAAAGGCAATCTAATCCCAG
ACCTTCAGTTTTTGGTTAGCCAGCGGCTGCACAAGTGTCACAGACCACAGGGGCTGGGCGACGGGCATGGTGGTCCCTCTGCTTTCC
TGGGCACGTTAGGATGTCATTCTCTCACAGGACTCGTTTCAGAGAATTTTCCGTGTAAATGTGAAAGACCTAAATTTATGCCTGTCT
CAGTAACTAAGTATAGGCTACTTATGGAACCACCCAGCTATGGGAAATCCAGCCATGACAACTAACTGTGAAGAAATAGCCCCCCTT
ATTACACAGAGCAGACATCCTCAGGGATTGGATGAAAAAGAGCTCTCTAGGAAGTCAGTGAAAACGAGCTCCCTGCCTCCTCAGAGT
ATGCCTGGTCAATGTGTGACAGGCTCGCTACTGGGTTCCCATGCTGCATCCATAACAGACATAACCAATCGATCCCAGCATTCTTTC
TAGATGGGCTCAAAGTCCTTCCTCCCTCCAGTGCCACCTCCATGCACAATGCCTAGCCTCAGGCATACTGCTGCTCCTGCACCTCTG
TGAATGAGCCCTCCACAGAGGTCCACCTTTGGTCCATGGTTGATCCATGGACCAAAATGCCTGTTCCTTGTGGGCCTCAAGACAGCT
GAATAATCTACTAATTCAAAGGGAGACCAGAAAGGCAATAGTCAAAGATAATAAAGAGCATAACAATAGTCTACCCTCTATAATGCA
AGTTTTTTCTCCTCTCAGAGGGTCCCTGATCCCATCAGAGGACTCAGTTCTTCACAAGCAAGACTGATCTTGCAGCAGAGGGCTGAT
GGAGTCATTTACATGGAGAGCCAAAGAGAGTCTCTCCCACCAGGACCTTGAGCTCCTTTGAGGTGTGCCTCATTTTTTCATTCATCA
AGTGTTTATTAAGCATCTATGTTTAAGGGCAAGCTCTGGGGAAAACATGGACATAGAAACAGACATGGTCTCTGTCCTCAAGCAGCT
TATAGCCTCAGAACAGGCCAGACATTAGTCAGATAATTGCACAAATGACTGTAAAATTACAACTGTGGTAAGTACTCCAAGGAGCCT
AGATTGATCCAGTCAGAGGATGAATAGGATTAACTTGACATAGAGTGAAGGGAAGAACATTCTAGGCAGAGGGAACAGCATTCGGAA
AGATTGTGTGTCAAGAAGGAGATGAGCACAACAGCCTTGGTGGTCCTGGGAAGAGGATTTGTCTACATTCTACGCACAACAGGAAAC
CACCAGGCAGAACCTCTAGCCCTGGGACAGTGCTGTGCACATTCGAGGGCTGGATGCATCAGTGAGGAACAAAGGCATGCATGGGGG
AAAGGATCCAGTGTGATGGAATGACGGATGCTCTCCCACCCAGATTATGTGCAAACCCTGGAATGAATCCGCCTCCACACTGACTTT
GGAGATCTGGGGCAGGTCGGGTTTCCAACTGCAGGTCTTTAGTATTCCTTGACTAAGTCAGCTTAGTCAACTACGTTAACGAGGTCA
AGTCAAGCTAAGTTAACTACTCCAGCCATTCTTAGAGGTATTTCACTGCAGGCTTTTTGTGTTCTATTTCTGCCTTTCCCCCCACAA
CAGTTGGGTAACAGTCCCCAGCATAGTGAATAAGTTCTGTAAGATTTAATATTGATTTGTTGGAGTTTTTAAAAATTGAAAGTATAA
TTAATGATTTATTGCTACTTTGCAGTTTGCATTAATCACTGTCATTAATGGTAATCCTTTTAGGTGGTTTATAACTTTTAAATGGAG
TCTGTTAATTCCCAAGCCTGGTGGCTTTGTCTCTAGGCTGCATTGGGGTCTGCCACCCTCCCATGCCCCCGACATCCCTCCCCATCA
CCCCATTTGCTTTAAGATACAGGGATGAAACTCCAGGTGTAAACTTTTCAGGGTCAGCCTCTTACCAAGGAACAGACATTTGGGTAC
CTTCCTGGCCAAGAGGAGGGGTGGTGCACAAGCTGGCTCTGGGCCTTTGCTGTGGGCAGAGAGAACCAGGCCACTGCTAGCTCTTCA
ACCCACTACTTCATTTACAACTCTCTGACTTTGTATTCCCTGGTTTGCATGTCTGACACCTCACTTGGCACATGATTTATTACAGCT
TTTCAATAGCCCGTTCTTTCTGATTGACCTGTCCATCTCCATTTGGAGTGGAAAGATTGGCTTCTAGGTGTTTGTGGTGAGAATATT
CCTGAGTGCGATGGTTTGGGGTTAGGAGCTTCCTGCTCTTGGTACAGAACCGAGGACCACAACTAAAGGAACTCCCCTTTCTCTGGC
TGGGAAATGAAAAGGGTGGGGAAATACACACTAGAAGTTGCCTGAGACCTGGCTGAAGGAGAGAAACTTTCTGTTTTGCCAACACCT
TCAGGGATAGAAAAAAAATCAAACTTCTGCCTGCATCACAGGCAAGGGAAGGCAGCTCAATGGAAACAGCATGAAAAGAACTTCGGA
GCCCATATAGGTCAATCCCCTGCTTCTGGGGTGGGCATTCTCTCCCACATGAATAGGTCCAGATTTCATTTTGAAAACATTTCCCTG
GGAGGAATCTTCCCTCAAATTCTCTCTCTTTCCGTCTCTACCCTTCTTCAAGGTTTGACAATCTCTCTAACCAAAATCCCTCTTGCT
GTAGTTAGAGCCTCTTAGGGACCAGTTAAACTGGAATAAGGGATGGCCATAACAAAACATGCAAATTCCTTTTCCTTTTAAAGAGAA
ATATGTCTCCTGGATATTACCTGAATAAACCCATCAGCTTGCTGTTACTTTTCTGGGATTATACTTGATGAGGATGGTTGCAGAAAG
TCCTGTAAACATCTTCTATAGTCTGTATGGGGAGGACAAGTCCCACCTGTTTCCAGTGGAACATTCTGATTCCTGTTGTTACTCTAT
ATTTACAGAGTGGGCTCCACTAAGAGCCCACCTCCTTCTGTCACTGGTGGAAAGGCTATTTAATATGATCCCAATTAGAGGGAGGAG
GATCTGGGATGGTTGTTGGAACATGGCAGACGGGGGAGACCCGGAGGGAATAAACCACCCCAGGATGCTGTGTGACCACCTGACCCT
CTGCAAAGCAGCATCGTGGCACTCTGTTCCCTTTCAAGGTTCCCACAGTGAGTGCCACCCATTTTTACCACAATGGCAACAGGATTT
ATGCTCCCACCCACTCTCAGTTCTGAACTTTCTATGGCTTTGCCCCTCATTGGTCATAAAACACGTGTCACCTGCCTCTCTGACTTG
GCTTTTCTTGCAATTACATGGTTTCCTTTGCGATCTTCCTGGCCAGTGGAAGCAGAAGGATGCTTTTACAGACAGGGTATTATAGAT
GGGTTGGCAGATGAGGTGATTTCTGAGGGCCTGGCACAATGCCCAAAGGGACGTGAGTCAGCTCTCAGAGGAGGTTTGGTTTTTCCT
TCCAAATTCATACAAACAGCAGTGAAGTTTAAAAGATCCTCAAGTGGATTACAACCCCCATTCATTTGGCAGCAGAGGAGCACAACT
GTCAAAAAGTCCAGGTACTTTAAGCTGTGGAAGAGGCACAGAGTCACTCTCAAGTTGGACAGGGCTTCAAGCTATTTCTAAAATTAA
CATCGGGGCCTTTCGTTCCCTTTCGAGGTTCCTTTGAGGAATGGGTGAAAGCCACCTCTTCTTGACCACAGCAGTGACTGGATTTAT
TTTTGTCCTGTCTTCCTCCTCCACATTCTCAGTTCTCGAGGCTTTTTATTGTTTTTGGTTTTAGCCAGGCTTGCGCTGGTTGCTTAC
TCTCCGGTGTGCAGGGCTTCTAGAAGTGTTAGCCACAGCCAGCGGGAGGCAGCCGATCACCCATCCCACCTGCTGAGCTGTCGCGGG
CAGACATCTGTTGAGCTCCTCCCCAGCACCAGCCGCAAGAATGTACCTGATGTGGTCTTTGCTCTGCAAGAGCTCTCAGTTTAGAGC
AGTCTTTTTGAGAAAAAAAAAAAAAAAGTGGTATGATACACGTAATATAAAATACCATCCTAACTTGTTTTTTTTTTTTTTTTTTGA
GATGGAGTCTCACTCTGTCACCCAGGCTGGAGTGCGGTGGTGCGATCTTGACTCGCTGCAACCTCTGCCTCCCAGGTTCAAGTGATT
CTCCTGCCTCAGCCTCCCTAGTAGCTGGGATTACAGACATGCACCACCATGCCCAGCTAATTTTTGTATTTTTAGTAGAGATGGGGT
TTTACCATGTTGGCCAGGCTGGTCTCGAACTCCTGACCTCAGGTGATCCACTCACCTCGGCCTCCCAAAGTGCTGGGATTACAGGCG
TGAGCCACCACACTTGGCCTCATCCTAACCATTTTTAAGTATATAGTTCAGTGATGTTATGTTTACTCATATTGATGTGCAAACAAT
CTCCAGAACTCTTGTCTCCTGCAAACCTGAAATTCTGTACAGTATATACATGGGGGATGTGTTCCAAGATCCCTGGTGGATGTCTAA
AACTGCAGATAGTACCGAATCCTACAGAGAGAGATAGTCCCAGATTTAACGATGATTTGACTTAAGATTTGTCGACTTTATGATGGT
GTGAAAGCAACACACATTCAGCAGGAACTGTACTTTGAATTTTGAATTTTGATCTTTTCCTGGGCTAGTGGTATGCGGCATGATATT
CTCTTGTGATGTTGGGCAGCGGCAGAGAGCCCCGGTCAGCCCTGTGATCACGAGGGTCAGCAACTGATACAGTACTCTACCGTTTAC
AGTGTTCAGTAAATCACATGAGATATTCAATGCTTTATTATAAAATAGGCTTTGTGATAGATGATTTTGCTCAAATGTAGGCTAATG
TAAGTGTTCTGAGCACACAAGGCAGGCTAGGTTAAGCTGTGACATTCGGTAGGTTAGGTGTATTAAATGCATTTTCTACTTACAATA
TTTTCAATTTACAACGGGCTCATCAGGACGTGACCCATCATAAGTTAAGGAGTATCTGTAATGCTGCAATGAACATGGGTGTGCAAA
CATCCCTTTGATACTCTGCTTTTAAAAGCAAGTGTTCTTGGCTGGGCATGGTGGCTCACGCCTGTAATCCCAGCACTTTGGGAGGCC
GAGGCGGGCAGATCACCTGAGATCAGGAGTTCGAGACCAGCCTGGCCAACATATATGTTGCCATATATGTAGCGAATCCTCATTTCT
CTACTAAAAAATGCAAAAAATTAGCTGGGCATTGTAGTGCATGCCTGTAGTCCCAGCCACTTGGGAAGCTGAGGCAGGAGAATTGCT
TGAACCCAGGAGGCAGAGGTTGCAGTGAGCCAAGATCGCACCACTGCACTCCTCCAGCCTGGGCGACAGAGCGAGACTCTGTCTCTC
AAAACAAACAAACAAACAAACAAACAAACAAAAAAAGCAAGCATTCCTAACCTGGGGCCCGTGAACTCCCAGAAACTAGCTGCAATA
CATGGTGTGTTTCTGTAAATGCTTCTTGTTCTAGGGAAAGCAAATTCATAGCTCGCATCAGGTTTTCAGTGGGCTTAGGATGTCAGG
AGGGCTCAGTCTAGAGAGTAACAAAAGCAGCACCTCCTTCGGAATCCCCCAAGGCCCTGCCTGTCCTGAGTGCCGCCTCTCCGATGG
CAGGGCAGGTGTGAAGGAGCAAGTCATTCACTTCTTGGTTCCTGTCCCAGCTGTGCCATTTACTACCTGGGTGACACTTGGCAGTTC
AGCTGACGTCTTGGACCCCTAGTCTTCAAATCTGTAAAATAGAAATAATGACAACGTCTTCCTAGGATTGTTAGGAAGATTAAACTA
GATTTAGCACAATGGTCAAAAGCATAATGTGGTAGGCTGAATCATAATGCCCCCCCCTGCCCCACCGACCACCACCCTGCCAAAATA
TCCAGCTCCTAATCCCTGGATATCCTGGCAAAAGGGACTTTGCAGGATGTGGTTAAATGAAGGATCTTGAGATGGAGGAGAGTATCC
TGGGTTATCCAGGTGCGATCTAAACGTAATCACAAGTGTCTTTGTGAAAAGGGAGGCAGAGGAAGCTACCGAAGAGGAAGCAGGACG
AGTGATGGTGAAGCAGGAGGCTGGAGTGGTGGGAGAAGAGGCTACGAGGCAGGGAAGGCAGGCCCTCTCTGGAAGCTGGAAAAGCAA
GGAAACGGATTCCCCCGCCTGATCTTCGGGAAGGAGCCAGCCCTGCGGACAACCTCCACGTTAGTCTAGTGAAACTGATTCCACACA
TCTGGCCTCCGGAGCTGTAATATAATAAATCTGTGTTGTTTTAAGTAATGTGTTACAGCAGCAATAAGAAATGAGTGTACGTGGACT
CTGGGGCCAGACTGCCTGGGTCTGAGTAAACCACTTACCAGCTGTGTGACCTTGGGCATGTCACTTAATCTCTTCATGCCTCAGTTT
CCTCATCTTTCAATAATAAGGCCCACCACAAAAGGGCTGCTATGAGGAGTAAAGAAGCTAGTATGTGTAAGTCATTGAGACTAGCAC
CCGGAACCTAGTAAACGCTGTGTTAAGTATTTGTTAAATAAAATAACTAAAAAGCACGTGAAGGTGGCTGGCTCACAGGAGGCTGTG
GGGTGATGCGAGTGGATGCTGACGGGTTTGGCGGCGTGGGCTGTGCTCCGTGGAGTCAGTTCAGGAAGGCAGGGGAGTATTTGGTGC
CTCTCAGGAAGACTATGGTAATCTTCATGAAAATGAACCATGGATGCTTGAAGGAATGAACCCTCCATTGTCTGTTACACTGAATGC
CTCACACCCTGCATTTCCCAGTGTTCCTTGAAATATTAATAGCTGTTACTTCAAAACAAGAGTCAAGGTCAAATAAGTTTGAGAAAC
TGCATTTCAAAGCCGACTTGTTTTCTTTTCTTTAGAAGTCTCAGAAGCTTTAATATGCTAATGAGCCTGATGAATCTCTGAGAAGCA
TTTTTCAAACATTTGGCCACTCAACCACTTTGTATGGTGCTTCCAACATGGCCAGCACTCGGCAGAAGGCGCCTCAGAGCAAGCCTG
TATACCTGTGCCTGCTACAGAGGTGTGTGAAGGTGGGACAACCCATCTGAGAGGCAGTGTCAGGGGCCTGTTTGCCTGACCTCAGAA
CTAACTAGAAAATGCAGCTGCTGCTGCTCCTGTCATTCATATCCCAGACAGTCAGAAAGGGTTACCGACAGGCCCATCCATCCCTGT
TTCCTTCAGGGCCTGGGCCAGCCTCCAGTGGAGGCGCCTAGACCCTGAGGCCCCAGACCCTAGCATGCCCCTCTCTTGCCTCCCTCC
TGACCCAATCTAAGACCCTGAGGGCACTGTGGATGCCACAGCCCTCAGATCTAAACTTCATCCTGCCCCCTCCTCCAGCTACCCCTT
GGCTGCTTCTCAGGCTTAGGGATGTACCTACCAGCAGCCAGCGTCAGCCTATGGGAGGACAGAGAAAGTGGCAGCGTGGGCCCTGGA
AGCAGGCCTGGGGCCTTTTTGGACAGGGGACTCTGGGCGTCTGGATATCCAAACGGGGGTTTAGAAGGTGGAGGCAGACAAGGCCTC
TTGGCCCTGTGGGCTTCTGACTCCACGGGGTGTGGTGTGACTTAAGGAGGGGTCAAGCAGGGCCCTCTAGTGTGGGGGACCCAGAGC
CAGAGCTCCAGGTGTCTGGATCCAGGGCAGTACCTGTGGGCATTGAACACACAGCAGCACAGACCCCGAATTGCAAATAGTGCCCCC
AGTCACCAAACTGGCCCTCTCCATAGCCATATCTTTGTTTGCTTCTGCGCATCTCTAGCAGACACGCAGTTCTAAGCTCCTGGTTCA
TCACGAACTTGCATCCTGATCTCCGCTGTACCCCACGGGTCTTGGCATTGTACCTGGCACGCTGAGGCCAGATCCGCAGTGACCTCT
CACCAGCAGCAGTCGCTGTGGTGATGGTGGCTGCCGCATGCCCTGGCCCTGGCCCTGGCCCTGGCCCTGGTCCCTCCCTCCCAAATG
GAGGGAATGGTGGAGCAGGTTTCCTTTCTCAACCGTGGGGAGGGCCACCACTTCCCACAGGGAGCCTAGGCCTCTGCCCTCAGTGGG
GGAGCGGAGTGGGCCACTGACTAACGCGGCTTTCAGTGCCTACGATAGGAATTCACATGGAGAGGTGGCCTTGAAATAAGTGCCTTG
TTTAATCTTCATGCCCTGGAAGTATTTTTCACAAGGTGGCTTAGTTAGAGGGGCTGTGGATCTGGTAATGAGGCTGGCCTTGGAGGG
GGAGACTTGTGAGAGGAGCTCAGCTATGAACACTTCTAGCAGGGCTGTATTCATAAAATCCTGGGGGCTCCCAGGGGATTTGAAGCT
CTACACAGAAAATTACAGGGCAGGAAAGGACCTGGGCAATTATTTTTCCTCCAAGAATGGCCATGCTTAAAACATCACCATTAGGCG
CCACAAGTGGCTTTCTCCATGACAGCTCTTTTAAAAAATACTTTATTGGGTTTTTTTAAATTACAAAAGCAATATAGGCTTCTTTTA
ACACAGAGGTGTGTTCTCATTATCAAAAGGAAAAAAATCAATAGGTTCCCTGATTCCCATCTCCCTGCGTCCCCAGTAGTACCAGTG
TTAACAATTTAGTGTTTAGCCAGAGGTGATTAATGCACCTTTTGTGTGTGCTTATACAAAAATGTACCAACATGCATACACATATAT
GGGCATTTTTGTTTTTATGTGTTGGTGTTTGCTTTTTTAAAAAAGAGGAATCACACTATACCCATTATGCTGTAAGTTTCTCTTTTT
ACTTAGCAATATATCATGAACATTTTCTAAGTCAATACATATAGATCCACTTCATTTTTAAAAACAGCTACATAATGTTCCCCAGTA
TGAGTACCATAATATATTCAGCTGTTTCCCTATTGATGTGTATTCAGGTTGTTTCCAGGGTTTTTTAAAAAATTATAATAAGCAGTT
CTTTTAAAATATTCTTTGATTTCTTTTTCTATCTTGTGCATATGTTACAAAAGACTCTTTTGCCTTAGGAGGATTATAAAAATGGTG
AACAATTAAAATATTTCTTTACAGTCACATTGATATAGTTTTGACACCTTAAACCCTTTATGTATTCATTTGGGTGGCCCTGGGCGC
TGTGCCTGGCACTGTGGAGGACAGTGACCAAGGAGATGTCTGTCCTCAAGGACCCCTGCTTTAGTGAGGGTGCTGACAAGCAGGTGG
ACAGGAATGCGGCTCAGATGGACGATCAAGAAGGAGGTTTCGGCCAGAATGGAAACTTAATAGATGAGAGTGTGGCCTCTTGGTTTA
AATCCTGTCTCCACCACTTAGTTGCTATGAGGTCTGGGGGATGTTCACTTTTCTTATGGGGCTATTGTGAGACTCAAAGAGTTGATG
TAAAGAAAAGTGCTTAGGAGGCCAGGTGTGGTGGCTCATGCCTGTAATCCCAGCACTTTGGAAGGCCGAGGATGGCTGATCATATGA
GGCTAGGAGTTTGAGACCAGCCTGGCCAACATCGCAAAACCCCACCTCTACTAAAAATACAAAAATTAGCCGGGTGTGGTGGTGCAC
AGCTACTCGTGGTAGCTGGGTACTAATCCCAGCTACTCGGGAGGCTGAGGCAGGATAATTGCTTGAACCCAGGAGGCAGAGGTTGTA
GTGAGCCATAATCGTGCCACTGTACTCTAGCCTGGAAGACAGAGTGAGACTCTTGTCTCAAAAAAAAAAAAAAAAAAAAAAAAAAGA
AAAAGAAAAGTGCTTAGGCCCCAGCCAACAAGTTGGACTCACCAAATGGCTTGTTGCTGTTTGAAGTGCTGCCCCCGCCACTGGGAG
ATCAGGAAAAGCTCCCCCTGAAGGGGTGACAGTGCAGCTGGCCCTTGGGAAGGAGGTGAAGGGCCTTTTAGTCAGTTGGGGAAAAAA
CAAACACTCCCTGCCCACCACCACCACCTGGTCCTGACCTCAGTTCTTCAGCTCCCTAGGCTGTTCAAAGATCCAAAAACCTATTTA
TTTTTAGTTGCTAAAAATATCCCCTAAGCCAGGGTTAAAGAATTTCTCCCTTAGTGGTAAACAGGTGACTTAGAACCAATCAGGGCC
CAGCAGCCCCAGTGATGATGCTGTTCCTCCCCTACCCCCAAACATTCAGCAGGATCCTTTCATGTGTCAAAAGGAAAGAAACTTATC
AGAAACTTCCTGTTGAGAAAACGATACCTCTTACCCCAGACTTAGCAGATGCACAGAAACCTTTGACTCCTGGATCTGCCAAGGCAC
ACAAAGAACCAGAAACACCTATTGGCCTGGCCTGGGATAGGCAAACACTGGGGCTCAGCCCAACAAGCCTTCCTAGCACAGCCTGGG
GCAGCACACACAGTAGTCAAGAGAGCTGCCTGCCCTGGCTGTAGAGCCTAGGTCTGCCCCTTATTAACTGTATGACCTGTAGTAAAT
TATTTAGCCTCTCCGAGCCTCAGTTTCCCTGTCTGTAAAATAGAAATAAGAATACCTGTCTCAAAACCCTGTTAAGGACTAAATGAG
ATGGTGCCTGTAAGGCACACAGCACAGGACCTGGTGCACAGTGAGGCTGCAATACATGGCCCCTGCTGTTACCATTGTTAATGGAGA
CCGCCGCCCTGCTCCCTCCGCCCCCTGAAACGGGGGCTAGCACTCCCCAACTGTCTGGCCATCCTTCTCCAGCTGCCATTTGGGGAT
TGGCTCTGTGACAAGCAGGGGGCACAGGGCCTGGGGAATACTGGAGGAAGGCACCCAGCCCCACCTGGGAGTCAGAAGGGACCAGCT
TAGGCCTGTGGAAGGGTGGGACGCTGGATCTGGAGGTCTCTGGTGAAGAAGCTGAGAAGAGGGAGTGCTTGTACAGAAGATGGAAGG
GCGCAAGCTTAGGGGTTACTGGTAACTTGGTTTAGCTGGGATGAGACACCGGGTGTGGCAGGTGATGGGGATAGAGGGGCCAGCTGG
TGCCAGGTGGGAAAAGGCCCTTTTGGCCAAGTGCAGCTAGGAGGGTGGACACTCTGGGTTGCCACGGAAAGGTTCTAGAAGGGGAGG
TGACATGATTTGGTTTGTATTTTAGAACATTTGCTCTGGCAGCAATGTAAAGATGGCTGGGGTGGAGCACATGATGAGACCAAGGTG
TGGCCACAGCAACCCAAGGATTGGCCGGGGTCACTTGGCCTTACTGCTTCAAATATAATTGGTTACAAACTCCCACCCCACACAGGA
GGGGACCCGGTGTCTTTCCACTGGTCTCTGGGGGCTTGGAAACTTCCATTTCTCTCTTGACAGCTCATCTGATGCCAAACTCTTTCT
GCTGAATTCTCCACCTCCATCCTGTACTTAGTAACCGCTTTAGGCACCCTTCCTGTTCCTGTTGCTCCTCGAGGGACCCATGACCAA
GGCCTCTAGGTTAGCTGACATTTCCATGGATCTGGAGCAAGGGGAAGGGGTGAGGTGGTGGTGGCACAACAGATAAGACAGCGCTGA
ACGCCACATGCTCCTGATCTTACAGTCTCAGCACTGTCCCTGTCTCATGGGTGGTCAAGTCCTCAGTGGGCTCCGGCCTCCCCCTCT
CATGGGACTGGCCCAAGGTGCTGCTTAGCCAGCTCCCCTCTCCTTCCAGTCCCACAGCTCACAATCCCCCATCTCCAGTGTTCCTGT
TCATTTCTAGGGCACATATATTAAGCCCTGTGTTGGTCTCCTCTGACCCCACCCCAAGCCTGTCCAGAGGAATCTTCTTCATTAGCA
AACATGCTGGGGTGCATAAGTGGGCTCCTAAACTAAGATGGTCTCTTCCTCGCTAGGGACCCCATCACCAGGTCTCCATAGAGGCCT
GTTCACTGGCGGGAAGAGGAGGAAGGGAAAGATGATAAATGGATATTGTATCTGTGGAAATGAAAGCCGTGGTCTTAGTTCCACAAA
CATAGCCCAGTCTAGATTACAGATAACGATGTGGGTTTCCCAGGTGGTGTGGACACCAGGGATAGAACCCAGTTTATTTTAATCATA
TATTGATTCTCTGGTCAGCAAAGATTCTTCTGAGAATGAGCATTTAAAGGGTGGGCTTGAGTTTTCTTTCTTTTCTTTTTTCTTTTT
TTTTTTTTTGAGATGGAGTCTCGTTCTGTCACCCAGGCTGGAGTACAGTGGTGCGATCTCGACTCACTGCAAGCTCTGCCTCCCGGG
TTCAGGCCATTCTCCTGCCTCAGCCTCCCGAGTAGCTGGGACTACAGGCGCCCACCACCACGCCCGGCTAGTTTTTTTGTATTTTTA
GTAGAGATGGGGTTTCACCGTGTTAGCCAGGATGTTCTCAATCTCCTGACCTCATGATCCGCCCGCCTCGGCCTGCCAAAGCGCTGG
GATTACAGGCGTGAGCCTCCGCGCCCAGCCGAGTTTTCTTTAGAAGACAAAGTAAAGCCCAAACTGGCCAGCCCCCAAATAGAGTGC
CTGCTTAGCAGGCTGGCAGGTGAAAGGGCGTGGGACTTGGAAGACACAGAATGCTGCAAGCCCCAGGCAATTGATTGAACCTGTCTT
GGTTTGGGTTTCTTCATCTGCTAAATGGATGTATGACTCCCTTACAGGGACATTATGAGATGGCAATGAAATGGTATGTGCAGAATC
ACCTGGCTCGGCCCACAGTCTGGCTTTTGGGTAATTTTGACACAAAAGGAAATAGCTATGAACTGAATTGTGTCTCCCCAAAACGCA
TTTGTTGAGGCCCTAACCTCCAATGTAACTGCCTTGGAGAAAGGGTCTGTGGGAGGTAATTAAGTTTAAATAAGGTCATAAGGATGA
GGTCTAGTCCTATAGGACTGCAGCTCTAGGAGAAGAGAAAAAGAGAGATCCCTCTCCCTTTCTCTGCCATGTGAGGACACAGTGAGA
TGGCAGCCACCTGCAAGCCGGGAAGAGAGCCTGACCGTGTTGGCACCCTGATCTCAGACTTCCAGCCTCCAGAACTGTGAAAAAATA
CATTTCTGTTTTTTGAGTTACCCAGTCTATGATATTTTGTTTCGGAGGCCCGAGCTGTGGTGCATATTTAACCTGTGGGCAGTGTGT
TTTCAGAAAGTGGTGACATGGTGCACATGGCCCTGGTGGGAGTGGGTCTGTGGTCTCCTGTCCTTTGAGGAAGCTCCTTCTGTGACA
TCCAAGGTCCCAATGATCTGGGTCCTGCTACCTTCTTGGTCTCACCTGGTATCATCCCTCACTTCTTTCTCACATTGGTCTTCTTTC
TTTTATCTCTCCAACTCTCTAAGCTCCATCCTGCCTTGGGGTTTTTGCACATGCTGTCCCCTCTGTAATGCATACCTTTGCAAGGCT
CACTTTGGATTCAGAGGTCCCTTCCATAGAGAGCCCTCCCTGTCCTGGCTGTCTGGGACTTCTCTCATTTCCTTTGCCCTGTCTTAT
TCTCTTCCTGGCAGGCATCACTCTCTAAAATTAAATGGTTTACCTGTTTAATATCTGTTTTCCTCCCCTGAAATGTAAGCTCTGTGG
GGACAGGGGCCTTGTCTACTTGTCTTGCTATATCCCTAGAGCTTACCAAGGACTGTCACATAATGGGGGCTCAACTCATGCTCAATG
AATGAATGAGTGAATAAGCTGATCGGGGCAAAGCTCCCTGCCTGTGCATCGAGCACCTCTTGAACCAGACCCCAAACACTTGATTTC
CAGCATCACACCTTGTCTCTCCTTACAACTAGAGACCAGCTGGTCCATCCAAAGGATTTCCAGTCAAAGGGTTCTATGGAGAGCATG
GGGTCCACTACAGAGACCTAGGCTGGAGGAGGAGGTGAGTGAGTTGATCTTTGAATGTTCTTCTCTACCCTTTCAACTACAACAGCT
CTGAATTTGTCTGTTTCCCATGTTGGGCGTACATGAAAAATCTTATGAAAGAAATGGCCCCACAGCCCAGAGAAAATGGAAAACCAC
AAATTGCATCCAAGTCTTCACATTACACTGAGGAATCTGAGGTCCAGAGACTTGCCACACACAGATGGCCAGAGGTGGGATGCTCTA
TCCCTAGTCCTGACGTGCTCAGGGATCAAGAAATTCCTGCTACGTGAGTAAAAAATAGTGGCTCGCTCAAATGCCGAAACTTGGCTT
TCTTTCTCTGAGAACTGAGGACAGGGGAAAGGATTTATGATCACCACTGGTCTCTGTTAGTCCCAAACAAACTTCAGACTCCATAAC
CCCCATGAGTGGTCATTACACACTGCAAGAGACCAAAAGTGAGCTCTGGCGAAGCGCTCAGGAGAAGCCTATCCTGGCCGGACCAAA
GAGCCACTGTGGGACATCACACATGGCCCAGGGAGTGGGATGGTTTCAGATGAGGACAATTTCTTCTTAGACACAGAGCGGGGTAGC
GTGGACCCCAACGCCCTAAGCATCTTGTTTATGGTAGAATCATAGACTTCCCTGACAAGGCCTGTTCACTGGCTGGCCTCTTTTAGA
GCCACCATTTTTCCACCTTTGCTTCCCAACCCCCACCCGCCTCCCTTTCTTCCTCTAGTTCCATGGAGCATTTGCTGACTGGTAATT
TTACCAACCATTGCTGCAAACCCAGAAGTTCATTTCGTCACTGGCAATCAGCTCCTGTCCGCAGGGAAACCTAATTAGGTACTACAG
GATTTTTGATGTTGCTTCCATGGTAACCCCCGTAGTAATGGTGTACATGCAGAATATATGCTCCAGGGTGTTTAAATTTATATATGT
ATATATATATATATAAAATATAAAACATATTGTATATATAAATAATATATCTTAAGAAAATCTGTAATTTTTTCTTCTTGCTTTGTT
TCTGTGTCGTTTTTTTAAATGATCGAAATTTTCCCGGAAGTTCTTCTCCCCACAGAGAGAGAACAGTACACAAAAAGGCTTTTTCCA
TTTTCTCTCCCTTAATTTAGAGAAATTCACAGTTGTGCAAGAGTGAAAGTTTTTCAGGTTGGTCAGAAAGTGTCTAAGCAGGAATAC
GCTCTCCTCCCTGATAGCTGAGAACATTTTAATGACAGATTTTGCTTAAATAAAAAATCAGAAAGGTCTCAAGTCTTTCAAAATAGA
ATGGGGGGGAAAACGAGTGAATCCAATGAATCGACTCTAAACTTATCCTAAAAAAAAAAAAAAAAAAAAAAAAAGGAAAAAAAGAAA
AAAGGCAAGCTTCCCTGCATGCCTACCCCTTTGCAGGAAATGTGGGGAGGCCTCGCAAGTCTGAAGCTCGGGCTGTGCCATGTGGCT
GCTGGAGGCACTGGGTAGTGTCCTTGTCTCCTTGTCAAGGCAGCCTGTCCCAGGCCCTGCAGAGATTGCAGCTGAACGGCCCTGTCC
TGAGGTGTCCAGATGGCCTTCAGCGTTGGCCTTGTGGTGAGCTGGTGATGACATCTTTGCTTGGCGAGACACTCTAACCAGGGAAAG
GTCAGACCTGGACTCCCTGAGGTGCAGGTGACAAGGGAGAGGGCCTGTGACACACCCACAGCTGAAAGCCAAGGGCTTCTGAAGAAT
CCCTGAATAACTGAAAGTGCTTTCTGTACTATCCCTCGCTGGCCGTCCAAGGATGGAATGCCATGCTGACCTCTTTGTTAGCTGCCA
GCGAATCCATTTACCCTCAGCATTTGCCAACAAGGTCCACTGCTCCCAGACTCCCTGCTGTTTTTCTCCAGCGGCCCCCCTGCCCAT
TAATTCTGTTGTGCCCTTGTTCCCTGATTCAGTCTACCAGCTGACTTCCTCACATCTGAGAAAACACTTTGTATGCAAAACAACTAA
ACTGCTTGCAAAATAACTTTTTAAATGCTCTGAGTTATTCTTTGATATCTCAAATCATGTGTCCATGGGGAGAGTGGGGATGGGGGA
ATGGCCATCAAGGAATGGCCCTTGACCCTGATCTCCTCCCCATCCAGTCACCCAGTTACTTGAGAAGGACTGGCCAGCAAATATAAC
TGAGTGGAGTGGGAAGGTGGGGACGGGTGAGGGTTCCTGCTAGGGGCTGTGGCATAAGAATCTAAAAGGAAAATGTTGGGCACAGTA
GGCACATGACCTGGCTTTTCTAATATGTGCCAAATATAAGAATTAGTTTTGTTATATATTAGTTTTGTTGCATAACAAAGCACCTCA
AAACATAATGGCTTAAAATAATAATTTAGGTCACGTTTCTGTGGGTCAGTCATTTGGGCTGGGCTCAGCTTGGTGGTTCTTCTGCTG
GTCTCACCTTGGGTGAGCTTCTGTCATGCAGCTGGTCTAGGACGGCTTCTGATGGCTGGCAGCTGGTACTGGCTGCAGGCTGGGGTG
AAAGGGATAGTGAGGCCATGTGTCTATAGTGAGCCTCATGGCGATGACATTTTTTAAACCTCTGCTGTATCACACTTTTTAATGTTC
CACTGGCCAAAGCAAGGTACATGGCTAAGTTCAGACGGAAGGGATTGAGAAATAGGCTGTACTGGCCAGGCGTGGTGGCTCATACGT
GTAATCCCAGCACTTTGGGAGGCCAAGGTGGGTGGATCACTGGAGGTCAGGAGTTCGAGACCAGCCTGGCCAACATGGTGAAACCCC
ATCTCTACTAAAAATACAAAAATTAGCCAAGTGTGGTTGTGGGTGCCTGTTATCCCAGTTACTCAGGAGGCTGAGGCAGGAGAATCG
CTTGAACCCGGGAGGCAGATGGTGCCATTGCACTCCAGCCTGGGTGACAGGAGTGAACTCTGTCTCAAAAAAAAAAAAAAAAAAAAA
AAAAAGAGACAGAGAGAGAGGGAGAGAGAAATAGGCTGTACTTCTTGATTGGAAGAGAGGCAAAGTCACATGTACAGAAGTAAGTGA
ATGGGATGGGAGGAATCTGTGGCCTTTTCTGCAAGCTGCCCCCTCTGGTGTGTCTGATGCATGCCTTCCGGGCGCTGCTGAAGCCCT
ATGTAACCCAAGAAGCTTTTCAAATTGCATCCTAATCATGCATGTCTTTTGCTAGAACTACTTGAGATTGTGCACTGATTCATGGGA
CATGTGTTTGATTCAGATTTATATACCCACAATGGCCTAACCTTGTTGATTCTTAATAATTATTATATTGAACTGAGTTGGGTTAGT
TCTCCTTTTTCTCTAATTACAAGCTCCTTAAAGGAAAGTTTGGAATCACTGTTGCATAAAATCTCCCTCCCAGAGCTCTCAGAATTA
TACTAACGGCTGAAAATCCGGCAAGTTCTCAGAACCACCAGTTAAGTGGTAGAGGGATTAGGCCTAGAGCCACCTCTGGACAGAGCA
ACTATCTGAGGAAAGAATGGAATTTTCAGGAAACCAGAGACTGTGGTACTGACACATTGCATCTTCAGGTCAGTCATACCCTCAATG
AGTGAAATCACTTTTTTAGGCTTAGGAGGCAGAAAGAGTCAGTGACTCGAAGAATGTGGCTTTCCTTTTCCCATTCAGGGAAGCCAG
AGTACAAGGAGCAAACCTATGCCTCATCAGGAAGAGACTCGTTTGTCAGCCCTGGGCCTCTGTGGCTTCTCCTGCTCTGGAACAGGC
CAGAACAGACCACTTGAGGGCCCACGGTACTCCCTGCCCCATGCCACACTCTGTCCACATCACCTCAGTACTTCTCCAGAATCCATC
AGAGGCCAACAGTCAAGCTGGTCTAGAATCAGGGCTGGATGACTGAGACTAAGCCCTAGGGGAGGATAGTCCTTTGTGGAGCCACAT
ACACACTCCCCGAGCCCCTCTGGTCCAGTGCTACTCAAGATCCTGGTTCCAGACTTGCTCTAGAACTTTCTAAGGCTTGGAGGTCAC
CTGGGACAGTTTATTCCAGGTTGAACCACCCTTTGGGAATAAGAGGGTGTCCCTTCCCATGGTGTGAGATGAATTGTCAGGTCCTTT
GTGAGTCACAGGCTTAGGAATGGATTTTCAACAGGAATTTCCATTCTGGGTGGTTAACAAGGGCCACAGGCAATCTGTCCTTTTCTT
CATATGACACAGTCATCCGCCTACACACCCCTTTATTCACCAAGATTCTACTGACTGCCCACTCTGTGCCAGGCAACGTCTCAGGCA
TGGGAGTGGGGTGAGATGAGCCAGGCACGGTTCCTGTCCCTGGAGAGCTCAGGTGCACCGAGAACACAGATGAGGCAGGAGGTGCGA
GCAAGAGGGTCTGTGTAGAACCACAGAATTATGGCTTCTTAGAGCAGGAGGTACCTTGGGGATTGTCTAGTCTATTCCCTCATTCCT
CAGAGGAGGACGCTGAGCCCAAGAGAGGGAAGATTAGTCCAGGGGCACGCAGTAAGTGGGAGTTACTAATTCACTAAATAAGCATTT
ATTGAACACCTACTAAGTGCTAAGATCTGGGCTAGGCTCTGGAGATGCCACAGGGAACAAGGAAGACACAGCGCTTGTCTCTTTGGA
GCTCCAACTGTGACTGGTTGGGGGGTCCCAACCCTGGTTACATTTTAGACTCACCTGGGAGCCTTAAGAAGAACACCCGTGCCAGGG
CTCCACCCCCAGAGATTCTGATTACTTGGCCTGGGGTGGGCCTGGGCCTGGGCATGGGTATTTTTGAACAAGCCCTCCAAGTCATTC
TAATGTGCGACGCAAGTAGGTTGAGAACCATGTCTAAGGGGAGAGACAGCAAAACCTCACACATGCAAATCAATAATTGGGATTGTG
CTTTGTTAGGAATGAATGGGACCCAGAGTGAGGAAACAGCAGGGTTGGGGTATCTGCTACTTGCTCTGAATCGGGAGGGAGCGAGGC
CACACTTGGAGAGCTGTGATTCCAAGGGTCAGAAGGAAGGAAGCATAGAGTGTCCTCACGTGGGACAGCAATGGGATCTTCTGGGAC
ACATCCCACCCTGCAGATGCAGAAAACCTTGCAGCGGAGCCCTGTCTAGCAGAAGCTTTTAACCTGCAGCCTGAAAACTCTCCCCCA
CCCCCTACTCAAAACACCCGCTCTTTACACACACTGTTTACGAGTGCCCCCAGGCCAGCCAGCTTAATAATCCCTCACATTTTTAGA
GCAGTGAGTAAAGCACTGTCCAACTTTTTATCTGTATTATTGGATCCTAACACTAACCCCAGGAGGCAGGCAGGGCCAAGGTCAGTC
AGTGTTCCCCTGTTGGAGATGTGGAAACAAGCTCGGAGAAGTCACGCACGGCCTGAGATCACATGGCTAATACCTGGATGTGGGGGA
CTTCAGGCTAGCTCTCCCACCTCCAGCTCCACTACCCCATGTGGCCCCCACCTTCCCTGCACCAGCAAGCCACAACCAAGGCGGGGC
ACCTGAGAGGCAGCTGGGGACCAGCACCCTCCCGTCTCGCTGGGCAGAGCTGCCGGTGCATTCTCCCCTTCAGCTCTTACTTACAAT
CCCACTCCTCATCAGGCAATCAAAGGAATCTGTTACTCGTTGCTTCCATTCCTGCCCAGAGTGAACCCAGTTACTATAGTAACCAGA
TGCGATTCTTTATTACGGAGTAGATGGTTTTAATTTTGTATTTGAAGAGTTTGGCCAGGCAGTCACGTTGCCATGGCTTGGCCTGTC
GGGCGGCAGTTAGAAGCAGAGGGAACCCTTTGAGCAGCCATTAGGGCTTTCTAATTGCTGGGGAAAACCTATGTCTGGAAAAACCCA
GGCTCAGCTGCACTGGGGAAGGAGGAGGGTGAGTCTCCAGCTGGTGCTGTGCTGGGTACTGTCTCCTGTTCAGAGATGACAGCTCCC
TGATTAGAGCCACCAGGTGAGCCTCTCAGCCAGTTGCTCCCATAGTGCCCTGTAGTTTCCCTTCAGGATCCTCATGGGTGGGTGGGT
GGGGGGCTGGCTGCTCAGAGGGCCATCAACACATTTTTGTTGAATGAATGAATGAATGAACAAATGATCCTTTGTAGTTTATTAAAC
CCTTTCAATCCATTGTCTCCTCTGCTCTCCAGTATGGCTCTGTAAGGGTACGGAGCAGGGAGAAAGAGAAACTGAGGCTCGGCAAGG
TAAGAGACTTGCCTCCTAGTGCTGCCTCCCCTGTCTGTGCCTCTCAGGGCCCATGAATTGCCTGGAATGTTCCCCTTGCTGCAGAAA
CATATCACAGGTAGATGCTAAAAGCAAGGCAAGCATCGCCCGCCCACACCACACCAGGCCTGGTACCATAGGTCCTCTCCTGGGTCC
TCAGTTCTTGCTGAACTGTAAGGAAGGCAGCAAAGTGGAGTGAGAAAGCTGCTTGGTGACTAACTTTGAAGCAGAGCAGAAGCTTCC
TTTGGCCCAGACCATCAAGGAGACAGTTGGGTGGACAGAATAAAGGGCTCCTCATTCCCTGATCTCCCTGTGCAAGGGAGGAAAGGA
AGGCTTCACTCGGCTTTCAGCCAGCCCCCGCAAATCCACGCTGATCCAGTCTCTCTGTTACGCTTTCTGTGCTGAGGGTGTATTTCC
AGGTTAAATAAAACCTCCACCCAACCCTGGCCCCAACATTAATTTCTCCTAACCCAGCCTAGTTTCTTCCGGCCTTGCAGCTCAGGC
CTTCCCAGGATGCATGCATCGCCTGCTCTCCCAGCCAGGAGGTGCTGATGCTGGTACCCTTCATCCTGAGCTCATGTTAGGCCACAG
GACCCTGCTAGCTCCTCCTTAAAGGCAATCATAACCAGCTTTGGAGAGCAGCAGCTCTCTGTGGCTGTTGGTGGAGCAGGCAGCATG
TGAGGCTGGAGTCTTGAGTGGGATGTGTTCGTGGATGAGCTGGGACAGGCAACAGCCAGATCTGCTGGAGGGCCAAGGACCCAGTAA
AGAGAGGGCATAGTGTGATGATAAACATTCCACTACCTCCCAGAAGTGAGGTGCCTGCACAGGAGGCCCACACCGAACATATGCCCG
GATCTCAGGCCTCACCCTCCAAGAATCAAATCATCGGAGCTGGATGAGACTTTAGAGGCTTCTCATTTCAGTCTCAGATGAAGACTG
AGGCCCCAGAGGGGAAGGATGTGAAGTCCCTTTCACAGTCATCAAATATGGTGGGACTCCACAGTGGGACTTACCGGAAGACAAAGG
CTGGACACACAGCAGGTGCTCAGTCAATTCTTGTGAGAACAGCCATCCTCATGAGGTGACAGCAGGAAGCATCTTCGTGAAAGTTTA
GGGCCAGAAGGAAGTTTAGACATCACTGAGCCCAACTGTCATTTTACAGAAAGACCGAGACCAGATCCATTAATAATGCTACAAATA
AGAATAGCTAATAGTTACATAGTGTGCTGGGTGCTAGGCAGGATTCCATGCATGTTGCATATACGAACCCACTGCAGCCTCACAGTG
ACCTCTATGCAGCAGTTATTATCATCATCCCCAGATGAGGTAACTGAGGCACAGGGTGGTTCGGTAATTTGCCCAGGATCATGGAGC
CAGTAGTGTTAGGATCAGGCAATCTGGCTCCAGAGTCCGTGCCGTGAACACTTCACTGCACTGTCTGAGAGCACAGTGCTGCCAGCA
AACACTTGCTGCCCCCTTCTCTGAGTGGGGAGAAGGAATCTGTGCCAGCCCTTGTGAGTTCACTCATGCAGGAAACATGACTTTTTC
ATCTTTACATCCCCTTACAGGGCTCAGCTCAGGTCTGGCAGGGGCTCAGGGATGTGTTTCTAATATAAATGAGTTGATCTTTCTCAC
CATCGTGAAACAGCCAATTTGGCACCTGTGTGCTGGAAGGGACCTTGGGAAGTGGCTCAACATAGAACACTTGTGGTCAGCAAAAAG
GAGGGGGTGGTTCCCACACACCAAGCAATTCTCCAATGGACACTGGCTGAGTGTCCTGCAATATAACTCAAGCCTGATTGTCCGCCT
GGAGTTAGTGTTGGCTCCCACAGGTTGAGGGCTCAGTCCCACCACATTTACCTGACTTCCGATGCTAGTCACCAGCCCCAGGTTGTT
TTATCTGAGCCTCTGACCAAGTGGCTATAAATCAGGGATTCCCAGGACGCCCTTCTCAGGTTTTCATTAATTTTCTAGAGCAGCTCA
CAGAACTCAGGGAAACACATTTACTGGTTTATTATAAAGGACCTTACAAAGGATTCAGATGAGCAGCCATATGGGAGCGAAGCAAAG
GGCAGGGCGCATGGGAAGGGGTGGAGCTTCCATGCCCTCTCTGGGCACGCAACACTCCACGTGTCCAGGGACCTCCATGTGTGCAGC
TATCTGGAAGCTCATCGAAACACAAAGAGGGGAACGACAGACACTGAGGGTGGAGAGTGGGAGGGAGAGGATCAGAAAAAATAACTA
TTGGGTACTGTACCCAATAGTACCTGGGTGACGAAATCATCTGCACAACAAATCTCTGTAACACGAGTTTACCTATATAGCAAACTT
GCACATGTACCCCTGAACCTAAAATAAAAGTTAAAAAAACAAAACAAAACACTGTCTTTTTGGGATTTTGTGGAGGTTTCATTACAC
CGACATAATTGATGACATCATTGGCCATTGGTGATCAACTCAACCTTCGGGCCCCTCTTCCTTCTCAGGAGGTGGTGGGGTGGGGCT
GAAAGTTCCAACCCTCTCATCACCAGATTGGTTCCCCTGGCAACCAGCGCCATCCTGAGGCCACCCTGGATCCCCACCCCGACCGAC
CCCAGCCACTAGTCATCTCATTAGCATACAAATGACACTTATCACTTTGGAAATTCCGAGCGTTTTGGGAACTGTGCCTAAACCAGG
ATAAAGACCAAGTACATTATTTCTTATTATAAATCACTATATCTCAGGCAGCGATTTGCAGAGGAGGAAAGTGAGGCCAAGAGAGTC
AAAGTGTCTTAGCCAGGGCTTCCCAGGTAGACTGAATCCAGCTCTCCCTACTCCACATACAGGGCTCTTGCCACATAGCAAGGCCCC
TAAAGAGCATGCTGAGGTGGTCAGGCTGACCTGAGCCCAGCAGCCTGCCCCAGCCTGCCCCACCTGTCCTGCCTGGACCCTGCTGTG
GGCTTCCTGGTCCTGGCCACACTAATGGACTTTCTGCTCCCACCAGCTCTTCGTGATCGACAACGGCGCGCATGACTGGCGGATAGC
CATGACCTACGAGCGCATCCTCTACATCAGCCTGGAGATGCTGGTGTGCGCCATCCACCCCATTCCTGGCGAGTACAAGTTCTTCTG
GACGGCACGCCTGGCCTTCTCCTACACACCCTCCCGGGCGGAGGCCGATGTGGACATCATCCTGTCTATCCCCATGTTCCTGCGCCT
GTACCTGATCGCCCGAGTCATGCTGCTGCACAGCAAGCTCTTCACCGATGCCTCGTCCCGCAGCATCGGGGCCCTCAACAAGATCAA
CTTCAACACCCGCTTTGTCATGAAGACGCTCATGACCATCTGCCCTGGCACTGTGCTGCTCGTGTTCAGCATCTCTCTGTGGATCAT
TGCTGCCTGGACCGTCCGTGTCTGTGAAACGTATTCCACATGGGGCTTTCTGAGTACAGAGCCTGTGCTGGGACAGGGAAGGGAGGA
GGAGTAGGGGTGCCAGGAAGTATGTGGTCTGATGGAGGAGACAGGCATGTACCCAGACACCTGAAAGGAGACAATGGGAAAGAGGGG
AGTGCTGAGTTGGGGTTCAGTATGAGCTAGAATTGCAGCATTGGAACCAAAGTAAGGTGGGAAAAAGGCACAGAATTTTCCTGATTT
TCTGTTCCTTCTTTGCAAACCAATATATCAAAGCATTCTGGTTACATCAGTAACTCACATATAACACATTGCAACTTACTCCTCTTG
GGTATTAATAATGCTTATTAGCATTTTAAAGGCTCTGAGAAGTCCTGAAGTAATGAAACCATTTATTTTTGTTTGGTTCAGCCTTTC
TTAATTGTATTTGCCTGCAAATCCTTTTTATTATAAAGCACTATTGGTACCTTGCTACTCAAAGTTTGGTTCACAGACCAGCAGCAC
TGGCATCACTGGGAACTTGTTAGAGTTCCCAGAATCTCAGGCCTTAGCCCAGAATGAATGAATCAGAATTTGCATTTAACAAGGTCA
CCTGGTGATTCATATGCACCTTAGTTTGAGAAGCACTGTACTGATGGAACATACTTTGGGAAACATTGTTTTAGGAGGCTGGTGGTT
GGGAGTCATTCAGAGAAGCTACTCATGGGAAAGAAGGGAGATGTGGCCTGACACAGATCCAGGGCTGTAGAACAAAATGCCAACTCC
TTGGAACAGCCATCAACAAGGCTCAAGGGCAGGACTGAAGCCACCTCCTTCCACCACCCTCGAGCTGCCTTTAATGACTCTTCTCCT
TGCAAGGGGTCCTGGATTTTCATTTTATTTATTTATTTATTTATTTATTTATTTATTTATTTATTTATTTATCTGAGATGGAGTCTC
GCTCTCTCGCCCAGGCTGGAGTGCAGTGGCATAGTCTCTGCTCACTGCAACCTCTGCCTCCTGTGTTCAAGCAATTCTCCTGCCTCA
GCCTCCCATGTAGCTGAGATTACAGGCATGCGCCACCATACCAGGCTAATTTTTGTGTTTTTAGTAGAGATGGGGTTTCACCATGTT
GGCCAGGCTGGTCTTGAACTCCTGGCTTCAAGTGATCCTCCTACCTTGGCCTCCCAAAATTCTGGGATTACAGGCGTGAGCCAGCTG
CACCCAGCCCTGGATATTCATTTTAATCTATTTGACATGTGGAGTCTAAATCTAGGTTAGAAACCGTGATTCTGCTCCTAAGTAGCT
GTGAGACCTAGGGCAAGTTACTTAACCTCTCTGGGCTACAGTTTCCTCATTTTAAAATGAGGATCACTTGAGATAGAATGAAGCTAT
GTTTGCTTGAGATAGATTATTTTTATTATGTTGGGAGGTATGGTGAAGCATCCTGTGTAATTTACACAAATCCTGGTAGTCGCCCTT
AATTTGTCAACACTTACGATTATATTGCCAAATCTTCCATGCACCAGTGTCCTAGCAGAGATCAAACAAGACAAAGAAGTCTGGAGA
GGAGTTAGCCTCAACACACCCTACATGGGTGCCTTGAATCGTATCTCCTACCTATTTTTGAGACATTCTGTTTGCAGACATTTCTTT
CAGCGTTTTGGCCAACAGACACACGGTCAAACTGAGAGGGCATTCTGCTGCCGCAGAACAAGGGTTCAGGCTCCAGGATGCAGGTTC
AGGTTCTGAGAGCCAGTGCAGTGTTGGAAGACCTTTCTCACACACAGAGCCCCAGGGACACACCATGCTCACCTATTCAGAACCTCG
CCATTACTCAGCAGCTGACATTGCAACATCTGCTCTGAGTGGGACCACACTGCACAGGGCTTCATGAGCCTTCTCTGGTAAGCAGAC
ACTCTTCCCCCAATGGAGAAAGCATGTGCTTGGGTTCCGACCCCAAACTCTTCATGGTACTGTTAGCTGAGCAACATTGTGCTGGTC
GCTTCACCTCTCTGGACCTCAATTTTCTTATCTGAAAGCTGGACACAGGATTTATTGTTAGAGTCAAACAAGGTGTATATATGCACT
ATGTACTGCAATCCAATGATGGTGAAAGTTAATAATAATAGTGCAGATCCCTTGATAACTCTTGGGAATTTTGTGCTGCCTTCTTTT
AATTAGAACAGCCCAACTTGTACCATACATGGGCCTATACTCTTCCTGTTTTCCTTTCTAAATGATGGAAGCCATGTTGTCTATTAA
CATAGTAATTGAATTTGCGTAGCCCTTTAAGCTATTTGAAGTCTTTCACTTCCTCAGATGCTCTCTGGCATGTATTTCAGATGGCTG
AACATGCAACAAAGTACCTGCAAGTGAGTTTCGTAGTTTATTGAACCTATCACAAGAAGGGGTGACTATTTTTCACTGGAAAATAAG
CTTGTTTTAAAGAATGTAGTTTGCAAAGTACTTAGATGATGCACCTTATTTGATATTGCATGGCCTTCCATAGCTTCCAAGCTGAGG
CTGGGTTAGTTTGGTCCCTGGTGGGCCACTGGGTCTCTCTCTCTCTCTCTGCAGACATGGGTCACAAGCCCCCGCTAAGACCAAGGC
ACAGAGGAACAATGAGCAAATCGGAAATTCTCTGAAAGTTCCCTTCTCTTGCGAAAAGGCCATTTGACCAAAGACTTAGGTATAACA
TGAGGTGTATTAAAGCATGGTGGATGTTATTGTTGCACCCCTGTCCTAGTTCTGTGTAAAATCCATATGTCATCCCAGCTTGCCCTC
ACAATAGATCTATGAGAGAGGTACCTTGCCTGAGGGAAGGAGAGCCCGGGTCCTGGAGGGGCATTTGGGTGAAGCAACATGTCATCC
ACTACATGGATGTGTTTGAATTGGACATGAAGGCTCATGGGAGAGTTGAGGAAACTCATAGAAAGTAACTGGCCCGAGGTCACAGAG
CTATTAATAATAAGCGTGGAGCCAAAATCTGAACTGGTTTCTGACTCTGAACTTCCATGTGTGTGTCACCAACCCAGGCTGAATCCT
GCACATACGAGGAAATCTTGGCCTCTACTCTACTGGCAACTTTCCCAATCTCAGAGTCTTAAGGCCACTGAGACTAACATGTGAATC
CCAGTCTTGCTGTTTTCTCTGCCTCTGCTGCCTATGGTCTTGACCATGTTATCATACTAGCCTCCTGGGCTGCCTTCATGTCCAATT
CAAACACATCCATGTAGTGGATGACATGTTGCTTCATGTCAACATGAAGGGGACAGATGCCCCCTCCAGGACCCGGGCTCTCCTTCC
CTTAGCTGGGGTTGTGACTGAGTCCCTCTCTTCAGGTCAAACCCCTTGCCTGGGGGCAGCAAGCTCCCGAGGGGATTTGTCAGTGTG
GGAGTTCAAAGGCCTAACCCCGTTTGCTTCTAGTAGGACACTCAGCCAGGCTCCAGGGTCCCTGAGGGATCGCCTAAGGCCTCTGTT
ACCACTGCATCCGGGAACTTCCTCTATACCCCAGAGGTATTGATCCTGAGAGCAGCCCCAGTAAAACTCCCACCCCCAAATCTCAGC
ATCTCACAGACTGTTTCCCAGGAATCTGCCCCGTTAATATATTGTTTCCTAAACAGGCAAAGCACTTCTCTTACCAAGACTAAGCAC
TATTTTTAGAATCAAGCTTAAATGATCATTCTGTTTCCCAATGTCAAATAATAAGTAAGATTGGCAGAGGCCTTCAGCCAGGACTTA
TTCAGCCAGAGACTGGACAGAAGCTGGAAGCAGGGGAAGCTTTGGGGAACTGAAGCGTCAGACCAGGGTAGTGTGGACAGGATGCCT
TGATCTCTCTCAGCCTGATCTAATGATGATTCTATCACCTTAGGCATCAGAGAGGGTAAGTGACTTGCCCAGGGTCACACAGCAGCA
TGACAGGTTCTAGAAAAATCCACAGCTGCTGATGCCCAGAGCCTGTTCACATTCCAGGTAGCTTTCATCAGCCTGGACTTATCTCTA
GTTGTATACACACACTTCTGAAGTTGCTATGGAAATCTCTCTACAGCAAGATGTATTTTAAAAGACAGTTCTCATATCTTGGGTTGG
TTTTTATGTGACAACAAAGGTTTTTCATATTTCCTCCTCCCAAGGAGAGAAAGTGGGATCATAATTACGCAGAACAGCCGTGCCATG
GGGAGGGCAGCGCCTGGTTGCTGGGAGGGCAGTGACAACAGTGGGCTTGTTTTCCTTGGCCCCCAGAGAGGCATTACATCATGGGTT
CCGAGGCCTGACAGTTGTGGCACTCTGCATTCTCTGGGATTCTCCTGGCTGCCTGCCCACCTCCCTCAGGGAGGAACCTCCTGATTT
TACACTGTCAAGTGCCAAGCAGCTTTACTGGTGGAGAGACCATCTGAGGCCCCGGGTCCTCACTGCGGCAGCAGGTTGGAGAGCCAG
CCTTCCTAGCGTGCCTTGAGCAGTGCAGTGGCCTGCCAGCTCACCTTTGTATGTTTATCTCCTTGGGAGGAGCAACAAGGCCTCCCA
TAACATTGATGAAGTGGGGTGAGCCTTGCTGTCACAGCCATGCTTTATAAATGACCTTACTGTCCTCTTGAGTGGATTCCCTTCGGA
CCCTTTGGACCCTTCACTTTGGTCCCTCCTGAGGCTCCAAATTCTGCAACAGGCTGTCTCAGTGTCCCAGGCCTTCCAGTCTGTCAA
CAGGTTTTTTATTTTGCTGGTTAACTTTTGGCATACTGATATTTTAATGAGAAATTTTATTCTGGAGATCTGGCTCCTAAAGCAGGA
GAAAAACAATTTGTGGCCCATTCTTTCTCTCATTAAGAGAGCCACCACTCCCATTGTAAGGAGATAAAAAGAGGCTTCCTTGCTGTA
CTCTCTTCTGCCCCTGCCTCAGACACCCAGGCCTGCACTTGCCAGCAGCAGGAAGCAAGACCACAGCTCCCTCCTCCTCCCATCTCC
TTCCCACAACCTCCTACCCCCTCCAAGAAGGGGTACTTCCACTCCAAGAAAACAGTAGGAATGGCCAGGCTCGGTGGCTCACGCCTG
TAATCCCAGCACTTTGGGAGGCCGAGGCGGGCAGATCACGAGGTCAGGAGATCGAGACCATCCTGGCTAACACGGTGAAACCCCCGT
CTCTACTAAAAATACAAAAAAATTAGCCGGGCATGGTGGCACACACCTGTAGTCCCAGCTACTCGGGAGGCTGGGACAGGAGAATGG
CGTGAACCCAGGAGGCGGAGCTTGCAGTGAGCCGAGATCACGCCACTGCACTCCAGCCTGGGTGACAGAGCCAGACTCCATCTCAAA
AAAAAAAAAACAAACCCCAAAACAAAACAGTAGGAAAGTGGTCATTACAGGGATGAAGCACCCTACATGGGGTTGATGGAAGCCTGC
AAGCTAAACCCTGTAGGCTGAGCAGAACTGAGCAGAGCCTGGGAACAGTGGGGCCAGGCACTGTGGTTCCTCATGGACTGCTTTGGT
TTGAGTTATAACCTCCAATGTGGGGCACACGCACAGTGATCCATGGGACTATTGGGACAAATGATCAGAACTTCTACATATATATAT
ATATATATATATATATATAAAGCCTGGCTAGTATTTAATAAATGCATAGAGAGCAATACATGTCTGTAATTTATAATAAATATATGT
ATAATGGAGGAGGGCATGCTCAAAAAATTTTTAATTGGTAGGAGCCCATGATTTTAAAAGTTTGCAAACCACTGGTTTATGATATCA
GAGGACTAGTTAGAGAGGATAGGAAAGTAGCCTACCACATGGGATGAAGCCTGCTAGACCCAGGAACCCTGCCCTCTAGCTGGAGAA
GTGAGAAGTTAGCCTCCTGTAGGGAAGGAGCAGCCTGTAGCGATCCCCTTCTGGTGCCAGGCGGAGAGATAGATGGCTGGGGAGGGG
GAAGGTGGGGAGGCGACAGGGATCCTGCTGGAGGCCTTTGAGAAAAACCTGCAGCATTAAGGAAGAGGAAAATAATCCTGAAGATTC
CTGAAAAATGTTTAATTATGAAAAACAAAAAGCAACATTAGAAGAGTCTTGGGTGGCCAGGCACAGTGGCTCACGCCTGTAATCCCA
ACACTTTGGGAGGTCAAAGCAGGTGGATCGCTTGAGGTCAGGAGTTGGAGACCAGCCTGACCAACACAGCAAAAACCCATCTCTGCC
AAAAACAAAACAAAACAAAACAAAAAAAAACCAAAAAAAACATTAGCCGGGAGTGGTGGTGCATCTGTAGACTCAGGAGGCTGAGGT
GGGGGGATCACCTAACCTGGGAGGTCAAGAGTGCAGCGAGCAGAGATTGCGCCACTGCACTCCAGCCTGGGTGACAGAGTGAGACCC
TGTCTCAAAAAAAAAAAAAAAAAAGGAAAAAGAAAGAAGAGTCTTGGGCATCTCTAATAGGGTGCCAGAATATATATGATCCTTATG
ATAGACAAGAGTGGGCAGATGTGAAATGAAAATGTATCTCGTTCACATAAGCACAGAGTTCCAGAAAACTAAAATTGTTTTAGCAAA
ATGAAAATCCACATTGATGGCACTACATTTTTAAAAATGTGGAATGTGGAGACAAACTTAAAAAATTTAAAGCTCTCTCAGAATGCA
GAAGAGATAAGAGAAAGGATGAATAGGACAGAACTGGAGGCTATAGGATCCAGGTGAAATCTAAGAATTAGAGTTATTTCAGAGGAA
GAAATGATAATAATTTGGATAAAAGCATAATCAGAAATATACGCACAGTAAACATTTTCAGAATTGGCCTAGGTAAGGGGATGTTGT
GTGTGGGGATGGTAGATCAGAGTAGGCAGATCGAAACGGATTCCTCTTTTCCAGTTAGAATGTGGGGGACAGGAGGAGCCACCCCTA
GACATACCCCAATACATTCTTTTTCTGGAAAATTTATTGAATCATAAGGTGAAACAATCCTAGAGGCACACAGGCATCCCAGAGCTG
ACTCTCCCTCAGGAACCCTAAATTCAGAGTCATTAGAACAGTTCCACAGTATTTTAAATTTGGATCCACCCTGTCATTTGAATGAGG
CAACCAAAGGGGAACAAAAGTCATTCTTGGAGTTTACTTTTGGGGGGAACTCGTTTGCCACTTGACCAAAGACTGAATCACAATTAA
GATCTTAATAATGAAATGTTGTAAAGAAAGGCAGACATTGGTTCACACAGGCAAACATGAAATCAAGACTAAACTTACGCGTAGTTG
CAAAACTCCAAATTCTAATGTCAAAGAAAAAGTAGATTCTGGAAAGAGTGGATGTATGAAATTAAAGCAATAATTCAATAATAATTA
GACGGTTGCAAATCCTCAGATTAAAAATTTGAGAGGAGAAGGCTAGAAGGGATTAGTAAAAGCATGCCAGATGCGTTGTCCTATGTA
GAGGAGAATTTTAAAATAAAGCCCTATTACTCAGTTTTAGCAATCAGAGAAATATTTTTTTTAAAACCTAAAGGAACCACAGGCAAA
ATTTTATGAGTATAACAAGAATAACAGTAGCATCAGCCATATATTGTTTCACTTAATCCATACCATAGTTCTATGAGACTGTATCTG
CATTCTGTATATGAGGATGTTGAGACACAGAGAGGTTGAGCGATGTGCCTCAGGCCACAGAGCCAGCAGACTTGAGACTCCAACACC
AGCCTGAGAGCCTTTTCCCTACCCTGAAAACATATACCTCCCATGCCACATCAGAGCCCACATAGCTAAGACAGAAAAAATAAAGGA
AGCAGGAAGGAAGAATTTCAATACAAAGTATGAAAGAAGATCAGAGAAAAACAAACTTAAAATTTATAACAATCAACATAATTGTGT
TTACCTCCTTGTTAAAAGTAAATGTCTCTTAGATGAGATTTAAAAGCAAAATTTAATTGCATGCAATTTACAAAAGAAGTACCTAGA
ACAGAAGGACCCAAAAATAAATGAAGAGCAAAGATTTATTAGGTAAGTACAAATCAGATGGCAAACGAATAGAAATTCTCATTTTAA
ACCAAAATGAATTCAAAACAGAAATCATTAAATTGTCCAAAAGAACCATTTAATATGGATAAAAGTGCTATATACAGTAAAACTGTG
AATTTTATACAAAAAATGAGATTGATAATCTGAATTTTTAAATGTTTAAATTAATTGAGCACTTTGTAGCCTATAAACAAACATCTT
CTTTTTAAGCATCCAGGGAACACTTTAAAAAATTGATCAACTTCTTGGTCACACATTTCAAAACATCAGAAAAGTTGCTCGGGCTGT
ATTCTCTGACCACAAGCCTATAAAACTCCAATTAAAGGACAACTTTTTGGTGGGACTTTTAAAGCAATCTCCTCAATTACAACAGAG
AATTCAAAACTACCGTTAAAGGCTATTAGAAAATTACAACAATGATGACCCTGTACGTAAAAACTCAGGATGTGACCAAAGGTGTAC
TCAGATGAAAAACATCCTTAAAGTTTTTCAGGCACAATGGTGTGCACTGATAGCCCCAGCTACTTGGGAGGCTGAGGTGGGAGGATC
ACTTGAGCCTAGGAGTTCGAGCCAGTAGTGGACTATGATTGTGCCTGTGGATAGCCACTGCCTCAGCCTGGGCAACATAGGGAGACC
CCATTTTTAATAACAAAAACAGAAAAAAATGAACAGAGAAAATAACTGAATCCATTAGAAGAACAAAACAAACCTCAGGAGGGAAGA
TTAAGTTTATAACAAAGGCAAAGATAGGAATTAATGAATTTAAAAACCAGAAAAACATAATTTATAAATCCAAAAGATACCAACTTC
TAAAATATAAATTTAATTGGAGAAAAAGAAACACAAAATTAGAAATTTTAATGGAGAATAACTACTGATTCTTAGGATGTTTTAATA
AATAAGTGAGAATAGCTTAACTTTTTTTGAAAGAGAAGACCATTGAGGGGCTTTCTCTACTAGATATTAAAATGTATTATAAAACTG
AAAGTAATTAAGTCCCTCTGGCATTACTGAAAAATCAATAATGACAGATCAATGAAACCTAATGGAAAACACTGAAATAAACCCTAC
TATGTTAGAACCCAATATACGAGAAAGAAAACCAACTGGGAAGCGATAGATTATTCAATAAATGGTAATGGAGAAATTGGAAGCTTG
GATGAAAAATCAAATTGGAACCCTAGCTCTCACCAGCCACCAAAATAAATTCCAGGTGGATTAAAAAGTCAGATGTAAAAGAAGAAA
CCATGAGCAAGCTCCAGAAAACAAAGTGAATATTTAATCAGCCCTGAGAGGAGAAAGAACTGTTAAGCATAAAATCTCAGAAGAAAC
ATGAAGGAAAAAACTGGTGTGTTTGATTACATCATACACACAATTTTAGATTTCAGTCCCTCAAAAAATATGAACTTATTTAAAGAG
CAAATGTTATGCTAGAAAAAGTATTTGCAGCAAACATGACAAGAGGTAAATATCCCTATTATACAAAGCAGTCTTAGAAACTAATCA
GAAGAACTTATCTCCCCAGTCAAAAAAATAGGCTGAAGATAAGGCCAACTACAAAGGAAGAAATACAAAAAGCCAATAAAATAAAAC
ACACAACACATATTCAGTCCCTCTAATCAAAGAACTGCAAATTATAACTACAAGGAGAATTACTTTTTCCCCTCTGGAGTTGACAAA
ATTTTAAATAAATATAACTCAGTGCTGGAGAATCTTCTTATTGTTGGCGGTGGGCGGGCATGGGGTTGTCTTACGCTGAGGGCAAGA
AGGAAATTAGACCATCTTTCTGGAAAACAATTTAACAATTTGTCACAGGAGCCTTGAGTTTGTAACTTTGATCCAGGAATTCTCTTT
CTAGGAAGCTATTTTAATAGAATAATCAATTTACATCAAACCTCTTCTGCATGGTGCAGTTTATAATAGAAAAGAATGAGAAACCAA
GTAAACATCTAGGATTTCAATGGCTAAACAAGTATGGCACATCCATATGATGGGTAGTTAGCAAGAATTTTAAAAAATCAAATCAAC
GTGTTTGTTTTCGTTTTTGTTTGTTTGTTTGTTTGTTTGTTACTTCGTTGTTTGTTTTTGATGCGGAGTCTCATTCTGTCGCCCGGG
CTGGAGTGCAGTGGCGCCATCTCGGCTCACTGCAGCCACCACCTCCCGGGTTCAAGCAATTATCTTGCCTCAGCCTCCCGAGTAGCT
GAAATTACAGGCACCTGCCACAACGCCTGGCTAAGTTTTTGTATTTTTAGTAGAGACGGAGTTTCTCCATATTGGCCAGGCTGGTCT
TGAACTCCTGGCCTCAAGTGATCCACCTGCCTCGGCCTCTCAAAGTGCTGGGATTACAGGCGTGAGCCACCGCGCCCGGCCTAAAAA
TCAATCTTTTGAAAATTACAGTAAGGTAGGAAAATATTCATGATGTAATATTTGAAAAAGGAGCGTACAAAATAGCATTATGTAGGA
TATTACCGATTTATAGATAGGTGCAGAAATACCTGGAAAATACATAGAAACATTAAGTTGATTTCTTTGTGTTACATGTAATATTTA
TTTATTTATGAGATAGAGTCTCGCTCTGTCATCCGGGCTGTAGTGCAGTGGCGCAATCTTGGCTCACTGCAACCTCTGCCTCCTGGG
TTCAAACTATTCTCATGCCTCACCCTCCTGAGTAGCTGGGATTACAGGCGTGCGCCACCATGCCCGGCTAATTTTTGTATTTTTAGT
AGAGATGGGTTTTCGCCATGTTGGCCTGGCTGGTGGTCTGCAGCTCCTGGTCTCAGGTGATCCGCCCGCCTCAGCTTCCCAAGGTGT
ACATGTGATTTTCATACATGTAAACATTAAGTTGATTTCTTATGACGTTACATGTGATTTTTTATTACTTTTTATTGTATTTTCAAC
AATGAGAATGAATATACATATATAAATTTTTAATGGAAAGAGGTAGCATTTGTTTTTACACACTTGACGAAGCCCCTCATCTTCCAT
AGGAACTCATGTTTTACATCAACGCGGGGTGGAAAGTAAAGCTGAATGGCTCAGTTTTTACACATAATTTTCTCTAACAAGCGCAAT
GCAAGTTCCGTCTTGAAGGGCTCTCTTCCGACCCGTCGGCCAGTGGCCTTGGTGGACAGGGCCGCGGGGCGCCTTTGCGGGGCCGGA
GTGCTTCTTCGCGGGCGCGCGCCTCCCTTGGAGCCCCTCTGCGGCCGGCCGCTTATGGGAGCGCCCGAGCCGGCGTGCGGGCAGGTG
GAGCTCCACAGGGGACGTGTGATCGAGGCAGGCTCCACTGGCTCTTCAGTAGCCTCCCGGGCGTCCCCGCGGTGTTGCGGGGTCCGG
TGGGGAGAGGCCGCGAGTCCGCGGGTGGTTCCCGCGCGGCCAGGCGGTCGTGGAGAGGCCGGGGCCCGGGAGAGGCCGAGGCTCGCT
CCGCCAGGGGGCGCAGGGAGGGCCAGGTGGGCGGTGTCCCCACCTGCAGGGACTCGCTGTGGAGGAGCAGAGCGTTCAGGTTTCCTC
TCGCTGTGGCGACAGCCGGAGCGGCTTCAGGGCTGGGCCGGCGGCTCCAGGGCTGCTGCTAGCTCCTGGCTCCTGTCTCGAGTGGGA
TCCCATTCTGATTTCACCTGCGTGATTCACAGTCTCCAGTCCCACTCCGCGGCCGCTTTCTCGTTCTGTGATCTCGTATTCAGCCGT
TCTTTCCTCTTCCTCATTGGCAGTATTTTTGTGCCCTTTGACACTTAAACTGCTCATTCGTGGCTAGTTTTTTTCGTACTTTTATAT
TAAATAATTATACATGACTTAGCTATTATAAGTAATGGCTGATATCTCTTAGAAAGGAGAATCAAGAAATAGGGAAATTATTAGCCA
GCGTGGTGGCGCGCACCTGTAATCCCAGCTACTCGGGAGACTGAGGCAGGAGAATCGCTTGAACCTGAGAGGCGGAGGTTGCAGTGA
GCCAAGATCGCACCATTGCACTCCAGCTTGGGAGACAAGAGTGAGACTCCGTCTCAAAAAGAAAGAAAGAAGGAAAAGAAAGAGAGG
AGAAAGAAAGTGGGAGAGAGAGAGAAAATAAAATAAAAAAGAAACTGGTAAAGCAAGGAAAATTTCCATAAAATTTGCATTCTGAAG
CACTAAGTTTGGGACTGTGGGTGCAAAGTGAAATCCATATTGCATTGAGAAACCATTCCATTTTTTCAGTCAAGTCAACCCCCAAAT
TATTCCAAATTATTTGCTGCATTACACCAAACCTGGGTTTCCCTTCACTCTCTTCTGCTGGGAAGTCACCAGTGTCTTTTGTCTCCA
GGATTTTTGCCTTAGGGAGACCCAGTTAAATGCCAGGGTTCCTTCTTTAACTGGGGAGGACTATCTGACCTTTGTCACTGGAATGAA
TTCCTTCCCAAGTCTGCCAAAGATGGTACACAGTGACAGTTAGTGCCATGCTGGGTGTGTAGGAGAGAAGTGACTTCCTTTCCTACA
GCGGATGCCTAAGGGCATGGGGCTTCATTCTCACCCGCTCCCCCACCCACCTCCTTGGAGGGGCTGAGGTAAGGAAAAGGAAGGGGT
TCCTGTAAGTGACGTCAGCCACATCTATTTGTGGGCTGACATTAGTCAAGCAGGAAATCCATATTCTGTCAGCTTTTCCATCCTTCA
ACTTTGGGGTTGTTTTGTTTGCCCCTCGTGATCCCATTGTGCCCTACATTTCCGGTGCAGACCCCTCCCTCAAAGGACGGGCACCTC
AGCTCCACAGGGCCCCTTCTCCATGAGTTTGGTGTCCTAGGCTGGCTCTCAGGAGCGCAGCTCTTCACCCCCCTGTGTTTCCCCTGC
CCTCCACTGCCAGCTTGGGGCCCCCGCGCCAGGCTGGGGTAAAGCCCTGCATTCCTGGCTCACATAGCGCAGCTGCTAGAAGGGTTT
TTAAAAATGTGATTTTTTTTGGCCGGGCATGGTGGCTCACACCTGTAATCCCAGCACTTTGGGAGGCCAAGGTGGGCAGATCACCTG
AGGTCAGGAGTTCCATACCACCCTGGCCAACATGGCGAAACGCTGTCTCTATTAAAAATACAAAAATTAGCCCAGTGTGGTGGTGAG
CGCCTGTAATCCCAGCTACTCACGAGGCCGAGGCAAGAGAATCGCTTGAACTCAGGAGGTGGAGGATGCAGTGAGCCGAGATCACAC
CATTGCACTCCAGCCTGGGCAACAGAGCAAGACTCCATCTCAAAAACAAAAACAAAAACAAAAACAAAAACAAAACGATGTTCTCCT
TCCATTTAATACAGACTCCTCTGTTCCCTGAGACTTGGCCCCAGTCCCCCAGAGGCTGAGCTGAGAGGGAATATGGGTGAGACATGC
CATATTCCTTGAGTACCCCACCTCGTTCCATTCGGTCTGGATGCCTGCTTGCCCCCCACTGCACAGAGAGTTTAAGGGCAGCCAAAG
TCCCCTGGCTGCCTTTGAGTAGTGGCCCTGGAAGCCTGCTGGCCCTAACACACCTGGTCATTTTGTTTTCCCAAAAGGCGCCTGGGA
GCCGCCTGGTAACACAGCCCCTGCGGGCCCTCTCAGACAGGGCCAGACATGCTGCAACGGCCCCAGATGGGCTCTGACACTCCCACA
ATAGAGGGGGCACAGAACTTAGTAAAACTCCTTTTAAATATCTTAAACATGTTTCAGCTGAGCCAAGGGGAAAACACCTAGTACACA
GTGGCAATCGCACCAGCACAGGATCCATCCTGTGATCAGGACTGCCTGAACTCAGGGTGGAAAAACTCGGTTTGACCCCTGGTTCTA
CCATTAACCTTGCACTTGTACTTAACCTGACCAGAGTCATGCAGCCTCTTTTGAACACCCTCTGGGACACTACTTTAAAAGACTATC
CAATCCATTTGCAGACAGCTCTGACTTAGAAATCTCTTCCTATTATTGGTTAATATTGGTTAAAACGTGTAGTTTCTGTCTTGGGTC
CTAACTCAGTTCTCTGGGTCCGCACGAAAGAAGTTCAAGTACCATAGGCTGGCGGTTAAGCCATTGACCTCTGGGAAGATGTGAGCG
TGGAGCCTTGCTTATGCTCTTACTAGCAGGTGACGTTGGGCCAGGAAAGGGGATAATTCACAAAAAGCCCTTAGCACAGTGTAGAAC
ATTTAGCACACGGTGAGTACCCAGTGAAGCAGCCAGGTATTTCTTTTTCTACTGAAGGTCCCTTGAGTATCCAAAGTCAGGTTGAAT
CTTCTCTGCTCCAACCCAGTGGTCTCCAGTCCCCTCAACCATTGTCACAGGACTTCTGGTCAGACTCCACCCTCTCGCTCACTCTCC
TCCCCTGGTCATCAGTGTCCTCATGAAGCGAAGGCCCCAGAACGAACATAGTGCCCCAAGTGTTAGGCATCCTCCCATTTCCTCGGC
ATTATTTTCCTTTTAATGCAGCCTGAGAGTGGCCCATATTCTTAGGGCAAAGGGCTCCCAGGCATAATGGCCTGGGAATTAGGGAGC
GAGACCCAGGGCCCATCTCTGTTTAGATTGGGCACTCTTGACAGTTCCATTCTATAGCCCCATGCTCTATTGTTAACTTTTCTTCAA
TACAATTGTAAGGGCCTTGTTTAGCCTGAATCCTGCAGGGAGCAACTTGCACCCTTGTTCCACATGGCCACTACCAGATAAGAGCCA
CAGCTCAGTAAGGCCAGAGGAGTGGTTCAGCCACGTGGGAACCAGCCAGGACTCAAAGAGACAAGAGGCTCCTGTCCTGTTTCCAGC
TTTAGCAAGGGAGTTGAGGGCTGGCCTGAAGTCCATTTCTCTCTGCCTTAGTTTTCCTGCTGATAAAAGCAATAGTTAGTTTCTCAG
GCTCCCGCAGATTTCTCAACAGATTGTTTGAGCACTGAAGTTGAGATCTCTCCTGCTCTCTGGACACATCTCATACTGATTCCCCCC
ATACCCTATTTCCTACACAGGTCTGTCCTTTACCTGGACATTCTCCCTCCTCTGCCGTTTCCCTTCATCATGCTTGCTCCCGGAGAA
TGCAGGCACTCTCACAGGAATGCATCCATGCATCCCAAGTGGAGCATTGAGCCACGCCTCTCTCTGAGCTGCAGTGCTCTCATGAGA
GACTTGCTAAATAATAATACTGTTATGTATAGGTATGGAGCTCTGCTATTCACCAGGCACTGTGCTAGGAATTAAATATATCTTAAA
GCATAAAACACACATGATTTTTTCCCTCATGGGCTTACAGTCTACCAGGGAAGACAGGTAACAAGTAAGGAAACAAAAAGTAGATAA
TTTCAAATCTATGACATATACTGTGAAAGTGGAAAAAAGAAGTTTGCAATGATTCTTTTTTAAAAAAAAATTTATGGCATTGGATTG
GGATCCTACTCATCTGGGGCCTGCTTGGAGGTGTCTCTGGGGAGGTGATGCTTCAGCTGAGGTCTGAAGGATGAGAGGAAATGAGCC
CTTCACAGACCAAGAGGGTGAGCCTTCTGAGAAGAATGAGAAGACTGCACCAAGGCCCTGAGCTGGCAAGGAGCCGCGGGGAAGGAG
TCACTGGAAGGAGAGTAAACAGATGAAGGGGGGTGTGGCAGGGACTGAGCCGCAGAAGGGGGCAGGAGGCTGAAAGTGAAGGGCCCT
GAGTGCCATGTTGTGGGGTTGAACCTAAGTGCAGTGGAAAGCCTTTAAAGAGTTTTATCTTTTTATTTACTTGTTGTTTCTTCTTCT
TCCTCCTCCTCTTCTTCTTTTTCTTCTTCTTCCTCTTCCTCTTCCTCCTCCTCCTCCTTTTCATCGTCGTCGTCTTCTTCTTCTTTG
CCTTTGCCTTCTTGTTCTTCTTGCTCTTCCTCCTCCTCTACCTCCCCAACCCCTCCTCCTCCCCTCCCCATCCTCCACCTCTTCTTC
TCTTCTTCTTCTCCTCCTTCTTCTTCCTCCTCTTCCTCCTCCTCATCTTCCTCTTCCTTCTTCTTCCTTCTTCCTTCTTTCCCCCTC
CTCCTCCTCCTTCTTCTTCCTCTTCCTCTTCCTCCTCCTCCTCCTTTTCATCATCGTCTTCTTCTTCTTTGCCTTTGGCTTCTTGTT
CTTCTTGCTCTTCCTCCTCCTCCACCTCCCCAACCCCTCCTCCTCCTCCCCTCCCCATCCTCCACCTCTTCTTCTTCTCTTCTTCTT
CTCCTCCTTCTTCTTCCTCCTCTTCCNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNCCCTTCTTTCTTTCTTTCTCTCTCTCTCTTTCTTTCTTTCTTCCTTCCTTCCTTTCCT
TTCTTCTTCTTTCTTTCTTTCTTTCTTTCTTCCTTTCTTTCTCCCTTTCTTCCTTCCTTTCTTCCTTTCTTTCTTGTCTCACTTTGT
CACCCAGACTGGAATGCAATAGCATGATGACAGCTCACTGCAGGCTTTAACTCCTGGGCTCAAGCAATCTTCCCACTTCAGCCCTCT
GAGTAGCTGGGACTACAGGCATAAGCCACCATGCCTGGCTAAATACAAAAATTTTTTGGCGACAGAGTCTCTCTATGTTGCCCAGGC
TGGTCTTAAACTCCCAGCCTCAAGCAATACTCCCACTTCAGGTTCCCAAGTATTAGGATTATAGGTGTGAGCCACCATGCCTGGTGC
TTTTGAAGGGTTTTAAATAGGAGAGTAATACAATATGGTTGCTTCTAAAAATATTACTTTGGCATCTTTGTGAGTAAATTGGAGGTG
GCAAGAAAACTACTAGGAGGACTAAAAAGGCTACTGCAGCAGGCCAGGCAACAGACAAAAGTGACTTAGAAGGGCGGTGGTGGCGGT
GTAGAGTTCTTGAATACATCCCTGCACCATGCCAAGTTTTATATACATCTTCTCCTGTACTGATGTCCCACGCATCAAATCTGTGAT
CAAACTCATCATGTTGCCCCTAAATTCTCTCTCTGCTTCTCTGTTTGTGTCTTCTTTTTCTCAGGAACTTTACCCGTTAGTATTCGT
TTTAGGCTGCAAGTGATAGGAAACCCAAAACAACAATAGCTTAAACAAAACAGAAGTTCTGTCTGACGCCAAAGAAGTCCGGAAAGG
TGTCTATAGCAGGTATGCTGCTCTATGGCATCATCAGGGACGCAGGCTCCTTCTCCCTTGTTGCCCTGCCATCGTTGGTACGTGGCT
TCTTCCTCATACTTCAAGAAAGCTGCTGGAGCTCCAGCCATCCCACCTACCTTTTAGCCATCAGGAAAGAGAAATGGACAAAATAAG
GTTGTTTCTTTCTTGTTAAGGACATTTCCTCGAAGTAACACATGTCACTTTCATTTACATCCGTTGGTCATTCACATGGCGGGAAAA
TGTAGTCTTTATTATTGGTAATCATTGCTCCTTCCTGGGCGCTCTTACTAATGAAGAAGAGGAGACGTGCTGTTGGGCTGCAACCAT
CTCTCAGGCTCAAATCGTTCCCCATCCTTATGCCACTCCCAGCCCCATTTCCAGCCCTCTATTTAGGCACCAAGCTCTATGGTTCTT
ATTCTGCGGTGTCGCTTGAAGTCATCTCTTCCTGCTACCTCCCTGACACAGATCCCTGTCATCTTACACCAGTTTACAGAAGTAGCT
TTTCTGGCTTCCAGTTACCGTTCCTACTATTACCCCTCAAAAACCTTCAATTGCTCCCTCTAGAAGATTAAGTCCATATTTTTAAAT
TTAGTGACCAAGGCCCTCCATCATGCAATTCCAGTCAACCCATGATAAAACCGGTTTGGTTCAATTTTAGTAACATCAGCAACAATG
GTAATAATGATACTTTGCATTCCTACATGGAAGGTTTTATTGCTCACAGAAGGGACAGCACATGGCCCCTGGAGTCAGAAACGTGCG
CTCATAGCCTGGCTTGACAACGGACCAGCTGTGCGATCCTGGGCAAGTCACCTGCCCTCTATTAAATAAGCACCTGCGACTGGCTGG
TTCCTGATAAACCCTGAGCCCTTTCCTTTCCTGGGCCTCTTTCTTGGGATCTGTAGATAGGAAGTAAGCCTGGTCTACCTGCTTCCC
AGGGGATTAGGAAGTTCAAACAAGAGGAGACCTGAACAGCTGGGGGAAACATAAAACACTGGACATCCTCTTCTGTTTTTCACCACG
ACCCTATGAAGTGAGCAGGCAGGACGGGGAACCCACGGTCCCTATTTCGTGGATGAGGATGTGGTGGCTCAAGGAGGCTCATGATCT
GCCCTGGCCCTGACTTGTAGGGCAGTGGAGGAGGCCTGGGCCCTTGGGACCCCGATCGCCTGTGGCAGGGCTTTTGGGGTATGTGTT
CCACTGCGGAGGTGTGTTACAGAGTGAGTCTGTGTTGAGACAAAAGGAGAAAGAACATAAGACGCAAAGTTTTTCTTTCAGCTTGGA
CTGTGCAGTCATTTTCTAATTTCTTTGAGGTCATGCTGTTTGGTACCTAGGAAAGGAATTGTGATTGGTGTTCTAGAAAAACAGTGA
TTTTTAGGGGGAAGCCACTGCCTATTAGTAAGTACAGGCAGAAGCGGCTCATTGTCCTTGAATGCCATGATGTCTGTTTTCGAGGGG
TGGGGTGGGGTCTGGTGACTGGCTAGACTTTTTTGGAGAAAATGTATAACAAACTTAATAAAGTAAACATCTATGTTGTCAAACTTA
CTGAGTTAAGTAGATGTGAATATAGTTAACTGAGTAGTAGCTTTAGAATTATTTTGTCAAGTTTAACATGTGAAATTGTTCCCACTT
AGTTACCAGTATTCTTAAATTGTCTTGTTTATAATTTAAACTGTGCATTTTATTTAGAGAGTAAATGTGTCTGGAGGCTTTTCATTA
TTTCAGAAATTAGAGGATAGATTCCTGAGGTGCCCGGCCTGTCCCTGAGGCCACGTGATGCAGAACTGGAGGCAGGTGAGTCTGAGC
CGGGTGCTCGGAGCCTAGTCAAGGGTGACCACAGGCCGAGGGAGGAGAAACTTCTGACATGCAGTGTCCTCATAGGAATGCTTTGTG
GTGTTATCTCCATTTCCCTGATGGAAAAACTGAGGCCAAGAGCAGTGGGGTAACTCCCCCACCATAGTGTGCAGTTGCTTAATTGCC
TCGCCCCCAGCAGGGGGCATGTCTGTGTGATAATGTCTGCTACAGTGCCCAGCCTCTGTAGGAGGCTAGGAATTGTTTTTTAATAAA
TAAGGAGCACTATCTTCGTTCATGGCCATTCATTCTACACAGGTTCTCCTCTGTGGCCCAGCACTCTGTGAAGTAAGCAGGGCAGGA
GCAATTTTCCCATTTTAGAAATTAGGAAATGGAGGCTTTGGGAGGTAAGCGAGCCAGGGTCTCACTGCTGGCCCGGGGCTGCTGCCC
GCAGAGTCCTCCACCTGGAGCACTCCCATTCTCCGAGGTTCCCTTGCCAGCTGGGCATGGCTGTCCAGACTGAACATCACTCCTTCC
TTCCTTCATCTGCTCAGTGCTTGAAGATCCCAGAGCCCAAGGTCACTTCAGCTAACAAGCTGATGTCCCTATATTTGGGTGAGCTGA
TGGTGCTAATGGCAGTGTAGATCTTCGTTTTAGAGGATAATTATGTTTTCTGAAAGTCTGTGGAGAATTGTGCCATTGCTTGAGGGA
TATCTAATAAAAATCACCCACTGGATAAAAGCAGACCAGAGTTCACCTAAGGAATCATCATCGTCATCATTGTCATCATCTTCCTTA
TCAACAGCCTCATCTCTACTCATGACAGATTTGGCCAAAAAGAAAAAAAATAAAGGAGGCCTTTCGAGGACTTAACACCCGGAGGAC
TTTCCTCCATGCCTCCTCTCTCTGTCTGTCTTCCTAATTTCAGTGAGTCAGTTTTTCCTCCCCATCTGTTTGCTTTCTGGCACTTTA
CACAACTGTCTGGGCTCTTTCATTTTGCCTTCTGTGGTTGCTCAACTCCATCATTTCCCTCTCGATGCTTCTCCTTTGTTCCCTGGC
AGGCTCCTCTCATCTGCCAGTTTCCAGTCCTATGAAGAGTCAGAGGTGGGTTTTAGTTTTCAGGCAGCATTTTTGTTGCCTGGCAAC
GTGTAACAAGCATCTTCTGTGGTGGCTGCTTCCCTTAAAACAGAGCCAGGCTGGGGATGCAGTGCACGAAGGAGTCTCTGGGAGCTT
CTCTGGGGTCATGGGGAGGAGTCAGGGGAGGGGGACAGACAGACAGGTTGGCTTCCATGGTGACTCTGAGTGTTGCCATTCTTACTC
TTTTATAATAGCCTTGGCACTGGCACGCCCTCTCCTGCCCCATTTCAGGCACCCCAGGTGTCCAGTGGTGAAAAGCCTTGGGTTAGG
AGCCATCAGAAGTTCTGTGTAACTTGAGAGGCACCTGCTGCAGCTCCCTGAGGCATGGCCAGCCTGGATGAAGGCTGCTAGGTGGTT
TCCACTGGTGCTGACTCTTGCTTCATCTTGTCTGCACGGGATGCCACAGGCCTCCTGTCTGGAAAGACACCTGGCTTTTGCCTCTGC
AGTCAGCCCAGCTGCTAGTTGTCCCGAGAATGGGAGTGGGAGAGAGAAGGGGGACAGAATGTGATCCAAAAAAACACCTGACTTAGA
GCTGGAGGGGGCTTTAGAGCTTCTTTAGCCAAATCCTCATATTGTACACACGAGGAAACCAAAGCCCAAAGAAGGGAAGCAACTTGT
CCAGACTCACACCGCGAGTTGGGCGGGTCTGAGCTCCCAGCCAAGGGTCTCTCTCCTGGGACTGGACTCTGCCGAGCTCACACATCT
GATGCCCTGTGGCTAGAAAGAGGCAGCATGTGGTGAGAAGTGCACCAGATGCCCAGTTCTCCGCTTTCTGGCCTCCATTTGCTTATC
TAAAAAGAATGAGGTGAACTGGGCTAGAGGGAGCCCCAAGACTCAGTGACTCTGATGGTAAAGCTGACACAGCAGGCAGGCCCCTGC
TGCCCAGGCCTTCAGCCACGTGTCCAGGGCTGCTCTCACCGCCCCAGGAAAGCTCAGAAGCTGAGGGGGTCCTGGGCCTCTGAGCTG
ATGCTCCCCAGGAGAATCCCACTGTATGCAGAGACCCCGAGCACAGCCACGACACTTGTCACCATGAGGCTGATTTAGGGCAAGCCT
CCTGGGCATCTGTGTCTTCAAAGATGGTCTCTTAAGACCCTTATCAAGGATCAGGGCTGCAGCTGCCCACTCCCCTACCTAACCAGC
TCTCCATCTAGTGTGGGAAACGGGTAAGAAAACATTCTCTGAGAATTACAGCTCCTTAGAGCTGGAAGGGGCCCTAATGCACATGGA
CCCAGCCCCCCACCCTTCCCAGATGAAGAGAGGGAGGCCCAGAGCTGTGAGAGTTGATGCGAAGGGGGTTTTGCTCTATTCCCTTCA
GACCCCACAGGTGAGAAGGCATCTCCAGATGGGTCATCTCTTTATCCTGCTCCTTCCTTTCCTTAAGTAAGAATCTCTCAGTTCAAG
AGGCAGAGAAAAGGATTGCTGAAGGCAATCTGTCTCTTCATATCTCTTTCATTCTGCTCCTGGGGCCGAGGAAATGGGTGAGGAGGG
GTGCAGGAGGCCCTTGGCCTGAGCACCAGGCCTGGAGCCTTCCCTTCCAGCCGCTGCTCTCCAGAGCTTGGGGTGCACCAGCTTCGA
GGCTTTAGAAGCTTCTCCAGCAAGAAAGGCCCTTCTGAGGGAAAAGTGTGTGCGACTGGCTGTCTCCATTCCTTTCTCCATATAGTG
TTGACCTTCGGAATTAGCTTTATTCCTGACGTGTACCAGAATTTTCAGTTAATCATGTAAACCAAGGCTCAGGTGACTTGGCTGTGG
GCAGTTGGGGCCGGACCAGCCCCTGAGAAACTACTCCCATCCCAGACTTCGTGACGCTTCACTTGGCCAGAGCCCACTCCCAGAAAG
CAGGGAAATGTGCCTTTCTGCGTAGCCCAGGCCTATGGAAAGAGGCGAGAGGTCAGGGACAGCTGCACTCCCTGGGCAGGCTGCTTG
TGGTCCGGGAGCATCCCGTAGTCTCCCCAAGCCCCACGGCCGTGGGGACCCCGGGTCAGGGCATTATTGTGCATATCCCTGTCCTCA
GGCCCCTCATGCTCAGGCCAGACCCTCACCCAGGTGCCCTTGCCCTCAGACTCCTCCCCTTCCAGCCATTGTCTCTGTCTCCATCCC
TGTCCCTCTCTTTCTCTATCTTCCTCTCTCTCTGATTCCCTGGGGGCTACTTTGCAACCTCTGCTGAGTCAGAATTCTTCCCACTCG
CCCTGACGTCCCAGGCTCCTCCTTGGCACCCTGTCTTCTCTGTGGCAGGGAAAGACGCCCAACCAGAGCTCCCACGTCCTGCCCTGG
GAACCCCACTGCTCTGGGAAACAAAAACAAGAAAATTCCTTCCCAGGGTACTTGTTCTGTGCTGTTCCTCTTTGTTCTTGGTCACAG
GGTGATTCCCACGTCCTTCCCACACTAACCCTGCCTAGGTGAGGCTGCAGCTTTGTGTGCCTTCCACAACATTTGCTTGCCTGGGGA
GCTGCCTGCCTTTAATTATCTTTGGAAAGCATAACTGAGAAGAGCAAGACACCCGTCTTTTCTTTAGGGCTCTTGCCAGGAGCACTG
CCTATGCAGCACTTCCCTCCAGGGGGTGCCATTCACATTGAAGTCTTCGTGACTGGCACTCCTTGGAGTTGCACAGTGCGCAACCTA
TGCTGCTGAACAGGGCAGCCCTGGCTCTGTCTTACTCTCCTGGGAAGCATGGCTCATTATAACACAGATCCAGGATGAGATACCTGA
GAGAGAGAGAATCCCCACCAGTCTCTCCACTCATCTCACCTCCCTTTTTAGGTCTCCACCTCCTCCAAGGTGTAATATGGTCAAAGA
CAGGGCTCCTCCAAGGTGCAATATGGTCAAAGACAGGGCTGTGGATGCAGGCAAGTCCAAGCACACACTCGGGCCCTGCCACTCAAT
TGATGTTTTATTTTGTGCIAGTTTCCTAATCTAGTAATTAATCAAGTGATCCTCCCACCTCAGCCTCCAGAATAGCTGGGACTATAG
ACACGTACCACCATGCCCTGCTAATTTTATTCATTTTTTGTAGAGACAGCATCTCACTATGTTGCCCAGTCTGGTCTTGAAATCCTG
GGCTCAAGGGATCCTTCTGTCTCAGCCTCCCAAAGTGCTTGGATTACAGGCATGAGCCACTGCACCTGGTCTAATTTTCCAATTCAG
TAAAATAGGAAGTGACAGTTATGAGTCTTGTTTTTTGAGGATTAAATTCAGTAATGCGCATAAAGCTTTGGCCCACATGGCACGTGA
TTGGCACTTGAGAAATGATAGTGATTATTGTTATCTCTTCAAATAAGATTAGCATCCAGTCTTACCTACCACACAGAGATAGCCCCA
GAAATAAAATGAAATGAGAAGTAGATCATCTCTAAGATCTTTGAGGAAGTATTTTGCAAGAATCCCAACTACTGTTGTTCTTCTCTC
TGTATATAAAAAACGATGAACTGTGAAGCCCCTTTGCTTCCAAGTCACGCTCTGTGACTGATGGCAGATGTCGGGAAAGGCTCAACC
AGGAGAGAGAAAGCTTGCAGGCGCGGGCGGCAGACTGAGGGCCCCGGGCGGGTGGAGCTCAATGAAAACCACAGACAGACAGCAGCT
GTGAGAGCAAGCAGAGACCCAGAATGGCTTTAAACAGAAAGCAGAGGCCCACAGCACGGAGTGAGATGAGACCAGGTTTAAACGTCA
CTTTCCCCTAAGTCCTTGTTCATTCACAGCTTCCTTCAGTAGCTCTGACCAGCTTTCTCAGCACCTGCCGACCGACAAACCAACCAA
CCAATGGAGGCTGGTGCTGGGGGTGGAGATGGCCAAGAGCCGGTGGTGAGAAGTGAGGGAGAATGGGGAAGAAGGGGCTCCACAGAG
GGCACTTGATCCGGGACGGCTTTCCGGAGGAGGTGATTTCCTAGGCCTAAGGGGCCTCCGGGCAGAGGGGCCAGCATGAGGGAAGCT
CGGAGGTGGGGGAGCATGGATTGGAGGAGAGAATAAGCAGTTGCTTGCTGGGGTGTCATAAGATGCGGCAGTGCACTCAACGCTACA
GGAGCAGGCACGGGCCTTTTTCACCAGCCTAAGGATTTTGGAATGACTCTTCTATCAGCCAACAGTTTCCAACCTTATTTATGTACT
TGTTTCTTTGATTCCAGCGATGCAGGAGAGATGATATTCAGACAGCCGACTTCTGCTGTGGGTGTGCCCCAGGTTATAAATCATTTC
CATCAGGCTAGCTGGAGTCCCGGCCCTTCCTTTCCACTGAAGAAAGCAAATCAGAGTACCAGATAGCGAGAGTGATATTATCACAGG
ATAGCCTTGAAACTGCCATAACAAAAAAAATAAATCGTTCACAAATGTCAATCCTTCAACTACTATTAGCCCCAGAGAGACTGTTTG
TCTACCCCAGTGTGTCGCTGTGCAGAGGCTGAGAACGTCCCCTCTCCTCTTCCCACACCTAGGAGAGGGGGAGCCACTGGTAGATTG
GGAGCAGGGGAGGGTCTGGAGTTTCAGAAAGATTTCCCTGGCTGCAGTGTGGAAGGGGGATTTGAGGGGAAGACGGGACACCAGCTG
GGGAGGCTGCTGTAGTAACAGAATGCTGCCCCCACCACCCCTGGGTACAGTGGTGGCTGGGATGAAGAGCAAGGGCAGTTTTGAGAG
AGCTAGAGGAGCCAACCTCCACCGGACTGACTCACTGGATGTGGAGTGATGGGGAGGAAGCTCAGGAACAGTGTCTGGTGTCCTAGT
GTGGCTGGCTGGATAGCTGGTGGGACTGCCCACGAATAGGAGGTGCAGGAGGAAGAGGGCCGGGGTTGCAGGGGACCTGGGCAGCAG
GGAGAGGGCAGGTAATGAGCAGAGTTGGGGATCGGTTGAGCTTAGTTTACCTGCCACCCCTCCAGAGATGTCCCACGTTGGGGGGAC
CCATGCACCTACTCCAGATGTTACCCTCCCCACTTTACAGCACAGGATTCTGCCTGGTGACATTTGATGCCATCTGTATCTGGCCAC
CCAGTGGGTGCAAAATGCAAGTTTGCTGATTAAGCAAAATCAACCTGAAGCAAACAAAGACTGATGTCCCCAAGGGATAGCCACCTC
CTTGGAGGCAGAAACCCTTTCCCTCTAGGGCAGAGCTTCCTCTGTGGGTCCTCACAGGGGAGGGTGGGAGAGAGACTGAACAAGTGA
AAGGGAAAACAAGAGAGGAGAGATGAGAGGGAGGTGGAGACAGTGCTTCAAAGGAGAAAAAAAGAAAAAGAAGCATGAAAGACAAGA
AGAAAGAACAGAGAGGGGCAAAGGCAGGAAAAATAGAGGAGAAGGAGGAAGAAGATGGAGGGGTTTGGGGCAAGAGTAACAGTGCAG
GCCAGCTGTGCCTGTGAGTTCACGGGGTTCTGCTTATTAAACCCCAGCACTGGCAACTTTGACAAGATGCTCAGAGTTCTTTCCCCG
CCCTGCTTCTCCCTCCAACTCACCTCAGCCTTCTCCTGAGCCTGCCACTCCACCCGCAGCACTGTCATCCCAACCCATACCTGCTCC
TCTGAGATAGCTGTGTGTCCCCTGCGCAGTGGCCCCGTGCCCCAACTTGGCGTCACTTGTCTCCATTGTAACGCGGCCCTTTTTTGC
CTTGGTGTTGCTTGGTGTCTGCCTTCTTCCTAGAACTCTAAGTTCCCAAGTAGGCACCAAGCCAGGCACACAGGAGGGGGCTCATTC
CTATTGCTGACCAAATGAACTCATTTACTAAGTTCCAAAATTTGTGTCAGGCACTATGCAGAGTTTTATATTTTTCACTAGTCCCGT
GCAAGGTTTTACTGCTTCATTTTACAGAAGTGAAGCCCAAGACACAGAGAGGTAAGCGCCTTCCCTAGGCCACACAGCTAACAGGTG
GCAGGGCTGGGATCCAAACCCCCGTCTGTTGGACTCTCTGCCCATGCCATCTCTGCCACGCCATCTCCAGATACCGTGGAAGACATG
CTTCCCATGGGAACAGTGCTTCTTCCAAGCATATTATTTTTCTCCATTTTCATGCATTTTATTAAACATTTCCAAATCAGGAAACAT
GGAAATATTTTTTTAATAAGAAGAAAGAAAACATTGCAAATTGAGAAACTGACTCAGAAGGATTTGCCAGATGAGCCATCTGGTTTT
TAATTTCAAAGAGGTTGACAGTGCTTCCCTCTAAGAAGAAAATGGACTGTGGAAGGAATGCCAGCTGGCTTCCCTCAAATGGTGCAT
CCATTTGCAAGGTGGAGAAGTTGAGCAAGACCAAGAGACCCAAGAGAACAGAAACACACTCCAAGAAAAGCTTGGCCAGTGCTGGTG
TTGAAGCTTCCCCGCTGAGGCCTGCAGAGGTTGCATCCAGCTGTGTGACACCAGCCAGCTGTCCCACCTCTTCCAGAGTTTCTCAGA
AGCTTCTACAGGAAACCACTAATGGAGACAGAGATGCCACCACCTCGGTGAGAAAAAGCCACACCAACCAAACTCTCGTTTTCACAG
AGCAAGAACAAGCTGTGGTTGTTTGGCGGCAAGTCGCATTTGTTTGTTGGAGCCATTGTGCTCTGGTGGTGCTGGTTTTCTCTCCTT
TTAAAAATAACTGGAGGCCTTGCACCATGCTGTTAGTTCTTAGGGCTGGAAATAAAAGACTAGATTTAGAGTTGCCTTGGAAAGATC
CAGGGCTGTTACCTTGAATTACCTGGTAGAGGAAAACGTCGTTATTTTCCTAAATTCAAAGCCAACTCTTGAAAAATAGGACACAGA
GGTGAAGTGGATGGAGGGGTGGCTTTATACATGCATGTGGTGTCCTGGGCACGTGGTTTGATAGGCTTGTGATCAGCCCTGAGTCTT
CAAAGCCATTTATTTTACATTTTAAGATGTTTATAAGGGAAATTGTTTTTGAGAAAAGAACGAACTATTTTTTTTTTTTTTTTGCCT
TAATCTGAATTCACCCACTGGCACAAGCAGTAATAGCCCTGAAGTCACCCTAGGCCATTTATTCCTCAGCTGCCTTTGAATTTCAAT
GGGATTCTTTCACACGCCTCAAGCATTTCTGAAATGCTCCTAAACTAAAGGAACTCATAATACCCTTAAGAAATGATGATTTCACCC
CATTTTCTCCATAAAGCTGGAATCACATGCCCTAGCATGCTGAAGGTACTTGATAATTAGAAGTTGATTATATTGGGGACCTGGACA
AAGGCAGAGCTGGGAATGGGAATAAAACCCAGGACTCCATGAAAGTCTCTTTCCTTTCATAATAGCTTCTAATTCAGGCTTCTAATA
GCTTCGAATGCCAGTCACATACAGAGCCTCCCGGCCATCACCTACTGGCAGTGGCTGAGCGGGTATTTTGCCAAGCTACAATGGTTC
CTGCCCAGGCACACGGGTGCTGCCTCCACCCCCCACCCTGTGCCCAGCTCACTACAATTGCCATCTTTAGCAGCCGAAAAAATACAG
GTTTCAGTCCCAGTCTTTTCGCTTAGTTGCTGTGAGAAGTTAGTTAGGTCAGTTACGCACTGGAGCTTTGCATTGATTCACTGTAAA
ATCAGGTAACAGCTCTTATCCTGCTGGTCTTGTCCCACTGGCTTGTGAGAAAGACGAAGTGGAGCCATTCATTCATTCATTCATTCA
TTGGACAAGTATTTATTGAGTACTAGGGATACAGCAGTAAAGAAAACAGAAAAAGTCCTTTCCTCCATGAACCTTATATTGTAATAG
GGGTTGTCAGTCAATAAATACAAAAATAGGCCACATATCTATTATGTCAGGTGGTGATAAGCGCTGTGGAGAAGAATGACTCGGGAG
AAGCTGGCCTGCTGGGTGTGGGGCAGTAGTGCACTTGGAGACAGGGTGGGCGGGTAACACCTTACTGAGAAGCTGACATTTGTGCAA
AGGCCTGAAATTATCTGGGATAAGAGCCTTCTAAGTAGAGATGTCCCGTGACTTATGATGGGATTACTTCACAATAAACCCACAGTA
AGTGGAAAATATCATAAGTTGAAAATGCACTGAACGTACCTAACCTACTGAGCATCATAGCTTAGCCCAGCCTACCTTAAACGTGCT
GGGAACACTTATGTCAGCCTGCCGCTGAGCAAAATCACCCGGCAGCACAGTGCCCTGTAGAGGACCTTGTAGAGTACTCATGATCAC
CTGGTTAACTGCGAGCTGCCCAGCATCAGGAGAGAGAATTGCATCACAGATCCCTAGGAAAAGATCAACATTCAGAATTTGAAGTAC
GCTTTCCACCGACTGTGTATTGCTTTTGCACCATTGTAAAGTGGCACCATTGTAAGTCAGGGCCATCTCTGAAAAGAACAGCAGGTC
AGGGGCTCTGAGGCAGGAGTAAAGCTGGCTGGGTGAAGGAGCTGCAGAAACCCCAGGTGGCAGGGCCAATGGGCGAAGGTGAGATGG
GCTCAGAGAGGCCCCCAGGTTGGGGGCCCCAGGGGACTTCGGCTTTTTTTCTGAGGGATACAGGAACTTTTGTCTTACTTTTTTTAG
AGGGCCTCTCTGACTACTGTAACAGGACTAGCCTGAGGCAAGCGTCGCCCAGGCAGAAGCAGGGAGGCCAGTGAGGCAACTACTGCA
GTGGTCCAGGTGAGAGGGCGGTGCTGATCCAGGTGAGAGGGTGGGGCTGGAGCAGGTAAGAGGATGGGACTGGAGCAGGTAAGAGAG
TGGGGCTGGTCCAGGTGAGAGGATGGGGCTGGTCCAGGTGAGAGGGTGGGGCTGGACAAGGTGAGAGGGTGGGGCTGGTCCAGGGTG
GCCACAGACCAGGGATGACAGGCGCTCCGATTCTGGAGATATTTTGAAAGCAGAGGTGACAGAATTTGATGGAGATAAGGGGTTTAA
GCTGCAAGTCACCAAATGAAGAGAAGGGGCTGCAGCCACCCAGAAGAACAGCGAAGATTAAAAGGAGAAGCTGGGAGGAGTTTCTGT
TTTCCTACACAGCAGCATCTGCTGCCTGTTCTTTCTCCACATCCGTGCAGCTGCCCTTGTCACTAGGACGAAGCCCCAGGTGAGAGG
CTCCTGCAGAGGCTGTGACATACCTCTATCTGTATCACTTGCTGAAGGCTGATGGAGCCCGTGCACATTCACTCCTCACCGCGGCCT
CGCGAGGCAGGCAGGACGGCATCATTACACCCTGCTTCCCCGAAGAAGCAGCAGGCCCTGAGTCCGTGGAGTGATCTGCTCAAGGTC
ACAGAGCCATTTGGCATGAAAGGCAGTCTCTGGGCTCCAGATCTTGTGTTCTTTCTGCTATGCCAAGGCTAACCATAGACACACGAG
ATGAAAATAGCACTTTTTTTTAGTGCACCTATGTGCCCGGCACTGCTCTAATCATTTTAGACATATCAGCTCCTTTAACCCTCACAG
TAACTCTCTGTGAGGTGGTACAAGTATTATCCTCTTTTACAGGTGGGAAGACTGAGGATAAGTTCATGCAGACATTAAGGTGCAAAG
CCCGGATTCAAACATAGGTGTTCTGGCCCTGGAACCTGTAGGTTCAGCTATACCCCAGCCAGCTTTGCGGCCTTTCTGCAAGCCAGT
GTGTGGGCAGTAGGTGGATGGTGCTGATGCCACGTGTGGGAGACACAGGCAAAGAGCTGGGGTTGGCTGGGTGGGTCAGGATGGGAT
TTGAAGTGGGTCCTGAAGGGCCAGTTAGAAGGCATCAGGGAGGACAGGGCAGGAGTGACAGGAGCAAGAGAGCCATGTGTGCAAGGC
AAGCTTGGAGCACAGCAACCCTGAAGCGGTGGTCAGAGCATGAAGCCAAGGAGGCACAGTCGAAGGCGGGAGGAAAGCAGGCCAGTG
AGCCGCTGCAGATGCCAGAGGAGGGCAGTGTTCCGAGGAGAGAGGCACCGGTGCGGTCAGAGGCCGCCAGGGGGCCAAGCCAGGTAC
GTGTTGTTGGCTTCCTCATTGGAGAAATTCGCAAGCACAGGGTTCACAGCTCAGTGGGCAGGTCTGACTGTGTGACTCTGAAGATTG
ACCGGAAGGCAAGGACGAAGCAGTCCATTTGGCTGCTTTGGAGTTCTGCAGAGGATCTGGCTCAATGCCTGCAAATTTCCAACAAAC
AAATCAGACCCAGGACAAATGGCTAGAGCCGTCATATCATGTAGATCCATGTCAAAAGAATCCTTTTTTTTTTTTTTTTACAAAACT
AGTTTTAAAAAGTATTCAAATTCTTTTGGAGAATAAGGAAGAAGAGGCAGTCATTTCACTTTGGCTTATTTTCTGTCACCCTAGTGC
TGTAACAGATCACAGCTAGTGTCATGAAGCTGAGTTCTTATTAATAGTGAGAACTGACTCCAAGCTCAAGGAATTTGGCAGCCACCT
TGACAAAGGTTTAATCTGCTGAAGAGAACAGCTGAAATCATTTTAACATGATTCACTGAATTACTGAAGCAAGCCTCACTTTTGTAG
AGGGACAGGAAAAGCAGCATGCAGGCCTTAGTCAGTCCACAAGTATTTTAGTGAGCCCCTATTATGTGCTAGACCCTCAGAAGGTAC
CAAGGAAAGGCTACAGAGAATACTGTAATATAACTTCAGGGTTAGACAAATAGACCAATGGAACAGAATAGAGAACCAAGAAATAGT
ACCACTTCCACATATAATTTATGAGACAGAGGGAGCATTGAAACCTAGTAGGAGAAAGGATGGTCTGCACATTAAGTGAAACTGAGA
TAATTGCTTACCCATGTGAAAAAAATGAAGTTGTATCCCTGCCTCACACCATATGCAAAATCAATTCCAGGCAAATTGAAGACTTAG
AAAGGCAACATTTTAAAATATGTAAAAATGTTACAATCTCAGGGAGGGAATGATTTCTGCAAAAACCAAATAACTGCTAATTATAAA
TTAAAGGATGATAAAGTCACCTACAGTAAAATTTAAAACTTCTATTCATCAAAAATACTGTAGAGAAAAAGGCAAGGCACACACTGG
AAGAAGATATTTGCAACACACAAACTGACAAAATTCTTATTATCTAGAATATATAAATAACTTTTAGAATCAATGAGAAAAAACAAA
AACAAGCCAATAGAAAAGTGGACAAAAGTCATGAATGACACGCCACAGAAGAGAAAGTATGAATGACAAAGAAACATATGAAAGGCT
ACTTAAGCTTTTATCATGGAAACGTAAAGTCAAGTCTTGATGAGATCCCAATTCACCCACTAGTGTGGCAGAGATTAAAGTCAGACA
ATGGCAAGTGTTAATGAGGATATGGAGAAACAGGAACTCTTATCCACCTCTGGTGGAAGCCTAATTGGCACTGGCATCTTAGAAAAC
AACTTGACATTTCCTAATGAAGTGAAATATGTACATATCCTACAAACCAGCAAACCCAGTCTTAGGTATGTGTCCTAGAGAAACTCT
TATGCAGTGTACCAGGAGCTATGATTAAGAATGTTTGGGCTGGGTGAGGTGGCTCATGCCTGTAATCCCAACATTTCAGGAGGTCAA
GGCAGGAGAATCTCTTGAGCCCAGGAGTTTGAGGCTACAGTAAGCTATGATTGTGCCACTGCAGTCCAGCCTGGGCAACAGAGTAAG
ACCCTGTCTCTAAAAATACAATAACAATAAGTCAATAAAAAATAATGTTTGTAGCAGTATTTCTCATTAGAGGAAAAACCCGTAAGC
TATCCCACTGTCCATCAACAGGTGAATGAATAAATAAATCATGCCATATTCATATGATGAAATACAATACAGGAATAAAAATAGACT
ATAGCTACATATGTCAATATAATATATATGTTGAACAGAAAAGAAGCAATTCATAGAAGAGTTCACTGTATTAGTTATCTGTTGCTA
CATAACAACTTGCTCCAAAATTTAGCAGCTAAAAACAGCAGACATTTACCATCTCAGGGTTGCTGTGGGTCAGGAATTCAGGTGCAG
CTTAAATGGGTACCTCTGCCTCAAGTTCTCTCATGAGATTGTCAGGCTGTCAGCCAGGGCTGTGGTCTCACCTCAAGGCTTGACTTA
AAGAACACCTGCTTCTAAGCTACTCATGTGGTGGTTGGCAGGCTGTGTCCCTTGCCATGTGGCACTCCACGGGCTTGCCTCCCGACA
TGGCCTGTGGATTCTCCCAGGGAGAGTGATCCAAAAGATAACTAGTGAGAGATCACCCAAGACAGAAGCCATGATCTTACCATAACC
TAACTTCAGAAATGAAAGTCTATCACTTCTGCCATACTCTATGTGTTAGAAACCAGTTACTTGGTCCAGCCCACACTCAAAAGGAAA
GGATTGCAAAAGTGTGTGAAGACCTCAAGAGGAGGGGGGATATCGAGGTCATTTTAGAAGCTTCCTACCATACTCTTGTGGTATGAT
TTCATCCTTATGAAGGTCGGAAATAGGAAAAATGAAATTACATATTGCTTAGGTAATCCTGCCTACATGGAAGCACTATGAAGAAAA
ACGAGGAAATGATAAACATGACATTCAGGGTAATGATGATCCTGGGGTGGGGGGCACTAAGAATGCAGAGAACATACAAGTAGATCC
CACAGGTTGGAGCTGGATGGTGAGTTCATGGTGCTTATTTTCTTTCTGTGTTTCTTGATGTCATTTAATGTTGCATGTAGTTTTGTT
TTTTCTCTTTGGTTTGGTGTTCTGTTGATATGATCAAAGTTACATGTGCATACACTGTAGAGTCAATCCTTCCGTGGTATTTGCTGT
CTTCCACCAGCTCTTCCCATTTACTGTTCTCCAGAGGCAGCTACTTTTACCTTCTTTTGCTAATTAATTTTAGTATTTACCTCCATG
TCTGTAAGTAATATGTGTGTATTGCTACCTTTTGATGCTTTGGTTTATTTAGGTATTATCTATTCCCTTCCCACTCTGGGAGATGAA
AATTTAGCTCCCTTTCCTCTCCTCCAACTCTTCCTCTCCCTTCATCTTCCTTTTATAATTACATCATAATTTTGGTTAAAGCAGTAA
TCAGTGTTTACATTACTCAGTCAACACTAATTTGACTCTATTCATAGTTGAGCCTTGTAGTAAAAAATATGATAACTTTTCCCTTCC
TGCAGAACTTTTCATTTTTCCTGGAGTTGTTAATGATCGTGTTTGTTTGTTTGCTTAGTTTTCTATGTATTCGTCATTAATGCAAGT
GCAAACTCTGGCACTTGTCTAAACCTCTTTTCAGGATGTTCAGGCACATTAAGTTTCCTATCAATTTCATCTCCTTGGAAGTGTCTC
CCAGAGCCTCTTGACCTGCTCCAGCCCGGACGGGTTGTCCTCCGCACCTGGTGTGCACCTGTCCTATGAGGGCCTCCCTGTAGCATC
ACCCTGAGCTCGTTACCTTTATTCCTTGTCTTCCTCCTTGGTTTACTTCTTTGTTTTGGTGAAGCATACCTCCTGTGTCTTCCTGAG
AGAGGGTGCATGGGAGGCAAACCCGTGACAACCTTACGTATCTGAAAATGTCTTCGTTCCACCCTCAGACTGAACTAATACTTTGGC
CAATTCAGATTTCTGGTCTCTGGTTTGGAATTCTGATTTGGAAATAATTTTCCTCCAGAATTTTGAAGGCTTTATTCCATGGCCTTC
TAGCTTCCAGTGTTGCTGTCAAAAAGTCCGAAGCTGTTGTAATTTCTGGTTCTTTGTATTTGGCCTCTTTTATACCCTCCCCCAAAA
CCCCTAAAGCTCTGTAGGATTGTCTCCTTTCCCCTGGTGTTCTGAAATTTCACAATAATGTGCCTTGGTGTGCATCTATTTTCATCC
ACTGTGCTGGGGACAGTGAGCCTTTGATCTAGAAACTCATGCCCTTAAGTTCTGGGGATTTTTGTTTTTGAATGATTTTTAAAATAA
TTACTTCTCTCCTTCTTTCTCCTTCTTTTCCCCCCAGTTGTTTCTTTCTGTAACTTCTACTGTTTGAATTGGGAGACTGATTCACTA
ATTTTCTCATATTTTCTTTCCCTCTCTCTCCTTTTAAGAGATGATGGGGGTCTTGCTCTGTCCCCAGGCTGAAGTGCAGGGGCACAA
TCATAGCTCACTGCAGCCTCAACCTCCTGGGCACGAGCAATCCTCCTGCCTCAGCCTCCGAGCAGCTGGGGGATACAGGCACACACC
ACAATGCCTAACTTCATATTTTCTTTCATTTTCATTCTTTGTCTTTTTCTGCTTTTTGGGAGAATTCCTCGATTTATCATCTTCCAG
CCCCTCTAGGAAGACTTTTTTCATACAGCATTCCATTCTTGTCTCGTGGGTCCAGTATCTTATTTTCTCTCTCTTACGTTAATTATA
GGATTTTTCCTTCAAGAGTTCTCTCTGTGAATGGTCTGTATTTGATCCAGAGTTCCTTCTTTTTGTTTGTTTAATTGACTGGTTTGA
TCTGTGTCTTTCATGTTAGAGACTCCTGACTGTCTGGGGATTCCTGGTTGCCTGTTTATTTTTAAAAGTAGGAGACTAACAACTTTG
AGCTGTAAGGGATTTGAGTGAGCAAAAGTAGACATATGTCTCTACCATCTTTGCCAAGTCATACAGTCTTTTGTATCTCTCATTTAT
TTCATGTTAAGAAGTGAAAAAAAATCCATAGTCCACACCCTGAAAGATAAATCAGTAACTGTAATAACTACCCTTGAGCTCAAAGAA
AAATTACATCTTTTCCTCCTCAGCCTTTAAAGAAAAACATGGAGCTATCCCCCCCCCCAAATTTTCTATTGCAAAAAATAGCACAAG
ATTAGTATGTGAGTTTGTGACATGTGTAAACCGCTCTAAAATTGTGAATAGGTTTAGAAACCTTTTCTCCCTCCTCTCTACTCCCAC
AGTCCTGAATCACCAGCCCAGCCTTCTGGCTCATCACTTCCTGCTTGGTGAGTTGCATGTATGTCTTATCTCCATGACGAAGCTCCA
AGCCACTGGAGAGCAGGGTGCATGTCCAATTCATCTCAGCATCTTCCGTAGCCCCCACCAGAGAAGCGATCACTGAAGGCTGACAAA
CAACGAAGGACACTCTCAAGATCTTCACTCCTCTCCTCAGCACACACAGTTAGCACGCACAGGAGTTTATTATGGTGAATGAAAAAA
GAAACCCACAAGAACATAATTTATATGTAATTCCACTTTGATCTCTAATCTCTTTACCTTGGCATCCTCCCACATATGAATAGGATG
GTCCATGATCCTAAGGGCAGCCAAACAGCCAGGGAGCACGAGTTTAATCCTCAGGGTTTGCAGGGAAAGGAGGACTGTGGAGACCCA
CGTGCACAAGAAAATCCATGATGTGCACAGAGGGCAGGCTACAAATTAATCCTAACTTCATGGTTCGGTATTCAAGGCTCTCCATTG
CTTCTCCCTGGACACCTGCAAAGCTTCTAGCAGCACTGCTTATCTGGGGCCTGACCCCTTTAGTCAGTCTTTCATGTAGAAGAATCA
TTTTCAGATACTAAGTATCTCATGTCTTTTCTTTGTCCAAGAACTTACAGTTGATTTCTTTTGTGTCTGTATCAAATAGTTCTCCAG
CCTAAACTTCAAGACATCTTCATGAATGCCTCACCACTCCACTGCACGTGTAAATCCATGCCCCTGTATTTTTCTGTCTATTGTTTC
TCAGACTGATAACACCTGCGCCCTCTCAGCAACAATAATGGCTGACATTTAGTGAGCACTTAATTTGGGTCACTGTGCTGGTATTGC
TTGTGGGTTATTTTACTTAATCCTCCCATCATTGGATAAGGAAATTGATTGACAGAGAGGGCACGTCACTTGCCCACAGCCATGGAG
CTACTTAACTCTGGAGCTGGAGCTCCAAGCCAGGCAAGCCCAGCCCAGCCCAAGATCACTGGCTCAGCTCAACTCCTCACTGCTAAA
CTGCCTCAACCACATTTCTTCAGACCTTACCCAAGACTTATCTTCCAGGAAATGTTTTTCGGAAGAACTGAGGACCCTGGCTGTGAC
TGTATTACATTTATGTGTTGCAGGCTTGTGGCGTGGAGCTGCCTTTACGTCTTCTCCCTTCTGTGTCCCCTCCCACAGTCACTGAGT
CTTGCTGCTTTAGAGAAGCTAAAGTCTGATTTTCCACCACTGCCTCAGCACAGGGCCTGAGGATAGGGTCTGCCACCATCCCTCCAG
CCAGAAAGCAACCTGCTGGGAAGGGAGGGGTTGCATTGACCTGAGGGCCTAGCAGGGCAGAGTGAGCAGGCCAGGGAGACAGCTGCC
TTTCCAAGGCCACAGCAGAACTGGGAGGGGGCTGATGCGCTGGGTCACGGTCAGGGGTAGACGCAATCCTGGGAATCAGAGTTGGGA
GCCACCAGCCTGTGCTACTCAAGGATAGCCAGGGGAGCAAAGGCAGGCCCAGCCCAGGGAAATGAGGCCCACGCAGCCAGCCAGGGG
CCCCTAGGGTGGTAACAGAGCATCCCTGGGGAGCAAGTATAGCAGATGGAGTGGGCAGGCAAGGCCATCTCATTAGCAGAGCTTTGG
GGAGGGGGTATCTTTTCTTCAGATGTCCCCAGAAGTACCAGACACCCATGTCCTTGTGCAGACAGCCACAAAAAGGCTAATGCTGTG
CCACCCACTGGGTCTCCAGTTGCTGGCCAAGGACTCAAGTGGGGCTGGAGAGCCCACCATTGATGTGCTCGCGGGAATCTGCTAGGG
CCGGGCTCCGGTGGGTGAGAAAGGCTGTGGCCTGCGTCTCTGTCCCAGCTTTGCACACCTGTTTCTTGCCTGGTCACCCCACCTCTG
TGTCCTGTCTCTTCAGATACCCTGCTCGCCACCCGCCGACCATTCTTCCCAGCAAGCTGTGTTATCTTGTCATTCCTGTGCTCAAGA
ACCCAGAATCTCTTCTAAGGCCTCTAAGACAAGATCCAAGCTTCTTCTCCCAGAATTCCAGGCCTTCCTAAATCTGACCCCTGGTCT
CTGAAGCCCCCTGGTCACCTGCTACTGGAGCCCAGGTGCTCTCCTCTCACTGAAGCTTCCTCATTATTCCCAAGACACACGCCAGTC
GCTTTTGCCTCTCTGATGTTGAGTGAAACAGTGAAAGGAGCATGGGCTTTGCTTCCTGTGTGTCCTTAGGCAAGTCACCCAGTGTCT
CCGTGCCTCCTGCTGCTTACCTGGGAAATACTTTAAAGACTGTTGTGAGATGAGAATGTTTGCGTACAAAGTGCCAGCCACACAGTA
GGTGCTCAATACATGCACAGTATCCGTCATACCATGCTCTTGCTGCTCCCTGACTTGATGGCCCTGTCCCTTTCTGACTGGCTGAGC
TCCTGTCTCCTTCCAGGCCCATCTCTAGATGCATCTACACATGCAGCCTGCCCTGCACGCCTCACTTAAGGCGCCAAGGGAAGGAAA
AGCAGCATTTGCCTATCACTCGCTGGGCACAGACCCTGTGGGGGCATGGAGATTCCTGATGACGGCAGCGAATCCTTCCAGGGGGCT
TCCTTGTACTAGGCCTGTCCCGGCACGTTGTGTGCATTAACTCACGGAAGCCCCAATGGTAACTGACTTCCATAGATTAAGGTTCCT
AAATGGCTTTTCTGCTCTATCCAGCCTAGTAGGTTTGGTCTCCCTGGCCCTTTGTTCTTAGCTTTCCCCATCCAGTGGCCTCCAAGT
GGTTAAGAGGAGATTTTATCAAGAGGTGAAGCCCTACAGTATAAAATACAGAGTCTATTGTGTGTTGTTCAGGCTGATCCCATTAGA
GAACGGTTTGGAAGAGGACTCACCTTCACCAGCCCTGCCACCCAGCCTGCAACCAGCTCCCTTGCTGCACTTTGTATAAGCCCTGGG
CTTTCATTAGGGTTCTCTGCCCAGAGCACCCGTTCCTCTGGGCCTGCCTGGAAAATGCAAGCCTTGCTCCTCTGTGGCACCTCTGTG
GACCCTCATGTGTTTGGGTCAAGGTCATTGCCCTCTGCCTCCATCCCCAGGGCACCTTGTCCACCCCCTGATTGCACCCCTCTGCAG
ATGTCTGCCTCCTTGGCAAACCCCTCTCTTGGGCTGGGTCCCTGAGGGCAGGGGCTGTGTGGTATTCATCTCTGTATTTCAGTACTT
GGCACAGAGCCTGGCATGCAGCAGGTCTTGGTGTTGGGGATGAAGGGAGGGAGGAAGGGGATGGGGTGGAATAGGCATCCGGGCAGA
GGAAATGGTGCTGATGTTTCCAGTTGGTTCCTCCCAGGGCGGGGCAGCTACAAGCCCCTGTCTGACTCAGAAAGGGAGGCCTCTGTG
GACACCCCAGAGCCCCCTCTAGAGACAGCTCCACGAGAGCCCCAACCACCACAGCTTCCCTCCAGACCCCCGGGAGATGCCAGTCTT
TGAGATGCTTCAACTTTCTTAGAGGAAATCGCTGAATATGTTTGGTAATTTTTTTTTGACATTTTCCCTGTTCAGGAGCTTGTGATT
TCAGATTAATCACATCCACATACATTTAGACTGTCGGCTTGGAAGGGAAAATTTGAATTTTATTACTGCATGATTTCATCCAAAGCT
ATCTTACCAACACTAAAAGGATGTCTACAATGAGGTGCAAATCATGACAAAACATTTTATCTCTGGGACCAAGCTCATGGCGGGAGG
CCAACGGGACGGGGGGCTGCAGGTGGGACCTGGGTGGGCACAGTTGCAGCCCCTTCAGTAGCCTAAAGAGATCTGTGTGAGCCACCG
TCACTGCTGCCTCTTCCTCAGCAAAATATCTCATTTCCTTCCCCGCTCTAGGTTTGCCCACAGCCCTTACTCCCAGGAATGGCTGTT
CATCGACTAACTCCTGTCTTCCCATTGGCAGGTACCATGACCAGCAGGACGTAACTAGTAACTTTCTGGGTGCCATGTGGCTCATCT
CCATCACATTCCTTTCCATTGGTTATGGGGACATGGTGCCCCACACATACTGTGGGAAAGGTGTCTGTCTCCTCACTGGCATCATGG
TGAGTACCCGCCTCTCCCCATTCCCTACCCTTAAAATGTGGCAAAGAGCTGAACTCTTTCTTGCAAGCACATAGGGGCCTTCCTAGC
CGCAGAGCGGGGGGTGGCTGTTAGTTAACCGACCTGCATGTGTGTCCCCTACACATAAAATGTCAGGTGGGAAATCTGCAATGGTTT
CTCACTTTTCAGTTAACTTTTCTAAGGTAAGAATGAGACCTGGACTCCTGGCAGTTAATGGTCTATTCACAGCTCCCTCAAAATGGG
CTGGGTGAAGCTGGTCCTGGCCCAATGCTCTGATGCCCACCCCACTGCTGTGCAGCCACCCAGTTTGCTCACCTCCAGTGAGCACTG
GAGCTTGCAGTTTATTGTCCTCCTTCCTGTTGCCTTGGTCCCACTGACCTTGGCACCATTTCTGATCATGTTTAGCTGGAACTCACA
GAAGCAGGGACTGGGGAAAGAGCACTGAGTCGGCCTTTGGGACCTGAGTCCTGGACGGGGCTGTCCACATGGCCCTGGGCTGGCTCT
TGCCTTTCCCAGGCTTCTGCTTCCACCTCTGCCTATGGAGGGGCTGGGCTCTGAGTGCCAAGTGCTTTGCAGCACTGGCATTTGGCA
AGGCTGTGATCCCATCTCCAGGTGCCCAGCAGGTGTGCCGTGCCCTTTCAAGGTGTATCTCTCCTGCCCAGTGCCCTACCACCCACA
CTCGACACTCTCGCGGAGGCACACACTCTCACCTGTCACATGGCCATCACAGTGTGGCTTATGAGAGGGATGTGGGGTCTGAGTTTC
TGCTCACTGTGGACCCTGGGCCAGGCACTCTGCATATTGGACCTGAGCATCCTCCAGGGGAAAGTGGAGATGGAGGGCCTGCCCTGC
CCTCCCCATTGGTTTGTGCAAGGACCAAATGCAATGATTTATGTTAAAGTGCTTTGAAAACGAGAAGCGGCCACACACAGGTCAGTG
TTGGTGGTGTCCTCAGTGTCCTTCAGTTGCAAATTGGTGAAGTGTTCCCTGGGCCTCATTGCACTTTGGGTCCAGGACAGGCAGCTG
CTGCTCTTTGGCCTTCGTCCTGGAATCCTAGAACAGTGTCAGACCAGGGTTGGACACTTTTTTCAGAGGAGGAAACTGAGGCTGAAA
GAGGAAAGCACATCTAAGCCTGGGTAGTGTTGGGGGAGGTGGCGGGGTGAGAAGTGGTCAGGCTGCTTGTGGTGTGGCTCTGGGTCT
CCATGGCTGGAGACCGCAACCCTCCCAAGGACAGCCCCCCGTCCACAGCCCCCCTCACACCTTTGTGAAAATCCTTCCTTGCAGTGA
TCCCTCAGATTCCATTTGTCACCCCCGTTGCCTCCAGAAGAGATGAAGACCCTCTTTCTGGAAGGGCAGTGACCCCTTTCTGCAGTG
CAAAGGGAGGGCCGCAGGTCTTCCTGCAGGGTCTGAGTGAACGCCTCACAGCGGCTGGGGGCTGCCCCCACTCCCCACCCCACTCCG
TCTTGTTGGCTTCCTGGGGCTGATATTGGTGGACCAGACGGTCAGACCCAGGCTTGGGGCAGTCAGCCTGCTGAGCAGGAGGGGCCA
GGCTCTCTCCTCCTTTCTGACTCCAGTCCAGGGCAGGTCCCTATTAGCAGGCAGGTCCTACGACAGCAGCTAATGACAAAAAGCTTA
GCGCTGGGGTTGTGATAAATCACGGGTAAGTGCAAGGCTTTAGAACAAATACCTCCCATCGGTTAGTAGGAGTCTGTCTTGGCATAG
CACCAACTGTCACGAGGAAACCGGGGAAAGACGCTTCTTACCAGAAAGGAATGAAATGCCACTGAAGGCCAAATCTTTCCTTCTTAG
AGTCACTCCTGGGGCTTTGTGCCTCAGAACCAGCTCTCAGCCAGCGCTGGCACAGAGCAGGGTTGCCAGTAGGTGGATGGGCCCTGC
CGGGAGCCTCTGTTATTTTAAAATACCGACCTTTTCCCCTCTCAGCACATAGCTGTCCCCATCCAAGAAGCAGCACCCCAAAGACCT
GGCTTCCTGGTTCCTGGACTCCGCATCTCCTGGCTCTACTCTGGCACAGCTGAGCGGAACAGGCGAGACCCTGGCCCCACCCCTGCC
TCTCTTTGGGGTCCGGAGGCTGTTTGTCTTTCAGCACCCCCTTTTGCTGTCTGGGTGTGCAGTGCTCCCCCAGAGAGAGCGTTCTCT
TCCCTGTGGATTGAGGTGTGATCAAAGTGGAAAGAAGAAACGGACGATGAGGAGCCAATTCCCACCTTCCATATGAAAAATACAGCC
CAGGCCTTTCATCTCTTTTTGTACAAAACCCTGTGCATAAATATACACCTTTCTATATACAGGAACAGGGAGGTTTCCTCTATTTAT
TGTCAGTGTTTTTCACCCAAATTAAAGTCAGTCGCCTCACTCTGGGGTCAGCCACAGTGAGGAACCCTGGTGGCATTTTTCTAACAA
ATGTGATTTTTCTAGCTGAGTCACTCCTAGCCAGGGTTTCACTTTGCTGAGGATCCTTTCCCCTCCTCCTCCCAACCTCCCAACACT
ATGTATAGCTCCCAATTATTATTGCAAGTGCCTTTACTAAAGGTAGGGCTTCCTGTTTGGTTACCATGGACACCAGGAAGGCCAAGG
CAAGGCCTGCTTGGGAAGGGAAAGGAAGGATTTGTGTGTGTGTGGCCTGTAAATAGTCTTAATCATTGTTTTCTCTTGGCAATCCCA
GTAACAATGTAATTATCAACCATTAAGCAGGGTATATAATTGTCTGAACAACTTGCCATGGCTGGCACAGACCTGGTGTGTTTGAGA
GATTTCACCCACAAAAGCAGCTACATTAAAGTCCAGCATCTTTCCATAAGGATTTTTCATGATCATGGTGACAACACAGTAGAGAGA
AAGCTGTCAATATTTAGTATCCAGAATTGGAAATGACAGTGCAACCTAAAAGATGTGCAATGCTGACCTTCTGATTCTGTAGGCATC
TGGAAGACCCCAAAGCCCCCAAGGCTGCTTGGCATAACTTTGTTTCAATCTAGCCTCTTTCTAAAGCTGGACTCTCTCTGTAGACCC
TTCATCAGTTATGGACAAACAGGCAAAAGCTTCTGGATATCTGCAGGGACGCTGAGCTTTTCCCCAATGGCCTCATGAGAGATGCCT
CTGAACTGCCAAGGGAAAGTGATGATGAAGTAAAAGGTAGCACAAATTGAGTCATTTCGGGAGGAGGCAAAAAGAGGGCTCAGCAAG
ACATCTGCTTAATGTCAGTTTTAGAAGATGAGCTTCTCCCGAAACACCTTGGTTTCAAACCAATTGATGTTATTTTAGTTGTTGCAT
GGAATCTGTTGCAGAAACAGAATCCAACTGTAAATCCTCCAGGCTTCCCTCCTCCATTTGTCACTTTCATTTGTGTTTTATTTTATT
TTATGCTTCTTTAGTTAAGAACACCTCATACTTTTCCCCTTGGGATACTTGTTTTTCCAGCTTCTAAGGTTTTCCAGCTGGAAAATG
TTTCTTGAAAACAAGAGGTGGGAGGACTGGTGTGGAGGAGTCTCGGTCCAGATTTGGGTCCGAATCTCTAGAGTTAGGCGTGTTCAC
CCCGTCCCAGCCACCCTCTCTCCATGCACTCGGTGCTCCCTCTCGCCCACGTGCCTTGGGAAGTTCCAGCTCATCAGAAGTGGACCC
CATCCACTCCCCTCGCCCCATGGAACTGGAAGTTGGACAGAGCACATCCAGGGACTCCAGTTGATGTCCCCTGTGGTTCAGGACTTG
GCATATCTGAAAGCAAAAGGCTGAGTCATGGCTCTTCCCCCCAGTCTGACCCCAGGGAAGTCATAGCAGCAGCTGTTTACCCTTTCC
CATCCTGCTTCCTCCCTCCTCACACACCCCCTCTACCCACCAGCCATTTCAAAGATTCCACAGTTAGCCAGGCCCTCGGTGATTTGT
CTCTTAAAACTAACCTGCATCACTGCCATGTGACACAGCACTGCCAGGCTGCCACTGTGGCCTGTCAGACTATTAACTCACCTAAGT
GAAAGGGCCCAGCATCCCAAAGGGGACCCAGGGGACAGGCAATCCTTATACCACTTGAAGATGGACACAGCAGGGTGGGATCCCTTG
GCACTTCCTTCATGTCCTCTAAAGGGCCAAAATGGACCATTGACTTCTGGCTCAAGCCTTAGGCTCCTTGTTCTTCCTTCCTCTCTC
AATCCCATCAGCCCATCATTTTTTCTTATTCTTCAGCCTCCTCCTTCTCTCTTTCCCCCCTTGACCCTTGCTGTTTAGGATGCAACG
CAACTTGTTCCCCATCTCATTTTCTGCCCAGCTTTACCTCTCCCTGCACCCACCTTCTCTTCTCTTCTCTTCTCTTTTCTCCTCTTC
TCTTCTCTTTTCTCCTCTCTCCTCCCCTCCCCTCCTGTTCTATTGTCTTCTCTCCCTTCTTCATTTTCCCGCCCCGCACCCCGCACA
GTCTGCAGAGGCTCAGCAGTCTAGGCTTCAGGATTCCTCCACCACTTTATGAAGGCTGCTGATGTCTGGCCCCATCTTTATTAGGTT
GCCTCTTAATGGAAAAGACCAGGTTAGGTGTCATTACTGGTGACTGCCCTAATTACATCCAGTCAATGGAAAAATGAAGCTGAGAGC
ATCCATTTAAGTTGGAATTAAGGGTTAAGAATGCAGCATCCTCTAGAGGAATACTCACAAGGGAGTCATAACATCTCTATGTTGAAA
GAAACTGGGAAGTGTCATCTAGTCCACTGTCCTGCCTCCAGAGAGAAATTTATCTTCATCATGTGAGACAGGTGGTTCATTAGATAA
AACACTCTAGCTTCTCATTAAAAGCCCCCCCTGCAATCAGCCAACTAAAATGTGGGCCATGGCAAGCGCTGTGAAACACCCCATCTA
GACCCTGGAAATTTGGAATGGTGGATGATTGATTGTTGTGATTACATTCCCAAAATAGCATGGAACAGGGAAGCTCCCTCTTCAGGC
CTATTGGACCCCAGCTTTCAGGGATGATTTAAGTAGAGTTTTGCCTGGGCCTAGCGAGAGGAGGGAGAATGGAACCTGGGGAGAACC
CTCTCCATCTGATGTTCCATTTTTAACGCTATGATATAGCCCTCTTTTTGGGCTTTCAGCCCAAAGTACTTTTGGAACTCCATATAA
AGGAGAGGGATGATACTCCGTCCGGTCGAGGGGAAGGGAGCACACATCTTCCCTCTAGGATGGCTTCTTTTAAGCTATTTTTTGCTT
TTGGAGGCATGGCTAAATTGATTACAGCTACAGACCAGGCTGTGCAACTGCCCAGATCCTTTGAAAATTGGGCACAGAGTCTCCTGT
AAGTCACAGAAGCTGAAAAAACACAGCCGTCCTCGAACAAAAGAGGAAAATGAGGTCAGAAAAGGAAAGTTCACACAGACTTCGTTC
TTTTCACTTTGTGACAAGGCCAACAAATGTACTGAGAAACACTAGATTGTTTTAATGGGGACCCTTGTGGGAAACGAGGAGAAAGCA
TTGTCAACTTCAACCACATTGAAACTTCACACAGGCTAGCCTTTGTCAGCCTGTGGGATAAAGAGAGAAATAGCTTCTAAAAGAGAG
AGTAGGGAATGGATCCACCCATATGCATTCCTTCCAAGTCGGTTATAGATTATAGTACCTTTGAAAAACATTCTTTTCTGCTTCTCA
CACATTTTGTAAGACCTGATGAGCCAAAAATGTTACTGACAGATTCCTTCCATTCTGGGACCTCAACTGCTAGACTGGGTTCTGTGG
CAACTAGAATTACTGTTTGACATACAGGATTCGGTTGTTGGCTTTAATAAGTTCATCATCTCTTTCCATGAGTGGTGGTCAGGGCGG
CGCAGGGCACAGCATGCAGGCAGAGGGCGATGGTCTCTGACGGGAGTCCGCTTGCAGAAGGATGGAGCTGACTCCCGCTGCCTGCCG
AAGAGCCAGGGGAGTAGTGAGTCTCTGAATTCCCCCTATGCTCACAAGCCAACCAAGAAGCCTAGCGAAGAGGATTTTAAAATGCAT
TGGGAGAATGTTGGAATGTGCAGGGAATTCCCTGCTTATTTCCCAGACAGTCAAGTATGCACCCAGAGGATTAGGAGAGAGAAAGTG
AGTCCCACCATTCTGGGGGCTCAGCAGAGCCTGCTTCCTTGCCCACCCTCCCCCGGGCTCCCCTGGCACGGACCTACCGAGCAGACT
AGGATCGCTGTTCCAGAAAATCCCAAGGGTCTGCTGCGGGCTCACTTAAAATCCCGGTTTTGATCCAGCAGAAGTGGTGTTGTCTTT
AAAATCTCTGCATGACGGTGAATTCATGAGCCTCCTGGTGAAGGCAGGAAGGAACCGATTCCTTGTGCTGAGTCATAGCGTAAACCT
TGAGGTGAAAAAGCCCAAGTGTAGAGCAGGCACAGTCACTGAGTGCACGTGTTCGTGCCAACCTGGGTGCCCTCTGTGAAACAAGCC
CTGGGGAAAGGTATTGCCTCTTCATTCGATTATTTTCAGGAACACTCTCAGCCACTGGCAGGTGTTCCATCTGCACCCCAGCTGATG
GCCTACCTTCTTTCCTCCCAGGCCTGCTGGTCTAGAGCTGGCAGTGGGATGGAGACCTGCCTGAGGGAGAGAGGGCCCGTGTGTGCT
GGCCCCTCTGTCTTCCGCTGCACAGCTTGGCAGCTGACCAGGTGCTTAGTTGTTGGTTAAACCCCCCAAAGCCTCGGCGCCACCCAG
CATCCTTCTCCATTGAAACTGGCTAGCTCAGCCTCCCAGAGGTCAGCTGCAGAGCACAACCCAAGAGGCACTGCAGACCCCTCTGTG
GGTCAGGACATCAGAGTCCTGACAGATTTCAACAGATTTTTGTGGCTTGGTCGTGACCAGCCAGAGAGCTCAAGTTCCCACTCCACT
CCCAGGAAATTACCCCCGTGGCCGCCAGGCGTGTTGAGTCTCTCATCTTGCCTCTTAATGATATGTTCTTTCCCCTCTGGTCCTCTT
GATGTCCCCCCGCAGGGTGCAGGCTGCACTGCCCTTGTGGTGGCCGTGGTGGCCCGAAAGCTGGAACTCACCAAAGCGGAGAAGCAC
GTTCATAACTTCATGATGGACACTCAGCTCACCAAGCGGGTAAGATGCCACACAGCCATGTCTTATGGGGGACTTAAGGCCACAGTG
GAAGCCAATCAGAAAGCAAATGGTCCAACGGGTGCAGTGGCTCACGCCTGTAATCCCAACACTTTAGGAGGCTGAGGAGGGTCGATC
ACAAGGTCAGGAGATCAAGACCATCCTGGCTAATACGTTGAAACCCCATCTCTACTAAAAATACAGAAAATTAGCTGGGCATGGTGG
CACACACCTGTAGTCCCAGCTACTCAGGAGGCTGAGGCAAGAGAATTGCTTGAACCTGGGAGGCGGAGGTTGCAGTGAGCCGAGATC
GCACTACTGCACTCCAGCCTGGGTGACAGAGAGAGATTCCATCTCAAAAAAAAAAAAAAAAGAAGGAAGGAAGGAAGCAAATGGTCA
TGTCGTATGGGTTTGCACCAGGCTTTTAAAGGGTGTGTTTGAGGGAACCCCAGACACAAAATGGAAACAGCACCTTCATACGCCGGG
TTCCTGTCTTCTGTTCGATCACTGTGCATGGTTCAGGCCAGCCAAATATTTTCCTGGTAATTTAATTAATAGCCTACAATGGTATAA
CCTGCCCCTTTGCCTGATGATACCAAATAAATCACGGTATGAGGCAATGTTTTTTGTTTATGCTCAAATATTTTCAGGATTCTCATT
AGGAAGGGCAAAACTATATTGAAAATATACCTAAAACTATAAAAGTATATTGAAAATGTACCTAAGTCTAATCCTTTAAAAAAAATC
ATTTAAAGAATCATTCTATGCTGGGAGCGGCGGCTCACGCCTGTAATCCCAGCACTTTGGGAGGCCGAGGCGGGCGGAGCATGAGGT
CAAGAGATCGAGACCATCCTGGCCAACATGTGAAACCCCGTCTCTACTAAAAATACAAAAATTAGCTGGGTGTGGTGGCAGTCACCT
GTAATCCCAGCTACTCAGGAGGCTGAGGCAGGAGAATCACTTGAACCCAGGAAGCGGAGGTTGCAGTGAACCGAGATCGCGCCACTG
CACTCCAGCCTGGGCGACAGAGCAAGACTCCATCTCAAAAAAAAAAAAAAAGAATCATTCTAGTAGAGCCCACATCCCTAAATAAAA
TTGTTCATCAAAACATCATCATCCAATTTTTCCCTTTTGGGTTTTCCGGGTAATGTTTCTCACTTGAACCATTATGGAAGTGGTTAT
GGAAGATGAAAATCTGAAAATATGCAGGAATGGGTAGACCATTTAGCTGCTGAAAGTTGGACACCCCACTGAATTGTCTGAGATGAA
AAGACCCATTTAGAATAGACAGATACAAGCCTTCCAGAATAACAGCAGGCCAAATGGGCAAGGCAGGAGCTGTTTATTCAAGGACAG
CTTTTATCGAAAATTCTTTAGAACTACAGCCTGAAGGAAGGTCAGTAAGATGATTTCCTGCAAGACTGTCCTCTTTCTGAGACGAGA
CAGAATTAAAACCCTGTGCCCGTGTCTCGGCCTCCATAGTCTTTTTCATTTAGGACTGAAGTAGGTTAAGGGACCCATGAGCAACCA
AATTTTGATTTGGGTTTTTGAAAGCTAGAGAAAATATTCAGTGATTAATGTTGCACAGCAGCAAAAATGTGTTAGAAATAGAGACAA
CGGCAGGAAGGAAGAACAAAAAGAGAGAGAGAGAGAATGACAGAGGGAGCTGCGAGCGAGCTCTTGGGGAAGGCAGTGGGAGAGTGT
GCAGCGGCCAAGGCCTCTCCAGTTTCTCAGCAGAATTATTTATGGTGTTTGATTGTTTGGTCTTTGATTACATTTATTGGATTCTGT
AAAGAGCCTTAACAGACATTAAAAGGAACTGGTACGGGCAAATATTCTTATGTTGTTGACATTCAAGTGTTTGTACTTGATACATCA
TGGCCCTGAGCTATCAGCTGCACCAAAAAAAGCCATGATTTGTCACATCACGATGGCAGTGATTGAAGGCTGTTATCACAGTAAGGC
CTGCCGGGAACCCTGCCACTTGGCCACTCTCTTTCAGCCCCCTCTAATAATGGGTGCAAAAGTCATGCTCAGTATTGAGGAAATTAT
TCAAGTTATTGAAACACCTAAATGCAAATCCACTTGCATCTATTTTCCTGAAGCAATTTACTGAGGTTTCACTGGATTGAAACTACG
TGCCGGAGTGAGGGCAGGGAGATGTTTTGCCTCCAAAGCCTTCTCATAAGCTGTTTACCAAGAGTAGAGACATTAGCAGTCTCTCTG
TTTATTCTGGTTTTCTTTTTTAAGCCAAGAATATTCTTTGTATTTGGTGTTATTCCTTTGTCCTCTCCATTTTTCACCAAGTAGATC
AGACGCTAGAGGAAAATAAAGCTGAGGAAAGCCAGCTCCTTTGTATGCCAGGAAAATAAACAATTTTTCTGCAATATAATTGTAGAA
GATGAGACTGTGGTTGCTAATTTTAATCTTTCAAAAATGATTCCCTAACATCCTCCTCAATTCCACTTCTTGAAAAGTAGGACTGAA
AATCAAATCAACAAATATTTATTGCTCACCCATGAATCTAAGGTAGCTTTTTAATCGAATAACATCTCACTTTTCTGAGAACACTTA
AATCTGTTGAAATGTCTCTTTAATATCTAATATCTTTTCTCAAGGAAATAAACAACACAGAAGGGATGGTTCTTACAGAGAGAAGGT
TTTCAAAGCTGGCAGGACTAGAACTTCTGAAGGTTCTTTTACATAACCTTAATTGAAATGAAACTGGATGTGTGCATCCTTGGCCTG
CTCTGGTTTGAGCTGACTTTGACCTGCTCACAAGAGAACCCCACCTCTCTTCCCCTGGGGATGCCTGGCCTGCCTTCTGTCCTCTTG
AAATGGTCCATTGTATCAAGCTGGAGTGGCTCATTGGTCAAGAAGATGCTAAGCAGCCGGGTGCGGTGGCTCACGCCTGTAATCCCA
TCACTTTGGGAGGCCGAGGTGGGTGGATCATGAGGTCAGGAGTTCGAGACTAGCCTGACCAACATGGTGAAACCCTGACTCTACTAA
AAATACAAAAATTAGTGGGGCGTGGTGGCACACACCTGTAATCCCAGCTACTCAGGAGGCTGAGGCAGGAGAATCGCTTGAATCTGG
GAGGCAGAGTTTACAGTGAGCCGAGATCCCACCACTGCACTCCAGCCTGGGCAACAGAGCAAGACTCTGTCAAAAAAAAGAAAAAAA
AAAAAAGAAATACTCACTCTCAGACATGATCCTTAGAAGCTCTTGGCCGGGCGCGGTGGCTCATGCCTGTAATGCCAGCACTCTGGG
AGGCCGAGGCAGATGGCTCAGGAGGTCAGGAGATTGAGACCTTCCTGGCTAACACAGTGAAACGCTGTCTCTACTAAAAAAAAATCC
AAAAAAATTAGCCGGGCGTAGTGGCACGTGCCTGTAGCCCCAGGTACTCGGGAGACTGAGGCAGGAGAATCGCTTCAACCTGGGAGG
CGAAAGTTGCAGTGAGCCGAGATCGTGCCACTGCACTCCAGCCTGGGCAACTGAGCGAGACTCCATCTCAAAAAAAAAAAAAAAAAA
ATGCTAAGCTCAGTCTATTCATAACATCTCAGTGAATTCTCAGAGAAAGATCCAACATGAAGGAGTTGACTGTGGCATAATTGCAAC
CTCTTCGGAGTGGGCAATTAGTAGATTTGACAGATAAAAGCAGTCTCTTACCGGTATATGCAACCTTGATGCTTCAAAGACCTACGC
AAAACGAAAAAAGGAATTGTAGCTATTAATCATAGAAAATACTCAAATTGTTCTAGTATTTATTCATCCTGTCTTGCTCTAAAGACT
GCAGCCTAGCCTTTGCCATGAGAGCCAGCACTATTTTTATTTATTTGTTTGTAGGGAGGGAAGAGGGTCTGATAGCACTGGGTAAGA
ACTCAATTTCTGCTTTTCTTGAGTCCCATGCTTATCTTCTTTCCTCGTATGACTTATCAACTCTGGTGGCAACGTCATTTCTGTATT
TAATTCAATCAGATCTTCCCACACTGACTTCGCAGTGTCACTGCCATTGTTATTCTGTAATTATAACTTATTGCTAATCTCTGGGCT
ACTAGATAATATTTAAGTACATGATTCACAGAGGGACTGGAAGAAAGATACTAGAGCCCCATCTTTTCTGCCACTTAGTGTTTTCAT
GTCCCCATCTTTCTGTTTTATGTAGCTAAACAGATCCATCACAGAGGCCACTCCCTGGGTGCCACACTCTGTACTCTGGGGGCACCA
AGATGGGTAAGAGGTGGTCCTCACCTCCATCCCTAACCTCTGGTGGCTGACTGCCCTCAGAGAGCACTTCTTCTTATAAGAGGTAGA
CTGGGCCGGGTGCGGTGGCTCACGCCTGTAATCCCAGCACTTTGCGAGGCCGACGTGGGCGGATCACTTGAGGTCAGGAGTTCAAGA
CTAGCCTGGCGAACATGGTGAAACCCCGTCTCTACTAAAAATACAAAAAATTAGCCGGGCATGGTGGTGGGTGCCTGTAATCCCAGC
TTCTCGGGAGGCTGAGGCAGGAGAATTGCTTGAACCCAGGAGGCGGAGGTTACAGTGAGCTGAGATCGTGCCACTGCACTCCAGCCT
GGGTGACAGAGTGAGACTCCATCTCAAAAAAAAAAAAAAAAGGAAAGAAAAAGAAAAAAAAGGCAGAGTGGGCTGCTATGGACACAC
TTGGCTTGGGAGGCACCATGGACCGGAGATATTTTCTTTGCGGTACCTACTCTCCACATCAAGCCACAGTGAGCAGAACCTTCAGAC
AGCACAGTGTGCTAAGGATCCTATCTCCTTCTTGCTGCAGGGGTTTCTTGCCCCAAGTAGCTCACCCAAGTACACTTCCCCATGAAT
TCAATGAATAAGACCAAAATTAAACCCACAACTTCCCTAAACTATTTCCTTCTCCTAACTTCCCTGGTGTGCTCCTGACATCGTGTT
CTTCCAGACGCCAGGCCTAAAGACTTAGCCCCATTTCTGACTCCTCTGGACCCTGGACCCCACGAGGGTCTGAGCACTTTCTGTGTC
TGATTTCACCTTGCCGTGGCTGTTCACGCATGCCCGCCTCTCCAGGGCCTCGGCCCCGCCACTCTTGAAGCCGCCATCTTCTGTCTC
GCACTATCACAGCAGCCTGCTACCTGCTCTCCTCACTTCGGCGTCCCTTACCCCAGTCATCCTGCACTCTGCTTCCCCATGGATCTC
CTTAGACTCTCAGGCTTAGCTCTGAACAAATTACTTCCTGCTCAAAAACCTTTAAGTAGTTTCCCCACCTACTATCTCCTGAATGAC
ACTGAAGAAAACTTTTCTTTAATTACAAGCATATGCCAAAGTTTGATCATAATCTTGCTAGAAGAAAATTTTCTTCAGCAGGCTAAT
TCAAAGATTACTCTTAGTAGTGGTATTTCGTAGGAAAAACAATGGTAGTCATTGTTATGCCAGATTATAGAAGGTTTAGCTAAAGAC
TGCTAATGGACAGCTGGTTGGGCTCAACAGTGGATTTCCAGAACAAGTCTTCAAGTCTTTACAATGGATCAGATAATCAATAGGGCA
GTTATCAGGAATGCTAATTCTCCCCAGAAGAGGATTATAAATTACATCACGCACAAGGTTCTGTCCCAGACAGATTGGGTGCAGGTT
TTCTTTAGCAATAGTCACAGAAACAATGTGCGTTTAAGCTGGAAGGACCCTTAGGGAAGAGCCACTCCAACCCACATGAGAAGACGA
AGGCCCAGAAAGGGGCGAGGGTGGGCCCCCGGCCTATGCAAGATCCACTTTGCTCTCAGACCATCAGGCAGGCAGCTTTAGGGCGGT
CAGTTCCTTGGAACTCAGCATGGGGTTAGGGTCCCAGACCTGCTGAAAAGAAAAGCCAGCTTAACTCCACCCTAACTGAGTATCACA
TAAATAGCATTATAGAATTATCCACCACCCATTACCATATTGCTACAGGGAGAACCTCAAGCTCCAACTCTGAACAACGGAAATACA
CCTCCTCTCTCTCTACCCAATTTCCTGCCAGCCAGAGAGGTGGAGCTCCCAGTTCTAAGTGGTCAGTGAGGGCCTCTGGGTGAGGCA
GGCAGAGAAGCCTGTAAAGGGAGTGTGGAATAGAACAGGGGGAGGAAAAAGAGGAGGTGCAAGCAGGGTGATGAAAGAGAGGGCAGC
CGGGGAAGCCAGAGCAAGTCTGCAGGGTGGTGATGAGGGGAGAGTGGGCTTGGCAAATGGGAAAGAGGAACCAGCCCCCTTCTCTAG
TCCTGTGTGTAATGGACACCTTCAGTCAAGGGCTGGCTTGCAGGTTCTCCGATAGTGATCTGATCATCACTCAGCCTAATTTCAACC
TGTGTAACTCCTTGAAGTTACAGTCATGCACTGCATAACAAGTTTCAGTCAATGATGGTGCACATATACCAAGGTGGTCTCAGAAGG
CTATAACAGAGCTGAAAAATTCCTCTCCCCTAGTGATTTGTGTTACAGTGGCCTACAGTACAGGAATGTGCCATACAGGCTTGTAGC
CTAGGAGCGATAGGCTATACCATACAGCCTAGCTGTGTAGTAGGCTGTCCCATCTAGGTTTGTTTAAGTACACTCTGTGATGTTCAC
ACACTGAAGAAATTGTCCAAGATGTGTGTATTTAAATCACACCCACCGGCCAGGCCTCCCTTTCCCCGTCTCCAACCTCCAAGTAGA
ATACTTCCCTGGCAAGGGTTCCTGGCAGCATAATGGGCATGCTAGTGATTTCTTCTCTGGGGGGCTCTGCTAATGCGACCCCTGGAA
TTTCCCAGGGCTCCTGGGGCTGCTGGAGGCTTAGGGTGCCCAAGAAAGGTGCTCCTTTCATCCCCCTTGTACTCCTGCCCCAAGCAT
TCTTGCCGCAACCGACAGCCCCTCTGTAATAGCAAGTAGCAGGTTACCTGGGCAGAGAGGTGGCCAGAGCAAGTCATTGACATGGGA
TGCTGCGGCCAAGAGCTCAAGGAAGAGGTGTGTGTTCTGTTGTATGTTTTCTTGCTGGCTGGGGAAGTTAGAGGGAAGGAAGCTGAC
ATTTATTGGAAGGAGAGAATATTAACAAAGGTACCATGTATTGAGGCATATGGCCAGGATCTGTGCTAATGTTTTAAGTATATTCTC
ATTTAATCAACACAGTAATCCCATTTCACAGATGCAGGTAGTACTTGCATCACTCTCTAGCCCTGGACCCTGCTTTGTTGTTGTGGT
TTTTTAAGCGCTTGTTACCTGATGCTATCATACAAACTGCATACCGTCTTCTCCGCAGGATTGTAAGTTCCGTAAGCGCAGCAATGT
TATTGATTTGTTCACTGTGGTACCGTCACTTGCTACAACAATGTCTGGCCCATCATAGGTGCTCATTAATTGTTCCTCAAATGTATG
AACAAAGAGAGTCTCAGAGAGTTCAGCAGTGGGTCCAAGGTGGAGCTGATGATCACGTCTATTTATCAAGTTTTTCTGAGGGTTAAT
GAACCAGTGCAGACATATGTGCTTAGCACAAAGCCTGGCACGTAGTAAGCACTCAGTATATGTTAGCTGTCACCATAATTTGCAAGG
TCTTCATTGGACCACTTTCTTGCCCAGGCCCATTAATCTTAGAGCCTTTCCTAAAGTAGATCATAGAGCCCATTGTCTGGCCCCAAT
CAGAGTGAAAGCAATTTTTTGGTAGAGAGTTTTTGGGCTTGCAGAAAACAATCAGCCAATTTGTTTATGGGTTTTGGAGGCAGGACA
GCTGGACTTCCATTCACAAAGGGTCCTCTCCTAACCATCCCCAAACCCATCCGATTGTGGCCAACCTTGAGCCACTCTTTCTGGCTT
GCTGCTTTCATCTTAAGCCCTGCCCTCTCGCACTGCTGCACCCTGAGAGCATCTTTTGGCCCTGGTGCCTCTGCCCACCAGGAGAGG
TCAGGTGGTACCCAAGCCTCCCTCAAGGAAGGGTGCATGGAGCCCTTTGGAGGCTCGGCAAATCTGAGGAAGGGCACATGACCTCAT
GTGCTCCCGACCCCAACAATGCTTAGATCAATCAGCCAGGTGAACTGGAGGGAGACCCTGGCCACAATAGCATTTACTGTCCACCTG
GGAAAGCACAGTGCAGTTAAAAGCCTATTTCTTTTTCTCTCGCAGATATTGATCTACTTAGCTTTGTGCCAGGCTGTTTGCTGGTAA
TTAATTTCAGAGTCAGTGCAAGGGCTGGCAGTTAACATCACTTTCCCGAATGCACGGAGGCCTCCCTCCAAACTCATAAAGATGATG
TGACTGCCTCTCTCAGCTACCCGGGCCGTGCTGTGACTTCAGTATCTTACATTTGGGCTGACGTTAAGTGCAGGACCTGGACTCCGT
GTTCTGTCACATTGCTAAGTGAGGGCCATGATGATTTAAAAAAAAAAAAAATCCAGTGCTCTTTCCAGTTCCTTGATCCTGGAAAAC
AGGAAATGATTTTGTTTGTCTTTCCTTCAGTCCACATGAAAACCTTCTGCTCTTCCTGGACGTGGCTCTTGTTTTGACTTTGCCATT
AGCTAGCTGTGTGATTCTGGCTCTCACATCCCATCTCTGAAGTGGTTTCCTCGTATGTTCAATGAGGGGTTTGAGCTAGATAATATT
AACTAATATCTGTATGGTACTTTACATTTTATGTAGCATTCACACCCACACTGACTCAGTTTGCCATTAATAATTGTCTAGTCTTGG
TCTGTGTCTTGGTTTCTTCATCTGTAAAATGTGGATAATGTTAGCACCTCCCTGATGGGGTTGTGGTGAGGATTAAAGGAGCTTAGA
CATATGAAGTACTCAGACCTGTGGCTGCACCTGGTAGATGTTAAGTGTTGCCCCCAGCACCATCATCACTGCCTCATAGCAGCCCTC
TTTATTTTTCCCATCTTACAGATAATGAAGCATTTGCTCCTAAACTGCTTAATAATTGACAGAGCAGGGATTCACCTGCAGACTTTC
CAGTTTCCTTGAGGCTTTCCTAAGAGGCTAAGTGCGTAGGGAAACTGAAGAAATGACACATCCAATTGCCTCCTCTCCCTGCAAGAG
GAGGGCAGTAGTATTAAATGTGTGCTCTGCAGAGAGCAAGGCTTCCCTTCCGTGCCGCCTTTTTTGCCAGGAGGCTGCTGTTCACTC
TAGTTCCCTGAGCAGAAGCCCAGAGACCACAGTGTCCTCTCCTGGACATCCACCGTTTCCAACTTTCCACCACAACCTGCTTCCTTT
CAGGCAAGAAAGTACTAGAGGTGGGCAGTTAAGTCATTTAAAACTATTTTCACAGCAGATCTTTACTGAATTTTTAAAAAGCAATAT
TTATGTCCCTTAGACATTTCTGAGAAGACTTAGCTGTTCTTTTAAAAACATCACTTCAGCGCCAATGAAAAATATAATCCAGCCTTA
GTAATAAATTTTTAAAACATACACACACAAAAGTTATCAGCCTGCAAAACAATGTTTCCTAGGCTTCCCACACATCTCAGTATTTTA
ACAACATCCCATCTGTTGATGTCAGTCTGAAGAAGCAGGTCTATATTCTCTAATTTTATTAAAATCCTTTCTGGCTAGTAACCTGAT
TTCTATTAGGCTGGCCTACTTATTTCCAGGCCCCAGGCCTGTGGACACATGCCAGTCTGCATGGCTGACAGAAATGGGGATGGTTAA
TCAAAAATCTGCTTAGCATGTGCTTAATAAGGAATGTTTTCTGTTGCTCTTGTTACTAATTGCTGAAGATGTCTGTGTTTGGATTAA
AACTTTGTCCCCTGCCATCTGGATGCCATTGATATGTCTCAGCTCAGTCATGAGCCTGGTGATATCAGTGGTTTCCATGTCAATGGA
TGACAGCGTCATATGTTGAAACGTGTGATTGCAGTGCACAGTGAAGGAGCCAGCCCTGGGGAAGAGGAAGCTCTGGTTTCACACCTA
AATATCTTCACTGATTTTCAGACATTGCTCTGAAATTATTTCTTTTTTGTGCCAATTACATTTACATGTACAATTGCAGTGCAAATG
TTTTTGGCCATTTGGGAAAGGAGACAATGGGCTCGTTGGTACCTATAGTTCCAATCAGTGGGAAAGACATTTTCTCAAAGAAGTCTT
TTAAAATCTCCAGCTGATAGGAAATCTGTTTACTCTCTGAACCCAAGCAATGTACCGTATACACGAAGTGGCAATCTCAGGCAAAGC
TCGGAAGTCTGCATGTAAGGGCAGCAATGGTATATCGCTAGCTATTTTCCAAGGGGACCCCGGGACTCAAGGTCTCGCTCATGCAGC
CTGTCTAAATATAGCATCAGCAACAGCAGTCAAGAGATAGTATTGAAATAATCAAGAAGGAAGAAAGAAAGAAGGAAGGAAGGAAGG
AGAAAGAAAGAGAGAAAGAAAGAAAAAGAAAGAAAGAAAGGAAGAAAGAAAGAGAAAGAAAGAAAGAAAGACCCTCCAACTTCAAGC
TCTGGTCTGCAGTGTCCAACACAAGATTAAGAACAGATGAATTTTAATCACCTTTTTTTTTCCTTTTTTTTTTTTTTTTTGAGATGG
AGTTTCACTCTTGTTGCCAAGGCTGGAGTATGGCGTGATCTCAGCTCACAGCAACCTCTGCCTCCCAGGTTCAAGCGATTCTCTTGC
CTCAGTCTCCAAAGTAGCTGGTACTACAGGCATGTGCCACCATACCTGGCTAAGTTTGTATTTTTTGTAGAGACGGGTTTCACCATG
TTGTCCAGGCTGGTCTCGAACTCCTGACCTCAGGTGATCCACCCGCCTCGGCCTCCCAAAGTTCTGAGATTACAGGCGGGAGCCACC
GTGCCCGGCCTTAATCATCTTTTCTAGGAGTTCCGTGATTTTCGTTAAGAAAATAGATCATTGCAAAATCGTTTCCTTATGAGGTAG
ACTACAGTAGCAACCACAAAAATGAACCTAAGAAAATGAACCTGATAGCAGCCTACTTTATTTATTCTTCCTTAGATCAAGAATGCT
GCAGCCAATGTCCTTCGGGAAACATGGTTAATCTATAAACACACAAAGCTGCTAAAGAAGATTGACCATGCCAAAGTGAGGAAACAC
CAGAGGAAGTTCCTCCAAGCTATCCACCAGTGAGTATGCCAGAGCGGTTCAGCCATCAGATGACCAGACTGGGAGTGGCGGGACTCC
TACAACTCTGTCAGTAGCACAGAAGAAATCCATTCTGTTCCACTCAACAAGTGGAGTGGTGAACTGGGTCTGATCACTCACTGACTG
CACCAGCCACATGCCAGGAATTGGGCAGGCTGCTATGGAAGGTAAAAACTAGAGTAACAACCTCTAAGAGCTTTCACATTCTACACA
CACACACACACACACACACACACACACACACACACACCATGCATTGCAACTCAAATCAAAACAAACCGTGGCTGATGCTTTCTGTGA
TGGAGGAGTTTTTAAAAGTATAGTGGAAAAGCAAAGGATGAAAACATTCATTCTGATGGAGAGGTTTGGGGAATCCCTGAAAAGTTG
AGAATAAGGCACTCAACATGGAAGGTGCAGCCCAGGCATAGAGGCAGGGAAATGATCTGGGAATAGCAAGAAACCAGTGACTGAAGG
TGAGCATGCGTGGAGACAAGAGTTTGCTTTCTGGGGGGATGTGACAAGCCCCTGGAAAGTAAAAGCCTCAGTTGTTCCCCAGGTTCA
CGGTCCCCATGCTGGGTTCCAAAATCAGAGGCAGTGGTAGGGCTGGAGGATCTGCTGGTGGGGCTAAACCTTTGCATGGACTTGAAG
GGCTTGCCTGTTACCATTCTTCCCCACAGGTTGAGGAGCGTCAAGATGGAACAGAGGAAGCTGAGTGACCAAGCCAACACTCTGGTG
GACCTTTCCAAGGTGAGTGGCAGTGTCTGGAGACATCCCCTAGAACACGCAGACACTGAGTCTTCTCAGGCTCACCTGTGAGTCCTG
GCTGGGTTCGCACTGGACAGCGCGGGACCAGGCAAGCAAAGAAAAATTTCTGCTTGGTTCAAAAATACCTAGGCTCAAGTTTGTTTC
ATTTGCAATCTACCGGTATGTCTTGTGGGCTTTTGAAACTTGGTGTTTGTTTCTTAAGTGAAATCGCTCAGGTGTGTCTCTGCGTGA
AATTAATGAGCTGACTAAACCTTTTCCGTGTCATTTTATGAGCTGCACATTGAGGGACTCCTGAGGTTGGGCTTGAGTTTGCACGGC
TGTGAAGCATGCTAGCAAGGACCAGACGGTAACGATGGCCATGGTAACTGCCCAGGTCACACCGGTTTGCTGTAATACTTGGAAGTT
CATTTTTGTACATCCTCCAGTCCTTAACCAACTAGTTTTGCTGGCCTCAAATTGGCGGCAGTGGTGTGTGTGGGGGGGTGCATTTCT
TTACTTCTCAAGAGGCAGATAGCCTACAAGGAACATGAAGATACCCAAAAAGAGAGCCCTCTTTTGAGAGCCCTAGGGTGTAAGGCT
CGCCATCACTCTGCCACTCTGTTTCTGGCCCTGAGCTGCCATAAATACTGTAAGGAAACTCCTCTTGCAAATCACCCAGGTGAGGGA
GCCGAAGGCCACACTGCTGGCAGAGCTGAGAGTCGGTGCCACTGAAGAAAGGGCCTCCCCCTCCTCAGCCCCCTCTCCTCATTGCCT
CTTGATAGGAATTGTATATTAAGTGTACAGCCCAGGGCCAGGCTGGCAGAACATTCCAGCAGATGTCAATTATCATAATGGAACACA
TTCCCTGGGTAAGCAAGAGAAACTCCCTCAGGCACATTAAGCACCCTTTGAAAAGAGTGCAATCAGAGAAATCACTAGTTGTCGGGC
CACGTGCAGGTGCCTTGTTTAATAAAGGGATTGAACCAAAGAAAGGGCCTTCTAAACCTGTGAGGACTTCAATCGGGGAGTAAGTGG
CTTGGCACATTTGGGGAGAAGCCTCCAGGCCCGCGTGCCTTTGGAGGAGGGCGTTCACGTGGAGTGCAGGGTGGTTCCTAGGAGCCT
GGGGTGGGAGTGGAACGTGGGCATGCAGGAGAGAACTCCCTCAGGTTTTTGGGTGTGTTGGTTTTAATATCAGAAACCCGCTGGGAC
TAGGTTTAAAAATGGAGAAATCTATTCTAAGGATATAAGGGAGTCTCATTCCAGGCAAAACCTCAAAGGAGACATGTGGCCAGACTG
CAGGAAGGAACTGACTCGAACTAGACCTGGGAAGCCCCCACAACCAGGAGGTCTCTCACCATTTCTTCTCTGTCCTTCTGTTTAAGT
GACTGCTTCAGTTCTCTCTCCCTCTCCCTGTCTCTCTCTTCCTCTCCCTTTCCCTGTCTCTCTCTTCCTCTCCGTCTCCCTGTCTCT
CTTCCTCTCCGTCTCCCTGTCTCTCTCTTCCTCTCCCTTTCCCTGTCTCTCTCTTCCTCTCCCTTTCCCTGTCTCTCTCTTCCTCTC
CCTCTCTCTTCCTCTCTTTCTCTCCCTCTCTGCCTCCTGTCCCCGCGACAGACTGTAACAGGCTCCAATTCAGAGAGTACATATAAT
AGTTTTAGCCACAGGTAAAGACTAATTGGCCCCCTACTAGTCTTAATTCTAAATTCTCAGAATAGAAGAAATGATTGGCCAATTTGG
GCCACGTATCCACCCTGGGACCAATCAGCATTGGCCAGGAAGAGGGTCATATGGAGCAAACAGGGCTAGGGGCCCACCTGTGAGCTT
CAGGAATAGCCCAACAAACTGCCCCAGGATGTTTCTTCCATGAGTAGCAGTATTTCCTGTTTCTCCTCTCACCTTGCAGGACATGCT
GTGACTGATGTTCCCTGGGGACATGAGGCAAACCAAACTTTCTTTTTTTTTTTTTTAGTTTTATTATTATTACACTTTAAGTTTTAG
GGTACATGTGCACAATGTGCAGGTTTGTTACATATGTATACATGTGCCATGTTGGTGTGCTGCACCCATTAACTCGTCATTTAACAT
TAGGTATATCTCCTAATGCTATCCCTCCCCCCTCCCCCCACCCCACAACAGTCCCTGGAGTGTGATGTTCCCCTTCCTGTGTCCATG
TGTTCTCATTGTTCAATTCCCACCTATGAGTGAGAACATGCGGTGTTTGGTTTTTTGTCCTTGCGATAGTTTGCTGAGAATGATGGT
TTCCAGTTTCATCCATGTCCCTACAAAGGACATGAACTCATCATTTTTTATGGCTGCATAGTATTCCATGGTGTATATGTGCCACAT
TTTCTTAATCCAGTCTATCATTGTCAGACATTTGGGTTGGTTCCAAGTCTTTGCTATTGTGAATAGTGCCACAATAAACATACGTGT
GCATGTGTCTTTACAGCAGCATGATTTATAATCCTCTGGGTATATACCCAGTAATGGGATGGCTGGGTCAAATGGTGTTTCTAGTTC
TAGATCCCTGAGGAATCTCCACACTGACTTCCACAATGGTTGAACTAGTTTACAGTCCCACCAACAGTGTAAAAGTGTTCCTATTTC
TCCACATCCTCTCCAGCACCTGTTGTTTCCTGACTTTTTAATGATTGCCATTCTAACTGGTATGAGATGGTATCTCATTGTGGTTTT
GATTTGCATTTCTCTGATGGCCAGTGATCATGAGCATTTTTTCATGTGTTTTTTGGCTGCATAAATGTCTTCTTTTGAGAAGTGTCT
GTTCATATCCTTCGCCCACTTTTGGGCAAACCAAACTTTCAAGCCTGCATTTCTGCAGTGCCACCCACGGCCTCTTGGAGAGATGGG
GCCCACGGAGCAGACCGGGTGAAGCGAACCCCTCAGTGAACTGGAAAGACACTCCTGTGAATTGGGTGGGGGAACTAAGCCGTAAAT
AACCCAGGAGCAAGTTACCTGGAAAGAAAGAGTGTGCAGGAAAGGGTTAAGGGAAAAATCTGCAGAACCCCCGTGGGGACATCTGGG
GAGGAGATGAACTCTGACATTCGCCCAGTCTTTGTCTTTTCCTCCCAGGAGACTCACCCAAGGTCCTGGCAGCAGCTTAGCACACTT
CACACTGGTCCATATTCCCCCTGGCATCCTGTCACCAAGCAACGGAACAGGGGGAGACAATAACGCTGTGTTCTCTCTCTTTTCTGT
CTTTGTGCTGTAGCCTCCCAAGTCAGCGCCTTCCCACTCCTGCCCTTTGGTCCTCTCTAAATTAAGAATATGGCATGTGGCCTCCAC
ATATTACGTCTGTATTGAATTGCTGTCCTATAGGCTGGCAGAGCTGGGAGGGCTTGGAGTCTTTCCCCGATTGCTTCAATTACTTCA
ATTCAAATATAAAAATAATTCCTACCATCGGAGGCTCAGGTGATCGAATAAGATGATATCTGTGAAAATGCTTTGTAAACTATAAAG
GTTTTGCAATTGGTAGTTCTGATTATCAATATAGATGAGGAAACCAAGATCCAAGGGAACAAAGTGACCATCCCACGGTCAGGCAGT
TGGTGAGCACCAGCGTGGGGGCCACAACCCAGGGGCCTGGACTCTGAGTCCAGAGCTCATCAGGGTGCCCCTGCGGTCAAATGAAAT
GGGTGATTTTGCACATTCCGGGAAGAATGTTTGCCTCGAGGAGGTTCTGTCATGTTGATTAATAAAGGGAGTACTTTCACTTCTAAA
GTCACCCTGTCCTCCAAGGGCATCCACGACACGGTGTCCTGCTCTGGCAACAGCAGAGCTCATGGGAGCGGGGCCATCTCCATGCCA
CCTTGTGACCTTTCCTTATGCTGCCCCAGTGAATTTCCTCATGGGGCCTGTAGTCTGCTGGGGAGGCCTCACTGCATTTTGTGCCCC
TGCTCCCCTTCATTCTTCTCTGGCTTCTCACATCTTCTTTTACTCTTCTTTTTTCCTCTCACCTTACTTGCTGCTGCTTTGGTCCCT
GGGGCATAAATCATGTCCATATCTTCCTAGAGTAACCTGAAATTTCACGTGAAATAGGATCGGGGTGTCACTGGCAGATCATCAGAA
ACCCAAAGAGCCACAGTACCTCAGGGATGACCAAGCAACCACCGGCCTGGCCCTCTGCATTGCCGCCCCAGGAGCCTCACTCCCTGA
CACGTGGGAGGCTGAAGATGCTTGTGAATCTTGTTCCCAAGTCTGATGTACACAGCCAAGATGGCATTTGCTAATTTGCTGGGAGAA
AAATATCTGTTGCATAGACTGGGACTCTGCAGACCAACAAGGGGGCCACAGGATGTTGGGGAAGGAGCACTGGTAATCGGCCGGCAC
CCCTGGAACACGGGGAGTCAGTGCTGAGTGCAGGAGGGGTACTGGGGAGCTCTCAGGGGAGCACCCTCAGGACCCCTGCCAAGGAGT
GAGGGAAGCAGGATTGGTCAGTGGGAGGGTTTGAACTGTGATACAGTCAACAAAGACCTCAGCCCTTCAGAGTGGCCCTTCAGGGAT
GTCCCACCCTGAGGCATTGTGGATCAGGCAGGCGAAGGGCCACACCTCAGCAAGGCAGCCTCCTTTGGCTAAGCACTGTGCCTGAGA
GTTAATTCACTTTACATCCTGTCTGCTGGGTTAAAATAGAGGTTGGGAGATGTGCAATAACTCCTAACTGTGTACAGTTATTATTAA
CTCCAATTTTATAAGGCAACCGAGATTGAGAGAGGCCAGGCGCCTGGCAGAGGGCACACTGTTAGTGAGCTGCAGATGTGTACTTGA
GCCCTGGCCTACTAATTATACACCCACTGCACTGTCCATTCCCCACCAGCGTCTTCTCTGGAATCATTAGATGCCAGCAGCCATGTG
AGCTGAGAGGTTGTGGCCCTGTGAGAACATGAGACAGTTTAGAGACTCACTTTATTTAAGAAAAAAAAAAGGGCCATTTTTTTCCCC
TTGGTGCATATTTTCTAATAAAATGCTGTCATTAATACTCATTACTTTTGTAATAAGAAAACAAAAATGTGCACACTTCAGCATCCC
CATCCCCAGGAAAGAAGCCTGTATCCTTCCTTGACCAGATCAGCTGGTGTGGAAGTGGCTCCTGTCATACTGTGGAGGGCGTTTCAT
TTCTCCCATTGCAGATTCCTCTGTGATGACCTGTCATCTCCCTGTCTCTCGCCTCTCTCTCTCCACACAGATGCAGAATGTCATGTA
TGACTTAATCACAGAACTCAATGACCGGAGCGAAGACCTGGAGAAGCAGATTGGCAGCCTGGAGTCGAAGCTGGAGCATCTCACCGC
CAGCTTCAACTCCCTGCCGCTGCTCATCGCCGACACCCTGCGCCAGCAGCAGCAGCAGCTCCTGTCTGCCATCATCGAGGCCCGGGG
TGTCAGCGTGGCAGTGGGCACCACCCACACCCCAATCTCCGATAGCCCCATTCGGGTCAGCTCCACCTCCTTCCCGACCCCGTACAC
AAGTTCAAGCAGTTGCTAAATAAATCTCCCCACTCCAGAAGCATTACCCATAGGTCTTAAGATGCAAATCAACTCTCTCCTGGTCGC
TTTGCCATCAAGAAACATTCAGACCAGGGAACGGAAAGAAGAGAGACCGAGCTAATTAACTAACTCATGTTCATTCAGCGTGCTTGG
TCCGACATGCCTTGAAACCAGAAATCTAATCTCTGTTTAGGTGCCTCTACTTGGGAGCGGGAAGAGGAGATGACAGGAAGCGACGCC
TCTGGCAGGGCCCTTGCTGCAGAGTTGGTGGAGAACAGAAATCCACGCTCAATCTCAGGTCTTCACGCGGGGGGTGGGGGTCAGATG
CACTGAAGTAGCCAACAGCGAAGCCAGTCCAGAAGAGGGGTCCGCTGGGAGGGAGGGTTGTGTCAGGCTTGGGGGATGGGCTCTTCG
CCATGGGGGTCTTTGAACACACCTCTCTCCTTTCCTTTTGTCTACGGAAGCCTCTGGGTGACAAAAGTAAAAGAGAGCTGCCCACAA
CTTGCCAAAACAGATATACTCGACTCAGACTGAAAAAAAAAAAAAAA
HUMAN mRNA SEQUENCE:hR27-009.1 (Seq ID NO: 27)
CTTACAATGTAACAGTGGCAGGAGGAGGCGAGCGAAGCTATTGAGCCAGCGAGGAGTGAAGCTGAGCCTGGCCTCACACGCTCCTAG
AGGACCACCTCCTGAGAGAGTTCTTTCACCCCCTCTTCTTTCTCCAAGCTCCCCTCCTGCTCTCCCTCCCTGCCCAATACAATGCAT
TCTTGAGTGGCAGCGTCTGGACTCCAGGCAGCCCCAGAGAACCGAAGCAAGCCAAAGAGAGGACTGGAGCCAAGATACTGGTGGGGG
AGATTGGATGCCTGGCTTTCTTTGAGGACATCTTTGGAGCGAGGGTGGCTTTGGGGTGGGGGCTTGTGCTGCAGGGAATACAGCCAG
GCCCCAAGATGGACACTTCTGGGCACTTCCATGACTCGGGGGTGGGGGACTTGGATGAAGACCCCAAGTGCCCCTGTCCATCCTCTG
GGGATGAGCAGCAGCAGCAGCAGCAGCAGCAACAGCAGCAGCAGCCACCACCGCCAGCGCCACCAGCAGCCCCCCAGCAGCCCCTGG
GACCCTCGCTGCAGCCTCAGCCTCCGCAGCTTCAGCAGCAGCAGCAGCAGCAGCAGCAGCAGCAGCAGCAGCAGCAGCAGCAGCAGC
CACCGCATCCCCTGTCTCAGCTCGCCCAACTCCAGAGCCAGCCCGTCCACCCTGGCCTGCTGCACTCCTCTCCCACCGCTTTCAGGG
CCCCCCCTTCGTCCAACTCCACCGCCATCCTCCACCCTTCCTCCAGGCAAGGCAGCCAGCTCAATCTCAATGACCACTTGCTTGGCC
ACTCTCCAAGTTCCACAGCTACAAGTGGGCCTGGCGGAGGCAGCCGGCACCGACAGGCCAGCCCCCTGGTGCACCGGCGGGACAGCA
ACCCCTTCACGGAGATCGCCATGAGCTCCTGCAAGTATAGCGGTGGGGTCATGAAGCCCCTCAGCCGCCTCAGCGCCTCCCGGAGGA
ACCTCATCGAGGCCGAGACTGAGGGCCAACCCCTCCAGCTTTTCAGCCCTAGCAACCCCCCGGAGATCGTCATCTCCTCCCGGGAGG
ACAACCATGCCCACCAGACCCTGCTCCATCACCCTAATGCCACCCACAACCACCAGCATGCCGGCACCACCGCCAGCAGCACCACCT
TCCCCAAAGCCAACAAGCGGAAAAACCAAAACATTGGCTATAAGCTGGGACACAGCAGGGCCCTGTTTGAAAAGAGAAAGCGACTGA
GTGACTATGCTCTGATTTTTGGGATGTTTGGAATTGTTGTTATGGTGATAGAGACCGAGCTCTCTTGGGGTTTGTACTCAAAGGTAG
GGGCTGTGGTTTCTCTTTATACCTTGAACAAAAGGAATATGTAGGTAGCAAGAGAGGGATTGAGAGAGGGGGATTGTGAGAGAGAGA
GATTGAGAGAGAGAGAGAGAGAGAGAGAGAGAGAGGAGACTGGGAGAGAGAGGTGGTGGTGGTGGTGAGAGGCGCTTGCTCAGTTAT
ATTCAACACTCTAACTTCCCGTGGTTTTCCTTCTTGATCCCGGGAGAATTCATTCCTAGCTTCCTCTGGGGGGTAGGGGACATCCCC
ACACACTGTGTCTAATTTGCAGACTCTCTGTTAGGGAGGATTCAATAATCCCTTCTGAGAAATCGCTTTTCCTCCTGGCTCACTCGT
ACTAAAGCATAACCCAGCAGCTTAACGCACCAATTAATTACACTCCGCCTTCATGTGTTTGCCACTTCCTGCGACTTCCCCTTCTTT
CCTCCCCTCCCTGCTCCACTCCATTTTCCTGAGGATAAAAAAGAAATAAATGTCTGGGTGGGGGGTGGGGATTCCGTACCATTGCCC
AAGTCCCTTCTCTCCCTGTCTTCTCCATCTCTTCCCAAGTTTCTGTTTCCCTCCTCTTCTCAGAGAGTCTCGGGCAAAATAGAAAAC
CATCCATGACCTGGGTCTCCTGAGGGCATGGGGTAAAAATATAGGTGGAGGAAATCAGTGGTCCTTTCCCTCAGGAGAGAGGACTCA
GAATAAACCTGCTGCTGCTTCGT
HUMAN PROTEIN SEQUENCE:hP27-009.1 (Seq ID No: 28)
MDTSGHFHDSGVGDLDEDPKCPCPSSGDEQQQQQQQQQQQQPPPPAPPAAPQQPLGPSLQPQPPQLQQQQQQQQQQQQQQQQQQPPH
PLSQLAQLQSQPVHPGLLHSSPTAFRAPPSSNSTAILHPSSRQGSQLNLNDHLLGHSPSSTATSGPGGGSRHRQASPLVHRRDSNPF
TEIAMSSCKYSGGVMKPLSRLSASRRNLIEAETEGQPLQLFSPSNPPEIVISSREDNHAHQTLLHHPNATHNHQHAGTTASSTTFPK
ANKRKNQNIGYKLGHRRALFEKRKRLSDYALIFGMFGIVVMVIETELSWGLYSKVGAVVSLYTLNKRNM*
HUMAN mRNA SEQUENCE:hR27-009.2 (Seq ID No: 29)
GCCTCACACGCTCCTAGAGGACCACCTCCTGAGAGAGTTCTTTCACCCCCTCTTCTTTCTCCAAGCTCCCCTCCTGCTCTCCCTCCC
TGCCCAATACAATGCATTCTTGAGTGGCAGCGTCTGGACTCCAGGCAGCCCCAGAGAACCGAAGCAAGCCAAAGAGAGGACTGGAGC
CAAGATACTGGTGGGGGAGATTGGATGCCTGGCTTTCTTTGAGGACATCTTTGGAGCGAGGGTGGCTTTGGGGTGGGGGCTTGTGCT
GCAGCGAATACAGCCAGGCCCCAAGATGGACACTTCTGGGCACTTCCATGACTCGGGGGTGGGGGACTTGGATGAAGACCCCAAGTG
CCCCTGTCCATCCTCTGGGGATGAGCAGCAGCAGCAGCAGCAGCAGCAACAGCAGCAGCAGCCACCACCGCCAGCGCCACCAGCAGC
CCCCCAGCAGCCCCTGGGACCCTCGCTGCAGCCTCAGCCTCCGCAGCTTCAGCAGCAGCAGCAGCAGCAGCAGCAGCAGCAGCAGCA
GCAGCAGCAGCAGCAGCCACCGCATCCCCTGTCTCAGCTCGCCCAACTCCAGAGCCAGCCCGTCCACCCTGGCCTGCTGCACTCCTC
TCCCACCGCTTTCAGGGCCCCCCCTTCGTCCAACTCCACCGCCATCCTCCACCCTTCCTCCAGGCAAGGCAGCCAGCTCAATCTCAA
TGACCACTTGCTTGGCCACTCTCCAAGTTCCACAGCTACAAGTGGGCCTGGCGGAGGCAGCCGGCACCGACAGGCCAGCCCCCTGGT
GCACCGGCGGGACAGCAACCCCTTCACGGAGATCGCCATGAGCTCCTGCAAGTATAGCGGTGGGGTCATGAAGCCCCTCAGCCGCCT
CAGCGCCTCCCGGAGGAACCTCATCGAGGCCGAGACTGAGGGCCAACCCCTCCAGCTTTTCAGCCCTAGCAACCCCCCGGAGATCGT
CATCTCCTCCCGGGAGGACAACCATGCCCACCAGACCCTGCTCCATCACCCTAATGCCACCCACAACCACCAGCATGCCGGCACCAC
CGCCAGCAGCACCACCTTCCCCAAAGCCAACAAGCGGAAAAACCAAAACATTGGCTATAAGCTGGGACACAGGAGGGCCCTGTTTGA
AAAGAGAAAGCGACTGAGTGACTATGCTCTGATTTTTGGGATGTTTGGAATTGTTGTTATGGTGATAGAGACCGAGCTCTCTTGGGG
TTTGTACTCAAAGGACTCCATGTTTTCGTTGGCCCTGAAATGCCTTATCAGTCTGTCCACCATCATCCTTTTGGGCTTGATCATCGC
CTACCACACACGTGAAGTCCAGCTCTTCGTGATCGACAACGGCGCGGATGACTGGCGGATAGCCATGACCTACGAGCGCATCCTCTA
CATCAGCCTGGAGATGCTGGTGTGCGCCATCCACCCCATTCCTGGCGAGTACAAGTTCTTCTGGACGGCACGCCTGGCCTTCTCCTA
CACACCCTCCCGGGCGGAGGCCGATGTGGACATCATCCTGTCTATCCCCATGTTCCTGCGCCTGTACCTGATCGCCCGAGTCATGCT
GCTGCACAGCAAGCTCTTCACCGATGCCTCGTCCCGCAGCATCGGGGCCCTCAACAAGATCAACTTCAACACCCGCTTTGTCATGAA
GACGCTCATGACCATCTGCCCTGGCACTGTGCTGCTCGTGTTCAGCATCTCTCTGTGGATCATTGCTGCCTGGACCGTCCGTGTCTG
TGAAAGGTACCATGACCAGCAGGACGTAACTAGTAACTTTCTGGGTGCCATGTGGCTCATCTCCATCACATTCCTTTCCATTGGTTA
TGGGGACATGGTGCCCCACACATACTGTGGGAAAGGTGTCTGTCTCCTCACTGGCATCATGGGTGCAGGCTGCACTGCCCTTGTGGT
GGCCGTGGTGGCCCGAAAGCTGGAACTCACCAAAGCGGAGAAGCACGTTCATAACTTCATGATGGACACTCAGCTCACCAAGCGGAT
CAAGAATGCTGCAGCCAATGTCCTTCGGGAAACATGGTTAATCTATAAACACACAAAGCTGCTAAAGAAGATTGACCATGCCAAAGT
GAGGAAACACCAGAGGAAGTTCCTCCAAGCTATCCACCAGTTGAGGAGCGTCAAGATGGAACAGAGGAAGCTGAGTGACCAAGCCAA
CACTCTGGTGGACCTTTCCAAGATGCAGAATGTCATGTATGACTTAATCACAGAACTCAATGACCGGAGCGAAGACCTGGAGAAGCA
GATTGGCAGCCTGGAGTCGAAGCTGGAGCATCTCACCGCCAGCTTCAACTCCCTGCCGCTGCTCATCGCCGACACCCTGCGCCAGCA
GCAGCAGCAGCTCCTGTCTGCCATCATCGAGGCCCGGGGTGTCAGCGTGGCAGTGGGCACCACCCACACCCCAATCTCCGATACCCC
CATTGGGGTCAGCTCCACCTCCTTCCCGACCCCGTACACAAGTTCAAGCAGTTGCTAAATAAATCTCCCCACTCCAGAAGCATTACC
CATAGGTCTTAAGATGCAAATCAACTCTCTCCTGGTCGCTTTGCCATCAAGAAACATTCAGACCAGGGAACGGAAAGAAGAGAGACC
GAGCTAATTAACTAACTCATGTTCATTCAGCGTGCTTGGTCCGACATGCCTTGAAACCAGAAATCTAATCTCTGTTTAGGTGCCTCT
ACTTGGGAGCGGGAAGAGGAGATGACAGGAAGCGACGCCTCTGGCAGGGCCCTTGCTGCAGAGTTGGTGGAGAACAGAAATCCACGC
TCAATCTCAGGTCTTCACGCGGGGGGTGGGGGTCAGATGCACTGAAGTAGCCAACAGCGAAGCCAGTCCAGAAGAGGGGTCCGCTGG
GAGGGAGGGTTGTGTCAGGCTTGGGGGATGGGCTCTTCGCCATGGGGGTCTTTGAACACACCTCTCTCCTTTCCTTTTGTCTACGGA
AGCCTCTGGGTGACAAAGTAAAGAGAGCTGCCCACAACTTGCCAAACAGATATACTCGAATCAGACTGAAAAAAAAAAAAAAAAAA
HUMAN PROTEIN SEQUENCE:hP27-009.2 (Seq ID No: 30)
                              MDTSGHFHDSGVGDLDEDPKCPCPSSGDEQQQQQQQQQQQQPPPPAPPAAPQQPLGPSLQPQPPQLQQQQQQ
QQQQQQQQQQQQPPHPLSQLAQLQSQPVHPGLLHSSPTAFRAPPSSNSTAILHPSSRQGSQLNLNDHLLGHSPSSTATSGPGGGSRH
RQASPLVHRRDSNPFTEIAMSSCKYSGGVMKPLSRLSASRRNLIEAETEGQPLQLFSPSNPPEIVISSREDNHAHQTLLHHPNATHN
HQHAGTTASSTTFPKANKRKNQNIGYKLGHRRALFEKRKRLSDYALIFGMFGIVVMVIETELSWGLYSKDSMFSLALKCLISLSTII
LLGLIIAYHTREVQLFVIDNGADDWRIANTYERILYISLEMLVCAIHPIPGEYKFFWTARLAFSYTPSRAEADVDIILSIPMFLRLY
LIARVMLLHSKLFTDASSRSIGALNKINFNTRFVMKTLMTICPGTVLLVFSISLWIIAAWTVRVCERYHDQQDVTSNFLGAMWLISI
TFLSIGYGDMVPHTYCGKGVCLLTGIMGAGCTALVVAVVARKLELTKAEKHVHNFMMDTQLTKRIKNAAANVLRETWLIYKHTKLLK
KIDHAKVRKHQRKFLQAIHQLRSVKMEQRKLSDQANTLVDLSKMQNVMYDLITELNDRSEDLEKQIGSLESKLEHLTASFNSLPLLI
ADTLRQQQQQLLSAIIEARGVSVAVGTTHTPISDSPIGVSSTSFPTPYTSSSSC*
```

TABLE 4

MOUSE GENOMIC SEQUENCE: mD27-014 (Seq ID No: 31)
AGCTCGGCCAGGGGAAAGTGAAAGTTTGCCTCGGTGCTCTCGGTGTCGCTGCGGCTCTCTGCATCCCAGGACAGCGGCGTGGCCCTC
GACCGGGGCGCGGGCTCTTCAGCCACTAGCGAGCAAGGGAGCGAGCGAACCAGGGCGGCCAACACGCCGTGCCGGGACCCAGCTGCC
CGTATGACCGCGCGGGGCGCCGCGGGGCGCTGCCCTTCTTCGGTAAGCTGCAACCGTGCCGCGCGGGGCCCGGGCCGGGCTGGGGCA
GGAGCTCTGCAGCAAGCAGCAGGCGGCTCTGCGGGGCCACTTGGAGCGGACAGCCCCTTCTCGCCAGCTGCCCAGGCTTCTCGGCCC
TGGGTTCTGGCTTCCTTACTGCTTCTAAGCTACGGCTGAGGCCCGCGGTATTTCAATAGCTGCTCCTGGGGCTGCAGCGCTTTGCGA
GGTAAAGAGAAGGCTGCTCATCCCATTGCACGGAGGAGAACACTGAGCTACCCACAGGCTGGAGAGAGAGATGGGGGTCTGGGGGGG
GGGGGTATCGCAGGGATTGGCACAGGCCCAATGTCTGCCAGAATGGTCAACCAGGTCTCCAGGTTCCGAGTGACACAAGATCCGAGT
GTGGCATCATCCCCAGTAGATATCTTTCTTTGGTTGTTGCACATGTTTTAGGCCCCAGTTGGGAGTGGGCCTTCAGGGTAGGGAAAA
TTCAGGCTCACTCAGCTTTACACCAGGACCTGTGCTGAGCAACCTGTTGGCAGAGAGAGAGAGAGAGAGAGAGAGAGAGAGAGAGAG
ATGGGCATGCTGCTACTTGTTCAATGGATGTGTAGGAACTATTAGGGACTTGGGTGGCTCTTTGAGGGGAGCTGGACCCGAATCTTT
TCTTGAAGAAAATGAGATGGGGCTATTCTAGACTCTGCGGCTGACTGAGGAAAGAAAGTGCAAGGAGAGTTCAGAGCTGCAGCTGAT
AATGGGCTGCTCAGAAGCTGTCTCCAGTAATGGCAGCAGATGATAGCATGGCTCTTACAGAGTGAAGGATGGTACTTTCAATCCACA
GCAAGTGGGTTTGCTTCAGAAGAGAATGAGAGGGAAAACCTGGAGGGTAGGAACCGAACTTGTGATGGGCAGCGCGTTGCAGGTGTC
TGGGAGGAGAGAAAAGTAGAACAGCAGAGAGAGGGGCTCAGTGAGAGGCAGAAGGTGGGGTTAGGAGGAGGGGAAGGGAGGAAGTTA
TCCCACATGCAACAAGCATAAAGAGAGAAGAGGGAAGAAAAGAAAGACTACAAGGAGACATAAGAGGAAAGAGAGGAGGCCGAGGTT
CAGCCAGGCAGTCAGGCCGGGTCTCTGTCTGCAGACTGTACTGATTGGAAAATGGAACATGATGTCTTGAAGTGCTCAGAGACTGCC
GCAGTTTCCAGGCTGCTCTCATAGTCAGAGGCTGAGGCAGTGGGTGGGAGGAAGAAGCAAGCCAGGAAGAGCCTGTCCAGCCAGATG
CAGGCTAGAGTATTAACCCTTGGGTGTCCATCTGTATCCCTGTCTATGGCAGCATTGGGGTTACAGAGCCAGATCCAGGAGTGGCTG
GTCATCTTTGAAGCCAGCAGCTGTGCATTATTTCTGTGCATGCTTGAAATGCCACCAAAAGCCAGGTAGCCCTGAAGTAATGTCTTT
ATCTTGCTATCTCCAAACATCAATGGCTTGAAATTCCTGGGTTGAGATATTTCTGAACATCTCCATTCCCGGGTCCTTTCCTTCATA
CCATACACACATGGACTGACAGGCTGAGTGATACCAGCCTGGGCCAGAGTCATCTTCTGCAGTCATTTCCTTTAACAGGTTCTGAGA
CTGGGGTTCAGGAGACCCGGGCTGCCACTCCCACTCAGGACTAACTTTCCAATTGCCCAACCATGCCCAATGCAGTTTATCAACCCA
TAGCTGGCTGCACCCTCTCCCCCACCTCTTTCTCCCTGTCTCTCTGTGTCTGTCACTCTGTGTCTCTGACTGTCTGTCTCTGACTGT
CTGTCTCTGACTCTCTGTCTCTCTCTCTCTCTAAGGTCAGGCAAGGAACCTAACAGGTCCTGTGCGTAATGGCATTAGGCAACCCTG
TGCCTTAACACCCATCAGCATCTTCCACCCTTTAAGGCCTCACTCTTCCCTCCGCATGGCCCAGCCAAAGCTGCCTTTCCTCCCTGG
TACTCACTGGCAAGGCTTTGGGCTTTTAGATGTAACCCAGTGTTGTTATGGACTGTTTCATAGCCTCCCTGTGTTTTTCCAATGCGC
TCCTATTTGCAACATTTGTCTCATCATCTGGACTATTGGGGCAGAGCCAGATCCAAGAAGGTGCCAGTCATCACTCCACCCTGCCAG
CATTTCCCTGAATTGGGGGATATCAGCAGCCAGAATGCTCAGAATGGCAGCATCAGGCCAGGGAAAGAAAGCCCAAGAGTCCCTCTG
ACCCCAGGCTGGCATCTCCCGGGATACCCTATTGGAGCCCGAGGGATGGAAGGATTTTAGAGTAGAGGAGGAGGAAGGAAGAAAGGA
GAAAGGAAGGACCCAGTCAGGTGTCAGGAGCCAGACCTAGGACTCTAGGGATGAGCAGCTGCAGGGCCAGGTGGGTGCTGACGGAAA
GGAGAGAACTTTCTTGCTGTGGCAAGAGTTTGCTGACTAGATGAAGGGCCTGATGGGTCTCAGATAAACAGATGCCAGCAGGGAAAC
ACAGCATTGTCCTTGATTAGTCAGCTGGACTCCTCTTGAAATGCAAGTCCAAGGCTCTAGGAGGTCTCCCACTGGAGCTGAGTTCCC
AGTGTTGCATCCTGGTATTTTCCCCTGTAGAACCAGACCTGTGTAACCAGGTGGGAAGGAAGGAATGAAGGAAGGAAGGAAGGTGCC
AGGGGTAATGAGTGTTTCTGGCTTGCAATCTGAAAGCTAGTGACTGACCACAAGTTTGAAGGCAGCTTACAAGGTATCTATTTGTCT
CAGTGTTAAAAGTTAAAAGGATAGAAAATCTTACCCTTGAGTCTCTCAGTCACTAAAACTTATACTGGTTGGCACACCACCAGTATT
ATCATTAGACATTCTCTGTAAGATGGAAAGGTGGCTCATCATTTAAAAACACTTGTTGCTCTTGCAGTGGATCCAGGTTCAAATCCC
AGAATCTAGATGATGGTTCACAACCATTCATACCTCCAGGTCCAGGAGATTCCAAGAAGGAAGCACATAGAGTGTGTGCATGCAAGC
AGGCAAAATATTCATAAACACAAAACAAAAACAGTAAGCCTTTTAAAATTGTCTTTTAAAGTACTCTGTATTGTTGAGCTGCTCTGG
GTTAACCTCAGAAGAGTTCTTGGAGAAGATGGCATTTGGGCTATTACCAAGGGAAGAGTGAGGTGGTGCCCTGGCTGATAAGAATCT
AATGGTGCTGCAGCATGGGAATGGGGCGTTGTTTCTTTGCCCTTAGTGGGACATGGGGGTAGCTAGGGAGAGGAAGTGCTGGAAAGA
CATGACTCCCTTCTGTTATAGACATGGCTGGGCTCCCGGCTGCTGCTGGTCTGTCTCCTCATGAGCAGGAGTATTGCCAAGGAGGTG
TCAGAACACTGTAGCCACATGATTGGGAATGGACACCTGAAGGTCCTGCAGCAGTTGGTGAGTATGAGACCATGCCTTCTCCTCACT
GAGGAAACTGAGGTAGCAAGCCAGAAGGCAGAGTCAAAGAGAGGCTTCCAGAAAACAGTCAAGTGGATAATGGCTCTCCTGGTACTT
CTCTCATTGATCTTTTTAGGTCCTCCCAACCCCTTAGTCTGGGTCACCTGTAGTGACTAGGGTGCGCTTCTAACTGCTGCCTGGGTT
AATGGTTGTCCAGGAATGTCCACTATGGATTCTAAACTGCTCCTTGACCCTGCTCTGACTCAGACAATGCCTCCTTTGGGAAGTCAG
CCCAGTCTGCCTGCTGGGTCCAGATGGTTCATCCTGCTCCTGGATAGCCAGATCCCTGAGCGCCTGGCACCAGGGCCCCACCCAAAT
TTAGATGGGAGGCTCTAGCCTAACTCTGCGCCTTCTGCCTTCATTCCCCTGATCCTTGCCCCTGAGGTTGGAATGCCCTGCTCATCT
GGGCCAGGAAGGAACCCAGGTCCCATTCTAATAGGCTTAATGCAATCTTCAACAATTCTAAATGTCATCTAGCCAGGCAGGATCCAG
TTGTTGTCATGGTTCCTGCTCCTCTCACTAAAGAATTTATTCCACAAGCCCGGCAGGGTGGGGCACCAGTGGGTAAGGCCATGTGCA
GAGCCTGAACTTGGGCCCCCGAGCTGACCTTCCCAATCTCCCTTCTTAACAGCACCCTATGCATGCAGCTTCCATGGCAGGCCCAAG
GCCATGAGTGCTGTCGTGGTCTCTGCTCCAGGGAGAGGCAAGGGCTAGAGACCCAGACTCCCCAGAGCTCAGCCAGCCTCTCCCTGA
GCCTTTTCATGATAGAGTGCTTACCACCTTCCAGAAAAGATTACATCCTATATTTTCCTAGAGATAATTCTTCCATATACAGACTAG
ATTGTGCAATTTCATAGGTCAAAGTAGAAGTGTCTGTTGTTGGCTTTTAAGTACTCTGGGTATTTGGAAGATACAGCTTTTCCCCAG
CTGTGGCTCTGTCATTTTGGCATTGTTTTAGGGAGGTAAAATTAAAACATCATTCACCCCAAAACCTGGGTTCCCCATCCTGAAGCT
GAGTGGACCTGTAGGTACTATGTAACCACTGGCCTGTTCTATCTCACAGATCGACAGTCAAATGGAGACTTCATGCCAGATTGCCTT
TGAATTTGTAGACCAGGAACAGCTGGTGAGTAGCAAGTGTCTGTAAGGTGCTTAAGTGAATATTCACGGCCCGCTCTGTGTTGGCAT
GGAACAGTATGAAGGCAGCAGAAAACCTAGAGCAGACAGGATGAGGGTCCCTGGGTGCTCAAGTGTCAAGTTCCAGCTTGCAGGGCT
GGGTAGGGTCCAGAGCTTAAGTCCTTCTGTTTCCTTACTTTGACCTGGTATAAATCAGGTACTTGCTAAATGCTAGGCACATTCCCC
AATTAATCCTAACAATACAATTCTGAGGTCCACAAATATTGCTTGTTACCCATGCAGAGATTAAGGAGCATGCTTAGCCAAGTGATA
GAGGCCTAATTTGAGCCCAGGAATGTTATACCTTGTAAAGTAAAAAATGTGCTTTATTTTTTAGATCACATCTTCTCCAATCCACTG
GCCAGAGACAGGAAGGCAGTCTAGCCTGGCTTTCTACCCCACACCTAACTGACCAGCCACCCAATGCAGCCAATACTCCCTCTCTGC
ACCTTAGGGTGCTGTCCCTCTCTCCTCCCCAGTCCCTTGGAAATTTTCAGGGGCTTCTTGACTTCCCTGATTCTAGGGCACTCTCCA
TCTTACCACTAGGGGGAGCCCTCTGAAACCCCAAAGCCTTCACTTATTTCGCCTTACTTGTCAGGAGCCCCATGGCTCCAGGAGAAA
GCACAGTCTAGTTCCAAGTCTGCAGTACCTACCACACCCTACGTTCATTTCCCTCCTCTGCTTCATTCTGCTCCCCAGCTTTTGCAT
CAGCTCTTTGTGATGTCTTTCCATCCTTTCCATGTGCCCCGCTGATCCCTGTGCTACACACCTCCCTATCTTCAAGCCTCAAGAGAA
CAGTCAGGCTACCCAGAATTCCTTCTATCTCTGAGCTCTCATGCACTAGACCCATGCATCTCTGTGAACATGCCATGGCCTGCATAG
TCTCAGGGTTTCTTCCATCCATTCTATTGCTCCTTCACTTGCACCATGGCACCTATTTATTGGATCAGGCACTAGAGATACAGAGAT
AAAAGTAGTTTCTGTCTTTGAACCAACTACTGTATCCTTGGAGACAAAACCCAAGGCTACACTTTTGACAATGAGGTAGTCCTCCCT
CAGTATTTTTATATTTTGGAGGTTTTGAGGGGGCCATATGGCAAAAATAAAAAGTGGAAGTAAGCTGAAAAGGAGAGAGGAGAGAGA
GAGAGAGAGAGAAAATGCTAAGGGGCAGCAAGGCTGAAACCCACCCTCCCCTGTGTCTCCTGAATAAAGCTGCATTGCAGAAAAGCT
CTCTCCAACCAGAATCCACCTTTCCTTCACGCCAGCCCAACTCTCCATCATGGTTGATTTAAAGAAAGAAAACAAAACACAGGCCAA
GGGTATCACCATCCTCATCACCAGGGTGGTGTGTCCCTTCCTCAGATTACAGGGTGACAGGCTACAGCCCTCTGTCTGCCTAGGGTG
TGATCGTCTCATATAAAGCCTGGGTTTCCAGGCCACCATGGCCAGGTGCTAACCTCCTGGTTCCTGTATTCCAGGATGATCCTGTTT
GCTACCTAAAGAAGGCCTTTTTTCTGGTACAAGACATAATAGATGAGACCATGCGCTTTAAAGACAACACCCCCAATGCTAACGCCA
CCGAGAGGCTCCAGGAACTCTCCAATAACCTGAACAGCTGCTTCACCAAGGACTATGAGGAGCAGAACAAGGTAGGGTGGATTCTGG
GGCTGGAGCACCAGAGAAGGGCAGAGGGAGGCTACAGAGGTCCCACCCTGCCTCATATGCTCACCGAGCCTGAACCTTACTAACTTA
CTCCCTTGCCCATTCATTTCTTATGCCATCCGCCTTTATCAAGCATTCATTAGACAGAAGGCACAGTGCTGGACCCTGAGGATATCC
TTGGAACAAGAAATTCCCCTTCCCACCCTTACAGGGCTTATAGCCAAGTGAGGTAGACAGCTGTTAAATGATGACACCTATAAATAT
GATCAGAAGGGCTCTTCATTCATTCAGTAAAACCTTACTGAGTGACCATCAAGGGATGTTCTGAACACCAGAAATGAGCTGTGATTT
TAAAAAGGGGGGGGGCAAAAATCCCCACAATCATGACGAGAGGAGGGGTGCTGAGGCAGCTAAAATGAGCAAGTATGTGAATGGACA
ATGATGCTAACAGCATTGGAGAAGTGTAGAGCAGCAGGTGAGGGGCGCCCTGGGTGACAGTGAGAGGAACAGGAACCCGAGACTCTG
AGTGTGCGGTGAGGAGCTAATGTAATGAGGATGCAGAGGGGAAGAGGCAAAGGCAGCTTCTAAGCAAGAAAACTTTAGCCGGCCCTC
CAGCGTGAGGAAGAGCAGTAAGAGCCACAGACAGAGAAACAGAGTGGGGAGTGCTATGCTCAAGCCCTAGGGTAACCAGAAAGGTTT
GCATTCAGAAATCCAAAGCGGCTCACCATGGCCCAGGCATGGGGTGTGGACAGGAGGAAGCAAAGTCAGGAAGCACTGCATTCATGC
ATGGTGTCAAAGAGGTGGGCTTTTTGTCTAAATTTGAAAGAAGCCACCATAAGCTAGTGAATGACACGATGTCATATATGATGGAAG
ACCATCTGGTGGTGAGGACGCTGTGGAGAGCAGGAATGAAGGGCAGGAGGCCAGCAGGAGGTTCAGCCAAGCTGGGCATGGTGGTGA
GTTGAATCCGAGGGGAGCAGGGGAAATGGAGGTGAGTGGGTGTGATCCCTCCAGACAGACAGGACTCCATGCTTCCTGGCAGAAGGG
GAAGGGTGAGTGAGGATGCTCTCATTTTGAGTCCCTGGAGTTCTCACTGATGTCAAAATAAGGATGACCTCAGGCAGAGTAGGTTTG
AGAGAAGAGCTCATTTTGGTGTGAGGCACATCTGACAGCTGCAGGCGCGGCTCATGACTGTGAATGCTGGACATACAGATGCTGTTT
GCAGAGCCACAGAACTGCACAGCCCTGCCCATCCTTCTCACCCTGGTGAGAAGAGAAGGGGATCCAGGATCCTGAATCCTGAATGCA
ATGCAGCCTGAGGTAGGAGGAAAACCTAGAGAGGGCAGGGCTTCCAAATGGAAGGAGCCAGAAGCTTTATCAAAGGAGGCTGAGAAG
TCAATTAAGGTAGTGTCCATTGGTCTGTCTCACTTGGCAGCATGGAGATTGCTGTTGGAGGCAGGTGGGGAGAAAAGAAAGAGAGGA
AACAGAGACCACAAAGTAGAGGAGCTTCGCTCCAAAGGGGACAGAGAAGCAGATGTTCTGGGGTGGCTTTCTGGTGGAGGAAAGGCT
GGTTCTTTACCATGCAGAGATTCACCCGGAGAGCTTATTTAAAATGCAGAGTCCCAGCATCACTGCTACGGACTCTACTTTCCTGAG
CTCATAGGTGAGCCTGGGAATCTGCATTTTGCGAAGCTGCCCCATCAGTGGTTGAGATAGAAGCCCCTCAGTCTTTCCTGGGCCGGT
GACCATTCACTCAGTCCATGCTGTGGGGTGGCTCAGGGGAAAGACAGTGTAAGCCCAAGGAGCCAAGTCTAGTCACATGGCTCAGAA
AAGGAGTATGAGCTCCGGAACCAGCAGGCTCCACACTGAGAAAGCTTGGGTGTCAGGTTCTAGGGGAGGATGCCTGGTTTCTTCTAC
AGCATATAAGGCAGGTTTGAGGGAAACAGCTTTATAAAGACACATGCAGGACAGAGCCAGCTGGCCTGACTTAACAGCTCCTGGGAA
AGGCTGGCTGCTGTTGCTCTAACATCACAAACAAGGAGATGAATCCACAGAAAGCCGGTCTATTCTCACCTAGCTGCTGAGTGATGA
ATCCAAGCCCAGGTCATTTCATTCATTATTTGCCTAGTAAGTGTTTATGAGCCTGCTATGGGCGGGCCAGAGAGATGCAGTGATGTA
ATCAAGGGAGCTATAGTAGCCGTAGAAAACCTATTGTTCTTCCCAGAACAGCTCAACTTCAAGGGGGAAAGTTGGCATTCTGGAAAT
AAGCCGTAACACATATAGCTTGCTGTGAAAGGTGAGGTGGACCTAGGGCAGAACCCTGCAGCACTGCAGTATTTAGAAGGAACTCTC
TGGCACAGAGCCTGAAGGAGCAGCTGGTGAGGAAGGAAGAACTCTGGAGAGCCTGGCTTCTCCGAGCTGCATGGGGTGTGAGTGTGA
GCACCTGGGCCCAAGGCAGAGGAGAAAAACCACTCAACAGGCAGGCCAGGGAGTCCTCAGCTCTTCCTCCAAAATGTGGCACCAGCC
AAAGACCTCATCTCCTCTGATCTCCCCAGGTCTGCTCTCTACACCGTACCAAGATGATTTTCCTAAAATACAGGTTTTATACCATGG
TTTTAAACCTCCCCAGCAGGTTTTCAGTTTCCGGTTGGCTCTGTGAATTACAAGTTCCTGGCAGAGTCAACAAGTCCTTGGCCTTGG
CAAAGCCCTTTCAGCTGCCACTTTCTTCCCACGTGGCAAAGCTGCACCCGCTCCAGCCTTCTTTCACTCTTTTGCTGACTCATGCTT
TCCTGCCCCAGGGCCCTTGCATCCTCTATCATCTCTGGCCCCACTTCCTTCTCTTCCCTCGCTCATCTTTTAGCTGTTCCTTTGGAG
ATCTTCTCCCTGGGCCCTCATCTATCGCCCATTTGTTACACACCTTCTGATGGTTCAGGACACTCTCTTGAGAGTCACTGTGACTTG
TAACAATGTGTCTGCTGGACTCAGTGCCCAGCTTGGCCAGGAACCACTTCTCTTTTGTTCATTGCATGATGCCCAGGTAAGTTACCA
TATATCTGGCACTTAAACGTGTGTTAGATGGATCTCTGACTAAATGACGCATCTCTCAACCTCAAGACTGATGCTGCTGCAAAGCAG
AGAAGGAAGGAAACAAAAGGGGGAAAGGAAATGCCTGTGAGAAACCCCAAGAGAGTTTGACAAAAATACCAGTGAGAGGTAGCCCAA
TGTTCATGCTTACCAAAGGGGGCTCCACTCTCCTCCAGGCCTGTGTCCGAACTTTCCATGAGACTCCTCTCCAGCTGCTCGAGAAGA
TCAAGAACTTCTTTAATGAAACAAAGAATCTCCTTGAAAAGGACTGGAACATTTTTACCAAGAACTGCAACAACAGCTTTGCTAAGT
GCTCTAGCCGAGGTAAGCACAGAAGGGGTCAGCTACAGGGAGGGTGTGTGCAAGGTGAGACGGGGCTAGGGGAATCCCAGAGGCAGC
CTGGGTGCTAAGTGTGTTTCTGTGTTTTGTGTACTCGTGTGGCTTCATGTACCTTAAGGTCCTGGGCACATGTCTCTGCTGACATGT
GTGTGCATGTACACTGACTTATGCCTGCATGTACTGTCTCACTGCCAAACTTCATGTGGCAGCATCAAGAAGGCCCCTGCTGGTAAC
TGTGATGGAAGTGAATGGGTAAGGGGACCTTTCTTAGAAAGCCATATAGGCATGGTGGCGTCCCCCGGGCTCTGCAGGCTGGCTGAA
TAAATGGGGGTTCCGGAAAGAGCAGGTGCTCCACCTGGCAATCAGGGTCCTTGCTCCAGGAGCTCTGCTGCAAAACACCAGGATAAC
CTGGCTCCAGAAGTCCCCAGGATGCCTTGGATGTTCCCTCAAGGGATAAGGCTGGATTTGAAGTCTGAACATCATTCAAACTCTCTC
TACCTGTGCACTCTGAGTGTACTCGTCTCTGCATCCTGTGCCACGTGTGTCCTGCGTTCAGCCTCGCTCCCCAAGGCAGGGGCTGAG
GTCCTGCAACCAGACAATAGCCACATCCAGCCTTAGTGCTTCAATTCCAAAACCCTGAACTTTGGCTGCTTTCCAGCCCTGCCTTGC
CCTGCCTGTGGCAGTGGCTGAGGTGGTTCCACTCCTGGCTGTGCATGAAACCAGTGTCTTGGAGGCTTCTGAGTGTCTTGTGTTAAT
CCAGGAGCTATTGCTCAGCCCTGTGGCCTGGGTGCTGCTAACCCCTGGCTTGGCTTCTGGCCAGCTAGAGCCCCAGTACACTGTCCT
GTGGTCTTCCTCACCTCAGTCCTTCTCTGTCTGTCTGTACCTTGCTTTTGGTTCCTAGTCCATATCCCCTGCGTATTGATCCCTCCT
GCCTGAGTTCTGTGTCATGATCGTTTATTACTGTCCTGCCCTCTCCACCGCCCCAGGGCTGTGTTAGAAGAGGGACCCCCTTCCCAT
GCCCCAGGGCCATATGCCCCTCTTAGCCCCTCTGGGGGAGCTGTGCTGGAGGCTGGTGACTCTATCTCCTCCCCATCTTTCTCTCTC
CCCCTCTCTGTGGTTCTTTCAGATGTGGTGACCAAGCCTGATTGCAACTGCCTGTACCCTAAAGCCACCCCTAGCAGTGACCCGGCC
TCTGCCTCCCCTCACCAGCCCCCCGCCCCCTCCATGGCCCCTCTGGCTGGCTTGGCTTGGGATGATTCTCAGAGGACAGAGGGCAGC
TCCCTCTTGCCCAGTGAGCTTCCCCTTCGCATAGAGGACCCAGGCAGTGCCAAGCAGCGACCACCCAGGAGTACCTGCCAGACCCTC
GAGTCAACAGAGCAACCAAACCATGGGGACAGACTCACTGAGGACTCACAACCTCATCCTTCTGCGGGGGGGCCCGTCCCTGGGGTG
GAAGACATTCTTGAATCTTCACTGGGCACTAACTGGGTCCTAGAAGAAGCTTCTGGAGAGGCTAGTGAGGGATTTTTGACCCAGGAA
GCAAAGTTTTCCCCCTCCACGCCTGTAGGGGGCAGCATCCAGGCAGAGACTGACAGACCCAGGGCCCTCTCAGCATCTCCATTCCCT
AAATCAACAGAGGACCAAAAGCCAGTGGATATAACAGACAGGCCGTTGACAGAGGTGAACCCTATGAGACCCATTGGCCAGACACAG
AATAATACTCCTGAGAAGACTGATGGTACATCCACGCTGCGTGAAGACCACCAGGAGCCAGGCTCTCCCCATATTGCGACACCGAAT
CCCCAACGAGTCAGCAACTCAGCCACCCCCGTTGCTCAGTTACTGCTTCCCAAAAGCCACTCTTGGGGCATTGTGCTGCCCCTTGGG
GAGCTTGAGGGCAAGAGAAGTACCAGGGATCGAAGGAGCCCCGCAGAGCTGGAAGGAGGATCAGCAAGTGAGGGGGCAGCCAGGCCT
GTGGCCCGTTTTAATTCCATTCCTTTGACTGACACAGGCCATGTGGAGCAGCATGAGGGATCCTCTGACCCCCAGATCCCTGAGTCT
GTCTTCCACCTGCTGGTGCCGGGCATCATCCTAGTCTTGCTGACTGTTGGGGGCCTCCTGTTCTACAAGTGGAAGTGGAGGGTAAGG
AGGACCCCAAGAGAGGAAGACATCCTATGGGGCAGAACAAAGGCTGGGTAGGGTACAGGCTGGAGGCTAACCAGGTTCCAATACGTT
ATGCAGTTGCTGGAAGGTGGGGATGACAGCCCGAACGGTAGACGGTAGTGATGGAGTGTGGCTTCACACAGGAGCTGCCAGGTTTGG
TGGTGTGCATTTAACCCTAGCAATCCAGAGGTACAGGCAGGCCTCTGATCTACTGGTCAACAAAGCAAGTTCTAGGCTAGCCGAAGC
TACATAGATCCTGTCTGAGAAAGTAATTAATCAATTAATTAATGAGGTCTCCAGCCTCAAGATCATTAAGAAAGGATGAGTTCTAGG
CTAGTCAGAGCTTCATAGAGAGATTCTGTCAAATAAATAAATATATAAATAAATAAATTTAAGAAGTTTCCAGACTCCTTGAGATTA
TTGAGAAAGGATGAGAACAGAATCCTGTAGAAAGGAGAATTCTTGGCTGGGAACTCAGGAGGCAGAGAGGCAGAGAGAGAGGCAGAG
AGAGAGGCAGAGAGGCAGAGAGGCAGAGAGGCAGAGAAGCAGAAAGGCAGAGAGGCAGAGGCAGGTGGATCTCTGTGAGTTTGAGGT
CAGCATGATCTACAGAGTACCAGGGCAGCCAGGGTTGTTATGCAAAGAAACCCTGTCTTGAAAAAAATGAGAGAGAGGGAGAGGGAG
AAGGGATGGGAGACGGAGAGGGAGAGAGGGAGAGAGAGGGGGGATATTCTTAAACAACCAAGTGGGAGAGGGAAGGAAAGGGGAAGA
GCTTGAGGGAGCTCTGGGAGCTTGAGGGCCTATAGGTGGTATTCTACATGGGAGGGGACAGGAGAAGGCGCACCCATCCTCAGAGGC
CCAGGAATCTCCCCCAGCTGTGCATGCTTTGGGGTAGTGGGAGGAATTCTAGCAAGTCTCTCAGTGATGCTCAGGGGCATGCCAGCG
CCTCAGTGAGATAAATCTTTCTTGTTCTCAGAGCCATCGAGACCCTCAGACATTGGATTCTTCTGTGGGGCGACCAGAGGACAGGTG
AGCAGTTGGAGTGGGGCTATAGGAATCACTGGATGTCTGTCTTGGGACCTGCTTCCCCTCAAGACTGAAAGAGAGGCTTCTCCCCCC
TTGCCCTAGGAGATAAGCTGCAGAGCAGGGTGGGTACCAAGAAAGAAGGGCCCCTCTTCTTCCCATGAAACAGTGGAGCTTTCTCCT
GATTTCTTCCTGGAGTTGGGCAGTTCTGGGTCTTCTAAATCTCACATAGATGGAATCCACGTGCAGGGTTGGGGGTAAGCTAGGAGG
TCCCCTCAGTCCCATGCTCCTGCTGAAGTCTTATGTCAGCCCTGTGTTCTGCCTGCAGCTCCCTGACCCAGGATGAGGACAGACAGG
TGGAACTGCCAGTATAGAAAGGATTCTATGGTAAGGTTCTGATTTTGATATCTCTCTATCCCTTAAAAAGAACTATCTCCAGGTACC
CAGTCCTGTTGTAGAAACCACCTGATGCCCAAGAGGCCCACACATTTCAACTTCCTCTCCCCGTCCCACTCCCAAGCCCATACATTC
ATCATCACGTCTAGCTAGGCTCCTCTCTTCCAGGCCTCAGGTCTCCTCTTGCTCCTGAATCCCTGCCCTGGACCAGTTACTGCAACC
TCTGCATTATGTTTCTATTCTTGCTGTTGCTACATCTGATCCCAAAGCAACCACATTCTTCCTCCTCTAATGAGGCTTTTTACAAGG
TCTTTACCACCCTGGAGAATTTTCATTAGCCTTTGCATGGCCGCCAGCCCCCTTCAAATACGTGCATTTCCAGCTTGGTGCTGAGCC
CTGACCTCCTCAGCATCCTCTTCTTGCCAAATTGTGCCTTATCTGGTCCTACTCCCAAGCCAAGGTTATTGCTGCCTCCCTGATTTA
GGATCATTGTGCCATAGAAATTTAGTCTTAGGAGAGCTGTCGAGAGGTCATTTGGCCCAGTGTCCCAGCCTAAAAGGGTTACTCTGC
ATCCCCCACAGGGAGTCAGCCAGCTTCCTGCACACACACACACACACTCCCAGTTCCAGGCAAGTTCTCTGCATGCCAGTCTGCTCA
ACTGGGGTCACAACATTGGGGTCACAACACACAACACTGGGGGAGTTCCTTCCTTATGTAGGCAAAGCTGCCTTCCTTTCTTCCCTT
CCCAGACCAATCCTCTCTCATCTTCTCATGAGGCCTGATATTTAAAGTCAGCTATTCATGTCACCTTTTAAAATTCTCTGTGTTAAG
TGTGGCCAAGCCCCTCAAACATTCCTTATATGATATGGTTTTCAGACCCCTCACCATCCTGGACACACTCGTTTGTCAATGTCCCTC
TGAAAATGTGGCGCCCAGCCCTGGACACAGTACTCCAGATGTTGTCTGACCAGCTCAGAGTACAGTGGGACGGTTGTCTTCCTTGAT
CTGGACAGTACTCTTCTACTCGTGCAGATTAAGATCACATTAGTTTTAACACCTGCATCATATATTGTCATATGTTGAGCTTGTAGT
CTATTAAAAACCCCAGTTCTATTTCCTGTGAACCTTTGTCCAGTAGACCGTCGCCATCCCATACTCCCATACTTGGACACAACCGTT
TTAGCCAAAGTGTAGCTGGTGCTCACCTTTGTTAAAACTCCTTGTTGTTTTCTGCCCATCCCCTGAGCCTACTGAAAGTGTTTTAGT
TCCTAATTTGGTCACTTTATAACTCCTGGTTGGGTCCCCTGCACATTAATGCGTGTCTTTTGTTGTCCTTGCCCACGCTATTGGTGG
AGATCTGACCTGTATTCTTCCTCTACACCCACCCACACCAGCCTATCCAGAAGCCTTCCCTTCCTAAAATATTCCACATCCCTCTGG
TTGATCTTTTAATTCATTCTGCTCTGTTGTATATGGCTGATGCTATATTAATCCACATACATTTGGATGTTGTTTTATGTATTTTAA
ATCATATTTTATGTATATATGTAAATTTTGAACTACTAAGAAGATTGTAAGCCCCTAGAGGGCAGGGACTATCTTCCTTTTTATTTT
TTTTCCCCTTCACTTCATTAATTCTAGTACAGACAGTGCCCCCTCTATCCTTGTCAAAAACTTAGTAAATGGTGGTAGCTGTTTTCA
GGTCTGAATCACCAACTAACCCCGGCCAGATTTCTCTAACTGACCAGCCTTCTGGCTGACCAATTAACTAGCCAACTAATCAACTCA
TCTTACCAGCTAGTTGGCTAACTGACTACTTGGCCAACTAGCTGGTAACTACTTAACTGACTAGTTCTTGATTGATGAGCTGATTGG
CTAACTAGCTGGCAGAGTGCTTAGTTGATTTGCTGACTGACTGGATGGCAAACTAGCTGTGTGATCGGTTGAAGTTTCCAGTTATAC
CCCTTGCCACATGTAAGCAAACTGTCCCATGTGTAGGATGATGTGTCTAAGCCCCTTTCCCAAAGGAAATATCACAGATGCTCAGAG
AGCCAGGGAGATTTATTTGAATTAAAGCAAGCAGCAACCCATATCTAGAATCCCGGGGACCCACTCAGCAGTAATGTGGGTCGAGAC
GGGTGAAATGAATTAGCTGGCTCACTGGAACAGCTACAAGGACACAGAGTCCCTCTGACCTGAATCAGAGAGCAAAGGTTGCTGGGG
ATTTGGGGGCTCAGTGCTGTGTCACCTTACCAAGGGCTACCAGGGTTCTTCCTCCCTGCCACCTGTATGACCTTGTCCTCGGCCCTG
GTTTCTCTGTACCCAGTTGTCCTTCCCATGCCTCCTAACTCTCAGACCTGCCCAAGATTTTACGTTCCTCACCTAAGGATTTCAAGA
TTTCACTCAGTCAGCGCAGATCTAACCTGATCCCCATTCCAGGCTCAAGGAGCCTATGGGAACTGAATGTCTGTGTCCTGATTGACC
AATACTCTGACAAGGAGATGAGTGGGGCACACAGAAGTGGGGACTAAGAAAAGGTCACCAAGTTGTAGAAAACTCAGGCCCTCGGCC
CCAGGAAACTGGGAGTTGGGTTAGAGGCTAAGGTCATGCCCTCTGGGGCCTTCTTCCCGATGGGCCCTGCCAGAGCCTGTGGCCTGG
TTGTACCAGTATGGCTCATGCCCAGTTTATAGTTTCTTCTTGTTTCCTATCCAAAGCATGGGAATAGGCTGGTGGCCAAAGGCTGTA
GGTCTCATTTTCTTTCATACCCATCACAGGCTTGACCAGTGGGGCTATGGACATTACTGGGGAGGCGCCATACAGGGTCCAAAGTCA
AACTGTTGGCTGAAGTCCAGGGGATTTCTGCTATGGAACAAAGAGTTCTGAGGATACTGGACAAGGGGACAAAGATGGGGGTGGGGT
GGGGAGAGGGCAGGGCCCTGGCTCCCATGGATGACAAGAGGCAAGTCCCTCATCCCATCTGGGAGGAGGCTAGGCTGCTTGGGGTGA
GCATGCCTGTTGACAGGGCAAGCCCTCCCTTTTCCTGGCATTGTCTCTACCCTCTGCCCCTCAAGCCTGAGTTCTTTCTATTCAAAT
GCCTTTGCCCAAGTGTCTTCTGTGCAGATACCCTCTAAGGGATGGTATGAGAGTTGGCAGAGATCACCTTAGGTCATTGGTCTAAGA
CTACTGAGATGGCCTTACCTGAGAACTAGCCTAGTAGGGGCATTCAAGAGCTTGTTCCCTGCTCCCATCTGCTAGAGCAATAGCCCT
CTCTCTCTCTTGGCCTCGACCTCCTCCAGTCTTAGTCCCTCATTTTGTGGTCACTACTCCCCCAGGAGCAGAGGTAGGAGGTGAGGC
CAGGCTATCCAAAGGCTTGGACTGCTGCCAGGCAAAGCTGCCTGGTCTCTCTGTGGTGTCTGGGGTCGCCTAGAGGGCCAGGAAAAT
GACCAGAGGAAGTCTTGCCTTAAGTCAGCCAACAACCCACAGGAATGTCACACCTCCCCCAACTATGTCCTTAGCGACCCTCCCCCA
CACTCCCCTGCTGACCAGCCTTCTTCCCTTCTTTCCCCTACAGCTGGGCACACAGGACTATCTCTTTATGGAAGGAGACATATGGGA
ACATCCACCACTACCCTCTCCTACCATCTTCCTGGGAATGTGGCCTACCACTACCAGAGCTCCTGCCTACCAAGACTGGATGAAAGA
AGCAGCTTTGATGGCGTCTTTCCATCCTCACCCTTAGACTCTCAACCAAAGAGAAAGGGCTGGAGGATGCCCCCCACATACTGCCAC
TATTTATTGTGGGCCCTGGAGGCTCCCTGCATTGGAGGAAGGGCAGCTCAGCAGCTCAGGACCCTTTCCCTTAGGGGCTGCTTCCTC
CCCTCAAAACCAGAACCTGGCAAGGGACTCACTAGCCTGGATGGCCCATGGGAGACCAGGACAGATGAGAAGGAGCAGAAGAGCCCT
GTGCCCAGAAGACCCAACTGGTGCCAAGGAATCCCAGCATGGACAGGCAGGGACCTGTTTCCCAAGAAGAGAGCCTGATATTCAAAG
GGTGGGACAGCATCTGCCCGACTTCCCGTAAAGGCATAAAGGCACGCAGCCCAAAAGACGGGAAGAGGAGGCCTTTGGCTGCTTGTG
TTGACAGCTTAAAGGGGTCTACACCCTCAACTTGCTTAAGTGCCCTCTGCTGATAGCCAGGAAGGAGGGAGACCAGCCCTGCCCCTC
AGGACCTGACCTGGCTCATGATGCCAAGAGGAAGACAGAGCTCTAGCCTCGTCTTCTCCTGCCCACAGCCCCTGCCAGAGTTCTTTT
GCCCAGCAGAGGCACCCCTCATGAAGGAAGCCATTGCACTGTGAATACTGAACCTGCCTGCTGAACAGCCTGTCCCATCCATCCCTA
TGAGTGACCATCCGTCCGAATGTTCTCCCACTTCCTTCAGCCTCTCCTCGGCTTCTTGCACTGAGCTGGCCTCACGTGTTGACTGAG
GGAGCCCCTGAGCCCCAACCTTCCCCTGCCTCAGCCTTTGATTGTCCAGGGTGAAGCTGTGGGAGAACCGCCTGGGCTACCAGTCAG
AGCTGGTCTTTGGGCTGTGTTCCTTGCCCAGGTTTCTGCATCTTGCACTTTGACATTCCCAGGAGGCAAGTGACTAGTGGAAGGGAG
AGAGGAAGGGGAGGCAGAGACAAAGGCCACAGGCAGAGCTATGAATGAGAATGGGTCTTGAAAATATGTGTGCACCCCTAAGCTTGA
AATTGATCTCTATACTCTAGCCCCTCAGCCAGCCTCCTTCCTGTTGTCTGAAACCTGGAGCTAAGCAGGTTGTCCTGTCACAAGCTC
TGGGGACTGAGCTCCATGCTCCAACCCCACCCTCTTCTGACCTTTGTTCTCCAGACCTGACCCAGGTAGGCAAGGGTACCCTCCCAG
TCTCACCTACCATACTGTGCCATCTCTAGCCAAGCAAGCCAGGTTTAGAGAAGGGTCAAAAAAAAAAAAAAGGGTTGTTTACTTCCA
ACTTGTTCTGATGCCCTCTGTTTCCCAGGCCAGGCTTGTCTGTGGTGACCTGGGCATGGGTGACAGGGCTCTCATTTGCCCCTTGGT
CTCTTTATGCTGCTGAGTCCCCCTTTCCTGCCCTCCCTGGCTACTGGGTCAATAATCTTTCAGGCCATGAATCTGGGAGGAGAGTGG
TCTGTAAGCTCCATCAGCCCTGTCCTGAGACAGCAGGGGGGAAGGACACTGGAGACTTTCTTGTGGGGCTTACTTAGCCTTCTGGTT
ACAGACTATTTCCATGCTAGAAAATACATATTTTAAAATAGAAGGAAAAACACAGAAACAAAACAAAACAAGGCATTCTCTACCCCT
CCACCTTAAACATATATTATTAAAGACAGAAGAGAAAATCCAACCCATTGCAAGAAGCTCTTTGTGGGTGCCTGGTTACATCGGAGC
AGGGGAGCCTCAAATCCACCTTTGGAGCCGCCCCTGTGTGCATTAGGAACCCTTCTCTCCTCTGAGAAAGCTCAGAGGGAGCACTGC
CTCACAAACTGTGAGACTGCGTTTTTTATACTTGGAAGTGGTGAATTATTTTATATAAGGTCATTTAAATATCTATTTAAAAAATAG
GAAGCTGCTTTTATATTTAATAATAAAAGAAGTGCACAAGCTGC
MOUSE mRNA SEQUENCE: mR27-014.1 (Seq ID No: 32)
GGGAAAGTGAAAGTTTGCCTCGGTGCTCTCGGTGTCGCTGCGGCTCTCTGCATCCCAGGACAGCGGCGTGGCCCTCGACCGGGGCGC
GGGCTCTTCAGCCACTAGCGAGCAAGGGAGCGAGCGAACCAGGGCGGCCAACACGCCGTGCCGGGACCCAGCTGCCCGTATGACCGC
GCGGGGCGCCGCGGGGCGCTGCCCTTCTTCGACATGGCTGGGCTCCCGGCTGCTGCTGGTCTCCCTCCCTGGCTACTGGGTCAATAA
TCTTTCAGGCCATGAATCTGGGAGGAGAGTGGTCTGTAAGCTCCATCAGCCCTGTCCTGAGACAGCAGGGGGGAAGGACACTGGAGA
CTTTCTTGTGGGGCTTACTTAGCCTTCTGGTTACAGACTATTTCCATGCTAGAAAATACATATTTTAAAATAGAAGGAAAAACACAG
AAACAAAACAAAACAAGGCATTCTCTACCCCTCCACCTTAAACATATATTATTAAAGACAGAAGAGAAAATCCAACCCATTGCAAGA
AGCTCTTTGTGGGTGCCTGGTTACATCGGAGCAGGGGAGCCTCAAATCCACCTTTGGAGCCGCC
MOUSE PROTEIN SEQUENCE: mP27-014.1 (Seq ID No: 33)
MTARGAAGRCPSSTWLGSRLLLVSLPGYNVNNLSGHESGRRVVCKLHQPCPETAGGKDTGDFLVGLT*
MOUSE PANTHER CLASSIFICATIONS
FAMILY (SUBFAMILY)
MACROPHAGE COLONY STIMULATING FACTOR-1 (MACROPRAGE COLONY STIMULATING FACTOR-1)
BIOLOGICAL PROCESS
Immunity and defense (2.16.00.00.00) > Macrophage-mediated immunity(2.16.05.00.00)
Immunity and defense (2.16.00.00.00) > Granulocyte-mediated immunity(2.16.04.00.00)
Developmental processes (2.23.00.00.00) > Mesoderm development (2.23.10.00.00) >
Hematopoesis (2.23.10.06.00)
MOLECULAR FUNCTIONS
Signaling molecule (1.02.00.00.00) Cytokine (1.02.01.00.00) > Other
cytokine (1.02.01.99.00)
MOUSE GENE ONTOLOGY
No Gene Ontology
MOUSE PROTEIN DOMAINS (INTERPRO SIGNATURES)
No Domain Hit
MOUSE mRNA SEQUENCE: mR27-014.2 (Seq ID No: 34)
AGCTCGGCCAGGGGAAAGTGAAAGTTTGCCTCGGTGCTCTCGGTGTCGCTGCGGCTCTCTGCATCCCAGGACAGCGGCGTGGCCCTC
GACCGGGGCGCGGGCTCTTCAGCCACTAGCGAGCAAGGGAGCGAGCGAACCAGGGCGGCCAACACGCCGTGCCGGGACCCAGCTGCC
CGTATGACCGCGCGGGGCGCCGCGGGGCGCTGCCCTTCTTCGACATGGCTGGGCTCCCGCCTGCTGCTGGTCTGTCTCCTCATGAGC
AGGAGTATTGCCAAGGAGGTGTCAGAACACTGTAGCCACATGATTGGGAATGGACACCTGAAGGTCCTGCAGCAGTTGATCGACAGT
CAAATGGAGACTTCATGCCAGATTGCCTTTGAATTTGTAGACCAGGAACAGCTGGATGATCCTGTTTGCTACCTAAAGAAGGCCTTT
TTTCTGGTACAAGACATAATAGATGAGACCATGCGCTTTAAAGACAACACCCCCAATGCTAACGCCACCGAGAGGCTCCAGGAACTC
TCCAATAACCTGAACAGCTGCTTCACCAAGGACTATGAGGAGCAGAACAAGGCCTGTGTCCGAACTTTCCATGAGACTCCTCTCCAG
CTGCTGGAGAAGATCAAGAACTTCTTTAATGAAACAAAGAATCTCCTTGAAAAGGACTGGAACATTTTTACCAAGAACTGCAACAAC
AGCTTTGCTAAGTGCTCTAGCCGAGATGTGGTGACCAAGCCTGATTGCAACTGCCTGTACCCTAAAGCCACCCCTAGCAGTGACCCG
GCCTCTGCCTCCCCTCACCAGCCCCCCCCCCCCTCCATGGCCCCTCTGGCTGGCTTGGCTTGGGATGATTCTCAGAGGACAGAGGGC
AGCTCCCTCTTGCCCAGTGAGCTTCCCCTTCGCATAGAGGACCCAGGCAGTGCCAAGCAGCGACCACCCAGGAGTACCTGCCAGACC
CTCGAGTCAACAGAGCAACCAAACCATGGGGACAGACTCACTGAGGACTCACAACCTCATCCTTCTGCGCGGGGGCCCGTCCCTGGG
GTGGAAGACATTCTTGAATCTTCACTGGGCACTAACTGGGTCCTAGAAGAAGCTTCTGGAGAGGCTAGTGAGGGATTTTTGACCCAG
GAAGCAAAGTTTTCCCCCTCCACGCCTGTAGGGGGCAGCATCCAGGCAGAGACTGACAGACCCAGGGCCCTCTCAGCATCTCCATTC
CCTAAATCAACAGAGGACCAAAAGCCAGTGGATATAACAGACAGGCCGTTGACAGAGGTGAACCCTATGAGACCCATTGGCCAGACA
CAGAATAATACTCCTGAGAAGACTGATGGTACATCCACGCTGCGTGAAGACCACCAGGAGCCAGGCTCTCCCCATATTGCGACACCG
AATCCCCAACGAGTCAGCAACTCAGCCACCCCCGTTGCTCAGTTACTGCTTCCCAAAAGCCACTCTTGGGGCATTGTGCTGCCCCTT
GGGGAGCTTGAGGGCAAGAGAAGTACCAGGGATCGAAGGAGCCCCGCAGAGCTGGAAGGAGGATCAGCAAGTGAGGGGGCAGCCAGG
CCTGTGGCCCGTTTTAATTCCATTCCTTTGACTGACACAGGCCATGTGGAGCAGCATGAGGGATCCTCTGACCCCCAGATCCCTGAG
TCTGTCTTCCACCTGCTGGTGCCGGGCATCATCCTAGTCTTGCTGACTGTTGGGGGCCTCCTGTTCTACAAGTGGAAGTGGAGGAGC
CATCGAGACCCTCAGACATTGGATTCTTCTGTGGGGCGACCAGAGGACAGCTCCCTGACCCAGGATGAGGACAGACAGGTGGAACTG
CCAGTATAGAAAGGATTCTATGACCCCTCACCATCCTGGACACACTCGTTTGTCAATGTCCCTCTGAAAATGTGGCGCCCAGCCCTG
GACACAGTACTCCAGATGTTGTCTGACCAGCTCAGAGTACAGTGGGACGGTTGTCTTCCTTGATCTGGACAGTACTCTTCTACTCGT
GCAGATTAAGATCACATTAGTTTTAACAGCTGCATCATATATTGTCATATGTTGAGCTTGTAGTCTATTAAAAACCCCAGTTCTATT
TCCTGTG
MOUSE PROTEIN SEQUENCE : mP27-014.2 (Seq ID No: 35)
MTARGAAGRCPSSTWLGSRLLLVCLLMSRSIAKEVSEHCSHTHGNGHLKVLQQLIDSQMETSCQIAFEFVDQEQLDDPVCYLKKAFF
LVQDIIDETMRFKDNTPNMJATERLQELSNNLNSCFTKDYEEQNKACVRTFHETPLQLLEKIKNFFNETKNLLEKDWNIFTKNCNNS
FAKCSSRDVVTKPDCNCLYPKATPSSDPASASPHQPPAPSMAPLAGLAWDDSQRTEGSSLLPSELPLRIEDPGSAKQRPPRSTCQTL
ESTEQPNHGDRLTEDSQPHPSAGGPVPGVEDILESSLGTNWVLEEASGEASEGFLTQEAKFSPSTPVGGSIQAETDRPRALSASPFP
KSTEDQKPVDITDRPLTEVNPMRPIGQTQNNTPEKTDGTSTLREDHQEPGSPHIATPNPQRVSNSATPVAQLLLPKSHSWGIVLPLG
ELEGKRSTRNRRSPAELEGGSASEGAARPVARFNSIPLTDTGHVEQHEGSSDPQIPESVFHLLVPGIILVLLTVGGLLFYKWKWRSH
RDPQTLDSSVGRPEDSSLTQDEDRQVELPV*
MOUSE PANTHER CLASSIFICATIONS
FAMILY (SUBFAMILY)
MACROPHAGE COLONY STIMULATING FACTOR-1 (MACROPHAGE COLONY STIMULATING FACTOR-1)
BIOLOGICAL PROCESS
Immunity and defense (2.16.00.00.00) > Macrophage-mediated immunity (2.16.05.00.00)
Immunity and defense (2.16.00.00.00) > Granulocyte-mediated immunity (2.16.04.00.00)
Developmental processes (2.23.00.00.00) > Mesoderm developmemt (2.23.10.00.00) >
Hematopoesis (2.23.10.06.00)
MOLECULAR FUNCTIONS
Signaling molecule (1.02.00.00.00) Cytokine (1.02.01.00.00) > Other cytokine
(1.02.01.99.00)
MOUSE GENE ONTOLOGY
No Gene Ontology
MOUSE PROTEIN DOMAINS (INTERPRO SIGNATURES)
No Domain Hit
MOUSE mRNA SEQUENCE: mR27-014.3 (Seq ID No: 36)
GAAAGTTTGCCTCGGTGCTCTCGGTGTCGCTGCGGCTCTCTGCATCCCAGGACAGCGGCGTGGCCCTCGACCGGGGCGCGGGCTCTT
CAGCCACTAGCGAGCAAGGGAGCGAGCGAACCAGGGCGGCCAACACGCCGTGCCGGGACCCAGCTGCCCGTATGACCGCGCGGGGCG
CCGCGGGGCGCTGCCCTTCTTCGTGGATCCAGGTTCAAATCCCAGAATCTAGATGATGGTTCACAACCATTCATACCTCCAGGTCCA
GGAGATTCCAAGAAGGAAGCACATAGAGTGTGTGCATGCAAGCAGGCAAAATATTCATAAACACAAAACAAAAACAACATGGCTGGG
CTCCCGGCTGCTGCTGGTCTGTCTCCTCATGAGCAGGAGTATTGCCAAGGAGGTGTCAGAACACTGTAGCCACATGATTGGGAATGG
ACACCTGAAGGTCCTGCAGCAGTTGATCGACAGTCAAATGGAGACTTCATGCCAGATTGCCTTTGAATTTGTAGACCAGGAACAGCT
GGATGATCCTGTTTGCTACCTAAAGAAGGCCTTTTTTCTGGTACAAGACATAATAGATGAGACCATGCGCTTTAAAGACAACACCCC
CAATGCTARCGCCACCGAGAGGCTCCAGGAACTCTCCAATAACCTGAACAGCTGCTTCACCAAGGACTATGAGGAGCAGAACAAGGC
CTGTGTCCGAACTTTCCATGAGACTCCTCTCCAGCTGCTGGAGAAGATCAAGAACTTCTTTAATGAAACAAAGAATCTCCTTGAAAA
GGACTGGAACATTTTTACCAAGAACTGCAACAACAGCTTTGCTAAGTGCTCTAGCCGAGGCCATGTGGAGCAGCATGAGGGATCCTC
TGACCCCCAGATCCCTGAGTCTGTCTTCCACCTGCTGGTGCCGGGCATCATCCTAGTCTTGCTGACTGTTGGGGGCCTCCTGTTCTA
CAAGTGGAAGTGGAGGAGCCATCGAGACCCTCAGACATTGGATTCTTCTGTGGGGCGACCAGAGGACAGCTCCCTGACCCAGGATGA
GGACAGACAGGTGGAACTGCCAGTATAGAAAGGATTCTATGCTGGGCACACAGGACTATCTCTTTATGGAAGGAGACATATGGGAAC
ATCCACCACTACCCTCTCCTACCATCTTCCTGGGAATGTGGCCTACCACTACCAGAGCTCCTGCCTACCAAGACTGGATGAAAGAAG
CAGCTTTGATGGGGTCTTTCCATCCTCACCCTTAGACTCTCAACCAAAGAGAAAGGGCTGGAGGATGCCCCCCACATACTGCCACTA
TTTATTGTGGGCCCTGGAGGCTCCCTGCATTGGAGGAAGGGCAGCTCAGCAGCTCAGGACCCTTTCCCTTAGGGGCTGCTTCCTCCC
CTCAAAACCAGAACCTGGCAAGGGACTCACTAGCCTGGATGGCCCATGGGAGACCAGGACAGATGAGAAGGAGCAGAAGAGCCCTGT
GCCCAGAAGACCCAACTGGTGCCAAGGAATCCCAGCATGGACAGGCAGGGACCTGTTTCCCAAGAAGAGAGCCTGATATTCAAAGGG
TGGGACAGCATCTGC
MOUSE PROTEIN SEQUENCE: mP27-014.3 (Seq ID No: 37)
MMVHNHSYLQVQEIPRRKXIECVHASRQNIHKHKTKTTWLGSRLLLVCLLMSRSIAKEVSEHCSHMIGNGHLKVLQQLIDSQMETSC
QIAFEFVDQEQLDDPVCYLKKAFFLVQDIIDETMRFKDNTPNANATERLQELSNNLNSCFTKDYEEQNKACVRTFHETPLQLLEKIK
NFFNETKNLLEKDWNIFTKNCNNSFAKCSSRGHVEQHEGSSDPQIPESVFHLLVPGIILVLLTVGGLLFYKWKWRSHRDPQTLDSSV
GRPEDSSLTQDEDRQVELPV*
MOUSE PANTHER CLASSIFICATIONS
FAMILY (SUBFAMILY)
MACROPHAGE COLONY STIMULATING FACTOR-1 (MACROPHAGE COLONY STIMULATING FACTOR-1)
BIOLOGICAL PROCESS
Immunity and defense (2.16.00.00.00) > Macrophage-mediated immunity (2.16.05.00.00)
Immunity and defense (2.16.00.00.00) > Granulocyte-mediated immunity (2.16.04.00.00)
Developmental processes (2.23.00.00.00) > Mesoderm development (2.23.10.00.00) >
Hematopoesis (2.23.10.06.00)
MOLECULAR FUNCTIONS
Signaling molecule (1.02.00.00.00) > Cytokine (1.02.01.00.00) Other cytokine
(1.02.01.99.00)
MOUSE GENE ONTOLOGY
No Gene Ontology
MOUSE PROTEIN DOMAINS (INTERPRO SIGNATURES)
No Domain Hit
MOUSE mRNA SEQUENCE: mR27-014.4 (Seq ID No: 38)
TGAAAGTTTGCCTCGGTGCTCTCGGTGTCGCTGCGGCTCTCTGCATCCCAGGACAGCGGCGTGGCCCTCGACCGGGGCGCGGGCTCT
TCAGCCACTAGCGAGCAAGGGAGCGAGCGAACCAGGGCGGCCAACACGCCGTGCCGGGACCCAGCTGCCCGTATGACCGCGCGGGGC
GCCGCGGGGCGCTGCCCTTCTTCGACATGGCTGGGCTCCCGGCTGCTGCTGGTCTGTCTCCTCATGAGCAGGAGTATTGCCAAGGAG
GTGTCAGAACACTGTAGCCACATGATTGGGAATGGACACCTGAAGGTCCTGCAGCAGTTGATCGACAGTCAAATGGAGACTTCATGC
CAGATTGCCTTTGAATTTGTAGACCAGGAACAGCTGGATGATCCTGTTTGCTACCTAAAGAAGGCCTTTTTTCTGGTACAAGACATA
ATAGATGAGACCATGCGCTTTAAAGACAACACCCCCAATGCTAACGCCACCGAGAGGCTCCAGGAACTCTCCAATAACCTGAACAGC
TGCTTCACCAAGGACTATGAGGAGCAGAACAAGGCCTGTGTCCGAACTTTCCATGAGACTCCTCTCCAGCTGCTGGAGAAGATCAAG
AACTTCTTTAATGAAACAAAGAATCTCCTTGAAAAGGACTGGAACATTTTTACCAAGAACTGCAACAACAGCTTTGCTAAGTGCTCT
AGCCGAGATGTGGTGACCAAGCCTGATTGCAACTGCCTGTACCCTAAAGCCACCCCTAGCAGTGACCCGGCCTCTGCCTCCCCTCAC
CAGCCCCCCGCCCCCTCCATGGCCCCTCTGGCTGGCTTGGCTTGGGATGATTCTCAGAGGACAGAGGGCAGCTCCCTCTTGCCCAGT
GAGCTTCCCCTTCGCATAGAGGACCCAGGCAGTGCCAAGCAGCGACCACCCAGGAGTACCTGCCAGACCCTCGAGTCAACAGAGCAA
CCAAACCATGGGGACAGACTCACTGAGGACTCACAACCTCATCCTTCTGCGGGGGGGCCCGTCCCTGGGGTGGAAGACATTCTTGAA
TCTTCACTGGGCACTAACTGGGTCCTAGAAGAAGCTTCTGGAGAGGCTAGTGAGGGATTTTTGACCCAGGAAGCAAAGTTTTCCCCC
TCCACGCCTGTAGGGGGCAGCATCCAGGCAGAGACTGACAGACCCAGGGCCCTCTCAGCATCTCCATTCCCTAAATCAACAGAGGAC
CAAAAGCCAGTGGATATAACAGACAGGCCGTTGACAGAGGTGAACCCTATGAGACCCATTGGCCAGACACAGAATAATACTCCTGAG
AAGACTGATGGTACATCCACGCTGCGTGAAGACCACCAGGAGCCAGGCTCTCCCCATATTGCGACACCGAATCCCCAACGAGTCAGC
AACTCAGCCACCCCCGTTGCTCAGTTACTGCTTCCCAAAAGCCACTCTTGGGGCATTGTGCTGCCCCTTGGGGAGCTTGAGGGCAAG
AGAAGTACCAGGGATCGAAGGAGCCCCGCAGAGCTGGAAGGAGGATCAGCAAGTGAGGGGGCAGCCAGGCCTGTGGCCCGTTTTART
TCCATTCCTTTGACTGACACAGGCCATGTGGAGCAGCATGAGGGATCCTCTGACCCCCAGATCCCTGAGTCTGTCTTCCACCTGCTG
GTGCCGGGCATCATCCTAGTCTTGCTGACTGTTGGGGGCCTCCTGTTCTACARGTGGARGTGGAGGAGCCATCGAGACCCTCAGACA
TTGGATTCTTCTGTGGGGCGACCAGAGGACAGCTCCCTGACCCAGGATGAGGACAGACAGGTGGAACTGCCAGTATAGAAAGGATTC
TATGCTGGGCACACAGGACTATCTCTTTATGGAAGGAGACATATGGGARCATCCACCACTACCCTCTCCTACCATCTTCCTGGGAAT
GTGGCCTACCACTACCAGAGCTCCTGCCTACCAAGACTGGATGAAAGAAGCAGCTTTGATGGGGTCTTTCCATCCTCACCCTTAGAC
TCTCAACCAAAGAGAAAGGGCTGGAGGATGCCCCCCACATACTGCCACTATTTATTGTGGGCCCTGGAGGCTCCCTGCATTGGAGGA
AGGGCAGCTCAGCAGCTCAGGACCCTTTCCCTTAGGGGCTGCTTCCTCCCCTCAAAACCAGAACCTGGCAAGGGACTCACTAGCCTG
GATGGCCCATGGGAGACCAGGACAGATGAGAAGGAGCAGARGAGCCCTGTGCCCAGAAGACCCAACTGGTGCCAAGGAATCCCAGCA
TGGACAGGCAGGGACCTGTTTCCCAAGAAGAGAGCCTGATATTCAAAGGGTGGGACAGCATCTGCCCGACTTCCCGTAAAGGCATAA
AGGCACGCAGCCCAAAAGACGGGAAGAGGAGGCCTTTGGCTGCTTGTGTTGACAGCTTAAAGGGGTCTACACCCTCAACTTGCTTAA
GTGCCCTCTGCTGATAGCCAGGAAGGAGGGAGACCAGCCCTGCCCCTCAGGACCTGACCTGGCTCATGATGCCAAGAGGAAGACAGA
GCTCTAGCCTCGTCTTCTCCTGCCCACAGCCCCTGCCAGAGTTCTTTTGCCCAGCAGAGGCACCCCTCATGAAGGAAGCCATTGCAC
TGTGAATACTGAACCTGCCTGCTGAACAGCCTGTCCCATCCATCCCTATGAGTGACCATCCGTCCGAATGTTCTCCCACTTCCTTCA
GCCTCTCCTCGGCTTCTTGCACTGAGCTGGCCTCACGTGTTGACTGAGGGAGCCCCTGAGCCCCAACCTTCCCCTGCCTCAGCCTTT
GATTGTCCAGGGTGAAGCTGTGGGAGAACCGCCTGGGCTACCAGTCAGAGCTGGTCTTTGGGCTGTGTTCCTTGCCCAGGTTTCTGC
ATCTTGCACTTTGACATTCCCAGGAGGGAAGTGACTAGTGGAAGGGAGAGAGGAAGGGGAGGCAGAGACAAAGGCCACAGGCAGAGC
TATGAATGAGAATGGGTCTTGAAAATATGTGTGCACCCCTAAGCTTGAAATTGATCTCTATACTCTAGCCCCTCAGCCAGCCTCCTT
CCTGTTGTCTGAAACCTGGAGCTAAGCAGGTTGTCCTGTCACAAGCTCTGGGGACTGAGCTCCATGCTCCAACCCCACCCTCTTCTG
ACCTTTGTTCTCCAGACCTGACCCAGGTAGGCAAGGGTACCCTCCCAGTCTCACCTACCATACTGTGCCATCTCTAGCCAAGCAAGC
CAGGTTTAGAGAAGGGTCAAAAAAAAAAAAAAGGGTTGTTTACTTCCAACTTGTTCTGATGCCCTCTGTTTCCCAGGCCAGGCTTGT
CTGTGGTGACCTGGGCATGGGTGACAGGGCTCTCATTTGCCCCTTGGTCTCTTTATGCTGCTGAGTCCCCCTTTCCTGCCCTCCCTG
GCTACTGGGTCAATAATCTTTCAGGCCATGAATCTGGGAGGAGAGTGGTCTGTAAGCTCCATCAGCCCTGTCCTGAGACAGCAGGGG
GGAAGGACACTGGAGACTTTCTTGTGGGGCTTACTTAGCCTTCTGGTTACAGACTATTTCCATGCTAGAAAATACATATTTTAAAAT
AGAAGGAAAAACACAGAAACAAAACAAAACAAGGCATTCTCTACCCCTCCACCTTAAACATATATTATTAAAGACAGAAGAGAAAAT
CCAACCCATTGCAAGAAGCTCTTTGTGGGTGCCTGGTTACATCGGAGCAGGGGAGCCTCAAATCCACCTTTGGAGCCGCCCCTGTGT
GCATTAGGAACCCTTCTCTCCTCTGAGAAAGCTCAGAGGGAGCACTGCCTCACAAACTGTGAGACTGCGTTTTTTATACTTGGAAGT
GGTGAATTATTTTATATAAGGTCATTTAAATATCTATTTAAAAAATAGGAAGCTGCTTTTATATTTAATAATAAAAGAAGTGCACAA
GCTGC
MOUSE PROTEIN SEQUENCE: mP27-014.4 (Seq ID No: 39)
MTARGAAGRCPSSTWLGSRLLLVCLLMSRSIAKEVSEHCSHMIGNGHLKVLQQLIDSQMETSCQIAFEFVDQEQLDDPVCYLKKAFF
LVQDIIDETMRFKNTPNANATERLQELSNNLNSCFTKDYEEQNKACVRTFHETPLQLLEKIKNFFNETKNLLEKDWNIFTKNACNNS
FAKCSSRDVVTKPDCNCLYPKATPSSDPASASPHQPPAPSMAPLAGLAWDDSQRTEGSSLLPSELPLRIEDPGSAKQRPPRSTCQTL
ESTEQPNHGDRLTEDSQPHPSAGGPVPGVEDILESSLGTNWVLEEASGEASEGFLTQEAKFSPSTPVGGSIQAETDRPRALSASPFP
KSTEDQKPVDITDRPLTEVNPMRPIGQTQNNTPEKTDGTSTLREDHQEPGSPHIATPNPQRVSNSATPVAQLLLPKSHSWGIVLPLG
ELEGKRSTRDRRSPAELEGGSASEGAARPVARFNSIPLTDTGHVEQHEGSSDPQIPESVFHLLVPGIILVLLTVGGLLFYKWKWRSH
RDPQTLDSSVGRPEDSSLTQDEDRQVELPV*
MOUSE PANTHER CLASSIFICATIONS
FAMILY (SUBFAMILY)
MACROPHAGE COLONY STIMULATING FACTOR-1 (MACROPHAGE COLONY STIMULATING FACTOR-1)
BIOLOGICAL PROCESS
Immunity and defense (2.16.00.00.00) > Macrophage-mediated immunity (2.16.05.00.00)
Immunity and defense (2.16.00.00.00) > Granulocyte-mediated immunity (2.16.04.00.00)
Developmental processes (2.23.00.00.00) > Mesoderm development (2.23.10.00.00) >
Hematopoesis (2.23.10.06.00)
MOLECULAR FUNCTIONS
Signaling molecule (1.02.00.00.00) > Cytokine (1.02.01.00.00) >
Other cytokine (1.02.01.99.00)
MOUSE GENE ONTOLOGY
No Gene Ontology
MOUSE PROTEIN DOMAINS (INTERPRO SIGNATURES)
No Domain Hit
HUMAN GENOMIC SEQUENCE: hD27-014 (Seq ID No: 40)
TTGGGACGATCATAGAGCGCTAGCACTGAATCAGCCTGGAGAGCGCGGAAGGAAAGGGTCGGTCCGCAGAGGGCGCGGGGAAGGCAG
GGTGGGGACGCGGTGGAGCCCGCGCTCGTTTGCTGAAGGCTTGGAAGTGCAGCGCAGAAGACAGAGGGTGACTAGGAAGACGCGCGA
GCGGGGCTGGCCGGCCGGCGGGTGGGGGAGGGGAGGCGGGGGAAGGCGGCTGAGTGGGCCTCTGGAGTGTGTGTGTCTGTGTCAGTG
TGTGTGTGTGTGTGTGTATGTGTGTGTCTGGCGCCTGGCCAGGGTGATTTCCCATAAACCACATGCCCCCCAGTCCTCTCTTAAAAG
GCTGTGCCGAGGGCTGGCCAGTGAGGCTCGGCCCGGGGAAAGTGAAAGTTTGCCTGGGTCCTCTCGGCGCCAGAGCCGCTCTCCGCA
TCCCAGGACAGCGGTGCGGCCCTCGGCCGGGGCGCCCACTCCGCAGCAGCCAGCGAGCGAGCGAGCGAGCGAGGGCGGCCGACGCGC
CCGGCCGGGACCCAGCTGCCCGTATGACCGCGCCGGGCGCCGCCGGGCGCTGCCCTCCCACGGTAAGCGACGGCCGCGGCGCTGGGC
CCGGGACGGGCTGGGGCGGGGGCTCGGCGGCCAGGCGGCCGGGAGCGGCCCCTCGGAGCCGACAGCCTGGGCGCGCCGGCTGCACTG
GCCCGGCGTTCCCGCCGCGGACGAACCGCCTTTGCGCACGGACTGGGCCCTGGCGCCAGGGCGGAGGCCGGTGGCGGCCCTGCAGCT
GCACCGGGACTGCCAGGCTTCCCTGGGAAACGGAGGGCTGCTCACCCCATTGCACGGAGGGGAACACTGAGCCACCTGCAGGCAGGA
GCCCCCGCTCAGCCAGGGTGCCCTGCCTCTCTCCCTANNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNGCTGC
TTGGGGCTAGCTCAGAATCTCTTTTTCTTGCAAATTCTGGTTTCATCGGTTGGGGAGATTCTGGGCTTTGTGGTGGTTTATGGTGGT
TTGCAGTGCTTGAGGAAAATGGAAAGAGGGTTTGGTGTTGCAGAGCTGTGTCCAGGGGCAGGGTCTGCTCCTGACTGCGTAAGGGAC
GCCCTGCCCCAACTGTGGCGGTAGCAGATGCAGCCATAGAAAGTGAGGGTGGCACTGTTCATGACCAGTCAGTGGGCTCATAATAGA
TTTGCTTCTGAGGAGTCCTGGAAGAAAATGAGAGGGGAAACCTGGGGGGAAAAGAGCAGAAAATTATTTGGGGGTAGAGGAGAGCGG
GCTGCAGGTGAGGTGAGAGAGAAAGGGAAGAGTGGGTCCACAGAAAGAAAGTGCCTCACTGAGAGAGAGGAGATAGAGTTGAGAGGG
GAAGGGAGAAGGGTTAGCCATAGGCAGAAAGCAAAGCTTCGGGGCTGAAGGGGAGACCATGAGGAAAGAAGAGAAAGACAAGGGGGC
TGTGTTTAGACAGGGGAAGTCCAAGGGCCCGGTTCTGCCAGGTCTCTCCAGAGGCCTTGTTGAGGGGGAAGATGAAATGGAATAGGA
TAGGTGGCAGAGGAGGCTGGACAGCTGGACTGAGGTGTCCTAGCATCTCTGAGCCTGCAGCAGAGATTCCTTCTGAGGGCTGCTCCC
CAGGCAGAAGCCAGGGCTAGGGGTGGGAGGAAGAGGCAGTCAGAGGAGAGCCTGCCCAGCCAGATGCAAGCCAGAGTGTTAACCCCC
CACTGTCTGTCCAGATCCTCCTGAGGCAGAATGGAGGCCCCAGGTCCAGGGGTATCCCCTTTGGCCAGCTTCGAAGTCAGCCTCTCA
TGTAGTGGCTGCACTTTCCCCACACTGATGCCTCAAATGTCATCAAAGCCAGACAGCCCTTGGGGTTAAGCATTGCTCCCACCTTGC
TCTCCTCCAGGGAGGTTGTCTTCCTGAGCGGTCCCTTTCATCCATACCATTCACTCCAGACCTTGAGTGGCAGCCAGTCTGGGGTAG
ATACACCGTCTAGTGGCCCAGTTTCCTCCAATCATTCCTGARCCTAAGGTTTCTGAGTACCTGGGCCATTATTCCCACCCAGGGCAA
CCTCCCCAACAGAGCCTGAAGTCCCCCCAGCCCCCGTGACTGCCGTCCCCCTCTTCCACACTTTCCCTCCCTCAGATCAGCCAGGAT
CCTCACAGTCCCTGTGCTGAGTGGTATGAAACTATCCTTTGGCTCTTCCTTCCATGGCTCCCACCCCTCTCCTGCCATCACCTTCTC
CCCATCTTTCAAGGCCTCACTGAGGTCCCTGCTCTCTGCAGTCTCCCTGGTCACCCAGCCAGAGCTGCCTTCTCCTCCCCAGAAGAG
CTGGCAAGCATTTGGGCTTTTGGTGATATGTAGATTAGCACATTGTTGTGGGCTGTTTCATAGCCTCGTTGTGTTTTTCCAGTGAGC
AACTTATTTGCAGTATTTGTCTCAACATCTGGATTATTGAGGCAAAACCCAGAGTCCAGGATAGTGTCAACCTTCACCCTACCCTGC
CAGCATTTCCCTGAATCTGGGGGCATCAGCCGCCAGGAGGCTTGGGAGCTCTCAGGAGGTGACCTCAAGCTGGAGAAAGAAAGCCCA
AGAGTCTGTCTGCTTTTGGCTGAAGAAGGAGAGACCTAGGGCCACGGGTAGCGATGAGGAGACCCCAGTGAAGAGAAGGAAGAGAGG
AAGGAAGGGGAAAGACAGGGCATCAGGAGCCAGTCCCATCACTCTGGGGCTGAGCAACTACTGGGCCAGGTAGGTGCTGGGAGAGAA
GAGGGGTCTCAGAAAACTTCTCTGCCATGATAAGAGGTGGCTGGCTGGAGGCAAAGCCTGATGGTGCTCAAATAGGCAGGTGCCAGG
CAAGGGATGGCCCATTACTGGCACTGGTGCCTCTGATTAATCAGCTGGGCTCCTTTTGAAACACACACCCAGGGCTCCAAGATGTCT
CTGGCAAGGGATGAGTTCCAGTGAAACTGAGTTTGATCCCACAGTTTTTCCAGTGGCAGAGCCAGATCTGCATGGAACCAGGTGGGA
GGAAATGCTGGGCCCAGGGGAGCTGTCTCTGGCTTGCCATCTGAAAGCCGGTGCATGAACATGAACTTGAGGGCAGTTCAAAAAGTG
CTTGGCTCAGTGTTGATGTTGCAGAGACAGAAAAAAATGGGTAGATGCAAGTCCTGCTCTCGAGTTTCCTGTGTCATTAAAATTTAG
GCTGGTAGGCCACCATCTGTGTCAGTATTAGTTCAATGTATAGTTAGGTTTGGCTGGCTTAATCTGGGAAGGCTTACTTGTAGAAGG
TGACATCTGGGTTGTTTTCATGGGAGAACAAGGTTGTGCTGTGGCTGCTAGAAATCCTGGGAAAGCTGGATTTGAGGGATGCTGTAT
CCTGAGGTAAGGGGCAGAGCCTGTAGCATTGTAGATATGAGGCCTTTGTTTTTCTGCGTTGAGCAGGGCATGGGGATAACTGGGGAG
AGTGAGACCTGGGGAGAAATGACACCCTCTCTGTCACAGACATGGCTGGGCTCCCTGCTGTTGTTGGTCTGTCTCCTGGCGAGCAGG
AGTATCACCGAGGAGGTGTCGGAGTACTGTAGCCACATGATTGGGAGTGGACACCTGCAGTCTCTGCAGCGGCTGGTGAGTGTGTGG
CCATGCTGTATTCTACCTTCTCCCCACTGGGGAAATGAAGGCAGGAGCCAGGGAGCAGGTCAAAGAGAGCAGTTGCAGGCAGGAAAA
TAGGGCAGTGCGGGACATTGCTTGTGGTTCCCACTAGCTCCACCAGTGATACCCTTCACTAACCTTCCCAAAGTTAGGACCTCTGGT
CTCCCCAGCTCGAAGCCCTCTCTGACTGCCCTGCAGGCAGTGGATGCTGTGGGCTTCCAGCTGCTTGCCTGGGTTAGTGATTGCCCA
GGAACATCAACCACTGATTCTGAAAAGGCTTCTGAGGTCTGCTGTCCCTCAGTGGGATGCCTCCTCTGGGAAGCTAGCCCAGGCGGC
CTGCTGTGTCCAGATGTTGCATCTAGCTCCTGGACTCTCATATGTGGCGCCAGTCTGGCATCAGAGCCCCACCCAGATTTGGAGGGG
AAGCGCTTGCCTAACTCCCAGCCTTCCACACTCACTTCCCTGGCTCTTGCCCATACCCTGAGGCTGGGATGGCCTGCTCATCTGGGC
CATGAAGGAGCCTGTATCTCATTCTAATCAGACCCATACGTGGTCCAGCCAGGCAGAATCAGGTTGCTGTCATGGCTGCTGTCCTGC
TCAGTGGGAATTTACCATTCCTCTGGCCAGCCAGGCAGGGTGGGGCATCACTGGCTAACGCCAGCTCCAGGGCCCTGAGCTTGGGGC
CTGTGGGCTGTGCCTTCCGCCTCTCTTGCCCCAGCACTACTTCTCCTGTATGTAGTTGCTGTAGCAATCCAAGGTAGACCAAGAGCC
CCAGCATTCTCTGAGGCTTAAAATCCAGAACTGCTGCTCTGGGGCTAAAGAGGGCTTTAAGGGCATCCAGCTCCAACCCCTACAGGT
GTTCAATCCCCAGAGCTTGTCCAGCCTCTGCTTGAATTCCTACCATGACAGGGTGCTCACTGCCTCCAGGGAAGATTATATCCTATA
TTCTTCTATAGACATCTCTTCCTTAAAACAAATGGGCATTTGTCAGATTTCGTGGGGTGGTAGAAGGAAAGAAGAGACTTCTTGTTC
TTCTACAGCCTTCCCCTGGGCATCTGGAAGGCACTGATCTTCTCCTAGACTTGACTCTGTCTTTCCACGTGTGGTTGGCAGGGATGA
AGTTCAAACCCCAATCCACTCAGAAGCTAAGGTCCCCGTTTTGAAGAAGGCTGAAGGCTGAGTTGAGCTGTAGGTTACCCTGCAATC
GTTGGCCTGCTCTCTCTTACAGATTGACAGTCAGATGGAGACCTCGTGCCAAATTACATTTGAGTTTGTAGACCAGGAACAGTTGGT
GAGTGATGGCTTTTTACAAAATCCATGCACCAGCCTGCATGCAACTCCCAGGGTGGGGTGTGTGGGGGAGCATGAAAGCGGCAGAAT
GCCTACTGCTGGAAAGGGTGAGAGTGTGAGGATCCATGGGTGCTCAACTCTGGGGTGCCAGGATCCAGGGCTCAAGTCCCCTGCCAT
TCCCTTCTCCTGGCCTGATACATAACAAGCGCTCACTAGGTACCAAGCACTTTGCTAATGTAGTTCTGACAGTACCACTATGTGGTA
CACAAATACAGTTTATTATCCACAGAGAGGTGAAAGGAGCATAGCTAGTAGGTGCTAGAGGCCTGATTTGAATCCAGGAAGGTTGGC
TGTAGGGCTTGAGGCAAATCAATACTTCTTCCAGGTCACAAGCTTTGCTTAAGGATGTCACTTCTCTTCACCAGGCCAACCAGCAGG
CAGGTAGGCAGTCCAGCCCTTCCTGGACTTGCCCCACACCCAACCAGCCACCAAGTGCTGCTGGTTCTGCCTCTCAGCATCTCTGGG
TTCTGTCTCCTCCATCCCCACCTTCACCCCTCAATCAGTTGCTTGGACAATTTCAAGAGCCTCCTAACTGGTGTCTCCCTGCTTTCT
AGTTCACTCCTCATCTTACCACTGGGGGAAATTTTCTGAAACGCCAACCTGCTGCCTTTACCTCCTTCTCCCAAAAACATCAGGGCC
CCCATCACTCCAGAAGAAAGCCAAGGTTGTCTAGTCTGGGTTACACCAGGTCTAGCCATTCTCCACCACACAAGCCTCTATACCACC
TCCCCATTCCCTGCTTCTTCCTGCCTCCCATCTTTGCACAAGCTCTTTCCAATGACTGTCATGCCTTTCCACCTGCCCCCCAATCAT
TATTCCACATCTATTCATCTTTATGCCTCATGCAAAACTGCCTTGATACCCAGAAGTTGCCCCTGTTTCTGAGCTTCCACACACCTG
ACCCATATGCACCTCTGTTAGGACTAGAGATCACCTCATGGTCCAAAGAATAGCCTCAGTATTTTATGCACTCACTCGCTTGCTCTA
GCACTCATGCCTGAACTAGCATTTGCTACGAACCTACTGGACTAGATGCTGGGGGTAGAGAGCTAAAGCAGAGTGGGTCTTCAAGGC
AACTACCATCTCTTTAGGGAGACAAAACACAGAGGTTATTCTTTGGACCATGAGATAGTCTTCAGCACCCCTATGTCCTGGAGGATT
TGAAGGCGCCATATGGTAAAAGATATTTGTGAAAGCAAGACAAGAGGAAGACAGAAAGAAAGGCAGGGGAAGGTTCCCTGGAGCCCA
CCTTCCCTATATACATCCATCGTGATTAAAGCCTCATATTCCAGAAACCCAGAGAACTCTCTAATCAGAATCCACATTTCCTTCACT
CTAGCCCAGTCTCCAGTGGCGCTAGATCCCAAAGAGAGAGAAAGACAGGCAAGCTGAGGGCGGCATTCTCCTAGTTGCTGGCATGGT
GTGGTCCCTCCCCTGGGGGAAGGGGGAGAGCAAGCTGCAACCCTCCATCTGCCTGGGGTTGGGGGTTCCCAGGCCACAATGGCCAGG
CCATAAGCTCCAGGTTCCTGTTTTTCAGAAAGATCCAGTGTGCTACCTTAAGAAGGCATTTCTCCTGGTACAAGACATAATGGAGGA
CACCATGCGCTTCAGAGATAACACCCCCAATGCCATCGCCATTGTGCAGCTGCAGGAACTCTCTTTGAGGCTGAAGAGCTGCTTCAC
CAAGGATTATGAAGAGCATGACAAGGTAGGAAGCCCTGAGGCCTGGAGCACTGAGTGAGGGCAGAGGGTGGCTGTGGAGGCGCCGCT
CTATCCACAGGCACAGAGTACATTCGCTCACCTCGCCTCACGCCATTGCTTGCTCACTCTCATTTATTTGTCATCCCACCAACCTTT
ACCAGGCAGGAGGCAGGAGGGATCATGCTGGGCCCTGACAATGTCATCATGAGCAACAAATACATGGTCCCACCCCATGCAGCTTAT
AGTCAAGTGAGCTTGGCAGCTGTTAAACACCATGACACCAGTAAATAGAATTACAAACTATAAGGACTGTTCATTCCAACAGCAAAA
GCAGTTATTGTGTGTCTGTTGTGTACCAGACACTGTTCTAGACACCAGGAAACAGCTGTGATGAAAACAGACAAAAATCCCCACATT
CCTGGAACTTACATTTGGGGGTGATGGTAGTGGTGGTGAGACAGTTAACAAAATATGTAAGTAAAATATACAACATGTAGCATGGCA
ATAACAGCACTGGAGAAGAGTAACCAGGGATGAGACGACCCGAAGGAAGTGGAGGAGTGAGCTCGGAACAAGAACCTGAAAGGGCTC
AAGGAGAGCAACAGGGAACTCAGACTTCCATGGAGTTGGGGAAGCAGAGACAAGAAAGGCTTCTGAGTAAGCAAATATTGATTTGAG
CTCCAAAGATGAGTAAGAATTGAAAGCAGTAAGGCTAGAAGAACATTCTGGAACAGGAAAAGCTATGCACAAAGCCCTAAGGTAAAC
AAACAGGAAAGTGCACATTCAGGGACCCAAAGGGTTATCAGCATGGCCAGAGCATGGGAAGTGAAGGAGGGGGAGGACAGAAGTCAG
GAGCCAGATCACATGTCGTTTTAGAGATGTGGGATTTTGTCTTAAGGGTGAGGGAAGCTGTTGTGAACAGGCATGTGGCTTAATCTC
AGGTTATAAGACTGACCTGGTTGCTATGTGGAGGATAGACCACAGGGGGCAGGAATGGAGGCTGGGAGACCAGCAGAAGGTGTGAGT
CCAGGCTAGACACATTGGCGGCTTGGACCAAGGTGGGACAGTGAAGGTGGAGGCAAGTGGACTGGACATGTTCCCGTGGGTTTAGGG
GCAGAATCAACAAGACTTGGTACGTGCAGTGGATGTGAGAAGCTGAGGGAAAGAAAAAGCCAAGGATGGTTTTTGTCTTGAGCACCT
GGATATTCCAATGATGCTGAAACGGGGATGATCCATGGGCAGAGCAGGTTTGAGGGAAGACAGTTCAGTCTGGGGCACAGCAGACGT
GAGGTGCTCATGAAATGGTGAGTGGAGACGTTGAGGAGGCAGCTAGGTGTGCGGGTCTTGAGTGTGACCAGCAATGCACAGATCTAT
GAGGCATGGGGGTTCGGCTTATCTGTGAAGCCATGGGACTGGACAGCACTCCCACTGTGAGAAGAGAAGCAGGCCCAGGAACCCAGC
ATTTGGAGGGCAAGCAGAGGATGGGGGACCTGGCAAGGGGCAGCCTGAAGGTGGGAGGAAAAGAGGGAGAGTGCAGAGTCGAGGCAC
TGCATTCCAAGATGGATAAGGGATGGAAATTCATAAAAAAGCGTTGAGAAATCAAGCAAGGTGAGGGCTAAAATTGTCCATTGGATT
TGGCAACGTGTCACACATGCTGGATCAGGGTGAACAGAAAGAGAGGAAGTAGAGACACCACTGTGGAAGAACTTGGATCCGAATGGG
GGCAGGAAAAGAGATGTAGCTGGAGGGAGTGTGTAATTGAGGGAGGACTTTTTGTTCTGTTTTTGTTGTCGTTGTTTTGAGACAGGG
TCTCATTCTGTCACCCAAGCTGAAGTTCAGCGGCGCAATCACGACTCACTGCAGCCCAACCTCCCAGGCTCAATTGATCCTCTGACT
TCAGTCTCCTGAGTAGCTGGGACAACAGGCACACCCCACTATGCCCAGCTATTTTTTGTAGAGATAGGGTTTCGCAATGTTGCCCAG
GCTGGTCTCAAACTCCCAGGCTCAAGCAATCTGTCTGCCTTGGCCTCCCAGAGTGCTGGGATTACAAATGTGAGTTACTGTGCCTGG
CCAGGAAAACATTTTTAATAAGAGAGGTATTAAATCTGCGGTTTTCCAACATTAAGATTCCTAAGAATCACTTGGGGAATGTGTTGA
AAATGCATATTCCTAGGACACACCCCTAGAGAGTCTGATACACTGGGTTTAAGAGTGGCTCCAGGGCCGGGCGCGGTGGTTCATGCC
TCTAATCCTGGCACTTTGGGAGGCCGAGACAGTGGATCACCTGAGGTCAGGAGTTCAAGACCAGCCTGCCCAACAGGGCGAAACCCC
CCATCTTACTAAAAATACAAAAATTAGCAGCTGGTGGTGGCGCATGCCTGTAATCCAAGCTACTCGCGAGGCTGAGGCAGTAGAATC
GCTTGACCCAGGGTGGCGGAGGTTGCAGTGAGCTGAGATCGAAACTCCATCTCAAAATAAATAAATAAATAAATAAATAAATAAATA
AATAAATAAATAAATAAAAGACTGAACCCAGGAATCTGCATCTTAAGCAGCTGTCGCACTGATGATTATGACCCCCTTGGATATTCA
CTTGTAGACATGTGTGAATGCTGAGGGGCAGGCTCCAAGAGAGAGGGTGGGTGAAGCTGGAGGAGCTAGCTCCCAGCAAGTCTCTGG
AGAAGACAGTGGGAGGGATTCAGAGCTCCTGAGGGAGAGTGAGAGTCAGACTTCATCTCAAAAAAAAAAAAAATGTTTACCAGATAG
ACGTACGAATGAGAAGACTGGGCTCAGCTCCATCACTTACCAGCTAGGTGGCAGTGACCAAGTAAACTGCCGGAGTCTCACTCTCTC
CCAGGCTGGAGTGCAGTAGTGCCATCTCAGCTCACTGCAACCTTTGCCTCCCGGGTTCAAGCAATTCTCCTGCCTCAGCCTCCCGAG
TAGCTGGGATTACAGGCACGCACCACCATGCCCAGCTAATTTTTGTCTTTCTAGCTGGGATTACAGGCACGTGCCACCATGCCTGGC
TAATTTTTGTCTTTTTAGTAAAGACAAGGTTTCACCATGTTGGTCAGGCTGATCTCCAACTCCTGACCTCGTGATCAGCCTGCCTCG
GCCTCCCAACATGCTGGGATTACAGGTGTGAGCCACCGCACCCGGCCTATCTGGTAAACATTTCTTAAGGGCTGACTGCATAGGCCA
GGGAGATGCGGCGATGCCTTCAAGGACTTCACAGCCTAGAAAAGCCCATGTTTCTTCCCTGTGAAGCTCAGTTTAAGGAAAAACTTG
GTATCTGTGATTCAGTGGCATTTAGGCCCTGGATGAAGCTGTGAGCATGCATGGCATGGCCTTGTGTGAAAGGAGAGGTGGGCCCGG
GAGTGAACCCTGAGGGATGGCAGCATTTGGATATCAGGAGGAGAAGGGGAGCTTGGTAAGGAGACAGAAAGAGCAGCTTGAAAGGTG
GGAGAAGACCCAGGATGATGCAGCTTCTCAGAGCTCCAGTGGGTGTGGGAAGCCGGGGCCCCTTGAGCAGCACAGAAGACAGGGCTG
GGACCTCCTCAGCCCTCCCTCTAAGACACACCAATGCCAGCAAAGGACCCATATGCCTGGCGGCTGCCCCGTTCCCTTCTCCACACC
ACAGCTCAATGATTTTTCTCAAACTCAACTCTTACCATGCCACTGCCCACCCCTAACACCCCTTCAGATGTTTTCCCTTTGCTCTGT
GGACAAAACTAAAAGTTCTGGTCTTGGCCCACGAGGCCCTCAAGACCTGGCCCTTGCTAACCTCTCTCCTTGTGCGTCTCTTGCCAC
GCTCCTCCTAGATGGTAGTGCTGCAGCCACACCAGCCTTCTTTTAGTCTTTGGGCTTTTCATGCATTTTCTTGCCTCAGGACTTTTG
CACGGTCTGTTCTTGCAGCCTGGACAGCTCTCTGGGATCCCATCTCTTTCCTGGTTTCTCTTGCTCATCTTTTAGTTGATTCCTTGG
GGAAATTCTCCCTGAACACTTGTTTAGTTCCCCTTGTTGCACACCATCGGATGGCCCAGTACTTATTCATAGGACTCATTGCACTTG
TAACAATGTGTTTAGTCCCTGCTGGATTGCTGGACTCACTGTCCAACTCAGCCAGGAACCACCTCTCTCTTGTTCGTTGCATGATTC
CCAGGTAATTACCATATGCCTGGCACATAAACATATGTTGAATGGGTCTGTGAATAAATGACATATCTCATAACCTCAGGACTCATT
CTGCTGCAAACCAGGGGAAAGGGGAGCAAGGAAACAAAAGGGAAAAAGAAGACAGATGTGAGAAAGGCCAAGGGAATTTGACAAATA
AGATGGAGACATGGGGCCAATGGTCATGCTCACAAAAGGGGGCCCTGATCTCCTTCCAGGCCTGCGTCCGAACTTTCTATGAGACAC
CTCTCCAGTTGCTGGAGAAGGTCAAGAATGTCTTTAATGAAACAAAGAATCTCCTTGACAAGGACTGGAATATTTTCAGCAAGAACT
GCAACAACAGCTTTGCTGAATGCTCCAGCCAAGGTAAGCATGGCAGGGGCCAGCAAGTGTGTGGGGGTGGTAGCATATGGAATGGGG
ATTGGGAGGTGAGATGTGAAGCTGGGGGGACCCCTGGGGAGGCAGCCTGGCTGCTACTCGTGTTCCCGTGTGCATGAATGTGCTTGT
TCTGTGTATATATGTGGCTTCACGTACCTCTGGTCCTGGGCACATGTCTTTTCTGACATGTGTGTGCATGCACACCTACATATGTCT
GCATGTGGCAGCGTCAAGAAGGATCATGCTGGTGCCTTGAGATTAGGGAGTGGAAATGGGAGCCTGTCTTGGGAGCCCTGTAGGTAT
GGAAGCTTTCCCCACTGGCTCTGCAGGCTGGCTGAACGTGTGGGGGGTTCTGTGGAGACAGCTGACACCCATCTGGCAGTCAGGGCC
CTTGCTCCAGGAGCTCTGCTGCGAATCACCATGATACCCTGGCTCCAGAAGTCCCCAGGATGCCTTGGCTGTTTCCTCAAGGGACAA
GGCTGGATTTGAAGTCTGAGCATCATTGGAACTCCCAGGGCTGGCTCAGCTGCATACATGTGTACACTGCGTGTCCTCGTCTCTGCG
TCTCGTGCCACACGTGCCCTCCTACATTCAACCTTGCTTCCCAAGGCAGGGTCTAGGGCCCTGCAACCTGCCAGTGGCCAAGTCCAG
TCTCGTTGCTTCAATCCCAAGTCCTCAAGCCTTGGCTGCATTCTAGCCCCAGCCTGTTCCTGCCTGTGGCTGTGGCTGTGGCTGTGG
CTGTGTGGCTCCTGGCTATGCATGAAACCAGTGTCTCTGGGGCTTGAAGTTGTCTTGTATTGGTCTGGAAGGCAACCGTTCAGCCTC
CTGACCTGACTGCTGCTCACCCCTAGCTTGGCCTGTGGCCAGTGGGAACCCCTGCATGGGCTGTTCTCTTATCTTCCTTCTCCCCAA
CCCCCAGTGTGTGCATCTCAACCCTATTCTTTGTCACTGCTCATGAGACCCTGCATACGGCACCTTCCCTGTGTCATGAGCACCCAC
TCTAGTCCCATCCTCTTCTCAGCCCCAGGGCTGAGCTAGGAGATGAGGGCCCCCCAGACTCACATTCCCCTCTTGCCCCGCTCTGGG
AAAGCTGTGCTGGAGGCTAGTGACTCTATCTCCTCCCCATCTTTCTCTCTCCTTCTCTCTGTGGTTCTTTCAGATGTGGTGACCAAG
CCTGATTGCAACTGCCTGTACCCCAAAGCCATCCCTAGCAGTGACCCGGCCTCTGTCTCCCCTCATCAGCCCCTCGCCCCCTCCATG
GCCCCTGTGGCTGGCTTGACCTGGGAGGACTCTGAGGGAACTGAGGGCAGCTCCCTCTTGCCTGGTGAGCAGCCCCTGCACACAGTG
GATCCAGGCAGTGCCAAGCAGCGGCCACCCAGGAGCACCTGCCAGAGCTTTGAGCCGCCAGAGACCCCAGTTGTCAAGGACAGCACC
ATCGGTGGCTCACCACAGCCTCGCCCCTCTGTCGGGGCCTTCAACCCCGGGATGGAGGATATTCTTGACTCTGCAATGGGCACTAAT
TGGGTCCCAGAAGAAGCCTCTGGACAGGCCAGTGAGATTCCCGTACCCCAAGGGACAGAGCTTTCCCCCTCCAGGCCAGGAGGGGGC
AGCATGCAGACAGAGCCCGCCAGACCCAGCAACTTCCTCTCAGCATCTTCTCCACTCCCTGCATCAGCAAAGGGCCAACAGCCGGCA
GATGTAACTGGTACCGCCTTGCCCAGGGTGGGCCCCGTGAGGCCCACTGGCCAGGACTGGAATCACACCCCCCAGAAGACAGACCAT
CCATCTGCCCTGCTCAGAGACCCCCCGGAGCCAGGCTCTCCCAGGATCTCATCACTGCGCCCCCAGGGCCTCAGCAACCCCTCCACC
CTCTCTGCTCAGCCACAGCTTTCCAGAAGCCACTCCTCGGGCAGCGTGCTGCCCCTTGGGGAGCTGGAGGGCAGGAGGAGCACCAGG
GATCGGAGGAGCCCCGCAGAGCCAGAAGGAGGACCAGCAAGTGAAGGGGCAGCCAGGCCCCTGCCCCGTTTTAACTCCGTTCCTTTG
ACTGACACAGGCCATGAGAGGCAGTCCGAGGGATCCTCCAGCCCGCAGCTCCAGGAGTCTGTCTTCCACCTGCTGGTGCCCAGTGTC
ATCCTGGTCTTGCTGGCCGTCGGAGGCCTCTTGTTCTACAGGTGGAGGCGGCGGGTGAGTAGATCCCCATGAGGAAGAAGAGCACGT
CCCTTAGGGCAGGGGCAGAGCCTGGCGGGGGTGCAGGTGGGGGGACAGCTTGGGTGCGGCCTGAGTTCTTCAGACACAGAGAGATAG
GGGCTGGCTCAGCACAGAGGATGAGAGGTGGAAATGGAGGATTACTTCGAGATTGGGAAGGTTCAAGCTATAAGGTCAATGGGAAAG
GGACTGGAGCAGAAGGGAGCGGGAGAGAATATTCATGGGCTGACAGCAGGATGATATCCAGACGGGCAGGGAGCTGGAGGAGGAGAG
CAATGCTGTGTGAGCGTGTCAGGGCAGGCGTACACTCGAAGCTCTGCAGAGCCTGGGGCAGGAGGACCCATAGGGAACAGCCTGCAA
GGGTGTGGGGAGAGGAGAGGGGACACCATCAGCAGAGAGACCTCACAAATCTCCCTTGGGCCTCTGGCTGATCCCCACTTTTGGGAA
GTTGGGAGGACTCTTGCAACTCTCTCATAAATACCTGGGGCAGCCCTTACTGGGGATGGGCCACCGCCCCCCCACACTCCCCCAGCT
CCTCAGTGAGATGCTCTTTCCTGTCCTCAGAGCCATCAAGAGCCTCAGAGAGCGGATTCTCCCTTGGAGCAACCAGAGGGCAGGTGA
GAGCTTGAGGTGGGGCTCTGGGAGGCACTGGGGGCCTGGCTTGGGATCTGTTTCCCCTCTTCGTGGTGGTGGGGCTCTCCTGCTTGC
TCTGAGGAAATGGAGCTGCAGAACAGGATGGGGGAGAGAAGAAGGGCCTCTGTCCTTTCCCAGATATCTGGGCTTTTTCCTGATTTC
TCCGTAGAGTTAGACAGTTCTGGGTCTTTTCAATCTGACAGGGCCTAGAGCATGTCTGGGGCTTAGGGGTAAACTTACGGAGTCCCC
TTATTCCCCTGCTCCTGCTGATGTCTAATGCCAGCCCTGTGCTCTGCCTGCAGCCCCCTGACTCAGGATGACAGACAGGTGGAACTG
CCAGTGTAGAGGGAATTCTAAGGTAAGATTCTGACTTTGATCTCCACTCCTAAAACTTACCTATACCCTTTTCCAGTCACGTTCACC
TGTTGGTGACACCAATTCCCCTGAGGCCCCCACACTGACTCCCATGCCTCTCTCCAGTTCCCTTTCCATACATCGCCAGCCAGGTCC
AGCCACCCTCTCCTTTCCAGGTCCTCAGCTATCCCCCTGCCCCTCCATTCCATCCACCCCCGCTGAGGCCAGGGACTATTACCTCTG
CATTGGGCTTCTAGTGTTGCTGCCACCTGTGACCTCAGAGCAACTAAACCCCCCTTTTTCAAATGTGGCCTTTATAAGGTCTTTACC
CTCCTGGAGAATCTTCAGTAGCTCTGTGTGGCAGCCACTGCAAGCCCTATATTCTTGGCTTGGCTGCCAAGCCCCTTCGACAGCAGC
TGCTTGCCAGATCGCCACCCCCGCACCAGTTAACCCTCCTCCCCAGCTGAGGTCAGTTCTGCTTCCTTGCTTTTAGGATCATGTTGA
CATAGAACCTGAGTCTTGGAAGGGCTCTCAGCGGTCATTTGGCCCTGGTTCCCACCCCAAAGGGACTGCCCCTCTCCATGTCCCCAA
CAGGGAGCCAGCCAGCTGGCTGTCACACACTCCCAGATCCAGCCCAGCTCCCTGCCTAGCCACAGCCCATGCCATCTTCCAGTCAGA
CTGGGAGAAACGTCTTCCTCATGCGAGGCAAAACTGGCTTCCCTGCCCCCTCCATACTGCTCCTCCCTCCTGTTCTCATGAAAACCC
TCAGATATTTCAAGTCAGGTATCCATGTCCCCTTTGAGAATTCTCTAAGCTAAGTATGGCCAAGTCCCTCAAACGTTCCTTATATGA
TATGGTTTTCAGACCCCTCACCATCCTGGACACACTCGTTTGTCAATGTCCCTCTGAAAATGTGACGCCCAGCCCCGGACACAGTAC
TCCAGATGTTGTCTGACCAGCTCAGAGAGAGTACAGTGGGACTGTTACCTTCCTTGATATGGACAGTATTCTTCTATTTGTGCAGAT
TAAGATTGCATTAGTTTTTTTCTTAACAACTGCATCATACTGTTGTCATATGTTGAGCCTGTGGTCTATTAAAACCCCTAGTTCCAT
TTCCCATAAACTTCTGTCAAGCCAGACCATCTCTACCCTGTACTTGGACAACTTAACTTTTTTAACCAAAGTGCAGTTTATGTTCAC
CTTTGTTAAAGCCACCTTGTTGGTTTCTGCCCATCACCTGAACCTACTGAAGTTGTGTGAAATCCTAATTCTGTCATCTCCGTAGCC
CTCCCAGTTGTGCCTCCTGCACATTGATGAGTGCCTGCTGTTGTCTTTGCCCATGTTGTTGATGTAGCTGTGACCCTATTGTTCCTC
ACCCCTGCCCCCCGCCAACCCCAGCTGGCCCACCTCTTCCCCCTCCCACCCAAGCCCACAGCCAGCCCATCAGGAAGCCTTCCTGGC
TTCTCCACAACCTTCTGACTGCTCTTTTCAGTCATGCCCCTCCTGCTCTTTTGTATTTGGCTAATAGTATATCAATTTGCACTTATT
TGGATGTTGTTTTCATATTTTTTAAAATCTTATTTTATATGTATTGTACATTCCACATCCCTAAGATTGTAAGCCCCTTGAGGGCAG
GGACTGTCTTCCACTTTTTTTTGTGTTTCTTTTTCCATCCTGGGTTCTACTAAGAGAGGGCCTCTCCACATTTGCCAAACATGCAAT
AAATGTTGACTGACTGGCTTCAGGGCTGACTCACGGACTGACCCTGGCCAGCTGTCTCTAACTGACCAGCCTCTAGCTGACCAGTTA
ACCAGCTAACTGACTGACTCTGACTGACTGGCTGACCAGCTGGCTGGGTGAATGACTAACCAACCAACTATCTTGGTAACTGCCTAA
CTGACCAGCTGTTGCTTGGGGAGCCCATTGAGCAACTGACTAGCTGATTGCCTGACTAATTGGCTGGTTGGCTGACTAGCTGGGGTG
ACCCAGTCAAGTTTCCAGTCACATCCTTTACCAAGTGCAAGCCAGTGCCGCAGGGCTCTCCCATGTGTGGAGTGACAGGTCTGGGCC
CCTTCTAGAGAGATAGGGAATTTGGCAAATGCTCAAAACCAGGGGAACCTATTTAGACAAGAACCAGCAGTAACAACATATCTGGAA
TCCTGGGGTCTACCTTGTCAGTGGGGTGGGCTAAGATGGGTGAGTGGGTCAGCTGGGGCACTGGCAAGACTGTGAAATGACCCAGGC
CTCCTCAGAACTGGTTGCTGGTGGCTCAGTGCCACAACACCCTTCCCACTGGTCGTCTGTCCCCAAACCCCTCGCCGTGGTCTTGGC
CCTCTGTCCCACCCCTCTACACGCCCACCCTCCTTTCCCCACTTGCTTATTCTCAGGCCCGTCCCAAGCTTTCCTCTTCCCCCTCAA
GATTTCACTTGGAGAGGGAATCCAGGGCAGGGGCTACATCCAGATCCCACTTGGCCCGGCAGGTCCAGCCCTAAGGTGGTCTCCAGG
CCAGGCACCAGTGGCCTTGGAGAGATTGAGTGCCAGCCCCTAGAGTGACTGCTGCTGTGATTTGAGAAGTGAGAGAGAGAAACATAG
AGGCAGAGACTGAGATAGGGACTCAGAGCCACAGAAAATGCAGACACCAGCACCTCAGCCCTAAGCAGCTAGAAGATGGGGCAGCAG
GCTGGAACTCGCTCACTGGGTCCTTCTCCCCTCCTGGGCCCTAGCTGGGCCTGTGGCCTGGCTGTACCCAGCATGGCTCATGTCCAG
TTTATGGTTCCCCTTCTTTCCCTCTAGAGCATGGGAATGGGCAGGGAGCCAGAGGCTGTGGTTCCATCTTCCCTCCTTACCCAGTAG
GGCCTATGGATGTTAGGGAGGGAGGCTCCACACAAGGTCCAAAGTCAAACTGTTGGATGGGGTCCAGGGGATTTCTGCTCTGGGAAC
AAAGCGTCCTGAGGATGCTGGGCAAGAGGACAAGGATGGGGGTGGAAGGCGAGAGGACAAGGCCCTGACTCCCATGGATGATGCGGG
AGGCAAGGCCCTCGGCCCATCCAGGAGGGGCTGGGTTGCTTAGGGGTCGCGTGCCAAATGACAGTGTGGGAATTTCCTTGTCCTGGC
ACTGCCTCTAGACTTTTCCTCTCTGGCCCAAGTTGTTCCTCTTCAGCTAGCTCTGCCCTACTGTCTTGAACATCATATGTGATGCCC
CTCTGATGAAATGAAAGATTCTTCTTTGCTAGTGTTAGCAGGAATCACCCTTTGTTCACTGGACCAAGGCCAATGAGATGACCTCGC
CTGAGGAGGGGTCCAAAGACATTCAAGTGCCCGCTTCGCTCCTTGTCAGAGCCCTCTGCTTGAGCAGTAACCCCTCTTCCCAGCCCT
GCCCAGACCCCTCCCGGGCCCCGTTATCTTCCACTGAGGCCCTGATTCCCCAGGAGGAGGTAGGAGGTGAGGCCAGTTGGGCCAGAG
GCCTGGGCTGGTGCCAGAGAAAGCTGCCTGGTCTCTCTGTGGTGTCTGGAAAGGCCAGGGAAGGGAGGAAAGGAAGTCCTGCCTGCA
GCACCGCCAGCAGCCCACAGGAATGCCACACCTCCCAATCCATGTCCTCAACGACCCTCCCCGCATTCCCACTCTGCTGACTAGCCT
TTCCTTCTCTCCCACAGCTGGACGCACAGAACAGTCTCTCCGTGGGAGGAGACATTATGGGGCGTCCACCACCACCCCTCCCTGGCC
ATCCTCCTGGAATGTGGTCTGCCCTCCACCAGAGCTCCTGCCTGCCAGGACTGGACCAGAGCAGCCAGGCTGGGGCCCCTCTGTCTC
AACCCGCAGACCCTTGACTGAATGAGAGAGGCCAGAGGATGCTCCCCATGCTGCCACTATTTATTGTGAGCCCTGGAGGCTCCCATG
TGCTTGAGGAAGGCTGGTGAGCCCGGCTCAGGACCCTCTTCCCTCAGGGGCTGCACCCTCCTCTCACTCCCTTCCATGCCGGAACCC
AGGCCAGGGACCCACCGGCCTGTGGTTTGTGGGAAAGCAGGGTGGACGCTGAGGAGTGAAAGAACCCTGCACCCAGAGGGCCTGCCT
GGTGCCAAGGTATCCCAGCCTGGACAGGCATGGACCTGTCTCCAGAGAGAGGAGCCTGAAGTTCGTGGGGCGGGACAGCGTCGGCCT
GATTTCCCGTAAAGGTGTGCAGCCTGAGAGACGGGAAGAGGAGGCCTCTGGACCTGCTGGTCTGCACTGACAGCCTGAAGGGTCTAC
ACCCTCGGCTCACCTAAGTGCCCTGTGCTGGTTGCCAGGCGCAGAGGGGAGGCCAGCCCTGCCCTCAGGACCTGCCTGACCTGCCAG
TGATGCCAAGAGCGGGATCAAGCACTGGCCTCTGCCCCTCCTCCTTCCAGCACCTGCCAGAGCTTCTCCAGGAGGCCAAGCAGAGGC
TCCCCTCATGAAGGAAGCCATTGCACTGTGAACACTGTACCTGCCTGCTGAACAGCCTGCCCCCGTCCATCCATGAGCCAGCATCCG
TCCGTCCTCCACTCTCCAGCCTCTCCCCAGCCTCCTGCACTGAGCTGGCCTCACCAGTCGACTGAGGGAGCCCCTCAGCCCTGACCT
TCTCCTGACCTGGCCTTTGACTCCCCGGAGTGGAGTGGGGTGGGAGAACCTCCTGGGCCGCCAGCCAGAGCCGGTCTTTAGGCTGTG
TTGTTCGCCAGGTTTCTGCATCTTGCACTTTGACATTCCCAAGAGGGAAGGGACTAGTGGGAGAGAGCAAGGGAGGGGAAGGGCACA
GACAGAGAGGCTACAGGGCGAGCTCTGACTGAACATGGGCCTTTGAAATATAGGTATGCACCTGAGGTTGGGGGAGGGTCTGCACTC
CCAAACCCCAGCGCAGTGTCCTTTCCCTGCTGCCGACAGGAACCTGGGGCTGAGCAGGTTATCCCTGTCAGGAGCCCTGGACTGGGC
TGCATCTCAGCCCCACCTGCATGGTATCCAGCTCCCATCCACTTCTCACCCTTCTTTCCTCCTGACCTTGGTCAGCAGTGATGACCT
CCACTCTCACCCACCCCCTCTACCATACCTCTAACCAGGCAAGCCAGGGTGGGAGAGCAATCAGGAGAGCCAGGCCTCAAAGCTTCC
AATGCCTGGAGGGCCTCCACTTTGTGGCCAGCCTGTGGTGGTGGCTCTGAGGCCTAGGCAACGAGCGACAGGGCTGCCAGTTGCCCC
TGGGTTCCTTTGTGCTGCTGTGTGCCTCCTCTCCTGCCGCCCTTTGTCCTCCGCTAAGAGACCCTGCCCTACCTGGCCGCTGGGCCC
CGTGACTTTCCCTTCCTGCCCAGGAAAGTGAGGGTCGGCTGGCCCCACCTTCCCTGTCCTGATGCCGACAGCTTAGGGAAGGGCAGT
GAACTTGCATATGGGGCTTAGCCTTCTAGTCACAGCCTCTATATTTGATGCTAGAAAACACATATTTTTAAATGGAAGAAAAATAAA
AAGGCATTCCCCCTTCATCCCCCTACCTTAAACATATAATATTTTAAAGGTCAAAAAAGCAATCCAACCCACTGCAGAAGCTCTTTT
TGAGCACTTGGTGGCATCAGAGCAGGAGGAGCCCCAGAGCCACCTCTGGTGTCCCCCCAGGCTACCTGCTCAGGAACCCCTTCTGTT
CTCTGAGAAGTCAAGAGAGGACATTGGCTCACGCACTGTGAGATTTTGTTTTTATACTTGGAAGTGGTGAATTATTTTATATAAAGT
CATTTAAATATCTATTTAAAAGATAGGAAGCTGCTTATATATTTAATAATAAAAGAAGTGCACAAGCTGCCATGTGAGT
HUMAN mRNA SEQUENCE: hR27-014.1 (Seq ID No: 41)
CCTGGGTCCTCTCGGCGCCAGAGCCGCTCTCCGCATCCCAGGACAGCGGTGCGGCCCTCGGCCGGGGCGCCCACTCCGCAGCAGCCA
GCGAGCGACTGCCCGTATGACCGCGCCGGGCGCCGCCGGGCGCTGCCCTCCCACGACATGGCTGGGCTCCCTGCTGTTGTTGGTCTG
TCTCCTGGCGAGCAGGAGTATCACCGAGGAGGTGTCGGAGTACTGTAGCCACATGATTGGGAGTGGACACCTGCAGTCTCTGCAGCG
GCTGATTGACAGTCAGATGGAGACCTCGTGCCAAATTACATTTGAGTTTGTAGACCAGGAACAGTTGAAAGATCCAGTGTGCTACCT
TAAGAAGGCATTTCTCCTGGTACAAGACATAATGGAGGACACCATGCGCTTCAGAGATAACACCCCCAATGCCATCGCCATTGTGCA
GCTGCAGGAACTCTCTTTGAGGCTGAAGAGCTGCTTCACCAAGGATTATGAAGAGCATGACAAGGCCTGCGTCCGAACTTTCTATGA
GACACCTCTCCAGTTGCTGGAGAAGGTCAAGAATGTCTTTAATGAAACAAAGAATCTCCTTGACAAGGACTGGAATATTTTCAGCAA
GACTGCAACAACAGCTTTGCTGAATGCTCCAGCCAAGATGTGGTGACCAAGCCTGATTGCAACTGCCTGTACCCCAAAGCCAATCCC
TAGCAGTGACCCGGCCTCTGTCTCCCCTCATCAGCCCCTCGCCCCCTCCATGGCCCCTGTGGCTGGCTTGACCTGGGAGGACTCTGA
GGGAACTGAGGGCAGCTCCCTCTTGCCTGGTGAGCAGCCCCTGCACACAGTGGATCCAGGCAGTGCCAAGCAGCGGCCACCCAGGAG
CACCTGCCAGAGCTTTGAGCCGCCAGAGACCCCAGTTGTCAAGGACAGCACCATCGGTGGCTCACCACAGCCTCGCCCCTCTGTCGG
GGCCTTCAACCCCGGGATGGAGGATATTCTTGACTCTGCAATGGGCACTAATTGGGTCCCAGAAGAAGCCTCTGGAGAGGCCAGTGA
GATTCCCGTACCCCAAGGGACAGAGCTTTCCCCCTCCAGGCCAGGAGGGGGCAGCATGCAGACAGAGCCCGCCAGACCCAGCAACTT
CCTCTCAGCATCTTCTCCACTCCCTGCATCAGCAAAGGGCCAACAGCCGGCAGATGTAACTGGTACCGCCTTGCCCAGGGTGGGCCC
CGTGAGGCCCACTGGCCAGGACTGGAATCACACCCCCCAGAAGACAGACCATCCATCTGCCCTGCTCAGAGACCCCCCGGAGCCAGG
CTCTCCCAGGATCTCATCACTGCGCCCCCAGGGCCTCAGCAACCCCTCCACCCTCTCTGCTCAGCCACAGCTTTCCAGAAGCCACTC
CTCGGGCAGCGTGCTGCCCCTTGGGGAGCTGGAGGGCAGGAGGAGCACCAGGGATCGGAGGAGCCCCGCAGAGCCAGAAGGAGGACC
AGCAAGTGAAGGGGCAGCCAGGCCCCTGCCCCGTTTTAACTCCGTTCCTTTGACTGACACAGGCCATGAGAGGCAGTCCGAGGGATC
CTCCAGCCCGCAGCTCCAGGAGTCTGTCTTCCACCTGCTGGTGCCCAGTGTCATCCTGGTCTTGCTGGCCGTCGGAGGCCTCTTGTT
CTACAGGTGGAGGCGGCGGAGCCATCAAGAGCCTCAGAGAGCGGATTCTCCCTTGGAGCAACCACAGGGCAGCCCCCTGACTCAGGA
TGACAGACAGGTGGAACTGCCAGTGTAGAGGGAATTCTAAGCTGGACGCACAGAACAGTCTCTCCGTGGGAGGAGACATTATGGGGC
GTCCACCACCACCCCTCCCTGGCCATCCTCCTGGAATGTGGTCTGCCCTCCACCAGAGCTCCTGCCTGCCAGGACTGGACCAGAGCA
GCCAGGCTGGGGCCCCTCTGTCTCAACCCGCAGACCCTTGACTGAATGAGAGAGGCCAGAGGATGCTCCCCATGCTGCCACTATTTA
TTGTGAGCCCTGGAGGCTCCCATGTGCTTGAGGAAGGCTCGTGAGCCCGGCTCAGGACCCTCTTCCCTCAGGGGCTGCACCCTCCTC
TCACTCCCTTCCATGCCGGAACCCAGGCCAGGGACCCACCGGCCTGTGGTTTGTGGGAAAGCAGGGTGGACGCTGACGAGTGAAAGA
ACCCTGCACCCAGAGGGCCTGCCTGGTGCCAAGGTATCCCAGCCTGGACAGGCATGGACCTGTCTCCAGAGAGAGGAGCCTGAAGTT
CGTGGGGCGGGACAGCGTCGGCCTGATTTCCCGTAAAGGTGTGCAGCCTGAGAGACGGGAAGAGGAGGCCTCTGGACCTGCTGGTCT
GCACTGACAGCCTGAAGGGTCTACACCCTCGGCTCACCTAAGTGCCCTGTGCTGGTTGCCAGGCGCAGAGGGGAGGCCAGCCCTGCC
CTCAGGACCTGCCTGACCTGCCAGTGATGCCAAGAGGGGGATCAAGCACTGGCCTCTGCCCCTCCTCCTTCCAGCACCTGCCAGAGC
TTCTCCAGGAGGCCAAGCAGAGGCTCCCCTCATGAAGGAAGCCATTGCACTGTGAACACTGTACCTGCCTGCTGAACAGCCTGCCCC
CGTCCATCCATGAGCCAGCATCCGTCCGTCCTCCACTCTCCAGCCTCTCCCCAGCCTCCTGCACTGAGCTGGCCTCACCAGTCGACT
GAGGGAGCCCCTCAGCCCTGACCTTCTCCTGACCTGGCCTTTGACTCCCCGGAGTGGAGTGGGGTGGGAGAACCTCCTGGGCCGCCA
GCCAGAGCCGGTCTTTAGGCTGTGTTGTTCGCCCAGGTTTCTGCATCTTGCACTTTGACATTCCCAAGAGGGAAGGGACTAGTGGGA
GAGAGCAAGGGAGGGGAGGGCACAGACAGAGAGGCTACAGGGCGAGCTCTGACTGAAGATGGGCCTTTGAAATATAGGTATGCACCT
GAGGTTGGGGGAGGGTCTGCACTCCCAAACCCCAGCGCAGTGTCCTTTCCCTGCTGCCGACAGGAACCTGGGGCTGAGCAGGTTATC
CCTGTCAGGAGCCCTGGACTGGGCTGCATCTCAGCCCCACCTGCATGGTATCCAGCTCCCATCCACTTCTCACCCTTCTTTCCTCCT
GACCTTGGTCAGCAGTGATGACCTCCAACTCTCACCCACCCCCTCTACCATCACCTCTAACCAGGCAAGCCAGGGTGGGAOAGCAAT
CAGGAGAGCCAGGCCTCAGCTTCCAATGCCTGGAGGGCCTCCACTTTGTGGCCAGCCTGTGGTGGTGGCTCTGAGGCCTAGGCAACG
AGCGACAGGGCTGCCAGTTGCCCCTGGGTTCCTTTGTGCTGCTGTGTGCCTCCTCTCCTGCCGCCCTTTGTCCTCCGCTAAGAGACC
CTGCCCTACCTGGCCGCTGGGCCCCGTGACTTTCCCTTCCTGCCCAGGAAAGTGAGGGTCGGCTGGCCCCACCTTCCCTGTCCTGAT
GCCGACAGCTTAGGGAAGGGCAGTGAACTTGCATATGGGGCTTAGCCTTCTAGTCACAGCCTCTATATTTGATGCTAGAAAACACAT
ATTTTTAAATGGAAGAAAAATAAAAAGGCATTCCCCCTTCATCCCCCTACCTTAAACATATAATATTTTAAAGGTCAAAAAAGCAAT
CCAACCCACTGCAGAAGCTCTTTTTGAGCACTTGGTCGCATCAGAGCAGGAGGAGCCCCAGAGCCACCTCTGGTGTCCCCCCAGGCT
ACCTGCTCAGGAACCCCTTCTGTTCTCTGAGAAGTCAAGAGAGGACATTGGCTCACGCACTGTGAGATTTTGTTTTTATACTTGGAA
GTGGTGAATTATTTTATATAAAGTCATTTAAATATCTATTTAAAAGATAGGAAGCTGCTTATATATTTAATAATAAAAGAAGTGCAC
AAGCTGCCATGTGAGT
HUMAN PROTEIN SEQUENCE: hP27-014.1 (Seq ID No: 42)
MTAPGAAGRCPPTTWLGSLLLLVCLLASRSITEEVSEYCSHMIGSGHLQSLQRLIDSQMETSCQITFEFVDQEQLKDPVCYLKKAFL
LVQDIMEDTMRFRDNTPNAIAIVQLQELSLRLKSCFTKDYEEHDKACVRTFYETPLQLLEKVKNVENETKNLLDKDWNIFSKNCNNS
FAECSSQDVVTKPDCNCLYPKAIPSSDPASVSPHQPLAPSMAPVAGLTWEDSEGTEGSSLLPGEQPLHTVDPGSAKQRPPRSTCQSF
EPPETPVVKDSTIGGSPQPRPSVGAFNPGMEDILDSANGTNWVPEEASGEASEIPVPQGTELSPSRPGGGSMQTEPARPSNFLSASS
PLPASAKGQQPADVTGTALPRVGPVRPTGQDWNHTPQKTDHPSALLRDPPEPGSPRISSLRPQGLSNPSTLSAQPQLSRSHSSGSVL
PLGELEGRRSTRDRRSPAEPEGGPASEGAARPLPRFNSVPLTNTGHERQSEGSSSPQLQESVFHLLVPSVILVLLAVGGLLFYRWRR
RSHQEPQRADSPLEQPEGSPLTQDDRQVELPV*
HUMAN mRNA SEQUENCE: hR27-014.2 (Seq ID No: 43)
TTGGGACGATCATAGAGCGCTAGCACTGAATCAGCCTGGAGAGCGCGGAAGGAAAGGGTCGGTCCGCAGAGGGCGCGGGGAAGGCAG
GGTGGGGACGCGGTGGAGCCCGCGCTCGTTTGCTGAAGGCTTGGAAGTGCAGCGCAGAAGACAGAGGGTGACTAGGAAGACGCGCGA
GCGGGGCTGGCCGGCCGGCGGGTGGGGGAGGGGAGGCGGGGGAAGGCGGCTGAGTGGGCCTCTGGAGTGTGTGTGTCTGTGTCAGTG
TGTGTGTGTGTGTGTGTATGTGTGTGTCTGGCGCCTGGCCAGGGTGATTTCCCATAAACCACATGCCCCCCAGTCCTCTCTTAAAAG
GCTGTGCCGAGGGCTGGCCAGTGAGGCTCGGCCCGGGGAAAGTGAAAGTTTGCCTGGGTCCTCTCCGCGCCAGAGCCGCTCTCCGCA
TCCCAGGACAGCGGTGCGGCCCTCGGCCGGGGCGCCCACTCCGCAGCAGCCAGCGAGCGAGCGAGCGAGCGAGGGCGGCCGACGCGC
CCGGCCGGGACCCAGCTGCCCGTATGACCGCGCCGGGCGCCGCCGGGCGCTGCCCTCCCACGACATGGCTGGGCTCCCTGCTGTTGT
TGGTCTGTCTCCTGGCGAGCAGGAGTATCACCGAGGAGGTGTCGGAGTACTGTAGCCACATGATTGGGAGTGGACACCTGCAGTCTC
TGCAGCGGCTGATTGACAGTCAGATGGAGACCTCGTGCCAAATTACATTTGAGTTTGTAGACCAGGAACAGTTGAAAGATCCAGTGT
GCTACCTTAAGAAGGCATTTCTCCTGGTACAAGACATAATGGAGGACACCATGCGCTTCAGAGATAACACCCCCAATGCCATCGCCA
TTGTGCAGCTGCAGGAACTCTCTTTGAGGCTGAAGAGCTGCTTCACCAAGGATTATGAAGAGCATGACAAGGCCTGCGTCCGAACTT
TCTATGAGACACCTCTCCAGTTGCTGGAGAAGGTCAAGAATGTCTTTAATGAAACAAAGAATCTCCTTGACAAGGACTGGAATATTT
TCAGCAAGAACTGCAACAACAGCTTTGCTGAATGCTCCAGCCAAGATGTGGTGACCAAGCCTGATTGCAACTGCCTGTACCCCAAAG
CCATCCCTAGCAGTGACCCGGCCTCTGTCTCCCCTCATCAGCCCCTCGCCCCCTCCATGGCCCCTGTGGCTGGCTTGACCTGGGAGG
ACTCTGAGGGAACTGAGGGCAGCTCCCTCTTGCCTGCTGAGCAGCCCCTGCACACAGTGGATCCAGGCAGTGCCAAGCAGCGGCCAC
CCAGGAGCACCTGCCAGAGCTTTGAGCCGCCAGAGACCCCAGTTGTCAAGGACAGCACCATCGGTGGCTCACCACAGCCTCGCCCCT
CTGTCGGGGCCTTCAACCCCGGGATGGAGGATATTCTTGACTCTGCAATGGGCACTAATTGGGTCCCAGAAGAAGCCTCTGGAGAGG
CCAGTGAGATTCCCGTACCCCAAGGGACAGAGCTTTCCCCCTCCAGGCCAGGAGGGGGCAGCATGCAGACAGAGCCCGCCAGACCCA
GCAACTTCCTCTCAGCATCTTCTCCACTCCCTGCATCAGCAAAGGGCCAACAGCCGGCAGATGTAACTGGCCATGAGAGGCAGTCCG
AGGGATCCTCCAGCCCGCAGCTCCAGGAGTCTGTCTTCCACCTGCTGGTGCCCAGTGTCATCCTGGTCTTGCTGGCCGTCGGAGGCC
TCTTGTTCTACAGGTGGAGGCGGCGGAGCCATCAAGAGCCTCAGAGAGCGGATTCTCCCTTGGAGCAACCAGAGGGCAGCCCCCTGA
CTCAGGATGACAGACAGGTGGAACTGCCAGTGTAGAGGGAATTCTAAGACCCCTCACCATCCTGGACACACTCGTTTGTCAATGTCC
CTCTGAAAATGTGACGCCCAGCCCCGGACACAGTACTCCAGATGTTGTCTGACCAGCTCAGAGAGAGTACAGTGGGACTGTTACCTT
CCTTGATATGGACAGTATTCTTCTATTTGTGCAGATTAAGATTGCATTAGTTTTTTTCTTAACAACTGCATCATACTGTTGTCATAT
GTTGAGCCTGTGGTCTATTAAAACCCCTAGTTCCATTTCCCATAAACTTCTGTCAAGCCAGACCATCTCTACCCTGTACTTGGACAA
CTTAACTTTTTTAACCAAAGTGCAGTTTATGTTCACCTTTGTTAAAGCCACCTTGTTGGTTTCTGCCCATCACCTGAACCTACTGAA
GTTGTGTGAAATCCTAATTCTGTCATCTCCGTAGCCCTCCCAGTTGTGCCTCCTGCACATTGATGAGTGCCTGCTGTTGTCTTTGCC
CATGTTGTTGATGTAGCTGTGACCCTATTGTTCCTCACCCCTGCCCCCCGCCAACCCCAGCTGGCCCACCTCTTCCCCCTCCCACCC
AAGCCCACAGCCAGCCCATCAGGAAGCCTTCCTGGCTTCTCCACAACCTTCTGACTGCTCTTTTCAGTCATGCCCCTCCTGCTCTTT
TGTATTTGGCTAATAGTATATCAATTTGCACTTATCTGGACGCACAGAACAGTCTCTCCGTGGCAGGAGACATTATCGGGCGTCCAC
CACCACCCCTCCCTGGCCATCCTCCTGGAATGTGGTCTGCCCTCCACCAGAGCTCCTGCCTGCCAGGACTGGACCAGAGCAGCCAGG
CTGGGGCCCCTCTGTCTCAACCCGCAGACCCTTGACTGAATGAGAGAGGCCAGAGGATGCTCCCCATGCTGCCACTATTTATTGTGA
GCCCTGGAGGCTCCCATGTGCTTGAGGAAGGCTGGTGAGCCCGGCTCAGGACCCTCTTCCCTCAGGGGCTGCACCCTCCTCTCACTC
CCTTCCATGCCGGAACCCAGGCCAGGGACCCACCGGCCTGTGGTTTGTGGGAAAGCAGGGTGGACGCTGAGGAGTGAAAGAACCCTG
CACCCAGAGGGCCTGCCTGGTGCCAAGGTATCCCAGCCTGGACAGGCATGGACCTGTCTCCAGAGAGAGGAGCCTGAAGTTCGTGGG
GCGGGACAGCGTCGGCCTGATTTCCCGTAAAGGTGTGCAGCCTGAGAGACGGGAAGAGGAGGCCTCTGGACCTGCTGGTCTGCACTG
ACAGCCTGAAGGGTCTACACCCTCGGCTCACCTAAGTGCCCTGTGCTGGTTGCCAGGCGCAGAGGGGAGGCCAGCCCTGCCCTCAGG
ACCTGCCTGACCTGCCAGTGATGCCAAGAGGGGGATCAAGCACTGGCCTCTGCCCCTCCTCCTTCCAGCACCTGCCAGAGCTTCTCC
AGGAGGCCAAGCAGAGGCTCCCCTCATGAAGGAAGCCATTGCACTGTGAACACTGTACCTGCCTGCTGAACAGCCTGCCCCCGTCCA
TCCATGAGCCAGCATCCGTCCGTCCTCCACTCTCCAGCCTCTCCCCAGCCTCCTGCACTGAGCTGGCCTCACCAGTCGACTGAGGGA
GCCCCTCAGCCCTGACCTTCTCCTGACCTGGCCTTTGACTCCCCGGAGTGGAGTGGGGTGGGAGAACCTCCTGGGCCGCCAGCCAGA
GCCGGTCTTTAGGCTGTGTTGTTCGCCCAGGTTTCTGCATCTTGCACTTTGACATTCCCAAGAGGGAAGGGACTAGTGGGAGAGAGC
AAGGGAGGGGAGGGCACAGACAGAGAGGCTACAGGGCGAGCTCTGACTGAAGATGGGCCTTTGAAATATAGGTATGCACCTGAGGTT
GGGGGAGGGTCTGCACTCCCAAACCCCAGCGCAGTGTCCTTTCCCTGCTGCCGACAGGAACCTGGGGCTGAGCAGGTTATCCCTGTC
AGGAGCCCTGGACTGGGCTGCATCTCAGCCCCACCTGCATGGTATCCAGCTCCCATCCACTTCTCACCCTTCTTTCCTCCTGACCTT
GGTCAGCAGTGATGACCTCCAACTCTCACCCACCCCCTCTACCATCACCTCTAACCAGGCAAGCCAGGGTGGGAGAGCAATCAGGAG
AGCCAGGCCTCAGCTTCCAATGCCTGGAGGCCCTCCACTTTGTGGCCAGCCTGTGGTGGTGGCTCTGAGGCCTAGGCAACGAGCGAC
AGGGCTGCCAGTTGCCCCTGGGTTCCTTTGTGCTGCTGTGTGCCTCCTCTCCTGCCGCCCTTTGTCCTCCGCTAAGAGACCCTGCCC
TACCTGGCCGCTGGGCCCCGTGACTTTCCCTTCCTGCCCAGGAAAGTGAGGGTCGGCTGGCCCCACCTTCCCTGTCCTGATGCCGAC
AGCTTAGGGAAGGGCAGTGAACTTGCATATGGGGCTTAGCCTTCTAGTCACAGCCTCTATATTTGATGCTAGAAAACACATATTTTT
AAATGGAAGAAAAATAAAAAGGCATTCCCCCTTCATCCCCCTACCTTAAACATATAATATTTTAAAGGTCAAAAAAGCAATCCAACC
CACTGCAGAAGCTCTTTTTGAGCACTTGGTGGCATCAGAGCAGGAGGAGCCCCAGAGCCACCTCTGGTGTCCCCCCAGGCTACCTGC
TCAGGAACCCCTTCTGTTCTCTGAGAAGTCAAGAGAGGACATTGGCTCACGCACTGTGAGATTTTGTTTTTATACTTGGAAGTGGTG
AATTATTTTATATAAAGTCATTTAAATATCTATTTAAAAGATAGGAAGCTGCTTATATATTTAATAATAAAAGAAGTGCACAAGCTG
CCATGTGA
HUMAN PROTEIN SEQUENCE: hP21-014.2 (Seq ID No: 44)
MPPSPLLKGCAEGWPVRLGPGKVKVCLGPLGARAALRIPGQRCGPRPGRPLRSSQRASERARAADAPGRDPAARMTAPGAAGRCPPT
TWLGSLLLLVCLLASRSITEEVSEYCSHMIGSGHLQSLQRLIDSQMETSCQITFEFVDQEQLKDPVCYLKKAFLLVQDIMEDTMRFR
DNTPNAIAIVQLQELSLRLKSCFTKDYEEHDKACVRTFYETPLQLLEKVKNVFNETKNLLDKDWNIFSKNCNNSFAECSSQDVVTKP
DCNCLYPKAIPSSDPASVSPHQPLAPSMAPVAGLTWEDSEGTEGSSLLPGEQPLHTVDPGSAKQRPPRSTCQSFEPPETPVVKDSTI
GGSPQPRPSVGAFNPGMEDILDSAMGTNWVPEEASGEASEIPVPQGTELSPSRPGGGSMQTEPARPSNFLSASSPLPASAKGQQPAD
VTGHERQSEGSSSPQLQESVFHLLVPSVILVLLAVGGLLFYRWRRRSHQEPQRADSPLEQPEGSPLTQDDRQVELPV*
HUMAN mRNA SEQUENCE: hR21-014.3 (Seq ID No: 45)
TTGGGACGATCATAGAGCGCTAGCACTGAATCAGCCTGGAGAGCGCGGAAGGAAAGGGTCGGTCCGCAGAGGGCGCGGGGAAGGCAG
GGTGGGGACGCGGTGGAGCCCGCGCTCGTTTGCTGAAGGCTTGGAAGTGCAGCGCAGAAGACAGAGGGTGACTAGGAAGACGCGCGA
GCGGGGCTGGCCGGCCGGCGGGTGGGGGAGGGGAGGCGGGGGAAGGCGGCTGAGTGGGCCTCTGGAGTGTGTGTGTCTGTGTCAGTG
TGTGTGTGTGTGTGTGTATGTGTGTGTCTGGCGCCTGGCCAGGGTGATTTCCCATAAACCACATGCCCCCCAGTCCTCTCTTAAAAG
GCTGTGCCGAGGGCTGGCCAGTGAGGCTCGGCCCGGGGAAAGTGAAAGTTTGCCTGGGTCCTCTCGGCGCCAGAGCCGCTCTCCGCA
TCCCAGGACACCGGTGCGGCCCTCGGCCGGGGCGCCCACTCCGCAGCAGCCAGCGAGCGAGCGAGCGAGCGAGGGCGGCCGACGCGC
CCGGCCGGGACCCAGCTGCCCGTATGACCGCGCCGGGCGCCGCCGGGCGCTGCCCTCCCACGACATGGCTGGGCTCCCTGCTGTTGT
TGGTCTGTCTCCTGGCGAGCAGGAGTATCACCGAGGAGGTGTCGGAGTACTGTAGCCACATGATTGGGAGTGGACACCTGCAGTCTC
TGCAGCGGCTGATTGACAGTCAGATGGAGACCTCGTGCCAAATTACATTTGAGTTTGTAGACCAGGAACAGTTGAAAGATCCAGTGT
GCTACCTTAAGAAGGCATTTCTCCTGGTACAAGACATAATGGAGGACACCATGCGCTTCAGAGATAACACCCCCAATGCCATCGCCA
TTGTGCAGCTGCAGGAACTCTCTTTGAGGCTGAAGAGCTGCTTCACCAAGGATTATGAAGAGCATGACAAGGCCTGCGTCCGAACTT
TCTATGAGACACCTCTCCAGTTGCTGGAGAAGGTCAAGAATGTCTTTAATGAAACAAAGAATCTCCTTGACAAGGACTGGAATATTT
TCAGCAAGAACTGCAACAACAGCTTTGCTGAATGCTCCAGCCAAGGCCATGAGAGGCAGTCCGAGGGATCCTCCAGCCCGCAGCTCC
AGGAGTCTGTCTTCCACCTGCTGGTGCCCAGTGTCATCCTGGTCTTGCTGGCCGTCGGAGGCCTCTTGTTCTACAGGTGGAGGCGGC
GGAGCCATCAAGAGCCTCAGAGAGCGGATTCTCCCTTGGAGCAACCAGAGCGCAGCCCCCTGACTCAGGATGACAGACAGGTGGAAC
TGCCAGTGTAGAGGGAATTCTAAGACCCCTCACCATCCTGGACACACTCGTTTGTCAATGTCCCTCTGAAAATGTGACGCCCAGCCC
CGGACACAGTACTCCAGATGTTGTCTGACCAGCTCAGAGAGAGTACAGTGGGACTGTTACCTTCCTTGATATGGACAGTATTCTTCT
ATTTGTGCAGATTAAGATTGCATTAGTTTTTTTCTTAACAACTGCATCATACTGTTGTCATATGTTGAGCCTGTGGTCTATTAAAAC
CCCTAGTTCCATTTCCCATAAACTTCTGTCAAGCCAGACCATCTCTACCCTGTACTTGGACAACTTAACTTTTTTAACCAAAGTGCA
GTTTATGTTCACCTTTGTTAAAGCCACCTTGTTGGTTTCTGCCCATCACCTGAACCTACTGAAGTTGTGTGAAATCCTAATTCTGTC
ATCTCCGTAGCCCTCCCAGTTGTGCCTCCTGCACATTGATGAGTGCCTGCTGTTGTCTTTGCCCATGTTGTTGATGTAGCTGTGACC
CTATTGTTCCTCACCCCTGCCCCCCGCCAACCCCAGCTGGCCCACCTCTTCCCCCTCCCACCCAAGCCCACAGCCAGCCCATCAGGA
AGCCTTCCTGGCTTCTCCACAACCTTCTGACTGCTCTTTTCAGTCATGCCCCTCCTGCTCTTTTCTATTTGGCTAATAGTATATCAA
TTTGCACTTATCTGGACGCACAGAACAGTCTCTCCGTGGGAGGAGACATTATGGGGCGTCCACCACCACCCCTCCCTGGCCATCCTC
CTGGAATGTGGTCTGCCCTCCACCAGAGCTCCTGCCTGCCAGGACTGGACCAGAGCAGCCAGGCTGGGGCCCCTCTGTCTCAACCCG
CAGACCCTTGACTGAATGAGAGAGGCCAGAGGATGCTCCCCATGCTGCCACTATTTATTCTGAGCCCTGGAGGCTCCCATGTGCTTG
AGGAAGGCTGGTGAGCCCGGCTCAGGACCCTCTTCCCTCAGGGGCTGCACCCTCCTCTCACTCCCTTCCATGCCGGAACCCAGGCCA
GGGACCCACCGGCCTGTGGTTTGTGGGAAAGCAGGGTGGACGCTGAGGAGTGAAAGAACCCTGCACCCAGAGGGCCTGCCTGGTGCC
AAGGTATCCCAGCCTGGACAGGCATGGACCTGTCTCCAGAGAGAGGAGCCTGAAGTTCGTGGGGCGGGACAGCGTCGGCCTGATTTC
CCGTAAAGGTGTGCAGCCTGAGAGACGGGAAGAGGAGGCCTCTGGACCTGCTGGTCTGCACTGACAGCCTGAAGGGTCTACACCCTC
GGCTCACCTAAGTGCCCTCTGCTGGTTGCCAGGCGCAGAGGGGAGGCCAGCCCTGCCCTCAGGACCTGCCTGACCTGCCAGTGATGC
CAAGAGGGGGATCAAGCACTGGCCTCTGCCCCTCCTCCTTCCAGCACCTGCCAGAGCTTCTCCAGGAGGCCAAGCAGAGGCTCCCCT
CATGAAGGAAGCCATTGCACTGTGAACACTGTACCTGCCTGCTGAACAGCCTGCCCCCGTCCATCCATGAGCCAGCATCCGTCCGTC
CTCCACTCTCCAGCCTCTCCCCAGCCTCCTGCACTGAGCTGGCCTCACCAGTCGACTGAGGGAGCCCCTCAGCCCTGACCTTCTCCT
GACCTGGCCTTTGACTCCCCGGAGTGGAGTGGGGTGGGAGAACCTCCTGGGCCGCCAGCCAGAGCCGGTCTTTAGGCTGTGTTGTTC
GCCCAGGTTTCTGCATCTTGCACTTTGACATTCCCAAGAGGGAAGGGACTAGTGGGAGAGAGCAAGGGAGGGGAGGGCACAGACAGA
GAGGCTACAGGGCGAGCTCTGACTGAAGATGGGCCTTTGAAATATAGGTATGCACCTGAGGTTGGGGGAGGGTCTGCACTCCCAAAC
CCCAGCGCAGTGTCCTTTCCCTGCTGCCGACAGGAACCTGGGGCTGAGCAGGTTATCCCTGTCAGGAGCCCTGGACTGGGCTGCATC
TCAGCCCCACCTGCATGGTATCCAGCTCCCATCCACTTCTCACCCTTCTTTCCTCCTGACCTTGGTCAGCAGTGATGACCTCCAACT
CTCACCCACCCCCTCTACCATCACCTCTAACCAGGCAAGCCAGGGTGGGAGAGCAATCAGGAGAGCCAGGCCTCAGCTTCCAATGCC
TGGAGGGCCTCCACTTTGTGGCCAGCCTGTGGTGGTGGCTCTGAGGCCTAGGCAACGAGCGACAGGGCTGCCAGTTGCCCCTGGGTT
CCTTTGTGCTGCTGTGTGCCTCCTCTCCTGCCGCCCTTTGTCCTCCGCTAAGAGACCCTGCCCTACCTGGCCGCTGGGCCCCGTGAC
TTTCCCTTCCTGCCCAGGAAAGTGAGGGTCGGCTGGCCCCACCTTCCCTGTCCTGATGCCGACAGCTTAGGGAAGGGCAGTGAACTT
GCATATGGGGCTTAGCCTTCTAGTCACAGCCTCTATATTTGATGCTAGAAAACACATATTTTTAAATGGAAGAAAAATAAAAAGGCA
TTCCCCCTTCATCCCCCTACCTTAAACATATAATATTTTAAAGGTCAAAAAAGCAATCCAACCCACTGCAGAAGCTCTTTTTGAGCA
CTTGGTGGCATCAGAGCAGGAGGAGCCCCAGAGCCACCTCTGGTGTCCCCCCAGGCTACCTGCTCAGGAACCCCTTCTGTTCTCTGA
GAAGTCAAGAGAGGACATTGGCTCACGCACTGTGAGATTTTGTTTTTATACTTGGAAGTGGTGAATTATTTTATATAAAGTCATTTA
AATATCTATTTAAAAGATAGGAAGCTGCTTATATATTTAATAATAAAAGAAGTGCACAAGCTGCCATGTGA
HUMAN PROTEIN SEQUENCE: hP27-014.3 (Seq ID No: 46)
MPPSPLLKGCAEGWPVRLGPGKVKVCLGPLGARAALRIPGQRCGPRPGRPLRSSQRASERARAADAPGRDPAARNTAPGAAGRCPPT
TWLGSLLLLVCLLASRSITEEVSEYCSHMIGSGHLQSLQRLIDSQMETSCQITFEFVDQEQLRDPVCYLKKAFLLVQDIMEDTMRFR
DNTPNAIAIVQLQELSLRLKSCFTKDYEEHDKACVRTFYETPLQLLEKVKNVFNETKNLLDKDWNIFSKNCNNSFAECSSQGHERQS
EGSSSPQLQESVFHLLVPSVILVLLAVGGLLFYRWRRRSHQEPQRADSPLEQPEGSPLTQDDRQVELPV*
HUMAN mRNA SEQUENCE: hR27-014.4 (Seq ID No: 47)
TTGGGACGATCATAGAGCGCTAGCACTGAATCAGCCTGGAGAGCGCGGAAGGAAAGGGTCGGTCCGCAGAGGGCGCGGGGAAGGCAG
GGTGGGACGCGGTGGAGCCCGCGCTCGTTTGCTGAAGGCTTGGAAGTGCAGCGCAGAAGACAGAGGGTGACTAGGAAGACGCGACGA
GCGGGGCTGGCCGGCCGGCGGGTGGGGGAGGGGAGGCGGGGGAAGGCGGCTGAGTGGGCCTCTGGAGTGTGTGTGTCTGTGTCAGTG
TGTGTGTGTGTGTGTGTATGTGTGTGTCTGGCGCCTGGCCAGGGTGATTTCCCATAAACCACATGCCCCCCAGTCCTCTCTTAAAAG
GCTGTGCCGAGGGCTGGCCAGTGAGGCTCGGCCCGGGGAAAGTGAAAGTTTGCCTGGGTCCTCTCGGCGCCAGAGCCGCTCTCCGCA
TCCCAGGACAGCGGTGCGGCCCTCGGCCGGGGCGCCCACTCCGCAGCAGCCAGCGAGCGAGCGAGCGAGCGAGGGCGGCCGACGCGC
CCGGCCGGGACCCAGCTGCCCGTATGACCGCGCCGGGCGCCGCCGGGCGCTGCCCTCCCACGACATGGCTGGGCTCCCTGCTGTTGT
TGGTCTGTCTCCTGGCGAGCAGGAGTATCACCGAGGAGGTGTCGGAGTACTGTAGCCACATGATTGGGAGTGGACACCTGCAGTCTC
TGCAGCGGCTGATTGACAGTCAGATGGAGACCTCGTGCCAAATTACATTTGAGTTTGTAGACCAGGAACAGTTGAAAGATCCAGTGT
GCTACCTTAAGAAGGCATTTCTCCTGGTACAAGACATAATGGAGGACACCATGCGCTTCAGAGATAACACCCCCAATGCCATCGCCA
TTGTGCAGCTGCAGGAACTCTCTTTGAGGCTGAAGAGCTGCTTCACCAAGGATTATGAAGAGCATGACAAGGCCTGCGTCCGAACTT
TCTATGAGACACCTCTCCAGTTGCTGGAGAAGGTCAAGAATGTCTTTAATGAAACAAAGAATCTCCTTGACAAGGACTGGAATATTT
TCAGCAAGAACTGCAACAACAGCTTTGCTGAATGCTCCAGCCAAGATGTGGTGACCAAGCCTGATTGCAACTGCCTGTACCCCAAAG
CCATCCCTAGCAGTGACCCGGCCTCTGTCTCCCCTCATCAGCCCCTCGCCCCCTCCATGGCCCCTGTGGCTGGCTTGACCTGGGAGG
ACTCTGAGGGAACTGAGGGCAGCTCCCTCTTGCCTGGTGAGCAGCCCCTGCACACAGTGGATCCAGGCAGTGCCAAGCAGCGGCCAC
CCAGGAGCACCTGCCAGAGCTTTGAGCCGCCAGAGACCCCAGTTGTCAAGGACAGCACCATCGGTGGCTCACCACAGCCTCGCCCCT
CTGTCGGGGCCTTCAACCCCGGGATGGAGGATATTCTTGACTCTGCAATGGGCACTAATTGGGTCCCAGAAGAAGCCTCTGGAGAGG
CCAGTGAGATTCCCGTACCCCAAGGGACAGAGCTTTCCCCCTCCAGGCCAGGAGGGGGCAGCATGCAGACAGAGCCCGCCAGACCCA
GCAACTTCCTCTCAGCATCTTCTCCACTCCCTGCATCAGCAAAGGGCCAACAGCCGGCAGATGTAACTGGTACCGCCTTGCCCAGGG
TGGGCCCCGTGAGGCCCACTGGCCAGGACTGGAATCACACCCCCCAGAAGACAGACCATCCATCTGCCCTGCTCAGAGACCCCCCGG
AGCCAGGCTCTCCCAGGATCTCATCACTGCGCCCCCAGGGCCTCAGCAACCCCTCCACCCTCTCTGCTCAGCCACAGCTTTCCAGAA
GCCACTCCTCGGGCAGCGTGCTGCCCCTTGGGGAGCTGGAGGGCAGGAGGAGCACCAGGGATCGGAGGAGCCCCGCAGAGCCAGAAG
GAGGACCAGCAAGTGAAGGGGCAGCCAGGCCCCTGCCCCGTTTTAACTCCGTTCCTTTGACTGACACAGGCCATGAGAGGCAGTCCG
AGGGATCCTCCAGCCCGCAGCTCCAGGAGTCTGTCTTCCACCTGCTGGTGCCCAGTGTCATCCTGGTCTTGCTGGCCGTCGGAGGCC
TCTTGTTCTACAGGTGGAGGCGGCGGAGCCATCAAGAGCCTCAGAGAGCGGATTCTCCCTTGGAGCAACCAGAGGGCAGCCCCCTGA
CTCAGGATGACAGACAGGTGGAACTGCCAGTGTAGAGGGAATTCTAAGACCCCTCACCATCCTGGACACACTCGTTTGTCAATGTCC
CTCTGAAAATGTGACGCCCAGCCCCGGACACAGTACTCCAGATGTTGTCTGACCAGCTCAGAGAGAGTACAGTGGGACTGTTACCTT
CCTTGATATGGACAGTATTCTTCTATTTGTGCAGATTAAGATTGCATTAGTTTTTTTCTTAACAACTGCATCATACTGTTGTCATAT
GTTGAGCCTGTGGTCTATTAAAACCCCTAGTTCCATTTCCCATAAACTTCTGTCAAGCCAGACCATCTCTACCCTGTACTTGGACAA
CTTAACTTTTTTAACCAAAGTGCAGTTTATGTTCACCTTTGTTAAAGCCACCTTGTTGGTTTCTGCCCATCACCTGAACCTACTGAA
GTTGTGTGAAATCCTAATTCTGTCATCTCCGTAGCCCTCCCAGTTGTGCCTCCTGCACATTGATGAGTGCCTGCTGTTGTCTTTGCC
CATGTTGTTGATGTAGCTGTGACCCTATTGTTCCTCACCCCTGCCCCCCGCCAACCCCAGCTGGCCCACCTCTTCCCCCTCCCACCC
AAGCCCACAGCCAGCCCATCAGGAAGCCTTCCTGGCTTCTCCACAACCTTCTGACTGCTCTTTTCAGTCATGCCCCTCCTGCTCTTT
TGTATTTGGCTAATAGTATATCAATTTGCACTTATCTGGACGCACAGAACAGTCTCTCCGTGGGAGGAGACATTATGGGGCGTCCAC
CACCACCCCTCCCTGGCCATCCTCCTGGAATGTGGTCTGCCCTCCACCAGAGCTCCTGCCTGCCAGGACTGGACCAGAGCAGCCAGG
CTGGGGCCCCTCTGTCTCAACCCGCAGACCCTTGACTGAATGAGAGAGGCCAGAGGATGCTCCCCATGCTGCCACTATTTATTGTGA
GCCCTGGAGGCTCCCATGTGCTTGAGGAAGGCTGGTGAGCCCGGCTCAGGACCCTCTTCCCTCAGGGGCTGCACCCTCCTCTCACTC
CCTTCCATGCCGGAACCCAGGCCAGGGACCCACCGGCCTGTGGTTTGTGGGAAAGCAGGGTGGACGCTGAGGAGTGAAAGAACCCTG
CACCCAGAGGGCCTGCCTGGTGCCAAGGTATCCCAGCCTGGACAGGCATGGACCTGTCTCCAGAGAGAGGAGCCTGAAGTTCGTGGG
GCGGGACAGCGTCGGCCTGATTTCCCGTAAAGGTGTGCAGCCTGAGAGACGGGAAGAGGAGGCCTCTGGACCTGCTGGTCTGCACTG
ACAGCCTGAAGGGTCTACACCCTCGGCTCACCTAAGTGCCCTGTGCTGGTTGCCAGGCGCAGAGGGGAGGCCAGCCCTGCCCTCAGG
ACCTGCCTGACCTGCCAGTGATGCCAAGAGGGGGATCAAGCACTGGCCTCTGCCCCTCCTCCTTCCAGCACCTGCCAGAGCTTCTCC
AGGAGGCCAAGCAGAGGCTCCCCTCATGAAGGAAGCCATTGCACTGTGAACACTGTACCTGCCTGCTGAACAGCCTGCCCCCGTCCA
TCCATGAGCCAGCATCCGTCCGTCCTCCACTCTCCAGCCTCTCCCCAGCCTCCTGCACTGAGCTGGCCTCACCAGTCGACTGAGGGA
GCCCCTCAGCCCTGACCTTCTCCTGACCTGGCCTTTGACTCCCCGGAGTGGAGTGGGGTGGGAGAACCTCCTGGGCCGCCAGCCAGA
GCCGGTCTTTAGGCTGTGTTGTTCGCCCAGGTTTCTGCATCTTGCACTTTGACATTCCCAAGAGGGAAGGGACTAGTGGGAGAGAGC
AAGGGAGGGGAGGGCACAGACAGAGAGGCTACAGGGCGAGCTCTGACTGAAGATGGGCCTTTGAAATATAGGTATGCACCTGAGGTT
GGGGGAGGGTCTGCACTCCCAAACCCCACCGCAGTGTCCTTTCCCTGCTGCCGACAGGAACCTGGGGCTGAGCAGGTTATCCCTGTC
AGGAGCCCTGGACTGGGCTGCATCTCAGCCCCACCTGCATGGTATCCAGCTCCCATCCACTTCTCACCCTTCTTTCCTCCTGACCTT
GGTCAGCAGTGATGACCTCCAACTCTCACCCACCCCCTCTACCATCACCTCTAACCAGGCAAGCCAGGGTGGGAGAGCAATCAGGAG
AGCCAGGCCTCAGCTTCCAATGCCTGGAGGGCCTCCACTTTGTGGCCAGCCTGTGGTGGTGGCTCTGAGGCCTAGGCAACGAGCGAC
AGGGCTGCCAGTTGCCCCTGGGTTCCTTTGTGCTGCTGTGTGCCTCCTCTCCTGCCGCCCTTTGTCCTCCGCTAAGAGACCCTGCCC
TACCTGGCCGCTGGGCCCCGTGACTTTCCCTTCCTGCCCAGGAAAGTGAGGGTCGGCTGGCCCCACCTTCCCTGTCCTGATGCCGAC
AGCTTAGGGAAGGGCAGTGAACTTGCATATGGGGCTTAGCCTTCTAGTCACAGCCTCTATATTTGATGCTAGAAAACACATATTTTT
AAATGGAAGAAAAATAAAAAGCCATTCCCCCTTCATCCCCCTACCTTAAACATATAATATTTTAAAGGTCAAAAAAGCAATCCAACC
CACTGCAGAAGCTCTTTTTGAGCACTTGGTGGCATCAGAGCAGGAGGAGCCCCAGAGCCACCTCTGGTGTCCCCCCAGGCTACCTGC
TCAGGAACCCCTTCTGTTCTCTGAGAAGTCAAGAGAGGACATTGCCTCACGCACTGTGAGATTTTGTTTTTATACTTGGAAGTGGTG
AATTATTTTATATAAAGTCATTTAAATATCTATTTAAAAGATAGGAAGCTGCTTATATATTTAATAATAAAAGAAGTGCACAAGCTG
CCATGTGA
HUMAN PROTEIN SEQUENCE: hP27-014.4 (Seq ID No: 48)
MPPSPLLKGCAEGWPVRLGPGKVKVCLGPLGARAALRIPGQRCGPRPGRPLRSSQRASERARAADAPGRDPAARMTAPGAAGRCPPT
TWLGSLLLLVCLLASRSITEEVSEYCSHMIGSGHLQSLQRLIDSQMETSCQITFEFVDQEQLKDPVCYLKKAFLLVQDIMEDTMRFR
NNTPNAIAIVQLQELSLRLKSCFTKDYEEHDKACVRTFYETPLQLLEKVKNVFNETKNLLDKDWNIFSKNCNNSFAECSSQDVVTKP
DCNCLYPKAIPSSDPASVSPHQPLAPSMAPVAGLTWEDSEGTEGSSLLPGEQPLHTVDPGSAKQRPPRSTCQSFEPPETPVVKDSTI
GGSPQPRPSVGAFNPGMEDILDSANGTNWVPEEASGEASEIPVPQGTELSPSRPGGGSMQTEPARPSNFLSASSPLPASAKGQQPAD
VTGTALPRVGPVRPTGQDNNHTPQKTDHPSALLRDPPEPGSPRISSLRPQGLSNPSTLSAQPQLSRSHSSGSVLPLGELEGRRSTRD
RRSPAEPEGGPASEGAARPLPRFNSVPLTDTGNERQSEGSSSPQLQESVFHLLVPSVILVLLAVGGLLEYRWRRRSHQEPQRADSPL
EQPEGSPLTQDDRQVELPV*

TABLE 5

MOUSE GENOMIC SEQUENCE: mD28-002 (Seq ID No: 49)
CGCGGCATGAGCCCCGCGTGCCCCACTGCTCGCCGGGCTGCTCCGAGCCGGGGTGCTCCGGGGCTCCAAACTCGGGCTGGCGGGGCA
AGTGTCTTCATGAACCCAGAGGATGTCCGGGAAGCACTACAAGGGTCCTGAAGTCAGTTGTTGCATCAAATACTTCATTTTTGGCTT
CAATGTCATATTTTGGGTAAGTTGGAGCGCTCCTGGGTTTCCAAGTGCTGTGGCGGATGGGTGGGCGACGGTCCCCCGCCGTGGTCG
CTGCTTTACTTTTCGCCGCCCCCGGAGCTCCTGCCCCAGTCGCGGCGAGAGGCGTCTACCTTCCGCCTTCCCGGCTCGAGCGCGGGG
GAGAGAAACACTTCATCCTTTCCAGGACTCCGAGGAGGTGAAAAGAGGAACCAAGCCGGGCAGGGGGCAGCCAGGCCTCGGGGTGGC
TTTTTTCTTTCTTTTCTTTCTTTCTTTCTTTTTTTTTTAAGGGGGTGGGGGGCGCGAATAGCTGGGGTACAAATGGGAGGACGGCTC
GGCAGTTTGAGAAACGAGGGAGCACACGGATTAGATTTGCTTCCCGTGGGATCTGGATGTTGGGCAGAGCGTGGTGTCATAATGGGG
CAGGCTAATCGGGCAGAGCCGACCCCGAGATCCACCTTCCAGCGCCGATCTGATTGGCCGAGGGCTTGGCTCGGTGTTGCCGCGGCT
GGTCTGTGCCGTGCTCAGCGTGTATCTCCTTGCACCATCTCCGGCTTTGTGTAAGATGCAGATGCCGTGGTGTGTGGCTCTGTAATG
TGCACGTAAATAACACGTCCTGCATGCTCCTCTCCGGCAGTAAAGCGCTCCCTTTGCATAATGGACTGGCCAAGTGGGCGTGCAGGG
TGGAGGGATTTATTTCTCTGTGTATCCTTGTGGAATAACTAGTCATCAGTCAACATATGTTTCGGTGTGCGCGTGCCTCGCGTCTTC
CCCTTTTAAAGGGGTGACACACAGGTTTTCGGAACCCTTGGAGACATGCAAAGGAGAAGGTGTGTAAGACCTGTTTTGCTGGCAGAG
CAGGTGAGCCGCGGCTTGCAAACCGGCTCCCAACCGTGCTCTGCGTGACACTCCCTCGTGCCGGGTGCCTATTGTAATCAGGCTGGG
ACCCTACTCTTTTGTTGTGATTCTTTTCTGAGACCTTCCAAACGTGTTCAGCAACCAGACAAAGAGTTCTCTAGGACAAGAGGGGAA
ATGGTTTGTTGGTTGGTTGGTTGGTTGGCATTGGTTGGCAGACCGTGTTATTTTTCAGCCCACCTATTTCCATCCGGGGTCGCCTTC
CTCCCTGGCACCTCTCTCTCCATGGGAGGGAAAGATGGTGGCGGACCATAGGGATATCCCCAAGTTTCTGCGAGGTAAACAATGATC
CAGTGGTCTGAATCCAGGACATTTTAATTTGGACTCCCCTTAATTGTGGCTGTTTGCTGTAGACATAAACATCTGTGAGAATAGTTG
TAGTTATGCTGATCTTCTGCCCACCCCCTCACTTACCAGCTTATTTTATTTTCTCTCAGAGTAGCTATGTGTGTTTTGTGAAAGGAA
AGGAAATGGACTTTGTGTCAGCATGTCATCTGTCCTCAGGACTGGACCAGATTGATGAGGGAGTGTGGGAACAAATACCATATACAG
CCATTCACGAGTAGTTTTGTTTTTTTCTGTCAGTGATAATATATGGTTTCTGGGAGCAACTCCCCACCCCTGCCCCCCTGCTGCAAA
CAGAGCAAGAGGAAATGCGTTTCTTTCTTTCCTTCTTTTTTTTTTTTTTTTTTTTGCTTCTCTCCTGGACCCTTTCTGGAAATGTTT
AAAAGTGGTGGCTAGGCATGCTTCCCATTGAAGTATTCTGATTGCCTTGAACTATGAGAGGCAAGAGAGGCAAGGGCCGTCACATGG
GGCTGCAGGGGTGGTTCGCCCGGGCCTCTTCTTTCTGCTCCAACACTTCCAAAAGTTGCACATTTCAGCTACCAGGCAACAAAAAGC
ACCCACGGGCTTTCTCTGCAGTTTCAGAGCGGGGAGTGAGAGGGAAATAAAAAGGAATGCGGAAATGTTTCCCTGGCAGTTTGGTGT
AATTTTGTAGCGAGCACAAAGGCAAATTTAGCCCTGTGTCTGGATTAGGCTGCAGCAAACCCCCTGGACTGTGGAGAGCTTCAACAA
AATTGCAGATTGCCTGCATGGAGAGTTTCAGAATAGGACCCACATCTTGTCTGATTTTTGAAGTATTACTGTGGGCGGGCAGTAGGT
AGGCTTGTGGCAAGTCAGAGAACGGTCTGAGGCCCTGAAACTGAAGGAGCTGCCCTGGGCAGCATTGTTAAGTAGGTGTCTACTTCA
GAGAGTCATTTGCTCTCCCTGTCCTTCCAGTGCAGAGGGCCACCTCTAGTTCGTTTTCTATACTGCTCCTGTCTCCAAATGACCTCA
TATTTAAATGTCCTATTTTATCAGGGCCAGTGGGCTCTAGCAGAAATTAGCAGGAAAACATCCACGTTATCATTAAAAGGAATACAC
ACCAAAATATTAGTGACCGGAGATCTGGCAGCAGCTGCCAAAAACATGCACACTTCTGTCTTCGGCCACCAACCTGCCCGTGACTGT
CCAAGATAGCAGAGTCAACACTTCAGTTCAGTTGTACGGTTCTGACGGAGGAATTGGTAACAGAGACCATGGGCTCCCGGTCTTTCA
TCTTCACATTGACGTGCAGAACTAAATTCGAAATTCATTGAAGACATTTGAACGTAATAGTTCTTGTAAAATAAGAGGGAGCGCACA
GTGGCAGCTGTGACAGCTCGGATCTCTTGCTAATCAGCACTCTGTCTGTGTTAGTCAGTTATCTGCCCTGGGCCTTCGTTACTCAGC
TATTCGAATGAAGAATTTGGTTGAATTGTGAGGTTTTTCTCTGTCTCTAACAAAGACTTTGTTGAGTTCTAGTACGCCAGCCAGGCA
AGATCGAGTCTCAGGGATGAGCAGTAGGCTCTAATGATGGACTGGAGACTTGCCTATAGTTATAGATGGTCCTTTAGTAGACATGCT
CACGGACTAATTTGTTTTTCATTCTCTCCTCAATGATCTTTTTTCACTTAAGAAGAAACAAAACAACAAACCTTTCAAACTGAATTT
TCCTTGCTTGTTGAGTTATTGGTATGGCACGCATATTTCCCTATCTCTCCCCTGAATCTCATGTGTGACGCTCTGAGTCATTAGCTG
TATTTTTATTATTAGAATCACAGCTTCATGTGGAGGATCCTAATTCTTTTATATAACAGCAGTGCTGATATTGTGAGTAAGAAAGTC
AACATCTTGTACAGATTCCTTTCTTGTGAGTTAGCACAGGGCGGTGGGGGGGAAAGCCTTGTTTTTCTTGCTCTATATTGGAAGAAC
TCAAGCCAGCAGGAATTGATTTTATTGAGGGTGTCACCAGAGGAAGACATGTATCAGAAACACTGAGTTGTCTTTGGGGCGGAGGAG
AGAAGAGAAGAAGGTTTGTTTTAAGGTCAGTCCTTTGGGGAGTTAATTTCTCTGTCCTGTCACTGACCGTTTATAGATGTTTTGTTC
TGGTTCTTGACACTTAACTGTTTCTCAGAAAGTGAACGTCAACATCCTGGCTACTTATTGCTGCACAGCAAGCTCCCAAACTTTAGA
ATTTCAAAACGAAACCTCATCATGATAGTTCATAATTCTGTGATTTGACTCCTCGGGGGGCTACTTCTAATGTGGGACCTCCGTGCA
GTTGCTGGGAGTTTCTTGCTGTCTCCTTTGGGTGGGGGAACCCCGGGCTGGTGGTCACCTGTGATTGGTGATGCTTACCCATGGCTC
AGTGTCGCTGTCAGCTGTGGCACAGCAGGTGGATTTTTCTGAGGTTCGAAGCAGCTTCCAAGCAGTTTAACTGTACTGTCACTTGGC
ACATTTAGTCTAAGTCCTCATGGAGTCTGCTCAATTGAAGGTTCCATCTTTGGATAGGGAAGTGTCAGGGTCACTTTGTAGAAGGTT
ATGTGGTGGGCTGGCTAGTTTTATATCCACTTGACACAAGCTACAGTTATCTTAAAGGAGAGCATCTCAACTGAAGAAATGCCCCCA
TAAGGGCCGGCTGGAGGGCATTTTCTTAATTAGTGATTGATATGGGAGGGACCAGCCCCACTGTGGGTGGTGCTGTTCTTGGGCTAG
TGGTCCTGGGTGCTATACGAATCAGGTTGAGCAAGCCATGATGAGCAAGCCATGATGAGCAAGTCATAGTTTCTGTATCAGCTCCCG
CCTCCAGGTTCCTGCCCTGCTTGAGTTCCTGTCTTCACTGCTTTGATGAACGGTTTATGTAACTGCAAGATAAGTAAATTCTTCTCT
TCTACCCCTGGTTGCTTTTGGTCATGGTGTTTTATCACAGCAATAGTAACCCTAAGGCATGCGGATTTCAGCCGTCATTGGAAAATG
ATTTCTTAGAAACCATTGATGCGCTGGTCAGCAGAAGCCTCGAGTTGGATGGTGGGACCACTTGATATTTTGTTCTCTGTGCTGTCC
CAGGACTAGAACAGCTGTTGGCTGAAGCCAGATGTCTCTTTGGTGTGTTTTCAGATATTGCTTTGTGTTGCAGGTTGGAGGTTTATC
CAGAGTAAATGGCGGAATGTTGTATATGCTTTGTTCCCACAGGCCAAGAGGAGGGAGATTCAAGGAATCTTGGGTGGTGGGGTGGGG
CGCCAAGTAGGATGTCTTATCTTATGATTCAGTATAAATTTCTAGATGTTCTGGAATGATGGTACATGAGCCGTTTATAAGTCATAA
TTTTTGTTTTAATTTGCTAGACATCTGTTAGCTTCGATGGCAACCATATGGGAGTCTAAATTGCCTTTTGAATTTCAGGGAGAGGGT
TGGAAGAAAGCTAAATGTGGCTATGGAAGTGTCAGGTAGGGAATGTGTGCAGACCTCAGAGGGGAGAGTCACGATGCACCCAGTGTC
TTGCCTCTTCCAGCAGTTCACTGTGGGGCTGATGCGTTCTCAATGGACGATGCTCTTGAAGGTTGAAATGGAGCCACTAGCAAAAGT
CGGAAAACCAGATAACATTCCGCCCTGCATAGAAGTTATGAAAAGGGTGAAATAAACACTTCTGCAGTGGGACTTGGAGTAATCTGA
GGGACATTTTATATGCTTTTAAAAATCAGAATTTAATTGGGATGCCAGATTTCTAGCAACTTGCATCTTTAGTTTCCCAAATAATCT
GGAAGTTAGCATTTGATAATGATTTTTTTAGAAAAAAAAAAAAAAAAAAAGAAGTTGCATTTGGGAGCTCATTTAGTTGGATCTCTC
TGTCCCAACATTCAGATTGGCATGCCTTTCCATACAACAGGGGAAAGCAATGTGTTCTTTTGATTTCTACATGGGGCCTTGCTCTTT
AACCCAGTGTGGTCTCTAATTTTGGCTCCTGTGCTACTCCTCCATCTCTCACTAGCGGCTGAGGCTTTTTAGCTATAATCTGCTGTG
CCTAGAACCGTGTGTGTTTTCAAAAACCCCACAGCTTTACCATTGTGATGTCCTTTTTAAAAGCCTGTAAAAGTATCTGACAAAGAC
CTATGGTGATATTTTTGTGACCAGTAAAAGAAATTGGAGCACAGAGATGTGAAATGGCTCTCGTGGTGTTGGAGCGCTTGCAGGTTC
GCTTGGGGAGTGTTAAGTCGACGCTGTAGCTAGAGCCCCAGTTTCTCAGACTGAAACTTATTTTAGAAGATGCTTGTTATTCTAAAA
TTAAACATCAAGTAAGTCATTTGCATCTGTTCATAATTGAAAAGTGATAAAGTTTTTGCATATGCCACAATTAACACCACCCACAAG
CACCCACAAAAAAAGTTTAGGATGAAATGTTCTTAAAGAGAATGCTAGGATATAATAAATTATTATGTAGTCTGTTGAAAAATTCAT
AAATGACAATTAGAGTATTAATGAGGGTATTTTTTTTTTGAGTCACATGTTTATTTCTACGCGTAATACCAACTGCCTTAAGGTGAC
TAACCTCAGGATTCTAACTTACTGTTCAGTACGGGATGATAAGGGCCTCCTGTTTGTGAACAACCATCTCCCAATCGTTTCCTTAAG
CCTCGTGTGTCATGTGTTGTCTGGAACACATAGGCATCTAATAACTGCTTGTTGAATGGATGAGCTTCCGAATGTTGAGAAAGCCCA
TGGGGTTTATCCTTCAAGCCAAGGAAGCCTTGTCTTTCACAACAGGCAGTTAGCATCTAAGGGGTGTTTGTCTTTGGAACATTTAGT
TAGAGAGCTGCGTCTGTCTTCAAACCAGATGTAAGGTCTCTCTGAAAATTTCAGCCTCCTAGTTTACTGTTGATTTTTCTTTTTTAA
TTAAAAATATTTTAAAAATATTATGTATATGGGTGCTTTGCCTGCACATATGTCTGTGCACCACATGCATTCCTGGTTCTTGCAGAA
GCCAGAGGAGGATGTTGGATCCCCTGGAACTAGAGTTACAGATGGTTGTGGGCCACTGTGTGGTTGCCAGTGCCCTGAAACACAGAG
CCACCTCTCCAGCCCTCCTGGTGCTGACTTCTCACTTGAATTTCTTTTCTTTTCTTTCTTTTTTTATTTTTGGTTTTTCGAGATGGA
GTTTCTCTGTGTAGCCTTGGCTGTCCAGGAACTCACTCTGTAGACCAGACTGGCCTCCAACTCAGAAATCCACCTGCCTCTGCCTCC
CAAGTGCTGGGATTAAAGGCATTCACCACCACTGCCCAGCCTCACTTGAATTTTAACATATATCTCAAACCTACAGGAAATGTTAGA
GGAAACAAATGATAAAGTTTGCTACTTCAGACATGTTATTCATATATCTTTAAGTTTTCCAACTTGTTTTTATCATGGTTAAGGACA
AAGTTGTCTGCCTGATGGCTCTCACAAACACAGTGTGGATTTCGTCTCGAAGGGTTCTCTCCTTGATAGCCACAGTGCCACCATCAA
AGCTAGCAAATTACTCCCATCCACTTCCCAGGCCTTGGGTCTCCTTCACTCTCCCAATCTCAATAATTCCATTTAAAGCAAAGGAAT
TCAGGCCAGAACGGCACAATATGTGGGGTTCTGTTAAGGCTCGTCCAGTCCCTCACTATCAAGAGCGTGTGACTGGCATTGCTGGCT
GCCCTCCCTGTGGGTTGCCTGTGGGCTCCTCAGGCGCAGGTTCTGGCTGTGTGTTCTGGGGTGGCAGCGGCCAGAAGGGACGCTTTG
CTTTCATCCGAATCCCATACGTGGCAGTGGTTGTTGCTTTAGGCGTTTTCTCTGCCAAGTGTCTCCGATCATGTTACTAACTTTTGT
TGCTTTGGCAACTTGTAACTGCTTTTTGGCAAGCCACTTCGAAGCTATAGAACTGCCCATCTCTCCTGGACTTCCTATCAGTAATGA
ATGTCTGTCAGCAGCGTTTCATGGCTTCTCCTTTTCCATCTTGGTATGTTTCAACAACGAATCCAAATTAACCCCTTTTCTCTTAAA
GTTGCTTCTGTAAGGCATTGGGTCACAGTGATAAGAAAAGTAAGCAAACCCTTTGCTCTCTTTAAAAAGTTAATTTATTAATTACAT
CCTAATTGCTGCCCCCCAGAGCTCCCCTTCACAGGGTCCCTTCTCCCCTCCTCCTCTGAGAGGGTGGGGCCCCCCCTGGATATCCCC
CCTTCCCGACCCTGGTACATCAAGTCTCTGCGGTGTTAGGCACATCCTCTCCCACTGAGGGCAAACAAGGCAACCCTGTTAGGGAAT
GGTCTCCACAGCCAGGCAACAGCTTTAGGGATAGCCCCTGCTCTAGTTATTGGGGGATCCTCATGAAGACTGAGCTGCACATCTGCT
ACATATGTACTGGGGGCCCTGGTCTAGCCCATGTATGCTCTTTGGTTGTGGCTCAGTCTCTGAGAGCTCCTAAGGATCCAGGTTAGT
TGACTCTGTTGGTCTTCCTGTGGAGTTCCTATCCCCTTCAGGGCCTTCAATCCTTCCCCCAACTCTTCCACAAGAGTCACCAGTGGA
CAACTTTGTTTAGCTGTGGATATCTGTATCTGTTTCAGTCAGAGGACAGTTATGCTAGGTTCCTGTCTGCAAACATAACAGAGTATC
ATTAATAGTGTAGGAATTGGTGTTTACCAATGGAATGAGCCTCAAGTCGGGTCAGTTAATGGTTGGCCATTCCTTCAAGTCTCTGCT
CCGTCTTTGTCCCTGAATTTCTTTTAGACAGGACAAATTTTGGGCCGACATTTGTGGGTTGGGTTGGTGTCTTTACCCCTCCTCTGG
TGGTCCTGTCTGACTACAGGAGGTGGCCTCTTCAGGTTCCATATCCCCGCTGTTATGTGTCTCTTGGCTAAGGTCATCCAGATTGAC
TCCTGGGAGCCTCCCCCATCCCAGATCTGTGGGACTTCCTAGAAATTTTCCCCAGCCCCCAACCCCAGCAGCTGCAGATTTACATTT
ATTTACATTTATTCTCCTGGCCCTCAGGCCCTCTCTCCTGTCTCTCCCAACACCTGATCCTGCCCCTCAACCCTTTTCTCTTACCTG
CCAGTGCACTGGCATCCTGGACCACAGCCCTTCATCCTTATGGGACATCCCTCTTTCTATGGTATGAGTTCTAGGCTCTTGCAGCAT
CCTTCTGCTGCCCTGCTTGGTCCTCTCCAGACACTCATATTCCTCCCCCATGGTCCTTCCTTATTTAAAAATAGCCTTGATGCCATT
TCTCAAACCATTTCTCATCTCTGGAGGATCCCACACCCTCCTTGTGTCTCATGGATAGGCTGTGCTCTGGCCTGATCATAGCCTCTG
GTCATAGTGTTCTGCCCAAGGTCTTATTTGCATGCACTTTCCTTAATGTAGGCACCAGAATGGGACACGTTTTCCATAAGTTCTTCA
GTCCCTCTAGAGGGCTGTTACCTTTGAGCTCTCCCCTTCTACACAGAGCTGCATCTTGAGCCAGGCTGTGCTGGTCTGGCCTGGAAG
AAATTACTATCACATTAGAGAGTGATGTTTAGTTACTCATCAGAAAGGCTGGGCAACACATTTCCAGAGTCGTCTTCCTGTATTAAA
TGAGATTATACATGTGAAAGCACTTGGTAAGAGGCAAATCTCCCTGTGTAAACATTAGTTATTAACCCAGGCTAAGCTGTACTCATA
TCCCAACAGCCAGGGAATGTCGCTGGCTCAGGTTGAATTTCTGGCGAGTTTAACCTTCCGTCTTCTTTCAAATGAACTTTGGCTAAG
TCTATCCATCCAGGGTGGACACTAATCTCTGAAGCCTTAACTTTTACCCTACAGGTTTCAGTTTGGACCTGTCATTGCATAGATCTC
CTGCTTACGTGGGTATTTCTGGCCCATTGCTTTTCAAACTTAGCTGCACCCANNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNGGGGAGAGAGGGGAGGAAGGAGGGGGGAGAAGAAGTAGAAGAAGAAGAAGAGGAG
GAGGAGGAGGAGGGAGAAGAGGAGGAGGAGAAAGCATTATAACCTTTAGAAGTCCATGGGACTCATCTTTCCAGCCATGGAAGCCTA
AATGTCTAAGGTATGTTTTGAAAGCTTGCTGCTGGTGCTGAGGCTGTAGCGCACTGATGCCATTCTTGCTCAGTATGCACAGAGTCC
TGGCTTGTATCCCCTTCAGTGACAAAAATCAAAATGATCGGCAGGTGGTAGATGGTTAGATGACAGGGAGTTAGCAAGTACGTACAT
AAATTAAGCAGCAGTGCTCAGATGGGCTTTCCTCTTGTAGTCTTCACCATGGGTGAGGAGACTTCTGGAGGATATGATGATTGGCAA
TTTTACCTCCCTTTGGACAGCAGCAAGCTAGAAAGCCTCAAACTTTTCATTGAGGTGTGTGTGTCTGGGGGGCATCTGGTTCAGTAG
GTCCAGACAGGGTATAATTTGGTGGAATGAGTGTTATATTAAAAAAAAAAAAGTCTTACAGCTGACTCGAAAACCCAGGCTGTGGTT
TATTTTTTCAGGGTGGGAGAGATGTGTCTTGACCCTTCCTTCCGTTTTTGAGGGAGGCAGATGGGCGGTGTGCAAATACTTGTGAAG
GGTGATTGATTGCACCAGCACCGGAAGCTGCTGCTATTCACTTACGGGGATAAAAAAAATGAAAAATGTAGCCCAGTTAATCCCGCA
AGGAATTTCTTATTAATCCAAAAGTGCCAGGTTATTTTTAAACCCGTGGGCCTTAAAAACAAAATGAGAGCATGTCTTTAAAGAGAG
CGTGTGCCATAGTGGGTATGCCAACAGGCAGGGCCGGAGAGGGAAGGAAGCCAGTTAGAGCAGGCAGGGTGGGAAGCTCTGCACCCG
CCTGTGGGATCTGCCTGTGTCACCGCAGATGGAGAGCTGATTACCACACACGGCCTCCCTCTGGAGCACCCACAGTGCTTCCTGACA
ACTGTCCTATCCTGGGCTGCCCAGACCTCTGACCCTGGGCACTCTCTTCAGAGGGAGGCCTTGACAGAGGTGACTGCTGCAGAGCAG
GGAAGGAACATTCCTTCTCCCAGATCCAGGTCATCAATGTAGCTGCCAGGACAGGTAACAGCAAAGCAAACGTGGGAGACAAATAGG
ATGTCTCAGTTTTCTTTGCAAAGAAGTTTCAGTGTATAATATCCAAAGCTTTCTGCATAGAGCAGGCTCCCTCCCTCTTTCCTTTCC
TTTCCTTTCCTTTCCTTTCCTTTCCTTTCCTTTCCTTTCCTTTCCTTTCCTTTCCTTTCCTTTCCTTTCCTTTCCTCTTTCTTTGCC
ATTCTTCTACCTCCTAGTCTCTCTCTTCCTTCCCCCAGCCCCTTAAAAATTACATATTTTGATTATAGCCAGGTACCCAAAGTGGAG
ATATTTTGAAAAAGTTGAAATTTTGAATAAAAATATCCATTATCGGAGACATTTCACTGGGGCTAGACCATCTATCTCCTAGAATGG
AAAACAAAACGAAAAGAAATAAAAAGCTTAAAACCAGAGTGGCATACAAAAGCAATGTTATCCCTGTGGTACTATCCGTATGTGTGT
AGAGGATTGCTAATTATGAATTGTAAGGTGTAATTTAAAAATGTATAATCACCAACCTTATTAGCAGAGTAGTTTCAGGAGTGCGTG
GTAGGACAGTTTGCCACCTCGCTGCAGGCTTGGCGATGCTCTGCCCGCTTCCAGCAAGTGTTTACTGTCTACATGTAGATTGCATGA
CATAGGCTCATCTACTTGCCCAACGTTCTTTCTAAATTACTCTTGGCACCAAGGCTTGAATTTCTAAAAATCTGGAGGGAACAGCTG
GTGCTCTAATAAATATTCAGGCCAGCTATTTTAATGCTTTGTACTCAATTAAGTAGTCCTAATAAAAGAGGAGCCCTTGCCCTAAGC
ATTTCTCAAAGAATAATGTTTTGCTTCTAACAAAAAACTTTCTCCCAGTGTTAGTTAATATTTCTCCCAGGCAACTTTAAAACTTCG
TTGCATCCAGTAGGTCTTTTGCCCATTCGTGACGAGGCTCAGTCCTGAAGTCTGCACTTCCTTGGCCCATATCTTTTAACTGACGAC
GACCATAGGCCACTCATTCACTTTCTAGGGAAATTCTCGGGCTTTGTTTTTTGAGATGGTCTGTTCAAGACTGTCCTTCTGACTGGC
CTTGAACTCATGACTCTTCAGCCTTAGCATCGCAAGTGCTGAGATCGCTGACATGTGCTACCACATCCAGGGTTTACCTGACATAGC
TTTACACAGTAACCCATCGTGCAGCTTGGCTTAGGCCTCTGGTGCTCTTGGGTGATATGGAGTCTGCTGTTAAGCATTTGGGCTTCA
AGAGTGCGGCAAGTTTTCTTCTACACCATTCTAGCCTTGGGACCTTAGGCAAATTATTTAGTTGCTGGTGAAGTCTTCTTTTGCAAA
ATGGTACGTTTAGTAGTAGTTTTAATATTATTATTTTGTTAGGACCTGCATTTGAATGCAGTATTTTACAAGTACCAAACAAGCAAA
CAAGCAAGCAATAGTCTTACCATCAAGATACATCCCCAGCAATTTATATTCTCCTTCACCTCTATTTTGAGACACAGTTTTACTGTG
TATCTCTGTGTATTTTGAAACTTACTATGTAGACCATACTGGGCTCGAACAGTCAGAAATCTGCCTGCCTCTGCCTCCTGTGTGCTG
GGATTAAAGACGTGGAGCACCACTGGTTAATTACTATTCTGTTTTTCTTTTTAAAGATTTATTTTATGTATATGAGTCCACTGTAGG
TGTGTTCAGACACACCAGAAGAGGGCATCGGATCCCATTAGAGATGGTTGTGAGCCACCATGTGGTTGCTGGGAATTGAACTCAGGA
CCTCTGGAAGAGCTGTCAGTGTTCTTAACTACTGAGCCATCTCTCCAGCCCTAATTAATATTCTTAACGGGACTATTAAAACCTAGA
ATAATCTATCTCAGTTGTGAGGATGAGAGGAAAGATGTGTAAGCTGCCTGAATGTCCCATCTGTATTGGCAGTTACTACTGGTTTCT
CTACCCCATCATGTCTTTCGTTAGCTTTCCATCCCTTCTTCAGGACCGTTAAGATAATGGTACCATGTAACATACACTTTCTGCTCT
CTGCGCAAGTGTATTCTCAGAATAACTGAAGAAAAGTAGATCCAGCAACTGTTCTCAGTGCTCTTCAGACTGTCCTGGATAGAACAG
AATTCCTATCATGAAGACCAGGCCATGAATTCATAGCTATTGTTTATTTATTGTTATTCTCTTACTTCCAGTACCCAGTGATTTGTT
CAGTGCCGTGACCAAATACCGGTGAGAGACAGTGAAAGGGAGAGAGAGATTACTTTTGCTCACCTTTCAGAGGTATAGCCTGTCATG
CTGGGGAAGGGATGCCGTGTTTGTGGCTGGGTGGTTGTATGGTTAGAGCTCCTCACATTTTGATAGGCCAAGATGTGGAGAAAGTGG
AATGCCGGCTCTTGTGTTTTCTAGGTGGTCAAGTCAACTTGACAATGAGACGAACCATCACAGCTGGTTAGAGTGAATCTTTTTTTT
TTTTTTTTTTTTTTTTTTTGCAAAACCATACTGCCCCATATTAGAATTTTGTACCAGGAAGTCAGGTTTCAGAAGCTCAGCTCTGCT
ACTAAGCCTGACATTTAGTATCAGGGTGTAGGATGGGGATTGAAAGTACTTTATTAGCGTCTCCTAAATGTGGGGATTCTAAAGCTC
TCTAGGTGGCCTGACTATAGTTCTAGCTTTTTATTGTAATGTAAACACTACATATCAGTGCCATGTTTTATCACACACACACATACA
CATACACACACACACACACACACACACACACACACACACACACACTGCATTCTTCACGTCATGAGAATGAACACCCTGCCAGAGATA
GCAATGACTTGTGAGTTAACCCTCATGTGCCCTTTCAAGTGACGCCCAGTTAGGCTTTGGCCTGTACCACCCACATCACCGAAGGAT
TGGGCCACTCATCTCTGTGAATGAGTGATGGACTGACTGGCTTCTCTGGTACTTAAATGAGATGTGCTGTGCACTTGTATCTCAATA
TGGCTGGCTGACATGGAAGGCACTGGCTGCCATAGCTCACCAGCTCACCACGGTCTCCCTTAACCCTGGCAGAAAAGGCATCACAGT
TCATGCAAGGCTGGAGGCATAAAGAACCTTGCTCCTGGGTTGCCATGTAGAAGGTGTGCCGAAGACCCTGTACTTTGTCCTCTGGAA
CCCTATCATCTCTAGGAATCTTACTCCCTATGATTCCAAGTGCAACCTCCAAGGGCCAATCATTGTAAGAGTAGCATGGCCTTGGAC
AAACTTGTGTCTTCACAGAGTCTTTCAGGTGCCTGCATGGTGTATGAGACTCCCGTAACCCATACTTGGGTTTGGTAGTCAAGAAAG
GGGCCTGTGATGTGGCATGTACCTTGAGCAATCTCCCCCTTGATGCTTCCTGTTTGATAATGACATTTTGAGTCACAAGGATCCGCC
CATTTCAAGTTTTTTTTTCTTTTAAGTCAATTCTGTGCTTAAACATTAAAATATACTATTTAAGAAGGGGAAATTAAAAAAAATATC
TCTACTCAGAGGCACACAATGACAAAAACATGGCCTATTCACTTGTAGGTAGCTGTTGGGAACTGCTGGCAGGCCGAGGCCCAGGCT
TGCTTCCTTTGTTATAGAAGTTAACAGGTGTAAAAAGAGTATTTGGCAGTTTGGCACCAGTTGAAGAATATCTCCTTTACCAGAGCA
AAGAATGAATAAAACGGAGAGGAAAGAGCATCCCCACTCAAGGTAATTTATGGCCATGGTTTGAAGTTGTCTCCTCTGGAACCCCAC
ATCTCTCAGAATCTTCCTTTGGGAAATTCCATGTTTAAGCAGTTTGCAAGGCCATATGTGTTTATGAGTTGAAAGTCACAGTTTCTG
GAGGTGCCCTGTTTGTAGAATGTGTTAACATGTTTGTCTTTCGCGTATTCTGTTTCTTTAGAGTGCTGGGCTTGGGAAGCAGAAGTC
TCCTCGACTTCTGAAAACTTGTGAACCCTTGGTCACTGAGTAGTTAGAGTTGAGGTACAAAGGGAAAATAATTTGGGGGTGTATCTC
AGACTCACAGGAAGTCAACCTTAAATGTTTCTGCAGTCTGTCTTAGGAGGCTCCACCTTCTCCTGTTTACTTTATGGTTGAGTTCAA
CATGGCTTAAATCTTGTTATTTGATGCACTTTTAAGGAACAGGATAATTATTGTGTGCTTTTCTATTGGGAAAGCAATATAAAAACA
TGGATAGAATTTTGAAGTAAGGATGCTCATAATCCTAGTAATACTTTCATTGGGTTCAACTGGAACTGGAAGCTTCTATCCAAATGC
TGAAAGCAGCAAACAACTGCACAAAACCAACCAACTACCCAGCCATCCAGCCAACCCACCATCCATCCACCCATCCAGACAACTATG
AACCATCCATCTATCCATCCATCCATCCATCCATCCATCCATCCATCCATCCAGACAACTATGAACCATCCATCTATCCATCCATCC
ATCCATCCATCCACCCATCCATCCACCCATCCATCCAGACAACTATGAACCATCCATCCATCCATCCATCCAGACAACTATGAACCA
TCCATCCATCCAACCCACCATCTATCCATCCATCCAGACAATTATGATCCACCATCCATCCATCCATCCATCCATCCATCCATCCAT
CTATCCATCCAGCGAGCCAGCTAGCCAGCCAGTCATCTTAGAGTTTCTCTCTAGTCATCCGTGAGGCTATGACTTTAGCCTCACTTT
GAAGTGGATACTGCTACATCACTACCCTTTAAGCGTTTGTTTTGCCTTTACTAGCGTCTTCCCCCTCAGGACAACATTGAGAATAAG
TGTTTTCAGCATACAGAGCAATGCCTTGTGAATAAACACTGGTTTCAGTCAACCAGAAAGAGGGAAAGAAAGGAAAATCAATAAACC
TCCTGCAGGCATTTGAGAAACTGTGTACTTTTAAGAGAAGGAATGGCCAGTGATAAGGAGCTTTGCAAAACAATAGCTCTCATCTCT
TCCTTTTTTGAGCCGCTGACTATGACTTTCTGTGCTGAACCTTGTAGCTGGGATTTCCTAGTTCTCTGTTGCCTTGGTTGTGCCCCA
CTTAGCTGGGGAATGACAGCCACTGTCCCTGTGACACCTCAAGGGGAGCAGCTCTACAGCCTTCCTAGATTTTGAATGTCATAGCAG
GAATCCTAAGGAGACCAGTGCTCCTTTGGTTCTGGCTCCTTTTGCTTTGCCTGCTGGTCTTGAGAATCAAGAGTGAAGAGCTGCCTG
GGTGTATATGGCATCTTCATTTTTCCTTAGGTCTGCAAAATTTAACCATTCTTGGTATTCTCGCTGCATGCTTAGAGTGTTCTTCAA
TTGCTATCTGGTGAGAGTTTGAAACAGCTGTATAAGAGTTTGAAATAGTGTTTTACTTCTATTTTTCCTTAAGATGAAGAATCAACA
TTGTAGAAATGAGTGGAAGGCACCAAAGCGACCTGGAAATATCTCTTACACTTTTGTAAAATATATGATTATTTAGTTTAAATACAG
ACATGTGTCACTGATGGCTGTTAAATAGCAATTTCAATATTTGGATTGTCTTAAATATAGATATGTGCTGTCTTTTCGTAGATATGT
ACAGTAATGATCTGAATGACTGATATGTGACCTTTAAATATTTTTATCCATGTTAAGAGAATAAAATGTCTTTATCATAGCATACTA
TAACATTTCAAACTATATGGGACGTTTTGTTTATTTTTTTGAGACAAGTTAGCTCAGGCTGGTCTCTTAACTCATGGCAGTCTTTGT
GTCACAGCCTTCCCAGTGACGGAATTATAGGCATGAGCTACCAGACTTGATGAAATGAAATCTTTTGAAGTTCAACATAAGGCTAGA
GATCAGCTTCGATATTTACAGTGCACACAGGAAGAACCCTGTTGTGTTTCTCATTTGTAGATGGGTCAGAAAGTATATGTATGATAT
GTATAATCATCTCACCCTGAACTGTGGGAGATTGTTGCGTCACAGCTGGGGATAACAGCCCATTCTTTATTGCACCTTTCAAAACAC
AGTTTTAATTTTGAAGTTCCTTCCCATGGAATTTTCCTTCTCTTGCCCCGTTCTTTCTTTGACTTTAACTCCAGGATCGAGGAGGCA
GGGAATCTTCTTAAGGGAGGCTAAACCCCTCTTAGTCTTCAAAATAAAAAGGCTAGCCTGCTGCAGACAGAATAGAGGATTGGGATT
CTTTTGAACAGAAATGGCTGTGCAAATATTTAATCCGTTGTTCTTTAGTTGGTCTATTTAGGGCAGAGTACTGGAAGGAACCGTGCT
TTAATTGGCCTTCTGCAGACCTGATTATGTAACCTTAAGACTTGCATGTTGAAAAGGACGTGAAAAATCCTCTAGTCTCTGTTACCT
ACCAGTCATCTTTCTGCAATCTCCTCTTAAGCATGGATTTTATTCTCCGGCTGTAAGTTATAAAACAGGGGACAGAGCTCTTTTTTT
CTGGCAGCTTTTCATTTTATAGGTCTGAGATGCCCTCAGCCTTACTTGTAGGGGCAGTGTGCAGAGAGGACAGTCAGAGTCCCTGTG
GGTTCGCTCACGGGTCAGGTTTTAGCCTGGCCAGTGGTGAGGATGTCTGTATACCATTTACACACTGATCTGGGGTGAAGGTGGCAT
TTGGCAGTGCTATTGGTAGGATGCCTGAGCGCCGAATCACCCGAGCTGCCAAGTCCCCGTTCTCTCACTCCATTTGGCTCGACTTGA
CAGTTTTGGCTCAGTCATTTTGATTAACATCAACCTTGCTTATGTTAAGTAACTATTCAGGTTATTTATTATGTATTACTCTCATGC
TTTGGAATGAGCGGCCAAGAAACTTCTGCCTCATATGGACTCCATATGCAGCTGTTAAGTTAGCAGAAACACAATTAGTTGGCGCCT
AAGCTGCATATTGAGTAGATTAGGCCGGATCGCACAGGCGTTGTTTGAGATTGTTGTCTCCAATATTTAAGAAAGCTATTAGGTGTT
TGAGGATGTGGGGTGATAAAGCATCTCTGAGTTCACATATGTCCTGTTTTGTATTTTTATTATGGAGTGCAAATATCTGTGGAATCT
TGGTTGTTTGTTTTGCACTGCTGAGCTCCAGAGAGGATTAAAAACATAGTCTGTGAAAAACAGACATCTCTTTATTTCCATTGTTCC
CTCCCCATCCCAGGCTAGGCCGAGAATGTTGCAGTGGGTAAAAGACAGCCTGGGCTCAGATGTAGGCCCTGACACTCCTACCTGTGT
GAGTATGTATAGGCAACATCTTTAGGGTCACCCCAGACACCAGCATGCTAAGGAGTGCAGGTGAACAGGGACTTCCAGAGAGTACCC
CACTGTCACGGTGTGCTCTGGGTCTCTGTCCCTTGAGAAGGGTGTTGTCAGGTTTAGGATGTGTTCCCTGAAGGATGGAATCTGTCA
CTTTTCAAGCTTGCTTTCCATTGTCCTGCTCCCACGGAAGGATCTTGGAAGTCCATCCTTTTCCTTGGCTAGTAGATGGTAAGGTTT
GGCCTGGGGACAGCTGCCACATGCTCTTACCGTGTCCATGTGTTGCGACGGTCTGCTTCTGTGCTGTCACCGAGCTTTCCTCTTACG
TGGGGACCCTGTGTGTGCGCATTGTGTGTGTAGGTGCCCCGGCGTGCATGGGTGTGACGTGGACGTCATATCTCAGACGCCGTCCAC
CTTGTTTTCTGAGACTGGGTCTCTCCCTGGCATGGAACTCACCAGTTAGGTTGCACTGGCATACAAGACATCTCCTGTCTCTGCCTT
GTGAGTACTGGGATTGCAAATGTGTACCACTGTACTTAGCTTTTTTTTTCTTTGTTTTTTTAAAGTGGCTTTTAGGGAATCAAACTT
AGGTTCCCTATGTTTGTGTAGCAAGCGTTTTACCAACTAAATTATTTTCCCAGCTAGGGGGTCCTTTTGAGGCCATTTATATCCGCA
TTATAATTACAATTAAATATACATAAGTATGCCTAAAAACTGCTTAAAATATACTGAGTAATAATTATCCATTGAAATAATTTAATT
AAATTTGAAGTAATTTTTTTTTTCATTAAGGAACAAAATAAGCATCCATTTGGCCCCCACTACAAAAAAATTGCTATACACAAATAG
GTATGCTTATGAAATTACTTAGTTTATTAGCATATAATTCATCCAGATTATTACATTCTCTTAATTGAATTTACACTTGTATGCTTA
TAATTTTAAAAATAGTTTTAAACAAGAGGTGTCTACGAGCTGCCGAACATTAAGGGCTCTGGTATTTTTAGTTGTCTGAGAGGTAAA
ATTCTAGGTCTAAAGGGAAAACATGGATGCTATAGTAAAAGATAACTCTCCTGCTCTGTGCTGTGAGCATCTGCAGCAGAGTTTGGA
TTGATTTTCACCTTCTGAGCTGTCATAGACGATTTCTGTTCCTTCCTTGACATAGGAGATTCCTGTAGCTCAACCCTTACCTAGTCT
GAGGCCATCCTCTGCCTCTTCACAAAAGTTCTAAAGGTGAAAAAGTGGGCTGGGGAGGGAGATGAGAAGCCAAAGCTGAGGTTGGTT
ACCACACCGAGGGACGTGCTTGCTTCTTGGAGTTGTGTCTTGTGGATGTCAAAGCCCCTTTCCAGGAAGGGCCTTACTGTTTGCTGC
CGTGATGGGAAGAGTGTATTGAGGCTCGTGAGAAGCAATGGCTGAGATTGGGAGCTGCAGAGGGATACTCATCTATGTTTGGAAGCC
CACACAGGCATTGGGTGATTTCTTGGACAGCGTTCCTGGCGGTTGTAGTGACAGTGAGCTTTACCTGGGCCATTGCTCTGGTTGTTC
ATAGGGAGCAGCCTTCATCCTTCAGACATGATCTAATGTCTTTCTCCAGACAAAGAGCAGCCTCTGTGGACAGTGGGGCCCCAGAAT
CCCAAACATTGCTCCTCAGCTATGATGCCTACCCACTGCATGGACAGCATTCACCTTTGACCTTTAGAGCAAAGAGAACCCAGGCCA
GAATGATGTTCCTGCTGCCTCCTGTGCTCTGTATGGTAAGTCCTTAGTGCCTGGTCCAGAAATCTTTGTCGTCCTCTTCTTCTTCTC
CTTCTCCTCCTCCTCCTCCTCCTCCTTCTCCTCCTTCTTCTTCTTCTTTTTTTTTTCCTGAGACAGGGTTTCTCTGTGTAGCCCGGG
CTGTCCTGGAACTTACTTTGTAGACCAGGCTGGCCTCGAACTCAGAAATCCGCCTGCCTCTGCCTCCCGAATGCTGGGATTAAAGGC
GTGTGCCACCAACGCCCAGCTTCTTTCAGGGTTCAAAAAGCAGACGCAAAGGCGAGGGTTGGTAGTGCCTCATAGCTGGTCAGGTTT
CTTAGCGGCAAGGATGTCATCTTAGATACTTCTCTGTGTGTCAGTGTGCCTCCAGGCAAGCAAAGGAGACAGACCACAGTACCTTCA
GGAATTGCGTCCTGTGTTGGATGAGATTGAAGCTGAGTCCAAGTGTGATTGATTCTTGGTCGTAGAAAAGGAGTCTGACACCAGGAG
TGTGGGCAGCCCCTTCAGGGACAAGCTGTAGCCCATCTGAGGCAAAAATGCAGGGTTAGCCAGATGCCTGGCCCTTAAGAACAGATG
GGAGTGTCCTAGCAAGGTGCTCCTACCACGAGGCAGCAACGGCCTGTCTTGACAGCAGTGGGCCTCTGATGAAACAGGGCCAGAGAG
ATCTCGAAATCTGTGTCTGGTATTTGTAAGTTGTTTTCAGGCACTCAGCCATGCTGTTGGAGAAGGCCTCAGAGCTCACCTCAGAGC
TCACCTGAGGCACGTTAGGGACGAGTTGATGATTGATTCTCAGTCGCTCAGCTGGGCGGAGCCAAGGCTAGTCCTTGGTTCAGAGCA
ATTTTGAGTGCATTTCTTTGTGAGTTCTACATGCACTTGCCATCTGTACTGGGACTCTTCACCCCCCCCCCCCACTCGCCGAGAGGG
ATAATATATAATACAGGTTTAAAATGAAGACTTTAGATGCTAGATGGTAAGAGGTGCCCAGGTAGGAAACCTGATGTGGAATCTTAG
TCCTATGATTTCCAGGATGAATCTTACAGGCATCTAGTCAGACCATAAACAGAAATGACATTTCGTTCACTTAATGATAGGTGAAAA
ATTATTCACTGGCTGTGGCTGCTAATTGAGGTCCAGCATGAGTCAGGCTAGAACTTAGGAAGTCAGGGACAGGGTAAAAAATAAGCA
TGGATCAGGAAGTGGATTTTAATAAGATATCCATGGGAGGGGGACTCTTGCTATGTGAGGGTTAGATAAATATGATGATTTCCACTG
GTGCTGTGCTGTGTGCTTTGGAAGTGGGAGGGGTGGGGAAGGGGAGTCAGGGCTCCTAAAGTGCCACTCTCCAAGCTTTAGCTGACA
TAGAAAGCAGGCGGTGCTTGTAAAGTCAAATGCTTTTTGGCATTTCTGGTGGTGTAAATTCCATCGTGTCTCTACCATTTTGAAGAA
CATCTTGGTGTGGTAGGAGTGAGGGGCTTCTTGTGGTAGGGCTCCCTGCTAAAGGAGAGAGGTGGCTCAGCAAGATGGCTGAGGCAT
GGGGACTTTGCAGAAGGGCCCAAGACAACACCCACTCTGAGTACTTCTCAGCCAGTCCCCACAGGGAACAACACCAAACACTGTGGT
TGCTGAAGCTGTGCCCTTGAAGCTAAGGGAGCATGGAATGCTAGTGTGAATCAGAGTGGGTGTGGCCATTCAGGGACAAAATTCCCA
TCCGTTGGCTGTGGTTCTTTTTATCTTTAGTTAATGATTTCATTCTTTAAAATTAAAAAACAAAACAAAACAAAACAAAACTCCACA
CGTCAATTTTCACATGTTTATGGTGTTGTTTGTGGATTAGTGGTCTGTAATTACCTCAACTCCTGGGCACAGTATATTTGTTATCAC
AGTGAAGAGGGCGTACCAATTATTTTAAAAAGCAATTTCTCTTTTAATTTATGCACGTGCGATATTAGCTAGCTTAAATGCATATGC
ATACAAAGATACGTAGTCTCTCTACATTGACCTGGCTGTCCTAGAACTCAGTACGTCGATGAGGCTGGCCTCAAACTCACAGAGCTC
CACCTGCCTCTGCCTCCCAAGTTCTGGGAACATAGGTATGTGCAACCATGCCTGGAGAAAGATAATTATATATAGCTATTTATTATA
TAGCTATTTTATTGCAAATAATTAAAGGAAAGAAAGACTTGAATATTAACATACTTTTCATTGCCATTGAACATAAATTCTAAAATG
CTGAAGTTTTTTTCCAGGGGTTTAGAGAGGACTGAAAAGACACTTAAAGTAGGGCTGTTCCCAACTTCACATCCAGGTACCATAGTT
GCCTACTGTCAGCGCCTATTTCTGGGTTACCATTTGACGTGCGACACTGCCCATGCTAACTTCTCCTAGTCCCTTGGCTCCTGTCAA
AGGAGCTACGTCAGTGCATCAGATGCCTCCCTAGGAGCCTAGAGGACCTGTCCTGTTTGTCACCTGCTTTCCCTCTGCCTCTCTACA
GACACACAGGGCTTGTATTTGACAGATTTGATAGTGGGAGTCAGAAGCTTTTGGTATCAGAGGGTTGGAGTTTGAATCTGTCTGTCT
GTCTGTCTCTGATGGTGGTGCTGGGATTCCCAGCCATCATATCTCTTGTTTATTTAATAGGAGAGTTATTTTGCCTCCTGTTAAATG
TTCGAGTCTTGGTTTCCTTGAGGATCAAGGGTGTGTTGGGAGACAGTTCTTTTAGCTCACTTGTGTTTTTGCATGGGGGTTGGTTAG
GGTGTTGGTTGCTTTTGATCTGAAGGATGTCTCTATAAAAAGAAGCCTAGTAAGGTTGGTGACAGTCACATAACATTTGGGCTCCTT
AAGCTCAGGAATCCTCAGTTACTCTGTGAGCCCTCGGCAATGTCCTCTGGCAGTCTGGGCTTGGGGAACCAGCACTTGACTTCTGGG
AGCTAAGGCCTGCCTGAACCCTGGATGAAACCCCATCATTTAGGTCTTTTCCTACAAGGACCCAGACAGCCATAGTAAAATGGGCCT
CTGAGTCAGTGCCATTGGGGGGGTCATTATGACAGGGAACTATGGATCCTGTAAGGTGGAAGGAGAGATCTGATGCCTGAACGTTTT
CCTGACTGCTATATGTCACACAACTCATCCCCCCCCTCCTCTCTTGCACACACACACACACACACACACACACACACATTTGTCATA
TATAATACTGAGGAATGTGGGTCAGTGTGTAATGTGTGCTTAGCCGTGGGTCCAGTATGTAACATCACAAAAACAAAACAAAATTCT
AAATTTGTTGTAAAATTTGAAGGGACAGTTAATGTAACTTTTCCTTTTGGTGAGTTTGGCACATGGGGTGTGGATAAGATCTCCCAG
TCAGAAGTGCCACTCGAGGCCCTGTGATTTCTTTAGAGTGGCTGGCTCAGGGCAGCCTTCTTTGTTGTTTTGGAGTCTGCCCTTTCT
TTTCTCTCCCTGAACCCCATGTTCTACCTGATGATCTTTGAGTCACGGCAGATTGTTTTTTAAGGGATGTATCAAAGTTGCCTTTAA
ATGGTTGACTTACAAGCTGGCTGTTAAAGCGAGTTTTACAGGCTACCAGATGAGATGGAGTCAGTGGTCTGGCTAGGTGTACTGTTG
CAACAGTTTTAAAAGACAGTGCCAAGATGAAGACATCCATCAGCAACCCATTGCGTTACATAGGGGTCCCGTGGTAGATGGTACCAT
CACAGTTCCTTGTCATGATGCCCCCCACCCCCCAAAGGTGTCTCACTTAGATCTTTCTGGTCCGTGTGGGAGAAGATCTAGATGAGA
GACTTTCATCCTAGGCTTCGGCAAGGCTTGGCTCCCAGAAGGCATTCCATAAATTGTTTTGTACGCCTGAGTGACTAAAAAAACATG
AAAGTGCTTAGTTAACTGTAAAGTGTTAGTTGCATGTGAATTACTATCCCCCCATCCCTGTCTGCAAAGTAGACCGATGCTCAGGTG
TGGCAGAGGAGAGCCAAAGGAGGAGATCTGGGGTGATGGAAAGTGGCCTCCACACTAGCCTTCCTCTCCTGAAAGGTTGACAGGCTA
GGCTGTGAGCAGTTTCCTCCCTCCCTCCCTCTCCTCCCTTCCCCTCCCCTCTCCTCNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NCCCCAAAGGTGGAACAGTTTCTAAGGAGCAGATGGTCCCAGCTCCTGTTATCCATTTATAACAACCCCAGAGATCTTTCTAAAGGA
TGGGTGTCGTTCATCTCTTTTACCACTTTCATCTTATAGACTTTAATTATTTCTACCAAAATTCTCCCCATCACGTTTCTGTCTTCA
TCTGTAGGGATTTTATCTGGCACCATTTTCTTGTGTCCAAAATATCTTTTCTCCCAGAGCTATAGGCTCCTTTCATGATTCTTGGGT
GCTTTCTGGAACCCTCTGCTGAGTGAGCTGGAATATATCTGGTAGGATAAGGGCAAGGTCACTATGTGCTTTTAGAGTCTGTCCTTG
GGACAGAGAAGCTACCAAGGAACTCCTTCCATGTGTGGGCTGATTTCTGCAGCAACCACTTACAAGCGGCACAGGTGAAACCATTCA
TTTAACGAGTGGAATTTATAACAAAGGAGAAAGGTACATTTTCATGCATGTCACTTGATAGTATTTGAAATAAAAGTTGTCATTAGT
TTCTACTGTCATGATGATTTTGTTTTAATTAACAAATTGTTGAGTGTGTTTGTGTGAGTGCCTACATGTTAGCGGGAGGATCAGAGC
AGCGCTTCCAAAGGCCAGATCATTTCTTCCACTGCGGTTCCAGCGACTGAAGCCAGGCTGTCAGGTTTCTGCAACAAGTATCTTTAC
CTGATGGGTTCTGTCACCAGCTCTGGTTGTTCTGTAAGTGTCTGGGAATCCTTGCTTTTCTATGTAGACCAGAGAGTCTAGATGCTG
TTAGGATGTATTTCTTCATATAGCACCATATAACTTCGGAGGTACTGGTTCAGGGTGGCTCCACCGCTGTAACCTTTGCGGCTCCTT
TATCGGATTTCTCTGCAAGGATGACTGTCAATCATTGTGACTTGTGTTTGAGAAACAAAGTTCTTCCTCAAAATGATGAAAGTAAGC
GTGGACTGGATGTGATTTTAAGATTTTCAATTTGGAAAAGCACCCAGGGTTCTGGTGCTCCGTCTGTCTATAGAGGAAGGCTGATGT
CATCACTCAGCGTCACTCAGCAACTTTGTCCCTGGCAGTGCTACTGGGTGGCATTCTAAACAGGTCACCACATACATGGAGAATTAC
TGAGTAGTTTAGGGGGCCGCGTCTTGTTAGGATCGCTGAACAGCGCGGTGATTTATGGATGTGGAGAGCTGTGGCAGGGTAATTTTT
AGCATTTACATTTCATTTTACCACAATCTCGAGGGAATTTTGAGGCTGCGTGAACAAGATTACAAGGCCACCCTCAGGCAATTTAGG
ATTATATAAGGAATCTTCTTTGCTCCCTCACACCCAGGCTCCGTCTTTCTAGCCCACCCAGAATCCTGGTAACCTTACTCCCTCCCA
GATTGGGCGTGGGTCTGTTAGTCCTTCTCTATTGACACCACCCTGGTCCAGCTTCCAGCCTCTCCCTGAAACTGTTACCTACATCCT
GATTTTCTTCTATGGTCCCTGTAACCAACACCTGTGTCAGTTTGTCCTTGCCCCAAAAGTGACCTGTCTCCCTTCCCCAGAGTTACC
TTGAGGTCTTATGCCTTTCTTCCTTCCTTTCCTTTTCTTCCTTCCTTCCTTCCTTCCTTCCTTCCTTCCTTCCTTTCTTTCTTTCTT
TCTTTCTTTCTTTCTTTCTTTCTTTCTCATTCCAGCAATTCCCCTGCCTCCCAGTTAGGTTAACCCTTAGAGGATTCTGTCATACTT
CGCATGCCATTTCCTTCCAAGTATCTTATCCTAACTGGGTCAGATCACTGTTACATGTTCTGGTGATTAATTGGGAACTAAGCGTTC
AGTTTGAAGCGGACGGAGCCATTAATAGTTATATTCCACGCCCACAGAGGGCCTTAGATAAGTGCTCTGGGAGGTTTGGTTTTCAAT
GATCATGTGGTTTACAGCCGTAATCCCTGCAAAATTCTTAATACTGACCCCTTTGACAGTTAGTAATTTGAGGTGACAGTATGTATG
GTGATCTTTTTTAAAGTCCCAAGGTGTGAAGCCGTTGAAGTAAAGGACGGAGTTCTCAGGCTTCCAGAGATGGAGAACGGGCTTGTA
GGTGATGCGGTTTTATTTCACTCCTGACTGAAGATACATTTTGCTCTAAAATCTGGCTTCGTATGCAGATGCAGAGGCTTTTTACAT
TTTGAGGAGGGAAGAATATTCTCTGTGCACCTGGGATTTTGGAATGTGATGACGAGGGGCCCTGGGCTGTTACAGAAATGGCAGATA
AACAGAGGGTGGTGAGGGAGGCCCTTCTGCCAGTCAGAGGCTGACAGATTTGATCAGAAGGGGTGGAGGCTGCTGCTTGTATTTTGG
GTAGATTTTCTCGTTCCAAAGGGACTTGGTCTCTAGGTGGCCATTAGTGAGGGGAGTTATGTGATCAGTGGGACCATCTGATAGTGT
CTGTGTGTGTGTGTGTGTGTGTGTGATTAGTGGGGGTGGCATGTGATCAGTGGGGGCTTGTTACTAGCTGGGCCACACGATTAGAGA
GACTGATCATGAAGGCCATGTAGTGAGTGGCGGAGGGGGGGTGCGGGGGGGGGAGCTGTTTGGTTCATTGGGCCATTGATGAGGGTG
GGTCATGTGATCAGTAGGAGCTAGGTGGTTAGTGGTGTGCCAAGGGGACTCGGGCTTCCTTTTCTGTGCCCATGTATACTCAGAAGG
TAGGGTAGACAGTGCCCAGTGAGCTTGATGAATCAGAAAGAACTTGAGGCCCTGGTTTCTAAGACCTGCTCTCAGGACATCCCTGGA
GACTCAGTTCCTCCCTTTGACTCCTGCTGACATTGGCCTGCCTCAGGGAAGGCTCTGAAGAAAGGAGGCACAGCATGCTTTGCAAGG
TACTTTCAAATTGCTGGGTCCCTTCAGGGGAAAAGAGCCTTTTGTGACACTGGTCTAAAGATAATTTTTATCAGATTCTGTAGCCTG
AGGTCTCAATAACAGCAAAAAGCCCTTCCAGGGCCTGGCTTCCATGGGGGTTGGACCTGATTGAAGTAGCGGATTCCTTCATGCTGG
CCCTGCAGCAGGCCCAGGTACCCCAGGAGTGTTTCCCTGAGAGAACTGTACAGTCAGAAGGAGGTGCCAGAAAATGAGATGAACGTG
CGAGCCCTGGCAGGTCGACTGTGCTTGCTAAGGGCAGGTGTGTGCAGTTCTCACATCACGTATCTATCTCCACAGGGTCAGGGTGGA
ATATGCTCATGACAGGTCACATGCAAATGAGGTTTAGAAGGACGGGCTCTGTTTCATTCCTTTCTGAGTATCAAAGTCATGGGACAG
GGTGTGCTTCACTTGCTGTGGAGAATAGAGCCACTCTAGCGTTCGAATGTGGGGGATTCACAGGGCTCATGATGATTTGCCAGCTAG
ATGATTCAGCAGAGTCTTTGCAAGGTTTTGCTAATGGTTAAATATCTACAGCAGGCTGAATTTGGATTGGCTGAGGGACAAGGTGAA
ATAAGGTAATCATAGTTACATGATTTTTTTTTTCTTCAAGTTCTGGCATCTTGTGTTCTCTGCCAAGCAGAAATATTCCGGCAGAAT
CGTGGTAATGCCTCTCATCTGCAGGCTTCCAGATGGCAGCTTCTCTGCCAGCGAGCAGAAGAGAGCTTTGGAAGCCCAGGGGTATCT
CAGTCCTTTCCTCGGTGTGACCCAGGGAACTCTTGTTCCCTGTTTGTACAGGGTGCCGGTGTCTCTTCAAATGCCTTGTATGAGGCC
TGGTGTGGTAGTGCACAACTGTTATTCCAGCATTTGGGAAGCTGAGCCAGGAAGATTCTTAGTTCGAGGCCAGCCAAGGCAACATAT
TGAGATATTTTGTCCCCACTGGAAAAAAAAAAAAAAACCAGATAAAAAGCCATCAGATCTTAAATTTCAAAGTTGAAGGGTGCCATT
TTCCCAATAGCAAATGACCACCTGAGTAGAGCCTGTCAAAGGCCTTCTCAAGAGAACTGTCATCTAAGTGAGCCTTGTTTGTTACAG
ACAGAGGCACTCCCTCCTGTGCGCAGCACAGTACACACTGCAATGCTCCATGAACACTGTTTTTCTAGCTGTGACTCACCCTGTGTC
ACGCCCTCCCCAGGCAACCATTCACTTTAGTGATGTTTATAGATGCGTTTGTCTGGGGCTATGAGATAATTGGGTTACATCTTCTAC
TGGGGGAGCCCATGGGGAGGGGAGGAAAGACACACACAGAAGAAAGTGGTGTGTAAGCCTGAGGGTGGGGCAGAAGTGAGCATTTAA
GGTTGTGCCAGTTGAAGGATTTTTAGGGATTTGCTGCACACAGCGTTGGCCATATTTCCCTATGTGTTCCAGCAGCCTCTAATAGCT
ACCGGCCTTCTCTTGAGGGTCTTGGTTTCGGTCTTCAGCGTTCTTCCCCTTCAGTCCCTTTGAGAACCTTTACAGACTACCCTGGGC
GTGCCCTACGGAAGCTCTTACACTGTTCTGTGTAGATACTCTGAAGCAACGGTTATTTTCCTTGCTAGCCTCGACTGTAACTCACTG
AAGGCAGGCAGTCACATCAAATGCAGTTAGTACAAATGGCAGGCTCGGGCAGTATTAGGGTCAGATAGACCGTGGTGACAGACCTTC
AACACCACTCTTAAATATAGCAGTGTGTGTGATTTTGAAAACATGGTGCATTCTGTAGGCCTGGGGAGATGGCTCCGCCAATAAAAT
ACTTGCCTAGCAAGCAAGAGGATGGGAGTTAAGCTCCTGCTGCTCACATGAAAGGCCCAGTATGGTGGCATGTCCCTGCTATCCCAG
TGCTAAGAATTAGAGACCGGGGATCCCTGGAGTGTAGTGGCCAGCCACGCTAGCCAAATCATGAACTCCAGGTTCACTGTGAGAACT
TAGCTCAGAGAATAGGTGGCGAGCAAAAGAGTAAGGCACCCAACCTCAACCCCTGGTCTCTACACACACACACACACACACACACAC
ACACACTCCTTATGTGCATACACACCATTGTTTTTGTTTGAGAAAAGCACAAACTTGTAGGCTGCCCATCATCATTTACTTGATGTC
TGTTCATTCATCCATTCTTCATTCATTCGTTCATTCATTCACAGGCTATACCTTTTTTTTTTTTTTTTTTATAAGACCAGATTTTTC
TCTTCAGTGATATTTTTTGAATCTTGGATATTTTTACTGCTCTGGGGATTTTCTTTGAACTTGATAATTATTTTGATAAATCTTTAT
CACTTAGATGGTAGCTTAGAGTTAATTCTTTCTATTAAGTACTTTTTAGAAGATAAACCTTTGGAGAAATCACCACATTTTATTAAT
GACTTGTGAGACTTAGCAGCGACTTACGTTGATAATAATGGAATGCGCTTTGGGTAGAAAAATTCTTCTTGCTGACACACCAAGGTG
GAGATGAAGATGTGTGCAGATTGTTAATTTAGGCCAGTGTGTCAAGATAACAGAAGGCTGGCAGTAGTCTCTGTTGATTTTTCTCTT
GTAGCACCTAAGAGGCAATGGGCACAAGCTTGTGCATGCCAACTTGGACAGCTCTCCATGAAGTGCCTGCGGTATTCTTCATGTGGC
ATGGAGAGGGCCTGCTGGGGTATGGAAAAGTTATGGGGCTTGCCCATGGCCATGGTTTCGTGCCCACAGATAACCCCTCCAAAACTG
GAGGTTTCTGGTTATACAGTATAGATTTACAGTTGGAAGAGTTTGTAATTGTTAAACAATAAATATTCTTTTGCCAGTTGATGCTCA
ATGCTTCTTTTAAACAATATATATATATATATATTTATGATTATTTAGCATTGCCTTTCTTAGTTGTGCTAAGCTGTACATAGCTGT
GACAGTTAATTTCGAACCCATTTGAAAACAATGTTGTCACGTTTTCATTGCTGAGAGACGTTCAAAGCATCATCATTTCCAAAGCAA
AGGATGGGCTTTGCCTTTGCCACTGGTGAGGAGAATGTCACGTGCAGAATGTTAAAGTCTTCTTTAACAGTAAAAGCTTCTACTCAA
TACCAGCCAACGTGTGCTCTCCGAAGCCTCTCTAAGACAGGATGTGTAAGATACAGATGCTGGTCAGGGCCTCGTAAGATCTGGGGT
TGTAGGCTGCTGGGGGTAGGCAGTCTCGGGGATGCAGTGTGGAATGCTGGGTATGTATTGTGGAGCTCTGGAGTTCATACAACTCCT
CTGCCTCATAGAGGGATGGTACTTCCCTCTAGTTGGTAGTTGATCTGGAAATGTGTGTATGTGTTGCCAAATCTAACCATCTTCCAA
TAGTAGCTGGAACTCCACATACTTTCAGTTCAGAATCTATTAATTTTAAATCTCAAGAAGAAAGAAAATTCAAATTAAAACAAACAT
TAGACAGCCCTGGGGGTGGAGGTTGACTTTGGAACCTTACTGTATCAGATCTGTGGCCTGTCCTGTTGGCACCATGGTCACGTGCCT
GCTTTCATTGTCCTTTGAAGACTTGGCGATACTCTGTGCAGCTATGGCTTCCCATGACTGCCTGCAAGTTCATTTTTTTTCCCCCTG
AAAAATGTAAGTGTTCTCTTCCACCGGATAGAAGACTGGGGGGCCTTTCCTGAGTCTCAGTTCAAGTGAGTAATCACTCGGGATGTT
TCTCTCGTGCGTTTGTGCTTTTCTGTTCTGCTAGGTAACACATACAGCTTGAGTTAAGGGGTCGTTATCTGGGCCTGCTCTACATTA
CACAGGGATTCTTATAGCTGTGGTGTTTGTTGTAATGAGATTTGACCTCCCAGGGGAGCTGTAGATGCTTGGGCGAGTGTTCAGTCC
TTAATATTGGCATCTAATTCTAGGCATGTTGCCTGCAACTTCCAGTTTCAAAAAAAAAAAAATCTATGTCAATCTGTTCTTTTATAA
CCTAGTGCAATTGCTTCCCCCCTTTTCCCCCCAACACCCTGTTTTATATTTAATCATTCTCTGCGGCTCGCAACACCAGACCAGTCC
AAAGATTTACTTTTTCTATTTCCAACCCCATCATTAATTTTATGTAAAAAGTATGTTTTGAGTTAGAGCAATTGGTGGGGATCAGGC
TTGGACTCTGTGGGGAGAGGAGAGATGTCATTTACAACAGAGTTTATACAGATGAAATGTTCGCTACGTGCTGAGTTCCCACTGTAA
TCCAGGGGGTTTGTTTGCATTTACACTGAAAGAAAAAAGATGACCATATTTGGGCTGACTCTAAGCCAACATGATATAAGCCCCAAA
GTCTTTCTTGGAGGCCGGGGTGGGGTGGGGGGCAGGGAGCAAGGTGAATAGAAACATTTGGAAGCAATAGTTTGTTTCCCTGTTTTC
TTTGCATTTCATTCAGAATATTTCATGCGTGACTCAAGTTTTATAGCCTCCTTTTTTTTTTTCTTTTGGAAACAATGTCAAACCGAG
AGGGAAGTTTCAGAGAAAAACGGAGCTCTGTGTCCCTCCGTATGACTCGACTTCCCAGGTATGTCCTCACGGTGGCCGGTGCGCACC
TCACACTCCCATCTCCTGCCTTACCTTGGTTTTTGTGAATCCTTCCTTCCTTCATCTCTCATGCCAGTGACTGTTTTGATTTCTGAG
GTGAGGCCTTAGTCTTCAGCCCAGGCTGGGTTGCAGCTCCCTGCAGCCTTCCTCAGCCCCTTAAGTGCTGGTTTATGGTATGAGTCA
CCATACCTGGCCTTGGTTGTGACATCTTTTTTTTTTTTTTTCTTTTGGATGATGATGGTGTCAGTTGATGGCACAGAATATCCTTCC
TTTCCTCAGAGGATGTTCTCTGTTCTCTTCCCGTCCTTCACTACTGGCTGAGCTTGTGTTCCATTCAGTGGGAAAGGCAGGGTGTGT
TCATGAAGACCTGGAGTTTCTCGCCTCTGGATTACTACAGTGATTCCTTAGTCAGATATTCTTTATATGGGTATTTATTTATGTGAT
GTTTATTCTTTCACCTAGTCAAAACACACACACACACACACACACACACAGACACATGAGTTTTGAATTCATGGGTTCAATTAATGG
ATCAAAATATTTTGAAAAACGTTTATAGCAGGGTGAATGGCAAGTACCTTTAGTCCCAGCTCTGGGAGGCAGAGGTCATTGGATCTC
TATTACTTTGAGGCCAGCCTGGTCTAAATATTGAGCTCCTGGCCACCCGGGACTCCATAGTAAGGCCCTGTCTCAGAATGCACACTC
ATATTTATTGATTATTTACAGACCGTGTGTTATATCATTTCCTAAACAGCATGATGTAGTGGATATTTCCATCCAATTTACATTTCC
TGGGAATAATACATAATCTAGAGAAGATCTAAAGCGTATAACGGATACACTTAGGTCCTGTGCAAACAAGATACCATTTTATGGAAG
AGGCATAACTATCCAAGGACTTGGTGTTCTGGGGAGCCCCAGAACCAGTTCTGTGAGGAAGCTGAGGGACAGCTGTGCTCTGGAACT
CCTGTGTGCCACGTGGCCTATACTCAGGAGGAAAGCTGGTTAGTAATAAAATCCTGCCCTCGTGCAGCTTAGGTTTTAATGGGGCTA
ACAAATGAATAAGTCGTAAATAAGGTAGATCATGATAAATGCTGGAAGAGAGTCAAGTCAGGGAGGGGAGGAGGGAAGGGAGGAGGG
AGGGGAGAACAGACAGAGACCGATGTTCTTGATAAGAGGGCAGGGAAGGTCTCCCATGAATTTGATTATGGAGCAAGGATCCCAAGG
GATAAGGCCTGGGCAGGGAAGCCTCTGGAGTGAAGGCTTGTTGAGAGGCATCCTGGTAGCTGGAGCCTGGAGGGAAGAGAAAGGTGT
TGTTCCAGTGTTGATGTAAGGGAGATGAGAGACCCTGGGAGAGTTTTGATCCCGGAAGCTGTAAGATCTGACTTGTTGTGCCTTACA
GAGAACACCTTTTGTTATGTAGAGCAGAGCTGGTGAGGGGCAAGGTTAAAGGCAGAAGAGAGAAGAATTAAAGCAGGGTGGGGTAAA
CTGCATAGTGGCATGTCCTCTTGGGACATCTGAGGCACTCAGGGGCCATCAGACTTCTTCATCATCACCACCACCACCATCATCATC
ACCACCACCACCATCTCCACTATCATTACCATCACCATCATCACCACCATCTTTTTTGAGTGTGCACCACCATCCTTGGCTGAGACT
CGAACCTAGAACCAGGCAAACATGCGGCTCTCCAGTTCTTCCCGCCTCTCTCAAAGTTCAGAGCACACCGGGCTTTCCTGGGCTATC
TGTAAGCAGTGCTGCTGGCTGCTTTGCCAGGAACCTGGAAGTCTTTCGGTCTCACCTGTGTTAGTGTGAGAGCGGTCCTTGAAGCTT
ATGTTGATAGACACAGGAGCGAGTTGTTTTGGGATGAGTGAGTGATTGGGTTCTGGAAAGTTCTTTAGAGACACTTAGCTATGAAGT
GTTTGCACTCTTACTGTTTTCGTTTTCCAAGAACACATTTCTTAGCTTAAAAAATGTGGTCCTATGAATTTTCTCTCAATATTTATC
TGGTACAGAAGGCTATCACTAGGCACAGCATTTTTTGGTTCTTTTTTGAGAGAGAGAAGTTACGTATTTTATGAACTGAACACAAGA
CTCTGACGTCGTCGTTTTCTTTTTCTTCCCCTGGACACTTGGTTTTCAGGCAGAATAAATAAATGCTACCAACAGGACCCACCCACA
GAGAAGCTATTAAATGTTTCAGTAGGAAAGTCGGACTCAGGGGGTAGGTTAAATGAAGCTATTAAAGATGGATAAGAGAGAGAACAT
GATGTTGGGCCCAGTGGGCCAGGTTGCCTGCCCCTTGCATTACATCAGAACCTCAAGGAAGGAGAGGGTTTGGCCCTCTGACCAAAG
GCTGTTAGGGCCAGCTTTGAGGCACATTTGGTTCTTTTGTGAATTAACTTCCATTTTGCCTGAATAATGTTGGGTCAATTAAGAAAA
AAATGAGCCGCGCTTTACCCGGGCGATGCTAAGGTCAAGGGTACACATCCTTTGGAACCGAACTGGATTTGAGTCCCGTTCTGGTGG
GCAAGGCGTGTTACCTTGGCCTGCCTCCAAGGGAAGAACAATCAGTACCCGCTTCCTAAGTTCACTGAGTTAACGTCCGTGCGGCAC
CGTCGCGTCTGACATAAACCCTCAGCAATTAGCCTTTCCTCCTCTCCTTCCTGCTAGTACCATTGTTACCGTCATGAGCATTTTTCA
GTGACTTCAAGTCTATATCTGCTTTCTGGAGTACAGAACACTCTCAGTTATTGCTCTTTGGGGTAAGGGTAGCCTGCGTTGGTCATG
CCTGTTGTATTCCTGCATCCTCTGGTCACAGGAGGTGTGCAGTGTTTGGTATTGTTAATCCCGTGTTTACGGCCAGCCATGGCTGGA
AGCAGCCTGCTGCCCGTGTACGTCTTTTGCCTTTGTCTCTCCCTGTCAAAGAGGGATGTTCCAACTTCCTTCAGGGACATGTGGTGA
AAATTAATGAATAACTTAATGTTTGCGACTCTAGTCCATTAAGTACTAATGAGCATCCGTTACTATGCTGACAGTCTCTGAGCGCTA
GCATCGAACTGCTCTCCGAAGCAGGGGCATTGGTTGAAAGGGCTTTCCGTTATCTGGTGTGGGGGAAGCTTTTAAAACATGTCCCAT
TGCTTTATGTGACTACCGTTCTCCATACCCCAAATTCTAGACCTCCCTTGCCGGAGAAGAAAGAACTCTAATGGAGCAACTTCTCCA
AGGGTTCCGGTAAGAAAGTGCTGTCTCACGGTAATTAATTATGTTTGGGGACCAGCGCCTTTCACTTTTGACACTTACACTTGGTCA
TCTTATGTGAACTGCTTGTGTCTGAACTCCCCAACAGCCTTGTAAATTTCTTGTCACAGATTTATGATGGTCTTGGGTAGCCAAGAC
TCTGTGTGTGTGTGTGTGTGTGTGTGTGTGTGTGTACACCTATTAAGTTTGTATTAAGTGTCGTATTAAGTCTTTGTATTAAGCTTG
TGCTGTAGAGGCAAGATTAGGAGATGTTAATATCTACTATTTGGGGATAGCTATTATTAAATGTAGCCAATACAGTGATTTTATTAT
TATTATCTTAGTGTGACTCTATTTGATGATCTATGCAGCCAAAGAAGGTGTTTCTTTTGGGTCTTAGTATTGTAATGAGATTTTTGC
CCAACGTAGAGTTATCTTAAATAACTGATTTCCAGTGGACACCTCTTCCACCCATTGATGAGATCTTTTAGAGAATGTGAATGTATG
TGTGATATTCAAATTAGGTAATTTAGTGGGCTGCTGTTTTACTCTTCCGGGTTTGGCATGGACATTGTACCACTAGGGAAGTACAGG
GTAGCTCTGGATGGGTCTGGTTCCTGAGAGGTCCCAGGACTGGAACCCATGAGGCCCCCTCCCTGGACTAAAGCTGATTCCTGCTTT
CTTCTCGAAGCAGTGTTTGTTTAATCCCCAATGTTCCCATGATTACTAAATTACAATTCAATCTCTTGAATCTAAAGGATTAGTTTA
CTCTTGATTGATGCATAAAACAAGTTTTCTAATTTCTGGAACTGTGTCTAAGGAAAACCTCTGAACTGTAATTTTGCGAAAGGACAG
AGGATGTTGTTATAGATTTGGTCTTAGGAGCCCCGTCTTTCGACTGGACGCTGTAATTAAGTAACCTGCTCATCAGTGGGGAGGGTG
ATAATAGTACCTGCGCTAATCTCTGCTAAGCTCAGTGATCCTTTGAAGTGGGGTCCAGCTGTCTCCTCCCGGTTTCAGGTCCTCGGG
GAGAAACAACAGAACTCGCTAAGGACCCGAGAGGACCCTGTGCACGCAGACTCACCAGGAAAAGACAGTTTGAGGGTGGATAGGGTT
GGCAGCTGCACTGAGGCGTGTTTGTTCGCCAGTTCTGATAGCAGGTCAAAGTAGTTTAACCTCTTACACAGCAGTGAGAGGCACCAC
TTCCCATTCTGGCATCACTGACCTCCCTCTGAAAAGGGACAGGGAAACATAGAGGAACGGGAGGGCGCTTATCTTTCAGGTATCAGA
ATTCAGACCAGTTTCAGATCGCCTGGTTTTTAGTAGTTCCATGACTCTCGTTGTTGCTGTTTCCCTCTACTCCAGCACTCCGTTTTG
GAGCTCCTAAATGCTTAGGTCAAGCATGCTCACCCACCCCAATTCTTATTTTTGTTTGAGACATTTTTTTTTTCCTACCGAATCCCG
GATGACCTCATTTCTAAGCCAGAGTAACTGTCTTACCCAGCACACCCATTGAATGTCTTAAAAGGCTTTTCAGATCCATCCATATAA
AACCCTCCACTTGCTCCTTCATGGAAGCCGACTGCTGTGGCCTCACACAAGCACAGCTGTGCTGGTGGGGTTGGAAAAGGTGTCGTC
ATTCTAGGTCCATTTAGACAGGCTTCTGAACAGCTAGACAGACATACAATTACTGTAAGAACCAGCACTCCATGTGTATATTCCCAA
GAGAATGGAAAAAGCAGGCTTCAGATGAAATGTATACATGTGTGTTCAGTGTCAGCATTATTCCAAGTATTGCAAAAGGAAAGGAAA
AAAAAATCCCAGCTGTCCATCAGTGATGAGTCTGCATAAACAAATTGTGATATTCTCGTACAGTGCAATGAAAAGGAGACAGTAATA
GTGTATGCCACAACATGAATGTACTTAGAAAAAGTTATAGTAAATTATAGCAGCCATCTGCAAAAGGATGCAAACCCATAGAAACAG
GGAGAATATCAGGCAGGGGCAGGGGGTAGCGGAGAGAGACAGGGAGTGTATATCAGAGTGCAGGGGAGAGAGACAGAGTATATCAGA
GTGCAGGGGAGAGAGACAGGGAGAGTATATCAGAGTGCAGGGGAGAGAGACAGAGTATAACAGAGTGCAGGGGAGAGAGACAGGGAA
TGTATATCAGAGTGCAGGGGAGAGAGACGGAGAGTGTCTCAGGGGGCAGGAGAGAGAGACAGAGTATATCAGAGTGCAGGAGAGAGG
AATGGGAGTTTTTTGGAGGGATTTGAAAATGTTTTATCATTGGAAAGATATATTGTGAAGCTACCAAAATCTATGAGTTGGCCACTT
AAAAATAGTCTAAGTGTTGATTACAGGAGTCCCCAGGGACAGCTGCAATAAAGTGACACAGGCTGTGAGGCAGGAACAACAGAAATC
TGTGTCTTACCAGTTTGAGAGGTGGGAAGGTGGAGGTGGAGGTGGAGGTGGAAGAGGCCAGATTCCCCTTAAGGACTGAGGGTAGGC
TGTTCCAGGTCTCTCTGCTGCTCCTGGTGTGAACACTCAGGCAGACACTGGGTTGGAGACCCTTCACCCCATCTCTGGCTTCATTGT
CATGTGACATTCTCCCCGTGTGTGTGTGTGTTTGTCAAGATTCCCCTTCTCATTAGGACATTGTCATAGTGGAGTAGTGTTCACCCT
GGGTGACTTCAATTTAACCAATTATACCTGCATTGACTCTGTTCCCAAATGCACTGTGGAGTGCTGCGGTATCACTTTAACATATGA
CTTGGGAGGGGGTATTCAATTTAGTCTATAATGGTGTCTATTATCTAGTGTGATTTACTTCAATTTTTTTAAAAAAGAAAAGTGGCT
GCAGCGATGGCTTGGCAGTTAAAGAGAACTCGTTGCTTACAGAGGACCCACATTTGATTCCCAGCGCATAGAACTCCGGTCCCAGGG
GATTCAACACTCTCCTCTGGCCTCCTGGGCACCAAGCATGCAAGCAATACATAATCACACATGTGGGCAAAGCCCCAACACACACAC
ACACACACACACGCACACACACACACACACACACACACACACACACACACAGTGACAGCGCAAGTTCAAAGTCAAAGAGCAAAAATA
AGAAGGACGTAAAGACATAAAGACATAGAAAACAGTTGAACCCTCCCTCCGTGAACTTCAGACATCTGAGCGGCTCAACCCCTTATG
GTCTGACATCATGGCCTCTGCTTGCCTGATTCCTCTGCCAGGCAGCTGGTTTGTGTCCACAGCCTGATTCGCTGTTCCGACTGATGG
AAGTCTCTTCACCCTTAGAGTGCAGGCCCAGTGTCACACTGCAGGATACTGTCCCCTGTGCGACCTCTCAGTGTAAGACTGCTGCCT
CTTGGTCACATGCAGCACTGTGTGGGTGACCTGCCACCGTGTGACGAAGCACGGGTCAAACAGCCACCTTTCCTTTACAGATTGTGT
GGGTCAGGAGGCCAGGCAGGGCAGTTGGGTGACCCCTCTCTCTGGTATAGAGCCTTGATGGGAAGATGAGGACACTGGGGAGGGGGC
GGGGGCAGTTCCAGGTGTGTGTCTGCACTGGCAGGACTTGAGGTGACACTCAGCACTTCTGCCCTGGGGCCTACGTGTAGCTGAGAT
GCCTTCACAGCAGTCGGACTTCAGGGTGTTCACACTTCTCTCAGGGCTTTTAGAGCTCCAAGAACCAGAATTCCAGGCACGAAAATA
AGCAAGAAAATGCATGGCCTCTTCAATCCTAGCCTCAAAGTCACATGACATCATTTCCACCATAGGCCTGCCCAGATGCACAAAGAA
TGGCCTTAGGTCTCAGTTCTCCGTGAACAGAGAGTCTGAGGCCGCCTTTATTAACACTCAGTGCTACCTTGCCCCAGAGTCTGAAGC
CCCTCCCCCACGAAGTCATATGTTGAGACCTATTCCCCACTCCTATAATACTGTACCCTGGGGCTTTGATGATACGGGATGCCTGCC
CCCAATAAAGGAAGCCCAGGGTACCATCTAAGAAGTGGCGAGCGGTTTCACATCCTCAGGTCCTGTGACCCTGGATCTCTCAGCCTC
CAGACTGTGAGACGAAATGTCTGTTGGTTTTTAGTTACTCCTCCCTGGTAATTTCACTTAGCAGTCACCTGACAGACAGCCGCTAAT
CAATCTGTGCTCTGTCAGGTCGCTCCTTGGTAAACCGCTCACTTCTTTGCATCATTATTTAATTGATTTTTGCTTTTCCAGTGAGAG
GGTAAGCTGGTTTGTTGTTGTTGTTGTTGTTGTTGTTGTTGTTGTTGTTGTTTTCTGTTGGATCCTGAGCTTCTTGGTGGCTGGCAC
AGAGAGCAGGAGCTTGGTGAACTGATGGAAGGTGAAAAACATAGAACCAGAAAAGCCATAAGCATGATAAACCTGTAAGGCAGGCTC
AGTATGTGTGACCCACGGCTTGGTTGGATCATCTTCCTGGTCTTGATTCTGCAGTCAATCTGAGGCTGCCTCGTGAGCTGCGGAGTC
TTGTTGACGCCCGCCATTAGACACAGCAACGTAAGTAAAAGTGAGGCCGGCTGTTTGCTGAGTGCTTTCTACAAAAGCGGATAAACA
GAATTAAAAATTTAAGGTTTAAATAGGCCACCGCTAATAGGGCTCATGCGCTCATGGATTTGTGTTTATTGAGTAGAGGGAAGGTAG
GATGTGGTACAGTTTTTTAGTTTTAACAGCCTGCCTTTGTCAGAGCTTTATGGGGCTGATCATGTTTATTTAACACTCCCTGTGGGG
CTTAGCATGAGAGTCTCTCATCTGTTGGGCTTCTCATTTCTTTTAAAAATTCTACTTGGAGGTAGAGGCGAGTGTAACCCTGCAAGA
CATCGTCCGCTTCTTCCCCGGTTTCTGCACAGCTGCCTGACAGCTGCCTTGGACTTGTTAGGTGAGTGCCAGTCTCCTTTTTTTTTT
TTTTTTTTTTTTTTTTTCCTGTAGAATTCCAAGGCAGGGTTTGCACAGGACCCCTTAAAAAATGCAGGGTGAGGCGATTCTGTGATC
GAATGTGCAGTTTTTGTTTTCTCCTATGCAGCAAAGACCCCCACCCAACTCCCCAAGGCAGTGCAGAGCAGAGACAAAAAGCACTGA
CTACAGAATTGCTTGATCCCAAGTGTGAACTCTGACCCTATCACCTTTTATTGGTGTAACCTCAGGCGAGTTAGGCACCTTCCATAT
GTCTTCCCTGAGCCCTCCATGACAAGGAAGGGGGATGTCTGTGAAGATCCAACCGCAGCCTGTCGTGTGGAGAATGTTTGGGGGAGT
CCTTGAATGGCTTTTAAACACTGGTCTTATTCTGGCTTTGTTCCCATTGGAAGGAAAAGCCTATGCTGTTTTAGGCTTGGAATATTA
GTATATAACTGAGCTCCTTGAGGATTTTGCTTTGAAAAGCTTCTATTAAAGAATAAACATGGGCCGGGAGATGTTGGCTCAGTGGTT
AAAGTACGTACTGCTTAACCATAAGGACTAGAGTTTAAATCCTTAGCACCCATGACAGAAAGCCATAGGCATGGTCACTGCCTATAA
TCCCAGCACTCAGTAGGCAGAGATGGGTTCCCTGGAGCAAACCAGTGAGTGAGACATGTCAAATCCATGAGCTCTGCATTCGATTGA
GAGAGCCTGCCTCATAAATAAAGTGGAGAGCAATCGAGGAAGGGCCTTCATATGTACGCCCTTCCCACCCCACCGCCCATATACACA
GGCACACCACAGAATGAGGACGTTTGGAACCCGAATGTACTTTTCTACATTTCTCTACCATCTGTGACAGCTAGCTCATGCTGCCAG
GTAGAATGTGTGGACTTGTTTGTTTCCTAACATTGGATTTAAAGTTGATGGCTCTTCCCCTTCAGGGCCAGGTCTGAGTGAGCCCTG
TGGACGAGAGCCCCATACCCTCTGTTCAGAAGACCAAAGATGGCGGCAGATGCTGTGGCTGGTGAAGTGACGTCAGAGGAGGGAGGA
GGGACGAGGGACGAGGGCTTTATAACAGATGGTTTCTTGGATATCTGGGGCTTGTCCAGTTCTAAGCTTTGAAAACGATGGGCCAGA
CCCTGAACTGGCACAGACCCAGGCATCGTTGCCAGAACAGGAAGTGAGGTCCTAGAGTGATGTCTTTGGGACAGCTGCTGCTGCTCA
GCTGGTGGGCTGGCACAGTTGGCATTGGCTTCCTGGGGTTTAGTGAGGTGAGTGTGTGTGTGTGTGCGCGCGCGCGCTACAATGGCA
ACAACGAGATGGGAAGCTGAGAGGGTCGTGACTGAGTGGGGTTATCCTCTGATTTACGAGAGGTGTCAGGTGCGGTACCATGTGATA
GTTTTGCCGACGTTGGCATAGATGGAAAGGTGTGACTGTAGAAACCAAAGATCCGTGCATCCCACGCTCATCAGCCTGGAGTTCAGG
CTTGTGTGGAGCACAGAGTGATGCTCTGTGGGAGGGCTTGGCTGTCCTCTTGACATTAGCCTCCAAAGACTAGGAGCCTATCCCAGA
CATTGCTAACATCCTCTAATACCCTCTTGGAGTAGTTAAACCATTCTTGGCTGGATTCTAGTATTTATCCTGCCTCTCATTAGCGGC
ATGTCCTTAGGCAAATTATTTAATCTCTCTATGCCTGGCTAGTTTTGTCTATACAATGAAAACCTGGACTGGATGATACATTGCTTA
TGTCCAAATATAATTTAATAATATTTGATTTGTAAAAAATATATTTTAGTGTTTTCTTTTAAATGTGAGCTTCATGAATTTTGTTTT
AAACACACACCTTTCTTAATTTTTTTTCTCTCTTAAGGATTTAGAGAGTGCTTTTCAATAACTAGTGTTGACGTGTTTGGGAATTAG
AGTTTACTATCATCATGTTAAGGATCTATATTTTCCCTGTTTCTGTTTCTATATTGTCTTCTTCCTTTCATTGTTGGGAATAACACT
AGTTGCTATCCCTCTTGGAGAGCAAAGGCCCAGAAAACAGGCCAGAGAACCATCAGGGCTTTTCCCTGGAACTTAGAGGCTACCTGG
TCAGTCGTGATGCTGAGAAGAGGGAGATACTGAGGCCCTAGAAGGTTTGTTTTTTTTTTTGTGAACAAGACTGTAAAGCATAGACTT
CTTGACATCTTGACTCTTTAGCTTGCTTTTCTCTGCTGTTTCGTGGCATTTGGCAGAGTGATCTGAAAACGATCTTTCACACCGGCT
CTCACCTGACAGTAGGAATGGTGTCTGGTCCTCACCCAGGTCCAGCAGCATTAGGGAGGGCTGCTGTCTGGTCCTCACCCAAATACA
GTAGCATCATTTTAAGGTAGAACTAACTTAGGGGCTCCAGGTTCCTCTTGAGCATGCTACCTCTCAGGGACCGAGCTCAGGGCTTAG
GTGTCTTGACATGTGAGTGGGGAATAGGGGGCATTCTATGGAGTGTGAGAAAGCCGGGCTAAGGGACTTGTGTTCACATGAAGCTCA
GAGACCATGGTGCAGTCCTTGCTCACATAACTGGTGTCAGTAGATGGCTTGCTGGCCAAGTTCTGTGGAGTTGGAAACTGTACTGGC
TGGTTTCATCTGTCAACTCTGACACAGGCTGGGGTTATCAGAGAAAGGAGCTTCAGTTGGGGAAGTGCCTCCATGAGATCCAGCTGT
GGGATATTTTCTCAATTAGTGATCAAGGGGGAAAGGCCCCTTGTGGGTGGGACCATCTCTGGGCTGGCAGTCTTGGGTTCTATAAGA
GAGCAGGCTGAGCAAGCCAGGGGAAGCAAGCCAGTAAGGAACATCCCTCCATGGCCTCTGCATCAGCTCCTGCTTCCCGACCTGCTT
GAGTTCTAGTCCTGACTTCCTTCAGTGATGAACAGCAATGTGGAAGTGTAAGCCAAATAAACCCTTTTTTCCCCAACTTGCTTCTTG
GTCATGATGCTTGTGCAGGAATAGAAACCCTGACTAAGACAGAAAGCTTCGAAGAGGTTGATGTGAGTCCCGGAAACTGGGTTCCTG
CTTGCAAGTAACCCTTCTGCTTCGCAGCCTTCCTGCACCCTCCCAACACCCCGTAACAGTTCACACAGCTCTGCAGCCAGCCCCATT
CCGTTGTTTCTCCTTCAGCCTCTTCTTCCTCACTGGACATTTTCTTCATGTTAAAACATGAGTTTATTAGTAAATTTAAATGTCTGA
AATTCCAGCTACCTCAGTGCCCTTGTTAGCCTAGACTTGACACAGCCCAGAGTCAATATGAGTCACTAACTGGCTTTGTCAGTTTGG
TCTGTGGACAAGACTGTCAGGGTTTGTGTCATGATTATTAATTAATGCGGAGAGTTTGGGATGGCCCAGCCCACTATGAGCATCACC
ATCCCTAGGCAGGTGGTCCTGTCGACATGAGTAGAGCTGGCACAGCATGGGCCTGAGAACAAGCTGGTAAGTACCATCCGCCATGAA
ATCTGCTTCAGATACCTGCCTTGGATTCCAGCCATGATCTCCATCATGATGGACCCTACCTGGAAGTGTAAGATGAAATAAATTCCA
TTCCTCCCCJGTTGCTTTTTGTCTGACTCTGTTTTATCACCATAACAGAAAGGAAACTAGAATATTTAAAGAATCAGTTTGTCTATT
TGATGTAGTCATTTTCTTTTCACTGAAACCACTGTTAGGAAGCCTTCCGAAAATCTGAAAGACCAATAGGTTGAAGATGAGATTCTG
TCAACAGGTGGAAGGTGAGGAATTCAGAGAGGGAGAGGAGAGAGAGGCCAGCGTTTGCCATACAGGACAATTTAAGTGAGGGAGAGA
CTCACTGTGTTATCAGGTTCCACATCTGAGTATTCTGATAATTCCATCATACTGATAATGCGATCATTCTGATAATACTATAATAAC
ATCCCTGATAACTGCAGATTTTATTTTAATTTCTGTTTTTAATTTGTATCTGTTCTGGACACACATAAGdTTCTTTCTGGGTCCTGT
AGTATACAGAACTGTACTGCAGCTATTGCAGGAGTGATGGCTCTTCATTGAGTAAATAACCTAGAGATGATTTCAAGTCTACCAGGG
GATATGTAGAGCTTATATGCAAACCATGACGTTCCGTTCAAAGGACTTGAACACTAGAGGGTCCTGGAGTCAGACCCTCACAGCTGT
CCAGGGACGATTGACCCGAGGGTTTCAGGCTGGTGTAACTTACTCAACAGCAGCCTTTCAAAGATTGGTGAGAAAGTTTCGACAGTA
GCTAGGTTTCAAGGGGCACAGACTGCCCAGCAGGGATGCTGTTGTTGCAGATTGCACATGGCAAAGGATAGGAGCCTTGAGGGTAGC
TAAGGATAGGAAGGGTGGTTCTTGGCTAGGTCTCATGGATACTGGACTGGCAGATGTTTCTAGATCCACAGCAATACTGGGAAATAG
TTTATGACTAATAATTGATCAAAGTCTCTGCCAAGCTGACGGTGATGGGCCAGGAACCATCATTTCTGTGTATGTGGAACTTGAGGT
ACATGGTTACGGGCTGTTGGGCATCAAGGTGTCGTCTGCATATGCCTGTGGTCGGAGAGAACAGGTGGAAGTGGGCTGTGGGGAAGC
TGGAGCTCAGACCTCACGCCCACGTGCTTTCCCATGATCCTCCCGACATTTAGTCACAGAGACAAGCTGGCAGGATGAAACGAATGG
TAGCATTCATCATCCAAATTTAAATTTGTTTCTCATTAACAGACATTCTGCCTGTAGTGCACCATGAATTAATATGTTTTATAACTC
TTGTAACTGTCTCGTGGAGGGCATAATTAGTCATGAGTGTTTGGGTCTTAGAAATGTTAATCTGTTCAATGTAGGCATTGAAATATT
GCGTCGTTTCTTTATTTTTTAACATCTCTGAGCGCTCTAAGAAGATGAGAGTGGCCGTGGCCTCTCTTGACCTCTGGGCGGCCTGCT
GAGCCGCCTCTGCAATTGGCTGGCCCGGGTCATGTGCTCACTTGTGGTCCAATCAGCTGGGATGGAGGCATCAGTGTATAGAAACCT
TTGACCCGGGGTTGTGAATCAGCTTCTGCCTTCTGGAAATGCTACCCAAGAGCACAGGGGGTGTGCAGAGGGAGGGAGGNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNACAGCAGAATTCTGATTCAGTCCCGAGGCTTTGAAGTTTCGAACTCCGTTCCTTTAATGCA
TGCGTAGAGTGCGGGTAGATCACGCAATCCCTGGGGACCCTGCCAATCACGTCCATCCGAAAGATCAAGCCACTGCTAAGCTGCTTC
CTTAGAGTGAAGTGATTGTTCAGGCTGTTGGTTGTTGTCTCAGAAACGCTACAGAATAGACAACTATAAGGGGAGGGAAAAAAAAAA
AAAAGCAAAACAATTCCAGAAACGAGGAGCGGATAAAGAGATGCAAGGGGCCGTTTAGTCAGCTAGATGAAGACTGGAAGTGATAAT
TGGCGGTGTTTAGGGAGGGTTAAAATTGGAAACCAACGCGAAAACGCAGTTTAAGCCACTGTGTGTGTGTGTGGGGGGGGGGGGGGT
TCAAAGTAAACACTTGATATTTGGTAGTTACAGACACACAATTTTAAATCGACACAGCACCCTCCAGTGCTCTATTCCCCCAAGTTT
CTCTTTATAACTTGTAATAATTTATTCCACAACCACCCTCAGAAGATAGCTTAGGCTGGGCTGGACCGCATCCGATACCATGTTTGT
TGTTTGGCCAAAACTTTGATGTTTACGGAAGGGGACCAGAGGCCTTTATAGGAAAAAACACAAAAAACCTGGCGTGTTGGCAGATGT
TGAAGTAAGCAGTCCTGGACCCTGCTGTGTTATGAAGGGAGGGATTTTGGACCTCAGTGTTCTGCAGACACTACATTTGCGTTATTA
GAAAACACAGCACCATGGATAAAATGTTGGCAGCTCCTACAATAGCCTGGCAATGTGGTTTTCTTGTTCATTCACAAAGTCTCCTTG
TGCTTACTGAGACCAGAACGCACCGCACCGGGTGGGTGGGGCTTACTCCTGGACACCTTGAGATGCCTCAGGAGGGTGGAGCACAGG
CATGGCCTGAGAACCTGCTTTGCTTCTCTGTGTCCTGACAGTGCTCACCAGAAATTGTAACATTATTGTGGATTTTTCTTGAGACCC
TTTCTATCGAGCTATTTACTGGACCTTGTTATTATATTTCAGTATATTATTTTGCCAGGAAAATGTCAAGTCCATCGACTCACTCAT
GCAGCTCAAAACGAAGCAGGTTTTGGGCCTCCTCCTGACCAGTTGAGACTGAACCCTCCATGCTGGGCCTAGGCCCTATGCTGCAGT
GTCTGTATGCAGTGCGTATTTGTGGAGGCCATAGAGCAGCATCCTGTGTCACCCCTTATATATTTTTGGAGACAAGGGTCTCTCACT
GAACATGAAGCTCACAATCTGCAAGACTAGCTGACTGGTCAGCAAGCTCCAGAGGGTCTTGTCTCCACCTTTCCAGTGCTAGGATGA
CAACATTTGGATCTATCATGGTTGTTCTCACCTGGAAACTGGGGATTTGAAGTCTGGTCCTTATGATTACAGCAAACATTTGACTGA
CTAAACCCTGAGTACTGAACCTCAAATTCTTTTGTCTCTTCTATTTTCCTGTGTTCTTTAAGTGACTGAATCCTGAGGTTGGTGGTT
GTCACTGTCACTAAAATGTTGGAACAAAAAAAAAAGACAAACCAATTGTGGTGGTTTGTATATGCTTGGCCCAGGGAGTGGCACTAT
TAGGAGGTGTGGCCCTGTTGGTGTAGGCGTGGCACTGTGGTGGTGGGCTTTAAGACCCTCTTCCCAGCTGCCCAGAAGCCAGTTTTT
TGCTAGCAGCCTTCAGATGAAGATGTAGAACTCTTAGCTCCTCCTGCACCATGCCTGCCTGGATGCTGCTATGCTCCCGCCTTGATG
ACAATGGACTGAACCTCTGAACCTGTAAGCCAGCCCCAGTTGAATGTTGTCCTTTATAAGAGTTGCATTGGTCATGGTGTCTGCTCA
CAGCCGTAAAACCCTAACTAAGACACCAGTTCTTGTGTGCATGGTGATATCTGTCCATCTGTCTTAGAGCTTGATAATAAATATGTA
AATACTTCAGAGGGAAGGAAAAGTCTCATAGTTATACATTAGAGTTATTGGTGCCAGGAAAACTAGGGCCCGGGAGTGGGGCAAGGT
GAGCGAGTGAGAGGAAGGAAGTCTAGGTGAGGAAGAAGATGCACTATAAACTGAGTGCTGCAGAAGAGCTAGGCGTGTGTGTGTGTG
TGTGTGTGTGTGTGTGTGTGTGTGTGTGTAGGCCAAGGCTGCTTTTACGTGGGACAGTGTCCCAGAGGGCCTGATGAAGGAAAGTGG
AGGTGGGGTGCTTGAAGAGCTAGGCTTATGGCTTTGAGTACCATGTGTGAAGGGACTGTGACCCTGGGAGAGGTAGCCTTGGTCACT
CTGTGCTGGTGTCTCCCCGGTTTCCTTGGTCCTGTGCTTTCATAGCGACTTGTCAGCTTTAGCTCTCTCCCTGCTAAGGAAAATCTC
TCGGCTGTTGGCTTTGTTTGAGGAGCTGTAAATGGACTTCACCTGAAACACATTACAGGCACCGGAAGATTAGTTTTAAGCAAAAGA
ATTTGATTGAAGGCCGCATCTTGTTCTTGCAAATCCTTTGATGTTTGGCCATTGTCTTGTGGGCTTTTCATTTTGCTCATTGGCAGC
TCCGTGCAGTGCTGGGCTGTAAAAGCCTTCACCTCCAGCTTGTCTTTGTGGAGTCTGGAGGTGGCTTCCTCCTCCCTCTCCCCCTCC
TGGGACAGCAGAAAGCTCACCCAGTTAATTCTTTATAACAAACTATTCTCTCTTTCCAGGGAAGGGGTTCTGGGGCATTTATTGGTA
GGTTGTGGTTATTCCCTGGTCTGGAGTCAGGATCCTAGGGAATCATTGGGTCGAGCCACATGCAGTATGGGACAGAGGTATGTGTGT
GACCTGGTCAGGAGGTTCTAGTGCTCTTAGCTGAAGACAACACAGGCAGAATCCTCAACAGTGGAGCCACTCACCACCCTCCAAAGG
TCCAGACCAGGACTTGGGGCTCGGTCAGCCCAGAAGAAGCGGGTGTCTTCTATTTGTTAGAAAACCACTCCAGCCACTCAGCCTGGA
GAGTTCTAGATAGCTCTGCCTGGGAGACAACCTCACTCAGTAAGCAGTGGGGATCCCAAGAAGGATCTGTTCTTACTTGGTGGAGTT
GGGGCTGGCAAGCAGTCTTTGCCTCCTGTGACAGAGGCATCCCTGAATAGAGTGGAGACTCAGATCATGGCAGAGTGAGTAGCCTGG
ACCTGGACGAGGAGCTGAGTCTTCCTACGGACTACCCTGATGACTACATTTGTCACGGTGTCTTAGCCAGTAATATAAGTGACTTGT
TTCTTTAAGAGAGTGACTTCAAGGTCAGGGGTATGGCTCTGTGGGGGAGCATTTGTTCCTTCTGTATAGGATCATGGGTTTGATGCT
CAATACTGTCCCTCTCCCCAACACACAGAGAGAGGGTGACCTCAAAGATAGGTGCCTCATGTCAAGTCATATCCTACAGATTACAGC
AGTATCCAGGGGCCTTGGCAAAAGTGCTCCTCCCCAGGGATCGGGGCTATATGCCAGGCTTCTTCACCTATGGGTGTTTCAAGAGCT
AATTAATTTGCGCTGACCTTGAACTCCTTGCCCAAAGCAGTCCCCCTGTCTCCACCTTGGAAGCCATACCAGGCAGCACTGAGGGAT
ATAGTTCTAAAAGGGTCAAGAAAACTCTGGGGGCCTAGCTGCCACATGGGTCCTTGCTCTTCTCTGACCTGACATCAAGGTGGCAAG
GGACCCCAGGTGAGGTGTTAGCCAATAAAGACAGAGTTTCAGGCCTGCCACTGTCATATTACCATTTGTTGACAACCGTCCTTCATT
AACCAACCTGTTCTTCACACATTGGTTCGAATGTGCTTCTCGGATTTACTTCATTCAGTTCACCAATATGCGGTAAGCACTGAGGGG
CCCAGTAATTGGAAGTGTCTGTCGATGGCCTCTGCCAACCTGGCTCATGATGAAAGTCCTACTTTTATAACTCAGTGTCAAGCTCAA
TAAGAGGGAGGCCGGTTCAGCTATTGTCAAATCTCATTGTTTTTCCTACTGGGAGGACCACAGTGGGTTCAACTGGAGACAGTTGCT
GTCTGAGCCCAAGGTCCTTCTTTGTAGCTGGGCTGAGGACTGCAGGTCTCTTTAGCCTGTACACATGGACAGAGGGCCCTCCTGCTC
TGCATACCAAGAAGCCATGCTGGCTGTCTCTCCCAAGCTCTTCTCTGGCCACTCTCCTTCCCCCCACATCTGGTGTTTGGAGGAGGT
TCTCCTTTCTGAGGGCAAAGGGTACATAGGGGCATTAGTCCCCTACTCGGAGAGGTGAAGGTAAATGGCGAAGAAATTGCCAGAAGC
ATCCTTTTAACATCCTCCGTGAGCAAAGCTCGGCCTGTGGAGTGGACTGGTACCTAGACTCCAAATCACGGAGGAGTGGCTACAGCT
GGAATCGGGATTGTCTTTCCTTGGCCACCTCTCTTGTCTCCAGCTGTCCACCATTGCTACTGAAAAATGGATTTTGAAGACACCCAT
GTTGGGTGACTGTGTGTACCCGGAAGCACCAGTGGAAAGGGGTACACAGACACCACATCTTAGTTTGCCTCCACAGCTGAACGGGCC
CTGCCTGCTTTAGTCTGGATTGTTTTTCTCTTCTGCAGCTCCAAGTGGAGTGAGCAGGACTGCTTTCATGTCCTGTAGCCTGAACCT
GCCCTGCTATGTCCCACTCTGCGTGGTGGTCATCTGTGGGGCTTGATCAGCTGCATTTCTAACTAAATGGTTTTTTTTTTTTTTTTT
TTTTTTTTAGTTGAAGTCATTGTGGCACCTGGACTTGAACATCTTGCTCTTGTGTTCATTGTTGTTTTTTTTCTCCCTCTCCATTCC
TGTCACCAGCCTGTAGACCACCTTGAATTCACTCCAACTTGCTGTATAAACCCCTCTAGCATTCCAGTGATAGAGAACTTTCTTAAA
AACAGAAATACACACAGTTTATCAATATCTGTGTTTTTTTCTCCCTGGAGGAGCAAAAAAAATCAAATCACCACCACCACCACCTCC
AAGAAACCAAAACCAACCAACCAACCAAGCACCAAACCAAACCAAAAACCAATTTAGATTCAGGAGCGGGAGAAAGGAAAGAAAATG
TTTTTGATGATGTTAAAGTTTTGGACTTAGAGGCAAAAGTCCTCGAACTATTAGAACCAGAAAACACTGCATACTGAATAATGTACA
CCCTGAATGCATACCTGTGGTTCAAAGGTGTGGGTCTAAATGGCTAGTTTCCTCTCTAGCAGCCCGGAGGTGTTGCATTCATTCCTT
TTGGAGAGAGACATATCACTTTTAAAAAGTGGCTAAAAATTGCTGGAGACCCATTTGGAGACAGGATATTTGGACTTCATTTGGCTG
TTGTCGCCAGCCAGTGTTGGGACCTGGTAATAAAGCCTGGGCTTTTAGCGGATCCCTGGGATAGGGAAAGAGCTAGACAGCATGGGC
CTGGGAAAACTGTGGGCCACCAAGACTCCATTTCAATGCGAACTTATTCTACCAAGAGCTGCTGTAGCAAGAGGGAGGAACCTAGAT
GGAGCAGGGGTCTCTGAATGCTGCAGGCTTACGTGTTAGCATCTGGAGGGCAAGAAACACCAAACTTTAGCAGAGAAGTTCAGTGAG
TTTAAAAACCATAGATTCAAGGAGCTTAAGGAAGAAAATAGTTCCTGTTGTATGGGTCCATATAGGTCCCTGTCACAGGTATCCTAG
GAGAGAGCGAATATAGGTCCCTGTCACGATATCCTTGGAGAAGAAAGGGCATATGGTTCCTTGTAACAGATATCCTTGGAGAGAGGG
TACATGGGTCCTCGTCACAGGTATTTGGTGATGGGGAACATATAGGTTCTTGTTACAGGTAGCCTTGTGTGTGTGTGGGTGGTGGTG
GTGGTGGTGGTGGTGTAAGGATTCATGTCACAAATAGCCGAGTGGAGAGACAAATGGGTTCCTGTCACAGGTATGGGGGAGAGACAC
ATTGGTTCCTGTCTCAGGTATCCTTGAGAGCAGACATGGGTTCCTGTTACCGGGATCTTTGGGGACTACATGGATTCATGTCACAGA
CATCCTTGTGGGTGTGGGTGTGTGTCTGCACCACAGTCTAGAATCCTGTGAGTTCAAACAGACGTGGTTCATTCACACTCCTGCAGC
GTGTCTCGCATGTTACGCTATAATGAATCAGGTTCCAAGTTAAAACTGTTTTTCGTGAAATCCTGTGGGGAAAAGTGTCAATTTATG
GATAGGTGCGGTGTATATAGATTTCATGTAGAGCATACCAAATGGTTTTCTTTTTTTAAAACCATGCGCTCTAATTACAGATGGAAA
GTGTCTAAGGAAAGTTGGACTAAAACAACACACACAGACGTATATATTATATATAAAGATGGACATGTGTAAATGATAAAATGCTGC
ATACAGGAAAGGGGAAGCTGCCTTAGACTATCAAACCCATTACAGATGAGCATGATACTTACATGAGAGCCTCCTGGAAACCAAGGC
AAAAAGAAAAACATGGTGGATGATAGGTCTCTAAGTATCCCTCCTCCTCCTTCTTCATCTTCATCCTCCCCCCCCCCTTTTTTTTTG
TACTGAAAAAAGAAAAGACCGAGAACCTTGTTACATTCCTTAGGTTGGCACAGATGTATTCTGTAGCCTAGGCTAGATTTGAACTTA
CCATCTGTCCAACTGACCGACCATCCTTCTATCCATCCATCCATCCATCCATTCATCCATCCATCCATCCATCCACGCACCCACCCT
TCCGTCCGTCCGTCTGTCCATCCATCCACCCACCCATCCATCCATCCACCCACCCATCCATCCGTCCATCCATCCATACACCCATCC
GTCCACCCACCCACCCATCCACCCACCCATCCATCCATCCATCCATCCATCCATCCATCCATCCTAACCTAATTAGCTGAGATTAGA
GGCCTGTACCACTAAGCCTTCCTAGAAATCTTTCTTCAAAAAACAAAACAAAACGCTACACACCTATGTCATGCCATGAGGTGAGAA
ACTCTGTTGCTAACCTCTGAGATTTTACAGCCTGTCGCTGATATGCTTTCATTCTGGCTGGCTTTACCAGACATCCAGAGGTACCAC
ATTCTCTGACAGCTTCCCAGCTGCAGTTGGGTTTTCATGGGAGTCCTGAGAGGTTCCTCTCCTGGGCCTCAGCTCTGGCTCCTCAGT
GAGCTTAGTTCTCTTCCCTTAGCTTTCAGACTTACTTGTGGGGCTGCCAGGAGCTGGACAGGCTGCACTGAGGAAATACAGGGAGCT
GGGGATGGTGGGGGTGATGGTTATTCTACTCAGAAAATGGCAGCTCAGAAGCATGTGATGGGTCTGACCGTGAAGGAGGGGCCCAGC
CTTCCTACCTGAGGGAGCTGAGCTGAGGGTGGGAAGCCTTGCTCCAGCATCCCCTTTTGCTTCTCCATGAAGTCCTGTGCCCGCCAT
CTTCCTCCAAATCAGTTGCCCAGCGATTATTCAGAAACGCCTTGGAAAGATGAAAGTGTATTTTTAACACTGGAATGAGAACACAGT
AACGCTTTTGAGCTTCAAATTGGATTGCTTTACCGAGCTGAAGAGTTCTCTTTCTTCCTTTAAAAATACTCGCAAAATTTAAAGAAT
TAAAGACTCTGGGGCTAGGAATGTAGCCCAGTTGGGAGTATGCTTGCTACTATGCTGAAGCCATGGGCTCTTAACACAGCACAGCAT
TAGCATAGAGGGGTGTTGCACACTGGCAACGCCAGCAGGAGGAGGATCAAGAGTGCAAAGCTAGCCTTGGCTACATGAGATAGATAC
TGTTTCAAAAACAGCATCAAGAACAACTCAGATACCCCGAAGGGCAGGGAATATGTGCTCAGTACATGGTTTCTTTACCTTTCCTCC
TCCTTTGGAATTTGTAGTTGGAAACCTTTAAGTAGATTTTATATATGTGTGTGTGTGTGTGTGTGTGTGTGTGTGTATAATTTAAAA
CCACACTCTGCTCTGTCTAAATCTCATAAGCAAGCACTAACTACTCCAGCTCATTCCAGTTTGTGTTCTCACACATGGAACTATGGA
GTGCATATAGTATTACAGTTATACTTTGTGCTGTGGAGTGCTTTGTGGCTTCTTCTCATGGATGGGTCTATGCAGGGCAAGCCGTTT
GATAAGAAGGTCTGTGCTGCTTCCTCTCGGTTCCTGGATTGGTGGCAGAGATTTAGTGGCTCTGTCCTCGTGATAAGGCAGGGTAGA
ACAGCATTCTGTGTGTGAGTCTGCTCAGCATCACCAACATTCATCCTTCTAAGCCTTTAGAAAGCTCAAGAACTCATAGAAATAAAC
CCTGTTGTGTTGTTTGGTACCAAGATGAAACATAGCCAAAGCTTGGGGGATTAGGACTGGGAAGGTTCCCAGAAGTAAATCTCAGAC
TTTCTGGATGATATTCCACCATGGCTTTTTGACAAAAGAGCAAGGCCTCAATGTAAGGCTCAATGATGACGTGTGTCTGTGTGTGTG
TGTGTGTGTGTGTACATATCTATCTACCCACTCACCCACCCATCCATCCATCCATCCATCCATCCATCCATCCATCTATCTATCCAT
TCATCTACCTAGTGTTCTGTCTGTCTATCTGTCTGTCTGTCACCTCCCCCTTTTTAATCCAGACTCAATGTGTTTTCACACCTCTCC
AGGTGAGCATCAGGCAGGGACAAGAAGTAGCAGACAGAGTGGCAGCTCAAAATGACCTAAGTAATTGTGGGTGGTCAGGAAAGTAAC
TGGTTTTTAAGTTCCTTCTGGGATTGGGCCACCTCAGAGAGTGCATGGCTGGGTGGGCGTTGTGTGGCTGGCTTTGCCATGCTCTGG
GGGCTGGATCCCACGGCCTGTTGCAGAGCCATTAGTGGGTTCAAGGGGAGGTGCATGGGTGGGGATGAAGTCAGAATTGTGGAGCAG
GTGATGCAAGCACTTCCCTGAAGAGCCATCCCACTGCCTGGCCGCCTACCTGATGACACCACCCAGCGTGGCTTGTGAACGTCACGT
CAGTTCATTGCCGCACAGACACTTTTAATTTGCACAAGCCAGAAGATTTACACTGGGGTTGAGATAGCAGAAGACGAAAAACCCAGT
GGGTTAGTTCAGAGCTCTGACTTTGTTTCTGTTCCTCACCCTGGTGTGAGATGGCTAAGCAACAACAGGGCTAGAGTTCAGATCTAT
TGACGGCATACTTGCCTTGGTACCAGAATTTCTTCCTTTTGAAGTAGTTTAAAGTTTAAATTTTCTTCTTTCTATGTATGGGTGTTT
TGCCTGCGTCTGTGTCTGTGCTGATAGAGATATCAGATCCCCTGAAACCAGAGTTACAGACTCTGGGGCCGGGAATGGAGTAGTACA
TTAAGAATGCCAGTGCTGGGAACTGAGCCTGGGTCCTCTGGAAGAACAGCTAATGCTTTAAAGCACAGAGTTGTCTCTCTGGCCGCT
CCGTGGCTTGAGCTTCTGAAACGGGGTTTTTCCTAGAGGTCCATGGAGAATCTCCTTCAGCCACACCGTGGACCTCTGTGGTACTGA
AGATGCGGCACTGAGGAGCGTCTGCAGCAAGCTGTGTATATTTAGTCCTAGGAGTGAGGATGCTAGACAAAGAGCTAGGTAGAGTGC
AGTGTGGAGGAACTGGAAGGTAGCACGGAACAGTCTTGGTGTAGATGACAACCTGTAGTTTATGCATGGCATCCTGCTGTTACTACC
GTGGTGCAAACGACCCACGAGATGGTATAAAAACCTGTGAGTTCTGAGGTGCAGAAGGAGGAAAGAAACTAAAGAAGCATTAATGGA
GGGAGGAGGCAGAGGCTATGATGAGGCAGGTCTAGCCTGTAAATCCAGTGAACTTCCCACCCTAAACTGACCTGAGTTACCCTGCAA
CAAGCATCCCCACCTCCTGCGATTTCAAGGCACCCATATGTCGCTGTGTGTCGCCTGTCACTTCATCTGGAAGAAGAGCATCCATGT
GGAACATCAGTGGCACCTGTCTTGAAAACTCAGGGCCACTGTTGGTGAGGGGACACCAGTTCTTTGCATAGGGTACCCTCTGGTCAG
ATGGCCTTCCCAATCCGTGCAAGTAGTTTAGCAGGGGAAGGTGTTTTGATTTGCGGCAGGGTAGACAACATGAAGAATGTGGTTTTG
GGATTGGGACACCAAATGGCCACATTCTGGGACTTTTTATCTGGGTAAGGTTGGGCTGGTGTGAGCATTGTGTATCAGGCATTTACA
GAGACCAGGGAAAGGAGAGAGGTATGAGTAGAGCTTTGTCAACAAGAAACCTTGACACAGCCAAAATGGATAAGGAGCTTTTCTCAG
TGTCTCCTGTACCATAAACTCATTCCCTTATCCCACTAGTGTTGAGAATATCAGGTACTCTGTAGGCTAAAGGACACTTGTAGGTCA
GCCTTTAATTATTACATTAATCACAGTATGGAGTAGTACATTAGCTTCTGAAAAGCATGGAGGAATTACTCCAAATCATTTAAAACG
CGTATCGGAAGAAACACATTTGTGAATATAAATAAAAGCATTATTTATAATATCACAAAAACCTGTAATTGATCCAAGCATCCCTCG
CGTGGTGAATGGTGGAATACTACTCAGCCACAGAAAAGGAGTGGAATTCTGAATGTATATAGCAGCATGCATGGGCATGCAGAAGCA
TTGTGTACATCCAATTAGATGATACTCCAGTGCAGACAAAAGTAGTCTGTGCTTATACAAAGCAGATGGATGAATGTCTGGGGCTCA
GCATGTAGGGGTCAACAAGGGAACTTTGTGGCAATCGTGTACTGACCTTGACTGTAGAGATGGTTGAATGGTTGTATACACTTGTCA
ACACACTTAACTCTACATGCAACAGGAGTGGATTTTAAGTGAGTTGTATATCAAAGTATTTTTTTAAAGGCTCATGTTTAAATGTAA
GGCAACTCCTTATAAATTTTGTTATCAATTATAGCTTTTTTTATCGGTAGGAAGGAGTTCAAGTTTCAAAGCATGTAAAAGTGACGT
TTTAGATTATAATCTGTGCAAAACGGGGAACTGTTCATATGCTCAGGCTATCACTCCATTTTAATTCTCTCTAAACAAACTTCTGGA
TTCTATTATTTTCAGAGGGCTTTGGAGACCATTTTAAAGTGCATGTTTTCCCAGGATTTTCCTGTACCTCTTTGGCAAGCTTCATAC
TTACATTGTTTCAAAAGTATAAATAACAGCAGAAGAGAGCTCTGTTTTTGGCATACTCCTTTGTCCTTTCCTGTTGTCCTGATTCTT
TTATGTTAAATCAGCCTGCAGTTCCCGTCCCTTCTGCCTTTGAACTTAGAACATGGGCTCCCGGTCCTTTGCAGGCAGACCCAATGA
AGAGAAAGCATACTGTGCATGGCAGAAACTGGGTAGGACAGAATTCGGGTGCCTGCCTGCCCAGAGCGAGGGTAGTGAAGTGTGGGA
GCAAATTGCTACTTGATAAGCACTTTGGGTTTGGAAATCTGGTGTCACCCACCTGTGTCCCGTGTGGTAGCGCCTATTCTTTATCCT
CCCCCCCCCGCCCCCTCGCTCCTCCTCCTCCAGGTCCTGTGGAGTCAGCTACTTAAACTCATGGTCTAAGACCTCAAAATTTCACAG
TGATCTGTCGTCACAACAAATCTAAAATAATAAAAGGTAATTATGTTTCTGAAGGTACCAGTCTTTTGCATGCTTAGGACTGAGTGT
GCATTAAAGTGGGCTATTGAGTTTTTGAGGGGAGGGGACTGGGGTGTGATAGTGTGTTCGGGTCTAGGGCCAGGCTGTATGTGGTCT
ACCCTACGGTTGGTGGCCTGGACTTGCCCTTTTCCTAGAAAAAGCAGATCAGAAAGAACAGGCAGTTCTGGCCCAGCTTCTTGACGA
TCACCTTTAACCCCCTCAGTGCTGACATCTGGGCTGATGGAGCCCGGGCCCTCTTGGGGATGTGGTGTAGGTCAGGAGGCTTGGGTG
TTCTGCCCATCAGGGCCCTGAGGAACCCTCTGTTTTTGGGGAGATAGCTTATTTTGTGAGCTTGAGGAACTCAGCTCTGAGTTGAGA
ATGCCATTTCACAGGGTGTGATACTTAGCCATGTCAACTTCCATGAGGTTTACGGTCGCCTAGGCGACAAATCTCTGGGTGTGTCTG
TGAGGGGTTTTCTGGGTGAGGTTTACTAAGATGGGAAGACCCACCTAACAGTCAATAAAAAGGAGACCGTGAACTGAGAGGGAGCAA
GTCTCTTTATGCTTCCTGACAGCAGATGCAACCAGGCCAGCTGCCTCGGGCTTCTGCCGCTGTGACTCTCTGATAGAATGGACATCA
CCCAGTATCTGTGAGCCAAAATAAGGGCCCCCTTCCTTAAGTTGCTTTATCAGGTACTTTGTCACAGCCAGAGGGCAAGTAATCACT
GCAGAGGGATGTCTAGAACATTAGATGTGATGAGGGAGTGTGGTAGGCCTTTGTAAACCCTTCTCTGTTGGGAAGCTGGCTTTGGAC
CAGCTGCCGTGGAGCCGAGCCGACTGCCTGGTAACGCCTTGTGATGTCTGGTAACGCCCTGTTCATTTTGTAAATCCAGGGGACGCG
GAGGGACAAGGCCAGAATAATTTGCAGTCACAGGTGCTGCTTTGTTAGCAGCACAGCCTTTTTGAGATGGGGACAGTGTCCCTTCAA
TTTCCTGGTTTGCTCTGCACGTCAGTTTAATACACAGGCATGTGACTAGAATCACAGACCATGGCTGCCAGGCTCCGAGTAAATGCG
CTCACTGGAGGCTCATTACCTGTGATTGCAGACACAGGGAGGAGATTTGACTGTGGAAATGGAGCTTGATGTGCTATTGAAATAACT
TGGCAGGTCGGTAGGAAAGGCGTGGGGGGTGAGTAGAAGGTGGGGGGGGGCTCCCACAGTGATATCCCCTCCACACATCGGTAGCAA
GCAATATTGTCCCATCTGATGAGACATGCACTGTATTTTGTTCTGCTCTACAAATAAGAAAACTCCTATCTATACTTATCACAAAGT
GATGAAATTTTGGGGGATCATGATAAGTATTTTTTAAAAGCTTAATTGTATTATTGGTGTTCCAGCTTCGGACTTATTTCTGGTGCC
CTATAAGTGGCCAGCAGCCTGGTCCCTGGGTGCAGATCTAGGCAGTGGGAGAAAGGAAACAATTATCCAGCAAATGCGAAGAAATCC
TGCCAAGGAGCAAGAAAGACACCCCTCACCCCCTGGCCAATTACAGAAAATTGGTGTTTATAGGGAATTAAGTTGTCTGGTAATGAT
GCTTTCTCTTCCTTTGTCTGGTAGAGCCATCGTCAGAGCCATAAGTGAAAGAAAGCCAGAGTGTTTCCGGCCTCTGTGCTGAGGCGG
TGACCCTGCCTGTAAGCACCCACCTTTGGGGGCTCAGCCGCATTGGGAGCAGGATTGAGCTTACTTCCAGGAGCCTAGTTGACCCTA
AAATACAGTGAAGCGAGTGAGGTGTGTTTATTCCAGCTTGCTGGCTCTGCTTGCTATGCGGGAACTTGGTGTTCTGGTGTACAGCTG
TCTGGTGCTAAAGATGACTTGGGTCTGGGTTGCAGCCCGTGTCGATCCTGGGAAACTCTCTTCCTATGTTGGGAACATTGAGAGGGT
CCTGGGGTGTGACTCTAGTCTCAACAGCGCTGCCTTCTTACATCACTCTACCTGTCCTGGATCTGCTTTCCCACGGGAACAATGTCA
CGGTACTTTCTAGGCACCGTCTACAGTTTGTAATCTATCTTCTCACCCGAAGAATGGTATGGCACGTGACAGACATGAAGACATCTT
TAGAAAACAAAGCTCTCCACGAGTGTAAACTGTGACTGATGACACACTCAGTGACCTTATCTGAGAGCTCATATTAGAGAGACAGTA
TTTGCATCTTTTAGGGGCCCATGTACCAAGGTAAAGCCGTGGCTTCCCTCCTTGAAGCCCTTTAGGGCACATCAAGATGCCTCAGGG
GACTGAATCTAAGCATAACTGGATTTTATTTGCTTTCCACTTCTCCTTTGTTTAATCTGCTCTGTTCTTTATTTCCGAGTACTGGGA
AGGATAGGAAATATGTAAGATTTTTCATATCAGTAAACAGAAATAAATGGGAGGCCCGTGGAAAGAGAGAAGAGACTATATTAAACA
TATTCAAAGCAAGAGGCAGGAGAAGGGAGTCTGTGAAAGTTGGCAGCCGGCAACCCCAAGGTTCGTCTTAAGAAATGACAGGCTTGG
AGAGTGAAGCGCGGGGCTGTAGCATTTTTGGAAAATTGAAGTTAACACCAACGCTGCCCTTTCTTTGCTCGGCGAGACATATGGCAG
GGACAGATTCCTGTGATCCTCTGCAGTCGTGTGACATTGGCGCATTTGGCTCGTTTTTTTTTAAAAAGGCTCAGCAAAGTCTTAGGA
GACAGAACGCCTCTTTACCTCTTTCATCCTGTGGGCCCACGAGGAAGTTAATTCCTTTTTAACTTTTTGAGACATACTGGTTTCACA
AAACAGGAAAATAAAGGGTCTCTCAAAGGACTGAGATAGACTAACCAGTCTGCAGAGTAGGCAGGAGCAGGCTGAGCGACAGGACAA
GACAGGATGGCTGGCAAAGGGTGTGGTGAGGGCATGGCGGTGGTGCTGCATGCCTTGAGGCCTTGCATCGGATCCCCACCGTATGAG
CATAGGATCACCTGCGGGAGGACACTGCAGAGGACTAGAAAGCTTATGGCCCATGTCAGAGCACCGGGGTGATTCTGTACTGTACCT
GTCCTGCGTACTGCAGAGTTCCTGGTCAGGATTCACTCACATGTGAGTATAGCAGTATGTGCTTGCTTTCCTGGAGCCAAGGAAAAG
GCAGACAGCTTAAGAAGAGACTGAGTAATTAGGTTTATTTAGAAGGTGAGAGACAAATTGGCAGTGTGGATATCCAGGAGAACACAA
ATAGGTCTTTGTTTGTACCCCAGACCTTTAGAAATCATGGAGTCTGGGTGCTCCTAGATTATTCTGTAGCCACAGGAGATTTGACCG
GCTTGTGCCAGACAGTCTCTATAATTGAATTCTTGGTGCCCACTGCATACCACCTCCTTCCCAGCATCCTCGGCTCCATTTGGTTTT
GTGTCTGCCATGTCACGACTGGACCACGTTTTCCCTTCTCTGGCTTCTTCACGTTTCTTGGAGGACGGCTTGGGAGCAGCCCCTTGG
CACACAGTGGTGTGCTCTGGGTGCTAGCC)CGGCAGGACATCCGCTACAGGAAGAACTGCTATCCCTCTGCCCCCTCACCCTTGGCT
GGTGGCCGTGCCCCCTTCCAGCTGCCAGGGGGTTCAGTTCCTTGCCTTTGTCTATAGGGTGGGGGAGGGGGGAGCACTACCTCCTTG
GCTCCCCATCTTCATTTGGAAGCCAAAGTCGCTCCTCCGACGGGGCACTGTGTTGCCTAGGCATGCAGGTGGTCCATGGGAAGATTG
GAAAGGGTTGCTGCTACTTGGTGTGTGGTGPACTGGCAGGGGAGTGTGGAGTGCCATTGCTTGTGGCTATGAGGGAGCTGTAGAAAG
AGCATCTATAGTTAGACCCTGTGCTGAAGAGCCCCTTTCCACGTAGTCTGTGTAGACCCTGGAGACTATAGAAGCCGCTTCTCTCTG
GATTAACTGTCGCAGCAGCCTCCTCTGGAGGATCATTGGCAAGTGTTGTAAAAGCCTCAGCTTCCTCCTGGTGTAAGTGAAGAAGAG
CTTATCTGCCTTCTTGCCTGCTTATGAAATAACTAGAGGGTCACCCGTTCATCTACATAGTCAGTGTCCTACACCTATCTCTGCTTT
TTTTTTGAAAAACTTGTTACCTTGGAAACAGTGCCCCAGCTTAGAAATTACATAAGGCTAGCTGTCTGGTTTTTTGTTTTTTTTTTT
TTTTTTTTGTATTTTGTATTTTGTATTTTTGTTTTTCTCCCCACAGGAAAAGCAGAGGAACAGGGGAAAGGTTATAGATTTTTTTTT
TCTAAAATATCTTCAGGGGTTAGGAGAACAGGGTGGGGAAGGAAAGGGAGAGATGTGTTCTTCTGGTAACATTTGTAGAATACTCAG
CTGAAGTTCTTTAAAGATACGATTTGGTTGGTAGCCAGTGAGCTCTAGCGGCCCATCTTCTCAGCGGAGGACTTTGCCTTCACATCT
GGATGGAATGAGGACACTTCCATGGTTCTGAAAGTCCTTTGCTCCACGAGTGAGGAGCCTGGCATCCAGAGGTGACATGAGTCACAC
AAGGGCATTCAGAATCTTCTGTAGAATTCACCATTCCCGACTCAGATGCTGTCAAGGCCGAGGGAGGGAGGTGTCTGAGAATATTAT
TGTCTGGAAGTCAAGTAGCAGTCAGCTTGCTTGCTTGGTGAAGAAACAGAGCAACCTGGCCTCACGTTTTCAGTCCCTTGTTCCTGG
CAATAAAAGGATGCTGTGCACGGAAATTGTCAAAAACATGCCTTCCCATCCAAAGCCTGATCGGTCCTCTTGGTAACTGTTTTGTCT
TTTTGGTTTTAGTTTTGTGAAGTTCCTCTCACTTTGCTTTTCTATAGTGATCTACCTACGACATGAGGAAACACACTGACTTTTATA
TGTTTTCTTCTTAAAAATGTATATGGAACATGTTAGCCCAAGGGAAGTACAGAAACTTAGAAAGAGTTTTGAGGAAAGCGGTAAGTT
TTAAACTCAGCACAGTGTTCCAGTTGTGCCGTTGAGGCAAATGTTTGGTGTAGTAGTGGGGACTGTGTAGAATTTCTTTCTGAATGT
TGTTGTGTATTCATCCTGCATTGGCAAATATATCTTCGTACCCCCCAGCTCCAGCATAGGCAGGCAGTGTATTTATTTATTCGGTGA
TACAGCAGTTGCTTAGCTGACCTTAAGTGCTTGCCGTACACTGTGCTGGGAAGCATAGTTGGATACTGTTCATAACCTCTGCTAAAG
CTTGGACATTCATGTTGAATTGGTTTCCGGACATITTTAGATAAAAAGAGCGGTGTGTGTGTGTGTTTTGGGTGTGGTAGGTGCATG
TGTGGGTGTATTTATGTGTGTATGTGTGTGTATATGTGTGTGGTGTGGGTGTATATGTGTGTGTGGTATGTGTGGTGTATAGGTGAT
GTGTGTGTATATATGTGTGTGTAGTGGGTGTATATGTGTGTGTGGTATGTGTGGTGTATAGGTGATGTGTGTGTATATATGTGTGTG
TATATGTGTGTGGTGTGATGTGTATGTGTGTTGGGTGTGATAGATGTATGTGTGGGAGTATATATGTGTGTGGTGTGGGTGTATATA
TATGTGTGTATGTGTGTGTATGTGTGTGGTGTGGGTGTATATGTGTGTGAGTATATGTGTGTGTGGTGTGGGTGTATATATGTGGTG
TGTGTGTGTGTGTGTGTGTGTATGCAAGTGTGGTGTATGTATTGCTGGAAGTGGCCATGTGTGTGTGAATGCCAGGTGGCTTCCTCC
ATCITTGTTCACCATTTGTGCAGACAAGGTCTCTGATGGCACCTAAGCTCACAGTTAGACTCACCAGCCAGTGAACCCCTGAATCTT
CCTGTCTCCCCCTCCCAGCATGGGGATTATAAGAGCACTGCACCTCGACAACTAGACTTTTTATGTGAATGCTTGGAACCTGAACTT
AGGTCCTCCAGCACTCATGATATTCACTGCACTATCTCACAGCCTCTGAGGGGAACATTTTTGTTGCTTGCATATATTGGGAATGAT
TGGTTTTTTCATACAGTGGTATTAGTCCTACTGTTCTTACAGTAAGAATAATTCCTGACATCTCCCTGCTGTGTCTGAGGGTAGGGT
AGGGATCCGTGAACTTCGGCATAGGGGTAGTTTCTGGACAGTGGGACGAGGAGCTGGGCAGAGTGGAGCCGAGTCATGGAATGTGCG
AGCCAGTACCTCGTGCTGGTACTGGCTGCACAGTAGAGAGGGTTGGAGGAGCAGTGACCTGGGAGTAGAAGATTCTGCAGATGCGTG
GCTGGAACTTCCTAGCAGGTATTCAGTGTACAGTTTTGCCATTATTCCCTAGGTCCCATGTGAGAGTGATCTAGTGGTGGTCTCTCT
CGTCACCAGTGGAGATGAGCTCCTGGACACTCCTATAACCCAGCTATGAGCTAGTATCTCACCCTGGTCTCCAGGCTGCAGGAAAGC
CTGTTGGCCAGCTCTTGCCTCTACCCACCATGACCTCTCTCTCTCTCTAAGGCAAATCACTGGCTCCACAGAGTATTTAGAAATATT
TCTATTTTTTTTAAAGGTTTTTAAGAAGGTTGAACTGAAGACAAAGGAATGGTCTAATGAACAGCAAAATATAATGACTCAGTTATA
AAATGTCAAGATGATAATACAATAAATAAAAAGTTAATCCATTTTGGACTCCACTGTGTTGCTCTTGTTTGTGGAGAACTGTCATTG
TGGCAGAGTAATTAAAACCCTACAGAGGGTTAGGAGTCAAGAAGGAGAGAAAAGAGGCAAGGGGGAGCTGCCTAGAAGACAGGAGAA
CTTTCTAGAGTGCTGAGGCATGCAGTTCCGAGAGGTGATATTTTGCTTCTCTGACTGCACATTTCACATCTATGAGGGCTAAGCACC
TGGGTTACACAGGTGACCCGAATTGGAAGTGATCACGATCTGAGATGGACCTGCGCTTGCTCTGTAAATCGCATGGTTGCCAACTAA
GCCACGAGCCCTGCCTCACACTGCTTTACTGCATTTTCAAAAGACTTTCTTATTTCTAGACCTTACTAGGTCTTGCTCACTGTTTGT
GCTAACACACGCCAAAGAAGATGAAAGCCAGAGCGGTATGTTACCCTCTGCCGTTGAGCTCCTGTTTGTGTCGTAGCCTGGTCAGTC
TTCAAGTGCCAACAAGTCGTTCTGGTTCATTTTTCTTGCCCTTCTTTATTGGCTCTGAGGTTTTCAGTGGTCAGCCTGTGCTTATAG
CTTCAAGTCATTAACTTTGATCAAAACAGGAGGGATTTGAGAAGTCAAAGGACAGAGGAAAAAATCCAGAGCTAGCCAGAATCTTTG
GTTTTCTATAGAAACGAGTGAGAGAGAGGGAACGTTCCAGGAATCAATATACTGGCTCTGCCCTTCCCACCCTGGCCTAGTTAAAGT
GGCTTCTTCCTATACCATTGCACGTCTGGCTGGCATTGGGAATATTCTTAGGTTGTCAGGTTCCATTCTGGAATTAAAAATGTATAA
TTATCTTTTTAAACATTTTTGTGTATTAGCTTTAAAATCAAATAACATAAATGAATCAGATGTACTTTTTGACTGTCTTTTGGTTTC
TTGCTGCCAAGGACACCCCACCGTGGCATTTGAGTCCAGCTGGCAGCGATACTAGACTCAGCACAGGTCACCTTTGGGTTGCTCTGC
TATTTCAGTTCTGTTGGAAGTGTTGTGCACGGGGGGCCCTTGCCTGCCTGGGACGTGTGAGCCTGAGGTGAGTTTGCCCACACAGCC
TTTCTCTGGTTTGCAAGAGAGTCACAGGGGTCAGCTGGCATCTGCAGTCTAGCTGAGGTGATTGTCTATGTGAATGTTTTCTCAGAA
CCAGATAAACCTGTGTCACTTGCAACACTACCACTACACACATCGGCACTAGCTTGGTGACAGTGGATGGTTCTACTCTAAAATGGT
TTTTCAGAGACTCGACTGTGTGAAGCCTACATAGAAATGAGTATGATTGTAATTATGATGAGTAAGAGCTTATTTTCTTTTAAAGAA
GGTAATTGAAAATTTAATTTCAACTGACATGTCTAATCTCTTCTATAAACAGATAGAGTTTTATTTTGAGAAAGGAGCCTGGATAGT
CCAGGGTGTCCTTGGCCTCGTTAATGAGACAGAGATGACTGTGACATGTTTGTTAGCCCTTGTGTGAGTGGTGATATGACTGGAGAG
CACTTCCACATGCAGCTTCTATGGCATTGGACAGGTACTGGGGAAGCACTCTACCAACCGAGCTATAGTCCTGGCCCTTTAAAAGTC
TGCAGGAGTTTGTTACATGGCCCAAGCTGGCCTAGAACACACTATTCTGCCTCTGCCTCTCAAATGCTGATATTACAGCTATGCTTG
CTACCGTGGCTAGAAATACGAAAGTGGCCGGGCGTGGTGGCGCACGCCTTTAATCCCAGCACTTAGGAGGCAGAGGCAGGCGGATTT
CTGAGTTCGAGGCCAGCCTGGTCTACAAAGTGAGTTCCAGGACAGCCAGGGCTATACAGAGAAACCCTGTCTTAAAAAAAACAAAAC
AACAACAAAAGAAATACGAAGAAAGTGTATTCATTCCGTAGACAAATCACTAATAAAAGAATCTCACAGTGTCCTACCTAGCCAACT
GTCACATACTGTGGATACTCAGATCTTCGTAAAGGTGGGTGGGAACTTTCATTCATTTTGATTGGGGTTTGTCGAGTAACACGGACC
TGCAGAGGATGTCGTCATTGAGTAACACGGACCTGCAGAGAGTATTGTCATCATTTTCTGATTTGTAGATGGCCGCCCTTTACACTG
CTGATGAAGCTCAGGGTTGACTAAACACACCTCCGCAGCCTTTGCCTTCTACATAGGCCTGAACTCGAGAGAAAGCTGGGCGTAGAG
CAGCCCCTGAGAAGCCTAATAGGGAGAGAGCTGGGCGTGAAAGCCGCCATGCTGAACTACCCCACATAACACAGTCTTCTTGGCTGC
CTTGTTTTCTTAATGGTTTTAGTGGGGAAACCAATAACATGTTCGTTTGAAGAAGCAGTTAAAAAATAATCAAACTTAACTGAAAAC
GGAGCCCCCGCCTTTGTTAAACATCCGTTTATTTGGCTTGCTTTCTGTCACGTCTCTCTGAAAACAGAGCCATGAGTCCTTTCTGTT
TAACTGATCTGCTCGGTGCTAACCCAGTGCCCTTCATAGCTCAAGTCTAGGGTCTGAGGTCTCTGTGAAAGTGCTGTGCCTACACCA
GGTTTGCCATGGAGGCCGGGAACTGGTTTTCGTGTGCAGTGACGTTGTGTTGGTCTTGAAGCACGGGAAGAGGGCTTGAGGATGGTG
GAAGAGGTCAGAGCGAAGTAAGGGAATCGCATAGCCAGCTTCGGGCCTGGAGCAGGGGCAGGGCGTGGATGCTGGCGATTGAGAGTT
GGGCATGGGGGCGGGGTGCAGGTGCCAGTGTAGAGGAGTCTCTCCTCCCTGCCCAAGCCTCATTGGTCTTTATTTCCTTGCCATCAA
TAATGACATCAGGGCTGGAACAGAGGATACATTCAGTGTTTCGGATGCACCCACAAGTGAGAAGCTGCCATTGCCACCTGCCTGAAG
CTTCCAGCCGGCATATGGAGGGTCCCAGCCGGCATATGGAGGGTCCGTGCACTTCTTCAGGAAGCATCAGACCTTTGGCTCAGCAGA
AGGGATTCCTGAGCAGGTGTCTGGGTGGGAGGTGAGGCAGGAAGGCAGGCTGATGTTACTACCTAACGGAGAACTGCGGCGTCTTTT
TCCCTTTCTTCTGTAGTCAAGTGGCTCCTAGTTCAAGGGCAAAAGTTGGGAACAGTGTTCAGCTATGCGGGCCGGCCATACTCTGTT
CTTCCTTGGAAGAGACAGCCGCAGATGGCCCATCCCTTTGCTGTAGCTCACCAGTCTTATCTGCTCAGCATTAGGCTCAGGGAACTT
TCCAAGCACAAAGGAACCATTCTCGGGTTCCAAGACTGGAAAGGCCTTGGAACTGCGTTTTTAAAATTTCACTTTATTTATTATTTA
TTGTTTTAAACATCTTATCCCTGAAGCCCAGCACAGGGGCTGGCAGACTATAAATGCTAATAGAAAAAGATAAAAGAAAAAAAAAAA
GGCAGTTCAGGCCTGGTTTCTTGGAACAGAGCTGAGAGGAGAATTCGCCTTAGACTAGATGAGCCGAGAGTCACTTTAGGATTATTT
CCTCTTATCGAAGTGTAAGTATTTTGGGGGTGGGATGTGAGTGAACTCCCTCACGGCTGTCGGAGATAAAGTGTGGGGGTTTCTGCT
CACGTTGCAAGTTCCCGTCTCAGGAGGAGGTTTTGTGAGACTTGGTCTGCCTCCTGACCGTACAACTTTGCATAATTGATTAACCAA
GTATCGCTGTTTGCATTCCCTTCCTAAAATAAATTCCACTTTTTAATTAACTCTCCAGAATTAATAAGTGCCAAAAACGCAGGATGT
GTGTTGAATCTTGTGATTCGATGTGATGAAGGCAGCTCCCAGGGAAAACAAGTCTAACAAATGATTTTGAGTCCGTGAAGGTATTCT
TAGTTCCTGAGCATGGTAAACAGCTCTCTAAAAGCAGTTAATGCCCGCTGGCCTTCGTGTGGGCCAAGCGCCATGCTATGCCTAGGG
CATCTCGGCTACTCCCTTTGGTTAGCTCTTTATGAACCCAGTACCTAGCTGAGTGGCAGAGCCTTTGCCTATAACCGAGGGATGGGT
TGGGATAGGGAATTGGGTTCCATCCGCAGAACCTCAGGAACACACCTGCCACATGAACTGGGTGTGGCGGCGCACACCTGTGAACCC
AGTTCGTGGGAGCGGAAGCAGGAAGATTGAAAGCAGAGGTTCAAGGCTATCCTCAGCCAGCCTGGATTATACAACTCTAAGATTAGC
TTTCTGATGATCCGTATCTCCACAGAGAAGCCGTTTGCCAAATTGACGCTGCCAGTGTCTAGCAGCCCTGAATTTGATCTGCAAGGC
CAGACTCTCAAAACAGTGTTCCTCCAAATCAGGCCACTCCCCTCCGCTACAACCCCCTATTAGCCCCTTTGTATCTTCAGGTTGTCT
TTACTGCAGCATCTTCTCTCAGGTCCACTGTGGACTGTCTGGTCAGACGTGCTAAACATGGTGAGTTTGTTGGTATTGGAAATGTCC
CAAATACATGGCTCCTAATATAGAACCCAACTTAAGGAAGTCTTTAGGCAAATGACACCACTTGTATAATGGTAGGTCACAGCCCTC
TAATGACTTGGAGTCAATGCTGGGTCATTCTGTGCCTGTGTGTTTCACAGAAGCAGAAACCTAGAGCCGCAAAACCAAGCAGGGAGG
TTACTGGAGCCTCACGGGGAGAACACAGCTAAGGATTCTGGTGGGCAGGCTCTTGGGAAACAGGAAGAGCGGAAAACATTGCAAATG
AGTAGCACCCCCATAGCTTCCAAAGGAGAGGTGAAGTCTTCACCGGAGTCCAAAACTGGCGATCCGGCGGCTGTCCTCTGAATGAGC
AGAAATCCGAAAAAGGCAGCCGGAGTATGGTTTTTCAAATTGGACTTTAATTTTTTACTTTCAAATAGACTTTAAAGCAGTGGTTTT
CTATAGCTTTATGAATGAGGAGGATCCAGTGAAGTGTCGTATGTGAATAAACAGGTGTTAGACATGACTGTGTGGCCATCATGGATG
GCCTACTGTGATTAATGCACGCCTAGGTTATTTTCATTAAAAAGTAAAATGTGTTCGTTCCTATTGTAACAAGAAAGGCTAAGTATT
AGGGGCAGAGGAAGGACGGAGTGCAGGTAACCGACGACGTGAGTTATAAAAGAGGATTCAAAGCTAATTGCTTTGCTAGTCCTAACC
CTGTGGTAGATTCTCTTCTTTTCAGTGGTAAGTGTATGACATGGATTTGATGCTGAAGTCTTAATAACACTGGAAAAGTCGATCGTG
CCTATAAGTCTTGAGACAAAGAAGAAAGCAATACTTTCTGATGTTTTTTTAAATCCTCTTGTAAATGATGTGAGCATAAAGAACGTG
ATAAAACACTGCAGCCCTTCTGAGACGGAGAGGAAATACAATAAGCTGATACTTGAATGTAGCACTGAGTATCAGAGAGTTGTAGTT
TCTGTGGAAACAGAAAAAGTTTTCAGAATATCGTGTAGCTGTGTAATATGTTGATTATTTAAGGGAGGGGGAGCCTCGAAGGAGATT
TTTATTTAGCTTTAATGGTTTTCTTTATTTTCCTTTCGTTTTGTTTTTGTTTTGTTTTTATGAGAGATTGTTATTGTTGTGAATTAA
GAAAGAAAACTGGTGAGATATTATTCCTGATCCTCTGGACCAATCTAAGGAAACAAAGGAATTACTGCCCCACCCCCACCCCCTTGT
GGTCCTGACTGGCCAGGACAGACCTTAGCATGAGAAAACAAGTAGGAGAACTAGAGACCTGGTCCCCAAGGCTCTCACACTGCCCCC
ACCTGCATCTGTCGAGGGCTCAGGACTGAGGGATTAGTGGTGATAGTGTCTCCTTGGACGGACTGACAGTGACGAGCTGTTTCTTCT
CTAAAAATGAACTCCCCAGCCAGCTGTGGATATACACTCAAGTTTTTAGTGACATTTTAGAAAATCTGCATATATGTCCAAGAAAGG
CCTGTGAAATGTTTGAGAGTTTAAGATTATTCTGAAGCTTTATAAAAGTGTTTTCTGTGATTTTTGTTTTCTGGATATTTGTATCCC
TGCCTGTGTGCTATCATGGTTAATTTGCACTGTATCACTATGCCTGAGAAGCCTCTATTAGTGACTGTCCCTGTCATCCTGTGAGCT
GTGTCTGTCACCGCTCTGTGGACAGGAGTGTTCCCAGGAGACCCGGTCTGAAATGCTTGGACTTAGGAAGGCCCCTGGGAGAGAGTT
CAAGATCTATGAAGGATGACTATTTAAGACTGGCCAGGAATGAGCCCAACCCATTCACTGCACAGAGAGACCATTGCTGCTGGTGCA
AAGAGGGGAATCCCCTGTAGCAGAACTCAGCTCCGGAAGGTGGAGTCTGCCCATTGTTAACTGTTGATACCTCAGGATGGCCGTGGT
AGACGTCAGTTCAACTTCAGTGAGGAGGCAGAAATACAGGCTACACCTTTAAAGGTTTGAGAGGTTGGCTGTTCAGGGTGACAAGGA
TTACCTTAGGGATTAATGAATTAAGAGAGGTGGAAAGCTAAAGCCGTCCTTTCTGCACCATTCAGTTCCATTGGACGCATACTTTCC
CAGGCTGAGTGGACCATTTGCCTTGGCGGAAACTCACGGCCTTTGAAAATGATGACATCGGGCCAGTTCTGTAACTATGATTTATGC
TCGAGTCTGTACACCGTGGGTCAGATTAATTCAGTGCTGCCCTAAGGGACCCAGTTGTTGAAGAACTGCTATCTCCACACCCAGAAG
CTGTGTTAATGGTCCACTGATGCCCCGAGAGCCCGTGTTCTTAAGGTACCAAAGTGCTTTTGTGTTTGCTGCAGGATTATTTTAAGT
CACAGGCGCACTTTGGAGATGATCACCACTCACATTTTGCTTAAAAGAGTAAGACAAGCCAGCCTGAGGTCCAGAATGGTCGGTACT
GATGCTGCACCTTGGTCGTTGGTGGGAGAGATGAGATCAGTATTTAACAGTTGGAGAATTCTCAGTCTTGTCAATCAAAGCATATTA
CATGGGTGTTTCCACACCATTTGAAGTGATGATTTAAATTCATTTCTAGTTGTGAGCTGTGCGAGAGTGGACGCCCTGGGCTTTGGA
AGCTGCTCTTTCTCCACGTGGCCTTTTAAGCTGCGGTAGATGTTGGTGAGGAAAGGCGTTCCTGTGATGTTTTGTCTTTTCTTTTTT
TCAACAAGTGGTTCCCCCCACCCCTGCTCCCACGAGAGGCAGGGTTTCTCTGTGTAGCCCTGGCTGTCCTGGAACTCCCTCTGTAGA
CCAGGCTGGCCTCGAACTCAGAAATCCACCTGCCTCTGCCTCCCAAGTGCTGGGATTAAAGGCGTGCGCCACCGCTGCCTTTCGAGA
TGTATTTTCTTAAGGGTGTTTTTCCTTGTACTTCCAGGTCAGACCTTACGAGGACAAACTCAATTGTGGATTTAATGTCTGTGTACT
CCTCAGCGTCTCCAGGTCCCCTTCAGAACTTTGTAGAAATTGTTAGTGTTGTTTGGGTTGATAGCCATGGCCAACCCAGGGACAGAA
CAAAGGAGACATCTCTAATGAAAGGAAAGAGGGTAGAGGCGCATTGGTGTGTATCCTCCGAGACTCCTTCCCTCACAGGAAAGAATG
TGTATCCTTTCCTCTGGGGGAAGGGTGGGTGACTGGATCAGCCCAGACAAAAACAACATATGGTTAGTTTTCGGAGGACTTGTAGAC
AGATGGCAACCTATAGGCTAATTATGCAGTGGGCGACATTCTTTAAACAAATCACTGCTTTTGTGGCATGGTTCTTACTCACAAGGT
GACAATCTGAACTGTTTATTGTCACACCAGGTAAACATGTGCCAGGTGAATGAGGGAAGGTGCTACCCGAGGGAACATAGTTATATT
GAGTTTCCATTTGCATCTTGATCTTCCAGGCCAAAAGAACAAAGAACGGCAGGAAATTCACAGAATAAGACAAACCTTCAGGTCAGG
GTAGGGTGTAGCCCAGTGGCACACTGGAGGGCTAGTGTGTGTAAGGCTCTGGATCCAGCCCCCTAGCAGCTAACAGATCACCAAAGT
CGTCATGCTAGGGGCTGTTGCCTTGGGGGAGATGACCTGTGTCAACCTTCTTGTACTCACTCGGAATGCGATGTTTCCCGTGTCTCT
GCCATCAGAAAGCAGGTTGTCAGAGCACTGTGTCAGGCTTTGGATGAGCAAGGTGGGCAGGTCACCCCTCAGAGTTCTGCCCTGGCT
CCTTCCTGTCCAATCCTGTGTCAGCATGTTCAAGGTCGCCCTCACCCACTCCTCCTTCATGCTTCCTCTTTCTCATTTAGATTTTTC
TTCACTGGCCTTTTAACTATCTGCAGTACAGGTTTTGTTTTAAGTTTCATTATTGAGTGTCTCCTTCCACGAGGGTTGCTTCACAAG
GACGAGGGGCCCAGTCTGTTCTCTGCCTGTGACAGGGCTTTGTGTTTGCAGAGGTAAGGGAGGGGTCCATTAAAACTGCAACCCAAC
CTTTTTCCTCCCCAGTTTGTAGGCATGCCACGTGGGCCCGGTGTGCATTGGTCATGGGCTCTGATGGCTGGTCCCACCCTGTCCTTG
AGAACAGATTAGGGGCTCATTGTAATGTTTGCAATGTCTCCAGTCCCACTCTCTGATGTAGACTTGTTTTTGAACTGAATGAGCCAA
CTGACTCAGCTGAGGAGATGGTCAGGAGCCAGGCTTGTTCCTGGCCAGCAGCCTGAATGGTGGGTGGTTTCCTGTCCATCCTGTTAC
ATAGGTTCAACCTAAGGGGTGTGGCCATGGTTTAAATTTAGGTCCGATTGGTGTTCAGCTTGGCTCTACCAGGAGTGCTTTACACAC
AGGATTCCTGTTTCAGTGTTAACTTTCTTACATCTCACAAAGCCACAGTACATGTATGTTTTTACTTATATGATTGAGAAACTATGA
AAAGGTGCTACTAATAGCTTAATAGTGCTTTTAGAATAGTTTTACTAATATTATAAATTTGAGAAATAGCCCAGCACAGTGGGTTGG
AGTCCTTCTCTGGAGTCGTATTTGCCAGCCCTACAAGGGCTCTGATGGCCCATGGCCACCTCTAAGACACTGTGACCTCCGTCTCTG
GAAAGCTCCCCTCTTCATGCAATATATGCTATCTCATCTCTCCCCTGGTTAGCTGGCTAACTTAGATTTTGTTGGGACTGTCTGGCC
AGATTGCTTTTTATACTTAAGTTTGTTGCTTTGTAAAGAGCCCCTGGTTTGAAAGTAGTCATCAGACTAGAATCTGTTAGAATTAGG
GATGTGGGCAGATGCATTCTGACCGAGCTGCAGGAACTTTCTATATGCTGGTTGGTTGGTTTAGATGGTTTGGTTCCAGGCCTTGAG
AGGCCAGGTCCTTGGTCTTCTCAAGGATTCATTAGGTGAGGAAGTGCATGTATCCCTCACAGGGAAGTTGGGCTCAGTTCTTAGGAG
ATGGGGTATGACTGTGACAAGGTGGGAGGTCTATACAAGAGCAAAGGTGCCCTGGTGAGAGGTCTTAAACCTGATTGAGGGCACAGG
TAGTAGCACTGAGGACTCACAGAAAGATTAGTTCCTTGGTGCTGTTCCCTGTATCCTCATCTCTGATTGGGTATTTTGTTGTTGTTG
GTTCATTGTATTTAGTAGATTGGGTTAATTCAAAAAGGGAAGGTAGAAGAGCAAAAAGACTTTTTTTTGTATTCATTTTTAAAAAGG
AATACACAGTCAAGTTCTTACTACAAAATAAATGATCCTTTTGAGGGGAGTCAGAGGGCCTTGTAAAGTGGATACTTTGTAATATAT
ACAGAATAAAGTGGATACTTTGTAATATATTTATATATTATATATGGAATAAAGTAGATACTTTGTAATATATATGGAATAATGTGG
ATGCTTTGTAATATATTTATATATTATATGTGGAATAAAGTGGATACTTTGTAATACATATGGAATAAAGTAGATGCTTTGTAATAT
ATATAGAATACAATAAAGTAAGAACCATCTTTAGCCAAATTCCCATAGTAACTATAGTAAGAAAATGTTTCAGGCAGACCCATGGTA
ACCCTCAGTATGGACCACTGAAGTGTGTGCTCAACAAGCTGCACACCAGAGGCCAAGGGTCTGAGGAGCATAGTTGAAATATTTTCT
CCCAATCTTCAAGTCTTCTGCGGAGACAGCAGGGAAATTATACTGAGATAAGTGGTGAATAAGGGTGAGAGAGAAAGATGTAGGCTA
GACATAGCTCTCCAATCAAGACTGTGTTTCTACTTCTTGCTCTTTGTCTGCATTTTTAAAATACCCACCATGCAGTGCTCTCAGCTC
TATTTTCCCTACTGTTTTTCCTACCCAGTGATTGAGGGGAGATTATGCTCTATCAAGAATTCACAGTTTTGGCAGTCTCATGTCGTC
TCTGTCATCTGTTCACTTGACCTCATTCTTGCAAAGGGCATTTGGGTGTGTCCTGGAATTGTCTTAAACTTTCAGTGAATAGAGAAC
TCAGATCACATGGATTGCAGTTTGGAAATCTGCTGTCAGCTGATGAAGCTGAATGAATGTGTGGTCTCGGTGTGGGCACGGCCCTAG
GGTGTGAGTTGTGGCCGAGTATTAGCTGTGTGCCTGGCATGTGCTGTCACAGGTCTGACATGTGCCTCTGTTTCATTTCTGGGAGCT
TCTCCACGTGCTGCCTTCACTGAGCAAATGAGATTAGATTTTTCTAGATCTCTTGGCATGTCTTTGTCACCACCTTTATGACTAAAG
GACATAAAGTTAAGATATGATTGATGTCTAATTGAAAAATAGCCACTTCTGTGGATTGAAACCTTCAAAGAAGCGGACTGGAGAGAT
GGCTCAGAGGTTAGGAATGCTCATTGTTCTTGCAGAGGACTGGGGTTTGATCTCCAGAACACCTACATGGTAGCTCACACTACTCTT
ACCTCCAGTTTCAGAGAATTCAAAACCCTCTTCTGACATCTGCATCTGGTACAGGTACCCTCTAGAATGCATTGATATAATCAGCCA
GTCAATCAATAGCCAGTCTAATCATAGCCAGTCAATCATCAATCAGTCAGTAGTCTCGGTGCTCACTGTTCAATCATTAGGACTCAA
GTTGGATTCCTCAGGAACCCAGGTAGCATGCCTCCTTAATCCCATCATTCCTAGAGAGACGGGGGGGGGGGGGCATGGACAGAATCC
CCAGAAGTACTCAGGCTCGCTCACATGGCATACACTAGACAAGAGACCCTGTCTCAAATGAGACCAAAGGTAAGGACTAACTCTGGA
GGTTGTCCTCTGGCCTCCCCACATGCTCGGTGACATGTGCACACCAGTATTCATGCACATAAACATGTACGCACATAGATACATGAC
AGGTGTCTCACATACACCTCAAAAAGATGAAAAGAAAAAAAAAGTCATTGGCATGAAGGAAAGGCGTTGCTGAATGACAGAATGCCC
TGTTTTCCACCAGCCGTTGGAGTAGACAGCTACCGAGGGTAGCCTCTCAAACAGACTCTTTGCAAATCAACTCCAAGTTCAGCAAAG
CTTCCCCGGTGGCATAGACAGGAACTTGCTCTGAGTTTCGGAGACCCTGAATCTCTCAGGGTCATTTTTGTATCTCTTACCCCCTGT
GATGGTTAATATTGATGGTCAAAGTGCTAGGATCTGGTGGCACATCTACAAGGAATTTTTTTTTTTTAAGATTAGGTTTGAGGTGGG
GAGAATGACCATAAATGTGGACAGAACTGTTTTATGGGCCAGGGTCCTAGACTGAATCAAAAGAAGAAAAGCAACTGAGCAGCAGTG
TCTTCCTGCTCTGTTTCCTGGAGGTGTATGCCCCACGACCAGCTGCCTCACTTCCTCCTACTGCCCTATCATCCTCGCCATGAGGGA
TGGTGCCCTCTATGAGCCAAAAAAATCCAGGTATTTTTTCACAGCAACAAGAAATGAAGTGAATACACTTCCCTAAGGCTTCTCTTG
TGTTTTCCTCTTTTTCCTCTATTCCTACCATTACTGCTTGTCCTTGGTACCTTGTGCCCCTCAGCCGTCTAAGCTCTACTCTGAACA
TAAAAAGGGCTGATTTCTTCATCCTGATAACCCCCTCAGGACATGGCTGCATAAAACCAGATCCTAGCCTGACTCAGGCCAGGCTCA
TGCTGTCTCTAGTTCCCTTCTGTGGCCTGGGAGTCTTGGTGCAGGAATGACAAATCAGTACACAGTCTCCTACTGTTTGCCAGGTCC
CTGGAGGAGCCAGATCAAAGTCAGATGCTCTGTTGGCCCCTGAGAATAAAGATAGAGAAGTCCAGGGTCAGTAGGCATCCAGCTTAA
GTCCACAGTGTCTCCTTCATGACCAGGAGGCCTCCACTGTGGCACTGTGCAAGTTGGCTTTGGCTGCTGTCAGTGTCCACTGTGGAT
CCTGATGTTGCCCATGACCCTTTCTCTAGGAATGCTAGTCTGAGCGTAAGGCCATTGTTCTTCTCTGTCTCTCTTTGCTTAAGAAAG
GCCTCCGGGCAGTGGTGGCGCACGCCTTTAATCCCAGCACATGGGAGGCAGAGGCAGGTGAATTTCTGAGTTCCAGGCCAGCCTGGT
CTACAGAGTTAGTTCCAGGACAGCCAGGGCTACACAGAGAAACCCTGTCTCGAAACAAACAAACAAACAAACAAACAAAAAACCAAA
AACAAAAAAAAAAAAGAAAGAAAGAAAGAAAGAAAGGCTAAGCCCCTTTTTTACCCTTAGACTTAGAACTTTTAACTTGATAGGTGT
GTTGAGAGGGGTTGTTGCACATCTGGTTCTCAGAGTTCATCTCTGAGAGCAGAAGTCTTTTTTTTTTTTTTTCTCATTTCAAAACCC
CACTGTGCCCTGTTCCCCTATCTTATTTCCTTAAAACCTGTTAAGGCCATAATTTTAGAGATGGCATTCCATTTTTCTTTGCCTGAT
TTTCTTGGTCAGCCTTTCAGAGTCAGCCAGCCTTTTTTTTTTTTCTTCCCTCTACTCTTCTCCATTTTAGAAAGGGGCCTGGTCCCA
TCACAATCTACCATCTGTCTCTGATGAGGTGTCCTGAGCTCTAAAAACCCATCTGGACACTTTCCACCGTCCTTAAAGGTACAGGAT
GCTCCCGGGTCACAGCTTTATCCTGAAGTCTGAGCACTTTGTACGCGCTTCATTTAGTCGTCTTAGAGAGGGACATTGAAATCTGAG
GCTCCTTGGATTTTCAGGTTAGGGTTATTCAGTCCATAAAGTCTATGCAAGAATTCTGAAATCAAAAGAATTTCCGAGTTCAAGCTA
CCACTGCTCTCAACTGCTTCATATGCAACGCTGTGTTGTTAGCCCTGCTTTACAGGTGAGGATGGTGAAGCCCAGAATCTCAGTCTG
GTTAGAACCGCCTGGGTACCCTTTTCCTCTGCTCTGAATGCCCAGTGCCCTCTAGTGCACCCAGCTACACTCCTGGTTGACCTGCCT
GTCCTCTTGGTGTGAGCCCAGCCCGTTTCTGCATCTCTGTCTCTCTGCCCCAGGCGGTACTTTCCTTCACGCCCTCTCCCTGCCTGT
CATCTCTGAACTCCCGAGGTACTCAAGGATATTCTAGAAGCAGACCCATCTGTTGTGACCCAAGAGCCATGCTGTGTTGCTTGGCAC
AAGTTGGCTAAACTCTTTGGCTCCCTGCAAAACAGGCTTCATAAGGTTCCTAGAGGATTGTATAAAAAAATTCTTTGTAAACAGATC
CCCCTCCCCTGCCATTCTCAACACATCAGCTATTATTCAGACTTCTTACTACGTCTTACAGCAGCAGGATGGATATATAGGAAGGTG
TTTCTACCAGCATGGATGCTCTGAGAAATGAATAGTTACGTGAGTTCACTGTTCAAACACAGTGCACTTACGTAAACCAAGATGGCC
CCATTGCCACTAGACGGTGGGCCCCTCAGACTACTTTTGTATATTTGGCCAGCTGACAGAAAGATTATTATATAATACATGACTCCT
CTTTAATATTGTGACTCACAGGCTGTGAGCACGCCCGGTTTTGTCAGGACATTTTCTTCTAGTTTGTGTGTGTGTGTATGTGTGTGT
TTGCTATAAACAATGCTGGTTTGTGTTGTTTAATTGATGGATGACTAGCCACTGTCAGAGGCCACCCACAGTGACCTGAATGGCACT
AAGCCCCTTGANNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNGGCAAACTCACCTCAGGCTCACACGTCCCAGGCAGGCAAGGGCCC
CCCGTGCACAACACACACACACACACACACACACACACACACACGCACGCTCACACGCACACGTGCACACTCATATGAAGCTAGAAT
TTGCATATGGGAACATGTTTTTGTCTTTCTGAGCCTGGGTTACAGTATTTAATATAGTAATTTCCAGTTCCATCTATTTTCTCTTTC
ACATCCACAATTTCTTCTTCCCGGTGATTGCATAAATTCCATTGTTTCTACGCTCCATGTTGTCATTTTCCACTCTTCAGTGGATGG
ACACCCGGGCAGTGGTGGCTCCTCTTCATTCTCCTGCACCCTCATGGCCCTAGCCAGTGATGGAGACTCAGCTGGTTCTCCTTGTTG
GAACTTTGGTCTGGGAGTGAAGAGACTCAGAGGACAGATTTCTGCTATACATAAATCATTTGCCAGTCACTCATTTTTCAAGGGCTT
GTCACTGTCAGGGAGCTCCTGGGGATAACACTGTTTCTGCCCCAGCCAGGAGCCCACAGGTTCCCTGCACTTGGTACCAGCTGCACA
CAGCTGTCTCTACAGCAGCCCTCTGCAGAGTTTGGCCTCTGCTGGAAGCCAAGGCATGCTTATTGGTCTTCAGGGAATATATTCCTC
AGGCTTGCGTCTGCCATCCAGTGTGCTCTGTGCCTGACACAGGTCATACCTCCCTCCGGTCCCTGATACCACAGCTTCTTAGTCTGA
GCTGCCCTGCCCCTGCTGGCCTGGTAGTAACAGAATCCTAGGCTTTGAGAGGAAGGTGCAGTGCACACATGCTGCCTGTCTGCCTCC
CAGCAGCTGGGCTTCCCCTGTAAAAGCCTCTAACATCCTCAGCTCCCAGGAGGAAACAAGCCTGCTGCTTGTGACACCAGAGCACCA
ATTTGTCACCCTCTTCACTGGCTCTGGCCCTCACCTCCTCCTCTCCTCTATGGCATCTTTCTTTCCAGGGCCTGCTGCAGCTGGGCT
CCAGCCCGACACTGAAGCCCCACGTGGCTGTAGCCTCTGCTGTGGTCTGTCCATGGGGTTGTGGCTACTGCCTTATTCCTAAGCCAA
GGTCTGTCCGTTGAGCATGTCGTTCTCCTGGGAAGCTTGCTTGGGCTGATCCTCGCCCCTCCTTTACCACTGTGCCCAGCCACTTGA
GAAACATGCAGGGCCCACTCAGCACAGAATGCAGTTAGTACCTGTAGCTCTCGCTCCGCTGCAGTTCTGTGAGCCGCCTGCCTCCGC
TGACCCTAGAAAGAGTTCCGCATTGCAAGTGTAGATTTCAGGAACCAGAGTGTGGCTGATGCTTCTCTTAATTTTTTTTTTTTAATT
ACATTTAGCATGTATGGCAAGGTTGTAAAGGTCAGAGCACAACTTGCATGAGAGCATGTCCCCTGTTATCATATATGGGTACTGGTC
AGGCTGTCAGTGAGTGTTTTTGTCTTTCCATCTCCCACCCTCCAGCCAGCTCCCTGAACTGTGAGGCTTTCTCTCCATTTCCCCAGA
GCAGACCCCAGCCCCCCCACTCTCCATCTTTTCCTTTGACCCAGGCCCTGCCCTGTCTTTCTCATTGAAACCTAAGTGCTTTTCTGG
GTTTCCCTTCGGCCACTGAAGCCCACTTGCTTTCTCTCCAGGACTCTGTGTTCACTCGTATAGTGGGTTTCTGGAAAGCCTTGGAGG
TGGGGCTCATGGTCTGGTGAGTATTTGGGGCCAGGACTGCTGTGTGACTACATTACTTTCTGTCTGCAAGGGACACACACGGGTGCC
CTGGGCCACCCAAGAGGGAGTATGAGCATGAGGTCTCCTCTGAGAGATCTCGGGGTTTTCTTAACTGGGACCTGTTGCGAGTGTCAT
TTTTCATATGCTGGGGGATCATGGCTGCATGTGACAAAAGGCACAGTCGACAGACTTCAGAGAGGCCAGCAGAATTTCTACATGACA
GTTCGCTCAACAAATTTCCTTCTAGAGGATTAAGTGAGTTTAAGAGCTCTGTGTAGAGCCGGATGTGGTGACGCACGCCTTTAATCC
CAGCACTCGGGAGGCAGAGGCAGGTGGATTTCTGAGTTTGAGGCCAGCCTGGTCTATAAAGTGAGCTCCAGGACAGCCAAGGCTATA
CAGAGAAACCCTGTCTCAAAAAAACCAAAAAAAAAAAAAAAAAAAAGCTCTATGTAGGTTTTTCCAGGTAGCAAACATGTGTTCATA
GGGTCTCACCATGTTGTCCAGACCAACCTTGAATTCACCATCCTCCTGCCTCAGCCTCCTGTGTCATCACATCTAGCCTCAAATTTT
CATAGACACCCTCCCCTGCAAAGGGGACTGAAAGGTTGTTCAAAAGTTTGTGTATAAAATTCCGACAGCAGGAGAGCTGAGAGGCTG
GGGGCCGCGCTAAGCAGTGATCAGCTAAGTATGGTTTCAAGAGTCTCGCTAAAACACTCAACGGTTACACGAAGCATGCGAGTGTAT
TTTACATTTCAGCTGACATGTGGGAATTGGATGCGACTGTGATTTTAATAAAATAAATTTCATCATGCAGGGAGAAGAACCAGCCTA
TCTCTGAAAGGAAAGTGCCGTCTATCTCCTCATACCAAAGGTTCCCTTGCTCAACAACTGTCACAGATGCTATGACAAGCTGTATTG
TGCAGGGTGTGGGGTTCGTGAGACGCAGCTTTGTGAGGTGGCAGGTATTGAGCTGTGATTTATGTCCCATGAAAATCCATCAGCCAT
TGTCCCACTTCCAGGGCCGCCGACACTTAGGGTAGTAGCATTGCCTCTGTCCAGGGCGAGGCCAGAAAGGCTTCATTCAGCATGGAG
GTGCGGTGGTCGGTCGGGGGCAAACATAGCCCATTTCTTTTCTCAGGGAAGGTGGGAGGATGAAGGTCTGGGGCTTTTGAGAGAAAC
TTTTTTTGTTGTATTTAGTGTGGTGACAGTACAAATGAAAGAGCCTTACAGTTTTAAGGCACCATTAATTTTAATGTGGGGATCTGA
GCAAGTGACAACATGAAGCTAGATGCCTCATTAATAAATGTGTGCGAGGTATTTACCCTCGTTAAGGTTTTGAGAATGGGGATGAAT
GGCAGACAGGGTCAGGAATGGGAAGCAGGAATTGGTTCTAAGACGCGAGGACCCAGGGAGGTCGACCCAGCTCTGTTAGCTTCTGAT
CTCACTTCGTTCTGTTGAAGCTCTTCATTAGTCTCAAGTGAACTGGCAGAATATTAATATGTACATATTAATAGGCTGAAGAACAAT
GATCAAAGCGGCCCTGTTTATATCTCATAGTAGGCGGTGGGCATTGCTATTGTTTATGTGATCCTTGGAAATAATAGCTTTTGTTCT
TCCTAAAGACTGTCTTCGGGATGACAATTTGACAGAGACGATAGACTTTGCTTCATAGGCAAGCACTAAGTTGTACCCTTGAACGCA
GAGTGGCTGTGATTATCTGTGCAAGCTGTCAACAACATTGGGGCTACTGACTCTACCCCTCCCTCAGGATTTATGGGGTTTTCTTCA
GTGGTGTACCTATGCACTAGTAAACAGTCCTAATGAAACTTGTGGGGGTCGATCTCTCTCTCTCTCTCTCTCTCTCTCTCTCTCTCT
AACACACACACACACACACACACACACACACTCACTCTCACTCTAGTTTGTCCTATTTCACCGATTGTCCCTTCGTTCATTAGGGCC
TAGGCTTCAGTTTCTAAGTACTTCCCCCTTTCTGTGCATCACTTCTTCAACCTAGGTGTCCTTGAAAGGATGAGATCTTGCTGGGCA
GTGATGGCACATGCTTTTAACCCCAGTACAGGAGGCAGAGCAACCAGGGCTACAAAGAAAAACCTTATCTTGACCCCCACCCCCCTT
GTCTCTGTGGCTGGAGACAAGGGACTGTGATTAAGAGCATTTGCTGCTCTTCCAGGGATCCAAGATCAGAACACCGTGTTAGATTAG
ATGGCTCATAACTGCTTCTGTCTCCACTGGACTGATACCTTCTTCACGCCTTTGTTGGCACCTGCACACACAAAGCACGCATATACA
AGCATACACACAAACGAGACCAGAATGCTTTTAATAGACAGAAGAAGGCACAGCTCGCTAACCTCCCTGTTCCCCCGGTGGAGGATA
AGACGCCTTCTGCTGCTGTGTGCTCTTTGTGCCTCTCCAGCCTGATTTTATCCACTCTTCCTCAGTCTCCGAACATTCCCAGCCTGC
TGAGCCCAGGGCCTGGTCTAGGCAGAAAGAATCAATGGTGAAGGACATGAGTCCTTGTCCTTCCTGGAGCCACTTTCAGGGGGCTGG
ACTCATCGTTCCCTGAACTCAAATGCTCTTCTTGCCTTAGCCCGGGGACCTGCCCACACAGAGTCTGTTTGAGTTTTAAGTCAGGAC
TGGGCAATGGTGTCCTGGCCCTATAGAATTAGTTCTCCATGTTTTGTGCCTGTGCAGCTTACCATTTAGGTCTGCCCTCAGATGAGT
CTGTTGTGTGAAGTGTCTGTCTCTAAGGCTGCCTTTTAGTACCCAAGTAATGCCCACTGCTAAATGGTCACTCATAAATGCCTAGCG
GCAATGGGAGTGGCTTGAGGTTGCTATCCATCAAGGGTGTCTTGTTTTCTTCTTCTGGTGCTGGGAACACACACCCCAGTACTTTAC
GCGTTGCTTCTAAGTGACTTACTCCATTCTCAAAAAGAGCCCTGTCATCATACATGCATTTTGACAATTGGACACAGGGAGGCATTT
TGATGAAAAAAAAAAAAACCAAAAAACAAAAAACAAAAAAAACAAAAACAAAGCCAGACCCTATTCAGACGGGTTCTAGTTCAGTTT
GTTAGTGAGAACTGCCGTTGCGGGTTTACTAGAAACAAACATTGACCAGCCATGTCCTGTGGATACATGCAGATAAGGTCAGGATCA
ATAGTTGAGACTTGCTTTACAGAAGTGTCCTCTGACCACAGTGACAGTGGCACACTGTGATGGCATTACCCCCCTGTATGTTGGTTC
AGTGTCATCTGTATTGTGACAGTGAAACTGTGTTGGGCGGTCACCGTCACATGACTTTTAGTAAGAAATCCTTACCAGGGGGATACC
TTGGACTCTGTTTTTATAACCTACTCAGCCATGACCTCAAACAGGCTCTGACTGTGGACTTCAGCAAGCTGTCAGCAGAGCTTCGGT
TCATTCAATTGTAAATGTTAGTTCATTTGTCTTCTGTGAGATCATTGACTTAAAAAAAAAGTCCTTATCATGCACTCTTGCAGCCCC
CTGCCCTTTCCTTGGGTTCCTATTGACAGATCTGTCTGCTCGTTGCTATTTCAGTGGCATTACCGAGTAAGTGCGGATCGTAACTGC
ATTTAGCTAGATTCTAAAGCCGTGACTATTGAAAAGACCCAACCCTTGTCTCAAGGGAGCCCATTTTGTTACTGTTTTCTGCCTGTT
TGGTCAGTGGATAGTGTTGTTGTAAATATTTAAATTCAATCTGGGTTTCTACCCAGCCTTTGATCATTCAGTTCCCAGATAAAAGAC
ACACAACTTTTATATTTATAATGAACCTTAATCAGCACTAAAGCTTGGCAGATGTCTACCTTTTCTATGCCATTATTCTACTTCCCT
GCCAATAACCCCAAGATATGACTAGCCATGTTCCACCTGGGCTGCTCTTACTCCAGTTGTCCAGCCCTCATGGCCATGTTTTCATGA
CTCATCTGCCCCATGGGGTCTTCTCCTCTCTTATCTTTTCTCCATTCTCCTCCTTAATGCCTGTCTCAGACCCCAAACCTAGGAACT
GAAACCCCACCTACCTCTCAGCTATAGGCTGTTGGCATCTTTATTCAGCTAATAGTTTTAAATTAAGGAGCAATTATGTGGCACATC
ACTTGTTCTCTGTCCCTGGGGCCGCCAGGCCTTGGGGTCTAACATTACAATACATAGCAGAAGACCAAACCTCAGCAGAATAGCTAT
CTTCCCTGCTGTGACTGAGGGACCCAGGCACTTCTCTGGTGTAGTTCTGTTAGTAATGTTGGGCTCTGTTTTCTGTAGAAGGGGGAA
GAGGAGAGTGTTCATTTGCCTCTTGCTGACCATAGACAAAAAACAGTTACAAGGCCTTTCAGACTTCAAGAGGATCTGAGGCATGGG
CTCACTGGGTACCCCCTAAGGGGCAAACAGCTTTGGTGGACAGCTTAGCCATACAACCCTCTGAACTAGATGTAGCATATAAGTAAT
AACCTCTGTGTAGACATTTCCATCACACGAAGGCATCTGCAGAGTTTGCACTGTCACATGACCTAAATCCCGTCTCATCTGAGAGTT
TGCTGAGCTTTCGACCCCATTGCACCGTCCTTTCCAAAAGCTGCTGCAGCTCACTCTCTGTCATATAGTTTTATTTTATCTACTCGA
CTAGAGCGTGATACAAGCAGACCAGATTTGACTGTCTGACGTTGAGCCTAGAATCATGTCTGGCACTTGTCTTCAAGGATTCTTGAA
TTTTTCCAGTGGTAGATTTTATTTGTCATCTCTGTGTCAGTAACTCCTTGCTACCCAGCCCTTGTCATAGGTAAGTTACAGCTACCT
GCTGGGTCTGGTGCGAAGACCCTTGCTGTCCTTAGACGCACCTCTGTTCTGTAACCCATTCAATGGTATTTCTGTGCCGCTTGCTTG
TAGTGTGAAAGAACTAGTAAGTAGCATACTTCAAAGTCTCCTTGTAAGTAAATGTATTACTCAGTTATAGGAAATCATGATAACGTA
ACGGAAATATTCTTTGAGATCATGGAGGAAGTTTTTGAGAAAATAATCTCTACTCCACTTGTAGCCTTTGAAAAACTCTGCTTATGA
GTAAGGCAGTCTCTTCATTGCTAATAATAATGGCACTGGGGGAGCTGGAAGGAAGAGACATTTGTCATCCAATGCTAAGTTACCAGC
TTGTCTTGTTGTTTGGGTGGAGCGCTAGTCGTTGGGGAGCAGAAGCTCAATCTAACATCTTCTTGGGAGAAAAAAAAAAAAAAAAAG
AATAAAGCCACCATTTCTATATCAGGGTATTGTGGATTGAGACAGCAAAAACTTGCTTGAGGAGAAAGCTTAAGCTTGTCTTGTGAA
AAAAATTCAGTGGCATGTTGGTGGAAATTCCTCAGGGAAATAATGTCTCTGGAGGTGGAAACATGGAACTACTGCATGCATTCGGGA
GGTCATAGTACACTGAGGGGAAATAAAGTGATCAGTGGTTATCTTAGGATCTTATTGCTGTGAAGAGGCACCATGACCAAGGCAACT
CTTTTTTTTTTTTTTTCTTCCTGGTGCTGGTATTTTATTTCTTTGTGTACATTAGGCCATGAATTCGTATGGAATGGGCTCCAACAG
CTCAGGCTCTTTTCCGTTAGTCCTCACAAAGTGTGCTTCTCTGGGTGGCACAGGCTGGCGCTTCAGCTGCACCCAAGTGCCCTTCTC
TTTGGCTTCCTTTTTCTTCTGGTCGTTCTCCTTCACCCACTTCAGGAAGCTGTCTCTGCTCTTTGAGTGCTTGATGTGCTCAATCCG
CACATTGATCCTCTTGGCCAGAATTTTGCCCTTAACCTGCTTGTTGACAGTGATGCCCACGGCATGCTGGATAACATTGTAGACTCT
TACGGTTTTGCCATGGTAGCACTTATGGGGCATTCCTTTTTTAACAGTGCGCATTCCCTTGATGTCTACAATATCACCCTTCTTGTA
GATCCGCATGTATGTGGCCAAAGGAACAACGCCATGTTTCCTAAAAGGCCTAGAGAACATGTACCGGGTGCCTCTCCTCTTTCCCTT
TGTGTTCCGTCATTTTGGCCAGTTACTGGAAGATGGCTGCCCCAAGGCAACTCTTATACATAAAACATTTCATTGGGGCTGGCTTAC
AATTTCAGAGGTTCAGTGCCTTATCCTCATGGAGGGGAGCATGGCAGCATGCTGGCTGACATGGCACTGCAGGAGCCAAGAGTTCTG
CATCTTGATCCACAGGTAGCCAGGAAGAAACTATCATCCCCACTGGGTGGCCCTTGAGCATATAAGACCTTAAAGCCACGCCTCTAC
AGTGAAACATTTACTTCCACAAGGCCACACCTCCTAATAGTGCCATTTCTCATGGACCAAACACTCAAACATTAGTCTAAGGGGGCC
AAACCTATTCAAACCACTACGTTGGTGTTCTGTGGGCTGGGGTGGCTGGCTGAGTCAAATTTCTGCTTTCTACCAAGTGTAGAGACT
TCATGTGAGCAGAGTGGACTCTGTCTGAAGACAGACCCAGCGGGAAGATGTGTTGGTTTGGTGTTTGGGGTGTTTGCAGATGCAGAA
GACCATCAGGAATCTCCCTGTGTTGTAAAGTATTTCCATCTACTGCCTTTTAAAGGGGTTTCAGGCAGAAAACAGAGCCTAATGTTA
GTAAAATGGGAAAAAAGCTGCCTTAAAGTATCATTCAAGAAAATAGAGTGCACACTTAACGTTTTTGCATATATAAAGGGTTATAAA
TCCTCTGGGATCTCCTAGTTGCTAATTTATTATCCCATGCTTACTCATTGATAAATTTTTCTTCTTGCAAAAAGATAGATGAATAAG
GGAGAATACCAATTTTCCACCTCATAATAAAACTAACTCATTAAATTATTGCGTGTGGGCTTTTGCTTTGTTTGTTTTCTTTTTCTT
TTTTCTTTTTTCTTTTTTTTTTTTCTTCTCATGTTCTGGTTGGAAGCTCAGGCCCAATACATACTAGACAACAATGAAACTTTTCCA
CCCCCTGGAAAGTTAGCTGAAGTATCCATCGTGAATATACAGTCCTATTTAGCCACTTTTATAGTTTAAATTATTCTTTAAGATTTC
CAAGTTTGGAAGGTTGGAAAGAGAGAGTCTGATGGCTTAGCCCCATCCCTGCCATTTTGCTACAAAAACTAAAAAGAGACCTTGACT
TTCTGTGGTCTCAGATTGTTCCATCTGCACGTGCGTGAGTGAGTGAGAGTGTGCGTGCGTGGGTAGGTGCGTGTGTGCGTGGGTTCG
GTGCAGGATAACTCGGAACATATCAATCAAGCCAGAAAGAAAGCAGAGCCCTGGGTAGGGTTCTGCCCTTCCACGCTAATGGGAAAA
ATTGGTGCTTTCTCTTTGCTCATTCTGTGTCATTCTTACTAAAGTGTGGGTATTTGCAGGGCTGTTTACCCTACTTTGCCCACGGCT
GCAGATAAATTATTATTTTGAATGTGTTGTCTCGGAATCAGAGAAATCCTGCTGCCTCACATTTGGACTGTGGCCCCTGCACAGTGT
CATGGACTTGACTGTGGCCCCTGCACAGTCTGATGGACTTGCCGCACTGGGGCATCTCTGCTTTGCTGAGACTCTCTGACATATGCA
GGTGCCTGTGGATGTGGGAGACTGATGGAGTAGCCAGAAAAGCAGAATAGAGACCAGAGGAACCGCCAGCTGGAATGTGGAGTTGGT
AGAAGGAAGTTCTCAGAGCATTTTGATGCTGTAGATGTGTCAACCAGGAGTTGCAAAAGCGGCTCACCTATAAAGGGCCAGCAGACT
GAAACCCCCAGTGAGGGCAGAGGGTGTCTGCATGCAGTCCTTTCCAATGGCTGCTCGGAGCCTCTTTCCACTTCCTAACACTAATCT
TAGTTCTGAACTCTTGTTAAAGCAGAAGATTGGAAGCTGGGCGTGGTCGCGCATGCCTTTAATCCCAGCACTCGGAGGTAGAGGCAG
GCGGATTTCTGAGTTCCAGGCCAGCCTGGTCTACAAAGTGAGCTTCAGGACAGCCAGGGCTACACAGAGAAACCCTGTCTCGAAAAA
ACAAACAAAAAAAAAAAAAAAGCAGGAGATTGGGTAGCAGAAGGTTAGAGTACCCAACTATGAATTATAGAGAAATGTTTTATTTTA
TTCCTTATCACTCTGCAGTTGAGCCTAGGGCCTCATGTATTGTAGGCAAGTGCTCTTTCTGTGAACTCCAGCCCAGCCCATTTGTTC
TCTTGGATTTGGGATGGGAAGTTCTTCAGGCGCATATACCTTTTATCTTTGTCAGCTTGCTTTTAACCGAAGTGGTTGAAAGTAAGT
ATCTGAGTGATTGACAAAATGGCCCAAGCCCAAACGATTCTAAGGGAGGGTTCTGTGCCCTCTTGTGGGTTTCAGCCAACTAAGTTT
TGATTCAATAAAATCCTCACCCATAGATGAGTGGCATATTATACTATAACAAACATCACTAGCTAGCATGGGTAGGACTGGGGAATC
TTTTGCCGTCATTCAACTTAAAATTTTGGTTTAAAATCAACTTAAAATTTTGGTCTGCTGTGGACATGTCACACTTGTGAGCATGCA
TACTTGAAGGTTTAAGCTTGAGTGTTTAATGACGAGATTTAAAAAGTGAAGAAATTTAATTTTTCTCATGTGTGGGTATTAATTTGT
ATGCATGCTTGCACACACATGGGCACTCGTGGGTGAATGTGGGTAAGTATGCCTCCATATGGACATGGATATAGGGCCTTAAGTTTG
CCGTTGGTATTTGCTTTCTGTAGTGTCCCTCAGTCGAACCCAGAGTTTGTCAGTATAGCTAGTCTTGCTAGCCAGCTTGTTCCCATG
GGCCTCTGCCTCTGCACTGGAATTACAGGAGTCACCATGCCCACCCCCATTATGTGGTGCTAGGGGTTCGGCTCTGGTCCTCTTACT
TGTGTAGCAAGCAATTGAACTACTGAGTTCTCTCTCCAGACCCGGACTGAACAACTTTGAGTTAGAAACCCTTTTATGTTCATCGTG
ACCTTGTTTGCGTCCCTTTACCTGCTCTGGTTGAGTTATCAGAAGTAACAGCAGCCCAAAAAAATCCATTGCTCTCTTACAGTCTCC
ATGAACGCCAGACATACACATTGTACCTAGACATATGTGCAGAAAATAAAAACATACACATTAAATAAATAAATAGTATTATTTAAA
TGAAGATCGGTCTGAAGGCTGTTGCAGGTCCTCTGTGTTTGCCGAGGACTTGTGCTGTCTTCATTGATTATCATATTCACAGTTTCT
GAAGAAATTACCCCAATATGCTGTAGCTCTTCCTTACAAGTTAAAGCCAGCCACCAAAGCACAGTGGGTGCTGAGCAGTGAAAAAGT
ATCTTCTCTGGAGTCTGAGTCCCGCATCCTTTTCGTCGACTGCACGTCCTTCAACCCTCACCTTCTCTTTTAGAACATTGAACAACT
CTAGTGTGAGACGCGTGCTTCCTGTGTCAGTAACTAAACACGACTTGGCAACTACGGTAGGTAGTACCAAGTACCACAGACCCTGCT
ACTCCCGCACAGATGCGGGTGATGTAGAAAAGGTATGCTTCTTCTGAGCGCTGAGAGCGGGGAGCCAGCATCCTGACTGTCCTGACC
TTTTCTAAAACTCTGTTGTGAAACTGCAGCTCTGCAGAGCCTCATGTTTGTATGATGGTTTGCATCATGTCTGCACACAGGTTTGTA
TCATGACACACATACATTCATACTTACATACCGGAACATGCATCCCTGCCCCCTCACCCCCCCCACTGGGCGGAGGAAAAAAATCCC
AGATAAAGTACCTTTTTTTTTTGTCCTTTTTATGACATTTAGTTGGCATGAGTTACCGCCGTATAATCATCTGTCTAGTAGAACCTA
ACATGTTTCTTGGCTCGAAGTCCCTCTTTTGTCCTCTCCTCCTTTCTGCACTCTGTTCTTAGAGGGAAGTCACTGTGCACACATAGA
CTTAATGAATGGGAACTGCCCCCTAACCAACTTGGGGTTCTCAATAAATTACTTGGAATTCTGTTATATTTTATGTATGTGGCTTTA
GCAATAACTTTGATAGCAGAATGCTGCTGTAACTGTTGTAGTTATAAACTCACTGCGGTGGCCATTTTATTCTAATTTGAAGGGAAC
TAGTAGCAGCCAGACAGCCCTGAGCAGAGACCGTGGAAGCAGAACTTCTACCAGGGATTGTCTTACTGTGCCAAACATGCCAGATGC
TGTCCACGCCTGTTCTGGGTCAAGACCCAGGTCTGTTTGTGTGCTCTCTTCTGCTTCAGCAGGCTCACTGGCATTTCTGGAGACCAC
AGCTTGTGTGTCCTATCTGCATTTCAGCGGCTATGTATCCTGTTTAGCAAATGCTAGTAGGAAAACATATGCCATTTTGACATGTTT
AGGTATTGCAGGGACATAGTTGTTAATGGGTATCTTTTATATCCATTAATTGCCTCTGACTTGAAGTTACTCCTCATAAGAGTAATT
AGGATATAATTAAAGATCTTCAAATGTAGTCAAATATTTAATTACTTCATATTCAGCACCGTGTGTGCAGTTCTTGGGTTGTTTTCC
TTTTTTGTTTTCTTTTTTTGAAAATATTCCAATAATTTTTTACAAATTTATTAATTAAGGATTATATTTCACTCACTTTTGGTGAGC
ATATATAGTCAGTGAGAATTAGGTATATTTTGAAAGCATAGAAGTCTACGTATTTTGCCACTGGCTTCAGCAGAGAATTAGTAGCGA
AGTCACGGTTTTGCCCTTGCTCAGTGTTTCATGGATGCGCTTCTAACCCTGCCGTCAGCCACTGAATATGAGTGGTGATTGTTCTCA
GCCTCAAGAAGAAACGGAGGTTAATAGAAGTAAATTTCCCAGTTTTAAGATTTTTCTCCTATCAATTTTCCTTAAACTCTTGGCATC
TGACCCTACATCCCTGGTGGTGAACATGACTAAAGCAAATGAGAAGTAATGTAAAAGTTGTTAGCAGAAGAATTCCAAGGCAGCAGG
TAATTCAATAACTCCCATTATCTTCTTGAGAACACAAGTTTATACTGTGGGGGTTTTGGGTTTGCCCTTTATATATTTTTCATATAC
TGTCATTTATGGGAAAGAATCCTATTTAATAGACAGACACAGAACTATCGCCATGTTCCCAGTGTTCCTCGATCCTCCTCCAGGCTG
GCTTGGTATCCAGGTGACTCATGCTCTGAGCAAGGTTTGAACGCCGCCCCTGCTGAATGGAAGGCAGCTAGGAACCCCCGAGCTTCC
TGAAACAGGGAGGCCATCGTCCAGCGGTGATGTAATTGTCATCTGTACATCCTTCTGCGGGAAGGATGGGTTCACTTTTCCACCCAC
AGCGTTGGCACACAACTCCCCTGCGTCTCAGGCCAACACTGTGCAACAATTATGAGGTCCTAGAGCATTCCCGGCAAGGCCTGTGTG
TACATCCTGTGTGCAGGACACACATGTGGCTGTAAATGGATGTGCTTGTGCCCAGTAAGTAAGTCACCAGTCTTACTTACTGTATGG
GAAGGATGAATTGATAGCATTTCTGTTTATGATAGAATTAATTAGAATAATTAATCACCGTCCTCCAAGGATGTGGTAGGTCTTTTC
TATTTAAAGGGGAGGATCGGTTTTCCAAGTTTTTTTGCTAGGTTTCTGTTTTGTGTTTGTTTTGACAAGGGTCTTACTCCATTGCCT
TCCAACCTCAGTTCCCAAATAGGTAGGACAGGACTTGTGTGCCACTGAGCATGGCGGATAAGGTGTCGTAAGTGGCCAGAAAAGGCT
TATTTGAGGCACTACGTGAAATGAACCTCAATTAGTGAGACTAGCCCGTTCTTCTAGGAGAAGATCAGTGGATGAGAAGTGGGAGAG
TGAAGGAATGAAGACTAGACCTGGATGACTACAGAGAACAGCACGTTGTGAGGCTGTGTAGGTAGCAGGGCAGAGGTCCAGGGACCC
ATGTGTTGTCTCCTCAGAAAAGAGCATGTGTGACATGTTTTCTCACACTGAAAAGACTCATGAAGCCAACACCTAGGTCAGGAGGCA
GCATTTCTGCTACCATGGGGGCCTTGCTGGATGCCACACCTGTTTCGGTCTTTGTAGGTCAAGTTCACTTACAATCTTGCCTCAGGG
AAAGTTCTGGAGCTTGCAGTCTGTTCCTTACAACCTCTGTGATCTAAAACGTAGTGCAGCATGCAGGGTATTTCCTGTGGTCTTTTC
CCCAATTCTCGCCTCAATCCGATGCCTGGAAGTACGTGGTAAATGTTTATTTGTTTCTATATGTGTTTGTCATGTGTTTTCTCTTGC
TTAGAAAGCAAGACTTGTCCTGTGTATCCTTCCTTCATGTCTAAGCCAAAACTTTTGTCCCATTTTCATGCATATTTAATACTCATT
TTTGACACAGTTTATATTTACTCTTTGATAACAGAATACTTCAGGATGTGTCGTTCATTAAATCCAGAATTTTGTATAAAAAGTCTG
TAGGCACCTAACCAAATTTAAACTGTAAATAAACTAGAGGTTATACAGCAATTTCAGACAGCTTTGTATTTAAGATGGGACTTTTAA
AGGTAGTCCATAATGAATCTTTTTTTTTTTTTATTTTTCCAGAGAGGGTCTCATGTAATTTAAGCAGATCTCAAACTCACTGTATAG
CTGAAGATGACCTTAAACTTTTTTTTTTTTTTTTGTTGGGGCTGGAGAGATGGCTCAGTGGTTAAGCACACTGATGGCTCTTCTAGA
GGTCCTAAGTTCAAATCCCTGCAACTACATGGTGGTTCACAACCATCTATAATGGGATCCTATGCCCTCTTCTGGTATGTCTAAAGA
CAGTTAGTTACAGTGTATTCACATACATAAAATAAATAAGTCCTTTGAAAAATTGTTTGTGTGCTTGTGTGTTTGTGTGTTTGTGTG
CTTGTGTGTGTGTTTGTGTGCTTGTGTGTGTTTGTGATTGTGTGTGTATGCATGCATACACAGTGTATGTGTAGTAAGTATCAGAGG
ACAACTTGCAGGAGTCTGTTCTCTTCTTCCCAGAGATGAAACTCGGTTTGTCAGGCTGGCCACTGCAGCATCTTGACATCCAGGCTT
GACTTTCAGATCCCTGCTGTTACCCTCTTGAGTACTGGGATGAGAAGTGTGTGGGTACCATGGCACCCACACAGTGCTGGGGACCAA
GCCCAGGGCTTTATGACTGCTAGGAAAGTGATCTTCTGGCTAAGCTGACTCCCCAGCCCTAGAGTGAACTTTGATGATGATGATGAT
GATGACGATGACGACCACGACGACGACCTGTACAGTGGTCTTTACCTGGCGGCAGCAACTCAGGTTGTGTAGCTGGTGTGGGAGAGA
ATATAAGCACTTTCCTGTCTGCTTGCTCCCTGTTGTTTCACACCCGGAACAAAGAGAGTGCAGCTAAGTCAGTAGGTTCAGTAGGCT
CATATTGCAAGCCCAATGAATTTCAAATAAAAAAAAAGTTACTAAAATGCTATATGCTTGGCTTGGAACCAGATACCTTAAGTCTTT
TTAGGGAGTTGGGGCAAGGTGCTTCTTTTTATAAGAGAATTTTACACTAGAAAAAAAATCAAGTGTAACTAGTGGTCTATTTTGAGA
CCAACCCTTTAGAAAACGCTTTAGGCGTTTTCTTTGGAAGATCGGATCTGATGTTGTCTTGCCTAAGCTGCTGTTGTTTATTTTGGC
CTTCTGAACTGATGATCGTCCTTAGAAGCAACTTGGGGCCGCCTCTGAAAATGCCAAGATGTGTATTTTGCCACTGAGACATTCCTC
CCATAAAAGCGTGTGAATAATTTTTTATGGAGCGAGTCCATTCTTACGATGTCTTAAAGGGGGAAGGATATCATTTTCAATCTAGAT
CATTACCTTGTTTCTTATTTCTGAATGCTCATGCCCCCCCCCCCCCCACACACACACATTATCTAGATCGTGTGATTTAGATGGACC
ATTTGGAAAAGCTCTCTCCCTGCCTTCTCAATTGTTTTTCTAATTTTTGAAATAGTGCCACCTAGGGATAACAGTTTTAAGCCACCT
AACATTTAAAATGCATTTTAGTGTTTTTGGCCTCACCTTTTAATTTTCTTGCTTCTTGGAGAGAAAGAGTTGACTCTAAAATGCTAT
TTTATCGGTTCAGCTTTTATTTAATTTACTCTAGTACGTGTAGATGGAAAATTAGGCGATTTAATACTCACAATGTTATCTGGCAGT
TATTTTACTTACTGTTCCGTTTTAAGATGTTAGTTCGTAAAATACAGCCTTGAACTTATCTTTCACATGAGATGCTGAAAATGTGTA
GTGTTAATTAAATGGGCTCCGTTGTCGGTTAACCGGCATGTGGGTACATCAGGAAAGGTACCAATGGACAGGTCACGGATCATTTTA
CATCCACCGCATACGTGAGAACGCCACATGCTCCAGCTGCTCTTGGGGAGGTGAGGAATCAAGAGAGCAGAGGTGACTCTGTTCTCG
GTGCATTCTGGGAGATGGAGGATACAGGCTCACATGCTAGAAATGCCGTATGCCGTGGTCTGTCTGAGTTCTAATGCTCTGTCCATT
CCTTGCCTTTGCTTTCTCCTGATTAGCTTCCTTCTTCGCCTCCCCTGGCATCCCGACAGTTTAACATCTCTAACAGGCAGGTGTAAT
ACTTGACTAAGAATCTCTGGGTTTGGGGAAAATCCTGGAACGCTTGTGAAAAAGCCAAGCTTATAAGCCTGCGTGGGGAGGTGCAGC
TATGCGCCTGTGTGGGGAGGTGGAGCTACGCAGAGTCCTGGTACTTGCCTGCTTAGCTTATTTGGAGAGCCCCAGGCCAGTGAGAGG
TATTGTCTCAACTAAGCACCCTGGAAGATGCCTTCTAAGAACTCCAGAGATGATCCTTTGCCTCTTGACCCATACAAACATGCATAC
CCACCCCTTACACACACAAACACACACACCACACGTGCACACACATACTTAAGCATTTGTACTTACAAGAATCTCAGGTTGGCCTCT
CACTGCCTTGGTATTCCCAAGCCGAGCCCTGTAGCACGGTAGGTAAGAGGCAGTGATCACTAGGTCTGGGTCGTAGAGCTGCCATTT
CATGCTGCATCTGAGCCGCCAGCATCATGCTGCCCATCAAGAGACAGGAAAAAGGGAAACCGACTTGTAGAACCTACCAAGCTTCAG
TGTCCAGAGAGAGGATCGACAAGGGATGAGAACTGATTCATTAAATTATTAGCTGGACCATTTAATATAGCATATATACTATATCAC
ACACATAGTTGTGTGTGTGTGTGTGTATATATATATATATACTTACATATGTATACGTGTGTGTATATATACATATGTATATATGTA
TATATACATATGTATATGTATGTATATATAGTATATATATACACATATATACATGTATGTGTATATATGTATATATGAAGTATATAT
GCCTCTTGGCACCATCCTATGCCAAATTCTAAATAATTTGGACTTTAAATACTTAACTGGTCATTTCTGAAGGGTAATAGACTTAAA
AATAGCTTTTTTCCCTTTGTTTTGCTTTAGTATCATTGAGAGAAAATAACTTCATAAGTGGAAAGACATTTTAACCCCCATGAGTAT
GTTTAACATTGTTATACATGACACGGAGCAGGGCATACAGCGCAGTGAGGGAGCACCTGCCTGCTATCCATGAGGCCTTGCAATCCA
TCCTCCACACCATCTAACAAACAGCCACAAGTAAAACTGAAATGGGTTATGGATTAGAACCCCTTTTCTAAGGGGTTATGAAGGGTT
TCTGGCCACGTGTCTCTTAGGAATCCTACCAAGACCCTCTCACATGCTCACACCTGTGAGGACCTTTCCTGAGAGAGGTGCTGATCT
GTCATTACATGTACAAAGAGCCATCCCACAAATGGTATCCAACCACCGATGGCTAATGCCTTTTGTCGCTAGAGAGGAAGTGGTTCC
CCTGTCTTCCGGGAGTTAAATAACTCTCAGGTGGGTGTGATGCCAGTTAGAAAGAGATTGAGCCTGAGGATGTACTATTGGCTTTGT
TAATGAATTTGGTTCATACGAGATTAGAACGTAATGTAATATGGATGAAGCTGTAATGAAATGGGCTTCCTTGGCCCTTCTAGTTTC
ACGGGCAATGGCTGGAAAATCACTGCCAGTTGATCTTGGCTCAGAGCTCGGACGCAGGCTCAGACCCACCTCTTGCTGTTTGGAAGA
GAAAGGAAACATGATAAAATATATGGCTTTACTCTCAGAAGCAATTTATGACGGCCATTTAATAAAAATTTCAGGAGCTTCCTTTTC
TTCCGAGGAGGAGAGCCAGGGAGTCCTGGTGGGACCTTAAGCTTAAGGTCCCGTGTGAAGGCCCTCCACAGCCTCTAGCCTTCCCAA
CTTCCTGCTTGGCTACACAGGAAAAGATGGAGGGATGGGTGGGGTGGGGGGTGGGGGCTGAAGACCTTTAACTCCATCAAGGTTCCA
GTTCTGCTTATACATCTTGACCCCCCCCCCCCCACTCTGAACTCAAGCCTTCCTGGTGAGTTCTGACTTCAGCTTTGTGGGTGTGGG
AGGGGGCTACTGAGAGCCCCACTCTTATACAAACTGACACGTGTCCAGCTTGTGTCCAGCCCACTCTGTCTCGTCCCGTGGTTCTTT
TTCAGCCTTGAGTGAAGCAGTATTTATCATTCAGGATCTCCATGATCAGCAGATACCAAGGACCTGGAAGGACCTGAAAGGACCTTT
GGTCTCTAATGTTTACTTACACCAAAGGGCCCTCAGGAAATTCTTAGGTGCGTGAGTTTAAGGCTTTCTCCTGGCAAGCGGGTTGCA
GTGTGGCTCACTTCTGCCATTCCTCTTAACCTGGGAGGATGTTTGTTCTGGCATCCACTGCCTGTGCCCGGTAACCATGGTTTAGAC
AGTTTTACAACCAAAATTCACACAGCTTCCTCTTCTGCCTTGTATCCTTCAAATGCTTCCTGACAATGACCTATAATGAAATATGCC
TTCTAGCATGGCACTCAGTAAGCATAGACATATGTACATTTCACACAGTACCCTTATAAGAAATATACATACCATATTCTGAGAATA
TATCTCCTTCTGTGACTAGCTCATGTTTGCAACCTGCAGTGGGAAAACTGCCCCCAGGCACTCCCCCACCCCCCAGAGATGCTCTGT
ACACTGCGGTCTTTTAGGTCATTGTAGTAGAGGATGGCTTAGGGCTAAACGCTGAGCATGGCAGAGCCAAGTGTGTGGCCTTTCAGT
GGCAGCCGAATGTTCTGGAAGAGTGGCATCGAGTGTCCAAGACCAAAAGTTCTGGCTGTCGTGTTCTGTTCCATCTGGGCCCTGACC
TTCCCATCTGCTGTGAGTCTCTCAACCTCCCTGAGTCACTTCCCCCTCATGTACAATGAAGGCATAAAACTTTTTACTCTCAAATGT
TGGGAGGATTGATATATCATCCCTATATTAATGCACAGATATTAACAGCATTTATAATACTAATAGTCAACATTTATTTAACGTCAT
GTATAGCTTTTCTAGGTTAGCAGACACTTGCTGTTATAATGATTAATGCTGTATCTTAATTTTAGACACATCTCTAGTTGGATGGCA
TTTGCAACCCATTAAGAGTAGGTGGCTTACGTGTAGGGATAATCCCCGTGTCCCAAAGCGTCAACTCTAATCAATGTGATTAAATGG
AAACAATAAGTTGCACAAGGTATTTACAGTCTAAGCTGGTTGAAACTCCCTGTTACATTCTGCACACTGACGGACTTATAGACTAAC
AGAAATCTCTTAGTTCCATGATGGAAGATTCCGTGAGCATTTCATTTGCTATTGGCCTTATTCAAGTGCCTCGGTAATGTTAGAAAT
CTTTCTCATCATTTCTTTTGCTGCCTTTCTATGTTGGTTTCATTTCTAGGCAAGCTTGTCCCCAGGTAGTGCTAGAAACAGCTCTCA
GCTACTCAGTTTTACATTCTCCAAGCAGAAAAACAAAGCCCCAACATGCTAGTTTTAGCTTAGTCCTTTGATGGGCAGAGTGATCAG
CTAGATGAAGGCTACCCCTTGCTGGCCTTTTCAGACACTTGGCTAAGAGAGACAGTGGCTGTTGGACACCATGCAGCATATATCCCC
CCTGGGGGCTCAGCCCATACTCAGAAGCACGGAGTACAAATGGAGCGCACCACTCTGTGGGGATGTCCCAGCAGACACCCGTGGTGC
ATCAGGCATCTCCTTCAGTTAAGCTTAACTTTCTTCATAAGATAATCAGCTTTGAATAACCCCCAACTGGGCAACACTAACACCAGA
GACCTTATTGCAAACCACCCTCACTTTCATTCTTGATGTACACTGCGGTAGTGCCTGAAGATCTGTCATCCCTTCTTTAACTTCCAG
CCCATCTTTGTGTCGTCGTGATTTAGAAGTTATCCCTTTAATAGTGGTTGCTTTCATTATGAACCACAAAACTCAGAGGTGGACTAC
CCCACCTCCTTGTGGTTGCTCCCTTTCATTTTGTGATCCAGCTAGCCCCAGGCCAGCCCTGCTTTATGCAGTTTTCCTTTCATCCAC
GGGCTGAGCATAAGTGTAGTCTCTCATTTATTAGTGTCCCTTGCGACCATACATACATATGTCTCTCTCTCTCTCTCTCTCTCTCTC
TCTCTCTCTCACACACACACACACACACACACACACACACACACACACACACACACACACGGCAGTGAGTGCTTTGGGTTTAGTGGG
GCCTGCAGATGGTACAATATGGTTGACAGGGAATTTAAGAGAATTATGAGTCTAGAGCCAGAAAAGTTCTGACAACACCTTAGAGCC
TTTTATCTGCCATGTGAGATAATTAATTAATTAGATTAAATTTGGAACAAAGCAAGCATACCTTTATTCCTAAGACAATATTATGTT
GCATTATTTAAAACTGTATAGAATTGATAAGTCAAATATTGCAATGTTTTATATATAAGCAAAGCTATGAATTCAGATAGTTTATGT
TATTTTGGGGAGATATTGAAAATTCTCTGAAGATATTAAAAATTTCAAGTCCTGGAAATATTATATGGTCAGGTTCACGCATGCATA
TACATGCATCACATAAGAAGTGCCAGGTGAGCCAGGCAGTGAGGCTGTATGCCTTTAATCCTAGCACTCAGGTGGCACGGGCAGGTG
GAGCTCTGAGTCCGAGCAGCTTGGCCTACAGAGCAAGTTTCAGGATACCTAGAGCTATACAGAGAAACCCTGTCTTGAAAAAAATCA
ACCAGCCAGGGGGAAAAAATGCCAGGTGAGACACATGTGTCAGTTATGCTTTGTTTTCTTATGGGAGCTTGGGGGCCTAATAGTCAT
TCTATACAGCTAGAGTTGAAGTACAGTTGTTACACTGCAATTACGACAGGAAATGGGTGAGTTAAAGTGGTGAGGCCACATTTAAAA
TCAAAGCCATATGATACATGTTTATTTTAAAAGTTCCATGTTGATTAGCATTTTGTTTTCCCTATTACTGTCCTGTAAATGGAGACC
TGGGTGTTCCTTCTCCCAGTGATCCGCCACTTGCTATAAAATTATAAGCACCAGGAGTGTTTGGATGTAGGTCTGTGTGGCCCCTGA
TTCAGTCAATGCTTCCTGTATAAGCTTTAGCACTAACTTATCCACCACAGTGTCCATTTACAAGTCTACAACGTGGGTCTAGACCTA
AGAAGCCTTTATAATAAGCTGAAGATTTTCCCTGCATTTTCCTCTAGTTCTTTATGGTTGACTCTCACACCTGATTCAACGGCAGAC
TGTTTTCATGGGATCTTTTATAGTCAACAAAAGCGGTAGTGGACCAACTTATTTGAGTGGGAAAGGTAGACCAGGCTGGCTGTGTTT
AGACATCAGTAACATCAAGGATTCCTCAGAAAGAATGGTGTTCAGTTTGCTGTTATATGTCCACATCTGCACTGTAGCATCTAAAGC
CCCGTTGACATGGTTTTCAGCCCAGGAATAAAGCAACATTAAGAGAAAGCCACCACTTTTCACAGTTTAGCCCTTCCTTGACCTGCA
TCATGATTTGATTAGACGCGTCCTGGACAGGCTGTGTTGGAACACTTGGTCCTCAGCTGGGGTGCTGTTTTGTAGGCCATGGTGCCA
TTGGGGGGGTAGGGCCTAGCTGGAGGAAGTAGACTGCTGGGTGCTGTATCTGTCTTCAGAATTGACTCCTGCTTCCTGGTGTACCCA
GCCCTGAGCTGCCTGCACTGCCAACAGGATTCCAGCTACTGTGGCTCTCTGCCTCAGTGGACAGCATCCCCTCAAACAAGGAGCCAA
GACAAATGCCCCACCCTCTCACTGGTTCTCTTAGATGCTTGTTACAGTGTGAAAAATGCAGCCCAGCTGGGAGGTCAAAGACTAAGT
CCACAGAGAATGAAGTTTATAAAGCCACTCCATCTGGTTTCTTGTTTGCAGACGTAACTCACGCTGTCCTAGCTAGAGAAAGCCGAG
AGAAGAATGTGAATATTGATATGTATTAGTTACCTATCACTGTCTTCCAGATCACCCAAACCCTTGCACTGTAAAGCAAACTTAAGG
TCTCACATGGTTTGAGAATGAGAAGTCAGGGAATGGCCTAGCGGGCTGGTCTCACAAAGCTGCAGCCAGGCTGTGGGCAGAGGCTCT
CTAGTCCTAGGGTGTGGGGAATCTGCCTTCAGAGTTCCTCTGAGAGATGCTGGCTGGACACTTTGTTCTTACACGTAGACCTCGCCC
AAGGGTTGGACAGCATGGCTTCCCTTAGTGCCGTGTGACAATGACCAAAAGAGACTTGATAAGCTCATTTCACATTGACTGTCCATC
CCATGTTCAGTGTTCTGCTGGCCACACACTTGGACTGAGTATGTCATCTTGGCATCGGGAAGTGGGGTGTTACTACCATACATCATC
ATTATCAGTGACCAGCAGTGCCTCTTGTGAAGGTTGCCAGCCACAGTGTACAAATTATAATTTTGCAATGGCAACTTAGCTTTGTTC
CTAGCGTCCCCCCCTCTTCCTCCCAAAGTTTTGCAAACAGTAAAAGTCCACCAGGAGATCACAGTGATGGGAACACAGTGATAAGTG
GTAGGGGCCTCCAAGGAACTATTGACTGCTGTCCTCTGCTTGAGAACTGAGAAGCCTCTGATTGCCTTTGAAGTCTGTGGTGTACAA
AGAGTGCGGCCTAGGAAAGCGGAGAGTAGCGGCAGTCCAGTGAATGAGCGGATCTGGGTCCACCTTGGTTTTGCCTTCATCCGTCCT
TTGAGGGACCCCACACTCTGCCTCAGAGATGGCAGGGGGTGTTTGGGATTCAGTCCAGTGGAAACTTGTGAGAGGCTCATGACTCCT
TAACCTCTTCCTTTCTTGGATGGAGAGAGACTTTCTTTGTGTTTGTTTGTTTGTTTGGTTTGAGTTCTTTATATATGGTAGACACCA
ACCCTCTGTTCGAGCTGTAGCTGGTAAAGATTTTTATTTGCTCTCAGCACGCCGCCTCTTCGCCATCTATCACGATGGCATCCTTTG
CTGTCTAGAAGTTTTTTAGTATCGTGAGGTCCCATTTGTCAGTTGTTGACCTTAATGCCTATGCTATTGGGATCTTCTTGAGAAAGC
CCCTTCTTATGCCCTTCTTGTGATCCTCCTGCCTCTGCCTCCTTCAGCAAATCCTACCGGCGTGCGCCACCACTACCGACTCTTATG
CCCTTCTTATGCCTCTAAGCTAAAGACTATTTTCCACCTTCTCCTGGAGACTCAGGGTACTCAGTTTTACTTTGAGGTCCTTGATCC
GTTCAGAGCTGAACATTGGGCAGGTTGGTATTAGGAGCTAGCCTCATTCTTCCATGTGTAGCTATTCCGTTTGATCAGCACCATTGG
GTAAAGATGCTGTGTTTTTTCCCAAATTATATTTTTTGGCCAAGAAAACAAACAAACAAATAAACAAAAAACAATTACAAATTTGGG
CAAGAAGAAATACAAATTGCCGAGAAATATTCTTAAAGCATTCAGTGTTCTTAGCTATTAAAGAAATGTAAATTACAACTGGTGAGA
TTTCCTCTTAGTCAGAATGGCCAGAATCACATGTATGTAGAAAACAAACCCTGGAGCGGATGCGAAGCGCAGGAAGCATTTAACCGT
TGCTGATAGCAGTGCAAACTGGTTTGACCAATATGGAAATGAGTGCAGAGGTTCCTAAAGAAATTAGAAACAGATCTATCGTGTGAG
CCAACTGTAAACTCCTGGGCATATCCCCAAGGACTCTGTATCCTACCACAGAGATACAGATTCATCCATTCATTGCTGTTCTACTCA
CAGTAGCCTGAAAATGAAAACAGCTTAGCTGTATAGTACCGATGGGTAGGGAAAATGTGGTACATAGCCACAATGGAATATTATATA
CCTGCTAAGGGGAATGAAATAAAGACAGGTAAGTGGACGAAGCTAGAAATGAACATTCTAAGAAAGATACCAGCCCCGGAATGTTCT
TTCTTACGGGTGAATGCTGACATTGAATCCTAAGATTCAATGTGTGTGCTTACACTGGAATACATAGAGGAATCATAAAACTAGTAA
GAGACGAAGAAGATTTCAGGGGAAGGGAGATAAATGCAGGAACATGAATGGGGACAGGGGAATAATGAAGCAGGAGGGGTTAAAATT
GGGTGGTGAATGGGAAGGCCAGGGTACGGGGCGTGGGGTGTGTGTGTGTGTGTGTGTGTGTGTGTGTGTGTGTGAAGGAAGCCATCA
TCCATGACTTAACCCTAACCCTGAGTACAACAAACCTTTGAAAAAGCACATGGAGACCTGCTTTTGCAGTTGCCTCTTGTTGGCCAG
TAGGAGTCCTGTAGGTGTACCCAAACATTATTGTGCTCTTATCTTTGGTCATCCTCCAGGACTTGATGGTTAGAGTCTATTGCAGAA
GGCATCATACAAGTGAGTCATGTATGGAGAAATCAGGCTGGTACCCATTTGGAAACATCACGCCTACTAGTTACCTTTTATACTGCT
GGAAGATTCTGTACATACTACTGGGGAAGAAAATAATTAACCATGTTGCCCAGCTATGAACCCTGTGAGCTACATAGTAATAACTAG
CCTGGCAAGATATTCTCACTGATGCAGTAGGGATACCAGTGTTAGGGGTATAACCAACCACTTTCTGCTTTGATTAAAAGCCTGCTG
CACAAGATGGAACCCATACCTTGTATCATTAATTTGGCCAAAAATCTGTGGCCTGCTGGGTCATAGTCTCTAGGGAAAAAGCGTAAA
ACTGTTTATTCTGCTAAAAGGACATAGTTTTAAACTTCCACCTAACCCCTTATCTTTATAAGCATACATCAGTGCATCTCTCAACCT
ACATCATAGAAGTTTCTTTGCAGTGCATAGGGATTAATGCAGAGACCAACAGCTGGTCGGTGCAGAGAATAAGACTATAAAGTGTTA
AACATTCAATGGGAAATTTATATCATACTTAGTGTTCCCAAGGCTCAGGAGTCATCTTGGGAAATGGATTTATTTTATAAGAGCAGT
ATTACTGGACATCTGTAGTGTAATAGTATTTACCAGACATGACAGGTCCTCTACACACATGATCACACAGCAGCTGTGACTGCATGC
ACCAGACTTGCAGAAGATGAAGCCAGCCAATATCCCAACATAGATGGGAGATGGGCTCATTAAGTCCCATCCACTGGCTGTGGATGA
CAATTGGCAGTGTGACTGCAGGTGGGAAGGAGAGTCATTTCTCTTCATGGATCTGACACCATGCTCCAGTAGATGTGCCCACTGCTG
TGCACCAACAGGTGTCACTCTGTGGATGGGGTGAGTTCACAGCATGTGGAGTTGGGAGGGGGAAATAGTGGAGGGGTGGGGAAAGTC
GCAGATGTGAGGATAAAGTTGGATTTGCTCAAAACACTTCATTTTCATCTATTTCAAGTTTCAAACAACGAAAAAAATAAATTAGAG
GGGATAATACGCAGAACTTTGGTGTAGTGTTTGCTTAGCATGTGCAGAGTCCTGGATAGGTTCCCTAACGCTTCACCTACACCTACA
CATATACACGATATAGCTTTCTAAAATGCATTCCAAATTTGTATCTATTTTAGTATTTTTTTTATTACACTACACGTAAGTATAGTG
CCCATGGAGGCCAGATTCTCTGGACTGGAATTACAGGAGGCTGTGAGCCACCTAATGTGGGTTCTGGAAACCCAACTATGGATGCCT
GGCAGAGCAGCAAATGCTCTTTCTGCTGAGCTCCCTCTCCAACCCCAACCCCAACCCCATTCCCAATTTTAGGATAATTCTTTGAGG
GAAAATATTTATGTACTTCAAAAAAAAGAAAGTATTGAAAGTAGAACTTCCCCCTCCCCCTTTGTGAAGAGAGAAACATCTTGCTTT
TGCAAATGCTGTGTCCTGGGACAGGGTGGGAAGTCACAGTGACTATTCTACAGCCCCCTCCCTCGACTCTTAAGTCGTAGACAGTAG
AGGAAACTTACGGGTGAACAGTGACTGATGTCATTTACAGGTAGACGATGTTTTTTGTCAAACATCCTCCGAGCTTTTAAAAACAAA
CATATACTTTGGTGAATAATGGTAATAAAAAGGAAATTGTAATATGAAATAAATTTGAATGAATGCTCCTTTAGCTCATACAGCCAG
GAAATTTATCAGTAGCAATCGGTATGAGACAAATAGCAAAATGGAATAAATTTGAATAAATTCCTGCCCAGGCAGCCATAGAACATG
TTGATAAATATTTCAGCCTAAACTGATACAAAGTATATCATAAAATATTAACTTTGAAGGAATTTCTGTGTTTTGCATTCAATAATA
TTTCGCCAAATAGCACACACATATCAGGCAATGGGGGTAATAAAACTAATTTTCACTAGGTGGCTCACCCTAGTTGATGATCCTGTC
TCTGCCATTGCCTGTGAGTCACTCACCAAGTAGATTCCAGACATCCTGACCCTGTCCTCTGCGCCTGCCTGTATCATGAACTCTCTT
TCAGACTAAGGAGGCAGCGTGATAAACTAGTGTCTGAGTGGCCTCTTGTTTTTTGGCCCTGCCTCTCTCAGCCAGAGAGACCCAAAG
ATTTCTCCAGCTCTAAAAAGATCCACTAATGATGGAGATAGAAGAACTGACAGGGGGACCAAAGAGGTACTGGTCCCTGTCCCTGGC
AGAGTGTTCAAGTCACTTACGTGTTTTCAGGGTGGAACCACTTTATAAGCATCCATTACAAAACCTTGTCTTTGAGGACTGGCTCCC
ATAGTCTGTTTCCCATGCTGATCTGGATGCTTCTCTTTGCTCATGAGTGGGGACCTGGAGTCTCTTGCTGGGGAGGTAGAAGAGGGT
GCCACGACATGACAAAAGGTAGCATCTCGGATGGTGGTCTTCCTATCTCGGAGCACCGGACAGTGCCTCTCATAAGGACCACATCTT
CCAGCCCAGGGCACCGGAGGTACCCACAGAGGCCAGAAGATGATGTTGGGTTTTCTAAAGCTGGAACTGTGAGCCACATATCGTAGG
TACTTGGAAACGATCTCTGGACCGCTGGAGACCAACAAGTTTTTTAACAGCCACCCCTAAGCCCCATGTTCACGGATCTTCTCTAAG
CTAGAGAGGTTGAAGAGTCAGGTGATTTGGAGCTGGGTAGAATATAGGTTCTCTACTCACTGGATAGGCAGCTGGGATGGCACCTCA
TAAGCTCGCCGTCCCTAATCGGACAGTGGATGGAGTTTACCTCACTTTCAGGAAATATATTTTTCTTTTCTTCTGTGGCACTGGGGA
CCGAACCTGTAGCCTCATGCATGACAGGCAGGTACTCTGCCATTGAGCTATAATCTGGTTCTTTTACTTTTACCTTTTCTCAAGGCT
GGGATTTGAACTTGGGACCTCATGCTTTCTAGGCAAGTGCTCTACCACTGAGCTACAGCCATAGACCTGTGTTTGAGACCAGTGAGA
TGATCAAGCCAGATGTTTAAGTAGACCAGGTCATGATAAATAAGACTAGTGCTATCTTCTTTCTTTTCCCCTCCTTCTCCCCTCCCC
CTCCCCCCTCCCCCTCTCCTCCCTTCATCCTTTCCTTACTATCTTTTTCATTTTTATTTCTAGGGAGTATATTGTTTTTGAACTACA
TGACTGCTTCCTGCAGTTGTTTTGCCTTTTAGAAATAGCTGTGATTGGTTTCAAATTTGAATCTGACCCTCTACTTTGAATGAGACA
GTCTCCAATTTCTTTGACCTTTGAGGGTGGTGGGTACCTGTGTTAATTGAACATATGGGGTTCTCTAGTAGACAGGTAAAAATACTC
GTGTCTTTGGGATCTGGGGCCATGCTCATTATAAGTAAATATTTGGGTACAGGGCAGCTCCGTATCTGTGTGTGTGCTCTCAGTGTG
TGTGTGTGTGTGTTTGTGTAGGGGGGGGTGTAACATTGTAGCATGCTACACCAGTTAAGAATTGCAGTGGTCAGGAGGCTTCTATGA
TTTGGAACTTGGGTGGTAGAGTCAGGCTTTTGGGTCTGGGTCTTGCCGCTGGACTTTACCACTGTGTGACCTACCCCCCCCCATGAC
TGCTGCCAAGACGGAGAGAACTGAACGTTGATTTGCAGCATGCTCACTCTTAAGTGGGTAGCATGCTTCCGTTTCCAGCAGAATGGC
GGACACCCTTGTAGGTAGGCATCGCAGAACTGACCATTAGTCGCCATGCATCTGGTACATAGACGAGGAGAAAGCTTCTTTCTCGTT
CAGACTGGATTACACCCACATCAGCCTGAATCTCACTACCGCTGCCATGCTCAGTCTTCAGATGAATCCAAGACTTGCCCTGTGACT
CCTTGTTTTAATTAGCTGGCTTGGGTTAAGTACAAGGGCTAATTGCTCCAGTCTGGCCCCTGGGTTTGGTTGTTTTGTTCAATTTAC
TTATGTAAGCAGGCGTTCTCTATGCAAACATTTGTGGAAGTCACAATCCCAAAACAGAGTAAATACAGAAGCAGGCTGCACACTGCA
GCAGTGTTTTAGCGACAGTGAGAAAACATCATCTGTTCCTTCTCCTACTTGGGGCTAGCGTGAGCTCTGTTGGTTTGCTAATGTTCT
TTGCAGTTGATTGATGGATTCATCGGGGATTTTGTACCACTCCCTGCATCTGTGCATTTAAGGGCTATTTTATTGACATGTCCACAT
CGTGGCTCTCTTTCTTCTTTGTAAAGAGGTGCCCTGGGCTGGATAGCCGAGAATGTGGGGGATAGGAGAAACAGCTGCTTTTCTGGT
TGGTGGCAGAACTGGTACCCAGCTGGTGTCCTGGCTCCTCTGGCAGTTGGGTATGTGGAAGTGCTGCTTTGACTCTTTAAAGTGTTG
TTTTTGGTAGAAACAGGGGAAAACTGATTCCCTTTTGTTGCTCAGCAGCCTTACCTCCCTCATGCTATTCATCAGAAATAATGACTT
CCAATTTTTTCTCCTTTAAATTAGATACACCATTGTTCCATCATCTATCAATATTTCCTTTACATTTTTTTTTCTGTTGAGAGTGTT
GGCATGTTCCCAGCTATCCTGAGTGCTGGTGTCTGGATTATCAATAGCCCTGGCTTTTCCAGGCTCTGTGTGGCTTACACCCTGACT
CCTCAGGTGTTCCTGGGGTGGGCAGGTTCAGACAGGATGCCCGAGGGTGAGAACAGAGGCTGCACTGAGAGAATCTGGGGAAGGCAG
TGGGTTCCCTCTTTCATCAGGATTTCTGCTTCAGGCTGCCTTTTGCATTTTCACCTATTTTTATATTCTCTTCAAAGCAGTTTTGAA
GTGATAACAGTCATTAACTTCTTTCCTGGCAAATGAACTGAGAGGGGATTTAATGCAGCCAGTGAGAAATGAGAACCTCCCTTCAAA
CCTTTCTGGGGGAGTTGCTAAGAAAGAACAGTTGAACTCCCAAGTTTTTATGTAATTTTGGGGGCGGGGGCTGGGATTTGGAAAGTA
TTAAATTGATCTCATATTCTGAAATTGCGTGATCAAAAGAAACTCAAGAAGAGCAGTCATGTGACGCCGTCAGGTAGCGTTGTCTAC
TTGCATTAGTCCTTTGAAGGTGCAAGATTAAAATTCCTGAAGCCCCAGCTCTGATTACTCCCTAATTTTGTCCTTTTGGCTTTTCTG
AGCTTTGGATTCAGAGACATGGCCTCATTTTGTGGGCTGGTTGTTGGATCAGTCTCGCTGTCGTGGATGTAGTCAGTACTACCTTGA
AGGATGGAGCTTGCAATTAAGCAGAAGCAGCCCAGGGGTGAACCGCTAAGCACTGCAGGTGTCAATGAAGCATAGGGTGGAAGAGAT
GAAAAGCTAAAAGCCCCCCCATGACCTCTTTGCACACCGATTATCCAATCTGAAGTCATTGGTGCCATTTTGCACATAAGGGCCGGG
ATTGCCCTTATGACCACGTCCTTTTCTAGCTAGCTAGGTAACTTCCCTTTTCATTTTTCAAGCCAAGGCCCCTCTGTGCTTCATTGT
AACAGTGAATGGGGAAACATCTTGAAGTAGTCCTTAATCTCCCCACACTCAGTAACATTGTATTCCATTTCATTAAGGATGAAAGCG
CTGGTCCTGACCTATTAAAGGGACAGCAGGGGGGTTGTGACCTGCTATTTGAAAAAGAGTGGTCCAGAGGCTCTCCATCCTCGGTTC
TCAGCATCCTCTGCCCTCCGTGGAGACTTGGCCCCTTCATGAAACCCTCCCTCCTCTCAGCATCTGGTGCCTCCTCTGACCTTATCA
CTTTTCTCAATCTCATTTACAGCATTTACTGCAAATTGGCTCTTATGCTAATTACTGATTTACAACTTATCTCAGTGGCTTGATGGT
GAGTGCCTGGTCAGGGCATGAGCTCCGTGCTGACCTCATCCATCAACACACACACACACACACACACACACACGCGCGCGCGCGCCC
CAGAGTGTCCTGGCCACCTGAGCCCTTGCAAAGTGTGCAGTGCTTGGATGCGCCTCTAGCTCAGTGCTGACACTTGAAAGCCAGGAC
ATCACACTGCCACTGTTACAGCTCCAGGGGCATGGCTATCAGCTGGGCTGCCTTGATTTGTTACATATCTGAAAAAGAATGAGATTA
GGTTGGGGTTGCAGGTCTTTAAACCTTTGACCTTTGCAGTGTAACAGGTAATTACTTCAGCCTACAAGGCCTGCCTTTTACAGCATG
AAGTGACTTTTCATGTGTTTTGTGATGATTTGTGGATCCTTGACGGGATGGTATGCAGATGGGGCGTTTTACTTATTTATATTGGAG
CCATTTGTCTACAATGTGGAAACCTCACACAGAGGAGAAGGAATACAGATACCTTAATCATACGCCGTCTCTCACATCTGGAGCTCA
GTTTTGTGCTTCTAATGATGGCGTTCAGAGAAGAGCAGGACCCATTTTACGTTGACACAAATTATCTACCCAAAGGCTGACCTTATT
TAAAGGAAGTGATTCAATTTAGAAAGGTTGTCCCCATTCAATCTAATTTCAGAAACTTGACTGTTAAAACCTGTCCCTATACCATCT
TGACTAACAGATGTAGCCATAGCCCCATCCATCTTGGGGAAGTGCCTGATTAAAATATATACCATCCCTGGACTTCCCAGACTCTAT
ACATTTGAGAAAGAAATGTATGCTGTTTATAACGTTCTTAAAAACGGTCTGCTAGAAATACCTAGGAAGTCACTGCTGAAGAAAGAG
CTAGTCAGTGATGCGTACTTTTAGGGGCTGGGCAGTTTGCATTATTCACAGTGGATTTCAGTAGGACATTTTCATACATGAGTGTTT
CTTCATGTTCACTCTCTCTCTCCTACCCCAGCTACTCCTCTGAACTCTTCCCAACTGCCCCCTCCCAATAACTTTCATCTCTCTCCC
TTTCCTGGCAATCATCAACTATGCACAAACGGTTCGTGCTTCTTATAATGGCTTCTTTTCTGGGCTTGGTGGACATCTCAGCATATT
TAGCAAATCTGCTCATACATAGGCAGATTTCGCTTTTGAGCATCAAACCCTCACGGAGACTTTGAACTGGGTTTAGTTTAATTCTGC
ACTCAGCGAATTTGCAGACAACTTGCCTTATTTGGACTCCTTCCCACCCCGCCTGCCACTTACCCAAGCTAAAATGTATGTCAAAGG
AAGCGGGGCTCATCTTAAAGGAGATTTGGAGAAAATCTTCCTTCAATATTTAAAGTGCTTGACAAAGTCATGTTCACATGGAAGTTG
GTTTTAAGATCAGGAAGTTAAAATCGGGCGGCTTATTTACATTACTGTCAGAAAGCTGTTCTAATAGGATTGACATGGGGCACTAAT
TAATCACCCAGGGTCTCTCCATTCACTAATCATATTGCGTGAAACTTCCCTGCTGGCCGAGGCTCAAGGGCAAACCTTGGGCTACCC
GCCAGCTTATTTTTCATAGGCATTATGCTACTGCTATGAATTAATGCAAGCCTAATGTATTCTGTCAGTGGTTTATTCTCCCATGGC
GATCTTCTCTGGTTAAACAGCTTTAAATGAAATTTTATATTAAGTATGAAAAGAACTTTTCTTTCAGATGAGGGAAAGGGGATTAAA
AAAATCATCTTTGTAATCTTTTTCCCCCACAGTCTATACTCTTTCAGTTCTGGTAAAAATCTGCTAGATTAATTGAATAAGGCACAG
ATGACATGTTAGTTTCTTGCCATTACCTGAGAAGTGACCTTATATTAAGCCTAGGAAACGTTCCCTATTACATTTTAATTGCAGCAG
CAATGTGTGCTTATTAAGTATCTACTGCACACAAAACGTCTCAGCCACTTCTTCTTATGTCTGCCTTTGCAAATACAGTAACTGTAT
CAATGCGAACCCTAGTCTGTTAGGCTGCCTAGGGTCACATTTGGTAAATGCTGGAGCCAGGTTTGAACCAGAGCTTGTCCATTCAAG
TTCTCCCTTATCGGGCCATGTTACTGTTTATCATTTGTACAGTTCCCCCATACTGTCATTTTACCTTGGGACGGTCCAGTGGTCTTA
ATCTTCAATAATGAAATGTCTATTTCCACATTGACTCATTACATTCATCTGAGTCATTTCTGAGGCATGTTGGTTTGGGGTACATAA
TTTCATCTCTCACTTCACCCTCACGGTGCCTTATTCAGAGAACAAGAAGTTAGCGGCATCTTTGAACCAGAAATAAACATAGCTTAT
TCATCTCCCTCATGCTGTTCTTCATCGCTCTCAAGAAGAAGAAAAAAATCCTAATAGAAAAGGAAAATTCCAGAGTGTATTCACATG
GAGCGCTCCCAAGGCAGTGCCTGCCGAGAGCTCAGCTTCCCATCTTCAAGCTGCCTGTGCAATGGCGACAGCTTGTCCTGTATGCAA
ATGAATTCCCAAGAGGCTGGGAAATAAACTCTTACATAAAAGAACATATAAATGGCTGACGTGTCAAAACCCATTCCTTAAGAGAGC
AGGACGGTACTTGGTGGCACAAGCGGGGTTTCCCCGTGAAGCTCGCTCTCCTTAGTATGCTGAGCTGATGTTCTTGGCTGAAAGAGA
TCCATCCCGTTTCTTTTTATAGTGAGCGTCATGAAAAGTTGGAAAGGGGGAAAAACTGTGCTGAACATACAGAATAAATAAGTAACT
GCTGCAGGCCAGGCAGGCTGCCTTTTGTTTTCAGACATCCACTCCTTCCCCCTTTGTCCATCACGGATGCCTGGTGTATAGAGCGCT
TCTTATGGAGGTTGTTTGTCATCCACAACAAAGGGCTAATTGCGTCTGGGCCTCCTTTCACATGATAATCAATGGATCTAGATCTCC
TCTCCCTATCTTATGTTCATCTCGTCCTTATTTATTTATTTTAGGTTTTGATTGAATGTAAAGGATCAGAGAGATGACGCCCACCTA
GTAAACGCATGAAGGGAATTTTAAACGTGGGCACGGAAAGGAAGGCACCTGCTACCACTTTTCTTGTGATTGATTTGAACAGAATTA
TAACTTAGAATAGCTGTTGAAAAGACTCCGTGGTGTTCCTAGGCACTGATGCTCTTCTCTGAGGACCATTTCAAAAAGGCCCTCTGT
GAGGAAACTCAGTCCCTGTTAGAAGAGGGACCCAAGGTGCTTTGAGGCTACTTGGGGTGCTCTATGTACATTCATTTCTGAAGTGTT
CTAATTTTATTCTAATTCTAATGAGAAGTGAGTCTGTGTTCAACATGGGTTTCTTTTTGTAGCTTTGCCAGAACATACCATTGTGTA
TGTGTGAGGCATGTATGTGGCTTTACGTTTGAGTGTCAGAGTAAGCTAACTAGATGAAAATCTCTCCATCTACGGAGCTTATGCTAC
CATAAGTGCCCCCATTTTGTGTATTTGGAAACTAAGGAACAGGAGGTTGAATTACTTGACCAGGCTCACATGTTTGTGACTCAGACC
TAACTCAAGATAGAAAGACAGGGAAATATTTGGAGATCCCGACAAGGGAAAACAACACTTCTCCACCAGGAGTCTAGTTGATGCCAG
CCACCCAGCGGCATCAAAGCAAGAATTTCAGAGCTATTAATAACTGCCTAAGGCCTTCCTCCCTTCTCAGCTATAAACCTACAGTTT
GCTAGGAATTTCCCCAGGAACCCCGTTTACAGGGAAAGGACGGATACTAACTTACTACCCTTGTGTAACCCTAGTAACTTCTGGGTT
ATCATATTATAACCCAGCCAAGGCATCTCCAGAGCCTCCCTATAATCTCACATGTCAAAAGAATGAAGGGAGAAGAAGGCATCTTCT
TAGCAGTCTTTTGAGATGTAATTTGAGAGCCACCAAAATGGTATATATGGCTGATATCAAGACAATTGTGACTTTCCCTTGAAAAGC
TCAGCACACACACACACACACACACACACACACACACACATATATTTTTCTTCCTTTGTGACTCTTAATGAATCCCAGTCATGCACT
TGGGTCTGTCTTATTTTCCTGTGAGCTCCCCCCTTTCTTTTAATCTTTATTTTAGAGCCCATTTTAAAATAATCCAATTTTACTTTA
TGAACTGATAGTCCTGAAATTCTTTTCTGCCTTGAAGCCAAACTTGAGGCCCATGGAAAGAGTCTCCAGACCATGAGTATGGTGACG
TGGAACTCTGTCTCAATCCCCTTCACCACCGACAAACCTAGGCCCATTTACCTAAGGGTCCATAGCTGTCACCGTTCACCCTTCTGC
TTGTTGTCACAAAGTCCTCTGGCCTTATGGCTTCTCATAGTCTTCGCGCAGATTTTCCTGCTCCTTCCCGCTTTGGCTGGTGTCTTT
CTCACCAGGATGGCTGCAATGGCCTCCCAGCTAGCCTAACTACTAACAGCTTGGCCTCTTTCCCTTTGTTCTCTGTACTGTAGTGAA
TAAGTTGTCCAAGCTGCAAACCTAGCCCCCCCCCCCCCCCCGTCCTGCTTGGAGCTCTAACTTGGTCTCCCATGGCTTGGTGGTCCC
TGGGTGTACACACCACACATGGTGTGCTAACCATGCCCTAGCtTAACCCTCTGGCCTCTAGGTTCTCAAGCTTAAAACATCACCTGC
CTGGGATTGAAGAGATGGCTCAGCTGTTAACAGCACCTCTTGTTCTTACAGAAGACTCAGGTTTGCTTTCCAGTATCTTCCTAGTAC
CTTGTAACCCAGTTCTAGGTGCCTCCTTCTGTCCTTTCTGGACACAAGGCACACATATGACACGCTCGTACCTGTAGGCAAAGCACT
CATTCACATAAAATAAAAACCAACCTTTTTCTTTAAAAAAGCCACGCATCTCGGTGACTGTGCAGGCAGGTTACGGAGAGAGCTGGC
TGCCTTCAGCCAGTTTTCATGGGGCGGGATGAGGGATGCCTGAGATGGTGCATTTCTGATAAGTCCCCTGGTGCAGAGCCATCCTGT
GAGGACCACTGCTGGCCTGAGCCTTGTCGCACGCACGCATACTTGCAGACCCCTCAAGGGACCCACCATGCCACACTTTGATCCTTC
TGCATCTACTGTTGCCTGTGCAGGAGTGAGGCTCCCTTTGTCTCCCCTTTCAAACACAGCTAGAATGTTGCTTCCTAGGAAGTTTCC
TCTGTCAGCACCCCCACCCCAATGCCTCTGTTTCTATGGCAACTTGTCTATGCCTCCAGGAAGACTCCATTCCCTGCACCTAAGTTG
TGATTTTATTTATGTGCCCCCATTAGCTCTCTAATAAAAGACATCATCTGTTGTTGTTTTTTTCCCTTTTGGGGGAGGGGCGGGCAA
GGAGGATGGCATTCAAGACAGGGTTTCCCTGTATAGCCTTGGCTGTTCTGGAACTGGCATTGTAGACCAGGTTGGCCTGGAACTCAG
AGATTCATCTGCCCCTGCCTCTAAGTGCTGGAATTAAAGGTGTGTGCCACCCCTGCCCGGCTTTCTCATTCTCCTTTCTATCCCTGG
CACCTAGCTCCGTGCCTGCTCAGTCCTGAACATATGAAACATGTTGTATGATTTAATCCTTCTTCCACCATTACAAAGAAAAATCCT
ATTTTGAATTGTATTTGTGTTAACTATTTGTTTGTTTGTTTTAGCTCTTAAAACATGTCTGTGGTATTTTGCTTTTAAGTTAACAAG
ATCTTTGAGTACAAAAGCTGTGCATCTAAGAGTACTAAACTTCTAGACCCAGTTGGTGAGTGCTCTGCATCTTGCTGCTTAACTCAA
ACAGTGAGAGCCTCTTCCCACAGAGGTTCATGCTTGCCTACAGATAACTGACTTTTTACGCCTGTAGTCTGACATACTTCCAGTCCA
TTTCAGCCTTCAGTGCGCCCCTCTGGTGTGTTTTCAGCCTTGGCTTTGTCTAATGGCTGGGGGAGGGGTAAGCAATAGAGCAGCCCA
TCAGGGCAAGCCTTTTTTAAATTTTATTTATTTATTTTTTTAACTGTAAGACAAGCAAAGATTAGTGAGCTGTAAGCTTGCAACTTC
CACGTTTCTATCCAGAAGTGGATAGCTAATCCCTCATGTTCCTTTTTTATATGCTTGCATTGCCTGGGTAATTTCCAAGAATATGGA
TTTTATTGCTTGCGTAGCCGTCCCCCTCCCCATCTCAGATTACAAGCTCTTGCTCAAAATGTCAAGGTCCAAGTTCTAACAAAGAGC
TCTCTAGACCAGACTGCCTCAGACTGTGGGCATTTCTTTAAGGCCTTCTCCTGGAACAGTGTGCATTCAAAGGGAAAAGCCCTTTCA
TCGTCTCTGGGAAGATACTGCGGGCTCTTGGTGCTACGCAGTTCCAAAGAAGTCCCCCTCCTGGCCCCTTTTTCCTTTCCCAGGCTC
TGTGTGATGGACTGCTCCAGATCCTGTTCTTGGCTTCGAGCCCCAGCAGTCTGTGTCCGCAATCCCATAAACAAGCAGAGGACTAGC
CCTTCAGGTGGATGGTGGAGACTGAATTAAGTATTCTTGCCTCTCGTGCCCGGTCACAACAGCTGGCCCAACAGTTTAGAACATCTG
GGGTTTTCATTGAGCTGCCCTGAAAAGAGATCACAGCTTCGTACAAGTCAGAGGAAGCCCCCAGCACTGTGGTTGTAGGCTTCTCTG
AATGTTTTGCTCATGCAGCATGAGAATTATGTGGATCCCCTAAAACTGTGGGTGTAGGTATTTTATAGCCATTCATGTAAAGGAGCA
GAATAGATCTCAAACTAGAGTCCGTTAGTGTCTCTTAAATATATAACTATTTGGAGCACAGTTCTGGAAGGATGCTCAAGCCGGTGA
CAGAAGAAGGAGAATTAAACAGCACTCAGGGGCCGTCCGTCACCTTTGCTTTATCCATGCACGTGCTCAGTGATTAGTATAGGCCCT
GACTGTCCTACTGTCTGACGTGAGGTGTTGTCATTCTTTCCAGTCCTGTGTACACATGTAGGCAGGACAAGGCCATACCTGGTATGG
CAGGGAAGCTCTGAGGACAAGGGGGCCTGAGTTCATCTTCTACCAGGGCTTTTCTGACTCTGAAAGCGGAGCTGCCTGTTAAGGTGG
CCACTGTGGGAGGAGTTGGGGAGCAGGCAGGGTAATGTCTGAGGAGCGATGGGGGGCAGGGAATAATCTGCACAGACTCCTTGCTTA
CAGTTGGCTTTCATGTGGTTTCGTGGACCCCACAGTTTGCAGCAGTTAGTAACATCAGTCATTGGTTAGATCAGGCAAGGCCTGCAG
GCTCTGGGAAGCCAAAGAACCCTTCAGGGATTCCTGCCCCATCTGAGGACACAGTGTAGTCTTCTCAGTGGCCTGTGCAGCCTGACA
CAGCCTGGCTACTGTGAGCCTCTGAGCTGTGCTTCTCCGTTCCTGGCACTTTGTGTCCCGCTTTGTCGCCATCTGTCCGGGGCCTCA
GTCCAGATGTCTGGCTTCGGCTAGGGTGCTGTTCGGACATCCGCCAGTCAGCTCTTCCTTCCTTCATGGTGCCCAATGAACATGTAT
TTTTTTCCCCTTAATATTTATTTTGTTTTTATTTATGTGTCTCTCTGTGTGTATGCTCCAAAGAGGATGTTTAATCCCCTCAACTAG
TGGGGCTTGAACTACTGGTTGTGAGCTGCCCAACAGGGGGTACTCAGACCCCATGTCTAGTCCTCTGTAACAGCAGCAAACTCTTAA
CTAATGGAGTCATCAGATACTAATCCCTGTCCCTCTCGTGGGTTTGCTCTAGTCACTCTGCAGTACCTGCAGTTGCCTTCCACTTGG
CTTTCTCTCCTCACTCTGCCCTCCTCCCTGGCAGCAGCCCTGAGCAGCCCGCTCATCTGTGACTTTCTATTGCTTGTCTTTCCTTGC
GCTGCCCTGCTAGCGTGTCAGTGCTACAAAGCAGGCACTTGTCCGCTCAGTTCCCTGACTTGTCCGAAGCCCTCCAGTGGCTGCATT
GCCGGTTGTAGTTGCCCAGCAGGCTCTGCCTTTCCCGAGTTGTATTATGGTACTGTGCAGCACCAGGGCTAGAGGTCCCTAGGCAAT
ATGTACTGGGGCCACAATTAGATGGCCTTGTGACTATGTAAAGGGAGAGACATTGGCCCTCGCCTCCATGGGAATTGTCCTGTTTAC
AGACTCGAGTTTTGTCTGTCATGCCATCTTATCAAGACCATTCTGGACAGTTGTAAACTGGGACATCAGAGCCCAGTGGCAAGAGTA
GGATTGATTCTCTGTTGGTTGAATTTTCTGCCAGCTCATACTTTGGCAGGAATTGTACTCAGGGACTTTTTTTCTCCTGAAATAACT
CCCCACCCTACCAAGACAGGTGTTGTATTAGTGGCTGTACATAGGATACCTGTATAAGCTGGCTACCCATCTGCCAGAGCTGCAGTG
TTGGCTCAGAAAAAAGGAGAGTGAGAACAAGTGAGCTTCTCTGCTGGCTCATTGGTCTGTCCAGGGAAGGCGGTAGGTGCGCCTCGC
CTGCCTGTTCCAGCGGAGGTTTACATTCTCAGAACGTTAGTCCGCTGTTGGTGAGGCTGTTGGTGAGGCTCGGCTTCCTGCAGTGCT
GGTCGGGAACTTTGTTTCAGTCTGGACAGATCGCAGGTGAAATTGCTACATCTTAAGCAAACAGGAGTAAGGAGTGCTCGTTTAGAA
GCCGCTTAAGAACTTGGATGGCTGGGCCCTGGTATGTAATTGCTTCTTTTTAGGAATACAAGTAGCAGAGGTGACAGAAATATATGA
TGATGGTAAAATAATTTAGTTTTCAAAAATACAAAACGTGAGATTTGCTTCCCAGAGAATTCAGTTAGCAGCCAAATACTCAAACAA
GAAAAACATCCTTCAGAATTTCTGCTAGGTCCAGCCAGCCCTGAGAACAGCACCCAGAAGTCGATTCTGTCTGCAGAGCAGTAGGTT
TTATTTCCCCTGTACTGGGAAGTTGAAGACGGGGCCCAGTGTGTGCTTGGAAAGCACTAACCACTGAGCAGCAACCCAGACCCACAT
TGGAATGTTGAAGGGCCATAGTTTCCAGGTATAAGACCAATTACCCTTCACACCTCCATCCTGTCATTACCGGAAAACACTTCTCAG
TAGTTCTCAGTTCTGCCCACGTTTCCTCCATATCCTGGACACGATCAGCTCCTGCCTGCTCCTCCCTCATCTTCCTTTGGATCCCTG
TGGATCCAGACGTCTGCAACCCACATGGTGATTCTTCTCCTCAGTGCCAGCCTGTCCCACCCACTCCCACCCACCACTCAGTCTCAC
AGATGGTCCCTGAGAATCCTCCTGTCACCAGGGTGATGAATGGGCGCCTTACAAGGTCCAAAAGTGCCGTGCCACACTTGCGTCTCT
GATTGCATGCCAGCCCCTCCCATCTGCTGTTGTTAGCCACACAGATTTGGTGACTTGTAATTCTCTCCTTACCACGGGGCAGTTTTA
TTCTTTCGTGACCTGAAGTCCTCTGAACTGTAAGAGCTATGTCTGTTTTTTCTTATCTCCTTTTATCTTGAGACGTGTAAGGTCAAA
GTATAAAGTTGGGACTCAAAGCGACTTGGAATACTGGAAAGGTGGCTTTTTGAAGTGTCCATTTCGTGCCAGTTGGGATGGGACACT
GTTCCAGTCCAAAACAATGGAGTCTGCCAGGATGAAAATAACTGGATATGGAAGAAGAGCTTTTCCACTTGACAGTGGGGCAGATGC
ATCCTTCTGGAATCACCCTGTGACACCCGAGACTATTTTCCTTTCCAGTTTTGCCAAAGCATCAGTTCTGCTGGCTCTGGAATCCAG
GTCTAGCTTCCGTCACTGTACCACTCTTGGTTCCCATGGGGCCATTCTCAGTTCTGGCAGAAGAATGCACCTTGGATGCCTAGACAT
CAGAGGAGCAACTTAGAGTATTAGGGCAAGCACTTCCCAGTGTTTCCACTGTGAGCTGGTCTATATAGAGAGGGCGTTTTAGACATT
AGGGGACACACAGGATGAGCATGCTCAATCTGAAAAACCATAGGTTTGTAAGTACCGCTCTAATGGCTGAAAGGTTTTGGATTTTCA
TGCTAAAGCTGTTTAACTGTAGTAACCAGCAAAGTCTATGCAAATATACCCCAATTCAGTAAACTCTGAAATATGAAATACTCCGTC
CGGTTCCAAGCATTTTAGTAAGGGTAATATGCAGGGCTGGAGAGATGGCTCAGTGGTTAAGAGGGGCTGGAGAGATGGCTCAGTGGT
TAGGAGCTCTGGCTGCTCTTCCAGAGGACGTGGGTTCAGTTGCCATGTATACATCCATGTGGCAGTATATATCTATAACTCAGTTCC
AGAGGATTGGATGATCTCTTCTGGCTTGTATGGGCACTGCAAGCAAAATGCCCACACACATAAAAATAAGTAAAATATTTTAAAAAA
ATCTTTTAAAAGGGAATATTCAGTAGTACCTATTCCCTGAGTGACACAACTCAAGCACAGGCCTGTGCACTGCTGGGAGAAACATTG
TTTCTAAATGTATTCTGCTATCTGTTAAATAAGAGTGATGTCAAGTATCTATCTACTGCATGGAATTGCTGTGGGATACTTCAGGTG
AACATTATTCTGCACAGATGCAATTTAATGCAGGCTTATATTTTCCAGGAGTTATGCTTTAAAAAAAGATAAAATTAATTATAAGTT
GAAATTTGCCAAATAAAATCTTAACATTTCAAAGCAGAATAATAACGAAGCACCAGCTAATGGAAATGGCCTATATCCTTTTCATTC
CTCCTAGCTCTCGGAACCTGGCTGGTGCGCATGGAACAAGCGCTTGCCTTTGCACCTCAGTATGAGCTTGTTGCCTACCAGGTGATC
ACTGGCTACCACACTGTTAGTACTGGTTTTGGAATGGGGCCAGACATGGTGGCACATGCCTTTAGTTCCAGTACTTGGGAGGTAGAG
GCAGATAAACCTTTGTGAATTTAAGGCCAGCCTAAGTCTGCAAAGCAAGTTCCAGGCCAACCAGGAGATGAGACCTGGTGAGATATG
TCTCAAAACAACAAGTGTCCTTAGTAGGACCTTCTTGCTGGAGATGACTTTGCTTGCAGGACTCAGAAACCAAACTGGAACTGACCT
GGAATCGTCTTTACTCATTAATGCTTGGCTTTCATAGTGACCAAAGTACTAGCTGGGCTGCTGGGAGGAAAAGGCATCCATGAGCTT
ACCCAGTGGTGAGCCCTGCATGCTGCAGTACCAACCTTAGAGGGAAAACGTGCCAGCCAGAAGTCATGGCTTGTCAAAGGCCAGTGG
CTGACATGGTCGCAGGTCCTTGGAGGCAACGTGCTCCTGTTGTTCTGGGAAACGGACATGTTACCAGGTTTTCTTGTAAATATGTGT
TTTTATTCCACTAGGTTAGTGTTACTCTCAGTCTTGATCTTTTTGTATTAAGCGATGGTCAGTAAAAATTCTTATTTTTAGTCAGGC
GGTGGTGGTGCACACCTTTAATTCCAGCACTTGGGAGGCAGAGGCAGGTGGATTTCTGAGTTCCAGGACAGCCAGGGTTATACAGAG
AAACCCTGTCTTGAAAATCAAAAACAAAACAAACAAACAAAAAAAGATTCTTATTTTTTTTGAGAGAATGTTTTATTTATTTTTGAA
TTATTTATTCATTTTATGTATATGAGTACACTGTAGCTGTCTTCAGACACACTAGAAGAGGGCATCAGATCCCATTAGAGATGGTTG
TGAGCCACCACGTGGTTGCTGGAATTGAACTCAGGACCTCTGAAAGAGTAGTCAGTGCTCTTAACCAATGAGCCATCTCTTCAAGTC
CCAGTGGCAGCAGTGGTTATCTGCTTAACACGTACACAAGATCGTGCCAGTCTTATAATTACAGCAGGGGGGAGGGGAGGGGTGGGG
AAGGGGCTCATGATGATCCACCCCTACCTGAGGGCTGTTGGCTTAGGAGGAGTCAGTTTGTCTTTGGGAATGTGGCCCATCCTCAGG
CATACAGCACTAACTGGACTCACTGAGGTAATTGTTAGTGAAAGAAAGAGAGAGGACGTGGACATGAGGGGGACATATGTTTGGGGA
GCCTGTGGGAGAAAGAGGGAGGTTCCTCATGGATACAGATCATTGTATAAATGTATGATAATTTAAAAATTTAGTATCTTGGGGCTG
AGAGTATAGCTTAGTGATAGACACCTGCTTAGCTGTTTAAGAGGCCCAGAACTGGCCCTAGTACCAAAACCAGAAATGAAAATAGCG
ACTTAGCTCATATTTAGCATCCAACTAAAGTATTGTTACTCATTTTACAACTTCAACACTTTGATGGACAGACATTCGTGCCAACAT
TTGCATACACTGCTGTGTGGGAAGAACGTGGGGTTCGGTTTTGATTCTATCTCAGTAGAGTAACTGGTCACCATGACGAGTTCCCAG
CGTTCCTCTGCACCCCTCCCCACTGGGTTTCACAGTGAGGTCACTGTGGGGTTTCTGGGCATCTTTCCTTTGGGAAGGGGAAACTGC
TTCAAGTGCTGCGTGCCTGTCTCTGGGTTTTGGCTCTTCACTGTTAATTATTTGGAGCTGGAACAACTTGATTTTAAAACAAAACAT
ATGGGCTGAGAGCAGTCGGGAATACATTTGCTGCTGATGTAGTGCGGAAGCTATGCAGCAAGCCGAGGTCCCGCCCTGTTCTAGTTA
CTCGCATAGGTATGGAATGGTTCCCGTAAATTTCAAGTCATGCCCCTTTCTCCAAATTCTTAAACTCCCTTTCGGAAAGACACAAGT
CAAAGCAGGAAACACTGAAGCAGATTGTAAAGAAAATTGTGAATCTCTCCTCTCTCCCCCAACCCCTACTTTCCTCTTTCTCCTCTG
TGGAAAACCTTATCTTTTCAATTTTTTAATTCTGATTCTTAAAGTTTTTTTTTTTTTTTTTTCCCCAAAGTGCCAAGAGGAGAAAGG
AGAACTGTGTATATGTAAAGGGAAGAAAGCCCTCTCCACACCCCCAGCCAAATTTCTCACTTTCTCCCTGGTCAACACTAGAACGGA
GAATGTCCCTTTCACGATCAGCACTAAAGCGAAGGCGACATTTTGTCCCTTTCTTGAGATAGTCTCTTGTAGCCAAAGCTGTACTGG
AATTCCAGATCCTCTTGTGTCTTTGGGTTGAGGTTGTAGGTAGGCACCCATCTCTTACTTCACCTTCTCTGCTTGCTGCCGTCAAGC
CATGCCGTGTCCTGGTCATGTCTCACAGAATTCTGGCTCAAATGCCATCCCTTTGCCATCCCGGGTTGGGGGTGGAAATTCATCAGT
GAGGACTCTTGCTTGCCCTTTATCAGATCTCCATTCCTCCTCATAGCACCTGTGAGTTCTACAGAAGAACCTACCCATAGTTATGTC
CCCCTTTGCTAAAAAGGAGGCCATCACAGTGCCAGTTTCAAAGTCAAACTCTACTGTGAGGCTCAGAGAAGACACTCCCTCCCACTC
AGGAAGCAGAGAGCCCGGCAAATATGGAAAGCAGAGTTGCAGGAGCGACTGGCAGGGACAGGCATGCACCCAGCCTGCTCAGCAGCA
GTGAGACAGGGACTTGACCATGATACCATCTGTTCCATCCCATCCACTGCCAACAGTGATACCGTGGCTGAACTCTGTGTCAGGAGC
CGTGACTGTTGTCCACAGAGAGCTTTAGCCTTAGAAGGTCGGACATTTCCCCCTGGGGTGCATTTGATTTTAGTTGCATGCAGCAAA
GCCTGAACTCTGCCCTATAGAGCGTCCCTCATCTGACAGAGTCCTGTGTATTCTCTTCTGTCTGTCAGCTATGACTGTATTTTGTCT
AGAACCTATAAATACAGTACTAATACGCCAGGTTGCTTGGTTCCTGGACCTTTTTCTTGTTTTATATCAGGAACATAGCACACTGAC
CAAGGAAGTAGAAGGGCTGACCATAGTTTTTTCATAGGCATTGAAAACGAGAAATCTTTTTTTTTTTTCCTCCAAGACAGATTTTCT
CTGTATAGCCCTGGCTGTCCTGGAACTCACTCTATAGACCAGACTGGCCTGGCCTCAAACTCAGAAATCCGCCTGCCTCTGTCTCCT
GAGTGCTGGGATTACAGGCTTGCGTCACCACGCCCAGCCCTCTTTTTCTTCTTTTAAAAAGATTTATTTATCTTACACTGTACACTG
TTGCTGTCAGACAGACCAGAAGATGGCATCAGATCCCATTACATATGGTTGTAAGCCACCATGTAGTTGCTGGGAATTGAACTCAGG
ACCTCTGGAAGAGCAGTCAGTGCTCTTAACCTCTGAGCCATCTCTCAGCCCCACAAGAAATCTTTCTAAGCACGTATGCTGGCAGGT
TTTATGTCAGCTTGATATAAGCTAGAGCCATTTGGGACTGGGGACTCTGGGACTCTGTGTCTACACGTTTGGTCTGTGGGCAAACTT
AGGCAGACAAGTCTGGGGAGGCATTTCCTTAATTAATGATTGCTGTGGGAGGGTCCAGCTCACTGTGGGTGGTGCTGCCTCTGGGCA
GGTGATGGTCCTAGGTTGTATAAAGAGGAGCAGACAAAGCATCCCTCCTTGGCCTCTGCATCAGTTCCTGCCCTGAGTTCCTGCCCT
GTCTTCAGTGATGGGCTGTGATGTGGAAGTGGCAGGGAAATAAACCCTCACTCCCCGGGTTATTTTTCCTCACTGTGTTCTGTCACA
GCAATAGTAAACTAACCGTGACAGAACTTTTAAAGTAGCTCCCTTGGAGCAAGGGTAGGGAACATAGAGGCTTATTTTTTTTTCCTT
TCTCTCTCAAACTGAAGACATACTGAAGGTTTCTGTGGGTACTTAAAGTATTTTGTACTTTATCTTATTTTTTTATTATTTTTGTTT
GTTTGTTTGGTTTTTGGTTTTTGTTTTTTCGAGACAGGGTTTCTCTGTACAGCCCTCGCTGTCCTGGAACTCACTATGTAGACCAGG
CTGGCCTCGAATTCAGAAATCTGCCTGCCTCTGCCTCCCAAGTGCTGGGAATAAAGGTGTGCGCCACCATGCCCGGCTTTATTTTAT
TATTATTATTATTATTATTGAGCTCTGGGAATTTTTATTCTTATAAATAGGACCATATTTCTGAGTGACTGATCTTGTACTGTCTCC
ATTTGGATTTCTTGTCCCCTGCCTCTAAAGTGTCAGAACTCCCTCCTATGAATGGCAAGATCTTGCTGAGTGGACAGCATCCTCAGA
CTCCCCTGAAGCTGGGGAGTAACCACCCCTCACTGCTGTGCTCTTCAGACACCCTCTCAGCAGAGAGGTCTTCTGACATTTAACTTA
ATCAAGATGTTAGTAAAATAAACAGTGTTCTTACACCGTCGGACGGAAACCGCTTGTCACCCCACCCCCACCCACCACTTCAGTGTT
TGGAAAGGAAACTCGGAGTCGCTCTGAGTCCAGTCACGCTCAGTCCTGGCCCTGGTTTCTTCCTGGGTGGGCAGCACTGTCTGCTTG
CCACCCAATCTTCAGTGGCCCAGTTAGGAGTTTTCAGTACTGTCGGGGGGGCGGGGGGGATGATACCATGCCTTTTAAACCACATTT
CAGTTTCGGGGATGTTAAAATGTAAAGGGGGAAAAGTGTGTCTCAGAGCAGACAGGGTCTAGTGAGAACCAATTAAAGTTGGAAGCC
CATCCAGTCCCGTTGCTGTTTGCATCATGACCGCCCCTCCCCCCATCCTCAGTGCGGCTGACAGCGCCTCAAGGAAGTCTGCCTTCA
TTCACTGTGCATGTCATGTGTGTGCCAAGTTTTACATGGAGCTGTGGGACATGCTGTAGACTAGGAAGAGTAATGGGGCCCTCGGAA
CCACAGTTTTAGTGTCTTTAAAAAAAAAAACAGTCTGAGGATTATTTGGGTCACGTGGATCACAGGGAGCATAGGATTCTCGCTGCC
TTTGAAAGAGTGACGAAGGGTTGGTCAGAGGACATGGGGCTCCTGTGTTCTCTCTCTTACCTGGTCCACTCGAAATGGTTCAGTGGG
ACAGAGATCGCTGCCTCACTAATGTGTGCCCTAAATGTTCACATCTAATGAACAGTGTCGTAGGTACTTGGACTCAGCCACTCCACT
CTGAATATTAGTTCATCTCATTCAAGAGTCTACCTAAAGATTATAAGTCACCATTTGAATGACTTTCAGGGTTCCTACCCCATCTCA
ATGTTACAATATTTTAAGCATGTTGGCAGTGGTAGTTCTTTTCCTCGTTAACGCAGATGTTAAAGAAAGCTTTTTTGGTTTGTTTGG
TTTGTTTTTTTTTTTTTTTTTGTCTTGTTTTTCTGATCAAGTAATGTAATTAGACAACCAATTGAAACCATATTTTCGAGAGTAAAC
ACATGGTGCTTTCTCCCTAACGCTGACCCCGTCTCCTCTTCTCTTTCAGTTTTTGGGAATAACGTTTCTTGGAATCGGACTGTGGGC
GTGGAATGAAAAAGTAAGTGGAACTCCTCGGAGAGGAACAGGTTTGGTTCAAGGCCGTGTCTGGTCTCGAGTGTTTCCCCTTGCTCT
TTGATCAGCACAGGGGACAAACAAACCGTTGTCTGATGAGAGGAAGCATTCTTCACGACTGGGTGTCTCAAGTGCTCACGACATTAA
AATAATCAGCAGAGCCACAGTGTAGTGAGTGTGGACCGCGGAGGATGTGACTGGGGTTTGAGTCCAAGGCACAGCTCCACCCAGCCA
CCATGACCTCAGTCGGCTTATAAATGTCACCACGTGACCTGTAACCTTTCATCTCCCTCTCTCGTCTTTTGAATCCTTTTGGAAGAT
GACTTAAATGATAAAAAATGGCTTTTCAGTAGGCCGTGTGGTGGGATGGAAGCCAAATATAAACTTATGTGTAGATATCAGATAAAT
TCATCACCAATGCTGAGGGACATGAACAGGCAGCCACTGGTCATAAATATCAAGAAATGCTCTGAGTCCGTGGTCAGGGATTGCATT
GAAAAAGGAAAATCTAGGCATATCACGTTACATATTCCTAGTCTCAGTGATGACTGGGTTCTGGGCAGTTCCTGTCCTAAGCGGTGC
TCCCAGCTGCATGTTTTGATTTTTCTCTGAATTTCCCAGTTTACCACCGATATCCAAAAGTGGGAATCACCCCCTCTCTCCTCCCTG
TTAGGGGGTGAAATCACTCAAAGCATGTTTTGGCTACACAGTGCTCTTACCTTGTGAGAGTGAGCCCTGTTGGAATTTGGTCAGTGG
TCTGGCAAACCCAGGAAGGAGTTAGCTTATTCTTCAGGAAGGACTTTCTCTTAATCTCCTGAGTATTTGTCCTTCTGAGCCTGGCTT
TATTTCAAGGCAAAGGCTCTTTTGAAGTGTTAAATGTATTAAGAGCTGTTACCAGTAAATAGAAAGGAGACCTAACCAGGGCTTCAG
CCAAATTACAGACTTTGAGGTGGCAATGAGAGTTTGACAGTAGCGGCTCTTCCAGACAGACCCAGGAAACACACTCGGAGTGAAACC
TCAGAGCATGCGTGTGTTATGCAGTGGAAGAGCATTGTCTGGAGTCAGCCCCCAACACTGCATAAAAGGAGGACATGGGTTCCGTTG
GTAAAATGCTTGCCACATGAACATGTGGACCTCAGTCCATCCCCACACTGATGCAAAAAGCCAAGCGTGGCGGCACATATTTTTAAT
TCTATGCTGGAGAGATGGTGACAGGATCCCGGAGCCTGGCTAGTGAGCTTCAGGTACCCATGAGAGACCCTGTCTGAAAAACATGAA
CTGGATGGCAACTAAGAAATGACACCTGAGTAATCTCTGGGCGCCACATGCATGCACATGCATGTGAATGCAGACTTGCACAGCACA
CTGAATATGTGAATGCAAATGCACACCCCTCCGAACACACACACACTGCATAAATGTAATGATATATTAAAGAATAAGGAGAAAAAG
GCACCACTCCTTATATTTATGGAATGCTTCTATAATATATTGACTAGATTATGGAAAATGTACCATTGCCCAGCAAACAGTAACCTT
TTGAGACAAAGCCTTCCTTATACAAAGCAAGACAGCTTAAGTTCCCTGAACAGATAGGTTCATCCCGCGAAACGTCTCTGTTGAATA
CCAATCTTCTCATGGATTGTGGTTAGCTGCTGCTTCATCCCAGTTAAGCCTTGGGATTTGTCCTGGTGGAATCTCAGAAATTCCCCT
TTTAGAGCTCTCTGTCTGGTTAAATGCCAGATAACACGGCTTCTTGCAACTCCTTTATTAAGAGTCCATTTCTGGCAGCTTTGACAG
AGATACCAGAGGGCATTAAATGAACTAGTTGTCTATTCTTGTAATAAACAGGGAGCTGGTCATTTTGAAGATTTTTTTTCCTCTTTA
TGAAATAGAATTACGTTTCTATTTCGTTGGTCTCCTCACATGTCTTTTCTGATGAAATTTAAAAAAAAAAAATTCCAACATTGGCAG
CAGCTCCCAAATACCCGGGCTGCCACCATGTCAGAAAAGGATGGCATCTCAGTCCTTTATCAGGGCCCCTGCCAGTAAGTGGGACGT
TTTTCTTGAGAACTGAAGTTACATGGCCCAAGTCATTCAGCAGAACTTGATGAGCAAGAGGAGGAGGGGCTAAAAGTATCCAGCCAT
ACAGTAGCCCAGCCCGTGGCCCACAGCTCATGGGTCTGGACCTCTGACACAGGGCTATAGCAAGGAGTGCTGCAGAGTCCCCGCCTC
AGCCGGCTTACATTCTAGGAGAGACACTTGCTGGAAGGAGGTCACTGGCCAGGAACTGCATCCAAACACCAGCAAGAAGCATTGAGT
ACTGGCTATTTAGAGAGTGGCAGGAGACCAGTGTCTAGCATTCTACAGTTTGATTGTTGTAGTGAGGACTGAACCCAGGGCTTTGTG
GCTGCTAGGCAAATGCTCTGTTACTGAGCCCTAACCCTGAGATGTCCATTTTGGAGCTGAGAACCTTCCGGAGGAAGTGTCATTAGA
CTCTATGGAGTAAGAGAGATCTGCACAGGAACAGGCTGAGGACATTCCAGGAGGAGGATGGCGAGGACCTTGCTGACTCCGCCTGCC
TAACCGCAGACGCCCCTTGTCTGAGACCTTGGAGTCTTCAGCAGGCCAGATCTCTGGTTCAGAGTGAACCCTAGTCAGGTGGAGGGT
GGAAAGTGGCTGAATCTGCTGGGTTAACATCAGTGTTAGTCAGTAAACCCAGACAAGGCTGTGGCGTTTAGTTTGAAGGAGGAGGCA
AGGACTTTGAACTGCGTCAAAGCTGGAGTGATGTTAAATCCACGGTGGGGATCAGATTTGCTTAATGTGAAAAACTTGAAGCACACT
TAGGTGGAGCACAGGAGCAGTGGAGGCTAAAGTAACTACCCTTCAAAGTTAGTATCTCTCAGTCCGGGTTAATAGAGCAGGTGAATG
CTAGAAAGAATAATTTTATGTAAATTCCAGTTGCCCTTGAAGATACAAGGAATGGCTGCCTCTCCAGGGGAAGCTGGTCTGCATTTT
GTTTTCTGAGATCACAAGATCTGTTCCTTAGAAGGAAACTTAGCAGTTACATAGCGTGAATCAGGTCTCCAACACCCTTGGTGACCT
GTAGCCGTAGAGAACTCATTACTTCTGATAACATTATTGAGCACATAGCCTGTGACGGAGCCTGATACTGAATCTAGGAAGATGAAG
CAAAGAGTTAGAGTTCTCAGATGCAGGAAGGGTATGTGTATACTCACAAAAGACATGCATATGAGAATAACTGCATTCAATGGTGGT
CGGAGTGGTTGTGCATTGGAGAGATAAACGACATGCATACGATAATGCTGCAGTCAGCGCTGGTTGGGGTAGTTGTGCATTTCAGAG
TTCCCACAGTGCCCTACCCTCCCCGTTTCTGTATTTGGGATACTTTTACTATTCAAATCACTATGGATTTTCACTCTGTGTTGGATC
TCTCCTCTTAGCTCTTGGGGATTTTTAGGATAGAGCTCTGCTTCCTTTGGGGTATGCAGGTAGAGCATCTGTAAGAAACACATAGCC
TCAGAGGACATTTTTCCCAAAGCTGTGGGAATTGCCAGGCCGGGGGCCACCATGGCTGTGTTCCCGCCTTCATTACTATGGAGGACG
CGATGGATAAAGAATACCTCACGCCCAACACCCTAGTCCCCGGTGGGCAACACCGTGGACCTGCCCATGGCCCTAGATAACTATGAC
AACCAGGCCATAGAGAGCACATATGTGTGTGCAGATGATAATGAAGCCCATGGAATAATGTGTGGAACAACCTAGTTGTAATCGGCT
GAATTTAGCTTAAAAACTCCGGTCTGGCATGGCAAAATCACTCACCTCCTGAGTGCCCAGCGGAGAAACTTCCAGCCGAGAAATAAG
TCTGGTAGACCCCACGCAGACACGTTCATCTGCTGCTTAGAGAGCAAAGCCTACAGGACTCAGCTCAGGTCCAGAGGCTCCCAAGTA
AGGAACATGAGTCTCGCCTTACCTGCGCACTGCCACTGGGAGCTAAGGAAACACTTGCCAAACTGGAAAGGTTAGTGAGAGAGTGTT
TATGTTAACGAACGAATGTGGGCCCTTGTTACCATTTATTTGCTTTTTGACATTTACGAGTAAATAAAAGTACTAAAACCTTTTCCA
CCAGGTTTTGAGGAATTTGTCTCTGCCACGCTTTACAGCCCAAATCGCGATAGCATCCTTTCTCTGGGTCGTCCTCCCATGCTATGG
GCTGGAACCCCACCACCATCCTCTTGGACATGCACCTCTGGCCTCCCTATAGCCTACCCTTTCCACCACTGAGGGGACAATTGAGGA
GCCATAGAAAGTATGCCTATGGCCTGGAGAGGTGGCTTAGCAGTTAACAGCACTTGCTATTCTTGCAGAAGGCCTGGGTTCCTTTTT
CCAGCACTTACAAGGAAGTTCACGTCTTTCTGTAACTACAGTTCCAGGAGATCGTATGCAGGCACTTGATATACATGGTGCAATACA
TACATACATGCACAGGCATAAAATATAAATAAATAAACCTTTAACATTTAAAAATATGTATATATTTAATATTAATTTTATATAAGA
ATATATTTAATATAACTATATTATATATATACATACAATATATTAAAAAGGAAATATGTACTATGTTTTGGGTATCCATGGAGTTTG
AGATCCACTGTCCATCCCTGAGTCCACAGCTTCCTGATAGTTCCAGCTTGTTATCAGCCCCAGCTGCCTTACCACGTAACCCACACA
TCTATCTAGGTTTGGGGCTCAGTGGACAATTCTAGGCATGCCTACCATCACATCCACCTTTTCTACTTCTAGAGCCCAAACCGTTCC
TCTAGAAGGACACCCTTCCGCTCAGTGTGGATTCGGCCACACACCTGGTCAGTGTCCTTCACTTCCTCCTCCTCCTCCTCCTCACTC
AAGCCTTCCTCCTTCAGCCTTCCTTCTCCCTCTGTGAATATGAAGCTGTAACTTGAAAAGGGCTGGAGTGGTCCCGTGAACACTGGC
TGCACAGGCCTCAATCGATCGTTCCAGTGGCTGTCACTAATGAAGGCCACTGGAGGAGTGTTGATTGGGTGAAGGACGCAGCTCAAG
TCACACCGTTAGAATTCTCCTTTGAGATCGACAGGCACTCGTTCAGGTGATGTGTACCCTGGGGTCGGGCATCCTCTCCGTCATCGG
CAGCATCCTTTCCTTTGTCATCCCTCCATGTCTTTGCGAGCCCGGGAGCTTAGTTGGACCTTCTGTTTCCTTACTGAGCCCTTGCTA
GGTGTCCCCACATAGTTCAATCTCATGCACAAACCCAGTGTGGTTTCTCATCCATGGGGCTCTCAGAAACCCATCCCAGCTCCGGGT
TGCAGTGCCTCACTCTCTCTTCTGCTCACTTGATCCTAGGAACTCATACTCTGCTGCTTTTCTTCCCTCATAGCCTTGCCAATTCCA
CCTCTGCCCTGAGACCACCACTGCAGGTATCTGCAGAGGCCAGAAGAGGCCCTTGGATGCCCTAGAAATGGAGTATGGATTAGCTGC
TTGATGTGGGTGCCGAGAACTGTACCCAGGTCCTCTGCAGAACAGTCTGTGCTCTTTACTGCCAAGGCCTCTCTCCTGTCCCTGAAT
GGCACCTGTTAGTGTGATGATCGAGAAATGCTCTCCAGGGGCTGGAGATGTAGCTCGGGTGGCATAGTGGTTCCCTGGGGTTCTCAA
GTCCCTGGGGGCCATCTCTGGTACATAATCCTGATATGGTAATCTATACCTTTATTCCTATCACTCTGGAGGTGGATTTGGGAGGGC
CATGAGTTAAAGGCAGTTCTTGGCTTTGTAGTTGAGGCTACATGAAATCCCATCTCAAAGAAAGAAAAAGTGGGAAATGGGGAAACA
AGAAAGAAAACATGTTGTTTACACAGGGCATCCACAGGGTGGTGGATGCCTGTAATTCCAGCACCTGGCAGGGTAGTGTAAGAAAGA
TGGTTATAAATTTGAGGTCATCTGAGGCCACATAACAAGACCCAGTATTAAATGCAGTGATAAGAAATATGATCTCTGAACCATTCA
TCAGTGAGGAGATGAACATTATTTTAAGCCCTGGCTATTCCATGGCCTCTCTCATTGAGCAACAGAAGTCAGTAGTTCCCTAAGATT
TCTCGTGAATGGACTACTTTCACATTTCTTACTACATAAAAGGAGGTAAATTACACCTGGTTTGTTTCTGATATCTGGTATGAGTGA
CACAGCCTTTGAAATAGCAGGCTTGGAAGTTCTGGGAAATGGCCTCTCGTTTCTCTTGTTAAGAAGGGGACTCCGTATGATACCTTG
TTTTGTTAGAGAATTAGGTGTTTTCCCTCGCCTGCTTTTGTCTGCTTTTCTGCTCATTATTACTTAGTTCCCGGCAGGCAGGGCAAG
GGGAGGAGTGGCCTCCCTAACTGTTGAAAGGGAGAGTTTTATGGCTGTTAACAGGGGATGAGAGGACTCCACTTCCCAGAATAGGAA
AGAGCCCTTCCTTGATCCTGCCTGTGCCTTTTGGATTTCCAGTCTCCCCTGCCTTCTCTAACACAGCCTCTGCTACTAGGCAGACAA
CTTCTGGGGCCGTGCTTGTTATTCAGTAGGGAATACCAAGGGGACAGGACTTACAGCTCAGTGGCCATGTGACCTAGATAGACATCC
CTACAGGGGAAGGGAGGTTTCTCCCTTCAGTGATTTGGCAGGTTGACCTCTCTCACTTTCTGTCTGTTTCCCCACCTGGAGTACCCA
GGTTATAGGTTTGTCATGAGAGTAAAGTAATGCAGGTCTCAGCAGGGTGGCTCTCAGGACTGGGCCTTCTCCACAACTGCCCCACCA
GCTGCGTCCGTGGTGCCTAGCCACTGCCTACATGGAGCTTCCTGGTACAGAAGTGCCTGGGCTCTCCTCTCCTGAGCATCCGCAGGG
CAGGTCCAGTCCCATCCAGACACCCTATTCTGTCTTCGTCATTTGTCACATCCAGAGACCCTGTTGACCAAGTTGTTTCTGCGTCTC
AAGTCTGCTGACATGGCCTTCCATCTCCGAGCACATGGCAGCACTCTTGGATAGCTGCCCATAGCCTCTCCCTGTGGACAGGCTGTA
GGCTGTCACTGGATCTTACTCTAAATGGCCAAGACTGATGGAGTCCTTGGCATCTCAACTTCAGACTGCAGACTAAGACAAGATACT
ACACTGTGGACTAAATGACGAATGATAGCCCTTGGTCCCTCACAGCCCTGAGAAGGATGGCCGTCTACATCAAGGCGCTGGCTGGTT
CCAFTCCAAGAGAAGACTGTCTGTCGTCCTAGTCTGCAGATGGGTGAATCCGCACTGCATCTGCACAGGGCAGGATGAGAGAGGCCA
GGCTCTGCTCTCTTCCTCTTCTTACAAGGACCCCTGTCAGGGCATTGGGGTTCTAGTCTCATGGCCTTCTCCTAGCCTAATATCCTT
CAAGATATCATCTTCAACAATTGGTGGTGATTATGTTTCAACATACTAATGGGCTGGAGAGGCGTTCAGTCCTAAACAGAGCTTCTA
TTGCTTTTTCTGTTTAAATATTCTGTTTAAGACTAGAATGTTCCTAAGTATATTTTCTGCCTTCTAAATAGATACATGAAAGAATCC
AGCAAGCGGCAAGGAACACAGGGACCTTAGCATGTGGGGACACTTCTGTGTGGAGCTGTGTCTGCGGGACAGTCACTCACTCGTGGG
GGCGGGGACTGTGTCTTCCCTACCTGTGAGCAATGCCAGCCTGTGGAACTTGGCCCAGCTGTCCTCCCAACAGCTGCTTGACTGCTA
GATTCTGTATAGGCTTAAAAGATGATCGTAACCCTCTTCATCTCTTCAAAGATACCTCATGACGATTTTTTTTTTTTAATTTTGCAA
GAAAGGATTACTTGAGTCACTCTGATGAACTTGCTTGACAAGGTAAAATGGAAGGGATCTTGTGCCTTTGAAGCAGTTGGCTTTCCT
GCTTGTGAGCTTTTCTACTAGGTGTGAGCTAGGCTGCTCTCGCCCCATCACATCTCCTGACACGCTCTCCTGGCACTCTTCATCCCC
TACCCCCTCTGGCCTCTGCGCAGTTCATCCATCCCGTAGATGGAGACTTGGGTTTTTACAAAATGACTGCCATGGCACATTCTTGGA
GGGCCTTTGATGTGTTTCCATAGTCCCCTGCCTGGTCCTTCTGCGGACATCCTTCAGAAACACATTTCCAATTCTCCAGGACAACCC
CCTCTATGGGGAGCTGCATGTTCTTTGAAATGTCGCACCCGCCATTGTCAGCAATGGAAATGGCTCCTTCCCTCGTATCCTGGGGAG
GTTTTCAGGCCCACCATTACGGTAAGGCCTGGTGTGTGGGGCATAGGTGAATCAGGACCCAGTGTCAGAAATATCTCAGCGGAATTC
AACTGGTCTGGGTTTGTACACATTTCATATGCCTGCCTCTCTGTTTCTCGTCCAGCCGTTGGAAGGAATCTGCTTGTTTATGAAAAG
ACAGGAGAGCCCCAGACCCCGGGGTGCGTTCCTGCTATTTTTTTGCAGTTCTTCTCTGTGTGGGTTGACAGAAGAAACTGCAGCCTG
CCAGGTGGCACACAGAGGCTGCTCTGTGCTGGCGCTGAGGAAAACCACAGACACTCCTGAAGTGTATTGGCTTGGAACACACCGCAG
CTTTGTTTGCCTTTTTTTGTTTCTGGCGCTTGCAAAAAGCAACTTCTTTGAAGAAGAAATTGTTACCCAGAACAGACTCCTCTCTTG
CGGGAACCGTGAAGAAGATGCTGCTTCAGCAGCAGAGATGGCTGGGCGGATGAGGTGCTGGTGACGCGGTGGGGACGAAGGTTGGCG
GATGTTTCCACCCTCTTACCGGAGCTTATGCGGACAGACTGGAGTTGAGCAACCATAAGAAGGTCACTGCAGAGTTCTGAGTCCAAC
CGCCACCATGTGCTGTCAGCTCCCTGGCTGCAGAAGGATCCAGCTGCTGCTTGGGCAGGGTCCCCTTGAGGCTGTGACTTCTCCGTC
TTCTTCCTCCCGCTGTGCCGCTTCTGGGTACCCATAGAAGCTTTTACCTAGTCCTGCTTTTGCTCCCTCCTGGTTCTCCCCTCTGCC
CTCCTCTGTACAAGCTTCTATTATTTTTACCCCCTACCCTGTCCCGCAAAGATCCAATTAGCGAGCTCCAGATACTTTTGCCTCACT
TTGTGAAATCAACAGAGTGGCCTCAGATTTCCTTAGGCACCAGGCAGGGGAAGTACTTGGAGCAAGAGATTCCTCTCTGGTAGCGCG
TGCGTGCGTGCGTATGTGTATACGTGCGAGTGAGAGCGAGTGTCTTAGGGTTTCTTTTGCTGAGACAAAATAGCATGACCAAAAAGC
AAGTTGGGCAGGAAAGGGTTTATTCAGCTTCCACTTCCACATTGCTACTTATCACCAGAGGAAGTCAGGGCAGGAACTGAAGCAGGA
TCTTGGAAACAGGAGCTGATACAGAGGCCATGGAGGTGCACCGCTTACTGGCTTCCTGCTTCCTTGCTTCCTTGCTGTGGCTTGATC
ACCCTGCTTTCTTACAGAACCCAGGACCGCTAGCCCAGGGGATGCCAACTCCCACGTTGATCACTAAAGGAGGAAATGCCTTACAGC
TGGATCTCCTGGAGTCATTTCCTCAGCTGGCTAACTGGGCTCCTTCCTCTCTGCTCACTCCAGCTTGTGTCGAGTTGACACACAAAC
CCAGCCAGAGCATCCTTCCTCTCAGATGTACCCGTGGATGTGGTGGCATCCCCTTGTCCTAACGTAATAGAGATGTGGATGATAGAG
CTAAGCTGTCACTGGATTCTTTTCTGTGCCAGGTATTTCGCGCAGAGTGTTTGCATTTACTTGATCTATCTCGTTCTCATAATCACC
TTATGGCATATGCTATAATTTCACGGATGTGTAATTGAGGCTTAGGCAGGTGAGGCGGCTTGCCTACAATCACATAGGCAGTAAAGC
AGAGAAACTGAGATGCAAAACAATTTTGCCAGATGAAAGATAATAACAAGTGCTAATGAAGGTGTGGAAAGTGAACCCTTGCACAGT
GTTGGTAAAAATATAAATTAGAAGAGCCGTTATGAAGAAAGGAATAGAGGTCCCTCAAAAAATGAATTAATCTATAAAGCTAGGAGC
CATGTGAAATAGTATATAGAGATAATCCAAACATCTAAGAGGCTACAACAGGAGGATCACACTGAGTTCAAGCCCTGCCTGGATTGC
ACGTTGACTTTGAGGCCAACCTGAACTACATACTGACATTGCTTCAAATGAAGAATTAAAAACAGAATTGCCGTGTCTGGGGAGGGA
GGTGGGTCAGAGGGCAAAGTGGTTACTGTGCACCGGCAAGGACCTAAATCCAGATCCCCAGCGTCCGTTAAGATCAGGACAAGATGG
CACACACTTTTAAGTCTGGCACTGGGAGGTCACAAGATGCCAGGATCTCACTAGACAGTATAGCCAAAATGGTAAACTCCAGGTTCA
GGAAAGGACTCTGTCTCTAAAAACAAACAAACAAAAATGATGGAGAAGCAGTTGAAAAAAAAAAAAAAAAAAGTCTAGTGTCAACGC
CTGTCTTCTACATACTTCCCACACATCAGGTTGGCACACATACATGCATACGTATACATAGCTCTGAAGAAAAAAAAAAAGACAAAA
TAACAAAAGCTGGTTGTCCAGTTTCTGGAGTGGCGATTTGTTGAAAACACTTGTTGCTTTCACGTTTGTAATGGACTGAGCTGTATC
TCGTGTTTAGAGCAGCGCTTTATGTTATGAGTGATTCGGCTTTCCTCTCTTGCTTGCTGGGACAGCCGTAATCTGAGCTGGATTGCA
GCATGAATATGAGGCAAGTTTAGGAATGTGTGCTAATTTACATCAGCTCCTATTATTATAATCTTAATATTCTTAATCGCACACACA
CACACACACACACACACACACACACCACCATTATTTTCCCTTATTCTCATCCCTTCTCTTTCTGCATCTGCTTCCCCCCGACCCCCC
AAGGGGGTCAGAATCTCCCTTCCTTGTCACCCTAAGCACAGGTGACCAGTCACTCCACTCTCCCTCCACCTAGACTCAGCATCTCAG
AGTGGCATTTTCTGACTTCTGCCAAGGTCAACATCATTGGCCTCACTACCCCCTCCATGCCTGGAGCATACAGGTAGGAGAAGGATA
AAAACTTGTCACATTCCAGTGCTAGGCCATGTGTGTGTGCTTGTCATGTCAGCTCTGGCTATTGGAGGGACAGTCGCCAGAGGACGG
TTTGTGCTCGTTTTTAAAAAATAAGTTTCAAGATTTAAAAGTTAGTGTTTAAACTCCAAAGCTAAGATAGTCAACTTTACGTGCAGA
ATTAGGACGTAAGCCGTGGTAGTATTGCTGGCCAGTGATGTCGCTACCAGAGTCCCCAGGTTCCATCCAGACTGCTCATCTCTTTGC
TAATGCTGAGTTGAAAGCCAGTTCTCATGAATGAGCCAGGACCAGCCTTAATTGCTGCTGACATGGGTCACCAGCTAGCCTGGAGAA
GGCATCAGTTGAGACGTAGTACTTCTTTCACTCTTCTTCTCTGATGTCCCTAAACAGCCCTTCCCTCTTTCCAGCATGAAGTGTGAG
CTTTAGTTTTAGACAGGCTCTGTTAAAATCTTCAGGAGCATCAAAGCAAGACTCTCTGGGTCCAGATTAAAAACTCAAGTAGAACTT
TCTCTTTTAGTCCTTGTTAGTTTTCTTTCCACCTTGATATTTTGTTTCGAGTTTCCTGTATGGCCTGGCTTTGAATTCACTGTGTAG
CTAAGGATGGCCGTGAACTTCCACTCCTTGCACCTCCATCCCAAAGGGCGGGGGCCACCAGCTTGTATGCCCAGGAAATAGCTGGAA
TCCCCCCTGCTCTTGTGCTGCTTTTCTTTGTCTTTTTTAAGATTTATTTATTTTATGTACATGAGTACACTGTAACTGTCTACAGAC
ACACCAGAAGAGAACATCGGATCCCATTACAGACGTTTGTGAGCCACCATGTGGTTGCTGGGATTTGAACTCAGGACCTCTGGAAGA
GCAGTCAGTGCTCTTGACCTCTGAGCCATCTCTCTAGCCCCCATGTGGTGTTTTTCTTAAGCACCACCTTTGTATTTCTCCATGTGG
TAAAAATTAGCCCGACACATGGTTGTACGTCGCTCCGTGGCTCTCGGTGTGCACTTTCCTAGCTCTGGTCCCCAGAGAGAAGAGCCG
GTCATAAATGTCTGCATCCCAGCGGGAGGACTTGAGACTTCCCTGCTCTCTGGGGTGGGGAGGGGCTCTGGAGAAGCTGGGGAGCAC
TGACAGGGATGAAGAGGATGAAGAGGAGGAGTCCCCACAGAAGAGGGTGGGCGCCACAGGTTTCTGGTAACCAGGTAACTGCTGTTT
TCTCATCACAGGCTCCTCAGGATTCACTGCAGATCTAAGCACCTACTCTACACTTGGCTGACTTACCTGTGACCTTAGAGTTGTAGG
TTTTTTTTTTTTTAAGCTTGCATTATCATTATTATTCAGCAGGCATGGACTAGCACTTCTTCCTAATTTGTCATATTCGGCTCCCCA
GAATTAAAAGTCCAGGGAGAGCGCCTCACGCTTCTGACACCAGAAAAATGACGCCTTGGCTCCCATCACTTTTGTAATCTAAAAAAT
GAGGAATGTATGCGGTTTGTTTTGAAGTCCTGTGTAATCAGAAATAGACCAGCTCTCTGAAGCAGTACTGACAGATGGGAGCATCAG
CCTGCTTTCCCTCCGCATCGAGATTCTAACCTCATTCCAGGTGACAGAGAGCATACTGAATATACTGTATGCTTGAAGAGTACCGAG
CGTGGGTGTGCTATATTCTCCCCAGCAGAAAGTATAACTGTGGGGTAACGAGTGTTATATGGATTTATTCATTCCAACACACACACA
TATACACACACACACACTAAGAACATCCAACTTATTAGGTAATGTAATATTTATTTGCTGTTTATATTTTGGGAACGCTCCTCATTG
TCTCTCTCTCTGTCTCTGTCTCTGTCTGTCTGTCTCTTGCTCACTCGCTCTCTCTTGCTCTGTGTGTGTGTGTGTGTGTGTGTGTGT
GTGTGTGTGTGTGTAGCCCCAGGCTAGTTTCAATGTCTCTAGCCTAGGCTGGTCTCCAACTTGAGATGTCTTGCTTTCACCCTGAAT
GCTGGGACTGTAGACATGTACCACCACAATGGAAGTGTATGTAAGCTCATCTGTTCCTATGGTATAAGGTTTTCTTTTACCCCACTC
TGGGGAAAAAAGAAGTCTGAATTAATTTACAACTGATAACAACTTGGGTGTGCTCTGAACCCATCAAAAGTAAAGTCCTCAGGGGTC
CTCCCTGTGTTGCATGCACAGAGCCCCTGGCACTTGGTGGTACCTGTCTCCAGCACTGTCTTTTCCTGGAATTTAGAATTAGATAAG
GAAACTAGGCTCTCAAATTATCATCGGTTTCCATGGAAACAGTTGAAAGCCCCTCAATTAAAAGTGTCCCTGCAGGCTAGGGAGAGT
ATTCAAATGAGCTGCTCTGAGAAGCCAAGTTATCCTCAAAGTGAAGCCAGAGAGGACGGAGGAGCAAGGAGTCTGCTAGAGCACCAG
TTGCGATCTCTATTATTTTTTTTCCATTCAGGGAAGAATTTTATCTTGGTATTTCATTTGTAGCATGTTTGTTGGCTTTGGCAGAGC
CACCTAGATAATTGTCCTGTTATCTACTAAGTTTTTTATGCTCGCCCATGGATCACGGAAGTGTTTGAGAACGCTCTGAGCTTTGCT
CACACATGAATATGAGTCACAGATAAAAAGCCCATTTAAAAAAATTGATGATATCCTAGAAGAATACTAAGTTGGAAAGAGTCTGTG
GGTCATTAAGAACTAACTTCCCACCCATTTGGGATTCCTTGCTAGAGTGCAGTAACAGAGGTTGTTCAAGGTATACTTCAAACAAAA
AAAAAAGAATTTATTTATTTCAAACGTATGAGCGAGTTACATGCATGTATGTCTGTTTGCCGCATGCATGATGCATGGTGTCCTCAG
AAGTCAAAAGTGGACATTGGATTCCCTGGAAGTAGAATTATGGGTGACTGCACTATGTAGGTGCTGGGTTCTCTACAAGAACAGCAA
GTGCTAACCACGGAGCCATCTCTTCAGTCCCCTCACATTATACTACTAAGGAAAGCAGTGTGTGTGTGTGTGTGTGTGTGTGTGTGT
GTGTGTGTGTTTCATGGTAAGTGCTGGAGAGATGGCCCAGTGGTTGACAGCACTTGTTGCTCTTCCAGAGGTCCTGTGTTTAGTTCC
TAGCACCAACACGGCAGTTCACTACCACTTGTGACTCCAGTTCCAGGGCTCTGACATTGTCTTCTGACCTCCATGGGCACTAGGCAT
GCATGTGCTACCCATAAATGCAGCCCAAACACTCGTGTACATTATAAAAGAAGATTTAAAAAGACAGTAATTGTATACCTGTCAAGC
CAGGTGTGAGATCTCTGCTTGGAAGTTGGCAGTAGAGACTGCAAACTCAGGGCCTTGCTAAACCAGCACCTCATGCTTCCAGAAGTC
ACATAGAAAAACTGGATGTAGCAGCACACGCCTGTACTTAATACTAGGGAGGCAGAGATGACATAACCTTAGGGCTTGTTGGCCAGC
CAGCCTAGCGGAATGTATGAGCCAGTTCACTGAGAGGCAGTTCGCAAAAGCTAAGATGGATCATGATTGAAGTTCAACACTCTAGTC
CACAAACACAGTTGCAGACTCATACACACGTTTGACACACACACACAAACACATAAACTCACATGCGCACGCATCTGTTTGTTGTCC
CAAGAAACACACTTTATCATCAGAGACAAAAACCTGAAGATGAAAGGTTTTTCAGCCCTGTGGAACTGGAAAGAAATCAGGGTATAT
CAGAAATATCTGACAAGATAGACTTTAAACCACTTAGATGGAAGACATAAAAAAGTTACTTAGTACTAATTAAAAGAAAAAAATCAC
AATTTGAAACATGTGTATACCAGACATGTGTGTATCTAATTTTATAAAACAAACACTGTTGGATTCAAAGAGGCAGATTAACCCCCA
ACCCAGTAATAGTTAGTAATGCCAGCACTCTACTCTTACCAATAGGCAGTCCAACAAAACCTAGAAACATCTAAGTTAAATGACACA
GGTCAGATGGACCTAAGAGACATCTGTGGAATGGTTCACTTAAACCCTGTAGAGTCCACATTCTTTTCGAAGCTCAAGGAACCATTG
TATACTGTCTGATGACAGTGGAGTGAACCTTGAGATGAACAGCAAGAGGAACTGCAGAGTGTAGAGCTGAGGACCCAGGACACATTC
CTGGATGAAAGCAGTCCTAAGCAGTTAATTCATAGCTACAAGTATGTACACTTAGAAATTAGAGCTCAGGGTTGAAGAGATCGCTCA
GCAGCTAAGAGAAGAGCCCTGGCTGCTCTTCCAGGGACCTGGGTTTTAATTTCCAGCACGCTCATGGCGGACTGCAACTGTAACTCC
ATGTCTGGAGAGATCTGTCTGACACGTCTTCTGGCCTCTGTGGCACAAATGTAGGGCACAGACAAGCATTCAAACTGAACACTTGTA
CACATCAGAAACATTTAAAAGTTATTTTTAAAGAAGAAACCAGCTAGTGATACATGTCAGGGCCTTGGTAAACAAGAACAAGCCAAA
CATAAAACCAACAGATGGGAACAAAGACTCAGGATCAGGGCAGATGAGCGTCATGAAACACTCAAATGACAAAGAATCAGTGAAAAA
CAGGGCTTTGGAAAAATAAACAAGATTCACATGCTCTTAGCCAAACCAAAAGGAAATCCATGCAAATTAATAAAATTAGAGATAAAA
AGGGAGGCATTGTAAAAGACACCAAATGAAAGTCGGAAAATCAATAGGACCCAGCCCTAAAACTTTTGGAAATGAATTTCTAGACTC
ATATGGCCTTCCAAAACTAAACTAAGTTTAGATAAATAACTTAGATCTATTTTTAAATATTTAAACAACAACAATGACAATAATAAT
AATAAACAACTGAATTATAATTAATAATAATAATAATAATAATAATAATAGAAAACAACTCTGAATAAAGTAAAATAAAATAAAGGC
CAGGCCCAGATGAGTTCGTTCCGTTCTACCAAGCCTTTAAATAAGCATACTATTCCAAATTATTCCATAACATAGAAGAGGAGGGAA
TACTGTCATTTCACGAAGTCCCATTACTCAGGTACCAAATCCTCATAAAGGTACAGAAACAAGAAGACAAGAGAGAAAGAAAGGGAG
AGAGAAGGAAAGGAAGAAAGACCGTAGACTAATCTCCCTAACGAACGCATTCTCAGTGAAGTCCGTGCAAAGCAAGCGCAAGAACAC
GTCCAAAAGGCCGTCGTCCACATAATCAAGTTGGCTCCGTTTGAGAGATGCAACGGTGGTTAACCATACATACGTCAGTAAATGTAG
TCATCACAGGCATGGCCTCGAGGGTGGAAATCACGTGATCATCACAGTACATACGGATTGGCAAAGCCCCCATCCCTTCATGATAAA
AGCTCTCGAGGAGTCCAGAAAGATGGCTCAACCATTTAGAGCATGTACAGCCTTGCAGAAGACAGGTCAGTTCCTAATACCCATATC
AGGCTTGCTGCCTCTAACTCCAGTTCTGGAGCAGTCTGACATGAGTGGCCTCTGTGGGCACCTGCAAGCACTTGTGTACACACACAC
ACACACACACACACACACACACACACAAACGATTTAAGGAACTCAGTAGAAGGACCATAGCTCAACAATGAAGGCTGTATGTGAGAA
ACCTATAGCTAGCATTATCCTAAATGGACAAAAGCTGAATCGTTTCCACTGAAATCAGGAACAAACCAGGAGCGCCTGCCTTCTCCA
CCCTGCGGTTTGGGAGTGCGCACACACGTGTGCGGTATACATATGCAGAGAACAGAGTTGTACACACGTGTGCAGTATGTGTATGCA
GAGAAGAGAGCTTCATTTTCCCTTAGATTTCGAGAGAGGGTACCCAATGAAATGGGCAGCTTATCTTTGTAGTCAATCTGGCCACCA
AGCCAAATCTACTTGTTCCCACCACCCCACTCCCATTTTAGAGTTAGAGTCACACAACTCGCTTTTATGTGCATGCCAGAGAGCTCC
AGACCCTTCCACTTGCAGAGTGAGCACTTGTTCACTCAGCTATCTCCTCAGACTCCATCCCCGCCCCGACCCCCAACACACACATTT
CTCACTCAGTTAAATATAGTACTTAAAAGTCTTAGCTAGAGCAATAAGTTAAGATAAGGAAATACAAACAAGAAATCAGTTTCCTTA
TTTACAGCTGATCCGATCTATACACGAGAGGCTCTGAAAACTCCAACAGGAAACTCAGAGCCGATGAACACTTCCATCAAAGTAAAA
GGATACAAAAAATTCACATACAACATCAACTGCCTTCCTCTATACCATGGCAAACGTACTGAGAGGGTAGACAGAGAAACAGCCTCA
TTCCCAGCCATCCAAACAAGTACCAAGAATAAATGTAACCACAGCGGTGAAAGACCTCTACCATGAGAACTTTAACACACAAAGAAA
GATGGAGAGACCTCCCATGCTCATGGATTGGAAACGTTAATATTGCGAATACGCTACCAGAGGCAATCACCAGAATTCAGTACCTGT
CAAAATTCCAACACTATTTTTTTCTCACAGAAATAGAAGAAAAAAAATTAAAGTCATCTGGAAGCACAAAAGATCTCAGATAGACAA
TAGAATTTTCAGGAAAAAAGAATTTCAGGTTGATTCATAGCCTATTTTAGGGACGACAGGGCGGTAGTAACAAAACAGCATGGTACT
GCACAGAAACAGACGTCTATATCAGTCGAACAGAATAGAGGAGCTGGGAGCAGAGTCAAGCAGTTACAGCTGCCAACATTTTTGACA
AGATTTTAGTACAATATAGTGGAGAAAAGTCTCTTTTCAACAAATGGTGGAAGATCGAAGCTAGATCCTTATCTTTTGCTGTGCACA
AAAAAATCCATTCCAAATGGATCCAAGACCTTAGCATAAGCCCTGAAAACTCGGAAACCATTGGGGAAGAAAACATTTCCAGACATT
GGCATGGGGTGGGAGAGGCGGCTTAGTGGGTGAGAGTGCTTGCTGTTCTTCCAAAGGACTGGAGTTTGGTTCCCAGCACACACGTCT
GGCAGCTCACATGGAACTCCAGCTCCAAGGGCTTTGAATCCTCTTCTGGCCTCGTCAGGTGCCTGCGTATGTGCATATACACACATG
TAAAGTTAAAAAAAAATCTTAAAAAAAAGATATTGACATCCGCCAACACTCAGTAAGGACTCAGAAAGCAATTGACAGATGGGATTT
CATGAAATGACCAAACTTCTGCACAGCCAAGGAAACAGCAGAGTGAAGACACGGCAACATGGGAGAGAATCTTTGCCGTCCCATGGA
AGACTAACCTCTAGAATATACAAAGAAATAGAAAATGCTACATGGGGCTGGCACGATGGCTCAGCGGGTAAGAGCATTGACTGCTCT
TCCAAAGGCCCTGAGTTCAAATCCCAGCAACCACATGATGGCTCACAACCATTTGTAATGGGATCTGACACCCTCTTCTGGTGTGTC
TGAAGACAGCAACAGTGTACTTATCTATAATAATAAATAAATCTTTAATGCCAAATGAACCAAACAATAAATGGGCTGGTGGACAGA
CAGTTACCAAAAGAGGAAATACAAATGGCCAATAAACATGAAAAATTGTTCAACTGGCCATCAGAGAACTGTAAATAAAAGCTAAAT
TGAGATCCTATGAGAGACAGAGTAGCAGTCATTGAGACAACAATACCAGACAGTGGTTTGTATGGGAACAAAGGAGAGCTCATATGC
CATGCTAGTGGGATATGAACTAATCAGGCCACTGAGACTCAGTGTGGAGTGTCCTCAAAAGACAGAAAAATGAGACTGCCATATGCC
TTAGCTGGCTACCTGTCCAATCCACACAGGACAAGGACACTTGCATGGCAGTGTTCACTACGGCACTGTTCTCCATAACCAAGGCAT
GGAGCCAAGTTCACCAGTATAGAAATGCATAAAGAGAATGCAGTGCGTACACACAATGGATTGCTTTCGTCCATGAAGAATGATGAA
ATTGTCATTTGCAGGAAATGAGTGATGGCGATCATCCAGTTAGGCAGATGAGTTCAGCCTCAGAATGACGAATTCCAATGTTCTCTC
TCTCTTATGGGTATTAGACATTGCATATACATAAAATCATGTGTGTACAGACGATGTGAAAGTGAAGGTCAAACTGTCTAGGGAAAC
TGAGCGGACTAGAGGGATGGGGAGAGCCAGGAAGAGGACGTTAGCAGGATTGGGAGTCAGAGGTGGAATTCTAGCTCAATGTACATT
ATGTAGTTATACTTGCCTGATGTAACCAAATACAATTTAAGAATCTAATAGGGCTGGAGATAGCTCAGTGAGTAAGATGCTTGGTGG
ACAAGCATGCGCGCCTCAGCATTCCACCCTTGTCATTCATGTAAGAAGACAAACATGTGCCTTCTGTTTGTAATCCAGAGCTGGGGA
GTTGGAGACAAACAGAACCCTGGGGCCTTTGGCCCAGACATGCCAGGCAAATCATCCAGCCCCAAGCTCCAGGGAGGTAACCTTGTC
TCAAAAGATAAATAAAATTAAAAACTAAATGAAGAGGATCGTGCCTGAAGAATAACAAACACCCTCTGGTTTACCTCTGGTGTACAC
ACACACACACACACACACACAGGCACACACACACACACCACTGCTCATATACACATACATAATGCACATATGTACACACAGAGACAC
TGTAAACACTTAATACATTTAAGACAGCTGAAAAGGAATGACAAATTGCCAATGTTGATGTTATGCTAATTACTTCAATCTGAACAC
CACACACTGCATGTAGCCACCGACTGAAATTTGACGTTGTACCTCCACAGTACCATGTGCAATTCCAATACATCAATTTAAAATATT
TAATTAAAAATTGTGAGGAAAGAGCCGGGCGTGGTGGCGTACGCCTTTAATCCCAGCACTCAGAGGCAGGCGCAGGCGGATTTCTGA
GTTCGAGGCCAGCCTGGTCTACAAAGTGAGTTCCAGGACAGCCAGAGCTATACAGAGAAACCCTGTCTCGAAAAACAAAACAAAACA
AACAAACAAACAAAAAAATTGTGAGGAAAAATAAACATGTTTTCCTCTCCCCTCCACCTCTTTGTCTTTCATACCAAGCAAATCAAT
ACTTCTCCAATAATGAACTTTTCAGATTGCATCATGTGCACTCAAAGTTTGCTTGGCCACTGACAGAACAAGGTGTGAAGAATTCAC
ACCTGTGGCAAGCCGAGGGGCTGCCATCCTTCCAGCTGAGCCTTGATAACCACTGGCTACATTCTATCTGATTTCTCTGCCAACATT
TTTTCTCTGCAGCGTTTATGTAAGTGTAGACTTGTTTGGTGGTTTCTGTGTTTTCTGTTCAGTTTCTCCAACCAGGGTAAATAAATG
TCCAAGTTCTAAGCTTAAACACAAAACTGGGCTTAGCCCTGGGTCTGTGCCTGCAGTGCCCTGCAGGGTGCAGCACATCTTGGGGCC
CACTCCCCTCCTCCCTGCTTTGAAACTGCCCAGCCTTCCTCTCTGACTTTATTTAAGGCCGCCATCATTTCTACTAAATTAAAATGT
TATACTTTAGTCTTGTAGCTTTTTTTTTTTTTTTTGTCAAACACATTTTGTTGTTTTTTAAGTTGACACTCCTAAAAAGAAACAACT
CAAAAGCTCTAAAATGCTGTTCCCCAAATGTTTCAACAACTTTCCAATGGATTGAAAGTTAAACCTCCTACACTCTGGTTTGATTGG
CACTGCCTGGGAAGCACACTGCGGCAACTGCCTGGCCTGCTTGATCAGGAAGTGCTGAGATAGACATCTGCTCCAGTTCTAAAATAA
CCCATGACCTTCAGCCCGTCACCAAACTGCTCCAAATTCGATTTTCCTCATCCAAAATCAGATGTGTCAGCCCTGTTCCAGGTCGGT
GGCAGTGCTGTGTAAAAAGACATTCACTATCTTTTTTAGATTGGATGGTAAACCATACTTCAATTAAGTGATTTGATTTGAGGTTTC
AAATGTAATAAGAAAATCAAATAGTGATGATTCTGCGTGTTTCAGGTTCTCAGTTCCCAGAACACTGTCGCTGGTCTCCAGAGACCA
AATCCAAGGGTGTCCTCTGCATTTTAGAGAATCAGTATCATAATTCCTGGCAGGCTTTGACATGTTCTCACACTCCAGAAAGGAGTT
CTCTTAAATGGGTATTGCATGTGGAGAAATAAAATCACGTTGGATTCCTTATGTAGTAGAAGAATAAAGGAATTGAAAGTAAGGGAG
CGACTTGCTTGTTTTTAATCTCAGCCCCTGAAGTGTGGCGAATGTCTCCACCACTGAGCACGTGAGCAGACCTGTGGGATCCTTGGG
ACCTGAACCTGACGGGGTTGTCCGGGAGGCTCAAGTGTGGAGTCTGGCAGAGCGCTTTACCTTGGGAGGCCTGTGAGCGCTTGCAGA
CAAGCCTGCAAAGTTCACTCACAAGCTCCTCGCTCCCCCACCCCACCATATTTCTGGAAACATGCCAACGGCTTCCTTGCCAGTGGT
CCCGGGGGACTTAAAATGACTTCTTTCTGTCCAAGGTAGTGTATTTGTTGTGACATCTACTTTTTACTCAAAACAGTATGTATTTGA
GGGCACTGAAACAAAGATACCATCTATAGTTGCTCCACCCAGAGCCTGATAATGGTGTTTTTATTAGTGTTGGTTGGCTATTTTATC
ATTTTACCAAAAATACAAGCACGGTTGATCCTGCAAAGAAATCTATTTCCCTCCCTCCCTTCCTTCCTCCTTTTCTCCCTCCTTCCT
GCTTTCTCCCTCCTCTCCCTCCTTCTCTCTCTCTCTCTCCCCCTTCCTTTCTTTCTCTCTCCCTCCCTTTCTTTCTTCCCCCTCCCT
CCCNNNNNNNNNNNNNNNNNNNNNCCCTCCCTCCCTGCCTCCCTCCCTCCCTCCCTCCCTCCCTGACTCCATCCTGGTCCTCCAGCC
TTTCTCTTCCTACTTCCCCTTTTCCTATTCCTTTTCCCCCCTTTCCCTTCACTTACCTGGTCTCTTGACTACAGTCATCCCAGATTG
CCAGCAGATGACTGCTGAATATGGCCAGATAACTCCTGTGGGAGAGAATGGCACTTGCTGGCAGCTTGGCCTGGTGTCCTGAGTTTT
GTTTTTTTGTTTTTTCCATCTCCAGGGTGTCCTCTCCAACATCTCGTCCATCACCGACCTCGGTGGCTTTGACCCAGTGTGGCTTTT
CCTTGTGGTGGGAGGAGTGATGTTCATTCTGGGGTTTGCAGGGTGCATCGGAGCACTTCGGGAAAACACCTTTCTTCTCAAGTTTGT
AAGTATTGCAGACTCTGCTCTCAGCCTGAGATCTAGGCTCTTGTTTTACACCAAGACGACAGTCTTAGCTGCAGCCAGGATAGTAAG
CGTGCCAAAGACCTGGGCAAAACATGCAGCCTAGTCCACTTTCATTTCAAGAGCGCTCCTCAGTCCTGCCCGCTCTTCCCTTGCTCT
AGACATGCCTCCTAGGCAGTATACATACCACCTAGTCTTGCCATGCATGGAGACTTTGTACATATATGCAACGCATTTGACTTCAAT
CCCTAAAGTAGTTTTAGGAAGGAATGAGATGTTGGGGGCGGGGCGAGTAGTTTGTTGGATGTTGAATGTGCATGTGTTGGTGTGTTG
GTGGTGAATATTGTAAATAGAAAACCTAGGGTAACAAGGCACTAGCCCAGCTTCTATAGCCTGTGCAGCCTGCACAGTTTGAATAAA
CATGGCGATACAGTAAAGGTGAAAGACCTTCTTAGTTTCACCACCTGTATTTGTAAGGGGGGGGGGGACTTTAGCAGGAGGTATGAA
ATATTCAGAGCTCGTCCTAGAGAATGGGCAATGAAAGCCTTCCCTTAGGGCTCCTTTCCAACAGGACAGCGGCATGGGCCACATATC
AGTATCTGAAGTCAGAAGAATGGTCAATGTGAAATGCAAATACAGCTTCCGTGATGGCTGATGTCATCAAATGTAACTTCAGGTTGC
TTTCTGTATCTTTGGAAAGAGAGGACTTAGCTCACAGGGGCCCCGAACCTGTCAGTTTCTGCCAGATACTTGGTATGAACTGAGTTT
GGCCTCTTTGTGTGTGGAGGCTGGCATTTTAATGATTGCTGTAGGAAAAGTATTCACTCTCCTCTCCAAGAACAGGGAAAGGATCAG
AAGGACCCACTGTCCCATGATGGGGGAGGACAGGGGAATTTCTAGAGAACAACTTTGTTGGCTATGCCTCTTATTAGAGCCATTCGC
ACCAGAGCCCATCCTAGAACAAGTCTGAGCAGCCACACGGTGTGTGATTCTCCGACCCCACCATGTCTTCTAGAATGTTCTCTGGCG
TTTTTTATGAGCATCTCTAAGAAGATACTCCCATGCCCATTCTAGGGTAACCCATTTCCAATTGGCCCAGCTCTGGCTCCCTGAGAA
GCCTTGGAGGCTGTAATCCTAAATCTTTTCATGTGGCTCACACCTATATGGAAGACTGGGGTGGGGTCATTGCTCTCAGATAGCACC
CTTTTCCTGTGCATGGGAGTAGCTAGTTTCCCATGTGTAGAGCTGCCTTGATCTTCCAGATTTGCCCCTTTCTACACAAGCTTGTGT
GATCTGTCCCTTCTTCCCTTCTCCCTCTTCGCTGAGTCAAAGACAGATGGAATCCCTCAGAAGGATATTTGTCCTTGATCATGACCT
GCCAGTCTTATGAGGCATCTCGAAGTGGGGACTTGGTTCCACTCAGCTTTGCTGTGACCCATTATCTGCCGTGTGACCTAACACTTT
GAAACCTCCATGCTTCCAAATACGAGGTCCCTTTAATATAGCTTTCTTGAACATGGTTTTACTTATTATAGTAATGCCCTTCCTAAC
GTTTCAGATTTGAAAAGAATCATGTTCTTAGCTTTCTATCTTGGAGACTATTTAAATGGCCTTTCTTTATTTAGAACTAACAATAGA
ACCTGGCCTAGAAGTCTCTCTGAATGGGGGCTGTGAGGTAAGGAGGAAACAATGTCACCGTGCCTCCAAAAAACCTCAAAGATCAGA
TTGTCCTAAGACAAGGAGAGTTTCTGGGGGAAATCCAGGAGGCACGACTGTGGAATAGAAGTGGCATTTCCTAGCTGACCGATAGCA
TTGAACAAAGCTTTGGGTCACTTTCTGCACCTTTGGAAATGGGGGTGTTAGTTCACAGAGCCGGGCATGTAGGCCTCTGCCAAATAT
TTGATATTGAGTTTGAACTCAATATTTGAGATGTATATCATGTAACTTGAGGGAATTTCCGAGGGCACCTGGGAAGTGATCGTCTCT
ATCCTTCATGGAGGTGAGAGCAGAAATCTACAGTCTCTGTGTGACTGCCCAGCTGTTTCACGTACATGCATGCCCACCCCCGTCCAG
GGTCTATTCTTTCTGTGGTGTGAGGACCCTATGTGTCCATGTTTGGGGAGGTACCCAGTACAGGTAAAGGGGTTGCAGGGGGCACAA
ATACTGGTGTGCTCCTTCTTTTCTAAGAAAACGTAATTTGAAAAGAATCGTAATTTGAAATTATTGATACCACTCATAATTAAGAAC
TTAACATTTTATTTACAGCTGCAAATAATTGGTACAGTCAGTAAACAAGAACTTTACATTTTATGTCCTCACTGAGGAAAGATAAAC
AGGAGCCCTACGGGAGCTTGGAAGTGGAGTTTGTACCTCAGCCTCAGCATCAGCATCCTAGACTGTCAGGCACTGGTGTGGTCCTTC
TGACTCTCTCCCCTCACTCCCCCACAAAGACGCATCCATCACTCAGTGCCACGTGAATGGCCACACCCCACTGTCCACCCACCATGG
CCCCCCATTTTTGCACCTCAAGATCACAAAACAGTTTGCAGAGGGTAAAGATGTGGGAGGAAGGAGATGTGGAAATGGTGGTGGGTG
CAGCCGGCAGCCAGGATGACTAAAATGAATCTCTTTGAAGTGTCTGGGTGGAAGGGAAATTTCCCCAGGGTTCTGAGCAGTTACCCC
AGGGCCTCATGCTCTGAAGCCGTATACCTTGTTATCTGCCTTAGCCAGAAGTCCCTTCTGATTGCACTGTGGTGTTATTTAAAGTGT
TTCCTGGTCTTGGTGTTTCACAGGCAGGAAGAAAGAGAGCCCAGAGTTTCATTTCCATGATTGGAAATGGTTTTCTTGCCTCAGTCA
CAGCCCCTCCCACTTCCCCTGCTGACCTCTTTAAGATGAGCGGATCTATTTCACAGGGCTGGGAGGTGGAGGGAGGTGTTAAAACAG
TGACTCCAACATCAACACGATAATGTAGTGTGTGTGTGTAGTGTGTGCGTGTAGTGTGTGTGGAGTCAAGCCACGGTTGCAAGGGAC
AGGTGATTATAAAACCCACGATAATCTACCTTTCAAGTTAGGAAAGAGGCTTCCTGGAGGTCAGGCAGAACATTGTTCACACGTCTG
GCAGTGCATTAATCACGAGCAATTCATTAGGCCTTAATGTGGATCTAATGAGAAATTCCTATTACTCAATGTTTGATTTGGGCTTTG
TTGGTTAATTATCAAATGGAACACCAGAGACTCCAAAATGTGATTATGAAGTGATCTTTAATTTTTAAAAGTCCTTTCTTGGGGTGT
AATATTCCATTAACAAACCTTAAGAATCAGAAGGCCCCAGTTTGGAGATGTGTAGCAACTGAATGTAATTACAGCCCCTTTCAGAGA
AAAGCCTCTCGCTTCCCTGTGATCTAAGGCCTTGTAACCCGTTCCCTGTTGTGTGTCTCTTTTGTCTTAGTTTTCTGTGTTCCTGGG
GATTATTTTCTTCCTGGAACTCACTGCTGGGGTGTTGGCATTTGTTTTCAAAGACTGGATCAAAGACCAGCTGTATTTCTTTATTAA
CAACAACATCAGAGCCTACAGAGATGACATTGATCTACAGAACCTCATAGACTTCACCCAGGAATATGTAAGTTCCAAGCAGGAGCA
TCTTTTCTCCCATAATGCCTTGTGTGCCTTTGCCAGCTGGAGACGGCCAACTGACGTTTCTGCCCAGTTTCTGGCTGTTTGCTTGTT
GGTTTGTTTCCTTTTCCATGTTCCACCGCTCCTGTTTCATCTCAAGGAGACATGTTCCGAGTTCACTTGCTCCAACTTGTGTTAAAT
TAGAGGAAGATATAATTCTTAACTAAAAAGGCGATTACCGCACCGTCCCGTTGTCAGCTTTTGAAGAAAATTGCTTTTATATATGTT
TAGAGTTTTATGTGATTAAATAAATTAAACGAGTTTTCCTTGCTCCTTCCCCTGTGCTATCAATAATAAAATTATCAGGGGGATTTG
TTTNNNNNNNNNNNNNNNNNNNNGAACTCACTGCTGGGGGTGTTGGCATTTGTTTTCAAAGACTGGATCAAAGGACCAGCTGTATTT
CTTTATTACAACAACATCAGAAGCCTACAGAGATGACATTGATCTACAGAACCTCATAGACTTCACCCAGGAATATGTAAGTTCCAA
GCAGGAGCATCTTTTCTCCCATAATGCCTTGTGTGCCTTTGCCAGCTGGAGACGGCCAACTGACGTTTCTGCCCAGTTTCTGGCTGT
TTGCTTGTTGGTTTGTTTCCTTTTCCATGTTCCACCGCTCCTGTTTCATCTCAAGGAGACATGTTCCGAGTTCACTTGTTCCAACTT
GTGTTAAATTAGAGAAAGATATAACTCTTAACTAAAAAGGCGATTACCGCACCGTCCCGTTGTCAGCTTTTGAAGAAAATTGCTTTT
ATATATGTTTAGAGTTTTATGTGATTAAATAAATTAAACGAGTTTTCCTTGCTCCTTCCCCTGTGCTCTCAATAATAAAATTATCAG
GGGATTTGTTTGTAAAGTTCCTCAGATATGCTCATCCAGATACCACATGCATGTAGTCCGAACCAAAAGTTCAGAGATTCGTAAAAC
CTGCTGCCCTCTAACAAGTGAAAAAGAAGCAGACCCCGCCTGCCACACAGGTGTCTGAACGTGACTGTGTGACTGACTGTTCCCATA
CTGTAAACAGGCAGCTGTCATGTCACTCAAAATGCTCGAGGAGCCAAGCGACACTGCCTCATTGACCATGGACTGACAGAAACTTGA
ACTCTTTCCCAAGAGTAAAAGGAGAATAAAACTGAGTTACTATCTAGTACGTATGGCTGTTGGGACAGTGGTCTCACCTTAAGCCCT
AGATGTTACCAGACTAAAGATTAGCAGGTCCTCACACACCTGCCCTGGAAAAAAATGAGTAAGCCGAGCGACCTTGGTTCCATGGTA
TCCATGGCTTTAATATTTAGAACAAGATAGGAGACTACACAGAGGCTCTGTGGACCAAGGTCTAGCTCTGAGTTGGGTAGGTTGCTC
TGGGATCACATTTACCTTCAGTCAAGAACGCCACACTGCATCAGCATTGAGTGGGTGTTGAGGTAGGCCATTCTTCTCTAGGTGCTC
AGCACTGGGCTGAGGTCTTAGTTTCACTCAGTGGCTTCTATCTAGAGCAAGCATCCTCAGGTGGTGTGAAATTGAGCCCTGTCTCAC
GTGCTCAGGCGATTTCCCTGTGCTGTTAGGTCATAATGGATGCAAGCTCTCAGCTTCCCGGGTATTCTCCCTGACTAGCTGGCATGA
CAGAGCCGGCGAGGCTTTACAGGAAACCTGTGGTGTCCAGCCCAAGCCCAGAGGAGCAGCTGAGCCTCCAGAACCTGTGTTTGCACG
CGTTCCTAAGAGCTGCCTGCTCTACTTAGCAGCACTGCTCTTTCATGTGATTCTGGTCTTCTGTTCTGGAGTTAAAAACATTTTGAT
GTAGCTGTTGGTCCTGAAAGCCTTAAAATGGCCACAAATTGGTTTTATTTAACTTAAGAAGCCAAGATGATCCAGTACAGAAGGAGG
CCATAGTCAGTCCCCATTTAAGATTTGAGTTGAACTGAATTTAAGAACTGAACTATAGGCTGGAGAGATGGTTCCTTGGTTAAGAGC
ACTGACTGCTCTTCCAGAGGTCTTGAGTTCAATTCCGAGCAACCACCTGGTGGCTCACAACCATCTGTAATGGGGTCCAATGCCCTC
TTCTGGTGTGTCTGAAGGCAGCTACAGTGTACTCACATACATAAAATAAAAAAAATAAATCTTTTTAATCTTTTAAAAAGAACTGAA
TTGAGCATAAAAATAAGTTCTTTTTAAAAATTTAGTGTAGAGATGAATATAATGAATAGTTAGGCCTCTAGTATTATAGAGAGGTGG
GTCTGCCTCATCCGTGTTTGACCACTGTAGCAATGGACATCAGCCCTGACGTTCACTGTCCTGTGAAGTGGCTACTGTTCTACTGTG
TTACCCACAGACCAGACCTCATCAAAGTGTCTACTGTTTACCTGATGCCACTTAAACACAGTGAAACTGTTTACGAACCATTAAAAT
AAGATCAAAACTTAAGCACAACCACAGTTGCCTCGTTATACGTTACCGTTGTTGCTAGTGTCTTGTTTTTCCTTCTAGAATGTTCTG
GTTTATCCTAGAACGTGACCGAGCCAAATTTATCGTGAGCCCTGCTGTACAATGTCCCATGCCAAACCATCTCTAGGAACCTTCAGC
CTACATCCCTTTGGAAGGCTTTCCAGGGCCCGTTTAGGTTAATTTTAACTTAAAACTAAATGTTGAGCCCCCGGACCTGTCTGGCAG
GGGATGGGTGTCATGGGGGTGGGGTGCAGGTGGCTGGTCACCCCACCTGAGATGTCCTTTCTAACTCTCTCCTGTTTGCTCTAGTGG
CAGTGCTGTGGGGCTTTTGGAGCTGATGATTGGAACCTAAATATTTACTTCAATTGCACAGATTCCAATGCAAGCCGAGAGCGATGC
GGTGTGCCATTTTCCTGCTGCACTAAAGACCCCGCGGTAAGAGCGACTGCTCACTGCTGCCTGCCCGCCGTGTGCCCCGTGTGACGG
CAGAGCCATTTCTGTCTCAAAACTCTCTTCTCACAAGCTCAGACCTGACTGTAGGTCCTAGAGGAGGCTGATACGCTCCAGTGTTCC
TAGTCAGGACAGGATGGCAGGAATTAATTTAGCCAGTGCTTGTTGTCTCCTATTCTGAGCCCTGGTGTGTTGTCTCACGGGAGGTGA
GAAAGGGAAGTCCCTTTCTGGGGAAGAAAATAAAACCCACCAAGGCCTATGGAGTACAAATGATTTCTCCCAAATAACAGGGCTGAC
AAGGGACAGAGCCGGGTGACTGTGTCTCTTGTCTCTCCTTATAATGTATGAGCCACCTCTCTGCAGTAGCTTGGATTTAGTAAGCCT
TTTCTTCTCCTCTGGTTTCTGGGAAGGGACTCAGGAGTAAAGAAGGCAGATGAGGATGGGGCGCTGCTTGAGGACCCACAGTGGCCC
GGAGGGTGGCATGGGGGTGGCTGCAGCGGCTGCCTTTTGCGGTTTTACTTTAGAAAGAAAACACGGAGTGCGTCCACAAGCTACTCC
TCTTTCCTACACTTCACATCGTGTGCATGGCTCACAGAATGAAAATGTGAACCGTCTGTATTGTAAGTGGAAAGCCAGAACTGAGTG
GTAGTGTGCCCCGTGTGGGGAGTGGCTACTGCCCTTCTGGGACAAGCCTGCCCATTGAGCACAGTGGCGCACAGTGGGTGCAGCGTC
CCACAGGCTCCCCCTCCTGCAAGGTTGGGGCTATGCGGCTCTCTCGCGAGGCTTGGGTTTTAGGATGTTGAGCTGCAGGCTGTGCAG
GCTGCTCTTCTGGAGGGCGCAACAGCCTGGGCTGAGCGTCACTGAGAACTGTGTCATCCTGCCTGTGACTCCTTCCTAAACTTCCTA
GCTTGCCTTCCTCCTCTGAGGCGGCTCAAGCTGCAGCAGGATATCCAGCCTGGAAGCCATGGTACCCAGCATAATGTGCTTGCTGGT
GGGGAATGTTTTACTATATACTTTTGAAGCTGCTCTAGTAAAAGCATGGCATTTAATAATAATAATTGTTAAATAATATTTGATAAT
TAATTTAATAATAATGTTTAATACTGTGCTCCAGTGCCCATCTTAGCTGGGCTTTCTGCTGAACAAGAATGAGTTTCAGTTTTTCTC
TTCCTTTCTTCCCCAGGAAGATGTCATCAACACTCAGTGTGGCTATGATGCCAGGCAGAAACCAGTAAGTGAAACCCGCCCCATGTC
TTAGCACATACGGATTTTTTGGAGGAGCGTTTGCGCATGCGTAAGGCTGTATCTATGACATTCTGCTCTCTTCCCTTGTAAAGGAAG
TTGACCAACAGATTGTAATCTACACAAAAGGCTGTGTGCCCCAGTTTGAGAAGTGGCTACAGGACAATTTAACCATCGTGGCTGGTA
TTTTCATAGGCATTGCATTGCTACAGGTAAGATGTGGGTCCTCCGTGCTAGCTCAGACGGTGGTGCCCAGAAAGACCTCCCCAGTCC
AAGATCTTTGCTTTGTCTCCTCGATGCCTTAGCTTCTGAGACACACTGTAGGTCCTAGAATCAGACTGGAACTATTGGGTTTGTGTT
TATTGAAGGCCTGAGTCTAGAGACAGAAATACCTTAGGAAGTCTTGCCTCCTTTCACCACAGCGGTGTTGATGGGTCCCAGGTTGTT
ATCTGGCATCCCATTGTTTCAAGGATTCTTAAAGGATTGTGTTGTGACACTTGGGGCAGACAAGGCTGCTCAGAGTGGGGACCCCAG
CAGCAAGGCCAAGCTCTGTGGAGTTGCACAAGGACGTGCGATATCTTTTGTCTCTCCAGACCAGCTCACCCAAGTGCTGGTGAGGAC
AGTCCCAGGAGCTGACTCTTCCTGTTCATAAGATGCTTTCTGTGGAGGGCCACGGATGGGTACATGGTTGAAGGAAATTGAAGAGGT
CTTAAGTAAATAGGAAGGCAAGCTGTGTCACAGATTATAAACTCAGTGTCGCTCAAGTGGCATCAGCAACTCATGGTGATCTAGAGA
TTCTGTGCTCTCCGTCTCTGTTCCAGCCTCCTCATTTAGTCATTGACTAGCTGGGCCTGAGTTCACAGGGAAAAATAAGAGCCCAGA
GTAGTCAAACACACTGAAAGAGAATGGCAGGATGAAGAATTCTGGTTTCCCAGTTAAAGCATACTACAAAGTAACAGGGTTCAAGGC
AGGCTTGTACGGTTGTAAAGTTAGCCATTTTGATAGATGAGATCGGATTACAGAGACAACTTTTGTGAGCTTTGTTTTGATAAGGGA
ATTGAAACTTTTCAATGGGGAAGGAAAAAAGAACTTTCAAGGATGAAGGAAAAACAAACAAAACAAAAAAAAAAACAAAAAACAGCT
TAGTGGTTCAGAGCACTGGCTGTTCTTCCAGAGGACCAGGGTTCAATTCCCAACCCCTACATGATAATTAACAACTGTCTGTGACTG
TAGTTCCAAGGGACCTGATGCCCTCTTCTGGCCCCCATCCACACTTTATGCATGTGGTGCACAGTCATGCCTGTAGGCAAAACACCT
ATACACATTTCAAAACAGGGGGAAATTTTTTTAAAAAGTTTTTTGACAGATGGTGCAAGAATACTGATTTTTCTGTCTAGAAAAGAA
AGGTGTTTAGACATCTATCCCATACCATATATAAACATAAACATAAACTAGATCAAAGACCCAAATCTAAGTGTGGAGTTCTGTATG
CCTGTAATGTAAGCCATTAAAAAAGGTTGTAGCAGGATAGCAGGATCGTCACTTAAGAGTACACAGCCAGCCTGGCCTTCACACTTA
GACCCTGTCTTGGTGTCAAATAATTAAACAAAGCTCTCTCAATAAAAAGCCAAAGCTGTGGGCTGGAGAGATGGCTCAGTGGTTAAG
AGCACTGACTGCTCTTCTAGAGGTCCTGAGTTCCATTCCCAGCAACCACATGGTGGCTCACAACCATCTGTAATGGGATCTGATGCC
CTCTTCTGGTGTGTCTGAAGATAGTGATAGTGTACTCACATTCATAAAATAAATAAATATTTTTTTAAAAAATGCCAAGTTGTGACA
GTCTAGAAGAAAACATGGTATAAATTGGTGATTCCCTTTGTTTTGTCTTGCACAAAGAGTTTCAGGGGAAAAAAAAAACCATCAGAT
GAAGGAGAAATAATGGCACTTCATCAAAATATAAAACTGAGGTTACCCACAATAGGAGAAAGTATTTTTGAGGAGTTACGTGTGTGT
GTGTATGCATGTATACATAATATATATATATATATATATATATATATATATATATATATATATATATATCTTATAGGAAGCAAGAAA
AAAGGCTAGCATGGGCTATCTACCAAGGTCCGGTTTAAAAAATGAACAGAGTACTTAGACATTTCTCCAGCAGAGACAGGAATGTCC
CATGAAAAGATACAGCATCACTTGCCATCAGAGAGCTCGCCAGAGCTGCAGTGAGGTGACCATTGCACACGTACACCCACTCGGACA
GCGGGTGTTCACGAGGATGGAGGATGGATCACCCTCAGCTTGCTAGAGTGGGGGAACAGAAAGGGCCCGGCTCTTTGGCAGATAACA
TAGTTACCATGTGACCTTACAGTTTTTCCACAAATGCCAAAGGAATGAAAAGCGACACTTACCAAAACATTGGCACACACGTGCACA
TAGCAACAAGAACTGGAGACGCTGAAGAATCCATCCATGGATTTTTAAAGACTGTAGAACGGCCTTACGGTAGGGTCGGTAGTCAGC
CATCAAAAATGCAGGCATGGGCTGTGCTGCGCATGCCTGCAGTCTCAGCCCCAGGGAGGCAGAAGCAGAGGCAGCAGAAGAACTTAG
ACTGTACAGGAAGGCTCTGCCTCAGAAGCAAGCAGACACCACAAATAGAACAGACTGGCCAGGTGTTTGTTTGCAAGTCACCACAGT
TACTTTCTTTAGCTGCTTTTGTATAGAACCCAGAGCTTGTGTCCCTTGCTGGCGTCCTGCACCACATCTCCCCCACTTCCATCTGAG
TCAGAACTCTGAATCAGCCACTCACCGCTTCTTCACAGTGCCCTGAGGGTGCCCACTTCTCAGCACAGCCTGAGTCCCTGACTCAGA
AAAATCACCCACCTAGTCAGCCTGGAGCTCTGCCTCCCCCCCAACCCCCACTGTGACCTTGCTGATGGGGAGAGGGAGGAAGCTGCA
GAGCCAGTGAAGTGATGGAGCCTTCCCTGAAGGTCACAGACCCAGCCTCAGCAGGGAGAACAGGGTGCTGAATGGATCAGTTGGTAC
CCAAGCCAGAAAAGCTGGTCACCAACAAAAATGTTCCCTGCATGTGTGATTTTATTAAGGCTTATTACCTGGCAGAGCCCATGAGAT
CCATACAAGGAGTGGGTTGTGACCCACCTGTGCAAGATAAGAGCCCAGCTGGGCTTCAGCCTGACATTTCCACAGACACGTGTGGTT
TCCCTGGTCATGAACACAGCGCATGCGTGTTAAGGTTACCTCAGCTCTCCTTATCTTGTGTAAAATGTTCACTAAAATTCTTTGATT
TGTACCCTGTAAATAAAGAAAACTCATAGTTGGGTCCTGAAGGGTTGTAGCGCTGTGATTGAAACCTTGCAGAGGAAAGAAAGAGAG
GTTTGTTCAGACCCGTGGTGGGGGAAGGCATAGAGGTCATAGCAGCTCGGAGACAGATAACTCCTTTGACAGCTGTCCCTAGCAACA
CACACGTCTCAAATGTCCTACAACCTCCCAAAACATTGCCACTCACTGGAGACTGGGCGTGCACACCATGAGATTGCGTGGGCTTTT
CACATTTGAGCGGTCACCGTAGCAATGGACGAACCCAGAGATGGTTTTGCTCCTTGAGAGAAGTCACAGGAACCACATGTTGTCCGA
AGGCATTTTCATGAAATGTGCAGATCGGGCAGATCCATAGAGGGAGCAGGGAGGTGAGAGCTTTCTAGGCCATAGGGAGGGTTGGAA
ATGACTTCTCGTGGGATAGGGTTAGCTTAGCGGGGTGAGCAGCTCCTGCATTTAGTATTGAAAATATAAAGTCATGCTAAATCTCTG
GATTTTTTATTTTCTATCTCTCTTTCGCCACTCTTCCCCCCCCCCCAGCCTTTTTTTAAAGACAGGTCTCATGTATCCCAAGCTGGC
CTCAAACTTGCTCTGTAGCCAAGGATGGCTTTTTACACTCCTGCTTCTTCCCACCCAAGGGTCGGGATTATAGATGTGTACCACCAT
GCCTGGCTTTCTATGGCGCTGGGGGGTAGAACCCAAGGTTTTATGTATGTGAGGCAAACGCTACCGACTGAGCTACATCTCCAGCCC
CCAAATATCCATTTAAGAATCAGAGGGGGTGGACCTAGTGGCCAAGGAAAAGCCAATAGCATTTGAATTCTGCTGTCTAACTTGGTT
TCCTTTCCCCCTTTTTTTTTTAGATTTTTGGGATATCCCTGGCACAGAATTTGGTGAGTGACATTGAAGCTGTCAGGGCTAGCTGGT
AGACCCCTGCGACCACTGCTGTCAGACACTGGACTGATCCCCCCTCACTGACTGACCCTCTGTGCGCCGGCCGGCTGATGGCGGGCT
GTAGAGGCCTGGTCACAGGCCTACAGTCTCACCTGATGGAGCTGCCAAGAACTTAACTTTCTGTGGGGGTAGAACTGGGAAATGCTG
CTCACAGACGGAACTTGGAAAAAGAAAAACAAAACAAGTGCGCAACTTGCTGAGTCAACGTGCAGTGAATCTCTACTGTAGCCGTGG
ATTGGTGGTCGGATGGATGCTACCAGAAGGGGGAAGGGGGGAGGTTGGGCACGGCATGTACTTGAATTTTTGGAGAATACAGTGCTG
TACACATCTTGGCTAAAACTTGGGCCCTTAGCCGTCAGGGCCAGGTGTCTTGAAATCTCTCAAACCCTTTGCATCTGTCCTCCAGAG
AAGAAACCAGCTGCCCCCTGTGTTCAGATTCTGAGAGAGGTGGAGCGGGTCACGATACAGCATGCGCCCTTCATCCTGAGTTTGCCC
TTAAGAGAACTGGGCGTTTGCAGTAAACTAGGGTGCCAGAGGCTGAGCTCCACATTTCCACAGGACTGCTCTTTCCTCTGAGTGGCC
AGCCCGAGCCTGAGCTCTGTCAATGACATCCAAGGAGAAAATGAGGTTAATGAGAGACATTAATTAAACACTCCCTCACCCCACCGC
ACCAAACCAGTTGGGTTCTTCTGATATTCTGGAATACTCTGGGCTATGTTTTATGTTTTATTTCTTTTTTAATCGGTTGTTATTTTG
GTTCTTTTTTTTTTTCTTTCTTTCTTTTTTCTTTTTGTCTCCCAAAGGAGGCTATGACACAGTTCTCTTGGTTTCTTATTCAGCTTG
AATAGTAAGTTTTTGTAGACTTGTCACCTGCATGTACTGTAACCCCACCCCCACCCCCAAATCCACCATGTTCTTTTCAGAGAGACA
TTTGAGCGTGTTCCTTAGTGTTTATGTCCTAGGTATCCAGCCTTGAGTGGTTGTGGAGTGTTGTCCTAGCCGCATCTTTGTATGCCC
CTCAGGCTCTCTGTCTCTCCTGGCATCCAATTCAGCTTCTGTCCATTCGCCATTGAGTGGTAGAAAAGCCCAGCTCCAAAGCAGGGT
TGACACAGTCAAAAAGCATTTCATGAGGTTTTTCTTTCTTTTCTTTTTCCTCCCTTTTTGCAACTTTAGTTTTGTCGTATATTAAAT
AAAAAACCAAGCCTAGTATAGTGCATGTGCTCGCTGAGGTGACCTCGGGGTGGTTACAAAGCACAAATATTTCAGGGGCTGCGCCGA
AGCCCAGCCAGGGTTCAGTGACGCTAGGTGGAAAACCACCTTCAGGTTCTTCTCCTGCCTCGTGCCCACTGGGAGGGTGGACTTTAG
CCCCAGATCCCCCCACCCCCAGCTTGTGATAACCCCTGGATGTTGCTTTCACGGGGAGCAGATTTAGTTTGTGGGTTTGCTGGGAAG
GCCCCAGCCTCTGCGAAAAGCAGCTCCCCATTGGCCAGCCAGCAGTCTGCGTCATGCAGCCCGGTTTTCCATTTCCGCAGCACGTGT
GCGTTGCCGTGGCTGATGGGCGCTGTCTGCTCAGAGGGAGGTCAGGCACTCCTTGGGTTTACGTGGCTCTGCATGTTTGTATCGGTG
TCTGTTTTTAGGTAGAGATAGTCATTCCATACGTGGATGCGAGGTCACGTGACTTCACCCCTGTCCATACCACAGCCGTCGCTTGTG
ATGTCCTTCAACATGGACACTCGTGCTCTCGGATTGGCACTTCTGTCTTCTTTATTACCTCATTCTCCAGTGAACTGCATCTGGCCA
ATTGATTCAGAATCAGACAGGATCCCCACCCCGTGGCACATCCAGTGAAAGCCAAAACACAAGCCCAAGCGAGTTTTAAATTTTAAT
CACCCTTTATTTTTTACACTCGAGAGTGATTGTAATAAAAGCTGTCATTAATAAACTCGGTTCTACCT
MOUSE mRNA SEQUENCE: mR28-002.1 (Seq ID No: 50)
CGCGGCATGAGCCCCGCGTGCCCCACTGCTCGCCGGGCTGCTCCGAGCCGGGGTGCTCCGGGGCTCCAAACTCGGGCTGGCGGGGCA
AGTGTCTTCATGAACCCAGAGGATGTCCGGGAAGCACTACAAGGGTCCTGAAGTCAGTTGTTGCATCAAATACTTCATTTTTGGCTT
CAATGTCATATTTTGGTTTTTGGGAATAACGTTTCTTGGAATCGGACTGTGGGCGTGGAATGAAAAAGGTGTCCTCTCCAACATCTC
GTCCATCACCGACCTCGGTGGCTTTGACCCAGTGTGGCTTTTCCTTGTGGTGGGAGGAGTGATGTTCATTCTGGGGTTTGCAGGGTG
CATCGGAGCACTTCGGGAAAACACCTTTCTTCTCAAGTTTTTTTCTGTGTTCCTGGGGATTATTTTCTTCCTGGAACTCACTGCTGG
GGTGTTGGCATTTGTTTTCAAAGACTGGATCAAAGACCAGCTGTATTTCTTTATTAACAACAACATCAGAGCCTACAGAGATGACAT
TGATCTACAGAACCTCATAGACTTCACCCAGGAATATTGGCAGTGCTGTGGGGCTTTTGGAGCTGATGATTGGAACCTAAATATTTA
CTTCAATTGCACAGATTCCAATGCAAGCCGAGAGCGATGCGGTGTGCCATTTTCCTGCTGCACTAAAGACCCCGCGGAAGATGTCAT
CAACACTCAGTGTGGCTATGATGCCAGGCAGAAACCAGAAGTTGACCAACAGATTGTAATCTACACAAAAGGCTGTGTGCCCCAGTT
TGAGAAGTGGCTACAGGACAATTTAACCATCGTGGCTGGTATTTTCATAGGCATTGCATTGCTACAGATTTTTGGGATATGCCTGGC
ACAGAATTTGGTGAGTGACATTGAAGCTGTCAGGGCTAGCTGGTAGACCCCTGCGACCACTGCTGTCAGACACTGGACTGATCCCCC
CTCACTGACTGACCCTCTGTGCGCCGGCCGGCTGATGGCGGGCTGTAGAGGCCTGGTCACAGGCCTACAGTCTCACCTGATGGAGCT
GCCAAGAACTTAACTTTCTGTGGGGGTAGAACTGGGAAATGCTGCTCACAGACGGAACTTGGAAAAAGAAAAACAAAACAAGTGCGC
AACTTGCTGAGTCAACGTGCAGTGAATCTCTACTGTAGCCGTGGATTGGTGGTCGGATGGATGCTACCAGAAGGGGGAAGGGGGGAG
GTTGGGCACGGCATGTACTTGAATTTTTGGAGAATACAGTGCTGTACACATCTTGGCTAAAACTTGGGCCCTTAGCCGTCAGGGCCA
GGTGTCTTGAAATCTCTCAAACCCTTTGCATCTGTCCTCCAGAGAAGAAACCAGCTGCCCCCTGTGTTCAGATTCTGAGAGAGGTGG
AGCGGGTCACGATACAGCATGCGCCCTTCATCCTGAGTTTGCCCTTAAGAGAACTGGGCGTTTGCAGTAAACTAGGGTGCCAGAGCC
TGAGCTCCACATTTCCACAGGACTGCTCTTTCCTCTGAGTGGCCAGCCCGAGCCTGAGCTCTGTCAATGACATCCAAGGAGAAAATG
AGGTTAATGAGAGACATTAATTAAACACTCCCTCACCCCACCGCACCAAACCAGTTGGGTTCTTCTGATATTCTGGAATACTCTGGG
CTATGTTTTATGTTTTATTTCTTTTTTAATCGGTTGTTATTTTGGTTCTTTTTTTTTTTCTTTCTTTCTTTTTTCTTTTTGTCTCCC
AAAGGAGGCTATGACACAGTTCTCTTGGTTTCTTATTCAGCTTGAATAGTAAGTTTTTGTAGACTTGTCACCTGCATGTACTGTAAC
CCCACCCCCACCCCCAAATCCACCATGTTCTTTTCAGAGAGACATTTGAGCGTGTTCCTTAGTGTTTATGTCCTAGGTATCCAGCCT
TGAGTGGTTGTGGAGTGTTGTCCTAGCCGCATCTTTGTATGCCCCTCAGGCTCTCTGTCTCTCCTGGCATCCAATTCAGCTTCTGTC
CATTCGCCATTGAGTGGTAGAAAAGCCCAGCTCCAAAGCAGGGTTGACACAGTCAAAAAGCATTTCATGAGGTTTTTCTTTCTTTTC
TTTTTCCTCCCTTTTTGCAACTTTAGTTTTGTCGTATATTAAATAAAAAACCAAGCCTAGTATAGTGCATGTGCTCGCTGAGGTGAC
CTCGGGGTGGTTACAAAGCACAAATATTTGAGGGGCTGCGCCGAAGCCCAGCCAGGGTTCAGTGACGCTAGGTGGAAAACCACCTTC
AGGTTCTTCTCCTGCCTCGTGCCCACTGGGAGGGTGGACTTTAGCCCCAGATCCCCCCACCCCCAGCTTGTGATAACCCCTGGATGT
TGCTTTCACGGGGAGCAGATTTAGTTTGTGGGTTTGCTGGGAAGGCCCCAGCCTCTGCGAAAAGCAGCTCCCCATTGGCCAGCCAGC
AGTCTGCGTCATGCAGCCCGGTTTTCCATTTCCGCAGCACGTGTGCGTTGCCGTGGCTGATGGGCGCTGTCTGCTCAGAGGGAGGTC
AGGCACTCCTTGGGTTTACGTGGCTCTGCATGTTTGTATCGGTGTCTGTTTTTAGGTAGAGATAGTCATTCCATACGTGGATGCGAG
GTCACGTGACTTCACCCCTGTCCATACCACAGCCGTCGCTTGTGATGTCCTTCAACATGGACACTCGTGCTCTCGGATTGGCACTTC
TGTCTTCTTTATTACCTCATTCTCCAGTGAACTGCATCTGGCCAATTGATTCAGAATCAGACAGGATCCCCACCCCGTGGCACATCC
AGTGAAAGCCAAAACACAAGCCCAAGCGAGTTTTAAATTTTAATCACCCTTTATTTTTTACACTCGAGAGTGATTGTAATAAAAGCT
GTCATTAATAAACTCGGTTCTACCT
MOUSE PROTEIN SEQUENCE: mP28-002.1 (Seq ID No: 51)
MSGKHYKGPEVSCCIKYFIFGFNVIFWFLGITFLGIGLWAWNEKGVLSNISSITDLGGFDPVWLFLVVGGVMFILGFAGCIGALREN
TFLLKFFSVFLGIIFFLELTAGVLAFVFKDWIKDQLYFFINNNIRAYRDDIDLQNLIDFTQEYWQCCGAAGADDWNLNIYFNCTDSN
ASRERCGVPFSCCTKDPAEDVINTQCGYDARQKPEVDQQIVIYTKGCVPQFEKWLQDNLTIVAGIFIGIALLQIFGICLAQNLVSDI
EAVRASW*
MOUSE PANTHER CLASSIFICATIONS
FAMILY (SUBFAMILY)
TETRASPANIN-RELATED (TETRASPANIN TSPAN-5)
BIOLOGICAL PROCESS
Cell adhesion (2.29.00.00.00)
MOLECULAR FUNCTIONS
Cell adhesion molecule (1.05.00.00.00) > Other cell adhesion
molecule (1.05.99.00.00)
MOUSE GENE ONTOLOGY
No Gene Ontology
MOUSE PROTEIN DOMAINS (INTERPRO SIGNATURES)
IPR000301 (TMFOUR)
IPR000301 (TMFOUR)
IPR000301 (transmembrane4)
IPR000301 (transmembrane4)
IPR000301 (TM4 2)
IPR000301 (TM4 2)
HUMAN GENOMIC SEQUENCE: hD28-002 (Seq ID No: 52)
CGCACGGACGCGGGCGCTCGCTTTGTGTTCGGGGCTAGCGTCGGCGAGGCTTGAGCTTGCAGCGCGCGGCTTCCCTGCTTTCTCGCG
GCCACCCCGGCTCCGGCGGCCTCGGCGCGCGAGGGGCTGGAGGTGCGGGAGCCGCTCTCCGCCGGTCGGTCCCCGCGCGGCTGAGCC
CAGGCCGCCAGCGCCGCGGCCCCGTGCGGTGTCCCTGAGCTCCTGCTCCCCGCCGGGCTGCTCCGAGCAACGGTGCTTCGGAGCTCC
AAACTCGGGCTGCCGGGGCAAGTGTCTTCATGAACCCAGAGGATGTCCGGGAAGCACTACAAGGGTCCTGAAGTCAGTTGTTGCATC
AAATACTTCATATTTGGCTTCAATGTCATATTTTGGGTAAGTTGGAGCGCTCCTGGGATTCCAACTATTGTGGCCGATCGATTCGCG
ACGATTTCCCCCACGTTGTTCGTTGCCTTTACTTTTCGCAGCCCCGCAGGCGCTTGCCGGAGCAGCATAGAGGCATTCGCCTTCCTC
CTTTCCAGCTTGCGCGTTGGAGAGATAAACATTTAATCCTTTCGAGGAATGGGAAGAGGTGAAAAGAGAAATCGAGGCGGAAAGGGG
GCACCGAGGCCTTCTGGTGGCTTTTTGAGGAACGAATAGCAGGGATGCAGATAAAAGAAAGGGTTCGGCAGTTTGAGAAATGAGCAA
CCACACGAATTGGATTTGCTCTCGGTGGGATATAGATGTTGGGCAGAGCGTGGTGTCATAATAGGGAAGGCTAATCGGGCACGGCCG
ACCCTGAGGTCCACCTTCTAACAACGATCTGATTGGACGAGGGCTTGGCTCCGTGTTGCCGCGGCTGGTCTGTGCCATGCTCAGCGT
GTATCTTCTTGCACCATCTCGGGCTTTGTGTAGGATGCAGATGCCGTGGTATATGGTCCTGTAATTGCACGTAAATAACGTTTCCTG
CACGCTCTCCTCTTCAGCTGGAAAGCGCTCCCTGTGCGTAATGGACTGGTCAGTGGGGGTGCGGGGTAGAGGGATTTATTTGTATGT
ATATATCATCGTCAGGATAATAAGTCACCGTCAACATATATTTCAGTGTGTTCCTGCCCCACGTCCTCATTTTTAAAGGAGTGACAC
AGAGCGGGCCGTTTGGGGGAACCTTTGTAGAAATGCAAAGGAGAAGGTGTGCGAGGCATGTTTTATTGGGGAACCGAGAGAGCAAAT
GCTTTAACACAAGATTCAAATGATGCTCTGCTTGGGACTGCAATGTGTTGGGAACCTATTGTAATTAGGCTGGGACGCCTACTCTTT
TTGTTTGTTTGTTTGTTTTGATTCTTTTCCTTGATCTGGCAAACGTGTTCAGCAGCCAGACAAAGAGTTCTCCAGGACAAGGGGAAA
ACGTTGGTTCAAAGACCATGTTATTTTTCTTCCCCTTCTCATCCCATCTCATCCTCTTCCTCAGTGGGCACCCGCATTTCCATAGAA
AGTAAAGATGGTGGCTGTCTGGGTAGGGGGTTCTCTAAGTTGCTGTTCAGTGTGAGGTAAACAATGCTCCAACTGCAGGATCAATAA
TTATCCACAGCAGTCTGAACACGGAATGTATTTATTTGAATTCTCCTTTAATTTGGGCTGTTTGCTGTACGTATATACATCTGTGAG
AATAGTTGTCAATACGGCTCTTCATATTCTTTTTTTGTACCGCACTCTTACCAGGTTATTTTATTTTCTGTTTTCTATTATAATACC
TTGCATTGGTCATGTATGTTTTGTGAGGGGAAATGCAAGGGATTGGAGACCACTTTCCGTGTCAGCATGTCATCTGTCTTCAGGACT
AATAAAGATAATGAGGGGACACATGAATGGATGTCATATCCGATCATTCCACAGGCAGTTTTTTGCCAGTGTTGTGCATGATTCCCG
GGAGCAGTCACCCACCTCCCTGCAATTAAACACTGCTACAAGTACAGGAAGAAGGAATTATTTTCTTTCTTTGCCCCTTCTCCATTT
GTGAAAATGTTTAAAAGTGGTGGGTAGCCACGGTTTTGAGTAAGATATTCTGATTTGATTGGAACTTGAAACTTTGTGACTTGGAGA
GGCACGGACTACCGAACTGGGACTGGTTCTTTTTGGGGTAGTGAGTTCAGGCCTCTTGTTTCTACTTGAACATTTCTAAAAGTTGCA
CATTTTAGCTACCCAGCAACAAAAAGCACCCAGGGACTTTCTCTGCCGTTTCCTAGCAGGGGGTGGTAAGGAAATAAAAGGATTTGG
GTCTGTTTCCCTGACAGTTTGCTGTAATTCTGTGGAGTGCATAGAGACAAATTTAGCCCTTTCTATGGATTAGGTTGCAGCAAAACC
CCTGGAATTGTAGAGAGCTTCAAAGAATTGCAGAGTGCTTGCTTGGAGAGGTCCAGAACAGAGCCGCATGTTTTCTGATTGTGAAGG
TCTTCATAGGTGGGTGGGTGAGTATGGGAAGCCTGAAAGGAGACCTAGGCTATGGGACTAAACTAGCTGCCCCAGGCAGAATTGTTA
AGTGAGTGCCTTCCTTATGAGGGCCCTTCACTTACCCTGCCTCTTGCCTTCCCTTCCTCTCCTCTCTGTGCCCAAAGCCTTCCTCTC
AGATACTCCCTCATACCTTTCCTATCCTGCTCTCTGCCTCAAAATTACCCCATATTTAAATTTCCTGCTTTATTGTACCATGGTCTC
TAGTGATGATGAACAGAAAGGCAACCATATGGTTTTGGTTAAAGTAATTCAGACTAAGATGTTAGTGACTGAAAAGCTGGGGTCAGT
AGCTTGGCAGCTTGCTTCTGAAAAGTTACACACCTCTGCTCTCTACAGAGTCACCAACCTGTCTGTGATTGCCAAAAACAGCAGACT
CGGTGCTACTCCCATACAATTATGAAGAAGAGATTGCCAACAGCATGGTTTCCTAATTTTCATTTTCACACTGAACTGCTAGACTAC
ATTTGACATACACTGGTGACATTCAAAGGTATAGTTCTGGTAAAATAAAATTGAACATATGGTGGCACCAGCACTGAGAGCTTGGTT
CTTTTCCTGATCAGCAGTTTGGCTCTTCATCAGTTAACTGCCTGGGCCTCAGTTTCTCAGCTGTTAAATTGAAGGAGGTGGATGAGT
TATAACGTTCTTTCTAGTTCTTACACAGAATGAGTTTCTTGAGTTCCAATATGCTGGAGAAGAAAAATAGAAGAGTTTGGCCACTAA
TTTATAACAGAAGTAGTATATACCAGGACACGTGATAAATTATAGACATTTTCTGTTAGGGAGACTTGTCTGAAGACTAGTTTTATT
ACTTTCATTTCTTCCTCAAAGATCCTTTCATAAAAAACAAACAAACAAAAAACAAAAAACAAAAACAAAAAAAAACCAAGGCTGGGC
GCGGTGGCTCACGCCTGCGATCCCAGCACTTTGGGAGGCTGAGGTGGACGGATCACAAGTTCAGGAAATCAAGACCATCCTGGCCAA
CATGGTGAAACTCTGTCCCTACTGAAATACAAAAAACTAGCCAGGTGTGTTGGCGGGTGCCTGTAGTCCCAGCTACTCGGGAGGCTG
AGGCAGGGGAATCACTTGAACCCGGGAGGTGGAGATTGCAGTGAGGCAAGATCACACCACTGTACTCCAGCCTGGCAACAGAGCAAG
ACTCCGTCTCAAAAAAAACAAAACAAAGCAAAACAAAAAACCTTTCACTAATTTCTTCTTGGCTTGTTCAGTTCCTGGTATAGTGTC
CGTGCTTCTCTCTCCCCACTGTGAATCTCAAATGTAGTCCTGTGAGTCATTAAATATATTTTTATTATTAGAATTACAGCTTCATGT
ATAAAATCTGAATCCTTTTATATAACAGTTGTAATGACATTCTGAGTAACAAAGTCAACATCTTGTACAGATTCCTTTCTCATTAGT
TATCACAGGCTGGTGGGGGAAAACTTGTTTCCTCTGTTATATATTGGAACAATTCAAGACAGAAGGGAATTGATTTTATTGAGGGTG
TCACCGTACTGGGACATTTCTCAGAAAACCCTGAGACTGCTTTGGGTTTGGGGGAGGGAGAAGAAAGAGGATTTTGTTTTAAAGTCA
TTTCTTTGGGGTACCGAACACCACATCCCATCACTCATCATTAATAGATGTTTTGCCCTTTTGGATTCTGGCTTTTAACTGTTTTGG
ATTCTGGATACTTAACTGTTCCCAGGCAAGTAAATTGATCTCAATATTCTGGTTATTTATTGCTACATAACCTCCAAGCTTAGTTGT
TTAAAACACAGTTGTTTATTATTATAGTTCACAGTTCTGTGATTTGGCCAGGCTCATCTGGACAGTTCTCACTTGGGGTCTCATACA
GTTGTGGTGAGATGTAGTTGGAGTTGGACTTACCGGAAGGCCCTCTGGGCAGGATGCCTGAGATGGTATACTCATATGACTGGAAGC
TGATGCTTGCTGGGAGCTTAGCCAGAGTTGTCACTGGAGCACCTACCTACACGTGGCTTCTTCCTGTCTCTAAGTCTTAAAGAAGCT
TCCAGCCAGTTAAGGTTCCACTTAGAACTGGCAGTGCCACTTTTATCTATTCCATTGGTTAGAGCACTCATGCAGACTGCTGAATTT
CAAAGGGTGAAGAATTAGAAGCCACCTTTTGATGGAGAAGTGTCAAGGTCACATTGCAGAAAGTCATGTAGGTTGTGGCCATCTTTG
AAAAATATAGTCTACTGTATTAAGGCACTGAGCCTTAATTTCTTGTTTATAAAGGAAAGATAACATTAATGGTGTTGTAGTTTTAAA
GATTCAGTGAGATAATGTTTTATTCGGCATAATATCTCAATATTTAACAAATAGTGAATATCATTACTGTTGTTGTCTCCAGATTTT
ATACTGGTTCCATACAGGGCTATAGAACTTCTAGAATGCCTTAGTCTAGTACTTTGGCATCATTACTACTAGGTAATGATAATACCC
TTAAGATCTGCCAAGAAGATGAGGCAAATGGATTCAATAGATTTTATAGTGACTTGTTAGGAAAGAACTCAATGGAAACGGTGGAAT
GATTTGGGAGAACCAAGTCATTTTTCTGTTTTCTTTGCTACAAAGGATTAGAAAAGCCTCAGTTACTTGAGGCCAACTTTCTCTTTT
GATATGCTTTCAGATATTACCTTGTGTCTTTAAGTGCAAGTTTATCCAGGGTTGATGGCAGGATCTTGCCTCTAATTTGCTTCCACA
GGGCTTAATTCAGAGAGGAGGGAGACTCAGGAAACCTTGGGGCGGTGGGGGAGGGGGGGCATCAAGTAGGAAGCTTTATCTTATGAT
TCACTATAAATTGCAAGATGTTCTGGAATGATGATACATCAGCAGTTTATAAGTCATAATTTTTGTTTTAATTTGCTAGACATCTGT
TAGCTTCGATGGCAACCATATGGGAATCTAAATTGCGTTTTGAATTACAGGGAGATGGTAGGAAGAAAGCTAAATATGGTTATGAAA
GTGTCATATGGGAAAAGTGTAAGAAAGGTATAAAGTGGGGAAAGTAATTGAATATAGGCTTGCTTAGTGTGGCCACTCCCTCTGTGG
GGCAGTATGAAGGCGCAGAAGCATTCTCACTTGATATACTCCGCAGATTGAAATGAAGCCAAGTGGGGAGAAGTTGGAAAACCAAAT
CATGTTTCAACCACCATAAACTTGCCAAAATGTTGCACTCATTTACATGGGTCACTTTGTTTTAATGTCCACAGTAATAGGTAGTCC
CTGGCAAAAGGTGAAAGCAATCTGCATTTTTAACAGAACTTTTCACTAATGATGTTTTTCTTGTAAGCACCCCAAAGAGTCTTCCAA
GAATATTATATCTTTGTGACAGATGAAGAAATTGGAGTACAGAGATGTGGAGTAACTTTTGAGGTGTTGAAGAGCATGTCAAGGTTC
AGTTTCAGAGTGTTAAGTCTCTTCCGTAATGATAGCCCCAGCTTTTTGGGTGGAGACTTATTTTAGAAGATGCTTGTTATTCTAAAA
TAAAACATCAAGTAAGTCATTTACATCTGTTCATAATCGAAAAGTGATAAAGTTTTTGCATATGCCACAATTAACACCACCCACAAG
CACAAAAAAGTTAGGATGAAAATGTTCTTCAAGAATATGGTGGAATAATTGTAGCCTATTGAAAATTCATAAATGAAAATTGGAGTA
TTGTAGACAGCATTCTTGAATCACATGTTTGTTTCTGTGTGTAATACTGATGACTGTCTTAAAATTTCTAACTTCCAAATAAATCAT
ACTCCACTGGATTATAAGGGCTTCAAGTGTGGGGGCTGTCTCACTTTCATAAGCCCAGTGTCTTGCGCATTGTCTGAAACACAGTAG
ATGTTTTGATAAACGTTTGGGCCAGGCTTGGGGGCTTACACCTGTAATCCCAACACTTTAGGAGGCTGAGGCTGGAGGATGGCTTGT
GCCCAAGAGTTTGAGACCAGCCTGGGTAACATAGTGAGACCCTATCTCTACAAAAAAAAATTAAAAATTAAAAATTAGCTGGGTGTG
GTGATGCGTGCCTGTAGTCCCAGCCACTTGGGAGGCTGAGGTGGGAGGATCACTTGAGCCTGGGAGGTTGAGGCTGCAACTGCACTC
CAGGGTGACAGAACAAGACCCTGTCTCAAAAAAAAAAAAAAAAAAAAAAAAAAGTATGTTGAAGGAAAGAATTACAAAATCATCAGA
AAGTACGTAGGAATCATCTCCTCTAACTTCCTAGTCAGTGAAGCATTCTCCATTGTATCGTGCCTACCTTTCTAAGCATGGATCTGT
TTTTGGACCACTTTAATTGTAGAGTTTCATCTGTTCAAACTAGATGTTATCTGTCTCCCCATCATTTCCATCCTCCTAATTTACTGT
TCATTTAAAATTTTTATTTGGAAACATTAAACCAATAGGAAAATTGCAAGTAGTATAGAGAACCTGTTTTTATCCTGTTTGAGAAAA
AGTTGCCAGCATGTTGGCTATCACACAAACAGTTGAGTATGTATTTTCTGTAAACTGGACTATTCTCCTAGATCACCACAGTACAAC
CATGAAAGCCAGGAAATTACCAATGATACATTAACACCATTTAATCCTTAGATTTCATCCAAGTTTCCCAGTTGCTCCCATAATGTT
TTAAAGCAAAAGAATTCAGGTCAGAACCACACTTTGTATTTAGTTACATGTTTTTAGTCTCTTTCAGTCCTTCACTATCATGCTCTT
GATACTTTTGACAATTATAAGCCAGTTATTTTGCAGAATGTCCCTCAGTTTGTGTTTGTGCCATTTTCACAAGCTTAGATTCAGGCT
GTGCATCTTTGGTAGGAATAGTACAGAAATGGTGCTGCGTTCTCATTGTACCCTATCAGGGTGGCATTGATTTCTGTTTGTCCCATT
ACTGATGATGGTCACTTGATCAAGGTGTTGTCTGCCAGGCTTCTCTACCATATAGTTACTCTTTTCTCATTTGTAATTTATAAATGT
TTTATGGCAAATTACATTGAAACCATGTAAATAGCCTATTCCTCATCAAATTTTCAATCTGTTATTTATTTATATCAGTAAGAACTT
AGTGTCTTCCTTTTTTCAGTGTGGTTTAATGTTACTGTCATTACTTTATTTAAAATGGCCCTAGGTTTGGCCAGTGCAGTCTTTTCA
CATTGGCTCCTGTGCTCTTTTAACTCCCATCAGTCTTTGAGCACCATCTTGCTCTCTGGCAGAATAAGATGTTCCAAACTCATTTTG
TGATTTACCTGTGTCATCTTTTGTATCAGACATTTCTCCAAGGAGCTATTGAATGGAGAATGGAATTTAGAAGCCAAGATCTGGTTG
CTTGGTGTGCTCATTGCTATTAGGGTGTTGCTACTCTGTGTATATACAGACACACATGTACATCTGTAATTCTGTATCTATGTACTT
TTAAAACCATGAGTTTACAGTCATACCTTAAATTCCTGTCTGATACCACAGGGTTTAGTTTTTCCTCTCCATATTTTATAACTGCTT
TATTTGACAGTGAGATTCTTGGTTCGCATTATTCTTAACAGTTTACTTATTTGGTAAATCTCCCCATATGTAACCAATCTTCTGCAG
CTGCCACCACCTTTCCTCCTGGGGCTGCCCTCCTCAGCTGTTAGTGCTCTAGCATCCTGTGCCACCACCCCCATCCCCATATGGATA
CCTTTCTCCCTGAGATACCCTGCTCTGGGCTTCCACAGCATCCCTCTTCCTACGTTTTTGTAAACGTAGGAAGCCTACCAGATTGGA
CCTACCTAATGGCTTTGAATTTTCAGGAAGGAGAAGGCGAAGAGCCTCGGCTACTCTTGAATGATGTAAAATAAGACTTATCTTCCT
TCCCCATGGTCCTTCATTATTTAAAAATAGCCATTATGTCATTCCTAAACATTCTGTTTTCCACCTTTAAAAGCTCCTAGTTCCTCC
ATGTGTTTACACTAATGATGTTTTTCTTGTAAGCATCTCAAAGAGTCTTCCAAACATATTATATCTTTGTGACAGATGAAGAAATTG
GAGTACAGAGATGTGGAGTAACTTTTGAGATGTTGAAGAGCATGTCAGGGTTCGGTTTTAGAGTGTTAGGTCTACATATACTGTTTC
CAGATTGTTCTTTGCCCTGGTCACGGTGCTCTGCCTAGGGTCCCATTTGGACACACCTCTATTAATGCAGCAACCAGAATGAAACAC
GTTGTTCACAGGCTTTTCTAACCATCCGAAGAGCAGCAGGCTCTTGAACTCTCCCTTCAGTCCCTAGGCTGTGCTGTGCCTGGTGGC
AGAAGCCTCTGCTACTGTCTGGAATTGACTTTGAGTAAGTTACTTTCACACATTAGTTCCTCAGTTGCTCATCTAAAAAAACCAGGA
AAATAATTTTCTGCAAAGTTGTTATCCAGTATTATGTGAGATCATGCATATCAACGTGCTTCATAAATGGTAAATCTCTAAATACAA
ATACTAGTTATTAACCCAGGCTAAACTGCACTTGCTTTCCCTGGCAGCCAACGAACACTTTGGCTTGGATTGACTTTTTAGTGAAAT
AAAACCTTCACTTTTTCAAGTGAACTCTATTAAACCATCCCCCTAAGCTATATGCTAATCAGAAGCCCTAACCCTCCAGTTAATTAT
TTGAATTTAGATTTAGTTCTGTTTATTTCTCCCATTTAAATGTCTTATTCTGGCCCATTGGATTTCAAACCTGCTACCAGCAATCCT
AGGCCTCCTTGAGGCGTATGTGGGTGGGTGGGTCTGTGCTGGTGGGGTGTGGACTGAGCAGTACCTCTCTTTCCTCCAGAAAAGCTT
GGTTATTAGTTTGATATTTTGGGATACTAAGAAAGATTTTGCTTGAAAAAAGGGGTCCCACCACTTAAGAAAGAAAAGAACCAGTAT
TCTAGCCTTTAGATGTCTTTGAATCTTGATGGCCAAAGTACATTTTTAAAAAAATTACTCCTCGTGTGGGTTTTTATCTCTTGGAAT
CTCTCCATGCATGGGAAGTCCCTCTCACTAACTAGGGGTCACTTCTGTAGGATTAGTTGATATTTCACCCCTTTATGCATGTAGTGT
GGCAGCCTGATTAGTAAGGAAATTTCAGTCTTCCCACTGGGAGATCTGGTTCAGTAGGTCCATCCAGGATGGGGTCCTGGAATTTCT
GTTAAGAAAAAAATCAAAACTTCCTGCAGGTCATTGTGATGCCCAGCTGAGTTTGAGAAGCTTTAGCTTGTTTTTCTTGGGAAGATG
TGGCCTCTCCTTCCCGTTGCTCCCTGGAGGGGATGTGTGGGAGAGCTGGGCGTGGGTGGTGTAGAGCGGTGACCATTTCACCAGCAC
ATGAAGCTAGTGTTCTTTGCTTACGAGAAAAAAAAGGTGGCCTATTTTATCCCTCATGGAATTTTTTATTAATCCAAAAACGCCAGT
TTATTTTTAAACCTGAAAGCCCTAAAAACAAAATGAGAAAAATCTCTTGAAAGAGAACCTCTGGTGCTGCAACTAGTTTGCTAGCAG
TTAGCATCATATAGGGCTAGAAGCTAGCCATTTATTCGATAGGCACAGTGAAGAGCTAGACTTCTGGTTCCTTTGCCCTGCTTGTCC
TGGACCACTCTGCGTGTGTCAGTGCAGTTGAGTAGCTCTAACTACAGACACTTAACAAGGGCAGACCACCTTCTCCTTCAAAAAAAA
TCAGCCTCCCCATCCACTGTTGCTGTTCCTTTCAGCCTCCCTTTTCTGGGGCTTTCTCGTTGGCCTCCAGCCCTGACTATGCTCAGT
TTGTAGGAGGGGAGTTGCTTTCTGCGTCAAGGTCTGCATGACAGGTTCATTGAATTCCCTCTGCTGTAATGCTGTGGAGGGGACATT
TCTTTTCCCATATCCAAACTTGCAGCACTGTCACAAAGACAGTTGACAATAAAGCAAATACAGAAGACCCAGACATGATATTTGCCA
GTGTTCTTTAGAAAGCTTCAATGTGGAATATCCGGGTTATGCCTAGTGTTTTCCTGTTATTTTCTTAACTTTCTTTTTTCCTTCACC
CCCTTTGAAAGTTAGATCTTTGATTACGGCTGGGTTCCTAAAGTAGAAATCTTCAAAAATAACATTTTGAATAAAACTCTGTATTAT
TTCTCTGAGCTGTTTTACTGGGTCTAACACCACTCCTTTTTTTTTTTTTTAAAAACAAACAAAACAGAAAGCCAACCAAACAAAACC
AAACCAGAATGGTATGCAAACCCAATGGTTAGCACTGTGATATTATCCATATGTACATAGAGGATTGTTAGTTATGAATCACAAGGT
ATAATTTTTAAGTAATATAATCACTAAGCCCAATTACAAAATGGTGTTTTTGTGGATGCCAAAACATCTACAAATGGCAAAACATTT
TGGCTCTCTTCATTGGAAGGGAAAAGTGATGTTCTATACCACTTCCAGCTACTATTTACTATTTTGATAAGTATTAGAAGGCATAGG
GCTCTTTTTACTTGTTGGAAAATTTTTCTAAATTACACTTGGCACTAAGGCTTGAATTTCTAAAAATCTGGAGGGAACAGCTGGTGC
TCTAATAAATATTCAGGCCAGCTATTTTAATGCATTGTACTCAATTAAGTAGTCCTAATAAAAGAGGAATACTTGACTTAAGCATTT
CTCAAAGCATAATGTTGTGCTTCCAACAAAAATTTCCTCCAACTGTTGATATTTCCCCCCAAGCAGCCTTAACATTTAATTCTGCCA
AGTAAGTAATCTAATCATGGATGATGCATTTAATGCCAAAAGTTAGTTCTCTTTTAAGGTATAGTTGTATCAATGTGGCCTAGCCCT
TTAACTGGAGACTCCATAGGTCACTGACCTTTTAGAAGAATTCTTCGTCTGGTTCAGAGTGAGAGAGTATTGAAAGATTCATCCTAA
CTTGGGCATTCTGGTACCCTTGTGGTGGGAGGGCAGAAAGTCTGCTGTTAAGGAGTTTAGACTTTGAGTGTGTCTGGATTTTTACCC
AGCTATGTTCCCAAGCTTGTGACCTTAGACGTTACTTGCCCACCTTGCAGTTAAGTCTCCCCTTCATCTGCAAAATGGCACGTTTAG
TAGTATTAATATTATTAATAGTAGTATTAAGATAACGTCTATCTCGGAATCCTGAGGATTTGAGGAGTTAATATGTGTAAACTGCTT
GGAATAGTCAATAGGTATTAGCAATTATTATACTGTTTTCCTCATAATTTATTTTGATTGTTTTCCCTTCTTTCAGAATCATTAAAA
TAATCTTACATTGTAACATATATTTTTTGTTCTCTCTAATATTCTGCCTACATAAGTGCATTCTCAAAATAACTTAAAAGGAAAAAA
AGATCTAACAAATGTTCACAGTGTTCTTTTCATTGTAATGAGTGGCACGCAGTTCTTATCGTGGATACCAGACCATGAATTCGCTTC
TATTGTTTGTTGTTAATTTTCCCTTTTTTCTTTACCCAGTTACATTCACTATTTTCTTTGCAAAATCAAAGTGTCTCCTATTAGTAT
TTTGTACTGGGAAGTTGAGTTTCTGATGGATAGCTGTGGTGGTGTTAGGCCTAACACCTGGGTGGGGTGGGGAGGATAGATACCCTT
TGTCAACATCTGTTAAATCTGGGCATTCTGGAACTATCTAGATGGCTGTAACTAAGTGTCTTTAGTTCTTTATGGTATCATAAATCC
TGCATATTTTGGTCATTTTTTTCCTCTATTTCAGACAAATTCTTGAGGTCATGAAGGTGAAACTTGAGTCAGTGATCCTGATAACTT
GGGTTTTCCCCTTATATGTCCTTCCAGCTGATGCAGAGCTAGGATTTTGGATTGAACTACCTGGATCATTGACTAATCATCTTGGTG
AATGAGAGACTCACTGGCTTCTCCTCTAGGGCATAATGGGATGCGATGAGAGCTTGTGCTTTTTTTTTTGCCACCTGTACTGTCCCC
TGACACAGGAGACATGGGCTGTCACATTTCCAAGTTGCCTCTTCCATATAACTGGGCTCAGCTCTGAGAGGAAGGCCAGGGCGTGCT
AAATTAGTAGGTGCCTGTCACTCATTCCCATGCAGAAGGCATGCTGAAAACCCCAGCCCTTCAAGGGGAAGCAGGGGATAAAGGGAT
AAGTCTAAGTAGAATTTTTACTGTAGTTTTTATTAGTAAAAAAGAATGATATATGTAGCACAACAGGAACAGAAGCGTATTTAAAGT
CATCTCTAGCTTACCAGAAACACCCCTAAAACTGCTGGTTTAGATTCATTAATCAACCTCATGGTCCTCAGATGGGTTGTGACTCCC
TGTTGCGTAAACACGAACCATGTTACTTCCTGGTCCTCCTGACCTCCACTAGCTGATTTCAGCGAGGATCAGGAGTTAGGTCGCCAG
CTGATCTAAAGCAGCGAGTCCCAAAGTTCAGCCATTTGTAAACCACCTGCATGGTTTTGGACCAACTCTTGTGTCACCCCATAGTTT
CTCAGATTTTAATATGCATACAAGTGGCATGGGATATGTTGTTCAAATGCGGATTCTGGTTCAGTAGGAGTGAGAGGGGACAAAGAT
CCAGTACCTTTAACAAATGCCCAGGTGATGCTGATGGCTCTGGCCCACTGATCTCACTTTGAATCTCAAAGATATAGTCCATCTGAA
ACAATCTTTTCTTTTAAATTGACTCTGATTTTAATCTTAAATACATATATTTAAAAAGGAATGTAAAAAGTATCACTATTCAAATGC
ATATAATGACAATAATATGACTTACTAACTTCTAGGTAGCTATTATTGGTGCCTGTTGGTAAGCCTGAGTCAGAGGCCTCTTTTCTT
TTGTTATAATGGAGGTTAACAAGTGTAGCAAGTGTTCGACATTCTGGCATCAAATGGAGACTTTCTCCTTAACTAGACCAGAGGATG
AAAAGATAGTTGAAAACCAGTGACTTTATATTGAAGGATATTTAATGCCACGTTCTAAAACTTCTCTTCTGGTACCCCAAGTCACCA
GGGAATCGCACTTTGGGGAACACTTGTGCTTAAGTAATTTGCAGCAAGGCATGCATTTATAAACTGAAATCTATTAGTGAGGTGGAT
GGATCCCTAGAGGTAAAATGCTTGGGGAATGTGTTTAAATGTTTGCATACTCCATGTTCTGTTTTCTGTTGAGAGTGTGGGAGAGAA
GAGGGGCATCCCCCATTTCTGAAAAGTTGCTAACCCTAGGCCCAGTGAGTAGTTACATCTTAGGTATGAGAAGGAAAATGCTTTGGG
AAATTGTATCTCAGGCCAGCAGGAAGTCAACTTTAAATGCTTAGGAACTGTATCTCAGTGGGCTTCCTCCTCTCCTCTTTCAAGTTA
CTATAGGTATTGCTTATGGTCAGCTTTGGAATGAGTCTATTTACATAATGCATTTTAAAAATAGGGTGCTTTTCATTGTTCAATGAC
GAGTTAGTGGGTGCAGCGCACCAGCATGTCACATGTATACATATGTAACCTGCACATTGTGCACATGTACCCTAAAACTTAAAGTAT
AATAATTTAAAAAAAAAAGGGTGCTTTTTTATTCCAAAAATACGGGAACATCAATATTGCTTTTTTAAAGGAAATTTACTACAATAA
TATTTTCTTTCGGTTCAACTGTATTAAAAGTGGAAACTCTTCGTCAGGTGCTCCGAGAACAGTGAACAAACACTTCAAATGAAAAAA
ATATTTTAGAGTTTCAGTTGTATTTCCCTATGAGGCCTTGAATTTAGCAAACCTTAGTTAATTAACTTTTTAAAGGAAAAATGTGCT
CTTACATTAGTGGCTTTTAAGCATTTCTTCCGACTTTACCGGCCTTTCCCCCTTAGGACAACATTTAAAATAAGTGTTTTCAACAAA
CAGAGCAAATACCTTGTGAATAAACATCAATTTCACACAACCAGAAAAAAAAAATAAATCAATAAATCTCCTTTAGGCATTTGAAAA
ACTCTATACTTTTTAAAAAGGGAGGAGTTGTAATTCAGTAACAGGAAAACTAAACCCCAAGAAACCAGGCTGTTAGTTTTAGATGTT
TTTCAGCATGACCTTATGGAGGCAGAAGACGGGCTGGGAACTTCCTGAGCACCTGTTTCGTTATTGGTGCCCTACTTATCCAAGGAT
GGGCAACAACTGCCCACAGGGCAACTCCTGTGAGAAAGCCTACTCTGTTTTTGTTTTTAATCTCATAACTGGAATCCTAGGGAAAAT
AGTGTTTTCTTTGGTACCAAGTTCTTTTGGCTTCATCTATTGGGTTTTGAAAGAATAAACTGAATGAAGAGCTGTCTAGATGATACA
ACTATATGTGGCATTTTCATATTTTTTCCTGACCCTGCAAGATTAATAATCCCTAATATTCTCACTATATGCTTTGAGAGTTTTGCT
GTCCAGTGAAGATTTGAAACAATTGCACAAAAATTTGAAAACGGTGTTTTACTTGTCATGTTTAAAGATACAGAGTCACTATTTTAG
AAGTGAAGGGATAGGGGTCTCAAAACAAATTGAAATATATTTTGTGAAATATATACATATGTATTCCACTTAAATATAAAATATATA
GTACCAATATATATCCTTGATGCTTATTAAATACTGATCTATATCCTTGGACTTTTTAAAAAAAGTACAGTCATGTATCGCTTAACA
ACAGGGGTATGTTCTGAGAAATGAGTCATGGGATTTCATCATTGTGCAAACATTGTAGAGTGTACAACACAAACATAGACGGTGTGT
AGCCTACTACACACCTAGGCTGTATGGCATAACCTGTTGCTCCTGGACCACAAACTTGTATAGCATGTTATGGTAATGAATACTGTA
GGCATTTATAACACGATGGTATTTGTGTATCTAAACATACACAAGGTACAGTAAAAATATAAAAGATAAAAAAGTGGTACACCAATG
TAGGGCACTTACCACGAATGGAGCTTGCAGGACTGGAAGTTGCTCTGGGTGAGTCAGTGAGTGAGTGGTGAGTGAAAGGGAAGGCCT
AGGACATTACTGTACACAAATGCAGACTACATTAACACTATACTTAGGCTACATTAAATTTACAAAAAATATCTGTCTTCAATAATT
AACCTCAGCTTACTGTCACATTTTATTTTATTAACCTTTTTTTAGCTTTTTTACTCTTTTGTAATAACACTTAGCTTAACAGATTGT
ACAGCTGTACAGAAATATTTTCTTTATATCCTTAGTCTATAAGCCTTTTTTTAAAAATTATTTTTTATCTTTAAAACTTTTTTGTTA
AAAATGAAGACAAAAACACACACATTAGCCTAGACCTATACAGGATCAAGATCATCAATACATCACTAGACAATAGGAATTTTTCAG
TTTCATTAGAATCTTATGGGACCACAGTTATTCCGTCATTGATTAAAATGTCGTTATGTAGTGTGGGACTGTAGATATGTGCTCTCT
TTGGAGAAAAAAAAAGAGAAACTACCTAAATGACCGTATCACCTCTAAATATTTTCATTCATAAGAGACTAAAATTTCTTTACTGTA
GTATACTATAAATATGCAAACTAAATGAAACTTGTTTTAAAGTTAAAAGCTGGAGATCCATTTTAGAAACTAACACATAAGCAGAAA
AAAAATTTTGTGGTTATTAGTTGGAGGCTAGAAAAGAAACTCTTTGAACTATGAAGTTACTAAGTCTTAGCTAGAGACCCCAACAAA
TGATTAATTATTGCTAAGAATCTTTTAAAATCCAGTTTTAAAGTTTAAGTGTTGTTTCTGTGGTTTTTTTCCCTCACCTGCCCCCTT
CTTTCTGTGATTCTAATTCCAAAATTGAGAAGACAGAGCTTTTTACTTTTAGGGAAGCTAAACCTCTTTCTTGCTCTAAGAAGGTTG
ACCTGCTGATGAAAGAACAGAAGGTTGGAATTCTTTTGAGCAGGAATAGCTGTGCAAATGCTTATTTCATTGTTCTTTGGCCAATCT
TTTTAGCACTCAAGTATTAGAACTATGCTTTAATTGGTCTTTTGCAGCCCTGATTGTATAATCTTAGGATTTATATGTTGAAAGAGA
CCTTACAAATAATTTGGGTCCTAGTTTTTACTACTCACTGTTCAACACTTTGCCTACCCTTTTCATAGTGGAATCCTTTGTTTTCCT
AACCAAAATTTTAAGCAAAGACCCCAGTTTATAAAACAAAGAACAGAGCTTCTCTTCTGGGAGCCCAGCATCTCAGGTGCCTCAGCT
GTGTTCTTGGTATCACTGTGTAGGAACAGTGTGCAGAGGGGACAGTGGGAGTCCTGGTGGATTTTACCCATGGGTGGAGTTTGAGCT
CAGACTGGTGGAGAAGGCATCTGCTTACCGTTTATATGTTGAATTAGGATGAAGTCGGCACTTGGCCTGTGTTCTTTGTAGGATGTG
TGCACGCTGAATTGTCCAAGTTCCCAAGTCCCAGCACTTTGACTGTATTTGGTTCCACTAAATGGTTTTGGCGCAGTCGTTTTGATT
AACATCAACCTTGCTTATGTTAAGTAACTATTCAGGTTATTTATTATGCATTACTCTCATGCTTTGGAATGAGCAGACTCAAAAACT
TCTGCTTCATATGGATTCCATATGTGGCCTTTAAGTTAGCAGAAACACAAAGGTTTCCAATTAATTTGCACATAAAATTGTATATTG
AGTAAGTTAAGCTTGATGGCATAGGCATTATTTGAAATTCTTTGTCATCTTTAATATTTTTATATTTCATTTAAACAAGTTATTAGG
TGTTTGAAGGTTCTGTGGTGACGAACATAACCTACACATTCACATATGTCCTGTTTGGTTTTTTTGTTACAGAATGCAGGTATCCAT
GGAATTTATTTTATTGTTTTATACTGATGAGCTCCAGATAGTAGGAGATTATATAAAAAGAGGGGATATTTACTTTTTTATTTCTGT
GATTTAACTTTTTCTCCCCTCCTCTTCCCAATGCTAGGCAGAGAATATTACAACATGTAAGACAGTGGTTAAGGGTGAGACTGCCTG
GATTCAAACATGGGCCTTGATATGTCCAAGCTGTGTGACCATGGATGGTTACTTCATCACTTTGAACTACTCCTTTTGGGCAAAAAG
TGGGTAATATTAGTGGCGACCTCTAGGATCATTGTGAAGATTCCATGAGATGATGTACATCCAACCCTGAGCACTCAGTGAATGCTG
ACGTGATAATTGAGGGACCAGTAGGTGACCCACTCAGGGGAGTTGCGGGCTTTTGGTTCACTGTCACCTGGGAAAAGGTGTTGTCAG
TGTCCGGACCCTCAACTTTCCTTGTTAGAAAAGTGTACCCTGTATAATAGAACTTGTCACTAATGCCAGGCTTTGTTTCCTTTGTCC
CCCACCCATTGGAGGAGAGTGTATCTCCATGCTTTCCTTTGGCAACTGGATCTGAGGGTTTTCTCATCAGAGCAGCTGCCATGTTCC
TCTCCTCCTCGGCTGTAATTTCCTCTCCTGTGTAGCCATAGCTGTCCACCACGTCCATTCCTCACATGGCCTGGCCAGTCCTTCCTT
TCTGGATAACAACGCCAACATTTCTTATGTCTTACCTCAGGGATCATTTTCAGGCCACAGACCTACCTGTTTTGTTTTGTTTTTTTA
AAATGGTAGTATCTACAGAACATTACAGTTTCCGTAAAATCATGTATATAAATATGCCTAGAAAACTACCTGAAATATAGCAGCTAT
CCCATAATTACTAATTGAAATACGTTAGTTGAATCTGAAATAAATTTCTTAGCCTAGGAACAAAGTTAGCATCCATCTGGGTCTCTG
CAAAAATTTGTAATGTGTAAAAATAGGCATGTTTAGGAAGTTGCTTAATTTATTAATATATACTTCATTTTTATGGCAGTTACAGAT
TCTCTAATTGAATTTATATTTGCATGATTACAGTTTTTTTTTTGTTGCAAACGATTTTTATGGGCTGCTGAAGTTTAAGGACTCTGG
CATTTTAAATTGTTCTTCACTGTCTGAGAAGTAAAAATATGTCTTAAAAAATCATTGATGTTATAGTAAAGGAAAGCTCCCTCCCTT
CCCTAAGTTAGTGCCTTTCAGAATGTGAGCATTTGTCAAGTGGAATTCTGTCTGATCCTTTTTCATTTTCTGAGCTATTATAAGTGC
TTTCTTTTCCTTTAACTGTCATAAATGGTTTTTCTTCCCTTAACTAAGTAAAGATTTCTGGAACAATGTTTTGACCAGATTGAAACC
AGCTTCTGTCCTATCTACTAGTCATTCATTTCTTGATTTCTCGTGCATTTCTAAACATAGATAAGTTTCTAATGGTGAAGAAAGTGA
AACAGAAAGGAAGGAAAGAAGCAAAAGCTGGGTTGGGTCACCATATTGAGCGAAGGATATGCTTGTGGGGAATCCTACACTGTGATA
TTGGAGTAAACAGTTACATTATGGGGTGAGACCAGGTGAGATGGCTTATGCCTGTGATCCCAGCACTTTGAGAAGCTGAGGTGGGAG
GATTGCTTTAGGCCAGGAGTTCAAGACCAGGCTGGGCAACAAAAATGAGACCCTCTCTGTACCCCCCCCCCCCCCCCCCTTAGCCAG
TCACAGTGGGGTGTGCCTGTTTTCTCAGCTACTCTGGATGCTGAGGCAGGAGGATTACTTGAGCCCAGAAGTTCAAGGCTGCAGTAA
GTTATGATTTTGCCACTGCACTCTGGCCTGGGCGACAGAGCAAAACCTTGTCTCAGATTAGATTAGATTAGATTAGAATAGATAGAT
AACATAGATAAGATCAGTGTTTCAAAATCCTTTTCCAGTGAGAGGCATGTACTGCTGCTTTTGTAACCCATGACATTGAGAGTGTAG
CAGGGGGTTCATGTGGAGGAGTTGCTGAGACCAAGAACCCCGTAAGGATAGTCATCTAAGTTTGGGAATACACACAAATTGTTGGGA
GACACTTCTCCATGGGTCTCTTGTGCTCCTGCATGTCTTACTGAGTATACCAAGAACGTTCTTTACCTGGGCCATTTCTTCGTGTTG
TGTTTGCAATGAGCAGCCTTGAGGGATGAAGTAGTGTCTCTTTCTGGGACAAAGACTGACCTACTCTCACTTACTGCTTACTATAAA
GCAGCAGGTTTCCCAAGCATAGGTTCCTCAGCTGGATGCACGCTCACTGCATGTGCAGCTGCCATCTGGGCCCCTCAACGCTATTTC
CATTGGACTGATGTAAACATGATGCTGGTGTTGTCTGAATCATGCTGTGAATAATAAAGTCCTTTGTCCTTGACCCGGGAATCTCAT
GTCTTCTGCCAGCTTCTATGAAACAGTAACAGGCTAACTTATTACCTTGCGAGTAGGGTAAAATCAAACAAGACCAGATACAGGTGG
AGCTTCCAGATGAGCTGAACATGTGGAGGTTCCTGTAGGGTGGTGCTCTAGAAAGGGCATGGAAGCGCCATGTCCCTTGCCCCATAC
CTTTACCTGTGAATCTCTTCATCTGTATCCATTGTAATTTTTTAAAATAATAAACTAGTAAATGTAAAATAACCAAAACAAAGAAAC
CAAATGCCGGTGTATTACATATAAGTTGATACCTAGCCAGTTAAATGCGTTCAGATGTGTCTTGTTTAGGAGGTAATCACCCGGGCC
TCCCCCACAAAGTATGGTTCAAAGGGTCATGGTGGGTGGCTCCTCAGAGCATCTCAGCTCCCTTAGACCTGATGAAGGCTTCACAGG
CATTTCATATCTGTGCCTCTGGCCAGTATATCTCCAGGGAACTGAGTGCCAAGGGAGACCAAGGCATCTTTACTGACATTGTTGTAC
CTTAAGTGAGGCAACATGTAAAACTGTGACACATGGGAGTGCTTGATTTTTGAGCGTGCGGAGCAACAGGGTCCAGTGTTATGGTGG
CAGGGGCAGGAGAACTCATGGCATCAGGAGCGTAGACAGCCCCACTGGGCACGAATTATAACCCAGGCAGAGGCAAGGCGCAAGGGT
GGTCAGACATCTGGCCCTGAAATAAGAAGAGACAGGTGGGAAAGTCCAGGCCAAGACCGTGGAGCAGAGTTAAGAAGGAGCAGGAAG
AGCCGATCTCAGAAACAGGCTGGCTCTGTTGAAATTGGACTGAGGCAGATAATTTCTTTCTTTCTTTTTCTTTTCACCTTTCAGCAC
TGTTGGACATGAAGGAAAGATAATTTCTAAGTTTACTTTTTGATATTTATAGATAAAATGCTTTCAAGCAGTTAACCAGCCTATTGG
AGAGTGACACTTAGATCATCTTCAAGTTGGTTTGGGCGATTTAGAGAAACATTTATAATTAAGTTATGCAGTTAATGAAAGCCAAGT
GGGGACATTTGTTCAGAACAATTGTTTTTGTACCTTTTCTTTGTGATAGAGCTGTAAAAGTGTATTTACTTGTAAACTGCAACAAGT
TTTTGCTCCTTCCCCCATCAAAAGAATAATATATCTATATAATAATTAGGCCTGAAATTAAGATTCTAGATTTAAAGAGGACTCCCA
GGGCTCAAATATAGGAGATAAGATGTTGAAACACCACTCCTGTTGTTTGCAAAATGAATATTAATGACATTTAGTCAAAACCAAATT
CTAAATATTTAATAATATGTGAAAATTATTAACTGCCTGTGGTCATCAGTTGAGACCCATAGTTACTGCTGGAATCCAGGGATACAG
GGATTAAAAAAATAATGGATCAGAAAGTAGATGTTAATGGGGAAGTATCTGTGGGAGAGTGACCTCTTATCCCTGAGGGCCGCCATG
TACAGCTACACAGGAGGCGAACTGCACAATTCCCTGGAATGTGCGTGATGCTCCCTGGAGTTGTGCAGTGTGGCAGTGCTGTGTGCA
TCAAGGGCAGGAGCAACGGCAGTCCTTCAAAGGCTTAAGTAAGTGGCATAGAAAGTGGACAGTCCTTTTAAGTTGCATGGCTTCAGA
CACTTCTGTTAGTGTGATAACTACTGCCTGTGCCTCTCACTCTTTGAAGAGGAACTTGCTGGTGATGGGGCTTCTCATGGGGAAGCT
GGGAAGCTCCCTACCAAGGGGGAGGGTTCCTAAGCCTGACAAGGACTGCTCATCAGGTGCTTTGGTATCCTGTAGACTGGGCTAAGA
AACCTCACACCAGGGTCCACTCTCCAGCTGGTCTCCACTGGGCCTAGGGAGACCAAAAGACCTGCAGTTGCTGAACCTCTCTTATTG
TACCTGTGAAGCCAAGTAAACCATTTGTTCACTTATGTTAAAGAATAAGCTTGATCATTCATGTTCAAAATAGTTAACTCTTTTTTG
TGAATTAATAGTCTGGCTATAATTACATTTGACATTTCAGTCCAGTATATTTGTCATCCAGATGAAGAAGCTGCCAGCCGCATTTTT
TTCCAGCAGTTTTACTTGACTTTCTTTTATTTTATGTACAAGGTGTTATTTAAGTGCACAGATTAATAGAGATAATTTTTATAATGT
AAAATTATTTTTATTGCATACCTCTATGAGTAATTTCTCACTTGCATTGTTATAATACCTCCCATCCTTCCCTAAAGCGCCACTCTC
CCCACTCTACTGCAGGAGATTTCTCTAGCAGCAATTCTGATTATGTCATACTCCCCTGCTTTAAATAGCTAAATATTCTTAATTTGG
AAAAGATTAAAGCTAGGCACCATTATAGGAACCTTGCCTACTTGTCACCTCTCATATTTTCTCATCCATATTCTGTTCCACAGTCTT
ACCTGCATGGAGCAAAGGTGTAGGGCATATTTGATGTCCATTATAAAATATTCAGGCTCCCTCAGTTCAAGGGTCCTCCACTGTAAA
GCAGTCCACTATGTATGCAGGTGTCAGTCCTTTTTCCACCACCTTGTGGGAATTGGGACTTGCAGAACTGCCACATTATGCCCTTAT
TTACATTTCTTGACTCTGTAGCTTTGCGCTTACCTCTGCTTTAAATGATATTTCTGCCATTTCTGTCTACCTTCCATCAAAGGTATG
GTGTCTACCTTTACAATAAGATTGTAAAGAATAAAGAGGTAATGAGTACAAAGCACTTAGCTCAGGGCTTGATTAGCTGCTGCTGCT
ATTATTGTTACTCTTGCAAAATTGGTTCCAGAATTATACCCTTTGGGTGCTCTTTCCTAAACTTCTTCTATCCCTACCCTATTATTT
AGGGAGACCCTATTTAGCAGTTCAGTCTTTATATATTCCTTATAGCTCTTTGTTGGCAATATTTTTAAATCATAAAGAATACCTAAA
TCTATAATGATACTAAAAAGAGTATTTGTCTAATTTTCTTCTGGCCAGGTGGATGGCATCTATGAAGAGGGGGCTGTGGAACGCTCT
CAGCCAAAGAGTGCTAGTCTGGCATGTTGTGCTTTTTCAGTTTGAATGGCTCTGGGCAATCTGTTTTGCTGTAGTCTCTTTTTTTCC
CCCGTTCAGCACCTTATTTTCCACCTGATGGTCTTTGATTCACAGCAAATTGTTTGTCTTCTAAAGGATAAATCGAAGGTGGGTTTA
AATGGTTCAAATACAAGCTGGCTATTAAACTGTGAGTTTTACAAGCTGTCATGTCATATGGCCTGAGTTCTATCTAGCTAGATCCAC
CATTGGAACTATTCTAAGCCATGGTGAAAAGATAAAAGACATCAGCCAATAAATCCCTTCCTCATGTAGGGGATTCATTGTTGGGAT
AGTACCTCCACAGGGTGTACTATCAGGCCCAGGCTTGTCACCATGACCTCTTTGGTGCTAAGTCAGTTAGAGGACCCATTTCTCACT
GGTCTTTGAGTTCCCCATGGGATAGGACCTTAAGGGGGTTTTATCCCAAGCTTTGCCAGGGCCTGGCTTCTGGAAGGCATTCCATAA
ATTGTTTTGAATGCATGAATGACTAAATAAGAAAATGTGAGAGTGCTTCGTTAACTGTAAAATTAGCTAAATGTCAACTAATATTTT
TTCCCTTCTGTGATGTGGAAAGATATGCAGGTGCAGGAGCAGGCAGCTGATGGTGGAGACTGGGGCGAATGGAAAGTGCCTGTTATA
AATGGCAGCCTCCTTACCTGTGCTTCTGTCCAGCTCTCTTGCCTCTCACCCTGAAAGGCTGTGGTCTGTCAAATCACTCACAGGTCT
GTCTGTGGAGAAGTCCTAAGTAGTATAGGGTCCCCACTTCTGTGATCCATTTCAGTTCCAACCCAAAGGACTTTCCAAAGAATGGAC
ATCTTTTGTCTCTTCTCCCACTCTGTCCAGTTGGGTTTATAATCCCTTTCATTATCTTCTCCTCATTCTCCCCACTCTGTTCTGGTC
TTCATCTGTAGAAATTTATCTGTCACCATTTTGGGTGCTCAGGATGCTATCGTTTCTCAGATCCTTCCTTCGTGTTTCATGGATACC
TCCTGGAACCCTTTTTTCAGTGAGCTGGGAATATGTCTTTCAAGAAAAGGAGGTGGTATATATTATAAAAAATTTAATTTGGGACAA
AGCATCTGACAAAGCGTTTTTCCCTCCTTATGTGTGGACTTACAGCAAGTGTTTTATAATAGCATGCAAAGTAAACCACATTTGATG
GGTTTTATTGAAAATAATACATTTACCCCAAAAGGGGAAAAAGTACATTTTTGTGCATGTCAATTGACAGTATTTGAAATGGAAATT
TTTCTAGAAAATTTGGGAGGTAACAGTCTCAGGAAAGGAAACTTTATATGCTATAATCCAGACATTGGTCACAGAGAGATTTCCTTT
GGTGGCTTAGGTAGGGCAAGGCAAAAGGCCCCTTGTACCCATCTGCTGTAGAGATAGCATATGGAAGAGCCCTGCCACTCCCTGCCA
GTCAAGCCCTTGTGATAAATCTTGATACTCCCATGTATATTTGCCTGCTTGTGCTGCCAAGGCCACTGAAAAAGGTCAGTGCTTTGT
GAAATGGAGAGTATGATATAAACATAGAAGTTGTTAGTAATTTTTATTGTGATTGATCACTTTTTTTTTCTTTTCTTTCCTTTTTTT
TTTGAGACAGAGTTTCGCTCTTGTTGCCCAGGCTGGAGTGCAGTGGTGCCATCTCCACTCACCGCAACCTCTGCCTCCCGGGTTCAA
GCGATTCTCCTGCCTCATCTTCCTGAGTAGCTGGGATTACAGGCGTGTGCCACCACGCCTGGCTAATTTTGTATTTTTAGTAGAGAC
GAGGTTTCTCCATGTTGGTCAGGCTGGTCTTGAACTCCCGACCTCAGGTGATCCTCCCGTCTCGGCCTCCCAAACTGCTGGTATTAC
AGGCGTCAGCCATCGTGCCCGGCCGTGATTGATCACTTTTAAGTACATGAGAATTCTTGGCATGACATACAAAAGAGGATGGGCAGA
GTTCCTTTTCCTGTCTGTGATATATTTCATGACGATCCAGTCTTTGCGGGCAGTGGTTCAGGGTAGTGCAGCCATTGTAACCTAAAC
CATTCCTGTGTGTTTATTTATCAGATTTTCTCAAAGGGTGAGTGCTGATCATTTTAAATTCTGTTTAAAAATAAAATTGTTCATCGA
AGTGATGAAATAAATAGGGGTCAGATATGATTTTAGAGATTTCGAATCCTGAAGAGGGTTTGAGATTTGAATACCATCTCTGTACAC
TCAAGGCAAACTTCCTGTATCACTCAGGACTCATAACTTCATCTGCAATTTTATTATTGGGTGACATAACTGAACAGGTCGCTGCAA
ACACTGAGAATTATTGAGTTGTTTAGGTGAGTCTCTCTTGTTTAAACTGTTGAAAGGTTCATTGATTTGTGGGTGAGGGAAAACTGT
TGTAGCGTAGTTTTAGTGTTTGCATTTCATTTTAACATTACCTTGCAAGGGATTTTTCATGCTGTACTAAGGAGATTACAAGGCCAC
CCTAAGGCATTTTAGGGTTATATAAGGAACTGGCTTTGCTGTTTTATGTACTGGCCCTTTTATTTTCTGGCTAAGCCAGAATCTTGG
TAACCCTCTCACCTATCACCTTTGGGTATTACCTTTTCAGCTCTCCCTTTGCCCTCATGAGTCCAACCAGTGCCCTGGTCTGAGCCT
CCATCCTCCCAACTGCCCTGGCTTCCTGCTGTCTGCCTTCCGTGTTCCTGCCTACGGTTCATGCTTTGCCCTGGACCTCAGTGGTCC
TTCATGTCTGTCTGATCACGACACCTCATGCTATGAGTGACCTCCACAGCTTCTGCTGCTTTTGGTGCAGCATCAAATCCTCCCGGT
CTGTGCTTTCTCCTTCAGGGCCCACCTGCAGCCTTATTCCCTTCTCCTCTTCCTTCTCTTCCACTCCAGTCCTCCTCCCCATCCCCA
GTCAAGTTAACCCTTGGAATATTCTGTCATACCACCCAGGACTTTTCCTTCAGAGCATACATCATAATTAGGTGAGGTCATTATTGC
ATATTTGGGTGATTATTTGATTGTCTGTATCTTGCGCTAGGCAGTCAACTTCCAGAGGGAAGTGGCTGTCCTTTTAATGCAGCATTG
ATGGTAGCACCCACACTCAGGGCAGGGTTTTCGAAGTGCTCCATAGGTCCTGGTTTTCCGTGACAGTGCTGGTATACCCCTGAAGTC
CCCATGTAATTTATTAATAGTGTCCCCTTTCACTTTCAAAGTTACTCTCATTTTGAATGACAGCATATATGATGATCCCTTCTAAAG
CCCAGTGATATGTAGTCATTCAGGTAAAAGGTTTTATTGTCAGGCTCCCAGAGTGGGAGAGCCTGGTTCCAGGGTCGGGGGGTCAGA
GGGGAGCTGTTGCTGAATTTTGATTCTGGCTGAAGATAATTTATCTTCTTGAAACTCTGGCAGCATATACAGATGTGCCTGTTGTTA
ACATTTTAAGAGGTAAGAACACTCTCTGTGCACCTGGGATTTTGGAATATGATAGTGGGAGGCACCAAGACTGTTACAGAAATGGAA
AATGAACACAGGGTGGCAGAGGGTAGGTACTTCTGCCAGTCAGAGGCAGACAGACTTTCAACTGGAAGGGGTCAAGGACACTATTGC
ATTGTGTTTGGCCAAACAAACAAAATGCATTCTAGGGGAGAGCTGGTTTTAGTCGTGTGTTTAGCGGGGGCTCAGTGAGAGGCACAC
AGATGATCTGTGCATCCATTTCTCTACTGATGTAAAGACTCAAAGAAGGTAGTGTGCACGGTGCCAGGGGAATCTTGTTGAATTACT
AACAGTGAAAAACTAAAAAGTGGCCTAAGATCTTGGTTTCTAAGGCCTATTATGGCCGAGTCCCCAGAGACAAAGTTTCCTCCTGTC
TGACTTGGAGCAGATGCTCGTCTGCCTCAGGAAAGGCTCTGAAGAATCCAGGAGAACATGCTTTGCAAGGCACTTTCAAATTGCTGG
GTCCCTTCAGGGGAAAAGGGGTTTTTATGACACCACTAGTCTGAAGATAATTTTTATCAGATCCTATAGCCTGAGGTCTTAAAACAG
AACAACACTAAAGAGCCCCTCTCAGGCCTGGCTTCCTAGCAGCAGTTGGATGGGACCTTGAAGTGGTGGCTCCCTTCATGTTGGCCC
TGCAGGGTCAGGCATCTAAGACTCGTGGAGGCCCAGGCAGTCTGGAGGGATACCAGATGACATAGCAAATGCCAAAAAAAAAAGATC
CAGTGCTATGTTTGCCTGTGTGTGGGTGTGTTTGTTTCACAGGTACAGGTGCACCCACCCGGAGTCAGGGATGGAACATGTGCAGTG
AAGGCTATATGCTTCCCAGATATTGGGGTGAACTTGATGAGAGTCTGGTGGTGGACTCAGGGACCTGGAATTGTCTTCCTCACTTTC
TTGCCACTTCTCAGTGTGAATGTTGTCTGTAGGGGGTGCTCTCCTTGCTTGCCGTGGGTCTCAGAGCTACCAGAGCATTAGAATGTA
GGAGATGCGCAGGCCTTGTGGACGTTTTGCCAGTCGAGCGATTCAGCAAAGTAATTGCAAGGTTTCTCTAATTGTTAAATATCTACA
ACAGGCTGAATTTGGATTGGCTGAAAAGAAAGGTGAAATGAGGTCATCAGAATTACATCATTTTTTTCAAGTTCTGGCATCTTGTGT
TCTTTGCCAAGCAGAAATATTCCAGTAGAACTGTGATAATGCCTTTCATCTATCTGCTGAGCCTTCCAGATGGCAGTTTCTCAGCCA
GTGAGCAAAAGAAAGGGTGTTGAGATTTATGAGTAAGTTGATTCCCTTCGTTTGTACTTCCCAGTGAACTCTCATTCGCTATTTGTG
CTAAGCACCAGTGGCAAGTTATCCTCACATCTTTAGCTCTGCAGGGCCCTTAAGCCAGTAGGCCTTACTTCAAGGGGAGGAGGGCTT
CAAATTCAGAGTAATAAATAGCTGTCTAAATAGAGCCCTTCACATACTTTCTCAGAGAACTTTTTGTGATCTAAGTGGACTTAAGTT
TCTTGAGGACAGGGGCACTCCTTTCTCCTGGGTAGCACCAATGCACAGGAAGCTTGGCGTTGGATTCCGTGGGCCCCTCCCTTTCTA
GTTATGTGCACAGTCTCCCGGGTTGAGAGAATCCCTCCCTTTGCTAAGTCATCTTCATTCAATCCAGGGATGTTTAGGGAATATGCG
TGCCTCGGATGACAATACAAGATGGATTAAGGTGATTCTTGTGCTTTGAGAGCTCTCAGTCCAATGGAGGAAAAACATATAAATAAA
TACACACATAAACGAGAAGTGGTGTATAAGTCTAAAGATCAGGTAATGAGGGAATGTTTTAGAGGAGGTGGTGCTGCTGTATGTATT
GGAGGCGGGTAGGGATTTGCTGAGCAGAACATGTGCCATGTTGTTTTCTCCATCAGGCCTCATGGCTGGAACAGCTGCAGTGCTTTT
CGTTTGGTGCCTCCTCTGCTTTGCCTTTGTCCTGTTCTTGTTCACTTTGAGCACCCTTGCAGCCTACCCTGGGCATAACCCTCCAGG
AACTCTGATGCTGTTTAGTAGATACTCAGAGGTTACCACTTAATCCTTGTTGGTTAGTTTTGTTCCCCAACTCGAGCGTGAATTCAG
GACAGGGCCCATCTCTGACCACACCAAATGCAGTCAGTGCATATGTTAAGATTTAGGGAAAGCGTTTGGGTTGGTTAGTCGAGTATT
TTCATAGATTATTATTACCTGGTTATGTAAGTATGACAAGACAGTTTAACAATGACACAAATATAAGCAGAGGATCTGATTAAAAAT
ACTTTTTTTTGAAAGGGAAGCATAAGTTTGTGGTCAGCAGCAAATAACCCATTTGATCTTCATTCATTTATTCTTCCATTTATTCAC
TCACTAACAGTCTGTACCTTTGTGAATAGCAACAGTCTTTTTCTTCATTGGTGTTTTTTGAATGCTGGGTTTTTACTGCCCTGGGGA
TTTTCTTTAAACCTGATAGTTGTTTTGATAAATCATCTGGATACCAGCTTTGAGCTAATTCTTTTCTATTAAGTACCTCTTAGAGTG
GCTGACCATAAACCATTTGAGAAATCACTACATTTTATTAATGACTTTTGAGATTTAGCAGCAACTTAAATTAATAATACTGGAGTG
TGCTTTGGATAAAAGACCAGGAGACTGCTTCATTCTGCAAACCAGAGCAGAGATGAGAATGTTTGCATTAGGAGAACGTAATTTATC
AGCATTTTAGGCTAGTGTGTCGATTGATACAAGGTTGGGCAATAGTGGTAGGCAGTGTTATAAGTGCTGTTTGTATGTATCAGCTCA
TTTAATCCTCCCAGTAACCCTTTAAGATGTCTGCACTATTATTTTTCACATTATTCTGATGAGGACATCGAGGCACAGAGGGGTTGT
GTGACTTGCCTGCAATCTTGGTTGTGTGCTCAGAAATAGTCTTTTTGAGACTGAAGATATTCTGGTTATGTGTAATACACAGTACAA
CTCAAAGACTGTGAAATAGTTCATAGACAAATTATCCATTCTTTGCCACTTTAGAGTAATTATGCTTATTGTTTCTGTTTCAGGATA
ATATGTGTGTGTTTATAAGCATTTTTTCCTTTTTATTTAATTGCAGTAACATATACATAAATAACTATGATGGCTAGTTTTGAACTC
ATTTAAAAACAAGTAAATTTATGCTTTGATAAGCTAAGGTACATTCAAAGCATTCCCATTTCCAAAGCAAAGTGTTTTCAGTTAAAG
ATTATAGATGTTGCCTTTTGCCAGGGATGCTTGTGAGAATGTCATTTAGAGAAATCTTAAAGTAATTTATCCATAATATTAACTTAG
CATGCCTTCTCTCTGCAATGTACAATGTATCATAGGACAATGTATGATACAGCTCAGTTTTGATATATCAGCAAGTTGAGTAGTAAT
TTTTGTTTGTTTGTTTTAGTTGTTTTAAAAGCTGGTGTTGCAGATTCCTATGCTTAGAAGGACCAGGCAGAAAATAAGCCATTAAGG
GTGCTGGGAATGCAATAGGGAGTGATGGGGAGTGTGGTAAAGGGGTCAGCAATGCTTCTGCCATGCCTATCAGGGAGCTGGTTTCCA
TTAGCTGTTGGTTGACCCAGGAATCTGTACCTGTGTTGCCAGATCTAACCATTTTCCAGGAGAGGCTGGAACTTCATATTTAAAAAA
TGAGAATTTGTCAACTTTAATAATTTGGAAGAAAATTCAAATTAAACCAATACCAGGCAGTGTGGTGTACCCCACATTACAGAGGAA
AGTAGGGGACTTGGGAGGTACATGAGTCAATCTATAGATGCTCCTGTTGTCATGTTAAACTTGTGCTTGCTTTCAGTTGCTCTTAAC
ATTTTGGGTCCACTGTATCTAGACATGTCCTTCAAAAGTTGCATGTAAGTTAATTTTTTTTTCTGTATTTAAAAAATGGCCCCACAA
ACACTAGTCTAGATGTTGAAAAAAACCCTTATACCTTATTATGGAGACTTCTGATGAGTCCTTTTGTACAGTGAACAATCACTTGCT
GTATTTACGTTTGTACTTTTGTGTTTAAAATGGGGAATGGTGTTAGATATTGCATACTGCTTGAGTTAAGGAGGTCGTTAACCAGGA
ATGCTTTGGATTACTCAGATTATTATTGCACTGGTGTTTGTTGTAATGGAATTTGACCTGTCAGGGGAGCTGTAGATGTTTGGATGA
GTGTTCAGTCCTTAATACTGTTATCTAAATTCTAGGCATGTTGCCTGTAACTTCCAGTTTCAAAAAATCTATGTCAATCTGTTCTTT
TATAACCTAGTGCTGTTGTTTTTTTCCCTCTTGTTTTATATTTAATCATTCTCTTTGGCTCACAACAGCAGTCCAGTCCAAAGATTT
ACTTTTTCTATTTTCAACCCCATCATTATTTTTGTGTAAAAAGTATGTTTTGAGTTACAGCAATTCATGGAGATCAAGCTTCTACTC
TGTAGGGAGAAGAGAGATGTCATTTACTACAGAGTTTATACACACAAATGTTCTGCTGCATGCTGTGTTCCCACTGAAATCCAGGGG
GTTTATTTGCATTTATACTTAAAGAAGAATGACCGTATTTGGCTGACTCTAAGCCAAGATGATACAGGCCCAAAGTTTTTCATGGAG
GAATGGGGGAGTAAGGGAAATAGGAACATTTGGAAGAAATAGTTGTTGTTTTTCCCTGTTTTATTCAGTGGCCATTTTATTCACAGA
TCATTTGATGCATTGCATTTACTCGTGTTATTCTGGTTCCTTTTGTGTTTTTTTTTTCCTTTTGAAACAACTTCACACTTACAGGAA
AATGGCAAAAATACTAAAATAAGAACTCCTATAAACCTTCACTGATAAAATCCCTTCAGTTTTCTCCAGTTATCCCAGTAACGTCCT
TTATAGCAAAAGGATCCGGTTCAGGATCACTCATACCCAGCTGTCATATCTTTCCAGTCTCCTTCATTCTAGAAGCATTCTTCATTC
TTCCTTCATCTTTCATGACACTGACACTTTTAATTACAGGCCAGCTGATGTGTAGAATGTCCTTCCTTTTCTTAGGAGATGTTTTCT
GTCCTTCCTTGAAAACCCCACTAGTGTTACCTGCCACGAGGAGAGCTAGGCGCTCTGCCCCCTACCCCAAGCTGGTTGCTCTCTCTT
ACAATGCTTCATTAATCAAGTACCCTTTTTTGGAGCATTGATGATATTCATTCATCCTTTTAGCCAGTAAATATTTTTTGATGTCTA
TTACGTGCCAAATGGGATACATTTAAGAACAAAGCAGACCAAAAAAAAAAAAAAAAAAAAAACCTGCTCTCATGAAGCTTACATTTT
AGTGGAGGGTAACAGATAACCAACAAGAAAAAATAAGTAAAACAGCAAGTACATTAGATCATTAGATAGTGATAAACGCTAAAGAGG
ATCGAATCAGGCAGGAAAAGGGATGGGCAGGTTCTTGATAAAGTGGCCAGGGAAGGCCTCCCACACTAGTGATTGTGGAGCAGGAAC
CCCAAGGAAGGGAAGGAACAGGCTGTGGGGCTAGCTGCAGGAAAGCGGTCCCGGCAGAGGGAGCCTGTAGGGGTGAAGGTTCTGCTG
CACCAGGTGGGCCAGGGGTGAGGAGGAGGCGGTCTGGTGGCTGGAGCCAAGCAGGAGGGGAGAGGAGAGTGGTAGGAAATGAGTCAG
AAAGGCAGAGCAGGGAGATTATTCTAGGCTTGTTGCCACTGAGGGAGATGAGAAACTCTGGGGGAATTTTGAGCAGAGAAGTTACAT
GATCTGACTTATGCCTTAAAAATCCTTTTGTTGTTTACGGAATAAGCAGGTATGTAGGGATGGGGTCAAGGTTAGAAGCAGAGAGAT
CAATTTAAGATCAATTATGGGAGGTTATGATAAGCCCTTTGTGTATCTGTTTGTCCTATTAGCACATAAAGGGGACTCAGTGGTCTT
CAACCTTTAGAATACATAAGATGTACCTGGGCTGTTCGTGAGTGATGCAGATTTCTTCACTGGGAAACCAGCATGTACCTGGGCTGT
TCGTGAGTGATGCAGATTTCTTCACTGGGAAACCAGCATTCATTAGATCTCACCTGTGTTGTCAACAGCCAGCCCCAGGTGATTCTC
ATGTGGATGGTCCTTGAACAACATGTTGATGTTGATAAATATAGTAACAAGTGAGGAATGAGTGAATAATTGGTTTATGGAAAATCC
TTTAGAGATACTTAGTTATGAAGTGTTTCCACTCATTGCTTTATTTCTCATAAACATAGTACTCAACTTAAAAATATGTGGTAGTAT
AAATTTTCTCTTAAAACTTACCTGGCACAGAAAGATCTAATAAGCAAAGTGATTTTTTTTTTTCCAAGAGAAGATGTGTATTTGATG
AAATAAACATACTACTCTGACATACTATTTTCTTGCTTTTAAACGTGGTTTTCAGAAGGAATGAATAAATGCTACTAATAGGGTTCA
TTCATAAGAAGCAATTAAATGTATTCAAGAGGAAATTGGTTTCAGGGAGTAGGTCGGATGAAGCCATTTAAAGATAGTAATAGCAAA
GTGAGTCGTATTTTAGAGTGGACCAGATCTGGCTTGACATTTCATTGCACCAGAGGTTCACAGGAAGGAAGGGGTTTGGCCCTCTGG
CCCTGGAAATATGTAAAGGGCAAAACATAACAGACTTTTGTTTTTTCTCTTTGGTGAAGTAACGTCAATCTTGCATGGGTAATCTTG
AGTTGAACATGAAAATAAGTCTTACCTTGATTAACTGGGTAAAATCTTGGTCAGTTTCATGGGCTTTGGAACCAAAGTGGCTCCAAA
TTCTGCTCGGAACTATCAGTGTGACTGGGCAACTCACATCACCTTCATTTCCTTATAAGCGGGAAATAATACCTGCTTCACAAGCTC
ATTGTAAGGATTGAGAGTTAATCCATTTATGTCTGAGGTTGCAATTTTTTGAATTTTTGCAATCAGACCTTGGCAACCACCTTGAGC
AGAAGGGTATAAAAACTCCCACATGCTTAGCGTTCCAGTAATCGAACACTAGGCATAAATAACTACTGCCCATAGGGCTCTTACAAG
AATTGTCTGGTGTATTATAAGCACTCAACAATTATTAGCTTTTTCTCCTCCTCCCTTTCCTATTATTGTTGTTGTTATTATCATTAT
TATGTTTCAGTGCTTTCAAATCGTATTTTGCCTTTTGGAATCCTACAAATTTTCAATGATGGTTTTTTTGGGATTGGTTGTGTCTAA
AAATACGTTCATTAATGCCTCTCTGTATAGGAGGAATCTAAAGTCTTAAGTGTGGCGTTAGTCTTGTGTATTTATGATCACTTCCTC
GAAGTAGGCTGCCATCACTTATGTAACATTGACTTTCTTTCCCTCTATCAAAGAGAAGCTCAGACTTGCTTCAGGGAAGTATGTTGA
GAATTAATGAATAAGTTAATGTTTGTGGCTCTACCCCATTAACTACTAATTAATGTAAGTTACTGCTCTTATAGTTCTCCAAGTACT
ATCGTCATACTGATTCTCTGAACTAGGGACAGGGACATTGAGAAGACTTTTTCTTATGTACTGTGGGGAAGCAGTGTTTTGTAAAAT
TTTTGCTCTTGCTTTTACAACAGAGTATCACTCCGTATCTAAAATTTTACACCTCCTCTACAAAACAAACAAACAAAAAACACAGAA
AAACCCAACCTAGATTAAATCAAGTAACTTCTTATGGAGCTCTTAGTAATGAAATGTTACTTATGTTAATACGTATTTTTTTTTCCT
CTGGGAAACCATTACTTTCATTCTTGACTTTATCTCCTCATCATTATGCTGCACTTAGTCTGTACTTGGCAATAATGTTCTAAAAGC
TCTCATAACAGTTCCATGAAGGCATAGTGTGGTAGAGGCAGTCCTAGCGTTGTGGTTTGTTTTTTTTTTTAAAGGTTTGTATTTCAA
TTCTGTGTTTCTAAAGGTAAGATTTGGATCTTCTGCCCCTGAACTTTTGTCTGTAAAACAGGGATGGTAATAACTTCTATTTGGGTT
TTTGTAAGGCTTTGGAATAACATATATGTCAAATACCAGGCCTGAATACCAGCAAATGTGGCTGATTGTGACTAATGCAATGATTTT
ATTACTTTTTTTCAATATGATTCAATTTGGTAATATAATTAGAACCAATTTCATAGTTTCATATAAAGCAAAAGAAGGTATTTATAG
GGTCCTAACATCAATCATATTTCCACCTGACATAGGCTTACCTTAAATAACTAATATCCAAACTTACTCTGGGAGGACACCTCTTCT
GTAAATGGATAAAATTGAACTTTTCAAAGTACGTGAATACAGAGGAAACATGGAATTGTATATATGTATGTGTGCATGTGTTGTTTT
CAAATAAGTAACTCACTGTAATGAATTACAGTCAATTTTAAATTTCCCAATTTTGTATTAATGTTGAAAGCCACCAAAGGAGTAAAG
GGATGGGTTGTATCTCAAAAAGGCCACCGGCCTGAGCTAAAACTGTTTCCTATACTCTTCTAAAGGCAATGTTTATTTAATCCCAAG
AGTCCTTTGTTCTCATGATTACAAAGTTAAAATTTAATTATTTGCATTGTAGACTTGAAAGGATTAGCTACACTCGTGATTGATATG
TAAAGAGAGTTTTATAATCTCTGGAACTATGGCTTTTGAGGAACAAAATAATTTAAGCTATAATTTTGAAGTGGGACAGAGGATATT
ATTACAGGTTTATTCATAGGATTCTCATCTTTTGATTGGACTCTAATTAAATACCTTCTTGTCAGCAGGGAGGGTGTGTGGAGGAAT
TATGATTCTCCTGCTTTAATCGTTGTTGATTTCTTAGTGATACTTTGAAGTGGCATCCAACTGTCTTCTCCAGCTTTCAGACCCTTT
GATGAAACAATAGAAACTTGTTAAGTACATGACCAGATTCTTTTCATGCAGTTCCCTTGGGGGAGGTCTATTTTAAATTAGATAGGT
TTGATGTCCAGTCTGAAAAGCATTTATTCACCAGTTCTGATAACAGGTCAAGTAGTTTAACCTCTTACACATAAGACTAAGGGAATT
AAAACCTTTCCTTTCCACCACCACTACCCTCCCCTCCCCTAAAAAAGGAAAGGAATTTTAAAAAGGAAAAATGTAGAAAGAAGTAAA
ATGGTGTTTATCCTTTTTTGTTAAAATGTCACAATTTAGACCATTTACAAATCTGTTTCTTATTACAAAATGATGAGTCTCTTTTGT
CTTTGCTCAGTCTCCTTCCTCTTAGTCCACCAATATTATAATTTTGTTAAATTCTGCTTATCACACTCCTTCCTGCAGACACGTTTT
CTTCTGTCCCACCACTCATTTTCAGAGCTCCTAAAAGTTCAGAAGCCCCTCTTTTCTATACTTTGCACTTTCTCTCATGGAATCTCT
TCCATGCCCATGACCTGCTGAGTCTCAAAGTTTAACTCCAGAGCTGACTGACCTCATTTTTAAGCCTCAGACTGACTGTCCTACCTG
ACATGTCCTCTTGAATGTTTTAAAAGTCATGTCAGACCCATCGGTTCAAAACTGAACTCATGTTCTCTCATGAATGCTGGTTTCTAT
GACTTTTTTTCTAAGCACAACTCTATATCTCATTTGCTTGAAGCCCTCCAGAGAACCCTCAGACATTGCTGATGGGAATGAAAATGG
CGCAACCACTGTGGAAAGTGTTTTGGTGGACCCCCAAAAAAGTTAAAGAATTACCATATGACTCATCAGTTTTACCCCTAGGTATAC
ATTTCCAAAAGAATTGAAAACAGGTGTTCAAATAAAATGTGTGCATGAGGCTGGATGCAGTGGTTTATGCCTGTAATCCCAGCACTT
TGGGGATCTGAGGCGGGTGGATCACTTGAGGCCAGGAGTTCGAGACCAGTCTGGGCAACAGGCTAATTTGTCTCTATGAAAAATACA
GAAATTAGCCAGGCATAGTAGTGGGCACCTGTAATCCCAGCTACTTGTGAGGCTGAGGCACGAGAATCATTTGAACCTGGGAGGCAG
AGGTTGCAGTGAGCCAAGATCGTGCCACTCCATTCCAGCCTGGGCAACACAGTGAGACTCTGTCTCAAAAAAAGAAAGTGTGCATAA
TGTTCAATAGCAGCATTACTCATAATAGCCCCAAAGTAGAAACAGCTCAGATAATTGTCCCTGATGAATGGGTAAACAAATTGTGGT
ATACCCATACAATGGTATAGAAAGGAATGAAGTAATGATACATGCTACAACATGGATGAACGTGGAAAACATTATGCTAAGTGACAA
GCCGTACACAAAAGGCCACATATTGTATGTTTCTATTTATATGAAACGTACAGAATAGGCAAAAATCATAGAGACAGAAAGCAGATT
AATGGTTGCCAGGGGCTGCGGGGGGGGTGGGAATGAGGTCTCCTTTTGGGATATGAAAATGTTCTACAATCAGATACTGGTGCTGGC
TGCACTATATTGTGAATTTACTAAAAGCCACTGAACTGTGCACTTTAAAATGGTGACATCTGGCCGGGCGCAGTGGCTCACGCCTGT
TATCCTAGCACTTTGGGAGCCTGAGGCGGGCGGATCACGAGGTCAGGAGATCGAGAGCATCCTGGTGAACATGGTGAAACCCCGTCT
CTACTAAAAATACAAAAAAATTAGCCGGACGTGGTGGCGGTTGCCTGTAGTCCCAGCTACTCGGGAGGCTGAGGCAGGAGGATGGTA
TGAACCTGGGGGGCAGAGCTTGCAGTAAGCAGAGATCGCGCCACTGCATTCCAGCCTGGGAGACAGAGCGAGACTCTGTCTCAAAAA
AAAAAAAAAAAAAAAAGTGACATATTGGTTCCCTAGGGTTGCTGTAATGAAGTTCTAGAAACTGGGTGGCATAAACTACAGAAATGT
ATTGCCCCACAACTCTGGAGGCTGCAGCTCTGAGATGAAGGTGTCACAGGGTTGGTTTCTACTGATGGTTGTAGGGAGAATCAGCTC
CGTGGCTCTCCCCTAGCTTATAGTGGTTTGCCAGCCATCTTTGTCATTCCGTGGTGTATAAACCTGTCACCTGACCTCTGTCTTCAT
TTTCACATGGTGGTCTCCTTGTGTGTGTGTCTGTGGTCCAAATTTTGTCTTTTTATAAGGACACAGTCATACTGGATTAGGATCTAC
CCTGAACGACCTCTTAATTAAGTATACCTGCAATGATTCTATTTCCAAATAAAGTCACGTTTTGAGATGCTGGTAATGACTTCAATA
TGTGAATTTTGGGGATATGCAGTTCAACCCATAACAGTGACTTTTATGTTACGTGAATTTTCAATTAAAAAAATGTATATAGACAAC
CCCTCCAGGGACTTCTAAGTGCTGTCAAGCACCAGACCAGCAAAGCACCCAATGTGGTCTGACCCCTGCCTACCTTGTCAGCCTTTT
TACTCTCCTCTCACTCTGTGCTGTTTTTTCTGTGCCAGCCACACTCCCTCTGATTGCAGTGCCTGTGCCTACAGCCTGCTTCTCCCT
CTCTTTCCATTAATGGAGGTCTTTTCACCCTTCAGATCGCAGCCCTAATGCTACTTTCATGGAGAGCGCTCGCATGACTTCCTGGCA
CAAGGCAGCCTCTGTTATATGCAGCACCTTTGTTCTTATTGTCGACTGCTGTGTGACAAAGCACTTCTAAACACTGTGGTGTTAAAC
AGCAACCACTCTTAGGCTTGTGGACTCTGCAGGTCAGGAATAGAGACGGCCTAGTGAGGAAGGCTTATCTCTGCTCCATGATGGGAG
AATGTGAACAGCTGGAGGCCAGAATCATAAGGGAGCTTCCTCAGGTGTGTATGCCTGCTGTCTGGACTGGGATGACTCCAAGACCAG
CCTCAGCGGGGACTGTTGACCATGGCTCCTACTGTGGTTTCTTCATGTGGCTTAGACTTCCTCACGACTGGGTGGCCTCCGGGTGGT
CAGACTTCTCACAGGGTAGCTCAGGGTTCCAAGAGTGAGCTAAAGAAGCTGCATGGCCTCTTATGACCTAGCTTTGTTACATAGCGT
CATTTCCAACATGTTCTATTGGTTAAGCTGTCATAAGGCTGCCCATATTCATGAGTGAGGGGACATAGACCTCACCTCTCAGTGGGA
GGAGTTTCAGAATTTGCAGCCATGTTTAAAAACTGCCCAAATTCTCCATGCTACGTCTAAATGTTTGTGTCTCCCAGAATTCATGTG
TTGAAACCTAGTACTCAATGCAATAATATGAGGCTTTTAGGAGGTAATTAGATCCTAAGGGTGAAATTGGATTAGTGCCCTTATAAC
TGAGGCTGGGGAAGCTTGTTTGCCCCTTCCAACACGTGAGGACGCAGTGAGAGTGCACCATCTGAAAAGCAGAGATGAGCCTCGCCA
GACGCTGAATCTGCTGGAGCCTTGATTTTGGACCTCCCCACCTCCAGAACTGTGAGAAATACATTTCTATTGTTTATAAGTTACACA
GTTTATAGTGTTTTTATTATAGCAGCCTGAACAGATGGACACTCGTCAATCTGTGTTCCTTCACGGTGCTCTTTTCTAATTACTCAC
TCCTTTGCATCATTATTTGATTGACTTCTGCCTTTTTAAATAGGCAGTAAGCTTATTTTGCTCACTACAGTATTGCCAGCAGCTAGC
ACAGTAGCTGGCACATAGATGGTTTGTAAGACTTTGAAATAAAAGTTGACTGGGAAGAATTAGAAAAGGTAGAGCAAGGAAAGCCAT
TTGCTTATATCATAAACATCACAAGCTTGTGCAGCTGATCCAAGCATGATCAACAGCAAATATGCCCAGTGGCCTGTAGGTTTATTG
TTTCATGCTCATGATCTCAGTTTAGCAGTAAATCTGAGGCTGCCTTGTGAATATTGATACTGCACGAGATGTCTGAGAGCTGCCACC
CAAGAGACAGTCATATTGACGCCTGCAATAAAATAACAGCAACAGAAGTATAAGCAAAGCCAAATGTCTCCTGAATGAATTTCCCAA
AAGAGATAAGCAGAATTAAAAATTTAAGGTTTAAATAGGCTAATACTGATAGAGTTTGTGTGTGTGCTCGTGGATTTTTATACTTGT
TGGGTGAAGAATGAGAGGTAGTAGGAGATACAGTTTTTTATTTTTAATGACTCACTTTATCAGAGCTTTATGGGGCTGATCATGTTT
ATTTAATACTCTCTGTAGAATTTAGAAAAAGATTATCTTTCCCTTCTCCTTGGCTCCCCAGTTTAAAAAAAAATTTCAAGTCTGAGA
CAGAGGCACGTAAAGCCTTGCCTAGCACACGATTCACTTGTCCCCCGGCTTCTGCACAGCTGCTTTACAGCTATGCTATAGACTTGT
TAGATGAGTGCCAATTTCCCTGTCTTTGCCTGTAATTTTCAGGGCAGTCAGTTACACAGAACCTCTTAAAATGGAAGGTGAATTGCT
GCTTACTAAATTAAAATGCAGTTTTAGTTTTTGCTCCTACACACTGAAGACCCCAAGGCAGTGTGGTATAATATGAAAAACACTGGC
TTTAGAGTCCCGTGGACCTAGGTTTGAACCTTGACTCTGCCCCTTTTATTGATATGACCCTAGGCAAGTTACTTATCCTTCATATGC
CTCCTTTGCTTAAAAAAAGGAGGAGGTAATAACAGAGGTGTTAGAAGAATTTAGTGAATCGTACATATAAAGAGCCTAATGCAGTTT
GCCACGTAGTGAAGCTCAATCAATATTAGTTATTACGACTGCTATTATACAAATGTAATTGCTTGGGGTTTACTTAGAATTTGTATA
TAGTAATACTTGAATGGCTTTCAAAGAATGCTTGATTTTATTCTGGTTTTGCTCTTAATTGGAAAGTAAGTTCACAGTGCCTTAGGT
CTGAGGTATTAGTACATTAATAGTGTGATTATTCCTACTTTGTCTTTGTTTTATACTGGTGACTTTTTTTTTTGAAAAGCATTTATT
AAAGAATAAATATAAATTGAAAGGGGAAATGGTCCAGAATTCTAGGACTTTAGAAAATTGAATGCACTTTTCTACATTTTTCTAAAC
ATCTGTGGGAGTTAAAGCTGCCAGATAGAATGCATGGACCTGTTTGCTTCAGGATGTTTGATTTAAAACAGTGGTTCTTCCCTTTCC
GGACAGGGTCAGAATGACCTGGGTTCTCTCCAAGGTTGTGTACAAGAGCTCCACACCTTCTGTTCAGAAGACCAAGGACAGTGGCAG
ATGCCATCGCCTGTTGTGAAGTGAAGTTGGAGGAGGGAGAATTCTAGAACAGATGGTTTCTTGGATATCTGGGGCCTGTCCAGCTCT
AGCTTTGAAAATGATGGGCCAGACCTTGAACTGGCATGGATACAGGCTTAAGTGCCAGAACAGGAAGTGAGGTCCTAGGGTGATGTC
TTTGGGGCAGCTGCTGCTACTCAGCTGGTGGGCTGGCACCGCTAGCTTTGGCTTCCTATGGTTTGGTGAGGTGATTGTGTGTGGTAC
AATAACAACAGTGAGGTGGGAAACTGGGCCTGCAATCTGAGAAGGCCATAACCACTTTGGAGCTGTTGGCCTATTTTAGAAGGGGTG
TCAGCTACTGTGCCTTGCAGGACATGTGCTACCTTTGGCATGACGGATGCATGAATGTGGGAGCCAAGGATACATGCATCCCCTAAA
CACTGATCAGCCTGAGTTCTGGCTTGTGTAGAGCAGTAAGCAGTGCTTTGTGGGAGGGCTTGGCTGTCCCCTTTGACATTAGCCTCC
AAAGACTAGGAGCTTATCACAGACTTTGCTAACATCCTCTGATACCTTTTTGGAATAATTAAATCATTCTTGGCTAGGTTCTGCTGT
TTGTCCTGCCTCTCACTAGCTGGGTGTCCTTGGGCGAATTACTTAATCTCTCTGTGCCTAGCTTTCTTTATGTATAAAATGAAGAGG
CCAGACTAGATGATACCATAGATGTGAGATGATCTCTGATGTCCAAATCTGATTAAATAATTTTTAGGTATACATGTATTTTGGTAT
TTGCTTTTTTTGAATAGTGAGTCCCACAAATATTACTTCTGTGTAAGTGACACCTACACCCTTTCTTTTGTTCATAAATTCTTTCTT
AAAGATTTCTGTGAGCTCTAATCCCAGTATTTTGGGAGGCCAAGGCAGGTGATTGTTTGAGCTCAGGAGTTCAAGACTAGACTGGGC
AACATGGTGAAACCCAGTCTCTACAAAAAATAAAAAGAATTAGCCAGGCATGGTGGTGCACACTTGTAGTCCCAGCTACTCGGGAGG
TCGAGGTGGGAGGATGGCTTGAGCCCAGTAGGTCAAGGCTGCAGAGCAAAGACTCTGTTTCCAAAAAAAAAAAATTTACGTGGAGCT
TCTCAATATCTAGTATTCAAGAATTTGGAAATTGGAGTGTATCGTTATTTTATGGAGAGAAGGACCCATTAATTCCCTCTCTCGTAA
ATGATCTTGTCCCATTTGTTGTTGGAACAATACTGGTTGCAACTGTTGTTTGAGAGGCAAAGTATGAAAAGCATGGTGTCGAATTAT
AGAAACATATTATTACAGTGGCACTAGAGGCTCTTCATACTAAGAGGAGGAGATTGAGGCCCAAAAATGTTTCAGGGTGAGTCTGAG
GCTGTGCGCTATGCTCCCCTTGAGAGTGGAGGCTGTGCTTTGTTCATCTTGCCATCCAGCGCCTCTTGGGCTGGTTTTGTTTTATTT
TGTTTTGTTTTGTTTTTAAACAGCTCTTGTCAGAGTGACACTAGGTAAAGATCTCTCTTTAACTTCCCCTTGACCTGTTCGATGCTG
AAATGCCGTCACCCATATTTCTAAGCATTATTTCAAGGTAGCACAAACTCCAGGAGCCGTGGGTTTCCTTTTCAGCATGGTCTGTGT
AAACATGGGCTGTTTCTCAGAGGCTGGGCTGAGGCCCCAGAAGTTTTCTTCATAAGGGGTCACCTCTTACCTCTGCCCCTACAATTC
CTTTTCTATGAGGTTTTAATCTCCAAGAGGGAAGCAGAGGTATGGTTCTGAGGAGTGATGTGGATTCTGGGGAATGCAAGGGAAGCA
AGGGCCAAGTGGGTTGAGCTAACGTGGGGACTGAGAAACCATAGTGAGGGAAAAGGACAAGCAGAAGGAAGGGCCTTGTAGAACCAC
CTAGTGCTTGGATAAGATTCTTCTGCAGAGCCCTGGGAAATTGGAAACACTCTCCGGGGTGGTGTGTGCACTGGCAGTTGAATCTTA
GCATCTTCGTTGTTTCCTTTTGATTTCACTTGTTCTGCTTCTTCTCTTGCTGTAGTTGCCACGTGCCCTGACATTTCGCAGAACTTT
GTTGCCATCCATCCAGATTTGGCCATTTCTCCTTTAATTCCACCTGTTGCCCCTCATTGTAAGTGTGACACACTTATCTTCTTTGGT
TTATTCTAAGGCATTAACTAAACATGTCTGAAATTCCGACGAGTCTAAGAATCAATATGTGTGTGTGTATACAAAAAGTAATTCTGC
CTCAGCCTCCTGAGTAGCTGGGATTACAGGCATACATTACCACGCCCTGCTAATTTTGGTATTTTTAGTAGAGACAGGGTTTCGCCA
CGTTGGCCAGGCTGGTCTTGAACTCCTGACCTCAAGTGATCTGCCTGCCTTGGCCTCCCAAAGTGCTAGAATTACAGGCATGAGCCA
CCATGCGCCCGGCCATCAACGTCTGTGTTTAATGCCAGGTTTTTTCTTTTCCCTAAAAGCATTGTTCGGAAATCTCTGCATTTGAGA
ATCTAGAAATAATGATAGTCAAACACGGGACATTTTTAGTGAAGAGAAGCTGAGTGATTGAGAACAAAATGATTGGAAAGAAGGGGC
GATGACAACCTTAAAGGTGGGAACAATTTGCTAAGAGAGAGGGAAAAGTACTGTATTGTCATTGTCCCTGAGGTTTCCAGGCTTGTG
TGAATTTTGAACAGGGGCAGCCTGTCCAGTTGTAGGAAGGTTGTGGCATTAGGCTCTCTTGGTCCCAAGGCACAAAAACTCTGTAGC
AGGGGTTTTCATAGCAGGTTACACGTGGCAAGGCAGATGGGAATCTTGAGAATATCTGAGGATATGAACTGTGGCTGGGCCACACAG
GTATGGGCTGTCACGTGGTTCCCGATCCAAGGCAGCACTGGGGGGTTGGTGACTCTTTCCTCCTTGAAATTTCTGCTACGGTGACTG
TGATGATCCTAGCTAGTTGGCATCATCTCTGTTGAAGTAGACACTTGAATTAAATATGTTGCCCTATGTTGTTGGCCAATAGGGCAT
AGTGTATATATCAGGGAGGATGGCAGCACCTGTTGGCTGTGATGGCATTTAGCTTGGGCCTGACATTCATGAAGGGCCTCAGATTTA
TTACTTCATGTTGTCTCCACATGAACTTGTACGTTTAGTCACAGAGACAAACTGGCAGGATTGAAAGAAGATGGCATTCGTCATCCA
GATTTAAATATGTTTCTCATTAATAGACATTCTGCCTGTAGTGCACTATGAATTAATACATTTTATAAATCTTGTAACTGCCTTATG
AAGAGCATAATTAGTTATAAGTGTTTGGATGTTAGAAATGTTAATTTGTTCAATATAGGCGTTAAAATGTTTTGTCATTTTCTTATT
TTTAATGTTATTGGGAACTTTAAAAAACAAGAGTGGCTCAAGCCCCTCTTGACCTCTTGGAAGGCCCTGCTGAGCTGGGTTTGCATC
GGCTGTTCTGGGTCATATGTTCACTTTGGGTCCAGTTGGCTGGGCCTAGGGTTTCACAAGGTGTGGAAACCTTTGTCCCCCATATGT
AAAATAGCCTGGCTCCATTTCTGTCAGAAATGCAGCCCACTCTACCCGCCACAGCCCAGGCAAGGGGATGGAGCTGGGTCAGTGGGT
GTGACAGGCTGATGACGGAATTGAAGAGGGGGAGCCTGTGAGTCTGACTTACGGGACTAAAGAATAAGGCCCCAGGCAGAATTCTTG
ATTTACACATAAGACTCCAAAGTTTTAGGCAGTTCATTTCAGTTTTCTGCATCTACACTTATAAACTACAGATGAATAACACCAAAT
GACTTTTGAAAACTCTGAAGACTGTAAAACTCATTTGTGCTTTGAGATACCAAGCCACTGCTAAACTACTTTCTTGAAAATGAAATC
ATTGTTTAGGCAGTTGGTGGTTGTTTGGAAAAATTTCAGAGTGAAGGGCTCTAGGAGAAAAAGAGTTCCAGAAATAAGGAGAGGATA
AAGTGATGCAAGGGACCATTTAGCCGGCTAGATAAAGGCTGGAAGTGATAATTGGTTGTGCTTAGGGAGGGATAAAATTGGAAAGCA
ATGTCAAAATGCAGCGTGAGTCATTTTGGGGGGGGAATCTTTGAAATGAAAAACTGGTATTTAGTAGTAACAGCGACCATGCATTTT
AAAATCCATCAACCAGCATCTCTATTCCCCACCCTCAATATCAACCTGTAATAATTTATGCTGTAACCACCACCGAGAGATAGCTTA
GGACTGGATTACATCAGTCACTTTTTTTTTTCTTGGTTGTATGGCAGATATTTTGATGTTTACAATAGGGACTAGAGAATTTTATGG
GGAAAGGTTGGCATATTAACAGTCATTGAAGTAAGCAGTCTGGGACCCTGTTGTGTTATAAGGGAAGGGATTTTGGACCTTGGTGTT
TTGCAGAGGCCATATTTGCATAATTAGAGAGCACAGGATTATGGGTAAAATGGTTGCAACTACTAAACTAGCCTAGTTTATATGGAT
TCTTGTTCATTTGCAAAATACCTTTTCTGTGTGCTTGTTGAAACCATAATATCCTGGGTAGGTTTTATTACAATAACTTATGGTCTA
GTAACATATCTGGAATACCATAAGAGAAAGCATAGGCAACATTTGTAAGCCTGCTGCTTTATGAAGAGATTGTGCTTATCAGAAAAA
TTGTAGCATTACTGTAAATTTTTTCTTTCATATGGAGTGATTTACTTGGTATTTCAGTATATTAATTTGCTAGGAAAATGTCAAGTT
AGTACTTCTCTCAAAAGGGCATTGTGCATCTGAATCACCCGTGGTTTTTTTTTTTTTTTTTTTTTGGTTTTGCCCTCAACCCAGATG
TGTTGTGACTTAATCTCCAGTTCTGGGCCTGGGCACCCATCTAAAATGGTGAAGAGGACAGAAATGATTCTTGACTTCTTGGTGTGT
ACGTCTGTGAGGAACCCAGATGATAACACTGCAGGGGGAACCATAATTGTAATTGTGATAAGTGCTACGGTGAAAAAAAAAAAAAAT
AACACGGGGTAAGAGACTGGCAGAAAGAATGTAGTTTTGATGGGCGGGAAGGCCGCTCTGAGGAAGTGAGACCGCAGCTGAAAACTC
AGGGATGGAGAGGTTAGCCCTGTGGGGGGAGGGAGGAAGGAGCATTTCAGGCTGAGGGGACAGAGTAAGGGAGGCCTGATGTGGAGA
GGGCTTGGTGGGTTTGAAGAGCTGAGATGATTGGGACTTGTGGCCAGAGATAGGGGAGCGGGGGGAGCCCCGCGGGGAGCGGGAGAG
GATGGTGCTCCTGGCAGAGATGGCAGGTGGCCTGCTGTTTCTATTCTGTGCTCCTTAGTGTCTTGGTTATGAAATGACTTGCCAAAA
TGTCCCTTTAAAAGAAAAGGGGTCCTTGCCAGTTACAGCAGTGTCACTGTTTAGGTCTGCTAAGCAAATTCTAGGGTGTCTGATGGC
TTTGTGTTGGGAGTTGTAAATGGATTTCATTTGAGAACATGACAAGAACTGAAAGATTAGTTTAAGCAAAAGAATTTAATTGAAGGA
TACATCATTGTTCTTGTAAATCCTTTGATATTTGGCTGTTGTCTTCCAGCCTTTTCATTCCCTGCATCCTGTCTGCTGCTTGCAGGA
CTGGGCTGTAAGCCTTCATTCACCTACAACCCTTTTGTATTGCTTGGAACTTGCTTGCTTCTTCCTCCTGCTCCTCTTGTTCCCCAC
TCCTCTTCTCCCTTCACCTCCCCATCCTCCTCCCCCTCCTCTTCCTCCTGCTTCTCATCTCTTCAGATGGAAGAGAGATGGGAAAGA
GCATTCACTTAGTTATTCTTTGGAATAAAATATACGTCAGTTTCCAGCGAGGGAATTCTGGCAGATTTACTGACAGGTGACAGTGTC
TGTGTAGAATTAGGATTCCTAGGAAACATTAGTTCTAACTACATTATTGATAACAATGCCATGTGCATTGTAAATTTGGGACCAGGC
CCTGCATGCAGTTGTGTGGTGGTTCCCTACACAAGGGCTCCTGGCTGAAGGGCAAATGAGGGTGGAATCCACTGAAATGCAGCCCAC
TCGCCACCCTCCACGGCCCAGGCCAGGGGATGGAACTGGGTCAGTCTGGAGGAAGAGGGTGTTCTTTTCTGTTTCTTACGCAAGCAA
ATCACCAGGCCCATGTCTTGACTCCTTCAGAGGTGCCAAAAGGGCTGGTGATAGCCCTGCCTGGGACAAACCTCAGTGACTTCTGGG
TCGCACATTCTTGCCTGGGAAGACCCTGTTCTTACCTGGTAAGGCACAATTGAAACTATGATTGGTCCACTTCACAGACGAGGCAGC
TTGTTTTCTCCCCTGGTGTGTTATTAGTTTTAATGGACATATTTGTGCAATCAGTGTGTACAGTTCATGTTGGTAAGCATCTGGCAT
ATTTTCATTATTTTAGAATAATGCTATGCTGAGCTCACATTTCAGCTTCTTGTCAAAAATAGTAATATTCTGGTATGGCAGATTTCC
TTCTGGGGCAGTACTTCCCACCAGTGTGTGCTGCCTGGATATAGTTTCATTCACAGAGGCTGCTTCACATCACATTTCAATGCATTT
GTGCTTTAATAGTTGATTCTTAGTATTAATAAATTGACCGGGTGCTTTCATCTCCAGTTTATAGGAGGAAATTGTGCAAGGAAAAGG
AGGCTAACTCACTTGCCCTAGACAAATTAGAGGCTTGTTTCAGGGTTGGAAAACAGGTTCTCATATTCCTTCTGGAATATGTTAAGG
TGTGGACTAATCTCTTCTGTGACAAGGACATCCCTGAGTAGTACAGTGTGTCCTCAGATCGTAGTCCAGAGTTAAGAAGTCAGGGTT
GGAGGAGTTCCTCTCCCTTCCTCCATGCCTGGCTGTCCAGATGGCTCCGTTTGTCACCATGTCTTAGCCAGTGGTGCAAGTGGCTGT
GTCTTTAAGAGGGTGACCTCAAGTTGGACACATTACATCGAGTCATATCACATCACGGTATAGTCACAGGAACCCCCCCTAGTTCCC
AGGGAGCCTAGGAAATATAGTCTATACCTGGGGAGCGGGGTCAGGGGGAATCACATGCCTGACTAGAGCTAGGGGAGTTCAGTACTA
AAAAGAGTAGCGGGGAGGATGCCGGGGCACTGACTACCACATTAGTCCTCGTTCCTCCCTGAACAGCCTTAGGGTGGGTGAGCAGCC
CTAAGTGAGTTGTTAGCTAACAGAGATGTATAATGAAGGGGCACCACAGAGCATTTTATATTCCTACTGTAGCCCTAAATGAATAGG
CAGCTCTTTGTACATACGCTTGAATATGCTTCTTGGAACCAGTTCATTCACTCGACAGATACGTGCTGAGCGCCAGTTCTTTGCCTG
GGTGCTGTGTGGTCTAGTAATTGAAGGTACCTGTAAGATGGCCAGGGCCAGCCCTGCTCATCATGAAGGGCCTACTTATGTAGATAA
TTTTGAACCTCAAATAGGAGGGAAGTTGGAGTTGATTTACCTGTCATTAAGTTGTTAAAGAAAGATTTTTCCTACTGGTAGCAACTG
AATGGAGCTCTACTATGGTTTGAATGTGTCCCCCAAAGAGCATATATTGGAAACTTAGTCCCCAGTGTAACCGTGATGGGAGGTGGA
GCCTAATGGGAGGTGTTTAGGTCATCAGGGCTCCCCCTCATGAGGGGATTAGTGCTGATTATAAAAGGGCTTGAGGCTGAGTTCGAT
CTCTTGCTGCCCCTCATCTTTTGCATTCCACTATGGGATGATGCAACAAGAAGGCCCTTTGCAAATGCCAGCTCCTCGGTATTGTGT
TTCCTAGCCTTCGTAACTGTGAGAAATGAGTTTATTTCTTTACAAATTACCCAGTCTGTGGTATTCTGTCATAGTGACACAGAATGG
ACTAAGATGAGCTCAGAGGGAGAGAGGCTGTCTAGGCCCGGGGCCTTTGCTGTACAGCAGCTGGGCTAATGCCTCTGCGAAATTATA
TTAGCCTGTTTGTGTTAAATGAGGAGCCCTCCTGCTTGAAAGAGGCAAGGCATTCTGCTTATGAAGCCAGAGAAATCAGTCAGGCAG
CCTCTGACTTACCTTTGCCCTCCCCTTCTGCTCAGAATCCTTCAACTCACTGTCCGTCAGGCTCAGCCCTGTCTCCCCTGGTCTCTC
CCAGACCTGCCTTTCTGGGTGTGGGGTCTTCTTGCCTCCCTAGGTAAGGGACAGATAATGCCTTGACCACCCAGGGAACAGGAGTTT
GGGACATTACCCCTGAACTGAGAGGTGGTGCTGATGAGAGTAATTAAGCACCCAGAGACATTGTCCTTCAATACATTGCAAAGGAAA
GCTTGCTCAGAGTAGACCTGCACCTGGACACGAGAGGCTGGAGACTGTATTCAGGACAGTGCTCGTGGGGAGCTTCCTTCACTGCTC
CACTCTTGTGTCCTGTGTTACAGGGGCTGCTGGCTGTTGCCACTGTTTTTCAGAGAATGGGCTGCCTGTACACACCTGACTTGGGTG
ATCATGTCTACCCAGGAGGACCAGCAAAAAGGGACAAGCAGTGATGCCATCTGGTTTTACCTCCAGCATTCCGAGCACTGCCCTGGC
CCTCTCCCCCAGCCAGTGGTCGGGGTTGGGTGCGCCCCACTCTGGCTGCACAATAGAGTAACGCAGGAGCTTTAAAAACTCCCAACA
CCCAGAGCTCACCCCCAGCAAATCCGACCTAAATAGTCAGGCATGGGGCTTGGACAGCAGTATGTGTATATATTTTAAGGTTTCTTA
GTTGAATTTAAATGTTACAGTGGGGATTGAAAATCTTGGTCTAGTGGTGGTAGCTGTTGGGTTTTTTTTCTTTTCATTTTTTTTGCA
TCCATTCCTGTTACTACCCTCTAGACCACTGTGATTTCTTAGTCTAATCCTGACTTGCTACCTGAAACCCTCTAAATGTAAGTCTCG
TGCAAGAGAGTGAGAGGTAAAGTTTGGTTTTTATGATAAGAGATACCTTGCTCAGTAAGCAGAAATAAGTGCCATGTGTTAGTAACT
ACAGTTTTGTTTTGTTTTGTTTCCTGTGGGAGCGACAGAAAACAGTTTACAGGGTGTGGTGGGGAAAAGAAGAAACGAACATCTCTC
TGAGGGCGAGTATAAGGTTGTGGACTTAGTGGGAAAAGTCCTCAACTTCAAGTCAGAGAGAACTGTCGAGGAGAGAGACTCATGAAG
AAGACACAGTGAGGTTTTTAGTGGTTAAAAACACAGCACTGCGTTTACATAGTCTATTTCCCCCCTAACAGTGGCAACATGTTGTGG
ATTTTCTTTTTTTTTCTTTTTCCTGGAGAGAAGCATGCTGCCTCTGAACAGTGGAGAGGAGGAAAATCATCAGCATCCTCATTGGGG
TCAGGTGACCTGGGCCTCGTTCTTCCGCTGCCACCTGCCAGGGTTGGGATTGTCACAGAAGACTGTTCCTCTCCGTGGTGACTGATT
GCGGAGAGATAAGGGGGTTAGGAACATGCAGCTTGCTGTGCTGGGCTCAGAAGGGACTCCATGTGATTGACTCCAGTGGAGACTTAT
TTTACCTGCCATTTCTGTAGCAAGAAGGAACCCAGATGGGGCCAGGGGTCTGTGAATGCCTGGAAAGTATATGTCAAACATTGTGTG
AGGGTACACATCCTTTTCTGGGGAAGAAGTTCCATGGCTTTTAAAAATCAGAGTCCTAAAGGATCCCAGTGACTAAAAAAAGGAAGT
GAAGAATTCCTATCATAAGTATCTTTATCTGTGATAGGGAACGGGAATGTCTGCCTGATGTAGTCTAGAATCCTGTGAATTCAAACA
GATACTTTGTTCGTTCACACTCCCGCAGCTTATGTCTAGCGTGTTATGCTATAACGAATAGGGTTCTAACATTAAACTTATTTTTTG
AGAAATCTTCAGGAAAAAATACCATCTTATGAATAGGTATAGTATATATAAATTTCATGGCCCGGCGCAGTGGCTCATGCCTGTAAT
CCCAGCACTTTGGGAGCCCGAGGCAGGCGGATCACTTGCGGTCAGGAGTTTGAGACCAGCCTGGGCAATATGGTGAAACTCCCTCTC
TACTAAATACAAACATTAGCTGGGCATGGTGGCGGGCACTTGTAATCCCAGCTACTCAGGAGGCTGAAAGGCAGGAGAATCGCTTGA
ACCTGGGAGGCGGAGGTTGCAGTGAGCTGAGATGGCGCCATTGCACTCCAGCCTGGGTGACAAGAGTGAGACTCCATCTCAAAAAAA
AAAAAAAATTTCATTTAAGGCATCACAGATTGTTTCTTTTTTTTCCCAACTGTGTACTCTAAATACAGGCAAAAAGCATGTAAGGAA
GGTTGTAACAAAATGTATATGTAGGCAATAGGACTGATAAATACAAATAAATCACAAAAACTATATGGAGGAAGGGGGAAAATCATG
CCAAATAACTTAGGCTACTGAGGTTATTTGAGCATTACATTTATAATGAGCTTCCTGGAAACCAAAGTAAAAAGAGAAACACAGTGG
ATGTTAGAATTTCTACTTAAAGATAGCAAATTCTTTCGTTAGGTAAGACAGTCTTTCTGGGGTTCATGTCTAAGACTTTATCACATG
TCGCATTCCTCAACACTTTCACCAAACATACAGAAATGCCTCTCTTGTGATTGCTTTCCACTTCCACAAAACTAGTTGTGTGGGATA
CCCATCTCCGGATGTGTGGTTCTTTGATGAACTCTCTCCCCTGTTTTTCTAAAAACTCAGTTACCTTAGCTGCTGGGAATCGCAGTG
CAAGATTACACTCTGTGGAACTGCCCAGAGCGGGGAAAATGAAGAGGGTGACAGGTGCTTTCCTTCAGGAAATGACAACTTAACAAG
CACAGTGATGGTTTTTTGCTATGGAAATGGAGGATGCTGACCAGAATGTCTGCTGGGATGGCAGGTCCCCCGGCTGCTCCCTTCCCC
CCACTGCCTCACCACCTCCCCCTGCCACACCACCCCACTATGGGGAGTTGAGCTGAGGAGGCCCTTTGCAGCCATCCGTCTGCACCT
CGCCTCTTTAGGAAGTACTCTTTCTTTCACCTTCTTGCACATAATTTACTTTGTGACTACTCATAAATGCCCCCTAAAGATGAAAAC
ATATTTTTAAAGTTGGAGTGAGAATCACCGTAAAGCCTTTATAGAACCAAATGACGTTTCTTTACCAAACTGCAGAGCTTTCTTTTC
TGCTCCTTTTCTTCAAAAAGAGTCACAAAAATTCAGGAATTTTAAAAACCTCTGCATTTAATTCCTCATTGCCTTTCATATATGTTC
AATAATTGCATAAGCTGTCTCACTTAAGCTGTTGCTAATAATAGCTTTTTTGTCGTTTTGGAAAACTATAACCAAGAACCCTAATAC
ATAAATACTTCAAAGTATGTTTTCTTAATAATGGTGTTTGATGTAGGGTGGAGGATGAGGGGAAGTCAAATATTTAGACACTTACAT
AGTTTATGTTTAAGAATTATCTACCTGTATATCCATTATAGTTTTATCCAGAAATAGCATGTAGTGTGGAAAGATGTTTCCGTAACC
TTCCTGGAGTAGCTCATAAAGCAAACCTAAGAATGCTTTGGAAGTCTTTATGGTATGAGTGTTTTGTGTTTGTCCTGTTTTCTTTTG
AAAAGTACAGATCCAAGGGCAAACCTTTCAGAATGGGGTAAGACAACAATTGAACTTGATGAGTTGCCTTATGTAGCAAAGATGGCT
TCAAAGATAAAGGTAATCTGCAGAAATAAAATCAAGCTTCTTTTGCTCTGTGACTGCATCACATGAAAATGGCTAGGTAGATTTTCA
CTCGATTGGAGTTTGTTTAGGGTGGTCTGATTTAAAATGTAGGCTCTTGTGATGTGCATAATTTTAGATGGTCTTGATTGCCCAGAG
CAGCTGTTACTGCTGTGCAGAAACAAGTGTTCAGTGTCCCTTTTGGAGAAATGTGCCGTGTGCATACAGATGCCATAGACAGAGAAA
ACAAATGGTGGTGTCAACATGAGCCCCACCTCAGGTAACAAGGACAAGCTGGATTCCAGAGGCAGGGTTGTGTGGGGACAACTCCGT
GAAGTCCACACAGCAGAGAGGAGCTACAGGGAGGTGTTTATTTCACAATGGTTCGTGATTTTTTTGACCAATGCTGGATGGGCAGCT
AAGATATAGTATCAAAAACACATTAACTATAAATGGGTGAATTTTAAATAGCCACATGTACACCTCCATTTTTAGACTGTTGAGAAG
CCATTAAAAATTAGTTCCTCCTGTGATTATTCACAGGGATGAAGTATTCCGAGGGATTGACAGTTTGTGGCTAAGCCATTTAAAAGG
CTGTATAAACTGGGTATTTTAAATTAGTCTTATAAGTGAACTTCAGTATACTATGGAGTATTTTTCTTAAAAATTGTATGCTTGTCA
TCTTTTGGACATTCTTTGGGTCAGAGACTCAAGTGATTCATGCAGGAGGTCACAGTGAGTAGTGACTGCTGAATTAGGAGGTAGGGC
AGGGCAGAGGTATGTCTCTGCTCTTCTCAGCTGACCGGTCGTGTGTTGGTTACTTTGTCCACCAAACAGTTCTGGCACCAACTTTGG
AAGGGTGATGCGATATTATGCTCAGTGAACTGATTAGTGCCCCCACAATTGAACCTTAGCCAAGTTGTTGGTTGATAATTGGCACAT
TTTTTCTGATATGCTGATAATCCAGTTACTGCTTCAAGTTTATATTAAAATGTTTGACTCTTTCAAACCTTGCGGTTTTTGATGATG
TCAGTAGGTCACCAGACTTTCTGAAAATAGTTCTTTGTTTCAACAACAACAACAACAAAAAAGAGAAAAGAGATAACACATAAGCAA
TAACTTACCATTATTGTTTTTTAAACCTTCACAAACTGACTTCTTTCATGGAAATACTGAAATATTTATTTTGTATTTAATTTATTA
TATAATTTAAAATGTAGAGCGAGTGAATTCAGTTTGGGATCTACTGAGTCACAGTGCCTGGTAGCAGTTGGATGTTGTAAGAGTGAA
GTCTGAGTGGGAGAAAAGGGTTGAGGAATCTCAGCTGAGTGTCCCTGAGCAAGCTGCATAACATCTCCAAGCCTTGGTTTTCACATC
TGACAAATGGCATGAGACCTGCTCTTTAGGGTTGTATTAGAGATTAAATAGTCACATAAGGAGTACTGAGACTGTAGTATGTGCTCA
GTAAATATTCTCTTTTCTCTCTCCCTCTTTTTGGGTATGAAGGTAGTTGTAGTTGTAAAATTTTCTTCCACACTTAGGAACAGATAT
TTCTCTAAATGTAAGCCCCCTGGGCTTATTCTAGTTTGTTCGTACATCTTAAAAATGGAAACCTCAGACTAAATATAGTATTCAAGT
ATAAATCCCACTACTGCCTAGATAATGGTAAAGACATTACTTCATAGTCTTTGCGTGCCTTGTTCCCATTAATACATATCATGTCAT
GTTAGCTGTTTTATAAGACAGTTCATTGCTGATTCATCTCACCAATTCAGTTCCAGAACAGCACCGTGTCAAGGATGCAGTGGCTCT
GTCATTTTGATGACGAGATGTTAGAACAGTTTTCAGTGTAAGAATCTGCTTAGCATTACAAAGCATTCATTTCTGTGGAAAGAAGTC
TTCACAAAGCCCGGGAATTGACTAAAATAACCACCCCTTTTTTAGGGGTTTGGTGTAAAGAATAGAACATATTTGAAGCCTGGGTGA
TTGGTGATGGGGAAAGTTACATGAACTGAGTTTTGGCTTCATTTCCCGGAGTGATGATAATGAGTTTACTGGGTGCTGCAGTACTAC
GAGATCACAGAGTGCCAAGACCTCAGTGCAGTGCTGCGTGATTGAAATGACTGATACTCTCTTTTTTTTTTCATGCTAGTTCAGCCT
GTTTTCATACACTCCAGAGGTTCAGTAACCATCAAACAGGGAAAAGGAAATGGCAGTCCAATAGAGCGGTCGTTCAAAATGACCAAA
GTAATTCCAGAAATTCAAGAAAGTAGTTGTTTCTCAAGTGTCCATTAGGATCGGGCTGTCTTAGAGTCTGCATAACTCGGAAGTGAT
TAGAGGGGTTTTGTGGCTTTTCTGTTCTCTGATGACTGGAGCTGGTTTCAGCCTTCAGAGTAAGTAGCCAGAGAGTAAGATAAGAAA
TGCTGAGGAGCTGAAGTGGGCACCAATTTTATTTTCCATTGTGAAGGAGATGAGCATCTGTAACCCACAGCAAGATCAACCACCACA
TTAAATTTATATTGTTTGAGACAGTTCTGATCCACTTGAGTGTGTGTGTTCCACTAACACTCTGAGATGGTTGCATAGGCCACAACT
GTTCTGTCCAGTTCCTGCCTGATAGGTAAGCTCTCTGGGTAACTTTATTCCCATGAATTGAGATAGCAGAAGACAAACACCACAGTG
GCATTGGTGTATCCAAGGCACTGATTTTGTTTTTCTTCCATGCGCTAGTTTGAGCTGGCCAACTAATCATGGTATTAGCCTCAAAAT
GTATTGACAACAGATTTGTTTTGATAGCAAAATTGCTTTTATACTTGAGAAATCTGATCTTTTATAAAAGGCCCAGGGTAAATGTCC
TTTAGCAACATCATGAACATGTGTGATATCAGTGATATGGTGGGGAGATGTTCCTCAGCAAAGGATGTCTGTATTTGTTCCTAGGAT
TGACAAAGCTGTAAAGCAAAGTGCCTGGTAGGGGGGAGTCTAAAGGAACTAGTAGGTAAAATGATTAGAGAGCTTACTGTAGTACAG
ATGGCGATATGCAGTCTACAAATGACATCTCCTTGCCACCACTGTTACTATGGGTTCTAATGAAAAGAACCTGAGACTTTTGAGAAT
TTTTGAGTTCAAAGGTATAGAAGAAGGAAAGAAGCTGTGAAAAGCAGGCATAACATTGAGGGACAGAGAATTTGGCTAGGTAGGTCT
AGACTGTAAATACAGTAAACTTCCCACACTACAGTGGTAAAGTAAAATTTTCCCCACTTTACAGCATACAGCCACCTTCTCGCCATA
TTCCAAACTTTTTCATTGCTATTATACCTGGTATTATTTCACTTCACTTTATTGCATTGCTCTATGTCATATGTGTTAGGAGCCTGT
TTGGAAGAAGAGCAGCATTGTAGAATTATGGAGACAGCCATCTCTTGCACCCGTGTTTGAGCATCTTCATTTAAAGGGGTTACCAGT
CCCTTGCATAAATCTCTATTTAATCAGCTTGCTTTCCTAATCTTCTGCATGAGCAATTCTGTCGCAGGGCAAAACCGTTGATTGTAG
GGGAAGAAGAGAGGGAAAGGAGAAAAATGTAGTTTTGGAAACAGAACACTTCAAATGAGGGTAGGCCAGGTTCTGTAGCTTCCTCAG
GGATTTAGAGTAGGGTTGATATGAGAGCTGTGAGTACCTAGGCATCTGTGGAGAAGAGGGAGTGGAAGAGAGGTAAGTGAGGCTTTG
TCAAAAAGAAATTTTGAAATGAGTAAAGTGTTTCTGGTTTTGCCAAAAAAGGAGAGGAAATTCTCAATAACCTTCTTATACCACAGG
CTTCCTCCTTATCAGACCAATAGTGATCGTATCAGACATGCTGCAGATTAAAAAGACATTTGTGGGTCAACCTTTAATTTTTACCAT
TAATCACAGGAATGCTTTGTTTTATTGTGCTTCACTTTATTGCTCTTCACATATATTATGTTTTTGGTTTTTGGGTTTTTGCTTCCT
AACAAATGGAAGGTTTGTGGCAACCCTTCATGGAGCAAGAGTATCAGTGCCATTTTTCCAATCGCATGTGCTCACTTCATGTCTCTG
TGTCACATTTTGGTAATTCTCACGACATTCCATACTTTTTCATTGTTACTATATCTGTTATGGTGATCTGTGATCAGTGATCTTTGA
TGTTACTATTGTAATTGTTTTGGTGTGACATGAACCACGCCCATGTAAGAAGGCAAACTTAATAAATGTGTGTGTTCTCACTGCTCC
ACCGACTGGCCATTCTTGTCTCTCCTCCTCAGGCCTCCGTATTCCCTGAGACACAACAATATTGAAATTAGGCTGATGAGTAATGGT
TTCTCAGTGTTTAAGTGAAAGGAAGAGTCACATGTCTCTCACTTTAAATCAAAAGCTACAAATGATTAAGCTTAGTGAGGAAGGCAT
GGTGAGAACTGAGATAGACCAAAAAACTTGGGCCAAACCATTAGCCAAGTTGTGAATACAAAGGAAAAGTTATTGAAGGAAATTGAA
AGTGCTATTCCAGTGAACCCATGAATGATAAGAAAGCAAAACAGTCTTATTGCTGATATGGAGAAAGTTTTAAATAGAAGATCAAAC
CAGCCACAACACTCCTTAAACAAGAGCCTAATCCAGAGCAAGGCTCTAACTGTCTTCAATTCTGTGAAGGTTGAGAGAGGAGAAAAA
AACTACAGAAGAAAAGCCGGAGTCTAGCCAAAGTTGGTTCATGAGGTTTAAGGAAAGCAGCCATCTTCATAACATAAAAGCGCAAGG
TGAAGCAGCACGTGCTCATGTAGAAGCTTCAGCAAGTTATCCAGAAGATCTAGCTAAGATAATTGATGAAGGTGGCTACACTAAACA
ACAAATTTCTGATGTAGATGAAACAGCCTGCTATTGGAAGAAGATGCCATCCAAGACTTTCATAGCTAGAGAGGAGAAGTCAGTGCC
TGGCTTCAAATGACAGGCTGACTCCCTTGTTAGGGGCTAATGCAGCTGGTGTCTTTAAGTTGAAGCCAATGATCATTTGCCATTCCG
AAAATCCTAGGGCCTTTAAGAATGATGCTGACTCTATAAATGGAACAACAAAGCCCAGATGACAGCACATCTGTTTATAGCCTGGTT
TACTGAATATTTTAAGGCCACTGTTGAGACTTATTGCTTATAAAAAAGATCCTTTCCAAAATATTACTGTTCCAGTGTGATAATGTA
CCTGGTTACCCAAGAGCTCTTATAGTACAAGGAGATTAATTTGCTTTCATGCTTGCTAACAAAACATCCATTCTGTAGTCCATGAAT
CAAGGAGGAATTTGACTTTCAAGTCTTATTATTTAAGAAATACATTTTCTAATGCTGCCATAGACTATATAGTGATTCCTCTGATTG
ATATGGGCAAAGGAAATTGAAAACCAGGAAAGAAGTCATCATTCTAGATGCCATTAAGGTCCTTCATGATCCATGGGAGGAGGTACA
AATATAAATATTAACTGGTGTTGGGGAGAAGTTGATTCCAACCCTCATGGATGTCTTTGAGGGATCCAAGACTTTAGTGGATGTGGT
ACAGCAGATGTGATACAGATAGCAAGAGAACTAGAATTAGAAGTGGAACCTGAAGATGGGCCTGATTGCCACAATCTCATGATAAAA
CTTGAATGGATGAGGAGTTGCATCTTATGGAGGAACAAAGAAAGTGGTTTCCTGAGATGAAGTCTACTCCTGGTGAAGATGCTGTGA
ACATTGTTGAAACGACAGCAAAGGATTTAGAATATTCCATAAACTTAGTTGATAAAACAGGGTTTGAGAAGATTGACTCCAGTTTCA
AAGACGTTCTACTGTGAGTAAAATGCCATCTGACAGCATAACATGTTACAGAGAAATCTTTTGTGAAAGGAAGAGTCAATCAAATGC
TGCAAACTTCATTGTTGTCTTATTTTAAGAAATTGCCACAGCCACCCCAACCTTCAGCGCCACCACCGTCATCAGTCAGCAGCCATC
AACATTGAGGCAAGGCCCCCCACCAGCAAAAAGATTACGATGTGCTAAAGGCTTAAATGATTTTCAGCATTTTTGGCTATAAAGTAT
TTTTAAATTAAGGTATGTACATTTTTTAGACATGATGGCATTGCACACTTACTAGATGCAGTATAGTATAAATATAACTTTTATGTG
TACTGAGAAAAAAATTGTGTGACTCGCTTTATTGCAATATTTGCCTTAGTACTGTGGTCTAGAATGGAACCTGCATATCTCCAAGAT
ATATCTGTAGTGTGATTATTTTGGAAAACCATTTAGTAGTTACCCAAGATAAGTTAAAACATGTCCACAAAAAAGACTTGTATGTGA
ATGTTCATAGCAACATTATTCACAGTGGCCAAAAACTGGAAACAACCCAAGAATCCATCACCTAGTGAATGGTTAAACAAATTGTGG
TATATCCATAAAATGGAATACTACTCAGCAACAAAAGGGAACAAATTTACAGATACATACAACAGCATGCATGAATTCCAGAAAAAT
TGTGCAGTCAAAGGAATAAAACTCAGAAGACCACATACAATGTTCATTCATATGAAGTTCTGGAACAGGCAAAACTAGTCTGTAGGT
ACAGAAAGCAAATCAGTGGTTTTCTGGAGTTTGACATGGGGGTACTAACAAGGGAACTTTTTGGGGTGGTGGGAATGCTGTATATTT
TGATTGTGGGGATGGTTACATGGTTGGATGCATTTGTCAAAACACACTTAATGGTAATGCAAAATGAATATATTTTATTTTATGTAA
ATTATACCTCAAAGTTGAATTTTTTTAAAAAGTTTCTTTTAAAAAGTAAAGACATTTGTGGTGCTTCTTGTAAATTTTACTGCTGAT
TATGACCTTGTTTCTTTAGATTTGACTTTCCAAGTTTGAAAAGACCAGTTTAAAAATGACTTTTGCTGGGCATGGTGGCATGTGCAC
GTAGTCTTACCTACTCAGGAGGCTGACGCAGGAGGGTCCCTTGAGCCCAGGAGTTGGAGGCTACAGTGAGCTATGATTATGCCACTG
CACTCCAGCTTGGGTGAAACTGTGAGACCCTGTCTCTAAAAACAAAAATAAAAAAAAATTAAAATGGCTTTTTAGATTATAATCTAT
ATGAAATTGGGGGTTATTTCGCACTTTTATATTATTTGTCCATTTTGCTTCTTTCTAAATAGCCTCTGGATTGTGTGTTATTTTCAG
AAAGCTCTTGGAGATTTTTTGAAGTGTATGTTTTAACAGGATTTTCCTGTTGCTCTTTGGCAAACTTTGTCCTTTTATTGTTTAAGG
AGTATAAATAACAACAGAACAGAATTACTTTCTGGTGCATTTTCCTCTTCTCCTGAGTCTTTTATGTTCAATAACTCTGAAGTTCCT
GTTATTCCTGCCTTTGAACGTAGAAAATGAGACTCTCTCTCAAATACCTGAAACTAAGGCACATAAAAAATTTAGGAGGGGCCAGGC
ACGGTGGCTCATGCCTGTAATCCCAGTACTTTGGGAGATTGAGGCTGAGGTGGATGGATCGCTTGAGCTTGGCAGTTCAAGACCAGC
CTGGGCAACATGGTGAAACCCCATCTCTACAAAAACAAAAATTAGCTGGGTATAGTGGCACGCACCTGTAGTGCCAGCTACTTGGGA
GGCTGAGGTGGGAGAATTGCTTGAGCCCGGGAGGTGGAGGTTGCAGTGAGCACAGACCGCGCCACTGCACTGCAGCCTGGGTGACAG
AAGTGAAAAGAAACACTGTCTCAAAAAAAAAACAGGAGGGAGGAGCCAAGATGGCCGAATAGGAACAGCTCCGGTCTACAGCTCCCA
GCGTGAGCGACGCAGAAGACGGGTGATTTCTGCATTTCCATCTGAGGTACCGGGTTCATCTCACTAGGGAGTGCCAGACAGTGGGCG
CAGGCCAGTGTGTGTGCGCACCGTGCGCGAGCCGAAGCAGGGCGAGGCATTGCCTCACCTGGGAAGCGCAAGGGGTCAGGGAGTTCC
CTTTCCGAGTCAAAGAAAGGGGTGACGGACGCACCTGGAAAATCGGGTCACTCCCACCCGAATATTGCGCTTTTCAGACTGGCTTAA
GAAATGGCGCACCACGAGACTATATCCCACACCTGGCTCAGAGGGTCCTACGCCCACGGAATCTCGCTGATTGCTAGCACAGCAGTC
TGAGATCAAACTGCAAGGCGGCAACGAGGCTGGGGGAGGGGCGCCCGCCATTGCCCAGGCTTGCTTAGGTAAACAAAGCAGCCAGTA
AGCTCGAACTGGGTGGAGCCCACCACAGCTCAAGGAGGCCTGCCTGCCTCTGTAGGCTCCACCTCTGGGGGCAGGGCACAGACAAAC
AAAAAGACAGCAGTAACCTCTGCAGACTTAAGTGTCCCTGTCTGACAGCTTTGAAGAGAGCAGTGGTTCTCCCAGCACGCAGCTGGA
GATCTGAGAACGGGCAGACTGCCTCCTCAAGTGGGTCCCTGACCCCTGACCCCCGAGCAGCCTAACTGGGAGGCACCCCCCAGCAGG
GGCACACTGACACCTCACACGGCAGGGTATTCCAACAGACCTGCAGCTGAGGGTCCTGTCTGTTAGAAGGAAAACTAACAACCAGAA
AGGACATCTACACCGAAAACCCATATGTACATCACCATCATCAAAGACCAAAAGTAGATAAAACCACAAAGATGGGGAAAAAACAGA
ACAGAAAAACTGGAAACTCTAAAACGCAGAGCGCCTCTCCTCCTCCAAAGGAACGCAGTTCCTCACCAGCAACAGAACAAAGCTGGA
TGGAGAATGATTTTGACGAGCTGAGAGAAGAAGCCTTCAGACGATCAAATTACTCTGAGCTACGGGAGGACATTCAAACCAAAGGCA
AAGAAGTTGAAAACTTTGAAAAAATTTAGAAGAATGTATAACTAGAATAACCAATACAGAGAAGTGCTTAAAGGAGCTGATGGAAGC
TGAAAACCAAGGCTCGAGAACTACGTGAAGAATGCAGAAGCCTCAGGAGCCGATGCGATCAACTGGAAGAAAGGGTATCAGCAATGG
AAGATGAAATGAATGAAATGAAGCGAGAAGGGAAGTTTAGAGAAAAAAGAATAAAAAGAAATGAGCAAAGCCTCCAAGAAATATGGG
ACTATGTGAAAAGACCAAATCTACGTCTGATTGGTGTACCTGAAAGTGATGTGGAGAATGGAACCAAGTTGGAAAACACTCTGCAGG
ATATTATCCAGGAGAACTTCCCCAATCTAGCAAGGCAGGCCAACGTTCAGATTCAGGAAATACAGAGAACGCCACAAAGATACTCCT
CGAGAAGAGCAACTCCAAGACACATAATTGTCAGATTCACCAAAGTTGAAATGAAGGAAAAAATGTTAAGGGCAGCCAGAGAGAAAG
GTCGGGTTACCCTCAAAGGAAAGCCCATCAGACTAACAGCGGATCTCTCGGCAGAAACCCTACAAGCCAGAAGAGAGTGGGGGCCAA
TATTCAACATTCTTAAAGAAAAGAATTTTCAACCCAGAATTTCATATCCGGCCAAACTAAGCTTCATAAGTGAAGGAGAAATAAAAT
ACTTTATAGACAAGCAAATGCTGAGAGATTTTGTCACCACCAGCCCTGCCCTAAAAGAGCTCCTGAAGGAAGCACTAAACATGGAAA
GGAACAACCGGTACCACCCGCTGCAAAATCATGCCAAAATGTAAAGACCATCGAGACTAGGAAGAAACTGCATCAACTAATGAGCAA
AATCACCACCTAACATCATAATGACAGGATCAAATTCACACATAACAATATTAACTTTAAATATAAATGGACTAAATTCTGCAATTA
AAAGACACAGACTGGCAAGTTGGATAAAGAGTCAAGACCCATCAGTGTGCTGTATTCAGGAAACCCATCTCACGTGCAGAGACACAC
ATAGGCTCAAAATAAAAGGATGGAGGAAGATCTACCAAGCCAATGGAAAACAAAAAAAGGCAGGGGTTGCAATCCTAGTCTCTGATA
AAACAGACTTTAAACCAACAAAGATCAAAAGAGACAAAGAAGGCCATTACATAATGGTAAAGGGATCAATTCAACAAGAGGAGCTAA
CTATCCTAAATATTTATGCACCCAATACAGGAGCACCCAGATTCATAAAGCAAGTCCTGAGTGACCTACAAAGAGACTTAGACTCCC
ACACATTAATAATGGGAGACTTTAACACCCCACTGTCAACATTAGACAGATCACCGAGACAGAAAGTCAACAAGGATACCCAGGAAT
TGAACTCAGCTCTGCACCAAGCAGACCTAATAGACATCTACAGAACTCTCCACCCCAAATCAACAGAATATACATTTTTTTCAGCAC
CACACCACACCTATTCCAAAATTGACCACATAGTTGGAAGTAAAGCTCTCCTCAGCAAATGTAAAAGAACAGAAATTATAACAAACT
ATCTCTCAGACCACAGTGCAATCAAACTAGAACTCAGGATTAAGAATCTCACTCAAAGCCGCTCAACTACATGGAAACTGAACAACC
TGCTCCTGAATGACTACTGGGTACATAACGAAATGAAGGCAGAAATAAAGATGTTCTTCGAAACCAACGAGAGCAAAGACACCACAT
ACCAGAATCTCTGGGACGCATTCAAAGCAGTGTGTAGAGGGAAATTTATAGCACTAAATGCCTACAAGAGAAAGCAGGAAAGATCCA
AAATTGACACCCTAACATCACAATTAAATGAACTAGAAAAGCAAGAGCAAACACATTCAAAAGCTAGCAGAAGGCAAGAAATAACTA
AAATCAGAGCAGAACTGAAGGAAATAGAGACACAAAAAACCCTTCAAAAAATCAATGAATCCAGGAGCTGGTTTTTTGAAAGGATCA
ACAAAATTGATAGACCGCTAGCAAGACTAATAAAAAAAGAGAGAAGAATCAAATAGACACAATAAAAAATGATAAAGGGGATATCAC
CACCGATCCCACAGAAATACAAACTACCATCAGAGAATACTACAAACACCTCTACGCAAATAAACTAGAAAATCTAGAAGAAATGGA
TACATTCCTTGACACATACACTCTCCCAAGACAAAACCAGGAAGAAGTTGAATCTCTGAATAGACCAATAACAGGCTCTGAAATTGT
GGCAATAATCAATAGTTTACCAACCAAAAAGAGTCCAGGACCAGATGGATTCACAGCCGAATTCTACCAGAGGTACAAGGAGGAACT
GGTACCATTCCTTCTGAAACTATTCCAATCAATAGAAAAAGAGGGAATCCTCCCTAACTCATTTTATGAGGCCAGCATCATTCTGAT
ACCAAAGCCGGGCAGAGACACAACCAAAAAAGAGAATTTTAGACCAATATCCTTGATGAACATTGATGCAAAAATCCTCAATAAAAT
ACTGGCAAACCAAATCCAGCAGCACATCAAAAAGCTTATCCACCATGATCAAGTGGGCTTCATCCCTGGGATGCAAGGCTGGTTCAA
TATACGCAAATCAATAAATGTAATCCAGCATATAAACAGAGCCAAAGACAAAAACCACATGATTATCTCAATAGATGCAGAAAAAGC
CTTTGACAAAATTCAACAACCCTTCATGCTAAAAACTCTCAATAAATTAGGTATTGATGGGACGTATTTCAAAATATAAGAGCTATC
TATGACAAACCCACAGCCAATATCATACTGAATGGGCAAAAACTGGAAGCATTCCCTTTGAAAACTGGCACAAGACGGGGATGCCCT
CTCTCACGGCTCCTATTCAACATAGTGTTGGAAGTTCTGGCCAGGGCAATCAGGCAGGAGAAGGAAATAAAGGGTATTCAATTAGGA
AAAGAGGAAGTCAAATTGTCCCTGTTTGCAGACGACATGATTGTTTATCTAGAAAACCCCATCGTCTCAGCCCAAAATCTCCTTAAG
CTGATAAGCAACTTCAGCAAAGTCTCAGGATACAAAATCAATGTACAAAAATCACAAGCATTCTTATACACCAACAACAGACAAACA
GAGAGCCAAATCATAGGTGAACTCCCATTCACAATTACTTCAAAGAGAATAAAATACCTAGGAATCCAACTTACAAGGGATGTGAAG
GACCTCTTCAAGGAGAACTACAAACCACTGCTCAAGGAAATAAAAGAGGACACAAACAAATGGAAGAACATTCCATGCTCATGGGTA
GGAAGAATCAATATTGTGAAAATGGCCATACTGCCCAAGGTAATTTACAGATTCAATGCCATCCCCATCAAGCTACCAATGACTTTC
TTCACAGAATTGTAAAAAACTACTTTCAAGTTCATATGGAATCAAAAAAGAGCCCACATTGCCAAGTCAATCCTAAGCCAAAAGAAC
AAAGCTGGAGGCATCACACTACCTGACTTCAAACTATGCTACAAGGCTACAGTAACCAAAACAGCATGGTACTGGTACCAAAACAGA
GATATAGATCAATGGAACAGAACAGAGCCCTCAGAAATAATGCCGCATATCTACAACTATCTGATCTTTGACAAACCTGAGAAAAAC
AAGCAATGGGGAAAGGATTCCCTATTTAATAAATGGTGCTGGGAAAACTGGCTAGCCATATGTAGAAAGCTGAAACTGGATCCCTTC
CTTACACCTTATACAAAAATCAATTCAAGATGGATTAAAGATTTAAACGTTAAACCTAAAACCATAAAAACCCTAGAAGAAAACCTA
GCCATTACCATTCAGGACATAGGCGTGGGCAAGGACTTCATGTCCAAAACACCAAAAGCAATGGCAACAAAAGACAAAATTGACAAA
TCGGATCTAATTAAACTAAAGAGCTTCTGCACAGCAAAAGAAACTACCATCAGAGTGAACAGGCAACCTACAACATGGGAGAAAATT
TTCGCAACCTACTCATCTGACAAAGGGCTAATATCCAGAATCTACAATGAACTCAAACAAATTTACAAGAAAAAAACAAACAACCCC
ATCAAAAAGTGGGCGAAGGACATGAACAGACACTTCTCAAAAGAAGACATTTATGCAGCCAAAAAACACATGAAAAAATGCTCATCA
TCACTGGCCATCAGAGAAATGCAAATCAAAACCACTATGAGATATCATCTCACACCAGTTAGAATGGCAATCATTAAAAAGTCAGGA
AACAACAGGTGCTGGAGAGGATGTGGAGAAATAGGAACACTTTTACACTGTTGGTGGGACTGTAAACTAGTTCAACCATTCTGGAAG
TCAGTGTGGCGATTCCTCAGGGATCTAGAACTAGAAATACCATTTGACCCAGCCATCCCATTACTGGGTATATACCCAAATGAGTAT
AAATCATGCTGCTATAAAGACACATGCACACGTATGTTTATTGCGGCACTATTCACAATAGCAAAGTCTTGGAACCAACCCAGATGT
CCAACAATGATAGACTGGATTAAGAAAATGTGGCACATATACACCATGGAATACTGTGCAGCCATAAAAAATGATGAGTTCATATCC
TTTGTAGGGACATGGATGAAATTGGAAACCATCATTCTCAGTAAACTATCGCAAGAACAAAAAAACCAAACACCGCATATTCTCACT
CATAGGTGGGAATTGAACAATGAGATCACATGGACACAGGAAGGGGAATATCACACTCTGGGGACTGTGGTGGGGTCGGGGGAGGGG
GGAGGGATAGCATTGGGAGATATACCTAATGCTAGACGACACATTAGTGGGTGCAGCGCACCAGCATGGCACATGTATACATATGTA
ACTAACCTGCACAATGTGCACATGTACCCTAAAACTTAGAGTATAATTTTTTTTTTTTTTTTTTTCAAAAAACAGAGATCCCTTAGT
TAGGTAAACCAGGCCCTTTCTGATTGGTTGCTGTGAATCTCCTGGTTTTTTGAATACTGGCTCATTTTGAAGTTCAGTTTCATTATG
TGGCACCGAACAGGAGCGATTCCATACTGGTTCTGTCTGGTCTTTTGGGGCCTAGAGCACAGCCTCAGCCCTAAACAATGGCTTCTC
TTCCACTTTGTTTAACAGTTCTCTGAACTCTTTCAAATTAACGTTTCCGTATGGCATGAGATAAAGGTAATGGTTCATTTGTTCTCC
ATGTGGCTATCTAGTTGTTATAACACTATTTATTGAAAAGTCTATCTTTTCTTTATTGAATTACTATCATATCTTGTTAAAAATCGA
GGATATGTCTGTAGGTCAATTTCTGGATTCTCCATTCTGTTCCATTGATCTCTTGGCCATATACCACACTGTCTTGATTACTGTAGC
ATTATAATAAATCTTTAAATAAGGGAGTATAAGTCCTTCAACATTGTTCTTTTTAAAAATTCTTTTGGCTATTTTTGGTTCTTACCA
TTTNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNTTTTT
TGTACTAAGTCATTGAAATCTGGTGTGTTTTTACACTTACAGCACACCTTAGTCACCTTAGTCTGAATTAAGATTCAGACTAGCCAC
ATATCAAGTGTTTAGTAGCCTCATGTGGCTCATGGATACCATACTGAACAAAATAACTATAGATCAATTTGGAGATAATTGTCAAAT
AAATAGTATTTAGTATTCTTAACAAAAAATGAATTTAAAAGTGTATTATTAAGTGTCTTATTTATCTAAACAGGTATTAGTGTACAG
TTCTTGCATGTACTTTGTTAAATTTATTCCTAAGTACTTCAGGTTTTTGCGTGCTGTTATTAATGGTATTGTTTTTCATGTTTTAGT
TTTTAATTGTTCATTGCAACTTATATAAATAATTGAATTTTGCGTATTGGCCTGGTATATTCTGCAACTTATTGAAAGTGTTTACTA
GCTCTGGTATGTTATTTTGTTGACACTTTAGAACTTCCTACCTAAAAAAATATGTCTGTAAATAAAAGCAGTTTTCACTTATTAAAA
AAAAAAAAACAAAAAACAAAAAAAAGGAAAAGCATGGCAGGGCAAAGTTGCTCATTTCTATTGGGCTATGACTGGGAACCAAGAGTT
TAGGGAAGTTGGTTTCCTACTTGATGTACACACCAGTCTTTGAGGTTTACTGATTACAGGTACCCAAAGTGTGTGTTCCTGCTTGCC
TTCTCAGGTTCCTGTGTGGATGGATGGTTATCTTAATGCTGGTAATTAGGCTAAAGGAGCCATTTCACCCTTGTGGGGTCTTAGTGA
GCTGATAATAGACTAGTCTTGGAGTTCAGTTGACCTGTGTTCAGAACATGACACTGATCAGCACTGTGACCTCGGACAAGTTAATTC
ATCTCTCTCTCTCTCTTTTTTTTTTTTTTTTGAGACAGTCTCGCTCTGTCGCCCAGGCTGCAGTGCAGTGATGCAACCTCGGCTCAC
TGCAACCTCTGCCTCCCGGATTCAAGTGATTCTCCTGCCTCAGCCTCCTGAGTAGCTGGGACTACAGGCACATGCCACCACACCTGG
CTAATTTTTTGTATTTTTAGTAGAGACGGGGTTTCAGCGTGTTAGCCAGGATGGTCTCGATCTCCTGACCTCGTGATCTGCCCACCT
CGGCCTCCCAAAGTGCTGGGATTACAGGAGTGAGCAACCACGCCTGGCCAATTCATCTCTTGAGCCTTAGTGTCCTCACCTGTAAGA
TGGGAATATCCACTTTACAGAGTTTTCTGGAAAATTAGATGACATGAAATAAGTGAGAGTCATAGGTGTTGTGCCTGGCACATCATA
GGCACTTGGTAAACCTGAGTTCATTTCCTTCCTTTATTCGGCAGCTTCCTGGCTCAGGTCCCTATTTCACATAAATTGTATGCCTAG
CAGCACCCTATTCATTTTATAAATTGAATATGAGAATCAGATGATAAGAGTTTGGGACCATCTTTAGCAGATGGTTTTTGTTAAAGG
TACCTTTTGAAGTGGGGGTAGTGCCGTTTCATTTCATGGTCTGTGTTGTGTACCTTTGCAGCATGTCTTTGTGATATGCAGTGATGT
CACTGGAATTTCGTACAGTAGGATGGGATACTGAAAGTAAATCTGATCATTGGTAGCACCTTTCCTGTAATTACAGCACAGGAGAGA
GGTAGTGTGACTATGTAAATAGAGCTAGAAGTGCTATTGAAATAACTCAGCAGATTCTTTAAAGAGAAAAAGGGGAGAAAAACCCCA
CCACCTTCCACAGAGACATTCCCATGCTAGGCCTGCACTGACACTGCAACACCAGATAGACGATTCAGTTTATTTTGCTCCAACATT
TTACGCGTTGGTCAGAGAAAATGGTTTTCATTAACACAAGGTAATGAAAAATTTTGTGCCATGGTATCATGTTTGTAAAAAGCTTAA
TTACATTGTTGCTGTTTCTGTTTTGAACCTAATTCTGGTAGTCTGCGCGTAGCTGGCGTGCCAGTTCCGGGTTGTGGGTCTAGGCAG
TTGGAGAAAAGAAGCAATTATCCAGTTAATGTAAAGAATCCTGCTAAGGAGGAAAGAAAGGTGCCCCTCATGCCCTCATCAGTCACT
CCAATTGGTGTTTATAGGGAATTAAGTTCTCTGGTAATTGCCCTCCTTCCCTTTCCTTTGTCTGCTAGGGCCATCATCACAGGCCTT
AAGTGAACAACTCTGGGAGTATTTCCGGCCTCTTCTCTAAGTCAGGCCAGTAATGCAGCTGTTAAGCACTGACATCTGGGTCTCAGT
CATGCTTCCAGACCAGGATTGTTCTTAGTTCCAGGAGATTAATTGACCCATAAGAAATATTATAATGCAAATATATGTTTGCGCCAA
TTTGCTGACCAGTGCTTGCTATCCTGGAATTCACAGTGTGGCAGAATACAACTGTCTGGCATTAAGGATAACCTAGGTCTTGTTTCC
ACTTGTTTCCAGCATGTACTAATCTAGGGTGAACTTTCTTCACACGTAGAGAGCATCAAAGAGGAAGGTGTTGAAGCAGGAGCCCTG
GATTAGGGATCAGGAGTGTGGAGTCTGGTTCCAACACTGTCACATTTCTGCTGTGTGTCCCTTTCCAGGCCCTTAACCTGTGCTGGG
CCTCAGTTTTCTCATGTATAGAATGCAGTAATCATAGCTTAATTTCTAGCCAGCTTTCTAGGGTTGTTAATCTATCCTTTTATCAAA
AGGATAAAATGATATGTTATAAATGAAGACACCTTAAAAAACAAAGCACTCTGCAAACGTAAGATGTTATTAAATGTAAACTCAATG
ACATTATTTCAACATTCACATTAGAGAAACAACATTTGAAGTCTTCTAGGCCACACACAAGTTATTGTAGAGCTTAAATTTCTCTTT
GGTGAAGTTCTTTTAGGACAGATAAATATGCCTCAGGGCCAGCTTATCTCTTTGAACATCGTAACTGGATATTCTTTGGCTTCCTTT
TTCCATTATTTAATCTTACTAAATTTCTCTTTTATTTTGAAGCATTGGGAATAATGAGAAAACTCGAAGATTTCAACAATAATGAGT
GGAATAATATGAAGGACCTATGGGAAGAGAGAATAAATTATATTAAATATATTCAAAGCAAAAGATGAGAAAAGACATTCTTTAAAA
AAATAGTGATAATGTTAAAATATCTTTTTTAGAAGTAATAGACTTGGGAACAGAAAAGGGATGTTGTTAACTTTTTTTTTTTTTGAA
CCGAAGTTAACACCCAGCACTGTCCTTTCTTTACTTAGCGAAACATACGGTGGAAACAGATTTCTGTGATCTTCTGCGGTCTCTGTT
ACTGGCCACCCCATTCATGTGACATCGGCACACTTGGCTCATTTAAAAGGCTTCTCAGTGATGTCTTGAAAGCCTAATCCTTTTTCA
TTTTATCTCTTTCATCCTGCAGGCCATGATGAAGTTAATTCGTTTTAACTTTCCGAGACACAACCACTTTTACAAAACAGGAAAATG
AAAGGACTCTGAAAAGTGTGACACAGAATATCTAGTTTGCAGAATAGACTGTGCAGGGCGGGAGGATGGCCGACGACGGGTTTCATT
TAGGAACTCAAAGTGAAAGGACAGGGCAAGAAGTGTAGTGAACACCTTCAGGGAAATGCCGAAGAGACCAACTGTTATTTGAAAACT
TGGATAGGTTTTGGGGCCCCAGATCCAAGCACCTTGTTTTGTTGTTGTTGTTGTTGTTGCTGTTTTTGAGACGGAGTCTTGTTCTGT
CACCAGGCTGGAGTGCAGTGGCATGATCTTGGCTCACTGCTGCCTTCGCCTCCTCAGTTCAAGCGATTCCCCTGCCCCAGCCTCCTG
AGAAGCTGGGACTACAGGCACGCACCACCATGCCCAGCTAATTTTTTTGTATTTTAGTAGAGACAGGGTTTCACCATATTGGTCAGG
ATGGTCTCAATCTCCTGACCTCGTGATCTGCCCGCCTCAGCCTCCTAAAGTGCTCGGATTACAGGCATGAGCCACTGCGCCCGGCCA
GCACCTTGGTGTTTATGGAGATGGCGATGTGCCTTGCTTCACTGGGGTATGGATGGGCGGAGTTTCTGTTCCAGACTTGCTTGTCTT
CCCCAACCCTGCCTACCTTCTTTAGAGGAGGAAATATATGTTCAGTCTTCATGGAGCTAAGAAAAAGCCAGACAGCCGAGGAAGAAG
CTAATAATTAGGCTTCTTTGGAAGGTATGAGACAAATTGGCAGCATGGTCACCCGGGAAAACACAGTGGGGCTTTGTCTGCAACTAA
GAACTCTAGAAATCATGGTGTCTGGATACCACGGGGTTAAAGCTGTGGAAGCCTCAGCCTGCGTGTGGAAGCTGGGCTTTCTAATTG
GATCACTGGCACACACAGTGCTGTTGCTTCTTCTCTGCCCATGTCCCCCAGAGCATCTATCTGCTTTTGTTACTGCATTGTTATGAA
TGCCCTTCTTTGGCGCCCTCCACTTTTCCTGAAGGCAGGCTGGGAAGCAGCCCTTGGCATGTGTGATGGCTGCGCTGCATGCTGGCC
ATCGAAGGATGTCCATCCAAGGAAAAGCTGCTCTCCCTCAGCCCTCACCCTTGGCTTTTGCCCCACCCTGCCTCCCAACTGCCGGTG
CATTCAATTCCCTGCCTTTGTCCAAGGGGCAGGACAGGGCACGACCACTTTGGCTCCCCTTTTCCCTTTGGAAGCCAAAGTCACTCC
TCCAGCTTCAGAACTGTGAGTTTCTTGTGCATGCCGCTGGCTTGGTGGGAATATGAGAATGCGAACTCTAACTCTCACTGTGTGAAG
AGTTGGCAAGGGTGCATGTGCACGCTATTGTGTGTTATCTGTAAGAGAACTGCAAAAACAAGTGTCTAGAATTAGATTCAGTGCTTC
AGGACCCCCTCTCCTTTCTCCATTTAGATTCCTCAACAGAAGCTGCTTTTCGCTTGATTAACTGTAACAAACTCTCTTTGGAGTTTT
GTATTTTGGAAACTGCATTGCCAAAGGAAAATCATTATGTCTGCTGTAAAAGCCCCAAATAAATTCCTCCTAGGGTAAATGGAGGAA
AAAGAATAGAAGGGTTTAATCTATCATCTTGCCTATTAGTGAAATACTTAGTGACCTAGTTCATTTACATATTAAATGTGCTAGACC
TATTTGTGCCTTCTTTTTTTTTAACTTGTTACCCCGGAAACCATGCCCTGGCTCAGAAATTACACAAGGCCAACAGCAAATCTGCAG
TCTGGTTTCTTCACACAGACAAGTCAGTTAATGGGGAAAGGTTAGATTGTCCCAAAATGTCTTCAGTCGTTAGTAGAAAAAGGAGAG
GAAGGAGAAAAAGTGAGAAACATGTATTATTTTGGTAACATTTTCAAAATACTACTTTTAAACTAATTGGAAGGGATTTTACCTAGA
CTTAAGAAGAAAATAATAGTGTGGACTCTTAAAACTTCAGTCTGCTTTTAAAGAACTTTCCTGGTACATTTGGCAGCACTGGCATGA
AAAGGATCAGCTATCAATTGACATTGAATTCCAGACCTACATTTAAGGAATCTTGACTCTCTCATTGCTTTGTTGTAATAGCACTGG
TTCTTCCAGATTGTTTGGTGGTGTCTTATGTTCTCAGGCCAACTTTTCAAGAGTCTGAGAAGCAGAATTTAAAAATACGATTTAGTT
AGCAGCTAGAGAGCAAAATTTCAGCCCATCTTCCCACCTGAATATGTAGCCTGCAAGTTCTGGGAGAACAAGGAACTTTACTGATCT
TCTACAGTGTCTCATTTCACAGTGGAGGAGATTGGCACCCAGAGAGGTGAAATAAATTGCCAAGGGCATCCATCTGGTTTGGGGCAG
TCTAGACTAGAAAACAGGCTTCTTAACACATCTTGCAGCATCATGGATGGAACTGGAGGCTATTATCCTAAGTGAAATAACTCAGAA
ACAGAAAGTAAAATACTACATGTTCCCACTTACAAGTGGAAGCTAAACAATGGATACACATGGACATAGAGAGTGGAATAATAGGCT
GGGCGCAGTGGCTCACGCCTTTAATCTCAGCACTTTGGGAGGCTGAGGCAGGTGGATCACTTGAGGTCAGGAGTTTGAGACCAGCCT
TGCCAACATGCTGAAAACCTATCTCTACTGAAAATACAAAAAATGGCCAGGTGTGGCGGTGCATGCCTGTAATCCTAGCTACTTGGG
AGGCTGAGGCAGGAGAATTGTTTGAACCCCGGAGGCAGAGGTCGCAGTAAGCCAAGATTGCGCCACTGCACTCCAGTCTGTGTGACA
GAGCAAGACTGCCTCAAAAAAAAAAGAGAGAGAGTGGAATAATAGACATTGGAGACTACCAAAGGTGGGAGGCTGAACGTTGAAGTA
TTGCTTGTTTGTTACAGTGTTCACCATTCGGGTAATGGGTACACTACAAGCCTAGAGTTTGTTGCTATGCAATATATGCACACCAGA
AAATATGCACTTGTACCCCCTAAATGTATAAGAATTTTCAAAAAACTTTTTAAAAATTAGGTTTTTTGACTGCAGATCTAGTGCTCT
TTCAACATTAGTCCTGACACAGATAAGTCACTTGCTTTGAGTTCTGTCAGAGGTAAAGCTGGGTGAAGAAATTGTGAGAAGAATGCT
ATTCTCTTTAAGCCAGCCACCAGCCAGCTTACTTGGGAAGCGGTGGAAGGAGGACACAGTGTATCCTCAAATGTTAAATCCCTTCTT
AGTGGTATTTGTTGTGGATAGAGGAATTAGATTGAATGTGGAACTTTGAGCACTCTGAGGATAAATTGTGCCTTCTCATTCAAGAAC
ACATTCAATTCTCTTGGTCACTCCATCTTTTCTGAAGTTCCTTTTACCATGCTTTGCAGTGGCGATAGGCCTATTGCATGAAGAGAT
AAAATGTCTTTTAGGCTTTTTCCTCTTAAAAACTTGGGATAAGAACAAGTTAGCCTGAGGCAATCACAGAGTCTTTATAAAGAGACT
TTTAGCAAGGAAATGATTTTTCAAATACAGTACAGGATTTTGGGCATGACTTTGAACTGGAAAGTCTACATATATAAGTAGAAGATG
GTGTGTAATATCTTTGCAAATACTTTTGTGTATTCATTCCTCATTTGTAAATGTTTTCTTACCTGCTGACTCAAGCACTGACAGTGT
ATTTGTTTGCTCATTCAACTATGGACTGTGAACATGCTTGGGAAACAGAGTTGAGTATTGTACGCAATCTCTGACCTCAGTAATACT
TAGGCATATATGCTTATTCAGTAACTGGACATTTTAAAATAAAAAGAAATGAGTATTTTCACTGCTTGCATATAATGGGAATGATTG
AATAACTTTCCCATAAGATGGCATTAGTTCAAGTGTCCTTATTTTAGTAAGAGTATTTCAGGCCAGGTGTGGTGGCACACACCTGTA
GTCCCAGCTACTCAGGAGGCAGAGGCAGGAGGATCCTTTGAGCCCAGGAGCTAGACTCCAGCCTGGGTAACAAAGAAAGACCGCATC
TCTTTGAAAAAATAATTTGGAAGCTCTCCGTAAAAATCTCACCTCTTATTTTCCTCAAAACAAGCTCTGGTGCATTAGGGTATATAG
TAGGGTCCCCTGGCCTTTGGAATGGGGGGTTATTTGAGACTGGAAGGTGGGAGTGGGAGAGGGAGGGGGAGCCAGGGACAAGCATGG
CAAAGTTCCAGATTCTCTGTATTTGTGTGAAAAATCACAAAACCCTACATTGACTGTGTAGTGGATGTCAGATGGGAAGAGGGAGTG
GGGAAGCAATGAATAATAGCAATCACAGGTGCAGGAGCCACGTCTCCTCCTGGGCACTTTACATGCGTTCTCTCACTTATTCTTCTT
TCTCACCCTGTGAGGCTTGGTCCAGTTGCTATTCCCAAATCACCCATGAGGAACCTGATGCTGTGAAGATCAGCAGCATGGCCAAGG
CCGTGTAGTGAAGTGATGAGCAGGAGTTAGTCCCGGTCTTTCTGTTGTCTGAGCTTACCAGCTCTTGCCTTCACACAGTGCTCTCTC
CTCTCTTATCAAGTTGAACGTGGGTCAGGACCATGAAGTTAATTTTCTTCCTCCTTTTAATCATCAAAATGGAATTTCTGTTTTGCC
TGTTAGAGATTATTGATATAAGAAAGGCTGGGCGTTAAATGCTGAGAACACATGGACACATAGAGGGGAACAATCACACCCTGGGGC
CTGGTGGAGGGTGGAGGGTGGGAGGAGGGAGGGGATTAAGAAACAACGAATGGGTACTAGGCTGAATACCTGAGTGATTAAACAGTC
TGTACAACAGACCTCCATGACACAAGTTTACCTGTGTAACAAACCTGCATTTTAAAAGTTAAAAAAATGAAAGGCTTTATATCAATA
AATAAGTGGGCCTAAAAAAGGGAGACAAAATGAAGGAAAATTATTCTGGATTCTTAAGAATAAAAGTGAAAATATCGGACTAAACTG
ATAAAATGAGGTAGAAAATAGAGGAATCACATACACAGTTTTGACTTCCAACCCACTTTGCCTTAAGTTATTTCAGTTGACAAGTCG
AAGTTGGGTTGAGGGGATGAGGGTAGGAGGTGAGTGATGTATGGATAAATACAACGTTCTATTTTCTAATGGTAAGTATATCTGCCT
TCCCTCCCTCCAGTGCTTTTATACGGTACCAAGGAATATGTTTTGCTTTTTGTCTTGACAAGGGAATTCACAACTCATAACAGCTAT
TTGACATCTTTTGTGGGTACCAAGCTCACTTTTCTTCTTTCCAGTGATAAGTGCCTTAATAGAACAAAGTCAACATAATCCTTGCCT
CCAGGGAGCTTGCAGTTTAATGAGGGGGGCACATGTTTACAATATAGTGGGGTCAGTGCTGGGGAGGCATTGAGGAAGAACTTAGTG
TATGAATCCATCTCCGAACTTCAGTGATGTGGAAAATAAGCTGTTAGAAAGATATCTTGGACTTCATTTTTCAAGCAGCTTGAACTC
CTCAAACAATTGTTCTGCAGACTTATTTGCCACGAGATTTGTGACTTGAAGTCTAGTGTTTAGATCACAGTGAGCTTTTTGGAGATG
AGATTTGTGCTCACATCTGTGGCAAGTCAACGTGTGGCAAATGCCTGTTTTGACTGTCAAAACGGAAAGCAAATCTCTTTGCGTGAT
TTTCTATGTCATTTTCTTTATTAGTGCACATGTCATGGGCGTTTCTTTTCCTTGGCGTTTTGTGTGAACTTAATCCTAGACAGAACC
TCAGTTTGAGTGGTTAAGGCTCAGCATCCTGGTCAGCGTCTGAGCTGCTGTCCTCTGGGTCCCTGTTGTGCCGTCAGCTCCAGTGGG
GATGGCACCAGGTTCAGGAGGCTGAAGAAGTGACCCAGAGCCAGCAAACGCAACATGCGGTTGCGTCGGGGGCTTACATACAGGGGA
GAGGGCCCAGCGGCTATGCGCTGGACAAGAGAACCGCAATCACTTGCAAAAGCTATGCAGTTTATATTGCATTTTCACTTAGTACTC
TGTTTAACGATTCCACCTGGCAACCTTCATTCATGCCAAAACTCGGGGCCTCCATTTCCCTACAGCCCATGTCCCACAAGAGATGGG
CCGGGAGCTCAGATGTTCCTCATAGACAAGGAACAGATCTCTGGGGTGGCCACTTCCAGATCCCTAGCTCAGAACTCTGAACACATT
CGGGGCATCTGCCATACAGAATCATTCTTAAGAGTACGCTTAAGTTACTGCTATCAGGTGCATTTACTGTATACCCCTAAAGTGACT
TCAACTTCTAGTCAGTCTAATGAAATTTGATTTCATGCAGAGGGTCTCTTAGACAAAAATTACATCTATAAGCAAATTACTGGCTGC
GCAGGTTACTTTGAAATATTTCTGGTGTTTTTTTAAAAAAATCAGTTAAGCTGATCAGAGACAAAGCGAAGAAATTACCCTTTTCTT
TTTAATGTGCAGCAAAATGTGTACTAATGGTGCAATCACAGAATATTAGGTTGTTAATACAATCAATAAAAAGTTAATCCGTTTTGG
ATTTCATTGTATTATGTTTTTCTTGTTTGTGGAATAACGTAATTGTGACAGAGTAATTAATACCCTGCAGAGGGATTAGCCGTCAAG
ATTGAGATAAAAGAACAAGGAGACAAGAGCAGAGTTGCCTAGAAGACAGGGGCGTTTAGAAAGTGCTGAGGCATAGAGTTTCCTTAA
GGTGATTTTTTACCTTTCCTGACTCTAATTATACATTTTTGCATCTAGTAGTGATACAGCTGGGTTGGTAAAACATATTATTAATAG
AAGTGAGGGAGTTCTGAGGTGGATCAGATTTTTCCCTTTAAGCTTGGTTTCCAGCCAAGCCACCGGTCCTCAATTGGTATTTTGCTG
TGTGTTCAGAATGGCTTTTTCACTTCTGTAATTTACTTATTTCTTATTATTTGTTATTCAAAGATCTTTTGGTCATTTATTCTGCAC
AAATCCTAGAAAATGAAAGCCAAATAAGGAGGGATTCCCCCAAATGACTACTAGTTTTTATATCAGTGCATGGTGGGCCTCTCAGTG
CCAACAGATTGTTCCCGTTAATTTTTTTTTCCCCTTTATATTGACTCTGGGGTTTCTAATGGTCAGCCTGTGCTTTCAGATTGGAGT
TATTAACTTTGACCAAAACAGGAGTGGTTTGAGAAGAGGCAGTAGAGAGGAGAAAGTGGAGATCTAGCCAGAATCTTTTGTTTTCCA
CAGAAATTAGAGAGGGAAAAGACTATAGGAATCAGACTCTGTCAGCCTTGTCCTTTGTAGCCCCTACCCAGGCAGCAAAGAGGAAAC
TGTCTTCTTCCTGTACGGGTCTCCAATTGGCTGACATTATAAATAGTCTCAGTGGCAGGTTTCTCATTTTTGAATAAAAAAACACGC
TTTCATGCTAAATTATTAAACATTCTGTGGTTTGGCTTTAAAATCAAATAAATAACTCTCTTTATGAATGCATTAGACACCCTCCCT
AGTTTCCTTTTAATTGTTTGTTCTCCCCTGTTCCTCATCTTGGACTTCAAGGCCATCATCTTGGTATTCTGGTGTCTGCTCTTCTCT
TGCTGACTTCTCTCTGGGAAACCTTTTCCAGGTCGGCTTGTTCCACTATCACCCACCTCCCTGTGAAGGAAGATTGAAGCTGGTGTA
GATGGGGCTTCCTGCCTGCCCTGGAAGTCTGAGCCAAGAGTAATTTTACCCTCATCTTTCCACAGTGTGTATAAGAGTAAAAAAGGT
CAGCTGGACACTTGCCTCGCTGGGATAGAGCAGTCAGAGTCAGATGATGTTTCTATGAACTGTTTTGTTTGGGGTCAGATGGACTCA
ACTGTGCAGTCCACATCATCTGCCCCCATCTGCACTGACCTGATATGGTGGGTGGCCACATCCTAAAATAGTTTCTCAGAGACTTTG
TGTGTGAAACATACATATAAATGTGTTCATGTTTATAATGTTGGTGTGCAAGAGTTTATTTTGCTTAAAGAAGATAATGGAAATTTT
GGTTTCAACTAACATATCTATTTTCTCTCCACTAATGAGTATGGAAGGTTTTCTCCTAGATGAAAACATGGAGAGAATCACTAATAA
GAAGAGAAAGCTGTTGAGTACTGTTAAAATATCACAAAACAGTTACTATTATAGTCATATATATGATACGTTCATTCTTTTAGAGGA
TAATTTAAACATTATTTTGGGTATATAGGGTTTTAAAAAATCTATTCAGCATCTAATATGAATGTGTGAAAAATTTCACATAAATTT
TCATATAGTAATTGGGACCAAATTATTTGCTGTCTTTTGTCTCTTTCACATTGTTTATGAAGATCTGGCTTGAATAAAGGAGTCAGG
AGCTTTCGCCTTCCATAGCTAAGCCCAACTGTCCTAATGCTGCATAAGACAGCTGGGATTAGAGAAACTCTTGAAAAGCAGGATTGG
TAATGGGAAGTGATAGCTGTGTGTGAAAGCCTTAATGCTGAAGTGCATTCTTGTAACGCAATTTTCTTGTCTGCCTTGTTATCTTAC
AATTTTAGTGGGAAAAATAATAACAATGTTATTTCAAAGAAATGGTTAAAAAATAGTCAAACTTAATAGAAAGCAGAGCCCTGTCCT
TTGGTTAAACACTCATTTACTCAGCTCTCTCTCTCTTTTTAAAAAACAGAACCATGAATCCTTCCTGTACAGTTGCATGGGTTGATA
TTAACTCTCTCTCAGCTTATTCACTGTCCACCTAAAAGAATAGTACAGGGCTCCAGAAATGTACCTTGCTCTGGTTTATAGGTAAAA
TGCTGCACTTGGGCATAGGTTTGTGGAGGGTGAGTATTGTTTCTCTTCCTGTGATGAAGATTGATGGCATTCCTGATAAAGGAAGAA
GAGCGTGTTTGAGGATGCTGGAAGATGCTGAAGTGAAGCTGAGAACGTGACGGCCAGCCTGGGCTCTTGGGGGTGGAGGATGGTTGT
GGATGCTGTGTTGCAGTTGACTATGCCCTTTCTGGAGGTGTACGGGTAGCACCTGTTCTGCTCTAACAGCTTTGTGGGGAGGACAGC
CACCCACCAGAGCCCCCCATCCAGCACGTCTTCCTATTTCCTTGGTATTAGTAACTACATGGGGGATATTTATCTATGTTTTAATGC
ACACAGGCTAGTGCAGTCAAGGGTGTGGGGCATACAAAGGTAAAAAATAGATCATTGTTACCCTATAGAAGCTTTCAGTTGGCATGT
GTAGTGGGAGGTGTGCTTTAGGGAGGTGTCATATGTAACTTAGAGGAAAGAATTCCCAAGGTGGCTGAGAGGACTGGGGAGGTGGGA
GAGTCTGCTTTGGATCTGGAGGAAAAGTGGCACTGTCTACCTAAGAGAATAGTGCCAAAGACAATTTGCACAGAGTTTGGCATATAA
TAAGTACTAATAGGCTAGGCACAGGTGGCTCATGCCTGTAATCCCAGCACTTTGGGAGGCCGAGGTGGGCAGATCACTCGAGGTCAG
GAGTTTGAGAACAGCCTGGCCAATATGGCAAAACCCCATCTTTACTACAAATACAAAAATTAGCTGGGCGTGGTGGTGTGTGCCTGT
AATCCCAGGGGTAGCTGAGACAGCAGAATCTCTTGAACCCAGGAGACGGAGGTTGCAGTGAGCCGAGATTGTGCCACTGCACTCCAG
CCTGGGCGACAGAGCAAGACTCTGTCTCAAAAAAATAAAATAAATACTAATAAATACTTAATGAAAGAAGGAGAAAAGTCTAGGGAA
GTTATTATTTGGGGAAGAGCTAAGAGGAATTCATCTGTGAATAGACAAACTGAATATCATTCCACGATTACTTTCACTTTAAATGTA
AGTATTTAAGGGTACCGTGAATGAATTCCTTCACAGATGTGTTGGCTCTGCAGGGATAAAGGTGATGGGGTTTCCAGTTCATGTTGC
AAGTTCATGTCTCAAGGGGAGGTTCTGTGAGACTTGGGCTGTGTCCTGACCACACAGCTTTGCATAATTGATTAATTCTGCATAACT
GCTTGCATTCCTTTCCTAAAATAACTTTCACTTTCTAATTAGCTCTCCAGAATTAATAAGTGCCAAAAAATAGGATGTGTGTTGAAT
CTTGTGATCTAATGTGATGCTGCCAACTCACACAGATTTAAGGTATAATTAATAATGCTGAATCTCTGAGGGAATTCTTAATTCCTG
AATTGGTAGACAGATCTCTAAAAAGTAGCTAATACTTATTGTGTTTACCATGTGCCGGGCACTGTGCTGAGGACTTTACCCAGTTTA
CCTCCTTGATCTGTTTTCATTATCACAAACACCCTAAGGATTAGTATTATGGGTATCTCCATTTCTAGAGAGGTAAAGTCACTCACA
CAGCCAATTGACACAGCCAGTGAATAGCAGAACTGGATTTGGCCTGAAAGACTCAGGCTCTGGAGCCAGTGCTCCTAAAGGCCATGT
GACATTGCTTCTCTTTTCTTTCTCTGGGGTGTTCCAGTTTTCCTCAGGGCTTCGTCATTGCTGCTTTCTACTTGCTGACACTTTTGT
CAAATGTGATAAACAGCAAAAATTGATACAGTACAGGTCCAAAATAAACGGACCCTGATAGGTACCACAGGTAAAGTTGTTGGCAAA
TGACATCACTTATGTAATGGTAGGTCCTTCACTGGAGGTCAATTATGGAGCGATGCTGTGAGGGAAAGGTAGGTAGATGTCTCTGGG
CTGGGAGAAGGCAAATGATGAGGGGACTTGGAGTTTCAGGGGGGGAAACAATAAAGATTCTGGTTGGCAAGTTTCTTGGCAAATTTT
AAGGGTGGTAAACATTTTAAATCAGTATGTCAGTAACTTCTGCATGAGAAGTGCCAGGTACTTCACAAAACTTTAATATAATGTGTG
AGCCTTAGGGTTGAAAGTAGGGATGGCACTGCTTCTGGCTTCTCTGTTAACAGAAATATGAGAGAGAAGTATGAATAATTTTTTCTC
AAATTGAGTTTTAGATTGGGGTTTCATTTTTCAGCAGAATATAAAACAGTTATTTTCTATTTCCATGAATACCAGGAGGGGAGACTT
AATAAAATGCAGTAAATATGGCCGGGTGCAGTGGCTCACACCTGCAATCCCAGCACTTTGGGAGGCCGAGGTGGGCAGATCACCTGA
GGTCAGGAGTTCAAGATCAGCCTGGTCAACATGGCAAAACCCCATTTCTACTAAAAATACAAAAAATTAGCCAGATGTGGTGGCGGG
TACCTGTAGTCCCAGCTACTCAGGAGGCTGAGGCAGGAGAATCACTTGAACACGGGAGGTGGAGGTTGCAGTGAGCTGAGATCGCAC
CACTGCATTCCAGCCTGGGCGACAGAGCAAGACACTGTCTCAACACAAACAAACAGAATAAAGTAATAAATAGGTGTATGCTGAAAG
AAAAATCATTCTTAGTCCTGGCCTTTTTTTGAGACAGGGTCTTGCTTTGTTGCCTAGGTATCATCACAGCTCACTGCAGCCTGGACT
TCCTGGGCCCAAGTGACCCTCCATCCTCAGTTTTCCAAGTAGCTGGGACTATAGGCACATGCCACCACGCCTGGCTAATTTTTTAGT
TTTTTGCCCAAGCTGGTCTCGAACTTCTGACCTCAAGCAATCCTCCCATCTCGACCTCCCAAAGTATGTTGTCTGGGATTACAGGTG
TGAGCCACTGCTCATGGCCCCATGACATTTTTTAAATCAAAATATCTAGATAAAAATCAACATATTCAGACAATCTAATTTGGTTGA
TATACAACTATGTTTTTAAAAATTTTCCCTTTTTGAAAAAGGCAATATTTGTGGTGATTGTAGACTTAGCAATGTAAGTTTAAGTTT
TGTAACATTGCAGGTTGATTTTGAGTTAATTGAAACATTTGAAGTTTAATATGTTGGCAAAGTGGATTGTGTACATGAATTGAGGGG
GAAAGAGGAAATTAATACTGTTTGATTTTTTTTTTAATTTTCTTGTAAATAATGTGAATATCCAGAAAGCAACAGAATAACCTAAGT
TATCAGAGGCTGAGAAAGGACATAAAATAACCTTTATTATTATTATTGTTTTTTTAGATGGAGTCTCGCTCTGTCCACCAGGCTGGA
GTGCAGTGGTGCCATCTCAGCTTACTGCAATCTGTGCCTCCCAGGTTCAAGCAATTCTCCTGCCTCAGCCTCCCAAGTAGCTGGGAT
TACAGGCATGCACCACCACACCCAGCTATTTTTTGTATTTTTAGTAGAGATGGGGTTTCACCATGTTGGCCAGATGGTCTCGATCTC
CTGACCTTGTGATCTGCCTGTCTCAGCCTCCCAAAGTGCTGGGATTACAGGTGTGAGCCACCACGCCCAGCCACTTTTTTTTTTTTT
TTGTGGGGTGGGGGGCGCAGATGGATGGGAATGGCATTGAATACAATATTCAGGATATTGTAGATGAAAGAGGAAAAGTTTCTAGAA
TATTATGTGACTGTTGTGGGAGGTGTTGAATATTATAAATAAGGAGATTTTGCTGCTGTTGTTTTTGTTTCTGAGGAAAAGAAACCC
TTGGATTAGCCCCAGTGGTTCTGGCTGGTGAAATTACCCAGTAAATAACCACAGACGTCTCGTTAGCAGAAAGAGAGAAGGGAATCA
GCAGAAAGAGCTGGGGAACAAGGTTCACTGGGGGCTTTCAAACTTCTCAGGTAGTATGTGTATTGAAGAGCTTAGATCTGAAAGATT
ACTATGTGCTCAAATACTTTTGTGAAAAATTCAGAAAGCCTATGTATAAATCTGAGAGAGAATGTGTGTGTGAGTAGCGATTATCTC
TGATGACTTACATATTTTAAATAATTTTCTATATTTTTTGTTTTCCAGATATTTGTATATATGGATCCATTTTGTTCATGTTGAGTT
TGTACTTTATTTTTAAAAATTTGCCACTACATTGTAAGCTTTCCCTGTGTCATGAGGTGTTCCTTGCAGAGGGGATTCACAACACTC
TGTTTCCTTCTCCACTCAGCGTGCTGGCTCTGGCTCTGGCTCTGGCCCTTGTCTGCACCTCTCAGCTCCTACACTCCGGAGTCTGTG
CTTTTCCTGTGGCCCAGAACATTCCAGCTGCCACCCTCTTCACCTGGCTAGCTCTTAATCTTCGTGGTGCCCTTTTCTTTTTTAGGA
GGACTTAAATGCATTGGACAGTGGACAAAGACCATTTAAATGGATTACAAAAAGCAGACTTCACCAGGCAGACCGGCCTCTAACTTC
TTCAGTAATGTTGCCATTTACCATGTGTCTGTTGGGGATCAGGTCCGTCGCTAGGCACCCTTCGTGCTTTTAGGTAATAGTCATAGC
TACCCTGAATAAGGGTGAGGAGATGGCATTCCCATTTAGAAGTTGAAACCAGGATTGCAAGAAGTGTATGAGCCGGGATGGGAAAAG
CCAGGTCTGTCTCTCTGCCCTTAGATCACGCTGCTGTTACTATTCTTTGTGACACCCTAGGACATTGCCGTGGAAGGAAGGGGAGAA
ACCTTGATAGGGCCCTTGGATGAAGCACTGCAGCTTATGCTAAGAAGATCAAGGCCCTGTTAGACTGGAAACATTCATTGTAAGACC
TAGTGCCATAAGGTAAAGAGGTCTCTAGGAGAATGCTCAAGATCTCTGAGGGATAACTGTTAAATATCCTGTAAGAATTAGGCTTAA
ATTATACACACGTGAGAAAGGTAAGTGCTCTCAGTGCAGGGGGTAAAATAAACCTTTAGTGAAACTCTATGACGAGGAACCCTGCCC
ATCAACTGTTGGTAAGTTTAGAAAAAATGAAGAGATGCAATTCAAGGTCAGTAAGTAGGTGAAATATGATGTGCGCATAAACCTTTA
AAGGTTTGAGAGGTTGGCTGTTTTTGGTAACGGGTTAACTTGAGGATTAATGAGTCAGGATATATGGAAAGTTAAAATTCCCTTCTC
TGCACCATTCAATTCAGTTCAACAAATACTTTCCCAGGCCAAATGGGATCATTAGCCTTGTAGGAAACTCTTGGCCTTTTTAAAATG
ATGACATTGTGGCCAGTTTTGTAAATACAGTTGATGCGTGAATCTGTAGACCAAGGATCAGATTAATTTTGTAATGCCCTCAGGGAA
GGCAGTTGATGAACTGGTTTCTCTGCATCATAAGAGCAGTGTTAATGATCCACTTGTACCATGAGATTCTAACATTCCTGAGTTACC
TAAATGCTTCAGTGTTCCCTGCCATGTAATTTTAAGCTGTATGTACATTTTTGGGATAATTTTTATCCATATTTTGTTTAAAAAATG
AACTAAACCAAACTAAAATTCAGAACATTTTGTACTTCTGCTTCTTATTAAGATTGTTTAGTCCTTGGTGGGTGATGTGAGAAGATC
AGTACTTAACAGTTTGAGAATTCTTGCTCTCATAAATCAAAATAGTATTCGATGACAGTTTTCAAACAGTTTCAAGTGATGATTTAG
ATTCTCTTGTAGTTGTTATCTACTTTAGAATAGTGTTACTATGATTTAAAAGTTGGTATTTCGGCTGGGCCATGGTGGCTCAAGCCT
GTAATCCTAAAACTTTGGGAGGCCAAGATGGGAGGATCACTTGAGCGCAGGAGTTCGAGACCAGCCTGGGCAACATAGACTCTATCT
GTACAAAATACAAAATTTAAAAAGTTGTTATTTCATGAGTTTGCATTTTTAAATGCAATGGACAATAGATAGAACTGTTTATAAGAT
ATATTTGCTTGTATGTTTTTCTCTTCATATTACGAGGTCAAACCACAGAGGAAAAATAAAATTTGGAGGCCCTAACCCTATTTACCC
AATGTCTCCTAGAAGAGCCCCCTTCAGAAACTTCTAGCAATTGTTAGTGTTGCTTGGGTTAATAGCCATGGTCAAAGCATGAACAGA
ACAAAATAAAAATTTTCTAAATATAAGGAAATATGGTAATAACTGAGAAGCCAAGCATTAATAACTGGGAAGCCAAAGCATTCTCTC
AATATCCTCTTTCTCCATAAGAAACGTGTAGCATTTTCCTTGGAGAAGGGTCTATGTTACTTTTGTTCAGCCCTTGATGAAAACAAC
ATATGGTTAGTTTTATGAGGACTTAGAGACAGATGGTGACCTACAGGCTAATTATGCAGTGGGCAAGATTCTTTAAACAAATCACTG
TGATTGTGACATGGCCCTTTCTCATGAGTTAGACAATCTGGACTGTTCATTGTCACACTAGGTAAACATGTTCCAGGTGAATGAGGA
AGAGGTGCTACGTGAGGGAAATGTATTTACAACATCTTTTCATCTCCATATTTACTTTAATGGGTATAAGAGAAAAGGAAGTCAAGA
AGGTAAAAGGGGAGCACACTAAAACCTCAGCTAGTGCTAGGATTGTGTTATTTTGGACAGCTAACTTTTCAGATAGCTGAGGGGTTC
CCCCATTAAGCATAAACGTTTTTCTTTTAATCACTTTGAATGAACTCTTTCTGAAGTCTTCCTTGCCCTCTGAAAACAGAATGTCTG
AGACGTAGTAGGCTTTATAGTGTTTATGATCTTACTTTTCCTGCTTTCCCAAACGTAAGCTTCATGAAGGTAGCGGTCTCAGTCTGT
TCTGTGCTGTGACCCTGGCCCCTAGAACAGTGCTGGTACACAGCCACATTAAATATTTGCTGAAGTAAGTGGGAATTAATTAAAGCT
CTTTAATCTTCACAACTGTGTCACTGTCCAGGCTTTGTTCTCCCTGTTTGTAGATTGGTCAGGGACCCTAGAGTGCATTGTGCCTAT
GCTTACGGTCTTAATGTCCCTCTGCCCCAGTGTGGTGGCTCATGGCTGTAATCCCAGCACTTTTGGAGGCCAAGACCAGCGGATCAC
TTGAGATCAGGAGTTCGAGACTAGCCTGGAAAAAATTGTTAACGGCGAAACCCCGTCTCTACTAAAAATACAAAAATTAGCCAGGTG
TGGTGGTAATCGCAGCTACTTGGGAGGCTGAGGCATGAGAATCACTTGAACCCGGGAGGCAGAGGTTGCAGTGAGCCAGGATCCCAC
CACTGTACTCCAGCCTGGGTGACAGAGTTGAGACTGTGTCTCTAAATAAATAAATAAATAAATAAATAAAATGTCTCTCTCATTATA
TTGTAATTGCCTTTTCTTGTCTCTCTCCACCACCGGACTGTAATATCATTGACAGCAGAAATTTTGGAGGTTCACTGTTATATCCAC
AATGTCTGGTAGTACATATTGTTACTTGTTTCTTGAACGTGTGAACAAACTAATTCACCAAAGGAAATGGTCACTGCCCTGGGTCCT
TTCCTTTACAAAATCGTATGATGTAGATAGTTTGATGCCCATTTTACATACACACAGCTGAGAGGAGCAGCCGTGATTTGCATCCAG
GTTTGTCAGCCTCAAGGCTAACCTTCCACCGTATGGGTAGCTCCTCACCTTTACACACACAAGTGCCCCCTTTCTGATGTCAAATTT
TCCTAACCTTGCACAGTATGTGGTTTTTTTTTACTTACCTCACCCAGAATGGTATAATAAAAATGCTGTTATCAGTTTAGTAGTGCT
TTTAGTTACTGTTTTTAGTCATTATACTAACATTGTAAATTTGAAAAAAAAAAAAAAAAGCCAGAACGGTTTGTGGGAGAACTTCTT
GTTGGTTGCAATGCTCCAGGCTGCCTGGGGTTTGCCAGCCACCCCCCTGCAGTAACCCTGTGCACTTCCCCTTTCCCCAGACTCCCT
CCTCCCCGCCAGCAGCCCCTTCTGCGCTCCCCCGTGCAGCCTGCCCTCCATGAGTGGCCACGGACTTCAGGGTCCACGCGGTTCTTC
ACACCGCCCTTTATGTGATGTGTGTCCCCTCCTTGGCTGTCTGGTTTTGCTTTTGTCAGTAGGGCTAGTGGCCAGCCCATCTCCATT
TTGAATTTGCTGTTTTATAATAAGCCTTTGATTTGAAAGCCAGATGATCAGGGCTGCTAGAACTTGCTAGAATGAGGAAAATAGACA
AGTGGTCCATTGACCTCTGAGTGAGTTACAGTAACTTTTACTACATGGATTTCTTTTTTTTTTTGAGATAGAGTCTTGCTCTGTCGC
CCATATTGGAGCGTAGTGGCGCGATCTCGGCTCGCTGCAACCTCCACCTCTCAGGCACCCTGAGAGGCCCTGTTCTCAGGCCTTCTC
AGGGTACTGGTAGGTGAGGCCCCAGCACGCGTTGCTCAGAGCCAGTTTGGCTCAGGTCTGTAGGAGGTGCGATGTGGACATAAGTGG
GGTGGAGTATGAAAGAGGAGAGATGGCAAAATGGGAGAGCGTTTACCTGGTCAAGGGCTTAGGTCACATTAGGTTCTCAAAGGCAAA
TATGGCTTCATGTGGATACTTTCTTGAGTCCATCTCTCTCTGTTTCTGTTCCTCCCTCTGCCTAACCTATCCTGGCTCTAAGCTGGC
TCAGACACTGCGTTTGTGTCTTTTCACGTGGCATTTTTCTTTTTTCCCTCCACCTTGCTAAGTGCTGAAATTTGCAGTCAGCATTTA
TATATGCCCTATAGACAAGGCACATTTCTCTAGTTTTACCTTTCTTGTCATTTGTGATCTTTTAAATTTTCAGTCAATGGAAAGTAA
TTGTTAATATGAATTGCAGTTTGGAAACTTTGTGCCAGATGATGAAACTGAATGAATCTGTGGTCTTTACGTAGGCAGTGCCTTACG
TATGGTTTGGTTTTTGGCCCATGTACTAGATGTTTGCCTTTCTGTGGGCTGTCACAGATCTGGCATGTACCTTTATTTTTTTGGGAG
TTTCTACATGTGTTGCTGCAATCTCTCAGCTAAGAGTATTTTTCAGTCCTCTTGCTATTTGTTTTTTACCACTTATAGGGTGAAAGG
AAGTAAAATTAGATAGGATTTTGATGTTTGATTTAAAAATCAACATTTCCATGAATTCAAAACTTCAAGAAGATGAAAGGATAACAT
TGATTCTTCTGTTTAAAGAAAATTGATTTCCTTTGGCTGAAAAAGTTTTGTTCTCTCTCTCTTGGTGTTCATTATTTTAGATGTTCC
CTTTGGACTCTGTGCACATCCCCATCATAGAACCTACTATATTTACTATGATAGTAAATTGACCTCTCTGCCAACACCACTGGACCA
TGTAAGTTCCTTGAAGGCAGGGGTCCCTAGTGACAGGCAGAGTAATATCTGGTACATTGTAGACACTCAGTAAATGTTTGTGAAATG
AATAGAGTGACCAAGCAATCTAATGAATATGGTCCTATCATGGTCCCAACCAGTCATTGGACTGAGTATAGTTTTGATGGTGTCATC
TCAAATGAACATATCTCAAATCACATCCAGGTTCAGCAAATTTTCTTCTCTGTCTTGCCAAGGTGGGGGCCCCACTCTGAGTCCCAG
AGATAGAAACTCCAGTCATCTTTGCATTTCTTTTACTTCCTTAGTGTCTCTTGTATTTTCCTCTGTTTTTCTCCATTCCTACTGCCT
CTACTCTAGTTTACACTGTTGGTCACCTTATGTCTGTAGCCTGTGAGCTAGCTTGCTGACACGAGCACCCCCAGCCATCCTGCACTC
TGCCGCAGCCAGATCAGGCTGCCTCCCACATCCCTTCTAATAGGACGTCCCTGCAGATGGTCAGAGTGGGTTTTCCCAAGGAGTTCT
GCAGGGCGGTGTGTTCCAGTGGACATCCTTGGTCCCCTGAGGACCAAGGCTTAGAAACTGATTTTATGAGCCTAGCCAGAGCTAATC
TCTCCATCGTGTTATCCCAGTTCAGGAAGTGCATATATAAAACCAGATTGTCTGCTTACTGCCTTAAAATGAGAATAGAACTGTTTC
TATTTCCATTTCATGGCCTGGGATTCTGGTGTGGGAACATCTAGTACACATTTTCTTCCTGTAGTCAGGCCTTCGTGGGAAGCCAAA
TCATAATCAGATGCTTACTTGGCTGGTATGGAATAAAAACAAAGAGAAGTGAAGTGTCAGATGGCATCAAGGTTAAGTCCGAGGCAT
CCATTTCACAGTCAGGAGGCTTGCCAGTGGGAATACCCCAAGTGAGTTGGCTTCAGTTGCCTGCATCCTTCTCCACAGTGAGGCTGA
CTTTGCCAGTGGGTCCTGACCCTCCAAGGCTCAGGAGGTTGCCTCAGGCCCTTTCTCACTACATATTGCTGGGAGTGGCAAGGCCAT
TGCTGTTCTCTTTGACTATCTGCTTTCCTCCACTTTCAAATCTGACCAAGCCCTTCTGATTGACATTCTTACCCTTAGATATAATAC
TTTCAATTCTTTAGGGCTTGGAGCTGTTTCTCCAGGATGATTTTGCATGTCTTGGCTTTTTAAAGTTTAACTCTAAGAGCAGTATGC
TATCTTTATTTCAAAGCCTTTATATCTTCTCTCCCTCACTCACCATCTCCTTTCCTCAAAACCCTTTTAAGCCCTAACTGTTATTGG
TGGGAGTCTAAATTTCTTTGCCTAGCCTCTGAGGTCCTCTGTCTGCCTCCCTGAGTGATCCAGACTTCTTTTCTTTTTTTTGTGGTG
GGTTGGTGGTGGGCTGGGGGTGGGGGACTGTCGCCCAGGCTGGAGTGCAGTGGAGTGATCTCGGGTCACTGCAACCTCTGCCTTCTG
GGTTCAAACGATTCTCCTGCCTCAGTCTCCCAAGTAGCTGGGACTACAGGCACGTGCCACCACGCCCAGCTAATTTTTTGTACTTTT
AGTAGAGATAGGGTTTCACCTTGCTAGCCAGGATGGTCTCGATCTCCTGACCTCTTGATCCGCCTGCCTTGGCCTCCCAAAGTGCTG
GGATTACAGGCGGGAACCACCGTGCCTGGCCTCCAGACTTATTTTCTACTTGTTTCCCTGTGGAATAAGAGGACTGGGTCAGAAGCC
CCAGCATAAGTCATTCTGTCTGTCTGCTGGAGTTTCCTGAGCTCTAAAAACCCATCTTGCCTATCTCATAGGATTGTTGTGAGGGCC
AAATACAGTGACAAGTGTTCATTTTCTCTATTGGTGCAAGATTCTGCGTAGGTGATGTGACTCCTTTTTTTTAAGCTATCATATATT
GAGCCCAAGCAGTGTGCATGTATTATTTCATTTAGTCCTTTCAAGTAGATCCTGTTGTCATCCCCATTTTACAGATGAAGAAAGTGA
AACTCAAAGAGGCTAGAGAAGTCTCAGGACTGATGGAGACCAGATGAGTCTGTTTAATCCCTTTAGTCTGCTCTGAACTTCCAGTAC
CCACGGCTATATTGGACTGAACACTCGCCTGACCAGGCCCCCCGCCCCGCCACCACCGCCAACAACTAACTGAACTCGTTTCTACTT
CTCTGACCCACTGCCCTGGCCAAAGTCCTCCCTGCCTTTCCAGGCCCCTTTCAGGTTTCCTCTCTCTCCTTTTGTGTCTAGCCCTCA
TTGCTCACCATCATCTTCTCTGACCTCCTGTGGTACTTGAGGACATACTGGAGGCAGACCCATCTGCGATTACATCCCAACCGTGCT
GTGTTGCTTTGAACAAGGCAGCCACATCTCCGAACCACTGAAAAAAGGCATAATAATGATAGCTGGATCCTGGGATTTCTGGATTCT
GTAAAACCAATTGTGTAAAAGTTCTTTGTAAATGGTTAGATGTGCCTGGTTTCTTCAGTAATCAGTTATTTTTGAAATTATTTTTAA
TTTGTCCCATAGTAAGGAATATATTTTAATGTTGTGATGGGCACATACATTTTAATTAAGAGTTCCACAGGAACATTTTCTTCTTTT
TTTGTGTGTGTTAAAAACAGTGTTTATTTTGACCCATTTAATTGATTTTGACTATCCACTGTAGGAGGCTACCCACAATTTGGAAGG
AACCATCGGGCCTAGATGACAATAAACCCTTTGAGGGCAGGGAATGTATTTTGCACTTCTGCGTCCTCATAGGCCCAACAGGTTACG
AGAAGATCCAGTCTGTTGATGGATTTTGTGCTGGGAGTAGGCAGGATGGAGAGACTTATTCAAATTTCACATTATTTATTCAGTGCC
CCTTTGCAAGAATTGGCCAGTTAAACCCTGGGGGGCAGCTTTGTAGCTGCACTACTCGGGAGCCTGCACCCTCTTGAAGACATCATG
CCAGCGTCTCCTGCACTTGGCCGTGTAGGCTGCAGCACAGCTGTTTCTGCTACAATCAGCTCCAGTATTTGTATTCCTCCTCTTGAG
GCCGAGGCTTGTTCGTTGGCCCCAGGGAATGTGTTGTGTTGGGGTTTGAATCTCCTGTCCTGCGTGCGGTGCCCCTGCCAAGGGCTG
TGCACCCACCTGTCCCAGATGGGTCTTGCGTCATGTTTTCCCTGCTATGCTGGTGGTGACAGCATCCTGCTGTCTCAGAGTAAAGCT
CAGCTGCATATATGCCACCCGCCCTCCTCCACAGCATCCAGGCTTCCAGTGTAAAAGCCTCTTACAACATCAGGCGGGAGAAAACAA
GCCTGCCGCTTGTGGTGCCTTAGCACCAATTTGTCACCCACTTCACTCAGCCCTGGCCCCCTGCTTCTCCTCTGTATGGTATCTTTT
TTTCCCATGGCTGCTCCAGCTGGGCTCTGGCCTGACACTGGAATCACATGTTTTGGCTCTAGCCTTTGGTATTATTCTCGAGGTTGT
CAGCATACATCCCCACTGCCATCTTTTTCTAATGCAAAGGTCTTTTCATTAATGGATATTTTTTCCTGTGAGACTTGTTTAGGGGAG
AGATGCATCCCTCATTTCCCCACCCCCACCCGCCATAAGGTTTTTACAAGCCAAGCTAGAGATTGCTTTCCAGCCACTTTCAGAAAA
ATGCTGGGGCTCCACTTTGTGTTTAGCACAATCAGCACACATAATTCTTGTTCTGCTTGGGCTCTGTTCAATGTCACTTGCTCCTCC
TGACCCTAGAAAGAGTTGAGAATTATAAGCACGGATTATAAGGACTCATAATCTCAGTTGCCAGAGTAAGGGAAATAACGTTTTCAC
CGGTTCCCTGAAATAGACCTGGCGGACAGCTGTTGCCTCCCCTCCCCTTTCCTCTCATTTCCCCTGGAGATGTACTCTGCCTGTTTC
TTCTCCTCGCTAAAACTAAGGAACTTTCCCTTGTGTTTTCTTTTGGCTTCTAGATTGTTCACTTGGCTTTTTATCTAAGCTTGTATT
AAATTGCTTCATGATCTGTGCCTGCAGAGGCTTTGGCATAACATGGCTCACAGAGAAGTGTTGTTTGTTGTGGTTGGACCACTTTGT
TGCAGCAGCATCTGTTCTTGTCTGAATGGGGCATGCAGGTGAAGCATGTACTCTAGGCCACTGAAAAAGGAGGGAACACAGGCACCT
GGACTGACCATTGGCAGCTGTGATCTACTTATGTGACTCTCAGACAGCCTCTCAGCTGCTTGTGATAAAGGCTACAAAATTCCAGGC
AGCTTTTTGGTCCCCATAACAAGTATATATAACTCCTACTTTAAGCAGTCCAAATTTAATACAGAGCAATGGGATGAGATGTATGTA
GGTGGTGTCACATCCACAAGAATATCTTTAATCATTATTCTTTTCAGCAATTCAGTTTCAAGAGGAATAAGTGAATTTAGGAAATTT
GTATATGGGAAAACATTGTCAATCAGTAAATGCGTGGTCTACTGTGTATATTTTCTCCTCAGTTCTTTAAGGCCCCCAAAATGGGAT
GAAAATTTGCTCAAAAGCTCTCATGCAAAATTATAAGAATATGGAGATATATTAGGAAATGCACAGATAAATATGCTTTCAAGAATC
TTATTAAAGTGCTCAACAGTTATATGAAACATGCAAGTATAAGTTGAAAATTTAACTGACATGTGGACATTGAATGTTTCTGTGATT
TTAATGAAAAAAAATTCAAGGAACAAGGGGGAAAAACACCAACCGTCAAAAGGAAAAATGCCCTCTATCTACTCATGCAAATAGTCC
CCTTGTTCATAGCAGTCACAAAGGATATGACGAGCCATATTGTGCAGGATCCAAGGTTCATGAGAAGTAGGTTTGTAGGTGGCAGGT
AGTGGGAAAGTGATTTATGTCCCATGAAAACCCATTCAGCTGTTGTAGCACTTCCAGGGCCTGTGACGTGTGAGGGAGCCATTGTCT
TAACACTCAGGGTGAGGACAAAAAGGCTTCAGTTGCTGAAAGGTGCTGGTTTGAGATGGTGATATCCTGTCTCTTTTTCCAGCAGAA
GCAGGAACATGATGTCTAGGGTTTTTAAAAGAAAAAGCTTTTGTTCTTTTTTATTTAGTGACAGTAGAAATTAAAGTAGGGCCTTAA
AAATTAGAAACACACGTGAACCCCAGGGGGCGAAGCCTGCAGTGAGCCGAGATCATGCCACTGCACTCCAGCCTGGGTGACAGCAAG
ACTCCATCTCAAAAAAGAAAAAAAAAAAAAAATTTAGAAACACAGTCAATTTTAATGTCAAGACCCAAGCCTCAGCAGTTAAATCGG
ATGCCTCCTTAAAAAATGTGTGAAAGTTATTTACCCTCATTTCAATTTGGGGCAACAAATGAATGGCTACTTAGGTTAGGTGTGGGA
AGCATTAATTAATAATGGGATAAAAGGACTCAGTGAGATTAACTTAGATCACCTATATTTAGCTTCTGATTTTACTTCATTCTGTTT
CCTTCTTTCATTAATCTAAATTAACTGCCAGAATTGCATTTATTAGTATGTAGGAGAACAATAATCAAGAAGTGTTTTTATTTATAT
TCGTAGGAGGCTATGTTGTTATTCATGTGATTCTTAGAAAATAATGGATTTTACACAGGAAAAGATGTTTATACTTTTCAAAGATCT
TACTCATTTGGGGTAAGAATTTGATAGAAATGCTAGGATTTGGTGTATAGAAAAGCATGTAAACTGTATGTATCATTTAGGTTTCTA
ATGCCTGTTATTGGCTTTCATGTAATTTGGATTGGGAGTCTAGAAGGAAGGCAGTGATAACAGGAGGGGCTGGGGAAAAGTGAAATT
GTTGCCCATTTCTTCAATATAGTGTGATAACTGTCTCCCATTTAATTTTGTCTCATCACACGTTTTTTGTGTGTGTGTCTGCTTTTT
GCCAAAAAGTACTTACTGGTTCTAGGCCTCAAGGAGCTTATGATTTAGCACAGATTTTCTCAGCCTCAGCACTGTTGACGTAGTGGG
CCAGGTAATTCTTTGTGGTGGGGACTGTCTTGTATATTATAGAACATCTAGAGACTTCCCCAACCTCTACCCACTAGATGCCAGTGC
ACCCTTCCCCAATAGGGTGACAACCAAGATGTCTCCAGATGTTATCGAATGTCCTCTCAGCGAGGCCCAGAATTGCCTCCATTTTGG
AACCACTGGTTTGACACAGCACGTCTTGCTATAGAAACTCACTAGCATTCTTTGCCATTACAGCTGTTCAGTTACTTATGCTTATTT
CTAGTTTTGGAAAAGCCATTTGAAAATGCCCTGAAGTGATTTTTATTCCATTCTCATAATTCTTGAAAAGTGTGTGGAAAGCTAGAG
GAGAACTGTTAACTGTGTCTGTGTGCATGTGTATGTGTAGGGGTGTTTTCATCATTGTCATCGTTATTTTATGAAGAAAATGGAGGC
CTCAAATGTATGAGCCAAAGGACAAACACTGAGATGTCCATGAGAAAGCAGGGCGAGGACTGCTTTCAGGAAGTTCCTGATATTTCC
TGTATTAACACAATCTGTTAAGACCCTGAAAGCTTAGGGCTTAATAAAATCGTTGAGATCTTAAATGTAGAGGATGTAAGATTTTTT
ATAGATCAGCTTTAAAATGAAGCCTAGGCAAAAATGAAGTAGATTTGCCCCTCTTCACCCTCACCTGCCTTCTCCCGGTTTCTTCTC
TTTCCCTCCTCTCATCTCTGCTTGCTCTGGCAGTTCCTTTAACAAACATATTGTTATGTGATGACTTTGATGAATTACAAACAGGTA
CCTCTCTTTCAAGATACATTATATTCATCTACCAAAAAATGGTCGTAATAGCTGTACAAAGCTGCAGTGACCCACGGTAACTGGAGC
CCCAGGAGTTGTGCAGTGCACAGCTTGCACAGAGTAAGTGGTGGCTATGTTTGGGCAGTTTCATTCCCTCCCATGACATTAAGCTTT
CACCTGAATGCTGTTCTGTTTTTGACCCTAGACCAAACCTTTTCTGGGAACATCATGCCTATGTAAGCTACCTTTTGGACACATTCT
TCCTAGAATACTCTAAAGCATCCCAAGTGTAACATATGCAAAATTGAATTACTGTTTTCCCCAAGCCTGGTTTTTCTTAAATTTAGG
AGTCGTCTTAGTCTCTCTTTTTATATACTTATATACACACACTATACACATACACATGTATGTATCACAAGCCTTCTCAGTCCTCCC
CGTATAGTTCTTAAATCCTGTTTTTTTGTTTTGTTTTGTTTTGTTTTGTTTTGTTTTGTTTTTTGAGACAGAGTCTCGCTGTGTTGC
CAGGCTGGAGTGCAGTGGCGCGATCTCGGCTCTCTGCAACCTCCACCTCCCAGGTTCAGGCAGTTCTCCTGCCTCAGCCTCCTGAGT
AGCTGGGACTACAGGCATGCCCCACCACGCCCAACTAATTTTTGTATTTTTAGTAGAGATGGGGTTTCACCATGTTGTACAGGATGG
TCTTGATCTCCTGACCTTGTGATCCGCCTGCCTTGGCCTCCCAAAGTGCTGGGATTACAGGTGTGAGCCACCGCACCCGGCCTGTGA
TTTTCTTTCTACCCCTGCTTTCCTCCTGTTGTCTTATTTCAGTTTTCATCTCCTCTCTCCTGAGGCCTGGGCCATTGCAGTCATCTA
CTACCTGCTTTTCCTGTCCAGGTATCACACCCCTTCAAGCATGTTTCAACTAGAATGCTCTTGTAAAACACAAATCTGGCCTTACTG
TTTCCCATCAGAGCTCTTTGAATTCCTTCTGTTATGCCATAGTTTATGCCTCCTTTATAGCCTTATTTATAACCTCATCCCCTCCCC
TCAAATGTCTAGGCATTCAAAATCTGCAAAGTGCAGGGCCTGTTCTAGGCACTGGAGACTCAGCAGTGAATAAAACAAAGTCATTGC
CATCATGGAGCCTACTTTTGAGGGGGCTGGACTATTCGCAGTTCTGTGAGCTGCCATGCTGTTTCGTACCTTTGCTGGAATGTGCCT
GCCCACAGCTAATCTTCCTGGACAGTGTCTGCTGGAATTTCAGGACGCAGCGACGGTGTAGTGACCCCACTAGCATTACCACTCTTC
TTTTTATGTCTTCCTTGTGTTGTGTGCTTCAAGCTTCCTCTTCACCAGGGCCTGGCCTTACCAGGCTTTGTGTTTCCAGCACCTATA
GTAGTGCTCAGCGCATAACAGATGCTTCTAAATCTTTATTGAATGAAAAGCACACAAGCATGGAAACACTTTGGGATCCGTGTCCCT
CAAATGTGTCATGTATTTTCTTCCTTTCTTTGCTTATACGCTTTCCACTGCCAAGAATATCCACCCCAACCATCCTACCAGGCCCTT
ATAAGCCACTTGCCCCATTCTTTAAAGTGTCGCTGTCATAAACCCCTGCTGAACCCTTGCCTCAATTACTTATTGACCTACTTGCPT
TTTTCATGCTTTCAGTAGCATTTATTCAATAAATACTGAATATCTACCATTTTCCAGGATTTGTGCCAGGTCCTAGGGATATGACAA
TGGAAAAAGATCTGCTAATATTGTATTTTTAGTAGAGACAAGGTTTCTCCATGTTGGGTCAGGCTGGTCTTGAACTCCCGACCTCAG
GTGATCCGCCCACCTTGGCCTCCCAAAGTGCTGGGATTATAGGCGTGAGCCTGGATGACTGTCCTTTTTGCAGTGATGTATAGGCCC
AGGCCCTTTCCACGTTATGGCTCTGCTAGCCCCATTGTGTTCCGTTTTATGTGAAAGGGTAAAGAAGAGAGCATGGAGTGGATATAT
TTGTTTCTTAAGCCTTGCCTCAGAAGTGACACCTACCACTTCTCTCACAGTCCATTGGTAAAGACAAGTCACAAAGCCACTCAAAAC
TGCAAGGGCAGCTGAGACATGTGGTCTAGCTGGGTGACCAGAAAGACAAAATGGAGTTTTGGTAGACAGCTACCAAACTCGGCCACA
TACCTCATTATACTAGCTGCAAACTGTGTTATCGTTAGTTGCCTGCATATTAATTAGTTCCTTCTGCTTAGAACACTTTTATCTAGA
ATATTTGCAACATTTGCCACTCACTTCATTTAAATCTCAGCTCATCTGAAACTTCTTCCAAGTGTAAGACCACTCAGTCTGGAAAAG
CTCCCCATCTTCCTACACTGCCTTTTTTACTTTAGAGCCCCTGTCACACATTATATTATATATATAGTTTAGTTTATTGTGCTCCCC
TGCACAGGAATGTAAGTGTCATGATGGCACAGCATTTGTTTTGCTCATTGTTCTATCTACAGAATCTAGAAGAGTGCCTGGCACTTG
TCTTCAAGGATTATCCACATCCTGAAGGCAGAGCTTTGTTGTTCACATTTCAGTGAATATTTGCTGAGCGGAGCCTCATGCTAGACA
GGCCTTGGAGGCACGTCCTAAACAAGGAAAACACGCCCCCTGCCTCCCTCAATTGAAGATATTAGTCTCTTCAATCTCATTCAATTT
TATTTCTCTGTGCTTAGCATTTAGAAGCAATTAAATATGTTTGTGGAATGAACAAACTAATTAACAGCATATTTAGAAAATCTCCCT
GTAGTAAATATATTAAAGTTTTAGAAATCATGATAATCTAAGTGAAAATATTTTACAGATTATGGGGAAAACATGAGGGATGAAAAT
TTACCCTGCTCTGCTTTAGCTTTTGAAAAAGTCTGCTTATGAGCCACTCTCTTCATTGCTAATAATAACACCAGTGGGGGAAACTTG
AAGAAAGAGGTATTTGTCACCAAATGGTAAGTTAGCAGCTTGTCTTGTTATCTGGGTTGGAACACAAAGTCATTAAGTGGCATTTGG
ACTAAATCTGATTGGAATTTAGAATCTACTTGGAGACAAACAAAATATACTTCTGCTTAGAAAAATTACATTTTCCACTTTAAGATG
TTGTGGATTAGAAAATGATAAGAATTAATTTTAAGAGAACGCCTGTTTTTTGAAAGGATTGGTTATATGTTGGTGGAAATTCTTCAG
GGAAGTGACATCTCTGGAGGGGGAAACACAAAAACAGCTCAATACAGTCAGTCAATTTATTCTACACTTAAGGAAAAAATAACTAAG
TCTGCTTGCTGGTTTTCTGTGGGCTGTTAACAGCTGGTTGACTCAAATTTCTGCTTTCTACCAATTGTGAAAATTAAGCTTTAATTT
AGAGGAGCACCTGGATGTTGAGACTCTGTCTGAAGATACAGGGGCTAAAAATGTTCAGATTCTGTCTGAAGATACAGGGGCTATGGC
TTGTCTATGGTTACAGTTTTTGCAAATGCAGAAAAGCTTTTCGAGCCTACTTATGTTATGGAGTATTTCCAATAGTGGTTATTAAAG
TAATTCAAGGAAAATCAGTAACTAATGATAATAAAAAAGTTGCCTTAAAATAGTGTTCAAGAAAATAGAGTGTGTATGTCATATTTT
AGCACATAGAAAGGCTTAAACCCTCTTGTGAACTCCTACTTAACCATTTCTTTATTATACTCTTCTTACTCAGATTTATAAATTTTC
CTTCCTTTCTGCAAAAAGACAGAGATAAATAAAGATACTAACTTTTCCCCGTCATTATATCACCCCATTACCCTGTTATGACAGTAA
TATTCATGTTGAATATCCGGTCATGTTTAGCCATTTGTATTGTGTTTAAAGTTATTTTTTATGATTTCCAAATTTGAAATACTGGAA
AGGAAGATCCCAGCCTGATGGAAATTTTTACCAGAATAGTGATTCCTTTTTGCTGTAGAAAACATGGGAGGGAAATTGTCTTAGTGT
CGTTACTGTCTAGTAATACTGTTTGAATTCTTGAAAGGCTTGTTTCCCACGGTGTTTTACTGCATTATATTCATGGAAGGGAACATG
CAAGCCAGAGACAGTACAGGTTTTAGGCAAAGAGGCATGGGTTAGGATTCTGCTCTGCCACTCTGAAGGGAGTTATTTCTTCTGGCT
TATTCTGTGTCTTTGTTATTTAAGTGAGGGCATTTACAGGACCTACATCATAGAGTGCTGGGAGATTAAACAAAGAGACATTCACAG
GTTATGCCCTGCTGCTAAAGAAAAGGAATTACCATTTTCAATTTGTTGTCTAAAGACCATCTGAAAATCTTGCTGTCCCTCAGTTAG
ACTCTGGCCTATCCACAGTAGGTCAGCTTGCCCACATGGGTTTCAGCAACTCCACTCTGCTCTGAGGCTCGCTAAAGGGTGAACAGG
TGATCATGAATGTGGAAGGCTGAAGAAGAAACTGGAAAAAGCTGAAACTGAATATCAGCATTTAGAAACTGCATTTGGAGTGCTGAG
ATAATAGAAAGCTTTCAAGGCACTTTGGAACTCTACATATGTTAATCAAGAGCCTCAAAAGCAGCCCATCGATTGGCAAACCAATCT
CTTCAAGTGCCACCACATTAAAGTCCCTGGGGAAGCTGTGGATATTAGCTAGCAGCGGTGCACAAGCCGTTTACCCTCCCTTCGCTG
CAACTTTAAAAACTTTTTGCCTAATAATCTCTGTTCTAAATTCAGTATGGATTTACTTAGCCAGCAGATTGACATCCAGGGGGACAG
TGTGAGACTTGGAAGAGTTTTATATTTACAGGAAAGTTGCAAAGATAGTACAGTTTCCATTTACTGTGCACCCATTTTCCCCTGTTA
CTAACACCTTATGTTAGAAATGCTTGTTCCCTGGTGCTGCAGAGAAATAAATGTATGAGCATTCGAACATACATTTAATAAAAATGG
CCTTGCTAAGATGATTAAATTTATGTTCGAGTGCTGTTTCTTTGCGTCACCGGGGAACAAGTATTTCTAACACCTTACATGTTAATA
TACTGCATTTGTTACAACTAATGAGCCAATATTGATATATTATTATTAACTAAAGTTTGTACCTTATTCAGACTTCCTTAGGTTTTA
CCTAATGTCCTTTTCTGTTCCAGGATCTCACCCTGGGCACTACATTACGTTGTTCATGGTTTCTTAGGCACCTCTTGGCTGTGACAG
TTTCGTAGACCTTTTTGTTTTTGATGATCTTGACAGTTTTGAGGAATAGTGTTTAGGTATTTTGTGGAATGTTCCTCAGATTTGTCT
GTTGTTTTTCTCATGATTAGACTAGGGGAAGACCCACAGAGGTTAAGTGCCATTTTTGTTGTATCAGGGGTACATCCTGTCAACATG
ACTTATCACTGATGTGATCATGTCAACATAATCTTGATCACGTGGCCGATGCAATCCGCTGCCAGATTTCTCCACTGTAAAGTGACT
CGGCCCACCTCCTTTGGAAGGAGGCCACTACGTACAGCCCTTGCATAAGGAATGGGGAATTTTGCCCTTCCCAGTTGTGGGCAGAAT
ATTTACATAAATTATTTGGAATTCTCAGTTGTATTTCTAAGTGTGTGATTTTAAAAACTACTTGGATGTTAAAATGGTTACTGTTGT
ATTCATTATTGAGTGAATACTTACAAATGTGGTCTGATGAACATTTTATTCTACCTAAGGGAAAAAAACAGCTTCCCAAATTGCAGC
CACCCTGCCTAGTTCAGAGACTGTAGAATTTGTATTCCTCCCCGAGTGTGTTCTTAGTAGCACGCCAGAAGTGCCAGATGCTACCCT
CTCTTTTTCTGTGTCATGATTTAATACCATTTGTGCATTTTCTTCTGCTTCAGTTGATTTAGTGTCATTTTTGGAGACCACAGACCT
ATTTGTATTTTATATGTTACTATACAGTGTGTATTCTGTTTAGAAAAATAAAAACGCTAGTATGATAAAATGTACTATTTTATTATC
TTTATTTTTAAAGTGTTACTATCAAAGACATCCTTTGGTGTCTCATAATTGGCTTTGACTTTGTTGCTCTCAATAAGAGTATATGAT
TCCAGATTTTCTGTTTAGTCAATGTTTAAACTACTTCATGTTTAACATTTATGCACATTATAATACAATGTGGTAATAAGGGTCATT
TGGCTTCTTGGCTCTTTCAAAATATTGGTATAATTTTTAACAAATTTATTTTGAAATAATCATTTCACTTTTTGATGAGTATAAACT
TGGTGAGAATTAGAAACTTCTTCAAAATATAGAAGTCTATTTTCCTTATCTTTGGCAAAGAATGATAATAATTGGACTTCAAAAGAC
CCAGCCCAGTAGTCCCCACTGCTAAAAATGAATAATGGCCTATCTCAATCCACAGAAGGAAAATAAAATGGGCTAATAGAAACTTTT
TCAGTTTCATGTTTTTCTCCTGTCAACATTTCATAAGTGTTTGGCATTTGCTACCACATCTCTTTTGGTGAGTATTAGTGAAAATGA
ATGAATAAGTAGTAAAAGAATGTTAACAGAAAAATTTGGACACAACCAGTTAGTTTAATAACTCTATTATTTTATTGAGAACACGCA
AGTTTAGGGTATGGTGTTTTTTTATTCTTTCTTAGGGTTGAAATAATCTCCCCTGCCAGGTACGTATAAGCTATGGTTTTAGATGGC
ATTCATATACTGCCATTTCTGCCAGAAAAATTATTCCTCGTTCATAAAAAGAAGCAAAGTATCTCAGTGTTTCATATTTTTCCTCAA
TAGGCAGCTCTGCTTCCCAGTGCTTTATCATCAGAGCAAGTTTGAAACCCACCACGTGGGTGAATGAGATGCAGTTAGGGAGTCTCT
GCGAGCTTCCTGAGACAGGAAAGCATGGGTCATCCTAAAGTTGTATAACAATCATCTGTGTATCCTTCTACAGGAAGGATGGAATTG
ACTTTTCCACCCACAGATTTGGCATAAAATGTCTCTGCATCTGGGGCCAAGACAGTGTGTAAGGATTGTGAAGTCCCAGTGGTTTGG
GTAGTAGGTGAAGATGCCAGTTCAGTGCTGAAGAGCCATTCCTACCTGTGTGTGTGTGTGTGTGTGTGTGGGGGTGCTGTGTGTGTG
GGGGTGTGTGCTGTGCACGTGTGTGTGTGCGCACGCACGTGTGTGTGCGCGCGCGTGTGTGGAGTGATTTTTCACTCTTTTTGCAGG
GAGGAGAAAAGAATGAATTAGTAGCTTTGCTTTTGCTAAGGATATCCTAACAGTGTGGTGGATCCTTTTTGTTTAACAGGGTAGGTC
AGATTACTGAGGAGCTGTTGGTTTATTGTTTTGTCATTAGTGCCATGAAGCTGCTACCGCTGGTTGTGGGTAAAGCAAGGCACATGA
AAGCAGTCTGAAAATTTGTATTCAAGCTAATTTTTAATTTTTAACTACTTGTTTACTTATTTCTATATTTACCCTTTTAGCATAATA
CCTGAAAACTTAAGGGGTTGGGAAATATCATTCATACAGCCAATTTTTTTTAATACCTTCTGTATTCAAGGCACTGTGCTGGGGAAA
CACAAGTGGATAAGATGGACATGGTCTCCATTCTCATGATGCATACAGCCTACTAGGGGAGACAGCTGAAAAACAACTACAGATATA
ATTAGAAGGAATAATTTCAGGAAGTGTGGTTAGTAATATGAGGGGGGAATAAATTCGCAAAGGTGGCCAGAAAAGACTTTACTGAGT
TATCATCTGAAACAAACCCTTAGGGTAGGAGTACCCTGTCTTTATAAAGGAGAGAAGAAAGAGATGCAATTAGCAGGATGATTGACG
TGAAAAATTGCTTGATATAGTCAAGGAATAGAAAGTAGGCCTGTGTGACTGGAGAGAGTGGTATGATGTGAAATTGGAGAGTTGGTA
GAAAGGAGAGCACAGAGATCCTTAGGGATTATGAAGTATCGTATGGTGCACATACTGATGGATTTTCACAAACTGAAGAAACTCTGT
AACCAGCACCCAGATCACGAAGTTGGACATGTTCAGTACCATTGGGGCTGCTGGAAGCAGTAACCACCCACGCTGTTAGCTCTCTTG
TGTTCACATTTCAGGGCCTTGCTTTAGGAAAACTTCTGGTGTTCTCAGGCTATTTCTTCTATTACCATCCAAGGCCTAACACTTCTA
GCTCCAAGCAGAGTATCTTTCTGTGGGATTTTTATACTCATGTCACTTCCTCTCCCCAACCCCCACCTTGAACATGTATTATACATG
GAATTCTTTGGTAAATAGAATGCAAATTTGTTTTCAAAGCTTGTTTGGCACATACTCTCTCCTTTAGAAAGCAAAGGGTGTCCTTTC
AGAAGGTTCTTATAATATTTGTGGATCTGGCCTTCAGGTCTAAGCTGAAGCTTTTGTTTTCATTTGGACTTTATGCATGTATTAACA
GTTTATATTTTCTGTGGTAATGGAATATTTTAGGGGTTGTTTCTTCATTATATTTAGATTCTTATATTAAAAAGCGTGCAAAGGCAT
TTAACCAAATTTAATCTAGAAATAAAGATAATATGGTAATTTCAAATAGCTTTTTGTTCAAGATGAGATTTTAAAAGTTATTATTCC
ACAGTGAATTTTAATGATGATTGTGTACAATGGCATTTATGGATGTGCAGCAATTGAATGAGTATGATTAGCGCGTTAGATTATGGA
TTTGTAATGTGAAATGAGAACTTTTTAATAAGCTTTCTCCATAATGGTTCACGCTGTGAACAAACAAAGTATGAGACACTGAGCTTC
ACTAAATCATTGCAGGGCTTTGGAATCAGTAGGTTCATGTTTTCAAATAGTTTCCTATTTTTAAAAACACCGCTAAAAAGTTAAGAG
GCAGCTACATAACTAGATATCTTATATTTTGAGGAGTTGGAAGAACATGTTTCTTTTTATAATAAAAGGAAAATGTTATTGTTGAAG
TATATTTATTGTTCAAGTATAAAATGGAATTGAAGTTATGCTTTGAGAAAAACTTGATTGCAAGACCTTATTAGGATTTAACACCTC
ATCAAGACTGCTTTTTACATTTTTTTTTTTATTTATTTTTTTTTTAGATAGAGTCTCGCTCTGTCACCCAGGATGGAGCGCAATGGC
ATGATCTCGGCTCACTGCAACTTCCGCCTCCCCAGTTCAAGCGATTCTCCTGCCTCAGCCTCCTGAGTAGCTGGGATTACAGGCGCC
TGCCATCACGCCCAGCTAATTTTTGTATTTTTAGTAGAGACAGGGTTTCGTCATGTGGGCCAGGCTGGTCTCGAACTTCTGACCTCA
AGTGATCCACTTGCCTCAGCCTCCTAAAGTGCTAGGATTACACGCGTGAGCCACCGTGCCCAGCTGCTTTTGACATTTTCTTTGGGA
GGTTTCTGGGGTACAATTAGCTGAAATCTGAAGTTTGGTTTTGCATGGTCTTGCCCAAGCTGGTGGTTGGTCATTGTGGCCTTCTGA
TCTGGTGATCCTAAGAAACATTCTGAAAACATACCCAAAAACACATAAAAGGCGTACCTTACCATTTAAAGGTTCTTTCAGTAAAGG
CCTATGAGTAATTTTTCATGGAGGGAGCCCATTCTTACGAAGTTTTAATGGACAAACATATCATTATCTTTAAATTGTATTATTACG
TTGTCTTTTATTTTTAAAAGCTCATTTTTCCCCATTATCAAGATAACATGTTCTAGACAGACAATTTAGGAAATGCATATGATTATT
TTTGTTCTAATTTTCTGTAATACCACCACATAGAGACAGTAGTTTTTAAAAACTGTGTGATATTTAATGTATTGTTTTCATTTCCTT
TAAGATGCAATTTAGAGTTTTAGCCTTGAACTTTAGTTTTCCTTCCTCTTGGAGATTTAGTTTGATGCTAAAGTGCTATCTTCTTGT
TTCAACTTTTGCTTAATTTACTCCAACACTTTTAGACAGCAAATTAGGTAACTTTATACTAGTGTTATCTAACAATGTTTTGCTAAC
TTCCATTTTAAGATGTTAGCTCACAAAAAAAGCTCTTAATTTAAATACATATAAAATTTTGAAAATGTGTGACTTTAATTAAAGACC
ATTGTTGGCTAGCATCATGGAGGTGTCAAGAAAATTAATAGTAAATGGGCCAGTCATCATTTCAAAACTTTCATGTTACATGACAAC
TGAAATTCCAGATACCCTTTAAGAGGTAAACTAGTTATGGAGAAGGGATGGAGAAGCACAGAATCCTAAATTTACTGTGGTTAGTCA
TGCACTCTGTGAGATGGAGGTTATTAGCCTCATATACCTAAAAATATATCCAGTGACCAAACCATGTCATAGGGCTCTGTCCATTTC
TTGCCTTTGCTTTCTCTTGGACTGGCTTCATTCTTAGGGTATCCTCATGTGGTTTCAAGGTAGCTACTAGCAGCTCCTATTGTCTTT
CCCATGAGTTTAATAACTGTAGTAAATGAGTCTCTCTTTGAAAATACTTGAATAGAAATCCCAGGATGAGTCTTACTGGACAAGTCT
TAAGCACCCACTAGTCCCAAACCAATTGTTAATGTGAGATAGGTGGAATGCTGTGATTGCCAGGATTGGGTCACATCCTTACCATCT
TACTCTAGAACACCAGCTTATTGCCCAAAGAAAGACCAAGGTGTTGTAATTGGAAAAAGGGAGGCAGACTACAAGCACACAGGAAAA
CTTCCTGGGTGTTCACTAAAAAGGTCGATGAAGAATGAAGCATAATTCCTTGAATGAAAATGAAAATACAGCATAGTGAAACCTGTG
AGATACTGCAAAAGCAGTACTAAGAGGGAAGTTTATAGCACTAAATGCCTACATCAAAACAGTAGGTCACAAGTTACCTAACATCAC
ACTTGAAGGAACTAGAAAAATAAGAACAAACCAAACTCAAAGTTACAAGAAAATAAGCGACAAAGATCAGAGCAAAACTACATGAAC
TAGAGACCAAAAAACAATACAAAGGATGAGTGAAATGAAAAGTTGGTTTTTTGAAAAGATAAACAAAATTGATAAACCACTAGCTAG
ACTAACCAAGAAAAGAAGAGAGAAGATCAAAATAAACACAATCAGAAATGAAAAAGGAGACATTACAACTGATCATGCAAATATAAG
ATCATCAGAGACTATTATTATTAACAACTATATGCTCACAAACTAGAAAAGCTGGAAGAAATGGATAAACTCCTGGGGACACAACCT
CCTGAGATTGAAGAAATAGAAGCCCTGCACAGACCAATGATGAGTAGTGAAATTGAATCAGTAATAAAAATCTCTCAACAAAAAAAG
CCCAGGACCAGATGCAGTCACAGGCAAATTCTTTCAAACATACAAAGGAGGCCAGGTGCAGTGGCTCATGCCTGTAATCCCAGCACT
TTGGGAGGCCGTGGTGTGTGGATCACGAGCTCAGGAGATCGAGACTATCCTGGCTAACACTGTGAAACCCCATCTCTACTAAAAATA
CAAAAAAAAAAAAAATTAGCTGGGCGTGATGGTACCCACCTGTAGTCCTAGCTACTTGGGAGGCTGAGGCAGGAGAATCCCTTGAAC
CCGGGAGGCGGAGGTTGCAGTGAGCCAAGATCGCGCCACTGCACTCCAGCCTGGGTGACAGAGCAAGACTCCATCTCAAAAAAAAAA
AAAAAGAAAAGAAAAAGGAAAAAACCATACAAAGAAGAACTAATACCAATCCTCCTGAAACTATTCCAAAAAAAAAAAAACGAGAGG
ATTCTCCCCACCTCATTCTACAAGGCTAGTTATAACCCTGATACCAAAACCAGACAAGGATACAACAAAAAAGAAAACTAGAGGCCA
ATATCCCTGATGAAAATATATGTAAAAATTCTCAACAAAATACTAGCAAATTGAATCCAGCAGCTCATCAAAAACATACTATACCGT
GATCAGGTGGGATTTATCCTAAGGATGCAGGATGATTCAACATGTGCAAATGAAGAAATGTGATACATCATGCAGACAGAATTAAAG
GACAAAAACATATGATCGTCTCAATAGATGCAGAAAAAAGCATTCAGTAAAATTCAGCATCTCTTCATGATAAAAGTCTTCAACATA
CGAGACACAGAAGGAATATACTTCAACATAATAAAGGCCATATATGACAAACCTACAGCCAACATCATACTTAATAGGAAAAAGTTG
AAAACATTCCCCTTAAGAACTGGAACAAGAGAAGCATGCCCACTTTCATGACTCTTATTCAACATGGCACTGGAAGTTCTCACCAGA
GCAATCAGGCAAGAGAGAATAATAAAAGACATCCAAATTGGAAAGGAGGAAGTCAAATTATCCCTGTTCACTGATGATATGATCTTA
TATCTAGAAAACCTTAAAGACTCCACCAAAAAACTCTTAGATTTGATAAATCAATTCAGTAAGGTTTCAGGATACTAAATTAACATA
TAGAAATCAGTAGCTTTTCTGTACACTAGTACAGTAATGATCTAGCCGAGGACCAAATCAAAAAGTCAACCCCATTTATAATAGCTA
CCAAAAAAAAAAAAAAAAACCCAGCTGGGAATATATTTAAACAAGGAGGTGAACTACAAAACACCGATGAAAGTAATCATAGACAAC
ACAGACAAATGGAAAAATATCCCATGCTCATGGGTTAGAAGAATCAACATTGTTAAAATGACCATCTCCCCAGAGAAATCTACAGAT
TCAATATAATCCTTATCAAACTTACCAACATTATTTTTCACAGAATTAAAAAACCCAATCCTAAAATTCATATGGAACCAAAAAAGC
CAGAATAGCCAAAGCAATCCTAAGCAAAAAGAAAAAATCTAGAGGCGTCACGTTACCTGACTTGAAATTACACTACAAAGCTATAGT
AACCAACAACATGGTACTGGTATAAAATAGACACATAGATCAGTGGAATAGAATAGAGAAGCCAAAAATAAAGCCACATATCTAAAA
CAACTGATTTTCAACAAATTCAACAAAAATGTACACTGGGGAAATGACACCCTATTCAATAAATGGTGCTGGGAAAATTGGATGTAT
GTTGTAGAAGAATGAAACTGAACCCATACCTCTTATCATATACAAAAATTAACTCAGGATGGATTAAAGACCTAAACGTAAGACCTG
AAACTATAAAAATCCTAGAAGAAACCTAGGAAAAACTCTTCTGGACATTGGCCCAGGCAAAGAATTTATGACCAAGTCCTTAAAACC
AAGCACAATGAAAATAAAAATGGAGAAATGGCACTTAATTAAATTAAATTAAAAAGATTCTGCACAGCAAAAGAAAAAAATCAGGAG
AGTAAACCTACAGAATGAGGAAAAAAAAATTCCCAAAGTATGCATCCAACTAAGGGCTGACATGCAGAATCTACAAGAAACTCAAAC
AACTTAACAAGAAAAAAACCCCAAAGAACCCCATTAAAAAGTGAGCAAAAAACATGAACACACATTTTTCAAAAGAAGACATACAAG
TGGCCAACAAACATGAAAAAAATGCTCAACATCACTAATCATCAGAGAAATGCAAATTAAAATCACAACGAGGCCAGGTGCAGTGGC
TCATACCTGTAACCCCAGTACTTTGGGAGACTGAGGTGGGCAGATCACAAGGTCAGGAGTTTGAGACCAGTCTGGCCAATATGGTGA
AACCCTGTCTCTACTAAAAATACAAAAAACACTAGCTGGGCGTAGGGCACCTGTAGTCCCAGTTACTCAGGAGGCTGAGGCAGGAGA
ATTGCTTAAACCCGAGAGGCAGAGGTTGCAGTGAGCCGAAATCACACCACTGCACTCCATCCTGGGTGACAGAGTGAGACTCTGCCT
CAAAAAAAAAAAAAAAAACAAAAACAAACAAACAAACAAAAATAAAACAACCACAATGAGATACCTTCTCACACCAGTCAGAATGGT
TGTTATTCAACAGACTAAAAATAACAGATGTTGGTGAGCATGTGGAGAAAAGGGAACGCTTATACACTGTTGGTGGGAATATACGTT
AGTACAACCTCAGTGGAAAATGGTATGGAGATTTCTCAACTAAAAATAGAACTACCGTTGGATCTAGCAATCCCATTTCCGGTATCT
AACCAAAGGAAAAGGAATTATTATATTTAAAAACACCTCAAGTCATATGTTCACCACACTGCTGTTCACAATAGCAAAGATATGAAA
TCAACCTAAGTGTCCATCAGTGGAGGATCATTGGATACAGAAAATGTGGGTTACACACACACACACACACACACACACACACACACC
TTGGAATACTATGCAGCGATAAAAAGCAATGAAATCTTGTCTTTTGGAGCAACATGGATGGAACTGGAGGCCTTTATCATCAGTAAA
ATAACTCAGAAACAGAAAGGCAAATGCCACACATTCTCACTTGTAAACGGGGGCCAAATGATGGGTACACATGGACAGACAGAATAA
AATAATGGACATTGAAGACTAAAAATAGTGGGAGGATGGGAGGGAGGTGAGGGTTGAAAAATTATGATTGGGTAGAAAGTTTACTAT
TTAGGTGATGGGTACATTAAAAGCCCAGACTTTACCACTAGGCAATATATGCATGTAAGAAATCTGCACTTGAACTCCCTACATGTA
TTTTTAAATTTTAAAAATTAAAAAAAAAATTGCCACTGTGCCATTAGCTGGTACCATGTAATTTAGCATATAACTGTGTACGGATCT
CTTTTCTCCATCATTGCTTCATCACGTTAATTTTGTTATGATGAGGGACTGTCTTTTACTTTTTTTATATTCCTCTTAGCACTAAGC
AAATAACAGGACCCTAAACACTTAACTGATTTTTCATTTCTGAAGTACAATAAAGATAAGTAGGAAATGTTAATATCATTGTTTTGT
TTGTTTGCTGGTTTTGTTTTTCAGTAGGTGTTTGAGAGAAACACTTAACTCTCTAAGTGAACTAATTTTTTGCCATGAATGTGTTCG
GCACTAGGAAAGGTGATACATACTAGAACTGAGGAACTTAACTGGGGGTTCATATGCTTTCTAAGAGACCGTGGGAGAGTTTCTGGG
CATCTCTGCACCCCTAAAATCCTAGCAAAATTGTGTATGTGTGCACGCTTTTGTGTTTTTTTCTTTTTCTTTTTCTTTTTTTTTTTT
TTTTGAGACAGAGTCTCGCTCTGTCGCCCAGGCTGGAGTGTAGTGGCATGATCTCGGCTGTCAGCTCACTGCAGCCTCTACCTCCAG
GGTTCAAGGGATTCTCCTGCCTCAGCCTCCTGAGTAGCTGGGATTACAGGCACCCACCACCACGCCAGCGAATTTTTATATTTTAGT
AGAGACGGGGTTTCACCGTGTTGGCCAGGCTGGTCTCAAACCCCTGGACTCAAGTGATCCGTCTACCTCGGCTTCCCAAAGTGCTGG
GATTACAGGCGTGAGCCACCACACCTGGCTTCTTTGTGCATTTTTCCTGAGTAAGGATGTGTAGCTTTCATTAGATGCTCAAAGGAG
ATCCTTAATCCACAGAAGGTTCAGAGCCACTGATAGGAAATATTTAACACTGACAGAAAATAGTTCTGAACTTTGCGATTAAATAAT
TTATTGTGAGTGGGAGGAGCTAAAAGAGTGATGCCACTTAGGAGAAGCACAAAGGAATCTGAGCTCGAGGATGAATTGTTAATCTAT
GTTAATGAATTCAGTCTGTACAGGGTTCAAACAAAATATATTATGGATGAGCCTCTAATAAAATGGACTTCCTCTGCTCTGCCAGTC
CGACAGTTAATGATGGCTGGCAATTTTCTGCCGGCTGATTTTGGCTCAGTGCTTTGGAGCAGGCACAGGCCCAGTTCTCGCCACTTG
GAAGGAAAGGGAAAGTGATAGTTTGTTACTTTGCTCTCATAAGCAATTTATGACAACAGTTTAATAAAAGCTTAAGAAGTGTCCTCT
TCCTGGGCGGGGAGAGGAGCCGAGGAGCCTCAGTGGGAGCACCGCGTGGGATGTTAAGCCCCTCAGCATCTCCTGGGGCCGCCTGAC
GCTGCCACACAAGAGAAGAGCGAGGGGGAGCCAGCGAGGTGTTTCTTTTAGCCCAGGCAAGGCTACTGTTATGCTTCCCAATTTGAC
TTTTCCCCAGCAAACTCAAGCTGTCCTGTGAGTTTCCACATCAGCCGTGTGCCTCGGGAGCTGCTCCGAGCTTGGGCTCTGGTGCTG
TCTGCTGCATGGACCTCATTAGCGCTCACCCAGCAGATTGCTTTCATATCGCTGCTTCACCCTTTTCAGTTTTGAATTACCACATAA
ACATCAGATATTGGGGGTTTTGTGAAAGGATCCTTAGTCTCTAATATTTAGTTAAGGAAAAAAGCCCTCTTAGGGAATCAGATAGAT
TTGCTTAGAGGATTTCCTCAGGAAAATACATTGCAGTATGGGTCAGTTCTAATTTTCCTCAATTCCTGAGGCCATTTAGTTTTCACT
TCTCTCTCTCTCTCTCCCCACCTCAACCCTGTGAGTCCAGGTTTAGGGGATTTTACAGCCAGCCTTCACACAACTTTCTGCTCTACA
TTTGTGGCCTTAAAACTTTATGATTTATAGGAAAGTATATTTCATGTCATGAACTGGGATATAAATATGTATGTATATATGTATAAG
CAAAAAGGTTTCACAAGGCAATATTTGCCCTTATATACCATAGTCTGATATTTTCTATTCTGTCGGGGAATACTGCTGGTCTTCACT
CATTAAATCATTTTCATGGCTCACCAATGGTTTGCACCCTGCAGTTGGAAAACACCTCTCAGGGATACTCAGGGATCCGTCCTTTCT
ATCACTGTGTTGAGGGATGGCTCAGGGCACCGTGTAGTGGCTGAACCTCTTTATGTGGTACAGCAGAACCAACTTTATGTATTTTCA
GTAACACTTCTGATATTCTGGAGGGAGTAGAGCATAGTGATTTAAGAGCGAATGTTCTAGTTAGAATATGCTTGCTTGGTCCTGGGT
CTCTGACCTACCACCTGCTCTGGTGTTGGGCAAGTTTCTTAACCTCTCTGAATCAGTTTGCCCTTCTTTAAAATAGATATAATTTGA
ATACTTATAGCGAAACATTGTGAAGATTGAGATGTATATTATTAATACATTATTAATATATCCTAATGCACATCTATTAAGACACAA
TACAGAATTAATATTAATAGCAAACATTTGTTCAATGTTACTATGTGTCTGTGGTAAATTTAACAAATGTTTGCTATTAACATTATT
AATACTGTATCTTAATATTTATGTGTATCTCCAGTTGGATTACATTTTCCTTCTGCCCTTTGAAATAACTTATGTTGAGCCTATTAA
GAATATGTGGGTTGGGTGTGCTGGCTCACGCCTGTAATCCCAGCACTTTGGGAGGTTGAGGTGGGCATATCGCTTGAGGCCAGGAGT
TCTAGACCAGCCTAGGCAACATGGTGAAACCCTGCCTCTACAAAAAATACAAAGAAATTAGCCAGGTGTGGTGGCACACGCCAGTAG
TCACAGCTACTTGAGAGGCTGAGGTGGGAGGATCACCTGAGCCCAGGAAGCAGAGGTTGTAATGAGCCGATATCACACCACTGGACT
CCAGCCTGGGTGACAGAGCAAGACCCTGTCTCAAAAAAAAAAAAGTCTTATGTGTAGAAATAATCCCTATGTTCAAAAGCATGAACA
GTAAGCAGTGTGGTTAAATGAAAAAATATATATATTGCAGCTAGATAGTTACATTTTGAATTAGGACATTTTCAGCTCTAAGTGACA
GAAACCCCAATCAAACTGGCTCAGGCAAAAATAGGAATTTACTGATTGACGTAACAGAAACATCTAGGGATATTGCTTTTGGCATTG
GCCATTTCCAGTTGCTCTGAAGGTATCAGCAGTTTTTCTCTTCATTTCTTGCTCTGCCTTTCTCTGTTGGCTTCTTTTTCAGGCAAG
CTTGCCCCCAAGCAGTGGCAGAAACAGCCACCAGCTGCTCTAGGTTTCTGTTTCACTAGCAGGAAGAAAAACCCTCTTCTCTGGTAA
TTTCAGCTTAGTCTTGCATCTGATGGACATGGACTTTGGTGGGCTAGGCTCAGCTCACCCCTCCTGACTCCTTGCCACTGTCGGTCA
CGGAGCCATGAGAGGCACTGGTCATTAGATGTCCTGGGTCATGTACTTGCCTTGTAGGCTCAGCCCTGTTCAAGCCACAAGGTCAGA
ATGGAGGAGGGCTGCTCCATGGGGGCATCTCAGCAGATGCCCATCACACACTTCCACCATCCCTCCCCTGAACATAAGACTTCCTAC
ATAGATGTTTTCTTTGAATAATTCAACTGAGAGTAGTGTTAACACCTAGATACTTCTTATTCCAAGATATACCTCATTTCCACTCTT
AATTTAAATTATAGCTATGTTCAAAGATGTGTCATCCCTTCCTTCCTGATCACTGCTAAGATGTTCTTTTTAGCCACCCCGTCTTTG
TATTGCCACTTAAAAGTCATCATTTCCTTCTATCTCATTTTATGCCTCCCTCTGCTTTCGCACACCTTTAGTACTTGTTGCCGTCGT
TATGAACAGCAAAATACAGACATGTGACACCCCACCTCCTCAGCAGTCTATCCTTTTCATTTTGTGCTCTAGCCAGCCCAGGGGTCA
GAACTGCATTCTGCAGCTTTTCTTTCATTCTCTGGCTGTGAATGAGGGTCCCTGATTTACCAGACTACATGAGGTTGGGACTGTGAG
TTGTTGGACAGGCCATGGCTTCCCCTATACACACACACACACAGACACACAGATGAAATGGTTGACAGTGGACAGTTTGGTTGCAGT
AGGGACTCCAGATGGTGAAGTGTGTCTGCCAGGGAGTTAAAGAGAATTGTGGGTCTGGAGCCAGTGAAATTGAGGGCACATTCCAGA
GCTTACGTCTGCCATGTGCAAGATATGTTTAGCTTGCTAATTAAATTAGGAACAAGGTAAGCACTTGTTTGTTCATTCATTCATTCA
TTCATTCAGAGACAGGGTCTTGCTCTGTCACTCAGGCTGGAGTGCAGTGGTGTGATCATAGCTCACTGCAGCCCTGAACTCCTGGGC
TTAAGGAATCCTTCTGCCTCAGCCTCCCGAGTAGCTAGGACTACAGGCGTGCACTACCACACTTGGCTAATTTTTAAAAATTTCTTG
TAGAGACAAGATCTCACTATGTTGCCCACTTTGGTCTCGAACTCCTGGCCCCAGACAATCCTCCTGCCTCGGCCTCCCAAAGTATTT
GGGATTACAGGTGTGTGCCACTGCACCTAGCTGACATTTTTTAAGCCCAATATTGTGATCTAATATTCTATAACTGTATAGGATGGA
TAAATCAAATGTCGTGAAGTTCACATATAGAAAAGGTATGGATTCAGGTAGTCTCAAGGTATGCATTATTTGGGGGAAAATTAGAAA
AATTCCCTAAAGACATTTAAAAATTCTAAAGTAGGCTGGGTGCAGTGGCTCACACCTGTAATCCCAGCACTTTGGGAGGCCAAGGCA
GGTAGATCACTTGAGGTCAGGAGTTCAAGACCAACCTGGCCAACATGGTGAAACCCTGTCTCTGCTAAAAATACAAAAAAAAATTAG
CCTGGCGTGGTGTCGGGAGCCTGTAATCCCAGCTACTTGGGAGGCTGAGGCAGGAGAATCGCTTCAACCAGGGAGGCGGAGGTTGTG
GTGAGCCGACATCACGCCACTGCACTCTAGTCTGGGCGACAGAGGGAGACTTCATCTTTTTTTTTTTTTTTTTTCTAAAGTAGCAGA
ATTGTTATGTATGTATTTATGGATGGGTGTATATATATGTATATTAAGAAATGCTAGTCAAAGTGCAGATATGATTTACACATTATC
TAATGGAAGCTTTCTAACCCCCGACCCCATAGGCACATCCACGATGCGAGAACACAGGCTCATGCAGAGTTGAAACGCTGTTGCCAA
ACTAAATCAAGACTGATAATTGGCCAGTTAGCTCAAAGAGTCAATGAAAATGATGAGGGTGTGTGTAAAATGACACATACTTTACAC
ATTTTATTTTAAATGAAAATACGATTTTGAAGTAGTATATTGTCTAGCATTTTGGTTTTTTCCCTGCTCGTGCTGTGTGCATGGAGA
CTCTGATATTTTTCCCCCAGTGGTATGTCACTTGCTGTAATATTTTGAGTACTAGGAATGTTTTGAGATAGGCCCAAGGTCTTTTTC
TTGTCTAAGTGTCCTCTGACTGAAGTATCTTCCTTGTGTAAACTTCAGCCATAATTTGTCAGCCACAACTTCCATTTACATTTTTTT
AGGTGGACCTAGAACTCAGCAATACTTGCAAAGTAATCTAAGTCGGGAGGTTTTTCCAGAATTTTTCCAGTGTTATGCAAGTTGTCT
CCGTCACACCTCATTCAACAGCAAACTTTTGTCATGGTTTGCCTTCATGGAGTCTTTTTTTTTTTTTTTTTTTTTTAAAGAGATGAG
GTCTCACTGTCACCCAGGCTAAAGTGCAGTGGCACGATCATAGCTAATTGTAACCTTGAGCTCCTGGGCTCAAACGATCCTCCTGCC
TCAGTCTTCTGAGTAGCTAGGACTATAGGCATGTACCACTGTGCCTGGCGAATTTTTAAGTCTTTTTGTAGAGAATGGGTGTCTTGC
TGTTTTACCTCCGCTGGTCTTGAACTCCTGGCCTTAAGCGATCCTGCCACCTCTGCCTCCCAAAGTGCTGGGATGACAGGCATGAGC
CACTATGCCCAGCCTGCCTTCATGGAGTCTTTACAAGGCATTTAGCTTAGTTTTTACAGAAACAACTATTTCTTGACTTCCATATTT
AATTGATTTTATATAATGCTTGCACAGTATGCATGCAAAGCAAGTACACTTGGCACAAAAGATTTGAAAGGAAACATAGCTCAACCT
GGAATAAGCACACAGCTCCATGGGTGGAACGGATGTGAGCAGATGTGTTGGCACTAGGACCACTGGTAGCTCACACTTAGGTGGGGC
TGTGTGTTCTAAGTTCTTTGTATACATTGACTCATTTCATCTTCACAGAACCCCGTCAGGTAAGATATTATTATCCCTTTACACAGA
TGAGGGAAGTTATGTACAGAGAGGACAAGAGATTTGCTTCAGAGTGGCCTACTAGCTGCCAGCATTCAGCCTGCATTGGGCTTTAGA
CAATATGTTTTGCGAAGGAATGAGAGCATTTTGTGTGGGGGTAAATTAAGAGCACAGAGACACTATATAGAGATAATCCCTTAAATA
AAACCCACCATAACACATAAAGACTTCTGAAATACTCTCCATATCTCTCTAAAATTAGAGTCTAGGCTTTTAGAGGTAGAGATGACC
TTAACGTATCCCCTGCATATCCCAACAGGATAACCAAGACCCAGATGGGTGAGGTGATTTGCTTCACAGGCACCCAAGAAAGTGTGG
CATAAAGTTTAGTTTCCTGATTTAACCCCCATTTTCCTTCTCTCTCTCTCTCTCTCTTTTTTTTTTTTTTTTTTTTTGAGACAGAGT
CTTGCTCTGTCACCCAGGCTGGAGTGTAGTGGCATGATCTCGGCTCACTGCACCCTCCCCCTCCTGGGTTCAAGCAATTCTCATGCC
TCAGCCTCCTGAGTAGCTGGGATTATAGGCATGCCACCATGCCCAGCTAATTTCTTTGTATTTTTAGTACAGATGGGGTTTTTCCAT
GTTGCCCAGGCTAGTCTCGATCTCCTGGCCTCAAGTGATCTGCCTGCCTCAGCCTCCCAAAGTGCTGGAATTACAGGCGTGAGCCAC
CATGCCCAGCCTATCCCACATTTTCTTATGGAAGAAAAGGATGCATTGTAGTGTATATGTAATCTTCTGAGAGAAACATTCAGGAAA
TGAGCCTCTCAGAGTTTGCCAAGAGAGCAGGAAGGTTGGTGGATAAGGACCACCTTAGTGTTATCAAATCCACATCTCTCCTGCAGT
CAGAGTAAAGCCTGTACCAGGTAATCTTAGGACTAGAAAGGGTCTAGCGATGTGGTTTTATCCACCCCTGTCTAGAGAAGGAGGGAA
TCAGGCCAGCTGAGCACCAACAAGGTAGATGGTTTTGTCACTATTTTATGGGTAAGGAACTTGAGCTTCAAAGAGAGAAAATGATTT
ATCCAAAATCACACTGCTAGAAGGAAATGGAACCAGGACACAAGCCTGGTCTTGCTGAAGCTAAGGCCCGTGTTCTTTATGCCGTGC
TAATTGTTCATCCTGTGCAGTTGTTTAACCACAGTTTTCTTGGCTAGAAGTTCTTCCTAAAGTTAACCCTAAATATCTCTTGAATTT
AAATGTGTATCCTTCTGTAGTCCTCAGTCAACAAAAGAAGCGATTGATCAGCCTATTTGAGAGGTTATAAGAAGACACAGATTGGCA
GTGTTTGAATGTCAGTAGTACTCATCAAATAATCCCAGGATAGAAGGCATTTTGTTCAACTCCCCATTATCCCTGCTCACATTTGCT
CTGTAGCATCCCCACGAAGCCTGTGCTGGGTAGTTTTCAGTCCGGGAATTAAGAGATATAAGAAAGAGGGGGCTGGGCACGGTGGCT
CACGTCTGTAATCCCAGGACTTTGGGAGGCTGAAGCAGGCAGATCACCTGAGGTCAGGAGTTCAAGACCAGCCTGGCCAACATGGCG
AAACCCCATCTCTACTAAAAGTACAAAAATTAGCTGGGCATGGTGGCACGTGCCTGTAATCCCAGCCACTCAGGAGACTGAGACAGG
AGAATAGCTTGAACCTGGGAGGCGGAGGTTGCAGTGAGCCGAGATTGCGCCACTGCACTCCAGCCTGGGCTACAACAGTGAGACTCC
ATCTCAAAAAAAAAAAGGAAGAGGGAAGTCACCACCTTAGCCCATCCCATCTTCTTCCCAAGAGGTCATACACACATATGCCGTCTT
TGATTTGTATTTGCAAAAGTAATTCATGTTTATAGTAGAAACTTGGATCACTGAAGAAAGCTCAAGAAAGAATTTGAAAATCTATGT
GTATGAGTTATCTATTGCTATATAACAAGTTACCCCAGAATTTAGCAGCTTAAAACAAACTTTTATCTCCCATAGTTTGAGAGTCAT
TAGTCTGGGAGTGGCTTAGCTGGGTAGTTCTCTCATGGAGTTGCAGTCAAGCTGTGAGTTGAGGCTCTAGTCACCTGGAGCTTTGTC
TGGGGCTGCAGGGTCTACTTTCAAACTCACGTGAGTGTTGGCAGGAGACTTCAGTTCTTGCCATATGGGCCGCCTCATGGAGCTGCC
CACAGCATGGCTTCCCACAGAGCCATGTTGATGAGAGAGAGAGAGAACACGCACTAGAGAGCAACCAAGACACAGCCACAATCTTTT
CTACGCTGATCTTAGAAAGGATGTTCCTGCACTTCTGTTGTATTCTGTTGGTCACACAACCAACCCCGGTATAATATTATAGGAGGG
GACTACACAAAGGTGTGAGGACCAGGGATGGGAATCATTGAAGGCCATATCGAAGGTTGCCTACCACAGCATGTAAATTAAAATTAT
GTTGCAATAAATTACATAAGTTTTTGCCCACAGTTCTCCTTTTCTTCCCCCTAGAGTATTGCAAGATACAAAAGATCACCAGGAAAA
AAAAAATCTAAAGGGAATGCAATGTTGAGGCATGAATGTCCCAGTAAGCTGTTTATTCCTTTCTCATTCTAGAGGAGCTTCTTGATT
TTATTCCAAGTCTGCCATGTCAAAATTGTGGTTTGGGGAAGAGGGGAGTAAGGGCAATAGAGTGGATAGAAGCATGTGGATGCGCCT
TGGTCCTGACATTTGTGTTTTGGTGAACCTGCTGCCTCTGAGAAAAGATAAGGAGTTTAGGATTTGGTCCAGGCTGAAACCTGGGAG
AAGCTCATGGCTCCTGGACCTCCTTATTCTGCTTTTAGATGCAAATAAGAAACAGAGATTTGTAAATGATATTCTAATTTTTACAAA
TAATTATTTGAGGAAAAAGATTTGACTACTTCAAACATACAAGCATTGAAAGTAACATAAAAGGTTTTTTGGTTTTTTTTTTTTTTG
GTGGGGGTGAGGGGGAGAAGCATTTTGTTTATGCAAATACAGGGTACAGAGATCCAGAAGGAGATCATAACATCTGGACAAGCAGTA
GCATTCCCCAGCCTCCCACCCCCAACTGTTACAGATAACAGAGGAAACTTGTGGGTAAATAAGTAGTAGGTAATACAGGCATTTATA
GATAGGGGACGTTTTCATATCAGACACGGCAGAGCTTTTTTTGAAAAGCACATACCTGTATGATATTTTGATGAATAAAGGTAATGC
AGAAGCAAACTGTAATATCAAATAAATTTGAGTGAATTCTTCTACAGTACCTACAGTCAAAGGAAATTTATCAGTGAAAATCAGTAT
ACAACAAACAGCAAAATGGAATAAATTTGAATAAATTCCTGCACAGGCAGTCACAGGACATGTTGATAAATATTTCAGTATAAACTG
ATACAAAGTATAGCATAAAATATTAATTTTGAAGGAATTCCTATGTCATGCCTTGAATAAAAGCTAATATTTCACTGAATAGTGTGT
ATGTACCAAGCAACGGGAAGAAAACTGAATTAGTTTTCACAAAATGGCTCTAACTTAATGTGTATAACCATTTTTCAGCTGACTTTG
AGCCACAGGTTGCAGTAGTCATATGGATGTGACTCTTAGATGTCAGAGGAATGGCCCTGCTCTGGTGGCGGGGTGGCTGTTGGCCTC
TGTCCCTGTCCATCCTGTGGACCTTTTTGTGGACTAAGGAGCTGATGTAGTGAACTGGTACTGCTGGTGTGTGGCTTTCTGGACCTG
CCTCCCTCAGCCAGAGGAGACCCTTGGATTTCTCCATCCTAAGCCAGACTTACTGATGGCCAAGAGTGAGGAGCAGGCAGAACAGCC
ACAAAGTAACTGCTTTGCTCTTTGGCTATGCCCTAAAGTGTCCAAAGTCATTGTTCTAAAGTGGAGCAACTACATAACTGACAGTTA
ACCTCTAGAGCCCTGTACAAATCCTTCCTTTTAGTCCAGATGCTATATGAGTTTGGGGAACCTGAGCCTTCCTGTGTGCACGTATTC
CCCTATTGCTGCTGATGCAGGGAAAACTGTGCCCAGTAGTGACACCATCCAGCACCTCAGGTGCCAAAGTCCCCAGGCCGTGATCAC
AGAGCATGGGGCTGAGCCCCTCATAGGTGGTCACATCACCCTCTTTGTCCTGCTGTCGGCTGGAATGTGTCCAGCCTAGCTCAGCAT
GGAAGAAGGTGAACCCCACCACCTACCATGAGCAGCACCTCAAATGACAATGCCACAGAGAAAATGGAGCAAAACCAGGATGACCTT
GCCAGCTCTTCATTGTAGGATCAATGCATTTAGGTGCAGAGGGGTGTGGATGGAAGCCATGGCCTAGCTTGGCTTGTCGCAGACCTC
TGTGCCAGGCCCTTGCCACCTCCTAATGCAGACAGCAAATACTGGAGGCCTGAGCATCAGCTCTTGAAGTAGCTCTCTGGGAGGGCG
GTGAGGCACATGTTGAGAGCATGGGATTTGCCAGCAGAAAGATCTTCAGTGAATTACCTAAGCTCATTACACCTTAGAGTCCTCATC
TGTAAAATGGGGCACTTGCCTAATGAGGGTGTTATGAAAATTAGCATATTTAAAGGTACTAGGGCTGTTTCTGGCACATTGTCAGGG
TTCAGTAATTCTAGTTAAAAATAGAAAAGTGGCTTCTGGCTGGCAAGATGGCCGAATAGGAACAGCTCCGGTCTGTAGCTCCCAGCG
AGATCGACTCAGAAGGCGGGTGATTTCTGCATCTCCAACTGAGGTGCCCGGTTCATCTCACTGGGACTGGTTGGACAGCGGGTGCAG
CCCACAGAGGGCAAGCCGAAGCAGGGTGGGGCGTCACCTCACCCGGGAAGCGCAAGGGGTCGCGGAATTTCGTCCTCTAGCCAAAGG
AAGCCGTGAGGGACTGAGCCTGAGGAACTCCGGCATAGATACTGCGCTTGTTCCACAGTCTTCGCAACCCACAAACCAGGAGATTCC
CTCCGGTGCCCACCCCACCACGGCCCTGGGTTTCAAGCACAAATCTGGGTGGCCATTTGGGCAGACGCCGAACTAGCTGCAGGAGTT
CTTTTTTTCCATACCCCAGTAGCACCTGGAACGCCAACGAGACAACCATTCACTCTCCTGGAAAGGGGTGCTGAAGCCAGGGATCCA
AGTGGTCTGGCTCGGCGGGTCCCACCCCCACAGAGCCCAGCAAACTAAGATCCACTGGCTTGAAATCCTCGCTGCCAGCACGGCAGC
AGTCTGAGCTCCACCTGGGGCACTAGAGCTTGGTCGGGGGAGGGGCGTCCGCCATTGCTCAGGCTTGAGTAGGTGGTTTTACATTCA
CAGTTTAAACAAAGGCACTGGGAAGTTCGAACTGGGCAGAGCCCACTGCAGCTCAGAAAGGCTGCTGGGGCCAGACCGCCAGATTTC
TTTTCTCTGGGCAGAGGATCTCTGACAAAAAGGTAGCAGCCCCAGTCAGGGACTTATAGATAAAACCTCCATCTCCCTGGGACAGAG
CACCCGGGGAAAGGGGTGGCTATGGGCGCAGCTTCAGCAGACTTAAATGTCCCTGCCTGACAGCTCTGAAGAGAGCAGCGGACCTCC
CAGCACAGTGTTTGAGCTCTGCTGAGGGTCAGCCTGCCTCCTCAAGTGGGTCCCTGACCCCTGTGTATCTGGGAGACACCTCCCAGT
AGGGGCCGACGCACACCTCATACAGGAGAGCTCTGGCTGGCATCTGGCAGGTGCTCCTCTGGGACAAAGCTTCCTGAGGAAAGATCA
GGCAGGAATCTTTGCTTCCGCTGGTGATACCCAGGCAAACAGGGCTGAGAGTGGATCTCTAGCAAACTCCAGCAGACCGGCAGCAGA
AGGGCCTGACTGCCAAAAGGAAAACTAACAAACAGAAAGGAATAGCACATCTGCTTAAAGACCCCATCTGAAGGTCACCAACATTAA
AGACCAAAGATGGATAAATCCACAAAGATGGGGAGAAACCAGCTGAAAAAGGCTGAAAATACCAAAAACAAGAACACCTCTTCTCCT
CCAAGGGATTACAACTCCTCACCAGCAAGGGAACAAAACTGGATGGAGAATGAGTTTGACGAATTGACAGAAGTAGGCTTCAGAAGG
TGGGTAATAACACACTTGAGCTAAACGAGCATGCTCTAACCCAATGCAAGGAAGCTAAGAACCTTGAAAAAAGGTTAGACGAATTGC
TAACTAGAATAACCAGTTTAAAGAAGAACATACATGACCTGATGGCACTGAAAAAACAGCACAAGAACTTCGTGAAGCATATACAAG
TATCAACAGCCGAATCAATCAAGCAAAAGAAAGGATAGCAATGATTGAAGATCAACTTAATGAAATAAAGAAACAAGACAAGATTAG
AGAAAAAAGAATAAAAAGGAACGAGCAAAGCCTCCAACAAATATGGGACTATGTGAAAACACCAAATCTGTGTTTGATTGGTGTACC
TGAAAGTGACAAGGAGAATGGAACCAAGTTGGAAAACACTCTCCAGGATATTATCCAGGAGAACTTCCCCAAATTAGCAAGACAGAC
CAACATTCAAATTCAGGAAATACAGAGAACACCACAAAGATACTCCTTGAGAAGAGCAATCCCAAGACACATAATCTTCAGATTCAC
CAAGGTTGAAATGAAGGAAAAAATGTTAAGGGCAGCCATAGAGAAAGGTCAGGTTACCCACAAAGGGAAGCCCATCAGACTAACAGT
GGATCTCTCTGCAGAAACCCTACAAGCCAGAAGAGAGTGGGGGCCAATACTCAACATTCTTAAAGAAAACAATTTTCAACCCAGAAT
TTCATATCCAGCCAAACTAAGCTTCATAAGCGAAGGAGAAATAAAATCCTTTACAGACAAGCAAATGCTGAGAGATTTTGTCACCAC
CAGGCGTGCCTTACAGGAGCTCCTGAAGGAAGCACTAAACATGGACAGGAACAGCCGGTACCAGCCACTGCAAAAACATACCAAATT
GTAAAGACCATTGATGTTATAAAGAAACTGCATCAACTACCAGGCAAAATAACCAGCTAGCATCATAATGACAGGATCAAATTCACA
CATAACAATATTAACCTTAAATGTAAACATGCTAAATGCCCCAATTAAAAGACACAGACTGGCAAATTGGATAAAGAGTCAAGACCC
ATCAGTGTGCTCTATTCAGGAGATCCAACTCACATGCAAAGACACACATAGGCTCAAAATAAAGGGATGGAGGAATATTTACCAAGC
AAATGGAAAGCCAAAACCAAACAAACAAACAAAAAACAGGAGTTGCAATCCTAACCTCTGATAAAAAAGACTTTAAACCAACAAAGA
TCAAGAGAAAAGAAGGGCATTACATAGTGATAAAGGGATCAATGCAGCAAGAAGAGCTAACTATCCTAAATATATATGCATCCAAAA
TACAGGAGCACCCAGATTCATAAAGCAAGTTCTTAGAGACCTACAAAGAGGATTAGACTCCCACACAATAATAGTTGGAGACTTTAA
CACCCCACTGTCAATATTAGACAGATCAATGAGACAGAAAATTAACAAGGATATTCAGGACTTGAACTCAGCTCTGGACCAAGCGGA
CCTAATAGACATCTACAGAACTCTCCACCCTAAATCAACAGAATATACATTCTTCTCAGCAACTCATCACACTTATTCTAAAATTGA
CCACATAATTGGAAGTAAAACCCTCCTCAGCAAATGCTAAAGAATGAAAATCACAACAAACAGTCTGTCAGACCACAGTGCAGTCAA
ATTAGAACTCAGGCTTAAGAAACTCACTGAAAACCACACAACTACATGGAAACTGAACAACCTGGTCCTGAATGACTACAGGGTACA
TAACGAAATTAAAGCAGAAATAAAGATGTTCTTTGAAACCAATGAGAACAAAGACACAACATACCAGACTCTCTGGGACACATTTAA
AGCAGTGTGCAGAGGGAAATTTATAGCACTAAATGCCCACAAGAGAAAGCAGGAAAGATCTAAAATTGACATCCTAACAACAAAATT
AAAAGAACTAGAGAAGCACAGCAAACAAATTCAAAAGCTAGCAGAAGACAAGAAATAACTAAGATCAGAGCAGAACTGAAGGAGATA
GAGACACGAAAAACTCTTCAAAAAAATCAATGAATCCAGGAGCTGGTTTTTTTTTTTTTTTTTTTTTTTAAGATCAACAAAATGGAT
AGACCACTAGCCAGACTAATAAAGAAGAAAAGAGAGAAGAATCAAATAGATGCAATAAAAAATGATATAGGGGGCCGGGTGCTGTGG
CTCACACCTGTAATCCCAGCACTTTGGGAGGCCGAGGCGGGCGGATCACGAGGTCAGGCGATCGAGACCATCCTGGCTAACATGGTG
AAACCCTGTCTCTACTAAAAATACAAAAAATTAGCCAGGCTTGGTGGCGGGTGCCTGTAGTCCCAGCTACTTGGGAGGCTGACGCAG
GAGAATAGTGTGAACCCGGGAGGCAGAGCTTGCAGTGAGCCAAGATCGCGCCACTGCACCCCAGCCTGGGCGACAGAGCAAAACTCT
GTCTCAAGAAAAAAAAAAAAAAAAAAAAAAGATATAGGGGATATCACCACCAATCCCGCAGAAGTACAGACTACCATCAGAGAATAC
TATAAACACCTCTATGCAAATAAATGAGAAAACCTGTAATAATGGATAAATCCTGGACACATAACACCCCCCACAAGTCTAAACCAG
GAAGAAGTTGAATCCCTGAATAGACCAATAACAAGTTCTGAAATTGAGGCAGTAATTAATAGCCTACCAACAAAATGTCCAGGACCA
GATGGATTCACAGCCGAATTCTACCAGAGGTACAAACAGGAGATGATACCATTCCTTCTGAAACTATTCCAAACTATAGAAAAAGAG
GGAATCCTCCCTAACTCATTTTATGAGGCTAGCATCATCCTGTTACCAAAACCTGGCAGAGACACCACAAAAAAGGAAAATTTCAGG
CCAATATCTCTGATGAACATTGATGCAGAAATCCTCAATAAAATACTGGCAAACAGAATCCAGCAGCACATGAAAAAGCTTATCCAC
CACGATGAAGTCAGCTTCATCCCTGGGATGCAAGGCTGGTTCAACATACACAAATCAATAAACATAATCCATCACATAAACAGAACC
AGTGACAAAAACCGCATGATTATCTCAATAGATGCAGAAAAGGCCTTTGACAAAATTCAACACTCCTTCATGCTAAAAACTCAATAA
ACTAGGTATCAATGGAACATATCTCAAAATAATAAGAGCTATTTATGACAAACCCACAGCCAATATCATACTGAATGAGGAAACACT
GGAAGCATTCCCTTTGAAAACTGGCACAAGACAGGGATGCCCTCTCTCACCACTCCTATTCAACATAGTATTGGAAGTTCTGGCCAG
GGCACTCAGGCAAGAGAAAGAAATAAAAATAAAGGTTATTCAATTAGGAAGAGAGGAAGTCAAATTGTCCCTGTTTTCAGATGACAG
ATATTTAGAAAACCCCATCGTCTCAGCCCAAAATCTCCTTAAGTTGATAAGCAACTTCAGCAAAGTCTCAGAATACAAAATCAATGT
ACAAAAATCACAAGCATTGCTATACACCAATAACAGACAAACAGAGAGCCAAATCATGAGTGAACTCCCATTCACAATTGCTTCAAA
GAGAATAAAATACCTAGGAATCCAACTTACAAGGGATGTGAAGGACCTCTTCAAGGAGAACTACAAACCACTGCTCAAGGAAATAAG
AGAGGACACAAACAAATGCAAAGACATTCCATGCTGATGGATAGGATGAATCAATATTGTCAAAATGGCCATACTGCCCAAGGTAAT
TATAGATTCAATGCTATCCCCATCAAGCTACCAATGACTTTCTTCACAGAATTGGAAAAAACTACTTTAAACTTCATATGGAACCAT
AAAAATCCTAGAAGAAAACCTGGGCAGTACCATTCAGGACATAGGCATGGGCAAAGACTTCCTGACTAAAACACTAAAAGCAATGGC
AACAAAAGCCAAAATAGACAAATGGGATCTAATTAAACTAAAGAGCTTCTGCACAGCGAAAGAAACTATCATCAGAGTGAACAGGCA
ACCTACAAGGTGGGAGAAAATTTTTGCAATCTGTCCATCTGACAAAGGGCTAACATCCAGAATCTACAAAGAACATAAACAAATTTA
CAAGAAAAAAACAACCCCATCAAAACGTGGGCAAAGGATATGAACAGACACTTCTCAAAAGAAGACATTTATGCAGCCAACAAACAT
ATGAAAAAAAGCTCATCATTATTGGTCATTAGACCACAAATCAAAACCACAATGAGATACCATCTCACACCAGTTAGAATGGCGATC
ATTAAAAAGTCAGGAAACAACAGATGCTGGAGAGGATGTGGAGAAACAGGAATGCTTTTACACTGTTGGTGGGAGTGTAAATTAGTT
CAACTACTGTGGAAGACAGTGTGGCGATTCCTCAAGGATCTAGAACTAGAAATACCATTTGACCCAACAGTGCCATTACTGGGTATA
TACCCAAAGGATTATAAATCATTCTATAAAGACACATGTATGTATGTTTATTGCAGCACTATTCACAATAGCAAAGACTTGGAACCA
ACCCAAATGTCCATCAGTGATAGACTGGATGAAGAAAATGTGGCACATATACACTATGGAATGCTATGCAGCCATAAAAAAGGATGA
GTTCATGTCCTTTGCAGGGACATGGATGAAGCTGGAAATCCTCTTTCTCAGAAAACTATCACAAGAACAGAAAACCAAACACTGCAT
GTTCTCACTCATAAGTGGGAGTTGAACAAGGAGAACACACGGACACAGGGAAGGGAACATCACATACTGGGGCCTGTCGTGGGGTAG
GGGGTAGGGGAGGGATAGCATTAGGAGAAATACCTAATGTAGGTGACAGGTTGATGGGTGCAGCAAACCACCATGGCATGTGTATAC
CTATGTAATGAAACTGCAAGTTCTGCACATGTACTCCGGAACTTAAAGTATAATTTAAAAAAGAATAAATAAAAATAGAGAAGTGTG
CCTGTAATCCCAGCTACTCAGAAGGCTGAGGCAGAAAGATTATATAATACCAGGAGTTTGAGGCAGCAGTGAGGTATGGTCATGCCA
CTATACTCCAGCCTGGGCGACATAGTGAGACCGTCTTTTAAAAAAATTTTAACATTTAGCCGGGTATGGTGGCACATGCCCCTAGTC
CCAGCTACTCAGGAGGATGAAGCAGGAGGATGGCTTGAGCTCAGGAGTTTGTGGCAGCAGTGAGCTATGGTCTTGCCACTGCATTCC
AGTCTGGACAATGGAGTAAGATCCTGTTTCTAAATATGTGTGTGTGTGTGTGTGTGTGTATAATATGTGTGTATATGTTGTGTAATC
TTAAGTGAATCTAATAACTGCTCTAGTTTACTCTGCAAATGAAAAAACTAGACTAGACAACCTCTAAAATCTTTCTTGATTCTAAAT
ATCTGTGTCTCAGTTTAGCTCCCATTCCCTTATTCCCAAGTCCAATGCACATCCCATCACTTTGTTTCAACTTTCATGGCCGAACTG
CAGGTGGGGTGGAGTGTGCCTCTAGGAAAGTGGAAACGGCTGTGGCTTTTCCACTGTGGCTTCAGGGAGCCCTGGCCTTAATCAAGC
TGCTTTCTAGAGCTGTGTTGCCTCTGAGCCTGTTTCCTCATTTGTATGATGGGGATATCATTGTCTCCTTCACAGTGTGATGACAGT
TAGTCATGTACAGAAGGCACCTATAGCTCCTGGCACAGGCTGATGAGCAACAAAAGTTCACCATTATCACTTTCTCAGTGTCTAGGG
GACATTTCATCCCTGAGCATCTCTGGCCATTTCAGGATGTGCTCACTGTCTCTCATTTGCTCTGGTCCCAGTGACCTTGTTAGCTCC
TGGCAATGTGTGTGCAGGCCCTTCTGCCTACCCAGTCAGGTGATGGCCTGGTGAACAACAGCAGAGTAGGCCCATGCCTGATGCTCA
GTCATTTCACCATCTTTTTCCTGCCAGGTGCCATGTCCAAGATACAACACCTACATCAAGGAACTCAGAGTCTGAAGTCAAGGCTGC
AGAAACTGTGGTCTAGCTTTCTGACCAAAGCTGGGTTTTGTGCTTCTATGGCAGTGTAGATTCTTTTACTTGAACATACTCAAAATC
TTAGTTTTTAATTGGTGATTCTTTTTAGGATTACAGCAGTTTTTGGTCTCAAAGCCAAAAGATTCTGAATTATTACAGATCTTGTGT
TATTTTGTGGCTGTATAACATCTCTTAACCAACCAAACAAGATGAGTAGGTTAGAGATTAAACTGGCTGCAGAAGTCTAAATGAAAC
TGAATCCCCAAGTTCTGAGACGCAGTGAGGAGAAACTGTATTGCCAATAGATCTTCTCATCCTGCATTTGGAGTGGTCGGGATTCTG
CAGTCAGATGCCTGGGTTTGAATTGGGGTTTTCCCAGTCACTAGGTAGGTAGCAACTCTAGTCCAGGCTACTTAATGTCTCGTAAGC
TCAGTTTCCACAGTTGGAAGTGACGATATGGTGTTTACTTCAGTGGTTGTCCTGAAACTTTATTGAAATGACACAAGTTCGTAAACT
GTGCCAGTTCCATTGTAAGGATGCAATAAATGTTAGCTCTTGTTTTTATTAATGACGAGGATATAATGTTTAAACTCAATGCCCACT
TCGGGCAGTTTTTTACCCTGTAGAAGCAGTTGTAATCATTTTCAAATCAGGCTTGCTACACCCTTCCAGCTGGAAAGAAAAGTCTCT
AATTTCATTGATTTTCGAGGGCACATGCTTTAGTTGAATGAGTAGTAACCAAATGGAGATATGTAGTAGGCGGTTAGAAGTGTTAGC
ATCTTTGGGATCATGTGGTCAACCACATTATAAATGAATGCTTGGATGTAAGGGCAGACCTGCATCTGTGTGTGTGTTTTCAGTGGG
TGGATGGGCCATGTGGTATCACAGCGTGCCACACAGGGATTTACGGTGTCAAGAAGCATTTGTGGTACAGAGCTTGGAGTTTGGAAA
GTAAGGCTTCCTTGTTTTGCATCCTGGCTCTACCACTGTCAATCTGTGTGACCTTGGGTGAGTCACTTAACTGTTTCCTCACCTGTT
AAATGTGAATAATAACAGTACCTCTCCCCTTTTGTGAAGACTGAATGAGATAAATGAAAAGCTGTTTGCAGTATGGTCATACTAAAA
TGGGAGCAGTCAGTACCCTCCCCCCATTTCCAGCAGAGTGGTGGGCAGGTCTAGCTAGCCCTTGTACATACACCTTGCAGAATAGTT
TATAGTAGTGAGCATAGCTGGTAAATGTACAATAAAAAAAGTTCCTGTTTTTCATTTCTACCTGATTGAATGAGACACCTATTTAAT
GCAAATTTCAATAATGCTCTGATACTAACTCTGTTCAAGTTAGTAAGAGAGCTTTGTAGAGTTATTGTGACACCTGGGTTAAGTATG
GCTGACTTTGGGTTAAGTATGAGGTCTAATCATCCCAGTCTGTCCTCTAGATTTTTTGCTGTTTTGTTCAATATAATTATGTAAACA
GGTATTCTCTATGTAAACATTTGTGAAAGTTAAAATACCAAAGCAGATTAAATATAGAACTAGACTGGATGCCTACATCAGTGTTTT
AGTGACAGCCCCCCCCCCCCCCCCCCTCATCTGTTACATTTAATTATTTTGGCTAGTTTCAGTTCTGTTGGTTTGATAGTATTCTTT
TTAGTTGATATATAAATTTGCTTGGGGTTTTATACCAGTCTTCTATTTCTGCATATTTAAGGAATATTTTAATAAATAATAACTTAT
GTCAACCTTGAGGATCTATGACTTTTTTCTTTTAAAGAGAGTTCATATATTAGTCCATAACCATTGAATAGATCGTTCAGAGTGTAT
GAAGAAGAGCATAATCCTGTGCACAGCTGGCTTTTCTGATTTGCAGGGTATTTAGTGTACATTTCTCTATCCAGCTCATATGCCGGA
CAGAGGAACACTTTACCCTTCTGGTCTCTCTGCCTGTTGGGCAATTGGGTTATTCTGGGTATGTGGAGGACCTTTTTTGATTCTTGT
TGTGAGGGATGCTTTCAACAGAAAAGAGAGGAAGAATATTTGTCTTTCATTGCTCAATACCCAGATATTTCATATTCATCAGAAATA
GCTACTTCTGAATTCTTTTTTCCTCTTTAAATTAGACACAACATTATCCCGTCTACTGTTATTTCTTTTTTACATTTTTATATTAGC
AGTCGACTGTTGGCATGTTCTCTCAGCTGCCTCTAGTGATGACATCCGGGCTTTTAACAGCCATGCTTTTCCAGGGAGCCCTGTGCA
CTTCATAGATTACACCGTTAGTCCTCACAGTTGTTCCCGGAGGTAGGTAGGATCAGACAGAGCACCTAGGGGTGAGAACAGAGGGCT
GGGGCGAGTCAGTCTGAGAAAAGCAGTGGGTTACTTCTTTTATGTTAGCATTTCTGCTTTAGGCAGAGTTCTGCATTTCCACCTATT
TTCATATTTTCTTCAAAGCATTTTTCAAATGATAAGTCATTAGCTTCATGGCTGCAGAATGAAGTAAGGGGATATTAATATAGCTAT
TGAGAAATAAGAACCTTCACTGAAATATTTTTAGGGTCGTTAAGAGCAGTTTTAACTCTTGGTTTGGGTGTGATTTTTGCCTAGAAT
TTGGAGTGTATTTAAATTGATTTTATATCTAAATTGAGTGATCAAAAGAAACCTCTCTAATGATATAGTTGTCTAATGTCTATTCAG
TGTAACTCTTTGAAGGTGCAAGATTAATATTCCTAAAGCCCAGCTCTGATCATTTCATAATTTTCTTTTGTGTCCTTTTTAACATTA
TGAGCTTTGGATTCAAAAGACCTGGCTTCATTTTATGGGCTGGTTATATAACCTGTCTGATTCTCACTTGCCTCCTCTATAATGGAG
GTAATAATACCCAACTCAAAGAGTGGTGGTTACAATTAAGTGAGATCATGTGTGTGAGATACTAAGCACAGTCGATGTCAATAAAGC
TTTAAAAAGGCAAAAAACAGTACAAAATCCAGATTCTTTGATGCTATGTTTTAGATGCTGCCTTGGAATCACCTCATAACTGAGTTC
TTATTTTCCAGCCAAATACAAAACCTCACTCTTCATTTTTCATGAAGCAGAACAACCAATACAATTTACATGAGGCTAAAAAGCTTC
ATGAAATAGTACTTTATCTTGCCACATGCTATAGCATTCTATTCCATTTCATTGAGGAAGAAAATGGCCATTAAACTGTGATATCAA
GCAAGTTGCAACCTGTTTAAAAAACAGTGGTCTAGAAGGCCTTCCATCTCCATTTCTTAGAATTCTATCCTCCACCCCCCTTGAGAC
CTGTACCCCCTCATGAAATCTTCCCTCATCTTGGCATCTGGTGGCCTTCTCTGAACTTATTTTTTCTCAGTCTCATTTATGGCATTT
ACTGCAAATTGGCATTTAGGTTCATTATTGATTCATAACTTATCTCTATGGTGAGATGATAAGTACCATCAGGGCATGAACTCCATT
GTGCCATCATTTATCAACCTGTGAACATAGCAAGATGTCCTGCCCACTGAAGCCCTCAGAGGTATTTGTTAGGAAATTGCCTGGTGT
CTTGGATGCTTCCTTACCTTTGTGCTGACATTTGAAAGCCGTAGAATTAGAGTTTGTGTGTTTACCCTTGGGAGCGGGTTGCCTCTC
GCCATGACTACTATCTAGTTCAGATGTTTTCACCTGGCCTGCCTTGGATTTCTTGTGTATGTGAAAAAGAATGACATTAGGTTGAGA
TTGGTATTCTGGCTTCTAAATGGAGATTCTCAATTTTCCTGTTTCCTGACTTGACTTTGTTGGGCCTCAGAGGTTCCTTTCAGTTCT
AACGGTGTTTGAGCAAAAATCAGCCTCCTTTCTGATGAGACCCAGCCTGATATCTTTAAACCTTGGACCTTTGCAGCATGTAACAGT
TAATGATTTTAGCCTATGAGACCTGCACTTTATAGCCTAGGACACCTTTGATGTTTCTGTGTATGTACACGGTGATCTGTAGATTCT
ATATGTGAGTATATTTATATGCAGTATTTTATTCATATAGAGCCTTTCATCTACAATGAGAAAATAGTATACAGACAAGAATGGAAT
ACTTATATCTTAATTATACAGATACAGTAAGTCCTCACTTAAGATCATCTATTTGGAAACTGTGACTTTAAGCAAAATGACATGTAT
GTATAAGAAATCTAATTTTACCATAGGCTACTTGATACAGACAAAAGTTGAAGTTGCTAGGACATATTTCTGGTCACAAAAACATCA
GCAAACTAAGTAAAGATTCAAAACACTTTTAATATTAAACATTGAAATAAATGTGAGCTATATATACAGAACTCAGGAGGTGGAGGT
TGCAGTGAGCCGAGATCTCACCACTGCACTCCAGCCTGGGGGACAGAGCAAGACTCTGTCTCGAAAACAAGAGAAAGGATTAATAAA
AACAAAATAATTATTTACTCAATGATTCCATGATTTTAGTTCAGGGTTGAGAGTGGCTGGAGTCTGTCCTGGCATATCAGGGCACAA
GGCAGGGACCCACCCTAGACAGGACACCATTCCATCACACATCCACACTCACTCAGACAGGGACCGTGTGGACACTTCAGTTCGGTT
CACCTAATGCACACATCTTTGGGATGTGGGAGGAAACCAGAGTACCCAAAGAAAACCCATGCAGACGTGGGGCAAATGTGCAAACTC
TGCACAATGGCCCCGGCCTCTCATCAATGTTACTGAAGGAAACGACATTGAACAAAATGACATTTCTCGAAGACCTGCTGTACACCA
CATCTCACATTTGGAGCCCAGTTTTGAGAACCTAGTGATAAGATTTTAGGAAGAGCAGGACCCATTTTATGTTTACACAAATTACCT
ACTCAAGGGTCAACCTCATTTAAAGGAACTGATTTAATTTAGGAAAGCTGTTTCTTTCCCTTTTAAATCTAATTTAAGAAGCTTGAC
TGAAATAGTAATAGATACCAGAGAAGTGACTGTGCAGCTACGTAAACTAACAGACCTGGCCATAGAAACATCTCTCTTGGGACAATA
CCTTGATAAAATACATATCAGAAATATCTAGGAAATTATAGATAAAAAACAGATCATTAACAATTTAATGACACACAGTAAAAAGAT
TTTTAAAAACATAACTGCATAGTAAAGTCTGGGAAAAGTTACTGACTGGTACACAGGTGTAGGCAGTAAATCTATTCATAGTCAGAT
TTTTCTCTTTTGAGCATTGAACAGTTAATAGGGACTTATGAAATGAGTTTTGTTAATTCAGTACTCAGAATTCTCAGACAAGCTTCC
TTATTTAGATTCTTTCCCATCTAGTCTGCCATTTACCAGAGCTAAAGTGTGTGTCAAAGGAAGCAGGATTTATCTTAAAGGAGATTT
GGAGGAAATCTTCCTTCAATATTTAAGTGTTTGACAAAGTCATGCTCACATGGACGTCAGTTTTAAGAGGAGGAAGTTTAAAATCAG
GTGGCTTATTTACGTTACTGGCTGAAAGCCTGTCTGATAGGATTGACATGGAGCACTAATTAATCACCTAGGGTCTCCTCATTTACT
AATCATATTGCACAAAACTTCCCTGCTGACTGAGGCTCAAGGGCAAACTGTGGGCTTCCAGCCAGCTTATTTTTCATAAGCATTATG
CTACCACTGTAAATTAATGTGAACCTAATATATTCTGTCAGCGGTTTACTTTCCCATGGCGACCCTCTCTGGTTAAACAGCTTTAAA
TGAAATCTTGTATCAAAAATGAAAAGAAGTTTCTTGCAAAAGGAAAAAAGGATTTTAGGTCCTCTTTGCAATCTTTTTTCCCCATAG
TATATGCTATTTCGGTTCTGATACAAATCTGCTAGATTAACTGAATAATGCACAGATGATGTGATAGTTTCTTTTTGTTATCTGAAA
AGTGACCTTATATTGTGCTTAGGTAAGAGACTTTTTAATTACACTTTTAATGGCAATAATAGTGTGTTTATTGAGTGCCTACTATAC
ATAAACACATACCTTAACTCATTGATCCTCATATCAATGCTATGTGAATGCTAGGCAATAGTCTGTATTTATGGGAGAGAAAAATTG
AGAGTGTCAGCTTGCCCAGGCCACATCCATTAAATGCTGGGGCCCAGATTTGAACCAAGGTTTGTCATCAGAGGTCAGGCTCATTTG
GCTGTGTCACTATTTATAATTTGTATGGTCCCTTTATACTTTCTGTTTTAACTTCAGGAAATTAGCAGTCCTCTTAATTTTTAAAGA
TAAAAGCTGGGCACGGTGGCTTATGCCTGTAATCCCAGCACTTTGGGAGGCCGAGGCAGGTCGATCACTTGAGGCCAAGAGTTCATG
TTGACCAGCCTGGCCAACATGGCGAAACCTTGTCTCTACTAAAAAATACAAAAATTAGCCAGGTGTGGTGGTGCACACCTGTAATCC
CAGCTATTCAAGAGGCTGAGGCAAAAGAATCACTTGAACCCGGGAGGCAGAGGTTGCAGTGAGCCGAGCACGCGCCTCTGCACTCCA
GCCTGGGCAACAGACGGAGACTCTGTCTCAAAAAAAAACCATAATTTTTATTTCCATGTTGATTTTTTAATATTCATCTGAATTAGT
AAATCATTTTAGAAGCTTGCTGATCTCTAGCACATGCTTTTATATCTTCAACTCACTTTAATGTCATGCCACTTTTATTCAGAGAAC
AAAAAGTTATGGATGTCTTTAAACCAGAGGAAATAAGGGACCCTTATTCACATCCCTCATGCTGTTGTTCATCAGGAGACTCTTCCA
AACAATTCTAACAGAAAAGGAAAATTCCAGAGTGCACACACATGGATAGTTTCCAAGGCAGTGCTTGTTGAGAACCCAGCCTTCTCT
CCTGAAGCTGTGAATAGTTTGTCTTATATGTAAATTAATTTCCAAGAGTCTGTGCATTAAAGTCTTATATAAAAGAACATATAAAAG
AATGATTTGTCAAAACCCATTTTCCTTGTAAAAAATACCTAACTAAGGAGAGAGGATGATATTCGGTGGCTTCTGTGGGCTCTGTGT
TAAGCCAGTACTTTACTTCTTAGTATGCAGTGCTGATGTTCTTGGTTGAAAGGAATCCATTCCATTTCTTTTTATAGTATCATGAAA
AGTTGGAAAGAGGAAAAACTGAGCTGAACATACAGAATAAATAACTAAATGGTGTCGTCAGGCTGCCCTTTTGTTTTCAGACATCCC
CCCTCCTTCCTCCTTTGTGAGTCACTGAGCCAGTAAGATGGATCAAAGATGCCTCAGTGTATAGAACATTTTATGTGGAGGTTATTT
GTCATCCACAACAAAGGACTAATTGCCTCTGTGCCTTCTTTCACATGATAATCAATGGATTGGGATCTCCTGATTATGCCCCATGCA
TCCTGCATTCATCTCTCAAGATGTTTTAGGTTTTGATTGAATTTATAAAGCATCAGAAAGATGACGTCAGTTCAGTGCAACTATTAA
GGGGATTTTAAACATGGGTATGGAAATATGAAAACAGAGCTTGAAGTGAAGGCACCTGTCATCATTCTTTTTGTAAATGGTTGATTT
GATCGGAGGTATAACTTCTATGATAGCTATTAAGAAGGGTCCTGGCATTTTTAAGATGGAGCGTTTATTTCAAAAGGCCCACCAGGA
GAAAATTCACTTCCTGTTTCAGCGTGGACTTGAGGTTCTCAGAGATGACTTCTTGGATGACTTTGTTGCTAATGTTATCAATCAGGT
TTCATTTTAAAGAAGCTTTCTGAATTCTAACAAGAAGTCAGTGTACTCTGAGTATAGTTTTCTGTAGTGGATTTGCCTAATGTGGGG
GTTGGGCACATAGGCCACGGGAGCGTGTAAGTGTAGGAAACACCATTCAGCTCATTCTGTACAATGTCTGAAGCTTTGAAGTTACAT
TCTCCTGGCATTTCAATTATTTCAAAGGCAAGATGTTTACTAAAATGTACCTGAAACATGGAGTTATTTCTTAGGAACTTTTAATAT
TTACAGTTAAATTAGAACACTTTAAAATGAAAGAATGATATATTGACTGTAGCACAGACATCTGTTTAATAACAGTCACTTTTCCTC
TCTTTAAATGTATAAATGTCCTGTGTTTCTTAGAAAAGAAAGATTTCATCAGTGAAACAAACAAAAAAAACAGAACCCACAAAAACA
AAATAAAATTCAAAAACACACACAGATATATAGGCATAGAGAGAGCATTTGTGATAAGTGTTTTAAAAATAGCTATTAATATCTCAG
CAGCCTCCTCAGCTCTGATGGTGCTGACATCCCACAGGGCAAGCTGAAGCAAGCCAGGGGGCTGGAGAACAATCTAAGACACCAAAC
TCTTCTGTGGAACAATAACACTGAGTGTCAAGGACTGTGAAGGGTCTGAGATTTCACCCTGCATGCCAGCAAACAAGTTATCCTGCC
ACAGTGTCACAAATCCTGGCAGAAGGCATCACACTGCTGGGGCAGACACATAGGACTTTATTAATCACAGCAATGCCAGGAACCAGA
GGATCAATATTTTTGTACACTTTCCCTGAGCCCTAATTCTTACAGGGTGGTGCAGAGAGGGCCAGGTGATACATAGTGAGTTGTATT
CTAGGAGAGGAACCCTGAACTTAGGGAACTCCGATCTTTTATAATGGGCAGTAAGTCTCCCTGACCCTTGCTCTGTAAAGAAACACT
GAATCTTTCAAGCCTATCTGCTGTTCAAATTTTCTTGAAAAGATAGTAACGAAGGACTCACTTGTAATGTCAGTGCCTTGCTGATAA
GGTGGCTCACACCTGTAATCTAGTGCTTTGGGAAGCCGAGGCAGGTGGATCACGAAGTCAGGAGTTCAAGACCCGCCTGACCAACAA
TGGTGAAACCCTGTCTCTACTAAAAATACAAAAATTAGCCAGGCGTGGTGGCACGCACCTGTAATCCCAGCTACTCAGGAGGCTGAG
GCAGGAGAATCGCTTGAACCGGGGAGGTGGAGGTTGCAGTGAGCCGAGATTGCGCCACTGCACTCCAGCCTGGGCGACACAGCGAGA
CTCTATCCCCCCCACCCTGCCCCCCAAAAAAGTGTAGATGTGAGAATTGTCTCCCCACACAAACTCTTCTGGAAATAGGCACCAGAT
AAGTACCGATTCCATGAATGGCTAATCTGTGCTGCCTTCTAAAGGCAGTGCAGGTACCAGATGACACAGCCATAACTGCAGCGTTAT
ATCTCTGTTATTTTAAGAAATGGATAAAAGGTGGTATTTTAATTTATCCTGTATCTATGCAACATATTCTTTTCCAAATGTCTTCCC
TGTGTTTGACAGTCACAGTCTTGTGACATTGCAGCAGGTGTTATTACCCATTTACACTTGAGGAGACTATCATTCAGTGACTTGACC
AAGGTCACACACAGTTACTCTGCAGCTCGCCACTACACCTGGGGGCTCCTGACTCCCCGTCTGATGGTCTTTGGAACACATTGTGTA
ACACTGCCTCCCTTTTGTCTCCTACGTAAGGTAACGGATATTTCCAGATATTACAGAGGTGCCCCCAGGCCAGTCCTGAGTTCCTTT
TTGGCAGTTTCCGTGCCTCCCAACTCATCTTACCTAGCCACACAATGCCCAGTGTCTGGTGGTCATGCTGGACTCATTTGAGTTGAG
GGAAGTTACTTTTTTAATGCTTTCTTGTTGCTCTATGTTTTAATTTTCACTTAACGGAACACAAACAGTCTCCACTGTTGCCACCCC
TTTGCTTAGTAGAGCTGGCCCCTTCCCCTACTTAATGTGTAATTGCTCTATGGGCTTTGGTGGCACCTCTTTCTTGGTTTCCTGCTG
CCCTTTTATCTAATTCTTCTGAATCTCCCCTTTTGGTCCCCTCTGTACACTGCCCCATACATCTTCATGCTCTCTAAGACAATGCCT
TGATCCTCTTTCCTCCAGACTATATGTAGCCTTAATGATAACCACAGTCAGTTATTTATTGAGCCAGGCATATGCCAGGCACCTTTT
CATGTGCCACACACTTTAAGCACGTTATTTGAGCACATGCGTTATCTCATTTAGTCTTCCCAATGACCCCACGAGATAGATTCCACT
TGTCTCTGTGTTTTGTAGAGGAAGAAGTAGAGGACTGACAAGTTAAATTACTTGTCTGAGGTCACACAGCCAGTAATGGTGGAACCT
GAATGCTAAGCTAAGTTTGACTCTAGAGCCAACTGCTGTCCAGCCCCCAGATTTCCTTGTGCAAGAGTCAGAATCTTTCCACATCAG
TGTCTGACATCTGTCTTGGTAATAACGCCTAAAACTGAAATCTAAAACTGATATTTCTCTCCAAATATTCTCCACTTCTAGTATTCC
TAACTTTGTAAATGACACCAAAAGCCTGCCAAGTTGTTTAAGCTAAAAACTGGGACGTTTTCTTAGACTGCACTCTTTTCTTCACCT
CCCAGGACATTCCCCTTCATCCATTCATTTTTTTCCTTGCCTTTCCTCTTGTTTTTCCTTTCCTTTCCTTTCTAACAGACTCGCTGT
CTCACTCAGGCTAGAATGCAGTGACGTGATCATACCTCAATGTAACCTTGAACTCCTGGGCTCAAGCGATCCTCCCACTCAGCCTCC
CAAGTAGCTGTGACCACAGACACATACCACCATGCCCGGCTAATCTTTTTAATTTTATTTTCATATTTATTTTTTATATTCACTGTG
TTGCCCAGGCTGGTCTCAAACTCCTGGACTCAAGCAATCCTCCCACATTGGCCTCCCAAAGTGCTGGGACTACAGACTGAGCCATTG
CACCTGGACCTAAAGGCCCATAATTTTAACCTTGTACCCTGCTGCTTCCTGCCACTGAGTCCATAGAGTCTCTGCCTTTTCATCTGC
TGTCTCCATCTTGCTGCGGCTGCCTCTTCCTTTTTCAGCTGATGTCATTCTCACCAAATTAGTAGCCTCCCAATTTGTCTTGCTACC
TGCAGTCTCATCTCTCTTCAATTCATTCTTTGTACTGTGCTGGAAAGATGTTTCTGAAATGCAAATCTGATAACTTGTTTTGCTTAA
AATCCTTGGTTGGCTTCCCATGGCTTGCTGGTCCCTTGGTACCCATAACACATACAGCTATGCTGATCAGGCCTTAACCCTTCTAGC
CCAGAGGTTCTCAAACTTCAACACATGTCACAATTACCTGCAGGGCTTGTTAAGAAGACAGAACTTGCTGGGCCCTGCCCCCAGAGT
TTCTGATGTAGTAGGTCTCGAGCTGCCTCAGAATTTGCATTTCTGACAAGTTCCCTGGTGCTACCACTGGTGCGGGACCATAGCCTG
CCTAGTGCCTTGTCTGGCTCTCTCTGCTCCAGCCCTGGAACCACTTGCAGGTTCCTGAAGGGAGATGCTGTTTCAGGCTTTGATCCT
TTTGGATAAGCTGTTGCCTCTACAGGAGATAGTCTTTCTATCGCCGTCTCTTTAAAATACAGTTCAAGTGTCACTTCCTAGGAAGCT
TCCTCTGACCCCTGCAGTGATCTCATAGTTTCTGTGGCAACCCATGTGCATCTCTGGGAATGCTCCATTTCTGTAATTGAGTTGTCA
TTTTATTTATGTATCTCCCACCCACTCCCACCAGACCATTAGCTCTTTAAGAAAAGATATCATTCATTTTCTTTTTCACCCTTCTAT
CCCTGGCACCTAGCATGGTGGCTGGTATATACTGAAGATATTCATTAAATAAGTTGAATGATATAATCCTACTTTTACCTTAAAGAA
TAATTCTATTTTGGTTTTTATTTGCATTAACCTTTATCATACCTTGTTTTTAAATGTCAGGATAGACTGGGTGTGGTGTGGCTCACG
CCTGTAATCCCAGCACTTTTGGAGGCTAAAGGGGGGAGGATCACTTGAGCCTAGGAAAAAAAAAAAAAGTTAAAAAAAAAGTCAAGA
TGTTTCAACATAAAAACAGTGTGCATCTGACAGTATTAAACTTTCGAAGCTACCATTAGAGAGAGCTTATAACTCAGCTGTCTAATG
CAAACCGCAGTTTATTCCTACAAAGGCTGATGTTTGTCTATAGATAACTTACTTCTCATGCCTCTGATTTGAAGTGCTTGAGTTATT
CCTCAGTCTTTCTCAGCCTTCAGAACATTTCTCTGGTATGTTTTTCAGCCTTGGATTTATCTAATCAGGGAGTTACAGATGGAATAG
TGCAAGCCTTTCTTTTACTGAGACAAGCAAAGATTAGTGGGCTGTAAACTTGCAGCCTCCATGTTTCCATCCAAAAGAGGATGGCTC
ATCCCTCACTTTCCCTTTTACGTGCCTGCATTGCTTGGGTAATTTCCAAGAAGAGTATCAGTTTGATTGCTTGCACAGCCCTCCCTT
CTCTCCCCTCCCCATCTCAGATGACTAGTCCTTGCCAAAATGTCAAGTTCCAAACCCTAACAAAAGAGTTTGCAAACAAGACTGTCC
CTGGATTGTAGACATTTCTTCAAGGGGTGCCTGGCCTTCTCCTGGTGTAGTGCAAGTTCAAAGGCAAAGAGCCCTTTCATCCTCTGG
GAAAACAGAGAACACAGTTCCAAAAATGCTTGGATACCTCCAAATAGACCTCCTCCCCTGCCTCACCCCTTTCCCAGGCTCTGCTAT
GTGGTGTTTTTCAGGAACCATTCTGTGGATTTTAGCCATATCATTCTGCTTCCATAGTTCAACAAGCAAGCAGCAGACAGCTCTTTC
CTGCAGTGGATGTGTGGCTGAATTATATATTCCTTACACAGCAAGTAACAACATCTTGCCCAGTAGTTCAGAACAACTGGTAATCTC
CATTCAATTATTTGGAAAGAAATTTCCCTCTGCCAGAGCATGATTAGTCTCTGAGCACCATCAGTCTCATGCCTGTTTTGCATATCT
TGTAGCTTACTGTAAGCTTGTCACAGGATTAAAGCATGAAAGCAATTGATCTGGTCTCTTATTTATGTCTTAGGACCAGTCTTAATC
TGTTTTGTGGTGTTGCTACAATAGAATACCCGAGACTGGGTGATTTATAAAGAAGAGACATTTACTTAGCTCATGGTTCTGCAGGCT
GGGAAGTTCAAGGGCATGGCCCTGGTTTCTGGCAAGGGCTTCCATGCTGCATCATAACATGGTGAGATGCACAAAGGGGAGACAGAC
ACGTGCAAAGAGGGATCAAACAGGAGAAGGAATCTCACTAAATGCCAACCAACTCTTGTGGGAAATAATTCATTCCCAGAGAACTAA
TCCAGTCTCAGGAGAGCAAGAACTCACTACTGTTAATACAAGTGTTCAGTTACCTCTAGAACAGCTGCAAGCCATTCATGAGAGCTC
TGCCCCCATGACCCAAACACCTCCCACTAAGCCCCACCTTCTGACACCACCACATTGGGGATCAAATTTCAACATTCATTTTGGTGA
GGGCAGACAAACCGTGCTCAAACCATAGCAGAATGCTTTTGGCTGCATGTAACAGAATGCCCCCAAAGTAGCTTTACCTAAGATGAG
AGGCGCTTACTTAAAATAGGAGCCCAGGGGATTGCATGGAACCCAGCATAAGGAGTGCAACCACAGGTCCCGGGATAAGAAGCTGGA
AGCAGTCAGGAACATAAGCTACTCTGGGTCTCTTTTCTACTTTCTATATCTACATAATATCCCCCAGTCTATTCTAGCATGAGGGAG
ACTAGGCTGAGAAAAATCACTTTGTCCAATACCAGACTCTTGGGAAATGGGAGACTCTGGCCCAGCCTGGTATTCAGTGGAACAGCT
CTTCCTGAGTCATATGTCTGACCCTAGTGACTCATCTGTCCAGAGAGTGGGAGAAGGCCCATCCTCCACAAATATGCGTACAGGAGC
CCCTGTTGGGGCAGGGCCAGTTTTCAGAAAGGGGGATGTAGATGGAGGAGCTAACTCGATTAATATTGGTTATCATTTGTATCCCAC
GTTCTTCTGAAAGGATTCTGTAAAAAAATAGGCACAATGAAACCAAACCCATTCACACAGGATATCATGCAAAAACCAGGAGCAATA
AGGTTACGGAGAGGAAAGATGCATCTAGAAGCCCTGATGGAGAGAAACTTCTTTAATGCAGAAATTAAATTAATCAAAATTAGTGGA
TTTAAAAGTTCTGGGGCTAGTCATCCAGAATTCATGAACTAGGTTGCTAAGAAAAGCAATGGAAAAGATGTGAAATTAAGTACCATA
TGAGTATCTGACTAGAGTGTGTTAGTTTGTTTTAAATGCATGGGATACTGTATACATTTTTTCCTGGAATGACCCTCAAGCTGAAAT
TGAGGAGAGAAGATAAATGGGAGGCCGGATGCTGCCACTTCCTGTGAGCACATGCTCTCTGGTTAGGACAGGCCCCATCACTGATCT
CTCTTATCTAATCCCAGGTCACATCACTCCTCCATAATCCTCTGTGTTTGGGTGTGTGTGTGTGTAAATGTCAAATAAGGGCACATG
CAGCAAGTGATACAGAAGTTCAGGAGAGGGAGAGAGAGAGAGAGAGAGAGAGCCTCTGATCACTGTGAACTAGTATATTTTCTTGTA
TTTGGAGGAGGCAGGACGTGAAGCTGCATTTCATTTTGACCAGTTGTAGATGGCAGGGTTGGAGGGGCAGGTTGAGAAATGTGTCAG
GAGGGGGGCAGGAACTGATATTAATGACAATACTTGCCAAGCACTCAGTGCTCACGTGGTCACATCAACACACACAATTGCTTTGTG
CAGTTCTGAGGTGCCCTTCTTATCTGCATGTGACAGGTAAGGACACCAGAGCTCTAAGAAGTCAAGAAACCCTCCAGTGATTCTTTG
TCTCAGGAGAAACCAGAGTCCTCACCACAGCCTGCAAAGCCCAACACAACCTGGCTCCTATCATCCTCTGTGCTGTGTCTTGCAGCC
ACTTGGTGGCTGTGCCTCCAGACTTTCCCTGGAATGCTGTTCCCCCGACACCTGCCATTCACTCCTTCACCTCTTTTGTGTTTTTGC
TTATATGTCACTTTCTTAGTGAAGACCTCTGTTATTTGCTCTGCTTTCATATTGCAGTGCCTCCTCTGTCTTCTGCCACATGACAAG
CCCGTGACACTGATCTCTTTCCACAGCACTTCCCATTTTAAAAAATTCCCTGCATCTACTCACTTATGTTTCTTTATATTGCCTTTC
TCTTCCTGCACACTTCAACTAGAATCTAAACCCCACAAGACCAGTCACTTTGTTTTGTTTCCCACTTTATCCAAAGCCCCTAGATGA
CCTCCTTGCTCATGGTAGGTGCTCAATAAATATTTGCCAAATGAATGAAGACCCTTTCCCAAGATCATGAAACTACGTAGCACTGGA
GTCAGGGTTCAGCAGGCAGAGTGCACTCGGGCCACACTGAAAGGAGGCCATCAGGATGAAGTGACATGAGAGATGGTGACCCTGGCT
GGATGGGACATGTCCTGTTTACAGTTTGAGCTTTATCAGTCACGCCATCTTACCATGAGGACTCCGGACAGTTTGAATGTACAAACT
GGGACACGGGGACCCAATGGGAAAACTGGGATTGACTTTCTGCATATTGAATTTTCCACCAGCCCACAGTTACTCCTTTGACATGGC
CAGAATTATCCCCAGAGGTTTTTTTATTCCTGAAATAAGTCCCTCCTCTATAAAGACATTTATTTTGTTATTATAAGTATTTAGCAC
ATTTGTGTGGACCCTGCTAAAACGAGTGTTGTTCAGACTAAAGTGTCAGAAAATATATAATACAGAGTACAGGTGCTGCAGCTTCAG
GAAAATACAACTTTCCTAATGGCACTTCTCCCCTTGCCTCCCTTCTATGCTAAATTTTTAAATGCATTTCTCCTGTTTATTCAGGCA
GGGATTAACATTTTCTTAAAGACTTCATCAGAATGTTGGGTTTTTTTCCATTCATTGTTATTTAGGATGATATTTTTGTGACTCCAA
TTTAGGAAAGAAGATTGACTTGATCTTTATTTTCCTCTATCATATTTACCTTGTGTGCTGTTATTTTGAGTCTGTTCACAGGATCCT
GGGCAGATGCCATCAATGTAATTGCTATGTTTTTAAGCAAAGAGGATTGAAAGTGTCAATTTAAGAATTGTCTAGGAACTTGATATA
TACTATCTATCTATCTATCTATCTATCTATCTATCTATCTATCTATCTATTTGCTTTTTATGAATAAAAGTAGCAGGGGTGTTGGAA
ATATATGTATATTTTTAAATAATTTATTTTTTAAAAGTATACAAACCGAAATTTATTTCAAAGAGAATTCAGATAGCAGCCAAAATA
CACAGAAAAGAAAACTGTCCTTCAGAAGTGCCACTAGATCAAGCTAGTTATGAAATTGGCACCTGAGAATAACTTCTGTCAGAGGGG
ATGTAAGTCTTTGGAATTTTGAAGAGCCATAGTCTCTGTAGAAGATGCCAGAAATGTAGGCATTATCCTTAACACTTCCTTTTCCTT
CCCCTTCCTCCCACCCCCACTGAAAATGTCAGTTTTACTTCTCAGTAGCTCTCAATTCCGACCGTTTCACCGTATTTCAGCCCAGGA
CATGATCACATCTTGCCTGTATTACCAAAGTCACCTCCTTTTGATCACTGTAGATGCACTCAGACTCTAATCTTTTCTGCGGCCCAG
ATCCTCATTTTTCCAAAGCCCAGATCTGCAAAGACCTGCCTGCCTACCCCACCCCTGCTCACCACACATAGCTGGCCTTTAAATGGC
TTCCTGGCACTTGTAGGAGAATGACTGGATTCCTTAAGGTGGGCTACAAGGCCTGCATGGGCTGGTCCATGCTCGTGTCTCTAATTG
CAAACCTGCTGTACCTTCCCCACTTCCCCCATTCTCTGTGCACCAGCCACAGCAATTGCCTTTTAGACCTGTTTACTTGTCATGCTT
CTCTCTCTACTGGGACTTTGTACATTCTGCTCTGCCTGAAACACTCTTCCTCCTTTCTTTGCTCATTAACTCCTTCTCATCCTTTGG
ACTACATGTACGCATTTCTCACTCAGGGAAACCTTCAGATGCCCCTCCAGGTCAAATCCCCCTGTTATAGTTTCTTTGGCCTTATTT
GTTTCTCCTTTGTAGCACCATTAGGAATGATTTTACGTTTTACTCTTTATTTGAGGACTCTCTCTCTCTCTTACTGGACTGTATGCT
CATTGGAGGCAGGAACTGTTTCTGTTTTCTTACCTTCATTTATCCCAAGAGAAAAGTAGTGTCTAGTACAAAATAGGAACTCAAGAA
TATTTGTTGCAGATTATAAATGAATGGATGAATGGCCTCTTGAAGTATCCATTTTTATACCACTTAGAATAAGGCACTGTACCAATT
TCAAAATGTTGGAGGTTCAGCAAGGAAGAAAACCACCAGATAGGGAGGAAAGGATCTTCCTTTTGTAGAATTCAGAGAAAAGTGGGG
CAGATGTGTCCCTCCAGAGGTAACTAATCGCCCTCATCACATGCTGGGGCCATTTTCTTCTCCAGCTGTTCCCAAGGGTCAGCTCTG
CCGGCTGTCCTTCTGCTATGCCTGAAGGCTTCCATCAGCCTGCCTGAGGGCTGTTCTCATTCCTAGGTGGGAAAACATACCTTTGAT
GCCTAGAAATTAAAAGAGTAATTTAGAGACTTAGGGCATGAGTTTCATTACAGTTCTCCGTCCCTGAGCTTGCTCTGTGGACAAACA
TTCTGTGCATATTAGGAAAGTATAATAGTTTTGGTCTTGGGCTAAGGATTCAGATTTTCTGAGCAATGTAGCTCAGGCACCTTTATG
ATCTTGGGAGGAATACTTAAAATTTCTTTTTTTTTTCTTTTTTGTTTGAGACGGAGTCTTGCTCTGTCGCCAGGCTGTAGTGCAGTG
GCGCGATCTCAGCTCACTGCAACCTCCGCCTCCCAGGTTCAAGCGATTCTCCTGCCTCAGCCTCCCAAGTAGCTGGGACTACAGGTG
TGCACCACCACGTCCAGCTAATTTTTGTATTTTTAGTAGAGACGGGGTTTTACCATGTTGCCCAGGATGGTCTCGATCTCTTGACCT
CATGATCCGCCCACCTTGGCCTCCCAAAGTGCTGGGATTACAGGTGTGAACCCCTGCACCTGGCCTTAAAATTTCTAAGCCTCAGTT
TCCTTATCTATAAAATGGGGATTAAAACAATATCTAGTGCCTGGGATATTGTTATGGGATACTTTGTACCAACATTATTCTATAAAA
ATATAATGCAAACCACCTATGTAGTTTACGTTTTCCATAGCTATCTTTAAAAAGTGAAATGAAATTAATTTAAATATTTGACCCAGT
ATATCCAAAATGTTATCACGTCAAGATGTAATCGATAATAAATATTAATGAGATATTTTACATTCCTTTTATTCATATTAAGTTTTC
AAAATTCAGCCTGCACTTTACACTTACCAGTGCAGCTCAGTTTGAAACAGTAGTCATGTGCCATATAATGATGTTTCAGTTAACAGC
CAACTGCATATACAATGGTGGTCCCATAAGATTATTATATTGTGTTTTTACTATGCCTTTTCTATGTTTAGATACGTTTGGATGCCA
GATACTTACCATTGTATTACAGTTGCCTACAGTATTCAGTACAGTGACATGCAGTATAGGTTTGTAGCCTAGGAGCAATAGGCTATA
CCATATAGCCTAGGTGCGTCATAGGCTGTACCATCTAGGCTTGTGTAAGTACACTATGATGTTTGCATGACTACACATTTCTCAGAA
CGTATTCCCTTTATTAAGCAACACGTGGCTGTAGTTTAGCCATACTTAGCATTCAACAAATGCCGGCAAAATTATTGTTATTTATTT
TAAAACATCAAAATTTGGGTTGACAAACATTTGTACTGTACATTTGCATATATTGCCATGTGGGAAGATGCCTCAGATAAGATGCCT
CAGATATATTTTTTTCCATTTCAAAAGAGTAAAGGGTTGACTTTAAGACTTCTCAGCTATTTCCTGCAAGTGGGCCTCCCTGGGTTT
CAGAATGAGGTCAGTTTCGTGGTTTATGGTTGTCTTTTCTTTGGGAGGTGGGAAGGGGAAACTGAATGTGCCTCAAGTATTGCATGC
CTGTCTCTGTTTTTTGGCTCTTCTCTGTTTAATTATTGGGAACTGGAACAACTTGATTTTAAAACAAAACATATGGTCTGAGAATAA
TTGGGAGGACATTTGCTAGTGATGTAGCACTGAAGGTATGTATCAAGTTGAGGCCCAGGCTTGCACCACTTGCTTGCTTAGGTATTG
AGTGTTTCCCTTAACTTCAAATTGTGTTCTTTCCCAGTTCTTAAACCCTTGTTTGGAAAGTCACAGTCAAAGCAGGAAATATGGGAG
CAAGATTATAAAGGAAATTGTCTGTGAGTCTTCTTTAAATCCCTACTTTTTTTCTCTCTCCTCTTTATGGAAAACCATTGGTTTCCA
AGCTATTTATATGGCTCGTGAATGAAAAAAGTTGTCTCAAAGTGCCAAGAGGAGAGACGAAAACTTTATTTAAAAAGGAGAATGTGG
ACCCACCATTGAATTTCTCTGTTCTTTTGCTTAACTCCATTGTGGACACCAAAGTGGGGAATTTCTGTTCCACAGCCAGTTCTTATG
TGAAGGCAAGGTTTTTATCTCTCTTTACCTTTTCCACACCCCTTTGCTTGCTGGGGGAGGAATATATGATATGCTGATCATGTACCA
TGTAATTCCAGCCTGAATGCCTCAACTTTTTATATCATGGGAGCAAAGGGGAATTTATCAGGACTGCTAAGAACATGTATTTCTCTT
GTTCAAGTCTCTGCTCCTCTGCTTAGAAGCTATGTGCCTCTGAGCAAGTTATTTAAACTCTCTAAACCTCATTTTCCTCTTTCCTAA
AAAGGGGATAATAAGAGCGCCTATTTCAGAGTAAAATTGTTGTGAAGCTTTAAAGAGACATTGCACACAAAGCACTAAGCATGATGC
CTGGAAAGTAATAAAAACAAATCTTAGCGATCAAGCAGAGGCCTTCGGCAGGAACATGTCCACACCATCAGCAGCAGGAGCGACAGT
GAATACTGCGTCAGGGAAAATAACCAAACATTTGTCCATGAGGAGCTTCAGCATTGGAAAGTCAGCCATTTCACTGGGTTGTGTTTG
ATGTCAATGGGTTGAAGTCAAAGCCTTAACTCTGTGCTTCTGTGCTTCCTAGGTCTCATGGGAGCCTGTACACTCACTTGTCAGTGG
TCGTTCTCTTTTGCCCAGCACCTATAAATATGAGAGTAACATGCTGAATTGTTTTACTTTTGTGCTACAGAGTCCTAAGTACCTTTG
AGTCTTTTGGAGATTCACTTGTTTTGGATCTGAAATGTAAAATGAAATAGCTCTCTGACCAGGGCTGAGCACGGTTTTTCAGTTGGG
ATAGTAAAACGAGAAATCTTTCTGAACACTTTCCAAGGAGTCCCCTATAAGAGCAAGGGGTAGGAAGAAAGGGGTCTTCAGTTCTTG
CCAAATCAAGGACATGATTTTCTGTATAGGGCTCAGAATGTATTGTGTGTTGTCTTGCTTCAATGTCAGATGAGTGTCGAGAGCTTC
GGGAAGGTCTGCTTTTTAAAAACACCACTATGTTTTCGGAGCAGCTAATTGTCTTTTGTCAGTCCCCTTTTCTTTCTGCTCCTGCCA
TCTGTCCAAGTCCCGTTTGGATTTATTAGTGATTGCTTACAAAGTATCAGGAACTCCCTGCTGAGAACAGTGAAGCCCTTGCCCAGT
GGGCAGGTCTCGGATTTCCCTGGAACTGTAAAAAATAACCACTCTCACTCATTTTTTTCTCTGCATACCCCTCTCAGTGGACCACTT
TCCTCACATTAACTTAATCAAGATGTTAGTAAAATAAACGGTGTGCTGCCATCACACTGAAACTTCTTGTCTCTTTTCAGTGTTTGG
AAAGGAAATTGAGAGTTGCTATTGATAATAAAGATGAAGCATATCTTTACTCATGGCCAATTACACACAGTCCTGATCATTGGATTT
TTAGTGGGTGGACAGCTCTGTACAATGTGTCATCAAGTCTGTGTCGCCCCAGTTACAAAACACTCCATTGTTTTCTATATCAAGGGG
GAAAAAATTATGAGATACCATGGATTTTAAGATACATCCCAATTTCAGATGTGTTCAAATGTGGGGAAAATATATGTATCTTATAGC
AGATGGAATACAGTGAGAATTAATTAAAATGGAAAGCCCTAGTCCGGGAGTGGTGGCTCACACCTATAATCTCAGCATTTTGAGAGG
CCGAGGCGGGAGGATTGCTTGAGCCCAGGAGTGTGAGACTAGCCTGGGCAATATAGTGAGGCCTTGTCTCTACAAATAATTGAAAAA
TTAGCCAGGCATGGTGGCATGCCCCTATGGTCCCAGCTACTTGGAAGGCTGAGGCAGGAGGCTCGGTTAAGCCTGGGTAGTTGAGGC
TGCAGTGAACTGTGATTGAGCCACTGCACTCCAGCCTGTGTGAGAGAGTAAACCCCATCTCAAAAAAAAAAAAAAAAAAAAAGAAAA
ACAGTAAAATGGAAAGCCCTCCTGGTTATCACCTGTTCCTCTCCTGGCATTCCCCCAGTCCTCAACACAGCTAATTGACAGAACGGT
GAGATTTGTGGGTATGTATGAGATGTGCCTCTGCCTTAGGGCTGAATGCGTAACTCAGTCCCATGTGTTGTATACTCTGTGTATGCA
TGAAGTTGTGGAACTGAGTCAAAACCAAGAAGGGTGGTGGGAGGTCCTCTGAACTAAAATTTACACAATCGAAGTTTCTGAAAACAG
AACTTAGGAATTTTCTTTGGGTTACATTGATAAGATTCTTGTTACCTTTGGAAGGCATGATCAAAGAGGGACATCCAAGGCTCTACA
TGCCCCTCGCTGACCTGGCCCATTTGTAATGGCTCAATACTGAAGAGAAGATCCCAGTTCCTCTAAGTTAATGCCTCTGCTTATCCA
TTTTCTAGTGACCACAGGCCTCAGATCCATGGGGAATCCGTTTCCTCCCCTACCCTAAAAGGCAGCATATTAAATGTTATTGATAAT
TTCAGTGTACTCAGGATTCTACCTAATTATTACTCTTCATAATCTGAATGACTTCTAACCAACTGTGTGTCTCCAGCCCTTCTTAAT
GCTGTAAAAATTTTAAGTAGGGGGATTATGGTATTTATTTTCCTGGTAAAGATAAGTGCTCACACTTTAGGAAAAGCTATTTCCCAG
CCAGGTAAAGTGACAAAAGAATTGATTGAAAAGTTTCTTCTTTATATTAGCGGCATTTTCCCTTATATTTACACTGCTTTCTTTCTT
TTTCAGTTTTTGGGAATAACATTTCTTGGAATTGGACTGTGGGCATGGAATGAAAAAGTAAGTTGAATATCATGACAATGCACACAT
CGTGGTTGTTAGAGGCTGCACATCATATTATGTATTATCCCCCTTTCTCTTTGACAACCACAGAGTATAAACATGGCTCTTTAACAA
GAAAAAGTGTTGGTGGAAGATTAATGTCATACTCTCAAAGACACTTTAGGAATTGTGCCTAAATTGATGACTGCATCAAAATAATTA
CTGAAGTAACAATACACGTGCTCAACAGAGAATATACCTTGGAATTTGATCAGTTAGCCAAAGATCGATGTTCCTATGGGATATTTT
CTCACTCAGGCACAAGAATTCCAGTAATGATTATAAATATCACTAAACCACCTGTAGCCTTTTTTTCTCTTCTGCTTTTTTTCTTCA
ACTCTTTGGAAGATAACTCAAGATAAAAACGATTTTTTTTAGTGTGTTATGTGGTAAACAACTATTAGCATGTGGATTGAAGGAAGT
GGGATATAACTTCATGTTTGAGTATCAAATACACTCATCATTAATGTAAATAATGGGAATGTTTGGCTGCTGGTGGCAAGTATTAAG
AAATATTCAGTTTATGATCAAATACAGTATAATAAAAGGAAAATCTGGAAATACAGAGTAATCTATGTGACAGTGAAGATACAATGT
TCTGGGCAGTTTCTGCTCTAAGCAACGCACTCATGCAGACACCCCCTGTGTCCACACCTCTCCTTCCCATCGCACCTTCTAATTTTT
CTCTACAGCTCCCCCTACTGAGGCCACCAGTGGAATTCAACAGTGAATATAAACACCCGCCCCCTCCTCCCAGCTTTTTTCCTTCCT
TCTTTGCAAGTGAAATCACCTGCAGCATTTCATCTATCCTTGTATCTGGCTATACACAAAAGCTTTATTATCTCCCACGTAAGGACA
AGGGGATGTATAGAATGGTTAAAAGCTGCCATAAACTAAAATCTAGCTTAAAATTTCCCAGAAGACCATTCTTTGATCCCGTAGGTG
TTTCACCCTCTAGCCTGACCCTGGCCTGAAGCTGAGAGTTCCTTTGAAGTGGTAAATGCATTAAGAAAGGAGAGAAAAACTGGGAAG
GTCCAGTCAACAAAAGCCCCTCAGAGGCCAGTAGGCATTTCCACAACTAAAACAATGTTCAGACAGACCATATAAAACAAAACTCAA
AGCAAATCCTGAGTTTTGCAAATGAACCATCCTGTATTTATTTACAAAATTCTTCTACAGAATGTAAAATGTTTTATGGAAAACAAA
CTACTTTTTAGCAACAAGAGTAACTGAGGCTACCTGGAGTTTTGAGGCAGGGCAGTGATTATGTATGAGGGGAGACAAATTAAGTTC
CCAAAACAGAGATAATTTCATCTCTAGAAACTTTTACATTGAATATCGGTTGATCTTGTCTTGGATTATGGTTAACTTTTGCCTCAA
GCCTATTGTGAACTTGGAAATTTGTGGAATCTGGGAAGTTGGTTAGATGCCAGATAACACAGCTTTTTATAACTCCTTTAATAAGAG
TCCATTTCTATCTGCTTTGAAAAAATGCTGGAAGGCATTAAATGAACTAATTATCTGTTCTAGCAATAAACAGCAGTTGGTAATTTT
GAAGTTTTTTAATTTTCTCTTTATGAAATATAATTATCTTTTTGTTTCATCAGTTGCCTCAAATGTCTCTTCTCTGATAAAATCACA
AACTTCTAAAACTGGCAGTGTCATACAAATATCCGAGCAAGGGGTTGATGCGGTCTTTAGGGAGGGATGAAATAGCAGTTCCTTTTT
TGTAAGGGCCACCATCAAGATTTCTCTTGAGATCCGAAGTTAACCTGGCCAAGGTCACCCAGCTCCATGTAGCAGAACTCCATAAGC
AGGGAACAAATAGAGGCAGCAGAGGGTGAAGCGGGTGTTAGCCGTGCACCACCCCAGTTTGGTGAGTGCTGGCCATGTGTCTGGGGC
CTGAGTGTGGGGAATACGACAATGAGGGATGCAGAGTCCATGCCTCATGGAGCTTGCATTCTGGTGGGTCACCAATGAGAAGCAGTT
ACTGCCCAAGTATGTTTGTCCAGATACTGGTAAGTTCTGGCAGGAAACATCAAGTTAGAGAGTGGTGGGAAAAGGAGTCTCTTTTAG
ATGAAGTAGTTGCCAACAGCCTCTCTAAGGAGGTGGCATGGGAGCAGAGACCTGGATGAAGGAAGAAGGATCTGCAGGGTATGGGGG
AAGAACATTCTAGGAAGAGGGAAGAGCAAGTGCAGTGGCCCTGAGGCAGGGACTTTGGTGACTCCAGGGAACAACAAGAAGGCCAGT
AGGCCTAGAAGGGAGAAAGCAAGGACAAGAAGAGGTGGGTTGTGTTGCCAGGTAGAGGCCAGACTGGGCAGAGTCCTGCAAGAAGTT
TGGATATATTCTAGGTACAAGGGGAAGCCACTGGAGCATTTTGAAAAGTCTAATATGACTTTTTTTTTAAGATCACTCTGGTTGCTG
GGTGGGAAAAGAGCACAGCTTTGTGGCTTTGCTATGGACTGAATATTTGTGTCTCCCCACCCTGCCAAATTCATATGCTGAGGCCCT
CATCCCCAATGTAATGATATTTGGAGGTGAAGCTTTGGCGGGGAGGTAATTAGGTCATGAGGGCANNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
CACCCAGTTTATGATCAGTGTTATACAGCCGACTAAGACAGGGGTAAACAGACCTGAGTTCAAATACTGGCTCCTCCAGTATTTGAC
ATGTGTGGAATACACACATCTCATCTGTGTGATGTTGGCCAACTGAGTTAACTTCTCTGAGATGCCATTTCCTTGTTTGGCAATAGG
TATACTGTTCTCACCTTATAGAATTGTTGTGAAGATTAAATGAGACCTCCCTCAGCACAGTGTCTGCTCTAAGGCAGATGCCCACTG
GAGACATGGCTCCCTCCCCTGGACACATGACCATAGGCTGTCCACTCTTAATATGGGGCAAGAGCACAAAGGTAGTTTTGAAAGGCA
GCCTCAAATTTTAGGAGACTTTCAGGCTCCAATCCATGGACCAGAGAATTGGGTTTGTTTTGTGGTCTCTTGAGGAGGATGTGAAGT
GTGTTGATCTCCTTGGTAACTGCAGAGACTGCTCTCTCAAAGCCCTTTGGGTTAATTTCTGAGTTCGGCCAGGCCTCTGCTGGAGTA
AGTTTTCAAGGTGAACCCCAGCCTGGTAGACAGTGTGGTGAACACTGCACCTGCTGAGCTGACACCATCAGCCCATGAATCAGTACA
TCCCCTAGACCCAGATGTGGCGACATTTACTTGTAAGAGGCCTGGAGGGTAAATCCCCAAACTGGATCATAGCTGGAAGGATGAGAT
TTCCACTGGAAGAAACACATTTTCTTAGCAGATAGGACTTTCAGCATATTCATGTGAAGTACAGAAGCATTGAAAGCAAAAATGATT
ACCCCATACTTAATATCAGTCTGAGTTTTAAACCTAGGGCTAATATTGCAGTTCAACATTAGACATAATAATTTAAAGAATTCATTT
TGTCTGAGTTTTAAATCAACTGTAAAATGCAAAGGATGAGTGTCTCCAAGGGAAGCTGATTTATGTGCAATTGTTCAGAGATCATAA
GGATCTGCTGTTTGGAAGGAACATTAACAATTATGTGACATGAATCACCTCCACAACACCCTTGACAACATCCAGTGACAGTCAACT
CATTCATTCAGATAACATTTATTGAGCACATAGCATGGGCAGGGCTTGTGCTGAATTCTAGGGAAATGTGGATGAAGTGACCTAGTT
TCAGTCTTCATGGAGTTTATCCAGTTGTAGGAAGGGTGTGTGCATATACCTAAAATAATACACTGCAATAAATGCTACTTAGGGGTG
TATATATGTTTAATGGTTCCCACAGCAAAGTGCTCAGCATTTCTGTCTCTTTAGGTTGCCCTTCCTGTTGGAAAGACCTTCATTCCA
CCGAATTAGAATCATGTCCCACTAGTTTTCACTCATTGATTCTGGATCTACCCTGTTGGATCCCACAGAATAAGTCTTTCCACATGT
TAGCCCGGGAGGTATGTCAGGGTGAGGGCCTGGTTCCTTCTGAGTTTCCAGGCTAAACATCTGCTTCTCTCTAGGTGGTCCTCACAG
GATGTGGGCTGGAGCCCCTTGTCCATTTTGTTGGTGCTCATCTGGCCCCGCTCTGGTGTCTTTTCTGCACCATGGAGAGGAAGCTTT
AGGGGCTGTAGAGAGTGTGGCTGGATTGCGTCCTTTCTCCCCTCATCAATCTAGCTTGGGATCCACTGTAGATCACGGTGTCTGCAG
CTCACTGAGAGTCCCAAGTAGGTTGCAGACCCAACTGGTTACCCCATCCACCTCACCTATGGCTGATTTTCATAGGCAGTTGCAGGA
TTCCACAATGACACAGTGATATTTCAGCCCACCTGTCCACCTGCTGAGATCACTCCAGAGCCTCTATCTAGCATCTTCCCCATTCCG
CCCAGCTTGATTTCAGCTATACATTTTGATCAACCTCCTCCCTCTATTCCTTCTGCTAAGGCAGACTGTCCTTTTGCCCCACCATGC
CACCTGGGAAAATTCTGATTCAGTAGGTCCAGGATAGACACTGCGATGCTCTATTTTAACAGGCCTACTAGATTGTTGTGACTGCAG
TCAAGTTTAGTTATCATTGATAAAAGCCAGTGACATGCTGTTGAGCAGGCCAAGGACAGAGCCCAATGACATGTCACTAGAAATTTC
CTTTGGGGTTGACACTCATTCACTTATGTGTGCCCTTGGGTGTCATCAGTCAAGGAGTTTCTCATCCATCTACTTGTCATTACATCA
GACTATATTTCTGCATCTTGTCTTTGTAGATAGATGGTATCTTTCACTTTTTCACTGGTTAACTGGCCTAGGCACTTAACTGAACCT
AGCACTTTCTTATTGAACCCATGCTGGGTCCTGAAGCACATCATTTTCTCATCCAGGTACTTGGGAACCTTTATCAGCAAAGCAAGG
ACTAAGGCATAATACATCACTCCTCAATTCGAGAAACGTTTTCCTCACTTGGCTTCCAGGATATCACACTCTCCTGGTTTCCATACC
TCACAGGTGGTGCCTTCTCACATGTGTTTGCTAGTTCCACCTCATTTCCCTGACCTCTAAATTTTGGAGTGCCCAAGGCTCAGTCTG
AGCTTTGACTCGTCTTAAGAAATCTCATCTAGACTTGGGGCTTTACATGCCATCTGTCACTGATGACTTCCAAATGAATATTTTTGC
TTCCAACCTCATCTGAATTCTAGACTTGCATAGCCAGCATCCACACTGAAATGCCTGGTAGGCTTCTGAAACTTAACACATTCAAAA
CAACACAAGTGATTCTCATGCCCTCCCAACCTGCTCTTCCCAATTGCGGTAAAGAAACAACTCCACTCTTCTACTTGCTCATTCCTG
ACACTCTGCTTTCTTTTCTCCTCATACCATATTCAGCCCATCAGCAAATACTGTCAGCTCTACTTTGAAACTATATTGAAAATCCGA
TTTCTTCCACCACCTCCGCTGTTATACCCTGTTCTAAGTGGCCACCACATCTTGCCTGCATTTGTGCAGAAGCCTCCAATTATCTCC
CTGCCTCTGCTCTTGCACCCTGACCCTCACCCTAGGGCTGTCTCTGTGCAGCAGCTGGAGTGATCCTTTGGAAATGTAAGACAGATC
CTTTTACTTCTCTGCTTCTAGCCCTCCAGTGAGCTTATCTTCTCACTCAAAGCCATATATGGTGCCCATGGCCTGTGTGACATTTGC
TTTCATACTGTGTGCTTCCTCACTGTGTCCTATGGTCCTACTTATCAACTTGCTATTCCTCCCGCATCCCAAACATACCCCCACAAC
CCCAGGGCCTTTGCACTTGCCCCTGACTGGCATGCTCTTCCCCAGAAAACCACATGAGTGCTATCTTCTACTTCCTGGTTTCTTCTC
AAATGTCACCTTTGACCTAGCCCTTTCTTCCCCTTCCTTGTGTTTTTCCTTCTCATGCGACTCATCACTTAGACGCTAACAAGTTTA
TTGCCTGGACTTTTTATTTATTACCACACTCTAGTACCTAGAATGTTAGATGGCACATAGCAAGTATTCAAGAGGTATTGCTTATGA
AAGAATGAATGAGCAAATGAGCAATGACAGCATTCTTTAAAACACTGGCTATGTCATGGAGTTATAGAAGTCAGTACTTGCCTCACA
TCTCCAGTAAGCATACTATATTAGCGATTCTTACAGAATAAAAGGAGGTAAATACGTGTGGTTTCTGAAGTCTGATTTGAGAATAAT
ACGGGGTTAGAAACAAAATATATATATTGTCTATATCAGAAAAGAAGAAAATTACTTCTCTCGTAAAAGTGGAAATCTATATATGAT
TATCTCATTTCATGGAAAAATTAGGGATGCCATTCCTTTTCCGTTTTGCTCAACTCTTATGCTTATTACTTAGTTCTTAAGTGGGAG
CAAGAGGGAGAGTGATCACTCACAATTAGAAATAAAGGTATTGGGGCGAGGGAGGACTCAGCTTTTCAGAGGAGGGAAAATGCCTTT
CCCTGGTCCTTAGTGTGAATTGAATGTTCCCATTCTGCCCCTGCCTCCCTAATACAGCACCTAGCCCACTAAGCAGAGTGGATACCT
ACTCCTGGCTTGTCTGGCCTGGAAACTCCTTGAGGACAGGAGCCTGTCTTTGCCCTTTATTCTCAGTTCCTACCACAATGCCTCCTA
CACAATGGATGCCCAGAAGCTGTTTGTGACTAAGCCAATGAGAACATGCAAAAAGAGCTTTACACGTTGTAAAATGCTACACAGGAA
AAAAATACCACTTTTTATAAAGGAGTTGCATTTGCACTTCCCTGACTTCTGGATCCCCTCAAGTTAACTTTAAGAGAATGACATTCT
ATTTCATAGGTGGGGAAACCAAGGCACAGAGATGTCAAACTGGAAGTCTGGCCCTCTCCCTCCCAGTCCTCTGGCCAGTCCCCTGTC
ATCCTCCATTGCACTCACTACGCTGGGTTCTCTAGGGGTGATTGGAAGGAAAGAACTGGTGCCCAGTGCTGATCCCAGTAGGGAGAA
GGGGGAATTAGAGGAGCTCTACCCCCCATATTGCTTTTCTCCTTGGCTTTGCTGTAGATGATGTTTCTTTTTCCTGGAGGGTTTTTC
CCAGCCCACTGTCTGTGATTTTAGGTCTCAATGCAGAACACTCCAGTTCTTCCTGTCACAATTATACAGGAGACTCACCTTGGAAAA
CCTTTCCCTATTTGCCGAGGAAGGAAAATTCAGTGAGATTCATTGATTCAGGAATGTCATAGCAGTACAGGCCAGTTTCTGTTGCCT
GAGGGAGGCAATAGAGGAAAATGGTTACTCAGATGACTTGAGCAGAAAAAAATAGATTTCTTAACATTGAAACTTCTTTAAGGAGAC
ATTACTGAGATGCTGAAAATACTTCTATGAAGACTTGGTTTTATTGACTGAAAGTAGATTGTGATTCTTTTCCTCAATTATCCCAAT
GATTCCCTCAAGTTCTCTGTTGTATCTAAAGAGCTGGAAACCGGCCGGGCGCGGTGGCTCACACCTGTAATCCCAGCACTTTGGGAG
GCCGAGGCGAGCAGATCACGAGGTCAGGAGATCGAAACCATGCTGGCTAACACGGTGAAACCCCATCTCTACTAAAAATACAAAAAA
TCAGCCAGGCATGGTGGCAGGCGCCTGTAGTCCCAGCTACTTGGGAGGCTGAGGCAGGAGAATGGTATGAACCCAGGAGGCGGAGCT
TGCAGTGAGCCCAGATCACGCCACTGCACTCCAGCCTGGGCAGCAGAGTGAGACTCCATCTLAAAAAAAAAAAAAAAAAAAAAGAGC
TGGAAACCAAACAGCTCATGGTCAGTACCTGGAAGCTATGTGATCTGATGTGCTCTAATCAGTAATGTTTCACACACACATGAAGTA
TATGGGAAGAAAATTTAATGTTTCTCTATGTATTCCCCTTTAAACTGTATTCAGTGGGGTTTCTTAGGGATGAAAATTTAGTTGGGT
CAAAAATTCAATGACTTTGTTGTTCACTCTCAGATATCACAGTCCTTCTCTCACTTTGAGGCACAGATCCAGGCCCAGTGATTAGAC
AGGGCTGGATAATAAAACTGCAAACCTAACTTTGCATTAAAATGTTCTCCCTGCCGAGCCCATCAGCCTTGGCCCCTGAGGAGGGAA
GGGAACATGGGCCCTTGCCCTCTGCATCCTCCTTCTGGCCCCTGGCCCTGCCCAGATGCCTCTGCCCACCGCCTGCCCGGGCAGAAG
CTGGGGTGGGCTGTGCTCTCTGGGCCTGGTGGAAGTTAAAGAGGAGAATATCCTGGTCTTTGTGAAGCTCCCCTGCTGAGTCCCTGT
GCCTAGTGATTTCCTTGAGCTTGGTAGCCATCTCAATTCCAGGTGACCACCTGGCAGCCCTGCTCATCCCTGGCCACCTTCCTCAGC
CAGTGCACCTCTGAGCTCTGCCCTCTGAGGTCACTTCTTCACACGCCTGCAGGCAGCCCTATCTGACAGCGTCCAGGATGACCTCAG
GCCACCTCTTCCGTGCTGTCCCCGTGTTTTCTCCAGGACATACTCATCTGGGAGCGAGGTCAGTGCCCCCAGAACAGCCCATTGCCT
GTCCTCACCTACTTCTGATACCAGAGCAGCCAGCCGGCCCTTCTCTCTCTAGGTTGCAAGGGCAGGAGGCAGCCCACAGCCCCTTCC
TAGGCAGAGTCGGGCCCTAATGGCTGTGCGGGTCAAGTGCAGAGAACCACTTCAGAGCTCCCGGGAGGTGCCCTTGGAACTCCTCCT
AAGGTTTAGGGTGAGAAAGAGGCTATCCACACTCCACCCCCAGGCCCTGGGCAGTGGTGTCACAGCACAGCAACTGCTTTCTCCAGA
GAAACCTTGCCTCTCAGTCCTCCTCTGACCCCTTTTGTCCCTGCCAGATTTCACCGAGGCGCACAAAAGAAAGATGTCCTTTGTCGG
CGGTGGTGGAGGTGGGGTGTGGTTGGGAGGTCTTGTCTCCTACTCACTTTGGTTTTCAGATAGTGATCAGTGAAATCAGAGGCAAGA
ATGTTATAACATATTGTGTATATTTTTCTTAAAATTTTAGAAACATATGTATGATGTTACGTTGGAAATAAGAATCCTCCTGCCAGC
TGCTACCCCAACTTCCAGCAATAGATCACAGGGAGTGGTGTGAACCCTGCTCTTGGGTCAGGCGGCCCAGGCTCAGAACCCTGCTCT
GTCACTTCCTGACCTGGGCTACCTGGGCATGTTAGTTCCTCTCTGTGTCTGTCTCCTCACATGGGTAAGAGTGCCTGCCTGGCTGGT
TTCTGGTGAGGGTGAAGTAGTCCAGGTAGCCCAGTGCCTGGCTGGAGTCACTGCTGAGAAGTGTAGTTCTTTTGGTGCTGCTTTTAT
TCAGGCCTTCAGCAGATCTCACCTGGATCACCCCAGAGCCTGCTAGCTGCTTCCACAGCACGTGACTGCCACCTGAGCGGGCTTCTA
AATTCAGAAATGGCCTGGAGCTTCCTCTGCTCCAAATGCCCTCAGATACAAACCCAGCCTCGCCCAACCAGCCTCTTCATCTCTTCT
CACCGCTTGTCACATAGAGGCCCCACTGGGCCAGATCCTGTCGCTTTGTCTGCACTCTTACTGATAGGACCTTCTGCCCCTCCCCAT
GCACCCCCATAACACCCACTGCTTGTGCCTCTGCTCCATGCACTCTGTTCTTAACCGTCTCCCCTCTAGACTCAGCTCCTGAGGGTG
TCACTGGATGTTTTGCTAGAAATACCCTTGACTGACATAATACCTGGCAACTCACAGGTGATCCTCATGAAAGTATTTGCTCAATAA
ATGAACGAGTCTCATATTTTTGAGGTCCCAGAATAGAGGGTCACCTGCCGCTAGTCCAGACTTCACCTTTTTTCACTTGAGTTTAGG
GGCACCATGATCAGAGCCTGTTTTAGCTCAGGCTGCTGTAACAAAATACTATAGACTGGGTGGCTTAAATAACAAACATTTCTCTCT
CACAGTTCGCCAGGCTGGGAAGTCCAAGATCAAGGTGGTGACAGATTCAGTTCCTGGCGAGGGCCCTCGGTTTGCAGCCGCCTTTTT
GCTGTGTGCTGGCATGGCAGAGTGAGAGAGGGAGGGCCCTGGTCTCTTCCTTTTCTTTTTTTTTTTTTTTTTTTGAGATGGAGTCTC
TCTGTGTCACCCAAGCTGGAGTGCAGTGGCACGATCTCGGCTCACTGCAACCTCCGCCTCCTGGATTCAAGCAGTTCTCCTGCTCAG
CCTTCCGAGTAGCTGGGACTACAAGGCGCACGCCTGGCTGATTTTTGTATTTTTAGTAGAGACGGGGTTTCACTGTGTTGGCCAGGC
TGGTCTCAAACTCCTGACCTCATGATCTGCCCGCCTCAGTCTCCCAAAGTGCTGGGATTACAGGTGTGAGCCACTGTGCCCAGCCTC
TTCCTTTTCTTATAAAGACACTAATCCCATCATGGGGGTCCCACCCTCATGACATCTTCCAAACCTAATTACCTCCCAAAGGCCCCA
CCTCAAAATACTACCACACGGGGGCTTAGGGTATCAGAATGTGAATTTGGGGGAGTCACGAACATTTAGTCCATACCAGAACCTCTA
TTAAATCTTTCAGCTTAAACTTTACACCTAGGCTGGAATGTTTCTAGGTATGTCTCCCGTCTTCTGAATCTAAATGAAAATATCTGG
GAATGGCTGAGAACATGGGTGATTTCTGAAAGGCTGCGCTTTTGCATGTGGCTGTTTCCACGCTGATGGGGCAGTGGCTCTCACCAG
AGTGTGTAGAGATGGTAGTTTCCCTGCCTGTGAGCAGTGCCACCTGATGGAACTTGGTACCGCTGGCCTCCCCAGCAGCTACTTGAT
TGCAGGATTGTATGGAGACCTAAAAGATGACTACCCCTCCTTCTCCTCTTCTCCTCAAAGGCAGGTCATGGCCACTTTTTAATCTGC
AAAAAAAGGATTACTTGAGTTTCACTAATGACCTTTTTTGACAAGAAAAAATGGGAAGGAGCCATGCACCTTTGAAGCAGCTAGTCG
TCTTTTATCCTTGTATCTCTTTGCCAGGCATGAACTAAGCTGCTCTTGCTCCCTTACATCTCCCTGGACTGTTCAGGGCCTGACAGG
CTTTCTTCTCATTCCTAGTCGTCACTCTGGCCTCTGCAGCAGTTCCTTAGATAGATGGATGTTTGGAGTTTTACAGAGTGGCGGAGC
ATCTTTGACAAGTTTTTATAGGGGCAGGTTCCTCTGTGGCCCTGTGGTCTCTATTTGTTCTTACTGTGCATACTGTTTAGAACCACA
TTTCTAACTCTCCAGGATCACCCCTCGCTGAGTAGCTGTATTTTCTTTGAAATGTCATTCCTCGTTACCCTTTCTCACTAGGGAAAT
CCTTCTCTATCTCTTACTCTAGGAAAACTGTTGTCAGTAGGATCTGGTTTATGGAGGGGTTGGTGAATCAAGAGCTAGTCTCATAAA
TCTCAAAGGGGTCCATCTGGCCCAGCTTTGGGTACACATTTCATAGGCCTGTCTCTCTCTCTTTCTTATCCAGCAGTTGGAAGGAAT
GTGCTTGTATATGCAAAAGCAGAAGAGTCCCAGACCTTGGGGTTTATTAATGCTATTTTTGTATTTCTTTTCTGTGTGGGTTGCGAG
AAGAAGCTTGCCAGGTGGCACACAGAGGCTCCTCTGTGCTGGTGATGAAAAAAAAAAACACAGACACTCCTGAAGTTTATTGGTGTG
GAACCCTTCTGGAGTTTTGTTTGCCTTTTCGTTTCTGCATGTGCATAGAAACCTCTTTCAAGAAGAAATAGTTACTCAGAACATATT
CCTTTATTGCAGAAGCATGGGGAAGAAGCTGCTTAAATTCCAGAGACAGCTGAATGAATAAGGCACTAATGAGGAGAGGGAAGGTTA
GCTGGTTTGTCCTTTTTTTTTTTTTTTTTGAGATGGAGGCTCTCTCTGTCTCCCAGGCTGTAGTGCAGTTTCGCAGTCTCAGCTCAC
TGGCGTGGGCAAGGTCCGCCTCCCGGGTTCATGCCATTCTCCTGCCTCAGCCTCCCGAGTAGCTGGGACTACTCCCACCACCACACC
TGGCTAATTTTTTGTATTTTTAGTAGAGACAGGATTTCACCACGTTAGCCAGGATGGTCTCGATCTCCTGACCTCTTGATCCACCCG
CCTCGGCCTCCCAGAGTGCTGGGATTACAGGCATGAGCCACTGCGCCTGGCCGGTTGGCTGGTTTTTCACATTCTTACTGAGACATA
CTGTGTTCATCAAACCACTGAACAGCCAGATCTAACGGGGATAGAACATAGTTCAGCCACTGGAAGGTCACTCCAGAGTGGTGACTG
ACAACCAGTGTGCCAGTCAGCACCCCAGCAACAGAGGTATCCTTGCTCCTGGTTCCATGTGCCACCATCGTGGCAGCACCACTGCTG
TCTTACCGCAGTCACTGACTCCTCCCTGCTTTCTTCTCTGCTGCACCACTTCTCAGTACCCTAAGCTTCCACCACTCATGGCTTCTG
CCTACTCCTGGCTTCAGCTTCCTCATGGCCTCCCTCTTCTGCCTTTTCTCTCCATGTCTTAGATTATTTCCAATTCCCCCCAAAAGA
AGATCTTATTAACAAGTTAGTGACTGTCACCTCTGTTAGGCATGATCATCAACTGTGGCCATGGCTTTCCTTAGAGGGGGTCAGCCA
GGGCAAGTACCCAGAGCAAAACAGTTTCTTCTCCAGTGCAGTTGGGTTAGACACTCCTCCTGGGTGCCTCTACAGTGTCTGGTGTCT
TCTTCTTGTTCTAATAATTATATTATTAATATAATAGTGATAACAGTGATTATGGCAATATACTTTTATTTAATTCTTATTATATTC
GAAGCACTTCACATCGTGTTTGGATTTACTTGGATTGTTTCCATCATCCTCATAATAACCTTAGGACATAGAGTACCATTTTCACAG
ATACAGNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNTAGCATTGGGATTCAAGACAAGCTAGTCTGTATTAGTGTCTGTC
TGTTTGTCTGTCTCCCCCATTGTCCAGGCTACCCCACTTTCTGAGTTTTGGATTTACTTGGATCCCAGGCGATATTGGAGGTGAGCC
TCATTGGCTTAAGTACAGTTCAGGATGGTTTGTTCAGGAGGAGGTTGATTCTGGAATGCTCTCTCTGCCCCACTCTAGAGCAGGCTA
GTCTAAGTGGCTGGAATATCAGAGTCTCCCTTGTACCAAGATGGGTGCAAATGGATACATTGTTGCTTGTTGGCTCTCTAAACTTCT
GTTACAATCCTATTGCCTCAGACATGGACCACTAATGTGTCTTATTTCTTCTTAGACATATACTGGTCTCTTTGCTAAGTTATGAGA
GATGCTGTCTGGTACCTAGTGGATAATGGAAATCTCCTATTGCAGAATCATCTCTTAAAGGCTTCAAGTGGATATGCCAGAGTTAGT
TGTCCAAAAGCTAATTTGCAGAATGGCCAATTTGTTGAATATATTTTTTTCTTTCAACTTTTTAAATTGACTGCTATTCATTTTGTC
TTTATAGCAGCACTTTATGTTATGGTCAATTTGTTTTTTGCCTCTGCTGCCTGCTGCAACAACAGCTGTAGACCTCAGAGGCAGGAG
AGAGAAGATTAAGGATCCTAAGAAGAATATTGACATAGCATATAAGTCTGCATACGAAGAATTCTTACGAATATATTCATTTATATC
AGTTCTTACTAATAAAATTTTAATGTATTAATAAGATATATGTAGAGGAATTCTTCCCACATAGCATTCTGAATCGATCATCTTGAA
TATATTTTTCTCATCACCTGCAGTCTTGGTCTCTGGCTCTCTCTCATTCTGAATCTTTCTGTTTCTGCTACTACTGTTCAGAATCTC
ACTTTCATATGTCAACTAAAACGGATGGAAATAACtGTGTGTGGCCCGTTACTCTGCTTCTCACTCATTTATCCTTGTTCCAGTTAG
GTTCAGCTTCATGGGATATTTTCTGATGTTATCTGCGAAGTCTAGTACCCTTGACATCTTTAGCCCCCAGGGCTTGCTATGCTTGTA
GGATTCAGGTAGGAGAAGGATGAGAACTTCCTATGGAATATAGATGCCAAGCTCTGTGTGTGCACTTCTGGCATAAACAGGTACAGA
AGAGAGTAGTCAAATAACATTTTGTATTTATTTACTTATTTATGTTTTATTACTTTTTGAGATGTGATTTCACTCTGTTGCCCAGGC
TGGAGTGCAGTGATGTGATCATGGCTCACTGCAGCCTCCAACTCGTGGGCTCAAGTGATCTTTCTGCCTCAGCCTCCCAAGTAGCTA
GGACTACAGGCACACACCAGTGTACCTGACTTATGTTTAAATTTTTTCTAGAGATGGGGTCTTGTTTTATTGCCCAGGCTGGTCTCA
ACTTCTGGCTTCAGGCAATCCCCCTACCTCGGCCTCCCAATGTATTGATTTTTTTCCGTGTTTGAGGTTTTAAAAGTTGGTATTTAA
ACTCAAGGGCTGAACATTCAAGTTTACATGCAGAATTGGAGGTTAAACCATGTTATGCATTATTTGTCAGTAACATAGACAAATCAC
CTAAAAGTTCCATAAGAGTTCCACAGAACTGCTCATTTCATTGATGATGCTGAATTCATAGTGAGTTAACATGAATGAGACTGTTTA
CACCGGCTTAATTACTACGTTGACTTGAAATATCAGCCAGCTGGGGAAAATGACCATTTAAAACCTGGCACATCCCTTTGCTCCCTA
TACCCACATTTTTTCCCAGCTTCGACCACGTGCGTGTGTCTGTGTGTCTATGTGCATGTCTGTGTGTGCGTTTACGTGCATGGAGGA
CTGAAGCATAGTGTATTCAAAGCTTTTGTGTTGTGCAGGACTTCGCAGATGCAAATGAAGACCCAAAGAGAACCAGCATAAGCAATC
AAGGGAGTGTATCTAAGGCTCCTGGAGTGACACATAGAGCTCTAGGGCAGGCCTCAGGAACCACTGGACTGGGGATTTATAATGCTG
TCTGGATTTTCCCTCCCTACTCTTGTTCTTTGTTTCTTCTTGAGCGCGGTTTTCTGCTTCTCACTGCTGTCTGACTTTCTCCACTTG
GTAGGAAATATGACATGCTTCACAAGGCTCTGGAGTTTGGCATTTTAGAGTCTCAGCCACTAGAGAAAGGTGCCTGTTCTAAATCCT
GGCACTTTAGGAAGGGAATCCAGGCTTCTCAGCCCTCACCAGGCCAGCAGGATTGTATGAGATCTTGGCAGCTCTGAGGGGTCATGG
AGTGAAAGGAGGGGCGAAGGCATCCTTACAAGGAGGAGGTGGTGCTGTTCCCAGAAGAGGCGGGGGCCTCAGGTTCCCAGAAGACTA
GGTGTCTGCTGGTGGTGTTCTTGCACATTCCTAAGGATTTACTGTGAATCTGGTGCCTGTTTTAAATTGCTTGGCAGACTTAACTGT
AATCTTAGATTTTTTTTCTTACAGCTTCTTGTCAACTACAAGGTTTAATTATAATTACTCTTCAGCAGGCATATACTAGCACTTCTT
ACTCATCTGTCAAATTGAGCTGAATGAAATTAAAAAGTCCAGAAAGAGACCCCATCTTTAGGACAACAGAAGACTGAACAGTCAACT
CCCTATCATTTTTGTAATCCAAATCACTGTTGTCAGGATTATGTTCCCCCCCACCTTTTTTTTTTTTTTAGACAGAGTCTTGCTCTG
TCACCAGGCTGGAGTGCAGTGGCTCAATCTCAGCTCACTGCAACCTCTGCCTCCTGAGTTCAAGCGATTCTCCTGCCTCAGCCTCTC
GAGTAGCTGGGATTACAGGCAGGTACCACCACGTGCAGCTCATTTGTGTATTTTTAGTTGAGACTGGGTTTCACTGTGTTGGCCAGG
ATGGTCTCGATTTCTTGACCTCATGATCTGTCCGCCTTGGCCTCCCAATCCCTCTGTGTAGTCAAAAGCAATAAAACTGCTGCTCTT
CAGTGAATGTTGGGAAATTGTGCACAGATGAAAGAATAAGTCTGTTTTTTTTCTCCTGCTAGGTAAGTAATCCCTAATTTTCCCTAA
TCCCAAGGGGAAAAATACTGGCCATTGGGCATTGACTTCCACATTCACTTCTTGTGTTTCAAGATTTGAGGTAGTGATGATGGCTAA
TAATATATTCTTGAAAAATGCCGAGTGGATGTAAAGTGTTCTCACCACAAAAATGATAACTATGTGAGGTAATGCACATGCTAACTA
GCCAGATTTAGTCATTCCACTATTTATATATACACGCACCTCAAAACATGGACATGTACAATACATACAATTTTTTTTTTTTCTGAG
GCAGAGTCTCGNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNCCAGGCTGGAGTGCAGTGGTGCGATATCGGCTCACTGCAAGCT
CCGCCTCCCGGGTTCACGCCATTCTTCTGCCTCAGCCTCCCGAGTTGCTGGAACTACAGGCGCGTGCTCCCACGCCCGGCTAATTTT
TTCCATTTTTAGTAGAGACGGGGGGTTTCACCATGTTAGCCAGGATGGTCTCGATTTCCTCACCTCGTGATCTGCCCGCCTCGGCCT
CCCAAAGTGCTGGGATTACAGGCGTGAGCCATCAAGCCGGGCCAATACATACAATTTTATGTCCATTTCAGTTTTTTAAAATTTTAA
AATAATTTTAAATAAGTAGATAAAGGATATATTCTTTTCACCCAGGAAAAAAAAGATTTGAGTTAGTTTTCAACTGATAACTACGTG
GGTATATTCTGAATCCATTAAAAATAAAGTTCTTGGTAGTCTTTCCTATGCTTTGTGGGAGATTAGTTCCCCCCAGCACGCTGTAGT
ATCTGTTTGCAGCTCTATCTTTGCCAGAATTTAGGATTAAATAAGTAGAATCTGCAGTCCAGCTCAGCCCCCCAATGATTGTTGTTT
CCGTGGAAACAGTTGAAGGCTCTCAATTAAAACTGCCTCTGAAGGCTGGAGAGAGTAAGTATTAAAATGAGTTTGTCTGAGAAGCCA
ATTTTTATCCTCCAAATTAAACCCAGAGAGTGCAGATGATCTAGGAATCTCCTGACAGTTGGGATCATACTTCTTTGTCTTTGGGGG
AGGGATTATTTTGCTATTTCGGGCATAGGTATGTTTGCTAGCTTGTTGCTTGCTTTGCTGAGTTGCTCAGATCATCGTCTTTGTTAC
CCACTGAGCTCTTTATGTACAACCATGGATCATGGGCAAGGGTTTGGGAGCAATCTGATCTTTGTTCAGACTTGTGAGCCGCAACTA
AAAAGCCCATTAAAAATGGATGAAATCATAGAACGGTCTCAAAGTTGAAAGGAATCCTAAAGGTCATTAAGGCCTAATTTCCCATCC
ACTTTGGGAATCTTTCCTAGAATGCTGTTGACAAAGGTTCACAGCATCAGCAACCCTGTTGGGGAATCCCGTATTTCTCAGGGCAGC
CTTCTTTGGTTCATTGAACAGACTTGGTGGTATTGTTACCGGGGGTCCTTGTTCTTAGAGCTCCCAAGATGGTGGCGGGCCGCTTCC
AAGGTGGCGGCAAGCCTCTTGTTCTCTGACCTGGGGTTCTTGGCCTCACGGATTCCAAGGAATGGAATCTTGGGCCATGCATGCGGT
GAGTGTTATAGCTCTATCCAGCTCGATTAGGATGAACCCTGGGCACTTAGACCGTGCAGGAACAATGGCGAGCCTCTAGCCCGATTA
GGAGTGGCAGTGGGCACCTCGCTGGATCAGGAGCACAGCGGATACCCTGCCGGATCCGGAGGGGTGGAAGTCAGTGGTGGCTTCTAC
AACGACGGCAATCAGCAGTGGTGGACGGCGAGTGAAAGCTCAGTTCGAACCAGAACAAACACAGACCAGAGAGTGTGCAGTTTGCAA
GATTTAATAGAGTGAAAACAGAGCTCCCATACAACGGGAGGGGACCCAAAGGGGTTGCCCACTCCCTGCTTGAATGCCTGAGTTTAT
ATCTCGATCATTGTCCCTCCCCCTGTGCTCTCAGGCGATATATGATTTGACAATTTCTTTACCTCCTGCTTTAGCCTAATTTGTATT
TTAGTGAGCCTATTTACTACCTGATTGGTTGGGTGTGAGCTGAGTAGTTACAAGCCCCATGTTTAAAAGTGGGTGCGGTCACCTTTC
CCAGCTAGGCTTAGGAATTCTTATTCGGCCTGGGAAATCCAGCTAGTCCTGTCTCTCAGTATTAGAAATGCCTCTTTATGATGGAAC
TGACATTTCTCTCTGTCAATTCTGCTACTGCTACCTCTTCTTTCTGGTGCCAAGTTAGCTGTGTCTTCTTCTCCCATAATTAAGCTT
AAAGTCTTTTAAGAATGTCTTCATGTATCCCCAAAGTCTTCTCTCTCTTCCGGTGGCTGAGAAGGTCCATAACCTGAAAGCTTCAGG
TACTAACATCTGTGGTTAAAGGCATTGTCAGAGGAGAGGCTGGTTTCCTAACAACCCTGGGCCTTTAGCACTGTTGCTTGGATTTTT
TATTTTAAGTTTATTTTTGTAGAGAAAGGGTCTCGCTATACTGCCCAGGCTGGTCTCAAACTCCTGGCCTCAAGCGATCCTCCCACT
TCAGCCTCCCAAAGTGTTGGGATTACAGGTGTGAGCCACTCCACCCAGCCTGTTGCTTGGATTTTGTCCGACTTCTCTGGCAAGTTT
TTTCTCAGCATCATTTGTATAGGTGGAGAATTTGGTTTGAGTATTTCTGTTTTCCTTCAACCTTTTCCCCCGGAGTAAATATTCATA
GTTCACTGCTTGGAAACCCAAGAAACAAAATTGGATCAAACCTTTGGTCTATGCAGACTTGTGAAGGGTGTGGTTGCCTTCTTTGAT
GTCACCTCAGAAGTTAATGAATACAACCTCAATCCTAACTGCTTTAATAGCCTTTCAACCATTTGTCTGTGACATTAAACCTTAGAC
TTTTTAAAATTAGGTTTAAGTTTTACATTATAAGAGTTCTTTATTTCCCCATATACATTCTGTTCTTAATATCCTATTATTGTTCGT
AAGTTGACACCCCCCTAAAGGAAGAAACAGACAGAAAAATAGAACAAACGAAATACGCTTCTCCAAATGTTCCTACCTACAACCTTT
AAATGGATTAAAACTTAAAACTCCTAAGTTCAGATGTTGGTTAGTGCTGACTGGGAAGTACTGGGAAGCAAGTGTGTGGCCCCTGCT
TGGTACTGAAATAGACATTGTGTACTGGTTTCAAAGAAGCGATGGCGTCTGTCACCTTCAGCCCATCACCAAACTGCTCTGAGTTCC
ATTTTCCTCAAGTATAAAATCAGATGATTGCTTCCTGTTCTTACAGGTTATTGGGAGTGCTTTGAAAACAATTTTTTTTTATTATTA
AGGCTTTACAGCAAACTATACTGTGATTCTCTGGAGGTATTTTGATTTGAGAGTACCAAACAACTTCAATAAAATTAAAATATTGGT
GATATTGGCTGTTTAAGGCCCATGCACATCCACAGTTTCAAAATAATCTTGTAACATAGGTTCTGGGTGGTATTTAATATGGTATCA
CACATAAGGGAAGAGGGGATTTCAGTAAAGAAATGTCATTTTTTGTGGAAAAGTGAAATTACTCGAGGTCCGTTTGGTGTTCTTGTG
GAAGAAGAATAAGAGTTGAAAGTGAGGAAGTAACCTTTGATTTGTAAATCATTTAAATTTCAACTCGAAGGGGGTAAGAAATACCCC
AACAGCAAGCCACAGAACAGCCACCAGAGCAAACCACACAAGGCTTTTGGGATCTCAGCCTAATAAAGGTCTCCCATGGAGCTCAAG
AGTAGATCCTGAGGTACGATATTTTCTTGGAAAAAAAAAACTTGTAGGTGTTTGTAGCCGGACCGGGTGCTGTGTTAGTTTTCAGCT
TCTAGCTCGATCCTATTTCCTGAGAAATGCCAAGGGCTTTGCAATAGGCATTTGTCTAACTTCTGCGTGAATTGGGTCTTACAGAAG
ATGACCTCCCCCCTCTCTGGGGGTCAGGGGGTGTGTGTGTTTTTTATACATCTGTCTTTTTTTGTTTGTTTGTTTTTACTGAAGACC
TCTTATATTTGCAGACACTGAAACCAAAGGTAACACCACGTCTTCTCCTTACAGGGTCTAATGACAGTTTCTTGGGCTCTTCTTTTC
TTTTTTTTTTTTTTTTCTCTACATATAGTTTTCCCAAAAATATGAACACGTTTAGATGTTGGAAAGAAAGCAGTTTCCCTCCCTCCT
TCCCTCCATCCCCCCCTCCCTCCCTTCCTCGCTTCCTCCCTTCCTCCCTTTTGTTTCTCTCTTTTCTCTTTCTCTCTCTTTCTCTTT
CTTTCTCACTCTTTCTTTCTCTTTCTCTTTCTCTCTCTCTTTCTTTCCTCTCTTAAGTATTAATCACATACCTGATAATCAGATGCC
CTCTGAAGGTTTTCTTTGGGAGGGGAAAGGACAAATGCAATAATCTTTACATGTTGGAAAACTGACCCCACATCCCCGTGCCTTGTT
GTGTGTTCTTTTCTGCTCCCAGGGAGTTCTGTCCAACATCTCTTCCATCACCGATCTCGGCGGCTTTGACCCAGTTTGGCTCTTCCT
TGTGGTGGGAGGAGTGATGTTCATTTTGGGATTTGCAGGGTGCATTGGAGCGCTACGGGAAAACACTTTCCTTCTCAAGTTTGTAAG
TATTCCACTCTCTACTCTCAGATTGAGAGCCAAAACTTTACCTTCCATCAGGAGGTCACCCTATCTACAGGCAAGACAGTGAGCTGT
ACCAAAGGCACTGGCCAAAGTAGGGCCCAGGTCATTATTTTCTTTTGAGAGAGCTCTCTAGCCCTGATTCCCAGTTGTGCCTCCTTA
ATAAGTACAACACCACCCCCACCACCCCACCATGCCATCCATGGAGGTTTTGTGCATATATATCACAGACTTGGATTCCATTCCCTA
AGATATCTTTAGGACTAAGGTAGGTATATATATATATTTTCTTCTAAGTGATTCCTTCCCACCGTTTTAATGCACATAGTAAGTGGG
GAGCTGTGCAGGCTGTTGGTTTGGAGAAACCAGGCAAAAGCACAGTGACTGTGGCCTGTCCAGATTAAATCTGTTAAGCAGTAGGTT
TTGCTAAATATGGAGAAACAGTAAAAGGTAAAGCTTCTTTGTCCTTAGCTGGTACAAAGTTCTCCTTTGTAATTTTTAGATGTGGTG
CGGTTGGCGTGATCACTGTCTGAGTGAATGTTTGTAGACGAGAGGTTTCACCAGGGGGTTCACTGGTATTTAAAGTTAAAATAGAGA
ATGAAAAGTGAAAGCCTTTGCGTTGGAGACTCGTTTCAAACGAGAAGAAAATGGCATGTGCCGTTTATGTATCTTATATAGGGTTGT
TGAATGTAGCCAAATGTGCCTGTCAGCATGGAGCTTGGCACTCTGATGGGCGTCTGAATCATAGCTCTCTATACGAAGCATCCATTC
CCCTTTCTAAGAACAGAGAAAGGATCCAAAGCACCCACCTTCATTTGCTGAGGAAGGATGGGGATGGTGGAGATTTCTAGAGAACAG
TTGTAGTGTAATATTTCCTGTTACCACAGTTATTCCCAACTTTTGTTGACACATCATCATCCAGGGACATTCTCATTAAAAAAAAAA
AAAAAAAAAAAAAGCATATCCAAGAAGTAGCCCAATAGTCATGTAATGTGTGATTCTCTTCCCCAGCATTTCTGCCACATCATCCTC
CAGCGTCTTTTCTTAAACATCCCTGAGAAGAGGGTACACGGTGTGCCCCACCCAGGGCAGCCCATTTCTGAGCAGCTCTGTCTCGGA
GAAGTTTTTGGAGGCTATTTAACCTGAAAATTAGCATGACTTATTCTAACAAAGAGAACCGAGGGGTACCTGGTCTTACAGATAAGT
TCACAAGCAGCTGGTTCTCTCTTGTGGATACATGGGACCACAGTGGCTGTAGCTAGTTTCCTAAGACTCCATCTGCCTTATCTGCCA
GATCCTTCCTTTGCCACACACACACACACAGTCTCTCTCTCTCTCTCCCTCCCTCTCTCATACACACACAACCAAATGCTTCATTCT
TCACCTAGTCAGAAACCTGGGGAGCCCCCAGGAGAGCTCTGTCCCTGATTGCCACCTGCCAACCTGCCAGTCTTCTTGGCTTGAGGC
ATCTCAGTGCTCCAGTGCTCAGCTCACCTTGCCAGCTTTCCTGTAGCCTGATTGGGTCCTGGTCATACTCATCTGTTGCCACTATTT
CATCAGTTTCGGCCTCCATGCCCCAAAATGTGGGATCCTTTTTTTTTTTTTAGATGGAGTTTCACTCTTGTTGCCCAGGCTGGAGTG
CAATGGTGTGATCTCGGCTCACTGCAACCTCTGCCTCCTGGATTCAAGCGATTCTCCTGCCTCAGCCTCCCGGGTAGCTGGGATTAC
AGGGGCCTACCACCGTGCCTGGCTAATTTTTTCATTTTTAGTAGAGATGGGGTTTCACCATGTTGGCCAGGCTGGTCTCAAACTCCC
GACCTCAGGTGATCCACCCGCCTCAGCCTCCCAAAGTGCTGGGATTACAGGCGTGAGCCACCATACCTGGCTGGGATCCTCTGAATT
GTATTTTTTTTTTTTAAGTAAAGATGGGGTTTCACCATGTTGGCCAGGCTGGTCTTGAACTCCCGACCTCAAGTGATCCACCTTGCT
CAGCCTCCCAGAATGCTGGGATTACAGGCATGAGCCACTGCACCTGCTCAGTCCCCTTAATTGTGATTTACTTTAATATAGTTTCAT
TTATAGTGGTGGCCCCTTTAATATAGTTTATCATTTCTTACAGTTGAAGAGTCCAGTGTTAACTGAACAGGCAGAAAACTACACATT
TCTCTAAATCAGCACCTTAATTTATAAAGTCTCATGTTGGAAATGAAGAGAATTGTGTTGTTTCTTGGCTTTTAAGACTTGGAAAAT
ATCTTAAATGGCCTCTCTCCATTTAGGACAAACAATAAAAACTTGCCTGGAATAATCTTCGAACAGAAGGTATGAGATAAGTACTAA
GTAAACGTCCACAGGACTCCAAATAGCTCTAAAGATCTGATTCCCCTAGGAGACTTGCTGAGGGAAAATCTAGGAACTAAGATACTG
CAAAATGGAAATAGACCTTCTTGGTTGACCGAGGCTCTAGGGTGAAGCCTGTGAGTCAGTGTCTGCACCTTTGGAAATAGAGGAAAT
TAGCTCAACAGGGCTTGGGGCATGTAGGTATCTGCCAAATATTTTATATGAATTGAATTTGGACTCAATTTTTGGTATGTATAATAT
GTAACATGAAGAGAAAATTTACCAGGGGAACTGGAAAATGCACTTAACTTTTATGGAGGTGAGAACAAAAACTGAATTAAAATCTTT
CCCTGACTTGGTGTTTCCTGTGTATGCTCATGCATGCTAGCTATAATTTTGTCATTTCTGTACTGTGATAATTCATAATTCATATTT
ACATGCTTAAGGTGGGGCCTAATACAGGGCAGGGCGTTATTTACACTGTAGGGTAGGCATTGAGCAGAAGTGGTGTGATATTTTTTC
TGTTCATTTTTCTAGATTGGCGAAAGTTTAAATTGAAATTATTGGCATCACTGATGATAATTAAGAACTGTTTAAATTCCATTTCTG
GCGAATTCCTGAAGGATATATAATCAGGAACTCTAGGGAAACCTGAAGGGCTGAGCTGGAGGGTCTAGAATTGGGGCCTTAACCTGG
GAGTCCAAAAACTTTGCCAAACACTTATTTCCACCTTCCACTCCTCCTCCCGTGCCCACCCTCACAGAGCAGTACTTGGCACCCTAG
ACAAATACTGCCAGATCTACAGACATCTAATGTCTGCAGATACCCACTCCCTGCCTAGCCTCCCTAATCACATAAGACTGGGACATC
TTCGAAGGCAAGGATACCCCTTGTTCTTACTCATCCTTGTTTCTGCAAAGCCCACCTACCTCAGGGCCTGACTCATTGTGGGTCATT
CAGTAGATGTTTGCTGAATGAATAAACATTGTTTGTCTTTTGTTCTTTTAATGAGTAACTGCTGTTTCCTTTGCACCCCTCCCCAAT
ACATTTTTCTCTTGGCTGGCAGCTCCTTGTCTGCTTGCTGAAGATAGAACCGCAGACCACCTGAGCACCCCCTGCCTCCTCAGCTGC
TCACTGTTCTTCTGCATGTGGCCTTCAGGACCTCTGCCCAGCAGTTGTGGCCCATGTCACCCTCTCTGCTGTTGTCACCAGAAATTT
ACCCGCACTGTACCCTCTGGTCCTTCACAGCTCATTTCTCATAATCTATTTTAACAACAAAGTTTGAGAAGGTTAAGAATACAGGAT
GGTAGGAGGTGGAAAAAACAGTGGGCGTGAGCAGTAAGTCGATATGACTAAGCTGAGACTCTTCTGAGTCTTCTAGATGGAAGGGGA
ATTTCTCCAGAGTTACGGGCAGTTACCACGTTGCCTCATGGTCTTGAGCCCCTCTTTTAAAACCCCTGTTGTCTACATTAAGCGGAA
GTCTCCCCTTGGCTGCATTTTGATGTCACTTAAAATGTTGCTGGGGCCCATGCTTCCTAGAGGAAGGCAAGCACAGCTAGCATTCCC
AGGAAAGCCCTCAACTCAGGGGTTTCATTTTGTTTTTATGATGGGAAATAACTTTCATTTCGCGGTCACTGCCCACCCCACTACTTC
CTCTTTGACCCCTTCATCTAAGATGAACACATCTGTTATTTTACCCTGTATAGCAGGTGCAAAAGAGAATTAAAATGGTGTCTGAAT
CCATAAAAGGAATATATTGCATAGAGAATCAAGGCCACAGTAACAAGTGACAGGCGATCTTAAAATATTGGAATAATCTATCTTTCA
AGTTACAGAAGCTTACTAGAGATAGGGGCAGGTCATTGTTACAAGCATCTGGCAGTACATTAATCATGAGAAATTAATTAGGCCTTA
ATGTGGGTCTAATGAGAAATCCTTATTATCCATGTTTGCTTATGGGCTTTGTTGGTTAATTATCAAATGGAACACTAGAGACCCTAA
AATGTGATTATAAAATGATTTTTAAAAGTCCTTTCTTGGAGTGTAATATTCTATTAATGAATCAGCAGAAACTGAAGATTATCTGCT
TTGGAGATGTGTAACAATCCAATGTAATTACAAACCTGTTTAGAGAGGAACCATTTAGCTATATATGATCAAAATAGCCCTTATAAC
TGTTCTCTGTGTATGTGTCTTTTATCTTAGTTTTCTGTGTTCCTGGGAATTATTTTCTTCCTGGAGCTCACTGCCGGAGTTCTAGCA
TTTGTTTTCAAAGACTGGATCAAAGACCAGCTGTATTTCTTTATAAACAACAACATCAGAGCATATCGGGATGACATTGATTTGCAA
AACCTCATAGACTTCACCCAGGAATATGTAAGTTTAAAGTGGTTTGTTTTCAAGTTGAAAGTTCTCTGAACGATGACCCCAGTGAAT
TTCATCCCTCCTGGTTTCTGCATCTGCCAGCTGGAAACTACCTGACTATTCTGCATGGGTGTTTGTTTGTTTGTTTATTATTTTCCG
ACTTGCTCTGTGACTTACAGGATTTGTATTCTAAATGGAAATTTCCTATTTTCACTTGTTTCAACTTCTGTTAAGTTAGAGGAACAA
AGAAGAAAATGCTTGAGCAAAACGTTATAAACATGGCTGTCCTTTCCTATTTGCATTTGAGGAACATTGTTTTGCTCCGGGTTTAAA
GTTTTCTTTGGTTATTAAATATGGATTCTTTCTTATCCCTTGCCCCCTTTTTTGGTAATCCTTGATATTTCACAGCTCATAATAATC
TACTTATCAACAGAATAATTTGCCACAAAGTTCCTAAAATGTGCCTATCCAAATATTATTATTGCACATGCAGTTAAAAGCAAAAGC
TTAGAAACTCCTAAGTCCTAGTGGCTTCTAACAAGTGAAGAATCTGTGCAGTGGAATCCGGGCCACTCTCTCCTATCCCTGAGCATT
GGAGTCCAGGCCACTCCCTCCCGTCTCTGAGCATGCCTGTGAAATGGCTCTTTCAGTGCCAGCAAAGCAGCAGATGTCCCGTTTCAC
TCAGAATGCAAGGGACCAAAAGACACAGGTCTAGTGCCAAAATCTGATAAGGGACCCTGGGCTCACAGGACCTTGACCTATTTCCCC
AATAGCAAAATGGAAATAATACTAGCTACTGTCTAACTCACAGGGATATTATGACACTAATGAGACATACATGTTTTTATATTAGGC
TAAGGTGTTAGGGTCCTACAAACTTGCAGGTCTTTATATACCTGCACTGAAAAGTCGGGTAGGCTGAGCTTGGACTTGGACAGCGGT
GTCTGTGGCATTCATGTATATGGCAGGACCGGAGGGCAAGCAGAAGCCCAGTGAACGGTAGCCTTCTTGGGGTTGGATAGTTGTCTG
CCTGTGATGAAGGGAGGCAGCAGCCATGTTGACACTCTTTATGAGGGAGGCCATCCCCTTCCTTAGGCACTCAGGACTGAGCAGGGT
TATGTGGTTTTGTTCAGTGGCAGCAACATTTCGTCCTGGAGTAAGCTGCCCCTTAAGTGGGAAATTAAGCCCTGATTCATGTCCTCA
GCCGACTTCACTGTTTTATTAGGTCCTAATGGGTGCAGGCTCCCTAATAATCCCTTTCCTGGCATTATTCCTGACTAGTTTGCATGA
CACATTGATGGAGCTTTGTGGACACACCTCGAAATGCATGACCCAAGCCCTGGAAGGACCATGTTGTCTTGGGATGCTGTGTGCTTT
AAACGGAAATACTACAGTTTCATGTGATTTTCTTTTTGCATCATGGAATGTAATGTTTAGAAGTAGCTGTTGGTCCGGAAGGTCTTC
AAATGGTTGAGACTTGATATTTATTTAACTCAGAGAAGTAGAGAAAGTGCATCCCAGGGAAGTGAGGAAGGGGCTTGTGTAGTAGCC
TTTGACTCCGGCCCCATTTAAGAACCACTGAATTTAACCGCTTGTTTTAATATATTTTAATTGGAAGACTGACTTTTTAAAGTTGAA
AATGTAAAACACATGCACTTGTTTTTAAACAAACTCAGAGTAGAAACAAATATGATGAAAAGTTAACCTCTAAAATTTTTGGTGGTA
ATGGATATATCAACTATATATCCATTGATTGTGGTGACATTTTTGTGATGTATACAGATGTCAAAATTTATCAAATTGTATACTTTA
AAATGTGCAATTTATTGTATGTCAGTTATACCTCCATTATTGCGGGATCTGGCCAGCAGCCTGCAATGCAACAGGGATCTCTCTGTT
CCCAGGCGGATCGGCAGGTCGAGAAATAATAGACACACACAAGATAGTGAAAGCTGGGTCCAGGGGGGTCACCGCCTTCTGGTCACG
CAGTGCCAACAATGCACTGGATATACCAGAATTTATTATTAAGTTTAGTGAGGGTGGGGGTAGGTTAGTGAGGGATTTAGGTCATTT
GATTATGAGGTGAGATGGTCACATGGGGATGAAGTAATTCTTTAACATAACATCTGTATGCAAAAGTACAGTATACAGGGATAAGAA
TTTACAATATAGTGTGTGCTTCAGTAATTTCTAACAGAGCCTTAAAACAGAAACACAGTCTTTCCACAACCTATGATTAGCAAGATA
TTAGTCAGCAGTAACAGTTGCAGCAAAAGCCAGTTACAAACAATCCATAGAAACAGGACGTGAAGCTACACAACCAGTTAGACCAGA
AATTCTCAGAACGGAGTATGCCTTAACCCTAAAGAGGTCTAGAAGAGCCGTGGCAAGATGAGGGCATTTATAGCCCTGTCTTATACA
TATGGACAGGTGCCCCTCATGTGTCTGTTTATAGGCTCTCCACAAAGGTCGCATTCTATTCCCAGAGCTATGAACATCTGCTTTTCT
GGGATAGGAATCTTGGTGATGTGAAACCTCCCTGACTGCACGTCCATTCATAGGCTCTCTGCAGGGGGAAGCACATCACGCACTGTT
GGCTCATTCTGGCAGTCCAACCTGGCATTGTCTTTACACAATCCTGCATGCAATTTTGTATTTCCAGTAATCAGGAGCATTTCATCT
TTTATTCCATAGCAATAGTTTCAGGGGGTCTCCCTGCGCTCCATAAAACAAAACAGATCTTTAAAAAAACTATTAGAATGTAGAGAT
CAAAACTTTGAAAAAGTAAGCTATAGTTAGCCCACCCATAACCCCAGTTGCCTCCTTCAGTCACTACCACTGTCGTTAGTGTCTTAT
TTACCCTTCTAGAATGTTCTTGTTTCTCCTTCTAGAATGACGTGCCCAAGCCAAACAGATCTTGTTCTAACTGAGCCCTGCTGTACA
GCTGCTCTGCCCTTCTTCCACCTAACACCACAGGTCTCATCAGTTGTTTAAATCACGTGCCAACACATCCCTACAAACCTTCAGCCC
TAATTAATTTGGAAGATTTTATATGGGCCATTTAGGTTCAGTTTGGCTTAAAAATAAAGTCTAGGACAGAAAAGAAGGTCAGAGCTG
GCAGAAGCTGGTGTCGTTCGGCGGGTGTGAGTGTAGGTGACTGCTTGCCTCCAAGTTGGGATGTTCTAATTCTTTCCTGTTTGCTCT
AGTGGCAGTGCTGTGGGGCTTTTGGAGCTGATGATTGGAACCTAAATATTTACTTCAATTGCACAGATTCCAATGCAAGTCGAGAGC
GATGTGGCGTTCCATTCTCCTGCTGCACTAAAGATCCCGCAGTAAGTGAATGCCAGCGAACTATTGGCCTACTCTGTACATCATGCC
GAGAGTGTGCATGGGACGAGCAGAGTGATTTTTTGGTTGAACCCTCTTCTTACCTGGTTACTCGTATTTGTAGCTCCTGTAGGGGGT
TATGGTGCTCAAATCTTCCTAGTAGGAGTAATAATTGCTGGCATTAATTTAGCAGTGTGTGCTGGTTCCTATTCTAAGCCTTTATGT
GTGTTGGCTCATTTAAGTTGGGCACATTCCTACTGGCAGATACCACCATTACCCTCCCTTTTCAGGGGAAGAAATCAAGGCACATGA
GGTGTAAGTGACTTTCCCAAGCTTGCACTGCTAGCAGGCAATGGAGTGGAATATGAGACCCAGGCAACTTGCCTGTATCATCTGTTA
CCCTTTCTTCATGAGAAGGGCAGGCTCATATGAGAGCTCTCTGCATTGGTTTGGGGTTAGTAACCCTTTTCTTTTTGGCTTAGTTCT
AGGGGAACTATTAGGAGTAGCAGGAGAGTTTGAGACAAGATAGGCCTGGACTGCTGTGTAGGAAGACCACAGCTGCCCAGACTCCTG
AATGTCATCATGACTATAGTCATGCAGTTTGCATTTTATTATAAAAAAGAATAAAATATAAAATCTATTTGTAAGTCACTCACTTTT
GCCACAGGTTTGATTATATTTTGTGTCAAATCGTGCCTGGTATATAGAATGACTTAAAATGCAATTCTTAAGCCAGGCATAGTAGTT
CACACCTGTAATCCCAGCACTTTGGCAGGCTGAGGTGGGAGGATCCCTTGAGCCCAGGAGTTAAAGACCAGCCTGAGCAACATAGTG
AGACCCCATCTCTACAAAAAAATTAAAAAATTAGCCATGTGTGGTGGCACACACCCGTGGTCCCATCTACTCTGGAGGCTGAAACAG
GATCACTTGAGCCCAGGAGTTTGAGGCTGCAGTGAGCCATGATCATGCCACTGCCCTCCATCCTGGTGACAGAGTGAGACCACAATT
CTTTTTTTTTTTTTTTTTTGGTAACTCTCTAGTGTTAGAATGACAAACATGAGAGGTTACTCCACGCATCTCTGTGTGCTCCTAGCT
GCAGAACCCTTCTGGGACTCCTCAACTGTTCTCTGGCCCTGCAGTCTCCAGATGTACTCCCCAAGGCTCGCCCTTACCCCTCAGAAG
CAGAGCCGAGTGGGTGCTGATGAGCTCAGTGGGGATGCTCCTTCTTATGGTGGAGGCTGTGGGGCTCTTAAAAGGAGCTATGGTCTT
GGAGTGGAAAAGCAGAACTTCCCTGTGGGCTTCCTCCAGGGGACCACCTCAGCTGTTTCTCTTGGTCTGAAAGAGGAGACAAAGTCA
CTAAGGACTGTGCTGCCCCGCCAGCTGCCCTTCCCACCAGCTCAAACCTCCTCTCCCTTCTGAGGAGAGCCACATGGCAGCAGAGTG
CCCACTCAGAGCTTGAGGGGCTCATCCTGGTGGCCTTGCACTGGCCAGCGGGTGCCGCACAGTGCGCCTCCTGTCGCCAGTTCCTGT
CCCACATTAACACTCCCTTGGGAGAATTCTGTATTCTCAGAGGCATTAATGCTTCCCCTTTCACCTACAGATCCTCTGGGCAGAGCT
AGCTCATTCCTTGCGCTCTAAGCTAATAAAATCTGCCCCACACCCCGCAGAGAAGGAAAATAAACAACCCGAAAAAGTGGAACCATG
TTTCTGATCAGACCCTGTGAGACGTCAGGTTGTCTGTGTGATTATGTGGTCGGGGCATTGTTTTGTTTTTTAATTATTAAAAGCTCA
TTGAAAAACAATTTAAGTAACATGGAAGTACATAGATTAAAAAGTAGCAGTCCATCCAAAAGAGCATACATGAAGATTCACTGTGCC
CTTTATGTTTTTTATCTGTGTCTCCAGTTCCCTAAATCCCATAAGCTTCTTGAGGTTGGCACCACTTTGAATCTTGAGCCTCCTGTG
TGATAGACTTGCTGTCTTTTCTGATATTCCCCAAGCCTTAGTTTTCCTTATTTGTAAAATAGGGCATATTTTAATACCTACTTCTGA
AGATTGTCGCAATAATGACATGGGATCATGTATATAAAGTGTTTAACATAGTGCTGCAGTCTTTGTGCCCAGATACACATTTTATTA
CTAATGCGTAACAGAAGTCTTCTGCTCATTTCTAGGTGGGCTTTCTGTCTAATGAGTAAATTTCACTTTTCTCTTTCCTTTCTTCAC
CAGGAAGATGTCATCAACACTCAGTGTGGCTATGATGCCAGGCAAAAACCAGTAAGTGGAATCTCATGTTGTTGCTTAGCACGAAGG
GTGTTGGAGGAGCTTTGGCCTGTGCCTGGTGCTCTATCTGTGACAATGTGCTCTCCTCTTCTCTTGTGAAGGAAGTTGACCAGCAGA
TTGTAATCTACACGAAAGGCTGTGTGCCCCAGTTTGAGAAGTGGTTGCAGGACAATTTAACCATCGTTGCTGGTATTTTCATAGGCA
TTGCATTGCTGCAGGTAAGATAAGGGTCCTTTATGGATATCACAGGGAAATAATGCCCTGGGAGAGCCCTTGCCAGGATCGGAGCTT
TGCTTAAAAACCTTGATTGTCATAGTTTTTAAGACACATTGGAGGGCTGAGGAACAGACCTAAACGATTTGGTTTGTGTTTTATTTT
ATTCATTTATTTATTTATTTTATTTATTAATTTTTTTTTGAGATGGAGTCTTGCTCTGTCGCCCAGGCTAGAGTGCAGTGGCATGAT
CTTGGCTCACTGAAAGCTCCGCCTCTTGGGTTCATGCCATTCTCCTGCCTCAGCCTCCCGAGTAGCTGGGACTACAGGTGCCCGCCA
CTACGCCCGGCTAATTTTTTTGTATTTTTAATAGAGACGGGGTTTCACCGTGTTAGCTAGGATGGTCTCGATCTCCTGACCTCGTGA
TCCGCCCATCTCGGCCTCCCAAAGTGCTGGGATTACAGGAGTGATCCACCGTGCCTGGCCTGATTTGTGTTTTATTAAAGCTCTGAG
TTTAGATGCTACTATCATGAAATACTTTAGGAAACCTTGTCTTTTTCTAAGATGGAGACATTAATTAGATAGTGGTTGCCTACTGGC
ATTCCATCGTTTACATGGGCTCAAAAATACTGCATTGTGGCAACTGAGTCAAACATGCATAGTTAGAAGTAGATCTCAGTGGTGACG
TTAAGGTTTTGGGAAGTTTCACAGAGATGTCATACCCCTTTCTCTCCCTAAACTACCTTGCCAAAGTGCTAGTGAAGGAAGGGAGCA
CCCACTTTTCCTCCTGGCCAGTCTTAAGAGCTGGACTCTTCATGTTGTTGGTGTGATACTTTCCAAAGAGCACATAGTTGAAACAAA
TTAAAGAAGACCTAAGTAAATATAAAGATATCCTGTGTTCACAATTGCAAAACTTCATATTATTAAGGTAACAACACTCATCAAGTT
GATCTACAGATTCAGTGTAATCCCTATCAAAATTGTAACCACCTTTTTAGCAAAAATGGACAAGCTGATCCTAAAATTCATATGGAA
AAGCAAGGGACCCATAATAGCCAAAACAAATTTGCAAAAGTAAAACAAAGTTGGAGAATTCACACTACCCAGTTCAAAACTTAGTAC
AAAGCTATATAGTAATCAACATAGTGTGTACTTGAGTAAGATTAGCCATATAGAGAAGTGGAATAAAATTGAAAAATCAAGAAATAA
AACCAGGCCAGGTGTGGTAGCTCATGGATGTAATCCCAGCACTTTGAGAGACCAAGGCAGGAGGATGGCTTGAGCCCAGGAGTTCAA
GACCACCCTGAGCAACATAGCAAGTCCTTGTCTCTACAAAAAGTAAAAATAAAAAAATTAGCCAAATGTAATGGCACATGCCTGTGG
TCCTAGCTACTCAGCAGGCTGAAGTGGGAGGATTGCTTGAGCCCAGAAGGTTGAAGCTGCAGTGAGTCGTGATTATGCAACTGCACT
CTAACCTGGGTGACAGAGCAAGACCTTGTCTCAAAAAAATAAGAAAAAAATATATATGATTAAAAAGAAGAAATAAGGCCGGGTGCA
GTGGCTCATGCCTGTAATTCCAGAACTTTGGGAGGCTGAGTCAGGTGGATCACTTGAGCTCAGGAGTTTCAAAACAGCCTGGGCAAC
ATGACAAAACCCTGTCTCTACAAAATATACAAAAGTTAGCTGGGCATGATGGCGCATGCCTGTGGTCCCAGCTACTTGGGGGGCTGA
GGAGGGAGGATCACTTGAGCCTGGGAGGTTGCAGTGAGCTGAGATCATGCCATTGCACTCCAGCCTGGGTGACAGAGCAAGATCCTA
TCTCAAAAAAGAAGATGAAATAAGCTCGTACGTATATGGTCAGTTGATTTTTGACAAGGGAACCAAAACCATTCAATAGGGAAAGAG
CAGTCATTTCAACAAATGGTACTGGGAAAACTAGATATGCAAAATAATGAATTTAGACTAACTCACACCATATGTAAAAATTAACTC
AAAATAAAGCAAAGACCTAAATATAAGAGCTAATGCTATAGAACTCCAGAAGAAAACATAGGCATAAATATTTGTGACTTTGGATTA
AGCAGTGTCTTCTTAGATATGACATCAAAAGCATAAGCAATGAAACAAATGTAAATAAATTGGACTTCAAAATTTAAAACTGTGCTG
AAAATGATACCATCAAGACAGTGAAAAGATAACTCCTAGTATCTAGAAAAATAAAGAACTTTAAAAGTCAACAATAAAAAAGACAAA
CAACTTAAAAATGGGCAGCCAGGCATGGTGACTTGTCCCTGTAATCCCAGCTGCTTGGGAAGCAGAGGCAGGAGCCTGCACTGAGCT
ATGATCACGCCACTGTACTCCAGCCTGGGCAACAGAGCAAAACCCAGTCCCTTAAATTATTAAAAAACAAACAAACAAACAAAAATA
CGGGCAGAGGATCTGAACAGACATTTCTCTAAAGATTAAAATTGCTAATAAAGGCCGAGGCGGGTGGATCATGAGGTCAGGAGATCG
AGACCATCCTGGCTAACAAGGTGAAACCCCGTCTCTACTAAAAATACAAAAAATTAGCCGGGCGCGGTGGCGGGCGCCTGTAGTCCC
AGCTACTCGGGAGGCTGAGGCAGGAGAATGGCGTGAACCCGGGAAGCGGAGCTTGCAGTGAGCCGAGATTGCGCCACTGCAGTCCGC
AGTCCGGCCTGCGCGACAGAGCGAGACTCCGTCTCAAAAAAAAAAAAAAAAAATGGCTAATAAGCACATGAAAAGATGCCAACGTCA
GTAGTCATTAAGGAAATGCAATTCAAAGCCACATATTTCATACTCGTTAAAGCGGTTAGAATAAAAAAGACAGACAATAACAAGTAT
CGGCAAGGATGTGGAGAAAACGAAACCCTCATATGTTGCTAATGGAAATGAAAATGTTGCAACCTCTTTGGGAAACAATTTAGCAGT
TTCTCAAAAGTTAAATATAGTGCTACCATATGAGTCAGCAATTCTACTCCTAGAGGAGTACCCAAAAGAATTGAGAAAGTGTTTACC
CAAAAACTTGTATGCTCATGTTCATAGCAGAATTATTCATAATTGCCAAAATGTGAGAGCAATGCAAATACACATCAACTGAGGAGT
AGATAAACAAAATGGGGCATATCCATGCCATGGAATATCATTCAACCATCAAAAGGAATAAATTAATAGCACATGTATATGAACCTT
GAAAATATTTTGCTACATGAAAAAGACAGCCACAAAAGCCCCATATTATATGATTCCCTTGATATGAAATGTCCAGAAGAGGCAAAT
CCACAGAGGCAGGAAGTAGATTAGTGGTTGCTAGGTCTGTAGGGATGGTGGGATGGGAATGACTGCTCACAGGTACACGGTTTCTTT
TTGGGGTAATGAAATGTTCTGTGATTAGATAGTGGTGATAGTTGCATAGCTTTGTGAATATACTAAAAGCCCCTGAATTATACACAT
TTCAAGGATGCTTTTTACCACATATGAATTACATCTTAAATTTCAAAAATCAATGATTTAAAAAAATGGGACACAGTGGCCAGGGAA
ACAAGTTACAAGTGTCACTGTTTCACTCACATTTTCTTCCTTTCTCAGATATTTGGGATATGCCTGGCCCAGAATTTGGTTAGCGAT
ATCGAAGCTGTCAGGGCGAGCTGGTAGACCCCCTGCAACCGCTGCTGCAAGACACTGGACAGACCCAGCTTTCGGGACCCTCCCGCG
TGCCGAACTGATCTTCGAGCTGCATGGACCTAATCACAGATGCAGCCTGCAGTCTCGCCTAATGGAGCTGCCATTAGGGGAGTGTAA
AACTGGGAAATGCTGCTCACTGACAGAATTAAAAAAAAAAATAACCAGTATGAAAGTCGTTGCGCCGTGAATCTCTACTGTAGCCAT
GAATTTATGGACAGTTAGATGCTTACCAAAAAAGAAAAAAAGGGAGGGTAGGGGACCCAGATGTACTTGAATGTGCAGAAAATACAT
TCTTGTCCTCATCTTCCGTAATTGGAGGGCTGGGAGAGGCAGCTTTGCTCTTCACCACACCTTGGACGGACCACCTTCTTTCTGTTC
CATGGCCTGAAGGAGTGCATCTCCTCAAAGACTCAGCCCCTCACCTGGGAGGGCAGTGGTTTGTGGGCATCCCTCCATGTACATTTT
AGGAAACACTTGCAACTCTCATCTGAAGAAGAAAACAACTCATCTTTGGGTTCAGATTTTGTGATGGTATTCAGCAAGTCACTTGGG
CGAGCACACTTGGTCTATCCTGGAAAGTCTCCTTATAAGAGAAGTTGTGTATTTCATGTGCACCGAGCAAGGGCATTGGAAGACGTC
ATGAGGCTGTATTTTAGCAGGACTGATCGTTTTTCTAAGTAGACCTGAGCTTTGTTTATCAGTGAAATTCAAGGAGAAAATGAGGTT
AATGAAGAGCTATCAGTTAAATATCCCCTTCTTCTCACCCTGCCAAAATTAGCAGTTGGATTTTTGGAAACTCTGGAATATTCTGGG
TCATTTTGTTTTGTATGTTTGTTGTTTTTCGTCTTCCAAAGGTGAAAGCTATGATACAGTTCCACTTAAATTTTAGTGTTTTCTTAC
TCAGCTCAAGCATTAATTTTTGATTAAGTCTTAATCTGCATGACCTGTGAATCTGAATCCATCATCTCCCTTTCCTGCCAGCTTTTC
TACAAACATTGAAATATGTTATTTGGTCAGCACTTATTTCCTAGGTTCACAGCCTTGGGAGGTTGTGGCATGTCCTCCCAGTCTGCC
TGGGAAGAGACCAGCTGTACCATCCAAATGCTTCCCTGGTCTTGATGATCTCTTCCAGAGTCGATCTGAGTGGCCTTTTCTGCACCC
TCCCCTTCTTTCTCTTTGAATGGAATTAAACCCAATTTGGAAACAACATTGACCCAGTCAAAAGCTTCTAATGGTTTCTTTTTCTTC
CTCCAGTTTTAGTTTGCTTTTATTAAAAAAAGAAAATAGTGCATGGCCATAGCTCCTTCAGTTCTCTTATTGCAGACTAACCATCAG
GATGGTATCAAAGCACAAATACTTTGGAGGGGAATGCGTTGAACTGGGGCAAGTACTCTGTAACACAAAGTGGGAAACCACTTCCTG
GTGCTGCCGCTCCTGCCCCCACTTTAGGTGGGAGGGACGAGTTTTGCCCTCTAGATTTTAATCCAGCTGGTGTCCACCGGATGTTGC
CCTCCTGGGGAGCAGATATCAGTCTGTGGAACTCTGGGAAAACCACAGGCACATTTTTCGGTGCGGACAGATTTGCCAGCACATAAC
TGGGCAGCCAGCTAGAATACTTTGTGGAAATTAAGCGAGGTTTTCCATTTCAGCCCCATGGTGCATGGTGGTGGCCGATGAATGTGT
CAGTCTGCTCAGAGAAAGGACAAAAAGGAAATTATTTTCAAAACTGTGTTCACTGTTTGGGTGTGTGTATGGCTCTGCATGTGTGTG
TTTTTGTCTCTGTATAGGTAGAGGTATTCACATCTTACTCCGACTGTAAGGTTGTCTTACTTCATCTCTGCCCCCACCACAGTTGCC
ATTTTGTAATGTCCTTCCAACATGGAGAAGACACGAGCTCTCTCCAGTTGGCATCATTTGTCTTTTTTGTTGATTGCCTCATTCTCC
AGTGAACTCCATCTGGCCAATTGATTCAGAATCAGGCAAGATCCCTGCCCTTTGGCACATCCACTGAAAGGCCAAACAGCAAGTCCG
AGTGAGTTTTAAATATTAATTAATCACCCTTTATTTTTTACACTTGAGAGTGATTGTAATAAAGGCTGTCATTAATAAACTTGGTTC
TACCT
HUMAN mRNA SEQUENCE: AR28-002.1 (Seq ID No: 53)
CGCACGGACGCGGGCGCTCGCTTTGTGTTCGGGGCTAGCGTCGGCGAGGCTTGAGCTTGCAGCGCGCGGCTTCCCTGCTTTCTCGCG
GCCACCCCGGCTCCGGCGGCCTCGGCGCGCGAGGGGCTGGAGGTGCGGGAGCCGCTCTCCGCCGGTCGGTCCCCGCGCGGCTGAGCC
CAGGCCGCCAGCGCCGCGGCCCCGTGCGGTGTCCCTGAGCTCCTGCTCCCCGCCGGGCTGCTCCGAGCAACGGTGCTTCGGAGCTCC
AAACTCGGGCTGCCGGGGCAAGTGTCTTCATGAACCCAGAGGATGTCCGGGAAGCACTACAAGGGTCCTGAAGTCAGTTGTTGCATC
AAATACTTCATATTTGGCTTCAATGTCATATTTTGGTTTTTGGGAATAACATTTCTTGGAATTGGACTGTGGGCATGGAATGAAAAA
GGAGTTCTGTCCAACATCTCTTCCATCACCGATCTCGGCGGCTTTGACCCAGTTTGGCTCTTCCTTGTGGTGGGAGGAGTGATGTTC
ATTTTGGGATTTGCAGGGTGCATTGGAGCGCTACGGGAAAACACTTTCCTTCTCAAGTTTTTTTCTGTGTTCCTGGGAATTATTTTC
TTCCTGGAGCTCACTGCCGGAGTTCTAGCATTTGTTTTCAAAGACTGGATCAAAGACCAGCTGTATTTCTTTATAAACAACAACATC
AGAGCATATCGGGATGACATTGATTTGCAAAACCTCATAGACTTCACCCAGGAATATTGGCAGTGCTGTGGGGCTTTTGGAGCTGAT
GATTGGAACCTAAATATTTACTTCAATTGCACAGATTCCAATGCAAGTCGAGAGCGATGTGGCGTTCCATTCTCCTGCTGCACTAAA
GATCCCGCAGAAGATGTCATCAACACTCAGTGTGGCTATGATGCCAGGCAAAAACCAGAAGTTGACCAGCAGATTGTAATCTACACG
AAAGGCTGTGTGCCCCAGTTTGAGAAGTGGTTGCAGGACAATTTAACCATCGTTGCTGGTATTTTCATAGGCATTGCATTGCTGCAG
ATATTTGGGATATGCCTGGCCCAGAATTTGGTTAGCGATATCGAAGCTGTCAGGGCGAGCTGGTAGACCCCCTGCAACCGCTGCTGC
AAGACACTGGACAGACCCAGCTTTCGGGACCCTCCCGCGTGCCGAACTGATCTTCGAGCTGCATGGACCTAATCACAGATGCAGCCT
GCAGTCTCGCCTAATGGAGCIGCCATTAGGGGAGTGTAAAACTGGGAAATGCTGCTCACTGACAGAATTAAAAAAAAAAATAACCAG
TATGAAAGTCGTTGCGCCGTGAATCTCTACTGTAGCCATGAATTTATGGACAGTTAGATGCTTACCAAAAAAGAAAAAAAGGGAGGG
TAGGGGACCCAGATGTACTTGAATGTGCAGAAAATACATTCTTGTCCTCATCTTCCGTAATTGGAGGGCTGCGAGAGGCAGCTTTGC
TCTTCACCACACCTTGGACGGACCACCTTCTTTCTGTTCCATGGCCTGAAGGAGTGCATCTCCTCAAAGACTCAGCCCCTCACCTGG
GAGGGCAGTGGTTTGTGGGCATCCCTCCATGTACATTTTAGGAAACACTTGCAACTCTCATCTGAAGAAGAAAACAACTCATCTTTG
GGTTCAGATTTTGTGATGGTATTCAGCAAGTCACTTGGGCGAGCACACTTGGTCTATCCTGGAAAGTCTCCTTATAAGAGAAGTTGT
GTATTTCATGTGCACCGAGCAAGGGCATTGGAAGACGTCATGAGGCTGTATTTTAGCAGGACTGATCGTTTTTCTAAGTAGACCTGA
GCTTTGTTTATCAGTGAAATTCAAGGAGAAAATGAGGTTAATGAAGAGGTATCAGTTAAATATCCCCTTCTTCTCACCCTGCCAAAA
TTAGCAGTTGGATTTTTGGAAACTCTGGAATATTCTGCGTCATTTTGTTTTGTATGTTTGTTGTTTTTCGTCTTCCAAAGGTGAAAG
CTATGATACAGTTCCACTTAAATTTTAGTGTTTTCTTACTCAGCTCAAGCATTAATTTTTGATTAAGTCTTAATCTGCATGACCTGT
GAATCTGAATCCATCATCTCCCTTTCCTGCCAGCTTTTCTACAAACATTGAAATATGTTATTTGGTCAGCACTTATTTCCTAGGTTC
ACAGCCTTGGGAGGTTGTGGCATGTCCTCCCAGTCTGGCTGGGAAGAGACCAGCTGTACCATCCAAATGCTTCCCTGGTCTTGATGA
TCTCTTCCAGAGTCGATCTGAGTGGCCTTTTCTGCACCCTCCCCTTCTTTCTCTTTGAATGGAATTAAACCCAATTTGGAAACAACA
TTGACCCAGTCAAAGCTTCTAATGGTTTCTTTTTCTTCCTCCAGTTTTAGTTTGCTTTTATTAAAAAAGAAAAAATAGTGCATGGCC
ATAGCTCCTTCAGTTCTCTTATTGCAGACTAACCATCAGGATGGTATCAAAGCACAAATACTTTGGAGGGGAATGCGTTGAACTGGG
GCAAGTACTCTGTAACACAAAGTGGGAAACCACTTCCTGGTGCTGCCGCTCCTGCCCCCACTTTAGGTGGGAGGGACGAGTTTTGCC
CTCTAGATTTTAATCCAGCTGGTGTCCACCGGATGTTGCCCTCCTGGGGAGCAGATATCAGTCTGTGGAACTCTGGGAAAACCACAG
GCACATTTTTCGGTGCGGACAGATTTGCCAGCACATAACTGGGCAGCCAGCTAGAATACTTTGTGGAAATTAAGCGAGGTTTTCCAT
TTCAGCCCCATGGTGCATGGTGGTGGCCGATGAATGTGTCAGTCTGCTCAAAGAAAGGACAAAAAGGAAATTATTTTCAAAACTGTG
TTCACTGTTTGGGTGTGTGTATGGCTCTGCATGTGTGTGTTTTTGTCTCTGTATAGGTAGAGGTATTCACATCTTACTCCGACTGTA
AGGTTGTCTTACTTCATCTCTGCCCCCACCACAGTTGCCATTTTGTAATGTCCTTCCAACATGGAGAAGACACGAGCTCTCTCCAGT
TGGCATCATTTGTCTTTTTTGTTGATTGCCTCATTCTCCAGTGAACTCCATCTGGCCAATTGATTCAGAATCAGGCAAGATCCCTGC
CCTTTGGCACATCCACTGAAAGGCCAAACAGCAAGTCCGAGTGAGTTTTAAATATTAATTAATCACCCTTTATTTTTTACACTTGAG
AGTGATTGTAATAAAGGCTGTCATTAATAAACTTGGTTCTACCT
HUMAN PROTEIN SEQUENCE: AP28-002.1 (Seq ID No: 54)
MSGKHYKGPEVSCCIKYFIFGFVIFWLGITPLGIGLWAWNEKGVLSNISSITDLGGFDPVWLFLVVGGVMFILGFAGCIGALRENTF
LLKFFSVFLGIIFFLELTAGVLAFVFKDWIKDQLYFFINNNIRAYRDDIDLQNLIDFTQEYWQCCGAFGADDWNLNIYFNCTDSNAS
RERCGVPFSCCTKDPAEDVINTQCGYDARQKPEVDQQIVIYTKGCVPQFEKWLQDNLTIVAGIFIGIALLQIFGICLAQNLVSDIEA
VRASW*

TABLE 6

MOUSE GENOMIC SEQUENCE: mD28-010 (Seq ID No: 55)
GCATTTCTTACTTACTGAAAGGTAAGGGTGAATTGTCCTCAGTATTCTTCCAGAAGTTTGTAAGAATGATAACTTAGATGATGGGTG
GATGGCTTAAGAAGGGCTGATAAATGTATCCCTTGAGGCCAAGTGTGTACAGCTACAAAAGGTGCTTGTCCCTGGTGTCCTTAGCTT
TTGGGGAGGACACAGTTCCTTTCTCTAATAGTTATTTCTCATGAGTCTGTATTTCAGACACTCACTGGATGAAAGTGGCTTTTGGTC
GAACTGAACTTGGAGTGGAACTAAGGGAACATTGGGTTCTCTGAGGTGTAAATGAGTGTGAGCACCTGTCATTAGGGATCAGCATCT
AAAACTGGTCTGAGAACATTTCCAGAACTATGTTGTAGGAATTGGAGTTAGAATCCTAGTCTCATAAGTTGTGGTGTCTCTCACATG
TCTTTAGAGATCATTCAGTCTACTTTTTGGAGGAGACAAATCCAGTGTTGGATTCTTAACACTGTCTGGAACGTGGAAGGGTTGGCA
CAGAGGAGAAAGGAGACTTTTAAAGAAAGCACTAGAAGAAGAGCGAAAGGGAGACCAGTCCCACCTTGCAGGTGGGAAAGGGAAGTT
GCTGTGGGGGTGGGGTGGGGTGCTTCTGGTTCCCTTCATAGCAGATGAACTGTCTGCTAATGAGGGATTCTCAAGATATCATATGCG
CACTGGGAATCTTCATAGTTTTGGCCATTGCCAGTGTGTTTTGATTACAGGTGACTTTATAGAAATGACACAGATGCTCCCTGTAGT
AGGGTGTACCAAGCAAACTGTTTGGGCAATACTTTCTTTCATACTTTTCTATAAAAATGTAAGTAGCAAGTAAGGCATTGTTTAAAA
GAAGAAAGTGCACACTGGGATAACAAGAAAAACAGTCAACATTGAGGTGTTCTAAAGAAATAAAAGGACTCCTTTTCTGGAGAGTCT
ACAGTTAACTGGGGGACATGCATTTTATGAGTCTTTGGGGTTGGTCAGTTGGGTTGGGCCCATTTCATCATTGATTGGCTAGAGCAC
ATGAGGCTGTGCACTTATGTTCTCTGAACCTTAATCTTTTTCATTTAGATAACAGATAATCTGCCCAAGTGGGTGACCATGAGGAAC
AAATGCATAGGTAAAGTGCTTGCTGTAATGGTAGCCCCTCAGTGAAGGTTGGCTTTCTTTTTCAGGAGAAAGTCCAGATACTGAGTC
AGGTCAAGCTGAGGTGTTACTGTTTGCTCTGTAAGAAAACAAAAACAAAAACAAAAACACCCGCCTTGTGCTTATGATTCTCCTGCA
TGTTAGGATTATATGTGTGTACCACCACAGCCTTCTAGAAAGATCATTNNNNNNNNNNNNNNNNNNNNCATCCCCCCAAGCTATGTT
AAGAACATGGGATTGGACATGATAATAAGAATGATAAGATGATTGGGTTCTGATGAATAATATTCAAAAGTCAAAAGTCCCCCAGAC
AGTTTCCATATAAGTCTTCAGGTTTGAAGATCTGAGGGAAGATCTGAGCTCTGAGGGAAGTCATTGCTGTTGAACATAAATACTGCA
TTTGCAGTATTTTCTTCTTACCAAGGCCAAAGTTTGTCCTGTGTCCTACTGTCAAAGGCAGGATCCAGAAAGAGAACCTGCTACACT
TTTGTGTTGGGAGAGGACTCTGGGAAGCAACCCTGTGCATCCTTTTGTTCCTGTCTCAGAAGGTCCACGAATAAATGCACATATGGG
TGTGGAAACACTTAGTCATTTGTTGTCATAAAATTGTGTTCATTCAGAAACTTCTGGTGTTTAACTTAGAATGGGTCTAGCCTTTCT
GTGGCCAACTCATTGATTCTTAAACAATGGAAGATATGATGAATTTTCTTACGTGTCTGACCCATCTGTCATTTGTAAGTTGTGATA
ATGAACATGAATATTTACTTCAATTTGAGGCATATTAACTTACTACAATAAGACAGTCACCCAGTGACTCAATTATTAATTGTATTA
AAATATATATTTTCATGTACATAACACTTAACAGATTAAGCTATATTTCCTAGTTGCATAGGTGGTAAAGAAAACCTTAACTTTTAA
AAAAAATTCTCTAATAATGACAATATGAATTTATCTAAGATATCTAGATTCGATACTTTCTTACCATATTTAAAAGGTTATCACTTG
TTGGTATGATTAAACCCAGGGCCACAATCATGGTAGGCAAGTGCTACATGCAGCTCTGGAGTGAAGAGAATCAGAACTCTAGGTCTG
TGTTTTGGAAGATGTTTAAAGATGAACATGTTCCTCACATGCATGCTGGCTACACTGTGCTTCCGTCTTTGATCTCTGATAGAATTC
AGTCAGTAAAGCTTTTTCCTGGGCTTAGTTTCCTGCCTTGTATGATAAGGGGATCCACACCACAGGATTGTTGAGTGTAAATGAATA
GATTTTCTAAGGTGCTTAGCTCTAGCAAATGTTTAGTAAATGCAAGCTTGGAAAGACATTCGGTTCATTATACACCTTTAAGGGATA
AGGTGCCCTTTATTTGGTAGAATTAAGGTAAGTTCATTTTTGCGTCCTGTCCTCTGATGCATAGTCATCATCAAGGTTTAATTACAG
GAAGGAGAACGTTAACTTCTTGTTCTTCAGCAAGATTGTGATTACAATACTGCTTCAAAATGACCAGTGTCATAGCAAATGGTGACT
TTTTTTTTTTTTTTTTTTTTTTTACATCCTTCAATGTCTCAGTCCTGTGGTATAGAATGGAATTTAGATTGATCTCCTATTGAATTT
CTGGGGTTAGCAAGTGTGATCATATTATCTCCATGGTATGTTTAACTTTACATCTTCATGTACATGAAGAAATACTACTCCATTCCC
AAAGAGACTGTCACAAAATTTTTAATCCACCTTTTATGAAAAACAGCAATCCTTATCCTGCTCTTTAGGTCAGAGAGGACCCTCATG
GTAGTGGTGTGCTATATTTGGAGTCCTCCTGCATCTCTCCCAGGGCACCTGGGCTGTGAGTCTGCTCACATCAGGGAATGCAGACCT
AAATCCTGTCTCCTGGGGTAAGAAAGGTGAGAAGCTGCTGCTATGCCCATATCAAAGCCCCCAGCCCTCTGAGGACTTGGGGCATTT
GTCCTTTTGGGGCTAGAATATTAGATACTTTATACATCAGTTACTTTTCTGTGATAATATATCATGGCCAAGACAGCTCAGAAGGCA
GGGTTTATTTGGGTTTATGGTTTCAGAGGGAGGGTCCATAGTGTGTGTTGGGGGGCGCACATCTTCAAATGCAACCATGAGCCAGAG
AGTAAACCGGAAGTAGGGCAAGGCTGTGAACCCGTAAATCCCACTCTGGTGTGCTTCCTCCATCAAGGTTGTATCACCTTCCCAAAT
TATACACCAACTGGGGATCAAATGTTTGGATACCCAGAGCTCTGGGTTTCTCCTTCAAATCACTACAGCTAGCCATCCTGTGAGCCT
CTGAGTGGAGGGCATTCCTTCCCAGTCTGCCCAATCCTTGTCCAGTGTGTGGGAGTGCTAGGTAGAATGAAAGCCAACCAGGAAAGA
ACATGACAGTGGTTCTTAAAAGATTCCCAAGAGCTCCTTCTTTTTCTTTAAAAAAGATAATTATGTTGATAATTGCTATTGCAATTA
ATTATACAAAGCTAAATAACTTTAAGTTATCCAGGGGAACTATGTATCCTCTTGTGAATTCATGTATCCCATCATGAACGTACTGAT
AAAATAAAACCCTTCAATTTTGGATGCATAAATCCAGATGCTCGTATGCTTTTCCTCTGCACCAAGAAGCTTATATTTTTGGTGGGG
GGCCTGGGGTAGATGTGTGGCTGTCGGTTCCCTTTCCTTCCTCTTCTTTTCTTTTCCTTAGCTTTAAGAACACCAGACTGCTTCTCA
CCTCAGGGGGAGACATCAGTCTGCAGTGTGAGGAAGCAGACCTCATTTTCTGCCTGGGTGCTTGTTTCTAGCTTATTAAGAGCTGCT
TTAATTGGGTCCCTAATTTGTCAGGAGTACAAATTGGTCAGCTCCTACTTAAGCAATGTAATTGGTGCCAGTGATTTGTGAGCCAGA
GCAGAAGGATGGGCTTGGGTGACCTCGGCATGGCTTGGTGAATAATGGTGTGAGCCAATTTTTAATCTGATCTTTATTCATATCTGG
CCTTGCCTTAGAATTTATGCTGCTCTTAGCAATATACAATGCCGCTGTAAAATGAATGACACACAGACTATGAGGCACTGCTTAGTC
CTGGTGACATTGTGAGTCCAGTCACATCCTTTCTTGCCCACACTACCCTGTAATAAACTAATTTTGTGTCCTTCTTCTCCACCTCTC
GTGCTTTGTGCATGGCTCATTCCTTGGTGTTGAATGAGTTATTTCAAAGCCCATTGGTGTCCCTCTTATTTCCATATATATATATAT
ATATATATGTGTGTGTGTGTATGTAGTGAGAGCACAGGCCTGGGAAGGAACATGGCTATGAGACCTTGATTGGCCTCGTCTCTTAAC
TTCTCTGACTTTCGCTTTTTGATATTTCCATCTCAAATGCATGTGCTCTTTGCTTCACAGAGCTCCTCTGGGGGGGGGGGGAATCAT
AAGATAATTCAATGAAAGGCTCAGCACTGTGTGGCTTGTTTCCAGTAAGTGCCGCTTCACTGCCTTTGTGCTGTGATCCTGTACAGT
CTGCTGGGACATCTGTGACTTTCAGGCTATGGTGGACTGAGTGAGGGCACCTTCCCAAGAGAGTTCCACATTTGAGGTTAAGGTCCT
GATAAGCCACCTGCTGGGTAAGGTGACTGAGGTCTTCTCTTCAAAACTCATTAAGAGCTCAGAGCCTTTGATCTTTGTCACTACAGG
TAGCTTGGCCTTTCAGATAGTCACTATAGGAACAGCCAGAATTGGTCTTTGTACCTGAGGGAGATGCTGACAAAAAGCAAAATTTCT
AAGCAGATGGTTGGGTCAACCCTTCCAGGAATCTCAGAGGAGAGAGTATGGAGTTCTCTGAAATGGTCAACTGAGACCTGTCCAGGT
AGTGTCTCCAAGTTGTCCTGTGACGCAGGTGTCCGGTGGCTTTAGTCAGCTTAAGTTTTAAACAGTGGTTTCCAGTCAGAGTCCTCA
TGACCCGTGGCCAGATACTTACCTAGTGAGTCATGTTTCCATGAAGAGGAAGTGAAAGCAAGACCTGTCTGAGAGGGGCATAACTGT
ACCTAGCATTGGTGAAGTTTGCTCTGAATCAAGGCTTTGCCTTGCTGTTACAGTTGTGTGTGTGAGTGGGGGGTGGGGGGGGTGGGG
TGCTGTCAGAAAGTGGGCTGAAAGGAGACATCCAGAACAATACTTTAAGTTCTGACTGAGAAAGTGCAAATTGTGGCTAGTACATGC
TGTCTGATATTAACGTCACTCTCAAAGATGGATACTCTTCGCTGCCTTTTGCTTTAAGGAGAGTGACCAGGCCCCTAAGTCAACACA
GAAGCCTGGCCCCAAACCATCAATCTTCAGGGTCCATGCTTCTTACAACTTCATAGTGTGGAAGATTGCCCTCAAACAAGTCATGGT
ACCCTTTCAGAAGATGTGTCAAGTGGTTATTGTTATTTCCCACAGGAAGCCAGCCTATGAGTGAAAAGACCATAGGCACATGACAAA
GAATTGGTGAGACCATGGAGTTCTCTGAAATGGTCAACTGAGACCTGTCCAGGTAGTATCTCCAAGTTGTCCTGTGACACAGGTGTC
CGGTGGCTTTAGTCAGCTTAAGTTTTAAACAGTGGTTTCTGGTCAGAGACCTCCTGACCCAGATACTCACCTAGTAAGTCATGTTTC
CATGAAGAGGAAGTGAAAGCAAGTTTTGATTACTCTGTGTTTGAACCCCTGACATAGACCTTCATTTTGTTAATGTTGATTTCTCTG
TCATTTTAACTTTTAATTCTTTTTTAATTAGATATTTTCTTTATTTGCATTTCAAATGTTATCCCTTTCCTAGTTTCTCCTCCAAAA
ATCCCCTATCCCCTCCCCCTGCTCTCCAACCTACCCACTCCTGCTTCCTGGCCCTGACATATTCCAACTGTTTCTCAATCATTTATT
GGAGCAGGACTCTTACTGCATTTAACAGTGAAATAATCCCTTCCATCAATTAAAAGAAAGAATCAAACTTTCAAGATTAGGCTTTAA
ATCTCAAATTAGTGAATAGTCATTTTATGAGAGTAATATCTATTCTGAAAGCTTATAGATGCGAACCAATTACTCCGCTTTCCATCC
ATATTAGATGATGGTGGATGTAGAGCTAACAAGCATCTGTTGAGATCTTAATACACGTGAGCTATGGTATGGCGCATTCTGGGAGGT
TAGAAAGTAACGAGGCCTCTATTCCACAGGGTTTTAGTTTTTAGTAGAGACAGTAACTCTGATAAAGATAAGTAAATAAGTAAGTAA
AGGATAAAAAATAAAAGATAAATAATAAAAATAGGAAGTTCAAAAGGAAGAGGCTCTGTTTGGAGATCAAGGATGACTTCCTGCAGG
AGGCTGGGACAGGGTCTAGCTCTGCAGTGGCAGACCTGTGCTTCTCTTTGGGAGCTGAGTTCCTGTCTGTTTAATGTAAGAAGTTTC
TAAGTAACTGGGTGAGCTGATGGGTGGAAAGCAGAAGCAGTTGTGAGAGAAGAGCTCCCAGCAAGGCTTTGTTAAACAAATACCTGC
TGCTGTGCCCCAGGCCTGTGGCAGCTGGGGGAGAGCCACTAGTACAAGACACAGATCCAGTGATCTGGGCAAAGGGCACAGTGGCCG
GGTGTGGGGAGGGAGTGGCAGATAAGCAAACCTAAGAGAGTTGGCAGAGCTTGTGAAGATCCTGGATGCTGAGGAATTTGGGGAGTG
GGACCACCAACGGCTCCGTGCTGCTCTTTTATTTGGAATAAGAAACTATAGGCTGATAACTGATCATGTGGATTTCGCTCGTTAGAA
AGGTAGTTAAGCCTAAGCATAGACATTCCTCTGTGCACTTGAGGATCCTGCAGTGTCCAGGACATCTACTGATACCGGCTCTGTGCT
GCTCAAGGCCCTTAGGTAAAAGGGTATGAACCGCATGCACCCACTCATCTCCTCATGCATGCTCTGAACCAGCTATAGATTACATAG
CTGCATGATTGGTTTTAATTTCAAAGTAATTAAGATGTAAGGTTAGCTCCGGCCGCTTTCCTCCTTCTCCCTGTGCGTTTCCTCCCC
CGCTTTCCCATTCTGTGTATGACTTTGCTCTATCGCCCACATCCCTCAGGATATCTTTCTTCCCCTCATAATTGTCTTTTCAATTTC
CTGACTTGTGAACACAGATACAGAAACACGTACGTATATATTGAATTTTGAGGCTATGTTCAGCATAGAAAAGCAAACATGCAGCAT
TTGTCTTCTAAGCCCTCTGTACTTGGTTTTGCATAGTGATCTCTGGGCATCCTTTCATGGTAATCTTACCTATCGTTCAGTACCACA
CCATCCTCCTGAGCTGTCTCACCAGCTTAAATACCTAGTTCATTGTAAATGCCACAGGAATCAATATCATCTTTTCTTGTTTGGGGA
ATGATAAGAAATAAATGCCCCATGTGTTTAGTGCTGACATATGCCTCCTGTCCTTATCCCTCATACACACACACACACACACACACA
CACACACACACACACACACACAGTGTGTGTGTGTGTATATATATATATATATATATATATTCAGTGGCTTGATGTAGCTACAGATTA
GATTTTGTGCCTACATGGGGCTGCTCGTATTTCCTCTCACAATATTCTAGAATATTCGATGGCTTGTAACTTGTTTCAGTGTTTTCC
CAGAGCAAATGAGCGGTGCTCAATAGTAGAACACATGTCTCATTTGTATTTAATTATAATTAGTTTAATCTAAAATTATATTTTACA
GGTCTGCCAAATTTATTCTGTTATTATAAAAACTGAATTTTAAGCAGCCAAGGAATTAATGAATCTAGGGCTTTAATTCTAGCATTT
CCTTAAACCAGCCATGGATGATTGGAGTGAGCTGTTCAATTTAGATGTCATATGTGTGAGAACTGTCCATTGTTCCTACCTATGTGG
CTATGACTGTATCCTTGCTACCGTCACATCACATAGACATAAAAGCTGAAAGAAGCTTTGAAGTTTTGAGTCCATCTGCCTTGTGAG
ATGTCTGACCATGCCACCTCTTAGCCAAGGGCCTTTAGGCAATTTGCTCAACTTTGAATCTAACTTCCTTATCTGTGACACCGCAGT
GACAATTCCCTATGACTCCTTATTACATGTTTATTATTTAGTAATCACTAGGCATTTTTTCAAATAAGGACACTGGGGTGTGGAGCA
ACGAAATTCTTCACTTATGGTTGAAACAGGGCCGGCTGGGCTTCCTGGCTCTGTGGCACTGTCTGCTCTGCTCTGTGGTGGCACTTT
AAGGGCTTTTCAACCAGGGGGTCTGAAGCTGACACATGCTGGTTTTGTCTCCCAGGGTTTTTGTGAGGCTAAAGTGAATATCACGTA
CTTCTTATTAAGGGCATTTAAAAGTATTAAAGTTTTAATTATTTTTCTATGCTATTGACATTAGTGATACATATTCAATGTAGAGAA
CTTTAAACAAATGAAAGAATGAACTAAGGCGGGGCCCTCCATAATCCCTAAGGATAATAATAAACTGGCTTGGAATTTTAGTGAAAT
GAATACATGCTTTGGTTCCCTGTTTGTTCTTTTTAAATTTTATTTATTTTGACTTTGGCAGCTGCATCGCCTTACAGGCCAAGTGTG
AATTTTATTTACATACTGGAATCAGTCATGACACATGCCAGGATGAAAGTTAAAATCTCTGTGAAATGTAAAGCCTTAAAGGGGCGG
GGAGCTAGGGAATAGTGTGAGTTCTGGGGCATTCAAAAGCACAGGCTGTAATAATACTCAGAAGACCAAGGGGCAGCTGGCGAGGTC
TCAAAGGCTGTAGAAGTTCTTGGCTTTTCTGCACTTGTGGAATCTCACGTATTTTCGGCTTTGAGTCAGAACCATGAATTTGAGTCA
GAACCTTGAAAATGATCTGACCTAGCTTTGCTGGCATCTACCACAACCTATGGGAACAACTAAAAATCATAAAGCAAAACGCAGAGC
AAAGATGACCCAAAGAGATCTGCTCTGCCTTTGTTCCAAGGCTCTGAAGTGAGTTGATCACAGATACTGCAGAGCTCGTTTGGAGAT
GCTGACTGGTGCGTTGCTATAGATGGGGAGCGAGAAGCTTCTGGAAGCTCAGGCATTCCACACTGTCTACCCAATAGCTGGGGCCAA
TAGACAGGGGAGAGTTGGCCTCTAAGGACTCTATTACATTAGGTTTGTGACTAGAGGTCTTCCAAAGATTCTGACTGTTGGGAAGAT
GTGACTTAGCAGGCTGGTAACTAACACGCTTTATCTCTGGTTCACTCACAGGAGTCCAAGTGAAGAGAGCAAGATGGTGTCTGCCCA
GCTTCACTTCCTGTGTCTTCTCACACTTTATTTGGTAAGCACAGAGGGTCTCTGCAGGATATTTCCTGACAGCCTTCCACTCCATAC
CATGAAAAGAACATTTATTACCACTTTTTTAAAAGGTGGTTTATCAGAATTGTACAAAAAGATGTGACGTGTTCTCGTATGATGTAT
CCAGTGAAGCGACCACAAGCCCCACGGTTGCCATCGGCTGAAGGGTGCACTTTATCCTCAGCCCTTAGAAGCTAATACGCCGTGGAA
TGGCAAACTGAGCTCTATGGTTATAGCTTTACTTGTTTCTGTCTCTACCGCTGTTACCTACACCCCAGACCTTGTACATAGACAATC
ACTCTGCCACAGAGTTACAGCCAGTACTGAACAAACCCACCTTTCACCTGTAGCTTCTGGCCATAGAAAAGAATTACAATGATTTAT
CTGTAAGCCTCTATAACCCAGCTTTAAACCCACAAAAGCACCAAGAAACTATTTTTTCCCTGTTTTCACCTTTCTATGCTCTGTATT
TTTATTTTCATAAATGGCTTGGAGGTTCCTAGTTAAATTTGTCATTAGCCACATCTTCTATTGTTGCTATTGTCTGACAGGCTTTCA
GCACGGAACAGAATGGAAACTGGGCCCCAGCTATTGGGTAGACAGTGGGGTACCATCTCTACTTTACTCACAAGGCTCCTGTGGTAC
AGAAGGATGTCCAAAGTTTCAATGTCTTATGGACCAAGATCTCAGGTGTGCTTCTGCTTGTTTCCATAAAAGGAAGTTTTAGATGGC
TGTACCTAAAATCTTGCTTCCTTTTTTCTTGTACCCTAAAACTACACACCAAATTTTAAGTCCTCAAAGTCCTCCAAGGGCCTTATA
GTTTAAAAAGGTTTAATGTTTTTTATTAAAATACATTTTATATGTAATGCATATGTACACCTTAACACATTACAAAACGTACTTTTA
TCACCCTACCCTCTGTATCATAGAACTGAACCAAGATCTCTGGAGCTTCCATCAAGGCTAAGGAGGAGACTGCTGTTCAGATCGTAG
GAGAATTCTCACAGCCTGGGTCTGAATGAGGACAGTGCTCTCCCTGTCTTTCCTGCCTACGTTATCATAGGAGGACAGTAACGTCAG
TGCCAGGTTCTAGTTTGTGGCTACAACTTGCTCATGATAGGAAATGGACTGAGTAGCTTTAAGCTTTAAATCTCAGTTTTCTGTCCT
TTGGGCTGGGTCTCATCCTAGCATTTGACATAGAGGAACGTTTGCATCAAGGAGTAGCACAGCCTAAGCTCTGAATACATAAAAGCC
ATTAGTAGTCTATATTTCAAATGTCCAGCCCCTATATCCATATAGACCTGCCCTCGATAATGAATGAGGTCACCTTAACAGGCTTTT
CTCAGCACCTAGCATAGAGATAGGAGGTGATCACATGATCTCAATTAGGAAGCAGCGTTGAGATCAGAGAGAATGAACATTTAGCTG
TGGTACTTCGCTGGTGGTGGGCTCATTTTCTTCTTGTCTGAGGTGACTTGCCTTCCTTTCTGTAGATGGAAGCTGTCTTTCTGCCAC
CAACACCCTTCTCAGCTCTGGCCGTGGTGGTTTGGTGACTCATATTGTTCACATTCAATTTGAAATCTTTATAGCCCCAATAGTCAT
GTTAATGGGAGGGAAGAAAGATTTGTTGCTCGATACCAGGAGGAGGAGGATGTAACTTTGAACCCTGGAACTGACTCTCCCTCCCTG
CATCTCGACAGCTTGTCTGTATGCATAAAATGTAGCCTTGTCTGTGAGGTCTGTGGGCTAAGGAAGTTTCCATTGGTGTGGATAATT
GCGAGGCTGATTGCTCACTGGGTGGTCTCAGAGCCGAGCCTTGGAGCTCAGCTCTCTGGTAGCACTGCAGAAAACACTGTCCTTGTC
TTAGATCCAGCTACACTGGAGGCCTGTGCTCAGCTCTCCTGCCTCACCTTCTAGAAGTAATGGAGCTGGAGTAGACCTGAGAAGAAG
GAAGGAAAGGCTCTTGAAGGAAGGCAAACTCAGAGAGACGGTGCCTAGAAGTCTGGAAAATTATTGTGGAAGTTAAAAATGCTTTTA
GCTGCAAATAACAGGAAATCGGGATTTGGAGCAACTTAAATAAATGAAGGGTTATTTCTCAGCGATAACAGGAAGCCCGAATGATAA
ACTTAGGGCAGGGCTAACCATTCTGTGATATATCTGAGGGGGGATGCTTTTGTCTTTGGACTCCTCACTGCTCTGGGTGACTGCTCC
AGCTGCACATACAAAGCCCGTGGCCTTTCTAAGAACAACAGGGAGGGGTAGAAATGCCAGCCATAGCTGCTCTTTCCCTTTAGGAAG
GAAAGGCCAGCATTCAGATTATGACATTGACTTCTGGTTCTGGGTTTAGACGCCTATTCACCTGTTCTACTCCCTTTGGGAGTGGTA
GTAAGAGTCAGTAGAGATGGCAGCCATTGGCTATGGAGTATTCCCCAGGGGTTGGAATGGTGGGAGCCACACCTTCCGGCACCTCCC
TATTACAGCCAACTTTCCTTCATCACTTTTGCATCCCCTGGTGGACAATAACGCCTGGGGTTGAGGAGGTTGGTTCCAGACATGCAG
GTTCAAATCCCGCTTCTAGTTACTCTGAGGGGGAAATAACTTTGCTATGCCTTAGTCTCAATGGGGCTAGCTGTACTATACTTTCCA
TAGTATGTCGCAGAGTTATTGCTAGGATTCAGTATGCTTAGGAAAGATTCCCATGAATACCACCTTAGCAATTATAAACTTTCCTTA
CCACCCTGGTCTGGATTCGAGTAACTGCTGAGATGCTTAGATTTCTAAGGTCTTTAGGCTTTAGAACCACGTGAGACCACTGCTTGA
AGTAAACACTTTATGATTTTAATTTATAAAGTGTTGGTCAGTTGTGACGTATATATGCTGAACTTCTAGTTTGACCGGAATTTACCA
CGTGATTGTGTGCCAAGTAAGCATTGTGCTAAACTATCTACATATTCTTTCTCCCTCTGATAAATTCAACCGCCTTTGAGGTGACTA
TAGACTCCAGGATGCTGGGGACGTCTCACAAATGTCACAGAACATTTTAAGTGGTGGGGGTATTTCAAAAAAGCTGGTATAACCATG
TATAATTTATAAGGCAGAACGGCTCTGAGAGGCTGCAGGATTTTGTTTAGTTAAGACAGAGCATGTGCTGTCCCTGCTTGTCACACC
CATGGTTTACTCCGGGTTTCACCCTCAGAAGTCTCCTTTGTAACATGGGAGTTAAGATTCTTCCTATGGGATGGTAGCACTCATGGC
TAAATGAGAAAAAGCAATTTGAAACTCATGGCAGACTACTTTGGCAAAGTAGGAGAGTCTCTCAATAAACACTAGGCCAGTTTGCTT
TCTGACCAGGTCCACACTATTTTTGGCCATCATGGAACCCTCCACTCCGTACATTGTTTCTGCAAGTGACACCCATCCATTCACACA
CGCTCTCTTTATTAACAGACCTGTGGATATGGCGAAGAAGGGAAGTTCAGCGGTCCCCTGAAGCCCATGACATTCTCCATTTTTGAA
GGCCAAGAACCGAGTCAAGTCATATTCCAGGTAAAGACTGATCTTTGTTATCTTGATTTCTGTTCGTAGTGGACCTTTGCTCTCATC
TCTACTGAGCAGCAGGGAGTAACACGAAGGCGGGCAGTTTTTATCCTCTGTGACTTTGATGAAGCTGAGTTGCAGAGCAGTGTGGGT
GTAGCATAGGAGGAGGAGCTAGCATTCTCACACAGGCTCCCTTAATCACGATTGTGGCCAGTGCTTCAGTCCTCTCAGAGGAGGTCG
GCATTATGGACTCCACCTCTGTCCTGAGTGCAAAAGCTGCTGATTTCTTCGGGAGGTGCTGGCTTTCTGGTCATTAGGGCATCCGCT
CCTCACCTCGCTTGGCCTGTGAGGTGTTTGAAGTGGGTGGAGGAGCTTCTCTGTGAGACCACAGCCTGACTTCCTGCCTGCCTCCTC
TGAATCCTTGTCGATGTTCTTGCCTATGGGTTGCCCAAGTCATGTTGGACATGTTAATCACCATTTCCATTACTGTGATGATTTTAG
TTTTATTTTCGGTCGCTGTGATAAATACCCTGACAACAATCAACTTACGGGAGGAAGGGTTTCCTCTGGCTCACGGTACCGGAGGAT
ACAGTCGGGCATGGTATCGAAGGCAGGGCAGGAGGAGTGAGACACTGCTCAAATTCCGTACATGCTCTGTAAGCAGAGAGAAAACAG
GAAGTAAGGGTCAGGCTCTAAAACCTCAAATCCTCTTTCTAGTCACCCTGTGATGTGCGTCCTCCAGTGAGCTCCACCTCCGAAAAA
ATTCCAAAACTGGGCCACCAGCTGGGGCCCAAACATTCAAATACAGGAATCTGTGGGTGGCATTTCACTTTCAAACAACAATAATTG
CAGACTACTGGGTGTGGAAGGAGAACGACTCAGAGAGAAAAAAGTTTGACTCATGACTTCCATCCATGGTAACTAGGCTCCATTTTC
TGAACCTTTGCTGAGGCAGGGCACGGTGGAGGAAAGCCAAGGCCTGTGGTAGTAAGAAGCAGAGAGAGCAGGAAAGGTCCAGAGATA
AGCTATGTATTCTAGAGGTACTCTCCTCTAGAGTGACCCATTCCTCTAACAGCAGTTCTCAATCTGTGGGTTGGGTCCCCTTTGGGG
GTCCAACGATCCTTTCACAAGGTTCACCTAAGACCATGGGCAAATACAGATATTTACATTATGATTCATAACAGCAGGAAATTGCAT
TTATGAAACAGAAACAAAAATATTTTATGGTTGGGAGGTCACCACAATATGAGGAGCTATATTAAAGGGTCGAAGCGTTAAGAAGAT
TGGGAACAACTGCTCTAAGTAGCCCCCACTTCCTATGATCCCATTCAGCAAATGAAGTCATTAAAGCATTTGTCTCCTGATGATGTT
CACACTCTTACAATCCAATTCCCTTAACGGCATCACCAGATGGGGACAGGACCTCCGATACATGAACCTTATTGGAGGCAGACAACT
CATATCCCAGCCATAATCTTGGCAGGTAGTTAGGTACCCATGAAAAGCAATAGGGGCTGGGGAGATGGCTCAGTTGATAAAGTGCTT
GCCACACCAAGCGTGAGGACGTGAGTTTGGATCCTCTGCGCCCACATATATGAAACACTAGATAAAGTGGCTTGTACCTATAATGCC
ATCACTGGAGATACTGAAGCAGGAGGACCCCAAGGGCTTGCTGGCCCAGCTCTTCTTGCCCAGTCAATGAATTCTACATTCAGTGTT
ATATTTCTGCCTTGTAACAGAATGGGGAAGACCTATGAGGGTAGCTCAGTGGGTAAGGAAACTTGTTCTGCAAGAAACAAAAGACTG
AATTAGAATCCTAAGCACCCACGTAAAAGTTGGGTATTGGTGCTTGTAACCTTATCACTGAGGGAAGAGGTAGTCAGATCCTGAGAT
TGCACTGATTGGCCAGCCTACTCCAAATAGCAGGCTCTGTATCAGTGAGAGTCTCTGTCCCATGGCAAATAGGCAGAGAGTAATAAT
GGGCAAAGACACCCAATGCTCTGTTGTGACTTCCTCGTGTGTGTGCAGGGAAATTCAAGCTGTGTACTCACACTCGTGTACCCATAC
CACATACAACATGCATCACATGAAATAAATGTGGAGATTAATACAGACTTCTGATGTTAAACTCTTACCACATACACACTCACACAC
TTGAGAACGTGTACACACGCATACACAGAACACATAAATATGTGTATGCCATACTTTTGAAGGTGGAGTGAAGAACCTTGGCTCTCC
CCCAGGGGGCCACTGACATGCATCAGAAGGAGATGACATTCCCTACAAATGTACCCGAGGGTCCAGGCAGAGCATCCTCTCTACATG
TGTGCATCTGCCTTCTCTTCCCACCGCAGGCATCTTTATAACATGGGACAAACGGCAGTGACCTTGCGTGGGAATAGTTTGTCTCTT
AGCTGCTCTCAAGGACACAGTGCTTTTTCTCCTGTGCTGTTTTGCTCCAACAACATGCCTTAGAGGACACTTGAAATATCCAAGCTG
AAACCTGGGACAGGAGACCTCCTTGCTCCCAGAGCCCATGTTTGACAAGCTTATACATCACAGGACAAGTCACCATTGTACAGTACA
AATGGGCTTGACTTAAGTTTCTGGTCAGAAAGATCTGTCCTTTGACTGAAAGACAGACAGCTAGGGAGATTGCAGGGTCCTGGCTAG
GGGGCTGCAGTACAAGTCTTTCTCCTTTTCTGCCCTGCCAGGGAGCTCATGTCAAAGAGTCTCCCTGATATTTTTTAAAAATCTATT
TCCTTAGAACATTATACTTTAAAATCTGATATGAAGTTCACACACTAGGCATCACATCTATTTATTTAATTTATTTATTTAGCATAA
ACAGAATGCCTACTGTGAGGTACAACCAATTTAAACTTATCCACGACTGGCTTACAAGTAAATGAGACTCAGATTATGGTTATTTTA
TTTGGCTTTGACAATTACTGGGGGGTCACCCCTAATCGACTCTTCTAACTGCTGCCCTGGCTACCTCCCCAGCCGTGTACCCCAGAT
ACTTGCTGTTTCTCCTGGCCATGTGCTTTCCATTCATCTCCCCTCATGGGTGCTTCTACTCTTTTCTTCCTCCTTCTCCACTGGTCT
CTCCTCCAACCAGGTCACAGATACCCCAAGAACCTCTAATCCCTTCAGTAACGGAGCTGGTGACCATCAGAAGTCTGTCTGCTGCTC
AGCTCAGAATAGAGCAGATCACCGCTTACTCTGTGCTCCCCAGTAGCTCCACTGGCTTTCAGTAAGACATGGACTCCCTTCCCCTCA
CCTCCACACCATACCATTTCTGTCTCTCATTAGAAAAACAAACAGGCTTCTAGGGAATAATAATAAATAAGATAAAAAGAAAACTAA
CACACCAGAATTGGACAAAATAACCACAAGGAAAAGAGCCCAAGAGAGACATAAGAAACAGAGACCCACTCATGGGCACGTTCAGGA
AGCCCATGGGAACACTAGGCTGGAAAACTGGAAGCCATCATATATGTGCAAAGGATCTGTGGGGTAGAAAGAAGAAAAATTATAAAA
AATACAATAAATCTTCTTTGAAGGTCTGATAGAACTCTGCAGTAAACCCGTCTGGTCCTGGGCTTTTTTTGCTTGGGAGACTATTAT
TGGCTGCTTCTATTTCTTTAGGGGATATAGGACTGTTTAGATCATTAACCTGATCTTGATTTAACTTTGGTACCTGGTACCTGTCTA
GAAACTTGTCCATTTCATCCAGGTTCTCCAGTTTTGTTGAGTATAGCCTTTTGTAGAAGGATCTGATGGTGTTTTGGATTTCTTCAG
GATCTGTTGTTATGTCTCCCTTTTCATTTCTGATTTTGTTAATTAGGATGCTGTCCCTGTGCCCTCTAGTGAGTCTGGCTAAGGGAG
TATTCTGTTGGTATAGCTTCAAGTTTGTCTGTGGTGTTATTCCAAGCTTTCTTTCCTTATTGGTTCTTGATTTGCTCTATAATTATT
GGAAGTGATGTCTTGAAATTGTCAGTAATGACAGTTTCATGATCATATTTCTGTCCACTCTCCCACTCTTTGATGCACTTTAGAGGC
TGTCTCTAACATTTAGGTGTGCAGATGTTTGCAGTAGCTATAGTTTCCTGGCATGCTAACCCTGCTATATCTTGTCTTTCCTTTTGA
ATGGGATCTCATTCTGTAGCTCAAGCTATTAACTCATTCTACAGCCCAGGCTAGCAAGAATTTTTCCCCTTAGTTTCCCAAGAGCAG
AGATTACAGGGAGGAGCCACCGGCCTAACTCTTACTTTTTATCTATAACAAGTTTGCCTGAAGCTCTGTTTTGTCTGGTTCTAGTTT
AACTTCACCCTCACTTTGGGCAACTGTTTTATTGGATTAGAAGTCTTCCTCATCTCACCAACTGTAGCACTTGGGATGTGTTATTTC
GTAGCCCTTGGCCTTACTGAGAAACCTCTTCATTTTATTGAAAAGCTCTGTGTGTCCTGATTCACTAGAGTCCTTCCTGCTATTTTT
CAGGAAGCTCCGTCTTTCAGATGTTTGATTGTGCTGCATTTGGGATCATGTGTCTGTGCCTTCTGCAGTTCACTGAGCTTCATGGAT
GGGTCTGCGAGTGTCTTCTTCACATTTGGGGAGTTTTAGGCATTCTGTCTTCAAATCATCTTTTCTCTTCATTCTTCCTCCCCTATG
GGAATTTTACAGTACAGGTATGCATTTGCTTGATGGTGCCTTTTCAGGCCTTCATTTTTGTTGTCGGTAGTTCATTTTTCTTTTTTT
TTCCATTTTATTAGGTATTTAGCTCTTTTACATTTCCAATGCTATGCCAAAAGTCCCCCATACCCACCCACCCCCACTCCCCTACCC
ACCCACTTCCCCTTTTTGGCCCTGGCGTTCCCCTGTACTGGGGCATATAAAGTTTGCAAGTCCAATGGGCCTCTCTTTCCAGTGATG
GCCGACTAGGCCATCTTTTGATACATATGCAGCTAGAGTCAAGAGCTCCGGGGTACTGGTTAGTTCATAATGTTGTTCCACCTATAG
GGTTGCAGATCCCTTCAGCTCCTTGGGTACTTTCTCTAGCTCCTCCATTGGGAGCCCGGTAGTTCATTTTTCAAGTTCTCTGGTTTT
CCACCAGCTCAAATATTTTTTCAAGTTCTCTCAAATGTTTATTTTAACTAATATTCTTTCAACTCCAGAAATCCTACTTAAAAAAAA
AAAAACAACAAAATTTTTATGTCTGTATTCTCTATTTGGCATGACTTATTTTTATGCTTTCCCTAGTCTTCTAGAGAAGGCTTCCTT
TAATCCTGGATCTATTTAAAATAGATCATTTAGGATCTTTAGCAAGTTCAACATCAGGGCTTTCTTAGGAACAGTTTTTGTATCTAC
AATGTACGTTAGTTACTTATTTAGCTGTATGTCACACAGACATTTATTGAAGAGTGGACATTTTAAATGATGTGATATGGTAATCTA
GGCATCCTGTTGCTTCCTTCCTTAGAGCATATTGCTTCTACTTCTTGCCTAGTGGCTTTTATGGACTAAGTCTGAAAAGTCAGTATT
CATTGTTATGTTCAGCCACTGAAGTGCCTGATCACCTTGGTGCTCAGTTAATGATGGGACAGAGATTATGTTAGGTGCATTGGATCA
GAAAGTCTCAGCCTTCACCAACATTGGAGCATGTTGGAGCACATTGGAGCATGCCACTAGCAGTGTGGTGGAGAGTTTACAGATTTG
GCTTTCCCTTCATTTCATACGTGTGTTGAGCCTCAACATCAGCTGGGGTGAGGGGTTAGAGCCTTCTCTGACCCTTCCTGCACATGC
TGGCAGCTCGGTTAAATGGATGATGCTCCAAGGACTAACACACCAGGTTTGCCGAGTCCCCTGTGGGCAGCTTATTCCCCCAGCCTT
TCCTTTAAATGTGTGACCAGTTTCTCATTTGCTTCAGCTATCATTCCTGCCTTAAGCTCATCAGTATCCATTGCTGACAGGTCTGAA
GATTACTTATTTTAAAAACATTTTTATTTATTCTTGGAAAATGTTACATGTATCATGTATGCAATGTATCTTAATCATATCCAGCTC
CCACTCCCCATTCCAGTTTCCCCATCCCAGACCCTTCCCAATGCATCTCCCAAACAATTTCATGTCTTCTTTTTAAATGTTTATTTT
TAACCCCTGACTCCAAGTAGAGCTGCATATATGTGTGCATAGTATAAGGTCATCCATTGGGCATGGTGTTCCAGTGGCCACACCTCT
CCTAAGTACAGTGACTCTCCCTCCCTGCAGCAACCATCAACTGCAATACTTCCAAAGTGAGGGCTGGGCCATCGTAGCCCTGCCCCT
ACCCATGGTGGAATTTTAACTTAGTTCATGCGTACAACATCCATGTCTTGTTCAAAAGACAGCATGCACGCGCGCGCGCGCGCGCAC
ACACACACACACACACACACACACACACACCACACACACACACACACACACACACACACACACACACACACACACACACACACACAC
ACACATTTAGTCTTTCTTCAGTGATGTACCCTGTTCCCTAAGCCTTTGGGAGATATTTTATTCACAACAGGGACTTGCTATATAGAA
CTTGGGTTGGCCCGGTGCTATATCCTAAGCTGACCTCAAACTTACAGCAATCCTCCTGCCTCAAACTCCCAGGTGCTGAGATCTATG
TGTGAGTACTATGCCCACCAGTTTTGCATGGGTCTTTTCAGTAGAAGTCCGTGGAACATTAATAGTGATAAGAACTGACTCAGATTA
AATAGACACCAGCAGGTTACACAATGACGGTTCTCTGGCAATGGCTCTGGGAGTTACTGTCTTCTCTGCCCACTGCCACACTGTGCT
GGTGAGTCTGTCCTGTCCCTTCTATCCTCTTTCACACTGAGCTGGAAGGTCAGTGGGGTAAGGCAAATTAAAACAGCACAGGTTGGT
GCTTATTGACATCCAGGAGGGTTTTGGTCAGTTCTGATGCTCTGACAAAGTTGATAAATTGGTGTTTGCCACTATCTACCAACATTT
TCATGCTTTACTGGAGGAATAGCAGGGTCTCACTCCATCTCTCCCATTGACGTCATACTCGTCTTAAAACACTAACAGTGGCACTGT
TTATTAATAGCCGTAATATCGACAAGAGGATTTGTAAAGCCCTGTCTAGCCATGCTGTTCTCACCTTGTTTGTACACACTGCTGACA
TGCGCACTTCTGCTTCAGAGGCAACCATGGCCTGGCTTTACTTGTGTGTACTTTTGTTACTTTATTGTATTGTGTCTTTTGCCCTTT
ATAATGAAGCTTAAAGGATCAATATTGATCATGTTCTCTGCTTTAAGAATTCATGAATTCATAGATATCTGAGTTTTTAACTTTGCT
AACATAACAAACATTTCCTCTTGCACTCTAAGAATGTAATTCATGCTTAAAAATTCCAAATTTTTATATTATTGTTTACTATTAAAG
ATGGTACTTTATACCTCAAGCCATTTCCAGATGGGTCATATAAATTATATCCTATCAACAAAATATCTATTTCTTTGCAGCCTAATA
TCAATGAAATGTACTTAAAATTATTAGTGAAGTTTAACACCTTTTATATATTCTTAGTTTCATGCAGAATTCCTAATTTTTCACTGT
TGTCTTATAAGGAGCTGCATCTACCTCGTGAAGAGTTGTTGATTAGTTTTTCTATGTTGTAGAAAGTCACCATGAAGATTATCATTC
AGGAATATGTGATGTTGAAGGCACTTGTAGACTATTTTCTTTAACATTAACTTGTGTCTTCATCTTTGTCACAAGTATTAAAGTACT
GAGTGACATCTTTTGGTTTTGGCAATAAAGCTAGAAATCTTAATGTTTCTTAGATGCTGGTAACATCATGGCTCTCACATATTCACT
GAAAACTATCTTTTTTAATATAATTTTTTCTTAGGTATTTTCTTATTTTACATTTCAAATGCTATCCCCAAAGTCCCCCATACCCCC
CATTCCCCTACCCACCCATCCCACTTTTTGGCCCTGGTGTTCCCCTGAACTGAGGCATATAAAGTTTGCAAGACCAAGGGGCCTCTC
TTCATTGGATGGCTGACTAGGCCATCTTCTGATACATATGCAGCTAGAGACACGAGCTCTGGGGGGGGGGGGGGTACTGGTTAGTTC
ATAATGTTGTTCTACCTATAGGGTTGCAGATCCCTTTAGCTCCTTGGGTACTTTCTCTAGCTCCTCCATTGGAGGCCCTGTGATCTA
TCCAATAGATGACTGTGAGCATCCACTTCTGTGTTTGCCAGGCACCGGCATAGCCTCAAAAGAGACAGCTATATCAGGGTCCTTTCA
GCAAAATCTTGCTGGCGTATGCAATGGTGTCTGTGTTTAAAGGCTGATTATGGGATGGATCCCCGGGTGTGGCAGTCTCTAGATGGT
CCATCCTTTCATCTCAGCTCCAAACTTTGTCTCTATAACTCCTTCCAAAAACTATCTTTTAGCATTAGCTACAGTTTAAAGCACCAC
AGCCTTTTCTCGGCATATTGGATTGTATGATGCTTTGTTTATTGAGAGAAATGTGTTCTGAATGCTACATAAAGGATGCACAGACAA
GATTTTAGCATTGCCACCATAAAGGGCTCAAGGTTTCTTATTCTGCGATGTACAAATAAAAAAATGGAAATAGGCATCTTTCTACAG
ATCTACAGCATTGTGCTTTCCTCAGAACCTGATGTGATGGTAATTCTTTTTTCTTTTTCTCAGTTTAAGACCAACCCTCCAGCTGTG
ACTTTCGAGCTAACGGGAGAGACAGATGGTATATTTAAGATAGAAAAGGATGGACTTCTGTATCACACAAGAGCCCTGGACAGAGAA
ACAAGAGCGGTTCATCATCTGCAGGTGAGCTGAGACAGTGGGATGACACACAGTGGGATGACACTCAGTGGGATGACATGCAGTGGG
ACAACACACAGTGGGATGACACACAGGCTTCTGTAGCACGCTCAGTTCCCGAATCCCCTGGGACCATTATCCCTTGCTTCTTGTTCA
GTTCTCCTGGCATTAGTACAAAACTGCAGTCCTCATATGACCTCCCTCCCTCCCTCCNNNNNNNNNNNNNNNNNNNNCCTCCCTCCC
TCCCTCCCTCCCTCCCTCCTCCTCTTTCCCCCTTTCCTTTTCCCCTCATTCTCCTCGCCTCTTTGAAACCGACTACAGAGTGATTTT
TGACCATAGTAATCCAATTTACATGATCAAAGTCCTCAGATGGCCATACTCATTTGGGTACTGGGGCTGAAGCCCAGCACTGTGCTC
CTGGTGTGCCCTGGTGCTTGGTATGTGTGCCTCACACCCACTATGAAGGCAGCATCAGTGCTGCGCAGAGCCACTGCGGCCCCAGAA
TGCAGAAGGAACTCATGGTCCTACTGGAAATGCTCTTCCTTTCCGTTTCCCCATAGCTTCCTAGTAGGTTTCTGAATAGCTAGAAAG
GCAGAGCGCTAACTGAAAAAGAGCAAGCTGAAACCATCTTCACTTTCTGCCATGGGCTCTGTAGACTGACCACTGTGCCAGCTCTGA
GGAGTCATAGTACACCATCACCTGAGTAATGTTCTGCATCCAGTACCCTGAACATCTAGAAAGCTACTGCCATCATCAGGGTAGCTT
AACTGATTGGACCCTTATTTTAGCAGTATATAATTTGGGCTTTTGTGGGTTCTTCATCTCCTGCAGTCATCAGGTCATTGGTCTTTT
GAATTTTTAAGTTCCTATAAGTGACTTTGGGGCATGTATCTTAGTCCAGAGTCACAAGGTAGCTTTCTTTTCTTCAACAACAGCTTG
CAGCCTTGGATTCTCATGGAGCTATAGTGGACGGTCCAGTCCCCATCACCATAGAAGTCAAGGACATCAATGACAACCGACCCACGT
TTCTCCAGTCAAAATATGAAGGCTCAGTGAGGCAGAACTCTCGCCCAGGTAGAACAGTCAGTCTGAGTATGTCTGTGGGGTGATGAA
AGAGTGGAATTGTGTGGCTGACAGTTGTCACTATAGCTCCACAAGACTGACTATCTAATGTAAATTTCATAGCGATCTGAATGCTGT
TTTATTGTGCCAATTGTTTTGGACCAAAGTCTCTCATCCTTGGCCCTGTTTAGTCTTGGGTAATGTAGGATGTTTGGAAATATCTCA
GACCTCTATCTATGAGATGCCAGGGACACTCCTGTCTCTAAACTGTGACAATAAAAAATGTCTCTAGGAGATCTAGAGAGAAGGCCT
AGCAAGAGCCTTTCTGCTCTTCAGAGGATCTGAGTTCAGTTCTGAGCACTCTTGCTGAGTATCTCATAAATTGCCTTTGACTTTGGC
TTTAAGGAATTCATTGGTTTTGTTTTTGTTTTTTTTTTTTTTTTGTTGTTGTTGTTTTGCCTCTGTGGACACCCACATTTGTGTGCC
CATACTCCTCCCCTCAATTAAAAATGGAGTAAATCATTGAAAGAGTCTCTAGATCTTGAGAAATGTACAGTGTAAGTCAAAACTATG
GTTCTCTAGTGAACTTTCACTAAATACCACACAGCTTGAGGACATCGAAGGACTGTCTGGAAAAATGAGCTTCCTCTCCAATATCCT
TGTGGGATACTTAGAGTTACTGTTGGCTCCTTTCCTTATTGCTGTTCCCCAGCTGTGGCCTGTGCCTTCTCTATGTTGACCTACTCT
CTATTTCTAGATCCTTGATCTTTTCATCAACTTATAACAAGGAATCATTTGTGTTATTCAACCCCTTTCTTTTCTCAGTCTCCAGTA
GGCAAGTAACCATTCAGGGATGGCTGCACTGTCCCTGTCCCTTGTCCCTAGTGGCATCCTTGTCAGTGTTTAGTATCTGCTTCTCCT
GGGTGAGTGGGAAGAAGTGTGGTTTGTTCCCTCTGCAAGCTTTCATGGTATAACTATTTACCATGGCCTATTTCAAATTACTAGTGA
TATTAATACATGACTTTATAGACTTTGGGTCTGGTTGGTTTTAGCATATGGTGTTTTTATTTGTATGCTTTTCTTGTAATCAAATTT
ATACAAACTTTAGAAAAATAACTGACTGAAAAATTTCTGGAAATTCAAAATTGTGCTCTCCCAAGGTGAGTCCAGTGTAGCACTGGC
TAACATCGTAGAAAGTAAAACACTGACTTAGTGAATAATTTAAAAGAGCACAGAGTGTTTAGTGAAAAGTATATTTTTCAAGACAAA
AATAGTTTAATGAGAAAAATGACATCATTTGAATCTTTGCAAGTTTCTTAAATGTCTGGATGAATAAAAACAATTGGGATAAAGGTC
TGTGTCTGGATCCATGATGTTCCCACATGCTGTTTTTGTTGAAGTAGTATGTGACGAAAATTAGTCTCACACAGATCTGTAGCTTAG
GAAATGAGGAATATTTTAACAGCATCTTCAGATATTATGGACATTCTTTCATATACAACCAAAGCTTGAAAAATGCATTTTTCTTGC
AGATGGTAGCAATATAATAATGGTATATTTTGTACTATTTTACTTTAAAATCCACTAAATTATCTTGTTCTTAAATGGACCTTTTCT
AGATGTGATTTCAAACACCATAACTTTTTGAAATATTCCTGAGGACTCCTATAGGATACAAAATAAAGATTAATTTTTCCAGTTAGG
AACTGAGGCAATTTCCTCCACTCTCTTTGCATCTCCTGAGGCAGACATGGATACAGTTGCTTTAAGTTTCAACTCTCTTCTGCGTAT
GCTTTGTCACCAGGAAAGCCTTTCATGTATGTCAATGCTACAGATCTGGATGATCCGGCTACTCCCAATGGCCAGCTTTTTTATCAA
ATTGTCATCCAACTTCCCCAAATCAACGATGTTATGTACTTTCAAATCGACAGCAACAGGGGCAATAAATCGCTTACCCCAGAAGGT
AAGTCAAGGCAAGCCCTCACAAACCTCAGCCAGAGGAAGGTCTGTCACCTGTGGGCACATGTCTTTTCTCCTTCTCCTCCCCAACTT
CTACAGGATCCCAGGAATTGGATCCAGTGAAGAATCCTTCCTACAACCTGGTGGTCTCGGTGAAGGACATGGGGGCCCAGAGTGAGA
ATTCCTTCAGTGATACTACATATGTAGATATTTCTATTAGAGAGAATATCTGGAAAGCACCAGAACCCGTGGAGATTAGAGAAAACT
CGACTGATCCTCACCCCATCAAAATCACTCAGGTAGTGTCCAAGTGACATTTATTGCTAGTTCGGATCCATACCTTGATATGTAGAG
ACATATTTTGTGGTCTTCACTAATTGTTTTACTAGATAGAGTTTTGCATCATATAATTACAAAGCAAACTAAGTAATCTCCAAAGAT
TATCATCACAAGCAGAAACTTGGCCCATGTGCACAAACGCGCGCACACACACACACACACACACACACACACACAACACACACACTT
TATTGTGGTGTTTTATTGTTTTCATTCAGCAGTGTGAAATATAGCTGCTTTATAGTAGGTTGAATGGCATGTGGTATACCATAAATA
TATGTTGTTAGTAATAATATCAGAGAAACCAGGCCTTGTCTATGTATCAGGCCTTACTTTTGAATATTTTTTGTTGTTGTTTGGAGG
ATAACTAGGGGTCAGTAGTTATTTTTATTTCTGAGATCTTTCAGCCTTTAATATTTGAGGGAGTACCCCTCATCATGTGTCTCAACT
AAATGAATGAATGAATGAGTCTAAGTCAGTTGTTCTCAACCTTTGGGGGTTGAATGACCCTTTCATAGGGGTCACATTGGATATCCT
GCTTATCAGATATTTACATTGCGATCCATAGCAGTAACAAAATTACAGCTATTTAAGTAGCAATAAGAATTTTTTTATGGTTGGGGT
TACCAAAACATGAGGAATTGTATTAAAGGGTCTCAGCATTAGGAAGGTTTAGAACTACCTCTTTAAGGAATCCTTTATTGCTCTGGA
TTCATCTTAATTTTGGATTCTAGAAGCTCATCATACATCATATGAAGTGCCTTTTTTCTTGATAAATTGATGTTATACCCAGTCAGA
GTTCTAGAAGAAAACAACCTGTGGGCTCTTTGTGAGTTTATAGATTGCTTTAAGGAAGAAAGAATTCCCTTGTCTGCTTTTAAAAGG
TTAGTTTAGGTTGAGAATTTTAAATATGTTACTCAGTTGCCTTAAGAAACTGCTCATGTGCAGACAGAGTTGTGAGAAAATTCCAAG
TGAGAATATAGAAGCTGTTGATCACCTTTTCAGTCTGTTAATTCTTGTGTTTTTTCCCACCACTCGCTTTGGCCGTGCAGGTGCAGT
GGAATGACCCAGGGGCCCAGTATTCCTTAGTCAACAAGGAGAAGCTGTCGCCGTTCCCATTCTCGATCGACCAAGAAGGAATATTTT
ATGTGACTCAGGCCTTGGACCGGGAGGAAAAGAACTCAGTGAGTGAGACGGGATGTTCCGTGGGCCACAAGACAAATCCTATGTCTA
CTTACAATTGAGGACAAATTAATTGTAAATTATTAAAAAAAAATGGAACAAAACTGTGTTCTAGATCCTGGATGTTTGTGCATTCTC
AAAGACACAGTGATTCGATAATTTCCCCACAAGTGTAGCCAACATACTTAGAATGGAGAATTGAGGGGGGAGTAAGTATGTCTGTGG
GATGATGAAATTCACTCCCCCCCCTTCATTTCTCAATTCTAAACATGTTGACACTAAGGTGAGAGATGGTTAGCAAAACAGTTATTT
TCCTCCACCTGTTAAAGTGAAGGCATCTTCTTACCTTTGACCTTCACATTGCAGCATGTTTTCTTTGCAACTGCCAAGGATGAGAAT
GGAAAACCACTTGCATATCCACTGGAAATTTATGTGAAAGTTATTGACATTAATGACAACCCACCGACATGTCTGTCTCCAGTGACT
GTATTTGAAGTCCAGGAGAATGAACCATTGGGTAAGAAATGAGAGCTGTTTGATGTCATGAACCTGCCTTCCTCAGCTGCTCCTGTT
ATCCCTTTCTAGCCTCTGGGGGGTCAGGTAGACCTATTCTGCAGTCCATTGTCCTTGTCACAGACCTTGAAGGTATCATGAAAGTCA
TATAGGTACAACACTTTGAAATGAACACTGTCTCTGGTCCTCTTAAGTTTGATGCTTTTATAACTGCCACCACCACCACTGTTTCAA
TGACAGTCACTGCAGAGCAGCATCTTGTGTACAAGTCCAGAAGGGTCCTTGTTGCAAGATTTTCCCTTCAGATATCTTATCATGCTT
CTAAACAAAATACTTTCAAGAGATAAAACCTCATCCAGCATGCTAAAGAACTTGATCCTTTACATCCACAGCCAGATATCCCACAAT
AAGTAGACAATACCAAGTTCAATGTAACCCTGTGGTTCCCATTTGCGGAAGCCAAAGATAACAGAAACCTGGCAAGTGAAGTTTTAA
GCCTCAAGCATGCTTGTCCTTTTGATCTTTCTATGTTTGAGAAGAATGTATCTCCTGATAGCTTTACATTTGTATAGGGACGACACT
GTAAGAATGTTAATGACTTGGGGGGGGGTGTTGTCTCTCTCCTGGATTTGAAGCTTTTGTCTATTTCCATCTTGGAGGTGAGCCCAT
TGAGTAGGCTGATGTGTCTTTTGTGTTTGGGTTATGTACTTTTTAAGCTTGTGAGATCAGCATCTTTGTGGTCCCCATGGGTATGGC
CTACCCATCTAGGGATGGACATTTTCTTTGATCCTTCTGCTCTGGCTGCAACTCAGCTTTGACACAGCAACTGCTTTCCTGGTGGCT
TGGTGGGTTTTCCTCCCATGTAATCATTCTTTGCCTCCTGGTGTTAGGTGTCAGTCACACAGAGGTTGAATATTCTCTGTGTGTCCT
GCACTGGTGTTCTTCATTTTATTTATCTAAATTCCTGCNNNNNNNNNNNNNNNNNNNNNNNNNNNNNNNNNTATTGTTGTTCCACCT
ATAGGGTTGCAGATCCCTTTAGCTCCTTGGGTACTTTCTCTAGCTCCTCCATTGGGGGCCCTGTGTTCCATCCAATAGCTGACTGTG
AGCATCCACTTCTGTGTTTGCTAGGCCCCTGCATAGTCTCACAAGAGACAGCTATATCTGGGTCATTTCAGCAAAATCTTGCTAGTG
TATGCAATGGTGTCAGCGTTTGGAGGCTGATTATGGGATGGATCCCCGGGTATGGCAGTCTCTAGATGGCCCATCCTTTTGTCTCAG
CTCCAAACTTTGTCTCTGTAACTCCTTCCATGGGTGTTTTTTTCCAATTCTAAGAAGGGGCAAAGTGTCCACACTTTGATCTTCGTT
CTTCTTGAGTTTCATGTGTTTTGGAAATTGTAACTTATATCTTGAGTATTCTAAGTTTCTAGGCTAATATCCACTTACCAGTGAGTA
CATATCATTTGAGTTCTTTTATGATTGGGTTACCTCACTCAGGATAATGCCCTCCAGGTCCAACCATTTGCCTAGGAATTTCATAAA
TTCATTCTTTTTAATAGCTGAGTAGTACTCCATTGTGTAAATGTACCACATTTTTTTGTATCCATTTCTCTGTTGAGGGGCATCTGG
TTTCTTTCCAGCTTCTGGCTATTATAAATAAGGCTGCTTGAACATAGTGGAGCATGTGTCCTTCTAACCAGTTGGAACATCTTCTGG
ATATATGCCCAAGAGAGGTATTGCAGGATCCTCCAGTAGTACCATGTCCAATTTTCTGAGGAACCGCCAGACTGATTTCCAGAGTGG
TTGTACGAGCTTGCAATCCCACCAACAATGGATGAGTGTTCCTCTTTCTCCACATCCTCACCAGCATCTGCTGTCACCTGAATTTTT
GATCTTAGACATTCTGACTGGTGTGAGATGGAATCTCAGGGTTGTTTTGATTTGCATTTCCCTGATGATTAAGGATGCTGAACATTT
CTCAGGTGTTTCTCAGCCATTCAGTATTCCTCAGGTGAGAATTCTTTGTTTAGCTCTGAGCCCCATTTTTTAATGGGGTTATTTGAT
TTTCTGGAGTCTACCTTCTTTATATATATTGGATATTAGTCCTCTCTCTGATTTAGGATAGGTAAAAATCCTTTCCCAACCTGTTGG
TGCCTTTTTGTCTTATTGACAGTGTCTTTTGCCTTACAGAAGTTTTGCAGTTTCATGAGGTCCCATTTGTCAATTCTCCATCTTAAT
AGCACAAGCCATTGCTGTTCCATTCAGGAATTTTTCCCCTGTGCCAATATCTTCGAGGCTTTTCCCCACTTTCTCCTCTATAAGTTT
CAGTGTCTCTGGTTTTACGTGGAGCTCCTTGATCCACTTAGATTTGATCTTAGTACAAGGAGATAGGAATGGATAGATTCGCATTCT
TCTACATGATAACCACCAGTTGTGCCAGCACCATTTGTTGAAAATGCTGTTTTTTTTTCTACTGGATGATTTTAGCTCCCTTGTCAA
ACATCAAGTGACCATAGGTGTGAGGGTTCATTTCTGGGTCTTCAATTCTATTCCATTGGTCTACTTGTTGGTCGCTATACCAGTACC
ATGCAGTTTTAATCACAATTGCTCTGTAGTAAAGCTTTAGGTCAGGCATGGTGATTCCACCAGAGGTTCTTTTATCCTTGAGAAGAG
TTTTTGCTATCCTCGGTTTTTTGTTATTCCAGATGAATTTGCAAATTGCTCTTTCTAATTCGTTGAAGAATTGAGTTGGAATTTTAA
TGGGGATTGCATTGAATCTGTAGATTGCTTTTGGCAAGATAGCCATTTTTACAATGTTGGTCCTGCCAATCCATGAGCATGGGAGAT
CTTTCCATCTTCTGAGATCTTCTTTAATTTCTTTCTTCAGGGACTTGAAGTTTTTATCATACAGATCTTTCACTTCCTTAGTTAGAG
TCATGCCAAGATATTTTATATTATTTGTGACTATTGAGAAGGGTGTTGTTTCCCTAGTTTCTTTCTCAGCCTGTTTATTCTTTGTGT
AGAGAAAGGCCATTGACTTGTTTGAGTTAATTTTATATCCTGCTACTTCACCGAAGCTGTTTATCAGGTTTAGGAGTTCTCTGGTGG
AATTTTTAGGGTCACATATATACTATCATATCATCTGCAAAAAGATATATTTTGACTTCTTCTTTTCCAATTTGTATCCCCTTGATC
TCCTTTTGTTGTCGAATTGCTCTGGCTAGGACTTCAAGTACAATGTTGAATAGGTAGGGAGAAAGTGGGCAGCCTTGTCTAGTCTCT
GATTTTAGCAGGATTGCTTCCAACTTCTCACCATTTACTTTGATGTTGGCTACTGTTTTGCTGTATATTGCTTTTATTATGTTTAGG
TATGGGCCTTGAATTCCTGATCTTTCCAAGACTTTTATCATGAATGGGTGTTGGATTTTGTCAAATGCTTTCTCCCCATCTAAAGAG
ATGATCATGTGGTTTTTGTCTTTGAGTTTGTTTATATACTGGATTACGTTGATGGATTTCTGTATATTAAACCATCCCTGCATCCCT
GGGATAAGACCTACTTGGTCAGGATGGATGATTGTTTTAATGTGTTCTTGGATTCAGTTAGTGAGAATTTTATTGAGTATTTTTGCA
TCGATTTTCATAAGGGAAATTGGTCTGAAGTTCTCTATCTTTGTAGGATCTTTCTGTGGTTTAGGTATCAGAGTAATTGTGGCTTCA
TAGAATGAGTTGGGTAGAGTACCTTCTACTTCTATTTTGTGGAATAGTTTGTGCAGAACTGGAATTAGAACTCTGCACTAAACCCGT
CTGGTCCTGGGCTTTTTTTGGTTGGGAGACTATTAATGACTGCTTCTATTTCTTTAGGGAATATGGGACTGTTTAGATTGTTAACTT
GATCCTGGTTTAACTTTGGTACCTGGTATCTGTCTAGAAAATTGTCCATATTGTCCAAGTTTTCCAGTTTTGTTCAGTATAGCCTTT
TGTAGAAGGATCTGATGGTGTTTTGGATTTCTTCAGGATCTGTTGTTATGTCTCCCTTTTCATTTCTGATTTTGTTAATTAGGATGC
TGTCCCTGTGCCCTCTAGTGAGTCTGGCTAATGGTTTAATCTATCTTGTTGATTTTCTCAAAGAACCAGCTCCTCGTTTGGTTGATT
CTTTGAATACTTCTTGTTTCCACTTGGTTGATTTCACCCCTGAGTTTCATTATTTCCTGGCGTCTACTTCTCTTGGGTGAATTTGCT
TCCTTTTGTTCTAGAGCTTTTAGGTGTGTTGTCAAGCTACTAGTGTGTGCTCTCTCTAGTTTCTTTTTGGAGGCACTCAGAGCAATG
AGTTTCCCTCTTAGAAATGCTTTTATAGTGTCCCATAAGTTTGGGTATGTTGTGGCTTCATTTTCATTAAACTCTGAAAAGTCTTTA
ATTTCTTTCTGTATTCCTTCCTTGACCAAGGTATCATTGAGAAGAGTGTTGTTCATTTTCCACGTGAATGTTGGCTTTCCATTATTT
ATGTTTTTATTGAAGATCAGCCTTAGTTCTTGGTGGTCTGATAGGATGCATGGGATGATTTCAATATTTTTGTATCTGTTGAGGCCT
GTTTTGTGACCAATTATATGGTCAGTTTTGAAGAAGGTACCATGAGGTGCTGAGAAGAAGGTATATCCTTTTGTTTTAGGATAAAAT
GTTCTGTAGATATCTATTAAGTCCATTTGTTTCATCACTTCTGTTAGTTTCACTGTGTCCCTGTTTAGTTTCTGTTTCCATAATCTG
TCCATTGATGAAAGTGGTGTGTTGAAGTCTCCCACTATTATTGTGTGAGGTGCAATATGTGCTTTGAGCTTTACTAAAGTGTCTTTA
ATGAATGTGGCTACCCTTGCATTTGGAGCATAGATATTCAGAATTGAGAGTTCCTCTTGGAGGATTTTACCTTTGATGAGTATGAAG
TTTCCCTCCTTGTCTTTTTTGATAACTTTGGGTTGGAAGTCAATTTTATTCGATATTAGAATGGATACTCAAGTTTGTTTCTTCAGA
CCATTTGTTTGGAAAATTTTTTTCCAGCCTTTCACTCTGAGGTAGTGTCTGTTTTTTTCCCTGAGATGGGTTTCCTGTAAGCAGCAG
AATGTTGGGTCCTGTTTGTGTAGCCAGTCTGTTAGTCTATGTCTTTTTATTGGGGAATTGAGTCCATTGATATTAAGAGATATTAAG
GAAAAGTAATTGTTGCTTCCTATTATTTTTGTTTTTAGAGTTGGCATTCTGTTCTTGTGGCTGTCTTCTTTTAGGTTTGTTGAGGGA
TTACTTTCTTGCTTTTTCTAGGGCGTGATTTCTGTCCTTGTATTGGTTTTTTTCTGTTATTATCCTTTGAAGGGCTGGATTCCTGGA
AAGATAATGTGTGAATTTGGTTCTGTCGTGGAATACTTTGGTTTCTCCATCTATGGTAATTAAGAGTTTAGCCGGGTATAGTAGCCT
GGGCTGGCATTTGTGTTCTCTTAGTGTCTGTATAACATCTGTCCAGGATCTTCTGGCTTTCATAGTCTCTGGTGAAAAGTCTGGTGT
AAATCTGATAGGCCTGCCTTTATATGTTACTTGACCTTTTTCCCTTACTGCTTTTAATATTCTATCTTTATTTAGTGTATTTGTTGT
TCAGATTATTATGTGTCGGGAGGAATTTTTTTCTGGTCCAGTCTATTTGGAGTTCTGTAGGCTTTTTGTATGTTCATGGGCATCTCT
TTCTTTAGATTTGGGAAGTTTTCTTCTATAATTTTGTTGAAGATATTTGTTGGGCCTTTAAGTTGAAAATCTTCATTCTCATCTACT
CCTATTATCTGTAGGTTTGGTCTTCTTATTGTATCCTGGATTTCCTGGATGTTTTGAGTTAGGATCTTTTTGCATTTTGTATTTTCT
TTGATTGTTGTGCCCATGTTCTCTATGGAATCTTCTGCACCTGAGATTCTCTCTTCCATCTCTGTATTCTGTTGCTGATGCTTGCAT
CTATGGTTCCAGATTTCTTTCCTAGGTTTTCTATCTCCAGCGTTGCCTCACTTTTGGTTTTCTTTATTGTGTCTACTTCCCTTTTTA
GGTCTTAGATGGTTTTATTCGATTCCATCACCTGTTTGGTTGTGTTTTCCTGCAATTCTTTAAGGGATTTTTGTGTTTCCTCTTTAA
GGTCTTCTACCTGTTTAGCAGTGTTCTCCTGTATTTCTTTAAGTGAGTTATTAAAGTCCTTCTTGATGTCCTCTACCATCATCATGA
GATATGCTTTTAAATAGGGGACTATCTTTTCAGGTGTGTTGAGGTGCCCAGGATTGGGTGGGTTGGGAGTGCTGTGTTCTGATGATG
GGGAGTGGTCTTGGTTTCTGTTAGTAAGATTCTTATGCTTGCCTTTCGCCATCTGGTAATCTCTGGAGTTAGTTGTTGTAGTTGTCT
CTGGTTAGATCTTGTTCCTCGGGTATTTATGTTAGCCTCTATCTGCAAACCTGGAAGACTAGCTCTCTCCTGAGTTTCATTGTTCAG
AGTACTCTCTGCAGGCAAGCTCTCCTCTTGCAGGGAAGGTGCCCAGATATCTGGTGTTCAAACCTGCTTCCTGGCAGAAGTTGTGAT
CTACTCACAGAGGTCTTACGATCCCGTGGAGGGTCCTATGTGTACCATGCAGGTGTCTGCAGACTACGTGCCCAAGGTACCTCGGTG
CTGGCGTGCACC2GAAGGGATTTGTGCCCCTGGTCAGGCCGGGTTTTTTGCTTCCCCAATTAATGCAGTCTCAGGTCCAGCACGATT
GGATTGGAGTAGCAGCTGTGTTCCACTCACCAGAGGTCTTAAGATCCTGTGGAGGGTCTTGTGGGTAGCCTGCGGGTGTCCACAGAC
TCCGCGCCCAAGCTACCCCAGTGCTGGTGGGGCACTTCATGAATTCTTTAAAGGAAGAAAAATTCTATTCAACTTGTTCCATATGCA
GCTGTGGGAGGTACAGACAATGCATCTCCTTCTTGAGGGCTTGCTGCTTAGCTAGCTATAAAAACATACACAAGTAGAAATGTTAAC
ACATGAAGTACTGGTTCGTTCCTCTTAAGTTTATACTGTCCTCTTTAGGAAAAAGAGGAAAAGAAAAGTTTCCTCAATTATGATACA
TTTAGAAAGAAGCAGCCGCTGTTACATGAGTACAAATTCCAAATGGCTCTGAGCCACATGCCTCATGGGTGGATCTATGTCTATCTA
TCTATCTATCTATCTATCTATCTATCTATCTATCTATAGATCTATAGATCTATAGATCTATAGATATAGAGAGAGACAGAGAGAGGC
AGAGAGACAGAGAGAGAGAGACAGGAGAGACAGACATAGTGATAGAGATAGAGATATTTCTTCCCTTTTCCATCCTTTAAAACCCAA
CTCCTGTAAGTGTCTGTATAAGGCCTTGGCTGCAAACTGATATAAATGAAGAGTGTAATTCAATTCAAATAGTGGCATCTGAATAAA
TCTCAGGTGGGATTCTGACCGCATGAAAGGAAACTCACACTTCCCATTTTATAGATCTGGATGACCACAGATTCTATCAGAACTTGC
TCAAAGTTGGTAGGACACAGAATCACATCCACTGACCTCAGGAGCTCCTGGGCCAAAATACACACAGGCACAGTATACATATGGCCA
TGGCATGTGGTATGTGTGTGTGTGTGTGTGTGTGTGTGTATGTGTGCGTGTGTGACTATTCTTTATGTGTTATCTTTGAGTGGAGTC
AGTCTCTACAGTTGTTCACAAAGCTCTCAGAAGTAGCCCAAGATTGGCTTCTTCTAGTTTAGCTGTGACATTTGTCCAGTGTGTTAG
TGTCTTGGGGCTATAGTAATAAAGCACCAGAAATGTGAACTTCTCAGTTCTGCTGATGGGGAATCTGAAATTATGGTGACAGGATCT
TGTGGGAAAGCCTGTTGCCTCTTCTGACTCTGACTTAAGCTAGCTGCTGACAACACTCTGCAGATCTCTTTCCTCCTCTTCTTCTTT
ACCTCCCTCTGTGTGTGGATGATCTCACCCCAGTAATGTAGTTGCTTATATCTCTGATCACCCTATTTTCAAATAGGGCCCCCCACC
AAGATTAGAGGTGGAAGTAGATTCCTTTGGGTGGGTGAGGAGCAGGACGTACACCATGCTGAACTGTCACTCCCCACCAAATCTGTA
AGTTGATGTCCTGCTCCTTGTGTGGGGTAAATTGTTCTTCCTCCCCAGCTCATAGGATAAAGTTCTAAGTCTTGGTACTTCAGAATA
TGGCTATACAGATGTAGATGTTTTAAAGAGGAAATTGGGTAGGATGAGTGCTGGGTAGGATGTGGGACTGGGTAGGATGAGTGCTGG
GTAGGATGTGGGACTGGGTAGGATGTAGGGCTGGGTAGGATGTGGGCTGGGTAGGATGTGGGCTGGGTAGGATGTGGGTTGGGTAGG
ATGTGGGACTGGGTAGGATGTGGAACTGGGTAGGATGTAGGGCTGGGTAGAATGTAGGGCTGGGCAGGATGTAGGTCTGGGTAGGAT
GTAGGACTAGGTAAGATGTGGAACGGGGTAGGATGTGGGACTGGATAGGATGTGGAACTGGGTACGATGAAGGATTAGGAAGATAGC
TCAGTCAGTACATTTTTTACCTTGCCTCTATAAGGACCTGGGTTAGATCCCCTGATGCATACACACAAAGCCAGGCATGGCAGTACA
TTTGTAATCCCAGTGCTGGGGAGGCAGAGACAGGTAGATTACTAGGGTTCACTGGCCAGACAACTTAAATATACTTAGCAAGATTTT
GTAGAGAGAGAGTGCTAGAGACCCTGTTTAAAAAAAAAAACTAAATAAAACAAAACAAAAACCAAACAACAACAACAAGAACAACAA
AACCAAACAAAAGGAAAAACAACAAGAACAACAACAAAAAGGTGGGTAGCTTCTACGGAATGCCGCTGAGGTTGCCCTCTGACTCCC
ACATTCACATGTATGTATGTGTATACCCACAAACACCCAGCTGGTGGGCCTTAATCTAATCTGACCAGAGTTCCTAGGATAAATTTG
GGTGCTAAGGTATAAAGAGGAGGTTACATAGTGAGGAGGAACAAATAGCCAGGGAGAGACGCTTAGAGGAAAGCCGTTTTGTCAACA
CCTTGATTTCATGTGTCCAGGGTCTAGGTCTCCCAGAAGGCTGATTTTTGTTTAAGTCTCTCAATTAACAATACTGTGTGGCAGGCA
GTCTTGGCTGCCACTATGTTCAATCAAGCATACCATGAAAAAATGCAAGGAGCCAAAGTAAATCTAAATGTCTTTGCTCACCACCCC
AGACAGTTCCCGTGTTTCCTAAGAAGCCACTTGATGAACTGATGTGGTTGTTGTTCTTTCTTCTCCAAAGGTAACAGCATCGGGATC
TTTGAGGCCCATGATATGGACGAAGCTAACAACATCAACAGTATTTTGAAATACAAGCTTGTAGACCAAACACCCAAAGTTCCCTCA
GATGGACTTTTTCTCATTGGTGAATATGAGGGAAAGGTTCAGTTAAGTAAACAGTCCTTGAAGAAGCAAGACAGTCCTCAGTACAAC
TTAAGTATCGAGGTGTCTGACGTAGGTAAGCACTGTACCCGATGTGGTGAGTTTACATGTCTGTTTTCCTTTTACTAAGGATTTGTG
AGGCCCTTCCCATGCCAGTAGCTTTCTCTGCTATCTTTGTGACTGTGAGGAAGCCAGATGTTTTCTCAAGAGCTAGAGGTGCAGATT
CGGAGACATCGCATTGTCTTTGGTCCTCTGAGAACTAGGTGTCAAGAGGAATTCAGTGACCATCTTAAAGAGAATGCCCTTAAGAAA
AAATGGAAAGGAAGCTTCGAAGTCTAGGGGATCCCCAGACAGCAGTTCTGGCTTGATCAAGATGGAAGGAGTTTTGATAACAATGTT
CTAGACCACTGAGCAGTCTAAGAAGAATTCCAAATGGTTATCGGGAAGGTCTGATGCAGGGCATTGTCTCTGTGGATAGGCCTGTCT
TTGCATCACTACTTGCTCAGTAACCATCTGGGGGCAGCAAATAGAAGATGAGGTACAAGTGTACTGTTGGTCACAGCATAACACTGC
AGCTACCAGGTTATCAGTCCAGCTGTTAAAGTCACCAGGCGCAGTCTCACAGCTACTATAATTAAAATTCAGGAAAAATTCTTTCCT
CATCCTTGCTTCCAGACTAAAAGTATCTGTGTATGAAAGCAGTAGAGAAATTAGTCTTTGCTGTTCTTGAAGGCAGAGAGAGTTCTG
CCCTTTCTTGCCACCTTCGTATTGCCCACTAGACAACAATAGGAAACCTTTCCCCTTAATCCTTGTATACTATCTAAGTGGGTTCTG
CTTAACCTCTCCTCTCCTTAGGGCAGAGGAATCCTTAGAATCTTATGGCAACTTCCCATTCTTAAACACATCTATACATCCACAGAC
AAATTCGCTGCTATCTCTAGGGTACTCAGAGTCCTAGGAGGTAACTGGCTCATGAAATGATTCTGTAATTGTTTTCTGAGGAACACA
GTTTACATAGGTGTGGAAAAGAAAGGACCAGTGGTTCACATGTATTTCTTATTTTTTTTCCCCTTTCCCATGGACCCTGGGAGAACT
TGAGAACTTGAGTTAAACAGGTTCCAGTGCAAACTGCCTGTCAGGTTCTCAGGTAGCTAGTGAACTGACACACGATGAAGGTCTGAT
GCTGTTTAGCACTATGTTAAAAGATCCAGTGAGATAAATGTCAATCATGCTATCCATGGCAAAGGGGCAGACTCTCCCTCACATTGG
CCACCCACACATTCAGTATTTAATCATTTGCCTTGCTAAGGTATCAAAGTATTAGGCATGCACAGATGAACTGGACCCAGTGTTTGT
CCCTAGGGAGTACCCCTTTTGTCAAGAAAGATGGACACACATGTTAACAACTTGTAGGGACATACTATCTACTGTCATTGTGGTGCT
CTTCGTAAACACTGTCCCACAACTCATCTGCAGGCCACACTCATCTCGAATCTAGGATCCACTTGCTGGTTCCAATTAGTCCTAGAA
TTGTCAAACCACCCTCTAAGCCTGGACCATTTCTGTGAGCTACCTTAGCCTCCCTTGGAGGTGAATTATGCCACTTTATTCCTTACT
TGGAGAAGAGGAAGCAAGTGACCTTGAACCTCTCTACCATGCTGGGTAGACACATGCAGAGAGGGAAAGGCAGCCTGTGGGGTGTTC
AGCAGAATTGTGCAAAGGGACAGTTTTGCTTTGGGCATCAGAGGTTATATAACATTCACCTGTCAGGTAAGGGAGCTGCTTCAGAGA
AAAGCAGCCATCCTGGTTGTTGAAAAGCCTTCCCAAGGATAGGGCAGAGGGATGCTCAGGAGAGACTCTCAGGGCCCAGTCTGGCTT
CCACTTCCTCTTGTTGACCTGCTTGTGGATGAGGTGGTGGTGGTTGGGGTGGGGGGCAGAGCAGCCTCCATTCACAGGAACTGTAAA
GTTATAGCAGCAGCTACTGTGTCACTTGTCAATGTTTCCTTACCAAATCCTGATGCCAATGCATGATTTCCAGTGAGAATTCTGTGG
TGTGACTTTGGTTTTCAGATTTCAAGACTCTCTGTTATATTCAAGTCAACGTTATTGATATCAATGATCAGATTCCCATCTTTGAAA
CATCAAATGTGAGTAGATATCACTATTTGTTTTATTTGGATCATATAATTGAACCCTTATTTAAATAAAAACACAGACAATGTGCCA
GGTAGCCTGCTCAATGCATCAATAAAAATCAGGCACCATCTTCACGTGAGTCCTCAGGGGCCCTAAGGTGTACTGAGAGTAAGGAGG
GATGTCTATTTATAAACTCTTGCCAACAGCTCATTTATTCTAAATGAGGCTTAAAGGCTACAACCACAATCCTTTTCAGATTGAAAA
TTAAATGGCTGCTGGTGGCTTGTTATAGATTTTGCATACAATGGACAGTAGCTAGAGCTACAAGATCTATGCCAAGCATTTGGGATT
TAAAAAATTAGCATCAGAAATACTGGTGACTTTAACTCATTTAGTATTTAACCAAGTAAGAGGAAAGGAAAGGGGAAAAATATTTCT
GGCTTAAGCCTAAGAGAAATGTGCTCCCTATATTTGCAAGAATTAGCGTTGGACGTAGAGTAAGTGTCAGGGCAGAAAGGACGTCAG
GGAAAATGGCTGTGGTCACAGTGTGGTGTTATCATTGTACCCTTAAAAGGAATGTCTTGGTTTATTTAGCTCCAGGGAAAGAGAAGC
CCACTTGACTAGATTGTTATGTTAGAAGTCAGTTTTGAGGCAGCCCAGAGCCTCCTGATGATGTCCATAAATCAAAACAGAGGCAAG
GCTGTAACAAAGAAGGGAATTAATTTACAGTAGAGCTCAGAACCGTAAGAAGTGTGATGGAACGTATCTTGTCTAACAGCCACTGAG
CTTAAGTAAGCATGAAATAAAGTATTCATTTTTTTCTCAGCCTCTATCCTCTGAGGCATTTTTCACTTCCTAGTATTGTGTGTCCCC
TGCATTTCAGCAGACAGTGGTCAAGTAGATTGAGCAGAAGAATTCTGCAGTGGGGAGTTCAAAAGCTTGGAGATGCAAACCTGGCTG
GAGAGAAAGTGGGCTTGCTTAGTCCATTGCCAGCGTCTTCTCTTGGATGCTGGCTGGTACCATTTTTGAGGTCATGATGTGGGGGAA
GAGTGGGTCATGGGGCTTCAGGGGAGTTAGAATGCTTTGTGGGGTGTGAGGAACACAGGCTTTGGTGGTTTGGGTTCAATCATACAA
GCTGTTAGCCTGGTGTGTGTGTGGGGTTGACTATGCTAGTTCTTGGCTCCTCTGTCAAATGTCACTGACATGTTTGTAGCCCTCTAG
TGTACTTTGCTGCTCATCTTTCATAAGGGCTGTATACCAGCCATTCAGAGGCTCTGCATGCATTATCCCCTAATGACCACAGGTTGC
ATTCTCTTAGTTGACAAGTGACAGAGCCCATGTTTAAAGAAATGAGATAACTGTATCATTCAACTAACAAACAGCAAGTTTAGGGTT
AGTAGCCAACTCTGACTTCAATGTTTTGGGTTCCTAATGTCCATCACTTGGTTGACTCAAGAATTAGAAGTATCTGTCACATCATAA
GGACTTCTCATGTTATGACTACCTATATCATTATTTTTCAAATAATTATAGTTATTAAACATGTGGATATTGGAGGGGAACTCTATT
TTAGAATCCTTGTAGAATGTCTAAGTTCTATAAACTTAGTTTAGTTGGTATAAATTTCGGTTCATGAGTACAAATGATGTCAAACCC
GACTGTCACATGTTCATCAAGAGGAAAACCATGCTGGGTATTAAAGCCAGCTGTGACCTCTGTCCTGAACCCAGAGACGTTTGCCTT
TTGTCCAGTAAACTCAATGCCATGGACCTTCTGCTTCCTTTCTTGTTTCTTCCTCAGTATGGAAGCAAGACTCTGTCTGAAGACACC
GCCATCGGATCCACCATCCTAATCATCCAAGCTACAGATGCTGATGAGCCCTTTACAGGGAGCTCTAAAATCCTGTACAAGATTGTA
CAGGGAGACACGGAGGGAAGACTAGAAGTTGTCACAGATCCCACGACCAATGCAGGATATGTCAAGATCAAAAAGGTAGATATCAGC
CGGGCAGTGGTGGCACACACTTTTAAGCCCAGTGCTTTGGAGGCAGAGGCAGGTGGATCTCTGAGTTCAAGGCCAGCCTGGTCTACA
GAGTGAGTTCTAGGACAGCCAGGGCTACACAGAGAAACCCTGTCTCAAAAAACAAACAAACAAACAAACAAACAAACAAACAGCTAT
CCCACATAATAATTGTTAACAAAATAAATAAAATAGATTATTCTACTCACAATTATGTCAGGACCAATAGGCAGAGGTAACATGTGC
ACATGTACACAGAAGTTATCTGATGTGGATACTGGGAACCAAACTATGATCTCTGCAAATGTTCAACTGCTAAGCCACCTCTCTAGC
TCCCAAGATGAGACTACAGACAGATATTCCTTATGTGAGTAAAATGGCCATGCATAGAATTAATCAGGAAGGTGAGCCCAAGTTTTA
AGACAGAGGAGACAGATGTGAAATGAAGGAAGGTGACAGACTTTTATCCTGATTTTAGTTAGCTTATGAGTCTGAGTGAGGGGCCAG
TGCTTTTCACCACAAGGAATTATGAAGAACTGCAACTCCTTCCTTTACCATGTTTCTAAGCCCAGTTGGCTCTTTTCTCCTTTCTAT
AGACTAGAGAGTTTGGAGATACAGTATTAACTCATAGTCTCGTCTTCCTCCAGGGTCTCTGCAACTTTCCCCCAGCCTTTTCTCATC
CCTCACTAGGTACCTGAGTCTCAAGTTTTCTCTTCTGTAACTGCATAATTTGTGTTAGAGCTCACCATGGGATTACCAGATAACTCA
TTTGGAACCAACACCACCCAATGGGTGTTGAGTTAAAGCACTTCCCATGGGGAGAGTCCAAAGCCTTCCAGGTGAACAAGGAGAGGT
TCAACAGAGAATTGGTTGGAATGCTGCTTGTTGGTTGGGGCTGAAAAAAAAAAAGGCCCAGGAAATGTTTTTAACTAGAGCAATGGA
AAGTTGAAAATAAAGAATAAAAGTGGAGGCCAAGTAGAGTGGTTGCACAGAAAACAAAAAAGAGCCAAGGAAAAAAATAACGCCAGC
CTGGAACCAGCCATTGAATTGCATCAGCTATTTGGATGCGAACTGTTGCCCATAGTCTCATGTGTTTGGACACTTGGTCTCAAGCTG
GTGGTGCTGTTTTGTAAGGCTATAGATCCTCCAGGAGGTGAAGCTTTGCCTGAGAAACTATGGCCAGAGCTAAGAAGTTTAATGGAC
CAGCCTTGCTTCCTATCCATTCATTTGCTTTCTGATAGCAGACACAGTGTGATCAGCTATCCCTTTCTTGCTGAGATGGGCTGTGTC
TCTTCATAAGCTGTGAGCCAGAGTAAACTCTTCCTCCCTTAAGCTGCCTCTTGTCAGGTATTTAGTGACAACAAGAAAAGAGTATGT
ACTGTCCCCTGTACCACATCTGACAAGGGTGCTTAATGGGTTCAGCAGAGTTCAGTGTATTACACCCATAACCTTCTGTGGAAGCCT
GATATTTGGAGAACATATGATCACAGCTTCTTCTTCTTCTATACTTGGCTATTTACATGAAGAAAGAACATGGGAAGGATGGAGGAA
GGGGTGCTTTTCATAATTGGGAGTGGGTAGTCTGCAAAGCATGAGTCTTTGCTTCAGGGCCGGGACATCTCTCTCCTCCCATCACTC
TAGGGACTCAGAGAAGATAGGAAGATGTCTGTCCCAGTAAACAACTGCTGCATGTCATTTTTTTCTTTGTGGTAGCTTATCATGTAG
CATCCTCAGCTTACCTGACCATGTGTGGTGATTGGATCCTTTAGGGAGATGATGGTAGTGGAGAACTATAGCTGTACTTCCACATAA
GTCATATATAAGGTACCATTATAGTGGTGCCAGGCCACTCACTGAACACTTGCCATCCTTGTTTCATGTATTGCTGTATTTACAGTG
TCTAAAATATTGCTTGACACATGGCAGATGAAGATATTGAGTATTGCTTGACTGAATGAATAATTGAAATTTGTTTCATGTCTAATT
TCTAATAATCACATAGCAATAGTATAACCTCTGTATTAGGAACCTGAAGTCTCAGCTCTCCGTTCCCCAGGCACTGTGACTACAGAA
CTTGAGTGTCAAAATTTGGCAGGCTGATCATTGGAAGGTAATCTCAGAACACAGCTGGAGTCCTGGAATTCTGAAGGTCACAAGCTG
TACCAGAGGCTTACACTACTATTGACAATGACACTGTGTTTTTAGCCACACTAAGTACTTAACAGTCTGTCCTCCCATAACACCAAG
CATATCTCTGCAAGAGCATTTTGGTAACGGACATACACTGTGCTGATGGGCAGAGTAGCTGGAGATTTCAGTGGCTCCTTCTGGAGT
GTGGACCTCAGATTAGGCTTTTTGAAGTGCAACAACCAACCCTCAGGGATGACTTTTAGCTCACTGGATGCAGAAAAAGATGTCATC
ATATACATATACATACATACACATACACATACACATACACATATACACATACATCACAAATATATATATCACAGATATATATATATA
TCACAAATATATATATAATATTTATATATTATTTATATAGTATTTGAGTGTGGACTTGGCATCAGTGTATGGTTTATACATGTGTGG
GTGAATTCAGGAGGCCATGGAAGGATGCTGTATATTCTCTTGTATTACAATTTTCTTTCCATCGAAACTCTCCAGGATGAAGCCTAT
GTACCCTCTAACTGATGTACTCTGGGTACTGTTTGTCAGGAAGGAGATAAAAAGTGAGGTGACTATGCCCCAGTCCTTCCTTTCAAC
AGGAGTCTGAGGGTTATACTAGACATAGCTTGAGCATATATAAGACCTCAAAACCCACCCCTACAGTGACACATTTCCTTCAACAAG
GCAATACCTACTTCAACAAGGTCAAGCATTCAAATACATGAGTCTGTGGGGGCCATTCCTATTCAAACCACCACAATATTCTTTTGG
GCTTTGCATTGGTTCTGTTTAGTTTTTGATGTTTTAGGTCCTTTTAAGAATCTGACAACAACAACAACAACAACAACAACAACAACA
ACAACAACAACAACAACACACTACCTGACTATTTGACTGGGACTGGCAAGATGGCTCAGTGGATGAAGGAACTTACTCCATAAGCCT
GACCACCTGAGTTTTATCCACATGGTAAAAGGAGAGAGCTAACCCCCACAACGTAGCCTTTCATCTTCGTACATGTACTATGGGATA
CATGTACCAACATACATATACATACACATACAAATAAATAATGGAATTTTAAAAGTCTACGTGCCTCCATCTTAAAATAACAATAAT
ACACACACACACATGCACACACACACACACGGAATGTTTAGATTTTAGTTCACAGGGGTTCAGAGTCCTATGGGATTCTTGAGTCTT
GAGATGGCAGCCTACACTGCAGGAGCTATCATTTCTTGGTCTCTCGTCTCCTATAGAACAATAAATTCCTGCTTAGAGGGGGATATG
AGGTCTCATAGGGCCAGTTGCTCATAGGATAGATACATCTTTCTGTTCTGTTGCATATAGTTAAGCATATCCTTGAAAAGTTACAGA
ATTTGGAATGAATTGTTTTCTGAACCAACTCTTACCTTCCTTAGGTAGCCTGGTTGTTAAATTCTCCCTCATACAGAGCCAGAAACA
ATGCCTTGTTCTTATTACTGGTCCTGTCTATCCAAGTGGACCAAAAACACATCCTCCTGCCGGCATGGCCTCTGCTCACCTCTCCCA
GAGTCTCATGTTTGGGGTCTTGGGTTCTGAAATCACTGGAAAGTGATCCTCCATACCATGTGAACATCTCTGTAGTACAAATATTTT
CCTAGATGCCATTGTCTGAGGAGAATCGAGATTTTTCTGAGAAGACATGTAGGGTGTACCAGGGTTAAATGAATCTGAGGAGGGCTG
TGTTAGAGTCTCTCTTCCTAGACAGCCCATGTACGTGAAACCACTTAATGCCCTGATGGCACCTGAAATAAAGTTGTGTTATGGATT
TCTTGATATGAAGGTTTCAACTAAGATTTATGAATTTTTTAAAAAATAAATCCTTTAAGGGGGCTATTCACCCCAAGATTAATTTCC
CCTTGTTTTCTCTCCCTCCACAGCCTCTTGATTTTGAAACTCAACCAGTTAGCAGCATTGTGTTCCAAGCGGAAAATCCTGAGCCAC
TGGTGAAAGGCATAGAGTACAATGCTAGTTCTTTTGCCTCCTTCGAGCTGATTGTGACGGATGTGAATGAAGTGCCTGTGTTCCCCC
AGCGAATATTCCAAGCAAACGTCAGCGAGGATGCTGCTGTGGGCAGTAGAGTGGGCAACGTGACTGCCAGGGATCCTGAAGGCCTGA
CTGTGAGGTACAGGGCAGCAGGGCTGGACAGGCCTACTTGCATGCTGCAACCATAACCAGAAGGAGGGCTGGCAAGGCAGAGAAGCA
GCGTGCCCTGACTTCAGGACTGGGGCAATGAAGCAGACAGCAGAAGCAGGGTCTCTGGCCTTGGTTCAGTGTGTCAGACACATGGCT
TCAGGTGCCAATTCTGTCTCCTTGCTGCAAGCTTCATTTGAGATCTGGTGAAGGTAGTAGGTGGGAACCCCACTCCCTGAGATTGGG
TGAGCCATGGCTAGGCCCTAGGTTTGAACTATTGGGGTTCTAGAGAAGCTTCCTGGGGAAAAAAGACTGGAGTTGTAGAGAGAGAAA
CACCCAGAAGTAGAGAAAGCCACCCTAGAGGCCAAAGGGAATGTTTCAGAAGTAGGGATTCTGGGCTTCTCCTCTCTCTGGAGAGCT
AGTCTGGGGCTAGTCTTCCCCCTCATTCCCAGAGGGTTAGTGCTCAAGGTGAGATGCTAAGACAGCATTTCTGTTTTAGCCATTCTC
ATTCCTTCTTTTTCGATGCTACTGTCCCCACACATATTTGCCTCAAAAATGTACAATGATTAATTCATTTACTATATACACATATAC
ACATTTGTATAATACCTAAATGATTTTAAAGATCACATAGTTAGTAAACATAGATAGGACTTGAACTCAGACCTTTTGTTTGTCTTA
TACAATATTCTCCAGAATATAAACAAATAATTCCACATGACTTAGCTGGTACAAGTATTTCCAAGTCTTTAAGTTTCTCTGTATACT
TGAGTGAAGTTGTATCTCCTATCGAGCTGTCCTATAGACAGTTGGGTTCAATTATATGGGCTTTACTGATTTTCTTGCATGAAATAT
ATTATTAGTTTGCTTACTATAAAAAGAGTGTGAAGCAGAGAAACTTTATAAAGAAAACCAGTTTATTCAGTTCATAGCTCTGGACAT
TTATGGGCATAGCACCAGTACAGGCTGGGTCTGTTGAAGGTCAGTGTGATTATTAACACAACAGCAACACAAGCAAAGGGAAAAGAT
CACATTGTAGGCAGGAAGCTGTAGCCACTGCATTCTCACAACCCCTGCAAGGGAGTGTCCCTAAGTGATGCAAGACATCCCACTAGA
CCCTACCTTCAACGGTCCTGTATTCAGCAAATTTACCATCTGAGAAGGAGGCTTCTAACACGAGAACAGTTTTTGTGGGGGAGGGGG
AACACTCAAATCATGTCCATATCATACTGAGGAGTAATGTTCCCACTAGTGAGGATTATATTGAATTATTCAGTGTAGAAACAATGC
TATACATCCAGGTCACAAGTCATGATGGAATTCAGAGCCCATTACTATCTAACCAATGCCTCTGTATTCTCTGGGCGAATGCTATAA
AAATTTGCTGATTGTAATGAAAAGATAAAATAAATCAGTAAAATTACTGGCCAGAGGTATGTGCACGTTAGTATAGAGAAACAGAAG
CATTTAAGGAAATTCCTCTGTTGTCTCAAAAACTGGTATAGAGCATAAAACCAGAGTTACCATCAGATGAGACGGGCAAGGGAGACT
GAGAAAGGGCATTCTGAGAAGAGGCATTCTATAATGTCTGTTTCTGGAAGCACGTGAATGAGGAGTAATCAAGGCAGCCAGGGGCAC
ATGACAGAGTAAGAGCTAACTTTATGGACTCTGACATTGTGGACTGGGAAATTTAACTCTCCAGCTGGGTAAACCTAGAGAGTCGAT
CAGCAGATTCAAATCCAACCCACCTTCACATATTATAGAGCTCATTGCAGGCCACAGATCTAGAAGCCACACCTTTCTGACTACTTC
CCCTGTGGTCCACAGCACTGCCATCTCTGTTGTGGACCCATCCTTCCCCACCACTCCATATCTCATTTGAGTCAACTGTGCACTGCA
GGCAATGAGAGCCTTTAAGATGAAGTAAGCTGACTGTCCAGCTCCTCTTCTGAGCTTGCCCTGGGATTCTTGCTTCTGTCAGGGTTG
GATCCCAAGTACTTAGGAGCTCAATTTATCATTCACACAAAATCATCAATACAGTGCTTGACATATGCAACATGCTCAAAATATACA
TGGGAAAATTAATTTGACTAAATATAATTTTAATCATTCATATTCCTGGACATACAAACTAACAGAATTTTAGAAAATTGTAGGCAG
TCTTTTGAAGTCACACACACACACACACACACACACACAATTCCTTGACTCCTATTAAATATTTATTGAGGTGACATTTCCAGGACT
GGAAATAAGTGGCTTGCAACTATAGACATCTTTACAGACATATGTGAGGCTTTACAAAAAGGCTAAATTTACTGGCTTTTGTCAGAC
TTGTTTCTCTCAAATTTATCTTTCCTACACCCACCAATGAATTCTGTTGAGGACACCATTTCAGCCAGGTCACCTTTCCACTCAGCA
TAGCTCGATGGCTACCTCTGGCTTGTATGATCTGGCTCTCCCAGTCTCTGTGGCCACATCTTTTCCACTTACTGCTGCCCAGACATA
TCCATGTACTGTGCACACACCAGGATGCTCCTGGCCTCTCTGCTGGTTTGTGTATCTTTGTGGCACCCTTCTTGTCTGTGTCAACAT
CTTCAGAAAGGCTTTTCTAAGTCTAAGTACTGGATTATCTGTGTATCTAAAGGCTTCATTCTTCAATTCTTGTCTCACCCCTGTCTG
CTGGAGTTGATGCTTCTTTAGTGAAGTTTTGCCAGGCCACCAGCAGCCTATGTCTCCAACATTACTTGCTCAGGTTTCCTTAGCAGG
GTGGCATACTGACAGCTTTCAGAAGACATATACTTCAATATGAAATCTTGATTGCCAGGAAAGCATAACTTTTCTCCCAGAGCTGGT
CACTTCGCCATACATAATAAAACAAGCATAGGACCATGTTCTAAGGCAGTGGTTCTCAGCCTTCCTAATGCTGCCACTCTTTAATTT
AGTTCCACGCGATGTATGACCCTCAACCATACAATTATTTTCATTGCTACTTCATAACTGTAATTTTGCTACTGATATGAATCATAA
CATAAATATCTGTGTTTTCAGATGGTCTTAGGTAACCCTGTGAAAGAATTCTTTGATCCCCAAAGGGGTCACAACCCATAACTTGAG
AACCACTGTTGTAAAGACAGCTCTTTTTCCAAAGAGAGATGTGTAAATGAGATGTTGATTCTGTGATTCTGCGATTCTGCTCCATAG
GACACCCAAATGATGATTCTATGTCGGTGTATAGAAGCACAAAATGTTTTCATGTCAAGTAACACAGGTTTTTTTTTCTGCTCTCCC
CTAATGAAGTTATTCACTGAAAGGCAATATGAGAGGCTGGCTTAAAATCGACTCTGTCACTGGTGAGATATTTAGTGCTGCTCCGCT
GGATAGGGAAACAGAAAGTGTGTATCGAGTACAAGTGGTGGCCACTGAAGTAGGTGAGCAAAACAAAGCACAACCAGATCACAGATT
ATCAGGGGTGGGGGGACTTCAATAAAAATGAGTGGGAGGGCATGAGAAGGTTAAAAATCGTTGCTGAGTATGACCATAAAGCCATGG
GAAGCAGGCACAAAAGGGAGGGGAGGGAGGGAGTGTGAATATGTGAGTGAGTGTGAGTGTATGCACTTGTGTGAGTGTGTATGTGTA
TGAGTGTGTATGTATCTGAGTGTGAATGTGTGTGTACATGTGTGTGAGTGTGTGCTTGTGTTTGTGTGTATGTGTATAAGTGTGAAT
GTGTATGTGAATATGTGTGTGAGTGTGCAAGTGTGTATGAGAGTATGTATGAGTGTGTGAGTTTGTGTGAGTGTGTGTGTCTATGTG
TGTGAGTGTATATATGAGAGAGAGAGAGAGAGAGAGAGAGAAAGAGAGAGAGGGAGGGAGAGTTTGTGTTTTCCATCCCTCGTGGTT
GTGTTAGAGAACACTTTGAGCCAATCTGTGCTTCAACTAGGCAGAGTTTCCCCAAGGCTGGAGGGTTATCAAGGTTGTTATCTGCTC
TGGTTTTCTGGAGAAACTAGAGCCTCTTCTTGGAATGTTCTGGAGTCCCTAGCTAACCTCCCTCTCTACTTCCTCAAAGGCGATTGG
GGGCTTCTCCTAAACCAGACACCAATGGTCCTCAGAGACCCTTGGTGCTCATGCCTTTTTAAACACTTGTGCATTAATGAAACTGGG
AGTTTCATTTCCTTGGGGGTTCTGATTCAGCAGGTATCTGAACCCGAGTATCTGCCACTTCGATGAGGCAAGATAAACTTTGAAAGG
GAAGACAATTGCACAGATATGCCAGAATGTTTGGACCAGCTTTGCAAAGGCCTTGCATTCTATTTAATTCACTTGTGATACAAGTTG
TCTACTGGCTCTGAACACAGAGTTCCAGGAAAATTATCAGATTAAAATAAAGATAAACTCACAGGAATGATATGGTATCCAGGGATA
AGGCTGGTAATTAAAACACTGAGTGTCTTTAAATGGAATTTTCCATCTTGCAGTAGAAATTCTGTGGATCTTGTGCTGATCAAGGAC
TCTCATTAGCTAGTAATATATCTGCTTGACTTATAAAAAAAAAGATGAGAGCAGAACTAATTACTATCAAACAAATACAGTTTTAGA
CTAAAAAATAGTAAACTTTTAAAATACAAAGCCCCAAATTAGAGGCAGACACAGGGAACAGAGAAAGGAATCATAAAAATGTATGGG
AAAAAAATGGAGAAAACCAAAATGACCATACTTGAGTTCAACTTTAGCAAAGTTGTTTATTATTGGAAGGGATGTACAAATAGATGA
AGCTTTGCTGAGACACGGACAGCTCAGTCTCTGCCGCCTGCTACCCATTCTGCTATCAGGATGTATATATTTTAACTGGTGATTTAA
AAGCTCCCAGCAAGTCCAAGGGATGGATGCTGTGTCTTCTCCTTGATCCCATGACAATCAGCATTTCACTTTGCACAAGTTTCTAAG
TGGTTTTCATGTTTTAGAAGTCACATGGATTATGACTGGGAGCCAAAAACCTGTTCCATTTTCAGTTGTTGGCACCAAAATAATAAT
AATGTTAATAGACAAAACAATTTCAAGGACATCAGTGGACTCATCTTTGATTTAAGAAAAATCAGAGACATAAAAGTTGCTGTAAAG
AAAATGAAGAAAACAAAGCTTCCTGGAATCAAAGCAGGAGCCATAACAGTTTACAGGAACACAAGTAGTGACCAACCATCAAAGCAG
TGTCTGCGTGAGGTCAAGCAGGGACAGTCAGGGTCGATTGTACCACACTGGACGCCTCCTGTGCACTTCTGTTATTGACCATGCTTT
GTGCACAAAGAAACCAAGGCATTGAGAAATTGATTTGCCCAAGAGTGCACAGTTACCATCGGTCATAATGAAGACGACATGGTCTTC
ATCACTATAGCATGACAGGACATTTGTGCAACCATTTGCTGTTTTCTCTTTGTATGTGACATATCCACAATGATCTTCACATAGCCA
TGTGCTTCTCAGCTGAGGAAATGAAGCTTAGAAGACTGAGTTAGGATGTAGAAGGTAGTGGAAAGTGATCAAGGGTGTGGCTCATAG
GGACCTCTCCACTGTGCACTAGCTCTGTGAACTTGGCTAGGTCACACAGCTTTTGAGTGTCATTCTTCTCCTGTGTAAAGTTCAAAG
TTCTTTTGAAGATATACACATACACACACACTCACCTGGTTAGTATTATCATTTAAACAATTAAAACATTTTTTTGTGTTTATTTGT
GTGTGTGTGCCCATCTGAGTGTGCGTGTGTGCATGCATGAGTGTGTGTGTATGTGTGTGTGTGTGTATACATAAGTGCATCAAAGAA
TAATGTATGGGAGTCAGTTCTCCTTCCTCCATGTGGCTTCCAGGGATCGAAGCCATGTTGCCAAGTTTGGTAGCAAGTGTCTTTACT
CACAGAGTCATCTTGTTGGTTCCATTTAAACATTTTAAGCAGTACTGAAATTAAGTCTAATTCTAAAAACTTCCTTCTATCACTTTA
ATAGACCCAACTACTTAAATATTGATATTTTTGAGGAAAGAGAAACATGATTCCACGTGAAGGATGATCATAATACTTAGAATATAA
TTCTCACCCTTCAATTTCTTAGGTGGGTCCTCTCTGAGTTCTACGGCAGATTTCCACCTGGTCCTCACAGATGTGAATGACAACCCC
CCTCGCCTAGCTAAGGACTACACAGGCTTGTTCTTCTGCCATCCCCTCAGTGCCCCTGGGAGCCTCATCTTTGAGGTCACTGACGAT
GACCAGCAGTCACTTCGGAGGCCCAAGTTTACATTCGCCCTTGGCAGAGAAGGCTTACAAAGTGACTGGGAAGTTTCAAAAATCAAT
GGTGAGTTATCAAATGTACAATGAGAAAAATCAATGGTGGATGAAGGAATTACTTTGTGTGTGTATGTGCTAAAAACTTGAAGCATT
TTCACTAAAATCAGGAATAAACCAGGAGTGCCCCACCCTCCCTGACACCTGTTCAGTATAATAACTTGTAACATTAGATAAAAAATA
ACTCTGCAGCCAGTCCCACAGCACCCAGAGGAAGCTCCACTCACAGGCACTCTAACAATCCCAGGATCATAGGACCAGAGGTGAAGA
GGACACAACATCTACCCCAACATTGGAGGTAACTGGGACCAGCCGGACACAGGCATCCAGGAGCTCTGCCCACATGTGACACAGGTT
ACTTCCTCCCTGGGCTGGTGTCCTGAGCAGAATTTTGGTTCAAACTCCCCAGCCAGTTTCACAACACCCAGAGGAAGCTCCACTCCC
AAGGGCTCTGGCTCTCCCAGGATCATAGGATCAGAGGTGCCCTGAGCAGACCTTGGGTGTGAACTCCGGAGCCAGTACTACAACACC
CAGAAGTTTGACTCCCAGGCACTGTAACACCCCCAGGACCACAAGATCCCAGGATCCCAGGATCCTAGGAGCTTGATCACACTAGGA
TCTTAGGGTCCCAGAGGCAGCTTGACTCCCTGGAGCTCTGACGAACCTAGGATCTCAGGATAACAGGATCTCAGAATCACAGAGACA
GCTGAACTCTGAGGAGTTCTGACACAACAAGGATCACAAGAAGGACAGGATCCAGTCAGATATATGAGAGCAGATAGCACTAGAGAT
AATCAGATGGCAGGAGGTAAGCATAAGACAATAAGCAACAGAAACAAAGGTTACTTGGCATTATCAGAACCTAATTCTCCCACCATA
GCAAGTCTATACCATCACACCAGAAAAGCAAGATTCGGATCTAAAATCACTTCTCATGTTGGTGATAGAAGGGCATAAATAACTCCC
TTAAAGACATACAGAAGAACACAGGTAAACAACTAGAAGCCCTTAAAGAGGAAACCAAAAAAAAAAAAAATCTCTTGAATTCTAGGA
AAACACAATCAAACAGCAAAGGAAATGAACAAAACCATTGAAGATCTAAAAATGGAAATAGAAAACAATAAAGACATCACAAAGAGA
GACAACTCTGGAGTTAGAAAACCTAGGAAAGAGATCAGGAACCATAGATGCAAGCATCACCAACAGAATACAAGAGCTAGAAAAGAG
AATCTCAGGTGCAGAAGACACCATAGAAAACATTAACACAACAGTCAAAGAAAATACAAAAAGCAAAAAGCTCCTAACCCAAAACAT
CCAGGGAATCCAGGACACTATGAGAAGATGAAACCTAGGGATAAAAGGTATAGAAGAAGGTTTCCAACTTAAAGGGCCAGTAAATAT
CTTCAACAAAATTATAGATGAAAACTTCCCTAACCTAAAGAAAGAGATGCCCATGAACATACAAGAAATCTACAGGACTACAAATAG
ACTGGGCCAGAAAAAAAATTCCTCTCATCACATAATAATCAAAATACCAAATTCACTAAACAAAGAAAGAATATTAAAACAGTAAAG
GAAAAGGGTCAAGTAACATATAAAGGCAGACCTATTAGAATTACACCAGATTTCTCACCAGAGACTATAAAATCCAGAAGACCCTGG
GCAGATGTCATACAGACCCTAAGAGAACACAAATGCCAGCCCAGCCTACTATACCCAGCAAAACTCACATTACCATAGATGAAGAAC
CAAGATATTCTAATACAAAACCAAATTTACACAATATCTTCCCATAAATCCAGCTCTACAAAGGATAATAGATGGAAAACATCAACA
CAAGCAGGGAAACTACACCCTAGAAAAAGCAAAAAAGTACTCAACTTTCAAAAAACCTGAAAGAAGATAGCCACACAAACATAATTC
TACCTCTAACAACAAAAATAACAGAAAGTAACAATCACTTTTCCTTAATATCTCTTAACATCAATGGGCTCAATTCCCCAATAAAAA
GACATAGACCAACAGACTGGCTACATAAACAGGACCCATTTTGCTGCATACAGGAAACCCACCTCAGGAACAAAGACAGACACTACC
TAAGAGTAAAAGGTTGGAAAACAATTTTCTAAGCAAATGGTCCCAAGAAACAAGCTGGAGTAGCCATTCTAATACTGAATAAAATCA
ACTTTCAACCAAAAGTCATCAAAAAAGATGAGGAAGGACACTTCATACTGGTCAAAGGAAAAGTCTACCAAAATGAACTCTCAATTG
TGAACATGTATGCTCCAAATGCAAGGGCACCCACATTCATAAAAGAAACTTTACTACAGCTTAAACCACACATCTCACCCAACAATA
TAATAGTGAAAGACTTCAACACTCTCATCAATGGACAGATCATGGAAACAGAATCTAACCAGAGACACAGTGAAACTAAACAAAAGT
TATGAGCCAAAAGGATCTAACAGATATTTATAGAATATTCATCCTAAACCAAAAGAATATACCTTCTTCTCAGCACCTCTCAGTAAC
TTCTCCAAAACTGACCATATACTTGATCCTAAAACATGACATACAAGACATACAAGAACATTGAAATAATCCCATGCACCCTATTAG
ATTACCATAGACTAAGTCTGGTCTAGAATACCAACAAAAACGATGGAAAGCACACATACACATACAAGCTGAACAATGCTCTACTCA
ATGAAAACTTGGTCAAGGAAGAAATAAAGGAAGAAATTAAAGGCTTTTTAGAACTTAATGAAAATGAAGACACATCACACCAAAACT
TAGGGGACACAATGAAAGCAGTGGTAACAGGAAAACTCATAGCTCTGAGTGCCTCCAAAGAAAAACTGGAGAGAGCTTACCCCAGTA
GCTTGACAGCACACCTGAAAACTCCAGAACAAAAAGAAGCAAATATACCCAGGAGCAGTGGAAGGCAGAAAATAATCAAACTCAGGG
CTGAAATTAACCAAGTAGCAACAAAAAGAACTATATAAATAATCAACAAAAGCAGGAGTGGGTTCGTGGTGGCCCATGCCTTTATTC
CCAGCACTTGGGAGGCAGAGGCAGGCAGATTTCTGAGTTCGAGGCCAGCCTGGTCTACAGAGTGAGTTCCAGGACAGTCAGGGCTAC
ACAGAGAAACCTGTCTCAAAAAACAAAAAGAAAAAAAAAGAAAAAAAGAAACTCAACAAGATAGATAAACCCTTAGCCAAACTAACC
AGAGGGCACAGAGATAGTATCCAAATTAACAAAATCAGAAATAAGAAAGGAGACATAACAACTAAACATATCTTTAATTATTCTGTT
TGTTGAATATTAACATTTGAAAACATTAAAATCATGTTCTACACACATCATGGAAGTATTATTGACAATTTTTCGCACTGAGCTTGA
ATTAGCATTTTCTTAATGTGTAACTTCAAAGGGTTTTTGCTATTTTGAAATTTTTAAAATATACTTACTGATAAAATAATTTCTCTC
CTAGAGACACTGGTCTTTTCTAAGTAAACTTATAGTTAGACAATGTACACAGATATATAAAGCATTTTAAATACTCTCTCACTATGT
CAGGTGGTATCATATAAGGGCTTTTGAATGTATTTCTTGAGTCATTTTTAATATTTTGTGTTCTTTTATTGTGCTTAATTCCAATAT
TTTGTATGTTTTGAAACAATTTAGTATTCCACATTAGATATAGGATCCTCAATTATGGATAGAATCAAATATTCATTAACAATATTT
TTAGGGTATGAAAGGATATGAATATAAAAGTTGTACAATTTTTTATGTATTATTTGATTCTCAAAATACTCAATATTATTAATATGT
TTGATGTATAAAATGCATTTAAATAATAAATATTTAAGGGAAAAACTAAATAAACAAAAACAAAACAAGGAGATACAGGAGATAGCA
AATAGACAAGGAAGAAGTTAAAAGGATGTGTTAGCCCATTTTTTTTGGTAAGAGACATGGAAAAATATGTTAAATATGTTAAATTAT
TCAAACTATCACCTATATCAGACTATTTTTCAATTTACTTAATTATATACAATGCAAAAATGAGCTTTATTTCTGCTGAGTGAAGGT
TCTAGAGTTGTATTATCACGTGATTCATTTTCTCCCTGTTCTGACAGGTACACATGCTAGACTCTCCACCAGGCACACACGCTTTGA
GGAACAAGTTTATAACATTCCAATCCGCATCAATGATGGGGGGCAGCCACCCATGGAAGGGACTGTCTTCTTACCAGGTAGGTACCG
TGTAAAGGTGATGTAGGATATAGGGTACCATGTGCACTTTAAGAAATGCTTCTTTAAGTTGGGATTTTGCAACTATTCACTTAAAAA
CGTAATCTATGGAGCCCTGTTCTGCTTTTTAGTTACTTTCTGCCAGTGTGTGGAAGGAAGCTGTTTCCGGCCAGCAGGCAGACAGGA
TGGGATACCCACTGTGGGCATGGCAGTTGGTATACTTCTGACCACATTTCTGGTCATTGGTAAGTATGACTTTTCAGAGCCAAGGTT
TCCTGCCCCCAGCCTCAGATGTGAGCTCATCATGCCATCTATAGGGGTGCTCTGGGGGTTTCTTGCCTTGGGTATCAACCAAGAATA
CAAGTCCCCAGTCAATCACATCAAAGTATCCTGGTTGAGAAATACTGTGTCAAAGCTCCCATCTTCCTGTCATTTCCTTGCTTGGAT
TTTCATGCTGACATTTATACCAGAAGGCTCTTTATTAACCAGCCATTCTTTATTGTAGCAAATATCTAAGACAATCAACATTTTAAA
AAAGTTTCTATTTGGCTCATGGCTTCAGTAGTTTCAGAGGTCCACGATTGACTGGCCTGTTGCTTGGGGGTCCACAGGAAGATAGTA
CTTTATTGAGGAAAGCTGCTCACCTCCCTTTGGGAACAAATGAGAGAGGAAGCGGGAAGGGGCTCAGGTCCTACTGTTCCCTTGCAG
GGTACAGTCTTCAACTGTCTAGGGGAGGTCGTCTGTTGATAATGCTGAATTGGGAACATTTGAAGGCCATTTAAGATTGTAACCAAT
ATCAAACTTTACACACAAATTGCTGCATTTTGGGCCGAGTTACCAAGCTCTTATGAAGGAGAAACAAATTAGTCTCTGTGACCTGCT
GTGTCGGGCACTGAGTTTATTAGAACTGATATCATGATGGGCAAGGCCTGCATTGACCCTCTACCCGTGTTGGAATAGGGAGGGGTA
GGCTGTGAAATGAAGATTCTGTGACACCTGAGATGCTGCCGAGTTTCCCTTTGCGTTATTTTCATAACTCTGGCCTCTGCGTCTTTA
CAACCTGGAGTAATTTCACAGCATCATGGCTTTGACTCTTCCCCTAGCTGGAGAGCTGTCCTCTCTCATGCACGGAGAGCAGCTGGC
CTCCTTGAGAACGCTGTCTGCTGCATGTGAGCGGCCCTTCTTTTGAGAGGGACTTGCTTTGTAGTCCAGACTGGCCTTGAACTCAGG
CAAGCTCCCAGCCTCAGAGCTAGATTTTGAGATACTGTTTACTAAATCCTTTGGCCTTTATGCCTTTTATTGTTTTTCCTACCCACC
TTGAGAAGATTGAGTTCATGCGCCACACTGAAATTTTTTCACACAGCCTCATTCCCATGATCCTACTCTAAGTAAAGAATGGGCAGT
TTTGAGGATATATAGAAATAGAATTGACAGGGCTATTTTTGTTTTGTACCCTTATTCTTTTCGAATGGGGGCATGTGAATAATCACA
AGATTGTCAGCAAGTGTCTGAATGTCTAGTCTTCTTGGCTATGGTGCCTTCTGTAAGCTTATTGATGAGCAATTTCCTTATAGTAAC
TCTACACTTTTCCTAGTGGGCTCTGACAGTTGAGATTAGTGCTACAAAGCACAGCGTAGTCCTGTTAGAGTTCAGAGAAGTAAGAGA
AACCAGAATGAGTAGGCAGGCCACTGAAGGTCCCTCAGGAGAGGCTGGTACTGCGGGGAAGGTCCCCAAGCTGCGCATCTGACAACT
GCTCTACTAGTAAGCTGCATGCTGAGCCCTTACTGTGAAGTTAAAATTGTTTCAAAATCAATTTTAAATCATAAACAATTCATATTT
GGTAGATACAATGAAAGTAGACTCTAGGCAGGTGGGCAGGTCTAGATAACTCCCTAGTTATGATTAGACTTTTTTCTCTAGGGAAGA
AAAACTAGCATCAATGTTTGGGTATATACGTGGTTCAGACAGACTTATTTCCACATTCTTTTCCCTCCGAGGAATTATAGCACTGGC
TCCTCTATTCCCTATTGCCAGCCTCCAGGTTAGTTATAACTTCGGGCTTCCTATATACTCATAACAAATGACCTCTTTACTTATCCA
AGAGAGCCAATGCCCAGAGCTCTATACTCACTAGAACAACGAAGCACAGTTTAATCACACAAATGGTGGCTCACAAGTCATCCCCAC
TCCTACCCTATAAGTGACTGTGTTAGGACATCTGAAGACTGTGCTTGTTTGACCCTGAGTTTTCTCTTTTCAGGTATAATTTTAGCA
GTTGTCTTCATCCGCATGAGGAAAGATAAAGTTGAAAATCCGCAGTCCCCTGAGAATAAGCCTCTGAGAAGCTGAATTTGAAAAGAA
ATGGTCAAGTCTGTGCACCAAGCACTATATCAATGCCTCAGCTGTTTAATTTCATCCAAAATGTGAACTATAATTGGTAGAGTTTAT
GCATCATGGGCTATATCTGGTCCCCAGCTGTTTAATCTCATAATTTCATCCAAAATGTGAACTGTAATTTCTTTGATGGCATGCCCT
TCTTGTCCTGCAACATTTTATGCTACTTAGATATTTTATGTTCTGCAGACATTAGAGTTATTCTCCATTTCCCCATAACATTTTCCT
CCTTTACAAAAGCCTTAGCTTTATAACTTGAACCTTCCCCTCTGGGCAGGAAGAGGAGCTGAGTACACACCCTGTGTTGTTGCTTCA
GAGGACGTAGCTGTTATCTATCACCAAATTTCCTAGGTTCCACTTATCCTTTCTGTTTCAGCCTGTGCTCCTTCATCTCTTAGCATT
ACACTGAATTTAAAATGTGTCAAACAGAAGAACAAAGTGAAGTCCTGGTTGGGGGTTGGGGGGTAAGTGGGAGCCTTGTTCTTTAAC
ATGGGCAACAGCTTCTCTGTGCTGTTAATGTTGTTAATGGCGGTCCTCCAACTTGGCTATGGCACAGACGCTGATGAGGTTATCAAA
TTCAACTGCTCTATTTCAAGAAAGATCTACTTCCTACGGCACAGGAACTGGTGAGCTGTCTGGTTTACTCACTACCACATGCTTTCA
TACCTGCTGTACACGTTTTATTTGTATATTGAAGTTTTGTTATATATTTATCATGTGGAGGAAACAAGAAAGTATAAAGTGGTAAAG
AAATC
MOUSE mRNA SEQUENCE: mR28-010.1 (Seq ID No: 56)
AGGGAGAGGCTAGAGGAGCAGAACAGGTTGAGGAAGTGTCTCTTAGGGTTCTTCAGCCACAGCAAGTGTCTCCACGGCTGAATGGAC
GACAAAACTTCTAAGCAGCATTTCTTACTTACTGAAAGGAGTCCAAGTGAAGAGAGCAAGATGGTGTCTGCCCAGCTTCAACTTCCT
GTGTCTTCTCACACTTTATTTGACCTGTGGATATGGCGAAGAAGGGAAGTTCAGCGGTCCCCTGAAGCCCATGACATTCTCCATTTT
TGAAGGCCAAGAACCGAGTCAAGTCATATTCCAGTTTAAGACCAACCCTCCAGCTGTGACTTTCGAGCTAACGGGAGAGACAGAAGG
TATATTTAAGATAGAAAAGGATGGACTTCTGTATCACACAAGAGCCCTGGACAGAGAAACAAGAGCGGTTCATCATCTGCAGCTTGC
AGCCTTGGATTCTCATGGAGCTATAGTGGACGGTCCAGTCCCCATCACCATAGAAGTCAAGGACATCAATGACAACCGACCCACGTT
TCTCCAGTCAAAATATGAAGGCTCAGTGAGGCAGAACTCTCGCCCAGGAAAGCCTTTCATGTATGTCAATGCTACAGATCTGGATGA
TCCGGCTACTCCCAATGGCCAGCTTTTTTATCAAATTGTCATCCAACTTCCCCAAATCAACGATGTTATGTACTTTCAAATCGACAG
CAAAACAGGGGCAATATCGCTTACCCCAGAAGGATCCCAGGAATTGGATCCAGTGAAGAATCCTTCCTACAACCTGGTGGTCTCGGT
GAAGGACATGGGGGGCCAGAGTGAGAATTCCTTCAGTGATACTACATATGTAGATATTTCTATTAGAGAGAATATCTGGAAAGCACC
AGAACCCGTGGAGATTAGAGAAAACTCGACTGATCCTCACCCCATCAAAATCACTCAGGTGCAGTGCAATGACCCAGGGGCCCAGTA
TTCCTTAGTCAACAAGGAGAAGCTGTCGCCGTTCCCATTCTCGATCGACCAAGAAGGAAATATTTATGTGACTCAGGCCTTGGACCG
GGAGGAAAAGAACTCACATGTTTTCTTTGCAACTGCCAAGGATGAGAATGGAAAACCACTTGCATATCCACTGGAAATTTATGTGAA
AGTTATTGACATTAATGACAACCCACCGACATGTCTGTCTCCAGTGACTGTATTTGAAGTCCAGGAGAATGAACCATTGGGTAACAG
CATCGGGATCTTTGAGGCCCATGATATGGACGAAGCTAACAACATCAACAGTATTTTGAAATACAAGCTTGTAGACCAAACACCCAA
AGTTCCCTCAGATGGACTTTTTCTCATTGGTGAATATGAGGGAAAGGTTCAGTTAAGTAAACAGTCCTTGAAGAAGCAAGACAGTCC
TCAGTACAACTTAAGTATCGAGGTGTCTGACGTAGATTTCAAGACTCTCTGTTATATTCAAGTCAACGTTATTGATATCAATGATCA
GATTCCCATCTTTGAAACATCAAATTATGGAAGCAAGACTCTGTCTGAAGACACCGCCATCGGATCCACCATCCTAATCATCCAAGC
TACAGATGCTGATGAGCCCTTTACAGGGAGCTCTAAAATCCTGTACAAGATTGTACAGGGAGACACGGAGGGAAGACTAGAAGTTGT
CACAGATCCCACGACCAATGCAGGATATGTCAAGATCAAAAAGCCTCTTGATTTTGAAACTCAACCAGTTAGCAGCATTGTGTTCCA
AGCGGAAAATCCTGAGCCACTGGTGAAAGGCATAGAGTACAATGCTAGTTCTTTTGCCTCCTTCGAGCTGATTGTGACGGATGTGAA
TGAAGTGCCTGTGTTCCCCCAGCGAATATTCCAAGCAAACGTCAGCGAGGATGCTGCTGTGGGCAGTAGAGTGGGCAACGTGACTGC
CAGGGATCCTGAAGGCCTGACTGTGAGTTATTCACTGAAAGGCAATATGAGAGGCTGGCTTAAAATCGACTCTGTCACTGGTGAGAT
ATTTAGTGCTGCTCCGCTGGATAGGGAAACAGAAAGTGTGTATCGAGTACAAGTGGTGGCCACTGAAGTAGGTGGGTCCTCTCTGAG
TTCTACGGCAGATTTCCACCTGGTCCTCACAGATGTGAATGACAACCCCCCTCGCCTAGCTAAGGACTACACAGGCTTGTTCTTCTG
CCATCCCCTCAGTGCCCCTGGGAGCCTCATCTTTGAGGTCACTGACGATGACCAGCAGTCACTTCGGAGGCCCAAGTTTACATTCGC
CCTTGGCAGAGAAGGCTTACAAAGTGACTGGGAAGTTTCAAAAATCAATGGTGAGTTATCAAATGTACAATGAGAAAAATCAATGGT
GGATGAAGGAATTACTTTGTGTGTGTATGTGCTAAAAACTTGAAGCATTTTCACTAAAATCAGGAATAAACCAGGAGTGCCCCACCC
TCCCTGACACCTGTTCAGTATAATAACTTGTAACATTAGATAAAAAATAACTCTGCAGCCAGTCCCACAGCACCCAGAGGAACCTCC
ACTCACAGGCACTCTAACAATCCCAGGATCATAGGACCAGAGGTGAAGAGGACACAACATCTACCCCAACATTGGAGGTAACTGGGA
CCAGCCGGACACAGGCATCCAGGAGCTCTGCCCACATGTGACACAGGTTACTTCCTCCCTGGGCTGGTGTCCTGAGCAGAATTTTGG
TTCAAACTCCCCAGCCAGTTTCACAACACCCAGAGGAAGCTCCACTCCCAAGGGCTCTGGCTCTCCCAGGATCATAGGATCAGAGGT
GCCCTGAGCAGACCTTGGGTGTGAACTCCGGAGCCAGTACTACAACACCCAGAAGTTTGACTCCCAGGCACTGTAACACCCCCAGGA
CCACAAGATCCCAGGATCCCAGGATCCTAGGAGCTTGATCACACTAGGATCTTAGGGTCCCAGAGGCAGCTTGACTCCCTGGAGCTC
TGACGAACCTAGGATCTCAGGATAACAGGATCTCAGAATCACAGAGACAGCTGAACTCTGAGGAGTTCTGACACAACAAGGATCACA
AGAAGGACAGGATCCAGTCAGATATATGAGAGCAGATAGCACTAGAGATAATCAGATGGCAGGAGGTAAGCATAAGACAATAAGCAA
CAGAAACAAAGGTTACTTGGCATTATCAGAACCTAATTCTCCCACCATAGCAAGTCTATACCATCACACCAGAAAAGCAAGATTCGG
ATCTAAAATCACTTCTCATGTTGGTGATAGAAGGGCATAAATAACTCCCTTAAAGACATACAGAAGAACACAGGTAAACAACTAGAA
GCCCTTAAAGAGGAAACC
MOUSE PROTEIN SEQUENCE: mP28-010.1 (Seq ID No: 57)
MVSAQLHFLCLLTLYLTCGYGEEGKFSGPLKPMTFSIFEGQEPSQVIFQFKTNPPAVTFELTGETDGIFKIEKDGLLYHTRALDRET
RAVHHLQLAALDSHGAIVDGPVPITIEVKDINDNRPTFLQSKYEGSVRQNSRPGKPFMYVNATDLDDPATPNGQLFYQIVIQLPQIN
DVMYFQIDSKTGAISLTPEGSQELDPVKNPSYNLVVSVKDMGGQSENSFSDTTXTDISIRENIWKAPEPVEIRENSTDPHPIKITQV
QWNDPGAQYSLVNKEKLSPFPFSIDQEGNIYVTQALDREEKNSHVFFATAKDENGKPLAYPLEIYVKVIDINONPPTCLSPVTVFEV
QENEPLONSIGIFEAHDMDEANNINSILKYKLVDQTPKVPSDGLFLIGEYEGKVQLSKQSLKKQDSPQYNLSIEVSDVDFKTLCYIQ
VNVIOINDQIPIFETSNYGSKTLSEDTAIGSTILIIQATDADEPFTGSSKILYKIVQGDTEGRLEVVTDPTTNAGYVKIKKPLDFET
QPVSSIVFQAENPEPLVKGIEYNASSFASFELIVTDVNEVPVFPQRIFQANVSEDAAVGSRVGNVTARDPEGLTVSYSLKGNMRGWL
KIDSVTGEIFSAAPLDRETESVYRVQVVATEVGGSSLSSTADFHLVLTDVNDNPPRLAKDYTGLFFCHPLSAPGSLIFEVTDDDQQS
LRRPKFTFALGREGLQSDWEVSKINGELSNVQ*
MOUSE PANTHER CLASSIFICATIONS
FAMILY (SUBFAMILY)
CADHERIN (LIVER-INTESTINE-CADHERIN)
BIOLOGICAL PROCESS
Signal transduction(2.11.00.00.00) > Cell communication(2.11.03.00.00) >
Cell adhesion-mediated signaling(2.11.03.01.00)
Cell adhesion(2.29.00.00.00)
MOLECULAR FUNCTIONS
Cell adhesion molecule(1.05.00.00.00) > Cadherin(1.05.02.00.00)
MOUSE GENE ONTOLOGY
BIOLOGICAL PROCESS
cell adhesion > homophilic cell adhesion
MOLECULAR FUNCTION
ligand binding or carrier > calcium binding
cell adhesion > calcium-dependent cell adhesion
CELL COMPONENT
cell > membrane fraction
MOUSE PROTEIN DOMAINS (INTERPRO SIGNATURES)
IPR002126 (CADHERIN)
IPR002126 (CA)
IPR002126 (cadherin)
IPR002126 (CADHERIN 2 5)
MOUSE mRNA SEQUENCE: mR28-010.2 (Seq ID No: 58)
AGGGAGAGGCTAGAGGAGCAGAACAGGTTGAGGAAGTGTCTCTTAGGGTTCTTCAGCCACAGCAAGTGTCTCCACGGCTGAATGGAC
GACAAAAACTTCTAAGCAGCATTTCTTACTTACTGAAAGGAGTCCAAGTGAAGAGAGCAAGATGGTGTCTGCCCAGCTTCACTTCCT
GTGTCTTCTCACACTTTATTTGACCTGTGGATATGGCGAAGAAGGGAAGTTCAGCGGTCCCCTGAAGCCCATGACATTCTCCATTTT
TGARGGCCARGAACCGAGTCAAGTCATATTCCAGTTTAAGACCAACCCTCCAGCTGTGACTTTCGAGCTARCGGGAGAGACAGATGG
TATATTTAAGATAGAAAAGGATGGACTTCTGTATCACACAAGAGCCCTGGACAGAGAAACAAGAGCGGTTCATCATCTGCAGCTTGC
AGCCTTGGATTCTCATGGAGCTATAGTGGACGGTCCAGTCCCCATCACCATAGAAGTCAAGGACATCAATGACAACCGACCCACGTT
TCTCCAGTCAAAATATGAAGGCTCAGTGAGGCAGAACTCTCGCCCAGGAAAGCCTTTCATGTATGTCAATGCTACAGATCTGGATGA
TCCGGCTACTCCCAATGGCCAGCTTTTTTATCAAATTGTCATCCAACTTCCCCAAATCAACGATGTTATGTACTTTCAAATCGACAG
CAAAACAGGGGCAATATCGCTTACCCCAGAAGGATCCCAGGAATTGGATCCAGTGAAGAATCCTTCCTACAACCTGGTGGTCTCGGT
GAAGGACATGGGGGGCCAGAGTGAGAATTCCTTCAGTGATACTACATATGTAGATATTTCTATTAGAGAGAATATCTGGAAAGCACC
AGAACCCGTGGAGATTAGAGAAAACTCGACTGATCCTCACCCCATCAAAATCACTCAGGTGCAGTGGAATGACCCAGGGGCCCAGTA
TTCCTTAGTCAACAAGGAGAAGCTGTCGCCGTTCCCATTCTCGATCGACCAAGAAGGAAATATTTATGTGACTCAGGCCTTGGACCG
GGAGGAAAAGAACTCACATGTTTTCTTTGCAACTGCCAAGGATGAGAATGGAAAACCACTTGCATATCCACTGGAAATTTATGTGAA
AGTTATTGACATTAATGACAACCCACCGACATGTCTGTCTCCAGTGACTGTATTTGAAGTCCAGGAGAATGAACCATTGGGTAACAG
CATCGGGATCTTTGAGGCCCATGATATGGACGAAGCTAACAACATCAACAGTATTTTGAAATACAAGCTTGTAGACCAAACACCCAA
AGTTCCCTCAGATGGACTTTTTCTCATTGGTGAATATGAGGGAGGTTCAGTTAAGTAAACAGTCCTTGAAGAAGCAAGACAAAGTCC
TCAGTACAACTTAAGTATCGAGGTGTCTGACGTAGATTTCAAGACTCTCTGTTATATTCAAGTCAACGTTATTGATATCAATGATCA
GATTCCCATCTTTGAAACATCAAATTATGGAAGCAAGACTCTGTCTGAAGACACCGCCATCGGATCCACCATCCTAATCATCCAAGC
TACAGATGCTGATGAGCCCTTTACAGGGAGCTCTAAAATCCTGTACAAGATTGTACAGGGAGACACGGAGGGAAGACTAGAAGTTGT
CACAGATCCCACGACCAATGCAGGATATGTCAAGATCAAAAAGCCTCTTGATTTTGAAACTCAACCAGTTAGCAGCATTGTGTTCCA
AGCGGAAAATCCTGAGCCACTGGTGAAAGGCATAGAGTACAATGCTAGTTCTTTTGCCTCCTTCGAGCTGATTGTGACGGATGTGAA
TGAAGTGCCTGTGTTCCCCCAGCGAATATTCCAAGCAAACGTCAGCGAGGATGCTGCTGTGGGCAGTAGAGTGGGCAACGTGACTGC
CAGGGATCCTGAAGGCCTGACTGTGAGTTATTCACTGAAAGGCAATATGAGAGGCTGGCTTAAAATCGACTCTGTCACTGGTGAGAT
ATTTAGTGCTGCTCCGCTGGATAGGGAAACAGAAAGTGTGTATCGAGTACAAGTGGTGGCCACTGAAGTAGGTGGGTCCTCTCTGAG
TTCTACGGCAGATTTCCACCTGGTCCTCACAGATGTGAATGACAACCCCCCTCGCCTAGCTAAGGACTACACAGGCTTGTTCTTCTG
CCATCCCCTCAGTGCCCCTGGGAGCCTCATCTTTGAGGTCACTGACGATGACCAGCAGTCACTTCGGAGGCCCAAGTTTACATTCGC
CCTTGGCAGAGAAGGCTTACAAAGTGACTGGGAAGTTTCAAAAATCAATGGTACACATGCTAGACTCTCCACCAGGCACACACGCTT
TGAGGAACAAGTTTATAACATTCCAATCCGCATCAATGATGGGGGGCAGCCACCCATGGAAGGGACTGTCTTCTTACCAGTTACTTT
CTGCCAGTGTGTGGAAGGAAGCTGTTTCCGGCCAGCAGGCAGACAGGATGGGATACCCACTGTGGGCATGGCAGTTGGTATACTTCT
GACCACATTTCTGGTCATTGGTATAATTTTAGCAGTTGTCTTCATCCGCATGAGGAAAGATAAAGTTGAAAATCCGCAGTCCCCTGA
GAATAAGCCTCTGAGAAGCTGAATTTGAAAAGAAATGGTCAAGTCTGTGCACCAAGCACTATATCAATGCCTCAGCTGTTTAATTTC
ATCCAARATGTGAACTATAATTGGTAGAGTTTATGCATCATGGGCTATATCTGGTCCCCAGCTGTTTAATCTCATAATTTCATCCAA
AATGTGAACTGTAATTTCTTTGATGGCATGCCCTTCTTGTCCTGCARCATTTTATGCTACTTAGATATTTTATGTTCTGCAGACATT
AGAGTTATTCTCCATTTCCCCATAACATTTTCCTCCTTTACAAAAGCCTTAGCTTTATAACTTGAACCTTCCCCTCTGGGCAGGAAG
AGGAGCTGAGTACACACCCTGTGTTGTTGCTTCAGAGGACGTAGCTGTTATCTATCACCAAATTTCCTAGGTTCCACTTATCCTTTC
TGTTTCAGCCTGTGCTCCTTCATCTCTTAGCATTACACTGAATTTAAAATGTGTCAAACAGAAGAACAAAGTGAAGTCCTGGTTGGG
GGTTGGGGGGTAAGTGGGAGCCTTGTTCTTTAACATGGGCAACAGCTTCTCTGTGCTGTTAATGTTGTTAATGGCGGTCCTCCAACT
TGGCTATGGCACAGACGCTGATGAGGTTATCAAATTCAACTGCTCTATTTCAAGAAAGATCTACTTCCTACGGCACAGGAACTGGTG
AGCTGTCTGGTTTACTCACTACCACATGCTTTCATACCTGCTGTACACGTTTTATTTGTATATTGAAGTTTTGTTATATATTTATCA
TGTGGAGGAAACAAGAAAGTATAAAGTGGTAAAGAAATCGGAAAAAAAATGTTGGTTTCAATT
MOUSE PROTEIN SEQUENCE: mP28-010.2 (Seq ID No: 59)
MVSAQLHFLCLLTLYLTCGYGEEGKFSGPLKPMTFSIFEGQEPSQVIFQFKTNPPAVTFELTGETDGIFKIEKDGLLYHTRALDRET
RAVHHLQLAALDSHGAIVDGPVPITIEVKDINDNRPTFLQSKYEGSVRQNSRPGKPFMYVNATDLDDPATPNGQLFYQIVIQLPQIN
DVMYFQIDSKTGAISLTPEGSQELDPVKNPSYNLVVSVKDMGGQSENSFSDTTYVDISIRENIWKAPEPVEIRENSTDPHPIKITQV
QWNDPGAQYSLVNKEKLSPFPFSIDQEGNIYVTQALDREEKNSHVFFATAKDENGKPLAYPLEIYVKVIDINDNPPTCLSPVTVFEV
QENEPLGNSIGIFEAHDMDEANNINSILKYKLVDQTPKVPSDGLFLIGEYEGKVQLSKQSLKKQDSPQYNLSIEVSDVDFKTLCYIQ
VNVIDINDQIPIFETSNYGSKTLSEDTAIGSTILIIQATDADEPFTGSSKILYKIVQGDTEGRLEVVTDPTTNAGYVKIKKPLDFET
QPVSSIVFQAENPEPLVKGIEYNASSFASFELIVTDVNEVPVFPQRIFQANVSEDAAVGSRVGNVTARDPEGLTVSYSLKGNMRGWL
KIDSVTGEIFSAAPLDRETESVYRVQVVATEVGGSSLSSTADFHLVLTDVNDNPPRLAKDYTGLFFCHPLSAPGSLIFEVTDDDQQS
LRRPKFTFALGREGLQSDWEVSKINGTHARLSTRHTRFEEQVYNIPIRINDGGQPPMEGTVFLPVTFCQCVEGSCFRPAGRQDGIPT
VGMAVGILLTTFLVIGIILAVVFIRMRKDKVENPQSPENKPLRS*
MOUSE PANTHER CLASSIFICATIONS
FAMILY (SUBFAMILY)
CADHERIN (LIVER-INTESTINE-CADHERIN)
BIOLOGICAL PROCESS
Signal transduction(2.11.00.00.00) > Cell communication(2.11.03.00.00) >
Cell adhesion-mediated signaling(2.11.03.01.00)
Cell adhesion(2.29.00.00.00)
MOLECULAR FUNCTIONS
Cell adhesion molecule(1.05.00.00.00) > Cadherin(1.05.02.00.00)
MOUSE GENE ONTOLOGY
BIOLOGICAL PROCESS
cell adhesion > homophilic cell adhesion
MOLECULAR FUNCTION
ligand binding or carrier > calcium binding
cell adhesion > calcium-dependent cell adhesion
CELL COMPONENT
cell > membrane fraction
MOUSE PROTEIN DOMAINS (INTERPRO SIGNATURES)
IPR002126 (CADHERIN)
IPR002126 (CA)
IPR002126 (cadherin)
IPR002126 (CADHERIN 2 5)
HUMAN GENOMIC SEQUENCE: hD28-010 (Seq ID No: 60)
GGAAGAGGGAGTGTTCCCGGGGGAGATACTCCAGTCGTAGCAAGAGTCTCGACCACTGAATGGAAGAAAAGGACTTTTAACCACCAT
TTTGTGACTTACAGAAAGGTAAGGGCTGACATGTCTTAACTGTGTCAGTAACGTATTTATTCCAGAAGGACAAAGTAGATGGAGGGG
GAGGGCACTTAAAAAGTTGCTTGACAAATTTGTTCCTTGAGGTGGGCTATGCAGATGCGGAGAAGGGAAATTTGTTGCTGGCATGAC
AAGTTTTGCTTATGGAGCACAATTTTTTTTTGTAGTTTCTTTTCATTGGTCCATATTTTATAAATTCATTTGATGAAGTGGCTGCAG
TGAAATCAAGCTTGGCAGTGGAATTAAAGGGGAAATTGAGTTCTCTGATGTTTAAACAAGTATCACATCCATTCTTTGTGATGAGTA
TATAAAAGTCCCTGGTCTTAAGATGCTCTAGGGCTCTTGTATTCTAGAAATTGGGGTTGGAATCACATTCTCAAAAAGCATGTCATT
CTATCTACTTCTCAGAGAATGAGATTCAATTTGGGATTCTTAGAATTGTCTAGGACACAGCAAGGGAGGGAACATGCAGAGAAGAAG
AAAAGTCTTTTTGTAAAAGTACTAGAATAAGAATTGAAGACAGACAGACCTGCCTTCACAAGTGAGAGAAGAGGCTGATGTGCGTGT
TAATCAGCTTCCTGGGCTGCTTTTGGCCTTGGTGGTGGAGTTACCACCTGCTAATGTGTGGTTATTTTTATGTTAAGCCACACACAG
CTTATTGGTATCTTCAATGGTCCGATATTTGCCTCTGTCTTGGTCTTTTATGACAGGTGACTTTATAGGGACAGATGCCCTCTGTGG
TAGGGGATGCCAAGCAGCAGATTGGAAGGACAGTGCCTTCTCTAATATTTTCTTGTCTTTATTTTTCTTCAAAAGCTTAAATAGCAA
ATGAGGCATTATTTGAAAGAAGAAGAAAAAACACATGTTTAGATGAAAGGAAGATGAATGCTATTCAGCTCTCACCAGGAAACAAAG
CGAGTCCTTTACTGAGAGAGGGTTTCTAGTGAGTTACGGGGCCATATGAGTCTTCAGGGTTGGCCAAATGGGTTAGGCCAGTTTCAT
CATTTTCCAACCTGTGGGCACAGAACAGAGAGCTGCCACTAGAGACTTGCCTAACTAGGTAGTTGTGAGAAGTAAATTAGATAAGTA
GGTAAAGAGCTTACTATGTTGCTTGGTGCATGGTACACATCCAGTGAATGTTAGCGCTCTGTTCCATGAGAAAGTTCAAGTAGGTGA
CTCAGGATGAATTGAGAAATTTTTGTTTCATTCTTTAGAAAAATAAGAAAAAAATCAGTGATGCCATAAAAATAGCATCTACCATTA
TGCAAGAGAGGAACTAATTCTATGTTCAGGAAGTAGGATTCAAAAATATGAGAATAATAAAACTATTGGTTTTGCTATATATATAGC
ATTTTAAAGCCCCTCTCATGGTTCCCATATAAGTCTTTGGGTGAGGTGCAGGTGGGGCATCCTTTGAAACTCACCCAGATGACCCCT
GAAAAAAGTCATCACTGTTCAAGGTGAAGGTTACATTTCCGGTACCTCCTCCTTTCTCATACAAAAGGCAAAATATGGCTGGGTGTG
GTGGGTCATTCCTGTAATTCCAGTACTTTGAGAAGCTGAGGAGGGAGAACCGCTTGTGCTGGAGTTCAAGACCAGCCTGGAAGGTCT
GAACATAGGGAGATCCAGACTGTACAAGAAAATTAAAAATTAGCCAGGCATGATGGCACTTACCTATAGTCCTAGCGACTTGGGAGG
CTGAGGTGAGAGGATTCCTTAAGCCCAGGAGGTTGAGGCTGCAGTGAGTCATGATTGTGCCACTGCATTCCAGCCTGGGTGACAGAG
TGAGAACCTGTCTTCTAAAAAAAAAAAAAAAAAAAAAGGATGATGTGCAAAATATATCCTGGCAGAAACTCTACTTCTCGTTCCATT
GTCAAAAAAAATGTCTAGAAGGATGTGTCCCTCATTTTTCTTCCACATTAACAGCAACAGCTCTGAGGAGGAAAGCTGGGCATTGTC
ATGCTCCTTGCTCTGGAGGCCGACAAACAGTGCTTGAACAGAAGCTGGTTCACATGGGTAGAGATGTATAGATTTGACATAAAATTG
CATTGATTCAGAAATCTCTGGTATCAGATTTAGAATGGGGCTAGTTGTTCTGTAACTGCTCGTTGATTATCCAAAGAAAGACACACG
GAATTTTAAGGGAATACCTATCTACTTATTTGTTACTTGTAAGAACAGACAGGAAGAAGAAGGAATGTTTACTTAGGTACAAGAAAT
ATTAACTTAATAAAAGCAGATATTCCCCTAGTGGCTGAGGTATTAACTACACTAAAGTTGATTTTTTTTTTTCACTTAAGCAATACG
TAACAAGCTAAGCTATGCTTGATTTTTGTGTATGTGGTACAAAGAAAATTTCTCTTCAAATATTCTGTAAGACTGACATGGAATTCT
CTCATTCATGATATTTCTGAGTTATTCATTTTTGACCATATACTTATTACTTAAAAATATTAGGACTTGTTCTTAAGTGACGGATGA
GGAGAATGGAAATGGTAGATTTGTTCCCTTAACATCTAAGCTAAAGATGAGTTCTCTTCCTCACCAAGAGGCCATCTAGCAGAGTGG
CTTGGTGCTTGGCCCCTGCATTCACACCACTTCCTCAAGCGCTCTAAGCATCAGTTTCCTGCTCTGTGGGATAACAACAGGACCCAC
ATTATATGATTGTGTTGTTCAAGTAAGTTGTCCAAAGTGTTTAACTTAGCTTGCATTTGTTGAGCATTTGCTATTTTTTAAAACAAT
TCCAATGATTAAAAAAAAAAACAATTCATGAATGAGATTTCATCGTGGTCTGGCACCTTTTATTAAGCAGAATTAAATTTATTTTCC
CTTCCTCTCCTACCCATCCCAGTTGTCAGTTACGATTTAATTACAGAAATTGGAAAGTGAACCTGCCATACTTAAGCATGACTGCAC
ATTCTTGGCATTAAAATTCCTGCCTTGGGTCACTTTGCAAATGAACTGGTATTAGAGTATTTTTCTTTCTCTTCTCTTCTCTTTTCT
TTCTTTCTTTCGATCCTTCAGTGTCTAAATCCTATGGGATGGAACCTAATTTAGATTTACTTCCTACTCAATTTCTAAGGCCAGGAT
GTGTTGCTTTATTATCTTGACAAAATGCTCATCCGTAAACTTTATATTACATAAAGAAATGTTGTGTAATACATAAAGAAACACTTT
GTCAAAAAAAAGTCAAGCCACCCTTTATGAGAAATAGTGATTCCAGTCCTCCTCTTTAGTCCAGGGAAGATGCTTGTGATATTGTTC
TGCTAGATCTGGGGGAGAAAGGGTCTCCCCCTGCCACCTTCTAGGGTTTTTGCGGGTATATAGCTGCACACGTGAGGGAAGTCAATG
CTGAAATTCTTCCTGGCTACCTGCTTAAAACCCATTTATGCCTAGTGTTCCATTATTGGAACACTAAGCATGTGGGAGTTATTTATA
TCCTTCTGCACAAGGTCATCGTCAAGGTCTGATTGCAAAAATTCAAAAAATTGCAACCTCAGGTATAAATGGGTTACAACATCAGGC
AAGTGCCCTGGTGGGACAGTCACTGTTGTCCCTAGGTCAGTGGCTCCCTTTCCTGTGACTGCTCTGGCCCCCCTTCCATTCAATGCT
GGGATGTTTTGGGAGACTTGGAGGAGAGGCCTCTTCTCCCCAGCTGATCCTTCCCTTGCTCTGTCTTGGGGAGCACTGGGGGAACAA
ACGCCAACCAGGAACAACATGACACCTCTCTCAAATATTTCTTGGAACTTGTTATTCTTTTCTTTAATAAGAATAATTATGCTAGTA
ATCCCATTACTGGGTATATACCCAAAGGATTATAAATCATTCTACTATAAAGACACATGTACACGTGTGTTTATTGCAGCACTATTT
ACAATAGCAAAGACTTGGAGCCAACCAAAATGCCCATCAATGATAGACTGGATTAAGAAAATGTGGCACATATACACCATGGAATAC
TATGCAGCCATAAAAAAGAATGATTTCATGTCCTTTTCAGGGACATGGATGAAGCTGGGAACCATCATTCTCAGCAAACTAACACAG
GAACAGAAAACCAAACTCTGCATGTTCTCACTCATAAGGGGGAGTTAACAATGAGAACACATGACACAGGGGAACATCACACATCGG
GGCCTGTCCTGGGGTGGGAGGCAAGCGGAGGGAGAGTATTAGGACAAATCCCTAATGCATGTGGGGCTAAAACCTAAATGACGGGTT
GATGGGTGCAGCAAACCACCATGGCACGTCTATACCTATATAACAAACCTGCATGTTCTGCACATGTATCCCAGAACTTAAAGTAAA
ATTTTTAAAAAAAGAAAAAGTGAGACAAACATTTTTTAAAAAAAGAATAATTATGCTAGTAATGTGTATTATAATTGATTATATCAA
TCTAAACCATTTTAAGTTCTCCAGGGGACCATTTAGCCTCCCGTGAGCCCATCTTCCCTATCATGAACCTATTGGTAGAACAAATCA
TCACCTCTAATTTTGCATGTGTGTAAATCCAGATTTTCATATACTGAAAGATTGGCATATCTATATTACAGAGTTTAGTGACAGAAA
GCTGGGCTTATGTTTATGTATGTATGTTCATCCCTCCCTCCCTCCTTTCCTTCCTTCTTCCCTGTCTTCCTCTCTGCTCTAAAAACA
AATCATCCAGCTGCCCATTCCCTCCCCATGTGGAGTAATCTGTTTGAAGTGTGAGGCAGAAGCCCAGATTTATACCTGTCTGCTTGT
TTCTAGCTTATTAATGGCCACTTTAATTGGGTCCTTAATTTGTCAAAAGTGCAAATTGGTCAGATGCTTCTGCGGTTCTCTAAACCT
TGAGTGATCCAATTGGTGCCAGAGATCTGTGAGCCAGACCAAAAAGATGGGCGTGGCAGATCTGAACATTAATCGATGAAAAATGGT
TTGGGCGAGATTTTCATCTGATCTTTATATCTATCTGGCCTTGCCTTATAATTTATGCCGCTCTTAGCAATATACAGTGAGGAGGAA
AAATAAATGGCTCACAGGATATTTGGCACTGTTTGGTCCTGGTGGTGTTATGAATTCCAGTCACATCCCTTCCTGCCCACACTACCT
TAGGATATTTGAAAACAAATTCTGTAGCTCTCTCCTTCTGCAACTTCTTTGTTCTTTGAAGCTTCATGGCTCAGCCTTAGTGCTAGG
TGAATTATTTTAATTCAAAGCCCTTCATGCTTTTTAAAAAATAAATAAATAAGAGATGACTGACTCTAAGATTTGTCTATCACTTAG
CTTTTTTCACGGGGGACAAAAATCAATGCTTATGTGTTGACTTGTAAATCTGGAGATCTGGGACGTAGAAACTCCTTGTCCTTACTC
AAATAAATGGGTAAATGACAAGAGCGTCTAAACATCACCTTCCCATCTTGCAAGATGGTGGGTGAAAAAGTTGAGAAGCCGGATACT
AAGGAGAAGAAACCTGAAGCTAACAAGGCTTTGCCAGTGGCAAGGTGAAAAGGGGAACCTCAGAGCTAAAAAGCCCAAGAAGGGGAA
GCCCAAAATCCTGTCCTCATTGGAGGAATTGGCAGATATTCCTGATCTGCTATGTATTCCAGAAAGGCCATGTACGAGAGGAGGTAC
TTAGCTGCTAAACCCAAGATTGAAAAGAATCTCGCAACCGTTACAAACCCAGTTGAGGTGACAAGAATGGTGATGCCTGGCTATTTA
AACTTCACAAAATGCCTTGATATTATCCAACAGAGATGTGCCTCAAAAGCTGTCCAGCCATCGCAAGAAACTCTGCAGTCAGCATGT
GAGAAAACTTGGAGCCAGCACGGCCCTCGAGACCATTCTGCTCATCCTCCCTTGGTGCCACCAAGGCAGGAGGGTGGTTTTCCTGGA
GCAGCTGGGCAGTAGCTTGTTACTTGTGGCTGGACCTCTGGTCTTCCATGGAGTTCCTCTGTGAAGGACACACCAGAAATTTGTCAT
CACCACCTCCATAAAAATTGATATCAGGACTGGGTGCAGTGGCTCACACCTATAATCCCAGCACTCTGGGAGGTCAAGGTGGGTGGA
TCACTTGAGGCCAGGAGTTCGAGACCAACCTGGCCAACATGGCGAAACCCCCGTCTCCATTAAAAATACAAAAATTAGCCGGGTATG
GTGGCACACACCTGTAATCCCAGCTACTTGAGAGGCTGAGGCATGAGAATCACTTGAACCTGGGAGCTGGAGGTTGCAGTGAGCTGA
GATGGCGTCATTGCACTCCAGCCGGGCATCAGAGCAAGACTGTCTCAAAAAAAAAAAAAAAAATCGATACCAGCAGTGTGAGAAACC
CAAAACATCTTACCAATGCCTACTTTAAGAAGCAGTTGTGGAAGCCTAGTCAGTAGGAGGGTGAGATCTTTGACAGAAAAAGACAAA
TGTAAGACTACAAAGCAGTGCAAGGCTGATCAGAAAACCATGGACTGGCAAATTTTACCAAAACTCAAAGCTATTCCTCAGCTCCAG
GGCTACCTGCAATCTGTGTGTGCCCTGGTGAATGGAATTTATCCTCACAAATTGGTGTTCTAAATTTCTTAAGAAGAACCTAATTAT
GTAACTAAAAAAAAATAAATGAATGCCGTTAATCCCCTTGGGAGCTTGGAGCCTGGTGGGAGAATCAAGGAAGCCTTGGGTGGGCTG
CTGGGATGCGGGGGGTCCCTAGGTGTCTAATGACTGAGCAGAGCCCCCTGTGAGGCCACCTCTGTCCTGGGACAGCAGCACAGGCTA
AACAAAAGTTCCATGGGGTCTTCATTTGCTAGGGCTGCCTTAACAAAATACCACAGAACGGGAGGCTTAAACAGCAGATATTTACCA
TCTCAGTTCTGGTGACCAGAAGTCCAAAATCAAGAGGGCAGTAGGGTTGGTTCCTCCTGAGAGCTGTGAAGGAAGGATCTTCTGTGC
ACCTCTCTCCTTGGCTTGTAGATGGCTGTCTGTACATTCACACAGTGTTCTTTCTTTGTGGATGAGCCTGTCTCCAAATCTCTTCTT
AGAAGGACATCAGTCATTGTGGATTAGAGCCCACTCTAACAACCTCATTTTAACTTCGTTACCTCTGTAAAGACTCCAAATAAAGTC
ACATTCTGAGGTGCTTGGAAGTTAGGACTCCAACATATGCTTTTTGAGGGGGAGGGAGACACAATTCTACCCATAGCATTTGGATTG
ATTTAAAAAAGGGAAGATTCCTAAGGTGAAGCACAGAAATGAGGAGAAATAGTGAGACACAGAGATACATTTGCAGTAGAAACAAAA
CTTCCCAGACCAAGGCAGCCTTTTCCTCCAGGCAGGTCGGGGCAGGTCATGTCAGCATCTGAAAAAAAGAGCAGCTGCAAAGTAGGG
GGAAAAAACCTTTTCAACATTTTGGAAAGATCTGTAGTGGATTAAATGATACTTGGTGTCTCATCATCAGGATATTTGTGTTTCAGA
GGGAGTGAAAATTAGCCTGGCATTGTGACCTTGGGCAAGTCTCTTAATTTCTGTGGTCTTCATCGTTTGATTTTTCATTTCATCTTT
AAGAGGTATATTATAATAGCATCTGCTCCATAGAGTTGTTCTGAGAAATCAGCAAGATAGTTTTTCACCACCAAGTCTTATCGGTGC
ATGCAGCAAATTGTTTTCAGTCAATGTTTGCTGCCTTTGTCGTTATCCGGGCTGGATGCAGATCCAGGCCGCGGCGGGATTCCCATG
TCTTCTGTTGTGTGGTAGATTAAAATGAGGCACTGTTCCTGCAGAGCAAGCCCAGCAGCCCTGGTTCCCGTCTGCAAGCAGGTGCCC
TGGTCTACTGTTGAGCAAGTGGTGGTCATGGTGGTGCCCATTGGGTGAGGGGCAGCCACAGTTTGGGAACCGAGGCCTTCTTTTCAA
AACTCCTTTTTTTGTAAAAAAAAAAAAAAAAAAAAAAAAAAAAAAACAAAAGATAATAATAATAACCCTCTGATCTATGTTGCAAAG
GGTGTGCTTGGTCTCTCAAATAATTGCTGTAGTAACTGTCTGAATTTAGTCCTTGTATCTGAGGGAAATCCTGACAAACGGCAAAAA
TTCCACGCAGACGGTTGGCCAATTATTACAGGTAGTTCAGATAAAATGCCACAGAATCCTCTGGAAATCTATTTCAATTCTTTTCCA
AGTAGTGTTTTTGAGGAAACCTAGGTGTCTGGCTTGGTGGTTTCGGGCAATTTCAGTGATGAAGGATGGCTTCCCATCAGCACCATT
GCGACCCAGGGTACCCCACACTTGAATGGTATTGTGTCCATGGAAGGAGGAAGTGTAGGTGGGACCTGTTTGAGAGACCAAAGTCCT
CTTTTGACATAATCCTAGCACTAGTGTAGTTTGCCTTGAATCATTCGGGTAAAGGCTTTGCCTTGCTGCCTCATTTGGCTGTGAGGA
TGTTCCTGTGTAGGCTGTTTTTAGAATATTTTCATTAGTGGGCCACCAAAAGTTGGGAATTGTGAAGAGAAATCTCCTGTTGGACAA
TGTTTACAGTTTTGACTAAGCAAACGTGAGTGGCAAAATGAATTTTTGGCCAACAGTATTCCAAATATTAACTCGCTGAATCTCTTA
ACTCTCGAAGGTAGGTATTATTATTATTATCCCTGCTTTATAGATGAGTAGATAGGGCCACGAGTCAGCCGGTGGAGGAGTTAAGAC
CCAAGCCAGCCAGTCTAACTCCTGGGTCTATGCTCCTAACAACCGCATGGTGTGGTAGATTTGTCTTCAAATAAATCATGGCACCCT
ACGTGTGGCAATGGAAAGCAGTGACTTCTACTTCCCACTGGAAACCCTCGGTGAGGGGCAGGCCTAAATGTAAATGACAAAATAAAG
GATAAGTACATTTCTCCTTCTACTTCCTAGGTTCCATTGAAACATCCCAGACAGTGATCTCTCTGAGCATCTGACCAAAATAAGGCA
TTCCTTTTATTTTTATTTGCTTTTCTATCATTTAACTTGTGTTTTTCTGGAAGACTGAAGGTGTTAAAAAAAAAAAAATTAGTTCAA
GAAGAGATGATCTAGTTCGACCAGTTCTGGGTTATTTACTGAGCAGAAATCTGTTAGTGTATTCAAAGGAGAAACAACTCTTTTCAT
CAACAATAAAAATGTATTACACAATAAAGCTTTGAATGTTGAATGAGTTTCCTTTAATATATCATCTTTATTGAAAGCTCTTACAGG
TGAGAACAAATTAATAAGATATCCATGTTAGGTAATCATAAATGTTTTAGAATTTACCCAACATCTGATGATGGCTTGATATATGCA
AGGGATAGTATGAAGAGACCTGATAAGTTAAAAATAAGGCCTCTGCCCTTAAGGATTTTATTTAATAGTGGCAGTAATGCTAACAGA
CAAATGATAAAAAAAAAAAAATATATATATATATATATATATATATATATATATATATATATCAATCAGAACTGGCTCTGAGAGGAA
GAGACTGGCTGGAAATCAAGGAAGATTTCATGAAGGAAGTAGGGTTTAGGTCCTAGCTCCATGAAGATAGACCCTGTACCCCTTATT
TTGGTACCCCAGTTCTTGGCACGGTATCTGGGACATACTAGAAGTGGTGTAGTTCATTGCTATTTAAATAATTAAGTTGAACTTAGA
TGGTAAGATGTGAGAAGCAGAAATGAGGAAACATCTCCATCCAACCTCATTAAGCAAATATCCATTGTCCATTTTCTCTGGGCCAGG
CCTTGTGCCAGGCAGGGAGAGCAGCAGGGGTGAGAGCTTGGCTGCCAGAGAGCTCAGGGTGCAAAGTCCATCTGGGTGGAGGTGTGA
CTGGGTGTGGGAAGGGAGTGGCAGATAAGGCTGGAAGAGTTGGGAGCAGCATTTGAAAGATCTTGAATGCTGAGGAGTTTAAGGAGA
AAGATCTCCCAATGTGTCATTATTGCCTATATTTTTACTTGTATTAAGCAAATACAGGTTGATAGCTGACCGTGTACATTTTGCTAA
TATTACAGAAAACTAGTTTGTTAGACTTAATTTATTCATTTATAATTGATAATATCATCTTGCAATATGCTAGAATCTGAGGTTGTT
ACTTGTTTCTATGTGTTTCCAAACCAAATAAGTACTCTAAAGCAGTAATGTCTTGTTTAACTATGAGTTAATTCTAAAATGATGTTT
AGATATGTCAACCAAATTAATTATGTTGCAAAAGCTAATTTTTAAACATGCAAGGAATTTATGAATCTTGGAGTCCTGAAGGGTTTT
AATCCTAGCTTTTCCTTAAAATAGTTGTGGAATTTCAGGGCAAGTTGCTTAATTTATATAGCATGTATAAATATATATACATACATA
CATGATATTTGTATATCTCTACAGCTCTATCTCTGTCCATGCTCTATGTCATACATCTACATCTAGGTAGCTATCAAACTATATAAA
GCACAGTAGCAAGAGTTGAAATTCCAGAGTTTAAACTCTGGAATTGATAATCTGGTTGGAGGCAAGGTAATGTCACTTCCTAGCTGT
ATGTCTTTAGGCAAATTGCTTGACTTTTAACCTCAATTTTCTCACCTACAAAATGAGAAGAACAATTCCCAGCTCATTATATCTGAG
AAAATCATAGGAGCACAAACTTTTTCTTTTTTTCTTTTTTTTTTTTTTTTTTTAGACAGAGTCTCGTTCCATCACCCAGGCTGGAGT
ACAATGGCCCAGTCTCAACTCACTGCAACCTCTGCCTTCCAGGTTCAAGCAATTCTCCTGCCTCAGCCTCCCAAGTAGCTGGGACTA
CAGGCACCTACTGCCACTTCTGGCTAATTTTTGTATTTTTAGTAGAGACGGGGTTTCACTATGTTGACCAGGCTTGTCTCGAACTGC
TGACCTTGTGATCTGCCTGCCTCAGCCTCCCAAAGTGCTGGGATTACAGGCCTGAGCCACCGTGCCTGGCCAAGAGGACAAACTTAT
TATTGAGCAATGGCTATGTGTTTTCCATGTGAGGAGACTAAGGCACCGAAAGATGAAGTAGTATATCCATAGCCACATAGCTATAGA
GAGCAGTGCTGCGTTGCTAGGCCAGCTTTCTGACTCCAAGCCTACCTGCCATACCACTCTGTTACTGTTTTTATTTTTAATTTTAAT
TTATTATTTATTTTTATTTTTTGAGACAGGGTCTCGCTCTGTCACCCAGGCTGCAGTGCAGTGGCGCAATCATGGCTGGCTGCAACC
TCTGCCTCCTGGGCTCAAGTGATCCTCCCACCTCAGCCTCCCAGGTGAAGTGGCATCATTGTCTGGGATAAATACCCGAGGTCATCA
ACTCAGGGTAGAGAAATAGAAAACCTGGGCAAACAAGAAGTGAGTTTAAGAGCAGAGGTTTAATAGGCAAAAGAAAGAGAAAGCGGA
TTAGCTCTATCTCCTGCAGATAGAGGGGGGTGCCCGAGTGGGTCTTCCGGTTTTGTGGTGAAATGGCACAGGGTTTTATATATGAAC
TTGAGGAGGCGGTATCTGATTTACACAGGGCCGGAGAGATTGGTTGGACCAGGTGTGATGTTTGCATAGTGCTTGAAGAAGCTGGCC
TAATCTTTTATTATGCAGATGGATTCTCTACCTGGCTAGCACCATATAGTCTGTTGCTTACTGTACACGTGGTTACAAAGAAAAGGG
AAGATGGAGCCGCCGTGTTGAACATGCCCGGCCCCCAGATAGCCTTTTCCTATTGGCCGAGCTGCTGGCATTCACCTGTGGAAACTT
CCAGCTTGCTTATCTATGTCTGCAGCTGGATTCTACAGGCTGCTCTTTGTTAGAAATGATTTTGGGGCTGCTTTTTGTTAAAAGGAA
ACCTTACTGAGGACTCTCTTACCCTTACTATCTGCCTAAATAATTTATTTCCAGCTCCTGTATAACAAGTAGCTGGGATTACAGGCA
ATGAGTCACCCACCTGGCTAATTTTTGTGTTTTTAGTAGAGATGGGGGTCTCGCCATGTTGGCCAGGGTGGTCTCAAACTCCTGAGC
TCAAGCAACCCACCTGCCTCGGTCTCCCAAAGTGCTGGGCTTCTGGATGTGAACCATTGTTTCCCGCCTGTTACTGTTTTTAAAGAC
CTCTCTGCAAATCTACATCCTGGGTGATGCTTAATAAAGGATTGTTTCTTTATCTCCCAGAACATTGGTGAGGCTAAAATGAAATAG
CATTTTTACCCTTTAAAGTTCAAATATAAGTTATGCAAATGTAGGGCATGTAAAATTTTCAGGATCTAAATTTTTAGAGTGGTTAAA
ATGTTAGTGAAATTTTTTCCATGCTGGTGATATTAGTGATATGTGTTCAATGTAGAAAAACTTTAAAGAATAAGATGATCAAGTAAA
CCAAGATTATTCCTAATCTCTAGAGATAATAAAGTGATTGGAATTTTCGTGAAATAAATACATTGAAAAAATTTTCTTGACTCAGCC
AGGTGTGGTGGCTCACGCCTGTAATCCCAGCACTTTGGGAGGCCGACGCAGGCAGATCACGAGGTCGGGAGATTGAGACCATCGTGG
CTAACACGGCAAAACCCCATCTCTACTAAAAATACAAAAAATTAGCCAGGCGTGGTGGCAGGCGCCTGTAGTCCCAGCTACTTGGGA
GGCTGAGGCAGGAGAATGGTGTAAACCTGGGAGGCGGAGCTTGCAATAAGCTGAGATCGTGCCACTGCACTCCAGCCTGGGCGACAG
ACTGTTTTCCAGGCCATGTGGAAAATTTCTCTCACATCTTAAAATCAATTAGTCATGACATGTCTCATAGTGAAAGTCACAATCTCT
GTGAAATGGGAAGCCTTAAGGAGGAAGGGAAAAATAATATCGGACAGGAGACAATAAGGACAGAGTTCAGATTTTTGTTTGATCTCT
TTTGGAATAGTAAGAAATTAATTTTTTCTTAGGTTTATTGTGTAGGTAATGACAATATCTAGAAGATCAAAGAGGCAGCTGGTGACA
TCTTTGAATGAAGAATTGTGAGCATTCTTTAGTTCTTTTCACTTGCGGAATCTCATATGTTTGTGGCTTTGAATCAAAATGTTTAGT
TATCCTGGAATCTGCCAAAATACATCTTCTCCATCTGTATAATAGGAGTGAAAGGAATACTCATTGCTGGCTGGGCATCTGTAATCC
CAGCACTTTGGGAGGCCGACGCAGGCAGATCACCTGAAGTCAGGAATTCGAAACTAGCTTGGTCACCATGGTGAAATCCTGTCTCTA
CTAAAAATACAAAAAATTAGCCAGGCGTGGTGATGGGCATTTGTAATCCCAGCTACTTGGGAAGCTAAGGTGGGAGAACTGCTTAAA
CCCAGGAGGTGGAGGTTGCAGTGAGATGAGATTGCACCACTGCACTACAGCCTGGATGACAAAGTGAGACTGCGTTTCCAAAAACCC
CAAAACAAACACTCATTGCCCATCATTTAAGACGCTGAAATGAAGAAGATCATAACAAAGCAAAGCAGTTAGATAAATGGAATTATT
GTGAGCATGTTTTGAGATGTTGGCTTGCATATTGCTATGGATGATGTGCATGAAGCTTCTGGAAGACTGGGCTGCTCCATATAGAGG
AGAGGCTGGCCTTTTGGGATACCATTTTACTAGATTTATAACTGGATATCATCCATTGATTCTTACTGATTGGAAAGACATGATTTT
AACAGACTGGTAACTAACCATTTTTTTATCTGGTTCCTTCACAGGAATTTGAATAAAGAAAACTATGATACTTCAGGCCCATCTTCA
CTCCCTGTGTCTTCTTATGCTTTATTTGGTAAGAGAAGAGGGTGTTCTTCTCTATTTGTTTACTAGAATATTTTCTGCTATCTTTCT
ACCCCACACCAGGGAAAGAATGGCTATTATTACTTAAAAAAAGATGACCTATCAAAATTAAGTTGTTGTATAGAATGGAAACATAGC
TTGTTCCACTGTGATATTTTCGATGAAATGGCTACAAATCACATGTCTGCCCTCGACTAGGGAATGTATTTTCTTTCCAGCTCTTAA
AAGCCAACACCAGTTATGGGTAATGTTGAAAACAAATCTATCTATAGCTCAGGGAATCTGAAATGACATTGCACTGATACATGAATT
AGTCTGTGTCCCTTAATCTTGACTTTCTTTTTTAAACTTTGCGCATGAAAACTTACCACCAAAACATCAAGAAAAGCATTTTCTCTA
TTTTCAGCCTTGCTCTGCTCTATACTTTTCTTCTCATCAAGGGTTTGGAATTTTCTGGTTGTCTTTAACCATTTGTTGGGTTAACCC
TATTGACTGACAGGCTTCTAGAGCTCGGAGCTGGATAGAAACTGGGGAGTCCTCACCCTTGACCTTGGGGTAAACAGTAGGTGCCTT
TTTTTAAAATCTACGTGACTGCTGAGGTTCATAAAGGTTGCCCCCAAAATTGCTGCATTATGAAACGAGATTATAAGATTTAGAGGA
AGCATTTGCTCTCTTTTTTTTTTTTTTAAAGAAGGAAGCTTCTAGATCCTCATACCTCAAATCTTACTTCCTTTCTTCTGGGATCCA
TAAATAATACATCAAATTTCAAGTCCCAAAAAATACCCTAGAGCTCACATAGTGACAGTGTGAAATTGAATGAAACTTGAAAACTAT
CAGAGCCTTCCTCCCGGGGGAAATCTGCAGTTCAAACTTTATGAGAAGCCTGAGTCCTCACAGCCTGCAGGCTCAAAGCCTGTCTCC
AGGATTGTGGAAGGGCTGGATCAAACATCCCCACTTGACCTCTCTCCTCATGCTACCCCCTCAAATGCCCTTTGTCACCTATGTTAT
CCCAGGGTAGTGACAAATTCAGACATGGATTCAAACTCGGTGCCTCCACTACCAGTGATAAGAATTTGGTGAGTTACCTAAGCTTTG
AACCTCAGTTTTCTGCCCTGTGAAGTGGATCTAATAATAGTACTTGTCTTATGGGATTTTGTGGCAATTCAATGAGATACTGCATTT
ACAAAGCTCTTGGATCAGCGACTGGTGGTTAAACACACAATAAACATTAGCTACTATAATTACTACAGCATTTTAAGTGTTCAGTGT
GTACGTCCACATGGACTCTGACCTCCATGAAAAATGGACTGTGCTTTTATCATCTTTGCAGATTCAGCACTGAGCACACTGCTAAAT
TATTTAATAATAATATCTAATTATTAGGCCTTATTTGCTTCTTGTCTGAGAAGGGAACATGCATTTTTATTCCTGGATGAAAGGTGA
AAATAAGGTTTCCTTCTCTCTACTCTCACCCCAGTGGGATTTCTATCTAGAGTGAAAATACTGCCCAACCACTGCTGGCAGCAGTGC
CTTACCCAGGTGAAAGCCACAAGCACAGCTGTCTCTGGGTGAAGTCAGGGAGTGGGATTTCAGGGGTGTTGGGAGGGAGCCACTTCT
CTCTCCTACCTCCTCCCAAGGCCTCTTCTATGAAGACTGGAAGTTTGCTTGAAGAATCTTGCCTTCTGCATAGGGCTTTATTCAACA
GAAGCACTAATTTACTCCAGACTGCGAGTAGATCTTCTGTTCTTTTCCAAGAGATAGATTTATCAGAGGGTTAGAAACACAAATACG
GCATTGATGTACTTCAGGGGCACCAAAGAGAGAGTAATAGTGATCCAGGGTTTGGGAAATGGTACTGAACAGGGAAAATCGGAGGAA
ACCAAGGCAGAGCCATCTCTTTTGGAGTTATGCCTGGGCCTTCCAGAAGGAAGATGAGTTTGGTACAAATACTACATAATCACTGCA
CTGAGCTCTTGATGGAGCTACCAAGTAGGTGAACCTGGCAATTGCACATGTCCTTTGACCTGGAAGGCAGTAACAATGTGGTTAAGA
GTTGGGGCTTGGTGGTCCAATGGCGCTGGGTCTGGTCTGGAGGTAGCTCTGTCTCTGAATGCTTTGCTTCTACTGTGGGACTTCTCT
GGAGATACTTTCTTCATAGCATTGGTCAGAAGAAATGATGTCTTGTGGTGATCAAGTCCCATTACTGCCACATAAAAGATGCTAAAA
ATGACTTTAAGATGGCAGTTCCATTCTCTTCCTTTCACCTCTTTGTGCAAGATGGAAAACGCTATTCCCTCCAACTCCTGCATAAAG
CCGCCATCTTGTCCTTTCACAGATTTCCACCATACCATCTTTCCCACTTCTGGGGAAAGCGGCTTGATGATTCACAGTGTCCACATG
CACTTTGAAATCTTTATAGCCCCAATAGTCATGTTTATTGGCAGGAAGAGAGATTTGTTGCCCCAATACAAGCACGCAACTTTGAAC
CCTGGAACCAAGACTTGATTCTCATTTTCTGATGGGTGAGCCAGCTTGTCCTTGTTGGAATAGTGCAGCCTTGTTTGTGTGTGGCTA
ATGTAGTTTTCACTGGTGTGACTAACTGAAAGACTGCTTGCTGACATGAGTGCAAAGCTGATAAGCAGCAGCTCCACAGAAAGCACT
GACCTCACTTAAGCAACCATGGTGGTACCTGGACATCTGTCCTGCACACCCAGTTCTTATGCCCTGCCTTCTAGAGGAGCTGATGGA
ACAGGAAAGATGTGGAAAAAGGAGTGAAAGGGATGAAGACCTCCCAAAGGAAGATAAACTTAGAAAGGAGGAGATGGGCTAGAAGTC
TGGCAACATTACTTTGTGAGTTAAGGATGCATTTTCTTGGCCGGGCATGGTTGCTCATGCCTGTAATCCCAGCACTTTGGGAGGCTG
AGGCGGGTGGATCACCTGAGGTCAGGAGTTCGAGACCAGCCTGTCCAACATGGTGAAACCCCATTTCTACTAAAAATATAAAAAATT
AGTCGGGCATGGTGGGGGGGTGCCTGTAATGCCAGCTATTCAGGAGGCTGAGGCAGGCGAATCGCTTGAACCTGGGAGGCAGATGTT
TCTTGCAAGTAACAGGAAATTTGACTTATTTGACTTTAGAGCATTTTAAATAAATAGGGGTTTATTTTCCTCAGATAACAAGAAGTC
TGAAGTAGGCAATCCAGGGCAGGTTTAGCAATTCCATGATGTTGCAGAGACTTGGGCTACTTTTCTTTGCTACACCATTTTTTGAGC
TGGTTTGTCCTCTCAGGGTTCATCACAGCTGCCCCAACTGCAGACACAAGGTACATTGTCTCAGCAAGAGTCACAGGAAAGGGCAAT
GGCAGCCACAACTACCCTTTTTTTTAAAATCAGGAATATGGAAGCTTGACCACTGCCAGTCGACTTCCCCTCTCATTTCATGAACCA
GAACTGGGCCACTTGGCCACCTTCCTAGCTGCAAGTGAGCCTGGGAAAGGGAACTTGGTTTACCGGTCTCTGTGGTGGGAGGAAGAA
AAGGAGAGGGCTTGGCAGTGGCCCAGGGGCAGCAAATCTAAGGCCCTACCACAAATCTGAGAACAGAAGACCAAACTTCAAAGCCCC
TGTGTTCTCATTCATTATTGCTTTTTTGCCAGACATCCAGCATCCAACTTAGTCAAGGTTCCCCAGACTGAGCGATCTAGCATAAGA
CCTTTCTCTGATCACTGGCATATTGATGGCTTTTGGGTATTCCCAGCAGGGAGCAGTTTCCATGGCAAGAGTGTGCTTGCTCTCCCT
AAATAACTGGAAAATAAATGGTTTTCCCCCAACCTAGTGGCACCTGCACATCTGTCCTGCATACCCAGTTCAGGGAGTGAGAAGACT
TTTTACTCTTTTATTTTTATTGTCTAGCTCCAAAAACCATTGGCTTGCCACATCATGCTTCTGATTTCAATCTCTTTCACCCCTGAT
TTCCACAAATGGGAGACCCAAAGTCGTGCCCAGGAAGGAGAGAAGGCCAGCGTGGGGATGAGGCACTAAATCCCTGGTCCTAGAGGG
TGCCCCAACGTGGCTGCATGACTGGCTGTCCCTTCCCGTGGGTGCCCACTCCATCTGCCTTCAGAGGTGCTGTTGTGGTGAGGGCTA
CAGTGATGCTCCCCATGTTCTGAGACTGGGAATGCTGGGAGTCACACTCTCATGCACATCTTCAGTGCAGCCTGCCAGTTTTCCCTC
CAGGTTGCCTTGGCTGTTTCACCCGCTCTGGTGGGTGCTGGTGCATAGAGGCTGAGAGGGCTGGTTCCAGAGTTGCAGGTTCAAATC
TTGCTTCCAGCCACTACCTGGGCAAATAATTTAACCTCTTTGGGTCTTAGTGTCATCACCTGTTAAACAAGGGTAACAATAGTAACT
TTCTGCCAGAGTTGTTGTCAGGATTAAATGTGCTTAGGACAGAGCTCCACATGTACTAACTTAGCAAGTGTGAGCCACTCTTCTTAC
CATCCCAGCCACATTCACTACTACTCCAGTCTTGACTTGGGTGTCTGCAGAGCCTGCTTAGCTTTCAAGTTTAGGGTCTTTGGATTT
TGGAACCATTGTGGGGCTACTGCTTAAAGTAAACATTTTGTACTTTTATTTTTTTCTAAATGAATGTAAAGAGCTAATCAAATGTTA
AAATAGTAAGCTTTGCTTAAAGTTATAGTTCAAATTCAGATTCAAATTTGGCTAAAACTTACCGTGTAACTGTGCCAAGCAAACTTT
GTACTAAATACGTCTATATTTTCTTTCTCCCCCTTATAAATTCAAAACACTAGAGTGCTGGCAATCTGCTGTAAATGTCTTTATAAA
ATAAAAAGAGTGGAGGGAGGTGTTTTATGAAGCTGGAGTAAAAACATATATAATTTAGAAGACAGTGTGGGATTGTGGAAGACCATG
GGCTTTAGTTCAGTTAGGCCTGAGCATGTGTGCTGTGTTAACCTTTTTAAGCTTCATTTGTAATATGGGGATGTTAACATTCTTCTC
ATAGAGTGATTCTTCTCATCTCACATTCTGCTCAGATGTTTAAGAAAAGCAATGGAAAGGTTTGTAACAAGCTACTTTGACGTGGTA
GAGCTTCAACAGCTATTACACTCTTTCCTGTTTTGATCAGATTTCAGCCATGATAGAATCCTATGTTTTGTATATTGGTGCTGGAAG
CCCTAATAAAATCAATGAGTGACACAGACACTTCATACAAGATGCTAATTTTCTCTTTCTTTTTTAACAGGCAACTGGATATGGCCA
AGAGGGGAAGTTTAGTGGACCCCTGAAACCCATGACATTTTCTATTTATGAAGGCCAAGAACCGAGTCAAATTATATTCCAGGTAAA
AGCTACCCTTGATCACTGTCCTCTTGATTTTGTATGCTCTCTTGGTGGACTTTTACTTCCTTATCTATTGTATAGCAGGAAATAAAA
TAAAGGCATGGCATTCTCATTATCAAGAATGATTTCAGAAGCTAAGTTAAACAGAGAGATGAGTGGCCTCAGTATCATAAGAATTGT
ACCTCACATTCTATTTGGACTTCCTCAATCATCTCTGGGGCGAGTGCTCCCAGCCCTCTGCAGGGAGCTTGGTGGCTGCAGGCTCTG
CCTCTCCCCTAGAGTAGAAAGGGAACCTGTCTCTGGAGAGGGGAATGACCATCCACCCTCGAGGCTGTCTGATCAATGAGCTTGCTG
CTTCAAGTACTGGAAGATACGGCTTCTCTGGAGTCAGTGCTAGAATGAGCTTTCTGGCCAGAGGACTGCCTTTTCCTTACCACCTTC
TCTGAGCCCCCATGTGGGCCATTGTCAAGGTGCACTGGGAGGTGGACGTTTTATTTAGTTAGATAACCATGAACAGTATCAGATGGA
GGGGCAGTCTGCAGGTGGGAATAAGGGAAGGGACCTAAGTCCCATCTTAAGTGCTATTAAAGACACAGAAATAATCCTGGAAATGTA
GTGGAGAAGGGGAAAACACTACTGTATTTACCAGACATCCCCTGAGAAAGTCACTTGGCCCTGAAGTGATTGGAGGATGCTGATGAG
CCTTCTTGGGGGTGCCCGTGACCCCTCCCCGTCCCCTGAACCAGGGAAGTGGCAGAGGTTGCTGAGCTACCTACCTTCTAAGGCAAG
TGCCTTCCATTAACTGCCAAAAATTTACAGACGGCCCTCTGTATCTGTGGGTTCATGCATCAGCAAATTCAACCAACAAAGAATCAA
ATATATTTGGGAAAAACAATAAAAATGATACAAATTTAAAAATACAGTATAATAATCATTTACATAGCATTTACATGGTATTAGGTA
TTATAAATAATCTAGAGATGGTTTAATGTATATGGGAGAATGCGAGTAGGTTCCATGACCATAATACACCACTTTATATAAGGGCCT
TCAGTATCTGTGGATTTTGGTATCTGCGTGGGTCCAGGAATGAGCCCCCACCAATGCCAAGGGACAACTGTATTGTGCTGGACAGTT
CCCCTCCACAGAGGAGAGGCTGAAAGCTATCTCTCTTCCCCTTTCTTTCATTGGTACCAGCTTAAGGCAAAGGGCCCCAGAGTAAGA
GCTGGCAGGGGCATGCTTGCATGTCTATGCAGCCCGGTGAGGTAGGGTGTTATTAGGGGCCTCCACCTCTCCAGTCAGGGAAGTCAG
GGCCTGAAGAAGCAGTGAATGTGTTGAGAGCTGGAGGTTGATGGGAATTAACTTGGTGAGGAAGGGAGGGAAGAAAGCTTAGAAGAG
GCCCGGCAGGGATGGCTGCCCTCTGCCAGGAGAGGGCAGGAGACATACCAAGGACTAGCAGAAGGCCATGCAGGTAGGGAGGATGGA
GCCAGGAGGAGCAGGTGGGAGAGGAGGCCACAGAGACAGGGAGGACCCTGACTCCTAAGGATCTTGAGTTTTTCTCAAAAGCAATGG
ATCTGAATCAAGACTCCATTGAGAAAAGGTCACTCTGGCTACAAACTAAAGGAGGAAAGAGAGGATTCAGGGAACTGGCTAGGGGAC
TCTGGCAGGGGCTCACACAGAGTTGAATTACTTACTTTTTCTCACAGACTTTCTTTTATTAACCATGTACGGAGTGACTACTATATA
TACCAGCTGCTCTGCTGGGTACTGGGGATATGGGATGACCCCGACTCCATCCCTGCTCCGGTGAGCTTACCAGCTAGTAAGAGAAAA
CTAACAAGGAAACAGACACATGCACTGAAGCTTCTCATGGGAGGCTGGTGGGATGTGGAGGGGGCAGAGGGTAAACTAAGAAAGAGT
GACTGATTTTTATTTGTTTATGTTTATTTTTAATTGACAAAATTGCACATATTGTGTACAATGTGTTTTGAAGTATACAGAGAAATG
ACTAAATGAAGCTAATTAACATATGCATTATCTCACTTATCTCTTTTTTGTGGTGAGAACACTTAAAATCTAAGAATGATCAATTTC
AAATAAAGATGGTAGTGAGTGAACAGAAGAGGTTTCATTGACTCCTAAACTGAGTACTCAAAAACGAGCAGGTGCTCACAGTCAGGA
AGCAGGTGCTGAGTACAGGATGGGAAAAAGCATGGGGGTGTGCACTTGTCAAGAAAATGACAGCTGGCATGTGGGCCAGAGTATAGA
GTGGGAGACAGGATGCAGTCAAAGGTCTGCATGCCTGCGGAGGTCAGCACGGGCTGGACTAGCCCTTCGTCCTATAGCAGGCTGCAG
GGTGCTGCTGGAGGGTTTCAACAAGGAAAACCGTAACTAGATTTGTGCTGTAGAAGCTTACTGTGTGATCACTCAGAGCATGAGCTG
CAAGGGAGCGAGCCTAGACGTTAAAAGGATTTTAGAAAACTGTTTTCAATAGTTCAACGGGAAGTGAGGGAGGCCTGGTTTGAGTAA
CTATAGAGGTCTGAATGTTGGTGTCCCCACAAAGTTCATATGTTGAAGCCTAATCTCCAATGTAATAATATCAAGAGTTAGGGTCTT
TGGTGGGTGATTAGATCATGAGCGCTCTACCTCTATGAATAGGATTAGTGCCCTTATAAAAGACGCTTGGGGGTGCCTGTTTGCCTC
TTCTGCCATGTAAGGACACATGAAAGGTGCAGTCTGTGAGGAGTGGGCCCTCACCAGACACTGAATCTGCTGGTTGGATTTCTCAGC
CTTCAGAACTATGAGCGATAAACTTCTGTTGTTTATGTTTCTGAATGAGCACAGGGGATGAGGGAACACAAAGAAGGACAACTCTTA
GGTCTCTGGCTTGCTTGACTGGGTAGTTAGTGGTGTCACTGAGTGAGAGAGAGAGAGAGAGTACAGCAAAAGATTTAGAGAAAAGTG
AATGAGTTTTAGGCTTATTGATTTTAAGGTTATCTGTGGGGCAGCCAGGTGGAGAGGTCTCAGACAGTGTTAATATGGGTCAGGAGA
TACAGATTGGGGAGACTTCCTTATATAGGTGCCAATAGAGTGCTTGGATATGACATAGATACCTGTCTGTGGTGAGTTATCTCAACT
GCTCTTAGGGGCTAGCTGAGTGATAAATGAAGCCATATGGAAAAAAGAAAAACAACAGCCAAACAACAACAACAAAAAACAGATATG
AGAAAAAAACCCAACTGCACAAATATGAAATCAAACGCTTTCTCATACTTGTCTTTAGTATTTGGGACAAGAGGGAGTAGTGGGGAC
TGTGGCAAACTGAAAGTAAGTCAAAAGGGGATAGGCTTAATTTAGCTCCAGACGATTTTCACGAGTTAGAATTTGGGTACTACTCTA
CTAGATTTTATAATTTTCCTAGAAAAGGCATAAAATGTTATATTTATATAAAATTCACTGATGTAAAAAAATTGGCATAGTTTTTTT
TTAATTACAAGCTAAATAAAACATATTTGTGAAACAAAGTTTGTGGCTACTTCTGAGATTTCTGTATGAAAAGACCCAAGGTCAAAA
TAGAGTAGGTGTTCTGTGAGTACAGGTCCACACTCCATTGAAGACAATCAGGATTGTACATAGATGCAATCTTCAACTTACAAGCTT
TACAACGGACAAACATCCAGACCTAGAAACTGTTCAGTAGGATCATTCCAGAGTCTCTGGGGATCCTAGCCAGTAAAAGTCATGCCT
TGGGCTGCTCCACTTTTCTTTTTTTGTTTTGTTTTAGTTTTTCTGTTAGTCATGCTGCAAAGTTATCCCTTCTTAGCAGGAAATGAT
AGCCTGGTTATCATGGTTGAATGTTTCTTCTTTACAGGGGTGTGTGTGTGTGTGTGTGTGTGTGTGTGTGTGTGTGTATTGGGGTAG
GGTGTGGGGTTGGGATGACAGAGTAGTGAGAGGAAGATGAAGGGGATTTTCTTTTTAGAATCACAGAAGCTTCATGCTAGAAAACAC
ACTGGGGAGTTTTGGTACAACCGCTTGTTTTAAAAATGGGGAAACTGAGGCTTAAGTTAGTTAATGGCAGAGCCAACAATAGAACAC
AGGTCTCCTGACTTCTAGTCAAGCACATTTTGCTCTCTCCATCTTTGTACGTGGAAAGAATGCAGCAAACCCAGCCTTGGTAAGATG
TGCTCGGAGCTCTTTGCTTCAGTGGTGGGGAGGAGGTAGGTGTACAGATCCTCTCATCCAGCACCTGGACCTACAGAGGCATGAAGA
ACACTCATTGCCCCATGAGGTTGGTTCTTGGGTTTTTAATATGTCCTCAAAAATAAAATGAACTGAACCACCATACACTTCTTTCAT
TGTGGATTTTCAACTAGTGCCAGCTCTGTACTCACCAGGGGCTTCCATATCATCAGACAGTGTGATATAAATCTGAGAGAGGTCTTT
TTTTTTCTCATTTTAAATAAGATGTAACCCTAAAAATTCATGCCTCTAAAATTCAGTAGTTTTTAGTATCTAAAAAGACGTGCAAAT
ATCACTGCTATCTAATTACAGAACATTTCCATCAGCCTCCAGAGAAATCCTATGCCTATTAATAGTCACTCTCCAGCCTCCACTCCC
CTCAGCCTTGGGCAACCACTAATCTACTCTTGATCTCTATAGATTTGCCTGTTCTAGACATTTCATGTAAATGGAATCATACAACAT
ATGGCCTTTTATGTATGACTTCTTTTATGTACCATAGTATTTTAAACATTCATCCATGTTGGTAACATGTACCAGAACTCCATTCCT
TTTTATGACTGAGTGATATTCCATCATCTGGATATATCACATTTTGGATATCCATGTGTCAATTGATGGACATTTTTTTTCATTTAT
GGCTATAATGAATAATGCTGCTATGGCTATTCATGTACAACTTTTTATGAGGACATGTTTTTGATTCTCTTGGACACACACACACAC
AAACACACACACACACTCAGTAGCAGGACTGCTGGGTCAAAGTTAATTCTGTTTAAACTTTTGAGAAACCACAAACTGTTCTCCAAA
GCAGCTGAACCATTTTACATTTTCACCAGTAATGTATGAGTTCCAATTTCTCCATATCCTTAGCACTTGTTATTATCTGTCTTTTTT
TTTTTTTTAATTATAGCTATCCTAGTGGTTGTGAAGTGGTATTTCTTTGTGGTTTTGGTTTATATTTCCCTAATGACTGATGTTGAG
TATTTTTTCATGGGGCAATTGACTATTTATATATCTTCTTGGGAGAGATGTCTATTCAAATCCTTTGCCTACTTTTAATCAGGTTAT
TTGTCTTTTTATTGTTGTAAGAATTATTTACATATTCCGTATACAAGTTCTCTCCCTTATCAGATATGATTTGCAGCAAGTATTTTC
TCACATTCTATAAGTTATCTTTTCACTTTCTTGGTGGTGTCCTTTGGAGCACAAATGCTTTGAATTTCGATGAAGTCCAATTTATAT
TGTTATTGTTATTTGTACTTTTGATGTCATATCTAAGAAACCATTTCCTCATTCGGGGTCATAAAGATTTATGTCTATGCTTTCTTC
TCAGAGTTTTATAGTTTTAGTTTTTCTATTTAGGTCTTTGATCCATTTTGAGTTAGTTTTGTGTATGGTATGGGGTAAGGGTCCAAC
TTCATTCTTTTGCATGTGGATATCCAGTTGTTCTAGCACCATTTATTGAAAACACCATTCTTTCCCCCACTGAATTTAGCATCCTCA
TCAGAAATCAATTGAATTGTTTCTGGCCTCCCACTATGATTCCATTGATCTATATATGTTTATGCCAGTACTGCATTATCTGGAACT
ACACCTTTGTAGTAAATTTTGAAATTAGAGAATGTTAAGTCTTCCTACTTTGTTGTTGGCTATTCTGAGTCCCCTGTAGTTCCATAT
GAATTTTAGGATCAGCCTGGAAATTCTACAAACAAGGCAGCTGTAATTTTGATAGGGATTGTATTGAATCAGTAGATAAACTTGGGA
AGTATTGCCATCTTAATAGCATTAGATTTTTAAATCTGTAAACACTAGGTGTCTTTCCATTTATTTAGAACTTGTTTAATTTCTTTC
AACAGTGTTTTGTAGTTTCCATTTTATAAGCTTCATATTTCTTTGCTTAAGTTTATTCTGAGATATGTTATTCTTTTTGTTGTGGTT
GTAAATGGAATTTTAAAAATATCATTTTTGGGTTGCTATTTGCTAATGGTTAGAAAGATTGATTTTTGTTTATTGATCTTTTATCCT
GAAACCTTATAATACTTGTTTATTAGCTGTAATAGTTTTTTTAGGTTCCTTAGAATTTTCCCTATATAAAATCATGGCAACTGTGAA
TAGTGATAGTTTTATTTTTTTATTTCCAATCTTTATGCCTTTTATTTCTTGTTCTTGCCTAATTCCCCTTGCTAGAATCTCTAGTAC
AATGTTAAATAGAAGTGGCAAGATTGAGTATCCTTGTCTTGTTTCTGATCTTATGGGGAAAGCTTTCAGTCTTTCACCATTAAGTAC
AATGTTAGCTGTGGGGCTTACTTCGGTGTCCTTATTAGGCTGAGAAGTCACCTTCTATTCCTGGTGTGTTGAGTGTTTTTATCATGA
AAGGATGACGGATTTTGTCAAATGATTTGCTGCCTCTATTGAAATATGTGGTTTTTATCCTTGATTCTATCAATATGGGGTACTACC
TTAATTGACCTTCATAGATTGAACCAGCTTTGTATTCTGGTATAATTTCCACTTGGTAATGGTATATAATACTTTTCATATGTTGCT
GGATTCAGTTTGCTAGTATTTCATTGGAAAATTTTGTGCCTATATTCAAAAGAGACATTAGTCTCAAGTTTTCTTGCATTGACTTTT
TTCTGGTTTGGGTATCAGGATATTGGCCTCATAGAATGAATTGAATAAGTGTTCCTTACTCTTCTAGTTTTGGAAGAATTTCTGAAG
GTTTGATGTTAATTCTTCTTTACATGTTAGGTACAATTCACTAGTGAAGCCATCTGGTTCTGGGCTTTTTTGTGTGTGGAAATTTTT
TGATTACTAATACAATCCCTTGTTATAGTTCTATTCAGATTTTCTATGTGTTCTTGAATCAGTTTTAGTAGTTTTTGTCTGTCTAGA
AATTTGTCCATTTGATCTAGGTTGTCTAATTTGTGGGAAAACAATTGTTCACAATATTCTCTTTAATACTTTTTGTTTCTATAAGGT
TGGTAGTAGTCCTCTTATATTCCTGATTCTAGTTATTTGAATCTTACCTCATTTTTCTTGATCAGTCCAGCCAAAAGTTTGTCACTT
TTGTGGATCTTTTCAAATAATCAACTCTGGTTTTGTTGACTTTTCTCCTGTGCTCTTATATTCTCCATTTCATTAATTTCCACTCTG
ATCTTTGTTATTTTCTTCTGCTTGCTTTGCTATTCTTCTTTTTCTTGTTTTTCTTTTCTTTTTTTTTTCTAAGTAGAAGATTAGGTT
CTTTGGGATATTTCATCTTTGTAAAGTATGAACATTTACAGTGATAGATTTTCCTCTAAGTACTGCTTTATCTGCATCCCATAAGAT
TTGGCATGCTGTGTTTCATTTTCATTTACATCAAAGTATTTTCTAATTTCCCTTGATTTCTTCTTTGACCTTTGGTTTTTAGAGTAT
ATTGTTATTTTACATATTAGTGATTTTCCCAAATTTCCTTATATTGTTTTTAATTGTATTCAATTGTCAAATACTTTGTATAACTTC
AATCCCTTTAAATTTATTGAGACTCGCTTATGGTCTAACATATAACCTCTCCTGGAGAATGTTCCATGTGACTGGCTTTTGTTGGAT
GGAGTGTTGGGTGTTTGTTAGCTTCTAATTGGTTTATAGTATTGTTCAAGTCTTCTGTTTCCTTATTGATTTTCTATTCTATTATTT
AAAGTAAAGTCTTGAAATCGTCAATTATTACTGTTGAATTATTTAATTTCCTTTCCATTCTGTCAGCTTTTCTTTCATGTGTTTTGA
GGTCCATTGTTAGGTGCATATATGTTTATAATTGCTATGGTTTCTTGATGGATTGACCCTTATATCATTATAGAATATCCCATTTTG
TCTCTGAAAATTTTGTTTTAAAGTCTGTTTTGTCTGATAGTAGTATAGGTTAATTTTATCCTCATTTTGAAGGATAGTTTTGTAGGA
TTTACAGTTCTTACTTGGCCACTCTTTTTCTTTCAGCACTTTGTTTTTTTTTTTTTTTTTGGTTTGTTTGTTTGTTTGTTTGCTTGT
TTTTGTTCGAGACGGAGTCTCACTCTGGGAGTCTCACTCTGTCCTCCAGGCTGGAATGCAGTGGCACAATCTCAGCTCACTGCAAGC
TCCGCCTTCTGGGTTCACGCTATTCTCCTGCCTCAGCCTCCCAAGTAGCTGGGACTACAGATGCCCACCACCATGCCCGGCTAATTT
TTTGTGTTTTTAGTAGAGATGGGGTTTCACCGTGTTAGCCAGGAAGGTCTCGATCTCCTAATCTCGTGACTGCCCACCTCAGCCTCC
CAAAGTGCTGGGATTACAGGCATGAGCCACTGCACCCGGCCTCTTTTAGCACTTTGAATGTGTCATTCCACTGCCTTCTGATCTCCA
TGGAAAATTAGCTGTTAATTTTATTGAGGATCCTTTACATGTGATGAGTCTATGCACTTTTAAGATTCTCTATCTTTCAAATGTTTA
TGATGTGACTAAATTTGGATTTCTTTGAGTTTATTCTATTTGGAGTTCATAGTGATTGTTGGATATATAGATTCATGTTTTTCTTAA
ATTTGTGAAGTTTTGGGTCATTCTGTCTTCAAATAATCTTTTTTCCCCCTTTTTCCTTTCCTTCTGGAACTTTTATTATGTATGTGT
TTTTCTGCTTGATGGTGTTTCACTGGTCTCTGGGAATCTATTCATTTTTGTTCATTTTTTTTCTTTCAGTAATCTCTATTAATCTAT
TTTCAAGTTCACAAATTCTTTCTTTTGCTAGCTCAAATCTGCTTTTGAACACCCTCTTATGAATTTTTCCTTTCACTTATCATCCTT
GTCAACTGTAGAATTTCTATTTTAAAAATTTCTATTTTTAATCAATATTCTATATCTGATGAGGCATTGTTTTCAAACTTTAATTCT
TTAGAGATGATTTCCTTTAGTTTTTGAATCTATCTAAAATAGATTTTTAAAAATCTTTAGTAAATCCAACATCTGGGCTTCCTCAGG
GACAGTTTCTATTGACTTTTTAATATTTTTGTACATTTCATAATTTGTTGAAAACCAAACATTTTAAATAATGTGATGTGGCAACTC
TAGAAATCATATATCTTTCTTTTCCTGGAGTTGGTTGTTGATGCTGTTTGTTTGCTTAGTGACTTTCCATAATGAATTCTGTAAAGT
CTGTATTCTTTGTCATGTCTGGCCACTGAAGTCTGCTCAGTCAGCTTAGTGTTCAGCTAATGACTAGATAGAGATTTCCTTAAATGC
CTTAAGCCAGTAAGTCTCCTGGTGTTTACCAAGGGGCACTGTGTGCATGTTGGGACACAACCTTCAACTATGTGGCAGGCAGTTTAC
AACTCTGCCTTCGCCTTCACTTTCTGCTTATGCAGAGCCTCCAAGTCAGCCAGAGGTGAGGGATTAGGGCCTTCTCAGGTCTTTCCC
AGGCATGCACACAGCCCTGCTCATGATACTGGCCTTGTAGATTGCCAGGAATATATTAGAGATTTTCAAAGCCCCTTATGGACATCT
CATTTCCCAGCTTTCCCTTTTAAGTGTTTTGGTCAGCTGCTTCTTTGCTTCAACTGTTATCACAGCCTCAGGCAGCTGCAATGTTGA
ACAATTGCTAATGGTTGTTTTCAACAAATGCCCCAGGGGAAAAATTGTGTGTGGTGTATGAATTCTGAGTCCGGTCAAATGAAGATA
AGTGTTGTAAGTAGATGTTCCCAGGGAAATGCCAACAGGATAGATAATGATAGTTCTCTGGTGATGGAGCTTTTGGGAAGCTACAAA
CGCTTCTCACCCTCTCCATGGTACTGTGGTGTGAGGCTGTTGCTTTCAAGGCTACTTCAGATCTTAGGAAAGGACCATGGGATTAGG
GTGCATTAAAACACCACAAACCTTAGTTTTTTTTTTTTTTTTTTAATAAACATTCCTTGGATTTTGCAAGCATCTGGTTAATTTCCC
ATATTCTTAAAAAGATGATATTTACAATTTTTGCCAGTGTTCTCATTGCTTTTATGGAGGAGCAGTTTTCTGTAGGCCCTTACTCCA
CCTTTCTCACTGATGTCCAGGTCTTTTTCTGAAAACACAAAATAGCAACATGTTCATTTATTATATAAACAATATAATATCAACAAG
CAAACATGCACAAAAACTTTTTAACTTACAATCTTACCCATTTAACACATCAACTCTTCTTATATTTTAAATTCCATTTCTTACCTA
CTGACATGAATTCTTTTGGTACCATAACAGTCTAGATTTACTCTCAAATTTACTTCTGTTACTTTACATCATTGTTTCTTATGTTTC
TTCATAATTTGTAGAATGAACATTTTAATGATTAATATTCTGTCAAGTTCATGTGCCATAATAATTTCACTGTCCTTTTTTTTTGAC
CATTTAGGAGTTTTCAATCTTACTGGTATTATAAATAAAGTTACTTTCACTATAAGCATGTACTTGTTTGAAGAAATATTTCCTTAG
GGTAAATTATGTATAGTAATGTTTGCTATCAAAGGGTACAAACATTTTATGCCTCAAGTTATTTCCAAAAGGATTATAGGAATTTAT
ATTTCCATCAACAATGTTTCTTTATATCCATACTAACTTTGATATTGCTTTTAATTTAATAAAAATATATAAAAGTATACTAAAACT
TAACGTTGAACAGCTTTTATGTTTTCTTAGTTTTATGTGAATTTTCTGTTGCTGTGGTCTTAAAAGTGATATAGCCATATCTATGAA
TATGGTGGTCGGTATTTTCTCCACTTCTGAAAAAGTAATAGATTATCATATAGCATTTGCATTAAGTTAAAGCCACTTATAAATTCT
TTTCCTTAACATTAACGTGTCTATTAATCTTTGTCACAATTTTTAGAAGTGCTAAATGACATATTTTATCCTAGAGAGAAAGCTACC
CTTTTAATGCTTCTTAGAAGCTAGTAATGTCATAGATTTCAGATATTACCAAAAAGTCTCTTTTAACCCCAGCTATAATTTAAAGCA
TCAATGACTTTTGTCAATGTATTGAATAATAGTATACAGTCTCTTGAGGAAAATGTATGTTAAATGTTACACAGTTATTGTGCAAAC
AAACTTTTAGAAGGTACCACTTTAAAAGTTGAGGATTTCTTATTTAGTAATTGAACCAATGAAAATTTTACCTGAAATTGGTGACTT
TCTACAAATTCCACAACATTGTTTTTTCCCTTACAACTTAATCTGTTTTTTGCTTTTTCCCCAGTTTAAGGCCAATCCTCCTGCTGT
GACTTTTGAACTAACTGGGGAGACAGACAACATATTTGTGATAGAACGGGAGGGACTTCTGTATTACAACAGAGCCTTGGACAGGGA
AACAAGATCTACTCACAATCTCCAGGTAAATTGATACCAGCTGAAGGGATCTAACTCCAACCTCTCTGAGCACCCTTTGTTCTGGAA
GTATGGCTGGCACCTTCCTTCCTTGCACAGAATCCAGTTACAGCTTTCCTTACATTAATGGGACGCTTTGCAATCCTCACATGTCCA
TTTCTTACCAACTACAGAGGGGTGTGGCAAACATATCCTTTTCCTTAGGCTTTTTAACCAACGTAACCCCACTTACATTGCCAGGAC
CCTCTGATGGCATTACTTATCCAAGTAAGACCATGCTTAGGTACTTGGATCAAAAGTCAGCACATCTCTGATACTTCAATGGTATCT
GCCTGTGGCTGTTGCATTGGCTTGGGACCCATGAAGAAGGCTGTATCGGTGCTGGGATGGAGCTCCTTTGGGCGTTGGAAGAAGCAG
AGGGAACCAGTAGCCAGGAGCTGACTAAAAAGACCCGTCTCCCTCTCTTTCTCCTTTAGTTTTCACCGTTGGTATCTGAATACCTAG
GAAAGTCAGAGAGCCTACTATGGAAAAAAGAGTGAGATAAAAGCTTTCTGTCTGGTACTTCTGCTGCAGGCCTTGCAGGCTGACCGC
CATAATGGCTCTGGAAGAGAACACAACACTTTCATATTCTCAGGGGTCACAGTGCTCTTGTTTCATGGACTTCACCCATCCAGAACA
GATGCATGTTCAGTGTCCAGAACACCCTGCTGATAGAAAGTCACCATCATGATTAGCCAGGCTTAACTGATACAATCCCTGTTTTAG
CATTTCCCAATTTGGCCTTTTGTGAGTTCTTTCTCTCCTACAGTCACCAGGCCATTGGTTCTTTGAGTTCTAATTTCCTTCAAGTGA
TTTTTGGGCATGTGACATGAAGTTCAGTCTCACCAACATGGTTTCCTTTTCCTCAACATCAGGTTGCAGCCCTGGACGCTAATGGAA
TTATAGTGGAGGGTCCAGTCCCTATCACCATAGAAGTGAAGGACATCAACGACAATCGACCCACGTTTCTCCAGTCAAAGTACGAAG
GCTCAGTAAGGCAGAACTCTCGCCCAGGTAACAGGCAGGGGCCAGAGATATTTATGCAAAGATGGAAGGGTGTCAGGTGGAAGGGGC
AGACTTGTGTGGCTAATAGCTGCAGCTGTCACTCAATAAGCACTATCTTACCTACATTTCACACTGATTTGGGAGTTGTTTCACTAT
CTCTATTGCTCCAGAGCATGGTCTCTCAATGCAGCACTATGTATTGACATCTTGGGTCTGATAATTCTTTTTTTGGTCAGAGGCTAT
TCTGTGCCTTGTAGGATGATTGGCAGCATCCCCCTGACCTCTATCCACTATGTGCCCCCCTGCCAAGTTGTGGCAATAAAAATGTCT
CCAGGCATTGCCAAATATCCTCTGGTGGGTGGAGGGCACAAAATCCCAGTACTTTGGGAGGCTGAGGTGGGAGGATCACTTCAGCCA
GGAGTTTGAGAACAGCCTGGGAGTTTGAGACCAGCCTGGGCAACAAAGTGAGATCCTGTCTCTACAAAAAATAATAGTTTGCCAGGT
GTGGTGGTGCATGCCTGTAGTCCTAGCTACTTGGGAGGCTGAGGTGGCACAATCACTTGGTCCCAGGAGATTGAGGCTGCAGTAAGT
TGTGATCACACTATTGCACTCGGTCTGGGCAACAGAGTGAGATTCTGTCTCAAAAATTACCCCCAGTTGAAAAACGCTTTTCTAGGG
TGATCTCTAAGATACCTCAGAGCTCTAGAACACTCTAGAAAGGTAAGCTTACTCCCCTGTATCTTCTTAGGAACCTCCATTGATATT
GTCACTGGTGGTTTCCAGCTCTTTTCTTCTCTTACATCCCATCTGTGGCTCAGCCCTTCTCTATGCTGCATTGACCTTCTACCTTTC
ACTTCCACAAAAGCCCTTCATTAAGCCCTTCATTAAGGCCTTCATTAAGGCGTGAGTGTGTCCAGCAGGCACCTAGGAAGGAGCATC
ACCTCCTAACAGTTGCACACAACATAAATGAATATTAGACTTCAGAGTTCTTAGAAATATGGTTTATCTATTAACATTTACTGTATT
AGAAATTTTAAAGATATTCATTTAAAATAATACTAATCAAGACTTGGGGCCATTTGCATCATCCCACCATCAAGCTTCTTCCTTCTC
TTTCTTCAATGCCTGCCTCCGAGAATTCCAGGAAAAAATTCACTTCAGTTTATTATGTGGAGTGATGTCCCTGTCCCTGAAGCTGCA
TACCAAAGACTCACACAGACTCTGCTAAATGTGTAGGCTCCAGGCCGTACCTTTTCTCTCTTCACTAATAGAATCTGCTAGAGAAAT
GGCTCTCAAACTTTTTGGTTTCAGGACTTGTATATTCTCTTAAAATTTACTGAGGACTCCAGAGAACTTTTAGACATGTGGGTTATA
TCCAGTGGTGTCCTGGTAAAGGCTTAACAGCTATTTTTCTAGGGGGACAAAGCTCTGATTTGTAGAATTTTCTAGTTTCCATGTTGC
AAATACTCCTACCATGGCCTATTTCAAGTGACCATTGGTATTAATAATTGACACACAAAATTTCTGAAAATTCAACAACTGAGTCTC
ATGAGCTGTCTCTAGTACATCACTGGTTATATCTATCAACATTTACTATATTAGAAATTTTAAAAAATTATTAATTCATTTAAAATA
ACACTAATCAACATGTTTTAATGAAAAATAATTGATTTTAAAGACAAAAATAGTAAGAAAAATGGCATTGTGTTAATCTTTGCAAAT
CTCTTTAATGTCTGGATTAATAGAAAACGCTTGGATTCTCATGTCTGTTTCTACATTCATTCTCTTGCCATGTGCTAGTTTGGTTGA
AATATATGAAGGAAAATTAGCCTCACACAGATATGCAGTTGGACAAGAGAGGAATATTTTAATAGTATTTTCAGATAACTGGACATT
CTTTAGTACTATACCCAAACTCAATAAATTGTGGTTTCTTAAAGATTAGTTGCAAAGTGAAATCAAAATTAATACCATATTTTCCAT
TCTTTTACACTACAATTCATTAAATTATCTTGTACTTTAAATGGATCTTTTACAGAAACATGACCTTTAGCTTTACAGAGCACTTGA
ACAGTTTCAGGGACCCTTAGAGGTTCTTGGACCAGACCTTGAGAAACCTTATTTTAGGGAAGGGGGGAGTGGGATAAATATTTATTT
CCCCACTGGTGTGAATTGTCATACCTTTCCCTATTATATCTCCACTTTTTAGATGGAAAAAGTAGCTATGGTTGGTTTGGGTTTCAA
TTAATATCTTTTTTTTTTTTTTTCTGGGTACGTCTTGTTAAACAGGAAAGCCCTTCTTGTATGTCAATGCCACAGACCTGGATGATC
CGGCCACTCCCAATGGCCAGCTTTATTACCAGATTGTCATCCAGCTTCCCTGATCAACAATGTCATGTACTTTCAGATCAACAACAA
AAACGGGAGCCATCTCTCTTACCCGAGAGGGTAAGTTCAGGGGTGCCCTGGTAGGACTGTCTCGGTCAGAAAAAGGGAGTTTGGTCT
GTCTCTTCCAGAGACACTTCCTATTCCCTTCTCCCCTACTTCTCACAGGATCTCAGGAATTGAATCCTGCTAAGAATCCTTCCTATA
ATCTGGTGATCTCAGTGAAGGACATGGGAGGCCAGAGTGAGAATTCCTTCAGTGATACCACATCTGTGGATATCATAGTGACAGAGA
ATATTTGGAAAGCACCAAAACCTGTGGAGATGGTGGAAAACTCAACTGATCCTCACCCCATCAAAATCACTCAGGTAGTACCCAAGC
CCAAGTAACACTGAGAGCCAACTAGATAAATGGCTTCACTTTGTAGAGACCCACCCTCACAGTCTTCACTGATACACTTATTAGATA
CCTTTTTCATAGCATGATTATAAAGCAAATTATATAGCCTCTGGCCCCGAGTCACTGGACTTGACCATGCATCATGGGCAGGCAACT
TGGATGCAGATATTATGTTAGGCCGTTTTTGTATTGCTATAAAGAAATACCTGAGGTGGGGAATTTGTAAAGAAAAGTTTAATTGGT
GCATCGTTTTGCAGGCTGTTCAAGCGTGGCTCCAGCATCTGCTTCTGTTGAGGGCCTTGGGAAAATTACGATCATGGCAGAAGGCAA
AGGGGGAGCAGGAGCATCATATGACGACAATGGGAGCAAGAGAGTTAGGTGGGAGGTGCCACACACATTTAAGCACCAGGTCTCATG
TGAACTCAGAGTGAGAACTCACTTACCACCAAGGGCGTGGTGCTCAGCCATTCACGAGGGATCTGCCCCCATGATCCCACCTCCCAC
TGGGCCTCACCTCCAATACTGAGGATTACGTTTCAACATGAGATTTGGAGGGGACAAACATCCAAACGACATCAGATATATACTTTT
GCTTTCAGAGAGAACTTCACTCACCCTCACCTTAAAAGTTGACAGCAGTGATGTTTGTTGTTCCCTGTTAGCAGTGTGGGATATGGC
AGCCCTGCAGTGGGTCATTTATCGTGCACTATGTATGGACATGGTTTGATTTGGGGGTTGCAAATAGATTTGAAAGGCTGCTGCTTC
TTATTGTGGAAAAAATCTTGACCCAATAGACTTGCCCATCTTAGGCTGAGGGACGGAGATAGGACTGGCTAGGGGTAAAAGGATCAT
AGTAGGGAACCTTTCCCCTTGGAAACTCCCTCACCCCTGGCACTCTATCTCCTGTGTGTGCCTCTCCCCTCCGTTCAGATGCTCCTA
CTTCCCCACCTCTGTGGCATGTGCTCTTACCCTGGGCCCCCTCCTACCTTAGCTCCTAGAAATTCACTCTGGACTCCACCCAGATTT
GATATTTTCTGGAAGCCCAGGAAATTCCTCCACATGAAAGCTGTGGGTTAGTGGGAGATCCCTGGATTTGGAAGCAGATGAACAAAA
TCATATTCCTGTTCCATCACTTGAATGACTGATCATAAACAAGTCATTTAACTATTCTGAGCCCCAATGTCTCATCAATAAAATAGG
GATAATACTTCAGTGTTTTGTGAAGATTAACTGAGACAGTATCTGTAAAAGTAGATAAAAATATTAGATATAAAAGCTATTATTATG
AAGAAGTACTAGTCTGTAGTTCCCCTAATTTTTTTTTAATGCTACTATTGTTAAGGGGCCCTGGAATGGCCTATTCCATCATATTTA
TTGGGATGGGCCCAGGGACATTTATTTATTGAATGTTGAATTAATGAACTTCAACTTCAGGAGGGGAAAAAAAGGAAGAACAGCAGG
AGAATTCTGATTTCCCAGTTAGAAAGAAAGCATGTCAGCACTCATTACCATGAGAATTTCTGATTGGGGGCCTAGACTAAAGTCTTT
CATAGCTATTATTCAGGTTACTCCCAAAGGGATTGGTAAATGCTGCAAAGAACTCATGCTTAGTTGCCTTGGGGCTGCCAAGGGAAA
ATTCTAGAGTGCCTACCCAATTTTCAGGTCTGGAAAAAATGCTCTGGGTGTTCTGAAACCCAAAAGGAGAGAGGGAGAAAGAGAGAG
GGGGAGAGGGGGAAGGAGGAGCGGGAGAAGGGGAGGGAGGAGGGGGAGGAGGGGAGGGGGAGATGGGAAGAGGAACTCAGACTGAAT
TTTTAGAACACAAGAGGAGCCCACTTTAGAACCAAGAAGGTAGTCTGCAGGGACAATGGGTGATGCTTAAATAATTAGCAAGGGACA
AAGTTGCTGCTGTGATATCTTCCCAGGCTGCTCTGAGAATAGTTTACTGTGGAAATTTCAACTAGCAGTAGGACCTTGGGCAAGTCG
CTTAAATTTTTTGTGCCCTAGTTTCTTCATTATAAAATAGAGATAACAGTAGTCTGTACGTCAGAGGACTGTTTGGAGATTAAATGA
GATATAACACATAAATATGTTTAGAATTGTGTCTGGCATGTGGCAAACTCAGTAAACATTATTAATATATCAGACCAGGCTCTGTCT
ATATAGCAAGTAGACTGGTCTTACTTGTGTTTATTTTTTATTTTTGCCAGGTTGTTTACAAACAACCCTAGGAGTTGGCAGTTATTC
TCATCCCTGAAGTCTCTCAAAGGCTAATATTTAGGAAGGAATGTCCTTATTTTTGTATCATTTGTCTTACTTAATGGCATGGAGAAG
ACTTCAGAGTGGCCCCTATTGCCCCTGTTCATCTTCATTGGTGAGTATTAGATGCTCACCATACATTGTGGCTTAGTCCTCAGACAC
TGCCAGCCACAAAGTACATTTTGTTTCTTAAATGGATGGTTCGTGGATAGAAAGCTTTCACAGTCAGACTTCCAAAAGAAAGCAGCC
CAGAAACCCTGTAGAGGGTTTGTGAAGAATTGCTTTAGAGAAGAAACATTTCCTGGTGTGCTTTTTTCGAGCAATATAACTTGGTGG
GGGGAAAAAGGCTTATTTTAGGTTAAAAGTGAAAAACGTAATTCAGTTGTTACAAGAAACCACAAACATACAAGGTTGTCAGAGATT
TCAAATTGAGAACTAGAAAATTATTTCCTTTCCAGTTCTCTCAGTCCAAATACAGCTTTCCTCACAACTGACTTTGGGCCCTGTAGG
TCCGGTGGAATGATCCCGGTGCACAATATTCCTTAGTTGACAAAGAGAAGCTGCCAAGATTCCCATTTTCAATTGACCAGGAAGGAG
ATATTTACGTGACTCAGCCCTTGGACCGAGAAGAAAAGGATGCAGTGAGTAAAAGAGAAGAGTTTCACAGGCGACAAGACAAAGTCC
TATGCCCATCTACAGTTGAGACCAAAATGGATTTTACTTTAAAGAAAAAAAAGACACACATTTCAGACTCCAGCATATTATTGGTTT
TCAAAGACATGATAATTTTCACATAATTCTGACCCATCTCAGTTTTTAAAATTTTCCAATTCTAAGTGAATTAAAAATAAGGGAGTA
AATGACATAGAAATGAAACAACGATTTTCTCTGCTTTTGACAGAGGGTGGTGGGTGAGTCTTGCCTTAACTCTGACTTTCCTGTTGT
AGTATGTTTTTTATGCAGTTGCAAAGGATGAGTACGGAAAACCACTTTCATATCCCCTGGAAATTCATGTAAAAGTTAAAGATATTA
ATGATAATCCACCTACATGTCCGTCACCAGTAACCGTATTTGAGGTCCAGGAGAATGAACGACTGGGTAAGAAATGAGAGCTGCCTA
ATGCTGTGTAACTGCCCTCCATTTCTCTCCTCTGCTGGGTAAAAGGTGAGCAGGAGACCTTACTGGGAGTCAGTAATCCATGTCTTC
CACAGCATAGAGGTTGACTGTACCTTGGGAGTCACGTAAGGACCGTATTTGGGATCAAACAGTATCTGGATAGTATTCTTAAATTGG
GGCTTTTATAGCTACCATTACCGCTACTACCATTATCACTATCATTATCAACCTTACTACTATCATTAGTATCACAGCCACCTACAC
TAGTATCTTATATACATGTATATAGGGATATCTCATTTGCAGGATTTCCCCATACGTTATTTGACTTCTAATAATCTCATTAAAGAG
ATAATCTCATCACATTTGCCAAAGAATTTAACATTTACACCCAAAGTCAGACAGACACCCTGCAATAAGTAAGCAAACACCAATGGA
AGTTGACTTTTGACCCTTGGGCCCCCCTTTCCAAGAAGCTGATAATATAATTCAAGAGGCTGCTAGATGAAGTCAAATCCCATCTAT
TTTTATCATTTTTGCTTTGTCTTGCTATGTTTTCTTTTACTGTGTCCTGTGATAGCTTTTTAGTTTTTGTATAGGAACAACTACTAT
AAGAACATTATAATTTCTGGGAGTTTTATGGCTTCTCTGGGTTTGGGACAATGCTTGTCTGTTTCCCCCTTGGCAGTGAGCCCCTTG
ACTGAGCAGACTGGCATTTCTTGAACTGTTTTGTTCTGTTGTGCTGTGTACCTTTCAGAGGCTTCCAAGTGTAGCAGCCTCACGACT
ATCATTTCTAGAGCTTCACTGCCCTTGTGGAGAGATCCTCCTGTGCTTTGTGGATGGGCTCACATTTTCTTTTCGTCCCTCCATTCC
TCACCAGACACACCTCTGACAGAGTGGCTGCCTTCCCGGAGGCTTGGTGGGCGCTGCTGGAGCGGCTTGCTCACACTGCTCATTATT
TTTCTTCTTGCTATCAGGTGTCGTGTGGGCTGCCTTTTGTCAGTGTGTTCTGTGTCACTGTGTTCTGTGCAGGTCCTCTCTGTTTCA
TTTTTCTCAACTCTTGCCACAACAGGAAGAGTTAATGCCAGCTTGAAGGACTTCAGTTCGTTATAAGGAAGAATCTTCTCACAGTCA
CAGTAGTGTAACAGTAGCCAACAAACACGTGTGCCATCACCTTCTTGGGAAATCTGGGATGGTCTGTGACACTGAATAAAGGTGGAA
AATCTTTGACAACTTTCTTGACCCGGACATGACACTTCAAGCCTCTTTTACATTGGGCCAATCATATAGTCTATTTGTTACCTATTT
CTGCAAAACAGATTGCTCTAAATCTTGGCAGCTTGAAACAACAAGCATGTATTATGTCACAGTTTCTGTGAGTCTGGAATCTGGGTG
AAGTTCAGCTGGATACCTCTAATTCAGAGAGTTCTGAGAGAGAGGGAAAGAGTACGGCACAGCAGGAAAGGACAGGCATCAGTCTTT
CTATGACCTAATCTTGGAAGCGATCCCATCACTTTTGCCATTAGAAACAAGTTGCTAGGTCCAGCCTGCACTCAACAGGATGGGGTT
CTGAAAAAGCACAAATATCAGGAGATGGGGGTTGCTGGAAGCTCTTTTCGAGGCGCCTACCTCATCTGCGAAGAGGATAGTGCTGGT
TTTCTGAGACTGAAGAAGTCATTGTTGGTAGAATAAATTTTGAAGGGGCTCAGCTTGCTATTTGCCTCTCTAAAGGTGAGAATTTAT
TTTGTAAAAATATTTCAGAGTCTTTAGTGGACCAAGAATTCTTATAAATCAACTGAGCATATTCTATATATGTAAGCTTAAAAGGGT
ACAAGACAATGCTCCTGCCTTGTGGGGACTTACTGTCTAGCTTAGCTGATAGGACATGAGTAGGTGGAAATATTCACAAATAAAACC
TTGATAAAATACTGAATTAGGCCAGGTGCAGTGGCTCATGCCTGTAATCCCACCCAGCACTTTGGGAGGCCAAGATGACCAGATCAC
TTGAGGCCAGGAGTTCGAGACCAGCCTGGCCAACATGGCGAAACCCTGTCTCTACTAAAAAAAAAAAAAAAACTGCAAAAATTACCC
AGGTGTGGTGGTGCATGCCTGTAATCTCACCTACTTGAGAGGCTTGTAGAGATTGCAGTGAGCTGAGATCATGACACTGTGCTCCAG
CCCAGACAACAGAGTGAGACTCCATCTCACACACACACACAAATATATATATATATATATATATATATATATATATATATATATATA
TATATATATATATTCCCCAGTGTATTTACATTCTCCTGTTTGGTATCTTGTGGAACTCGTGTCCCCAAGTCTTAGCAACACTAACAT
TTATTCTTGAATCCCACTTGCCACCAAGTCTTATGTATTCATACAGAAAATTTAGACTCTCATAAAAACCCCACCTCCCTAAATATT
TCTGCATATTTCTAAGAGATGCTACGTATACTCCTGTATCAGTTTGAGTTCCACTTGTTCCAGGCCTCAGTCACTCAGAGACATGGT
TTTCCTTTCACTTCCGACTGATCCACAAGGTCCTCTGCTCCCAGCGAGGGGTGTCCTCTGATGCCAGCAGTCCTCATGGTGCCTCTG
GCAGCATGGAGGTTTCCTGTGAGTGAGAGGAGGACAGTGTCCTCAGTTTCTTGCTCACTTGCTGAGGGGATAGGGCTATAGCAGGGC
TGCTGAGACACCAAAGGACCACAGAATTTAGTAAGACACTCTTTCATCCCAGCACACAGATACTTTTCTTGCAAGGGCATATGCATG
CCATATGCACTAGAAACATATTCTCTTCTGTCCTATTCATTCCGATGGTCCCTGCATGCAGCCCATGAGGCACATCTGCATGTGTCC
AGCTCCACTGTAGACATACATAGGTACTTTCTTTTTCCTCCCCCAACCCAAAACCAAAAAACCTCTAAATTTCCATATGCGTCAGTG
GATGCAAACTGTGTCAGCTGATGGGCGAGGGGGTGTAACTCCACAAATGGGATGAATTCAAGTTTGAGCTCCTACGTGGGATTCTTG
CTACATATAAAGAGACCTACAATTCCCACCTCCCAGAGTAGGATGGTCTGGAACCTGTCAGAACTTGCCCAGAGCTGGGTGCAGAAC
CGAGTCATAGCCACTGGTTTGTAGAACTCCCTGGACTGAAACCCTCACAGGAATAAAATACTTAGGACCACAAACTACAGCAAGGTG
GAGAGGATCCTGCCTGGTCCCTGCCCTGTGTTATCTTTGGAGTAGAGTCAGTTTCTGCAATTGTTCCATGAAGTTGCTTAGATGCAA
TCTAGGGTTGGCTTCTCTTTGTTTAGTGATACCATTTGCCATGTGTATTAGCGTCCTAGGGCTACAGCAACAAATCACCACCAATTT
GGTGACTTAAAACAACAGAAATCTTTTCTCTCCTAGACCTGGAGGCTGGAAGTCTGAAATCAAGGTGCTTGCAGGGCAGCGTTCCCT
CCAGAGTCTCAAGGGGAGAATCTTTCCTTGCCTCTTCCGGCTTCTGTTGGCATCTGGTGTTCCCTGGCTACTTGTGGCAGCCTATCT
CCAACTTCTGGCTTCCTCCTCCAGATCATTTCCTCCTCTGTGTGCATCTCTGTGTTTCCCTTCTTATAGGGGCACCTGTCACTGAAT
TTAGGGCTGCCTCTAAATCCAGGATGATTTCATCTCCAGAGCCTTAACTAGTGACATCTGTGAAGGTCTTATTTGCAAATGAGTCCC
ATGCTCAGGTTCTGGTAGACATGAAGTTTTGGGAGGGAGTACTAGGTGTTATGGGCTGAATGTGTCCCCGTGCCCCCCAAATTATAT
GTTGAGGTTGTAACCCTCTGTTCTAGGCTACATCTTCCCCCTCCCCCAGATTCATAGGGTGAAGTTCTAACCCCTAGTACCTCGGAA
TGTGACTATATTTGGAGATAAGGTTTTAAAAGAGGTAATGAAAGTAAAATGTCCTTACAGTGGGCCCTAATCCAGTATGACTGGTAT
CCTTATAAGAGGAAATGTGGACATAGGGAGGTTCAGAAGGAAGGTGCTGTGAAGGCACCGGAAGGAAAAGACAGCCACTCACAAGCC
CAGGAGAAGCCTCGGAAGAAACCAACCCCAGCAACACCTTGATTGCAGACTTCTGGCCTCCAGAACTGTGAAAAAATAAGTCTCTGT
TGTTTAATCCATGATACTTAATGACAGCCCTGGCTGACAAATACACTATTCAACCCATTACATGATAGGATGAGAAGAACCAAAATA
ACTCTAAGAGCATTTGTCTGCTATTCAAAGTTATTCATGGTGTTTTCTAGGAATGTGCTTATTTTAGTGAAACTATTTTAATAGATT
ATGTGGAGCCTGTAATGGTGGGTTTTCCTTCTTTCCTTCCTCATAGGTAACAGTATCGGGACCCTTACTGCACATGACAGGGATGAA
GAAAATACTGCCAACAGTTTTCTAAACTACAGGATTGTGGAGCAAACTCCCAAACTTCCCATGGATGGACTCTTCCTAATCCAAACC
TATGCTGGAATGTTACAGTTAGCTAAACAGTCCTTGAAGAAGCAAGATACTCCTCAGTACAACTTAACGATAGAGGTGTCTGACAAA
GGTTAGTATCATATCCACCGTCTTGAGTGCAAATCATTGTTTTCCTTCTAGCTATGAGTTATTTTCCTGCGCTAATGTCTGGTATGT
TATAGAATGATGCCATTTAAACATTCTTTGCTTCCTTTGTGAAACACACATAACACAGAGATAGCTGGTTGAACACTGTAAAGGCCG
GAGCTGCAGCTCAGGCACCATTGTTGTGTTGGTCTGGGTCCTATGGGAAGCAGATGCCAAAAGGAGATTAAATGTACGAGGATTTTA
TTGGGGGGAGCCCGTGAGAAAAAATTGCGGAAGGGCACAAAGAAGGCTGGGAGAGTCATCAGACCTAAATACAAGCCTGATGCCTGA
CGCTGGGTGAAGGGGAGATGGAGAAGGAAGGCTGGTGGGAATGTCCTAGACTAGGACAGTCTGAGGAAAGCTTGGCACAGCTGTCGG
GGAGTCTAAAGGGGGCTGTCAGAGGAATTCATTGTCTTTGAAAATGAGCTTTACAATTGCTGCTTTTCTCAGTCACTGGCTGTGTAC
AGTCCAGGGGAGTTGAGGTCTCAGTGTGAATGTGGTAATAGATTCCAGAGCACAGCAGTGGAGCTGTTGTCAATTAACAACCCTGTT
GTTGGAGGTCTGCCAGGCACATTCTGCAGAACATAATCACAAACAGGAAAATGAATTGTATTTCCCAATTCTTGCTTTCAGACTGAA
AGGACTCTACTGTAGGAAAGTGGAGGAGAAGAGCATCTTTCGGAAAGTTCTATCTTACCCTAACAGCTTCTAGTTGGCCGTTTGACA
GTATCAGGAGACATTCTTCCCCATACTCCTTATACAATTGCAGAGCAAGTGGTTCTTAATCTCTATTGGGTCACAGGAACCTTTGAG
AATCTGATAAAAACTATGCATTCTTAATCAACTGCACACATTGTTAAACAAATGTGCACAGACACACAAAATACCTGGATTCAAAGA
CAGACTCATTTCAAGGGCACTCAGACTCCTAGAAGGTCCTTTGAGTTAATGCGTACTAATCTTCGATTACCATATAATTCAGTAATT
GTTCTGCTGGCAGGCACGGATGACACAGGTATGGAGAACAAACAAGAAACGTTTCACACTGTGCTTCATGTTTCTGTCCTTTACAGG
GATATTGAGATACTAGGCAGAAGCCAGGTTTAGTAGGGCCCTAATGCGGAATGTTTGAAACCCAGTAATTTGACAAATATTAAGGCC
CTAATATTTGTCTAGCATTATTGTAAGAGATAGAACAGATGTAAGTATAATAAACCTTCCCTATTCATGACGAGCTAACAATTTCAC
ACTATCAACAGTCCATTCTTTCAGCAGTTATTCTGCATTTACCTATTTGAGGCACCATGCAAGGTACTGGGGAGACATAGATGATTC
TGTCCTCAAGGAGCACTCATTCTGGTAGAAGAGACGGACATGTGAGTCAGTAAATTGCAAGGAACTGCCCAAGGTAGCCCATGAAGG
AAATAAGTGACTCAGTGGAGGGAGAGAACAGGTACTGCTTTCTGGAAGAGGTATTTCTGAGCTGGGCTTTGAAGGATGAATATGACA
AGTTACAAAATCATGTAGGACTTATGGGAGAGTGGGAATCCATGTGGAAATACATGAGGACCTGGAGCAATTTGAAGATAGAGTTTA
AAGTGGGAATCAGATTACAAATTGTTTATTTGCATGAGTACGTCTATAGAAAATATGGACCAGATTATAAAATATATTAAAATACAT
GTGTGTGTTGATATAACTAAACATTCAACTGTGCACTACAGAAAACTAGAGACTAGGCTATAATTAACACAGTTTTACATAAAACCA
GGGAAGGCCTTTTACCCAGAAAGGCCCACTGCTGGTCAAACGAGGTGTGTGCAGAGCAGTGAGCCAGTGGGTTCTGGGTCTGGGACC
ACTCCAGTCTTCAGCCATTCTTGTTGTCTACAAACGTTCTGAGGACCAGCCTTAACTTCAATCAATATCCACTTGGTTGTAATTGGA
TTGTTAATAGATTCTGTCCCTGGGATCATGTCCAGTGCATTGAGCACCAGCTCTAGAGTTAAACTACCTGGGAATATTAGTCAGGAT
CCCAGCAGGAAGTGGAGACTCCCCTCAAAGAGTTGAAAGAAGGGATTGTTTGCAAAGGTTGTGGGCAGGGTTAAGGGAAACCAACAG
GAGGTGTGAAGCACAGTAAGCAGCATTTTCTGCCCTAAAACCTGAAGGAGCCAGGGAGGGGAGCATCCCCCAAAGGAGCAAGGCCTG
TGCCTGTCACTAGAAGAGGOCTGTTGGCAACGGGGGCGTGACACCCCCAGCTACACTCTTCCTGTCCTCCCTCCAGTCTCTTGCCAC
GCCTCTCAGTGGCAGAACCCAACTGTTACCCTCAGGGTGAGGATGTACATTGATGCAGTTCATAAGGTCAGCCCCTTGGGCTACAGA
GCACAGACAGAGGAATGGATGGTGGCTGCAGAGGGCAAAGGGAACCTCTGGGTTTGAATCCTGGCTCCATCACTAGTTAGTTGTGTG
ACCCTGGTTAGGTTACCTGAACTTCTCTATTCCCTGGCTTCCTCATCTGTTAATTAAATGGGAATGACAGTAGCACCTACAACATGA
AGTTATCATGAAGATGAATGAGTAACTCGGGTAAACAATACAGTTGCTGCTATATAGAGGCCGCTAACAACTGTATTATGATGAAAA
CATCTGTTCTGTTATTCACAGTTTACAGATAAATTGAGTGAGGCACCCAAGCTCTCACATACATGCTAAGTGGCAGACCCAGAGTCC
AAACAGTGAATTCCAAGCCCAGAGTCTTAACCATTATACAGTGCAGTGCACCATGGTTAGAACAACAGCAACCCAACAAAGAACCTG
TCATTTTATCTCTTGTTCTCTCTCCCCCAGGCATCCTGTCCCCACCTGCACTCCACACACATCTGGAATCTAGCTGAAGTGGCTGTG
TGCCTAGGTCTTGGAATTGTGAAACCCATCCCTGAAAGCCTGGGTGATTTCTCCAGGGTTCCAGGAGCTACCTCTGTCCCCACCTTG
CTTCCTGCAGGCTGGGTAGCCAGACTCTTGTGAACAGGCCAATGGTGGTAGTTCCTCCCTTATTTAGATAGAAGAGAGCAGGCCCTT
GGACCTCACCATCATAGTAGTCAGAGCAGATGTAGGTTGGATGTGTTCAGAGAAGGAGAGACAGCCACTGGGATGCTCACAGAAGGC
TTTCTGGAAGGACAGTTTTGCTCTGGACTTTGAAGATTACATAAGTTTCACTAGTCAGAAAAAGACTGCTTCACAAAAGGAAGCCAT
CCTGCTCAGGAACAAACCTTACCTAACTTTACTTAGGAGGACAGCAGGTGGACGGCAATGAAAAACCTAGAAAAATCTCTCTTAGAT
CTGTCTTTAGCCACCCTGCCCCACACTTGGTGACCACTTGGGTACAGGTGGGAAACAGAGAGCCCACTCTCTGCACAAATTGCATGT
CAATTGCAAAATTGTGCCTATGGCTGTCACTTCACTTGCCAAGTATTTTCTTGCAGATTTCTGATATTAATGCATGGTTTTCAGTGA
AAATTTCTATGACATATGACTTTGGTTTTCAGATTTCAAGACCCTTTGTTTTGTGCAAATCAACGTTATTGATATCAATGATCAGAT
CCCCATCTTTGAAAAATCAGATGTGAGTAGTTGTCATTATTTGTTTTTTCTTTACTTTATTCATTTTACTTCACCCTTTCTTCTGGG
ATAGAGACACAGACAGTAGCTAGGAAAAGTGCCAGACTTAACAGGTAACCTTGGTCAGTGCACAAATAACCCAGATGCCATCTTCAG
TGGAACTTTTTGGGGAACAATAACATTTACTATTACAGGTGTCTGGGAACATTCATTTATAAACTCCTGCCAATAGCTCATTTATTC
CAAGGGCTGCTTAAAAACAACAACCACAATCTTTGTCAAATTGAAAAAAGGTGAATGGCGGCTTACAGTTTGTCATTATATATTTTT
TTGTGTTAGGACAATAAATAGATTTACTGCTAGGTCTCAAGCATTTTTAGCCAAAAATTTAATATTTGGAATATTGGCGATTTAATT
TGTAATAATTGGGTAAGAGAAAGGGAGAAGAAAAAAATTAATCTCTGACTTAAGCCAAAGAAAAATGTGCCCAAGACATTTGAGAGA
ATTAGAACTGGACAGAAAGAAAAAAGTCAATTTAGAACCTCAAGAAAAATGCAAATTACTGTTTTCTTGTCATCGATAGGGCGTTCA
GACTTGGTGATGACTTTATCATTGTTTCCCTAAGGAGCGAAGTCTCTATTTTGAAGGAGCAAGAAGCCTATTTGACTAGACTGTTAT
GTTAATTTTTGAAGTCAGTTATGAGGCAGCCCAGAGCCCTGTGATGATGTTCATAAATCAAATAGGGACAAGCCTGCAACAAAGAAG
GGAATTGATTTATGAGGGGGCTAGGGGTGGGAAGAGGTGTGAGTGGATATGTCTTGTATAACAGGCACTGAGGTTCAGCAGGCATCA
AATAACCCATTTCTTCTCAGCCTCCATTCTCTTGGGCTTTTGATCCTTTCCTAACTTTGTGACCTCCTCGCCTTTTATCGAGACAGT
GTTTTTTAGATTGAGGGTAAGAATCCTGCACTTGAGGTCTCAGAGCTTAAATACTAATCTGGCTACAGAGAGTGAACCTGCTTGTTT
CAATGCTGTGGTCCTCTCTTCTGAGACCTACTTATAACCTTCTTTAGGATCATGATGCCAAGAGAGATAATCTGGGGGATTTTCAGT
GGAGTGGGAGATGGTCTAGGCCAGTGTTCAAGAGCATAGGCTTTGGAGTTGCACAAACATGGCTTTGAATCCCAATCCCTTTGACCC
ACTAGCTGTGTGACCCTTGGTGGGTTTTACTTCTCCAAGCCTTTGCTCCTCGTAAGTGGAGATGGTAACATAACTACTCCGTAAGGT
GTTTTGTTGATTTTCTTTATTGAGCACTTACTATGTGCCAAGAATTTAGGAGCTTTACATGCATTGTCTCCCTTAACACCCACAGCA
AACATGAAGTAGGTTCTATTACTATTGTCTCCATTGGACAGATGATGAAACTGACACTTAAAGAAGTGAAGTAACTTGCTTCAAGCC
ACTCAACTAATAAGAAGCAAGCCTACAATTCATATTCAGGCTGTTTAACTCCAAAGTTCAAGCTTTTAATCAACCTCTATTCATTGG
TTGATTTAAGAATTAATTAAATAATGTATATAAAGTGCCTACCACCTGATAGATACTCAATACATGGTGGCTATTTATATCATTACT
ACTACAATAATTAAGTTATTAAACAAATTGGATATGGGAGGGGGAATTTGTTTTAGAATCCTACCAGTAGCTCTCTGAGGTATTGCG
TTCTTTGGTACAATGGATATCAGCTCATGAGTATGACTGATGTTAAAATCAACTGCAATATATTCATCAATAGGAAAAAACATGCCC
CATGAAGATTGATGCAAACTATGACTTGTGTCTATTCTGAAACCAGAGAGATAAGTTTGTCCTTTGGCTTTGATGACAGCAGACCTT
CTCATTGTTTCCTTCTCCATTTTCTCCTTAGTATGGAAACCTGACTCTTGCTGAAGACACAAACATTGGGTCCACCATCTTAACCAT
CCAGGCCACTGATGCTGATGAGCCATTTACTGGGAGTTCTAAAATTCTGTATCATATCATAAAGGGAGACAGTGAGGGACGCCTGGG
GGTTGACACAGATCCCCATACCAACACCGGATATGTCATAATTAAAAAGGTAAATAGCCCATTTATTTAATAGAATTTGTCTTTGTT
TGTGGGTTAATTTACTAATGACAGTGTGAAGACCTATAAGCAGAGGTGATGTATTAGCAGCCATCTCTCAGGCATGGTGATGGGTTC
CTGGATTCCTGGATTCCTCTTAGTCGTTCCTGTCTTCCTGACAGACGGGAAGTCAGGATTCTAGACCTGGCTCTGTTGCTAGCTAGC
CAAGTAACTTTTGCAGGGAGGTAACGTCATATAAAATATATAAATTGCAGGATGAACAATTTTTTAGTATAAGTGTGTTCTATGCAA
TATTTGGGATGCACTTATACTAAAAACTTGTTTGTCATTTATCTGAACTTCAAATCGAACTTGGTGTTCTATGTTTTTATTTGCTAT
TTGCTAAATCTCGCAACACTCTTACGGTCTGGGAGTGGCACTCAGGGTCTCGCTTTCACTTCCCCCTTTGCAAATGTGAGGAGTTGG
GTTGGATGACCCCATGGGCCTTTTAGGCTGTAAACCCTTTATTCTAATGTGCCAGGCAGAGATACCTTTTGAATAATGCAACTGCTG
GGTCTCCTTCCAAGGTTTAATAGACAAGTGTCTTTTTAATCCCCCTCTCCGCTGCTTTACTTTCACCTTTTACTGTATTGCTAAGCC
CACGGGCTCTCATTCTGTCTCTCCCTCTGTAAGGACTGGATAATCTGGAGTCAAAGTCTTAATTACCTTGGTTCCTGTCTCTTTGTA
CAGGATTCTCCCAACCCTCCCTCAATCTACTCCTGTCCCCTGCCATGTATACCTGAATCTTGACTCTTTTCTCTGTGGCTGCCTCAT
CTGTGTTTAGAGCATGTCATGGGTTTACCAGATGACTCAATTGGAAGCAGCACTCAATGCCGTCCAATGTACTTACAGTTAAGATAC
TTTCCTAGGAAAGAGAGTGAAACCTCGCTGTTAAAAAGGGCCAGAGAGAATCTGCAAGGGTGGCTAATTGTTTAAGGTTTGCAGTGC
AGTGTGCTGGAAGCTGCAAAGGAAAGCTCTGGACTACAGTTTTGAGCCAGAATAAAAGAAAACTGAAAGAGGCTAAAAATGAAGACA
AAATAGAATAATTTCACAGAAAATGGACAGAAGCACAGAGAAAAAAGCACTAGTGCTGAAGCAAGCCCTAGGACAGAGCCTTGGAAT
TGTGATCCTACTGTTACCAAACATTCCAGCTTGAACCAAGGGCCCTGCTGCATTTCAGCAGAGAGGTTGCTGTGGACCAGCTCAAAG
CCTTAGCCTTGTGTGTGAGTCTGACATTTGGAAAAGGTACCATGACAACCTCCCCTTGTGCTCACAGCAGTCTGCATGGAGAGAGAA
TACCACAGAAAGAAAGGATAAAGGAAAGGGCAGTTTTCAGAGCTTGCAGTGGGTTGGTGTGTTCCTTTGCAGGTGACGGGGGCAGCT
TCCTTTTATCCTTTTGCAGGACCAGAGCTCTCTAGGAATCTAAGGGAAGATAGAGGCCCATCCATCCCAGTATGAACAAGGCCCGTA
TGTCATCTTTTTTTGAACAGCAGTTTAACTTGGCCATTTGTGGTTAATGGGCCCTTTTAGGGAGATGTTGGCACCTATCTCAATGAT
TAGCTGTGGGTGAGAGCTGTAAGTATCCTCCCTCACAACTTGACACGAGAGGTATGCTACAGTAGAGTAGGCCTCTGACCAAACTCT
TTGTCACATCCTTACTTTATATGTGGTTCTATTTCAAGTGTCTATTATGTCTCCTGGCATATATTAGAGGTTCAATAAATATTGCTT
GACTTAATGAATAAATGAATTCTGTCCCATATACAATTATGAACAAGCAGACACATAAGTGATCATCGAACCTGTGCTTTAGGCCTG
TGACATCCCAGCTCTCTGTCCCTTGGGCACTGTGATAACTGTCCTTGAGCAAGTGGATTTGTTCAACCATCCAGAGGCAGGTGATAA
CTGGCAGAACAGAACTGAGCCCTGGAACTCTGCAGGGTACAATCTGCTGGCCAGGCCAGATATCTATACTCCTGTTCACAATGACTA
AGGGATACACTATACTCGTAACCAAACAGCACATCTTTTTTTGAGACAGATGCAGTGCCGCAGTCATAGCTCATTGCAGCCTCAACC
TCTTGGGCTAGAGCAATCCTCCCACCTCAGCCTCCTAAGTAGCTGGGACCACAGGTGTGTGCCACCACACCCAGCTAATTTTTTATT
TTTGTAGAGATGGGGGTCTCCTTATGTTGCCCAGGCTGGTCTTGAACTCCTGGGCTCAAGTGATCCACCTGCCTCAGCCTCCCAAAT
TGCTGGGATCACAGGTGTTTGAGCCACTGTGCATGGCCCAAAGTGCATATCTTGGACACCACTCATATCACCAAGAAAATTTATTTG
TAAGATCACTTTGCTAATTAACAAACTTTCTAGTTCTAATTGAGATAGTGTTGGCTGAAGGACTATCATAGCTACCTCTGGGATATA
GATCTCGGATTAGGTTCTTTCAGATGCTAAGCAACAACCAACCCTGGGCTGTGGATTCTTGATTAGGACACACCAAATGTTAGCCAA
TTCCCCACTAGGATATAGAATAAAAATATAACAAATGTATGTAGCCATACTATTTGTTATTGAAGTGTTTTATAGACTATATAATTT
GGTTTTTTATGTATGGCTTTGTCTTCTAAGTCCTTGCCTATTAAGCCATCAAGGGACTACCACTGTTCTGAACCACTGTGTTAATAA
TTTTTAAAAGTTTAAGTTTGTTAATCACAATTATATCATTAACAGTTCATCTGAATTACATGTGCTAATCACAGGTAAAATTTTCAC
TGAGGGACTCATACGTGGCAACTATAGTTTGTTTGCATAAACTCTCTTGCTTCTCCCACTGATTCTGAGGGAGGCAGAAATTCAGGC
CCACAGCAAGGTACAGTCAGGCCTTCTGGTGATGGCAGAGCCGGGAACTTGACTCCAGGCCTCCACCTCCTCTCAGGAAATAATTTC
TGTGAGTCCTGGTGTTGCCACTTACTAGCTTGTGGCCTTGACCAAATGATGTAACCTCTCAGGCCCCAGTTTTCCAATCTGTAAAAA
TGGGTAAAATGGTAGCACCTTTCTAGTAAGGATGTTGAAGAATTAAATGCAAAAGTATGTGTATAGTTCCTAACACAGCATCCAGCA
GCTTGCAGACTTAAACACGTGGCATCACTTGATTCTAATTAATTAGAGCTAGGATTCTACCTGCGGACAGGTATGCTTTCCCCTGTT
GCCTAAGGGGGCCAAACTTGCCTAATTCAAGAGTCAGATGGTGCATACTGAGGACTGCGGCCTCAGCCTCTCAAGGGGAAAGCCTGG
AAATGAGTAATTTTGGCAAGTGCCCAAAGCAATCCTTCAGATTAAACAACTGTCAGAAATGCTCCCTGCTCTGTATTTACTTCTGCC
CAAGCTCCAGCCTCTCGTGTGCTTGGTTATCATGCTCTTGGGGCCAAACGTGTGTCATGAGCAGGAAAGCTATTTCTTTGAGCCCTG
CCTGGCTCAACAATAACTCTCCTGAGATCCTGTTTCTTGAAATTCAGGGTTCTGCCTTATTTTAACTTGCAAATTTTGTCACTTCTC
TACTATGGCTGATCCTTTTGAATTCTCAGTTCACTAGCTATTTTTAACCAGACCAAATGTCATCTGGAAAACTAATGTGCACACTTT
GATATCATCAGTGATCGCTAGTGAAAATTGTAAATATCCCAGTCTCAACCCCTCTCCTTCAGCATTTTCTTTGCCTCCAGGTTGAAT
CTAAATCTCTAGGTTTGACCTTTCAGGCGGTTTTGTGCACAAAAGGAATCGTGGTATGAGCAGGTTTTTTCAGTTAACTTGAATATG
TCTTGCCAGATGGGACTGAGGCCATTCCAAGAGGCAGTGACGGGTCTCTTGACTTGGCCTCACAGTCATATCCCCAGGTCATGAAAC
ACAATGCACGTGGCCGGACAAGGCTTGTTCTTGGCTCAGCTCTGCTTATTACCATGTGCTAATTTAGCATCTTCCGTTTATAATGAT
TTCTCCAGAAGATTTGGTTTGATGTTTTTCCAGGTGTGGAAGTTATGCGGATAGAATCCTCAATGGCAGGCTGCTGCTTTTCATTTG
TTCTTCCATCCAGTGTTTAGGGAACACTGACACTGCCTTCTAGCTAGTCTTCCCGCCCCAGTGTGGATCACCTCGAATCCATCCCCC
ATGTAGCTGCTCCTGGAGTTATCTTTCTAAGACACACATCTGACCTTGTTAGTCAAGGATGAAGTTCCAACTCCTCAGCTCGCCGTA
CAGTCTTGTCTTTCCACTGTCAGCCTTGCACGTTCTCTTCTGGTAATACAGGCTGCTTCTCATTTGCACACACAGTGTCTCATTTCT
TTAAACCATTCCCTCTGGGGCCACAGTGATGGGATGAGGAAAAGCCAGGGAGGGTGCTTAGGACCTGCTTGGTGGTGAGGATGGATG
CATGTGCTCTGTCTGAGCATATGGACAACTGTGCTGCCTGCCTGGGCCCGGACTTTGCTGGACTGGATAGGAACAGAGAGCTGTGTC
CTTTGTCCCCATCACAGGCTCATTCTGGGGGCTGCTGCCTTCCTGCTTCGTCTACTAGGCTTTTATCTCAACTGCACAGTTCTGTCA
TCCCCTGAGACTGGGAGTGGGTCCTGTGAGTGAGAAACTCCAGCTTGTTCTCCCCACCTCTACTTCAGGGGGAGAAGCCCTAATTGA
ACCGTGGACCACGTGACGTGTATTGGACCACGTCACGAGGTCGACGAGGGCCAGGCCCTGCAGACAGTGGGTGCTTATCACTATGAA
CAACCTCTTTTTTCACACTTAGCTGGCTTACATGATTTTATCTCTTATATATTTGAACAAATTTCAATTGATTCTTCTTTCTCAACC
CCGAGTCTTCACACACAGTTCCCACCCTTCTACGTGAAAAATTGTCCCACAGCACTTATGAACTAGAGAGTGAGTCATGATGGTGTG
TGTTGGTGCATGCATGCGGGAAAGGGGATTCTGGAAGGTGGGGATCTTCTCCAGAATCTCCTCTCCATGGAGGCCCTCAAGGACCAG
GCCTTCCTGGTCTTTGTAGTTCCTGACCAGCCCACTGTGGGCACTATGTGTCAGGGAACAGATGAGAAGCAAAGATGAATATGATGC
AGTTCTTCTTCTCATGGAGCTTAGGGTCAGTGAAGATCTAAAATACATCAACACTTAGATCTATTAGGTGCTATGAGACTGCACAGG
ATGTGCAGCTTGTCAGTTTGAGGATGGAATGTAGGGATCAGGAAGCCGACACGGAGAAGACAGCAGTAAGCCCACTCCTGAAGAGGG
GGAGTACTCTTGGGTAGCCCTGTTAAATTCTGCCTCATATTAAACCAAAATTGGTTTCTTGATGTTCATTCCTGGTTCCATCTGGCA
TGTCCTCAGCAGATCATCTGTCATCTCTGTTTCCCTTTCCCATCCTCCATGAGTTTAAAATCTTTGATTTGGGGGCTTGAGTTTTGA
GGCCCTTGGAAAATCATTTCCCATAAAAAATGGAAATCTCTATGTAAGCATTTCTCAAGCTGTCACCTGAGAATGTCTTGGGAGAAT
CAATACATTTCTGTTGAATAATGAAGTGTCCCACAGTTAATTTAAATTTGAGGACTTCCGTGTTCCAGTTCCTCTTCTAAGACATAT
TAGCGTAGTGCAAGCTCTGGTAAGACCTGCAGAAAAGACTCTTTCATGGCTCTGTAATCATTCTGAGAAGGTTTTTGCTGACACTCG
ATTGAGAACCACTGCTATGAAAGTTTGTCTTGTTTATAATTGACAAAAATTGTATAGGAGGACAACATGTTTTGAAATATACATACA
TTGTAGAATGACTAATTAGAGCTAACTAGCATCTGAATTACCTCACTTATCATTTTTTGTGATGAAAACACTTAAGATCTACTCTTC
TGTGATTTTAAAGTATACACTATATTGTTGTTAACTATAGTACCACGTTGTAAAATAGATATTTTAAACTCATTCCTTCTAACTGAA
ATTTTGCATGCTTTTACCAGCACCTCCCCAATCTCCTCATGCCCCAGCTCCTGGTAACCAACATTCTACTCTCCAAGTTCAACACCT
TTAGATTCCACATACAAGTGAGAACATGTGGTATTTGTCTTTTCATGCCTGGCTTATTTCACTTACCGTAATATCTTCTAGGTTCAT
CCGTGTTATCACAAATGATGGAACTTCCTTCCTTTTTAAAGCTGAACAGTATTTCATTGTGTGTGTGTATATATATATGTGTGTGCG
TGTGTATGTATGTGTATGTATGCATATATATACTGCATTTTGTTTATCCAGTTGTTTGTTGTTCGACACTTAGGCTGATTCCATGTC
TTGGCTATTGTGAATGATGCTGCAGGGAACATAGGAGTGCAGATATCTCTTTGACATACTGACTTTATATACCCAGTAGTGGGATTT
CTGGATCGTATGGTAGTTCTATTTTTAATTGTTTGAGTTTCCTCCATACTGTTTTCCACAATGGCTATTGTAATTTACATTCTTACC
AACAGTGTACAGGGTTCCCTTTTCTCCACATCCTCACCAATGTTTGTTTTTTGTCTTTTTGATAAGTCATTCTAACAGGTGTGAGGT
AATATCTCATTGGGGTTTTAATTTGCATTTCTCTGATGATTAGTAATGAGCATTTTTTTCCATATACTTGTTCGCTATTTGTATGTC
TTCTTTTGAGAAATGACTATTCAGGTCCTTTGCCTAGGTTTTAAACAGGTTGTTTTCTTGCTACTGAGTTGTTTGAGTTCCTTATAA
ATTTTTTATATTAATCCTTTTTCTAATGTGTATTTGCAAATATTTTCTCCTGATCTGTAAGTTGCTGCTTCACTGTTTCCTTTGCTA
TGTAGAAGCTTCTTAGTTTGATGCAATCCCATTTGTCTATTTTTGCTTTTGTAGTCTGTCTTTTTGGGGTCACACCTAAAAGATCAT
TGCTAGGCTGGGCATAGTGGCTCACGCCTATAATCCCAGCATTTTGGGAGGCTGAGGTGGGTGGGTCACTTGAGGTCAGGAATTTGA
GACCAGCCTGGCCAACATGGCAAAACCCCCTTCTCTACTAAAAATAAAAGGGTGAGTTTCCTCTATCTCGTGAGATGGTTTGCCCTT
CTGTCATAGGATAACACAGCACAAAGTCCTCAACAGATGCTGGTGCCATGTTCTTAGGCTTTCCAGCCTCTAGAACTGAGAGTCAGA
TAAACTTCTATTCCAGCCAGGCATGGTGGCTCATACCTGTAATCCCAGCCCTTTGGGAGGCCAAGGTGGGCAGATCACTTGAGGTAA
GGAATTTGTGACCAGCCTGGCCAACATGGTGAAACCCTGTCTCTATTAAAAATACAAAAATTAGCTGGGTGTGGTGGTGGGTGCCTA
TAATCCCAGCCACTTGGGAGACTGAGGCAGGAGAATCACTTGAACAAACCTGGGAGGCGGAGCTTGCAGTGAGCCAAGATTGCGCCA
CTGCACTCAAGGCTCAAGGCTGGGTGACAGAGCATGAGGCTCCATCTCAAAAATAAAAAATAATAAAAAAAGATCATTGCCAAGACC
AATGTCAGAGTTTTCTTCTATGTTTTATTCTAGTGGTGTTATAGTTTCAGGTCTTATATTTTGTCTGTAATCTATTTTTAACGGATT
TTCATATATGGTATGAAATAGTCTAATTTCATTCTCCCACATGTGGATATCCAGTTTTTCCAATATTATATATCGAATATATGAGAG
TTTAAGAAGGATAGGGAATGAATCTTTTAAGGGAGCTAATCACCCCAAAATTAATTTTCTCTCTTTCTCCTACACAGCCTCTTGATT
TTGAAACAGCAGCTGTTTCCAACATTGTGTTCAAAGCAGAAAATCCTGAGCCTCTAGTGTTTGGTGTGAAGTACAATGCAAGTTCTT
TTGCCAAGTTCACGCTTATTGTGACAGATGTGAATGAAGCACCTCAATTTTCCCAACACGTATTCCAAGCGAAAGTCAGTGAGGATG
TAGCTATAGGCCTAAAAGTGGGCAATGTGACTGCCAAGGATCCAGAAGGTCTGGACATAAGGTAAAGGGCAACAGTGCTGGGCAGGC
CTGGCTGCGTGGTCACCAAAGCCAATGGGGAGGAGGAGGAGCTGGCCTGCACTGACTCCAGGGCTGACCCTCACAGCATACCCAGCA
ATGATGAAATTGGCTTTGGTTTAGGAAGCTGTCAACACCCTGGGGGCATTCCAAGGGATGGTGGTTCATCAATAACCATCTAGGCTC
AGACAAGTGGGAAGCAGCATGAGGTTCCATTCCAGCCCATGAGCTTCAGGTCCCAGCTGAGAGCTGGATGGTTCAGGAAGTGAGGTT
CACCTCTGAGACACTAGAATAACCAGGCCCTGTGTCTAAGCCTGTTATCTAGGCTAGGGTTTAGGAAAGGGTTCCAGCCCTAAAGGT
GTATTGGAGTTCTAGACAGAGACCAAAGCCCAATTGGACAGACCATGCCAACTTACAGGGCTGAGGGAATCTTCTGGATGTAGGATA
CATAGGTTCCTTGGCTCCAAAGAGCTGATCTAGGGCACCCCTGGAGAAGCTGGTGCTCTGGCTGAGGTGGCTAAACAAAATGTGTAA
ATATTTCCATCTTAGCAATTCCTTTTTTTCAGTTCAGTAAGACCCACACAGATTTGCCTTAAAAAGTACAATGATGATTAACTTAAG
AAATATATGTTTGTATATATCTGTATAATATCTATAAAATCTTTAAGATCATGTAGTTAAGACTTGAACTCAGATCTTTCGCTGCTG
TTGTACATTACACCTCCTGTCTATGGCTAGGAATATTTCAGCATGACTTAATCACGCGTGTGGCTATGAATCGCAGAAAACAATTCC
TTAAGTTTCTCTAAACATCTGGAGGGAGTCTTGCCTCCCATTGAGCTGTCCTTCAGGCACTTGGATTCACTTCTAAGACCTTTATCT
GATCATTGAGAAGCAAAGTTACCACCTGCAGTTTTACTCTAACTACTTCTGTAGTTTTACAGAGAAGAAAGAACAGTAGGGGATATA
CTGTATGGTTTAGTGTAGATACTACTGGGTCAAAAATTATGAGAAGACACAAAATGTTTTTAATCTTTATACCATTTATTTAACAAA
AGTTCCTATCTTCTTCATGAAAATGCTATAAAAACCATTGGTCAAAGACGTGAATAAAAAGTTTATCAGTGGGAAAAATAAAGGCAT
GTAAGAAAATTACTGTGATGAAACAAGGAAAGGATACATATACCATTAAGTTGAGGTCAGGAAGAGGAGGTTAGCAAGGGAGACTGA
GAAGAGGTGTTCTGTAAGGTACAATTCCAGAAGACAAATGAAGAAAGTGTTTCAGGAGTAAGGAATAATCAATAGTTCAGACATGAG
TAATAAGTCTGTTAAGAATTAAGGATGACTCCAGTGTTGGTCTGAGTAATTTAATTCTCCAGCTCCACAAACTTAAAATCTGTTAGC
ACCCAATGGATTTGAATCCAATCCTTCAGCAAATATTATAGATTCTTTTATTGAAATATATTCAGAATCCACCTGCTTCTCCCTGCT
TCCAGCCTGCTCCAAGGCACCACCATGTCTGTTCTGGATTCGTCCTTGCCCCTGCCGTCCCCCACCCCACTCTGAATCTACTCACTG
AATTGCAGTCAGTGACCTTTTAAAAAAGTCAATCAGATCATCTGACTCCTCTTTCAAAACCCTCCAAAGCATTCCATCTCTTTCAGG
ATGAAATCCCAAGTATAAAGGGTTTCATACTCATTACCCATGTATATAACCTAACACAGTGTTTGGCATATGGAATATACTAAAAAA
ATACATTGTGGGAAATTAACACATCATACTTTTTTAATGATTCTCTTGTCCTTTGATATGTAGGTTGCTTCCAGTATTTTTTATTAT
AGAAAATGTTTTAAATAGATGTATGCATGTAATTCTTTTTAAACTTTGATTAAATTGGGTTTTTTTAGGTAGCATTTCCAGGAGTGA
AATTATTTGGTCTAAAGATATGGACATCCTCATGACTATGATGTGTTGCTTTTTAAAGGACTACCATTGGTTTTCCTGTGGTTCTAG
TCTTGTCTGTCTCAACTTCATCTTCCAAACACCTACCAATGAGCTCCGTTGAAAGCACAAATATGGCCAGGTCACCTTCTTGCTTAA
ATCATTGGCTGGCTGCCCATTGCTTCTTGTGATCTGGCTCTTCAAATCTCTGTGGCTTCACCTTTTGCCACCTTCTGCTTCCCAAGT
ATACCCCAGCACTGCTGAAATGCCTGACCCCACACTCACCATGCTGTTCCTTGGCCCTTGGTGCTTTATCTCACACTCTTCCTTCTA
CTTGAAGCGTCTTCTTCTTGCCTTAAACATCTACTACTTCAGAAAGACTTTGCTGATTCCTGTAAACTTCTCTTGGATTCCATAGGG
CCCCCTCTGTTGTGTTTACCACATTTTATTGGAATCAACTGTTTATCTGTCTATCTCACTTGCCTAAGAACACCTTTTTGTGATCTC
TTCCCTTGGCCCACTGCCTGATCTTCCTCAGCACTCAATAGATGTTTGTTGGATGAGGAATTCACTGTGTTACCAGGGTCTATGAGT
ATCCACCCATCTCGCCCAACGTTTCCACCACTGGGTGTTCAAATTTTCTAGGTGGGTGGTTTGCTAAGAGGTTTTAAAAGATACTTC
CAGGTTACTTTAATTTGAGTTTTTGATTGCCAGAAAAGTTTAACTACTTTCCCCAGAAGTTGTGAACTATACCCTTCCTAATACAAG
CACAAACACCACATTGTAAGGATGAACAAATCCTTTTGAAGACAGATGAAATGCAAATAAAGCATTAATAATATGAGATAATAGATC
CTTTAATATGTTTTAAATAATATACAAGTAATGCTGATAATGATAATCTCAGCATTATCATTTGTATATTATTTACAACAATATTTC
AAGGATGCACAAACATATTTAGTTGACGCACAAACTAGCTTCACATTTTCATGCAAAGTAATGCAGGCTTTCTTTTGCTCTTTTCAT
TTGATGAAGCTATTCACTGAGGGGAGACACAAGAGGTTGGCTTAAAATTGACCACGTGACTGGTGAGATCTTTAGTGTGGCTCCATT
ACACAGATTATCAGAAGATACATGGGTTGAAATGGGAGGGAGGATAAGAAAACTGACAGGGCCAAAACACCAGAATATCACAACCTT
AAAGTGGAAGGATCAGAAGGGTACAAAATATAAGGGAGTGCTCATATCTTCCTTTTTTTTTTTTTTTTTTTTTTTTTTTTGAGATGG
AGTCTTGCTCTGTTGCCCAGGCTGGAGTGCAGTGGTGCGATCTCCACTCACTGCAACCTCCACCTCCTGGGTTCAAGCAATTCTCCT
ACCTCAGCCTTCCGAGTAGCTGGGATTACAGGCACCCACCACCATGCCTGGCTAATTTTTGTATTTTTAGTAGAGACAGGGTTTCAC
CATGTTGGCCAGCCTGGTCTCAAACTTCCGACCTCAGGTGATCTGCCTGCCTCCACCTCCCAAAGTGCTGGGATTACAGGTGTGAGC
CACCACGCCCAGCCCCTTTGTGGTTTTGTTAAAGGTGAAAAACACCTGTGCCCCCAGCAGGGCTGCTGCTCACTCTGTGGTTTCCTG
TAGGGGCTATGGCCTCTTCTAGAGAGTTCTGTGTCCCTGGCTGAACTACCTCCCTTCTTCCAATGTGGTTGTGGGTTTCCCCCAGGA
GCTCTGAGTGGAATCAAACCACTGACCTGTTCACGTAGACCACCACTTGCTCCTTCAGCCTTAAATTGTAACTCTCATCCTTTTTGA
AGCAGAAAATGTTTTATGAAATGTTCTCTACTTTGATGTGTGTGAGAATCCCCGGGGAGCTTGTTGAATAACACTAGGAGTCCACCC
CTGGAGTTTCTGATTCCATAGGTTTGGGTGAACAAGGGGTATCTGTGTTTTGACGAAATCAGGTGAACTTTTGAAAGGAAAGACAGA
AAAGCCCAGGATGTTTGAGCCATTCCTGTGTAATCTTTGTATCTTATTTATTTTAGTTGTGCTGATATATATTGCCTGCTGGCTCCA
ACTGTGGAGTTCCAGAGAACTTTTTTAAAATAAAAATAAACACATAGGAATAGTATGTTTACCCAATAATAAGCCTGGCAACTAAAA
AAGAGTTTCTTTGCATTTTCATGGAATTTTCAGACATACAAGTAGGAAATCTGTTGATCCAATGTTGACCAGTCTCTAATTAGCTAG
TAAATGTATCTGGCAGGTTTACCAAAAAAGAGAATTAAACAAATTACTCAATAAATATGTTTTTAGACTCCCCCACCACAGCCCCGG
AAAAAAACTTTCAAAATGTGAGGGCCAGGGTTGAGAGCTGAATTGGAGAAGAGAGACAGGAATCATAAAAACGGCCCCTGCATGTGG
AAAGATTAAGAAAAACAAAATTACTTCAGTCAAGTCAAACCAAAAAGGGTAGAATGCAGAACGGTGTGCCAGCTTGGGAGGGGTGTG
TGTGAATGGACAGGCCCTCTGCTTCTGTCTCTTGCTTGAGTGCAGCACATTCCACTATCACATTGTATACATTTAACTCTTGATTTA
AAACCTCCCAGTGAAGCAGCAGGCAGGTTCTGGCTGAGCCTTTATGCTGTGCACACCCCTTTCTCATACCATGGGGATCAGAACTTC
ACTCTGGATGTGTCCCAAGAGTTTCTCACATTCTAGAAGTTTCAGAAATCAAAACCAGGGTCCAAAACCCTGTTCTGTTTTTAGTTT
CTGGCACTGACATGATATAAATAGATACAATAACTTCGAGAACACAGTGGCCTTTTCTTCTTTGGTTAAGTATAGCCATGGACATAG
ACGATGTTGAGAAGAAAATCAAGAAAACAGGGTTTACTGGAAATGAGCAAGAAAACATTACTGGAATCAAACAAGGTCAGAGTCAGA
AGTTGTAGAAACAATGTCTGTGATTGGTGTTGAGGAGGACATGATAACAGTCAGTGTTTATGTTGCACTGACAGCCCCATACTGAGC
ACTTAGTGTAATAACTTACTCCTCATACAACACTATAACGTAGGTGCTATTATTGACCACATACTGTGAACAAGGAAACCAAAGCAC
TGAGAAATTAAGTAACTTGCCCAAGATTACATAGCTACCAACCAGTTATGCTGCAGATCCAAGTTTTTTACTCCTATCATATAGTAA
ATAATGTCTGTGGAGCCTTTTACTATTTTCCAGAACATTCACTCATATGTGGGATTCTTGCAAAGCCACACAGCTGAAGAAAATAAG
CTTAGAAAACTCTACTAACATGCCTAAGGCCTGCTAAAGAGCAAGTGAAGGTGTGAGTGCTAGAGTTAGACCTCTTGGATTGAAAAC
CTCTCTGCCTTGGGCAAGTTACACAACTTCTGAGCTCAGTTTTCACAATTATTTTGAAGTAATATATGTAAAATATTAGCACATATC
TCTTAGTTATTATTATGATGGTCAGTAAGACTTTTTCACCAGTAATGAAACTAGGTCTTTGATTCTAAAAGCTTGTCGTCTTTCTAG
CATCTCAGTGTACCTAACTCCTTGAATTTTAAAGAAAGGGAAATCTTTCTTATTAAGAGGAAAATGAGTGAACTGATCCCATGTTTC
ATCATTCAATTCCTTAGGGGGGTCTTCCTTGAGCTCTGTGTCAGAGTTCCACCTGATCCTTATGGATGTGAATGACAACCCTCCCAA
GGCTAGCCAAGGACTACACGGGCTTGTTCTTCTGCCATCCCCTCAGTGCACCTGGAAGTCTCATTTTCGAGGCTACTGATGATGATC
AGCACTTATTTCGGGGTCCCCATTTTACATTTTCCCTCGGCAGTGGAAGCTTACAAAACGACTGGGAAGTTTCCAAAATCAATGGTG
AGTTGTCAGAAATACATGGAAAAAAACATAGATGGATGATGAAAGTAGGTTTTATTTAACTTGGTGGGTTTGCTGTACTCAGCAAAG
AGCTTTCTTTCATGCTTCAGGAAGGAAGTTCATGCTTTGGAAGCTTGTACAGGGAAGGACTCCAGAGGGGAAAAAGCCCCAGGATTT
CAAGGGAGGAAAGATTATGACCACACAGCATTCTCTTGGTGTAGATGAACAGTTGGATGACCTAAGCATCATTTGCAAAATTTTCAA
TGACAGATGGAAAGCAGATTTTATACAAATTTTTAAAAACTTTGAGATAGATATATATATTCAAAGTATACAATTTTATGAGAATTG
ATTACATGTAAATACCACTGAAGCCATCAGCACAATCAAGACAATGAACATTTCCACCACCTCCAGAAGATATTTCATGCCCTATTA
TAATCTCTGCTTCCCACCCATCCCCTCTCCCCAGGCAACCATTGAAGTGCTTTCCATTACTATAGTTTTCATTTTCAATAATTTCAT
CTAAATAGAATAGTATAGTATGTATATTTTTTTTGGTCTGGCTTCTTTCATCTCAGTATTTTGAGATGTATCGGTGTTGTTACATGC
ATCAGTACTTCATTCCTGATTATTGAGGGGTAGTATTCCATTGAATGACTATACCTGTTCACCTGTTGATTGTAACTCCAACTTTTG
CTATTACAAAGCTGCTGTGAGGATTTGTGAACAAATATTTTGTAGGGACAAAGATTTGCAGTTTTCTTGGTTAAATACTGAGGAGTG
GAATGGCTGGGTCATATGGGAGTTATATGATTCAGTTGTTAAGGAAAGGTTGAACTACTTTCCAAATTGTTCTTAACATTTTACATT
CCCACCAGTGTGTGAGAGGTCCAGTTGCTCAGCATTCTCCCAGCACTTGCTGTGCTCAGCCTCTTTAATTTTAGCCATTCCAGTGGG
CGTGGAGTAGTATCTCATAATGGTTTTATTTGTATTTCTCTAATGACTAGCGGTGTTGAGCATCTTTTCATATGCAATTTGCCATCC
ATCTTCACTGGTGAAGTGTCTTGTTAGATATTCATTTTTTAAAAATTAGGCTGTTTGGATTCTATTGCTGAGTTGCAAGCATTTTTA
ATATACATAGATGATGTGCTTTGTTAGATATATATCTTTTGTAAATATTTTCTCCCAGGCCATGGCTTGTCTTTTCATTTTGCAAGT
CTTTTAAAGAGCAAATTTAAAAATTTTGGTATCTATTTTTTTCATGTTTTATTCATGATTTGTATGTTGTGATGCTTAAATTTTTGC
CAAACTTAGGGTCCCTAAAGTTTTCTGTTTTTTTCATTTCCCAAATTTTATAGTTTTAGCTTTTGCATTTAGCTCTATGCCTTTTTA
AGGTAATTTTTGTATAGGCATACCTCATCTTATTGCACTTTGCTTTATCGTGCCCAATGGATCTTCCATTTTTTTATAAAATGAAGG
TTTGTGGCAACAAAGTCTCTTGGTGCCATTTTTCCAACAGCATGTGCTCACTTTGTATGTCTGTCACATTTAGGTAATTCTTAAACT
TTTTCATTATTATATCTGTTATGCTGATCAGTGATCTTTGATGTTATTATTATAATTATTTTGGGACACCCGGAGCCATACTCACAT
AAGACGACAAACTTAATCTGTCAATGCGACGTCGACGTACGTTCTGACGCTCCACAGACTGGCTGTTTCCCCATGTCTCTCTCTCTC
CTTGGGCCTCCCTATTGCCTGAGACACAATATTGAAAATTAGGCCATTAATAACCTTAAAATGGCATGTAAGTGTTCAGGTGAAAGG
AAGAGTCACACATCTCTCACTTTAAATCAAAAGTTAGACGTGATTTAGCTTAGTGAGGATGTTATGTTGATAGCCAAGATTGGCCAA
AAACTAGGCCTCTTGCATGAAATGCTTAGCCAAGTTGTGAAGGCAAAAGTTATTGAGGGAAATTAAAAGTGAACCTACAGTGAACAC
AAATAATAAAAAAGCAAAATAGCCTTATTGCCATTAAGGGAGAAAGTTTGAGTGGTCTGGATAGAAGATTAAAACAGCAACAATAGT
CCTTTAAACCAAAATCTAACCTAGAACAAGGCCTTAACTCTTCAATTCTGTAAAGGCTGACAGAGGTGAGGAAACTGCAGAATAAGT
TTGAAGCTAGCAGAGGTTGATTTATGATGTTTAAGGAAAGAAGCTATTTCCATAACAAAGTGCAAAGTGATGCAGCAAATTGATGCA
GAAGCAAGTAATCCAGAAGATCTGAGATCCTTAATGAAAGTGGCTACACTAAACAAGCTTTTCAATGTAGATGAAACAGCCTTCTTT
TGGGAGACAATGCCATCTTAGACATCCATAGCTAGAGAAGTAAATGCCTGGCTTCAAAGCCTCAAAGGACAGGCTGGCTCTTGTTAG
GGCTAATGCAGCTGGTGACTTTAAGTTAAAGCCAACCCTAATTTAGCATTCCTAAAATACTAGGGTCCTTAAGAATTATGCTAAATC
TACTTTGTCTGTGCTCTGTCAATGGAACAACAAAGCCCAGATGACAGCACATCTGTTTACAGCATAGTTTACTGAATACTTTAAGGC
CACTGTTGAGACCTACTGTCCCCCAAAAAAGATTCCTCTCAACATATTACTGCTCATTGACAATGCACCTGGTTACTCAAGAGCTCT
GGTGGAGATGTACAAGGAAATGAATGTTGTTTTCATGCCTGCTAACACAGCATCCATTCTGCAGGCCCCAGATCAAGGAGTAATTCT
GACTTTCAAATTATTTAAGATATACATTTCATCAGGTTATAGCTGCTATTAGATAGTAATTCCTCTGATGGATCTGGGCAAAGTAAA
TTGAAAACCTTCTGGAAATGATTCACCAGTTTAGATATCATCAAGAACATTTGTGATTCATGGGAGGAGGTTGAAATATACACATTA
GTAGGAGTTTGGAAGAAATTGATTCCAACCCTCATGGATGACTTTGAGGGGTTCAAGACTTCAGTGGAGGAATCACTGAAAACGTGG
TGAAAATAACAATAGAGCTCGAATTAGAAGTGGAGCCTTGATGATGTAACTGAATTGCTGCACTCTCATGATCAAACTGGAATGAAT
GAGAGTTGCTTCTTACAACTGAACAATGTGTTTTGTTTGTTTGTTTGTTTTTGAACAATGGAATCTGCTTCTGGTGAAGATGCCATG
AACATTGTTGAAATAACAAGCAAGGATTTAGAATATTCCATAAACATAGTTGATAAAACAGTGGCAGTGTTTGAGAAGACTTACTCC
AATTTTTGAGAGATGTTGTCTTGTGGGTAAAATGCCATCAACCAGCATCACATGCTATACAGAGGGCTCTCTCATGAAAGGAAGAGT
CAATAAGTGCAACAAACTTCATTGTTGTCTTATTTTCAGAAATTGCCACAGCCATCCCAGCCTTCAGCAACCACCAACATTGAGACA
GGAAATGATTTGCAGCAAAAAGATTATGACTCATTGAAGGGTCAGATAATCATTAGCATTTTTTGGCAACAAAGTACTTTTAAATTA
AGGTATGGACATTTAGGGGTTTTTTATTTTTAGGTATAATGCTATTAACAGTCTAGAGTATAGTATAAGCTGTTATTTCAGTAGAAA
AACAAAAAATCTGTGTGACTTGCTTCACTGAGCTATTTGCTTTATTGCAGTGGTCTGGAACCAAACCCATATCGCTGAGATATGCTT
GCATAGGGGGTGTGGTAAAGACCAAAGTTTATTGTTTTTAACATAGCTATCTAACTGTTCTAACACCATTTGTTTAAAACATCCTTT
TTTTACATTGAATTATCTTAACAACTTTGGCAAAAATCAAGTGACCACCTATACATAAATCTATTTCTGAATTTTTCTGTTTCATCA
ATCTGTTTCTATTTTTATACCAATAACACATTGTCTTGATTGCTGTAGCTGTATAAGGCTTGAAATCAGGTAGATTAATTTTTCCGA
CTTTGTTCTTTTTCAAAATTCCTTCATTCTATGTCTTTTTCATTTCTGTATAAATTTTAGAATGTAGCCTGTTCCTTTCTACCAAAA
AAATCCTGTTGAGATTGTATTGAATCTACTGGGGAATCTGGAGGAGAATTGATATATTAAGTCTTCCAATTCATGAACATGGTATAT
GTCTCCATTTATTTAGCTCTTTAATTTCGTTCAGCAATGTGTTTTAGTTTTCAGTGTATAGTTCTTACAAAATTTTCCCTAAGCATT
TCATATTTTTGATATTTTAAATATTTCAATTTCTAAATATTTCTAGATATAGGAATATAATGATTTCTGTATACTGATCTGTATTCT
AGAGGCTTGCTGACCTTATTTAGATTTCATAGATTTTCTACATAGACAATCATGTTGTCTGTGAATAATGACTTCTTCCTTTTCAGT
CTGTATGCCTTTTGTTTATTACTGGCTGAAACCTTCAGTGAAATGTCAAATAGAGCTAGTAAGAGCAGAAATACTTGACTTGTTCAT
GATCTTAGGAAGAAAGCATTTAGCCTTTCACCATTAAATATGATGTTAGCTGTAGGTTTTTTATTGTAGGTCGTCTTTATTAGATTT
AGGACTTTGCCTTTTATTCCTATTTGTGAGGGTTTTTTTTTTTCCTAAATTGGAAATGGATATTAAATTTTTCAAGTGCTTTTTCTC
CATGTATTGACTATGTGGCTGATTTGATTTTTTGGGCCTATTAATTGATGCATTATTATCAACTTTTGAATCTAAAAATCACCTTAT
TCCTGGGATAAAGCCTACTTAGTGATGAAATATTACCATGTTTATGTATTGTTGGATTTGCTAAAACTTAATTTTTGCATCTATATT
CATGAGGGATGTTGGTCTCTCTCTATATTTTGCCTTGTATAATGAGGAAGCATTCCACCTCTTTATCTTCAATTATCTGGACAAGTT
TGCATAAAATTGGTATTACTTCTTCTTAAAGTTTGAAAGAATTTGCCAGTAAAGCTATCTGGGCCTGGTGTATTTTCTTTTTAAGAA
GTTTTAACTACAAATTCAATTTTATAGATATAGAAAAATTCAATTTATATATTTCTTTTTGGTTGAGCTTGTACCTTTCAAATAATT
CATCTATTTCATTAATCTAATGTATTGATATAGTTCATATTTTATTAATATCTGTAGAATATGTAGTGATATCACCTCATTCTGATA
TTGGCAAATTATCTTTTTTCCTGATAATCTGGCTAGAAGTTTTATTGATATTTTCAAAGAACCAGCTTTTGGTTTCATTGATTTTTT
TTCTGTTTTTGTTTTATATTTCACTGGCTAGATCTTTATTGTTTCCTTTATTCTTCCTACACTGTGTTTTTGTTCGTGTTCTGTTTT
CATTAAGGTGGAAGCTGATGCCATTAATTTGAGATCTTTTTGTTAAACAGGCATTTAGAGCTATAAATTTATCTTAGGTGTCACATT
AGCTGCATTTTTTTTTTAATAGACAGGGCCTCAGTCACCCAGGCTCCAGTGCAGTGGTGTGATCATGGCTTACTGCAGCCTTGAATT
CCTGGGCTCAGACGATCCTCCTGCTTCAGCCTCCCAAGTAGCTGGGACAACACGTATGAGCTACCACACTTGGCTAATTTTTTATTT
TTAAATTTTTTGTAGAGATGGGGGTCACACTATGTTGCTCTGGCTTGTCTCAAACTCCTGGGTTCAAGTGAGCCTCCCACCTCAGCC
TCCCAAAATGCTGGGATTACAGGTGTGAACCACCATGCCAGGCCCACAAAATTTGATACATTGTGTTTTCCCTTTAGATTTCTTTTG
TGATTATGTGTTATTTAGAAGTGTGTTATTTAGTTTCCAAATATTCTGGGAATATTTCCTATACCTTATACCTTTCTAACTTCTAAT
TTAATTACATCATAACCAGAAAACATGACTAGAATACTTTTAAATTTATTTAGACATTTTTTCTTTCAGTACTTTGAAGGTATTTCA
CCACCCCCCCTTTTTTTTTATTTTTTTTTTATTTTTTGGAGACAGTCTTACTCTGTTGCCCAGGCTGGAGTGCAGTGGCACAATCTC
AGCCTCCCAGGTTCAAATGATTCTTGTGCCTCAGTCTCCCTAGTAGCTGGGATTACAGGTGCACACCATGATGCCCAGCTACTTTTT
GTATTTTTAGTAGAGACAGGGTTTTTCCATGTTGGCCAGACTGGTCTCAAACTCCTGGCTTCAAGTAATCTGCCTGCCCCAGCCTCC
CAAAGTGCTGGGATTATAGGCATGAGCCACCGGGGGTGCCCTGCCCACTATGGTTCTTCCTTGTTCCATTGAATGTTTGTATTCTTT
TTTCTCTGGGTGCTTTTAAGATATTCTCTTATTACTTTTAAGTAATTTGATGTTCATATTGCCTTGATGGCATTTTATTCATTTTTC
TTATTCCTGAAGTTTGATGATCATCTTGGATCTGAGGATTTGGAGTTTTCATCTAACATGGAAAAAATATTAGCCATTGTCTTATCA
AAAATGTTTTTCTCTCCACTATCTTTCTTGGCTTTCAGGAATTCGATTACACATGTATTAGGCCACTTGAAGTTGTCTCAAAGCTTA
TTTTTTTTCTATTTCTTTTTGGATAGCTTTATTTCTATATCTTCAAGTTTACTGTTTTTTCCTGCTATATTCAATGGCTATATTATA
GCCATTCATCCAACCTAGTCTATTTTTATATCAAACTTTGTAGTTTTCTTCTATAAATTAACTTGGGTTTTTAAAATGTTCTGTATC
TATCTACACTTCTCAGTCTTGTAGCTTCTTGAACATATAGAATATAATTATAACTGTTTTCATGTCTTTTCCCACTATTTATGTAAT
ATGTATAATTTCTGGGTAGGCGTCAATTGATCTTTTCTCTCATAGGTCATATTTTTCTCTTTTACTGAATGTCTGGCAATTTTTTAT
TAAATGCCAGACATTTAGATGCTGAATATTTTGTATTCCCATAAATATTCTTGAACTTTATTGTGAAATACAGTTAGATCATTTGGA
AAAAGTTTGATGCTTTCATGTGTTGCTTTTAAATTTTCTTAGATAGGGCCAGGGCAGCATTTATTCTAGGGCTAATGTTTTCAAACT
TGTGAGGTGAAGCCTTCTATGTATTCTACCTGGTAGCACATGAATTATGAGATTTTTAGCTCTAGGGACCAGGAATGATTCCTGGCT
CTTTGTGAGCGCTGTGTTCTCTAATCTTTTTAGGTGGTTCTTTCCCCAGCCTTGAGTAGTTCCTTCATATAAATACACTGATCAGTA
CTCAGCTAAAGCCTTAAGAGACCCCTCTATAAATCTCTTTAGTGTGCATTCTCTTTGCAGCTCTCTCTGGGACTCTGCTCTGTAAAC
TCTAGTTTCTTAACTTTGCCTTACTCCTGTGCTCCATCACCTCAACTCAGGGACATCACTGGGCTCTGTCTGAGCTCCTGCTCCCTG
CAACTGATACCTGGATATTTGCTTTGGGTGGTAAGCTGAGGCAATTATAAAATTTACCTCATTTGCTTTCCTTCTTTCAGAAATCAC
TGTCCATTGCCTAATATATAAATCTTTAAACCCATGGTTTCACATATTTTTTGTTCTTTTAGTTGTTTTTGGTGGAATGGTAAATCT
GGTCCCTGTTACTATATCTTATCTAGGTGGGCCATAATAATTTTTAAATATGCATATTTAAATCTTCCCAAATTTGGGAAAATTTCT
TGGCAGATATGAATAGGGAGGGCATAAGTGTGAATTGTAGCAAAATCAAATTTTTGTTTAGAAATTTGAAGTTTAGTGTTTTTGTAC
TCTCAATTTTCTATTTTAAAAAAGGTGGTAAATGCCCTGAAAACCAGGTATAATATCATCCATACCCTTATTTCTGGACTCAGAGGG
ATTTTGAGGCCTATAAATGTTATGACTTACCCAATTACATAGCTAACAAGCAGCAGATTTGAAACCCAGAATCAGGTCTACAGAAGC
TCAGTCCCAGGTACTCTCCAATCTTCTGACACATACAGTCGATCCCCATTATTTGCAGATTGTGTTTATAAAATAATCTACATGATA
AAATTTGTAACCCAAAAGTCAGTACTTACAGGAGCTTTCGCTTTTGCTTTCATGGACACTGGCAGGGTGGTGAAAAATACTGGACAC
ACATATTCCCAGCTCTCACATTGTTAAGTTAGTTAGGTTTTTTTTTACTTTTTGCAGTTTATGTAGTGCCCTGTTTTTCACGTTTCT
GTGCTTTTCATTGGTGATTTCATTGTTTAAAATGGCTCTGAACTGGGTGGGGTGTCGCCTCACCTAGGAAGCACAAGGGTGAGGTGA
TTTCCCTTTCCTAGTCAAGGGAAGCCATGACAGACTGTACCTGGAAAAACAGGACACTCCCACCCAAATACTGCACTTTTTCCAAGA
TCTTAGCAACTGGCAGAGAAGGAGATTCTCTCCCGTGCCTGGCTTGGCAGGTCCCGTGCCCACAGAGCCTTGTTCACTGTTAGCACA
GCAGTCTGAGATCAAATTGCAAGGTGGCAGCCTGGCTAGGAGAGGGGCATCCACCATTCCTGAGGCTTCAGTAGGTAAAGCGGCTGG
GAAGCTCAAATGGGGTGGAGGCTACAGCAGCTCAACAAGGCCTACTCCCTCTATAGACTCCATTTCTATGGACAGGGCATAGCTCAA
CAAAAGGTAGCAGATAACTTCTGCAGACTTAAACATCCCTGTCTGACAGCTCTGGAGAGAGCAGCGGTTCTCCCAGCATGGCATTTC
ACCTCTGAGAACAGACACACTGCCTCCTCAAGTGGGTCCCAAACCCCCATGTAGCCTAATTGGGAGACACCTCCCAGTAGGGGCTGA
CTGATACTTCATATAGGTGGGTAGCACTCTGGGACAAAGCTCCCAGAGGAAGGATCAGGCAGCAATATTTGCTGTTCTGCAATATTT
GCTGTTCTGCAGCCTCCACTGGTGATACCCAGGCAAACAGGGTCTGGAGTGGACCTCCAGCAAACTCCAACAGACCTGCAGCTGAGG
GACCTGTTAGAAGGAAAACTAACAAACAGAAAGGAATAGCATCAACATCAGCAAAAAGGACATCTACACCAAAACCCCAACTGTAGG
TCACCAACATCAAAGACCAAAGGTGGAGAGAAACCAGAGCAGAAAAGCTGAAAATTCTAAAAACCAGAATGCCTCTTCTCCTCCAAA
GGATTGCAGCTCCTTGCCAGCAACGGAACAAAGCTGGATGGAGAATGACTTTGACGAGTTGACAGAAGCAGGCTTCAGAAGGTTGGT
AATAACAAACTTCTCTGAGCTAAAGGAGCACATTTGAACCCAACGCAAGGAAGCTAAAAACCTTGAAAAAGGGTTAGATGAATGGCT
AACTAGAATAAACAGTGTAGAGAAGACCTTAAATGACCTGATAGAGCTGAAAACCATGGCAGGAGAACTTCGTGATGCATGCACAAG
CTTCAGTAGCTGATTCAATCAAGTGGAAGAAAGTGTATCAGTGATTGAAGATCAAATTAATGAAATAAAGCAAGGAGAAAAAAAGAG
TAAAAAGAAACAAAGCCTCCAAGAAATATGGGACTATGTGAAAAGACCAAATCTATGTCTAATTGGTGTACCTGAAAGTGACGGGGA
GAATGAAACCAAGTTGGAAAACACTCTTCAGGATATTATCCAGGAGAACTTCCCCAATCTAGCAAGGCAGGCCAACATTCAAATTCA
GGAAATACAGAGACCACCACAAAGATACTCCTCGAGAAGAGCAACCCCAAGACACATAATTGTCAGATTCACCAAGGTTGAAATGAA
GGGAAAAATGTTAAGGGCAGCCAGAGAGAAAGGTCGGGTTACCCACAAAGGGAAGCCCATCAGACTAACAGTGGATCTCTCTACAGA
AACCCTACAAGCCAGAAGAGAGTGGGGGCCAATATTCAACGTTCTTAAAGAAAAGAATTTACAACCCAGAATTTCATATCCAGCCAA
ACTAAGCTTCATAAGTGAAGGAGAAATAAAATCCTTTACAGACAAGCAAATGCTGAGAGATTTTGTCACCACCAGGCCTGCCTTACA
AGAGCTCTTGAACGAAGCACTAAACATGGAAAGGAACAACCAGTACCAGCCACTGCAAAAACATGCCAAATTGTAAAGACCATCGAT
GCTAGGAAGAAACTATATCAATTAACAGGCAAAATAACCAGCTAACATCATAATGACAGGATCAAATTCACACATAACAATATTAAC
CTTAAATGTAAATGGGCTAAATGCCCCAATTAAAAGACACAGACTGGCAAATTGGATAAAGAGTCAAGACCCATCAGTGTGCTGTTG
GGAGACCCATCTCACGTGCAGAGAAACACATAGACTCAAAATAAAAGGATGGAGGAAGATCTACCAAGCAAATGGAAAGCAAAAAAA
GCAGGGATCGCAATCCTAGTCTCTGATAAAACAGACTTTAAACCAACAAAGATGAAAAGAGACAAAGGAGGCCATTACATAATGGTA
AAGGGATCAATTCAACAAGAAGAGCTAACTATCCTAAATATATATGCACCTAATACAGGAGCACCCAGATTCATAAAGCAAGTCCTT
AGAGACCTACAAAGACTTAGACTCCCACACAATAATCATGGGAGACTTTAACACCCCACTGTCAATATTAGACATATCAATGAGACA
GAAGGTTAACAAGGATATCCAGGACTTGAACTCAGCTCTGCACCAATCAGACCTAATAGACATCTACAGAACTCTCCACCCCAAATC
AACAGAATATACATTCTTCTCAGTGCCACATAGCACTTATGCCAAAATTGACCATATAATTGGAAGTTAAGCACTCCTCAGCAAATG
TAAAAGAACAGAAATCACAACAAACTGTCTCTCAGACCACAGTGCAATCAAATTAGAACTCAGGACAAAAACTCACTCAAAACCACA
CAACTACATGGAAACTGAACAAGCTGCTCCTGAATGACTACTGGATAAATAACGAAATGAAGGCAGAAAATAAAGATGTTTTTTGAA
ACCAATGAGAACAAACACACAACGTACCAGAATCTCTGGGACACATTTAAAGCAGTGTGTAGAGGGAAATTTATAGCATTAAATGCC
CACATGAGAAAGCAGGAAAGATCTAAAATTGACACCCTAACATCACAATGAAAAGAACTAGAGAAGAAAGAGCAAACACATTCAAAA
GCTAGCAGAAGGCAGGAAATAACTAAGATCAGAACAGAACTGAAGGAGATGATAGAGACACAAAAAACCCCTCAAAAAAACAATGAA
TCCAGGAGCTGGTTTTTTGAAAAGATCAACAAAATTGATAGACTGCTAGCAAGACTAATAAAGAAGAAAAGAGAGAAGAATCAAATA
GACGCAATAAAAAAATGATAAAGGGAATATCACCACTGGTCCCACAGAAATACAAACTACAATCAGAGAATACTATAAACACCTCTA
CACAAATAAACTAGAAAATCTAGAAGAAATGGATAAATTCCTGGACACATACACCCTTCCCAAGACTGAACCAGGAAGAAGTTGAAT
CTCTGAATAGACCAATAACAGGCTCTGAAATTGAGGCAATAATTAATAGCCTACCAACAAAAAAAAGTCCAGGATCAGATGGATTCA
CAGCTGAATTCTACCAGAGGTACAAGAAGAGCTGGTATCATTCCTTCTGAAACTATTCCAATCAATAGAAAAAGAGGGAATCCTCCC
TAACTCATTTTATGAGGCCAGCTTCATCCTGATGCCAAAGTCTGGCAGAGACACAACAAAAACAGAGAATTTTAGACCAGTATCCTT
GATGAACATCGATGTGAAAATCCTCAGTAAAATACTGGCAAACTGAATCCAGCAGCACATCAAAAAGCTTATCCAACACAATCAAGT
TGGCTTCATCCCTGGGATGCAAGGCTGGTTCAACATACACAAATCAATAAACATAATCCATCACATAAGAAAAACCAATGACAAAAA
TCACATGATTATCTCAATAGATGCAGAAAAGGCCTTTGACAAAATTCAGCCCTTCATGCTAAAAACTCTCAATAAAGTAGGTATTGA
TGGAACATATCTCAAAATAATAAGAGCTATTTATGATAAACCCACAGCCAATATCATATTGAATGGGTAAAAACTGGAAGTCTTCTC
TTTGAAACCGGAGAAGACAAGGATGCCCTCTCTCACCACTCCTATTCAACACAGTGTTGGAAAAGTTCTGGCCAGGGCAATCAGGCA
AGAGAAAGAAATAAAGGGTATTCAATTAGGAAAAGAGGAAGTCAAATTGTCCCTGTTTGCACATGACATGATTGTATATTTAGAAAA
CCCTATTGTCTCAGCCCAAAATCTCCTTAAGCTGATAAGCAACTTCAGCAAAATCTCAGGGTACAAAATCAATGTGCAAAAATTACA
AGCGTTCCTATACACCAAAACAAAGAGAGCCAAAACATGAGTGAAGTCCCATTCACAATTGCTGCAAAGAGAATAAAATACCTAGGA
ATCCAACTTACAAGGGATGTGAAGGACCTCTTCAAGGAGAACTATAAACCACTGCTCAACGAAGTAACGGAGGACACAAACAAATGG
AAGAACATTCCATGCTCGTGGACAGGAAGAATCAATGTCGTGAAAATGGCCATACTGCCCAAGGTAATTCATAGATTCAATGCCATC
CCCTTCAAGCTACCAATGACCTTCTTCACAGAATTAGAAAAAACTGTAAAGTTCATATGGAACCAAAAAACAGCCCGCATTGCCAAG
ACAATCCTAAGCAAAAAGAACAATGCTGGAGGTGTCACACTACCTGACTTCAAACTATACTACAAGGCTACAGTAACCAAAACAGCA
TGGTACTGGTACCAAAACAGATATATAGACCAATGGAACAGAACAGAGACCTCAGAAATAACACCACACATGTACAATCATGTGATC
TTTGACAAACCGGAGAAAAACAAGCAATGGGGAAAGGATTCCCTATTTAATAAATGGTGCTGGGAAAACTGGCTAGGCATATGTAGA
AAGCTGAAACTGGATCCATTCCTTACACCTTACACAAAAATTAATTCAAGATGGATTAAAGACTTAAATGTTAGACCTAAAACCATA
AAATCCCTAGAAGAAAACCTAGGCAATACCATTCAGAAGACAGGCGTGGGCAAGGACTTCATGACTAAAACACCAAAAGCAATGGCA
ACAAAAGACAAACTTGACAAATGGGATCTAATTAAACTAAAGAGCTTCTGCACAGCAAAAGAAACTACCATCAGAGTGAACAGGCAA
CATACAGAATGGAAGAACATTTCTGCAATCTACCCATCTGACAAGGGCTAATATCCAGAATCTACAAAGAACTCAAACAAATTTAAC
AAGAAAAAAACGACCCCATCAAAAAGTGGGCAACAGATATGAACAGACACTTCTCAAAAGACATTTATGCAGCTAACAGACACATCA
ACATGATCATCACTGGTCATCAGAGAAATGCAAATCAAAACCACAATGAGATACCATCTCACACCAGTTAGAAGGGTGATCATTAAA
AAGTCAGGAAACAACAGTTGCAGGAGAGGATGTGGAGAAATAGGAACACTTTTACACTGTTGGTGGGAGTGTAAATTAGTTCAACCA
TTGTGGAAGACAGTGTGGTGATTCCTCAAGGATACAGAACCTGAAAGACCATTTGACCCAGCAATTCCATTACTGGGTATATACCCA
AAGGATTATAAACCATGCTACTATAAAGACACATGCACACGTATGTTTATTGTGGCACTATTCACAAGAGCAAAGACTTGGAACCAA
CCAAAATGTCCATCAATGATAGACTGGATTAAGAAAATGTGGCACATATACACCATGGAATATTATGCAGCCATAAAAAAGGATGAG
TCCATGTCCTTTGCAGGGACATGGATGAAGCTGGAAACCATCATTCTCAGCAAACTATCACAAGGACATAACACCAAACACTGCATG
TTCTCACTCATAGGTGGGAATTGAACAATGAGAACACTTGGTCACAGGGCGGGGAACATCACACACCAGAGCCTGTCAGGGGGTGGG
GGTCTGGGGGAGGATAGCATTAGGACAAATACCTAATGTAAATGACGAGTTGATGAGTACAGCAAATCAACATGACACATGTATACC
TACGTATCAAACCTGCACATTAAGTTCTAGGGTACATGTGCACAAAGTATTAAAAAAAACCACACAGGGTCAAACCTAAAAGATGAC
CCATGATAAAAAAAAAAATGGCTTTGAACTATAGTGCTGAAGTGCTATCTAGAATTCCTAAGCACAGGAAGGCTGTGATGTGTGCCT
TACAGGGAAAAATTGTGGTTTGGGACACAAATTAAAAGTAATGTCCAAAACTGCGATTATTTTCCACCAACCTAAATATGTGTGTTT
GATAAGCTTTGTTCAGGAAGTTCTAGTGCTGTTAGATGTGAGTTAAATGTTAGAATGAACAATATATGTTAAATAAGATATCTTTGA
ACAGCAACACACAAAAAGCAAGGTTATGTATTGATCAGTTGGGGAAAATATGACTAGAAGCTCACAGAAACCTAACCCTGTCTTTCC
TCTAGGAGCAATGGTTCAGCATTCATTAATTCAGTGTTTGGGGCAACTTTATAGAGCATAAGTGCTACAAATGATAATTGACTATAT
TTAGTATAAATTTCTACAGAGAAATCATGAATTAAGAACTTAAAGGATGCTTTAGCCCTTTCTTACCTTATGGGAAGATACATGTGG
AATGATGTGGATGATACTTTGGGCAGTTTAACTCATATCTAGCCATGTTCCAGTCAACCTTAGTATGTGATACAAAATGAGCTTTAT
TCCTGCTAATCCAGGGTCTGAAGTTGTATTTTCATTTTTTTTTCCTGTCCTGGCAGGTACTCATGCCCGACTGTCTACCAGCCACAC
AGACTTTGAGGAGAGGGCGTATGTCGTCTTGATCCGCATCAATGATGGGGGTCGGCCACCCTTGGAAGGCATTGTTTCTTTACCAGG
TAGATATTCTGCTATAGGCAACTCAGGCTAGTATCTGTCACCATGGGCCGTTTGAGGAGATTCTTGTCTTTGATATGGGATTCTGCA
ACTATTCACTTGAGAATTTATCCTACCAAATACTGTCCTGCTTTTCAGTTACATTCTGCAGTTGTGTGGAAGGAAGTTGTTTCCGGC
CAGCAGGTCACCAGACTGGGATACCCACTGTGGGCATGGCAGTTGGTATACTGCTGACCACCCTTCTGGTGATTGGTGAGTGTAATT
TCTTAATTCAAAGAGTCTTATCCTGGGCCTCAGAAATGTGAGCTCATCTCAGGGCCTGTCTTCCTGGACTGGTCTGGTGGCTGCTCA
AATGGCAACTCAGGCAATAGCCCAGAAGGTAGATCACCAGCAAATTACATCAGACTTTCTTGATTGGAGTTACAGCTTTAGTACTAC
CCTCTGTCTCTATTGCCTTATTAGTTTAGAAATATAAGGGGCTTTATGGTCATCAATTGTCATATATTATGCCTAGTTACAAAGCTT
TTGTGATAAAGAGGGAAGAAATGCAGTAGCTGTGCCATGTGTCAGGCACTGTGCTTGATGGGGAAAGAGTGACATACACAGATCCTA
CACCAGAGTTTATGGTCTCACCTGCCATTGTTGTTACAGGCAGAAGTTAGCCTTCCAGTGGCGATGCTGAGATACCTGAAATGTGTA
GTGCAGAACTTCCCTATGCATAGAACCCTGATTCTTTACTACCCAGAATAACCCATTGCATCATGACCACAGCCTCTGCCATAGCCC
CTAGCTTGGCTAGACAGTACAGTCCCCTGATCTAGCTGTCTTTTTCTTTTTTGCAAACAACTCAGTGCCTATTCCAAGTGTTTCCTT
ACCCAAAATGCTTGAGAGCAGAAGTGTTTCAGATTTTGGGTTTTTTCCAGATTTTTGAACATTTGCAAATACACGATGAGATATCTT
GGGGATGAGACCCAAGTCTCAACACAAAATTCACGTGATTCATAAACATTTTATACACGTAGTCTGAAGGTATTTCATACAATATTT
TAAATAATATTGTGTATGAAACAAAGTTTTGCCTGCAACCTGTCACATGAGGTCAGGTGTAGAATTTTATGCTTGTGACATCAAGTT
GGTACCCCAAAAGTTTCAGATTTGAGATTTTCAGATTAAGGATACTCAACCTATATAGTAATTGCCCTCTTTCAACTGTTGTCCCCA
CCACAAGTTAAACATTAGGAATAGATACTTCATTATTTCATGTGTACCTGAGTGAAGGGGTTTATGGTTTCATATCCTTTTTCTTTT
AAAAGAAGGACTAGGATTATTCTAAGTATTATTGATTGTTCTCTCAGCATCTGATCTTTTCTCCCATGATCCAGTGTGTCACACTGG
ACTCTGATTCATGCATCTAAAGGGGGTCTCACTTCTTTATACCCTTCAGTCTGGACTCCCTATGGCATGGGAGGGCCTTCATGTCAT
GGCCACTTCCCATCCTAATGTGTGCATGGTGATCATCTTTCCTCCCTGAGAACCACTATCTACTGCACACTTTTCATGGTAATTGAG
CTGTCTTTTTGAGGTCCCACTCACTTACATAGGTCCCTTCTGCCTCTGTGCCTTTTCTTATTCTATTTGCCCTCCCACCTTGAAAGG
ATTTTTGAATTTACAAATCAGATAGCAATTTTCTTAACGTATCAATTTAATTCCATAATCTTACCACAAGTTAAAGACTGCTTGAGT
TTGGGGACATCCAGAAATAGAAGTAATAGGATTTTACAGTTTCATGACCTTATTTTCAAGATAGGGTTGGAATTATAAGTTTCTGAG
CATCCAGACTTTTCTGCCATGATTCCTCCTATAAACTTACTAGCAGGTTAATTTCCTTAAAACAACTCCTTCTTCATAGTGGACTCT
GACAGTACACAATCAGTGCTGAGCTGCACAGTGTAATTTTGATGTATCAGGTGGGAGAAACCAAGAAGGTTAAGTAGTCTACCCAGG
GTCCCACAACAACATAGGCAGGATCCAGGAGGAAGGCGCCCAAGGAAATGCGATAGTTGGTATGGACACGATGAAAATCAGCTTCAG
GGAGGTGAACAGGTCAGAGAACAGCTTTCTAGTTGTGCTAATTGGATTGTTTTCTCTAGGAAAGGGGAAGAATAGAATCAATATTAA
GTATATATAAAGTTCAGGAACTTTGTCAGGTGCTTATACACTCTATAGTGTGGGACATATACACTCCATAGTGTGGAACATTAGGTC
CTATATTCTTACAGCGAGATCCAGTGAGAGCCACCAAGTCAGCTATGATTTGGGACTTGATACATACATACAACAGGATAAATGATA
AGCTCATTTCTCCAAGAAGGCCAACGACTAGAGTCCCTTATTCACTAGGACAGTGAAATTTTGTTGAATCACATACAAAAATGAAGT
TGAGTCATTTTTTTCCTACCACAATACTACTTTATAACTGAATGTGACAGTACATTTTAAAAGGTCCCATTTATTTGACCCTGGGTT
TTTTCTTTTAGGTATAATTTTAGCAGTTGTGTTTATCCGCATAAAGAAGGATAAAGGCAAAGATAATGTTGAAAGTGCTCAAGCATC
TGAAGTCAAACCTCTGAGAAGCTGAATTTGAAAAGGAATGTTTGAATTTATATAGCAAGTGCTATTTCAGCAACAACCATCTCATCC
TATTACTTTTCATCTAACGTGCATTATAATTTTTTAAACAGATATTCCCTCTTGTCCTTTAATATTTGCTAAATATTTCTTTTTTGA
GGTGGAGTCTTGCTCTGTCGCCCAGGCTGGAGTACAGTGGTGTGATCCCAGCTCACTGCAACCTCCGCCTCCTGGGTTCACATGATT
CTCCTGCCTCAGCTTCCTAAGTAGCTGGGTTTACAGGCACCCACCACCATGCCCAGCTAATTTTTGTATTTTTAATAGAGACGGGGT
TTCGCCATTTGGCCAGGCTGGTCTTGAACTCCTGACGTCAAGTGATCTGCCTGCCTTGGTCTCCCAATACAGGCATGAACCACTGCA
CCCACCTACTTAGATATTTCATGTGCTATAGACATTAGAGAGATTTTTCATTTTTCCATGACATTTTTCCTCTCTGCAAATGGCTTA
GCTACTTGTGTTTTTCCCTTTTGGGGCAAGACAGACTCATTAAATATTCTGTACATTTTTTCTTTATCAAGGAGATATATCAGTGTT
GTCTCATAGAACTGCCTGGATTCCATTTATGTTTTTTCTGATTCCATCCTGTGTCCCCTTCATCCTTGACTCCTTTGGTATTTCACT
GAATTTCAAACATTTGTCAGAGAAGAAAAACGTGAGGACTCAGGAAAAATAAATAAATAAAAGAACAGCCTTTTCCCTTAGTATTAA
CAGAAATGTTTCTGTGTCATTAACCATCTTTAATCAATGTGACATGTTGCTCTTTGGCTGAAATTCTTCAACTTGGAAATGACACAG
ACCCACAGAAGGTGTTCAAACACAACCTACTCTGCAAACCTTGGTAAAGGAACCAGTCAGCTGGCCAGATTTCCTCACTACCTGCCA
TGCATACATGCTGCGCATGTTTTCTTCATTCGTATGTTAGTAAAGTTTTGGTTATTATATATTTAACATGTGGAAGAAAACAAGACA
TGAAAAGAGTGGTGACAAATCAAGAATAAACACTGGTTGTAGTCAGTTTTGTTTGTTGATCCGCTCTGCCTCTGTTTTTATTTGTGG
AGGGGAAGGACAGAATAACAATTGTTATTTTTTGTGTATAAAAATTGCCTCCCCAGCAGCCCTAGTCAGGGGCTTATAGATAAAACT
CCCATCTC
HUMAN mRNA SEQUENCE hR28-010.1 (Seq ID No: 61)
GGAAGAGGGAGTGTTCCCGGGGGAGATACTCCAGTCGTAGCAAGAGTCTCGACCACTGAATGGAAGAAAAGGACTTTTAACCACCAT
TTTGTGACTTACAGAAAGGAATTTGAATAAAGAAAACTATGATACTTCAGGCCCATCTTCACTCCCTGTGTCTTCTTATGCTTTATT
TGGCAACTGGATATGGCCAAGAGGGGAAGTTTAGTGGACCCCTGAAACCCATGACATTTTCTATTTATGAAGGCCAAGAACCGAGTC
AAATTATATTCCAGTTTAAGGCCAATCCTCCTGCTGTGACTTTTGAACTAACTGGGGAGACAGACAACATATTTGTGATAGAACGGG
AGGGACTTCTGTATTACAACAGAGCCTTGGACAGGGAAACAAGATCTACTCACAATCTCCAGGTTGCAGCCCTGGACGCTAATGGAA
TTATAGTGGAGGGTCCAGTCCCTATCACCATAGAAGTGAAGGACATCAACGACAATCGACCCACGTTTCTCCAGTCAAAGTACGAAG
GCTCAGTAAGGCAGAACTCTCGCCCAGGAAAGCCCTTCTTGTATGTCAATGCCACAGACCTGGATGATCCGGCCACTCCCAATGGCC
AGCTTTATTACCAGATTGTCATCCAGCTTCCCATGATCAACAATGTCATGTACTTTCAGATCAACAACAAAACGGGAGCCATCTCTC
TTACCCGAGAGGGATCTCAGGAATTGAATCCTGCTAAGAATCCTTCCTATAATCTGGTGATCTCAGTGAAGGACATGGGAGGCCAGA
GTGAGAATTCCTTCAGTGATACCACATCTGTGGATATCATAGTGACAGAGAATATTTGGAAAGCACCAAAACCTGTGGAGATGGTGG
AAAACTCAACTGATCCTCACCCCATCAAAATCACTCAGCTGCGGTGGAATGATCCCGGTGCACAATATTCCTTAGTTGACAAAGAGA
AGCTGCCAAGATTCCCATTTTCAATTGACCAGGAAGGAGATATTTACGTGACTCAGCCCTTGGACCGAGAAGAAAAGGATGCATATG
TTTTTTATGCAGTTGCAAAGGATGAGTACGGAAAACCACTTTCATATCCGCTGGAAATTCATGTAAAAGTTAAAGATATTAATGATA
ATCCACCTACATGTCCGTCACCAGTAACCGTATTTGAGGTCCAGGAGAATGAACGACTGGGTAACAGTATCGGGACCCTTACTGCAC
ATGACAGGGATGAAGAAAATACTGCCAACAGTTTTCTAAACTACAGGATTGTGGAGCAAACTCCCAAACTTCCCATGGATGGACTCT
TCCTAATCCAAACCTATGCTGGAATGTTACAGTTAGCTAAACAGTCCTTGAAGAAGCAAGATACTCCTCAGTACAACTTAACGATAG
AGGTGTCTGACAAAGATTTCAAGACCCTTTGTTTTGTGCAAATCAACGTTATTGATATCAATGATCAGATCCCCATCTTTGAAAAAT
CAGATTATGGAAACCTGACTCTTGCTGAAGACACAAACATTGGGTCCACCATCTTAACCATCCAGGCCACTGATGCTGATGAGCCAT
TTACTGGGAGTTCTAAAATTCTGTATCATATCATAAAGGGAGACAGTGAGGGACGCCTGGGGGTTGACACAGATCCCCATACCAACA
CCGGATATGTCATAATTAAAAAGCCTCTTGATTTTGAAACACCAGCTGTTTCCAACATTGTGTTCAAAGCAGAAAATCCTGAGCCTC
TAGTGTTTGGTGTGAAGTACAATGCAAGTTCTTTTGCCAAGTTCACGCTTATTGTGACAGATGTGAATGAAGCACCTCAATTTTCCC
AACACGTATTCCAAGCGAAAGTCAGTGAGGATGTAGCTATAGGCACTAAAGTGGGCAATGTGACTGCCAAGGATCCAGAAGGTCTGG
ACATAAGCTATTCACTGAGGGGAGACACAAGAGGTTGGCTTAAAATTGACCACGTGACTGGTGAGATCTTTAGTGTGGCTCCATTGG
ACAGAGAAGCCGGAAGTCCATATCGGGTACAAGTGGTGGCCACAGAAGTAGGGGGGTCTTCCTTGAGCTCTGTGTCAGAGTTCCACC
TGATCCTTATGGATGTGAATGACAACCCTCCCAGGCTAGCCAAGGACTACACGGGCTTGTTCTTCTGCCATCCCCTCAGTGCACCTG
GAAGTCTCATTTTCGAGGCTACTGATGATGATCAGCACTTATTTCGGGGTCCCCATTTTACATTTTCCCTCGGCAGTGGAAGCTTAC
AAAACGACTGGGAAGTTTCCAAAATCAATCGTACTCATGCCCGACTGTCTACCAGGCACACAGACTTTGAGGACAGGGCGTATGTCG
TCTTGATCCGCATCAATGATGGGGGTCGGCCACCCTTGGAAGGCATTGTTTCTTTACCAGTTACATTCTGCAGTTGTGTGGAAGGAA
GTTGTTTCCGGCCAGCAGGTCACCAGACTGGGATACCCACTGTGGGCATGGCAGTTGGTATACTGCTGACCACCCTTCTGGTGATTG
GTATAATTTTAGCAGTTGTGTTTATCCGCATAAAGAAGGATAAAGCCAAAGATAATGTTGAAAGTGCTCAAGCATCTGAAGTCAAAC
CTCTGAGAAGCTGAATTTGAAAAGGAATGTTTGAATTTATATAGCAAGTGCTATTTCAGCAACAACCATCTCATCCTATTACTTTTC
ATCTAACGTGCATTATAATTTTTTAAACAGATATTCCCTCTTGTCCTTTAATATTTGCTAAATATTTCTTTTTTGAGGTGGAGTCTT
GCTCTGTCGCCCAGGCTGGAGTACAGTGGTGTGATCCCAGCTCACTGCAACCTCCGCCTCCTGGGTTCACATGATTCTCCTGCCTCA
GCTTCCTAAGTAGCTGGGTTTACAGGCACCCACCACCATGCCCAGCTAATTTTTGTATTTTTAATAGAGACGGGGTTTCGCCATTTG
GCCAGGCTGGTCTTGAACTCCTGACGTCAAGTGATCTGCCTGCCTTGGTCTCCCAATACAGGCATGAACCACTGCACCCACCTACTT
AGATATTTCATGTGCTATAGACATTAGAGAGATTTTTCATTTTTCCATGACATTTTTCCTCTCTGCAAATGCCTTAGCTACTTGTGT
TTTTCCCTTTTGGGGCAAGACAGACTCATTAAATATTCTGTACATTTTTTCTTTATCAAGGAGATATATCAGTGTTGTCTCATAGAA
CTGCCTGGATTCCATTTATGTTTTTTCTGATTCCATCCTGTGTCCCCTTCATCCTTGACTCCTTTGGTATTTCACTGAATTTCAAAC
ATTTGTCAGAGAAGAAAAACGTGAGGACTCAGGAAAAATAAATAAATAAAAGAACAGCCTTTTCCCTTAGTATTAACAGAAATGTTT
CTGTGTCATTAACCATCTTTAATCAATGTGACATGTTGCTCTTTGGCTGAAATTCTTCAACTTGGAAATGACACAGACCCACAGAAG
GTGTTCAAACACAACCTACTCTGCAAACCTTGGTAAAGGAACCAGTCAGCTGGCCAGATTTCCTCACTACCTGCCATGCATACATGC
TGCGCATGTTTTCTTCATTCGTATGTTAGTAAAGTTTTGGTTATTATATATTTAACATGTGGAAGAAAACAAGACATGAAAAGAGTG
GTGACAAATCAAGAATAAACACTGGTTGTAGTCAGTTTTGTTTGTTGATCCGCTCTGCCTCTGTTTTTATTTGTGGAGGGGAAGGAC
AGAATAACAATTGTTATTTTTTGTGTATAAAAATTGCCTCCCCAGCAGCCCTAGTCAGGGGCTTATAGATAAAACTCCCATCTC
HUMAN PROTEIN SEQUENCE: hP28-010.1 (Seq ID No: 62)
MILQAHLHSLCLLMLYLATGYGQEGKFSGPLKPMTFSIYEGQEPSQIIFQFKANPPAVTFELTGETDNIFVIEREGLLYYNRALDRE
TRSTHNLQVAALDANGIIVEGPVPITIEVKDINDNRPTFLQSKYEGSVRQNSRPGKPFLYVNATDLDDPATPNGQLYYQIVIQLPMI
NNVMYFQINNKTGAISLTREGSQELNPAKNPSYNLVISVKDMGGQSENSFSDTTSVDIIVTENIWKAPKPVEMVENSTDPHPIKITQ
VRWNDPGAQYSLVDKEKLPRFPFSIDQEGDIYVTQPLDREEKDAYVFYAVAKDEYGKPLSYPLEIHVKVKDINDNPPTCPSPVTVFE
VQENERLGNSIGTLTAHDRNEENTANSFLNYRIVEQTPKLPMDGLFLIQTYAGMLQLAKQSLKKQDTPQYNLTIEVSDKDFKTLCFV
QINVIDINDQIPIFEKSDYGNLTLAEDTNIGSTILTIQATDADEPFTGSSKILYHIIKGDSEGRLGVDTDPHTNTGYVIIKKPLDFE
TAAVSNIVFKAENPEPLVFGVKYNASSFAKFTLIVTDVNEAPQFSQHVFQAKVSEDVAIGTKVGNVTAKDPEGLDISYSLRGDTRGW
LKIDHVTGEIFSVAPLDREAGSPYRVQVVATEVGGSSLSSVSEFHLILMDVNDNPPRLAKDYTGLFPCHPLSAPGSLIFEATDDDQH
LFRGPHFTFSLGSGSLQNNWEVSKINGTHARLSTRHTDFEERAYVVLIRINDGGRPPLEGIVSLPVTFCSCVEGSCFRPAGHQTGIP
TVGMAVGILLTTLLVIGIILAVVFIRIKKDKGKDNVESAQASEVKPLRS*

TABLE 7

MOUSE GENOMIC SEQUENCE: mD28-021 (Seq ID No: 63)
AGGAAGGAGCGGAGGCCCTGGTGCCCTGCCCTCAGTGCTGACTGCCCAGCGAGAGTGACTGGTATAAGCAAGCAGAGACCCGGTGAG
TGTGCCCCAGTGTGCGGCCGGGGCTGCAGGGGTGGCTGTGGGCCGGGCTCAATGACTTGGATTTTGCTGTGAGATTCATATAGAGTT
GTGGATGGGATGTCCTGTGTGCACGTGGCTGAGAGACGCTAGGGTGGGATGAGGGAGAGAAGGGAGCTGAGCCTTGTTTAGGGAGGT
GGGAAAGACAGACCACCACCGTGAAGGGTGGGGGGTGGGACGTGAGGGAGGGGAGCTGTCTGGCTAGCGTGGGGGACAGAGGTGGGT
GGGGAGTGATGAGGTTCATCAGCTGCCGTAGGTGTGAAATAGGCTTCCAGATGACACCAGAGTCTGCCTGCTTCTCCATTCCCATGT
TTCTGTATGGAAAACAGACTGCCTGTGAACTGAGGAGTATGGGAGGAGGAGCAGGGTGCTCAGAGCGGTGGGCTGGAGCTGTAGGGA
ACAAGGTTAGGCAGTTAGCTAGGAGTCTACATGGTGGGCTTGGATGTGAGGCCGAAGGCTGTAGATGTCTTCCTAAGGGGGCTTTGG
AAGAAGGAAAGTTTCTAAAGCAACAATGTCCTTACATTCTTCTGTCACTTCCTTCGTGGAACACTGGCAAGTGGCACCTCCTGCAAA
CTAGAGCAGCAGACAGAGCACACCCCGTGGACATGATTAGATCTGCATTTAAAGCTGGGCAGTGGTGGCCCGCACTTTAATCCCAGC
ACTTAGGAAGCAGAGGCAGGTGGCTCTCTGAGTTTGAGTTTAGCCTGGTCTCCAGAGTTCCAGGACAGCCAGGGCTACACAGAGAAA
CCCTGTCTCAAAAAAACAAGCAAGCAAACAAAAATATCATTTAAAAAAGGATTGTTTTTATTTTATGTGTATGTTTGCCTACATGTA
TGTATATATTCCATGTGTATGCCTGGCACTTGTGGCGGCAGAAGAGGGGCGTTGGGTCCATTACAGAGTGTTGGGAGCCACCATGTG
GGTATTGGGAACTGAACCTGGTTCCTCAGCAGGAGCTTTAAAACACTCTTAACTGCTAAGCCATATCTCCAGCCCCAGGTTTGCATT
TTTTGTGAGCTAAAGGCACCATTGGCTTGTGTTCAGGATGAATTGGAAGGAAATGATGGAAGGCAGGGTGCCCACTGAGAGGATGGG
ACTTAGTTGGGATAGATGAGGGAGGGGCAAATTCAAACTCCCCACAAGTTGGGTGTTTAGAATGAAGAAAGGGCCTTCAAGGGTTTG
CTGACCTGGATAACAGGGTGTCCTTGAGCTTGGTGGGCCTTTCTCTGTAAGCTTGAAGACAAGACCCATGCCTCCGGCCATCACCCT
GTTCATTGGACAGTTCGAGATATGGTTAAACAGAGGAAGCCATGACAAAGATTGATCTGAGCCAGTGAAGAGGCTCAGCAGGGAAAT
GTGCTTGCTGCCTGACCGTCCGAGTTCCCTTCCCAGAACCCAGTGGTAAATGTGGAAGCAGAGAATTGACTCCACAGAGTTGTCCTC
TGACCCCTGAGTAACCGCACACAATTATAACAAATACTTTTTTTTTTGAAAAAAAGAAATGGATCTGGTCTCGATGGAGAGGAATGT
AGATGCTGTGGGTGGTCACACTTGGTGAAATGAGCAGCAGGGTTATGCGGAAGGACAGAGGGGGCTGCCATGGGCAGGAAATGTCAG
CTGGGGAGGACGGGGGTAAGGAAACCCTTCCGTCCGTCCTCAGGTGAGGGGGCTGATCTGCTTAAAAATGCAGAAGAGGCTGGGAAA
GAAGAGCCCTTTGAGCTCTGGGATGCTGCTTGTTAATGGCTTGTCTGGAGGGTGGAGATGGGGTGTGTATGGATGGGGCCCCGCTCC
TGGAGGCTGAGTTAATTTGGGGGTAGGGAGAGACGCTTTGAGAGTCTACAGACCAGGAAGGTGCTGATGACATCAGGCAAGCTAGGA
TAGGACTGCCTGTGGTTGAAGGTGTAAGTGATTCTTAGGGGACTGGGTTCTAGAGCTGGGTCAGCCTGTAGGCCAAGGGCTTGTCAT
GTGTCAGGGTTTCTCAGTTGTGGTACTGTGACCTTTCTAGACAAGGTGATACTTCTTTGTGTGTCTGGCCTGATGTTGTAAGATGTC
ACAAGTATCCAGGGATTCTTCCCATTAGACCCTAGCTAATACCACCTACCTCTATCCCCAGTCCTAACAATCAGAGATGTCTCCAGA
CATTACCTCACGTCCTGTGGCAGGGTAGAGCCACCCTGGACTGAGAACCACTGGTTTGCATTGCCTCTTGTGATTTCACTAAATCTG
TACAACCTGGAGACCTAGGGGGAGTCACTTGGAAGGTGTGTTTATGGATCACTTGAATCTGGTTCTGAAGTGGGCAAGAGGAGGCAG
GAAAGATGGTGAACACTAATCTAAGCAATCCCTCCCCACACCCCCAAAAGGCTGAGGGGTGGTAGCTGTAGAAAGGAATCCACTTTG
ATTTTTGTTTGTTTCATAACGAGGCCCTTGTTAGGTTCTTGGCTGCCACTCACAGAGATTTTGACACCTGTGGTTTCCCCTTTGACT
TCCCCAGACATACAGAAGCTGAGGGCTACAAAAAGGGCCTCAGTGTAGTTAGAGGATCCCTAACACTGGAGTGGGAGGGGATGTTGT
TCCTGGCTGTTCACATGGAGTCTGGCAGCCCGGCTGGAGGAGGTAAGACTGTCTCAGGCTCCTTCCTGCCAGCCACCTTCTCTGCGC
CGGGGTCAAGGACAGGGTTGTGATTTATTCCCATCACAATCCAGCAGTTGGGTTGGTGAGAGTTGGACCCATGCTGGTTTTCCCTTC
CCAAGTGTGACAAGGCCTCAGCAGCGGCTGCTGCAGTACCACGCGGTGAACTGTCCAGGGCATGACAAAAGGGCTCGGTTAGCTGCC
GCCACAGGTTAGAAGATGAACAGCTCACAGCTCTTCCGCCCCATGTGCAGCTTGTCGCTTAAGCCCTGGCCGAGCACACTGTAGAAC
CCGGGATGCCCCTTCTTCTGCCCATGGCCATGTTTGGTCTCCAGTCTCCTGGGAGCCAGGGTCCTGGCCTGAAGAGGAGGTGAGGGA
GGCTAGAGGAACAGGTGGTATTTGGAGACTAATTCATATTTGTGAATAATAACCCATGCCCACAGGAAACAAATCAACCTGTGTTTG
CATATTCATGAGGGGGGCCTTTAGAATGATCCCAGCTGTTTTCCATCCGGGAATCCCTGGGTCCTGGCGTCACATGGTCTCTTGGGG
GTCCTCTTTGGCCCTGGCAGGGATAGCTAGGGTGGATCTCATGGAAGTGACACGCATTTTCCATGAGACACATACATGACTCCTGTG
GCTTGGTGTTGTGCCTGCTGATCCCTCTTTCCTCAGAGCAGTGGTTCCCAGGATCTTTGGTTTCACACACAGTTCAGTTTCCAAGAG
TGCTTAGGGCCAGACAGGGGGTTAGCAGGTTTTTTTGTTTTTGTTTTTTTTTTTTTGTTTTTTTTTGCCAAGTAAGGACATTATTAA
AAAAAGAAAAAAAAACAACTGCTATCGGCTGTCCCTATCACTTCTTCATACAAGAAAGGATATTTTAGCACCAAGGACAAAATTTGA
GAATAAAGGACATTTCTTCCTAAGAAAGGGCAGTTGGCAGCTTTACAGTGGTGTAGCAAGGAAAAGAGATATATCAAAGCCATTCTG
AATGTAGAAAAATTACACTCAATTATATCCTATAATATCGTATGGCACGTAATTGTTTTCCTGAACAAGTATGTCAGAACCAGCAGT
CCTCGGTGCAATTCACAGATGAGCTTGAGTGTCCAAGGGTTTGGGCCTTGAGTCTATCTGAATTGTCCCGGGCACAAGCTCATCTGT
CCATGCTGGTGGAGTGACTGTCTGTTCCAAGCACCGCTGTGTTGGGTGGGTGCACGAAGCCATGGCAGTCCAGCAAGGCAGACTCGT
TCTGCAGGGGACCTGGAGACCAGGGTGTAGGCAGGACCAACATCTAGACTTTTAAGCCCAGGCCTGGGACTCAGGGTGCTCACCTCT
CTTCTGATGAGGGAGGAGGCCCTGGAGCTGGGAGCAGCCTTGAACTGCCTGACACCAGCATAGGCTCTGTGCCCAGAGTCCAGGGGT
GGGAGTTGCTGAGAAGGAGAGGCCCTGACTCGGGAGACTGTGTAGTTGCCAAAGCGGTTCCCTGTTCGTCCCTGGGGTCTTCTGGTG
TTTCTAATTGTCTGTGGCCTTGCGTTCAAGGACCACTGGAAGCAGCTCTCTTTGGGGGGAGATGTCTAGATTTTTCAACTGAAAAAA
GAAAAAGGTTTAACTATATCCTGTGTATGAGTGTCTGCCTGCCTGTGTGTCTGTGTACCATGTGTGTGTCTGGTGCCCACAGAGGGC
AGAGGAGGGCGTTGCATCCCCTGGAACTGGAGTTGGACAGTTGTGAGACGCTGTGTAGGTGCTGGGAAAACTGATGCAAGTCTTGGG
GCAGCCTGTGCCTTTAACTCTGAGCCAACTCTCTGACCCCTGTTTTCACCTGGTCTGCCCATTTTCTACACCGTGGAAGTGTATACA
GTGTTCTGAACACTCCCCCTGACTTACACAGGGGCTCTTGAATCCCCAAGTAAGAGCCGCCGGCCTTTGCTATGCTGGGAATTGGGC
TCAGATAACGCTTGTGGCCAGATTTTCACTTACCTGTTCTGGGAGTGAGACACTATGGTGGCTTTACTGAGAGGAGCTTTGCTCCCC
TTTAATAGTTAATATCCACATCCCCATCCCGTGCCTGTGACATGTCTGTGGACATCCCTGTGATGCCCTTATCAGGCAGCTGTACCC
GCAGATGTCTGTACCCTCTGTGTACCCCACATGATGTGTCTGCACCTGGGAGTTAACCATGCTGTGATTGCTGTACTAAGGCAGCTA
CAGGGGGATTAAGGTTCCGTAATTGTACTGGGGAATTGTGCCCCACGATGACTGTGCTGTGCATTTTTGTACGGCGTAACATCTATT
CTGTCTCATCCCTTTATGTCTGTACCAGGGAGATGTGTCCCCATGACACTGGTACTGAAGATTATGTCCATATGGTGTCTGTGTTGG
GGGTGGGGAGCGTGCCTGGGGTACCTGAGCACACAAGTTAAACTCCAGCTTTTGTGATGTCTCACCCACGAGACCTGCCTCGACCAC
TCTGCCTGACTTGTGTGTGGGGTGTCACACTTCTGAGTTGTACCCGTGGTAATGGTACCAAAGCAAGAGTCCTATCTGAATACAGTG
TTCCCATGGTAACCTGGCCAAGGCAGTTCACCACCTGGTGTCCTCATAGCCACACTAAGATAACCATGGTGCTGTGATGAACAAGCC
CAGATAAGACTTTAACAGAATTAGAACCCCTATCCTGACATGGCAGTGTGTGGCCTCACTGTGGGCTGACCCACCTCAGCTCTGTGG
TCCCTCAGAGGAACTCCCGGCCTGGAATTAGGAGTTCTGGACACTATGTCCTGTCCCTGTAGCGTTATGGCTCTTGCTGCTTATCCC
TTGCTTACAGGTTGGGTAAGCGTCTAAGACAGGGAACATCTCCCCTTCCCAATGGTCACTAGCTCCTCGCTCACTTCACAGTCCCAC
CTGCCTTACCTTTGCCCATTCACCTCATGATCGTGGCTATCCTAAGTTCAGATAAGGGTTCAAGTCCCAGGGAGGGGCAGAGCTATG
CTTGAACTCCATCCCTCTTGTTCACCTGTCCTCTCATTTTGAAAATTCTCTTAGCACTATAGGGGCCCATGGGGTCCCAAGTTGTAG
TGAGAAATTTGTGAGGTTTTTGTTTTTCAAAAATACAGAAATATAATGGGAATGTTTTGTTTAAATCCCAGGTGTGGGGTAGGGAGT
GGCTTCAGACTGTCCACAGCAGCTGACTATGATTTGCCTCGTGCTCTAGCAGGGGCGTGATTTTTGCCAGTGGCAGATAGCTTCTGC
AATTGTATGATATTTGGAATTTGGGGGTCTTTTCAGAGAGTATTCATTCGTGGAGAGGTGGGGTGGGTTGTTGGTTGGTGGCTGTTT
GTTAAGTAGTTGTGTGCAAAGAAGGAACGAGAAGAAATTAGATATCCCAACTGTGAAGATTAAACTTGCCCCAAGAAGCTCAGCACC
CCATTCAGCAGGAAGCTCTCTAGTGATATTTTGCCCCCTTCCTCCTCTACCCTTTTTCTCTCTCCTATGTTTAGAGGATGAAAGGGT
GGTAAGAAGGGCAGAAGAAAAAAACCCCACAAAATAGCAAAGGTCCAACTACATTAAATTGTGTCCGGTATCACTCCTCCATTAGAC
CCTGTTGTATTTTCAGAGCATGGCTGAAGCCTGGCCATGCACCAGTGGCCTGTATGTGGCAGTGTGAGCAGAATTGGTGGGCCCTGC
TGCATGTCACCTAAGCCCCCAGACAGGCAATGTGGCACCGGAACCTTTAGAAGCTCACCCTAGGTACATAGTGTGGCCACTTCTGCA
TGGGTTTGCAGAGTGAGAAAGTCATGTAGACAGGAGGGTGAGCAAGGTCTGGAAGGCTTCGTTTAGCCCCAAGCACAGCCCTCCCCC
ATCTGCTCTGGACACCCGGAACAAGGGAAGAGGAAACATGGATGTTGACACTCAGGCTCTAGAATTCCAATCCACAGCCGCTTTATA
GTTTGTCTTTGGGATAGGAAGGTGCCATGGGAATTAGGTAGGTTGAGAGTGACCAGGCAGGGCAGCTGTAACTTGTAGAGAGCAGTC
CTGGCGAGCCCCAACGGGTTGTGGCTTTGGCTGTGAGCCTCTGACGATGTGGAACCAACTACTGCTAAGTCTGTGTACTGGGGCTTT
TCCTGAGTGTGCTGGGGTCTGGCCACTTGCTTTCTGAGGAAGAGCAACAGGATGCCCCAGGGACTAAGATGGCCCTGGACAGTGTAA
AACCTGCCTGGCTGCTCTCTGTTCCTGCCTCTGCCTTGGTTTTGTTTGGGTTTTGTCCCAACCTGTATGTATCTTTGACTCCATTTC
CCTGCCCTCATCCCCTTAATGGTGTGAGCCGGAAACTCACATCTGGAATGGACCCGCCTCGTGTTAGTTTCTAACTAGCTTTCTTCC
CCCTCCTGGCGCTTAGAGGATGCCCTGAAGGTCTCTGCACTCTTTCCATAGGATGGTTGACCTTCAGTGGTGAGGGAGTTTGGGTAG
CCATTGCATGGTCCAGTGTGGGACTGCTGAACTGTCTGAATGGGCAGTGAGGGTGGGTGGCAGTGGGGTGGGGAAGAGCATGTGCAG
TGGGGACTCAGGGGGACACTGGCTAATTGGCTCTGCCTTTCTCTCCATTACAGGTGGATTGTCTTAGGCTGTGGCTGGCTGTGGAGC
TAGAACCCTGGATGGCTCCCGAGCCAGCCCCAGGGAGGAGGATGGTGCCCCTTGTGCCTGCTCTCGTGATGCTTGGTTTGATGGCAG
GTGCTCATGGCGACAGTAAGTCTGACTCCTCATCATACAGATACATCATGTTGAAAATCGACTTAGCTCCTAGCAGGTCTCTAAAAT
GCAATACCGGACCAGGTCCCAGGAAGAAGAAACAGGATGACCATAGATAACCCACTAGTCCTCCTGCCCTAGAGACCTGCACTGGGA
AAGCCCTCTTCTAAGGAGTACGTGCTCCTACTTCCTCTGAACATCCTGCTGAGCACCTGGGTATCCCTTAGGCTCTGCAGAGTGCTG
CATGTCTAAGCGTGACCCTGTTATATAGAGCTGGGGAGCCGTGTCTTGGATTCATGTGATCTTTGTGTGACGTGCATAGGCATGTAC
TTTGTCCACATTGCAGATCTCTGTAGAAGGCCCCGAGCAGAAGGAAGCAGGGCACCAGACATGTCTGCAGAGCCAGGGAGGATTCAG
AAGCAGTTGCCCATATCCTTCCCTGAGGCGGCCTGCAGTCCCTGGAAAATAGGGAAGGACTTGTCACTTGGCTTGCAGGAAGGAGAG
AGGGGGCAGGGAATGAAACTGAGTCTGTGTGTTTGTGTGTTGGGGGGATGATTTGTCCCCTACTCCAATCCAACTCTGAACTTTGGT
CTCTTCCCTGCTGCTGCCCTTCCCCCTGCCTGGCACTATAAGACTGGCCATAGCCCGCCCTGTCACCATGGTTACCACTGCTTGGTT
ACTGGTAGGAGGCGGAGCACAGGACAGGCTTAGCCAATCTGCAGACTGGGGGGAGGGGCAAGGTCGGAGCTGAGGTCCCTTGGGGGG
AAGGGGCTATTCCCAGCCTGTCCTGAACCCAGAGGCAGTCCAGGGCCAACCCAGGGGTCAGACCCCAGGATAGCAGGCCATAGGGAT
CCCTCTATCCCCTCTGGGTCTCTAGACAAGAGCCTTGTCCCACCAGGGCTCTATCTAAATGGCTCATTTTTTTTTCAGGAGCCAGTC
ATAGAAGAGGGTGGCCCTGGAGCACCCCTCCCTTTGTCCTCAGAGGCTGGGCCCTGGATAAGGGAGAATGGGAGGGAGAGGGCCTTT
CTCTGGAGAGTCTAGCCAGTGGAGTGCAGGGCTAGGGGTCTAGAAGAGAAGTCAGGTGGAAAACCTTAGAAAGGGGTAGTGTGAAGG
TGATCCTTAGCCCTGGCTGGGGCTGCAGAGGCAGCAGAGGGAAGTGATCTGGGGCCCAGCACCCTAGGTGTGTGACTGGCATTGACA
TGGTGAAGGACTGTTGTCACTCTTCTCAGATTGTCTCAGGGCCCTGACAGCAGGGGAGACATGCCACTGAGTTTTGAGAAACAGTGA
GGCCTAGGAAAATAACTGCCTTCTCTATCTTCCCTCTGACCCAGCACACCCCAGCCTGCGCCCGTCCGTCCCACCATCCTGCCTGCC
CCCTGGTTTTCTCTGTGACTGGCATTACTGTCGACCTGGTTACCAAGTCAAAAAGCTGGGCCTTCTGGCAGCTCCTCTCCCCCTTCC
ACACCCCCCTCTTCCCCCCGTGTGATCTGTCCCCAGGTTCTGTGGCCTCTAGCTTCTGAGTGTCTTTGGAATGCACTCTCTGCTCTC
CCTGCCCCCTTCTGCCAAGCCTTCTTCAGCTTCTGCTTGAGTTTGAGTGTGCACCAGGGAACCTTCTGATGGTTCCCTGCCTCAGTC
CCATTCCTCTGAACCCATTCTCTAGTCTACAGTGGGAATTGGGTTGCGTCTCTTCGCTCCCCAACTGCCTGGAGAGTGAGCTTTAGT
GTCTGACTCTCTGCTACCCTACCCTCCACCGGCCCCGCTGTGCTCTCTCCTTTCGGCTGCACAGAGCTCCGTGTCCAGGCATCCTTC
ATGCCTTCTGTTTGCACTCTCGCAGTCAGGTGTGATGACGTGGGCCTGTATCCCTAGCATTTGAGAGGTGGAAGCAGGAGGATTAAG
AGTTCTAGGGGATCTTGGCTCTGTAGTAAGTTGAAGACAAGCCTGGGCTACGTGACTGTCTCCAAAAACAAACAAGGTGTGGAGGGG
GGGTGTTTTCCCTCCCTTTACATGACCAGAACTGACTCAACTGTCACTGCTTCCTGTAGCAGCAGTGTGGTCATGCCCTGGTCCTCT
GCAGCCCTCATATGCTCTTGTGGCGCTTCGCCCTCAGTCTGTCTGCCCTGGCAGGCTGAGAGTTTAGACATTTCCAGCTCCGGGTCT
AGAGGGGGTTGGTGAATAAGCGAAGGGGCAGGACAGTCCTGACCACCGTAGGAGTGGGGAGTGAGAGGAGCTGCTTTAGGGCAGAGT
TCCAGGGTCTTCCTGCTGGCACCTTGGTCATTTGGTTACAGAGACCAGGCCGGGACTTGATGGTGGAAACCAGGTGAAGAGGCTGGG
ACCCTGCCTCTGCTAAGGGTGAACCCTCAGGTGCTGTGACTTGGGCACTGTCCCAGGCCAAGAGCCATCAGCTAGGGGCTGTGGTAA
GCCAACCTTAGGAGTCAGGGTCTGCCCTCCTAGCCTACCTTTAAGTTACTCTTTTTTTTTTTTTGAATCTTCCCCCATTGTGACCAG
TCTTGGACTGAACTTGCAAGACTTCTCACTAGGGGCAAGCAGCCAGACACGGACAGGGCTGCTGCAGAAGGCAGGGCGGGTGCCCCC
AGAGAATTGACGACCAGAGCAGTGAAGCGCAGAGCGGACAAATCTGTTTCTATAGGCCTCTTAACGAGACAAATGAATGGGGGCCCT
GGCCTCAGAGGGGAGTGGAGAGAAGCAGGGGAGAGAAGCAGCTGCCAAGAGCTAGTCTGGAGGCCTAGCAGTGATGCCTAGTCAGCT
CCAGTGGAAACCTGGATACGAAGGGATGCTTGCTTGCATGCACGCACCCTTCTGGAACCTCCCTCACCTCGTGTCCCCAACCCAGGA
TTTTATGCCTTCTTGCGAGGGAAGATATAGATGGATGTGGGCATAGAGGTGGTCTTGGTCCAGCCCTCAGGCAGAGGTTTCTGCTGA
CCTCATGTGGTAGGTGGTAGGAGAGAAAGAATCTGCCCAGACCCATCACAAGACAGAACCCACCGAGCTGGGCACACGTCTGTGAGC
TAACAGATCAGTGGGGGCAGGTTGGGGCAGCGCTGGCACAGGCAGCAGCCTGATTATCTTGAGAGGCTTCCTGGAAAAGAGGGCCCC
TGAAGATGAGTGGGCATTTGCTAACATAGAGGATTGCCCAATGAAGGAATGAGACTGGCAGAGTCATACCCCTGCCCTGGCCTTCTG
AGCTTACGAGAGAGCCAGAGTCCTGAAGACAGCACAGGCCAGCGATGGGATCCCCGCCCCCAAGACCAGGTGTACGTTGGGTCCTGG
TTATGACCAGGTTATTTGCAGGCATTAAAGTTGAGCAAGGTGCTTGGTAGGTGAAGGGGTCGGTGGTATGAAGCAGAGAGGCAGGAG
ACTGAAGAGTGTATGTGTGTGTGTGTGTGTGTGTTGCTTCTCCAAAGCCCCTATTTTGGAACATGGCAGGGCCCTCCCCCTCATGTT
TATGGGTGGCCCGTCTAGACCCTGCCCTCTGGAGCTTCCGGCACAAGTGCTTTCTCTGAGCCTGTTTTCCTATTGCACAACAGGGGT
AATCACTCCGACCTGTAAAGGTTAACCGAGGTCACATGGGCAAGGCACCTGGCACCTATTGAGTTCTCCCCCTGTTGCAGTGCCTCC
TGGAGCCAGGATTGCCCTTGAGTGGACAGGGCTGGGGGAGGCTGCCAGGAGCAGTCACAGGAGCTCATTAAGTCCCCAGCTCATTGA
GTCCTCAGCTCAGAGCCACTCAGGGCCTGGGAAATCACCTCTCCAGCAGCTCCCCTGCCCTCGTGGTCCTAAAGTGCTGTGTGTGCT
TCCTCCGTGTGCAGTGCTGTTTGTGTGCCTGTGTATATGTGCTTCTCAGGGGAGGCACAAAATAGCAATCAGTCTCCCCATGGAGGA
GCCTTGGAGGTGGCCCAGTTTAGCCGTCTCTGGAGTTGCATGTCTTCATGTCTCCAGGGGCTGTGCCTCCCTCTAGCCCAAGCCAGG
CCCTCTCTCTGGTAACTTCCTCTCATGTACAGGGTCAGCGGTTGACATGTAAAGCATGGCCCAGGGTATCCTACTGTGAGTAGCTGC
CCACGGGGTTGGGTGACACAGCAGGTAACTGTGACACAAAGCTGCTCCTTGCTTGTTCAGATGACACCTGTTTTCTGGTTCAGAAGT
TTTGCTCATAGACCAACAAGATGGCTCAGCAAGTAAAGGCACTCAACCACCCATGGCCCGTACACACACACACACCACCACCACCAC
CACCACCACCACCACCAGCTGATAAGGCCCAAGGCAGGGTGGCCGCAGTAGCCTGGCTTTGCCTACCTGCCTGCCTGGCACCCCAGC
TGGCAGTGGGGTGTGGGCCAGGCTTCCACAGTCTGGAAGAGGGGGCTAATAAAGGATGCAGGGTACACACAGAGGCAGGCCTGGATT
GTGAGCAAGCTCAAGCTAAGTTCTCAGCAGAGTGCTTGCTTGACCTCTGGCAAGTGCATACAAATCAAGATCCTTGCTGGAATGGAA
AAAGGGGGAATTTAGAGAAGCTAGCAGGAGCAAATTTGGGGAGACAGGGTAGAAACAAGATTTGACTGTGATCTCTGAAAGTCTCCT
GAGGAACAACAGGAAGATCCTAGGCTAGCTAGGGCGCTGGAAGGAGGAAGAGCCAGGTGGCCCGGGCCTCTGCGAGGGTACCCTTAG
TCGGAACCAAGGCATAGAAGGCATAGAAGGAGCCTGGGACTGGAGGAGCAATGCCAGCCTGATCCTGGGCAGCACCCTGGCTCTTCT
CCAGTCTCCAAGGGAGAGGAGTAGTTGTACCTCCCTGATGGCCCCAGGCCTTGGGCACAGGCCAGCCCCCACACAGCTTCCAGGTTT
GTTCCTGCTGGCTGCTCCGGAGCTACCTGACTCCTGCCCAGCCTCGGCTCTTTGTTTGGCTCGGGAGGCCCCACCCCACCCCACCCC
AGCACACTCCTAGTCTTTCTGGTTCTTCAGGTTCCAGCCTCTGGTTTCACTGGAGACACGTTGAGGCAGGACCTGGAGGGGCACTTA
TTCCCTGGGTTTCTCTCTTGCCTAGAGGGAAGGAGTACGTGAGGCCCCAGCCTTCTACACTCCTCTCACTCCTTCCTCTGTCCTCTT
CTTCCTGTTCTGCACACCTGGGTGTCAGGAACTGGGTCAGACAGTGGGACAGGGCAGAAGACAGGACAGACATGGAATCTGCTCTGC
CCAGGGTCATGGTCTGATTGGGAAGCAACTATTAAACACCAGAAAGGTGGGAAGGCTTTAGCTTCCTGTGCACCCCGAAACTGTGGG
CAAACTGTGGGCGGCAGAGCGAGTTTGGTGGGGGTGGGGGTTAGGGAGGGATAGGGGAGAGTTTTCTGAGACGATGACGTTCAGAGG
TGACITAGGATGTTAGTGGGTAAGGGGTAGTCGAAGAGGCGCTTAGCAGAAGGTGCAGCTTACGCAAAGGCCCTGAGGCAGCATTGC
TTATTCAGGGCTCTAAAGCCAGGACTCTGGGGAGCATGGTTGGGTCAGACCTGGCAGAGCTTTGTCTGCAGTGAGAGGAGCTTACTC
TTTGTAAAGAGCAGTGGGAGGCCTTTGGAGAGATTTGATCAGGAGGAGGCTTGTTCTCTGGCTTCAGAATAGAGAATAGAGGAGGTG
GGGTGGGGGTGACAGAGGCAGGGCCATCAGCCTGTTCAGCCAGCCAAGCCGTTTGTCCCATGGACGGGATGCAGAGCTGGAAGCTAC
TGGGGTGGGGGAGGGGTTAAGAGATAACCTCAGTAGACCCTGGAGGAGGGGGCTTCCTCTCAGGCAGGTATTGGCAACAGTGATAGG
CTTTGTAAATTCTACAGACTAGGAAAGATCCACACCTCTCTCCAGAGGCGGGGGTCGGGGTGGGGGTGGGAGGGTGGTAGAAGCAGA
AAGGAGTTCAGAACTTTTGGGTCACCTGCATGACTTCCTGGCCCCTGCCTGCACTGGGCCCTCTGAGTTGCTCAGCCTCCCCCCCCT
AGGAACAGATTGTGGTGGGTCCCGAGCTTTCTTCCTGGGGAGGACACAGGTTATTTAAAGTGAGTTTAGGATTTAGAATGTAGAGTG
AACCCCAGCTCCACCCCAGAACACTGACTCATTGGTCCTTGTTCCTTCCACTGCTTGGATGTCTTTCCCTATCTCTAAAATGAGGGT
GATTATGGATCCACTGACCCACAGGGCTGCTGATGTTAACAAGTGACTGCCTGCCCTGGGTGAAGGCCTGCCACTGACACATGTCCT
TAAAAAAAACAAAAAAGACCAAAACAAAACCAAAAAAAAACCAGAATTGGTGCTGGAGAGCTGGCTCAGTGGTCAAGAGTGCTTGCA
GCAAGTGATCTTGTAGAGACCTGGGTTTGCTTCCCAGCACCCACATCAGGCAGTTCACAGCCACCTCCAGCTCTAGAAGATCTAATG
CCCCCTTCTGGCCACTGTGGGCATTGCACACATGTGGTGCACATACAGACAAGCAGGCACGTATACATAATACAGATAAAACTCCAA
GAGTTTCTAAACCTTCCTCCAGTTGGCATCTCTTTCCTAGTCACAGCGTGCTGTGACAGTCACCTGTGCCTCTCCCTCACCTTGGCT
ACTCCTCTGCTTTTGTCTGACTTTTGCCACCACTGGCTGTTCTCTTCAACAAGGTCCCCTGCCTCCTGTGAACTTTTTGTGTCAGGT
GTTTTGGACTTTGATGGGCCTACATTTGGGTCCCAAATGTGTCAGTACTCACTGGCTGTGTGACTCTGGGCATGTGACTTGGTCCTT
CAGCCTAGAAAGTGGAGGCACCGGTTGTGCATGAAGTGACATAAAGTACCTGGCGCACAGTGGGCACTTTTCTGCCAGGGCCCAAAC
TGAGCCTGGGTTATGGGCAACAAGCAGGTCCTCTGAAGGGCCAGCTCCCAAGGCACTGGCTTTCCTGCACCGTGCACTGGCTGCACT
AATAGGAAACGGATCCACAGTCAGGATTAACGAGGCATTGCTACGTCTCAATGAGCCAGCTTGCTTGGGGCTGTGACCCTGGGTGGT
GGGACATGGAGGGTATGGCCAGGCCTTGGCTGAGCCCCTGTCCTCCTCCTTTGGAGTAGGAACTCTGGAGCTGCCCATGCTGTGGAA
AGGCAGTTGCCAAGCCGGTGGGGGCAGGGAGTTACAGGAAAGCATCCCTCTGGGAAGTCATCCAGAGCCTCTCAGGAGCTGGAATGT
CCAGGCCTTTGGGGCTGTTGGAATGCAGGGGTAGGGAAGAGGAGGAGTCCCGGGGTCTGCCTAAGGCTTAGCGTAGATGGAAAGGCT
CTAGGAGAGTCTGGGGCACTTCCCAGAAAGCAGTTCTCCAGTATTGTTTTTATTTTTTTGTTATTCCCGGAGCTTGGAATAACCAGG
AGCCGGAGACAGACGGAGGTCGAGCCCAACTCCAGACTCTGCTCTTGACCAGCTGTGTCCCAATGAACAAATCTCTTGACCTCCGCA
CACCTTGATTTCTTCTCTGTAAACAGTAATTATAGCACCCACCTCCTTGGCTTCAAAGAGTTACAAGGGTTCCGCTCTTTGAACAGG
GCTCGGCACTTATTAAGCACTATATAAAAGTTAGGTATTATTGTTGTTCTCTCTAAGTCTTTCTTGCCCCCTCGTCCCCAAGGCCTG
CTACCTGTAGCATGGTAGCATTGACCATGCCTGGTGTGATCATGCCACTTGATTTGAGTGTCTCTGTCTCTTCCAGGCAAACCCGTC
TTTGTTAAGGTCCCCGAGGACCAGACTGGGCTGTCGGGAGGGGTGGCCTCCTTCGTGTGCCAAGCCACAGGGGAACCCAAGCCTCGA
ATCACGTGGATGAAGAAGGGGAAGAAAGTCAGCTCCCAGCGCTTTGAGGTTCGTCCTGGACTGGCCGGGAGGGCTGGGCAGGCTTTA
GAGTTGGCCTGCTCTCTGTGTGGTCCTTCTCTTGTTCCTCAAGGGTCTTTTGGGGATGTGAGGAAAACTATAGTAGATCCACACACC
CCTAACAGGCTGTGACCACGTCTGTCCTGTGACAGGTAATTGAGTTTGACGATGGAGCAGGGTCAGTGCTGCGGATCCAGCCATTAC
GAAGTGCAGCGAGACGAGCCATCTATGAGTGCACAGCCACGAACAGTCTCGGGGAGATCAACACAAGTGCCAAGCTGTCAGTGCTTG
AAGGTATGTACCAAGAGGGACAGGCCACCGTGGCCAGGTAGACTGAGATGTGGGTGGGGCCCACCAGGACTGGCTGCCCTGGGGTGG
GTGAGAGTCAGTGCTCAGCAGGCCAAGGTCACTGGTCTGTGTTGAAGCCAGCAGCGAGCTTGCCAGCTGTGTGATCTCCTTCCTGCT
GGGTCATCGCTCCCTTGGGTCTGGGTCAGGCTGGAGGTATGGTGCTGCAGCTCTGTGGCCCTGGGGTTACGTGACGGTGGCAGTGAA
GCCTGGCAGTTGCTGGCAGTGTTCTCTTGGTACCCTGATGAAGGAGCTGGAGCTGGCCTCTCCATGTGAATTTTAGTGTCTCTTCTC
ATCTGGTGGCTCGTTCATTCATTTAGTCATTCACAAACTTTGCTTGAACTGCCGTTAAGCCCAGAATGTCACTTGATACGTGCTACC
CTCAGTCAGTGTCCAGTAAATTCTGTAAGGAACGTGGACACTTTGCTTCTAGGCCTTTGCCAAGACCCGGGGTGTAAAGGGGAGCAG
AGCTTCCAGAACTCTGAGTGGTCAATTTGAGGCTTTTCAAGTGGCGGCTGGAGAGCGGAGGGGAGGGAGAGGGGGGAGGGTAATTCA
GTGAGGCTGCAGGATCAGGCTGAGGAACGGGCCCTTTCTTGAGGACGTAGAAAGCCACAAATAGGGTGGGAAGTGACAGGATTAAAC
CTTGACCACAATGGATACATGAATCTGAATGTGCATTTTCAAAATACCGTATTGATTGTTGCTTTCATTTTTGAATGCAATACCAAT
ATCTGTTCACTGTTAAAGAATTGAGAAGCATAGGATAGTGTGTAGGGAGAAATAATAATCTCTTATTATCCTACCCCTAGTGCTATT
AGTTATTTCTTGGTGGTCCATTTTCTATGTGTATGTTTTATATACATCTATGTATATTTGTATTAATTTATATAAACAGAATAGTGA
TCACACACTGTTCCATGACCTTGGGGGTTTTTTTTTTGTTTGTTTCTTTTCCAGCTAGAAGGCAAACCTAGGGCTTTTCAGACAAAC
TCCCTCACTGATTCCTGGAGCTCACCTATTTGGCTAGCCTAGTGCCAGCGAGCTCTAAGAATCCACCTATCTCTGCCTGCCTCCCCA
GCACTAGAGTCCAGGCACTCACTTCTGTACCCAGCTTCCTCCATGGGCTCTAGGAAACACAACTCAGGTCTTCACGCTTGCTCAGCA
GGTCCGTTACTGAGCCACCTCCCCAGCTCCTCCTGCTTAGGAAAAAACATAGTGTGAAGATTTTCCTTGCCATTAAATATTCTTCCA
CACCATTATTTTTAATGGCTGTGGTAAGCTGTCACATGGAAGCACCCTAATTTATTTAGCTGTTCCCTTTTTGTGGGGTGCATATGT
TCTCAGCTTCCAGCCTTTGTAAGTACGGTGTCACACAAGTCTGTGACGATGCCGTTGCTTTGCTGGTCATTTCCTGACTCTAGGTTC
GTGGAGTAGAATTGCTAAGTCAAAGGGCACATGCATTTTAAAGCCTTCAATATTGCTAGAAGATTGTATCTGTTTATATTCTTATCA
GGGAGGGCATACGAGGGCCTCTTCTCTGCACATCTTCACCAGCAGTAGAAATTACCATTTTTTTTTTTTTTTCTTTGCCAAGTTAAT
AGGCAAAAAATGGCATCTCAATTTAATTTGCATTTCTTTGATTACAAGAGCAGTGGAACATCTTTTCACATTGGCCATTTTTATGCT
TTGGCTTTATGTGTTCACACATGCATCCATTCAGTAATTCTTGCATCTGTGTGTCTCGTGTGAATCAGTGGGTTCTAATAGAGCTGT
GTGCGGCCCTCCCCTGTAGACAGGAACGCTGGGCCTCATTTGTTCCATCCGGGATCCAGGACATCGTACCCACAGTCCAAGCTAGTG
TGGAGCTTTCCCTGTAGCCCAGGATCTCAGAGTCCATTCTGAAGTGTAGACAACCTGAGCCGCTGCTGCTTGGTGTCACTGGGTTGT
GCTAGCCCTGCTGTCCGTTCCATTTGGGGACAGGGGGCCAGACAACCAGGTCTGAGCTGAAACCACTGGCTTGGAGGTTCCTTCTGG
TCATTTCTGACCTCCAATTTGTGACCCAGGAGAAAGGATTTCACCTGTTGGTGTGAGAGTCATCTACCCTGCCTTGTATTGGAGTGG
AGTCTAAAAGACTAGAACTATAATCCCTATCATGGCGGGCAACCTCTGAGGCTCTCCGGCTAGGGAGTAGTCTCCTTGTGAGACTGG
AGAGCTGATTCCTCTAAGACATCCCACTGCCCTGTCAACTTTACCCTTTAAAGGGTCTCTCAAGGCCCGGGGAAGAGAACTCACACT
TGGCATAGGGTTGCCATTAGAAACGGCCTTCCTGGTCAAGAGTTTCTTTCCATGGATTAGAGGGTGTTCTTTCTTATGTGGTGTTGG
GATGACCCCGGTACTAGACATGGACTTTTATCCCCAGACCTGGGCTTCAGGCTATATGTTGATGCATTCTGGGAACTGGAAGATGTC
TGATGATGTGTGAGACAGACAGGTCCCTACTACACCAGAGTGGTTGGAATTGTGGGCTTCGTGGGCCCCTCCTGCTCCTGAGATATT
GCACCAGCGAGGCAAAAGTGCTGACCTGTCCTGTTGTCTTTGTAACCTTGACCAGCTTAAGGCTTGGCAGAAAGGAATGTCACAGGG
GATAAGACCACGGAGAGGCAGCCTAAAGCAACCCTGCCTCATTTCCTCCCTGTTCTTCTGGGGTGGGGGGCAGGAAAGCTGGGGTGC
TTGCCTGCAATAGATGAAAAGATGGCTTTTTTTTTTTTTTTTTTTTTTTTGGAGCTCTGCGGTTGACACGAGGGGAGAGCCAGGGCC
ATTTTCTATGGAGAGACTACCATAGAATCCTAGTAGTGAGAGATAAACTTGGCTTAGGGGCTGGAAGATAGTGCCTGGTTGCTTTGC
TCTCACGTCTCACACACATGTGCCCGCCTGTGCATCAGGAGACAGGTGCTTTGGGGAGTTCCTGGGTAATGCCTGGGGAATCTGGCC
CAGGTGCAGGAGCAGTGTGTCTTCAAAAGGGAACCCCAGTATTATACTTCCTTTGGGGCCTTCTGCCCTTCCTCCTACCACCCCAGG
CCCAGCTCAGTCCACTCTCACTGCAGCCTCTGGGGACACCACCATTGCCAGGTCCCCCACGAGTCTCTGTACTCTGCCGCTGCTGGT
GCAGCTTGTGAGACCTAGCCCACTATGCTGGGATGGCCAGCCGGTGTGCCAACATCCCTGTCAGGGTGCAAGGTGGGGTGTCCAGCA
CAAGCTGGCTGGGGCTGGGCCTGTCCTGTCCTGGGCTGTGATGAGTACCAGGGCCCTGCCGCCCAGCGTTCCTGTCTGAGTGGTAAT
TGAAGCCATTAGCGCGCCAGCCTCTCCCTCGCCGGGTAATGGCAGGAAAAGCTCTTCTCGCTCCGCACTCTTGAGGCGGCGGCTGAA
TCATTCCCCCTCCAACCCGCCCGCTGCCGCCACTGAGACAGGGAATCTGACATTTTCCCTCACAAGGGAGGGGAGGGGTGGGGGAAG
GGAGTGAGGCCTTGATTCTGGGCTTTCCCTTCCGAGGTGGAGGGGGATAGTGAGGGTGTTGAGCTCAGTTTGGAGGTCATTAGAGTC
CCCAGGGTTTGCAGAGTCCAGGCCAGGCAGAACCATGGTTGGGGAGAACTCCAGGGAGGAGGCTAGACAGCCAAAGCTGTGGTGGAG
CCCTAGTGACTGGGTAAGTTCTGGAAGGGACACCGAATGCCCAAAGGAAGATGGGGACAAGAGGCAGTGGAGTAGGACTTGTCCTGG
GGGCCGGGCTCAAGGGCTGGATGTTTGTCATTTCGTGCACATAGAAGATGATGTGCTTACAGTGGTGTGAGCATGTGTGAAGGGATG
TGTATACCAGCAGTGTGCCTCAGGCAGCTGGGGAGGGGCATGATGTGTAAGCAGGTGGTATGTACTTTAAGGGCTCTGTGCAGTAGT
GAGATGTGTGCATGTGCACGCATCTGTGCATGGCAGGTAGTATCTAGGGTGAGTGTGTGTGTGAGTGAGTGTGTGATACGGGTGTAC
AGCAGGTGGTATTATACCTTAGCTTTGTGAACAGCGAGAGAGTCAGCAGTCTGAGTTGGATGTGTAGGGTCAGTCTGGTGCTGGTGG
GACGTCAGGTCCCGTATTCAGATTTGTTGTTTCTGGGGTCCCACAGAACAGCGGACAACAACGAATGCTGGTCAGTGTGGTTGGGGC
TAGAGTGTTGTTGTAATCTGCTGCCATCATACTCAGCAGCTCCTCTGTGACCAAGCCAGGAGGAGGGCACACTAGGAGTCCTGACCA
AACCTGGCCTTAGGATTTAGGACTTCTCCTGTCTTCTCCACCCCCCACCTACACTTGTCCTTCTACCTGCTTCCATCTTGGGCTCTG
TGTCTGCTAAGTGGGCCAGACTGTACCACCTATGCCACTTGGCTGGATCCAGCTAGAGTGTCTCACATTGCCTAACCTGGAGAGCAG
GCAAGAGAGGGCGTTTCAGTTAACACTGGATCGTGGGTTCTGCATTTGCTCTCACCCTCGTGGGTTCTGAGGCTTTAGGGTGACAGA
TAACTTCTTGCCACACACGCTCGCTCAAGTGTGCGCGCACACTTACACACACAAACACACACCTCCCCCAACACACACATATGATCT
TTGGAAGCAGCTCCAGAGGAAAAGACTGATGGTGACCCTGTCCTGAGCTGTCCTGCCTGAGCTGGGCAGGATGAGTGCTTTCCTAGG
GGTCATTTTTCCCTGCCCCACCTTTTTTCCTACCATAAGCCTTTCTATTCCTTTCACTCTCAGTGGAAAGATGAGGGTGAAAGAGCT
ACTACAGGAAGTCCCAAACCTAGAATTCTAGGCAACCCCTGTTCTCCCCTGGCAGCAGTGACACTGATGTACCTCTTGTCCAGTGGC
CTTTAGGACGTAACCTCCATTTCCTGTGACCACAACAGATCTCAGGACCATGAAGATATGAAATACTATAGCCAGATTTTGGAGGTT
CAGACAGAGATAGTCTTGGGTAGGAAGGCACATCTGGAATGGGGCAGTAGAGGGTAGGAAGGCACATCTGGAATGGGCCAGTAGAGG
CAGCTGGAAGGTCAGACACGGGGTTTCTTGGTTCCTGGAGAGGATTCTCATGGTTTCCAGTTCTGAGAGTCTTCGAAGAGCTCAGTG
AACTCCTTTTGAACGGAGTCCCCTTCTGTGAGCTGTGGCCTGTGCCCACCTAGTTCCCCACGCCCGTGCTCACAGGTGTCTCGTCTG
TGCTCTGTGCTTTTTGACACCCCCCCCCCGTCTCCCTGCCCAGCTTATGATCATGGGCTGAACCAGCAGCGCTAGAAATAGCTCTCA
AAGTTGGAGGTCTTAGATACCCCTGGATGTATATTCTCCATCTGTGGTGAGGTATCCTCCTCACCCTGATTGCCAGGGGAGAAGAGG
GCTTACTAACGGCAGATTCATGCTTCAGAGAAGTCCTTGTTGATGCAAAGTAGGGCGGAAGTGTATCTATGAGAGGTAGGTGTAGTC
AATCCAACTAGCAAAAGCCAGCATCACCCGAACCTTGTGAAAAGAAGAGGAAGGGAGATAGATCAAGAGCATGACAGGTAGCATCCA
GCCCAGCTGCCTGGGACACCCCAGGGGTGGTTGGCTCTGCATTCCTGGTTCAAGGCTGGTAGACAGCGGAACCAATTTTCTATCTAA
GATTGAAATACAGGAAGAGGCCCAGGACTGAAGAGGTGAGTTGATAAGTCTGATTTGAAGTCTGTCAAGTCAGGTGTGTGCAGGCAT
CCAAGGAAAGATGTGAGAAGGCCECACATGGGTTTGCCTGGCACTCTTGAGGGGCTGGAGATGGAATCGTGAGAGCCAAGAAGCCAA
GCCAAAAGGATCCCCAGGAACTGGCGTTCACTGTTGGGAGGAGGTTCCTGGGAGCTAAGGGGGAGCCAGAGCAGGGGAGGCAGTGGA
AGAGTATGAAGGAGGAGAAGCCAAGCAAGATGAGTATTGGGCAGTGTCCTTTGGATTTAGCTCTGCAGTTGGCACTGACCTTGGCGA
ACAGGCTTCCAGAAGCCTCGGCGGTGGCAGCAGGGCTGCAGGGGGCTGAAGAGCGAGTGGGAGGCTGTCAGTGGTCTGAAGTGACTG
CGAGCAAGGGCTGAACCTAGAGAGGTAAATGGCTAAAAAGAAAATTCCTGAGCAGGCTTGGGGTACAGGGGCATCAGGAGGGAGAGC
CCAGCATGAGCAGAATCTGAGTGTGGGGACCAGGCAGTGGCTCTCGGGTCCACTGAGTCCAGTCCGCACGGTGGGGAGGCCTCACCT
GCCAGGTTGAGTAGGACTCCGTGGAGTGCCAGGGAAACCCAAGGGAGGGGGGATTACTGAGTGGGGGAGGCCGTGGCTGCCGACAGG
CCCCAAACAGCTCAGACTCAAAAACAAAACAGGCTTTTAAGATGCCAAGTTTGATGAAATCTGAGTGCTGGAAGAGGTGGGAGTTTT
CTCTTGGAAAAACAGCTGAAAGTCGAGACTGAAAGCTGCGAAGGGAGATGGGCGCTGAGGCCCCTTGGGCCCCCCTTACCGCCTGGG
AGCCTGGGGGTGGGGTGCAGGCTAGGCACTGAGCGGGAGAGCTTACAGGTTTGGGGAGGGCTCTGTCTTGACCCTGTCTCTGCTGAG
GATGGAGCTGTTCTGGGCGGCTGAACTGAGGGTGGCCCTGGGCTTCCTTTGGAGTGCCAAAGGGCAGGGTGGGGCCTGGCCACCCAC
AGAGGTTCCTTTGTGGGTTTGGTCTTACTGTGTCTCCAGGTGCGTGTGTCTCTTCATGTGTGTGTCTGACTTTGCATGTCCTCGCAC
CCGTGTTCCCACACCTCTGCATGCCGCCTCTGGATCTGCCTGCAGGAGTCTGTTGCCACAGGTGCTTGTCACTGCAGATGCCCCGCC
ACAGGCACATGGAGGTTTCTATTTTTGCACCTGTCTCTTTCTCCTCGTAGTTCTCTAGGTCCCTGGCTGTGCGTGTTTCTAAGGTGT
CTGTGTGTTCAGGGACAGGGTGATACACTCATGCATAGAGGAGGGGTCAGCCTCACAGACTGTGCCACCCCGGGGAGCCAGTCTATG
TGCATGCGCATATGGGCCTGTATTCAGAGATGCCATGAAGCTGGGTAAACACGGGTGAAGAGGTCTCTCTGACAGACGGTGCCAGTG
CTCTCCCTTACAGCCACCACCCTCCCCTCTAAGGCATCTCCATCTGTTAGCTTTCCTGGTGCAGCCCCCTGCCTCTGGCCCAGACTC
CGCCCCAGGCATCCCTGGCTTCATGGACAAAGAGACCTGTGTGTGTTCTCTAGAGGCCTTCAGCAGCCTCCCTCCCCCAGTCCTTGC
AGCCTGTCGGTCTAAACTTCCCACCCACCCCTGCCCTCTCCTTGTCTATCAAAACTCCAATACATACCCCAGACTCACCTTGCTGTT
TAGTATTCCTCCATCCTGGCTATAGTCTTTACCCGGCAGTGTGCAGGAAAGCTTTTTGCTTTGCATCATTCTTGATCCTCCTTCCTC
CAGGAAGTCACCAAGATAGCAGATGAGAGGTGGAGTTATAGTAATTAACTCTACTGCTGAGGGACATCAACCATGCTCCCTGTCTCT
GTCGTTTGTATCCTCCCCACCCCACCCCTTAGACACGCCCTCCCACATGCCCTGTCATCTCTGAGCTGTAAGACAACAGGACCTCTC
CAGGGAAGGGTCTCTCAACCGCAGTATCTGAAGGGCCAGTTGGGTACAGGAGCCATTCATGAACCACTTGGATGCTTACTCTAGGTG
CACAGATCTGTGCCGGAGGGGCAGACTTTCGATTATGTGTGGTACTTATGTATAGAGGCTATCCAGGTCTAGGCTACTGTGTTGGTC
CCTCTCCAGAGTCCCCCAAAGCTAAGGGACTTCTCTTTCTGCTCTGGGAACAGTGCAGTGGAGGACCTGGCCGACACTGCGGACTGG
ATATGCAGAGGCAACTTAAGAGTCTTCTGCACTGTTCTGGACCCTAGCAGGCGGGAGCTGGGTGCGCTGCGTTGGTGGAGCTGGAAT
GCCTAAGGGGGAGCACCCCCCCAACTGAGGCGCAGGGTCCAGGGAGGGGGCACGGCTGCTCCGCCTAGGTGGGGGCGGGAAGGGCGG
CGCCCAGTAGGACTTGGACGCGGAGGCGCGCACCCGCTCGGACACAAGCGCGCGCAGCCCTTCCACCGTGGCACCCGCCTCTTTGCC
GTCGCCATGGTTACCGGGTGGCCTGGAGGGGGGAGGGGCCCTCCCGCACCAGCAGCCCGTGTTTGGAGGGGCTATAGCGTAACCCGT
GCACCCACACACACACAGGCGCGTGCCAGGGATGTCCACATGTGGGGAGCATTGGGCTTCCTTTACAGCACCTGAAAAAACGTAGAG
GCGTGTGTGCATGGGGGGTCTGCAGTAGAGTCCGTAGCCGGAAGCCGCTTAAGGCCGGGGAGAGGGGGAAGGGCGTGGCTCTTCCCA
GGGACGCCCCTCCCTTCCCCTGCCCTGCCCACCTTTTCGTGAATGCTAGTCTAAAGCAGCAGGGTCCTACCACCTGCTTTCCTCCAA
GCCACACCTCCAATTTAACTGAGTGACCTTGGGCAGGTTTCCAGATTTCTGCCAAGTCCCCATTTGCTCATATGCCTCATAGGGTTT
TGGTAAAGATTGTCATTTTGTAAACCTGAAACAGAGGGACTGCTTCATGCTTGGAACCTTTTATCCCTGTGTCCAGAGGCCTCAGTT
TTCTCAGTTGTTGATGGGTTGGAAGTGACAGTGTGGCATGCATCCTCTTGAGAAATTTGCCTTGGTCATTAACCTGAGTGGTTTGGA
CTTCTATAGACACAGCTCTGGTGGTGGTGGTGGGGGGGGGAGGCTTAAGGTGGCAAGGGGGCCAGTCACTCCGCAGGCATCTGGGGA
ATGTCTGTTGTCCTTGACTGATAAGAGTGTGCTGAAGGCTCCCAGGTGGGAGAGGAGGAGGCTGCTGCAGCAGCAGGTCAGCCCTCA
GGGGCTGACTTCCCCTCCTGGAGTGGTCTAAGAAGCAACCTTGCCTGTGCTCACTGCTTGGGAACCTAGCTGGCCCCAGGGACTTGC
TTTTGGGTCCTGCTGTTCAGTAGTCGCTCTTCACACTCCCTGGTTTTAGGCTTTGGCAGTGGCGCCTAAGGAATGATCTGAGCCAAG
GACGGAGCCATTTGAGGGTGTGATAATTGAGGGAGGAAAATTAATTGTCCTTAATTTGGCGTAGGTCCCAAAGACCTTCCCCGTGTC
AAGAATTCAGAGTAGATCCCTGGGACCTACTCTGCTTTCATTCTAGATCAGTGTTTTGCACGTAGGCCATAGAGGCTGGAGAGCTCC
TTGGCTCTGACTCTGCCAGGACCTAGCCGCCCCTCATCCTGCACCCCAGTTGTTCAGACAGACAGGAGTAAGCCTGGAAGAACACGC
TCGATGCGGTGGTCAGTGATGATAGCTGTAGCTCTAGCAGCCTTGGGATCCCTGATGCTAGATGGGGATGGGGAGGTTGCCCTAGGC
CTCAGGGCTTACTCTTAGCTTCCAAGTGGTGCCAGGCCAAGGAAGCTGTGCATCTTCAGTCCTCACTCCCCTCCTCTTTCTCTTCCT
GCTGCTGTTTAATATGATCCTTTCAAGAATCAGCTGAGCCGGGCAGTGGTGGTGCACGCCTTTAATCCTAGCACTTGGGAGGCAGAG
GCAGGCAGAGTTTGAGGCCAGCCTGGTCTACAGAGTGAATTCCAGGACAGCCAGGGCTACACAGAGAAACCCTGTCTTGAACCCCCC
CCCCCCCCAAAATCAGCTAAGAGTCAGAGCTCTGTGGCATCATACTTACAGCCAAAGAGACCCAGGGTTTACATCAGAATTTATAGG
GCTCCAAACAGGGAAGTGTATAGCCAATTCCTGGCCAAACCTGGGGATTCAGGGCCATGATGAGAGATCTCCTATAACCCCAGCCAG
ACACCCTTCTAGGATATCCCTAGAAGTTGCAACTTTGATGGAGGTTCCTCTGCCTGCGGTGGCTCCTCCAGCTTCATCTTGTCTGTG
CCCCGGGTGCTGTCACCCTGCCCTTTACTTTTCTCCCTCTGAGGCTGAGTCTTCCTGGCATGGGAGCCTGGCTGCAGCAAATGTTCC
TGCACACTGACCTGTGCTGGGCATGTGAGAGGGAAGAGTGGAGTGGTGCCTCTGCCACCACCATACTGCCCAGCTTTCTTGCCGAAA
GGTAAACAAAGGGCAGACATTCTCTAGTCCCTCATTAGGTACACCCAGAGCCAAGCTTGGGAAGAGGAGAGGTACTGTTGAATTCTG
GAAAACACTTGATGCTTTTGTTCCAATGGGAAACCATAATGGGAAAGGGGGCCCCAGGCTATAGTTCTGGTATCTGTCTGCCCCAGT
GGCTCAGGACTGGTTAATTATAACTTCTAACAGTTACTGGGTGCCTGTTCCGTGTCAGATACTCCCTTAGCACCCTGGATTCTTTTT
TTGGTGGAGTCTTCAAGGTATCTTTACGAGGTGGACACCTAAACCCAGTAGATAAGTCAAGCATCAGTGAGTAACGAACACGCATAG
GCCGTAGAAGTGGTAGTCAGTGGTAGAGACGTCCGTCCCACATGGTCTACACTGTCCCAGGGCTTGTCTTCCCAGCTCCTGGTGAGA
GGTGGGCATCGGGCAGCTTTGTCTCAGCTTGTGGGGACTGCTTCAGGAACCTACGGCAGGAGGAAATGGGTCAAGTGAAAGTGATTT
CCTTTGTGAGTCGCTAACTCCAGGAAATAGTGGGAGGGTTGGTTTTGTTTTTGTTGTTGTTTTTACTGTTTGGTTTTTTTTTTTTTT
TTTTTTTGTCTTCTTAGCCTACACATACTCAGACATGGCCCTGACACCTTATCCTGGTTCTTGGGGCCAGGAATGAGTAAGAAAGAC
CAAGGGTCAAGACTATAGTAGAGGATGGTCAGAGGCAAGACTAGAGTTTGGTCATCGGTAGGGCAGGGCCAGGGTCAAAGACCTGAG
GCTCAGCCACATGGAACAGCTGAAGAGATGCAGGCCAAGGAGAGATAGCATGTGTGCTGACAAGGAGCTGCGGGCTGGACCAGCACA
CCTGGTGGGGTTGTGGCTATTGATGCTTGGGGGACTTTACTTCCCTTGCCGCCCCCACCCCGAATCTATATAGCACTTTGCCACAGC
CAGCAGAGCATCGATCCCTGCTCTTCCCAGGACCTAGAGAGATAAGTGCTGACGTCTTTAGTCTGTGCTTCCAGACTTGGCAACTCC
CCAGCAGGGATGTGGTGCTATGGGCCCTCCTGCAGCCCCTGCCTTGCCCAAAGGGACCTAGGACTCTTGACTCCTCATTTGGGGTAG
GGCTTGTGTTTAGAAGTCAGGGGTGGGAAAGAGAAGTGTATCATGGGTTCCCTGCTAACCTGGAAGTCAGGAGAAAGCTACACAGAG
AGGCCCTCAGTGGTGCAGAGGCAGTCCTAATGCACGCGTCACTCTTTGTTTGCAGAGGACCAGCTGCCGTCTGGGTTCCCGACTATC
GACATGGGACCTCAGCTGAAGGTGGTGGAGAAGGGTCGCACTGCCACCATGCTGTGTGCAGCCGGTGGGAACCCAGACCCTGAGATC
TCTTGGTTCAAAGACTTCCTTCCTGTGGACCCTGCTGCAAGCAACGGTCGTATCAAACAGCTGCGATCAGGTGAGCTGAGTTAAGGC
AGAGTCCTGAGTACCTTGGGGAAGGCACCTTCCAGCCTGGTCCAGCCAGGTGCTGGAGGATGAGCTTTCTAAGAAAGACTCATTAGA
ATGTCCCTATTGTCATAGGGGACAGGATGTGGTGGCTAGCGCTGTCCTGATCCTTCTGGCATTGTCAGGCACCACACTTTAGGCCAG
TTTTGCTCCTCCAGGCATGCGTGACACGCCAGTGTGGCAGGCCTGAGGAAGGTGCCCTGGCTTTTCATCTACTTTCCTAGAGCCCAG
TCCACGTCTCTGCTTCTCTAGGCAGCATGGCTTCCTGGGGAAGGGGCCAGTGATACTTAATTCATCTGCCTAAGAGCCTGAAACTAG
GGGCATCTGCCTCTGTGCTGTGGCCACCAGAGCCACCTGTTTCCCTCCCGTGGATGTCTCAGTCGAGCCACCTTGGAGCCCTGGGAA
CAAGGATGCCATTGTGACCAGGAGGGAAGTGTCGCCCCGTTGATCGATCTGCCTGTGAGGCTCCCGCCAAGAAAATGGATTTTGATT
TTGTAGGGATGGAGTGGGTGGGAAACGAGGCTCAGAATCCGCTGGGCTGTGTTCTACACCCGCTAGCAGCTCAGGACAGACCCACAG
ACTTGGCTCCAAAAGTCCTGCTGTCTGGGAGAAGGTAGGCCTTTCTCACGTGGCTTGACTTCCTGCTGCCTATCAGCTGTGATGTCA
ACACCATGAAGGATGGTGTGTCTGCTTCTCTTAGGGCTCTTCTGGCTTCAGAGGTACACACCAGTCCCAGCCCCTCCTTAGCCCCTC
CTTTCAGGCCCACACCATTTTGGTACCCAAGAAGAGGAGGGAGTGATGAGACACCCAGCAGGGCAGGATTCCGGCCCCAGCCACGGC
CGCCTGAGACAGCCCACGAAGTGTTAGCTCATTTAATTTAATTAAAACTCAACAAGATGGAGGCAGCTGTAGTGCAGTTAATTAAAA
CAGCCATAATCAAGGCAGGAAATGGTCCGGCAGTGTTTGTGGCTGCTGCCCAGCACAGCACAGAGGCCGGCATGGCTCCGCTCCCCT
GTATTTGTTCATCGGTGCCCCCAGGCAGGTTCCCCATGCTGCCAGACGGTCCTCCCCGCAGGCCTGACTCCTCCCCGGAATGGCATT
GACTGTCTGTGGCAGGCAGCAGTTCTGGGAAGGCCCGCTGTCTGCAGCATTTGTTCATCAAGTATTTATGTAGCGCTTGCTGTGTGC
CAGACCCCGTGCCACCCCTCCCCTGCCCATGACTCCCCGGGATGCTGCGCAGGCCTCTTCCTTCTTGTCCGCACAGTGACCGACAGC
ACTTCCTCCTCCTTGAGCAAACCTCCTGCCCTCCCTCGGGCTTTGCCACCCCTGATTTCCTCGAGCTGTGCTGTTGTCTCCTTGCCT
GAAGCCCAGGTGGGTTCTTCTAGCCCAGAGACCTGCCAGCCTCACCTTTGCTCTAACAGTCTGTCCTCCAGCAGGCAAACCCATTGC
TTCTGGTGCTTCAGTCACCGCCTCCTTATCTGTCCTGATACTGGTCAACCCAGCCCAGATATCCTGTGAGCTCCAGGTCCCACATTT
GATGGGTGCTGTCTGGATTTCACCCTATCATGGATGAGCAATGACTTCTTTACAAACAGCCCTTCCCTGCTCCTGTTGACCATTTGT
CCATGAGTAGCAACCTCACCCCCACCCCCAGAGCCCAGCCCCCCATTTAGTAAGGATCCACATCCTCTTCATGCCTCACCAAATGGA
TCTCAGATTGGTGCCTTCTTTCTTTCTGTTGCCTCTGTCCTAGCCTGGGACACCACTCCTGTTCTCCCTGCCTCTAAACTTGCTCTG
AAAACTCTGTCCTCCGGCAGCTGTTGGTGTTGTCCATTCATCTTCCTCCTAGACTCCGAGAGCCAGCGCTGGACCCTGGGCTCCTAA
CCCTTGCCCCTGCCTGCTTGGTCAGATATGTGCTCCTGGTCAGGGACCCTGCTCAATCACATGCTGGCTTTATCTCGTCCTGTCCTA
TCTACATGCACCTTATTAGTTCTGTGGCCTTAGGTAACTTTGTCAGTTTTCATATCTTGCAAAGTAGCTGCATTAAGGACACAGTGA
CACTTTGCGGCCTGGTTTGTGGTAAAGGCTCTGTCCCTGCCTGCTGCCACTTACAGGTAACCACGATGACCCATCCTTGCCCACCTG
GCCCTCCGCTCACTTTAGCTTCTTCCACTGGGCACCTGCACCCAGCTTCTGACTACTCCCTCAGGAGCCACCCTGCTTCATGCTGTC
AGCGTTTGTCTCCAGCCCTACTTACCTAGGCTGGATGTGGGAGGAGCTGGGACAGATTTGCTTGGTAAGGTGCCCCTCCCCACCATA
CCCGGCTCTCTGTAAGTCCTCATTGAGGAATTACTTTATTGACCTTTGGGACTTATGACTGAAGATGATATCTACTCATTTGACCTT
TCCTGCTCCAGGAAGCTGTGACTTAAGGATCATAAATAGGCCAGGGAGGGTGTGTGCCCAGTGCTTGGGATGAAGCCGTGTGAGGAC
ATCCCTTCACAGAGTGGGCTCAGGCCCCTATTATACACTCGCCTGTGTCCAAGACCCCTTGTGGTTTTCATGTCACCCACAGTTCAC
AGGGTGCTTTTCTACCCTTATTACCTCAGAGTCTCTTGCAGCGTCTCTGCAGCAGGACAGGCCGTGTTGATTATGTCTACTTTCCGC
AGGTCAAGGGGTCACACGTGGAGGTCCTGGGAGGTAACCCAGGGAGGTCCTTCTAACAGCATATCCTAACCCTGGAGTCTGAGAAGT
AGTTGTATCCATCAGAGCTCCAGACATTAGCTCTCCAGACCTCAGGTCCCCCTGGCGGTCTCTACAGTACCGCCAGCATCTGTGAGC
CATAGCACATCTGTACACATCAGCTCTGTTTGAAGGGCTCTTTTCCCGCTGCAGCCATGGGATCCCTGCCTCGGTACCCCATGGGCC
ACTTGAGCTTGAGACCTCAGGCAAGCCCATGCAGAAAGGACGCCTGCTGTGGCAGTGGCCGAATTGGGGCTGTCAAGTGGAGATGGC
AGCTCTGCCTGCAGGAGGAGGCCTCTGTTCCCTCTGATGAGATGCTCCTGGCTTCTCCCTCTGCTGGCTGGCCCAGCTCTGACTGGA
AGAGCCTATCTACCCTGCCTTGCTCTCTCTTTGACTGGGTCTTCCAGAATGGTGCAGTCAGCATGTGAGTTCACTGGCACGAATCTC
TTGGGGAGGCTGACACTTGGGCCCCTTCAGTGCCATATACGTGTCTAGTGAAACAGAAAAGCCAGATTCTCAGTCAGTGGGTTAAGT
GGAGAGAGAACAGAAGCCACAGGCTTGGCTTCAGTGCCAGACTGTGCACACTACAGCCCCCGCCCCAGCTCCAGCCTAGACTGTAGG
AGGTCAGTGCTTGTCAACTTTCTGGTCTTAGTATTGCTAACTTTAGCTCAGAGGACCGTAAGGTAGTTTTGTTTAGCTGGCTTATAC
CTATTGATCTTTACCGTATTAGACATTTAAGATAGAAGATTTTAAATGTTTACTAATTATTTTAAAATAATTTAAAAACTATCAAGT
GTTACCATAAATAAACATTCTAAAATAAAAAAGATAATTGTGTACTCTAAGACAAAAAATATTTACTTAGGAAAACCTCACTGTTAA
TTTGGAGCTTCAGTGTGGCTGCGTTCAGCCTGTTAACAGCGTTACAGGTTGTGAACCCTTCAGGAAATGACACTGTATGTACATAAG
AGAAAGCATCTAAATAAGGCAGATAGTTCTGGTTTTGAAACAAGACCTTTACTGTATAGCCCTGGGCATGATACTCTATAGAGCCCA
GCCTCTCCTAGAACTTGCTGCAGTCACCCTGTCTCAGCCTCCCAAGTGTTAGGATTACAGGCAGACTCCACACCCCCTCCCCAGTCT
TAGTATTGTAGGAACTACTAATTGTAGGCTTTATTCTGGTGGACTCCCTAAAAGGTTCTCAGTGATTCCCTCTCCCCCCAAACACAC
ACACGCGCACGCATGCATGCACGCATGCACGCATACACACCCCTCTGGAGCACACATGGAGAATCTCTATGTTGGTCTAGTATCACT
TTCCTATATGAACACATCACTCCCTCCCCAGCCCCCACCGGTGCCTTCTAGCTTCCGTATCTGTAGGATGTCCGTCAGGAAGGCTCC
AAGTTAGACTGGTTCGATCCTGTTGCCTCACATAAAAGCCTTTTACTGTTTCCAGGCCTCCTTTTCCTCATTCGTGCATAGAGATGG
GTATTTTTTCCAGATGGGTGTTGCCAAGTTTGATGGGACAGGAACTGATCTCTGGCCCCAGCACTTCCAACCCAACCCCCACTGAAG
GATCGAGAGGGTGGATAGACTCGGATCTGGGTCAGTCTATAGGTTCCTGGGTCAGTCTAGGTCCCACTGAAGGAGCTAGAGGGTGGA
TAGACTCGGATCTGGGTCAGTCTATAGGTTCCTGGGTCAGTCTAGGTCCCACTGAAGGAGCTAGAGGGTGGATAGACTCGGATCTGG
GTCAGTCTATAGGTTCCACTGAAGGAGCTAGAGGGTGGATAGACTTGGATCTGGGTCAGTCTATAGGTTCCTGGCTCCGGTGGTTGA
ACTGGAAGTCTCGGGACAGGCACCTGTGGAAGGCAAACCTACTAAGGGTTCGTAACAGGCCTGGCTTAGGAAGACAAATGCCTGCCT
CTTTTGGCTGCCGATCCCAGGGGTCTGAAGCACCAGACACACAGGGGTGGGTGCTGACCCTTTGCCTCTCACCAGATAGGCTGAGGA
GCTCTGGCTACTCTATCCTAATAGGGGCAGGTGGGTTTTACAGGCTGTTAGCCACCGGGGTTTGAGTTGAGTCCCACGCTTCCCCTG
TTGGATGCCAGGGCTGCTCTGTGCCTTGGGATCCCTGATTCATCATGGTAACCTATGTGAGCTCTCCCCTTAACCCTGCCTGCTTAA
ATGCCCTGGTTTTGTTTTGGTAAGTGCTCTCCTTGGACGCTTACCCTGTGGGATTTGGGCGGTCTTTGGGGTGGTTGCTGCTCCCGT
TTCTCTCCATCAGCCCAGCCAGCAAGGGTCATAGGAGCCCAGCCCCACCTCCCTGATTCCTGTCAACGCCTCTGCCAGTTCCAGTTC
TCTGCTTCCCCCGAGCAGTGCTAATGACCCAGGGGACATGCTTCACCCACACTCAGCCTGGCCCAGCCTAGTGCTTGCTCACACAAC
ACTCGAGTCACCTACACTGAGGCCGCAAGTTCTCCATATATATATAGTTAAAGCCCTTTCTAGTGTTTTGTTTTATCTGATTTGGCT
TTGTGTGGCTTTCCTTCCTTTTGTACTAATGTTTCTCTCTGATCTTATTTGCATTCAAATTACCTCGCCAGGTGGTTCACCAATCAG
AGGTAAGAATGCTGTCCGTGCATTTCGCCACGCCACGCCACGTCACACCACGCCACACCACACCACTACTGTCGCCGTCGGTCACCT
CCACTCCATCCCGTCCAGTCCGTCACCTCCCCATACCCATCCTGCCCTCTCCAGTCTCTTCATCTCCAGCTCCAGCCCCTTCTGCCA
CCAGCCACCACACACCTCCACTCTCAGTCATTCCCTCCTGTGGACTCATCCATTATAAGTCTGCATTGTGGACATAACCAGCTGAGA
CCACCCAGGATGGCAAGCCCTGCTACTCAGGCTTTCTAGGCTAGTCCCACAGAACGGACCCATTTGGTTCCCATAGCGAATCCTAGC
CTGAACCTAAGGCTGGCATAGGCTGGGGAGCATGACAAGTCTGGCCCTTTCCCGTGAGAGTCTCCTTTTTCTTAGGATAGTTACCGC
TTGGCCTTTCCTGTAGTATTTTGGTCCCGTAGTCAGATGAGGATCATCCCTTATATCTCTTCAGCAGCAAGGACTAGGATCTTAGGT
GGCAATTGGGAAATAAACAGACCCATGTATAACTAGGTCAGCTGACCTGTCAGCTTGCAGCCCCCCAGAACTGCCTTCACACGACAG
GGTCCTGCAACCTACTGGTGAGAAAGCATAGATTCTGGGCTGATAGAGGTCAGGGTTCAAGTCTGCCTCCTCTTAGAACTAGAGCCA
GGACCACCTCCCCTCAGGCTGTGTGACCTACAGGAGACCCAGGACGCGTATGGACAGTTTGTCACATACAAAATGTAGTTTGTGTTT
CTATGCCACTATTTTCTTCCCATATTCGTCAATATTTGTGGGCACATCTTGGCCCTCTCTTGAAGGCTAGAGAGCTCAGGAGAGAAG
CTGGGGCTGGGGAGTAGCTATTTGTCCCTCTGACACCAGGACTCGGTTTCCCTTCCCTCTCTGCCTCTCCCTGCACATCCGTCTCCT
TAGCACACGCCCACCCTGCTCTCCCTGCTACTGGATCTGCGTGTCGGTACCAGATTTGGAGATATCTGTGTGCAGATAGATCCAGTG
TCTGTCCTGGGTAATGTCCTAGTTTCCTTTTTATTGCTATGATAAACACCATGACCACAGGTGACCTGGGGAGGACGGGGTTCATTT
GACCAGCATGTCCCAATTACTGTCTATCACTGAGGGAAGTCGGGGCACGGATTCAAACAAACAGAGAAAGCCAGTGGAGGAGAGCTA
CTTACTGACTTGCTCTCCTTGCCTTGCCCAGCTTGCTTTTTTCTCTACACCCAGGACTACCTGCCCAGGGGTGTTTCCACCCACAGT
GAGCCTGGCCCTCCTGCATCAATTGCTAATCAAGAAAACATCCCACAGACTTGTCTACCAGCAAATCTAACGGGGGCTTTTTTCTCA
ACTGAGAGTCCCTCTTCTCTCATGACTCTCATGGCATGTGTTAAGTTGATAAAAATAACTAACAAGCACAGGTCTCCTAAGCAGACC
TAGGCTCTTGTCCCAGGGGAGCTTCCTCCGTCCATCAGCTACAATCCTGGGCACTCTTTCCCTCAGCTTGTGGCACCAGCTGCCTGT
CTATCCCCTTACTTTTCTTGAGCTGACCATGGGCACACTCCACCAGCCAGGGCCCTGATCCTCACCTGTTTGAGTGAACGTCTGCTA
TAACGGTGTCCATTCATTTCATGAATATGACCCACTGGCTGCCAGTCAGCACCGTTGTTGCAGTTGCTGGGCATTTGCCTTGTGCTC
TGATGTCCATGGGGAGGCTGAGGTCAAGGCTACGGACAGCCAGCCCTTTTCCTCTGTGTTAGTGGCTCGCGCACACACTCACACACA
GAGCCACCACCACCACCCTCAGCCTTCTGATGTGTACAGCTCCTGCTGCCATGGTGCTGATGGTGGAAGGGGGAAGAACGGGGTTTG
AAGAAGCTGAAGCGTGGACCCAGAGTGTCTCCCCGTGGGCCTGGGCTGTGAGGACACAGCTTGAAATTGCCTCTGAGGTTCCTGTCC
AGTCCAGTTAGGTGGGACCTGGCAGGTTCTGTCTGTCCTCTGTGGGCGTGATTGTCAGTCGGGGTGGAAGCAGAAGCTCTTTAGAGC
TCTTCCAGCTGTTCGTGAAGGAGGTGGTTTTCTGTCAGACTGTGTCCTGTGGGGGTTATTAGCTGTCCGTTGGACTGTCCTGTGTGG
GTGTAATTGTCTGTCAAATTGTTCATGGGAGGGGCGTGGTGCTTTGCCCTGTGGGTTTCTCTGTCATAGATAGCTATCCGTTTGCTT
TCACTAGCAGTTTCCAAGTGTTTATTGCTGGGGGGTGGGCTGGCATCAATGCAAATGATCCTTCACCTTGGGGTCACTCACTTGACT
TGACATGGCAGTGTGGGTCCCAAATGCAAACTTCATCCATCCTCTCAGAGGGTCCTTTCAGCTACCCCCACCCCCAGCCTTGGCTCC
TGTCTGTCCGCCTGTGGCCAGGAGCAGCCCCTCAGGGCGCTGCCCCAAGTGGGAGCTACAGTCTCCATTAGCCTTGTGAATTATGCA
GCAGCCTTGGTGGGTCGAAGCACTTTAGCAAGACCAAGCCGAGGAAAGTGGCCGGAAAGGGTGGGGCTAGCAGGGTGGAGGCAGAGT
GGGAGACGATGGTGGGAGTGTGGGGCCGACGCTGCTGAGCGGCCAGCCCCAAGGCAAGCCCCTTCCTTCCTTCCGAGTAGATGATAG
TGTCTCAGGCTCTTGTTTGATGAGGACATTGCTTCTTTAGGAATTCCAGACCTATGGGGTCTGTAGGCTTTGAGGACAAGAACATTG
ATAAGCATGGTTATTGGCAAATAGCCTTAGGACTCACTCGAACAGTGGTGGACGCTCTATGGACAGCCCCTGGGTGGATCTTGCTTC
TAAGTACAAGTTCTTGACTCTTGCTTAGAGGGACCTGGCTCATCAGAAAGAACTTCCTGAAAGCCACACACAGGCCCTAGGCTGTGG
CCCAGGAGAGCCCCTGTCGCTGGGGCTACACTTGTTGTCGAGGCTGCCGTGTCCCAGGCCACACGCACCATGGACCGTGACCGACGT
CTTCTCTTGTGTCCCCCCCACAGGTGCATTGCAGATAGAGAGCAGCGAGGAGTCTGACCAAGGCAAGTACGAGTGTGTGGCCACCAA
CTCTGCAGGCACACGCTACTCGGCCCCCGCCAACCTGTATGTGCGAGGTAAGAACTTGGCCACACCCAGCTCTGTCAGCTTGGAGCC
AGGCGTCACCTGCCAAGCATTGCTGCCTAGAGGAGACCTTGGTGCAAATACTGGAGGGACTCCGAAAGTCTTTCCTTCCCATCTCTC
TCCGCAGCAGCACTAACAGCCACCACCTCCATAGCTCCCAGGGGCCATTCTACTAGCAGCCGCCACGACTTGCCTTCTTTCCCTTCA
GGCCCTTGGGGAAGCAGACGCTCCCTCCACCCTCTCACCCTTGGGAGCATTTGTATCTCTTGTAGGTCTTTGCTGCCCGGTCCCAGA
GCCCTATGGGAGTCTGGAGCCTTCTGAGCAGACATTGCTGTTCGTGTGTGTGTGTGTGTGTGTGTGTGTGTGTGTGTGTGTGTGTGT
GTGTGTTGGTGTGGTGGGGTGGGGAGTGGGCACACAGACACATGCAGTTACATGTGAATATGCATTTTTGACCTGACTTTCCCTCCT
GCTACTCCAGCAGGCTGGGCTCTAGGACTCTGGTTCTAAACTTTAAATCTGGCTCAGGAATGGCCCAGATCGGCCTTCCGGGAGGAG
CCTGCTGTGGTCTACGACCAACTCCGCCCCTCACGTTGCGTGAGGCTGAGGTAGCTGAGCCCCTATGGGTATATTGCCTTGAGCATT
CCTAGTTGGGGCGAAATGGACTGGGGGCTATTTACAGTATTTACAGTTCGGTCCACAGCTGAAGGGTCTGGGACTGTTTTGGTGTGT
CCAGCCTCTGTTTATAGCCAGAGGGAATCAAAGTTGGCCGCCTGGGCCTCCCTGAGACCATGAGAGATAGTCGAGAAAGATGGATCT
CTCACTGGGGATGGCTGAAGGAAAGAGACCAAGCTTCCTTGTAGCTCCCTGCAGTAGTCACTCCGTCCCTTAAAAGCTTCCTTCCCT
CTTTCTGAGGATAGTATGACGTCTCTCCAACTCATGGAGCCCAAGTGATGGCAGTGGCCTGTCCTGTCTCAAGGCAGTTCAGTAGAC
TTGCTTACCCAAGGTAGAGAGGTGCAAGCCACCAGCTGCTTACTGAGGGCCAGCCATGCCCCAGGGCACAGGTTTTGGAGGGGTCAA
AGGTGTCAGCTTGTTAGTTGACCAGAGGGGCTGTTTGTATTGGGCTAATTGTGTTCCTTCATGGGAGTTGGGAAACCCCTCTCTCTG
TCCTTGTTGCTGGGTGCCCTTGTATACCCATCAGTGAGATGACCCACGGCCAGTCTTGAGGAACACCACCAGAACAGAGACCAGGAA
CCCAAAAACTTCCTGAAGGCAGAGAGGAGTAGAACTGGAGAGAGCACACTTAACCCAGGGCTGATCCAGGCTGGGTATCTTAACAAA
GGCCCAGGAGTCTGCCAAGTCCCTTCTGGGAGATTAGCAGCTGAAAAGATTTCACAATGACTGGCCTCCTCCCCGCTCTTCAGCCAA
GGCAGCCCCTATCTACACATGGCTACAGCAGAGAGCGGAGAATCCAGCAGGGCCTTGGTAAACCCCAAGCCAGCTGGCTGTTTAAGC
ATTGCTTTATGGCCGTTTCCTAGGGTAGAGCTGCCAGAGGCAGTACCAAAGCAGGAAAAGGCCACCCAGATTCAGAAAGGATGACGC
GGGGCATCCTAGCTAGGCACCACACCTGTCCAGCTCGCACCGACATGCTGGCTGTGGCCAACGGCGGGGCCATGGTTATCTCTGGAA
ATGGACTGAGGTGGCTGCTAAGGCACTCCTTGGAGCATCCCTACCTGCAGAGTCCCCGCTGTCCACACATGATTGACTTGGACATGA
CCCTAGCTGGAGCCTGCCTTCTCCCACCTCGTGGCATAAGATCCACTTGGAAGAGCTTTGGGAGGTCAAGGGCATGGGAGCTTCTTT
ACCCATTTTACGCTCAGCCCTTCCTGCCTAGAGCCTAACGTCGCCAACCTGGCTTCTATAGCTCACCCTCCAGGCCCTGCCTCATAT
CTAGAACCCATCTAAATCTATGAGGGACCTTTGATTCTCGGATCTGTACCGTAAGGAAAGCCCTGCTCACTGAGACCCTGGCCTGTA
GTGTTTGCAGCTTCCTTTTGTGCCTAGCAAAGCCCCATCAGCCCCAGGAGTGCTCTGGGACCACCTTGTGTAGTAGTACTAACTACC
AGCTCTGTATTTGTAACTCTGTATGTGGGTGGCTAGGGATGGAAGAACCCCGCCCCACTCCAGTGCCTATCTGCTGGACTCTTTGTC
TCCCAATATAGCAATTTCAGTCCATAATGGTTCCTGTTATCACCCAGAGATGGAAGTCCCCCAACTCTTGTCAGGCCTTAAGCCTCC
TGATTCAGGCTAGTATGAATTATAGCTCAGATGTTTCTTACCCTTCGGACTGTTGACATTTTAGACTGAATACATCTCTGTTGAGAG
CCATTCTACGCATAGTAGGATATTTAGAAGCATCCCTAGCCAGTCAGGAACCAGTAGCAGCACTTAATAAGACCAGTCTAAATGGCC
ACAACTCTCTCCAGACGCTGGCACCCAGAGGCAGTTTGCCCCTGTGGATACCCTTTCTCGGGATTGCTGCCTCCTGAGGGCTTCTGT
CTCCATCTCTAACTTTGTTCTTCTGTCTAGTGACTCATCTTCTCAGACTTAGCCTCCCTCCTCACGTCCTTCCTCTGTTCCCCAGAC
TGTCGGGGCTCTCCCTGCCTCTCTGAGGCCTTAGGCTGCTTCAACCGATGGGGCAGTGCATGGGCATCAGAGCTTCCCGCATGCTGC
CAGGAGCACAGGAGGCGCGTGAGAGTCAGCTGTCTGGGCTCTGTTGGGCTAGCGCTGGGGCACACACATCGTGCAGTCACAGCTGAC
TTTATTCACAGTTGCCTGCAGTTCCACCCACCGTCTAGCAGGCCCAGGCACGCTGGGTGGACCAGCCTCTTTCTCCATCACCATCTG
ACTCCCTCCCTGAGTGACTCACTCTCTCCTTCCATCCACGGCGTCTCTCCCCTCCGGCTCTCAGGCATACAGCTTCCCGGGCCAAAG
GCAGAACTCTGCCCTTGGCAAAGCTTGCCTTACCTGTACCAAGATCACCAGCCTCTCCGTTCTCTTCAGGAACCCGGCATCTGAGAC
TCAGCTGTATGACTAGAGGACATTTCCTCTTAGCCTTTCTTTCCACAGGCATCCGGGCGTGCTCCTCACACTGGTGTTGGCGAGGCA
TGGGGCTTCCTCACAGTGGTGGGGCTGGAGTGGCTTGTGTGCCTCGGTGGGTTTGTGAGCTGGGTGGACCCTGACCCCAGAGCCTGG
TAGGGTCTGTCTGAGCGGTGAGGGTGTGTGAGGCATGGTGACTAGCCCGTGCCCCACTTCTACCCCTACCTGCTCTGCCCTCATCGT
CCCTGGCACTGCTGGTGGGCCCTACCCTGTCCACTGTGTCTCTCCATGAGTCCTAAGTCCTCTGTGAGCGGCGTGGTCCGTGTGCAC
CTGTGTGCACGTGTGTGTGCGAGGGGCACAGGAGTCCTGTCTTGTCTCTGTGCTGTGTGGGCAGATGGAGGTTGGCCTGTTTTTACT
CTCTCTGTGTTTCTCCTTGTCTTTTTTTATTCCCTCTCATCCTCATCGCACTCTGCCATCAACCCAAACTCTCATCTCTCAGATCAG
CGAGAAGGTTGGTCGTTTTCACTTCTTATCCATCTACAGTTCGCCCACCGACTGTGCCCATCAGTTGGGACCAGCGGGCTTGGTCGG
CTCCCTCAGGCTCACTCCAGCCGCGCCTGCCCGCCAGTTGGCCTCTTCTCGGTGTTTGGGCTGGCTGGCAGGCAGGACCAGGGATGG
GTGGGCAGGCCTTCTGCCCTGCATGTACCTGTTGCTTGACCCTGATTCGCTGTGTGTCTGCATGTCCCCTTAGCCAGGCTCCGCTGT
TGGAGTGGCCACACATGCACGGTGCACCGCATAAAACTGTACCCTACAGAACTGCACTCAGCTTTGCACCCAGACCAGGCTAGCCCT
CTGTTTCAGGAATGGTCATGGGGATCTCTGTCAGAGAAACTAAACTACATGCTTGGCATGTAGGAGTTCAGGGAAGCTTCTACCATT
TCCCCACCCACCCTTATGTTGTCTGCGGAACTAAGAGAAGGCAGAACTGCAGGAGGCAATAGGAGGAGGCTAATTTAGAGTGAGTTG
TCCCCTTGAGATCTCACAGTACGCGACACAGTCCTGCAGTGTCTGCCCTGCCCTTGAGGCCCCCAGAGCCTTACCAATGCTCCACAA
AGCAGACTGGCTGTACTGAGACTTAAGACCAGCCTTGGTCTGCCTCTCAAAGACCCTGATGGCCGTAGTCCAAGGCAGGTGGCATCA
TTTTCATACACTGAGTAAGTGTCCCGACATTATATCTTTTCTGTCTCCTACCAGGTCCAAACCAAGGTCACCCTGCCTGCCCAGACC
ACTCTACCCGAGCTTCCAAGCCAGCCTCTGTGCCTCTCCGAGGGTACAGTGTATGTGGCTCACTGACCAGCTAACAGCCCTCAGTCC
CATGCTGGGCAGTGGCACTGAGCGTTCAGATCCAGTTTGCTTATGCCTGGAGTGACTGAATTGTCACTGCAGTCTCCTTAGGCCTTG
AGTATCAATCAGTCCAGCTCAGCCTTAGATTGGGCAGGGCTTTTTGAAACTCCCTGGATCCTCTCCCAGAGCTGTCCCCTGGAGCCA
GGCCTCTATTTCCCAGTACTGGACCAACAAGGGGCTGGTGGTCCTTTATATATCGTGTGGTGTAAAGGGTTAATCACTGGGCTTTGG
GGGGTCCAAGGAAGAAGCACAGGGCAGACCCCAGCTCCCAGGTGGCCTCTGCATCATGCTCTGTGGCTTTGAGTTTACTCCGGGAGG
ACAAGGGAGCCAGTCAGGAGGAGGGCTGCAGCCTGGGCTCAGATATCAACATAAGCCTCCATCTGTATGCACTCCTGAGCTCTGGGG
CATTTCGGTTACACCATTTCGCTGACCAGGCGGATCTGCCAGGGCGTTCAGAAAATAGTCATCAGCAGACAGCCCAGCCCGCAGCCA
CTCCCCCCACACCCCTTCACCTACCCTCTGCATCCTTTGTTCTCTCCCCACCCACCCTGTGGTCCCACCAAAAATACACACATACAC
AAATATTTAACGGGAAGGAGAAATGAGTTTCTAAATATTCCGGGGGGATTTGCCGGGCTGGTAATTTGTTTGGTGCTGATAATTGCA
TCTTACATTTTTCTAGCTTTACTTAAAACTGTGTGCCGGGTGTGCCAGCATTTAATTACTGCTCTGCGGCAGCCACAAGGTTATTTA
TTAAAGAGTTATTTTATCTTGATACAGTGGATCCTGCCCCTTATCCCCTCCTTAATGTTGTGTTATTTTCATCAGAGAAATTTCCGC
AAGTGAATGCAGATTGCGGGGCTACCTCCCCCTGCGATTAGGCTGTCACTCCACTGAATGCGGATACCTCCAGGCGCCGCACCACCC
TATTATAGGGGGTTGTCAGCGCAAATAATTAGACTTAAAAGTTACAGCTGAAATATAATCCAGAAATGGCAGGGCCCTGTTTTGGAA
ATTGTCTATAAAATGTCAGCAGTAAGGATGCACGGGGAACAGTAATAGACCGGCATTGTTGGAGCCTGAGATTAGACCCTAAGTGCA
TTTTCCCCAGCTCCAGTTTTTCCTTCCCTGCTGTTGCTTCTGTCAATAGACCAAGTCCAGGGAGAGTCCTGTTCCCTTTGGAGGCCC
TGTGCTTGTGGCAGCCGGGGGAGGACGGTGGAGATGCTATGTTGGAGCATCAGCCACTTGCAACTGTTGGCACAGGAGTAGCTGTCT
AGGCTGGGCTAGGACACAGGGCCTCTAGCATTTGGGGCACTTTGTTGCCTCTTTCCCCATTTTCAGTAGATATGCTGGCCTACCTGA
GCTCTAGCAGATTGAACTTCCAGGAGTCTTTGCTGTGGCTGGACCCCAGTGCCCTTCTATAGGAATGTAGTTATCAGTGGAATACCT
GGGGCCTCTGGCTGAGTTCCTTTCCCAGTGCTGAGGTGACCCTGACCTCACACCTGTCTGTAGCCCCCAAAGTTCTCCCTATAGCTT
GCATCTTGGAGGGAAAATAAAAGCCATTCTTAGCCGGGCGTGGTGGCACACGCCTTTAATCCCAGCACTCAGGAGGCAAAGGCAGGG
GGATTTCTGAGTTCGAGGCCAGCCTGGTCTACAAAGTGAGTTCCAGGACAGCCAGGGCTATACAGAGAAACCCTGTCTCGAAAAACC
AAAAAAAAAGAAAAAGCCATTCTTAGTGTCACTTCCCATGGGGGCCCAGTTTCCTGACATCGACTAGAGATGGAGCCTTTGAAAGGA
GCGGCCCAGGGCATTGGCCTGGCTCCGAAGCTGATCCCTGAGGACTCTGCTGGCTTGGAAAACACTGTCCACCATGGACTATCCAGG
GGTCAAAGTTTGGACATGTTTAGGTATGGGCCCCAGGTATTTTAGCCAAAGACCTACTTCCTACTGCATAAACCCAGTGGCCCCTCT
TTCATTTGGGTTCCACCATTAACTAGAGCCACCATTAACTAGTGTCACTCTCAAAAGTCTTATCTGTATGCCATCTGGAGCTCGACA
TTATGCCAACGCTAAAGCCCCATGGCCTTCATGGGCTTTCGAAGATAGAGTTTTTACCACACAGACCTGATCTTCCTGAGGATATAA
AGGCAGATGCGTCACTTCCTGTGTCAGCATTGACTCTGGCCCTCATCTGCAATTGAGACATGGTGGCACAGGCACCCTCTGTACAGA
GTACAGAGTGGCTTTCCACTAGGCATGATGTGCATGGCTTTAATCCCAGCACTCAGAAGGCAGAGGCAGGTAGACCTTTGTGAGTTC
AAGGCCACCCTGGTGTATACACTAAGCTTCAGGATAGCCAGGGATATATAAACCCTGTCTTAAAAAAAAAAAAAGAGTGGCTTCATC
AGTACATGACTGCATGCCCTTCAATGAAAAGGAAATGACAGAGAGGCTTGTTCAGTTCCAGCAACTTCTGTGTTCTGAACTCCTCCA
TTCCTTGCTAGGCCTGGACCTGGCTTACATGCAGCACTGTATACCTGTTCTTCACCTTGGAGTGCTCAGCTATTAGACCTGGTATGA
AGCCCAGGAGTGCCCATGCCCTGGGCTGGCAAAGCCTGCCTGTACCATCTGCCAGTTGAAAGTTCTTGCAAGGGAGTCTGTTACCAT
GGGACACTTGGTAGCTATGATCCCTGGGATATACCTGTCCCTTGTCATGAAAACTGTGTCTGTGATACCGTGTCATCAGCCATGTAG
ACTCCTGATAGTAACATACATGATGCCCGGCAGTGGTGGCACACACCTTTAATCCCAGCACTTAGGAGGCAGAGCCAGGCAGATTTC
TGAGTTCAAGGCCAGCCTGGTTAACAGAGTGAGTTCCAGGACAGCCAGGGCTACACAGAGAAACCCTGTCTCAAAAAACCAACAACA
AAAAACAAAAACAAACAAACAAAAACGAAAATCCATACATGATAAAAAGGCTAGGCATGGTGTCATACACTTAATCCCAGCACTCAG
GAAGGGGGCTGAGCTGAAGGCCAGCCTGCTCTACAGAGTAAGTTTCAGGACAGCCAAGGGTATACAGAGAAATACTGTTTTGAACAA
CAACAAAAAGCATCAATGATGAGGGGTTTGGAGAGAAGGGTTAAGAGCATTTGCTCTTGAAGAGGTCTATGTTCAGTTTCCAGCACC
TACATAGTGGCTTCCAACCATCTGTAACTTCAGTTACAGAGTCTCTAGCATCCTCTTCTAGCCTTCGTGGGTTCCAGCATACATTTA
TGATTGTGCAGGCCTACATACACATACATTAAAAAAAAATTAGGGTCAGAGATTTGGTTGAATGGTTAAGACTACTTGTTCTTGCAA
AGGGCCCAGGCTCAATTCCCAGTTCCATCCTGAAAGCTCACCACATCCTTGGGTATCAGGCACCTAGAGTAATAATAAAATAAATCT
TTAAGTGAATAATAAAGTAAGTTTTGTTGGACAGTGGTGGCACAAACCTTTAATCCCTTCACTTGGGAGGCAGAGGCAGGTGGATTT
CTGAGCTCAAGGCCAACATGGTCTACAGAGTGAGTTCCAGAACAGCCAGGGCTACACAGAGAAATCCTGTCTCAAAAAACAAACAAG
AAATAGATACGTTTTTTTCAAAAGAGACTTAGGATAAACCCAGGTATGCTAGACTAAGCAAAATAAAGAGTCTTATGATACAAAACT
AGGAAGTGTAGAAATAGATTCGGTGTTGGGAAGCCCTGGAGCCAGGTCTTCAGTGTGTTCGGGCCGCTGCTCGCCATCGCCTCGCTC
TGTTCCTCCACTGCTCTTACCTTTGTACTGCAGCCGGTCCTCCCCGGCCTTAGGAAAGGAAACCCTGAGCTCAGCTGCCCCTGTAAA
GTGTGTTGCTGCTCTCACCGGCTGTGCAAACATCCAAAGGTTGCTTCTGGTGCAAAGGTTGTTACTTATTAGCCAGATCTAGATTAC
AAACCCCGTGCCAAGGATGGAGAGATGGCTCCCCTAGAGAAAACAAAGATGGGACTGGAAATGTTGCTCAGTGGTAGAGTACAACAC
CTAGCATATATGAGTCTCTGAGTCTTCTTCCTCTAAAAGAGAGAGGAAGAGAGGCCTTGGGGGGGGGCGGGGAGGAAAGAATGAGGG
TGCTGCTCTTTCAGGCCAAAGGTGGAAGTCTAGACATGTACAATGCACTCCAGGTAAATGTCTGCCGTGAGTTTAGGGAGCAGACCC
AGCCTGCCTTTCGCTGTGACTCACTTGGGGCTGCCAGTGTTGTGATCCCACCCAGGGCTTAGGGAACCTGGCATAGAAGGTGTGGAA
GCAAGTTCTACCCACGGTCCTGTAGGCTGGCCACGGAGCATCAGGAACCACACAGAAGCTTGTTAATCAGGGGCCGCCAGTTGTCTG
TGCAGAGCTTGGGAGTTGGTTAGGGTGAGACGTTAGAGGGAGCGCAGGAGTGGTGTTATTTAGATGTTACTAGTAGGGGTGATGGAT
TTTTTTTTTGTACCCCATTTTTTAATAGGGTTATGTGATTTTTCTGGAGTCCAGCTTCTTTTTTGTTGTTGTTGTTTTGTTTTGTTG
TTTTTCTTTTTTGTTGTTGTTGCTTTTTTTTTTTAAATTGGGTATTTTCTTCATTTACATTTCAAATACTATCCCAAANNNNNNNNN
NNNNNNNNNNNCCCTCCTCCCCTACCCACCCACTCCCACTTCTTGGCCCTGGTGTTCCCCTGTACTGGGGTATATAAAGTTTGCTAG
ACCCAAGGGCCTCTCTTCCCAATGATGGCCGACTAGGCCATCTTCTGCTACATATGCAGCTAGAGACATGAGCTCCGGGGGGTACTG
GTTAGTTCATACTGTTGTTCCACCTATAGGGTTGCAGACCCCTTCAGCTTTTGGGTACTTTCTCTAGCTCCTCCATTGGGGGCCCTG
TGTTCCATCCAATAGATGACTGTGAGCATCCACTTCTGTATTTGCCAGGTACCAGCATAGCCTCACAGGAGACAGCTATATCAGGGT
CCTTTCAGCAAAATCTTGCTGGCATATGCAATAGTGTCTGCGTTTGGTGGCTGATTATGGGATGGACCTCTGGGTGGGGCAGTCTCT
GGATGGTCCGTCCTTAGGGTGATGGATTTTGAAAGCTGTCTTAGGCAGCCCTTAGTACAACCCCGGTGACTTTTACTACATGTCTCA
GCTCTAAGCCTCAGTCTGACCCCTAGGAATTGAGGTGAGATTGAACCTTGGGTTCTTTCTTGGGATGCCACCTCTGCTTCTGTGACC
TGGGACTGCGTGACATGCTTCTTACAATGGGTCCTCTGCATGTCCGGACCCCCCAGCAAGAGTCCTCAGAGACCTGTGCTCTCATAG
TGGCCCTCAGGTCACTTGGGCTTGCTGCCTGTCCTCTCTGGCCCTTTGTGTAGGGTGGCCGTCTAGGATACACATGAGCCTCTGCCA
GCAGCTGCATGACCTGCCTGTGTGCCAAGCATGTCTCTGTGTAGCTAGCTCTTGTCCACCTTACCCTTTCTGCATGCTCTGAGTGTC
TGTGCCAAGCTTGCCACTTCGGGGTTGGTTTCCGGCTTTGCCCTGGACTGAGAAGCCCAGGACAGCTTCTGGCCAGGGGAGTCCAAG
GCTGAGGGTTGCGGCTAGGCACAGCCACCTCCCTTGAGAGCTTTACTCACTGGTGCAGAAAGCCGTCTGCTCGGCCCCCTCATCATG
TTACTGCCATCCTCATGCCCATCCTGCCCGCGGACCACGTCCCTGGCCCGCCCAGCCCCTTGTCCCCGGACTAGCCCTCCTAAGGCG
CCATCCACATTGTGGGTCCCAGGCTGACCTGCATAGGCAAGGCCCAGCAGGTACTTGTGCCATTTTCTCTTCTCTCCTGGGGCCATG
GCCTAGAGGTGGAGACACTGACTCCAGAGAAGGTTGCCATGGTACTCCCAGACTGATCCTTTGCCCCACCCCCCACCCCCAGCCATG
CTCTGAGGACCTGGATTGCTTTCATTGATGGTCCCTCTGGAGACACAGGAGGAGGGGAGTCAGGAATAAACAAGGGTGGTCATTGGA
GGACCACCTGGAGTGTGGCCCTAACCAGGCCTGGCTCTTGCATTAAGTCCTGTGTGACCTTGGCTGAGTGTCCCATGCCATAAGATG
GGAGCTTGGTTATGCAAGTTGTCATTAAGAGGTGTTCCAGATGGTCCCTGAGTGCAGGGACGACTTCTGGAAAGTGAGGTGGGAAAT
GGTGAGCCAGGCCCCAGGGACGCCACTCCTCCTGTCTCTGAGCATGGGCTTGGCTCTGACAAGAGAGGCTTCCTTGATGAGGGGCCC
CTGCCCTTCTGTCCACCCTTGCATCCCGCCCAGCCTGTGAGTGCCTCTCTCCCTCCTCCCACAGTGCGTCGCGTGGCTCCTCGTTTC
TCCATCCCTCCCAGCAACCAAGAGGTGATGCCGGGCGGCAGCGTGAATCTCACATGTGTGGCAGTGGGCGCGCCCATGCCGTATGTG
AAATGGATGATGGGCGCCGAGGAACTGACCAAAGAGGATGAGATGCCAGTTGGCCGAAATGTTCTGGAGCTCAGCAATGTCATGCGA
TCTGCCAACTATACCTGTGTGGCCATCTCTTCATTAGGCATGATAGAAGCCACCGCCCAGGTCACAGTAAAAGGTGAGTGTAGCGGT
GCCTCAGCTAGCTGATGTTCTCCTCTGTCTTGGGCTTTAGATCCTCAGGCCTGTGGCAGATAAGGAGGAGCTGGCCTTGAACTCATA
GAGCTCTTCCTGCCTCTGCTCACCAAGAACTGGGGACACAGCTCAGTTGATAGAGAGCATTTACCATGTTAGAAGCCCTGGATTCAG
TCCCCGGTACTGCCAAGAGGAAATCAGGCTGTAAGCTAGGATTAATCATGTCACTCTTCCTGGTCCTCTTTGGGAAGGAGCCCAGGG
CCTCACACACACTAGAGAAGCGCCTACACTCTGAGCCTTACACTGGTTCCTTTATAAAAGCAAAAGGTAGCTTTCAGGGGCTCACGT
TGCTCTGGGAGTCACCCAGAAACATTAGCCGGTCTCCCACTCTCAGTTTCCGCCAGCTACACTAAAACACACAGTTCTAGTCCCCTG
CCTGCCCCTCCTCTATCACAGTAACTGGCTCAGCACTGTGAGACTCTTCCCGTCCCCCAAGCCCCACACCCATCCAAGGAGAGGCTG
TGCTTTCCTCCTGTCCTTGTTCACAGTTACCATCAACTCATTTGCTGATTATTTTCCCAGACGATAAATTCCAGAGAGCAGGAATGT
GACCACGAGCCATTGTGGCACCAAATGGGGACTTAGTGGCTATTCATTCTTCATGCAGCGATCGCAGGCCTGGGATTGTGCCACCTT
TGTTTTGAGCCAAGTCATGGGGCCTAGTGGCGGGGCTAGCAAGTTCATGGCTCTTTCTGAACTCTAAAGGCCAATTCCTTGGGAGAT
CGTGCATGCGTGTGTGCGTGTGTTACACTTGGTACAAGTGAGAGTAGATGAGCAACAGGTAGGTCTCTGCCTAGTGTAAGCATGTGA
ACTGTGTGAGACTTGATAGGACCAGGTCTGTTTGTTGGGTGAATCACATGTGCTAATGTGACGATCCAGTGCGGGTGTGTGTGTGTG
TGTGTGTGTGTGTGTGTGTGTGTAGGTTCTAAGGGTGATGTGTTACACACCAAAGGTCTTAAGACTCCTTGGACCACCATCAGACCT
GGCTGCACTATCTCAGTAGTTGCCCTAAGAAGTCACCACAAGCTTTTCAAGCCTTTTTTGAAGGAGGGTTGTGGGCATCAACCACAC
TTAGGTGGCCATACCCTGTGTTAGGATGATGCCTTGGGTTTGTCCTCAGAATCATGATATCTAACAATTCTTTCTCCTCAGCTCTGC
CAAAGCCTCCAATTGATCTTGTGGTGACAGAGACAACCGCCACCAGTGTTACTCTGACATGGGACTCTGGAAATACCGAGCCTGTGT
CCTTCTACGGCATCCAGTACCGCGCAGCTGGCACAGACGGCCCCTTCCAGGAAGTGGATGGTGTGGCCAGCACCCGATACAGCATTG
GTGGCCTCAGCCCCTTTTCGGAATATGCTTTCCGTGTGTTGGCTGTTAACAGCATTGGGCGAGGACCACCCAGTGAGGCAGTGCGAG
CACGCACGGGGGAGCAGGCACCCTCCAGCCCTCCGCGCCGTGTGCAGGCTCGAATGCTTAGCGCCAGCACCATGCTGGTCCAGTGGG
AACCACCTGAAGAGCCCAATGGCCTGGTACGGGGATATCGCGTCTACTATACCCCAGATTCCCGACGCCCTTTGAGCGCCTGGCACA
AGCATAACACTGACGCAGGACTCCTTACCACCGTGGGCAGCCTGCTACCTGGCATCACCTACAGCCTCCGAGTCCTTGCCTTCACTG
CTGTGGGTGACGGCCCACCCAGCCCCACCATCCAAGTCAAGACACAGCAGGGAGGTAGGTAGGGGCTTTGTTCTAGATGGACAGCTG
ACATCAGAGGCTCCGGCAGAGTCCCACCTGTTTCCTCAGGCTGTGAAGTTTGGAGCTTGGGAGGGGCGAGGGTGTCATACTCCACAG
ATAGGTGGTCACTGGGGATGCACGCCAGGCTGGTGAGAGCTTGGCACCCTCTCACTGGGCCCTTGGAGGTGTTGCGAGGCTGACCTT
GCCTGGGGTGGACATTGCAGTGCCTGCACAGCCTGCGGACTTCCAGGCTAACGCTGAGTCGGACACCAGGATCCAGCTCTCGTGGCT
GCTGCCCCCGCAGGAGCGGATCGTCAAGTATGAGCTGGTGTACTGGGCAGCCCAGGATGAGGGCCAGCAGGTGAGCCAGGAGGCCAG
GAAGGGTGGAAGGGGGATAGCCCCCAGTTCCTGGTGGTCTGTGATGGGCACACCTAGGCAGGCTGCAGACCCTAGGTTTCCCTACAG
CTTTGTAGCTCTCACAGCCTATGTGGTCTGCACCACCCCTGTGCTAGAGGCAAAACTTCTAAAGTTTGCCTTTGTTCTCTTGGGTTA
GTGGCCCGTGTGATACTGTCCTAGCCTTGAAGCTTCCCCTGGGGACACAGTTCCCTGGTGGCCTTTGTGATCTTTGAGGTGGCTGTG
GCATGAGTCACGCCATGCATTTCTTGCAAGGCTGTGTGACTATGTGGGGTGGGGCTGTCAGAGAACCACAGTGGGTCCTCTGCCAGC
TGCTTGCAGCCCACGTGGGGATAACCTCTGAGTGGCCTTTGAGATCTGAGCAGTCAGTAAGTGNNNNNNNNNNNNNNNNNNNNNCAC
CGTAGAGGCCCGTACAGCACAGTCCAGTAAGTGTCTGCTAAGGCCACCCTTGTCACATCTGGGCTGGTACACTACACACGGGCACAT
ACCATTCCCTTTCAAGCAGATGGGTGGTCAGGTCTGCTGAGTCCAGTATTGGACTCCAGTCTGGGCTCTTGAGTCTGAACCCCGAGA
AAGGGACAGGTAAGTGTCCTGTTAGTCTGTGAACAGGCAGTGGTTGCCTTCTCTGCTCCCTGCCCAGCCTTCTGACCCTCTCTTACC
CCTGTGACTCTTATGTCCTGCACAGGTAAGTTCTGTGTGGCTGTGACTCATCTCTATCCTCTGTCCTTCTCCTTCCATACCTAGATC
ATAGCACTGATTTGGTGGGCCTGCCACGTTTGAGACCTGCCTAGATGCCTGTCTTCCTCCTCATGGTCTCACATCTCATCACCCATG
TTCCCCACCTCATACCCCACCACCACAGTTCTTTGGGAGTTGTGTAATCTCTGAGGCCTCTGCACTTGCGGACAGGTCCATGAGAAT
GTGACCGTGTGCCAGTGTGTGTGTGTCCCCACATCTGCCTGTGGGAGCACATGGCTTCTGTGACAACGTGACTGTTTGTAGGACCAC
CTGGAGTTTGTGTGAAGACCCAGCCAATAGCACCTGAGTGTGTCAGTGTGTGTGGGCGTGACTCTGTCGCACCCAGATTGTGATTGG
CACAAGATGGCAGATTGTAAATGTGGCTTCGTGTGATAGTAGACTAAATTCATTCTAGAAGCCTATGCTCTAGGTCTGGTAGCGCAG
CCAGAAGGCAAGGCCAGTATCCATAGGGCTCCTGCAGTGGACTGAGACAAACAGTGGTGCCTGTAATTACCCAAATGAGCTCTTAAT
TACATTTGTGCAGAGTGAGCTGGGAGGGTGGAGAGATGGCAGGCAGTACAGGAGCTACTGTTGTTGGTGCAAGGTCAGAGGCAAACA
CTGTTGGTGCAAGGTCAGAGAGAGTTCAGTCTCAGAGCTATGACACGTGACCTCCACTGAAGCTAATTAGGTAAAAAGAACAGGGAA
TATGGTGGCCCTGGGTAGGGAATATAGTGGCCCTGGGTAGGGACTCTGCAGCCACCCAAGCTGTACCAGTCGGGTAGCTCTGAAAAT
GGGCAGGTGACCCAAGATGCAGAGACCACTTAGAAACCTCTTTTGTGGACTAGGCGAAGTTTGCTGCCTTGGCTGAACTGGTCAGGG
GGTGCGGGAGGGTTACCTAGGCTACTTCCTGGTCTGGGCTTGGTGATAGATTGCAGATGCGGAGCCGTGGCTTAGAGATATGGAGGT
GCATCATGTTCCTGATAGAAGGCTCCACCAGTGCGCGCTCCCCCCAAGTCAGGTGATGCATGGAAGCAGGCTCGAGCAGTGCGCCTG
GTTTCAGGCACCCAGATTCAATCGCTGTGTCTGTCAGCGGTAGAAGTGTGGAGGTGTAGGGCCTGAGCTGGAGGAGGCAGTCCTGAG
AAGACTAGAAATGAGATCTAGGGCTGGGGAAGAGGACAGAGAGAAAGACAGACGTGGGCCTGAAGTTGCTGCTGAGTGGCTGAAAGG
GACTAGAAGTAATCAGAAAGCAGGCTTGGGGGTGGGGTGGTGGTGGCAGGCTGTGTGGTAGCTTGGCAGGGGCTCGGAGTGGATGAG
GAAGTAAGCCAGCATGCTAAGATGTCATTAGGAGCTAGGTGGGGCGCTGTAGCAATGAGGATGGGGTCTTCTTTGAAGGCGGGAACT
CTTGAAGTGCGTCCGTATGCTTTTGTGAGTTAGGAGGACCACGCTGGAAGCATGGATGGAGAGGGACAGCTGGGAAAGTCTCTGGAG
GGAGAAGGAAGGGAGCAGAATGTAGGTGAGAAAGAGCTCGGCCTTGGACTCCACTCTTGAGATTGTACAGGAAGAGGGGGGCTGGGG
CTGGAAATAGAAGCGGGTACTGTTGGAAGGCAGGCAGGCAGCAAGCTTATATGCCTACAGGTAAAGGGAGCTGGGGGTCTGGAAGCT
GTGCAGATTGACTGCGTTATTTGTAAAGTGGCAGAGTGAGCAAGATAGAGTAGATCTCAGCAGGTAGCCCAGAGAGCCCAGGGGGAG
CCCGCTGGAGATGCCTTGTCTGCCTGCCTCAGGGCTGCAGTATCCATTACCTAGTGTGGCTCAAGAGATGGTGGGAGAAGGAGGGGC
TGAGCGGATGGCTCAGCAGGTAAGAGCGCTTGCTGCGTAATCATGAAGAGTTGAGTTCAAATCCTCGGCACCCACATAAACAATAGG
GTATCGTCATATATGCACCTTAGCACTAGGACTGTTGGGAGGCCGAGTCAGGAATCTGTGGGGCAGGTGCCCATACTCACATATACA
TACACTACACACACTTACAGAGATGAGAAGTGAGTTTAGGGCACTGGTGAGAATGGTAAGCCAGGGTGCTTTTAAGTCACGTCTGAA
TATTCAGGGCTGTAAGGATGGAAGTAAGTGATGGGATCCCCAGGAGTAAGAGTAGAATGCTGGGGCCCAGGGAGACCCAACTGGGAC
AAGAGGTGGGCGCACAGGGTAACGTTAAAGGTCATTCCCAGTACTGGTGTGGTGCTTAGCCACCGGTGAGTCAAAGGCTGTTGAAGT
TACCCAGCCAGTGAGTGGCAAACTTGAGGTAAACGGAAGCCCCTGGATTGGTAGTGGCATGGTGTCACCAGGCACAAGGAGAGCACA
TACTTGCCTCCTAACTCATAAGGAATCTGTTGAATACTGCCATCCCTGTGACTAGCCGCAGACGTTGATAGCTCAAGCACAGAGCTC
TGTGCATGCAGGCTCAGGCTAAGTAGTCTTAAACCAAGGGAGGCCTTTGCATCACGTGTCCCAGAAGAGAATTATATTCTCCAAGTG
TTCTGGTGTGTGTGTGTGTGTGTGTGTGTGTGTATACTTGATACCATGTTTGTGTGAGACGGCATATGCATGGGAAAGACGCTGTTA
GAACTCCATATGTGCGTGTGAGACACTGCACATACTGTGTGTGTGTGTGTGTGTTTAGAAAGCTAGTTCTGAGGCAGTTAGGCTGTC
TCAGCAGCCATATGCCTAGAGGGCCTCAGCATGGCTGGGTGAGTCTCCAGTCCCTTGTTACTTGGTCACTGTGCCTGTGGTCCCCAC
CCTCCATTTGCCTGCTTTCCCCCCATTTGTCTTCCCCAGCCCCATCAGCCCCTCCCCAGAAGGTGACATGTGTGAGCACGGGCTCCA
CCACGGTCCGGGTAAGTTGGGTTCCACCGCCGGCCGACAGCCGCAACGGCATTATCACCCAGTACTCCGTGGCCTATGAGGCACTGG
ACGGCGAAGACCGTAAGCGACATGTGGTGGATGGCATCAGCCGTGAGCATTCCAGCTGGGACCTGCTGGGCCTGGAGAAGTGGACGG
AGTACCGGGTGTGGGTGCGGGCACACACAGATGTGGGCCCTGGCCCTGAGAGCAGCCCGGTGCTGGTGCGCACCGATGAGGACGGTA
GGCTGCAGTGGGTAACATTGTTATGAGGAGGGGTGGGGATGAGAAGCATGCCACCTGAGGCTGTCTGCCAAGCACCCAAGATCCCTC
CACAGGACCTGACAGACTCATTTTCTAAATTTAGGCTTAATGGTCTAAACTCTGGGGCGATGTAGTTGGTGGGGTATCCCGTATCCA
CGTCCTGGCTTCTGAGCCGTCTCCAAGATAACTGAAGCTTTAAAACCGTACAGGGCTGTGGCAGAAGCAGCAGCCCCACCTAATATG
AGAGCCAGGATTGAGAAGATGAGTGCAAAAGACTCAAGTGGGCTTTGTCTGGATACTAATGAAATTTGTTACAGATGGTGTGAGCCT
AGGTAGTTGAAGGTGTGGTACCTCACTTCCAGCTGTTCCCCACATCTATGTTTCCCATGAATAAGTAGCCCTGCTAGAGGGACCCAT
AGGGATGGCCCTCGGCAGGCAAATCAGCCAGCCAAAGAAACAACATTGGAACCTTTCCCAAGGGGGCAGGTGCAGAGTCACGGCCCT
AAAGTTAGCTGTACGTGCCTAAGCCAGGCGCTGTTGCTCCATATCTGACTCTCTTCTCTGCCTGGCCCCTAGTGCCTAGCGGGCCAC
CACGGAAGGTAGAGGTTGAGCCTCTGAACTCCACTGCTGTGCATGTCTCCTGGAAGCTGCCCGTCCCCAACAAGCAGCACGGACAGA
TTCGTGGCTACCAGGTCACCTATGTGCGGTTGGAGAATGGTGAGCCCCGAGGCCAACCCATCATCCAGGATGTCATGCTGGCTGAGG
CTCAGGTATGTCATTGACGGTGCTAGGTGGCAGGGTAAACAGTGACCAGTGTGTCACAGTGACCCCCTAGAGCCCAGATTTGTCCCT
CCAATGCCTGCCCTCAGAGGTGGTTATGCCTTTTATGGTCATTGGAGCTGCCTTGGACTGTCTGGGCAGGAGTTATTGAGATCGGGA
GGTGAGGCTCATGTTCCAGAGACTTTGTTCTTCTGTTAGGATTGTTAAGCTGTGCTAACCTAGGTCCTGCTACCCCCTCCACCCAGG
TAGCAGTCTTAGTTGCCAGTGCTCACGGGGCTGCTCCCAAAGACCCCCAGGCTCCTTGCACAGCACACTGGCTTCATCCTTGTGGTT
TGCAGAAAGCACATTTGCTGTGGCTTTGAGCTGAACTGCTGTCCCAGGCAATACTGTTCTTAGCATCACCTGGACTGCACAGCCAGG
GGCAGACAAGACTTTTCTCCATCCTCCATGGCCTCAAGCCAGTGACTGAGGCAAAGCTCTGCAAATAATGACCTGAGTGTCACTCTG
CTCTTGACCTGAGTGTTTCTCTCCAGGGCTGGACCTGCCAGCCCTTTTCTGTTGGAAGCTTCTGTCTTCCTGGGGCATCTGCCCACA
GGCTTACAGTGAGGTCAGTATTCTGACCCCCACCTTGCTAGTCCATCAAGGAGCACCGAATACTGTGAGATTATATGTGTGGCCAGA
ATGAGCCAGCATGTAGGTGGCAGGGATTGGAGAGGAACCAAGTCTCTACCCTGGGAAAGGACTAATCAAGTATAGGAAAAACTGTGT
TGTCTGTCCTTGGTCTCCCTGTCTAGAACCAGGTCCTGATGATTCTTGGGCCAGCTTCAAGTCCCAACAGAAAGATCCTAGATTCCC
TCCCCAACCAAGTGGGAACCCCATATTCTGCAGGCTGTGCTGAGGGATGAACCCCCACACAGACAAACTGCTTTCATACCTTTGGCT
AAGCTCCACCCTCTGAGCCAAAATTCTTTTCTACCCAGAGGGTACCCTCTTTCTTCTCCCAACCTGTCTATGCTGAAGGACCCAAGG
GTGGGGGAGGTGGGTGTGTTCATCAGCCTGCATTCCTGGGGCAGGTTGAGGGCTCCTCACAAAGTCTCAGCAGACACACCCGCTCAG
AAACATCCAGAGAATGACTCCATCCAGCCCCTCCCATCCTAGGAGTCTGGCGCCACAGCTGCAGCCTCTGTGGTTTCAGGAAAGTCA
CTTCGAGTACTTTTGTGTGCTGGTCCCCAAGAGTTTGAAGGGGGTTGGGTAATAAGACCAGGGACATTGTCCCCATATTTGTGTTCT
TTACAAGGAATATGCCCAAGCAGGCTCCAGGGAGGCCACGGAAGACAAGGCCTGCCCTTGTAGCCTTCCACCCCCTGGGACTTTGTC
ATTCTGCTTTGGGCCAGTGGGAGACCAGATGGTGCACTTATCCAGCCTGGCCCCAGGGCCTAGGTATCCCCTGTGTGACTCTTTTCA
AGGACACCCCAGCCTGCCCTTATCCACCCAATAGAACCATCTGTGCAGCCCTGGGCCCCAGTAAGATCAGAAAGAAGCATGTGGTGT
GTATCCCCACCAGATGTGCATAGCCCAGCCTGCCTCCCCTGTGCTCCCTCTAGATGAGACTCGCCACCCTCCGCCCCCTTTTTGTCC
CTTCCTTTTGTCTTTTCTCTCACTGTAGGAGTTTAGTCTAGCTACTCTCCATCCCCTTTTCCTTCTCTCCTCTCCCTACTCCTCTTA
CAGCCTCTTCCTTTCCCACCTACCCCTCTGTCTTTTTCCTCTCACTACCTGACATAGGAGGGGCTACCTGCGATAGCTTCACACCAG
GGCTCCCCTCAAGTAAGGAATCCTAACTAGACCTGGTTCCAGCCCGCTCTGATAATTCCAGGACTCTATAGCCTCTCTATATTCTGT
CGCAAAGACTGTGACAGAACTGTCTCAAATAGCAGGAGCTCAGGCCACTCCAGCACCAGGCTAGTGGACAGGCCAGACCAGCTTATA
TGAGGGTGATCTGGCTAGAGGACTCCAAACAGGGCCTGTCCCTCACTAGCCAACTCCTAGTTTCAAGACCACAAGGCTTCACATTAG
AGTCCTCACTCCATATACCTGTTCACCCAGCATACCAGTGCACGCTGGCATTAGAATTTACCACTGCCAGCCAAGAAACTAGTTTTC
CTATTTTGACTCCAGGCACCTGTAGAATCGAAATCAGGGGTGTGGGGTCTCCTTGTGGCTTTGGGGTAATGCCCAGGCTCTGAGCTG
GCTACTTACCCTTTATTATCTCTCTGGCAAGCCTGACTCTTGGCCCACTCCAGCGGTAAGTCCAGCACTTGTGTTCTAGGACACTCA
GCTCAGGTGACTCTAATTTAGTGTCTCAGCTGAAAGCAGGATGCATGGATGGATGGATAAGTGGATGGATGGATAGGTAGGTAGAAA
GAGAGAGAGAGAGAGAGAGGTAGGTAGGTAGAAAGATAATATAGATAGACAGAGGAAGAGAGGGAGGTAGATAGATAGATAGATAGA
TAGATAGATAGATAGATAGATAGATAGGGGGAGCAAGCTGAACAACCATAGCAGGACATTGGGACTCTAAGCACATATCTTCCCTCT
GCCTAAGCTGTCAAGTCTATCTCTACATCTGACAGTCCACCATGTCTCCCTGCCCTCGACAGGCCTGGTGCACACAATTGGTGCTCA
ATGATGCAGGAACTTGTGTCTTTGAGTGGGCACTGGTCCTGGGCTATCACCTCAGTGGGTACTTCAGAGCTATCTATCAGTTCTCCA
TGGGCCAGGTCCTCTCCAGGCCAAATCCTTCACCATCCTGTCTCCCTTTTTCTCCTTCTCCCGCTGTCAGTGGAGACCAGAGGAGTC
CGAGGACTATGTAAGTAACAGGTGTGCAAGCACGGGCAAGACTGGGTCAGACTGGGCTCATGGGACACTCCCCCTCTCCCAGCCTGT
GTTGCCGAGATCCACAGGGCACCAGCCACATCCAGAGAAAATTGGCGTGTAGACATGAGCACCAGGCTGAGCAGTGATTGGCGTTTC
CTCTAATCCGTACTCCTCGCCTGTCGAGCAGCAATTTGAGGCCAATGTTCACAATCGACCAGGACCTGGCCCCTGCTCTGGCCAGAC
GGGGATGGAGTTAAATCCAATCCCGATCGTTAGGCCTTATTGATCCCTAGAGTGAAAAATCACCTGCTCAGGCCCAGGCTGGGAGAA
GCACTGGGAGCCAGGTCCAACACAGGCACTGCTGGAGCCCGAGCCCTGGGAGACCCTCCAGCTTGGCCCTGCAGAGGGTTGTTGGCA
TCTGCAGTTGGCCCCACCTGAGGCTTAGAGCTGGAGGGACACCAGGGCTGGTGCTGACAGCCCTATGCCTACTCCATGGTGGTACTC
TCCCCTCCTCCTCCTCTCTGTGTGGCGTGGCTTGGTCTCCTATGGTGTTTCTCCGCATGTCCGGAAACATTGCCTTTGCTGGATGGG
TGTGTGGGTAGGATCCGCCAAGGTAGAAGGAAGTGGAGTGGCCGACTTCTTGGGCATCGGTGGAAACAAGAGGGGAATGTGTTCCTG
GGTTTGGGGAATTCTGTGTCCTGTCTTTTTTTTCCCTCTTATGTGGTCTCAGGAGGCATTGGATATCCCAGTCACCGGAGTAGATAG
GGTCACTGTAACTTACAGTGTTGTAGGTTGGGCCCTCTCCTTTACCCGGCTCTTTTATAAGTGGCCTTGCACCAGCTAATTCCAGAG
GAATGGTGGCCTGTATGCATTTGCGTGCCAAGAGGCATGGAAGGTACCTGGTGCATGCAAGGCTCTGTCTGTGTGTAGAGACCTCAT
GAGGTGTCACTCACAGCACAATGGCATGAAAGTAACAGTGTGGTCCTTTGTTCTTCTCTGAAACAAAGAATGAGCTATCTGCCCCAG
GCCTGGGGTTTTGGGTTGGGTGGCATGCTCCTAGCCCACGTCCCACCTGGCTGTCCTGCCTCCAGCAAAGGAAGCAGAGGAAGGATT
TGTCCAGCTTCACCTCCCAGGACCCTGGTCCTGTGTACCTGGGGCACCCTAGGCTCCAGTCTTCAGGTGACTGGTAGTCTAACTTCT
TACGCAGTATGATGGGACCTGCCTTTCCCCTTCTTCAGGAGACCACCATCAGCGGCCTCACCCCCGAGACCACCTACTCCATCACCG
TCGCTGCCTACACCACTAAGGGGGATGGTGCCCGAAGCAAGCCCAAGGTGGTTACTACAACAGGGGCAGGTGAGTGAGGGGTTCATG
GACAAAGCTGGGTGGGCAGCAGGGAGGATGGCACCAGAGCCCAGTGCGTGCTGTTCAGTCCCAGGACGGCCCACCATGATGGTCAGC
ACCACGGCCATGCACACCGCGCTGCTGCAGTGGCACCCACCCAAGGAGCTGCCTGGAGAGCTGCTGGGCTACCGTCTCCAATACCGG
CGGGCCGACGAGGCGCGGCCCAACACCATAGACTTTGGCAAGGATGACCAACATTTTACAGTCACCGGCCTGCACAAAGGGGCCACC
TACGTCTTCCGGCTCGCTGCCAAGAACCGGGCTGGCCCAGGAGAAGAGTTTGAGAAAGAGATCACCACCCCCGAGGATGTGCCCAGT
GGCTTTCCTCAAAATCTTCGAGTGACAGGACTGACCACATCTACCACGGAACTGACCTGGGACCCGCCGGTGCTGGCGGAGAGGAAT
GGTCACATCACCAACTATACTGTGGTCTACCGTGACATCAACAGCCAGCTTGAACTACAGAATGTCACCAACGACACACACCTTACA
CTCTTGGGCCTTAAACCGGACACCACTTATGACATCAAGGTCCGTCCACATACCAGCAAAGGCGCCGGCCCTCTCAGCCCCAGCATC
CAGTCCCGGACCATGCCCGTGGAGCAAGGTGTGTGCTGTGATCATGGCATTGGCTGCCCAAGCGTGACTGCTCCTAGGGACCTGTGC
TGTCCTTGACCCACTGACCTCTCGCAGCTGATCCACCAGCTTCTGCTGTGTGACACTTCACGCTCTCATGACCACTAACCTCTAATG
GACAGGCACCACATCCTTGGGTGTGTGGCCCTGACCTCTGCTGTCCTTGACCTACTGACCTATCTGCGTGGCCTTCCAATCTCTTGT
GATCTTGATCCACTGATCTCTTTCTGCTACATCACCTTCTAGGGTGTGCAGCTCTCTCTACTTTGCTCTGTAGCACTAACCTTTTGG
GGCCTAACCTACTGACATCCAGAGTCCGTGTGGCCACGTACATCTTGTGACCACAACCCACTAATCCCTGGCTACTCTGAACCAGTG
TCTGAGCCGAGCTTCCACCCATGCTCTTTCAGAGGTGTGGCCCCAGGCACAGTGGCATCCCCAGCCGGTTCAGTGTTCACTGTGGAA
GAACCTGGGTTAGGAGTTGGACTGTGCCATGTTAAGCTGCCTGGGGTACTTGGTTGTCACCCTTAGTCCATAGCCCCTCCCTCCCCT
ACTCCTGTGCTACAGCCATTGTCACATCCTCGGGCCCCTTCACCACAGCCCAGCTAGCCACAGATCTCACTCCCTCCTCGTGCTTCC
ACAGTGTTTGCCAAGAATTTCCGTGTGGCCGCTGCGATGAAGACATCTGTGCTGCTCAGTTGGGAGGTCCCCGACTCTTATAAGTCA
GCTGTGCCCTTCAAGGTGGGTAGGACTTCTGCCAACCTAGACCAGAGCTGCCTCCCATGTCTGCTCTGCATCGCCATTCCGATCCCA
GCTGCTCTTCCCCCTCCCCCTCCAGATCCTGTACAATGGGCAGAGCGTGGAGGTGGATGGGCACTCGATGCGGAAGCTGATTGCAGA
CCTGCAACCCAACACGGAGTACTCCTTCGTCCTGATGAATCGTGGCAGTAGCGCCGGGGGCCTACAGCACCTGGTGTCCATCCGCAC
TGCCCCGGACCTCCTACCCCAGAAGCCACTGCCTGCCTCCGCCTTTATAGAGGATGGCCGCTTCTCCCTCTCCATGCCTCAAGTGCA
GGACCCCTCGCTAGTCAGGTGTACAGAGGAGGCTGGAGGCTGGTGCAGATCGGGAGCGTAGCAAAGATCGATGAAGGTTCTGATGGT
TGCTCTGTCCACACAGGTGGTTCTACATTGTGGTGGTGCCCATTGACCGTGTGGGCGGGAACTTGCTGGCACCAAGATGGAACACAC
CAGAGGAGTTGGAGCTGGACGAGGTGCCTAGGGGAAGATCAGTACTGAGGGCAGCCACGTGGTGGGCAGTGGCCATCTTTCCCTGAG
TGTTGCCCCAGGAGTCCAAATCTGGCTGTCAGCCGCTCTCTCTCTCTCTGGGGAGAGGACCTCTGAGGAGTATCCCCAGGAGAATTT
TTAAAACTCCGGAAGGGCCTACAGGGACTTCCCTGGAGGTATTTGGTAAGAATCCCATGGTATAGGAGTCACTAGAGACATTTCTGC
AGCTCTCAGTGTGGACCTCAGAGATGCCTGTGGTGTAGAGCTTTAAGCCTAAGTTCTCCTGAACAGATGAAATCACCTCTTTCCTCC
TCACAGCTCATGTCAAAACTGCTTTCTCTGATTAGGATAAGGTTATTCATGTGGCCCTTCCTTATAGGCTACAAGGAGGGTCTCGCT
GTGATGTCAGAAGTATCCTGTAATGGCTATGGCCAGGCCAGGCCCATGAGGGTCCCCAGGAAGAGGGAACCTCTATATCTGCCTCAG
TAGCTAATGCCCCAGGGGAGGAGGCTCCCAGGCCTTGTGTGACTCTGTGCTCTCCTCTGCCAGCTTCTGGAGGCCATCGAGCAGGGC
GAGGAGAAACAGCGGAGGCGCCGGCGCCAAGCAGAGCGGCTGAAGCCTTATGTGGCGGCCCAAGTGGATGCGCTCCCTGACACCTTC
ACCCTGGGGGACAAGAAGAGCTACCGCGGCTTCTACAACCGGCCCCTGTCTCCGGATCTGAGTTACCAGTGCTTCGTTCTCGCCTCC
CTCAAGGAACCCATGGACCAGGTTGGCCTGAGCTGGCATGACTTTCAGAACCCCAAGACCCCAGGCTTAATCTAAGACCAGGCCAGC
TTCAGACCTTAGGAGACCCCGGGTCTGCATTATCCTGAGACTCCAGCATCCTAGGACTAGGACACCCCTGAGGACCTATACTGGATA
TCCCATGACACTAGGCCTCAGGGTGTGAAGATCAGACCTAACATGGCTTCAGTAACCCTGCTGACCTGTCACATGTCGGTCTCACTG
GTCTCGGTGCCCTCCCTGGGTTTGATGGATGAGGGTGGGGCTTTCTTGCTGTGCTGTGCTCATAGCCTTTGCTGTTGCTTCCTTCCC
TGGACCCCAGAAGCGCTACGCCTCCAGCCCCTACTCGGACGAGATTGTAGTCCAGGTGACGCCAGCACAGCAGCAGGAGGAGCCCGA
GATGCTGTGGGTGACAGGCCCTGTCCTGGCCGTCATTCTCATCATACTCATTGTCATCGCCATCCTCCTGTTCAAGAGGTGAGTGCC
GCGCCTGGTTCTGGGGGACAGAACCTGCCTCCTGCCTCGCTGGTACCCTCTGGTGCAGTGTTAAGTGCCCAGATGGACGGAAACAGC
GGGTCTGTGTGTGTGTGTGCACTCGGCTGTGCAGGTCCGCCTTTATGAAGAAGGGCTGAGGTGAACTGTGCGTGTCCCGAGGCGTGT
CTGTCTGTGTCTATATGTGTGAGTCTGTCATCTTCAGTTTGAAGGAGCCACAGCTTCCTGGTGTCCTGCTGTAAGCACTTTTCTTCT
GCCACCGAGTGACAGGCTCTGTAACGGCTATCACCAGCCCAGGCCCTCCTCTCCTCCTTAGGCCCACAAGAGGTCCAGGCTGAGTTG
CAGACCTACATCCCCATGAGAATTACACAACAGACATTTACTAAAGACCCATCATAGACGTGGCTGTCAACACTTACCTGTCTTGGG
TAGTTGTCGCACCTGTCCTTGGAGGTGCCCTGCTATCCTGTCAGCTTCCAAGCTGCTTCCAATTATTTTACTTGGTCTTGGTCATTC
CCAATATATATATATTTTTTTCCTCCTAATATTTTTAAGATGAGGTCTCACTGTATAGCCTTGGCTTCCCTACCGTGTAGTCCAGGG
TGGCTTCAGACTCAGAGACCTGCCTGCTTCTGTCTCCCAGGCTACTCTAGAAACTGAGGTAGAAGGATCCAGGCCTAACTTATCTAA
GGCCTCGAACTTAGGAATCTGCCTGCCTCTGCCTCCCAAGTGCTGGGATTAAAGGCATGTGCCACCACTGCCCAGCTTGTTTTGTTT
TTTAAGGTGAGGTTCTTTGGCTCGGTGTGGAGTGCCTGCCTTTGCGTGTACAAAGTTCTGGATCCAATCGCCAGTAGCACAAAAACT
TAAAAAGTTTTTTTAAAAAGCCTAACCTTTGAGCCTGACAGTTGAAATGCCTGGTTAAATGTGTCTTTAGCATTTCCCTTGACATCA
TTACACCAAATAAATACGGTCCCTTGGCCCCTGGCTCCTGCTCTGCAGCAGTGCCCCCCCCACACACACACACACACACAGCCCCAG
TGCCCCCTCACCTGGCAGCTGTTCATACCATGTTCTTACTTCTAGTCTCTGGTGCTTCAGAGACCCCTGGCTCCTGGTCCCATCTTT
ATACAGCCCCTCATCTACCCCAGAGTGCCCCACTGGATATGTGCTGAGCCCCCAGCAGCTAGCACATGGCTAGCTGTTGAACATGTC
CTGTGCTATGTGTGATGTGTCACCTCATGGAGGTACCCACTTCCCTGTCGTTATCCTGCCCTCATCTGGACCCCATAATCCATCCTG
GCAAGTTCCCTCCTTCCTCTTGCGGTCACAGACCTATAGGTGTCACTAAACAGTGTCCCTCCACCCACTTACCCTCACCATCCCCAC
TCAGGTTCTGATGTGTGTACATGGAGGTGAACTCCCTGAGGTTGTCCTATGGGTACCTTATAAGAGTCCCATGGTTCCCTTGTCATG
TGAGGGCTGAGTGCTTAGCAGCAACAATGACCATTCCTTTCTTCTGTCCTTGCCTGTCTCTCGGGACCTAGTAAACAAGAAAGGTAG
GAGTCGTTCCTTCTCTCTGTACACGCCTCCCCCTCTACTTATCCCTGTGCACATCTTGGGCCTGCAGGAAGGGGTGGAGCTCTGACC
AAACCCTCTTAGCCTGTGGCTTTAATCAGCAACCTGCCTGCTCAGCACAGGAGGCTTCTCCAGTTCTGGATGGATCCTCATGGGCAC
GAGGGGGCAGTCTCGATCACATTCAATGTCAGTCCAACTCTGTCCCTTCCGCAGAAGATGCTCCCCACTGGAGCTGTGCACGCTGCA
GCCCCCATTGCTTCCCTGAGTCCACAGAGCTCTCCAGAACCCTCCTTAGGTTCCCATTTCTCCATCCTAATTCCTACGAGAGGCCAC
CTCTGCACTCGGCAGAGCAGCAAGGAAAGGTGTCGTGATGCAGACACGTGCGGGCATATTTCCAAGTTCTCCCCGGACACTCCTGTG
CAGGGGCTGCACGTGGTCCCCAGTAGTCTGTCTGACCCTGTCCTTTTTAGATGGGCAAGGTGACACCAGTGAGAGAGGACTCTGTCT
GGCTAGGGCCCTGCTGTTCCTGCCCCTCCCCTGCTACTCTGGACATTGGCCCTGAGAGCCAGGCAGCTGGGACCCAGGCCCTCGTGA
TGGCGTTTGTGTGTTCTGTGTACCAGTGCATGTTTTCTCGTGGTGGTTTCTGCTGCATCAGCTGGGGTCGAGTTAAGGACAGCAGCC
ACTGCCCAGCGGTGACCTGCCTCCTGTCCAGAGGAACCACCAGCCTCTCTGGCCTCCCTCCCTCGCTGCACTGCTCCTGGGCCCACT
CCACACCTGCCGTCTCCCTGCCCTGTCCCTACCCTGTGTTCTCTCATGTTGACGCTGCTCTCCCCCTGGCTCACACCCAGAGATCCC
AGGATAAGGTGGGGTGGTGGGAAGACATCTGGGGCCGTGGGAGCTGCCTCTGTTCCCAGTTTTCACTAGCCTCTTCTTGTTTCTTTA
GGAAGAGAACACACTCCCCATCATCAAAGGATGAGCAGTCAATCGGGCTGAAGGACTCCCTGTTGGCCCACTCTTCTGACCCTGTGG
AGATGCGAAGGCTTAACTACCAGACCCCAGGTAGGGTTGATCCCTCGGCCTCTTGGCCTCCAGCTCAGTCCCACAGGTTGTCAAAAG
ACCTTAGTGCACGCTCACTTCTGGCTGCTTCTCTTCCTTTTTTGTTCCGTTTGAATTGACATGATGGGACTTCATGATGGATGGGGG
CATCCATATTGAACAGGTGGACAGGCTAGGAGACTTCTAGAGACAACAGAGGTCCCTGTGTGTCTCCAGTCTCTGAATAGATGGACT
GTAAGAGACCTCTTGAAGCCCTCAGCTCTGGCACAAGTGGTCAGTCTCAATATCTGCCCTCTGGCCCTTAACAACAAACACACACAC
ACACACACACCAGGCTGTGTTAGAAGTGAGAAGCTGTAGCAGACAGAATGGTAGGTGAGGGGCTCATACCTGGCCCTTGATGAAGGC
TATTTCAGGTTCACTGTGCAAGGGTGTAGGGTTCCTGCTCCAGGCTCAATACCTGTCCCTTCCCCTGAGCTGCTCTCATGTGACTTC
GAACTGTTCCTTCCACCTGGAATATCACTCCTTCCCTACACCCCAGAGGTGTCTCTCAGAGAGCGACAGTCTCTAGCTGTAGCTCCA
GGACCTGGCTGGCTCAACCCGTCCTTGTGTTTATCGTGTGGTACATTCTCGCTGGATGCTTGAGCACACAACAAAGGAATGAGTGAA
TGAAGTCATGTGTGCACACTGAGGGCTTATGTTTGTGTGAGCTGGTTATCACTGCGCATCAAATTACCCCTAACCTTCAAGGCTTAA
AACAACAGTAATTATGTCTTCTCTCTCACAGTTGTTATGAGTCAGGAATTGGGACAGGGCACGGTAGGATACCCAGCCAGGGTAGGC
CTTCAGCCGGAAGATTTGAAAGCTGGGAACTAGAATCTTATAAAGTGGGGCTGGCAAATTATGGCCTGGAGCCAAATACAGCTCACT
GTCTGCTTTATAAATAAAATTTGCCCAGCACACCACCACACCCATTCATTTCTGGGGACATTTGCACTACAAAGGTCAGTTGACAAC
TAGAAGGTGGAAGAGAACCCTGGGTGTCTGCAAGCCTTTAGCACTGGTTTTCTGGCCTTTGGTAGTGAACTTTGCCAACCCTAGGGT
GGTTTGTTCCGGTGGGAGGTAGTTGGCAGACGAGCTAGCCTCTTTGTGTAGCCTGGACTTCCTTATAGCATGGCGACTGAGTTCCAA
AGACACGTGTCTTGAGAAAGTGTTCCGTGTGGACCCAGCGGGCTGTTGTAACCAGTGCGTGAATCTGTGGTTAGAGGAGGCTGCTCT
CAGAGGGTGCCTCACTTTGTGGGCACCAGACAGAAAGTTGGGTTGGAGGTGGTTCTAAGTGGAAGCACAGTGTGTATACATCAAGTT
AAACCCCTGAAGGGTATTTCTGCAGCATTGGAGCCAAGAAGTGACCGTGACCGGCAGGCTGAGCAGCTTAGACTTCATCCTGACTGA
CCAAGGGAGTCATTGAAGGTCTTCATCAGAGGAGCAGCAGAGCTGGCTGCTTCTGTGGAGAGCGGGCGGGCCCTGGGGAAGACATCA
GGCAGTTAAGTGGTAGGAAAGGGAGGCAGGAGAAGAACAGAGTCTGGGAAGCTGAAGGTGGTAAGGCAGGTGAGCTGAGCTCACACA
AGGATGCAAAGCCAGTTCTGTGTCTGCTGCCATAGAGGATATCCAAGTGGCAGTAGGCCTTTGTGTCTGGACTTTGTGCTGACCTCA
GCCCGGGAGCCACCAGTGTGAATGGTACCAAGGATCAGGTAGTGGTCAAAGCTCAGAATGGGCAGACTCTCCCGGGACGAGCAGGAA
GGGAGAAGGGAACAGGCCGCAGGATGGTGGTGTTATAGGGGAGCATGCAGGCAGAGGTGTGGGCAGAAAGGTTCCCTGTCTCAGGTG
CTGGCGTAGTGTGGCACTGAGAGGGTAGGGCCCATAGCTGTGTTGAGGTATAGGACCCAAGCCTTCAGGCAGAAGTATTTCCATGGG
TTGAAAAGCAGCAAAAGGGAATGCAGAACGCTTTTCCATATCAGTGTGTGAAGGATGTCATGGGCACATGACATCCCTGAGAAGGGA
GGAACTTGAGATTTTAAAAGATTTTTCATTTCCGGTATGGAGACCATCTGAAAATGGCCAAGGTCAGATTTTGAGGGGGTGAGGGCT
GGGCTGGAGTGACTTTATTATAGGGTCTCAAGAGGCCACTCACACCTATGTTGCTTGGAAGCTCTCTCTTTGGGGGAGGGGGACTGT
CTGTTACTGGAAGGCACGTGAGCTGACCCACTCTTTCTGTCTGTGCCCCTTCCTCTTGCCCTTCACCTACAGTCCCTTCAGTGTCGA
ACCGCAGAACCAGGTGACCAGAAAGAGGGTTCCTGAGCTCTGCTCCTGTCCCATTGTTTTCTTTTGGTTTGTTTTCTCTGCAGGTTC
CAGTGCCCCCAGTTGCCCGAATATCTCAAGTTAGTTTTCAAAGTTCCCACATTGGTTTGGGGACACTTTGGCTTCAGTGTGTCTCCA
CTCTTCTACTTCCTCACGTCTCCCCACTTTCTCCTTGCCCTTTTCCCCTTGCCTTTATCCCTTCCCTCCCCTTCCCGCGTCTCCCTT
CTCTCTGTCCCCTCCTGTGCTCACTGGCTTCCTCCTGTATCTGCTCTCCCACCCTCTCTCCTTGTCCCTGCCGCCTCTGGTCTCCAT
CCAACCCCTCTGTGTGCCTCCGTCCTGATCCCTTGCCTGTCTTTGTATCTTCCTCCCCATCTCTGCTTCTCTCTCTCCCTACTTCTA
TGCCACTCTGCCATTGTCTTTGCGCTCCATGACCATTCCACCCACACACTTGGTGCCCGGGGTGACAGGGTCACCAAATCTCTGAGG
TGACTGGCGTCAATGAGGGGTCGGGGTAAGGGGGTGTCTGTCTCAGACAGGTGTGGTAAGAGCCCAGCTTCCACCCTCTTCCTGAGC
TCGAAGCCCCGTCTTAGTGGGAAGAAGCCCCTTGCTCCTCTTAGGCAGGCCTGGCAGGCAGAGAGCCCTGTTGTCCCGTGCAGCACC
AGGTCATCTGGACATGCTTCTGTGTGCTCACAGTACACACACCTCCATCTCTGGCCTCCCCTGATGCCCAGCCACTGTGTGTTCTCC
ATTCCTGTTGCCGCCGCCGCATGTGCTGTTGTCTCCATGCCACCAGTGCTGAGTACTTCATGATCACACATGTTCACACATGCCTTG
GGCTGTCTCTACCACCCTGACCAGAACTCACACTAATGCCACCCATGTGTCTGCCCCACCTCCCCCAGGTATGCGAGACCACCCGCC
CATCCCCATCACTGACCTGGCAGACAATATTGAGCGCCTCAAAGCCAACGATGGGCTCAAGTTCTCCCAGGAGTATGAGGTGAGACA
CCCACCACTCCCACCGTGTGTCCTCAGTCTCTCCAAGTCTGGTTTTGTTTTGTCCTGGGTCCCACCTGAGGTCGAGGATATTGGGAT
GTAGGCCAGGGAGATCAAAAGTTCAGTTGGGGAACTGGCCGTCATAGGTGTTGGGCTCAGCCCACAGGCTCACCATAGCCCTCTGGT
GGTCACCCTAGGACATGCTTCAGAAGGCTCTGCTAGGTGATGCCTGCAGAAGCAGACACTGATTTAGTAGCAATGGCTATAATATCT
AATCTCCAACAAGAACAGGCCTGCACTTACCAGGTACGGGACAGCATCCTCTTACTCGGTCCCCCGTGGAGTGGTACTGTCACAGTC
TTGTCTTACATACCAGAAACCAGACTCAAACAGAGGTGTGAGTTACCTAGAGTCAAACAGCTGAGGAGTCGTGGAGCCACCCAGACC
TTGCCTGTGCTGGTTTCATGACCATCCGCACTTCAGTCTTGCATAACAATGTTACTGAGTCTCCACATGGTAACCATTGTCCGAGAA
GGGTGGGAGGGTCTGTTCTCAGCCCCGCTTCCATTCCCGCTCCCACATGTGATCACACATGCATGCCGAAACACATAGCTCATAGAT
GGGGAGTCAGGAAGCCCTCCAAGTGTTCGCCTCACTCTGGCCTTCACCTCTGTTTGCTAGCTCAGACTTCAGGGTCAGTGACCACCT
GGTTCACCCACAGCTCTTTGTCAGGGACCTTTGGGTGTTCCCAGGCCATACAGTCAGGGGAGCCTTGTTAGAAAGGAAGTGCTAAGG
AGACCCCCTTTCTTTGTCTCCCTGCTGTACTGGCCGCTCTTTTGTCAGCACTTGGCATACACCTCAGAGGATGACTGATGGACAGAT
GATGGATAGATCTGTTCAGCTGGACCCAGGGGGTTGCTCTCTTTTAGTCAGGTGTCCACTCTTGAGTTATGTTTGGGGGATATATCC
CTGGGTACCGGATTTGCCCTTCCGAGGGCACTGGGCCAGGTAGATCCTAAAGGCGACAGGCCAGGCTTCCAAAGAAGAAAACTGAGC
TTGGCAGGTAGACAGATGAGCATGCCTCTGCACAGCAACAGTGTCACCTCTGCTGTGGAGGCAGAGCCTGTCAGCCGGGTGGCCTGG
ATTTAGCTTAACTTCTGGACAAGGAGGCCTTCCTTGTTTTTTACCAGTATTCGCCCTTGTCCTGCCCCACGCCTTGGCATCCTTCCT
ATAAGCCCCTGCTCACGGCCGTCACCTTACCACCTCCCCTCCATCCCTCCCATCTGAGCGTTCTTTAAAGGCCCTTCTTTGTCCTAG
AAGCCACCAGATCTATTGTGGGCTTTCCCGAAACTTTCTCTTCATGGTCTGTAGGTCATTTGTCAGACTGTTGTTAGCACTTAACTG
TATTCCAGTCCCTATGCTGACATGTAGAAGCAGATGGCCTTGGGACGCAGTCACTGAGAAACACAGACAGAGCAGTAATCGTTGCTG
TGAGTGTGTGGTTCTGACAGCATGCACTAAAATCATATATAGCAAACACAATGGTGGGTGTGTCTGGTGGGTGGGGCCTCACAGAAG
TGATGAGACAGCCTCCTTAGAGAATAAGGTGGCTGGGACCAAAACGGAGGAGACTGTGATACAGGCATCGATTCTTTCCATCCTCAA
AGGAAGGACAATAACTCAAACCTCTCTTGAGTAAAAAGATGGAGAAATGCACGCAGTGGCTTAGGACCGTGCTGGGCACACCGTGAG
CTCACACAGAAATGTAGCTGTTAGCATTTTTGTGTCCTGAGCCAGTCAAGAGCATGGGAGGCCTCTGAGCAGGAATTTGATACTCAG
ATCTGCATTTTTTTTTTTTTTGAAAGACTGTTCTTGCTGAAGGACAGAGAGGGTTGGAGCAGGGCAAGAGAAGAGGCCTGGAGGTAC
CAAGCAGAGCTAGCTGCTGGCAGCAGCAGCAGGTTGAGAAAGGCAGAGGAAAGGAGTGTACGGGGGTGTGGTAAGCAATGGGAGGGA
CCTTGGGGAGGTCCCAAGATGGACTTTAAACCTGGCTGGGCTTCCTGGGTCAGTGATTTGTGCTATCCACTGAGGGGTGGGGGGGTC
ACATCAGAACCTGCTTTGACAGGAAACAGATGAGCTGGCCTTTAAAGGGACTGGGGTGGAAAGAGCTGTGAAGCTTGCACATGGAGA
TGTCTAGGGGGCCGCAGGATAGTAGGGGCAGCTCTGAGATGTAAGCAGGAGAGTCCTGTGAATGGGGTGGCGGTGGCTAATGGGAGT
GGTTCAGTCAGTGAAGATTGACTTACAAAAGGTGAAGATTGCAGCTATAGCTGTGTAAAATGGCCACTTGTAATCCAGAGTGGGGAG
ACAGAGACAGAAGCCACCATGGTACTACTGGCTAGCCAGCCTGGCTTACTTGGTGAACCGGGGGCCACTGAGAAGTCCTGGCTCAAA
GGCAGAGGGTGTTGCTAGGGTTGTCTACCACACAAGTGCACAAACACATATGTGTACACACATGCATTTTAAGAGAGAGAGAGAAAA
GAAAAACAAATCTTGTGAATGGGCTATGGATGAGACTAATTTGGTAGAGTGCTTGCCTAGAATTTGCAAAGCCCTGGATTTCCTCCA
GCATCACATAAATGGGGTAGCACACACTTTAATCCCACCACTCACCCACACATAACATGGAAGTAGGAAGATGAGAGAGAACTTTGG
AGCCATCCTTAATTGTATCCTATATAGTAGGTAGTAAATTCAAACCTAGCCAGGTATACATGAGACCCTATCCAAACAAGCAAACAA
ACAAAACATTTCCTGAGCAACGAAGTGAGAAGAGAGCCAGGGAAGAGATGTAGAGGAGCTTAGATCCTGCCTACGGGGAGGGCAGGG
AACAGACAGAATAGGAAAAGCCAGAGATGGAGAAGAGAGCTGGGCAGCATGGTATGTGGACCTGTAGCCTTGGACAGTCAGTCTCCA
GCACAGCCCAGGTGGCCGCATCATCATCATCATCATCATTATCTCCATCTCATTCCACTGGTTGGATGCAGGGCCTGACCCATGACC
CACCTGGTCTTCATAAGCTCTACAAGGGGCTGCCATGGAAGTTTGAGTCTTCAATTTATCTCTTTCCAGACTTGGGCTGGGGAATGT
AGGTGTTACTTGGCTGCAAAGCACCATGGATAGGCCTCCCACGACTTGGCCCTCACTTTCCCTGTTTCTTCCATTTCTGGCAGTCCA
TTGACCCTGGACAGCAGTTCACATGGGAGAATTCCAACTCGGAGGTGAACAAGCCCAAGAACCGCTATGCAAATGTCATTGCCTATG
ACCATTCTCGAGTCCTCCTCACCTCCATTGATGGTGAGCCCGGGGCACAGTGCCCCCTTGCCCTTTGCTGTCCTGTACCCTGCTCTC
CAGAATGCTCTGAGCCCCAGGTTAACACTCTCGAATCCCTTTGCATTCCTGTAGGTCTTCCTGGGAGTGACTACATCAATGCCAACT
ACATTGATGGCTACCGAAAGCAGAATGCCTACATCGCCACACAGGGTCCGCTGCCCGAGACCATGGGCGATTTCTGGAGGATGGTGT
GGGAACAGCGCACAGCCACAGTGGTCATGATGACCAGGCTAGAGGAGAAATCCCGGGTGAGGGGGGTGATGCTGGGTACCACCTCCT
GTGAGTCTTTTCAAGGCACTCCCCAAAGGCTGTGGTTTTCCTGGAGTATTGTCTCATCCCCTAGATAGAGGGGAACTGCTGGGATGT
AATGTAATGTGGTCCCCAGGTGTTTATGGGTTCCCGGGATGTTAATATGTTTTACCAAATGCTGTCGTTTTCTGGAATGTTAATATG
TTCCCCAGTTTCTGGGGTGTTCCTGGGGTATTCACATGTTCCCCAGATGCCAGAGTGCTCCTGGGGTGTTCACACATCTAAGTTAAT
TAGTGGAAGGAGCTGCTGATGTCTGCTTTTGCACCGCGAAGCATTGACTGGAACATAAAATTTACAGAGCTTGAAAATGGGGCATAA
ACGTTATGGGTAAATTTGGCAGAAAATGTGCCTGGTACGATCTGGTGTTGAATAAATAATTTTCAATTATGGTCCTCCTTCAGCCAG
TGAGGGCCAGGGTGGTAGCTTAGTGTGAGGAGGTTTGGAGAGGAAGAGGGAAGTGAGAATCGAGGGAGCTGGGGCCTTCCCCTTGCG
TCTGCCTGTTTGAGCCTTTGGGCATCCTATAACTTTGTCCCCTGTCCTGCCCATCCCCCTCCAGGTGAAGTGTGATCAGTATTGGCC
AGTCCGTGGCACTGAGACCTATGGCCTCATTCAGGTGACCCTGGTGGACACTGTGGAGTTGGCCACATACACCATGCGCACCTTTGC
CCTCCATAAGGTATGTCCCTGAGTTCCCAAGCACTGGCCCATCCCTTAGGTTGTAGCCTGAGTGTGATGGGCTGGCGGAGGGCAGCT
GTGGGGTCAGGGCCTGGGAGGTGTATGAGGGGTTCTCACGGCTTGTGTTAGGTGACTGTCTGCTGTGGCTTCTGAGTGTCACCTGAG
AGTGGCTGGTCTGGCCCCTTCCCTGGGTGTGTGGCAAGCAAACCTCAAACCTCATCTCCGTTGACCTTTCCCAGAGTGGCTCCAGTG
AGAAGCGTGAGCTGCGTCAGTTCCAGTTCATGGCCTGGCCAGACCACGGCGTTCCTGAGTACCCCACTCCCATCTTGGCCTTCCTGA
GACGGGTCAAGCCCTGTAACCCACTAGATGCGGGGCCCATGGTGGTGCATTGCAGGTGAGAGTCCAGAGCCACCAGAGGGGAGGGGT
GGGAGGTGAGGGGACCCCTGCCCCAAGCTCTGCTCTTCTCCTCAGTGCGGGTGTGGGGCGCACAGGCTGCTTCATCGTCATCGACGC
AATGCTGGAGCGTATGAAGCACGAGAAGACGGTTGACATCTATGGCCACGTGACGTGCATGCGCTCACAAAGGAACTACATGGTGCA
GACCGAGGACCAGTATGTGTTCATCCACGAGGCCCTGCTAGAGGCTGCCATGTGCGGACACACCGAGGTGCTCGCTCGGAACCTCTA
TGCCCACATCCAGAAGCTAGGCCAAGTGCCTCCCGGGGAGAGCGTCACGGCCATGGAACTTGAGTTCAAGGTGGGACTCCAGAGGCC
TGACTTGGTCGGTCGTGGGAAGCCTGTTTGCAGCAGGCCAGGCCTGACAGAACCCTTGCTCTTCACAGTTGCTGGCCAACTCCAAGG
CCCACACGTCGCGCTTTGTCAGTGCCAACCTGCCCTGCAACAAGTTCAAGAACCGCCTAGTGAACATCATGCCCTATGAGCTGACCC
GAGTGTGCTTGCAACCCATCCGTGGTGTGGAGGGCTCAGACTACATCAATGCCAGCTTTCTAGATGGCTACAGGTCGGCATGCTTCT
CCCTGCTGCCCCACTGTGCCCCTTCTGGTACCTTGGTCTGCTTCTGGTTGGGAGAGTTAAGAGCTAATCCAAAAGATCAGAGTGAGT
TGGGGACAGAAGTGAGGGCTTCCTGGAGGAGGGCTGCTCTCTGAGGAACTAGACAATGTCAGGATAGGTGCGGGCAGCAGCACCTCC
ATCATGTGAAGTCTTATGTCACCCAGACAGCAGAAGGCCTACATAGCTACACAGGGGCCTCTGGCAGAGAGCACCGAGGACTTCTGG
CGCATGTTATGGGAGCACAATTCCACCATCATCGTCATGCTGACCAAGCTTCGGGAGATGGGCAGGGTAAGCCCACCCCTTCTCCTG
GGGCCCCTCTCATACCAGGGGGGACACCAGCTACCCTTGGGAAAGGGGAACCTCCTCCACCCTCTGCTCTCATGCCCCAGCTCCCCA
TCAGCATGGCCTCAGACCTCTTTACCAACACCTGAGGCACCCCTTCCCAAAGTCCTCTGAAAGGTGGCATTCTCTTCTGGCCTCCCC
ACCTGATCTTGGGCGGTGCCTGGTGTTCTGTCCACAGGAGAAATGTCACCAGTACTGGCCAGCAGAGCGCTCCGCTCGCTATCAGTA
CTTCGTTGTTGACCCGATGGCTGAGTACAACATGCCCCAGTATATTCTGCGTGAATTCAAAGTCACAGACGCCCGGGTGAGTGTCAG
CCAAAGAATATGCACACAGATGTGTCCCTCGAGCTACCCGGGCACACGTGGCTCCATCTGCCCCAAGACACTGCAGGGATCTGAGTC
GTGGGCGAGTTGTTTGCGTTACCTCCTAGAGTGTCCTTGAGAGTGCGTCCCTGGGTGGTTGTGCTCCTGGTTTAGAGGGCCTAGTAC
CTAACATCCAAACCTTCATTGTGACCCCTGACCCTCTCTCCCAGGATGGGCAGTCAAGGACAATCCGACAGTTCCAGTTTACAGACT
GGCCAGAGCAAGGAGTACCCAAAACAGGTGAAGGCTTCATCGACTTCATCGGGCAGGTGCACAAGACAAAGGAGCAGTTTGGCCAGG
ATGGGCCCATCACGGTGCACTGCAGGTTGGGACGGCCCCCGTGGAGGGATATGGGAGGGGGGACGGTGGTCCCGAAGGCCAGGGAAG
TTGATTGTGTGAAGGGAGCAGGACTCCTGACACAGCCATTCTCTGAGCAGTGCTGGTGTGGGCCGCACCGGTGTGTTCATCACCCTG
AGCATTGTCCTGGAGCGCATGCGGTATGAGGGTGTGGTTGACATGTTCCAGACCGTGAAGACCCTCCGCACACAGCGCCCTGCAATG
GTGCAGACAGAGGTATGCAGGCCAGAGGGAGGGACCCACGTGTCTTTGCTACAACCCACAGCTGCACCTGCAGCTGGCCCACGTGTA
TTTGTTCTCCTTATAGCTCTGTGGTATTGCTTGTCCCACATGGCGTGCAGGGAAGAGGAGAGGGAGCTGTGTTCATTCCGTAGCCCC
TTCCTGAGCGCCAGTGTGTCATGTTGCCTTTGGGGTCAGAAGTAGACGTAGCAGTCTGTGTAAGACGGTGGGTGAGTCTGGGTACAG
ACAAGTGGTTTGAGTAGCAGAGGTGAACACAGGCCTGGGAGAGGCAAAAAGGAGAAATGGCCTTTCGAGACTTCAATTGGAGAGTAC
CTGGGTAGTTCTCGGTGTTCAGCATCTGCTCTTAGCCAACAGAGGCCATTTCTGTGGCCACTGGCTGCAGCACATGCAAACCCTTGT
GTTTGGAATAGCTATTGGCTCTGTTCACACTGACCAGTCCCCCTCTTTCCTCAAAGGACCAATACCAGCTGTGCTACCGTGCGGCCC
TGGAATACCTCGGCAGCTTTGATCACTATGCAACGTAACTACTGCTCCCCTCTCCTCCGACGCTCCCCCGCGGGGCTCCGGAGGGGA
CCCAGCTCCTCTGAGCCATACCAACCATCGTCCAGCCCTCCTGCACGGATGCTGTTGCCGGCAGAGCACAGCCCACTGGGATCACAG
CATTTCGGGGAACATTGCCACACCAGTCAGAGAGCCCAGAACACCTGGGCAAGTAGGCGGACTGGCAGCCTGGCTCTGGGCCCTCGT
CCACCGGGCCAAGTGGAGCCCCGCTTCAAGCTCTCTGTTCAGCTCCGCGTTCTCATGCTTCTCATGGGGTGGGAAAAGGGGGCAAAC
CCCCCACTTTTTATACACTAGGCGGGGTAGACTGCGGGGTCCTAGCCTCTTCCTCCGACTTTGCTTTTGCAGGTCTTTCACTGCAGA
TGGGGCTGCTGTGGGAGTTGGGACTTGTTTGTTTTCCTTTTTGAGTTCACGTTGGATCCTTTCTTGTACAACTTCTGCGGAAGGACA
CAGTAGTAACTCGCCTTCCTTGTGCAGAGCTAGGGCCCTACCTGAGCAAGTCGGCTGTGGCCCGGGAGGCAGCGTGACTCCTGCTGT
CCTCCAGCCTTTCAGATGAGATCCTATTCCAGCCAAATGCAGGGAAACACTTTATTTTGTTTGTTTTAGGTTTTGTTTTTCCTTGAG
AGCCTTTTTTTAGGCCCCACAGACAGTGGTGGCTGGGGAGGCGATAGGAAACACATTCCCCAGTGTGCATTTAACATTCATAGCCTA
CAACCACAGACGTGTCTGGGGGAGCCTGGCAAGGCGTTCCTCGTCACCATCGTGTTTGCAAAGGTTCAAAAAACAAAAATCAAAAAA
AAAAAAAAACATAAAAATATTTTTTTTTAAGAAAAGAAATAAAGATTCATCCCCTGGCCTCTACTTCAGATTCGAAGTGGGAGGCAA
CTCAATGTGCCTGGGGCTCGCCATGGGCCCACAGAGTCTCGCTATCATCCCCAGCGTCGCAGTGTGGCAGGTGTTTGTAGGCTGTGG
GTTCTGGCCACCTCGGGAAATGAATGGCACAGGTGAOGGCCTGTGCCACGCCCCACACACCTCAGGGCCAAGCGGGGGCGTGGCTGG
CCCTTCAGGTCAGGCCAGTGGGCCTGGTAGCACATGTCTGTCCTCAGCCGGACAGATGCTTTCTCTCCTGGTTTGCAGCTGTCTTCA
AATCCCCCCATGACCCCCTCTTCCCACTCCTTCAAGTTGCCCTCACAAACCAATGTGGCAAGACTACTGGACTTGTATCCATGGTAC
TATACTCAGTCCTCTTATCTCAGCTTGCTGAGGGGCAGGGAGAGGGTCTCTTCCTCTGGGCAGCACTATCTAGATAGGTAAGTGGGG
CCGGOGAAGGGTGCATAGCTGTTTTAGCCGAGGGACTCGATACCGACGTCCCCAGATATAGCTAGGCTAAGTCAAGATCAACAGTCC
GGGGTTGGTGGTGTTGGATGAAACATTCATTTTTACCTTGTGGATGCTAGTGCTGTAGAGTTCACTGTTGTACACAGTCTGTTTTCT
ATTTGTTAAGAAAAACTACAGCATCATTGCATACTTCTTGATGGTAATAAATTTGAATAATCAAGATTTCTTACACACTAGGCCTCT
GTCTCAGCTCCTGTATCTAGAGTGTGGTATCTTAAGTCTATGGCTGGGGTGGGTTCTGAGGAGTC
MOUSE MRNA SEQUENCE: mR28-021.1 (Seq ID No: 64)
AGGAAGGAGCGGAGGCCCTGGTGCCCTGCCCTCAGTGCTGACTGCCCAGCGAGAGTGACTGGTATAAGCAAGCAGAGACCCGGTGGA
TTGTCTTAGGCTGTGGCTGGCTGTGGAGCTAGAACCCTGGATGGCTCCCGAGCCAGCCCCAGGGAGGAGGATGGTGCCCCTTGTGCC
TGCTCTCGTGATGCTTGGTTTGATGGCAGGTGCTCATGGCGACAGCAAACCCGTCTTTGTTAAGGTCCCCGAOGACCAGACTGGGCT
CTCGGGAGGGGTGGCCTCCTTCGTGTGCCAAGCCACAGGGGAACCCAAGCCTCGAATCACGTGGATGAAGAAGGGGAAGAAAGTCAG
CTCCCAGCGCTTTGAGGTAATTGAGTTTGACGATGGAGCAGGGTCAGTGCTGCGGATCCAGCCATTACGAGTGCAGCGAGACGAAGC
CATCTATGAGTGCACAGCCACGAACAGTCTCGGGGAGATCAACACAAGTGCCAAGCTGTCAGTGCTTGAAGAGGACCAGCTGCCGTC
TGGGTTCCCGACTATCGACATGGGACCTCAGCTGAAGGTGGTGCAGAAGGGTCGCACTGCCACCATGCTGTGTGCAGCCGGTGGGAA
CCCAGACCCTGAGATCTCTTGGTTCAAAGACTTCCTTCCTGTGGACCCTGCTGCAAGCAACGGTCGTATCAAACAGCTGCGATCAGG
TGCATTGCAGATAGAGAGCAGCGAGGAGTCTGACCAAGGCAAGTACGAGTGTGTGGCCACCAACTCTGCAGGCACACGCTACTCGGC
CCCCGCCAACCTGTATGTGCGAGTGCGTCGCOTGGCTCCTCGTTTCTCCATCCCTCCCAGCAACCAAGAGGTGATGCCGGGCGGCAG
CGTGAATCTCACATGTGTGGCAGTGGGCGCGCCCATGCCGTATGTGAAATGGATGATGGGCGCCGAGGAACTGACCAAAGAGGATGA
GATGCCAGTTGGCCGAAATGTTCTGGAGCTCAGCAATGTCATGCGATCTGCCAACTATACCTGTGTGGCCATCTCTTCATTAGGCAT
GATAGAAGCCACGGCCCAGGTCACAGTAAAAGCTCTGCCAAAGCCTCCAATTGATCTTGTGGTGACAGAGACAACCGCCACCAGTGT
TACTCTGACATGGGACTCTGGAAATACCGAGCCTGTGTCCTTCTACGGCATCCAGTACCGCGCAGCTGGCACAGACGGCCCCTTCCA
GGAAGTGGATGGTGTGGCCAGCACCCGATACAGCATTGGTGGCCTCAGCCCCTTTTCGGAATATGCTTTCCGTGTGTTGGCTGTTAA
CAGCATTGGGCGAGGACCACCCAGTGAGGCAGTGCGAGCACGCACGGGGGAGCAGGCACCCTCCAGCCCTCCGCGCCGTGTGCAGGC
TCGAATGCTTAGCGCCAGCACCATGCTGGTCCAGTGGGAACCACCTGAAGAGCCCAATGGCCTGGTACGGGGATATCGCGTCTACTA
TACCCCAGATTCCCGACGCCCTTTGAGCGCCTGGCACAAGCATAACACTGACGCAGGACTCCTTACCACCGTGGGCAGCCTGCTACC
TGGCATCACCTACAGCCTCCGAGTCCTTGCCTTCACTGCTGTGGGTGACGGCCCACCCAGCCCCACCATCCAAGTCAAGACACAGCA
GGGAGTGCCTGCACAGCCTGCGGACTTCCAGGCTAACGCTGAGTCGGACACCAGGATCCAGCTCTCGTGGCTGCTGCCCCCGCAGGA
GCGGATCGTCAAGTATGAGCTGGTGTACTGGGCAGCCGAGGATGAGGGCCAGCAGCCCCATCAGCCCCTCCCCAGAAGGTGACATGT
GTGAGCACGGGCTCCACCACGGTCCGGGTAAGTTGGGTTCCACCGCCGGCCGACAGCCGCAACGGCATTATCACCCAGTACTCCGTG
GCCTATGAGGCAGTGGACGGCGAAGACCGTAAGCGACATGTGGTGGATGGCATCAGCCGTGAGCATTCCAGCTGGGACCTGCTGGGC
CTGGAGAAGTGGACGGAGTACCGGGTGTGGGTGCGGGCACACACAGATGTGGGCCCTGGCCCTGAGAGCAGCCCGGTGCTGGTGCGC
ACCGATGAGGACGTGCCTAGCGGGCCACCACGGAAGGTAGAGGTTGAGCCTCTGAACTCCACTGCTGTGCATGTCTCCTGGAAGCTG
CCCGTCCCCAACAAGCAGCACGGACAGATTCGTGGCTACCAGGTCACCTATGTGCGGTTGGAGAATGGTGAGCCCCGAGGCCAACCC
ATCATCCAGGATGTCATGCTGGCTGAGGCTCAGGAGACCACCATCAGCGGCCTCACCCCCGAGACCACCTACTCCATCACCGTCGCT
GCCTACACCACTAGGGGGATGGTGCCCGAAGCAAGCCCAAGGTGGTTACTACAACAGGGGCAGGACGGCCCACCAATGATGGTCAGC
ACCACGGCCATGCACACCGCGCTGCTGCAGTGGCACCCACCCAAGGAGCTGCCTGGAGAGCTGCTGGGCTACCGTCTCCAATACCGG
CGGGCCGACGAGGCGCGGCCCAACACCATAGACTTTGGCAAGGATGACCAACATTTTACAGTCACCGGCCTGCACAAAGGGGCCACC
TACGTCTTCCGGCTCGCTGCCAAGAACCGGGCTGGCCCAGGAGAAGAGTTTGAGAAAGAGATCACCACCCCCGAGGATGTGCCCAGT
GGCTTTCCTCAAAATCTTCGAGTGACAGGACTGACCACATCTACCACGGAACTGACCTGGGACCCGCCGGTGCTGGCGGAGAGGAAT
GGTCACATCACCAACTATACTGTGGTCTACCGTGACATCAACAGCCAGCTTGAACTACAGAATGTCACCAACGACACACACCTTACA
CTCTTGGGCCTTAAACCGGACACCACTTATGACATCAAGGTCCGTGCACATACCAGCAAAGGCGCCGGCCCTCTCAGCCCCAGCATC
CAGTCCCGGACCATGCCCGTGGAGCAAGTGTTTGCCAAGAATTTCCGTGTGGCCGCTGCGATGAAGACATCTGTGCTGCTCAGTTGG
GAGGTCCCCGACTCTTATAAGTCAGCTGTGCCCTTCAAGATCCTGTACAATGGGCAGAGCGTGGAGGTGGATGGGCACTCGATGCGG
AAGCTGATTGCAGACCTGCAACCCAACACGGAGTACTCCTTCGTCCTGATGAATCGTGGCAGTAGCGCCGGGGGCCTACAGCACCTG
GTGTCCATCCGCACTGCCCCGGACCTCCTACCCCAGAAGCCACTGCCTGCCTCCGCCTTTATAGAGGATGGCCGCTTCTCCCTCTCC
ATGCCTCAAGTGCAGGACCCCTCGCTAGTCAGGTGGTTCTACATTGTGGTGGTGCCCATTGACCGTGTGGGCGGGAACTTGCTGGCA
CCAAGATGGAACACACCAGAGGAGTTGGAGCTGGACGAGCTTCTGGAGGCCATCGAGCAGGGCGAGGAGAAACAGCGGAGGCGCCGG
CGCCAAGCAGAGCGGCTGAAGCCTTATGTGGCGGCCCAAGTGGATGCGCTCCCTGACACCTTCACCCTGGGGGACAAGAAGAGCTAC
CGCGGCTTCTACAACCGGCCCCTGTCTCCGGATCTGAGTTACCAGTGCTTCGTTCTCGCCTCCCTCAAGGAACCCATGGACCAGAAG
CGCTACGCCTCCAGCCCCTACTCGGACGAGATTGTAGTCCAGGTGACGCCAGCACAGCAGCAGGAGGAGCCCGAGATGCTGTGGGTG
ACAGGCCCTGTCCTGGCGGTCATTCTCATCATACTCATTGTCATCGCCATCCTCCTGTTCAAGAGGAAGAGAACACACTCCCCATCA
TCAAAGGATGAGCAGTCAATCGGGCTGAAGGACTCCCTGTTGGCCCACTCTTCTGACCCTGTGGAGATGCGAAGGCTTAACTACCAG
ACCCCAGGTATGCGAGACCACCCGCCCATCCCCATCACTGACCTGGCAGACAATATTGAGCGCCTCAAAGCCAACGATGGGCTCAAG
TTCTCCCAGGAGTATGAGTCCATTGACCCTGGACAGCAGTTCACATGGGAGAATTCCAACTCGGAGGTGAACAAGCCCAAGAACCGC
TATGCAAATGTCATTGCCTATGACCATTCTCGAGTCCTCCTCACCTCCATTGATGGTGTTCCTGGGAGTGACTACATCAATGCCAAC
TACATTGATGGCTACCGAAAGCAGAATGCCTACATCGCCACACAGGGTCCGCTGCCCGAGACCATGGGCGATTTCTGGAGGATGGTG
TGGGAACAGCGCACAGCCACAGTGGTCATGATGACCAGGCTAGAGGAGAAATCCCGGGTCAAGTGTGATCAGTATTGGCCAGTCCGT
GGCACTGAGACCTATGGCCTCATTCAGGTGACCCTGGTGGACACTGTGGAGTTGGCCACATACACCATGCGCACCTTTGCCCTCCAT
AAGAGTGGCTCCAGTGAGAAGCGTGAGCTGCGTCAGTTCCAGTTCATGGCCTGGCCAGACCACGGCGTTCCTGAGTACCCCACTCCC
ATCTTGGCCTTCCTGAGACGGGTCAAGGCCTGTAACCCACTAGATGCGGGGCCCATGGTGGTGCATTGCAGTGCGGGTGTGGGGCGC
ACAGGCTGCTTCATCGTCATCGACGCAATGCTGGAGCGTATGAAGCACGAGAAGACGGTTGACATCTATGGCCACGTGACGTGCATG
CGCTCACAAAGGAACTACATGGTGCAGACCGAGGACCAGTATGTGTTCATCCACGAGGCCCTGCTAGAGGCTGCCATGTGCGGACAC
ACCGAGGTGCTCGCTCGGAACCTCTATGCCCACATCCAGAAGCTAGGCCAAGTGCCTCCCGGGGAGAGCGTCACGGCCATGGAACTT
GAGTTCAAGTTGCTGGCCAACTCCAAGGCCCACACGTCGCGCTTTGTCAGTGCCAACCTGCCCTGCAACAAGTTCAAGAACCGCCTA
GTGAACATCATGCCCTATGAGCTGACCCGAGTGTGCTTGCAACCCATCCGTGGTGTGGAGGGCTCAGACTACATCAATGCCAGCTTT
CTAGATGGCTACAGACAGCAGAAGGCCTACATAGCTACACAGGGGCCTCTGGCAGAGAGCACCGAGGACTTCTGGCGCATGTTATGG
GAGCACAATTCCACCATCATCGTCATGCTGACCAAGCTTCGGGAGATGGGCAGGGAGAAATGTCACCAGTACTGGCCAGCAGAGCGC
TCCGCTCGCTATCAGTACTTCGTTGTTGACCCGATGGCTGAGTACAACATGCCCCAGTATATTCTGCGTGAATTCAAAGTCACAGAC
GCCCCGGATGGGCAGTCAAGGACAATCCGACAGTTCCAGTTTACAGACTGGCCAGAGCAAGGAGTACCCAAAACAGGTGAAGGCTTC
ATCGACTTCATCGGGCAGGTGCACAAGACAAAGGAGCAGTTTGGCCAGGATGGGCCCATCACGGTGCACTGCAGTGCTGGTGTGGGC
CGCACCGGTGTGTTCATCACCCTGAGCATTGTCCTGGAGCGCATGCGGTATGAGGGTGTGGTTGACATGTTCCAGACCGTGAAGACC
CTCCGCACACAGCGCCCTGCAATGGTGCAGACAGAGGACCAATACCAGCTGTGCTACCGTGCGGCCCTGGAATACCTCGGCAGCTTT
GATCACTATGCAACGTAACTACTGCTCCCCTCTCCTCCGACGCTCCCCCGCGGGGCTCCGGAGGGGACCCAGCTCCTCTGAGCCATA
CCAACCATCGTCCAGCCCTCCTGCACGGATGCTGTTGCCGGCAGAGCACAGCCCACTGGGATCACAGCATTTCGGGGAACATTGCCA
CACCAGTCAGAGAGCCCAGAACACCTGGGCAAGTAGGCGGACTGGCAGCCTGGCTCTGGGCCCTCGTCCACCGGGCCAAGTGGAGCC
CCGCTTCAAGCTCTCTGTTCAGCTCCGCGTTCTCATGCTTCTCATGGGGTGGGAAAAGGGGGCAAAGCCCCCACTTTTTATACACTA
GGCGGGGTAGACTGCGGGGTCCTAGCCTCTTCCTCCGACTTTGCTTTTGCAGGTCTTTCACTGCAGATGGGGCTGCTGTGGGAGTTG
GGACTTGTTTGTTTTCCTTTTTGAGTTCACGTTGGATCCTTTCTTGTACAACTTCTGCGGAAGGACACAGTAGTAACTCGCCTTCCT
TGTGCAGAGCTAGGGCCCTACCTGAGCAAGTCGGCTGTGCCCCGGGAGGCAGCGTGACTCCTGCTGTCCTCCAGCCTTTCAGATGAG
ATCCTATTCCAGCCAAATGCAGGGAAACACTTTATTTTGTTTGTTTTAGGTTTTGTTTTTCCTTGAGAGCCTTTTTTTAGGCCCCAC
AGACAGTGGTGGGTGGGGAGGCGATAGGAAACACATTCCCCAGTGTGCATTTAACATTCATAGCCTACAACCACAGACGTGTCTGGG
TTTTTTTTAAGAAAAGAAATAAAGATTCATCCCCTGGCCTCTACTTCAGATTCGAAGTGGGAGGCAACTCAATGTGCCTGGGGCTCG
CCATGGGCCCACAGAGTCTCGCTATCATCCCCAGCGTCGCAGTGTGGCAGGTGTTTGTAGGCTGTGGGTTCTGGCCACCTCGGGAAA
TGAATGGCACAGGTGAGGGCCTGTGCCACGCCCCACACACCTCAGGGCCAAGCGGGGGCGTGGCTGGCCCTTCAGGTCAGGCCAGTG
GGCCTGGTAGCACATGTCTGTCCTCAGCCGGACAGATGCTTTCTCTCCTGGTTTGCAGCTGTCTTCAAATCCCCCCATGACCCGCTC
TTCCCACTCCTTCAAGTTGCCCTCACAAACCAATGTGGCAAGACTACTGGACTTGTATCCATGGTACTATACTCAGTCCTCTTATCT
CAGCTTGCTGAGGGGCAGGGAGAGGGTCTCTTCCTCTGGGCAGCACTATCTAGATAGGTAAGTGGGGGCGGGGAAGGGTGCATAGCT
GTTTTAGCCGAGGGACTCGATACCGACGTCCCCAGATATAGCTAGGCTAAGTCAAGATCAACAGTCCGGGGTTGGTGGTGTTGGATG
AAACATTCATTTTTACCTTGTGGATGCTAGTGCTGTAGAGTTCACTGTTGTACACAGTCTGTTTTCTATTTGTTAAGAAAAACTACA
GCATCATTGCATACTTCTTGATGGTAATAAATTTGAATAATCAAGATTTCTTACACACTAGGCCTCTGTCTCAGCTCCTGTATCTAG
AGTGTGGTATCTTAAGTCTATGGCTGGGGTGGGTTCTGAGGAGTC
MOUSE PROTEIN SEQUENCE: mP28-021.1 (Seq ID No: 65)
MAPEPAPGRRMVPLVPALVNLGLMAGAHGDSKPVFVKVPEDQTGLSGGVASFVCQATGEPKPRITWMKKGKKVSSQRFEVIEFGDGA
GSVLRIQPLRVQRDEAIYECTATNSLGEINTSAKLSVLEEDQLPSGFPTIDMGPQLKVVEKGRTATMLCAAGGNPDPEISWFKDFLP
VDPAASNGRIKQLRSGALQIESSEESDQGKYECVATNSAGTRYSAPANLYVRVRRVAPRFSIPPSNQEVMPGGSVNLTCVAVGAPMP
YVKWMMGAEELTKEDEMPVGRNVLELSNVMRSANYTCVAISSLGMIEATAQVTVKALPKPPIDLVVTETTATSVTLTWDSGNTEPVS
FYGIQYRAAGTDGPFQEVDGVASTRYSIGGLSPFSEYAFRVLAVNSIGRGPPSEAVRARTGEQAPSSPPRRVQARMLSASTMLVQWE
PPEEPNGLVRGYRVYYTPDSRRPLSAWHKHNTDAGLLTTVGSLLPGITYSLRVLAFTAVGDGPPSPTIQVKTQQGVPAQPADFQANA
ESDTRIQLSWLLPPQERIVKYELVYWAAEDEGQQPHQPLPRR*
MOUSE PANTHER CLASSIFICATIONS
FAMILY (SUBFAMILY)
Unclassified
MOUSE GENE ONTOLOGY
BIOLOGICAL PROCESS
protein modification > protein dephosphorylation
enzyme linked receptor protein signaling pathway > transmembrane
receptor protein tyrosine phosphatase signaling pathway
isoprenoid catabolism > phosphate metabolism
axon guidance > motor axon guidance
defasciculation of neuron > defasciculation of motor neuron
MOLECULAR FUNCTION
protein tyrosine phosphatase prenylated protein tyrosine phosphatase
enzyme > protein phosphatase
protein phosphatase protein tyrosine phosphatase
protein tyrosine phosphatase transmembrane receptor protein tyrosine phosphatase
transmembrane receptor > transmernbrane receptor protein tyrosine phosphatase
protein tyrosine phosphatase > non-membrane spanning protein tyrosine phosphatase
CELL COMPONENT
plasma membrane integral plasma membrane protein
cell > plasma membrane
cell > membrane fraction
cell > soluble fraction
cytoplasm > cytoskeleton
MOUSE PROTEIN DOMAINS (INTERPRO SIGNATURES)
IPR001777 (FNTYPEIII)
IPR001777 (FN3)
IPR003598 (IGc2)
IPR003599 (IG)
IPR001777 (fn3)
IPR003006 (ig)
HUMAN GENOMIC SEQUENCE: hD28-021 (Seq ID No: 66)
GCGTGTGTGGACTGAAACCCAAAAGCCAGAAACTCTCTGCATGTGTCCCTCATGGAGCCATATGGTCTCTAGTCTCTGTTTTTCAAA
ATTTGCAGGACTGATGACTGATTGGACAGTGGTGCAGGAGGGAAGGTCTCTGCATGTCAGTGATTAAATTGCTGAAGATGCCACTGC
CTGAGACGGGCAGTATTGAAGGTAAGCAGATCTGGGGTGGGCGGGACAGAGGAGGCTCTTAGTTATATGGATCTGGAGCTCACAGGA
GCGGCCCAGACTGGAGATAGAGACTTGGAAGTCATCAGCATATGGTTGGTAGTGGTCAAGATGATCCAGAGAAAAGGGGCAGAGCAG
GAGAAAAGAGGAGGCCTAGGACAGAGTCCCGGAAGAGCAGAAGACGAGGTGGTAAAGGAATCAGAGACAGTAGCCAGTGAGGAGAGG
GTGGGCCAATGAAGCCAAGGAAAAGAACTGTTTTAATAACTCAGCAATGGGCAGGAATAATTGGCTGAAAAGTATACTTTTAAAATT
TGGGGGCCGAGAGGGAATCTCTAAATCCTCAAAGTTCTGGAAAAAAAGGCAAAAATGCTTAAAGATGCATAAAATTTGCAATACCTC
CCCCGTGGTGCACCAAAATTACCAATGGTGAAAAACCACTCACTGGAAATATTCATTTTTGATCCGTTTGTCATCCAAACCAAATTG
TCCAGAATCACTGGAGCCAGTCCCATCCCCTCCTCATACGCCAGGGGGCGCACACGCACTGTTTGTGAATTCAAGTTCAGGACAAAA
ACAGGCAGTGAATCAGGTGGTTACCAGAACCTGATTCCTCAGCCCAGATCCCTGAACACCACAGCAACCATGCCCTGCACATTACCA
CGTGATCCCTGAATCCAGGCACCTCAACGATCTCCAAATTTTATTTCTCACCTGACTCCAAACTTACAGGGTTATCAGAAAGACCCC
ACAGAAGAAACCCCACAGAGGAGAAACCTCATCTCTTTTTCTGCTCAGTTACCTGACCCAGGGCAGTGGGGGTAGGTCCCCCATCCT
TCATGGTGAAGCCTAGCAAGCGGCGGGGGGAGGAGGGGTGGGGTAGATAGGAAAGAGCTGGTGTGGCTTTGGCTGACTCTTAAGTGG
TTTTAACACGGATTGCGTGGGGGCGGGGTCTGGAGGCAGGGGCGCTTGGATTGAAGCTCATCAAATCCCTCTGATCAGTAGGTCTTG
GAATAAATCTTCAGGTAGGTTATTGGTTTCACCCAGAAATTTCTAGGGTCATTATTTGAGTTTGGTATGAGAAGTCAGAAACTCAGT
ACTGACATTTAAATTACATTTAGAGGGCATTATCATGTCTTTAATTCCGAGGCCAATGTAAGGTTCACAGCTGGAATTGGTATAATA
TTGGGGTATTAGTCAGTCTCTGCTTGGGGTTGACACTAGGTCAGAGAAGTGGAGGGTGAACATGAGGATGAAGGGGCTTTGTGTGGA
GTTTCTGCAAAGCCAGTCTTATCTTGGGTTCCAAGATTATTATTAAAGCTGGGTTGGTTTAGGGTCCTAAATGGTCTCAAGTTTGGG
TGGGGTTTCAGGTCAGTATCTGTGTTTGGGGGTCCTTTTATGGTGACCCAATCCCAACTCTGGAATTGAGAATCGARTTCAGAGTGC
ATTGGTGTTGATCTGGGAATGGGAGAGCAGTACTGGAGTTTTATGTAAGGCTTGGGGTTCAGAAGAGAGATAACTGGGGTCCATAGC
AAGGTAGCTTTTGGACTTTAGGGTTCATTTAGGGACTGGGGGCTGAGCATTAGTACAGGGTTCCATTTAAGGTCCAGCGTTGGTGCT
AGCGTCACTATCAAGGTTAGGGCAAGTAGAAGTAATAAAGAGATTTAGAGTCAGAATTGGTATTTGGGATCAGTATAGAGGTTCACA
ACAAAATTACTACTTGTGCTAATTTGGGGGCAGATTAAGGGCTTTGGACTCAATATTGGGTTTAGGGCAAGGTGTGGGCTCAGTATC
AGGTTCAGATCTGTATGGGGAGGAAATGGGGTCCTAGAGTGGTATTAGGTTCATGCTAGGGCTGGGAGCCACTAGAAGGATCTGGAT
GTAGTATCACAGTTTGGTCTGTCTATGTTCATTATTGGGGTTCGGGTTCAATATTGAGGTTCCGGTCGGTTTCGACTTGGCGTTGGG
TTGTGGCCTGTCTGGGCCTCAGCGGCTCCGCGGCTCTACGAGCGAGAGTGCACGAGGGGAGGGGCGCCGCCGGGGGCGCGCACGGCA
GGGGCAGGGGCGCGGGCGCGAGCGCGAGGGGAGCGCGCGGCTCGAGCTGGCGCGGGAGCGGCGGGAGCGGTGGCGGCGGCAGAGGCG
GCGGCTCCAGCTTCGGCTCCGGCTCGGGCTCGGGCTCCGGCTCCGGCTCCGGCTCCGGCTCCAGCTCGGGTGGCGGTGGCGGGAGCG
GGACCAGGTGGAGGCGGCGGCGGCAGAGGAGTGGGAGCAGCGGCCCTAGCGGCTTGCGGGCGGACATGCGGACCGACGGCCCCTGGA
TAGGCGGTGAGTGACCCCCCGGCCCCCCACCAGCCCCCTCCGCTCCCGTCCCTTCCCCGCTCTCCTTGCCCTCCCCGCTAGTCCACC
CGGCGGAGCTGGGGGCGGTCCCGGGGGGGACGAGGGCGCCGCTGAGGCCGGGCCGCGCCCCCACCTGCCGCGCCCGCAGCCTCGGCG
AAGNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNCCCTCCCAG
CCGGCGGGGGATCCGCAGCCGGCGCGCCCGGGTCTCGCTCGCGGCCCCGGCGCACGTGCGGTCCCGGGCTGCGCCGGGCGCGAGTTT
GAAGCCCCCCCACCCCCTTCGTTCGCGAGCACGGGATGGGCAGGGGCCGAACGGGCTGTACCCGCCCCCAACCGGGTGCAACGCCCT
GGAGGCCCTGGAGCGACCTGTCCCGTGAGGACGTGGCCACTCGGACCCTCTCCGAAAGTTCACTCAGTGGCCGCCCCGCGTCCCGTC
CCGTCCCGTCCCGGCTCCCCATCGCCGTGCCGGCGTCTCTGTCTCGGCTGCTTTCTGTTTTCCTCGGCGTCTCTGCCTCCCCGTGTC
AGTGCCTCCCAATCTCACGCCCCTGCAATCCCAGGGTCTCTCCAGCTGTCTCCATTCTTTTTCCCCAGGTCACTCTGTTCTTTCTCC
CCAGGTCTCTCGATTCTGTCTCCCTGGGTCGTCTTGTTCCCTGTCCCTGAGTCTGTTCTCTCCGCTTGGGTCTCTCTATTTCCTCTC
CCTGGGTCTCTGTCTCCCTCCTTAAGTCTCTGCCTCTTGTCTCTCACCCATCTCTTGGTGTCTCTGCACTCTCTGTGCTAGGCGCCC
CCCCATTTCCCTGGAATGCTGCCCCTCTGGTTCTTCCACCCCGGAGGGGGAGGGGCTAGACTTTCTCCCCATTTCAAGCCCCCCTGC
CCCCCAGTGCACGGCCCCTGAGTTGGAGCAGGTTGGGGGTGGGGAGCGCTACCTGCCTGAGCTGTGAAATGGGAGAGGGAGGCTGCC
CCAGCCTGGGGGTTGTCTGGATCTCCCTGAGGGGCCACAGGGCAGGGCCGGGAGGCTGGATTAGGTTGAGGAAGGCTCCACTTTTGA
AGGAACAGGGGCAGGGATCCAGAGCGATCTGGTGTCGGATCTGGCCTGAGAAGCAGGGAAGTGCACTGGGGTGAGAGGCGTAGAAAA
GGATGTGGGGTGGTGGTTCCAGGCGGGTGCTCATGGGGAGGAATCTGCTGAGAAGGGAAGATCTGGTGGGGAATCCTTTCGGAGGCT
TCGGGGATCTGAGGGATCCAGAAGAGAGTTCTGACAGGCTATCTAAGGAAATTTCTGTGCTTAGCTCCTTATTAACTCTTGCTTCAG
GGAGCATGAAAATCCCTGTTTTTTACTGATTATATGAATGAGCCTAGGGCTTCAAGAAGGAGCAGGTGTAACTCCCCAAGTACAACC
CCCTACATTCTCAGCCCCAGGCACAGGCCACACTTTCTCGTACCCTCTTGCCTTCATTTCCACACTCAGCTCACCCCCTCCACCCCC
ACACACACTCCCTATTCAGCTGCTTTCTTGCCCCCTCTCGCATACCTGGGCCCGCATGCCGGCCAGGGCTCAAGAAATCTGTGCCTG
CACACTCTCGCAGCATACCTGTGCACATGCACAGTCATCCTTGTGTATTTTTGTATATGCACTTTCTTGCACATTTTACTTGCTCAG
TCTCTTATTTGAAATGTCTTTATAGTGTTTTTTTTAGTTTCCTTTATAGTTTCCTTTTCCAGCTGCTCTTCCTCTTCCCTCTTTCTT
TCCTTTAACAAGAAAATTGTTTTAACATAATTCCTTCCTTCCTTCCTCCCTTTCTTCCTTCTTCTTGCAACTACTGATACCTCCCCT
TTATCCAGCTTTGTGTGCCCTGGGCGGGTTGGCTTGGTGTACAAGGCTGCCAAGAGAGGAGGGGGTGGTTGGGTGTTGGTGAAGGAA
TTTGGCGGGGCAGAGTTAGACTGCAGCAAACGGAAACTAGCTACACATCTTTTACTGGGAAGTGTATGGATACGCAGCAAAATACTA
GCCCTGGCAGTTTGGGCACAGTCCACCCTTTTTTCCATCAGCCTGAACTATGGGAGTCTAGCTTTGAGGGTCCTCCCTGGGGGCGGG
GAGGAGGGGACCCAGACCTCCTGCAGTAGAGGGGTTGTGATACACACCACGGCTCCTCCACTTTCCAGCTTCAGGCCTCAAAGAGGC
CCCTTTCTCCCTGAGTCTCAATTTCACTCCCTGTAAAATGGGGTAGCTAATCCAACCCGGACTGGGTCCCAGGGCTGTTGTAAGAAT
CCAAGGGAACTGTGTGGCAGTGCATCGTCAGCAGTGGCAGTCACTTCCCTTGTGACTGACACTGCTGTGTGCTTGGTTCCAGCTTGC
AGTCAGGGCCCTACCCTCGAGAAGCTCGTGGTCCTTGGGGGAGCAGGTTGCACCATGGGATTGCAAAAGCAATTCAGTTACCTGTCA
TTGATTGCTGACTGTGTGCCAGACCCCGGGGTAGGCGCTTTCGGCCCCGGCTGCCTTTCTTCCTTTGGGACTAACAGCTATGTAGAA
GAGGCAGCCCACCCCTAAGGTTCAGGCATCTGCTCAACTAAAGCAGCTGCGGTTCTGAGTTGGGTTAGAACATTGCAAGAGTTCATC
AAGAGACGGGGGAGAGTTCGGAGGTCAGGGTGAGGTGAGTCCCAGAGGAGACCAAGAAGAAGTGATGTGAACTAGGTTTGGAGGGAT
GAGTAGGAGTTTGAAGAAGAGAGAAAGGACTTGAGATAGATAAAAACCAGTATGAATAAAGACTGGGAGGTGTTGTCACTTGTGAAG
TATTCAGAGATGTGGCAGATCACAGAGGGCATTGAATGCCCTGTTAAGTACCTGTAATTTTACTCTGAGAACCCAGAGAGAAAACAC
CAAAGGTTTAAAGCTTGGATAGGGAGGGTGGAGTGTGCTGTGGTCAGGTTTATGTTTCCAGAAAATGCTCTGTGGCTATCGGAAAAG
GATGGAGACAGGATGCCCAGTGGGGCAAGAGGGTGCAGGTAAGTAAGGTGAGAGAAGATGCTGGCTTTGATAGGTGGTAGCATGGTG
CCATCTTGAAGGTACACTTGTCAGGACAGAATGATTTATTGGATGTGGGAGTGAAGGAGAGGAAGAGGAAGGAGTCCAGGTTCCTCG
GAGTGGTTGAGCGGGCCTTCACTGAAGGCCCTGAAGCTTCACTGTGTTAGGGACTTGGTGAGGAGGGGCAGAGAATAAGCTCTTAGT
TGGACATGTGGCCCTTGAGGTACTTGGGACATTGGAGGCTCAGTGGGGGAGGCACATCAAGGAGGGGCTTTGGTGTGAGGGTATGGG
AGAGAAATGTCGGTCGGACCAAGAGAAGCGAGAGCTGTATGAGAGACCATAGAGCTGTGCACCTCGAGAGAAAGTAACCTCCGACAG
TGGTGAAGAGCTCGAGTTTTATACTGCTAGGATTGGACTCTGTTTACTAGCAGCGTGACTTTAAGCAAGTGGCTTAACCTCTCTGAA
TCCGCTTCCTCATTTGTCAAATGATTATAATAAAAACCCGTGCCTTATAAGGGTTGTTATGAGGATTAAAGATAGTATGCATAAAGT
GTGTGTAGCACAGGGCTTGGCATCTAGTGAGTACTGTTGTCTGTGTTTGCAGTTACTGTAATCATCCTCAACTGGGATGGTGTAAAC
CCACCCTCCCATCAGGGGTCACATGAGGTTGCCTTTAATGAGAAGATAGACTGCTAAGGTTAGGGTCCCTGTCTGCCTAAGGTTGCT
GCAATTCTGAGCTGAGATTAGCAGGTCAAGGAGCTCAACAAAGGGGGTGCCTGCCCCTATTGTTATCGATGGGATTGTTTGTGTTGC
TCAGGTGTTTGACCTTCAGGTGGGTGGGCTTGAGAAGACTAGAGGTGAGGCCTGCAGAAGAGACTCCATGTTTGGGGTAGGTGGAAG
GGAATTTTGAGAAGGGGCAGCCTTGGGGTATTGAGTTCTCCATTTCCTCCCACCCTCCCTGGGCCCTGGGCTTCTACTGAGGCTCTT
TCTGCCACCATCTGCCTCTGTGGTCCAGATGCACCATGTCTCTGTGTGACCGTGACTTCTAGGTCTGAGAACTCTCGTTTTAGCTCC
TTAGCAGTATTTTAGCTGCTGTTTCTGGGGACTGTTCCTGGTGCAGCCACAGGGGCATTCTGGGGGTTGCTCCAGAGGGCACCTGTG
CCTGCATTCCCCTAGGTATCATCGACTTGCTCCTGACCCACTGCCGGGCCAGGTTTCAGGCCTGGCAGATGATGGGGAACCCTACTG
GGGCCAGCCCTGCGTTGGCCCCAAGGGCTTTCTTTCTTGATATTGGACATGACTGTGGACTTCACCCCTTCTGAGGCCTTCCTGGGT
GCCAAGCAGTGTGATTTCTTTCTTTTTCTCTTTGGTTTGAAACAGAATCTCACTCTGTTGCCCAGGCTGGAGTGTAGTGGTGCAGTC
GTGGCTCACTGCAGCCTCCACCTCCTGGGCTCAAGTGATCCTCCCACCTCAGCCTCCTGAGTAACTGAGACTACAGGTGTAGTCCCA
CCTGGCTAATTTTTGTATTTTTTTAGAGACAGGGTTTTGTCATGTTGTCCAGGCTGCTCTTGAACTCCTGGCTCAAGTGATCCACCT
GCCTTGGCCTCCCAAAGTGCCGGGATTACAGGGGTGAGCCACTGTGACTGGCCAGCAGTGTGATTTCTAACTTGGGTCCTGAACCAT
TCAGGCCCTTTCTGAGTAGCTTTCTAGTCAACCTTCCAGATGTCAGCTCTGGCTCTGTTCTGGGGGTGTATCTGGTTGTCCTGGTTG
TTCTGGGCTCTGTCCCAGTGTGAGTGTAGAGTGGGTTCTGGGAGCTGCAGTTGGTGAATCTGAGTCTTTATTTGGTTGCCTGATAGG
TTGTTTTGAGGTCAGTTTGATCCCAGACTGACGTGGTTCAAAGTTACTGTTTGGCTTTTCTAGGATCCTGCCCTCATGCTGTTCTGT
GTGCCTCAGGGTCTTTGCAAATGTTTAGTCTTCTAAAATGGCCCCTGGCCACTTTGGGTACACACCTCTTTCCAGGTGTGGCTTCTG
CTCCATGTAGGGCCTGTCTCAGTCTCCAAGCACGAGACCCTGCTGTGGCTGGGTGTGGCAGCAGCGCCTGGCCAGCTTGGGGGGCCT
TTCCCATTACCCAGTTCCCTGCCTTGGACTCTGCCCCTCTGCCCAGGGTCTCTGCACAATGGTGCAGCCAAAAACCTCACTTGAATA
CTGTGAGGCCCTGCGGGCATCTCTAGACAAATGGGCCTTTAAGTTTGGGGCCTGGAAGCTGGTTTGTCTGGCCAGACCTGCTGGGCA
GCCTGTATGCTGGGAGCAGCAGCTGCTGGAAACTCAGCTTGGGGAGCGGGGAGGGAAGCATGCAGAGAGGGCTGCGGCTTCTGGTGC
AGGTAGGTGGGGCAGTGGAGGCTGGGTCGCCAAGCTGGCGAGGGCCATCTTGGGTCAGTTGGGATGGCCTTCAGTCGGTGAGAGTAC
CCCCATATTGGCAGAGGTGGTTAGTGCAACTTGAGGGTAATTCCCAGGTGCAGAGGTGTGTCCTTGATTTCATGATGAGGGTCAGTC
TTGGGCCGGTGAGGACATTCCCACGCACCACCCTCCCACTGTGGTTTCTTTGTGTCCGTTATAGGAGGAGCCAAGGTGGCAGAGTGG
TGTGGGGTGGGGATCACTGTGCATGTGGGGGCTTGAGGTGAGAAGCCTGCTGCAAGGTGCAGCTTGCGTCGAGTAGGCCAAACAGCT
GAGCTTCCGTGAAGTGGTGATGAGGTGGTATGGGTGTGTCTGACCCCCACCCCCACCCAGGATGGTACCCACGCTGGTGGGGACTCA
ATAATGGCTCTAGAAGGAAGGGGCTGGGCTCTGTCGCTGCATGTGTGCCAGCAGAGGAGGCATCCAGGGGCACGCCTTCATTTTAAA
AATTATAGAGTAGAGAAAGTCAAAAATAAATATTGCTTCTGAAGCTTCTTTGAAGACTGGCATGTATGTTGGGGTGTGTTCTTCCAG
GCTGGGCTGTTTTCTTTGAACTCATTCATGCGTTCATCCACTCATTCAGTGTATGCTGACTGAGCACCTACTATGTGCCAGCTGTCA
GCCAGGCGCTGGGGATACGGAAATGAGCTGGGCAGCCACAGTCCATGCTCCTATCAAGCTTATGGTAGTGACTCAGTAATTGTAAAA
ATATATAAATAGTCATGTTTGTGGTCACGGCTCCAAGCCAAGGTCCAGGCAGGTAGTCTGGGACATTGTGGAAGGCTTCCCTGAGAA
AGTGCCACTGGACTTGAGATCCGAGGGTAAGTTGGCCAGAAGAGCATGGGTAGCCTGTGCCAGGCAGTGGCATTCAGCACTGGCAAG
GCTGGGAGACAGGAAGAAATTTCGCACATTTGAGGATCAGAAGAGGAAGTTCCCGTTCTGGCAGGAGGAGAGCATGGGCAGGTAGGG
CTGCAGAGGTGGGAGGGGCCGACTCTGCTTGGCTCTGGGGTCATGCTGAGGAGCTGGGCCACAGAGTCTACTGCTGAGGTCAGGGAC
TTGGTCTGTGTGGCTCACAGCTATATGGCTGGTGCCTGGAAGGACGCCCAGCACATAGTAGGTGCTTAGGGAGTATCAAAATGAATG
AGCGAGGGAGTGAAGGGAAAAGATGGGCATTTTTACCAGATGGGGATGGTTCTACATGAACATTTCCATAACCTGCTTTTTTTTCTG
CATAAACAGCAAATTTTTAATGTCTTTCTGTGTCAATACATATGCTGTTGTGATGAAATTCTTGATGTTTTCTCATGCTTTCCTTTC
TGTCCAGGAAGGAGTGGAGGCCCTGGTGCCCGGCCCTTGGTGCTGAGTATCCAGCAAGAGTGACCGGGGTGAAGAAGCAAAGACTCG
GTGAGTGTGCCCCACAGAGTGGCCAGGAGCAGGGGTGCACAGGGGTCCTATGGACCAGGCTGAGCAGCTTGGATTTGATGCAGGGTC
AGTGGAGAGCTATAGAGGGGGTGTCAGCAGGGGGCATGACATGATCCAGTGTGCACTGGTGAGAGATTGTGGAGGCCTGGACCAGGG
AGGCAGTGACAGAAGTGGAGAGAAGGGATGTGAGTGCTATTTAGGGAGGTAGAGTAGACAGGACGTGGTGAAGGATTGAATGTGGGG
CATGAAGGAGGGGAGGTGTCAAGGATGGTGCCTGAGTTTCTGGCTGGAGTGGATGATAGAGGTGGATGGGGAATGACATACAGCTCG
TCAGCTACTGTAGGTGTGAAATAGGCTTCCGGGTGATGTCCTGGGACTTACACCAGGATCTTTCTTCTGCTTCTCCATTCACCACGT
TTCTGTAGAAAAGACTGTTCCCATCGACTGTAGGTTGTCGTAGAAAGAGCTTGGTGCCTGCAGGATCTCGGCATGTCCTCTGCATCT
CTTTGATGTCCCCTTGTTCTCCTCTCTGTCTGTTAGAAGCACAGAATGGGTGGGCTGGAGTTGCAGGGAATGAGGTTGGTAAGTTGG
TTTAGAGTCAGCATCATGGGCCTTGGATGTCAGGCTAAAGACTTTAGACTTTTTCCTGAGGGCAATACAGCAGCATTGAAGGTTCTT
AAGCAGAGAAGCAATATCCATGTGTTGATCTGTCCATTGCCCATCCGTTCATTCTTTCACGGAACACTGTCTGAATGCCTGCTGTGA
AATAAAGTGGCAAACAAAAGTGCAGATTTGCATTTTTATGTGCTGAAAGGTGGCACTGGTCTGTGTGCGGGATGGGTTGGGAGTGAT
TGAAGGCAGGAAGCCCACCAAAGAGGCAGGAAGTGATGAGGTCTTACTAACACAGTGGCAGTGAGGATGGAGGCAGGTCAGATTCAA
ACTTATTGATGGGTTAGATGTGTGGGATGAGGACAAGGGAGCATTTAAGGAGGGTTTGCGGGCTTGGTGACTGACAACATGGTGGTG
CTGTCCCCAAGACTGGACACCCTGGAGGAGGAGTGGAATTGGGGCAGATGAGTTCTGGTTGGGCCATCCTGAGCCAAGAGCTCGGTG
GGTCTGTTCACCCTCTGTAAGCTCCTCGAAGGCAGAGCCTGTGACTCTCTTGTTCATCACCCTGCCCACTGAACATCTGGACGGGTG
GCTGAACTAACAAGGCTGTGGAAGAGATGGGTCTGGCCTGGGTAGGGGAAGGAAATGTGTATGTCCTGGGTGTCTCCTGGGGTCCAG
ACCCTACGTTTGGTGAGATGGGCACTGGGGTTATGTAGAGGGTGCAGCAAGAGGGGCTGTGTCACAGGCTGGAAAGGTCAGATGGGG
AGGGTGGGTGTAGGGACACTATGTGTCCCTTTGCCTCTCCATCCTCAGATGATGGTCTGACCTAACTCGAAGTCCAGTCTTGCCAGA
ATAGGTACCTGAAGGTGGAGCCCTCTGTCCTCTCGGGAGGCCACTAGCTGATGGCATGTCTGGTGGGGTGCAGATGGGGTGTGCTAT
AGGACCTGACTTCTGGAGGCTGGGTAGGGCTGATGTGGGGGTACAGGGGAAGATACTCTGAGATCCTGAGGCCACGCGCCAGCAAAT
ACAGGAGTTAAGCCAGGTTGGAGCTTCCTTGGGTACAAGGCCCAGGGTGCCCACAGGGGATTGGGTTCTGGGGCAGGGGCCAGGTCA
GGCTCTAGGCTCAATTGGCAAAGGATCTTGTTGTCTGTGTTGGGGTTTCTCAGCCTCAGCACCATTGACATTTTGGATCAGATAACC
CTTATTGTGGGGGCAGGGCTTGTTATTGTAGGGTGTCTAGCAGCATCCCTGGTTTCTTCCCCCTAGATGCCAGTAGCATCTCCCCGC
TTTCCCCAGTCTCGACAATCAAAAATGTCTCCAGTCGTTGCGTAATGTCCCATGGGGGCAAAATTACCCTGAATTGAGAACCTCTGG
TCTACAGCCTCTTGCTGCTTTCACTATACTGTACAAACCTGGAAAATTGGGGAGGGTCAGTGGGGAAGATGGATAGTGATGGGCCAC
TTGAGCAGAGTTCTGAGGGGTGAGTGAGTGCTGGGGCAGGAGAAGGACATTTTTTTCCAATAGACAGAACGGCAGGTACAGAGGCTT
GGAGGCACAAAGTTGGGAAACAGTAATCCAAGGGGGACCCCAGCCCCAGAGGAAGGGGCCTGGAGGCTTAGGGGTTACAGCCGCAGG
AAGATACCTCTGCTGGTCCCCTTTGGTCATTTGCCTCCAGAGTTAGTCTCCTGCTGACCCTTTGCCGCCTCCAAGTCTCTGCGACAC
TCCTCCTAATCTCCCCCTCCACTCCTCAGTGAGATGGAGAAGCTGAGAGCTGCAACAGGGTGGGACTCAGTCCAGCTGGTGGGAGCC
CCGATACCAGCCGTGGGAGGGAGGGCTGTTCCTGGCTCTGCTCACGGATTCTGGCCAACTGGCTGGAGGAGGGAAGGCGGCCTCACC
CTCTTCCCCACAGGCCCCTCCTTCCCTGGGTCAGGGGTCCTGGCTGAGACTATAATTTATTCCCGTCATAATCCAGTGGTTGGTTTG
GTGAGAGCTGGAAACATGTTGGTTTTCCCTTCCCAAGTATAACAAGGCCTGTTTCCGCCTCCGCGGCCGCTGCTGCAGTGCCACGCG
GTGAACTGTCCAGGACATGACAAAAGGGCTCGGTTAGCTGCCCGCTGGTTAGAAGATGAACGGCTCAGAGCTCTCCCTGCCGACAGG
CTCTTCCCTTCCTGTTGCCCAAGTGCTGGCCTTTCCTCTTGGCCGTCTGCTCTGTAGGGCCCTGGATACCCCTCCTTCTGCTCATGT
CCATTTCTGGGCCCCAGGGTCCTGGCCTGGGGAGGTGAGATGGGGGAGGCTACAGAAACAGGTGGTATTTGGAGACTAATTAATATT
TGTGAATAATAATGCATGCCCAAGGAAAACAAATCAATCTGTGTTTGCGTATTTATGATGAGGACTCAGGAATGATCCCAGCTGTTC
TCCAGCCAGGGAAGCCCCCATCACATGGTCCCTTGGGCCCCCACAGAGGCTGGCAGGGTGGTTAGGGTGGGCTGCATGGAGGTGACA
TGGCTTCTGTGTTTTGCAACGTGAATCTTCTGTGACGTTCATATGCGAATGCTGTGGCTTGGTCTAGTGCCTGCTTCTTCCTTTTCC
CTCAAAGCAGTGATTCCCAGGCTCTTTGGTTTCACGAACCAATACAATTTCCAAAAGTACTCAAGAGCCAGACATGGGGTTGGCAAT
TTTTTATTTTGCCAAGTAAAGGCATTATAAAACAACTGCTATCTGCTGTCCCCATCATTTCATACAGGAAAAAAGAACATTTTAACA
CCAAGGACAATATTTGAGAATAAAGCAAGAAAGGGCAGTTGGCAGCTTTACACTGACGTAGCAAAGGAAAAGGATATATTAAAACCA
TTCTGAATGTGGAAAAATTGTGTCCAATTATATTCTATAATATCATACCGTATGTAATTGTGTTCCTGCCCAAGTATGTCAGCTTGG
CAGAACGGATGGTCCACAGTGCAATTCAGAGACGAGCTTGAGTGCCCCAGTGCTCCGGCCTTGTTTTTATCTCGGTTGTTGAAAGCC
CAGGCTCACCCAGCCATGCTGGTGAAGTGGGTGCTCTGCTGTGAACACCGTTGCATGGATTCACAAGATGCTCAACCTCGCAACCGC
AGTCAGGCTAGCAGCAGCCCTGCCCCTTGTTCCTCTCCCACCTTCTCCCCACAGGTGACCCTGGGACCCTGGGACTCTGGGGGCCAA
GGTCTACTCTTGAGGCCAGGCCTGGGGATCCAGGGCTGCTCTGCTCTGATGAGGGAGGCCCTGGAGCTGGGAGTGGCCTGAACCGCC
TGACACCAGGGCAGGCTCTGTGCCCGGAGTCTCAGGGCGGGAGGCAGCTTGCTCTCGCCAGGAGCTGGTGAGGGAGAGGCCCTGGCT
CGTACAGCTGTGTGGCTGCCAGAGTAGTTGCCTGAGAATGTGTTTGTGTGTGTCCCTTGTGTTTCCCCATCTCCCTGGGCATCTGTG
TCCTTGTGTCCATCATGGACCACTGGAAGAAGCTCTCTGTTCTACTTTCTGGATCTGAACAAAGTGTCCTGAGCACCCCAACCCAGA
TACACAGGGGGTTTCTGGAGGCCCCACGTTGGGGGTAGGGTTACCTGAGGCCCAAGTTCTCCAACACCCATGGCCAGACTCTCATCC
AGGCTTGTACCACATGGGTGTCTATTCTGGGAGTTTTACCCCCATGATGGCTCTCCTGGGAGCTCTGCCCCCACCATGAAGTCTGTA
ACAGCACGTTACACCTCTGCTAATGTATGGGGAGTTGCACCCCTGTAATGTCCTTATCAGGCAGCTGCACTCCATGATGTCTGTACC
CTCTGTGTCCGCCCACGTGATGTCTAGACCACACCATTACACCTCCATGATGTCTGCACCTGGGCGTTATACTGCTATGATTACTGC
ACCAAGGCGGCTACAGGGGATTAAGCCTCTGCTCATTGTACTGGGGAGCTATATCCCATGATGACGGTGCTGTGCATTTTATTTTCA
TAGCAGGGAGATATGCTCATGCTGGGGGCCTATGTCCCTATGAAGCCTGCCTGGCAGGGGTTGCATGCTTGGAATGCTTGTTCCTGG
AAGTTAACCTCTGTGACTATTATATTGTTATACCCGATGATGCCGGTACGAGGCCACTGTGCCCTGTCCTTTGGTGCAACACAGCTG
TTGAGTTATAAACTCATGTGTCTCATTCAGGGGCTCCAGGATGGTCCATCAGGGGACCATACCCCCATGATGACCTGCCCTGGCATA
GGATACCAGATGTGTCCACAGGCCCAGGGAGGAAGAGCCACTCATAGTCCACCACCTGATGGCCGCTGGGAAGGTGCTCCATCGTTG
GCTGCATGTGGCACCATAGCGCAAGCTCAGGCAGGGCCTTGGTGATGTGTGACCTCCATCCCACTGTGGCCTTGCAGGCGTCATTGT
GGTCTCAGCCACCTCAGCTCTGGGATTGTGGAGCAGATCCATCCCCAGCTTGGTGTAAATGGGGCTGGACACCTATCAACGTTCTTA
CTTGAAGAACTGGTGTAAATGAAGGCTGGACACCCTTCACCGTTCTTACTTTGTTGTTCAGCTCCTGTCTCAGATTTTTGGTGGGAT
CCTGGGACAGGGGACAACTCTCACCCCTTTTCCTTTGTGTTTGGCCCCATCCCACTTTGCATTCCCACCTGCTTTGTCCAACACCTT
TGCTCAACTGCTGCCTTCTGACCTCATGAACTTTGATTATCTCAGTTGAGACAAGGGTTCAAATTTCAGAAACGACTGGAGCACCTT
TGAGCCCTGCCCATTCCTGGTCACCTGCCCTGTCACCTTGCAAATTCTCTTACTATTGTGGGTCAGCGGGGACCTGGGTCTGATCAC
AACTCCCCTATCAAAGTCCATGCGTATCACTGTGGGGCCCCAAGGCGTGTTCACCGTCATGCACTCTGCTCTGTTGCGTCTTCACAG
CATTCATGAGGGATCGACCACGTCTCACCCCTCTGCCCAGAGCATGGTGTGATCAGAGCTTGTGGGCCCTTCCATACATCCCCTGGT
GCCCCCCAGGTGAGGGGTATGGGAGAGAGACCCTGGGGAGCTCACCAGTGGGGCCAGGGAGTGATAGCAGCGTATCTCCCATGTATA
GTGCGGCCATGCTAGAGGCTTCACCCAGGTGCCTGCAGGCACTTCAGGCCAGGAGCCTTGGAAGTAAAGGCCTAGGGGCATTCAGCC
AGTCCCAGACGCTACTCAGTCTTTTTGTGCATAGCTTCCTGTCTCAAACATCAGCCCCTGAGGTTCACACCCCATCATCCCTACACT
TGTTTGGCTCCGGGCTTCTGGGGCAGGGCAGAAGAGATGTGAGGTTTGAATTCTAGGGTCTAACACCCCATTCCATGGCCACTGAGG
CCTCCCTGTGGTTTCTCTGTGAAGTAGGAAGGTGCCATGGGAATGGGTAGGACGAGGGTGGCCAGGCAGGGCAGCAGCAGCTTGTCA
AGAGCACGTCCTGGTGAGCACTGAGGGGTTGTGGCTTTGGCTGGAAGCCTCTGATACCTGGAGCCTGTCTTCTGCTAACAAGCCTGT
GTTCAGGGGCCTGTGCTCAGCGTGCTCGTGGAGTCTGGCCTCCCACCTTCTCCCTCCCTGCTGGGGAGAGGAGGGGCAACAGAATTC
TACCAGGGAGACTCCAGAGTCAGTTGGCTTTGCCCCTCACTGCTCCTTGTCACTGTCTTGTCTCTCTGTCCTCTGTCCCACGTAAGC
GTCTCTCTGACCCTGTTTCCCTGTCCTCTTATGGGGGTGAGTTAGAAGCTCAAGGTTTGGACTGGAACACACTGTACATCCTCTGGT
CTCACTCACTCCCTGTACCTCCTACTCAGGTTTAGAGAATGGCCCGAAGGCCTCCTGCGCTCTTTCTCCCTCCACCGTGACTGTTGG
GCTGTCCTTGTGGGTGCTGGGGAATAAGTGAGGAGCTTGGGTGGTCACTGGGGTCAGAAGGTCTAGGTGGTCATTGCACAGCCCAAG
TGGGGGGCTCTGTTGAACTAGGCTCTGGATGGTCAGTGAGGATGACTGGGGGTCAGAAAGCGTCAGGGGTGGGCATTAGGGACAGCA
GTAAATACCAGCTAACTGGCTCTGCCCTTCTCTCCATTACAGGTTGATTGTCCTGGGCTGTGGCTGGCTGTGGAGCTAGAGCCCTGG
ATGGCCCCTGAGCCAGCCCCAGGGAGGACGATGGTGCCCCTTGTGCCTGCACTGGTGATGCTTGGTTTGGTGGCAGGCGCCCATGGT
GACAGTAAGTCTGACCCCTCAAGGTACAGATCTCCCAGGTTGAAAGGTGGCATCCTTCCCAGCAGGACTCTGGGATGGGGACAGACC
GGCTACTAGGAGAGACAGGGTGGCCAGAAACCCTTTAGACCTTCTGTCCTAGAGAGCCCCCACTGCTTGGAGAGCCTCCTTCTAAGG
AATACCTGGGTACTTAGGAGCATCTTTAGGCACCAGAGCGGGGCAGTCGAATCGTGACCCTGTCTTATGGGGCTGGGGTGAGACCTG
TCCTGGATTCATGTGGTCATTGTGTGATGTGCGCGTGTGTGTGCTGTGTGCACTGTTGCAGATCTGTGCTGAAGGCACAGTGTGAGC
TACAGCCAGGGGTCGGCATGGGAATGGGGGGTGGCACGCAGGGCATGCGTATGGATGTGTGCCCAGGACGCGGGTGTGTCCATAGGC
CCAAGGAGGGTCAAAAGCGGGAGCCACCCATAGCTCTTCCCCTACGCTGGACCCTCAGGATCCAAGTATGCAGATCTGAGCCCCTCC
TTCTCCTTTCATGGGGACCTTCTAGGCAAAGGAGCATCCCCCCACCCCAGCTCCTCAGACCTGGAAATGGGGGGAAGATTGTGTTTG
GCCGGCAGGAAGCTGAGCAACGCCCCTCTGTGTGTGTTTGTGTTGGAGGGAGGTGGAATTGTCCCCATCCAGATCCAACTCCGAACT
TTGGTCCCTTTCCTGCTGCCCTTCCCCCTGCCTGGCACTATGAGACTGGCCTCAGCCCGCCCCATCGCCATGGTTACCACTCCTTGG
TTACTGGTGGGAGGCGGGGCACAGGGCAGATTTAGCCAATCTGCAGGCTGAGGGGGAGGGGCGAGGTCGGAGCCAAGGTCCCTGGGG
GAAGGGGCCGTTCCCAGCCTGTCCAGAGCCCAGAGGTGATCCAGGGCCAACCCTGGGGTCAGCCCACGGGTCACCAGGCCATAGGGC
TCCCCCACCTGCCTCCGTATCTCTGGGTACAGATCTCTCCCCTTGCCCCACCAGGGCTCTTTCTCCTAATGGCTCATTAATTATTCA
GGAACTGATTCTGGAGTGGGGTGGGACTGGTGTGGTCCCGCCCTTTGTCCTCAAGTGCTGGGTCCTGGGTGGAGCTAGAAGGGAAAG
GAGAAGGGGCAGGCATTCCCAAGGGGTGGGGCAAAGGGTCATGGGAGCACCAGGTACCAAAGAGAGGGCTGGGCAGGTGAGAGCAAG
GAAAAAAGACAGGTGGGGAATCACATGGGGAAGTGGGGCAGGGGATCTCCAGGCCTGGCTGGAACCTGCAGGGGCAGGTTGGAGTAG
AAAGCCTTATCAGGTGTGACCCACATGGTTGGCAGGAAGGTGAAGGCTTGTCCACGTGTGGTCAGTAGCTCTTGGACATTGAAGTAC
TGTCCTTGCCCTCCCCGCCGACCCAACCCCAGGCACTTTCAGGGCCTTCAGACAGGAGCTCTAGGGCAGGGGAAGGATGGCCCAGTT
GGGCTTCAACAGATAACACATCCTGGGAGAAGAGCTGCCCTCCTCCTCCTGCCTCAGACCCAACGCGCCCCAACCTGTACCCCTCAC
TCTCATCCCCCAGCCTGCTGTCTTCCCGTGTTCTCTCCCGTGAAAGGCATTACCACCCACCCGGTCACCCAAGTCAGAACCTGGGCC
TCCTCCAGCCTCCTCCCTCCGTGATATCCCACATCTAATCCATCACCAAGCTCCGTTGCTTCTACCTTCTGAATATCTTTGGAATGC
ATCCACTTCTCTCTCTACCGCCTTCATCCAAGCCACCCTCGGCTCTTGGGCTTTTGCACGCAACAGCCTCCTGACGGTTTCCCTGCC
TCCAGTCTTTTCCTTCCTAATCCATTCTGCATTCCAAAGGTAGAGTCGTGTCACAAGGTGAAAACATCATCCTGTCACTCTTCAGTT
TAGAATCCTTCATTCCCTCCCACCCCCAGGCCCCCAGGGTCGTAACCCTTCTATAGGCCCTGCATTATCAAGCTTCTATCAACCTCA
TCCCTCGCTGTCCCTCCACCCACCCCACTCACCCACCACGCTGACTTCTTTCAGTGGTTCACCAATTCCCAGTAGTAATTGGTAGGG
GCAAGGGGGAACAGTTTTTAAAGCAGAGAACCTCCATGCTGGTTCCTTAGGAACCAAAGCCCAAACACCAGGTCTTGACGGTAGAAA
GCAGCTGAAGAAGCTGAAATCCTGCCTTCTTCAGGTAGGGGGTTCAGGGGACTCAGGCCTTCTCCCAGGCAGAGAGCCATGGGGCAG
GAGCCTGGGCTGGGGGTCAGAGTGACCGAGCCGCAGGAGCCAGGGTCTGTCTTCCAGGCCTGTCTTACACTGCACACAGTTTTGGAT
CTCTCCCTGCACTGAGGCAGGGCTCAGGCTGAGCTTGGGCAACTACTAGCCAGGGGCAAGTGGCCATACACAGACAGGGCCACTGCA
GAAGATAGGGTAGGCACCCCAGAGAATTGACAGCTGGGGCAGTGAAGCGCGGAGTGGACAAATGTGTTTCAATAGGCCTCTTAACAA
GACAAATGAATGGGGGCCCTGGCCTCTGAGGGGAGTGGAGGGAAGCGGGTGGAGAAGCAGCTGCCAAGAGTTAGCCCAGAGGCCCCA
GTGTCAGTGCCGCACGCGCAGCTCCAGTGGAGATTTGGGCACACATTGGGGTAGGATCTGCTGCAGCGCAGCTTCCCCAGCCAGGCT
TTGTGGCTTCTCAGGAGGGGAGGTTGTGGGGCCAGAGGTGTCCTGAGCCAGGGCAGAGGTTTTTGCTGATCTCAAGTGCGTGTCGCG
TGCCTGTCTTCAGGCAAGACCTGCAGTGTGGAGGCACAGGCTTGTGAGCAGTGACCACAGGGTGTGCTGGGTAGGTGGCACTGACAC
AGGCCATGGCAGAAGCATCTTGGGAGAGGAGTTGGGAGGCTTCCTGGAAGAGGGGAGCCCTGAAGGGTGAGTGGACATTTGCTAATG
GGGGGGTATTGCATAATGAGGTGGGGTTGGGGGGAAGAAGCACGAATGTGGCTGGGCTTCTCTGTGGAATTCATGGGAGAGCCACAG
TGAGAACCCGACAGCATGGGACAGAGGTGGGGTCCGAAGACCAGGTGGGAGTGAGACCCTGGTTATGGCCCATTTACCCAGAATCCT
AGGAGCTCAGAGCAGGATGCGTGCTGGCTGGAGGGGTGGGTGGCGGGTGGGTTGACAGAGGTGGGCAGAGGCTGAGGAGCTGGGAGT
GTGTCTGTTGTGTCATTTCCCTCCTCCCCAGAGCCTGTGGAGCACACAGGGTCTGTTGTCTGTCGTCATGCTCTCCCCCTCGTTCTA
TGGGTGGCCTGTCTAGACTCTGCTCCCTGTGGGACTTCCCGCAGATTCCGTTGCTTTCTCTCTCTGGGCCTGTTTTCCTATTGCACA
ATGGGGATAATCACTCCTACCTGGAAAGGTTAACTGAGGTCACATGGATGAGGCGCCTGGAACCTAGTATGTATTCCCCTTATCTGA
GGCCATGACCTGGGGCTCTTGCTCTGTCCCTGGGAACCAGGCTTGTCTCTGAGTGGGCTCCAGGGGGGTACCAGGAACAGTCACAGG
AGCTCACTGAGTCCCAGCTTAAGCTGCTCAGACCCAGGGATATCTGTCTCTCCAGAAGCTCCCTGCCCTGCCTTCGCCGGCCCTCAT
GGCCCTGCCTCCGTGTGTACATGTGTATGTGTATTCCATGGGAAAGGCACAAAATAGCAGTCAGTCTCTCCATAGAAGAGCCTTGTT
GGTGGCCCAGTTTGACTCTCCCTGGGGCTGGACCCCTACAGCCTCCCTGGGAGGTGGTTGCAGCCCCCTTCCTCCAGCCAGTTCCAC
TTACTCCTTTCTTAGGCCACTTCCTCCCACCCTGCATGGGCTTGGTGGCTCGAGAATGTTGCCGTCCATACCCTGGGAGCTGTGCTG
AAAGGGCTGTGCGGCCCCCGACCACTGTGTGTGTCAGGGAGGGGGCACGCTCTCGTGGGGTGTCAGGCCAGGTGGCGGTGGGTAACT
GGCAGAAAGGCCCTCCTGGTGTGCTCTGGTGGCACCCTGTTGACCCAGTCTCAGAAGTTGTGTTCCGACCCTCACTGAACACCAGCT
GTGGGTCAGGCACGGGGCAGAGTAGTCCAAGTAGCTTGGTTTGCTGCCTGCCTGGGGACCTGACACTGTGGGATCTGGTCAGTGCTG
GGATGGCAAGCTCTGGGCACCTCAGGCCATGGGACACAGAGCAGGCTCCTACAGCAGCTTGGCTGGGTGGGACATGAGAGAGGGGCT
GGGCTGGGCACACTCAAAGGCAGGGAGGAGTCTGAGGGCCTGGCCTGTCAGGGTGGCCTAGGTGGTGGGTCCAAGCTGTGTGCTCTG
CACAGTGCTAGGCCTGTACTATAGTAGGTGCTCAAAAAATACTTGTTGAAAGAGTAAAGAAGCCGGGTGTGGTGGCTCATACCCGTA
ATCCCAACACTTTGGGAGGCCAAGGCAGGTGGATCGCCAGAGCTCAGGAGTTTGAGACCAGCCTGGCAATGTGGTGAAACCCTGTCT
TTACCAAAAATACAAAAAATTAGCCAGGCATGGTGGTGTGCACCTGTGGTCCCAGCTACTCGGGAGGCTGAGGTGGGAGGATTGCTT
GAGCCTAGGAGGTGGAGGCGGAGGTTACAGTGAGCTGAGATTGTGCCACTTGTACTCCAACCTGGGTGACAAAGTGAGACCCCCCTC
TCAAAAAAAAAAAAGACTAAAGAAAAGTGAGCCTGAGAGCTTAGGAGGAGCACATTTCAGAGGGGAACGGAGAGAGGAACATCAGGC
CCGTTGGTAGCTGAGGAGAGGTGCGGTTAGATCTGTGCTCCCCAAAGATCCTCTGCTGAACATAAGGGGCAACGCCTTGTCTCCTGT
GCTGTGTCCTGCGGGTGGAGGTGGATTGGAGGGAAGCGGAGGGCGAGGCCTGGTTGAGGGGCGGGGCCTGCCTGTCTGGTCCCCCGG
GCTGCCTTGGGCCAGCCTGGCCTAGTCTGTTGGGTGGGCGGGCAGGGTGCAGGCTCCTCTCCAGCCTCCAAGGGAGGGGAGTTGTTC
TGCCTCCTCGATAGCCCCAGGCCTTGGGCACAGCCCAGCCTCCCACGGCTCTTGGGCCCTCCTCCTTCCAGGCCGCCCGTGACCCAC
ACCTGGCTCTCCTCCCCGGCGTCTCCTCTCCGCTTCTTTGTTTGGAGCGGAGGCCCCGCCCCACCCCGCCCCCAGGCGCACTCGCCC
GGCCATTCCGGTTCAGCCGGTTCCAGCCCCCAGTTTCTGCCGCTGCAGGTCCCGGCAGGAGCTGGAGGGGCACTTTCTCCCTGGGTT
TCTCTTCCCTGGTGCAGCAGGGGCCGCGGTCCTCATCCTCCTGGTTCCTCAGTTCGGTCCTTCTTTCATTCTCCACCCCTGGGTGCC
AGGAACTGGGTCAGACACTGGGACAGGAATCCAGACAGGCATGCTATCTGCCCTGCCCAGGGTTATGTTCTAGGAGGGGAAGCAGCC
ATTAATCAAACACCAAAAATGTGGAAAAGTAATAATCTCACACGTGTGCATAATAAACTGTGAGTGAAAGTTATAAGCTCGGCAGGT
AGGTAATAAGCTAGGAGCAGTGCTGTGGGAGGCAAGGGAGTTACCCGGGAGTTTCAAACTAGGAACTGAGCTCATAGGTTGGGGGCA
GGGGGACTGGAGAAGGCAGTGATACTTAAATGGAGAGCAGAAGGATGAATGGAAGTTAGAGTGTATGGCGGAGGTTGGCAGAAGCAG
CAGCTTATGCAAAGGCCCTGTGGCTGCAGGGAACATGACGTTGCTCTTTAGAGGAGCCAAAGCTGGGGCTCTGGGGAGAGCAGCTGG
GTCAGACCCCGCGGCTTTGTCTGCCATAACAGGTGTTTTGAGAGTGATTCGGCAGGTCTTTGGAGGGTTTTGATAGGGCGGGGTGTC
GGGGGAGGCTGTCAGCTCACTCTGGACACTGAGTAGAGAACAGACGGGAGGCGTGGGGCAGGCCTGGAGGCAGGGGCTTCCGCGTGT
TAGGCCAGTGGAGTGCCAGTCAAGGGAAGGTGGTATCTGGACTAGGGTGTGGCAGCGCAGGTGGAGAACCCTGAGCTGCTTTGTGGA
GGGCTTCAAGTGTGGGGGAAGAGTGCACGGTATGGGGGTGGGGGACAGGAGACACCCCCAGTGGAGCCTGAGCAGGAGAGTCGTGTC
TGAGAGGGTCTGTCTGGAAGGCGCAACAGAGGCCAACTTTGCAGACAGCTGCAGTCGGGAGAGCCTGGAGCCTCCTTCAAAGGGCAT
TCAGGGGAAGGGCAAGGCACGCTGGGGGGTTCTGGACCTTCTGTGGTGTCTTCTTGTCTTTCTGGTCCCTACAGCCTCCCTGAGCTG
GCTGCCCGAGCCTGCCCTAGGCACTCTAAGAACATAGTCAGTCCCAAGGTCTCCCTCCAGGGAAGGCCGTAGGTGAGCTTAGGAGTG
AGAAGGCTGGATCAAAGCCTGGCTCCATGCCTGCATCCCTCTGACTTGCCAGTCATTTCACCCTCCGAGCCTCTATTTCCCCACCTC
TTAAATGGGGATAATAATACTACCTACCTTATGGGATTGCGGTAAGACTGATAATGCTGGTACGAGTGACAGCTTCCCTCATGGAAG
GCCCACCACGTAAAGCAGTTTACAGCCATCTCATTCCATCTATGACAGAATCCTGTGTCACTGTTTTGGAGATGGGAAAAAAGAGGC
TCAGAGATGGTGAGTGACTTGCATAATAATTACATAAAACCCCCAGAGCCCCTGGCCCCTGGAGTTCTCAAAAGTTCCTTCTCTGTT
GGTACCTGCAGCTGCCACTCCCTACCCCGCTCCCATAGACCCTCTCCTCCTTGGAGACTCTGCCCCATCTGCCGTCCCTTCTCTGCT
GGATCAACACCTTTTCCCTCTCTGCTGGCTCCCATCAGTATTTAAACATTGCCTTTCATATCTGTCTTCAAGAAAAAAAGAAAAAAA
AATTCACAAACCTCCCTTCCCGCCTCATCCTTTCCAGCTGCTGGCTGTATAGTCACCTGTACTTCCCTCTCTCCCTCATCGCCTCCC
AGTCATTCTTTGGCCTTCTTTGGTCTGGCTTTGGCCCCCACCCACCACTCCACTGACTCTGTTCTTGTCAAGGTCCCTGACAATCTT
GTGTGAACTGTTTTGTACCAGGTGTTTGACAGTCAAACATGCCTGAATTCAGGTCCCAGATGTGCCCCTCACTGGCATGTGATCTTG
GACAAGTGACTTGACCCCTCTGAGCCTGTAAACTGAGGATAATAGCAATGAAGGACTAAAGATAAAGAACCTGGTGCAGAGTGGGTG
CTTGGCAAAGGATTGTCATCATCGCACACGTTTCTGTGCCAGGGACCAGGCTGGGCCTGGGCTCCTGGGGACCAAACAGGTGGTCTG
AAAGGTCATTTCTCACAGCACTAGCCCTTTTTGGAGCTGTTCATTGGTCTGATTAATAGGAAATGGATCAGCTGTCAAGATTAACGA
GCTATTGCTACAAGATTGTAGCAAATGGGTTGGGCTTCTCTGGGTTCATGACCCTAGGTGGTTGAATTCTTAGGGATAGGGGCTGTG
GACTGGCCCTGGTATGTGTACTGAGGTGATGAGGGTGTGGCAGTGCCATGTCTGAGCCCCTACCTTTCTTCTCCTCCCTCTGCCTCC
CTGTGGACACCTTGAGGAGACTGTCAGAAGGCAATAACTAAGTCGGGGGGGAGGGATGGGAGAGGCAGATTTACAGGAAAGCATTCA
CCTGGGAAGATATCCAGAGAGACTTATGAACTGGACTGTCTAGGCCTTTGGGACTGCTGCTGGTATGTGGGGGCTGGGAGAGAGGGA
GGAGTCTCGGTTCCTGGCCGGAGCCCCGGGGTGGATGGTGGTGCCATCACTGAGATGGAGAGCAGGGGGAGGGACAACTCTCAGGGA
GAGCTGGAGCTCTTCCCAGCAGCTCTCCAGCACGCCTTTTTCCTGGAGCTTGGAATTGATGTGGGGCGGGCAACAGAAGGGTGCAGT
GGTAGTGTGAACTCCAGACTTGGAACACCTGGGTTCAGATCTTAGCTCTACCACTTACCACCTGTGTCATATGGGACAAATCCCTTA
ACCTCTCTGGGCCTCTAGAAACAGATACAGTTATAGCACTCACCTCATATGCTTAACAGAGTTAAAAAATGTTAAACTCTCTGAACA
GTGCCTGGCACATACTAAGCGCTACATAAAGGTGAGGTGTCCTTGTTTTCTTTGTAGGTCTTTCTCTCTGCCCCCATGACTGCCACC
TTCCTCACTGGCCATTCCTATAGTGACTGTGCTGTGGTGACTTGGTGTCTCCATCTCTTCCAGGCAAACCTGTCTTCATTAAAGTCC
CTGAGGACCAGACTGGGCTGTCAGGAGGGGTAGCCTCCTTCGTGTGCCAAGCTACAGGAGAACCCAAGCCGCGCATCACATGGATGA
AGAAGGGGAAGAAAGTCAGCTCCCAGCGCTTCGAGGTGCGTCTGTGGTGGGAAGGGGTCGGCAGGGCTCAGGGTCTGCCCACACTCT
CTCCTTTCAGTGTCCCTCCTCATGGACCTTTTGGAGGTGGGAGGACAACTGACCCTGAGCAGGCTCCTGTGTCCTGAGTAGGCTGTG
ACCCCATGTCTGTCCTCTGACAGGTCATTGAGTTTGATGATGGGGCAGGGTCAGTGCTTCGGATCCAGCCATTGCGGGTGCAGCGAG
ATGAAGCCATCTATGAGTGTACAGCTACTAACAGCCTGGGTGAGATCAACACTAGTGCCAAGCTCTCAGTGCTCGAAGGTACGTGCT
AGGGAGACGTGGCACGGTGGGCTGCCGGGCTGAGGCGTGGGAAGAGCCAGCCAGCCCTGATCCTGTCCTGGGCCCATGTGCATTTGG
CAGAAAGGAGGACTGGCCACCTTGGGGTCAGTGAAAGTCAGTGGTGGACAGGGATAGTCATTGGATCTGGCCTGGATTGTGCGGCTT
ATGCTGAGGCCAGCCATGTGGGGCATGATGCCTTTGTATTCTCCTGCTGAGCCGGGTCGTTGGTTGGGTGGGGTCTGGGGTCTGACT
TGAGGTGTGGAGCTGCAGCTGTGTATCCCTTGGGTTACGTGGTTATGGCTGTGCCTCTTTGGCAGTGAACCGGATTTCCATGTGGAG
CCTGGCCGTAGGTGTCAGGCAGGTGTGTTCCTTGTTGCCCCTGTGAGCTGAGGGCTGGGGCTCTGTCCGTGGATTTTAGTGTCTTCT
CTCACTTGGTGGCTTCTCCATTCATTCACAAACACTCCCTGGACCACCTTGAAGTCCTCTGAGCACCGAGGAGGAGGAAGCTGTGTC
TAAGCCAAGTCTTGAGGACAGGTGGGAGTTGGGGGTGGCAGTTGGCAGCTAGGCAGGTGCCCAGGCCCAGAAGCAAGAGAGGATGGA
GCTTTCAGAGAGCTCTGAGTAGTTCAATTTGGGTTTTCTGGAGGGCAGAGGGGGAGCTAGAGAGCACAGGAAGAAGGAGAAAGCAAT
TCAGCATGAGTCTGGAGAGGTTTGGAGGGCAGATTACACAGGATCTGGCTGAGAAATGAACACTCTCCTAGGGACATAGGAAGCCAC
AAACAAGGCGGGGGTGACATGATCAGACCCCAGCCACAACTGATTCATCAGTCTGAATGTGTTTATGTTTTCAAAATATAGCATCGA
TTGTTGCTTGCTTTTTTTTCTTTTCTTTCTTTCTTTCTTTTTTTTTTTTTGAGATGCAGTCTCACTCTTGTTGCCCAGGCTGGAGTG
CAATGGTGTGATCTCAGCTCACTGCAACCTCTGCCTCCCGGGTTCAAGCGATTCTCCTGCCCGGCCTCCCAAGTAGCTGGGATTACA
GGCATGCGCCACCATGCCTGGCTAATTTTGTATTATTAGTAGAGATGGAGTTTTACCATGTTGGTCAGGCTGGTCTCAAACTCCTGA
CCTCAGGTGATCCGCCTGCCTCAGCCTCCCAAACTGCTGGGATTACAGGTGTGAGCCACCGCACCCGGGCCGATTGTTGCTTTCTTT
TTAAGAATGTGATGTCGATACCTGTTCCTTTTTGAAAAATTGGAAAGTATAGAATAGCACAGAGGAAAAAATTAAAATGTCTCAGTT
TACCTCTAGTAATAACATTTGGTTACTTACTCGTGGCCCATTTTCTGTGCATACATATATATGTGTGTGTGTGAACAGAAATGGGAC
CACATACTGTCCGATGATTTGTAGACTGCTTTAAAAACAAACAAAAAAAAATATGGCTAACATCTTCCTTGCCACTAAATATTCTTC
TGTATCATTATTCTTTTTTTTTTTTTTTTTTTTTGAGACGGAGTCTAGCTCTGTCACCCAGCCTGGAGTCCCGTGGTGCCATCTTGG
CTCACTGCAGCCTCTGCCTCCTGGGTTCAAGCGATTCTCCTGGCTCAGCCTCCCGAGTAGCTGGGACTACAGGTGCGCACCACCACT
CGTGACTAATTTTTGTATTTTTAGTAAAGACGGGGTTTCACCATATTGAACAGGCTGGTCTGGAACTCCTGACCTCGTGATCCGCCC
ACCTTGGCCTCCCAAAGTGCTGGGATTACAGGCGTGAGCCACCACGTCCAGCCTGTATCATTATCCTTAATGGCTATGGGTAAGCTG
TCACATGCAAGTACCCTAATTTATTTAGCCATTCCCTTATTGTTGGACACATATGTTCTCAGTTTTTTCGTTTCTATAAATAAGGTG
CCATACACGTCGTTGCAGATGGATGTGTTCACCTTCCTGATTATTCCCTTATTCTAGGTTCATGGAACTGGAATTGCTGAGTCAAAG
GGCACATGCATTTTTAAGCCTTTTGATATTTCCAGAAGATTGTGTCAATTCATACTCCTGCCAAGCAGGGCAGAAGAGGGCCTCTTT
CCTGCACATCTTCCCCACTGTTGGGAAATATCTTCAAAAAAATATTTTTTGCCAAGTTAATAGGCAAAAAATGGCATCTCAATTTAA
TTTGCATTTCTTTGATTACAAGAACAGCTGCACATGTTTTCACATTGGCCATTTTTACGCTGTCGCTTTATCTGTTCACACACACAT
CTCCATTCAGTTACTCTTTTTGTTGTTGTTGTTGTTGTTTGGTTTTGGGGGGTTTTTTAGTTTTGTTTGAGACAGAGTCTCACTCTG
TTGCCAGGCTGGAGTGCAGTGGTGAGATCTCGGCTCACTGCAACCTCCACCTCCCGGGTTCAAGTGATTGTCCTGCCTCAGCCTCCC
AAGTAGCTGGGACTACTGGCAGGTGCCACCACGCCCAGCTAATGTTTGTATTTTTAGTAGAGACGGGGTTTCACCATGTTGGCCAGG
CTGGTCTCGAACTCCTGACCTCGCCCGCCTTGGCCTCCCAAAGTGCTGGGATTACAGGCCTGAGGCACTGCGTCCAGTGTCGGTTAC
TGTTTTGCATCTGTGTCTTGTATGAGTCCGTGGGCTCTACAAGCTTAGAGGAGCCATAGAGGACAGTGGTTAAGACTCTGGACCACT
CACTTCTCGTGTGTCTTTGGGTTAGTAACTATGCCTCTGACCCTCATTTGCTCCATCTGTAAATGGATATAATGCCTACCACCCAAG
CTGGTTTGGAAGATTGAATGCTGTGTATGAAGCGCTTCCCAGGAGTGTGGTGGTGGTTGCTGTGGGGCTGGAAGCTGTACATCTTGA
GGCCTCACCACGTGAGCTGAAGGTGGTTGGCCTCCGTGCTGCGTGGCATCATCCGGGTGATGCAGCTCCACTGCCTGCTCCCACGGG
GGGACATACCTGTGCAATAGGAACTCAGAGAACAGGCCCTGGGCCAGCATCACTGATTGCTGAGTGTTCTCAGGCTCCTCCCTGCCA
CTTCTGACCTGTTTCCCCACTCGTGACCTGGGGGCAAGTATTTCAATGCTGCCCAGTCCTCCTATTGGCAGCTGTCCTAGCAGGGAG
CCATCTGCCCTGCCCTGGCATTTGGGAGGTGGCTGAAGGACCAGAGGCCCCAGAGGGCTTCTTTTCAGGCTCTCAGATTGGGGAACA
GGGCCTCCTTGTTAGTATTGAAAGAGGAGTCCTTCAAAAGCGCCTAAGCCCCAGGTCCTGGGTCAGGAGCCCTCTTACCTCTGCCAC
TGCCCTGGGCAGCCTTACCTTTCAAAGGGTTGAGGAGGATGGAGGTGGTCTTTCAGGCCTGGGGTGAGGAGGACGGACTTCAGACTG
ACAGGAGGGGCTGGGTGTGGTGGCTCACGCCTGTAATCCCAGCACTTTGGGAGGCAGAGGCCTGCACATCACTTGAGGTCAGGAGTT
CGAGACCAGCCTGGCCAACATGGCAAAACCCCATCTCTACCAAAAAATACAAAATTAACTGGGCGTGGTGGCGCACGCCTGTAGTCC
CAGCTACTTGGGAGGCTGAGGCAGGAGAATTGCTTGAACCCAGGAGGAGGAGCTTGCAGTGAGCTGAGATTGCGCCACTGCACTCCA
GCCTGGGCGACCCAGGAGACTCCATCTCCCCCCCCCCCCCCGAGGACCTGCAGGAGGTGCCTCCGTTTGGTTTCTCTACCAGGGGCC
AGCAGTATATGTTTGCGCTTCTGGGGTGTGAGAAGTGTCTGATGAGGCTGGGGAGAGTGTGGCGCAAGTACCCAGACCCTGTCCATG
CTCCGGGGAAGGGTGTGGAGCTGTGGGCCTGAGTGAGCCCCTCCTGCTTCTGAAACATAGCACCAGGAAGACAAAATGCTGACTTAT
CCTGCTGGTCTGTATAACCTTGGCCAGCATTGGGCTTGACAGAAAGGCGTGCCGCAGGAGGGCCAGAGGGAAGCAACCCAGAGGGTG
AACACCCGCCCCCTGCCCCGCCTCACCCCACCCCTCCCCTCCCTCCTAGGAGGATCAAGAAAGCTGGGTGCCTGCAGGAACTGGGTG
AAAGGATGGTTGGACTTGGACTGGAGCTCTGGGGTGGGGCCGGCAGGAGCCAGGGCCATTCTTGGGAGGCCACCACAGGGTACGAGG
AGGGCTGGGGAGGGGATGGAGCTAGTTGGGGTCGGGGAAGTAAGAGTCCTTCCTGAGTTGCACACAGCTCTCCCTGTGAGCTGGTCT
CCTTGGAGAATGGCTGAGGGCTGTCTGGGGTGAACTGGCTCAGGTGAAGATCAGTGTGCCCCCAAAAAGGAGTCCAGGCCTGCAGTC
TGTTCTGTGCCCTCTGCCTTTGCCTCATGTCACACCAGGCCCAACTGCTGTGGCTCCAGGGCCACTCCCACCACAGCCCGTGGGAGT
GCCCCCCCAAATCCCCCACAGCCGTGCCCTTTGCACCTCGCATCTGAGCAGGATTGTTATTCCCAGTGTGGCCTCCCCTTGACACCC
CCGCCAGGACTGCATACGAGGTGGGGGTGCCCAGCACAAGCTGGCCGGGGTGAGCCTGTCCTGGCTGTGATGAGCGTGGGGCCCGCC
GCCCAGCGTTCCTGTCTGAGTGGTAATTGAAGCCATTAGCGCGCCAGCCTCTCCCTCGCCGGGTAATGGCAGGAAAAGCTCTTCTCG
CTCCGCACTCTTGAGGCGGCGGCTGAATCACTCCCCCTCCAACCCGCCCGCTGCCGCCACTGAGACAGGGAATCTGACATTTTCCCT
CACCAGGGAGGGGGAGCCCTGCGGGAGGGGAGGGAGGCAGGCCTGGATTCCTGGCCTTTCCCTCCAGGAGGTTGAGGGGCAGTGAAG
GTCTTGGAGCTCAGTCTGTAAGTCATGGATECACCTGGGGCCGCAGATTTCAGGCCTGGAAGGACTGGCCGGGAAAGCCCAGGAGGC
CGCCAGACATTCAGTGGTGTGGGGAGGGCACTGATGACTTTGGGTAGGTTCTGGGAGGGACAAGGAGGCGGGGAGAAGAAAGAAGCT
GTGGAGCAGAGATGAGCTGTTTTGGACTTCTCCTGGGGGGCTTAGCTCCAAGGGTTCTGAGTACAGGTAGCATTACTTGTCTTGGGG
TCATTGATAGGGCAGATAACGTGCACTGTTCGTGTGGTGTGGGTGCCCTGTGTGCCATGTTTAAGGCAGTGTTTGTGTGAAGTGTGT
GGTGCAGTGTGTGCAGCAGGGGTGCTGCCTTGCAAGCTGTGCGCACAGAACGGGGTGTGCAGCGGTCAGCGCACACGTTACCATCAG
GCAGTGGGGAAGGGGCAGTGTGTGCAGGAGGCACTGCATGCTGTATGGGCTGTGTATGCAGCGCGCAGTGTTTGTGTGCACGTGTGT
GCGTCAGGCAGTGCGTGGTGTGGTGGTAAGCAGGTAGTGAGTACCTTATGGGCTATGTATACAGCAAGGCATATGCATCAGGCAGTG
TGTGGGCAGTAGCCAGTGTGTGTGTGTGTTATCAGGCAGTATATGTGTGCGGTGTGCCGTGCGGCAGGCTACCTGTGTTTATATCGG
GTAGTGTGTGTGTGTTTATGTGCAGCAAGGGTGTGCAGTAGGTGTGCAGTAGACTGTGTGCAGCAGGCGTGTGTGTGTGTACAGCAG
GTGGTGTGCAGCAGGCAGTGTGTGTATGTATCAGGCGGGGTGTGGTGTGTGTTTATGTGCAGCATGCGGCGAGTACTTTGTGTGGTG
TACAGCAGGTGGTGTGCGGCAGGCTGTGTGTATGTATCAGGCTCTGTGTGTGTGTGTGTGTGTTTGTACAGCAGGTGGTGTGCAGCA
GGCAGTGTGTTTATCAGGCAGTGTGTGGTGTGTGTGTGCATCTTTATGTGCAGCGAGTAGTTTGTGGGGTGTGCAGCAGGTGGTGTG
TAGCAGGCTATGTATGTATCAGGCTGTGTGTGTGTGTGCGCGCGTGTGTACAGCAGGTGGTGTGTAGCAAGCTGTGTGTATGTGTCA
GGCAGTGTGTTTATGTGTGTGTGTCCAGCAGGTGGTGTGTAGCAGGCTGTGTATGTATCAGGCAGTGTGTGTGTGCGCAGCGGGTAG
TTTGCAGGGGTGTACAGCAAGCAGTGTGCATGGTATGTACAGCAGATGGTGTGCAGCAGGCAGTGTGTGTGTGTGTGTGTGTACAAT
AGGTGGCATGCAGTGGGCAGTGCATTTGTGTGCATGCAGCAGGCTGTGGTGTGTACAGCAGGTGGTGTGCAGCAGACTGTGTGTTTG
TATGCATCAGGCCGTGTGTGTGTGCAGCAGGCAGTGTGTGTGTGTGTACATGCGCACACGCAACACGCACTATATTTGTGCAGCAGG
CCATGTGTGTGGTGCATACAGCAGGTGGTGTGCAGCAGGCTGTGTGCTTGTACATGCAGCACATAGTGTATGTGTGTTGATGTCAGA
CAATGCATGTGCATGTGTGTGGAGTGTACAGCAGGTGATGTGCAGCAGGCTGTGTGTGTAGGTGTGCCTCGGGAGTGTGTGGGGGTG
GGAGTGGACAGCAGGTGGTGTGTGCCTTACAACCTGTGTGGGCAGCAGCAGGAGTGGGCAGCAAGCAGCCCGAGCTGAAGTGTGGTG
GGGTCAGTCTGGCACTGGCAGGAGGTCGGAGATGCTGTGTCTGGGCTCATTGTCTCTGGGACCCCGCAGGATCCCTGTTTGGAGCTG
TTGGTCCGCCAGGAAGTTCGACAGAGTTGGATGTGGCCAGAATGGTCTGGCTGAGCTGTTCTCAGCACCTGTTGTGGTCGACCTCAG
CAGCTCCTCTGTAGCCAGGCCAGGAGGTGGGGTGCACCAGATAGGGATCCTGCCCAGACCTGCCTGCAGGAGGTCTTGGGACCCTTT
CCTCTCCCTCCCCTACCCCACCAAGCCCACAGTCCCTTCTGTCTGTTCCCACCTGGCCTCAGTGTCCAGCCAGCACCATACCACCTA
TGCCATGCGGGCCAGGGAATCCAGGTGTCTCGATGGAGTGCCTGGTGTTGCCAGCCTTGGAGCAGGCTCTGGGGAAGGGCTGTCCGG
CACATGAAGTAGTGACCAGGGTGGGGGCTAATCAGTGCTGTACTGGGCTTGGACCTCTGGGTTCTGCAGGAGCTCTGACCCTGGCTG
GCTTTGGGGCTCGTGGGATGACGAAGGACCTCTTGATATACCCCCTGACCACTCCACAACACAGTCCTTTTGGAAATAGCCCTAGAG
ACAGAGGGTCAATGATAGACATATGACCCTGTCCTGAGGTATCCTGGGCTGGGCTGGAACTGGAGCAGAGCTGGTGAGGGAGCTTAC
ATTTGCAAGATGGGTACATGTGTGTTTTCTCAAGGAGTCATCTCTCCCTGCTCCACCTCCTTCCCTGCTCCAAACCTGTATATTCCT
TCCACCCATGGTGGAATGACTTGGCCCAGGCCTGACTTTGATGGCTGGGGAGTTGGGTAAAGGGCTATTGCAGGCACAAGATGGCTT
CCCAAGTGGCAAGATGTGATCCCTTTCTATTGTCTACATGTGTGTCTGGGGTAACAGTCTGCTTTGGCAGAAGCTGGGGCTGTCAAC
TACCTGATTTGCAGCAAGAGGATGTAGAAGTGGCCCCAGAACCTGGAACCCAAGCCAGCTTCTACCTCTCCCTTAGCAAACTGAGGC
ATGACCTGTATACCCCTGGTCCAGCTGCTGTAGTCCTCTGTGACCTGTCCTTACACCCCCATGACTGCAGTAGATTTCAGATCCACG
CAGACATGAGGTTCTACAATCATTTCCTGGAGGTTCTAGGCCTCAGGGATGGGGATGGGAGAGGTATTAAGTAAGTTCTCCTGTTTT
TATTAGCTGTGATCTGCCTTGAGGCGCCCAAGTGGATTCCCTTCCGAATGGGTGCTCCTGTTTAGTAGGTGGTACTCCTGCCTCTTC
TGTTAGCCATGGACACTGGGCACTTCCCCTGTGCCCAGCCTGCCCCCCTGCCCCTATACTCACAGGTACTTGTAAGGCCCACTGTAC
GTGTTCCCTAGCCTCATGACCACGGTGGAGCCAATGGCTCTGGGAACAGGCTCACCAGGATGGCGGTTTAAATGCCCAAATGCCCTG
TGCATGCACATTCTCCATCCTTGGTAAGGTGCTCCCTCCTACTGGTTTGCTTGCTGGGGAGAGAGCACTTGTTTTTTTTTTTGAGAT
GGTGTCTGACTCTGTGGCCCAGACTGGAGTGCGGTGGCACAATCTCACCTCACTGCAACTTCCGCCTCCCGGGTTCAGATGATTCTC
CTGCCTCAGCCTTCCGAGCAGCTGGGACTACAGGCACGCGCCACCACACCTGACTAATTTTTTGTATTTTTAGTAGAGATGGGGTTT
CACCGTGTTAGCCAGGATGGTCTTGACCTCCTGACCTCATGATCCACCCGCCTCAGCCTCCCAAAGTGCTGGGATTATAGGCGTGAG
CCACCGCGCCTGGCCCTGAGAGGACTTTTTTGAAACGGAGTCTCACTCTGCCACCCAGGCTGGAGTGCAGTGGCGTGATCTCGGCTC
ACTGCAACCTCTGCCTCCTGGGTTGAAGCAATTCTCATGTCTCAGCCTCCCGAATAGCTTGGGATTACAGGCATGGACCACCACGCC
CGGCTAATTTTTTGTATTTTTAGTAGAGATGGGGTTTCACGAAACATGTTGGCCAGGCTGGTCTTGAACTCCTGACCTCAAGTGATT
TGCCCGTTTTGGCCTCCCAGAGTGCTGGGATTACAGGCATGAGCCACTGCTCCTGGCCAAATTTTAATCAGAGCAATGACGTGATCA
AACTTAAGCTTCAGAAAGGTCCGTGTGGGTGCATAGTGGGGGCTGGAAGGAAACCAATGACAGGTTGTTGCAGTCCAGGTGAGTGAT
GGCATTGGCGCAAATCTGGGTATGGGTAGGGGAAGATAGGGGAGCAGATCACTAGAAGGTTAGAAAGAGGCCGGGCACGGTGGATCA
TGCCTATAATCCCAGCGCTTTGGGAGGCCAAGGTGGGCAGATCACTTGAGGTCGGGAGTTCGAGACCAGCCTGGCCAACATAGTGAA
ACCCCGTCTCTACTAAAACTGTAAAAATTAGCTGGGTGTGGTGGCAGGAGCCTGTAATCCCAGCTACTCTGGAGGCTGAGGCATAAG
AATTGCTTGAACCCGGGAGGCAGAGGTTGCAGTGATATGAGATGGCGCCACTGCACTTCAGCCTAGGCGACCAAGCGAGACTCCATC
TCAAAAAAAAAAAAAAAAATAAGATTAGAAAGAGTAGAGGCCTGCCTGGCTACCTGTAGGATTGGGGAGGTGCAAGCCTCCAGGTTC
CTGGCTCTGGGGCTGGGTAGGTGGGGGTGCCATTTCTAAGATTCATGTACTAGACGAGGACCAGGTTTGAGGAGGCGAGTTGGTGAG
TCCAGTTAGAAATCTGTTGAGCCAGGTGTGCGCAGGCATCCAGGGAGGTGTCAAAGAGGCCTCGTACACATTTGCCTGGCACTCCGG
GGGACATCTGAAGTTGGAATCTTACAGGCTGAGAAATAGCAAATCTCAGGAGGCCAGAGCCAGGACAGAAGGAGCCCCAGGAACCGA
CTCTCCATGTTGGGAGGAGGTTCCTGAGAGACTGGAGGGAAACTGGGAGTGTGTGAGGTCAAGAAATGCCAAGAGAAAAAAGTGTGC
GAAGGACGAGGAGTGGTCAGCAGGGCGGGGTGCTGCCGAGAGGCCCGGCATGTGTCCTTAACAGTTAGTTCCGTGGAGCCATTGTTG
GCTTTGGTGTTGGGAGACAGCAGGGCTGAGAAGTGAGAGGGAGGCTATCAGTGTCTGTAGTGACTGGAAGGGCCCGAACCTAGGGAG
TGAATGGATGACAAGAGTCTCCCTGGAGTAGGACTGAGGTTGGGGAGCCACTGAAGGAGAGTCCAGTGTGGGCCCGAGTCTGGAACT
CCAGTGTGGGTCGGGTGTATGCGCAGGGCATGGCCCCTGGGTTCTTGTGGTGGGGAGGCCTCAACTGGGTTGAGTAGGGCGATGTCG
GGTGCCAGGGAAACCCGAGGAAGGAGGGGATGACTGGGAGCGGGACGCCGTGCCTGCCAACAGGCCCCAAACAGCTCAGATTGAAAA
ACAAAACAGGCTTTTAAGATGCCAAGTTTGATGAAATCTGAGTGCTGGAAGAGGTGGGAGTTTGCTCTTGGAAAAACAGCTGAAAAT
CGAGACTCAAAGCTGCGAAGGGAGATGGGCCCAAGGCTCCCCAGGCCCCCCTTACTGCCTGGAAGCCTGGGGGTGGGGCTGGGCTCT
CTGGGAGAGTGTGGGAGCGTGCAGATCTGGCAGCCTTCGACTTTTGGAAGGCGTCTGGCTCTGGCCCTGCTGAGGATGGAGCTGCTG
GAGGTGGCCTTGGCTTCTGTGGAGCGGCTGAAGGGCAGGGGGAGCCAGCAGCCCTGCCACCTACGAGGTTCCTTCATGTGTCTCGTC
CCTGCATGTGTCTCCAGGCGCACGTGTTTTGGCATAGGTGTGTCTGGGTGTGCATGTCTCCACATGTGTGTTTGCGTGTCTCTGTGT
GCACCGCCTCTGGATCTGCTTGCATGCGCCTGTTTCCACAGGTGCTAGTCACTGCAGGTGCCCCTCCACATGCACATGTAGGTTTCT
ATTTTTACACCTGTCTGTTTCTCCATGTATATTTCTGTGGGTTCCTGGCTGTGCATGTTTCTATGGTGTCTGTGTGTTGAGAGGCAG
GGTGAGGCCAGGCCCAACACTCAAGCTTAGGGGAGGCGGTCAGGCTCACAGACGGTGCCACCCCAGGGGGCCTGTGTCTGTGTGTGC
GGGTGCGTGCCTGCGTTTGCACGGAGACGCCACGAAGCTGGGTAAACATGGGTGAAAAGGTCATACTGACAGACGGTGCTGCCTGCC
CCAGGACCCTGCAAGGCGTCTCCATCTGTCAGCTTCCCTGGTGCAGCCCCCTCCCTCTGGCCCAGAGACTCACCCTCAGGCGTCCAA
GGCTGAGCTGGACAAAGAGACCTGTGTGTACTTTGTAGGGGGCCTCAGCAGCCTCCACCCCCATCTTAGGCTCCTCTGTCAGGACCC
CAACACATGCCCCAGCTCCCACCAGACTCGCCTTGGTACTGTCATCCCACCACCTTCCCCCACAACAGCCTTTACAAAGGCAGTTTT
CCCCTCCTCCCTGGAAAGCTTTCTGCCTCCCATGCTCATGTGTTTCCTGTTCTTGAATCTCCCTCCTCCAGGAAGCCACCAAGATAG
CAAGTGAGCTGTGGAGTCAAACCGATCAGCTCCAACACTGCTGTGGGAGGTTAGCCAAGCGCTCATCCACTTCTGAACCTTGGATTC
CCACCATCGACCCCCGACCCTCCCCTCGCTAGGGCTTGTCATCGTCTTCTGCCCATGGGGCAACCAAACCTCTCCACGGAAGGGGAC
AGGTCTCCTTGCTGCAGTGGGTAAAGGCCAGCGCAGTTAGGTGCAGGAGGCATTCACACACACGTGCACACTCCCCACCTTGCACAC
ATATCTGCGTGAGCCGGGGAGACCCTAGGGAATGTGTGTGCATGTTCTCTATGCATGCGGGTAGAATCCGCAAACGGTGTGGAGACT
CGGGCTCTTGGGTACCTCTGAAGGCCCCTGAAGTCCCCATGGGCTTCTCCTTCCGTCCAGGGCACCCTCTTATCAGGCCATGGCCCT
GAGACGCGTAGTGCAGACGCCCCCGGCGCTGAGGCTGAGGAGGCAGATGGCCCCTCCCCGCACTGTGCAGGGCACCCGGTTGGGGGT
GGAGGGGAGGGCCGCGTCGGTGAAGCGGGAAAGCCTAGTGGGAGGATTCCCTGGAGCTGAGGAGCCGGGGCCTGGGAAGGGGCGCAG
AGGCTCCACCCAGGCGGGGGCGGGAAGGGCGGTGCCAGGGCGGACAGCGGACGCGCGCGCCTGCACGGACTCGGGCACACGCAGCCC
TTCCGCGGCAGCGCCCGCCGCTCCACCGTCGCCATGGCTACCGGCTGGCCTGGAGCGGGGAGGGGCCCTTCCTCCCCTTCGGCGCCA
ACAGCAGGCGATTTGAGGGGACTCAGTGTGACTGGTGCATCCCGGGGTTGGAAAATGGGTGGGTGCTTGCGACTGTCCACGTGTGGG
GGACCCTGGGGTTCGCTTTGCGGTAGATGCAAACGCCGCGGCGCGTGTGCGCGGCTCTGCAGTGGAGCCTGAGCCGTGCCGGCCGAG
GCGTGGTGTGGGGGAGGCTGCCGGCCCTCTCGCGCGCGGGGTGTTCACGCCTAGAGCGCTGGGGCTGGGGGCCTACCACCCGGTCTC
CTCCCAGCCCCACCTCCGATTTAGCTGTGTGACCTTGGGCAGGTGTCCAGATGTCTAGATCTCTCTCAGCCCCTGGTTGCCCATGTA
CCTCATAAGGGTTTGGTAAAGATTTAAGTCATTTTGTAAAGCATTTTAACATAGTACCTGGAACCTAGTAAATGCTTCATAAATCTT
ACTGTCCCTCATACCTGGGTCTCAGTTTTCCCAGTTGTTGATGGGTTTGGAGTGATCATGTGACATCATCTGAGGAGTTGCCCAGGT
CCTCAGCCTGAGTGTTGAGGCTGCGGTAGACCCAGCTTCCGCGGGTGCCCGTGGGGGAAGGTGGTAAGTGTGCCAGTCTGGCTGATA
GATCAGTTTACACCAGGATGCCCAGTGCTCAGCCAGGCCAGGCGCATGGTGGGCGTCAGGAAAGGGCTGCTGTACTTGGCTGAGTTG
AATGTTCAGAGGCGCCTGCATGGGAGAGAAGGAAGAGGCAGCAGCAAGTCGCTCCTGAGGGGCTGGAGCCCTCCTGTAAGACCCACT
TCCCTTCCCGGGTGGCAGAGTGGCAGACTTCCGAGTGCTGCCTAGAAGCCTAGTTGGCACAGGGGACTGGCTTTTGGGTCCCGCTGT
TTTATGGACAGCTCTCCACACATTCTGGTTTTAGGCTCTGGCGGCAGTGCCTGAGGGATGATCTGAGCCAAGGACAGAGCCACTGAG
GGCGTGATAATTGAGGGAGGAAAATTAATTGTCCTTAATTTGGCGTAAATCCCAAAGACCTTCCTCGTGTAAGGAATTCAGAGTAGA
TTCCGAGACACAGGGCTGCACACATTTGTACTTCCCTTCCCTTCCCTATCTGCGGGTGGAGATGAAGGCCACTTGACTCCTGGGCCC
TGACTCTGGCAGGCCATGGCCACGTCTTCCCCATGAGCTGGGCAGGTAGGAATGAGGTCTTGAAAGAGTTAGACTGGTGCTCTGGAG
GGCACCCAGGATGGCCCTAGCAGCCCCGAGTGTCCCCAGGTGTTGGGGAGGTGAGCCCTGCACCTCTGGTCCCCCTCAGGCCTTCCT
ATGGAAGCAAGCAGCAGCTGGGCCAAAGGAGGCTGATCCCCTGCCTGGTGCATCTCAGTCCTCTTCATTTCCGTCCTCCTTCCCTTC
TCTTGCCACTGTTGAACATTTTACTTTTAAAAATCTGAAAGGGCACTGTGGGATCATATTTACAGCCAAGGAGACACTGGGTTTATA
TCCAGAACTTCTAGGGCTGCAGGTGGGGAAACGTACAGCCAGGTCCCAGGAAAGGCTGGGGCGGCAAGGCCGTGCTGGGAATCCTAT
TGCTCTCTAGCCTGAGACCTCTGCTCCTCATGGGCCACAGTCCCTTTGGGATGTCCCTGGCAGCTAGAGCCTTGGGGAGCATCCCCT
GCGACTGGCAGCAGATAGATAGGTATCTTGCTCCTGCCTGTTGGGGCTCGTCCAACTCCCTCTTCTACCCGCCCCCCAGTCGCTCTC
CCTGGGTCTCCAAGAGGCTCCAGGGACGGCTAGTTTCTGCCAGCCTTTACCTTCTTCATGTCTGAGGATGCCATGTGCCTTTACTCT
GGCATAGAAGCCTGACTTCCCTTGGCACATGTTCCCATACACCCATCTTGTGCTGGGCTTGTGGAAAGGAGGTAGAGTGGTGCTGGT
CTCCCCCACCATGAGCCCAGCTCCCCGCCTTCCCCAGGAGACAGACAAAGAACACACATTCCCCTTGCCCCACATTGGGTGTGTCTG
GCATCCACACTGGGAGAGACACTCTGCTGAGGCCTTGAAAATTGGTGGTTTGCGATGGGGCCTGGTGGCTCACAGCTGTAATCCCAG
CACTTTGGGAGGCCGAGGCGGGTAGATCACCAGAGGTCAGAAGTTCGAGCCCAGCCTGGCCAACATGGTGAAACCCCGTCTCTACTA
AAAATACAAAATTTGCCGGGCGTGGTGGTGGGCGCTTGTAATCCCAGCTACTCGGGAGGCTGAGACAGGAGAATTGCTTGAACCCAG
GAGGCGGAGGTTGCGGTGAGCTGAGATCGCACCACTGCACTCCAGCCTGGGCAGCAGAGTGAGACTCCATCTCAAAAAAAAAAAAAA
AGAAAAAGAAAATTGGTGGTTTGGTCCTAGTGGGAAGGGCCTCTCACCAGCCTAGAGTGGAAAAGGGAGTTCCCGACTCTAGTCTCA
ATACCTGTCTGCCCCAGTGGCTCAGCCCTTACTAGTTACACCAGCTAATATTCACTGGGAGTGAATTCCGCATCAGATGCTCCGCAA
CACCTTGCATGCATGATCAGATACAGACCTCACAGTAGCCTTACCATCAAGGTGGGCACTTGAACCCTGTGTACAGATAAAGGAGAC
AAAGAATGAGTAACATGCCAGGCCCTAAAGCTAGTTCGTGGTGAACGTGGGAGTCCCATTTGATCTATACTAGTCCTGAGCCTGTCC
TGGACTTGTGCTTCCAAGGGGTGGAGAGTAGATAGCCTTGCCCTGCAGCCCCTCGGGGCTGATATGGGAGCCCATGTACAGTGGGAG
TGGGTTTGCTGCTGACATCTGTTCCTCTTACTCTATGCTAGTGACTCCTCTGTGTGCCGCCAAACCCCTAGCAAGCTGGGAGGGGCA
GCCAGGAGGGAGTTTTTTCTCCCCCTACCAACTTTTTGTGTCTTTAGAGCTTTTTTATCTCCTTTGCCTCCACACACTCAGGCATGG
TCTGCAGCCCTGACCGTGACTCCCAGGGTCAGGACTAAGTGAGGGAGAAAGCTCAAGGTCAAGGCTGCAGTAACGAATAGGTCAAGG
TCAGGATCGGAGTTAGAAGGGGATCATTGGTAGGGCTGGGGGTGCCCCGGGTCAGGGCTAGAGACCAGGCGGTGACCTGGGGCTCTG
CCATGTGATAGAGCTGAAGGCTGGATGAAGGGAACACTGTGTGTGCGGACAGGGGAGAGGGGGCTGGACAGCACAGAGGCCTTCAGG
CTGAGCTGTGGCTGTTGGATGCTGCGCGAGCTCCCTGCTAGCCCCCCCCCCCACCCCCTGCAGCCCCAGCATTCATGCAGTGCTTTC
TGCTGTGACCAGCAGAGCATTGATTCTCGTTCTTCTCAGGGCCTGGAGAGATAAGTGCTGACGCCTTCAGTCTGAGGCGTTGCCTCT
CAGACCTGGAAACTCCCTGACAGGGCAGGGGTGGGCCCCACTGCAGCCTCTGCCCTGCCAAAGAGACTTGGGACCCTGGTTCCTCAA
ATCGGGGTATGCTTCATGCTTAGAAGTCAAGGAAAGGGGAGGGGAGTCTTGAGGGCCCTGGCCAACCTGCAGTTGGGGAGGTTACCC
CCAGAGGGGTCATAGGGGGCAGGCAGAGCCAGCCCTAATACACACATTGCTGTTTGTCTGCAGAGGAACAGCTGCCCCCTGGGTTCC
CTTCCATCGACATGGGGCCTCAGCTGAAGGTGGTGGAGAAGGCACGCACAGCCACCATGCTATGTGCCGCAGGCGGAAATCCAGACC
CTGAGATTTCTTGGTTCAAGGACTTCCTTCCTGTAGACCCTGCCACGAGCAACGGCCGCATCAAGCAGCTGCGTTCAGGTGAGCAGA
GGGCAGGGGTCAAGGGGCCATGCAGACCTCAGAACAAGCGTCTTGTCAGATCCCAGCACAGCCTACTCCCTTGGGCCTGGGCACCTC
CAGGGCTGAGCGGAGGGTACCTGGTGGGGTGGGCTGGGTCTTACTGCAGGTGTGCCTGGCTCAGGGAAGAGAGCTCGTGGTTGGCTG
TGCCGTTACCTTCTTCGGATTGTCAGACTCCAGACTTTGGGCCAGTTCTGCCCCTCCCAGCACATGTGATGTGCCAGTGTGGTGGAC
TCTTCAAGGGTGCTCTATGGATGTTCACCCTCCTCCTTCCCTGTAGCCTGGCCTGAGACAGGGCCTGGATGATGCTTCTCTTTGCTT
CCTCAGATGGCAGGGCTTAGCTGGGAAAAGAGGCTAAAGGTGCCTGATTCATCAGGCTTCAAAAGGCTGGATCTCAGGGGCCTGGAA
CTAAGGGGACTTGCTGTTGTCCCTCGACCACCAGAGCCACCTGTCTCCTCTGGATGTCTCCGTCGAGCCAGCTCGGAGCCCTGGGAG
CAAGGATGCCATCGTGCAGGAGGGAGGTGTCACCCCATTGATCGATCTGCCTGTGAGGCTCCTGCCAGGATAATTGATTCAGTTTTT
GTGGGAACAGAGCAGGCGGGAAAAGAGGCTCAGAATCAGCTTGGCTGCATTCTGCATCTGCTGCCAGCACGGCCTGGACCAATAGTC
TTTGCTTCAGAAGCCCTCCTGCTAGCTATGGGATGGTTGGCCTCGGGCAGGATGGCCAGTGCCGGCCAGAGCCCCTTCTGCCTGTCA
GTTGTGATGTCAATGATGAAAAGGAGGACATGACTCTTGCCCCTTTCAGGGGCCTGCAGGCTTCAGAGGTGCCCTCCCACTCCCTGG
GATGCCAGCCCCTCCCCATCAATTCCCACCAGCCTCACAGCCCCTTGGTGCCCAGCAGGAGGAGGGAGAGACAAGCTGCCCAGCAGG
GCAAGATTCTGGCCCCAGCCACGGCCGCCTGAGACAGCCCACGAAGTGTTAGCTCATTTAATTTAATTAAAACTCAACAAGATGGAG
GCAGCTGTAGCGCAGTTAATTAAAACAGCCATAATCAAGGCAGCAAACGGCCGGCAGTGTTTGTGGCCGCTGCCCAGCGCAGCACAG
CGGCCAGCACGGTTCGGCTCCTCTGCATTTTCTCATAGTTCCTCCAGCCAGGCTCCCCAAGCAGCCAGACGCTCCTCCCTGCTGGCC
TGGGCCCCTCCACAGAACCACATGGACTTGTCTGGCAGCAGCTCTGGGAAGGCTCGCTCACACATTGGTTCATCTAGTATTTATATA
GTGCTTGGGGTGCCCGGCCCTGGGCCACCCCTTCCTTGCCTATCACTCCACTGGATGCCACTCAGGCCCCATCCTCCTTGTCCTCTC
GGCAGCAGCTAACATTCCTCCCTCCTTCCCAACCACACTCCCATTTTTCCCTAGCCTTTCAGGGCTCTGTCTCTTGCGACTTCCCTC
TTCCTAGTGCCCCTTCCCTGCAGCAGCATCTTCCCTTCCACCAAGCCCTGCCTTTGCCCTATGGACACAGCCTTGCCCTGGACGAGC
TCACCCCCTCCTAAAGCTCCAACTGCCATCTCCTCACCTGCAACTCTAGCTTTAGTCTTTCCAGCCCAAACCTCTTACCTGAGCTCT
AGATCCAAAATTCGACCTGCCTTCTGGTCACCCTGACCAGTGGTGATCAGTGATGAGTGATGACCTTCTTTTCAGACAGCTGTACCC
TCTTTCCATAAGTGGCACCCTCCAACCTGGAACATGGGTCTCAAGTTGGTTTGAGAGCTCTCAAATGTACCACCCTCGTGGCTCTAA
AATTGGCCCCCTTCTCCCCTCCTGCTGCGTCAGTCCTATCCCAGGCACCAGACACTGCTTTCCCTGCCTCCGGGCCCTCCCGAGAAT
CCATCATCCTGTAGGTCAGGTTTGCTGCTCATACCTTCCTGTGCCTCAGTGGTGTCTCCTTGCCTACCTGGTCAAGTGACGCTCCCA
AGCAAGGCTTAGAGGGCCCTTCTTGGTCTTCCCCTGCCCGTGTCTCATCGGGTCCTGGCTACACCACTTACCAGCTCTCTGGCATTG
GTTAACTTTTTCGTGTGTCAGTTTTCTCATGTTTGAAAGGAGTTACAGTAAGCAGTGAGTGAGTCAATGGCAGTCCCCCCCTTGAGT
TGATGGCCTGGCCTCTGGTAAGGGCTTCATGGAGCTCTTTCTACTTTCTGTACCCTTGACCCTCCTAACACTGAGCTGTGCTGACCC
GTCCTTGGTCCTTGCCCACCTCCGGCCCTCTGCTCACCTTCCGGCTGCTCCACTTGAAAACCTCCACCCTGCTCAGCCTCTGGCTGC
TGCCTCAGGCCTCACCTTGCTTTACATTGTCACCATCTGGCTCCAGTTCTGCTCAGCTAGGCCTTGCAGGGAGGGCCTGGGTCTGAT
CACACTTGGTGAGGTCAGCTGTAGGACAGGTCTTCTCTGAGCCCTTGTCAAGTGAATGATTTCATGAACTTGACCTTTGGCACTTGT
CCCTGTAGGCTAATATCTGCTCTAATGTTCACTCCTCCTTCTGCTTTCCAGGTCCAAGGAAGACTTCCTTATTTGTCTTCCTGCCAC
CTGGAAGTTGTGACCTCAGGGTCGTGGGCCCAGGGTCCAGCTCCTGGGATGGAGCCAGATGGCACCTAAGGGGCCTCAACCATCCCA
CCTCTGCAGTAATAACTGGGCTCTCTCCCCTCCCTGCCTGACCTGGCCTGGGACCGTTGGCCTCAGTTGTTGCTGGCCTTATCCCAT
TACCATTTAGAAGGGTGCTAAGGCTATTCCGTGCACATTTTTCAGGGACAGCCCTTCATGGAGTGGACTCAGGCCCCTGAGCACTCA
GCTGTTTACCGGGACCTTTACGGTTTACGCATCACCGACAGTTTACAACAGAGCTTTCCCCGCTTTTGTTGCAGCTGATTCTCTTGC
AGCCCTGTGAAGTAGGACAGGCTGTGATTACCATGCCCCCTTCACAGCTGAGTTAAGGGAGTCACTTGGGGTCACACATCGAGACTC
GGCCTAGGAGCTCCCCTGTGAGATCACCTCAGGACCTAGTATCACAATAGCAAACCTGGGGACCTGAGGAGCAGCTGGACCCTTCTG
GGGCTTCAGAGCTGCACATTCCCAGCTTCTCCAGACCCCAGGCCCCCACTGACCAGTACCCAGAAGTCCTCCACCATCTGCAACCTG
AGCCACAGCACATCTAACCAGGGCATGACCCCCCAAGTAGGAGCTGGACAGGAGGTAGCTGACGGCATGCGCTGCCCAGATGTGAGC
TCTGCTCAGCAGGCCTTTTCTTCTCTGTAGTGATGTGACATGCTGCCAAAACCACCTCCTGGAGAATTGGAACTTGAGACCGGGGTA
GGCCCAGGAGGAACAGGAACAAGCTTATAGAGTGGAAATGGAGTTGTGAGCAGGGGCTCAGAGCCCCTGCTGGGTCCTGAGAGGAGC
TGTTGGCCTGCAGGCTGTGCCGAGCCTGGACAGGGCTTGAGGAGATTCCCGCACTCCTGCTGTGGCCTGAACATATGAGCTGCCATC
CTTTGTCGTAGAGGACAGCCTAACTCACTAAGTCCATGTGCTCATCCAGAGAGCAGTCTCTTCCCCACCCCCAGCACCCTGAGGCGA
AACCTGGGGGTCTTAAGAAGAGATGCAGCATCTGGCTGCAGGAGGAGGCCCGTGGGTGGGACGCAGAGGGCTTGCAGCCCCTCACCC
TGCTGGCTGGCCCCAGCTCTGGCTGGAAGAGCCTGTCCCCACCCCACTCTGCTCTGCCATCTGCGGGGCCTGCCAGGAAGGCACACT
GCCAGTGCATGCTCACAATTTCCCTTTGGCCCAGAGCTCCCTGGCACCTCTTGGACACGAATACACCCCTAAGGATGCTGACTTCTG
GGCCCCTTCAGTCCCCCACACCCATCTTGTGAAATGGAAAAGTCAGATTCTCTGTTTGGTGGGGAAATTACTGTTAGATTCTTTCAG
AATAGGTTAGGTTCTGGAAGAGCTGAGGCCAGGAGCGAGGGATGCCAGCCCTGGACCATATCCACTGCTCCCACCCCCACCAAGTCC
TGGCGTGGATGACAGGAGATCAGCAATGTCAACTTTTTGGTCTCAGGACCTCTACTTGTAAATATCAGAGAACCTCAAAGAGGGTTT
GCTTGTGTGGGTTCTTTTTTTTTTTTTTTTTTTTTTTTTTTTTTTTTTTTGAGACAGAGTTTCGCTCTTGGTACCTAGGCTGGAGTG
CAATGGCGCGATCTCGGCTCATCGCAACCTCTGCCTCCCAGGTTCAAGCAATTCTCCTCCCTCATACTCCTGAGTAGCTGGGATTAC
AAGCATGAGTCACCACGCCTGGGTAATTTTGTATTTTTAGTAGAGACAGGGTTTCACCATGTTGGTCAGACTGGTCTTGAACTCCGA
ACCTCGGGTGATCCGCCCGCCTCAGCCTCCCAAAGTGCTGGGATTACAGGGGTGAGCCACCGTGCCCAGCCATTGTGTGGGTTATTT
CTATTGATGTTTACCATATTAGATATTAAGACTGAGATTATTTTAAAATATTTATTAATGCATTTTAAAATAATAAAAACCTATTAT
ATATTAGCATAAATAACATTTTTAAGGAAAAATAGTTATTTTTTTAAAACAAAAATATTTACCAAGAGGGACAACATTGGTTTACCC
TTATTACAAATCTCTTTAATTTGGGACTTATATAGAAGGCACCTGGATTTTCTAGCTACTTTTGCATTAGGGCAATTGTGATAGCAC
ATGTCATGTAGCCTCTGAAAAACTCTACGTTCATGAGAGAAAGTGTATAAAGCAAATAATTTCTTAGTATTATGAAAACAGTTTTGA
CCTTGCAGACCTCCTTATGGACCTCCTGAAAGGGTCTCTTGGAGAGAGAGAATCATTGTGCGGATTCTTCCCACATAACCCTCACTC
CCAGGTCCCAGCCTTTGCCCTCTCCCCTGCCTCTGCATCTGCAGGACTGGCAGCCTCATCCTGTGGGCAGGCTATGAGTTAGATGGA
CCTTGTCAAGTCCTTCCACTTCACTCACAAGCTGGTGTGACCTTGGGCTTTGAGTAATTCCCTGAATGTCTCTTGGCCTCTGTTTTC
TCATTTGAACTTAGAGATGAAGAATTCCTCTAGGTGCTGTTGTCAAGTTCCATGAAACGGATTGGTGTTGATTTTTCTGCACACTTC
CCAATCCCCAGACGTGGGCAGGAAGCAGGCAGCAGGGCAGGGTAGACGTGGGCTGGACCTGGGCCCATCTGTGGGCTCCTCACTCCT
CTGGGGAAGGCAGTGGTTGCACAGGGGGCTTGAGCCTCAGAGAAGGTACCTGTTGGGGATGAAACCCGCTCAAATCTCCTAATAGGC
CTGGATATGGAGGGTGAACGCCTGGCCCTCTGGGTTACCAACCCTCAGGGTTATGAGGTGCTAGACAGGAAAAGGGTGGGCCCTGAT
CTCTGCCTCTCGCCAGCCAGGCCAAATTGGGGAAGTTCAGTAGTCCCCCAGTTTTTAGCAGGGTTGAGTGGGCTTTCTAGGGACTCT
GTCTCTGGGCTGTGAGTTTGCTGAGTCCTGCTGCCCGTTCCTCTCGGCTGGGCTGCGTGTTGTGGGAGGCCCAGTGTGCTAGTAGCC
CATGGCAAGCCCTCCCTTTAGCCTGTGTCCTGTCTGCCTTAACACCAGGCTTTGTTTTGGTAAGTGCTTTGTTTTGGAATCTTACCC
ACCGGTGGGATTTGGGTGGCCCTGTGGGGTGGGGTTACTACCTCTTTTCTTCACCTTCCCCCTCCATTGGCCCCAGCCAGTGGGGTC
TGCAGGAGTCCTGACTCACCTCCCTGATTCTCGTCACCCCCAGCCCCACTGAGCCCCCTCCTCCTCCCCAGAGCAGAGCCAGTGACC
TGGGACACAGCTTCACTGACACTCAGTCTGGCTCTGCCTGCTTGCTCACACACTGTCCGTGTCGGCCTAACTCAGGCCTCAATTTCT
CCATGTATTTAAGGCCCTTTCTTGTGTTTTATTTTACCTGATTTGGCTTTGTGTGGCTTTCCTTCCTTTTATACTAATGCTTCTCTC
TGATCTTATTTGCATTCAAATTACCTCGCCAGGTGGTTCACCAATCAGAGGTAAGAATGCTGTCCGTGCCTCTCGCCACACCACGCC
TTGCCACTGCCGTCACCTCCACTCCATCCCGTCCAGTCTGTCACCTCCCCATCCCCATCCCAACCTCTTCAGCCTCCTCATCTCCAG
CTCCAGCACCCCCAACACCACCGGCCACCACACACATCCACTCTCAGTCATTCCCTCCTGTGGACTCATCCATTGCAAGTCTGAATT
GTGAAGATACTCGCCGGGCCCCGCCTAGGACAGCAAGCCCTGCTGCCCAGGCCTTCCAGGCCATCCCCACAGAAGGGACCCCATCAG
CTCCTACTGTGAAACTTAGCCTGTCCCCCGGCTTCCCTAGACAGAAGAGCTTCCCTTGAGGTTGAGGAGTGTGACCAAGCCTTGCCT
TTTCTCTTGACAGTCTCTTTTGGGGGGACCTCAATTATTACTTGACTTTCCCTTTAGTATCCCGGCTCTGTACCGTGGTAGAATGAG
GATCATCCCTTCAGCCTGGCAAGGATTAGGAGGAATGTTCACTTAAGAGTTCTGTGTAGGTGCTAGGATGACAACAGGGAGGTAAAC
AGGCTCGTGCGTAACTCCATCACCTGACCTGTCAGCTCGGAGTCCCCGCTAGGCTCCCTCCACGCAGCAGGGCCCTGCAGCCTAAGA
GTTAAAAGCACAGATTCTGGACTCAGGAAGATCCAGGTTCAAATCTGACTTACCACCTATGTGATCTTCACAAACTCAGTTCATCTC
TCAGAAGTGTATTTCCTCTTTAAATTGGGTCACGGCCACCTGCCGCACAGGGTCATGAGAATTAGAGGAGAGGAGCATGTGTAGTGT
CTGGCGCAGAGCAGACACCTGTAAAATGGTGGCTTGTCTATTCACATCATTTTCTCTCCAGTTCTCTCAGTGTCTGGGCACATCTAG
ACCTTCAGAGCTCAGGACCAGGATGGTCTCAGGCAAGAGCAGCTGCCTTCCTGGGTGCTATCTTGCCCATCACCCGGAGGCTGGCTC
TTGCTTCACCTGGGAGCCCCCCAATCCCCCTGCCCATCTACTCAGCCTGTGGTGACTCTTTGTTGTCCCTGCTCCTGGGTCCTGGTG
TTGGTTCCAGATTTGGGGATTTCTGTATGCAAATAGGCACCCAGTCTCTGTTAGGCCCCCTGACCACTCTTAGGCTCTTGTTGCAGA
GGAACCTCCTATTTTCTGGGTCAGAAATTTCACCCAGGAACCACTTTCTGCCCCAAGCCTGTGGCACCAGCCACCAGGTCTCCCTCA
CCCCACCCACACCCCCCTGCCTCACTCAGGTGACCTTAGGGCAGCCTCGACCCCTCAGCGGCCTGAACCCGACCTGCTTGAAGGAAC
ATTTTCTGTACTAGTGTCCATCCATCCCATCAGCATGACCTGTTGGGTGCCCATCAGGGCTGCTCTTAAAGGAGATGGTATTTGGCA
TGTGCACTGGGCCCTGGTGCTCCCTGGCATCTGGTGGGGAGGGCTGAGGCCAGCACCAAAGAGGCAGGCTGGTCCTGCGTCCTGCAT
TGGTGGCCTCCCCTCTTCCTTCACCTCCAGCTGCCAGCCCCCTGGGGGTTCCCAGTCCCTCCTGCTGCCCTGGGTTAGATGGTGGAA
GAGGGACATCAGGTAGTGAGGTGGCTATAGGTCAGACCCAGGGTGGTACCCCTTTGGGCCCCAACCGTGAGAGCATGACTTGGAACT
CTTCCTCTGGCTCCTGTCCAGTCCAGGCAGGGGGGGCCCGGCAGCTTCTGTCCATGTCCTGTGTGGGTGTGATTGTCCATCAGCCTG
GGCCCTGGGAAGGTGGGGGCTGTCAGGTTAGGCTGGCTGTTCTGTGCAGGAGGTGGTTTTGTCAGACTGTGTCCTGTGGGGGTTCTT
AACTGTTGGACCGTCCTGTGTGGGTGTAATTGTCTGTTGGATTGTTCCAGGGAGGGGTGTGCTGCCTGTGCCCGGGGGTGTGTCTGT
CACAGACAGCTTGCCCAGTGTGCTTTCACAAGCAGTTTTTTCAAGTGTTTATTGGTGGGGCCAGGGGACGGCCAAGTCCAGTGGATC
CACCCCCACCTTGCCGCTCTGCCTTGGCCTCCCCCGCCAGGACCTGTCAGAACTGGCCTCAAGCCCAAGATTCCTCCACCCTTTCTG
AGGGTCCTTTTGCTACCCACCCCGATCCTGCCTCCTGTCTGTCCGTCTGCGGCCAGAGCAGCCCCTCAGGGCACTGGCCCGAGTGAG
AGCTCCAACCTCCATTAGCCTCTTGAATTATGCAGGAGCCGTGGTGGGTCGAAGCACTTTAGCAGGACCGGGCTGAGGAGTGGAGCT
GGAATGGGTGGGGCTGGCGGGGAGGGGGTGGAGTAGGAGGGGAAGATGGTGCTGGGAGCAGAGGCTGCTGGGCACCAGGCCCCCAGG
GCAAACCCATTCCTTCCTGGTGGGCGGCAAATTCTCAAGCTCCAGCCTGATGAGGGCATGGCGTGCTTGTGAATCCCAGGCCCGCCA
GTCTGCAGGCTGCTCCCACACCTGGCTCTGACTGGCTTAGTGGGGCTGCGGGGCCAGGAACACTAACAGGGATGTTGATTCGGAAAT
AGGCTCCAGTACTGGCAAGAGTGTGGCAGAAACTGAATGGAGCTGGGGGTTGGGGTACAGTCCCTGGCAGAGCAAAGGTGGGTCTTG
CTGGACTGTGGGGAGCATGAGATTGTGCTCCAAAGCACAACCGTTGGTTCTAGACAGGAGGCCACTGGGCTCTTCAGGGAGAGCTTC
CTGGAAGCAGCAGGGGTGCCAGGGTGTGGCCTGGATGAGCTTCGACATCTGGTCAACATCAGCCACAGATGCTGTCGAGACCTCTCA
CCGTGTTCCAGGCCACACTCGACATGGGCCGTGCCTGACCTCTCGCTTCGTGTACCCACAGGTGCCTTGCAGATAGAGAGCAGTGAG
GAATCCGACCAAGGCAAGTACGAGTGTGTGGCGACCAACTCGGCAGGCACACGTTACTCAGCCCCTGCGAACCTGTATGTGCGAGGT
AAGGACTCAGGCAGTGCCTGGCCCCTGTCACCACAGAGCTGTGCTGCACCTGCCGGGCTCTCTGCCCAGAGCCCTTGGTGCAGACAC
GCAAGGGACTGCCATGGGCCCAGTCTCTTCTCCTTCCTGCTTCTTTCTGCAGCAGCAGCAACAGCTCCCACTGGGCAAGTTCCTGGC
GTCTGCCACTACTTCGCCTTCCTTCCTTGCAGGCCCATGGGGAAGCAGCCACTCTTGGGAGCATTTGTATCTTTTGTAGGTCTTGCC
GCATGGGCCCGGAGCCCCATGGGAATTTGGAGCCATCCAATCCGACTTCTTGGTGTATGTGCATGTGTGTGTGCACACACGGGCACA
CTTATCTGTGTGTAAATGCATGCATACCTGGCCTGGCTTTCCTTCAGTACATCCTCCTGCCTGCCGCAGCATGGTGGGCTCCAGAAC
CACCATGGCTCTGGGCTTTGGGGTAGTAAGGCCTCAGGAACGGACCAGGCCAGGTGAGCCTATCCTCTGGCCAGGTCTTCCTGGAGC
CTTCCTGGAGGAACCCTTGTGTTCAGCTGTGAGCCCCTGTGGTTATGTTGCCTCGGGTGAGCACCTGGTGTGTTCCAGATGTTCTTT
GCTGGGAGCGAGTGGACAGTGTGCTTTCTGGAAGTCCAAGTTCCCAGCAGAGAGGAACCTGGGGACTGTTTTGGGGTTTCCAATCTC
TGCTATGCTCACAGCCTAGGGGTGACTAAAGCTGGCCACCTTGCCCTTTCCTGAGAACACACTGGGTCTGGTATACATGTGCCCAGA
AGGGGCCCTGAGAGCCCCTTCTACCCTCATACTGTCCCCTCACCTAGCCTTCTTGTACAGATGGATGTCATTAAAAGTTAAACTGTT
GACCGGGTGTGGTGGCTCACGCCTGTAATCCCAGCACTTTGGGAGGCTGAGGCAGGCAGATCACGAGGTCAGGAGATCGAGACCATC
CTGGCCAACATGGTGAAACCCCGTCTCTACTAAAACTACAAAAATTAGCTGGGCGTGGTGGCGCGTGCCTGTAATCCCAGCTACTCA
GGAGGCTGAGGTAGGAAAATCGCTTGAACCAGAGGTTGCAGTGAGCCAAGGTCGCGCCACTGCACTCCAGCTTGGTGAAAGAGCAAG
ACTCCATCTCAAAAAAAAAAAAAAAGTACAACTGTCTCCACCCAGGGGATTGTAAAGGAAAGCGGACATGGCTCCTTGCAGTGATGA
TTCCATCACGCCAAGTGCTTAGCGCGGCTTCCTGAGGGGTGTCGTCTGACTCTCCACCCAGTGCACGTGGTGGGTGATGGCGGTGGC
CTGTCCGACTCTTTGGGGCCATGTGGTCAGACTTGCTTACTCAGAGCAGAGAGAATTAGGCTGTCAAGGCTGTCAACCAAAATACAC
TTATCAAGGAGTTGCTGTCTGTCATGGGAGAGGGTGGTTCTGGAGAGGCCACAGGCTGCCAGCCCATTCGGTGACCAGTTGAGCTGC
AGAGGAACTATTTGTGGTGGGCAAATTGTGCTTCAGAGACTATTAAAGATGCTTCATGAGAGTTGGGGGCATGGGATGGGAGGTCCC
CTAGCTTACTGTCAGGTGTCTTACTCCTCAAGGCTCAGGCCTCAGGTCAGCTTGTCTGTCTTCAGGAACAAGAGCCTGAAAACTTCC
CAAATGGCCTAGAGGAGTGGAGTTGTAAGCGGCACAGCCTTGGCCCCACCCTGCCCCAGGCAGAGTGTCTGGCAGGCATTGGGGCTG
GCGAGTCCACTCCAACAGAGGAGCAGCTGGAATGACCCTTCTCAAAGATCTGCCTCCTCCCACCCTGACTATCGCAAATGCTGTGCT
CTCTAGAGCTCTGCTCACAAGACTGTAGGGTCGAGAGTGGCAGTTCCAGGCCTCAGCAAGAAAGCCTTTGCATGTGGCTGCACCAGG
GAGAGGAGAGGCCCAGTAGAGCCACTGTAAACCCAAGCCAGCTGCAAGGGCGTGGCCTATGGGCACATTTCCCAGGGCACAGCTACA
GGAGGCAGTGCTGAGGCAGAAGAAGGCCCAGAGGTGGATGGACCCCCGCATTGTCCTCTTCACTAGGCAGCATTCTCTGAAGTCACT
GTTACACTGGCCCCAGCCCTGGAGGCAGAGCAGTGGGTCTCTTTGGAGATCGACTGAGCTCAGCTGCTGTAGCTGGCAGCTCAGTCA
CTGCCCAGGGAGCTCTTATGTGCAGCCCCTTGGAGCATCCCTGTCTGTGGGGCTCCCTGTTTACACATGGTAGACTCAGGCAGGGCC
CTGACTGGCTCTTGCCATGGCCCAACCCCTAACTCTACCAGCTCTTCTTGAATCTACCCTTCTGGCTCCATCCCCACATGCCTTTTG
GTAGGAAATACGGGGGTTCCTTTAGTTCCAGCCCCACTTGCCAGGGACTCCAAAACAGCCAAACCTGACTTCTGTTGTCTCTCTCAC
ACACACATACGACGTTCCTCTAGGCCCTGATGTCTCAGGACCGTTTCTAAGCTCTGTCCCCAGGACCTGCCTCATGTAGACCCTAAT
TGAATCTACACCACCCTGCTTTGCCAAGAGCAACCTGCCCCATAGGCCCTACTGTCCTACTGACAGGACTTAGGCACACCTGAAAGG
CCCTGCTCCCTGGGTGCCTTGCCTCTGGTGCCCCCACTGCTCGTCATCTCATGGTTGGCAAAACCCCTGCCTTCTGCCCTCACCAGT
ACTAGGGGCCCTTCAGCCCCAGCGCCTGGGGACAGCCTGTACTGGGCTGACCACCAGCTCTGCATCCTGAGCCCTGCTTGTGGGTGG
ATGGCCGTGGAGGAGTCCCCAGTGCCAGCCTGCTTTCTGGCCTGCACCCCGTCCCATCTCCCAGCAGAGAGGCTTCGAGGTTTCCTC
CCACAGCAGGCTCCTGAGCCTCAGACGGCTTGATGTGGGCTCTAGCCCTGAACTTCTCAGCCGCAGCACTGTTGACATTTTGGGCTG
GACGATTCTTAGTTGTGAGGGGCCATCCTGTGCATTGGAGGGTGTTTAGCAGCATCTCTAGCTCACTAGATGCCAGTGGCACCCCTC
AGTCAAAACAAACGTTGCAACGTGTCACCTGGGGGAAATTACTGATGGCTTCCCACTGCCATTGTCTCCACCCATCTGACTCTCTTC
ACCTGTGTCCACTGGCCGCAAGGCTTCGTCTCCTCATCAGACTTGGCTTCGGTGTCTACCTCACATCTCCCTTCTGTCCCCCAAATT
CTCAGGGCTCCCAGTGTCGCTCTCTGAGGCCACGCGACTGCTACATTTAATGGGACAGTACACGTGAAGCACCAGGCACATTGCCAG
GCACACAGGAAGCACTTGCTAGTCAGTAGCCTCTGCAGCTAGCACTCGGCTACTAGTCAGTAGCCTCTCGGATAGCACTGTGGGGGG
ATGTGTCATCCAGTTACATCTGACTTTGTTCACAGTTGCCTGCAGCTCCACCCACAGTCTAGCAGGCCCAGGCATGCTGCGTGGGCC
AGAGCCTTTCTCCTTCACCACCTGACTCTCCCTGAGTGACTCATTCTCTCCTTCCATCTACAGCTCTCTGGGTGTACAGCTGCTGGG
GCCAGGGTGGAGCCCTGCCCTCCTCAGAGCCTGGCCTACCTGTGCCAGCACTACCAGCCTTCCCCCTTTCTCTAGGGACCTGGCTGC
GGGGCCACAGCTGTCTAAAACAGGGACAGTGCCTTTTTCCCCACAGGTGCCCAGACATGCTCCTTACACCGGTGGTGTGTGTGGGGG
TGGCTTCTAGTGGCTCCTGTACCTTGGCAGGTTTGTGGGCTGGGTGGGCCTTGACCCCAGAGCCCGGTCCACAGGGTCTGTCTGAGC
TGTGGGGTGCGTGTGAGGCATGGGGGCCTGCCTGTGCCCCATTTTCACCTGCCCCGGCCCCACCCTCGGCCTCCCTGGCGCCTGCTG
GCGGGCCTCAGCCCTGTCCACCATGTCCTCCATGAGTCCTGAGTCTTTTGTGAGTGATGTGGTTCGTGTGCACCTGTGTGCATGTGT
GTGTGCGAGGGGGCACAGGAGTCTCGTCTCGTCTCCGTGCTGTGTGGGCAGATGAAGGTTGGCCTGTTTTTACTCTCTCTGTGTTTC
TCCTTGTCTTTTTTTTATTCCCTCCTCATCTTCATCGCACTCTGCCATCAACCCAAACTCTCATCTCTCAGATCAGCGAGAAGGTTG
GTCCTTTTCACTTCTTATCCATCTACAGTTCGCCCATCGATGGGACACGCCTGTCAGTAGGGCCCAGCGGGCTCGGTCAGCTCCCTC
AGGCTCACTGCGCCGTGCCTGCCTGCCAGTCTCTGTTGTTTGGGCCGGCGGGCAGGCAGGACCAGGGATGGGTGGGCAGACCCCTGA
CCTGCATGTTCCTGCTGCTTGGGTCCCTGGTGCACCACGTGTCTGCATGTCCCCTTAGCCTGGTTCCCCTGCAAGGGTGGTGGGGTG
GGCATGCTGCATGGCATGAAACTGTACCCCACCTTTGCACCCAGGCCGGGCCAGCTGTCTGATCCAGGCGGTGCTCAGGAATGGTTG
TGGGGGGCTGTGGTCAGAGAGAGGTGTAAGACCTGTAGCGCTTTGTGTGCACCTGTGGGACCTTTCACCGCCCTCCTCGCCTAGAGA
CCCAGGCTCAGCCGAAACTACTGGAACATTAAGGCGAGAGGCTAGAGCAGGCTGTCCTCCCAGTATCCTGCAGCACAGGGCAAAAGT
CCTGCAGTGTGTACCCTGTCCTTGAGGCCCTCAGGTCCCCCACCAGGGCTTAATGGGGCAGTCAGCCCTGGTCTGCCTCAGGGAGCT
TGGTGTCCATGGCGCAGGGTGAGTAGCGTCATCTTGGCACCCTGAGGAAGTCTCCCATAAGGTTGTCTTTTCCTTCTCCAGCCAGAT
CCAAATTAAGATCCTCCTACCTGCCCCAGGCCCCTCTGCCCAAGCTTCCCAGCTAGCTCCCGTGTCTGTTTGAGGGTGGAATTTAGC
CCACCAGCCAGCTAACTGACATTCCTTAGCTTGATGCTTTGCATTCAGACTCAAGTTGCTTCTACCTGGAGTAACTGAACGGTCAGC
TGCAACTTAGCAGGATCCATATGGGAGCCTGGAAGTGGCCCCTGACTCAGCCACAGGTTAGGAAGGGTTTCCTGGAGACTGTCCTTG
AGCCCATTCCAGAGCAGGTCTGTTTGGGGCTTGGTTTTCCGCCCCACCCTTGGAGCCAGACCCCCACCACTGATGCTGGGGTTGGCA
GCAGGGGCTAGTGGTCATTCATGTATCACATGGTCTGTGTGGTCAGAGGAGACTCACCTGTCCATTGGGTTTTGGGGGTTCAGGATA
GCAGTGTAGGACAGGCTGAGCCTCAGGGAGCAAACCCAGGCCATCCTCGTTCCCTCTGCCACATCCTCCGTGGCTTGAGTTTCCTCT
GTGAGGGTGAGGGAGCCGGTCGAGAGGAGGGCTATAGCCTAGGCTCAGATATCAGCCCTGAGCATCCATCCATATGCACAGCTGAGC
ACTGGGGCATTTCTGTTACACCATTTTGCTGACCGGGCAGATCTGCGAGGGCGTCCAGAAAACAGTCATCAGCAGAGACAGCCCAGC
CCAGAGTGGCTCCTTCCATGCCCCTTCCTCTACCCTCCTGGTCCCTCGTCCCCTCCCTTCCCCCACCCCACCCTATGGTCCCACCAA
AAATAAGCACATGGACAAATATTTAGCGGGAAGGCAGACATGAGTTTCTAAATATTCCAGGGGGATTTGCCGGGCTGGTAATTTGTT
TGGTGCTGATAATTGCATCTTACATTTTTCCAGCTTTACTTAAAACTGTGTGCCGGGTTTGCCGGCATTTAATTACTGCTCTGCGGC
AGCCACAAGGTTATTTATTAAAGAGTTATTTTATCTTGATACAGTGGATCCTGCCCCTCATCCCCTCCTTAATGTTGTGTTATTTTC
ATCAGAGAAATTTCTCTGAGTGAATGCAGATTGCGGGGCTGCCTCCCCCTGCGATTAGGCTGTCACTCTACTGAATGCTGATGCCTC
TGGGCGCCGCACCGCCCTATTATAGGGGGTTGTCAGCGCAAATAATTAGACTTAAAAGTTACAGCTGAAATATAATCCAGAAATGGC
AGGGCCCTGTTTTGGAAATTGTCTATAAAATGTCAGCACTAAGGATGCACAGGGAACGGTAATATACTGGCATTGTTGGAGCCTAAG
ATTAGACCCTGAGTGCATTTTTCCCAGCTCCAGTTTTTCTTTCCCTGCTGTTGCTTCTGTCAATAGACTGTCCAGGGTGAGGCCTGT
TCCTTTTTGGAGGCCCTGTGCCTGCAGCAGTGGGTGGAGGATGGTTGGGGCTGCTGCAGTGAGATGCCAGCCACTCCAGCTGTTGGC
ATGGGGGTGGCTGTCTGGGCAGGTCTAGGATGCGGGTCCCTGTTGGCACTTGGAATACAAGGAGGTTGTCCCTTGCCTGTTTCCCCC
ACCCTTCCTCTGTAGACATACCCTGACCTCCCTCACATGGAAAATGTCTCAGTCTCAGTAACGTTTCTGCGTGGCTGAAGCCCAGTG
TCCTTGTGAGAGTGAAGGTGGTGTGCCCAGGAGCCTCTGGACCTGAGGCCCTTGCTTGGTGTTGCAGGTGACCCTAAAGGCCACTGT
TGACCTCTCACACTTATGCCTAGCCCCTAGAGCTCTCCCTGCCCCCTGCATCTAGGAGGGAAAAAATAAACACAACTCTTCTGTGAA
GCGCCTCAATTTCCTGACAGCCGCAGGAGTTGGAGGCTTTAGAAGACGTGGCTCAGGGCGTTGGCCTGACCTCCAGGGCTGATCCAT
GAAGTCCGCCTTTTGGCCTGAAGACCCTGTCCTCCAGGGACAATTTGGGGGTAAAGTCCAGTCAAGTTTCTAGGTTGGTGCCCAAGG
TTTATTGGCCAAGGACCTACTTCCTTCTGCTGGGGCTCTTTGTCCTGGTATCCAACGACGTGATTAGGCCCCATGATTCCACCACCG
ACTGGGGTCAAGCCTGCAATCATGACCTCTCCCTGGCACAGGCTGTCTAAACCCATCCTTACGCCCGCTGGAGCAGCGTCCTCTCCT
GCCAGCCTCTGCTAATTCTGATGCTTCTCCCCAGAGCCTTCTGGGTCTTTTTTCAGGCCTGTCAGCTAGGGCTCGAAAGGGTGACAG
TTTTTCATCACACAGACCTACTCCTACTACGTATATAAACTTGGTCATGTGGCTTGGTTTTAAAACAGTTCCCTTGACTGTAATTGG
CAGGTGATAGCATCTACAGCATGGAAACCTGCACCTAGTAGGTGCTTAAGCATAGCTTCCCTTCCCATGAAGAGGAGGAGACAGGGC
ACCCACTGGCCGAGAGGACAGGAGAGATTTAGTTCATTAAGGCTGCTCCTCTGTGCTGCCCCCACCCCGCTTCTGTGGCAGGCCTGG
ACCTGATCTGCAAACAGACTTGCCTGCCTGTGCCTGTCCCTGAGGCCCATCTCCAGAGCAGAGGGAGGGCATGCACCGCTGGGCTGG
GTGGTGGTCCTGGCTGAGCCTCCTGCTCTCACCTTGGACCATTTGACGTGCGTGCTCAGAGAGTCCTTTGTTGCCATGAGACACTTG
CCAGCCATGCCCCGGGATCCACCTGTTCTGCCACAAGAACTGTGTCTGTGACATGCTGTCATCATTGGTAGAGACTCCTGGCACTAG
AACAGATGACAGAAACCCACTTATACCAGCGTAAGCAAAACAAGGAAGTCCTGTGTTACAAATCTGAAAAGTGTAGTAGATTTGGTG
CTGGGCAGGGCTAGATCCAGGTACTTAAACAGGTTGGCCAGTCATTTGTCTGTCTGTTTCTGAGCTCTCCTCTCCTCTGTCGACATC
TTTGTTCTCAAGCAGCTTTTCCTGTGTGTTGGGAGAAGCCATCCCCAGCTGCTCTAAGCTTAGCTACCTCAGTAGAGAGAGAAGGTC
GCTTTTCTAATAGTTTCTGCGGAAGTCCAAAGGCTGTTTCTCCATAGTCAGATCTGGGTCACACACCCCATGTATGAAGAATGCAGG
TAGAAGTGGTTTCCTTAAAGACAATGGGAATGCTCATTGATTTGAGGAGAAAGCAGAACCCTGAATGTCTACAAGATACTCTCATGC
CCTAGGTGAACCTGGGGACACAGATCCAGCCCACCAGCACCTGTCAGGCCTGGTCTCTGGGGCTGCCAGGGTTTGGGTTCTGTCTGG
AGCACCAGAGTAAGGCAGGCTGTGGAAGCAGTTGAAGCAAGTGCTAAATTAGGTAGGGTCTCTTGGGCTGGGCAGAGGAGTCACTGG
GAGCCATGCAGGAGCTTGTTAAACAGGGAGTGGCATCAGATTTGTGCCAGCTGTTTGTGGAGGGCCCGTGAGCAGGAATTGGGGCAG
TCGACAACAAGGCAGGAGGACTGACGCAGAACACATTGCAGCAACTCAGGGAAGGATGATGATGGCTTGGACTTTGGTGGTGGCAGT
TGCCTGTGTGTATGGAGAGAAATACATGGATTTGGAAGATATGTTATGCCACGCTGTTGACAGGACCTGGTGACTTGCTGAATGTCT
GATCTCTGGGTCTAAGTTTTGACCCCTGTATGGTGAGGTCAGACGTTGGACCGGGAGGTCTCTGAAGTTGCTTCTAGCTCTAGTTCT
GTGATTTGGGGCTGTGTGAGCAGCATAGTCTGGTGCTTATAGTGGAGATGCTCTTTATGTGGTGCTGTGGGGTCTTTGCATGCCTGG
GCCCCCCAGCGAGGGTCCTCAGAGACCAGTGCTCTCAGCAGTGCTCTTGGGTACTGACTAGCCTGCTGCCTGCCGCCTTTGGCCCTG
CGTGTGAGAGTGCCCGTGTAGGACACACCCTGGCCTCTGCTCACCAGCTGCATGGCCCATCTGCATGCCAGACATGCCTGCCCACGG
CTGGGTTTCATCACCTCCCTTCCCTCTGCATGCTCCTGTGTCTGTGTCAAGCTTCCAACTTCGGGGGCGGGTGGTTCTAGGCTGTAC
CCTGGACTGAGAAGCTCAGAACAGCTTCTGGTCAGGGGAGTCCAAGGCTGGAGGTCGTGGCCAGGCACAGCCACAGCCACCCCTCCC
CTTGAGACCCTTGTTTGTTGCCGCAGGAAGCCGTCTGTTCGGCGCCCTCATCATGTTACTGCCATCGTCATGCCCATCCTGCCCGCA
GACCATGTCCCTGGCCTGCCCTGACCCCTGCCCCTCCCCAGTCCTCCTGAGGGCAGCCCTTTTGCTTGGGTTCCCAGGTTGTGCCCT
GAGCATGGCTGAGACCCTGGCAGGCGGCCCTGCTGCTTGCCTTATTTCTTCCCCTCAGGCCATGGCCTGGAGGTGGAGGCAGTGACT
CCACGGCAGGTGGCTGTGTCCCTCTGGACTGGCCTTCTGCTCTGCCCAGCCATGCTCTGAGGACCCTCCACCCTAGGGCCTGCTTTC
TCTCCTTGGTGCTCCAGCCAGGAGCAGGCAGAGATAGGCCCTGGAGAACACCCTGGGTTGTGGTCCTGACCAGGCCTGGACCTTGTA
CTGAGTCCCTGTGTGACCCTGGGCAGACAGGACCTTCCTGACAGGCCTCAGTTTCCTAGGCTATAAAATGGGAGCTTGGTTGCAAGA
TCTCTCATTGAGTAAGTCATCGTGCtCCAGACAGTCCCTGAGTGTGGGGGCCACTTCTAGGCTGGAAGTGGGTGGGGTGGGAGTGGA
TGATGAGCTGGGGTCTGGCGGTGCCACCCCTCCTGTCTCTGAGCATGGGTTTGGCTCTGACCAGAGGGGCTTCCTGAATGAGGGGCC
CCTGCCCTTCCATGCAGTCGTGTGTCCTGCCCGGCCTGTGAGTGCCTCTCTCCCTCCTCCTGCAGTGCGCCGCGTGGCTCCTCGTTT
CTCCATCCCTCCCAGCAGCCAGGAGGTGATGCCAGGCGGCAGCGTGAACCTGACATGCGTGGCAGTGGGTGCACCCATGCCCTACGT
GAAGTGGATGATGGGGGCCGAGGAGCTCACCAAGGAGGATGAGATGCCAGTTGGCCGCAACGTCCTGGAGCTCAGCAATGTCGTACG
CTCTGCCAACTACACCTGTGTGGCCATCTCCTCGCTGGGCATGATCGAGGCCACAGCCCAGGTCACAGTGAAAGGTGAGTGTGGCAG
GTGCTGTAACCAGTGCCCTCCCTGTCATCTGGGAGGTCCTGGTGGTGGGCGAATGTGAGCTGGCTGCCATGGGCACAGGCATGGCTG
AGGGATTCTTGCCCTTTCCTGGGTGTCCCTGCCCTGGGGTCCTCCAGCCCTTAGAGGGAGGGAGGGATTTCTGTTATTAGCCGGCTT
AATGATAATAGTTAAGGCATATTGAGTTTCTTGGTGGGAGACATCATGCTAGCAAGCACAGTTGATTTTGTTTTTTTCTGTTTTACC
CTGGGAGATAGGTGCTCTTATTATTTCCATTTTTGAAACGAGGGAACCGAGGCACAGAGAGGGTGTATCACTTGCCCCAGGGTCACA
TAAAAATAAATGACAGAGCAGGGACTTAAACCCAGTGTGGTCTGAATCCATATCTCACCCTCACCACTACATAGTACCAGACCTTCA
GGTTTAATAGCCTCCCAGCAAGAAGGGTGTGGTAAAATAGTAGGGGAAAGTGTGTTCTCTCTGCCCTGATACCTGGGGACAGGCACA
CATGTATTACACATATGTCACATATATGTAACCGATTCTGGCCAGGCACGGTGGCTCATTCCTGTAAGCCTAGCGCTTTGGGAGGCC
AAAAAAACTGGGCATGGTGGCACACACCTGTGGCCCAGCTACTTGGGAGGGCTGAGACAGGAGGATCACTTGAGCCCAGGAGGGTGA
GGCTACAGTGACGCAGTGAGCCATGATCACACCACTGCACTCCAGCCTGAGTGACAGAGCGAGACCCTATGAAAAAAAAAAAAACCA
GATTCCTTCCTGGTCTCCCCGCTGCAATTCTCAACAGCCTCTGATCCATTATCTGTTTTGCAGCTTGAGTGATCTTTAAAAAATGTA
ATTCAGGATCAAGTGACTCCCTTTTAGGAAAGGGAGAAACACCTTCCAGTGGCTTCCCGTTGTTTTAGGATACAGATCAGGATCCTC
ACTGTGGTCTCCCAGGGCCACTGGCTTCCCACCCTGTCACGCGCCAGCTGTCCAGACACATGCTGCTTTCCGTTTGCCGTGCTCCTT
GCCTGGCCCCTTCTCTCACTGGCTCCATGTACCCTTCCAGGCCTCAGCTTGGTGATGCTTTCCTTCCCTCATCACCGCATCCAAGGA
GATTCCCTGGATCTTACTCCACCTCAGCACCCTGATTCCTGCTGTCCTTGCACATGGCAGAATCATGAGTACGATCGACTTGTTTAT
TGATTCTCTCTTGTACTGAGGTGTAAACTCCATGAGAGCAGGGATGTGACTGTGTGGCATTGTAGCCCCAGCACAATGTCTGGCATT
GAGTGGGTGCTTGTTAATTATTTGTTGAATGACTGTGGACTCAGGACCTTTCCACTTCAATTTGAGCCAAGCTGCAGGGTCTGTGGG
GCCAGCAGCCCCATCACTCTTTCTATCCGGGCCAGTCCCTAAGGAAATATCTCCCCTTCCCCTGCCTATTACACATACTTTCCACCA
GGTGGGCTCAGGTGACCTGCAGAGGCACATAGCTGCTACCCAGTCTGGGTGTCTTTCATCTACATGGGACTGCAAGGGTCACATCAC
CTCCAGGTCTGTTTGTTAGATGAGTCTGGTGTGCTGATGGGAGGGTCCAGTGAATCCCACAGTTGATATGTGTACATACTAAATGAC
CCAAGGCCCTGGGTGGGGCACTGACAGGCCTGGCCACTGTCCCTCACTTGTGCCTTCAGAGGTCACCATAAGCTTTTCCAGCCATTT
TTCAAGGTAGGCTGTGGGTATCAGCCACACTCAGGTGACCCCACCAGATATCCTGGATAATCCCCAAGTCTAGGGTTGGTTCCTAAG
GATCTTGACCTCGGGCAGCTTTGAGCCTTCCACTTTGTCTCCAGCTCTTCCAAAGCCTCCGATTGATCTTGTGGTGACAGAGACAAC
TGCCACCAGTGTCACCCTCACCTGGGACTCTGGGAACTCGGAGCCTGTAACCTACTATGGCATCCAGTACCGCGCAGCGGGCACGGA
GGGCCCCTTTCAGGAGGTGGATGGTGTGGCCACCACCCGCTACAGCATTGGCGGCCTCAGCCCTTTCTCGGAATATGCCTTCCGCGT
GCTGGCGGTGAACAGCATCGGGCGAGGGCCGCCCAGCGAGGCAGTGCGGGCACGCACGGGAGAACAGGCGCCCTCCAGCCCACCGCG
CCGCGTGCAGGCACGCATGCTGAGCGCCAGCACCATGCTGGTGCAGTGGGAGCCTCCCGAGGAGCCCAACGGCCTGGTGCGGGGATA
CCGCGTCTACTATACTCCGGACTCCCGCCGCCCCCCGAACGCCTGGCACAAGCACAACACCGACGCGGGGCTCCTCACGACCGTGGG
CAGCCTGCTGCCTGGCATCACCTACAGCCTGCGCGTGCTTGCCTTCACCGCCGTGGGCGATGGCCCTCCCAGCCCCACCATCCAGGT
CAAGACGCAGCAGGGAGGTAGGTGGGGGCATGCCGGCTGGGCAGCCAACAGCAGAGAAGGGGAGGCTGAGGTTGTGGCGGTGCCTTT
CCCCCTCCCTCGGCTGTGAGGCTGGGGGCTCTTGGGAGGATCAAGGTGCCGTATTCCATAGATGTGTGGTCAGTTGGGATGTAGGAT
AAGGGTGTGAGGTTAGGACCTGACTTCCTCGGCTCCCTCCTCCCTGGGCACCCCTGACCTCACGCAGATGAGGCTGACCTGCCTGGT
GTGGGGTGTTGCAGTGCCTGCCCAGCCCGCGGACTTCCAGGCCGAGGTGGAGTCGGACACCAGGATCCAGCTCTCGTGGCTGCTGCC
CCCTCAGGAGCGGATCATCATGTATGAACTGGTGTACTGGGCGGCAGAGGACGAAGACCAACAGGTGTGCAGCGGGCAGAGAAGCAC
TGAGGGGGTCTCCTGGTCCCTGAGGGTCTGTGATGGGCTTAACCCAGGAGGGTATTTTCTGGATTCTGTGGCTTATGTGGGCACAGC
CTCTAAGGTTTCTGCTGGAGGCTTAGTGGTGCATGCGTGTCATGTGGCCCGCACAGCCTGTATGAATCTGGAGTCATTGTCACCCTG
TGCTAGGGGCAAAGTCCCCAGGGTCTGCCTTGGGTTTCCTAGGCTCTGTGGCTCATATGACTGGCAGAGCCCTGAAGTTTCCCCAGA
AGCCATGCGTCCCAGATGGCTCCTGTGGTCCATGTGGTCTGTGAGGTGTCTGTGGCATGGGCTAAGCCCTGAGTTCCTTTTGTGCTC
CATGTGGCCTTATGGTGTGGAGCCAGTCCTGGAGCCGTGGTGGGTCCAGAGGTGTCACATTGCAGCCCATGTTGGGGAACAACCTGT
GAGTGACTTTGAGCTCAGAGCTGTCAGTGAGTGCTCCCTGCTCTTCCCAGCACAAGGTGACCTTCGACCCAACCTCCTCCTACACAC
TAGAGGACCTGAAGCCTGACACACTCTACCGCTTCCAGCTGGCTGCACGCTCGGATATGGGGGTGGGCGTCTTCACCCCCACCATTG
AGGCCCGCACAGCCCAGTCCAGTAAGTGTCTCCCAAGTCCGCTGCCTGTTACACCTGGGCTGGGACACACACACACACATGCACACA
CATCCTTCCCCCTCGACCAGGCAGGTGGGGTGGTCAGGTCTGCTGGCCAAACCGAACTCTGGCCTGGGCTCTGAGTCTGGGCCTCGG
GAGAGGGCCAGGTAAGTGCCTCCCAGTCTGTCCAGTCCAGGCGGCTGCTGCCCTCCCTGCTCCCTGCCCAGCCTCCTGCCCCTCCTC
CCGCCCATGCCCCACACACTGCCCAGGTAAGTTCTGTGTCTGTGCCTCATCTCCGCCCCCACTCTGTGCTGGTCACACTAGCTTAGC
TGATGGGCCATCCTCGGTGGGTCTGCCATGTCCCAGACCTGCTTTGOCGGCTGTTTCTCCTCCCTATGGCCCCGCATCTCGTCTCCC
CATCACCCACATTCCCTTCCTCATACTCCCCATGAAAGTTACCTGAGAGCCATGTGATCACCCTGAGCTGACTGCACTCGGGGACAG
GTCTGAGAATGCGGCCATGTGACCCAGCCTGCCCACGTGTGCCTTGAATTGTGACCACGTGGCCGTGCAGGGGACTGTCTGCAAGGC
TGTCTGTGTGAGACTCTGGTGTTGTGTGTGAGAGACTCCGATGCTGCAGCTGTGGGAGTGGCACTGCCCATGAGTGTGAGTGTGTGA
CCACACCTGACCAGCTGGCTGTGCAGCACCTGTGTATGTGCATCAGTGGATCTTGGGCATGACCCTTGTCTTTGTGGGCGACTCTGG
CCTCATCCTGTAACCGTATTTGTGGGTCTCGGTAGCCCCCCAGACAGTAGACTGTGTGTGTGTGTGACTCTGTGGTTGCTGCTGGGC
GTCCTGTGTGTGGTCACACAGCCACAGTCAGCTGCTGCGTGAGACTCTGTGTGATAGGGTGTCATGGATGTGACTGCCTGGGTAGCA
TTGTGGGATTGAGATGGTGACCACATCTGATGTGGCCATGTGATCACCAGAAACATGGCGGTGTCTGAAGTGGTCCTTATGAAGTGG
GTTATGGTCACAAGGTAAGCCACCGTGCCTGGCTCTTCCATCCTACTTTACACAAATGTCATTAAAAGCTCATTTGGAGGCCAGGCA
CGGTGGCTCACACCTGTAATCCCAGCACTTTGGGGAGGCCAAGGTGGGCGGATCACCTGAGGTCAGGAGTTAGAGACCAGCCTGGCC
AACATGGTGAAACTTTGTCTTTACTAAAAATACAAAAATTAGCCAGGCGTGGTGGTGGTTGCCTGCAATCCCACATACTCGGGAGGC
TGAGGCAGGAGAATCACTTGACCCCAGCAGGCAGAGGTTGCAGTGAGCCGAGATGGTGCCATTGTACTGCAGCCTGGGTGACGAGCA
AAACTCCGTCTTGAAAAATAAATAAAATAAAAAAGCTCATTTGGGTAATTACAGGTGCAATGTCTGTCTGCCCTGCTGGAATGCAGG
ATCCCCATGGGCAGACACCATGTGTCTGGTCATGCTGCCAGCCCTGTACCGTGCATGGGGCCTGGCCCCCACCTGCAGCACAGCAGG
TGCTTCTGGAATGAATCCATGAATACAAGGTCACACACAGCCTCTTGACATCACTGGTACATGCACATGTACCCTGCTCCTGTTCAT
TTACCAGTTGGCTCCCACCCTTCCTTCAGGCCTTGGGAAAGTGTCACCTCCCCAAGGCCTGAAGGAAGGGTGGGAGCCAACTGGTAA
ATGAACAGGAGCAGGGAGCACACAGGCCTCCGGGAGAGTCTGCAGGCCTGAACGCCTTCCACCTGCAAGGCACATGGTTATGGTCAC
AAGGTAAGCAGCAGGCCAGTGTGGCTGCAGTAGCTGCCGGATCTATGGCAGTCAGGGCCTTGGGGACACAGGGTAGGCAGGAGAGCT
GACTCTGGCTGCTCTGTGAGAATGGACAGGGCAGCCTGTGAGGAAGCAGAGACCGGTTAGGAGGCTTTGTAGCCGTCCAGGGAAAAG
ATGGTGGCGCAGGTGAGGTGATGGCGGTGGATCTGAGAACTGTCAGGGATGCTGACTGGTCAGACTTGGTAATGGGTTATGAATAGG
GGCCCAGGGGGAGGGAAGTGGAGGTGAATCTTGTTTCTGGTGTATTAACCTGGGAGGAGAGTGGTGCTGGCGTAGAATGGGAAATGG
GGCAGAGGCAGGTGTGAAAACTACAGCTGGTCTCAGACACCCAAGTTCAAGATGCCGTGTGCCATCTACATGGCAGTGCTCGGCAGG
CTGTGGGAATCCAGGGCTGGGCTGGAGCTAAAGGACCCTGAGAAGAAAGACTGCAGATGAGAGGAAATCAGGGGAGGGGAGCGACTT
GGAGGTTGGAAGGAGAGAAAGTTTCAAGAACTAGACGAGATGCTGCTGAGCGGTCAGGAGTACCTGTTGGACCGAGCAACATGGACG
AGTGGCCCTGGAGATGAAGCTTGGTGCATGGTAGGACCAAAGCTGGTGGATGTTTCATGAGGCCTGTGTGGGTGCTGAGAATGTGGA
GGCAGCAAGCCTAGACGTTGCCAGAAAGTGTAGCTCTGAACAAGGGGACCACTATGGCTAGAGAGGGCCGTGGAGCTGAGGGTGGGA
TTTTGTTTTGTTTTGTTTTGTTTTGTTTTTGTTTTTTTGAGACAAAGTGTTGCTCTGTCTCCCAGGCTGGAGTGCAGTGGCATGATC
TTGGCTCACTGCAACCTCCGTCCACCTCCTGGGTTCAGGTGATTCTCCTGCCTCAGCCTCCCGAGTAGCTGGGTTTATAGGCGTGCC
CGCCACCACACCCAGCTAATTTTTTTTTTGTAATTTTAGTAGAGACAGTGTTTCACCGTGTTAGCCAGGGTGGTCTGGATCTCCTGA
CCTCGTGATCCGCCCGCCTCGACCTCCCAAGGTGCTGGGATTACAGGCGTGAGCCACCGCGCCCGGCCTGGGATTTTTCTTTTAAGA
TGGGAAAATTTCAAGCATATTTAAATGCTATTGGAAACAGTCAATAGGAAAAAAAGAAACCGTGGAAAAGAGGGTAACTGGTGGAGC
CTTGGGAGGTGGAGAGAAGGGGAACAGTATGGGTGGTGAGAGAGCTTAGCCTTGGACAGGGTGGCTCAACTCTCCATTCTCATGGGT
GGAAAGGAGAGAATTGCTGCAGATGCAAGTACGGTGGGGGGGTTGTAGCAAGAGATGGAGGGAGCTGCTGTGAAAGCCTGGTCTTCT
CTGCAGTGTGGGAGACATGGTCATGCGCCAAGAGGAGGCAGGCAAAGGGAGCTGGAGGTCTGGGGAGCATGGAGGAGGGGGACTGTT
TCATGTGAGGTGGCAGAGCGAGCCAGGCAGGGGGCAGAAACAGGCAGCATGGAGAGTCCTGGAGGATGAGGCCTCTAGGGACGTGTG
CATAGGACGCCAGCCTGCCTCAGTGCCGCTGTAGAGAAGGAGGGGGAGAGCTGGATTCATCTAGGGCTTGGCAGCGGCCAGGAAGGA
GCATGGAAAGATCAGGAGGCGAGGGACCTTGCGATATTGGTGAGAGTGTGGTAGGTGGTAGACTGTGGAAGCTGAACATGGAGGGTG
GGAAGGACAAGGTTGATGGACAGGCATCCTGAGGAGCTGGCGAGCAGGCTCAGTGCCCGGGGCCTGGAGTGACTGAGCAAGAACAAG
GGGGTGGGGTCTGCAAGGGGATGTGAGAGCCCGTGATTCTGGAGGGAGGGCCATCCCCAGTACTGGCAGCCTCCAGGGCATGACCAC
AGCTGTGCAGGGGAGGAGAAAGTCCTGGAGATGAGGGGCTCTGGGGCCAGTGGGTCAAGGGGCTGCTGAAGTCCCCATGGCAGGGGG
TGGTGACAGAACTTGAAGAGGAAACCTGTGCATTGCAGCAGAGTCCTCGGTGGGCGTGGAGTCTGGACACATCACTGGACACAAGGA
GACACGTGTCTGCCTCCTAATTCCCAAAGAAGCTGTTGAGTGAGGGCACCTCTGTTGGTGGGTACTTTTTCTCCACGCCAGGGGCTG
TTGGCCTTCATGTTCGGCCTCCTGTTGGTGATTACCTGTGGCACTGGCAAAGAGACTGTTTCCAGGCAGGCACAGGTGAAGGCTGCC
TTATGCCAAAAGGGCCACTGGGGCCACTCTTCCTCGGAAGAGCAGAACCGTGGGCACAGCAGATGTGAAGCAGTTCTCCATATGTGG
CTGTGTGTGCGGGTGGCAGTGGGATGGCCACGTGCTTGTGTGTATGTGATATCGTGTATGTTTTGTGTGAGACAGCATATGTGTGGG
AGAGACCTCGTGTGAGAGATGCTGTGTCAGAACTCTAAGACATTGTGTGTGAGAGTGTGTGTCAGATGCCATGTGTGAGACACCAAG
ACACGGGTATGTGATACTCTGTGTATATGTGAGTCTGTGTGAGAGACACTGATACTCCAAGACATTGTGTGTGTGTGACACTGTGAG
ACACCAAGACAGTATATGTGCGAGACAGCCTATGTATGTGACAGTGTGTGTGTGAGACACCATGAGAGACTGTGTATTTACTGTGAG
AGACTGTGTGAGAGACACGTGTGAGACACTGTGTATATGACACTATGTATACGTGAGACTGTGAGACACTATGTGTATGTGACACTG
TATATGTGTGAGACCGTATGAGACAGTGTATATGACACTGTATATGTGTGAGACTGTGTGTGTGAAACACTATATGACACTGTATAT
GTGTGACACTGTATATGACACTATATGTGGAGACTATGTGTGAGACACTATGTATATGTGACACTATGTATGTGTGTGACACTGTGA
GAGACACTGTGAGACACCAAGACAGTATATGTATGAGACACCCTGTGTGTGTGACACAGCGTGTGACTGTGTGAGACGTGTGTGAGG
CACTGAGACTGACACTGTGTGTGAGGCTGTGTGAGAGTCAGTGTGTGTGATAACTGTGTGTGTATCACCGTGTGTGTGTGTGAGAGA
GGGAAGGAGAGAGAGGGAGGCAGGTCAGAGGGAGTCAGGCGTCCTTGGCGAGAGTGGCAGCAGCCTGCCTGGAGGGACCCTGGGATG
GCCATTTCAGCACCTAAGGGGTAGCCTGCCCGGGTGAGTCTCCAGTCCACTGTGACTCAGTCATTGTGCCTGTGATCCCCACCCTCC
ATCTGCTTGCTTCCCCCCCATTTGTCTTCCCCAGCCCCCTCCGCCCCTCCCCAGAAGGTGATGTGTGTGAGCATGGGCTCCACCACG
GTCCGGGTAAGTTGGGTCCCGCCGCCTGCCGACAGCCGCAACGGCGTTATCACCCAGTACTCCGTGGCCTACGAGGCGGTGGACGGC
GAGGACCGCGGGCGGCATGTGGTGGATGGCATCAGCCGTGAGCACTCCAGCTGGGACCTGGTGGGCCTGGAGAAGTGGACGGAGTAC
CGGGTGTGGGTGCGGGCACACACAGACGTGGGCCCCGGCCCCGAGAGCAGCCCGGTGCTGGTGCGCACCGATGAGGACGGTAGGCAG
TGCCACCGGGGCGGGAGGGGAGGCGTTCTGCCTCAGACACCACCCACCAAGCTCCCCAGGGCCTTCCTTTCCTGAACACAGGCCCAG
GTCAACTCATCTTTCTGGTTCAGGTGTAATGGCCTAAAGTGGGGGGATGTCACTTACGGGATAACTGAGGCCCAAATCCCAGCCTTT
GGGGCTGTCTCCAAAGCAGCTGAAACCTTCACAGGCTAAGATGGGAGAAGCAGCCCTGTCTAAGATTTGAAAGGTCAAGATTGGGCA
GATTGGTGTAAAAGATACTCAAAGTAGAATCAGCAAGACTCCACGTTGGCTGGACATTGGCATGATTGGGGCCTAGGAGGAGTCAGG
ACAGGTTGTGCTGACTAGGGGTGGTCAAAGACCTGGTGCCCCACCTCCACCTGTTCCCCCATGTCGACCCTCCCCACCAAGTGAGAG
GCCTGGGCCAGAGGGGTGGGCAGGGCCAGTCCTGGGCTCTTACCCGTGGCGGGCAGAGGGAGCCTTCCGTGTGCCTCACCAGGGACA
GATGATCTAAGGAAACTGTATCAGGGCCTTTCCTGGGGGAGTGGGGTGCTGAGGCAGGACCCTCAAGTTTGCTGTGCCCACCTGAGC
TAGGGTTGATACCTCCAGGCCTGACTTCCTTCTCTACCTGACCCCCCAGTGCCCAGCGGGCCTCCGCGGAAGGTGGAGGTGGAGCCA
CTGAACTCCACTGCTGTGCATGTCTACTGGAAGCTGCCTGTCCCCAGCAAGCAGCATGGCCAGATCCGCGGCTACCAGGTCACCTAC
GTGCGGCTGGAGAATGGCGAGCCCCGTGGACTCCCCATCATCCAAGACGTCATGCTAGCCGAGGCCCAGGTGCAGCATTGGGTGGTG
GTGGGGTGGCAGGGTGAGCACAGACCAGCATGCACAAGCTCCCTTTTGGGGCCCAGATATGTCCCTCTTCCCCTGCCTGCCCTCAGC
AGTGCTGTGACTGCCTTTCCTTGGTTGTGAGACCCGAGATGCTTTGCAGCATCAGGGGTTAGGCTGGGGTTTTTTGGGTGTGGGTTT
TTTGTTTGTTTGTTTGTTTTGAGATAGAGCTTCACTCTTGTCGCCTAGGCTGGAGTGCCTCCTGGGTTCAAGCAATTCTCCTTCCTC
AGCCTCCCGAGTAGCTGGGATTACAGGCGTCTGCCACTGCGCCTAGCCAATTTTCGTATTTTTAGTAGAGACAGGGTTTCACCATAT
TGGCCAGGCTGGTCTCGAACTCCTGACCTCAGGTGATCCGCCTGCCTCAGTCTCCCAGAGTGCTGAGAATACAGGTGTTAGCCACTG
TGCCCCACCAGGTCGGGGTTTTGAGATGTGCCTTTCCCCTAGACAGTGCTGGGCTGGCATCCACTCTTCCCACAGAAAGGGTAGAGA
GAGTGCTCCAGGGCTGTCTTACCCCACTGGCGGCCATGGGTCCGTGGTTGCTGCAAAGCTCTGTGAGTAGCCAAGTAGAGTGTTGCC
CTGCTCCTGGCCCTGCAGGGAACGATTCAGCCCCTGATGTTCACCCTCAAGAGCTAGGCCTGCTGGCCAGCCTCACACCTCCCTCTG
TGCACATCTGTCTTCCTGGGAGGATGGTCCTGCCCTTGAGCTTGGGGTGAGGTCCCTGGGGTATTCTGAACACTGGTTGCTATTCAG
ATGAAGAACTTGGAATGCTGGGGGGTTATGAGAGTGGTATGGAATTATTCAGCAAGTAGGTGGCTGCTGCGCTTGGATGAGAAGCCA
TCTCTGTGGACCCCTAGGAAAGGGCCACAGTTGCTGTCATGAGCCCCCTCCCAAAAGACCCTGCTGGAGAGTCACAACACCTGGTGT
CGTGCTCTCAAGGTCTCCCTATCCAGGAGCAGGGCCTCCCCATTGAGCCTCTCACCTCTGCCTGGGTGGAGAGCAGGGGTGCGTGTA
CCACTCAATGCTGTACACACTGTGCAGAGGGGTGGGGTCACCCACACAGACAGGAGCCTTATTCCTCCAGCTGGGCTCAGCCATCTG
AAGCAAAATTCTTTCTGCCCAGAGGGTGCTCTCTTCCCCCTCTCAGCCTGCCCAGTGCTAAGGCATCCGGGGTGGAGGAGGCAGGCA
TGTGCACCCACCTGCATTCCTGGGGCAGGTTGAGGGCTCCTTGTGGAGCCTCAGTGAACACACCTACCCAGAAACATCCCAGGAATG
GGTTCCCCTAGCCCCTCCTCTCTGAGGGGGCCAGTGGCCACAGCTGTGGCCAGTGGGGTTCCAGAGAAGCCACTTCAAGTGCCCCCT
TCTGGTCCCAAGAGGTTCGGAAGGGAGCTGGGCCAGAGGCTCAGGGACGCTGCCCATTTAGTCTCAGACATCCCATCATGGGGGCGG
TAACTCGAGTCTGGGCTCCCGGAGGACACTGAAGATGGAGGCCTGCTCCGTAGCCCTCTCAGGACTGGGTCATTCTGTTCCTCGCCA
GTGGGAGAGCAGTGGGGCCTGGTCCCGAGCGTGGCCACAGGGCCCAGCTGTCCCTGTGCTTCTGTCAAGGGCGGCACATTCTGTCCT
TCACGCAACAGCACCATCCCCTCAGCTCATCCCCCATCATGCCTGAAGGAACTATGTGGTGCAGGTTCCCACCCCCAGCCTGGAAGT
GTGGAGCCAGACCTGTCTCCTTTTACGTCAGATTCCATCTCCCTGCCTCCCTTTTTCCCCTCTCGTGGCCTGCATTTCTCTCTCCTT
CTTGGAGTCTCTGTTTTTGTCTTGCTCTTTCTCTTCTCTCTCCTCCTTCCTTCCCTCCTCCCCCTGCCATTCCTCTGACCCCTTTCC
TTCATCTCCATTTGGTTTCATCCTCCGTCCTTCCCTTCTTTCTTGCTCTGCCTCTAGCGCAGGACAGGGTCCATGTGATGTGAGAAC
CTCCACGCCAAGGCTTGGTTGGGACAGCCCAGGTCTCCCCGCAGGCAAGGAGACTGGAAGGGACGTGGGCCCAGCCACACCCTATAA
AGTGGCCATGCACTAAGGACCTGTCCAGAGTCTCCTCTCATATTCTGTTCCATGCTTCTTGCAAGGACTTTCCAACAGAGTGTCCCA
GGACAGGAGGAACTTAGGCCACCCAGCATGGAGGCCAGTGGACAGAGGCCAGACCGGCACTGTGGGGGTGCTTGGGCTGGAGGACCC
CAGGTACTTCCGGCTTGGAGACATCCTGGACCATCTCCCTGTGATGTTTCATGGGGCCTCAGAATGGAGACCTCACAGTCCCTCCAC
CTGTCCATCTAGAATATCTACATGCACCAAAGGCCCGCAACACTGCCAGCCCCGAAACATTCCTTCCTACCTTAATCCTCAGACACC
CCGAGGAAGGAACGGGTATAGGTGATTTCCCGTGCCTGTGGGCTACAGCCCAGGCTCTGAGCTTGGCACTCACAGCCTTCATAGTCT
TGCTGACTGGCTTGCCCTTGGGCCCACCCTGGCCATGTGCCCTGTTCACCCCCAACTCTTACTGTTGTGGGACACTTCTCATGCCTG
GGGTGATTGTGTAATGTATGATCACATCTGGGATGCTTTGGCGGGTGGAAGGAGACACTGAATAGTCAATTCCATGGCAACAAGCAC
AGGCCAGGACTGTCCGAGGCAAACCAGGGCATATGGGCACCCCATTCACCCCTCAGTGTCTTCCTTTCCCTTGGCCTGAGCTGTTGA
ACCCACCCTTCTTGTCTGGCAGCCTCTCCCTCCCAGCAAGCCCAAAATTGTCAGAGCATCTGTAGCTGCTTTGTGTTCTTAGGGCCT
TCTGTCAGCGGGTCCTCTCCCTGCAGGCTGCCTGCCCTCCCTGTCTCCCTACCCTCCTCAGCCCTGGCACACCCAGTTGGTGCTCAG
TGAGGCAGGGATTCAAATCTTTGAACCGGGAGTTGTTCTGGGGTGCTACCCCCATGGGTACTTTGAGGCCCAAAAGCCCTCCCTCTG
TCCTCCCTGGGCAAGGTCCCTTCCAGGCCAGGCCCTCTCCAGGTCAGAGTCCTTCACCATCCTGTCTCCCTTTCTCTCCCTCTCCCG
CGGTCAGTGGCGGCCAGAGGAGTCCGAGGACTATGTAAGTAACAGGTGTGCGAACGCGGACAAGACATGGGTCAAGCTGGGCTCGTG
GGACGCTCCTCCTCTCCCTCCTTTCCTGCTAGCCTGCACTGCCAAGATCCACAGGGCTCTAGCCACATCAGGAGAAAATTGGCCTTT
AGACACAAGCACTGAGCTGAGCAGCGATTGGCATTTCCTCTAATCCTTACTTCTTGCCTGTCGAGCAGCAATTTGAGGCCAGTGTTC
ATGATCGACCAGGGCCTGGCCCCTGCCCCGGCCAGACAGGGATGGAGTTAAATCCAATCCTGATCGTTAGGCCTTATTGATCCCTGG
AGTGAAAAATCACCTGCTTTAGGGCCCAGGCTGGGAGGGCTGTCTGGAGAGTCGGATTCTGGCATTGGTGCATTCTGGAGCCCCAGC
CCTGGGAGACCCTCCAGCTGTGGCAGGAGGGGTCCATGAGGGGGTGGTGGCAGCTGCAGGGGCCCCACTCAAGGCCAGAGCTGGAGG
GATACCAGGGTTGATGACGGCTCTGTTCCCACTGCACGGTGGTCCCTGCCCTGCTTCCCACTCTTCTCTCTGTGTGGTGTGGCTTGA
CCTCCCATGGTGTTTCTCCGCATGTCCAGAGATGATGCCTTTGCTGCAGATGGGTATATGGGCAGGGTCTGCCAAGCGGGGAGGACA
TTGCCCTGGCTGCTGTCTCAGGCATCACTGAGAACAGATGTGGAAGCCAGTTCCCCAGGTGTGGAGGTTTTCTTATTCTCCTAGCCC
CTCCCCTGCCTTTCTCAAGAGGTATTGCAGGGTATAGACATTCACAGAGTTAGTGGCCTATATGGGGGCAGCGGAGTCCTGACTGGG
TCCTATAGGATGGCACCTTAGCCCATCCTGCCACCTTGCTCTGTCTGTGCATGCATGCACTGGTGTCCACAGGCAGCCTTACGCCTG
CTTATGCAGGAGCTGTAGGCTGTGTGCGTGTGGCTGCCAAGAGCCACGCAAGGTGCTGGGTGCGTGCGAGGCTGTGCCCTGTTGATA
TCCTCAGTCTCCGTTCACAGCACAGTGGAGTGAGGGAAACAGTCTGGCCCTTTGTTCTTGTCTGAAAAGAATAATGAGCTGTCTGCC
CCAGGCGTGCGGCTCCTGGGATGGGCGGGGTCCTCCCAGGCCTGCATCCTACCTGCCTGCTTCCTCTCCAGCAGAGGCCACCATTGT
ATAGCCCCACCTTCCACAACCCCTGGCCTTGTGTGCCCCGGGGCTCCCCTCAGGCTAGGGTCCTGAGGTCCCTGACAAGGTCTGGCC
TCTCCCTGCATTCTTGTGATGGGAACGAACCCCTCCTCCTCCCTCAGGAAACCACTATCAGCGGCCTGACCCCGGAGACCACCTACT
CCGTTACTGTTGCTGCCTATACCACCAAGGGGGATGGTGCCCGCAGCAAGCCCAAAATTGTCACTACAACAGGTGCAGGTGAGTGAG
GGGTCAGGACGGACCTGAGGGTGGGGCAGCAGGAGGGCAGCGCCAGAGCCCAGCCCGTGGTCCTTCAGTCCCAGGCCGGCCCACCAT
GATGATCAGCACCACGGCCATGAACACTGCGCTGCTCCAGTGGCACCCACCCAAGGAACTGCCTGGCGAGCTGCTGGGCTACCGGCT
GCAGTACTGCCGGGCCGACGAGGCGCGGCCCAACACCATAGATTTCGGCAAGGATGACCAGCACTTCACAGTCACCGGCCTGCACAA
GGGGACCACCTACATCTTCCGGCTTGCTGCCAAGAACCGGGCTGGCTTGGGTGAGGAGTTCGAGAAGGAGATCAGGACCCCCGAGGA
CCTGCCCAGCGGCTTCCCCCAAAACCTGCATGTGACAGGACTGACCACGTCTACCACAGAACTGGCCTGGGACCCGCCAGTGCTGGC
GGAGAGGAACGGGCGCATCATCAGCTACACCGTGGTGTTCCGAGACATCAACAGCCAACAGGAGCTGCAGAACATCACGACAGACAC
CCGCTTTACCCTTACTGGCCTCAAGCCAGACACCACTTACGACATCAAGGTCCGCGCATGGACCAGCAAAGGCTCTGGCCCACTCAG
CCCCAGCATCCAGTCCCGGACCATGCCGGTGGAGCAAGGTGTGTGCTGTGGACATGGCATCCCTTCCCGAGTGTGGCTGCATCTGGG
GGTCTCTGCTCTCCTTGAGCCACTGACCTCTGGCGACTGTGATCCACCAGCCTCTGGTGTGTGACCTCCAATCTCTCATGACTGTGA
CCACTAACCTCTAGTGAATGGGCACCACATTCTTGATGCCTGACCTCTGCTGTCCTTAACCTACTGACCTCTGCTGTATGACCTTCT
GATCTCTTGTGACCTTGACCCACTGATCTCTTTTGACTGTGTCACTATTCTTGGGTGTGCAACCTCCTGATCTTTGGTGTGTGACAC
TAATCTCTTGGGGCCATGACCCACCGACCTCTAGTGAACATGCTCCACCACGCTCTGGTGTGTGGCCACATGCTTCTCATGACCGAA
ACCCACTGACCCTCTGATCACTCTGGCCTGGTGTCCATCGGCTCAAGCTTTTACACTCGCGTTTCTGGAGATTCTGACCCTGGTTGC
TGTGGCATCCCCCGCCCTGTTTGGTGCTCACTGTGGAAGAGCTTCGGCTGGGAGTTCAACTGTGCCGTTTGAAGCTGGCTGGGAGTG
GGGCGCTTGGTACTCTGCAGCCATCCTTCAACCCCCTGTTCCCCAACCAGTGTCTCATCCTGGCCATTCACCTTCCACCCTTCCACC
CCATTCACCCCACCTCATATACCCAGCAGAGCTGACTCTCTCTATGCCTTTGCAGTGTTTGCCAAGAACTTCCGGGTGGCGGCTGCA
ATGAAGACGTCTGTGCTGCTCAGCTGGGAGGTTCCCGACTCCTATAAGTCAGCTGTGCCCTTTAAGGTGAGTAAGGGCCACGGCCAG
CTGAGCCTGGCACAACACAGGCCTGCTGGGTGCTGTCTTTCCAGTCCTAACCCATGTGCATCCGGCTGTGGAGCAGGAATGTGGTTT
GTGTATCCGTGCACTGTGCCTTGCAGCCCGTGGTAGGGAACCTCACCCAAAGGCATTGATTGCCCCTCCCGTCCCCCACAGATTCTG
TACAATGGGCAGAGTGTGGAGGTGGACGGGCACTCGATGCGGAAGCTGATCGCAGACCTGCAGCCCAACACAGAGTACTCGTTTGTG
CTGATGAACCGTGGCAGCAGCGCAGGGGGCCTGCAGCACCTGGTGTCCATCCGCACAGCCCCCGACCTCCTGCCTCACAAGCCGCTG
CCTGCCTCTGCCTACATAGAGGACGGCCGCTTCGATCTCTCCATGCCCCATGTGCAAGACCCCTCGCTTGTCAGGTGTGCACACGAG
GTATCGGGGGAGGCGGGGCAGGGCTGGAGGTAACCAGCAGTGACAGTCCTGATTCCTGCCCTGCCCACCCAGGTGGTTCTACATTGT
TGTGGTACCCATTGACCGTGTGGGCGGGAGCATGCTGACGCCAAGGTGGAGCACACCCGAGGAACTGGAGCTGGACGAGGTACCTGG
GGAGGGGATGGGGACACTGACAGCCCCATTGCAGTGGTCAGCTGTGGCCTTCCTGCCCTGAGCACTGTCCCAGTGACTCTCAGATTC
ACTCCCCAAATTGAAATCTCTCTTCTGGCTGGCAGCCCGCCCCTCTCTGGAGAGAGGGACTCTGAGGAAACCATCTGGGAGTATTCA
CAGAACTCCCGGAGGGCTTACGAAGAATCCCTGGGGTGGGATGTCGTGGAGATGCCTCTGCAGGTCTAGAGAATGCCAAGCCCTGTA
GACACTGCAGAGCCTCGCAGATCTAGAAGCTATGGAGGGCTTAGTGAGCCTTATAGCCAGGAGTCCCTGAATGTTTGAAATCCTTCA
GTCCTTTCTCATAGCCAGTGTGGCAACAGCCTGGTTAGGGTGGGGCAGCTTACACACAGGGCTGCTTCTCATGGGCTGGTGGGGAGG
AGTGGCTTCACGGTGTCTGTTACTCTGTAGGGGCAGTGGGTTGGGCAGGTGTGGGCTCTTACACGGAAGGTGAGCCTTGATCTCGGC
CCAGGGAGCTGACATCCCAGGCCACAGCCCCAGGGCTGGCCGGCATGCTCCAAGGCCCCTCATGACCCCCATGCTCTGCTCTGCCAG
CTTCTAGAAGCCATCGAGCAAGGCGGAGAGGAGCAGCGGCGGCGGCGGCGGCAGGCAGAACGTCTGAAGCCATATGTGGCTGCTCAA
CTGGATGTGCTCCCGGAGACCTTTACCTTGGGGGACAAGAAGAACTACCGGGGCTTCTACAACCGGCCCCTGTCTCCGGACTTGAGC
TACCAGTGCTTTGTGCTTGCCTCCTTGAAGGAACCCATGGACCAGGTCTGCCTGAGCCGGCTTGGCTGTCAGCACCCTGATTCCCTG
GGCCTGGCCTGAGACCATGCCAGTCTCAAACACCACAAGACCCCAGGTCTTTATCAGTTTGGGGGCTTCGAGATCCTGGGGCAGCAC
TAAAGACCCAAGATCTGTCCCGGGGATCCTAAGACGCGGCCCTGGGACCCAGAGGCCAGACCTAATGTGGCTCCAGGGACCCAGTCC
TGCCAGGTCCACCTTGTAGGGTCTGGGAGACCAGGCCCAGGGTAACCCAGACCCAGAGCCCTTTCTCCAGGATTGATAGGCAGAGGG
TGGGGGGTTCTCACGCTGAGCTCACAGCCTGCTGTTCTCCACCGGGCCACAGAAGCGCTATGCCTCCAGCCCCTACTCGGATGAGAT
CGTGGTCCAGGTGACACCAGCCCAGCAGCAGGAGGAGCCGGAGATGCTGTGGGTGACGGGTCCCGTGCTGGCAGTCATCCTCATCAT
CCTCATTGTCATCGCCATCCTCTTGTTCAAAAGGTGAGCACTGCCCTCAGAGCTCCGGGAACGGCCACCTGCCCCTCGCCTTTCAGG
CCCTCTCCGGGTGTGGTGCCTGTGGAGAGCGTGCAGCCTTGCATCCTGGACCCTGAGCCTCAGGCTCAGCAGGAAGGCAGGAAGGGC
AGGAGCAGTGTGTGTGCCTACCTGTGTCTGCAGGCCTGCATCTGTCAGGTGAGGGAGCCTCTGCAGCAGCCTGGGCGTGAGGAACTA
AAGCCTCAGAGGGTTGTCTCATGTGCATCCCTGGCACGTCTGTCTGTCTCTGTGTTTCTGTGGATAAGAGTGCGTCTGTCTCCTCTT
CAGTTTTAAGACACCGCCATCTCCTGAGCTCCTACCTCGAGTCCTTTCCTTCTGCCACCATTCCTGGATGATTCCCCAGCCCTACTC
TCCTGCTTAGGCCTAGAGAGGTCGAGCTGGAGGCCCACAGGCCATGGATGCTCACTAACAGACATGACTGATGGCCTGCTGTGCCCG
TGCCATCCGCAGCGGCCTCAGCCTCAGTGTGTCTTTCCCACTCAGTTCTCTATCGGCAGTGGCCACCACCTGCCCTAGGAGTCTCCC
TTCCTATCCGTGCACCTTCCAGCCTGAAAGGGATTTCCAAAGATTTAACTTTGTCACTCTCAGTTTAAAACCTTTTGATGGCTGCCC
ACGTAAAGCCCACCCTCTTTAGCCTGACAGCCAAACACCTGGCTAATCTGACCCCTGCCACACTCCTGACATTGCGACACTGAACTC
ATTCAGTGTTCAGGCCCACACAGGGTGTGCCCTCTGCGGATCTCCCTCCCCCTGCCCACTTGGCAACAGCAATCTCATCGTTCAGGA
CTCAAGTCCATGCTCTGGTGCCTGCAAGGCCTTGCCTGGCCCCTACCCCTTCCTTGCACAGCTGTCCCCTGTGATGCTCCACTGCAA
ACGCAGCTGGGCCTGTGTGCCAAGCCTTCAGCCCCCAGCACTGGGCTGAGCCCACAGGCATGTCAGTGAGCACCTGGCAGATGAAGA
TATGCTAGTCTGTGTGTCTGAGCACATCTCTGCACCATGCAGGAGAGCCTGTATCCTGTGTGCCTACCCTGGACCCACTGTTCCTTC
CTAAAGAGTCCACTCCTTCCTCCCAGGGTGGATGGACCTGTGGGCGGGTCCCTAAGGAGTGTCCCACAGATGCACTCATCCTTCCCA
CTTCCACCTGCATTCTGGGTCTGTCCAGAGAGGTAGACCCCCTCCCTGTGAATGTCATGTGGGTGCCATATAAGGGGCCTGCAGGGT
CCCCATGTCATTAGAGGGCTGAGTGCTTGGTGGAAACTGTGACCGTTCCTTTCTTCTGTCCTTGCTTGTCTTTCGGGACCTAGTAAA
CAAGAAAGGTAGGAGTTGTTCCTTCTCTCTTCACACACCTCCCCCTCTACTTCTAATCCTTGGGCCAGCAGGGAGGGATGACGCTTT
GACCAGAAGTCCCCTGGCCTATGGCTTTAAGCAGCAAACTGTCCAGTCAGCTCAGCAGGCTCCCTGGTTCAGGACTGAGTCCTCATT
GGTGTGAGGAGGCAGTTTCTGTCTCACTCTTGCCAGTTCAACTCTGCAGAGGATGCTTCTTCTCTGGGGCACATACATAATACAGAC
TGCATTGCTTTCCCTGAGCGTACAGAGCTCACCAGAACCATCACCCGGCTCCCAGTTCTCTGAGAAGTTGCCTAAGTGCTGCGAGAG
ACAGTCTCGCACAGAGCAAAGAGCAGTCACTGTGTGGAACAGCAGAGAGGCATGTCCTTGTACCCACCTGAGCACACTCACTCAAAT
TCTTCTTCCCGAAGCACATGCGTCTGCAGACCACAAGTAGCCTCTGGCTATTTGTTGGCAGCTGAATCCTATGGTCTGTCTCTGACC
CTGTCCTTGTAGATGGGAAAACTGAGGCCAGTGAGAGAGGACTCTGCCCAGCTAGGGTGACGCCTTTCCCGTCCCTCCCCTGCTGCC
CTGGGCATTTACCCTGATACTCTGAGGGCCAAAAAGCTGGGGCCCTGTCTCCTGTGCTGTTTGTGTATTTTATGTGTCATTTGCATG
TTTGTTCCCGGTGGTTCCTGCTACTCAGCTGGGTGGCCTTGGAGATAGCAGTCACTGTCCTGCAGTGACCTGCCTTCTATCCAAAGG
AGCTGCTCGCCCCTGCAACCCCTTCCTCCCTGCTGCACTGCCCCTCTGCTCACGCCACACTCCTGCTCTCCCTACCCCCTCCCTGCC
CTGTGTTCTCTCATGTCAAGGCTGTTCTGGCCCTGGCTCCTCCGCGCTCAGAGATCCTGGGAAGAGGTGGGGCAGTAGGAGGACAGA
GCCGTGGACATGGGGGCTCCTTGGCAGCCAGCCATGCCATGTGTCACTGTCTCAGCCTGGACCTCAGGTTCTCACCAGCCTCCCTTC
TGTCTCTCTAGGAAAAGGACCCACTCTCCGTCCTCTAAGGATGAGCAGTCGATCGGACTGAAGGACTCCTTGCTGGCCCACTCCTCT
GACCCTGTGGAGATGCGGAGGCTCAACTACCAGACCCCAGGTAGGGCACTCCTATGGCCTGTGTGCCCCCAGCCCAGACCTAGCAGG
CCTGTGGGTCCGTTCTGCAGGTCTCAGGGTCGCCACTGAAGTTCCTGGGCCGCATGCACTTGTTCCTGCTCCTTTTCTTCCTTTCTG
CCCTTCTCCCTGTGCTCATCCACTTTGGTTGGAATGACTGGGGAGCCCCCATATCCTGCAGCTGGACAGGTGCCCCAGGCCAGGACC
CTCTTAGGTGGCAAAGCCACTTAGCCACTGCCTTGTCTTCAGTCAAGGCCTCTGAATATGGGCTGAGGACCTCCCAGAGCCCACGTC
TCTGCCACAGGGTGGCCAGTGTCTGGCCCACGCTCTGACGCCTCCACATGAAAGAGGGGCATGTTAGCAGTGGGAGATGCCTCACCA
GACATCTGTGGGGAGATGAAGTGCCTGCACCTGAGCCTTCCTGGAGGCCGACCCCAGGTTTACCATACCACGTACCTGCCCCTTCTC
CTAGCCACTCTCATGGGGCTGTCCACCTCTTCCTTCTACCTGAAATGTTACTCCTCCCAACACCCAGCAGACACAGCTCAGACCTCT
CCTCTTGCCCACACCCCCACAGGGTTAGGTGTCTCTTCTTGGCTCCAGGAACCTCCCTCCTTCTTCCTTTTGACGCCTCTCACAGTG
AGCTGGGATTACCTCAGTGACAGTCTCTGAGCTCTGTGGTGCCAGGAACTGTGTCTTGTCATCCCCCATCCCTTGTATTCAGCGCAC
GATGGGTTCTTGTTGAATGCCTGTGAAATGAACACACAGACAAATGAAGGAATGAGTGAACTAATGCATACACACAGGGTTTGTGTA
TGTATGAGCTGGTTATTCTGCATGTCAAATTACCCCAAACTTAGTGGCTTAAAACAACAATAATTATGTATTATCTGTCACAGTTTT
TTGTGAGGCAGGAATTCAGACAGGGGCACAGCAGGAACAGCTTGTCTTTATTCCAGAAAGTCTGGGCCCTCAGCCGGAAGACTTGAA
GGCTGGGAGCTAGAGTCATTTAAAGCAGGGGTTGGCAGACTATGGCCCGTGGCCAAGTACAGCCCACCACCTGTTTTTGTAAATAAA
TTTTTACTGGCTGGGCGTGGTGGCTCATGCCTGTGATCCCAGCACTTTGGGAGGCTGAGCCGGGTGGATCACCTGAGGTCAGGAGTT
CGAGACCAGCTAATGAAATCCCATCTCTACTAAAAGTACAAAAATTAGCCAGGTGTGTGGCCCCTGTCATCCCAGCTACTCAGGAGG
CTGAGGCAGGAGAATCACTTGAACCCAGGAGGTAAAGGTTGCGTGAGCCAAGATCGTACCATCGCATTCCAGCCTGGGTGACAAGAG
CAAAACCCCATCTCAAAAATAAACAAATAAATAAAATTCTACTGGAACAGACACACCCATTTATTTATATGCTACCTACCTATGGTT
GCTTTTGCACTGTAAAGGCACCATTAAATAGATGCAGTAGATACCAGATGTCCACAAAGCCTAAACTATTTACGCTCTGGCCTGTTA
TGGATACCAAGTTTGCCAACCCCTGGACTCTAAAGCCTCTTTCATCCATGTGACTGGTGGTTGATGAGCCAGCTCTGAGCCTTGCTG
TGGCTGTCAGCTGGGACACCCAACATGAGGCTTCTTCAGGTGGCCTGGGCTTCCTCACAACATGGTGACTGAGTTTCATGAGCATGT
GTCTGGAGAGTGTGCCAGGCAGACCTAGCAGGCTGTTGCAACCCAGCATGATTAGATCTCTGAAGAATCAAGCACCAGGATGCTGGG
ACCTGGAGTGCAGAGAAGCCTCTTCGGGGATGGGATGCTGAGCCATGTGGTTATTGGTCTGGAAAAGAAGGATGGGAGAGAGCTCCT
CAGTGGAAGCATGTGCAGAAGCTTGGGCAGATTGCATGTGCGTCAAATTATGAGCACCTGAGGGTTTCTGCAGCATGAGAGTTAGGG
GCTTGGGGAATACTGAGCCAAGAGAAGACTGGAGGGCTAGGCAGAGGTCCAGTCAAAAGGACCTTGAATGTCAGGCTAAGGGGCTTA
GACTTCCTCTTCAGGATGCCAGGGAGGTAGGTATTAAAGGTTTTCAGCGGGGGAGCCAGGTGGTCAGAACCATGCTTTAGAAATGTC
ACTCTGGCTCCTTGAGGAGACTGGATTAGAGAGGAGAAATTGAGGCAGGGAGACCATTTAAAAGGCCAGCAACCAAGTGAAAGGTGG
CAGTGGGAACAGAGGGGACAGACTTGGGAAACGTGAACGAGCTGAGGACAGCAGAGTCCGGGCTTAAGCCCACTTGGGGAGAGGTAA
GGGTGGCTCTGCAGTTGCTGGCTGAATGAGCAGGGGGTAGGGGTGTCCTGGGGCTGGTGGGTCTGTCGGTCTGAGGACTTCTGAGCT
GACATCAGGGCTGAAGCCCTGTGGGAGGGGCCAAGGAGCATGTGGTAGTCAAAGCCTGAGAATAGATGGGCTCTCCCAGGGTGAGGG
CCAGAAGGAGACCGGGACCAAAAAGCCCAGGGCAGGGAAGGTGGTGCAGCATTAAGGAAAGATGGATGGAGAGCACCTAGGGAGATT
GAGAAGGAGCAGCCACAGCCAGACGAGAGCAGGAGCGCAGTGTCCCTGATGCCAGGGTTGGGGGAGTTGTGCAAGGGACGAGAGGAC
TTAATGAGGTAGGGCAGTGGGGACACCCAGTGGGAGGACCGTTGGCTTTGGCAGATGGGGCCTTGAAGAGAGGGTGGTGGCCACGTC
CTGTGGGCAGAAGTGTTGCAGTGAGCAGCGTGGCAAGTGGGGGCTGAGAAAGTGGAGAAACCCCTTTCAAAGATCCTGGGAAGATAC
CTGGGAAGGAGGCAATGAGCAGGGCACAGTCGGAGAGTAGAGGGTGTGGGATCTGAGAAGGAGGGCTCTTTTTTCCAACGTGGAAAT
CATCTGAAAATCGCCAAGGGCAAATTTTGGTATCAAAAGGGGCACGGCTGGTTTGGACTTAAGTATTTAGTCATAGAGCCCCCCAAG
GCTGCCGAGCCACCAGGGTTTAGAAGTGCTCGCCTCTGGGGGTGGGACACCCAGTCTGTATTAACTGGGAGACAGAAGGAGCCTTGA
CGGAACTTGTCCGCAGCCCCAGCCCCTCACCGCCCCCTCCTCTTCTCCGCCAGCCCCTCGCAAGCCCGCTCGGCACCTCCACTGTGT
GTGATGGTGCTGTAAGCAGAAAAAGTTAACGGGCTCTCTTTTCTTGCCCCGTTGTTTTTTTCGTTTGTTTGTTTGTTTTTTTCTCTG
CAGGTTCCAGTGTCCCCAGTTGCCCGAATACCTCAAGTTAGTTTTCAAAGTTCCCGTGTTTGGGGGATACTTTGGCTTCTGTGTCTG
TTTCCACTCCTTACTTTTGTTTACCCCATCGCCTCCATCCTTCCTTGAATTCTTCTCTCCCCTTCCCTTTCTTCTCCCCCAATCCCA
CTGTCTCCTAACCTTTTTCCTTCCCTACCCTCCCCTCTGCCCACCTTGCTTCCTCCAGGCCTGTTTTCTCTCCCACCGGCCCCGTCT
CTGTTCTGCCTCTCTGGCCTCCAGTCCCGGCCTGACACCCTTCCTTCTGCGTGCCTCTACCTCTCATCTCCTCTCCTCTGTCTCACA
CCCCCCTCCTGGTCTCTGCTTCTCTCTCTATTGTGTCTGTACTTCATGACCACTCCATCTACACACTTGGTGCCCGGGATGACGATG
GCTGTGAGTCTCCCATGGTGACTGCCACCGGTGAGGGGCAGGAGGGCTGTCTGCAGGCAGATGCGATGGAGCCCAGCTCCTGTCACG
TCTGCTGCCACCGACCTGGGCGTCCCACCCCTCCTGGGAGGAAGGAAGCCTCTCTTCCATCTTGAGAGACCTGCCAGGCAGGGCCTA
GTGCCCCCACTCAGCACCCCGCCACCAAAACAGGCTCCACATGCTCATGGCACAACACCGCCCTCTGTCCTCTCCCACCCTCCGCCA
TCCCTGTCGCCGCATGTGCTGCTGTCTCCATGCCACCAGTTCCAAGTGCTCCATGGTCACACATGTTCACATGTGCACATACATGCG
TTGGGGCTTTCTCTGCCACACTGCTCAAGCCTCACACTAATGCTGCCTGTGTATGCCCTACCTCCCCTAGGTATGCGAGACCACCCA
CCCATCCCCATCACCGACCTGGCGGACAACATCGAGCGCCTCAAAGCCAACGATGGCCTCAAGTTCTCCCAGGAGTATGAGGTGAGA
TGTTCCCGCCCCCTACCATGTGCCTGGCCCAGGCCTACCCAAACCAGCTCCTGTCCTGTCCTAGGTCCCAGCTGTGGTGGGTGAGGA
AGCAGGGGTCCAGCTTTTTCAGGAGCACAGAGAGGAGGGTTGGCAGGGGTAAGGGTCAGCTGGGAACCGGGTGCCTCAGATGCTGGG
TCTGGCCATAGCCTGGCCCAGCACCTTCTTGGGGTCACCCTTAGGAGATGGTTTCAAAAGGCTGTGAGTGACACCAGGGTCTGGACA
CTCAGACACGTGCTCAAGTGCTCACAGGCAGACACAAGGCCACAGGCATACAGACATAGATCAGTGATAACAGCCACAGTAGCTGGC
ATCTATCAAGAGCATCCTGCAGGCCAGCACCGGTCTGGGCACTTGGCATGCATTATCTCTTTTGGTTCCCCCACAGTCTTCAGAAAT
GGGGACTGTTATTATCCCATCTTCCAGGTGAGGAAGCGGGGGCTCAGAGAGGGGAAGTGACTTGCCCAAGTCAAACAGCTGATGAGT
AGTGGAGCCAGGATTCAAACCCAGGCCTGCCTGCTGCCCTGTGCTCTGCACATGCATGTGCTCACGTGTGTACTCCGATGCCACAGC
TCACGGGGAGTCGGGGCCTCGAGACTGGCTGCTCAGGCTGTACAAGTCCCGCTTGGAGCCCTCCACACGTTCATCTTGTTCTGGACT
TAACTCCTAGGAGCCTGCTGGGGGCTGAGCCTTCAAGAGTCTGAGGGTTTCCCACCCACAGATGGTGTCGGGGTGACTCTGAGCATC
CCCAGGCTGCCCATCTAGGAAGGGGATTTGTTAGAGAAGGAGGTGATTTAAAGACAAGACTCCTGGCCAGGCGCAGTCGCTCACGCC
TGTAATCCCAGCACTTTGGGAGGCCGAGGCGGGTGGATCACCTGAGGTCGAGAGTTTGAGACCAGCCTGGCCACCGTGGCGAAACTG
CATCTCTACTATAAATACAAAAATTAGCCAGCTGTGGTGGCACATGCCTGTAGTCCCAGCTATTTGTGAGGGCTGAGGCAGGAGAAT
CGTTTGAACCCGGGAGGCGGAGGTTGCGTTGAGCCAAGGTCATGCCATTGCGCTCCAGCCTGGGTGACAGAGTGAGACTCCCTCTCA
AAAAATATAAAATAAAAAAAACTCTTAAAAAAGAGTATTTTTACTTAAAAAAGAGAGAGAGAGACCTCCTCCTCTTCCACCTCCTCC
AAGCAGCAGCCTGTGCTTGTCGTTCTGCCTTGTCCACAGCTGTTTCCTCAGCACCTGGCACTGGCCTCAGTAGATGGTTGGTGGACA
GGCAATTGAGGGGTTGGCTGAGCCTAACCTGTGAGTTTGCGCCCCTTCTGATGTCCACCCTCAGCTGTGTTTGGGGGATGCATCCTA
GGGCTCAAAGATTGCCTTTCCCAAGGGCTGTGGGCCAGGTTTCTGAAGAGAAAGCTGGGCTTGGCAGGCAAATGGATGAGTATGTCT
GCGGCACAGCAACCGTGCTGCCTCTGCCTATAGGGCCCCCCTGGGGCCCTGCTCCACACAAGGCTGGGCTTTGGGTCACGGAGCCCG
TAAGGTGGGCTCCCTGCCTCCCATGGCCTCCACCCACACTCATCTGTACCATGTCCTACACCTGCCCTTCCTTCCAGCGCAGCCCTG
CTTCCCCATCTGGGCTCGTGGGGCCTCTGTTCACAGTAGTCCCCTTCCATCTTCTACCTGCTTCCCTCCTCTGATCAGAGCTTTCCT
TACAAGACCCTCCTCCTCCAGGAAGCCTTCTCAGGCTCCCCAAGGCTGCCGTGGGCTCTCCCTCAGCCAGGACCCCATAGCTCTGTT
GTCTTTTTTTTTTTTTTTTTTTTTTTTTTTTTTCTTTTTTGGAAGTCTCGCTCCGTCATCCAGGCTGGAGTGCAGTGGCGTGATCTC
GGCTCAGTGCAAGCTCTGCCTCCTGGGTTCACGCCATTCTCCTGCCTCAGCCTCCCGAGTAGCTGGGACTACAGGTGCCCGCCACCA
CACCCGGCTAATTTTTTTTATTTTTAGTAGAGATGGGGTTTCACCGTGTTAGCCAGGATGGTCTCGATCTCCTGACCTCGTGATCCG
CCCACCTTGGCCTCCCAAGTGCTGGGATTACAGGCGTGAGCCACTGCACCAGGCCTGCTCTGTTGTCTTTAACGTTCAGTCAGTCAT
TGATCACACATTTTCCATGTGCCAGTCCCCGTGCTGGGGATGTGGAGACACATGCCCTCAGGGAACCCAGTCGTGGGGAGCATGTGC
AGACAGATAATCAGCATTGAATATGTGACTCTGACAGCATGCCCTGGTGTTGAATAGGACCGACAGGCGTGCAAAAGGTGGGCTGAC
AGCACCAGAGCAGCCTGAGGGGTGGGGTGTGCTGCGGGGTCTCACGGAAGCAGTGACGCGGTCCTGTGGAGAGTAATGAGGCTGGGG
ACAACATATGGAGGACATTGCGTGCTATGCTGAGAACTTCAGATCGTATCCATCTGGAAAATGTGAAGATAATAATGGAACCTATCT
CGTATGAGAATTTGAGGAGGGAATACATGCAAAGTGCTCACAGCGGTGCCAGGCACACTGTGGGTCCTCACTAACGCTTCGCTATTA
TCATTATTGCTCTCCGAGGTGAAGAAGAGCCACAGAAGGCCTTTAAGCAGGAAATTGATATTCACATCTGCATTTTGGAAGGATTAT
TCTGGCTGTGGGGCTGAGTGGGTCACAGTGGGACAAGAGGAAAGAGGCAGGAAGAGGCTGAGCCTCAATTGGGGTGGCCTGGCCCAG
GGCAGTGGCAGCAGGTTGGAGAGAGCTGAGGGGGTGGGGAAGCACCAGAACCTGGTGACTGGCAGGGCGGGGTTGGTGGAGGCCGGA
GGAATTTTGAGCTTGACTTCTAGGTTCTGGCCTGGCACTGAGGGGTGAGGGTGCCTTTCGCTAAGGTGTAGTCCCAGCAGCAGCGCC
AGGTTTGGAGGGGAAGCTGATGAACTCAGCTTTGAAAGGACTGCGGTTGAAGGACCTCCCGGTGGAGGTGTCAGAGAGGCTGCTGGG
TACTCGGGGAAGAGCTCTGTGCTCCTGCTGAGGTTATGGGCAGCTCCAGCATGTGGTGTAAGAATCAGGTGAGCAAATGAGGTCACC
CAGGAAAGGTTGTAGAGAGAGAAGAGGGCCTAGAAGGAACCCTGGAGGAGCTCTGCGTCCTGCCCACGGGAAGGGCAGGGGAGAGAG
AGGAAACAGAGAAGGAGCAGCTGAAAGGAAGAGAGCCGAGCAGGGAGGGATCCTGGACTACCTGAGAGGGGCCACCATCCCTTCCAT
CTGTCCTGGCTCATTTCATTAGGTGAATGCAGGGCTTCGGGAGGGACTCTGGGTGTTCAGAGCCCTCAGCAGTTTCGGGACTGCCTG
AGAGGGGGCCACCACCTGTCCTAGCTCATTTCATTAGGTGAGTGCAGGGCTTCGAGAGACCCTTCACCTGCCCCATCCCAGCTCAGA
CCACTCTACCAGGCAAGGGGATTTGGTACCATGGTCAGAGGAGTCCCCAGTCCATCACTTTTCTGATAGGTGATGTGGCAACCTGTG
AGCTCCATGGCTGGCACCACGAGATAGAGGGCCTGGCTGTGGCCTGTGGAGTGAAGCCAGGATGTGAGCATCACCGGGAAGGCTGGG
TCCCCTGCAGGAGAAGCAGGTCAACCTTGGCTCTTACCCCACCCCACCCGCTTTCTCCATTCTCTGCAGTCCATCGACCCTGGACAG
CAGTTCACGTGGGAGAATTCAAACCTGGAGGTGAACAAGCCCAAGAACCGCTATGCGAATGTCATCGCCTACGACCACTCTCGAGTC
ATCCTTACCTCTATCGATGGTGAGCCAAGGGGGTGCCCCTCCCATCCCCTTGCTCTCCCCCTTGCTAGCTAGGGCAACATGTCATTC
TACAGAGGATGTCCACGAGTCTCAGGGGTGCACTGAGGCATGGTGGGCTGGGCTGGGGACCCTGTAGTAATGCCCTCCCACCTCCTT
TCTTATCCATAGGCGTCCCCGGGAGTGACTACATCAATGCCAACTACATCGATGGCTACCGCAAGCAGAATGCCTACATCGCCACGC
AGGGCCCCCTGCCCGAGACCATGGGCGATTTCTGGAGAATGGTGTGGGAACAGCGCACGGCCACTGTGGTCATGATGACACGGCTGG
AGGAGAAGTCCCGGGTGAGGCTGCAGGGCCCTGCCAGGAGGCGGGTGGGAAATGCCCAGCCACAAGGTGATACAGGGCACCTTCTTC
TGTGCCGCTTTCTTCTGTGGACGAAGTCGCTCAAGTGATCCCCAGATGCTATTGTTACTGGGGGTATTATGCTCCCCAAATACTGGG
TGTTTCTGGGATACAGCATGTTCCCCACATGCTAGTGGGTTCCTTAAGATGTTAATATGTTTCACCAAATGCTGTCATTTTCGGAGA
ATGTTAATGTGTTCCCCAGTTGCTGGCGTGTTCCCGGGGTATTCGTGTGTTCCCCAGATGCTGGAGTGTTCCTGGGGCATTAACACA
TCTCCTAAGTTAATTAGTGGAAAGAGCTGCTGTTATCTGTTTTTGGACTGCAAAGCATTGACTGGAACGTAAAATTTACAGAGCTTG
AAAATGGCACATAAACGTTATGGGTAAATTTGGCAGAAAATGTGCCTGGTGCGATCTGGCGTTGAATAAATAATTTTCAATTATGAC
CCTTATTCCACTAGCTAGGGCAGGGCGGTGACATAGCTTGAGGACCTGATGTGGCTTGTGGAGCACAGGGGGCAATGGATCTGAGAG
CTCAGGGCTGGGGGGCTTTGTAGCCAGAAAGGCTACAGCCAGGGAGTTGACCAGCCTCCACCCTGCTTCTGCCCGTCTGAGCCTGTG
GGCTTCCTTCAGCCTGCCCTGCTCATCCTCCTGCAGGTAAAATGTGATCAGTACTGGCCAGCCCGTGGCACCGAGACCTGTGGCCTT
ATTCAGGTGACCCTGTTGGACACAGTGGAGCTGGCCACATACACTGTGCGCACCTTCGCACTCCACAAGGTATAGCCTTTCCCCAGT
GCATATCTCTTACCCAGACACTGTAAGGACAGTGGCCTGGGTGTGGTGTGCTGGGTCGGGGGGAAGCTGGAGCCTGGGTGTTGGAGG
GTCGGAGGCTCAGGTGTGTGAGTGATGTGATGATCCATGTTATGGGAACAGTGCTAGGAGCTTCAGGCTACTCTGTGTGGCTTCTGA
GTCCCATGGGGAAGTGGCGGGTATGGCCTCAGCATCAGGTCATTCAGTCCTGAGTCTATGGCAGGTAGGCTCCTAGTCGCCAGTATG
TCCCCACTTTGTCCCCCAGAGTGGCTCCAGTGAGAAGCGTGAGCTGCGTCAGTTTCAGTTCATGGCCTGGCCAGACCATGGAGTTCC
TGAGTACCCAACTCCCATCCTGGCCTTCCTACGACGGGTCAAGGCCTGCAACCCCCTAGACGCAGGGCCCATGGTGGTGCACTGCAG
GTGAGAGGGTACAGTGCCACCCAGAGGGGTGGGTGGGGTGGGAGGTGGGGGCGCCTGTGCCTCAAGCTGAGCCCGTGTCCTGCAGCG
CGGGCGTGGGCCGCACCGGCTGCTTCATCGTGATTGATGCCATGTTGGACCGGATGAAGCACGAGAAGACGGTGGACATCTATGGCC
ACGTGACCTGCATGCCATCACAGAGGAACTACATGGTGCAGACGGAGGACCAGTACGTGTTCATCCATGAGGCGCTGCTGGAGGCTG
CCACGTGCGGCCACACAGAGGTGCCTGCCCGCAACCTGTATGCCCACATCCAGAAGCTGGGCCAAGTGCCTCCAGGGGAGAGTGTGA
CCGCCATGGAGCTCGAGTTCAAGGTGGGGCTCCGGTGGGCCTGCTTGGCTCCAGGGCCTAGACTGGGTCATGCAGATGACCCCCACC
CCCACAGGAAGCCTGGCCTGACCAATCCCTGCCTCTCAATAGTTGCTGGCCAGCTCCAAGGCCCACACGTCCCGCTTCATCAGCGCC
AACCTGCCCTGCAACAAGTTCAAGAACCGGCTGGTGAACATCATGCCCTACGAATTGACCCGTGTGTGTCTGCAGCCCATCCGTGGT
GTGGAGGGCTCTGACTACATCAATGCCAGCTTCCTGGATGGTTATAGGTCAGCATGCATGTCACTGCCCCACCATGCCCTACAGGGG
CCTAGCCCTGTGCCTGGCTGGTGGGGGTGGGCAGCAGAGTAGGGCCAGCCTAGAAGACCAGAGAGGGCTGGGTAGAGCAGTGAGGAC
TTCCTGGAGGAGGGGTGATCTGAGCAGGGCCCCAAGGGGCTAGGCAGCCTAAGGGGAGACTCTAGGGGCAGCAGCACCTCCAGCATG
TCCAGTCTTATGTCCACCCCAGACAGCAGAAGGCCTACATAGCTACACAGGGGCCTCTGGCAGAGAGCACCGAGGACTTCTGGCGCA
TGCTATCGGAGCACAATTCCACCATCATCGTCATGCTGACCAAGCTTCGGGAGATGGGCAGGGTGAGCCCACCCTTTCCCCCAGGGC
CCCTGTCATACCTGGGAGAACACCAGCCACCCTTGGGGGAGCTGCCGCCTATGTTACTGTCTCCTTTGACACCCCAGCTGCTTGTCA
GCATGGCCTCAGGCGCCCGTTATTACTACCTGAGGCATCTGTCCCAGAATCCTGTGAAGCCTGGCACCCCTCCCCCATTCCTTCTCA
CCTGATTATGGGGGCCCGACCCTCTGTCCACAGGAGAAATGCCACCAGTACTGGCCAGCAGAGCGCTCTGCTCGCTACCAGTACTTT
GTTGTTGACCCGATGGCTGAGTACAACATGCCCCAGTATATCCTGCGTGAGTTCAAGGTCACGGATGCCCGGGTGAGTGAGTGCATT
GAGTGTGTCCATAACGCTGCCTGTCCACACGCTGGGTGGATGGCTGCCTGCATGGTACCTTAGCTCAAGCTTCAGAAATCTGAGGCG
GTGGGTGGGTATTAGGGTGTGAGCACATCTCCCCCTGTGGCCTCGGGTGCAGTGACACAGATGCATGCCTGTATCATGGTACTACCC
TGGTCTAGTCCAGAGGGGTGGCTGCCTAAGGCACGAATTCTAATCATGTACCCCACCCACCTTTCCCAGGATGGGCAGTCAAGGACA
ATCCGGCAGTTCCAGTTCACAGACTGGCCAGAGCAGGGCGTGCCCAAGACAGGCGAGGGATTCATTGACTTCATCGGGCAGGTGCAT
AAGACCAAGGAGCAGTTTGGACAGGATGGGCCTATCACGGTGCACTGCAGGTGGGACTGGCCCCCTGGAGGGCTGGGGTGGGTGGGC
CTGAAGGCCTGGCAGACCCACTGCATGAGGCAAGCAGGACTCCTGACCCAACTGTGTTTCTGAGCAGTGCTGGCGTGGGCCGCACCG
GGGTGTTCATCACTCTGAGCATCGTCCTGGAGCGCATGCGCTACGAGGGCGTGGTCGACATGTTTCAGACCGTGAAGACCCTGCGTA
CACAGCGTCCTGCCATGGTGCAGACAGAGGTAACGCAGACCAGGCTGCAGGGCCAGGGCCTTGGCAGCAGCGCTGCTGGGAACCCTA
GGCTTTAGCAACAGTTTGATGCCCACAGGCATGTGCATTCATTCATGCTGCCAACCTTTCACGTGGCCTGCGATAGGCATGGTGGTG
TGTGCTTATGGTCCTACCTACTTGGGAGGTTGATGTGGGACAATCACTTGAGGTCAGAAGTTCGAGGCTGCAGTGAGCTATGATTAT
ACCACGGCACTCCAGCCTGGGTGGCAGAGCAAGACCCTGTTGCTGAAAACAAAACAAAACAAAACAAAAATGCTGCAACATTGCCTG
TGCTGCCTGGGGGCTCAGGGGATTGATGAGTAAGATGTGTTCTTTCATTCGGCAGCTCTCTGCTGAGCCCCAGTGGTGTGCCTGGCT
CTGGGCAAGGTTGCACAGAGCAATCCTTATGGGGTGCCCAGTCAGGAGCAGAGAAACATGATTGGGATGGCAGATGGAAGGCAGGTA
GATGTGGGGGCCTGAGAGAATGACAGGAGAGAGATGAGCCTTCAGAGGCTCTTTCAGGCTCCCCCGCACACTGCCTGATGTAGCTGC
CAGGGTCCAGCACCTGCTCTTGGCCAGCAGAGGCTAACTCCATGGCTGCAGTGTGAGTGTCAGCTGTGTAGTGGGGGTGTCCACTGG
CGCGACCCACACTGACCAGCCCCCTATCCTGGCAGGACCAGTATCAGCTGTGCTACCGTGCGGCCCTGGAGTACCTCGGCAGCTTTG
ACCACTATGCAACGTAACTACCGCTCCCCTCTCCTCCGCCACCCCCGCCGTGGGGCTCCGGAGGGGACCCAGCTCCTCTGAGCCATA
CCGACCATCGTCCAGCCCTCCTACGCAGATGCTGTCACTGGCAGAGCACAGCCCACGGGGATCACAGCGTTTCAGGAACGTTGCCAC
ACCAATCAGAGAGCCTAGAACATCCCTGGGCAAGTGGATGGCCCAGCAGGCAGGCACTGTGGCCCTTCTGTCCACCAGACCCACCTG
GAGCCCGCTTCAAGCTCTCTGTTGCGCTCCCGCATTTCTCATGCTTCTTCTCATGGGGTGGGGTTGGGGCAAAGCCTCCTTTTTAAT
ACATTAAGTGGGGTAGACTGAGGGATTTTAGCCTCTTCCCTCTGATTTTTCCTTTCGCGAATCCGTATCTGCAGAATGGGCCACTGT
AGGGGTTGGGGTTTATTTTGTTTTGTTTTTTTTTTTCTTGAGTTCACTTTCGATCCTTATTTTGTATGACTTCTGCTGAAGGACAGA
ACATTGCCTTCCTCGTGCAGAGCTGGGGCTGCCAGCCTGAGCGGAGGCTCGGCCGTGGGCCGGGAGGCAGTGCTGATCCGGCTGCTC
CTCCAGCCCTTCAGACGAGATCCTGTTTCAGCTAAATGCAGGGAAACTCAATGTTTTTTTAAGTTTTGTTTTCCCTTTAAAGCCTTT
TTTTAGGCCACATTGACAGTGGTGGGCGGGGAGAAGATAGGGAACACTCATCCCTGGTCGTCTATCCCAGTGTGTGTTTAACATTCA
CAGCCCAGAACCACAGATGTGTCTGGGAGAGCCTGGCAAGGCATTCCTCATCACCATCGTGTTTGCAAAGGTTAAAACAAAAACAAA
AAACCACAAAAATAAAAAACAAAAAAAACAAAAAACCCAAGAAAAAAAAAAAGAGTCAGCCCTTGGCTTCTGCTTCAAACCCTCAAG
AGGGGAAGCAACTCCGTGTGCCTGGGGTTCCCGAGGGAGCTGCTGGCTGACCTGGGCCCACAGAGCCTGGCTTTGGTCCCCAGCATT
GCAGTATGGTGTGGTGTTTGTAGGCTGTGGGGTCTGGCTGTGTGGCCAAGGTGAATAGCACAGGTTAGGCTGTGTGCCACACCCCAT
GCACCTCAGGGCCAAGCGGGGGCGTGGCTGGCCTTTCAGGTCCAGGCCAGTGGGCCTGGTAGCACATGTCTGTCCTCAGAGCAGGGG
CCAGATGATTTTCCTCCCTGGTTTGCAGCTGTTTTCAAAGCCCCCGATAATCGCTCTTTTCCACTCCAAGATGCCCTCATAAACCAA
TGTGGCAAGACTACTGGACTTCTATCAATGGTACTCTAATCAGTCCTTATTATCCCAGCTTGCTGAGCGGCAGGGACAGCGCCTCTT
CCTCTGGGCAGCGCTATCTAGATAGGTAAGTGGGGGCGGGGAAGGGTGCATAGCTGTTTTAGCTGAGGGACGTGGTGCCGACGTCCC
CAAACCTAGCTAGGCTAAGTCAAGATCAACATTCCAGGGTTGGTAATGTTGGATGATGAAACATTCATTTTTACCTTGTGGATGCTA
GTGCTGTAGAGTTCACTGTTGTACACAGTCTGTTTTCTATTTGTTAAGAAAAACTACAGCATCATTGCATAATTCTTGATGGTAATA
AATTTGAATAATCAGATTTCTTAC
HUMAN mRNA SEQUENCE: hR28-021.1 (Seq ID No: 67)
GGCGCCGCCGGGGGCGCGCACGGCAGGGGCAGGGGCGCGGGCGCGAGCGCGAGGGGAGCGCGCGGCTGGAGCTGGCGCGGGAGCGGC
GGGAGCGGTGGCGGCGGCAGAGGCGGCGGCTCCAGCTTCGGCTCCGGCTCGGGCTCGGGCTCCGGCTCCGGCTCCGGCTCCGGCTCC
AGCTCGGGTGGCGGTGGCGGGAGCGGGACCAGGTGGAGGCGGCGGCGGCAGAGGAGTGGGAGCAGCGGCCCTAGCGGCTTGCGGGGG
GACATGCGGACCGACGGCCCCTGGATAGGCGGAAGGAGTGGAGGCCCTGGTGCCCGGCCCTTGGTGCTGAGTATCCAGCAAGAGTGA
CCGGGGTGAAGAAGCAAAGACTCGGTTGATTGTCCTGGGCTGTGGCTGGCTGTGGAGCTAGAGCCCTGGATGGCCCCTGAGCCAGCC
CCAGGGAGGACGATGGTGCCCCTTGTGCCTGCACTGGTGATGCTTGGTTTGGTGGCAGGCGCCCATGGTGACAGCAAACCTGTCTTC
ATTAAAGTCCCTGAGGACCAGACTGGGCTGTCAGGAGGGGTAGCCTCCTTCGTGTGCCAAGCTACAGGAGAACCCAAGCCGCGCATC
ACATGGATGAAGAAGGGGAAGAAAGTCAGCTCCCAGCGCTTCGAGGTCATTGAGTTTGATGATGGGGCAGGGTCAGTGCTTCGGATC
CAGCCATTGCGGGTGCAGCGAGATGAAGCCATCTATGAGTGTACAGCTACTAACAGCCTGGGTGAGATCAACACTAGTGCCAAGCTC
TCAGTGCTCGAAGAGGAACAGCTGCCCCCTGGGTTCCCTTCCATCGACATGGGGCCTCAGCTGAAGGTGGTGGAGAAGGCACGCACA
GCCACCATGCTATGTGCCGCAGGCGGAAATCCAGACCCTGAGATTTCTTGGTTCAAGGACTTCCTTCCTGTAGACCCTGCCACGAGC
AACGGCCGCATCAAGCAGCTCCGTTCAGGTGCCTTGCAGATAGAGAGCAGTGAGGAATCCGACCAAGGCAAGTACGAGTGTGTGGCG
ACCAACTCGGCAGGCACACGTTACTCAGCCCCTGCGAACCTGTATGTGCGAGTGCGCCGCGTGGCTCCTCGTTTCTCCATCCCTCCC
AGCAGCCAGGAGGTGATGCCAGGCGGCAGCGTGAACCTGACATGCGTGGCAGTGGGTGCACCCATGCCCTACGTGAAGTGGATGATG
GGGGCCGAGGAGCTCACCAAGGAGGATGAGATGCCAGTTGGCCGCAACGTCCTGGAGCTCAGCAATGTCGTACGCTCTGCCAACTAC
ACCTGTGTGCCCATCTCCTCGCTGGGCATGATCGAGGCCACAGCCCAGGTCACAGTGAAAGCTCTTCCAAAGCCTCCGATTGATCTT
GTGGTGACAGAGACAACTGCCACCAGTGTCACCCTCACCTGGGACTCTGGGAACTCGGAGCCTGTAACCTACTATGGCATCCAGTAC
CGCGCAGCGGGCACGGAGGGCCCCTTTCAGGAGGTGGATGGTGTGGCCACCACCCGCTACAGCATTGGCGGCCTCAGCCCTTTCTCG
GAATATGCCTTCCGCGTGCTGGCGGTGAACAGCATCGGGCGAGGGCCGCCCAGCGAGGCAGTGCGGGCACGCACGGGAGAACAGGCG
CCCTCCAGCCCACCGCGCCGCGTGCAGGCACGCATGCTGAGCGCCAGCACCATGCTGGTGCAGTGGGAGCCTCCCGAGGAGCCCAAC
GGCCTGGTGCGGGGATACCGCGTCTACTATACTCCGGACTCCCGCCGCCCCCCGAACGCCTGGCACAAGCACAACACCGACGCGGGG
CTCCTCACGACCGTGGGCAGCCTGCTGCCTGGCATCACCTACAGCCTGCGCGTGCTTGCCTTCACCGCCGTGGGCGATGCCCCTCCC
AGCCCCACCATCCAGGTCAAGACGCAGCAGGGAGTGCCTGCCCAGCCCGCGGACTTCCAGGCCGAGGTGGAGTCGGACACCAGGATC
CAGCTCTCGTGGCTGCTGCCCCCTCAGGAGCGGATCATCATGTATGAACTGGTGTACTGGGCGGCAGAGGACGAAGACCAACAGCAC
AAGGTGACCTTCGACCCAACCTCCTCCTACACACTAGAGGACCTGAAGCCTGACACACTCTACCGCTTCCAGCTGGCTGCACGCTCG
GATATGGGGGTGGGCGTCTTCACCCCCACCATTGAGGCCCGCACAGCCCAGTCCACCCCCTCCGCCCCTCCCCAGAAGGTGATGTGT
GTGAGCATGGGCTCCACCACGGTCCGGGTAAGTTGGGTCCCGCCGCCTGCCGACAGCCGCAACGGCGTTATCACCCAGTACTCCGTG
GCCTACGAGGCGGTGGACGGCGAGGACCGCGGGCGGCATGTGGTGGATGGCATCAGCCGTGAGCACTCCAGCTGGGACCTGGTGGGC
CTGGAGAAGTGGACGGAGTACCGGGTGTGGGTGCGGGCACACACAGACGTGGGCCCCGGCCCCGAGAGCAGCCCGGTGCTGGTGCGC
ACCGATGAGGACGTGCCCAGCGGGCCTCCGCGGAAGGTGGAGGTGGAGCCACTGAACTCCACTGCTGTGCATGTCTACTGGAAGCTG
CCTGTCCCCAGCAAGCAGCATGGCCAGATCCGCGGCTACCAGGTCACCTACGTGCGGCTGGAGAATGGCGAGCCCCGTGGACTCCCC
ATCATCCAAGACGTCATGCTAGCCGAGGCCCAGTGGCGGCCAGAGGAGTCCGAGGACTATGAAACCACTATCAGCGGCCTGACCCCG
GAGACCACCTACTCCGTTACTGTTGCTGCCTATACCACCAAGGGGGATGGTGCCCGCAGCAAGCCCAAAATTGTCACTACAACAGGT
GCAGTCCCAGGCCGGCCCACCATGATGATCAGCACCACGGCCATGAACACTGCGCTGCTCCAGTGGCACCCACCCAAGGAACTGCCT
GGCGAGCTGCTGGGCTACCGGCTGCAGTACTGCCGGGCCGACGAGGCGCGGCCCAACACCATAGATTTCGGCAAGGATGACCAGCAC
TTCACAGTCACCGGCCTGCACAAGGGGACCACCTACATCTTCCGGCTTGCTGCCAAGAACCGGGCTGGCTTGGGTGAGGAGTTCGAG
AAGGAGATCAGGACCCCCGAGGACCTGCCCAGCGGCTTCCCCCAAAACCTGCATGTGACAGGACTGACCACGTCTACCACAGAACTG
GCCTGGGACCCGCCAGTGCTGGCGGAGAGGAACGGGCGCATCATCAGCTACACCGTGGTGTTCCGAGACATCAACAGCCAACAGGAG
CTGCAGAACATCACGACAGACACCCGCTTTACCCTTACTGGCCTCAAGCCAGACACCACTTACGACATCAAGGTCCGCGCATGGACC
AGCAAAGGCTCTGGCCCACTCAGCCCCAGCATCCAGTCCCGGACCATGCCGGTGGAGCAAGTGTTTGCCAAGAACTTCCGGGTGGCG
GCTGCAATGAAGACGTCTGTGCTGCTCAGCTGGGAGGTTCCCGACTCCTATAAGTCAGCTGTGCCCTTTAAGATTCTGTACAATGGG
CAGAGTGTGGAGGTGGACGGGCACTCGATGCGGAAGCTGATCGCAGACCTGCAGCCCAACACAGAGTACTCGTTTGTGCTGATGAAC
CGTGGCAGCAGCGCAGGGGGCCTGCAGCACCTGGTGTCCATCCGCACAGCCCCCGACCTCCTGCCTCACAAGCCGCTGCCTGCCTCT
GCCTACATAGAGGACGGCCGCTTCGATCTCTCCATGCCCCATGTGCAAGACCCCTCGCTTGTCAGGTGGTTCTACATTGTTGTGGTA
CCCATTGACCGTGTGGGCGGGAGCATGCTGACGCCAAGGTGGAGCACACCCGAGGAACTGGAGCTGGACGAGCTTCTAGAAGCCATC
GAGCAAGGCGGAGAGGAGCAGCGGCGGCGGCGGCGGCAGGCAGAACGTCTGAAGCCATATGTGGCTGCTCAACTGGATGTGCTCCCG
GAGACCTTTACCTTGGGGGACAAGAAGAACTACCGGGGCTTCTACAACCGGCCCCTGTCTCCGGACTTGAGCTACCAGTGCTTTGTG
CTTGCCTCCTTGAAGGAACCCATGGACCAGAAGCGCTATGCCTCCAGCCCCTACTCGGATGAGATCGTGGTCCAGGTGACACCAGCC
CAGCAGCAGGAGGAGCCGGAGATGCTGTGGGTGACGGGTCCCGTGCTGGCAGTCATCCTCATCATCCTCATTGTCATCGCCATCCTC
TTGTTCAAAAGGAAAAGGACCCACTCTCCGTCCTCTAAGGATGAGCAGTCGATCGGACTGAAGGACTCCTTGCTGGCCCACTCCTCT
GACCCTGTGGAGATGCGGAGGCTCAACTACCAGACCCCAGGTATGCGAGACCACCCACCCATCCCCATCACCGACCTGGCGGACAAC
ATCGAGCGCCTCAAAGCCAACGATGGCCTCAAGTTCTCCCAGGAGTATGAGTCCATCGACCCTGGACAGCAGTTCACGTGGGAGAAT
TCAAACCTGGAGGTGAACAAGCCCAAGAACCGCTATGCGAATGTCATCGCCTACGACCACTCTCGAGTCATCCTTACCTCTATCGAT
GGCGTCCCCGGGAGTGACTACATCAATGCCAACTACATCGATGGCTACCGCAAGCAGAATGCCTACATCGCCACGCAGGGCCCCCTG
CCCGAGACCATGGGCGATTTCTGGAGAATGGTGTGGGAACAGCGCACGGCCACTGTGGTCATGATGACACGGCTGGAGGAGAAGTCC
CGGGTAAAATGTGATCAGTACTGGCCAGCCCGTGGCACCGAGACCTGTGGCCTTATTCAGGTGACCCTGTTGGACACAGTGGAGCTG
GCCACATACACTGTGCGCACCTTCGCACTCCACAAGAGTGGCTCCAGTGAGAAGCGTGAGCTGCGTCAGTTTCAGTTCATGGCCTGG
CCAGACCATGGAGTTCCTGAGTACCCAACTCCCATCCTGGCCTTCCTACGACGGGTCAAGGCCTGCAACCCCCTAGACGCAGGGCCC
ATGGTGGTGCACTGCAGCGCGGGCGTGGGCCGCACCGGCTGCTTCATCGTGATTGATGCCATCTTGGAGCGGATGAAGCACGAGAAG
ACGGTGGACATCTATGGCCACGTGACCTGCATGCGATCACAGAGGAACTACATGGTGCAGACGGAGGACCAGTACGTGTTCATCCAT
GAGGCGCTGCTGGAGGCTGCCACGTGCGGCCACACAGAGGTGCCTGCCCGCAACCTGTATGCCCACATCCAGAAGCTGGGCCAAGTG
CCTCCAGGGGAGAGTGTGACCGCCATGGAGCTCGAGTTCAAGTTGCTGGCCAGCTCCAAGGCCCACACGTCCCGCTTCATCAGCGCC
ACCTGCCCTGCAACAAGTTCAAGAACCGGCTGGTGAACATCATGCCCTACGAATTGACCCGTGTGTGTCTGCAGCCCATCCGTAGGT
GTGGAGGGCTCTGACTACATCAATGCCAGCTTCCTGGATGGTTATAGACAGCAGAAGGCCTACATAGCTACACAGGGGCCTCTGGCA
GAGAGCACCGAGGACTTCTGGCGCATGCTATGGGAGCACAATTCCACCATCATCGTCATGCTGACCAAGCTTCGGGAGATGGGCAGG
GAGAAATGCCACCAGTACTGGCCAGCAGAGCGCTCTGCTCGCTACCAGTACTTTGTTGTTGACCCGATGGCTGAGTACAACATGCCC
CAGTATATCCTGCGTGAGTTCAAGGTCACGGATGCCCGGGATGGGCAGTCAAGGACAATCCGGCAGTTCCAGTTCACAGACTGGCCA
GAGCAGGGCGTGCCCAAGACAGGCGAGGGATTCATTGACTTCATCGGGCAGGTGCATAAGACCAAGGAGCAGTTTGGACAGGATGGG
CCTATCACGGTGCACTGCAGTGCTGGCGTGGGCCGCACCGGGGTGTTCATCACTCTGAGCATCGTCCTGGAGCGCATGCGCTACGAG
GGCGTGGTCGACATGTTTCAGACCGTGAAGACCCTGCGTACACAGCGTCCTGCCATGGTGCAGACAGAGGACCAGTATCAGCTGTGC
TACCGTGCGGCCCTGGAGTACCTCGGCAGCTTTGACCACTATGCAACGTAACTACCGCTCCCCTCTCCTCCGCCACCCCCGCCGTGG
GGCTCCGGAGGGGACCCAGCTCCTCTGAGCCATACCGACCATCGTCCAGCCCTCCTACGCAGATGCTGTCACTGGCAGAGCACAGCC
CACGGGGATCACAGCGTTTCAGGAACGTTGCCACACCAATCAGAGAGCCTAGAACATCCCTGGGCAAGTGGATGGCCCAGCAGGCAG
GCACTGTGGCCCTTCTGTCCACCAGACCCACCTGGAGCCCGCTTCAACCTCTCTGTTGCGCTCCCGCATTTCTCATGCTTCTTCTCA
TGGGGTGGGGTTGGGGCAAAGCCTCCTTTTTAATACATTAAGTGGGGTAGACTGAGGGATTTTAGCCTCTTCCCTCTGATTTTTCCT
TTCGCGAATCCGTATCTGCAGAATGGGCCACTGTAGGGGTTGGGGTTTATTTTGTTTTGTTTTTTTTTTTCTTGATTTGTATGACTT
CTGCTGAAGGACAGAACATTGCCTTCCTCGTGCAGAGCTGGGGCTGCCAGCCTGAGCGGAGGCTCGGCCGTGGGCCGGGAGGCAGTG
CTGATCCGGCTGCTCCTCCAGCCCTTCAGACGAGATCCTGTTTCAGCTAAATGCAGGGAAACTCAATGTTTTTTTAAGTTTTGTTTT
CCCTTTAAAGCCTTTTTTTAGGCCACATTGACAGTGGTGGGCGGGGAGAAGATAGGGAACACTCATCCCTGGTCGTCTATCCCAGTG
TGTGTTTAACATTCACAGCCCAGAACCACAGATGTGTCTGGGAGAGCCTGGCAAGGCATTCCTCATCACCATCGTGTTTGCAAAGGT
TAAAACAAAAACAAAAAACCACAAAAATAAAAAACAAAAAAAACAAAAAACCCAAGAAAAAAAAAAAGAGTCAGCCCTTGGCTTCTG
CTTCAAACCCTCAAGAGGGGAAGCAACTCCGTGTGCCTGGGGTTCCCGAGGGAGCTGCTGGCTGACCTGGGCCCACAGAGCCTGGCT
TTGGTCCCCAGCATTGCACTATGGTGTGGTGTTTGTAGGCTGTGGGGTCTGGCTGTGTGGCCAAGGTGAATAGCACAGGTTAGGGTG
TGTGCCACACCCCATGCACCTCAGGGCCAAGCGGGGGCGTGGCTGGCCTTTCAGGTCCAGGCCAGTGGGCCTGGTAGCACATGTCTG
TCCTCAGAGCAGGGGCCAGATGATTTTCCTCCCTGGTTTGCAGCTGTTTTCAAAGCCCCCGATAATCGCTCTTTTCCACTCCAAGAT
GCCCTCATAAACCAATGTGGCAAGACTACTGGACTTCTATCAATGGTACTCTAATCAGTCCTTATTATCCCAGCTTGCTGAGGGGCA
GGGAGAGCGCCTCTTCCTCTGGGCAGCGCTATCTAGATAGGTAAGTGGGGGCGGGGAAGGGTGCATAGCTGTTTTAGCTGAGGGACG
TGGTGCCGACGTCCCCAAACCTAGCTAGGCTAAGTCAAGATCAACATTCCAGGGTTGGTAATGTTGGATGATGGGGCATTCATTTTT
ACCTTGTGGATGCTAGTGCTGTAGAGTTCACTGTTGTACACAGTCTGTTTTCTATTTGTTAAGAAAAACTACAGCATCATTGCATAA
TTCTTGATGGTAATAAATTTGAATAATCAGATTTCTTAC
HUMAN PROTEIN SEQUENCE: hP28-021.1 (Seq ID No: 68)
MAPEPAPGRTMVPLVPALVMLGLVAGAHGDSKPVFIKVPEDQTGLSGGVASFVCQATGEPKPRITWMKKGKKVSSQRFEVIEFDDGA
GSVLRIQPLRVQRDEAIYECTATNSLGEINTSAKLSVLEEEQLPPGFPSIDMGPQLKVVEKARTATMLCAAGCNPDPEISWFKDFLP
VDPATSNGRIKQLRSGALQIESSEESDQGKYECVATNSAGTRYSAPANLYVRVRRVAPRFSIPPSSQEVMPGGSVNLTCVAVGAPMP
YVKWMNGAEELTKEDEMPVGRNVLELSNVVRSAMYTCVAISSLGMIEATAQTVKALPKPPIDLVVTETTATSVTLTWDSGANSEPVT
YYGIQYRAAGTEGPFQEVDGVATTRYSIGGLSPFSEYAFRVLAVNSIGRGPPSEAVRARTGEQAPSSPPRRVQARMLSASTMLVQWE
PPEEPNGLVRGYRVYYTPDSRRPPNAWHKHNTDAGLLTTVGSLLPGITYSLRVLAFTAVGDGPPSPTIQVKTQQGVPAQPADFQAEV
ESDTRIQLSWLLPPQERIIMYELVYWAAEDEDQQHKVTFDPTSSYTLEDLKPDTLYRFQLAARSDMGVGVFTPTIEARTAQSTPSAP
PQKVMCVSMGSTTVRVSWVPPPADSRNGVITQYSVAYEAVDGEDRGRHVVDGISREHSSWDLVGLEKWTEYRVWVRAHTDVGPGPES
SPVLVRTDEDVPSGPPRKVEVEPLNSTAVHVYWKLPVPSKQHGQIRGYQVTYVRLENGEPRGLPIIQDVMLAEAQWRPEESEDYETT
ISGLTPETTYSVTVAAYTTKGDGARSKPKIVTTTGAVPGRPTMMISTTAMNTALLQWHPPKELPGELLGYRLQYCRADEARPNTIDF
GKDDQHFTVTGLHKGTTYIFRLAAKNRAGLGEEFEKEIRTPEDLPSGFPQNLHVTGLTTSTTELAWDPPVLAERNGRIISYTVVFRD
INSQQELQNITTDTRFTLTGLKPDTTYDIKVRAWTSKGSGPLSPSIQSRTMPVEQVFAKNFRVAAAMKTSVLLSWEVPDSYKSAVPF
KILYNGQSVEVDGHSMRKLIADLQPNTEYSFVLMNRGSSAGGLQHLVSIRTAPDLLPHKPLPASAYIEDGRFDLSMPHVQDPSLVRW
FYIVVVPIDRVGGSMLTPRWSTPEELELDELLEAIEQGGEEQRRRRRQAERLKPYVAAQLDVLPETFTLGDKKNYRGFYNRPLSPDL
SYQCFVLASLKEPMDQKRYASSPYSDEIVVQVTPAQQQEEPEMLNVTGPVLAVILIILIVIAILLFKRKRTHSPSSKDEQSIGLKDS
LLAHSSDPVEMRRLNYQTPGMRDHPPIPITDLANNIERLKANDGLKFSQEYESIDPGQQFTWENSNLEVNKPKNRYANVIAYDHSRV
ILTSIDGVPGSDYINANYIDGYRKQNAYIATQGPLPETMGDFWRMVWEQRTATVVMMTRLEEKSRVKCDQYWPARGTETCGLIQVTL
LDTVELATYTVRTFALHKSGSSEKRELRQFQFMAWPDHGVPEYPTPILAFLRRVKACNPLDAGPMVVHCSAGVGRTGCFIVIDAILE
RMKHEKTVDIYGHVTCMRSQRNYMVQTEDQYVFIHEALLEAATCGHTEVPARNLYAHIQKLGQVPPGESVTAMELEFKLLASSKAHT
SRFISANLPCNKFKNRLVNIMPYELTRVCLQPIRGVEGSDYINASFLDGYRQQKAYIATQGPLAESTEDFWRMLWEHNSTIIVMLTK
LREMGREKCNQYWPAERSARYQYFVVDPMAEYNMPQYILREFKVTDARDGQSRTIRQFQFTDWPEQGVPKTGEGFIDFIGQVNKTKE
QFGQDGPITVHCSAGVGRTGVFITLSIVLERMRYEGVVDMFQTVKTLRTQRPAMVQTEDQYQLCYRAALEYLGSFDHYAT*
HUMAN mRNA SEQUENCE: hR28-021.2 (Seq ID No: 69)
GGCGCCGCCGGGGGCGCGCACGGCAGGGGCAGGGGCGCGGGCGCGAGCGCGAGGGGAGCGCGCGGCTGGAGCTGGCGCGGGAGCGGC
GGGAGCGGTGGCGGCGGCAGAGGCGGCGGCTCCAGCTTCGGCTCCGGCTCGGGCTCGGGCTCCOGCTCCGGCTCCGGCTCCGGCTCC
AGCTCGGGTGGCGGTGGCGGGAGCGGGACCAGGTGGAGGCGGCGGCGGCAGAGGAGTGGGAGCAGCGGCCCTAGCGGCTTGCGGGGG
GACATGCGGACCGACGGCCCCTGGATAGGCGGAAGGAGTGGAGGCCCTGGTGCCCGGCCCTTGGTGCTGAGTATCCAGCAAGAGTGA
CCGGGGTGAAGAAGCAAAGACTCGGTTGATTGTCCTCGGCTGTGGCTGGCTGTGGAGCTAGAGCCCTGGATGGCCCCTGAGCCAGCC
CCAGGGAGGACGATGGTGCCCCTTGTGCCTGCACTGGTGATGCTTGGTTTGGTGGCAGGCGCCCATGGTGACAGCAAACCTGTCTTC
ATTAAAGTCCCTGAGGACCAGACTGGGCTGTCAGGAGGGGTAGCCTCCTTCGTGTGCCAAGCTACAGGAGAACCCAAGCCGCGCATC
ACATGGATGAAGAAGGGGAAGAAAGTCAGCTCCCAGCGCTTCGAGGTCATTGAGTTTGATGATGGGGCAGGGTCAGTGCTTCGCATC
CAGCCATTGCGGGTGCAGCGAGATGAGCCATCTATGAGTGTACAGCTACTAACAGCCTGGGTGAGAATCAACACTAGTGCCAAGCTC
TCAGTGCTCGAAGAGGAACAGCTGCCCCCTGGGTTCCCTTCCATCGACATGGGGCCTCAGCTGAAGGTGGTGGAGAAGGCACGCACA
GCCACCATGCTATGTGCCGCAGGCGGAAATCCAGACCCTGAGATTTCTTGGTTCAAOGACTTCCTTCCTGTAGACCCTGCCACGAGC
AACGGCCGCATCAAGCAGCTGCGTTCAGGTGCCTTGCAGATAGAGAGCAGTGAGGAATCCCACCAAGGCAAGTACGAGTGTGTGGCG
ACCAACTCGGCAGGCACACGTTACTCAGCCCCTGCGAACCTGTATGTGCGAGTGCGCCGCGTGGCTCCTCGTTTCTCCATCCCTCCC
AGCAGCCAGGAGGTGATGCCAGGCGGCAGCGTGAACCTGACATGCGTGGCAGTGGGTGCACCCATGCCCTACGTGAAGTGGATGATG
GGGGCCGAGGAGCTCACCAAGGAGGATGAGATGCCAGTTGGCCGCAACGTCCTGGAGCTCAGCAATGTCGTACGCTCTGCCAACTAC
ACCTGTGTGGCCATCTCCTCGCTGGGCATGATCGAGGCCACAGCCCAGGTCACAGTGAAAGCTCTTCCAAAGCCTCCGATTGATCTT
GTGGTGACAGAGACAACTGCCACCAGTGTCACCCTCACCTGGGACTCTOGGAACTCGGAGCCTGTAACCTACTATGGCATCCAGTAC
CGCGCAGCGGGCACGGAGGGCCCCTTTCAGGAGGTGGATGGTGTGGCCACCACCCGCTACAGCATTGGCGGCCTCAGCCCTTTCTCG
GAATATGCCTTCCGCGTGCTGGCGGTGAACAGCATCGGGCGAGGGCCGCCCAGCGAGGCAGTGCGGGCACGCACGGGAGAACAGGCG
CCCTCCAGCCCACCGCGCCGCGTGCAGGCACGCATGCTGAGCGCCAGCACCATGCTGGTGCAGTGGGAGCCTCCCGAGGAGCCCAAC
GGCCTGGTGCGGGGATACCGCGTCTACTATACTCCGGACTCCCGCCGCCCCCCGAACGCCTGGCACAAGCACAACACCGACGCGGGG
CTCCTCACGACCGTGGGCAGCCTGCTGCCTGGCATCACCTACAGCCTGCGCGTGCTTGCCTTCACCGCCGTGGGCGATGGCCCTCCC
AGCCCCACCATCCAGGTCAAGACGCAGCAGGGAGTGCCTGCCCAGCCCGCGGACTTCCAGGCCGAGGTGGAGTCGGACACCAGGATC
CAGCTCTCGTGGCTGCTGCCCCCTCAGGAGCGGATCATCATGTATGAACTOGTGTACTGGGCGGCAGAGGACGAAGACCAACAGCAC
AAGGTGACCTTCGACCCAACCTCCTCCTACACACTAGAGGACCTGAAGCCTGACACACTCTACCGCTTCCAGCTGGCTGCACGCTCG
GATATGGGGCTGGGCGTCTTCACCCCCACCATTGAGGCCCGCACAGCCCAGTCCACCCCCTCCGCCCCTCCCCAGAAGGTGATGTGT
GTGAGCATGGGCTCCACCACGGTCCGGGTAAGTTGGGTCCCGCCGCCTGCCGACAGCCGCAACGGCGTTATCACCCAGTACTCCGTG
GCCTACGAGGCGGTGGACGGCGAGGACCGCGGGCGGCATGTGGTGGATGGCATCAGCCGTGAGCACTCCAGCTGGGACCTGGTGGGC
CTGGAGAAGTGGACGGAGTACCGGGTGTGGGTGCGGGCACACACAGACGTGGGCCCCGGCCCCGAGAGCAGCCCGGTGCTGGTGCGC
ACCGATGAGGACGTGCCCAGCGGGCCTCCGCGGAAGGTGGAGGTGGAGCCACTGAACTCCACTGCTGTGCATGTCTACTGGAAGCTG
CCTGTCCCCAGCAAGCAGCATGGCCAGATCCGCGGCTACCAGGTCACCTACGTGCGGCTGGAGAATGGCGAGCCCCGTGGACTCCCC
ATCATCCAAGACGTCATGCTAGCCGAGGCCCAGGAAACCACTATCAGCGGCCTGACCCCGGAGACCACCTACTCCCTTACTGTTGCT
GCCTATACCACCAAGGGGGATGGTGCCCGCAGCAAGCCCAAAATTGTCACTACAACAGGTGCAGTCCCAGGCCGGCCCACCATGATG
ATCAGCACCACGGCCATGAACACTGCGCTGCTCCAGTGGCACCCACCCAAGGAACTGCCTGGCGAGCTGCTGGGCTACCGGCTGCAG
TACTGCCGGGCCGACGAGGCGCGGCCCAACACCATAGATTTCGGCAAGGATGACCAGCACTTCACAGTCACCCGCCTGCACAAGGGG
ACCACCTACATCTTCCGGCTTGCTGCCAAGAACCGGGCTGGCTTGGGTGAGGAGTTCGAGAAGGAGATCAGGACCCCCGAGGACCTG
CCCAGCGGCTTCCCCCAAAACCTGCATGTGACAGGACTGACCACGTCTACCACAGAACTGGCCTGGGACCCGCCAGTGCTGGCGGAG
AGGAACGGGCGCATCATCAGCTACACCGTGGTGTTCCGAGACATCAACAGCCAACAGGAGCTGCAGAACATCACGACAGACACCCGC
TTTACCCTTACTGGCCTCAAGCCAGACACCACTTACGACATCAAGGTCCGCGCATGGACCAGCAAAGGCTCTGGCCCACTCAGCCCC
AGCATCCAGTCCCGGACCATGCCGGTGGAGCAAGTGTTTGCCAAGAACTTCCGGGTGGCGGCTGCAATGAAGACGTCTGTGCTGCTC
AGCTGGGAGGTTCCCGACTCCTATAAGTCAGCTGTGCCCTTTAAGATTCTGTACAATGGGCAGAGTGTGGAGGTGGACGGGCACTCG
ATGCGGAAGCTGATCGCAGACCTGCAGCCCAACACAGAGTACTCGTTTGTGCTGATGAACCGTGGCAGCAGCGCAGGGGGCCTGCAG
CACCTGGTGTCCATCCGCACAGCCCCCGACCTCCTGCCTCACAAGCCGCTGCCTGCCTCTGCCTACATAGAGGACGGCCGCTTCGAT
CTCTCCATGCCCCATGTGCAAGACCCCTCGCTTGTCAGGTGGTTCTACATTGTTGTGGTACCCATTGACCGTGTGGGCGGGAGCATG
CTGACGCCAAGGTGGAGCACACCCGAGGAACTGGAGCTGGACGAGCTTCTAGAAGCCATCGAGCAAGGCGGAGAGGAGCAGCGGCGG
CGGCGGCGGCAGGCAGAACGTCTGAAGCCATATGTGGCTGCTCAACTGGATGTGCTCCCGGAGACCTTTACCTTGGGGGACAAGAAG
AACTACCGGGGCTTCTACAACCGGCCCCTGTCTCCGGACTTGAGCTACCAGTGCTTTGTGCTTGCCTCCTTGAAGGAACCCATGGAC
CAGAAGCGCTATGCCTCCAGCCCCTACTCGGATGAGATCGTGGTCCAGGTGACACCAGCCCAGCAGCAGGAGGAGCCGGAGATGCTG
TGGGTGACGGGTCCCGTGCTGGCAGTCATCCTCATCATCCTCATTGTCATCGCCATCCTCTTGTTCAAAAGGAAAAGGACCCACTCT
CCGTCCTCTAAGGATGAGCAGTCGATCGGACTGAAGGACTCCTTGCTGGCCCACTCCTCTGACCCTGTGGAGATGCGGAGGCTCAAC
TACCAGACCCCAGGTATGCGAGACCACCCACCCATCCCCATCACCGACCTGGCGGACAACATCGAGCGCCTCAAAGCCAACGATGGC
CTCAAGTTCTCCCAGGAGTATGAGTCCATCGACCCTGGACAGCAGTTCACGTGGGAGAATTCAAACCTGGAGGTGAACAAGCCCAAG
AACCGCTATGCGAATGTCATCGCCTACGACCACTCTCGAGTCATCCTTACCTCTATCGATGGCGTCCCCGGGAGTGACTACATCAAT
GCCAACTACATCGATGGCTACCGCAAGCAGAATGCCTACATCGCCACGCAGGGCCCCCTGCCCGAGACCATGGGCGATTTCTGGAGA
ATGGTGTGGGAACAGCGCACGGCCACTGTGGTCATGATGACACGGCTGGAGGAGAAGTCCCGGGTAAAATGTGATCAGTACTGGCCA
GCCCGTGGCACCGAGACCTGTGGCCTTATTCAGGTGACCCTGTTGGACACAGTGGAGCTGGCCACATACACTGTGCGCACCTTCGCA
CTCCACAAGAGTGGCTCCAGTGAGAAGCGTGAGCTGCGTCAGTTTCAGTTCATGGCCTGGCCAGACCATGGAGTTCCTGAGTACCCA
ACTCCCATCCTGGCCTTCCTACGACGGGTCAAGGCCTGCAACCCCCTAGACGCAGGGCCCATGGTGGTGCACTGCAGCGCGGGCGTG
GGCCGCACCGGCTGCTTCATCGTGATTGATGCCATGTTGGAGCGGATGAAGCACGAGAAGACGGTGGACATCTATGGCCACGTGACC
TGCATGCGATCACAGAGGAACTACATGGTGCAGACGGAGGACCAGTACGTGTTCATCCATGAGGCGCTGCTGGAGGCTGCCACGTGC
GGCCACACAGAGGTGCCTGCCCGCAACCTGTATGCCCACATCCAGAAGCTGGGCCAAGTGCCTCCAGGGGAGAGTGTGACCGCCATG
GAGCTCGAGTTCAAGTTGCTGGCCAGCTCCAAGGCCCACACGTCCCGCTTCATCAGCGCCAACCTGCCCTGCAACAAGTTCAAGAAC
CGGCTGGTGAACATCATGCCCTACGAATTGACCCGTGTGTGTCTGCAGCCCATCCGTGGTGTGGAGGGCTCTGACTACATCAATGCC
AGCTTCCTGGATGGTTATAGACAGCAGAAGGCCTACATAGCTACACAGGGGCCTCTGGCAGAGAGCACCGAGGACTTCTGGCGCATG
CTATGGGAGCACAATTCCACCATCATCGTCATGCTGACCAAGCTTCGGGAGATGGGCAGGGAGAAATGCCACCAGTACTGGCCAGCA
GAGCGCTCTGCTCGCTACCAGTACTTTGTTGTTGACCCGATGGCTGAGTACAACATGCCCCAGTATATCCTGCGTGAGTTCAAGGTC
ACGGATGCCCGGGATGGGCAGTCAAGGACAATCCGGCAGTTCCAGTTCACAGACTGGCCAGAGCAGGGCGTGCCCAAGACAGGCGAG
GGATTCATTGACTTCATCGGGCAGGTGCATAAGACCAAGGAGCAGTTTGGACAGGATGGGCCTATCACGGTGCACTGCAGTGCTGGC
GTGGGCCGCACCGGGGTGTTCATCACTCTGAGCATCGTCCTGGAGCGCATGCGCTACGAGGGCGTGGTCGACATGTTTCAGACCGTG
AAGACCCTGCGTACACAGCGTCCTGCCATGGTGCAGACAGAGGACCAGTATCAGCTGTGCTACCGTGCGGCCCTGGAGTACCTCGGC
AGCTTTGACCACTATGCAACGTAACTACCGCTCCCCTCTCCTCCGCCACCCCCGCCGTGGGGCTCCGGAGGGGACCCAGCTCCTCTG
AGCCATACCGACCATCGTCCAGCCCTCCTACGCAGATGCTGTCACTGGCAGAGCACAGCCCACGGGGATCACAGCGTTTCAGGAACG
TTGCCACACCAATCAGAGAGCCTAGAACATCCCTGGGCAAGTGGATGGCCCAGCAGGCAGGCACTGTGGCCCTTCTGTCCACCAGAC
CCACCTGGAGCCCGCTTCAAGCTCTCTGTTGCGCTCCCGCATTTCTCATGCTTCTTCTCATGGGGTGGGGTTGGGGCAAAGCCTCCT
TTTTAATACATTAAGTGGGGTAGACTGAGGGATTTTAGCCTCTTCCCTCTGATTTTTCCTTTCGCGAATCCGTATCTGCAGAATGGG
CCACTGTAGGGGTTGGGGTTTATTTTGTTTTGTTTTTTTTTTTCTTGATTTGTATGACTTCTGCTGAAGGACAGAACATTGCCTTCC
TCGTGCAGAOCTGGGGCTGCCAGCCTGAGCGGAGGCTCGGCCGTGGGCCGGGAGGCAGTGCTGATCCGGCTGCTCCTCCAGCCCTTC
AGACGAGATCCTGTTTCAGCTAAATGCAGGGAAACTCAATGTTTTTTTAAGTTTTGTTTTCCCTTTAAAGCCTTTTTTTAGGCCACA
TTGACAGTGGTGGGCGGGGAGAAGATAGGGAACACTCATCCCTGGTCGTCTATCCCAGTGTCTGTTTAACATTCACAGCCCAGAACC
ACAGATGTGTCTGGGAGAGCCTGGCAAGGCATTCCTCATCACCATCGTGTTTGCAAAGGTTAAAACAAAAACAAAAAACCACAAAAA
TAAAAAACAAAAAAAACAAAAAACCCAAGAAAAAAAAAAAGAGTCAGCCCTTGGCTTCTGCTTCAAACCCTCAAGAGGGOAAGCAAC
TCCGTGTGCCTGGGGTTCCCGAGGGAGCTGCTGGCTGACCTGGGCCCACAGAGCCTGGCTTTGGTCCCCAGCATTGCAGTATGGTGT
GGTGTTTGTAGGCTGTGGGGTCTGGCTGTGTGGCCAAGGTGAATAGCACAGGTTAGGGTGTGTGCCACACCCCATGCACCTCAGGGC
CAAGCGGGGGCGTGGCTGGCCTTTCAGGTCCAGGCCAGTGOGCCTGGTAGCACATGTCTGTCCTCAGAGCAGGGGCCAOATGATTTT
CCTCCCTGGTTTGCAGCTGTTTTCAAAGCCCCCGATAATCGCTCTTTTCCACTCCAAGATGCCCTCATAAACCAATGTGGCAAGACT
ACTGGACTTCTATCAATGGTACTCTAATCAGTCCTTATTATCCCAGCTTGCTGAGGCGCAGGGAGAGCGCCTCTTCCTCTGGGCAGC
GCTATCTAGATAGGTAAGTGGGGGCGGGGAAGGGTGCATAGCTGTTTTAGCTGAGGGACGTGGTGCCGACGTCCCCAAACCTAGCTA
GGCTAAGTCAAGATCAACATTCCAGGGTTGGTAATGTTGGATGATGAAACATTCATTTTTACCTTGTGGATGCTAGTGCTGTAGAGT
TCACTGTTGTACACAGTCTGTTTTCTATTTGTTAAGAAAAACTACAGCATCATTGCATAATTCTTGATGGTAATAAATTTGAATAAT
CAGATTTCTTAC
HUMAN PROTEIN SEQUENCE: hP28-021.2 (Seq ID No: 70)
MAPEPAPGRTMVPLVPALVMLGLVAGAHGDSKPVFIKVPEDQTGLSGGVASFVCQATCEPKPRITWMKKGKKVSSQRFEVIEFDDGA
GSVLRIQPLRVQRDEAIYECTATNSLGEINTSAKLSVLEEEQLPPGFPSIDMGPQLKVVEKARTATMLCAAGGNPDPEISWFKDFLP
VDPATSNGRIKQLRSGALQIESSEESDQGKYECVATNSAGTRYSAPANLYVRVRRVAPRFSIPPSSQEVMPGGSVNLTCVAVGAPMP
YVKWMMGAEELTKEDEMPVGRNVLELSNVVRSANYTCVAISSLGMIEATAQVTVKALPKPPIDLVVTETTATSVTLTWDSGNSEPVT
YYGIQYRAAGTEGPFQEVDGVATTRYSIGGLSPFSEYAFRVLAVNSIGRGPPSEAVRARTGEQAPSSPPRRVQARMLSASTMLVQWE
PPEEPNGLVRGYRVYYTPDSRRPPNAWHKHNTDAGLLTTVGSLLPGITYSLRVLAFTAVGDGPPSPTIQVKTQQGVPAQPADFQAEV
ESDTRIQLSWLLPPQERIIMYELVYWAAEDEDQQHKVTFDPTSSYTLEDLKPDTLYRFQLAARSDMGVGVFTPTIEARTAQSTPSAP
PQKVMCVSMGSTTVRVSWVPPPADSRNGVITQYSVAYEAVDGEDRORHVVDGISREHSSWDLVGLEKWTEYRVWVRAHTDVGPGPES
SPVLVRTDEDVPSOPPRKVEVEPLNSTAVHVYWKLPVPSKQHGQIRGYQVTYVRLENGEPRGLPIIQDVMLAEAQETTISGLTPETT
YSVTVAAYTTKGDGARSKPKIVTTTGAVPGRPTMMISTTANNTALLQWHPPKELPGELLGYRLQYCRADEARPNTIDFGKDDQHFTV
TGLHKGTTYIFRLAAKNRAGLGEEFEKEIRTPEDLPSGFPQNLHVTGLTTSTTELAWDPPVLAERNGRIISYTVVFRDINSQQELQN
ITTDTRFTLTGLKPDTTYDIKVRAWTSKGSGPLSPSIQSRTMPVEQVFAKNFRVAAANKTSVLLSWEVPDSYKSAVPFKILYNGQSV
EVDGHSMRKLIANLQPNTEYSFVLMNRGSSAGGLQHLVSIRTAPDLLPHKPLPASAYIEDGRFDLSMPHVQDPSLVRWFYIVVVPID
RVGGSMLTPRWSTPEELELDELLEAIEQGGEEQRRRRRQAERLKPYVAAQLDVLPETFTLGDKKNYRGFYNRPLSPDLSYQCFVLAS
LKEPMDQKRYASSPYSDEIVVQVTPAQQQEEPEMLWVTGPVLAVILIILIVIAILLFKRKRTHSPSSKDEQSIGLKDSLLAHSSDPV
EMRRLNYQTPGMRDHPPIPITDLADNIERLKANDGLKFSQEYESIDPGQQFTWENSNLEVNKPKNRYANVIAYDHSRVILTSIDGVP
GSDYINAYIDGYRKQNAYIATQGPLPETMGDFWRNVWEQRTATVVIMMTRLEEKSRVKCDQYWPARGTETCGLIQVTLLDTVELATY
TVRTFALHKSGSSEKRELRQFQFMAWPDHGVPEYPTPILAFLRRVKACNPLDAGPMVVHCSAGVGRTGCFIVIDANLERMKHEKTVD
IYGHTCMRSQRNYMVQTEDQYVFIHEALLEAATCGHTEVPARNNYLAHIQKLGQVPPGASVTANELEFKLLASSKAMTSRFISANLP
CNKFKNRLVNIMPYELTRVCLQPIRGVEGSDYINASFLDGYRQQKAYIATQGPLAESTEDFWRMLWEHNSTIIVHLTKLREMGREKC
HQYWPAERSARYQYFVDPMAEYNMPQYILREFKVTDARDGQSRTIRQFQPTDWPEQGVPKTGEGFIDFIGQAVHKTKEQFGQDGPIT
VHCSAGVGRTGVFITLSIVLERMRYEGVVDMFQTVKTLRTQRPAMVQTEDQYQLCYRAALEYLGSFDHYAT*
HUMAN mRNA SEQUENCE: hR28-021.3 (Seq ID No: 71)
GCGTGTGTGGACTGAAACCCAAAAGCCAGAAACTCTCTGCATGTGTCCCTCATGGAGCCATATGGTCTCTAGTCTCTGTTTTTCAAA
ATTTGCAGGACTGATGACTGATTGGACAGTGGTGCAGGAGGGAAGGTCTCTGCATGTCAGTGATTAAATTGCTGAAGATGCCACTGC
CTGAGACGGGCAGTATTGAAGGAAGGAGTGGAGGCCCTGGTGCCCGGCCCTTGGTGCTGAGTATCCAGCAAGAGTGACCGGGGTGAA
GAAGCAAAGACTCGGTTGATTGTCCTGGGCTGTGGCTGGCTGTGGAGCTAGAGCCCTGGATGGCCCCTGAGCCAGCCCCAGGGAGGA
CGATGGTGCCCCTTGTGCCTGCACTGGTGATGCTTGGTTTGGTGGCAGGCGCCCATGGTGACAGCAAACCTGTCTTCATTAAAGTCC
CTGAGGACCAGACTGGGCTGTCAGGAGGGGTAGCCTCCTTCGTGTGCCAAGCTACAGGAGAACCCAAGCCGCGCATCACATGGATGA
AGAAGGGGAAGAAAGTCAGCTCCCAGCGCTTCGAGGTCATTGAGTTTGATGATGGGGCAGGGTCAGTGCTTCGGATCCAGCCATTGC
GGGTGCAGCGAGATGAAGCCATCTATGAGTGTACAGCTACTAACAGCCTGGGTGAGATCAACACTAGTGCCAAGCTCTCAGTGCTCG
AAGAGGAACAGCTGCCCCCTGGGTTCCCTTCCATCGACATGGGGCCTCAGCTGAAGGTGGTGGAGAAGGCACGCACAGCCACCATGC
TATGTGCCGCAGGCGGAAATCCAGACCCTGAGATTTCTTGGTTCAAGGACTTCCTTCCTGTAGACCCTGCCACGAGCAACGGCCGCA
TCAAGCAGCTGCGTTCAGGTGCCTTGCAGATAGAGAGCAGTGAGGAATCCGACCAAGGCAAGTACGAGTGTGTGGCGACCAACTCGG
CAGGCACACGTTACTCAGCCCCTGCGAACCTGTATGTGCGAGTGCGCCGCGTGGCTCCTCGTTTCTCCATCCCTCCCAGCAGCCAGG
AGGTGATGCCAGGCGGCAGCGTGAACCTGACATGCGTGGCAGTGGGTGCACCCATGCCCTACGTGAAGTGGATGATGGGGGCCGAGG
AGCTCACCAAGGAGGATGAGATGCCAGTTGGCCGCAACGTCCTGGAGCTCAGCAATGTCGTACGCTCTGCCAACTACACCTGTGTGG
CCATCTCCTCGCTGGGCATGATCGAGGCCACAGCCCAGGTCACAGTGAAAGCTCTTCCAAAGCCTCCGATTGATCTTGTGGTGACAG
AGACAACTGCCACCAGTGTCACCCTCACCTGGGACTCTGGGAACTCGGAGCCTGTAACCTACTATGGCATCCAGTACCGCGCAGCGG
GCACGGAGGGCCCCTTTCAGGAGGTGGATGGTGTGGCCACCACCCGCTACAGCATTGGCGGCCTCAGCCCTTTCTCGGAATATGCCT
TCCGCGTGCTGGCGGTGAACAGCATCGGGCGAGGGCCGCCCAGCGAGGCAGTGCGGGCACGCACGGGAGAACAGGCGCCCTCCAGCC
CACCGCGCCGCGTGCAGGCACGCATGCTGAGCGCCAGCACCATGCTGGTGCAGTGGGAGCCTCCCGAGGAGCCCAACGGCCTGGTGC
GGGGATACCGCGTCTACTATACTCCGGACTCCCGCCGCCCCCCGAACGCCTGGCACAAGCACAACACCGACGCGGGGCTCCTCACGA
CCGTGGGCAGCCTGCTGCCTGGCATCACCTACAGCCTGCGCGTGCTTGCCTTCACCGCCGTGGGCGATGGCCCTCCCAGCCCCACCA
TCCAGGTCAAGACGCAGCAGGGAGTGCCTGCCCAGCCCGCGGACTTCCAGGCCGAGGTGGAGTCGGACACCAGGATCCAGCTCTCGT
GGCTGCTGCCCCCTCAGGAGCGGATCATCATGTATGAACTGGTGTACTGGGCGGCAGAGGACGAAGACCAACAGCACAAGGTGACCT
TCGACCCAACCTCCTCCTACACACTAGAGGACCTGAAGCCTGACACACTCTACCGCTTCCAGCTGGCTGCACGCTCGGATATGGGGG
TGGGCGTCTTCACCCCCACCATTGAGGCCCGCACAGCCCAGTCCACCCCCTCCGCCCCTCCCCAGAAGGTGATGTGTGTGAGCATGG
GCTCCACCACGGTCCGGGTAAGTTGGGTCCCGCCGCCTGCCGACAGCCGCAACGGCGTTATCACCCAGTACTCCGTGGCCTACGAGG
CGGTGGACGGCGAGGACCGCGGGCGGCATGTGGTGGATGGCATCAGCCGTGAGCACTCCAGCTGGGACCTGGTGGGCCTGGAGAAGT
GGACGGAGTACCGGGTGTGGGTGCGGGCACACACAGACGTGGGCCCCGGCCCCGAGAGCAGCCCGGTGCTGGTGCGCACCGATGAGG
ACGTGCCCAGCGGGCCTCCCCGGAAGGTGGAGGTGGAGCCACTGAACTCCACTGCTGTGCATGTCTACTGGAAGCTGCCTGTCCCCA
GCAAGCAGCATGGCCGGATCCGCGGCTACCAGGTCACCTACGTGCGGCTGGAGAATGGCGAGCCCCGTGGACTCCCCATCATCCAAG
ACGTCATGCTAGCCGAGGCCCAGGAAACCACTATCAGCGGCCTGACCCCGGAGACCACCTACTCCGTTACTGTTGCTGCCTATACCA
CCAAGGGGGATGGTGCCCGCAGCAAGCCCAAAATTGTCACTACAACAGGTGCAGTCCCAGGCCGGCCCACCATGATGATCAGCACCA
CGGCCATGAACACTGCGCTGCTCCAGTGGCACCCACCCAAGGAACTGCCTGGCGAGCTGCTGGGCTACCGGCTGCAGTACTGCCGGG
CCGACGAGGCGCGGCCCAACACCATAGATTTCGGCAAGGATGACCAGCACTTCACAGTCACCGGCCTGCACAAGGGGACCACCTACA
TCTTCCGGCTTGCTGCCAAGAACCGGGCTGGCTTGGGTGAGGAGTTCGAGAAGGAGATCAGGACCCCCGAGGACCTGCCCAGCGGCT
TCCCCCAAAACCTGCATGTGACAGGACTGACCACGTCTACCACAGAACTGGCCTGGGACCCGCCAGTGCTGGCGGAGAGGAACGGGC
GCATCATCAGCTACACCGTGGTGTTCCGAGACATCAACAGCCAACAGGAGCTGCAGAACATCACGACAGACACCCGCTTTACCCTTA
CTGGCCTCAAGCCAGACACCACTTACGACATCAAGGTCCGCGCATGGACCAGCAAAGGCTCTGGCCCACTCAGCCCCAGCATCCAGT
CCCGGACCATGCCGGTGGAGCAAGTGTTTGCCAAGAACTTCCGGGTGGCGGCTGCAATGAAGACGTCTGTGCTGCTCAGCTGGGAGG
TTCCCGACTCCTATAAGTCAGCTGTGCCCTTTAAGATTCTGTACAATGGGCAGAGTGTGGAGGTGGACGGGCACTCGATGCGGAAGC
TGATCGCAGACCTGCAGCCCAACACAGAGTACTCGTTTGTGCTGATGAACCGTGGCAGCAGCGCAGGGGGCCTGCAGCACCTGGTGT
CCATCCGCACAGCCCCCGACCTCCTGCCTCACAAGCCGCTGCCTGCCTCTGCCTACATAGAGGACGGCCGCTTCGATCTCTCCATGC
CCCATGTGCAAGACCCCTCGCTTGTCAGGTGGTTCTACATTGTTGTGGTACCCATTGACCGTGTGGGCGGGAGCATGCTGACGCCAA
GGTGGAGCACACCCGAGGAACTGGAGCTGGACGAGCTTCTAGAAGCCATCGAGCAAGGCGGAGAGGAGCAGCGGCGGCQGCGGCGGC
AGGCAGAACGTCTGAAGCCATATGTGGCTGCTCAACTGGATGTGCTCCCGGAGACCTTTACCTTGGGGGACAAGAAGAACTACCGGG
GCTTCTACAACCGGCCCCTGTCTCCGGACTTGAGCTACCAGTGCTTTGTGCTTGCCTCCTTGAAGGAACCCATGGACCAGAAGCGCT
ATGCCTCCAGCCCCTACTCGGATGAGATCGTGGTCCAGGTGACACCAGCCCAGCAGCAGGAGGAGCCGGAGATGCTGTGGGTGACGG
GTCCCGTGCTGGCAGTCATCCTCATCATCCTCATTGTCATCGCCATCCTCTTGTTCAAAAGGAAAAGGACCCACTCTCCGTCCTCTA
AGGATGAGCAGTCGATCGGACTGAAGGACTCCTTGCTGGCCCACTCCTCTGACCCTGTGGAGATGCGGAGGCTCAACTACCAGACCC
CAGGTATGCGAGACCACCCACCCATCCCCATCACCGACCTGGCGGACAACATCGAGCGCCTCAAAGCCAACGATGGCCTCAAGTTCT
CCCAGGAGTATGAGTCCATCGACCCTGGACAGCAGTTCACGTGGGAGAATTCAAACCTGGAGGTGAACAAGCCCAAGAACCGCTATG
CGAATGTCATCGCCTACGACCACTCTCGAGTCATCCTTACCTCTATCGATGGCGTCCCCGGGAGTGACTACATCAATGCCAACTACA
TCGATGGCTACCGCAAGCAGAATGCCTACATCGCCACGCAGGGCCCCCTGCCCGAGACCATGGGCGATTTCTGGAGAATGGTGTGGG
AACAGCGCACGGCCACTGTGGTCATGATGACACGGCTGGAGGAGAAGTCCCGGGTAAAATGTGATCAGTACTGGCCAGCCCGTGGCA
CCGAGACCTGTGGCCTTATTCAGGTGACCCTGTTGGACACAGTGGAGCTGGCCACATACACTGTGCGCACCTTCGCACTCCACAAGA
GTGGCTCCAGTGAGAAGCGTGAGCTGCGTCAGTTTCAGTTCATGGCCTGGCCAGACCATGGAGTTCCTGAGTACCCAACTCCCATCC
TGGCCTTCCTACGACCGGTCAAGGCCTGCAACCCCCTAGACGCAGGGCCCATGGTGGTGCACTGCAGCGCGGGCGTGGGCCGCACCG
GCTGCTTCATCGTGATTGATGCCATGTTGGAGCGGATGAAGCACGAGAAGACGGTGGACATCTATGGCCACGTGACCTGCATGCGAT
CACAGAGGAACTACATGGTGCAGACGGAGGACCAGTACGTGTTCATCCATGAGGCGCTGCTGGAGGCTGCCACGTGCGGCCACACAG
AGGTGCCTGCCCGCAACCTGTATGCCCACATCCAGAAGCTGGGCCAAGTGCCTCCAGGGGAGAGTGTGACCGCCATGGAGCTCGAGT
TCAAGTTGCTGGCCAGCTCCAAGGCCCACACGTCCCGCTTCATCAGCGCCAACCTGCCCTGCAACAAGTTCAAGAACCGGCTGGTGA
ACATCATGCCCTACGAATTGACCCGTGTGTGTCTGCAGCCCATCCGTGGTGTGGAGGGCTCTGACTACATCAATGCCAGCTTCCTGG
ATGGTTATAGACAGCAGAAGGCCTACATAGCTACACAGGGGCCTCTGGCAGAGAGCACCGAGGACTTCTGGCGCATGCTATGGGAGC
ACAATTCCACCATCATCGTCATCCTGACCAAGCTTCGGGAGATGGGCAGGGAGAAATGCCACCAGTACTGGCCAGCAGAGCGCTCTG
CTCGCTACCAGTACTTTGTTGTTGACCCGATGGCTGAGTACAACATGCCCCAGTATATCCTCCGTGAGTTCAAGGTCACGGATGCCC
GGGATGGGCAGTCAAGGACAATCCGGCAGTTCCAGTTCACAGACTGGCCAGAGCAGGGCGTGCCCAAGACAGGCGAGGGATTCATTG
ACTTCATCGGGCAGGTGCATAAGACCAAGGAGCAGTTTGGACAGGATGGGCCTATCACGGTGCACTGCAGTGCTGGCGTGGGCCGCA
CCGGGGTGTTCATCACTCTGAGCATCGTCCTGGAGCGCATGCGCTACGAGGGCGTGGTCGACATGTTTCAGACCGTGAAGACCCTGC
GTACACAGCGTCCTGCCATGGTGCAGACAGAGGACCAGTATCAGCTGTGCTACCGTGCGGCCCTGGAGTACCTCGGCAGCTTTGACC
ACTATGCAACGTAACTACCGCTCCCCTCTCCTCCGCCACCCCCGCCGTGGGGCTCCGGAGGGGACCCAGCTCCTCTGAGCCATACCG
ACCATCGTCCAGCCCTCCTACGCAGATGCTGTCACTGGCAGAGCACAGCCCACGGGGATCACAGCGTTTCAGGAACGTTGCCACACC
AATCAGAGAGCCTAGAACATCCCTGGGCAAGTGGATGGCCCAGCGGGCAGGCACTGTGGCCCTTCTGTCCACCAGACCCACCTGGAG
CCCGCTTCAAGCTCTCTGTTGCGCTCCCGCATTTCTCATGCTTCTTCTCATGGGGTGGGGTTGGGGCAAAGCCTCCTTTTTAATACA
TTAAGTGGGGTAGACTGAGGGATTTTAGCCTCTTCCCTCTGATTTTTCCTTTCGCGAATCCGTATCTGCAGAATGGGCCACTGTAGG
GGTTGGGGTTTATTTTGTTTTGTTTTTTTTTTTCTTGATTTGTATGACTTCTGCTGAAGGACAGAACATTGCCTTCCTCGTGCAGAG
CTGGGGCTGCCAGCCTGAGCGGAGGCTCGGCCGTGGGCCGGGAGGCAGTGCTGATCCGGCTGCTCCTCCAGCCCTTCAGACGAGATC
CTGTTTCAGCTAAATGCAGGGAAACTCAATGTTTTTTTAAGTTTTGTTTTCCCTTTAAAGCCTTTTTTTAGGCCACATTGACAGTGG
TGGGCGGGGAGAAGATAGGGAACACTCATCCCTGGTCGTCTATCCCAGTGTGTGTTTAACATTCACAGCCCAGAACCACAGATGTGT
CTGGGAGAGCCTGGCAAGGCATTCCTCATCACCATCGTGTTTGCAAAGGTTAAAACAACAAAAAACCAAAACAAAAATAAAAAACAA
AAAAAACAAAAAACCCAAGAAAAAAAAAAAGAGTCAGCCCTTGGCTTCTGCTTCAAACCCTCAAGAGGGGAAGCAACTCCGTGTGCC
TGGGGTTCCCGAGGGAGCTGCTGGCTGACCTGGGCCCACAGAGCCTGGCTTTGGTCCCCAGCATTGCAGTATGGTGTGGTGTTTGTA
GGCTGTGGGGTCTGGCTGTGTGGCCAAGGTGAATAGCACAGGTTAGGGTGTGTGCCACACCCCATGCACCTCAGGGCCAAGCGGGGG
CGTGGCTGGCCTTTCAGGTCCAGGCCAGTGGGCCTGGTAGCACATGTCTGTCCTCAGAGCAGGGGCCAGATGATTTTCCTCCCTGGT
TTGCAGCTGTTTTCAAGCCCCCGATAATCGCTCTTTTCCACTCCAAGATGCCCTCATAAACCAATGTGAGCAAGACTACTGGACTTC
TATCAATGGTACTCTAATCAGTCCTTATTATCCCAGCTTGCTGAGGGGCAGGGAGAGCGCCTCTTCCTCTGOGCAGCGCTATCTAGA
TAGGTAAGTGGGGGCGGGGAAGGGTGCATAGCTGTTTTAGCTGAGGGACGTGGTGCCGACGTCCCCAAACCTAGCTAGGCTAAGTCA
AGATCAACATTCCAGGGTTGGTAATGTTGGATGATGAAACATTCATTTTTACCTTGTGGATGCTAGTGCTGTAGAGTTCACTGTTGT
ACACAGTCTGTTTTCTATTTGTTAAGAAAAACTACAGCATCATTGCATAATTCTTGATGGTAATAAATTTGAATAATCAGATTTCTT
AC
HUMAN PROTEIN SEQUENCE: hP28-021.3 (Seq ID No: 72)
MAPEPAPGRTMVPLVPALVGLVAGAHGDSKPVFIKVPEDQTGLSGGVASFVCQATGEPKPRITWMKKGKKVSSQRPGVIAAEFDDGA
GSVLRIQPLRVQRDEAIYECTATNSLGEINTSAKLSVLEEEQLPPGFPSIDMGPQLKVVEKARTATMLCAAGGNPDPEISWFKDFLP
VDPATSNGRIKQLRSGALQIESSEESDQGKYECVATNSAGTRYSAPANLYVRVRRVAPRFSIPPSSQEVMPGGSVNLTCVAVGAPMP
YVKWMMGAEELTKEDEMPVGRNVLELSNVVRSANYTCVAISSLGMIEATAQVTVKALPKPPIDLVVTETTATSVTLTWDSGNSEPVT
YYGIQYRAAGTEGPFQEVDGVATTRYSIGGLSPFSEYAFRVLAVNSIGRGPPSEAVRARTGEQAPSSPPRRVQARMLSASTMLVQWE
PPEEPNGLVRGYRVYYTPDSRRPPNAWHKHNTDAGLLTTVGSLLPGITYSLRVLAFTAVGDGPPSPTIQVKTQQGVPAQPADFQAEV
ESDTRIQLSWLLPPQERIIMYELVYWAAEDEDQQHKVTFDPTSSYTLEDLKPDTLYRFQLAARSDMGVGVFTPTIEARTAQsTPSAP
PQKVNCVSMGSTTVRVSWVPPPADSRNGVITQYSVAYEAVDGEDRGRHVVDGISREHSSWDLVGLEKWTEYRVWVRAMTDVGPGPES
SPVLVRTDEDVPSGPPRKVEVEPLNSTAVHVYWKLPVPSKQNGQIRGYQVTYVRLENGEPRGLPIIQDVMLAEAQETTISGLTPETT
YSVTVAAYTTKGDGARSKPKIVTTTGAVPGRPTMMISTTANNTALLQWEPPKELPGELLGYRLQYCRADEARPNTIDFGKDDQHFTV
TGLHKGTTYIFRLAAKNRAGLGEEFEKEIRTPEDLPSGFPQNLHVTGLTTSTTELAWDPPVLAERNGRIISYTVVFRDINSQQELQN
ITTDTRFTLTGLKPDTTYDIKVRAWTSKGSGPLSPSIQSRTTPVEQVFAKNFRVAAANKTSVLLSWEVPDSYKSAVPFKILYNGQSV
EVDGHSMRKLIADLQPNTEYSFVLMNRGSSAGGLQHLVSIRTAPDLLPHKPLPASAYIEDGRFDLSMPHVQDPSLVRWFYIVVVPID
RVGGSMLTPRWSTPEELELDELLEAIEQGGEEQRRRRRQAERLKPYVAAQLDVLPETFTLGDKKNYRGFYNRPLSPDLSYQCFVLAS
LKEPMDQKRYASSPYSDEIVVQVTPAQQQEEPEMLWVTCPVLAVILIILIVIAILLFKKARTHSPSSKDEQSIGLKDSLLANSSDPV
EMRRLNYQTPGMRDHPPIPITDLADNIERLKANDGLKFSQEYESIDPGQQFTWENSNLEVNKPKNRYANVIAYDHSRVILTSIDGVP
GSDYINANYIDGYRKQNAYIATQGPLPETMGDFWRMVWEQRTATVVMNTRLEEKSRVKCDQYWPARGTETCGLIQVTLLDTVELATY
TVRTFALHKSGSSEKRELRQFQFMAWPDHGVPEYPTPILAFLRRVKACNPLDAGPMVVHCSAGVGRTGCFIVIDAMLERMKHEKTVD
IYGNVTCMRSQRNYMVQTEDQYVFIHEALLEAATCGHTEVPARNLYAHQKLGQVPPGESWTANELEFKLLASSKAIITSRFISANLP
CNKFKNRLVNIMPYELTRVCLQPIRGVEGSDYINASFLDGYRQQKAYIATQGPLAESTEDFWRNLWEHNSTIIVMLTKLREMGREKC
HQYWPAERSARYQYFVVDPMAEYNNPQYILREFKVTDARDGQSRTIRQFQFTDWPEQGVPKTGEGFIDFIGQVHKTKEQFGQDGPIT
VHCSAGVGRTGVPITLSIVLERMRYEGVVDMFQTVKTLRTQRPANVQTEDQYQLCYRAALEYLGSFDHYAT*
HUMAN mRNA SEQUENCE: hR28-021.4 (Seq ID No: 73)
GCGTGTGTGGACTGAAACCCAAAAGCCAGAAACTCTCTGCATGTGTCCCTCATGGAGCCATATGGTCTCTAGTCTCTGTTTTTCAAA
ATTTGCAGGACTGATGACTGATTGGACAGTGGTGCAGGAGGGAAGGTCTCTGCATGTCAGTGATTAAATTGCTGAAGATGCCACTGC
CTGAGACGGGCAGTATTGAAGGAAGGAGTGGAGGCCCTGGTGCCCGGCCCTTGGTGCTGAGTATCCAGCAAGAGTGACCGGGGTGAA
GAAGCAAAGACTCGGTTGATTGTCCTGGGCTGTGGCTGGCTGTGGAGCTAGAGCCCTGGATGGCCCCTGAGCCAGCCCCAGGGAGGA
CGATGGTGCCCCTTGTGCCTGCACTGGTGATGCTTGGTTTGGTGGCAGGCGCCCATGGTGACAGCAAACCTGTCTTCATTAAAGTCC
CTGAGGACCAGACTGGGCTGTCAGGAGGGGTAGCCTCCTTCGTGTGCCAAGCTACAGGAGAACCCAAGCCGCGCATCACATGGATGA
AGAAGGGGAAGAAAGTCAGCTCCCAGCGCTTCGAGGTCATTGAGTTTGATGATGGGGCAGGGTCAGTGCTTCGGATCCAGCCATTGC
GGGTCCAGCGAGATGAAGCCATCTATGAGTGTACAGCTACTAACAGCCTGGGTGAGATCAACACTAGTGCCAAGCTCTCAOTGCTCG
AAGAGGAACAGCTGCCCCCTGGGTTCCCTTCCATCGACATGGGGCCTCAGCTGAAGGTGGTGGAGAAGGCACGCACAGCCACCATGC
TATGTGCCGCAGGCGGAAATCCAGACCCTGAGATTTCTTGGTTCAAGGACTTCCTTCCTGTAGACCCTGCCACGAGCAACGGCCGCA
TCAAGCAGCTGCGTTCAGGTGCCTTGCAGATAGAGAGCAGTGAGGAATCCGACCAAGGCAAGTACGACTGTGTGGCGACCAACTCGG
CAGGCACACGTTACTCAGCCCCTGCGAACCTGTATGTGCGAGTGCGCCGCGTGGCTCCTCGTTTCTCCATCCCTCCCAGCAGCCAGG
AGGTGATGCCAGGCGGCAGCGTGAACCTGACATGCGTGGCAGTGGGTGCACCCATGCCCTACGTGAAGTGGATGATGGGGGCCGAGG
AGCTCACCAAGGAGGATGAGATGCCAGTTGGCCGCAACGTCCTGGAGCTCAOCAATGTCGTACGCTCTGCCAACTACACCTGTGTGG
CCATCTCCTCGCTGGGCATGATCGAGGCCACAGCCCAGGTCACAGTGAAAGCTCTTCCAAAGCCTCCGATTGATCTTGTGOTGACAG
AGACAACTGCCACCAGTGTCACCCTCACCTGGGACTCTGGGAACTCGGAGCCTGTAACCTACTATGGCATCCAGTACCGCGCAGCGG
GCACGGAGGGCCCCTTTCAGGAGGTGGATGGTGTGGCCACCACCCGCTACAGCATTGGCGGCCTCAGCCCTTTCTCGGAATATGCCT
TCCGCGTGCTGGCGGTGAACAGCATCGGGCGAGGGCCGCCCAGCGAGGCAGTGCGGGCACGCACGGGAGAACAGGCGCCCTCCAGCC
CACCGCGCCGCGTGCAGGCACGCATGCTGAGCGCCAGCACCATGCTGGTGCAGTGGGAGCCTCCCGAGGAGCCCCCCGGCCTGGTGC
GGGGATACCGCGTCTACTATACTCCGGACTCCCGCCGCCCCCCGAACGCCTGGCACAAGCACAACACCGACGCGGGGCTCCTCACGA
CCGTGGGCAGCCTGCTGCCTGGCATCACCTACAGCCTGCGCGTGCTTGCCTTCACCGCCGTGGGCGATGGCCCTCCCAGCCCCACCA
TCCAGGTCAAGACGCAGCAGGGAGTGCCTCCCCAGCCCGCGGACTTCCAGGCCGAGGTGGAGTCGGACACCAGGATCCAGCTCTCGT
GGCTGCTGCCCCCTCAGGAGCGGATCATCATGTATGAACTGGTGTACTGGGCGGCAGAGGACGAAGACCAACAGCACAAGGTGACCT
TCGACCCAACCTCCTCCTACACACTAGAGGACCTGAAGCCTGACACACTCTACCGCTTCCAGCTGGCTGCACGCTCGGATATGGGGG
TGGGCGTCTTCACCCCCACCATTGAGGCCCGCACAGCCCAGTCCACCCCCTCCGCCCCTCCCCAGAAGGTGATGTGTGTGAGCATGG
GCTCCACCACGGTCCGGGTAAGTTGGGTCCCGCCGCCTGCCGACAGCCGCAACGGCGTTATCACCCAGTACTCCGTGGCCTACGAGG
CGGTGGACGGCGAGGACCGCGGGCGGCATGTGGTGGATGGCATCAGCCGTGAGCACTCCAGCTGGGACCTGGTGGGCCTGGAGAAGT
GGACGGAGTACCGGGTGTGGGTGCGGGCACACACAGACGTGGGCCCCGGCCCCGAGAGCAGCCCGGTGCTGGTGCGCACCGATGAGG
ACGTGCCCAGCGGGCCTCCGCGGAAGGTGGAGGTGGAGCCACTGAACTCCACTGCTGTGCATGTCTACTGGAAGCTGCCTGTCCCCA
GCAAGCAGCATGGCCAGATCCGCGGCTACCAGGTCACCTACGTGCGGCTGGAGAATGGCGAGCCCCGTGGACTCCCCATCATCCAAG
ACGTCATGCTAGCCGAGGCCCAGTGGCGGCCAGAGGACTCCGAGGACTATGAAACCACTATCAGCGGCCTGACCCCGGAGACCACCT
ACTCCGTTACTGTTGCTGCCTATACCACCAAGGGGGATGGTGCCCGCAGCAAGCCCAAAATTGTCACTACAACAGGTGCAGTCCCAG
GCCGGCCCACCATGATGATCAGCACCACGCCCATGAACACTGCGCTGCTCCAGTGGCACCCACCCAAGGAACTGCCTGGCGAGCTGC
TGGGCTACCGGCTGCAGTACTGCCGGGCCGACGAGGCGCGGCCCAACACCATAGATTTCGGCAAGGATGACCAGCACTTCACAGTCA
CCGGCCTGCACAAGGGGACCACCTACATCTTCCGGCTTGCTGCCAAGAACCGGGCTGGCTTGGGTGAGGAGTTCGAGAAGGAGATCA
GGACCCCCGAGGACCTGCCCAGCGGCTTCCCCCAAAACCTGCATGTGACAGGACTGACCACGTCTACCACAGAACTGGCCTGGGACC
CGCCAGTGCTGGCGGAGAGGAACGGGCGCATCATCAGCTACACCGTGGTGTTCCGAGACATCAACAGCCAACAGGAGCTGCAGAACA
TCACGACAGACACCCGCTTTACCCTTACTGGCCTCAAGCCAGACACCACTTACGACATCAAGGTCCGCGCATGGACCAGCAAAGGCT
CTGGCCCACTCAGCCCCAGCATCCAGTCCCGGACCATGCCGGTGGAGCAAGTGTTTGCCAAGAACTTCCGGGTGGCGGCTGCAATGA
AGACGTCTGTGCTGCTCAGCTGGGAGGTTCCCGACTCCTATAAGTCAGCTGTGCCCTTTAAGATTCTGTACAATGGGCAGAGTGTGG
AGGTGGACGGGCACTCGATGCGGAAGCTGATCGCAGACCTGCAGCCCAACACAGAGTACTCGTTTGTGCTGATGAACCGTGGCAGCA
GCGCAGGGGGCCTGCAGCACCTGGTGTCCATCCGCACAGCCCCCGACCTCCTGCCTCACAAGCCGCTGCCTGCCTCTGCCTACATAG
AGGACGGCCGCTTCGATCTCTCCATGCCCCATGTGCAAGACCCCTCGCTTGTCAGGTGGTTCTACATTGTTGTGGTACCCATTGACC
GTGTGGGCGGGAGCATGCTGACGCCAAGGTGGAGCACACCCGAGGAACTGGAGCTGGACGAGCTTCTAGAAGCCATCGAGCAAGGCG
GAGAGGAGCAGCGGCGGCGGCGGCGGCAGGCAGAACGTCTGAAGCCATATGTGGCTGCTCAACTGGATGTGCTCCCGGAGACCTTTA
CCTTGGGGGACAAGAAGAACTACCGGGGCTTCTACAACCGGCCCCTGTCTCCGGACTTGAGCTACCAGTGCTTTGTGCTTGCCTCCT
TGAAGGAACCCATGGACCAGAAGCGCTATGCCTCCAGCCCCTACTCGGATGAGATCGTGGTCCAGGTGACACCAGCCCAGCAGCAGG
AGGAGCCGGAGATGCTGTGGGTGACGGGTCCCGTGCTGGCAGTCATCCTCATCATCCTCATTGTCATCGCCATCCTCTTGTTCAAAA
GGAAAAGGACCCACTCTCCGTCCTCTAAGGATGAGCAGTCGATCGGACTGAAGGACTCCTTGCTGGCCCACTCCTCTGACCCTGTGG
AGATGCGGAGGCTCAACTACCAGACCCCAGGTATGCGAGACCACCCACCCATCCCCATCACCGACCTGGCGGACAACATCGAGCGCC
TCAAAGCCAACGATGGCCTCAAGTTCTCCCAGGAGTATGAGTCCATCGACCCTGGACAGCAGTTCACGTGGGAGAATTCAAACCTGG
AGGTGAACAAGCCCAAGAACCGCTATGCGAATGTCATCGCCTACGACCACTCTCGAGTCATCCTTACCTCTATCGATGGCGTCCCCG
GGAGTGACTACATCAATGCCAACTACATCGATGGCTACCGCAAGCAGAATGCCTACATCGCCACGCAGGGCCCCCTGCCCGAGACCA
TGGGCGATTTCTGGAGAATGGTGTGGGAACAGCGCACGGCCACTGTGGTCATGATGACACGGCTGGAGGAGAAGTCCCGGGTAAAAT
GTGATCAGTACTGGCCAGCCCGTGGCACCGAGACCTGTGGCCTTATTCAGGTGACCCTGTTGGACACAGTGGAGCTGGCCACATACA
CTGTGCGCACCTTCGCACTCCACAAGAGTGGCTCCAGTGAGAAGCGTGACCTGCGTCAGTTTCAGTTCATGGCCTGGCCAGACCATG
GAGTTCCTGAGTACCCAACTCCCATCCTGGCCTTCCTACGACGGGTCAAGGCCTGCAACCCCCTAGACGCAGGGCCCATGGTGGTGC
ACTGCAGCGCGGGCGTGGGCCGCACCGGCTGCTTCATCGTGATTGATGCCATGTTGGAGCGGATGAAGCACGAGAAGACGGTGGACA
TCTATGGCCACGTGACCTGCATGCGATCACAGAGGAACTACATGGTGCAGACGGAGGACCAGTACGTGTTCATCCATGAGGCGCTGC
TGGAGGCTGCCACGTGCGGCCACACAGAGGTGCCTGCCCGCAACCTGTATGCCCACATCCAGAAGCTGGGCCAAGTGCCTCCAGGGG
AGAGTGTGACCGCCATGGAGCTCGAGTTCAAGTTGCTGGCCAGCTCCAAGGCCCACACGTCCCGCTTCATCAGCGCCAACCTGCCCT
GCAACAAGTTCAAGAACCGGCTGGTGAACATCATGCCCTACGAATTGACCCGTGTGTGTCTGCAGCCCATCCGTGGTGTGGAGGGCT
CTGACTACATCAATGCCAGCTTCCTGGATGGTTATAGACAGCAGAAGGCCTACATAGCTACACAGGGGCCTCTGGCAGAGAGCACCG
AGGACTTCTGGCGCATGCTATGGGAGCACAATTCCACCATCATCGTCATGCTGACCAAGCTTCGGGAGATGGGCAGGGAGAAATGCC
ACCAGTACTGGCCAGCAGAGCGCTCTGCTCGCTACCAGTACTTTGTTGTTGACCCGATGGCTGAGTACAACATGCCCCAGTATATCC
TGCGTGAGTTCAAGGTCACGGATGCCCGGGATGGGCAGTCAAGGACAATCCGGCAGTTCCAGTTCACAGACTGGCCAGAGCAGGGCG
TGCCCAAGACAGGCGAGGGATTCATTGACTTCATCGGGCAGGTGCATAAGACCAAGGAGCAGTTTGGACAGGATGGGCCTATCACGG
TGCACTGCAGTGCTGGCGTGGGCCGCACCGGGGTGTTCATCACTCTGAGCATCGTCCTGGAGCGCATGCGCTACGAGGGCGTGGTCG
ACATGTTTCAGACCGTGAAGACCCTGCGTACACAGCGTCCTGCCATGGTGCAGACAGAGGACCAGTATCAGCTGTGCTACCGTGCGG
CCCTGGAGTACCTCGGCAGCTTTGACCACTATGCAACGTAACTACCGCTCCCCTCTCCTCCGCCACCCCCGCCGTGGGGCTCCGGAG
GGACCCAGCTCCTCTGAGCCATACCGACCATCGTCCAGCCCTCCTACGCAGATGCTGTCACTGGCAGAGCACAGCCCACGGGAGATC
ACAGCGTTTCAGGAACGTTGCCACACCAATCAGAGAGCCTAGAACATCCCTGGGCAAGTGGATGGCCCAGCAGGCAGGCACTGTGGC
CCTTCTGTCCACCAGACCCACCTGGAGCCCGCTTCAAGCTCTCTGTTGCGCTCCCGCATTTCTCATGCTTCTTCTCATGGGGTGGGG
TTGGGGCAAAGCCTCCTTTTTAATACATTAAGTGGGGTAGACTGAGGGATTTTAGCCTCTTCCCTCTGATTTTTCCTTTCGCGAATC
CGTATCTGCAGAATGGGCCACTGTAGGGGTTGGGGTTTATTTTGTTTTGTTTTTTTTTTTCTTGATTTGTATGACTTCTGCTGAAGG
ACAGAACATTGCCTTCCTCGTGCAGAGCTGGGGCTGCCAGCCTGAGCGGAGGCTCGGCCGTGGGCCGGGAGGCAGTGCTGATCCGGC
TGCTCCTCCAGCCCTTCAGACGAGATCCTGTTTCAGCTAAATGCAGGGAAACTCAATGTTTTTTTAAGTTTTGTTTTCCCTTTAAAG
CCTTTTTTTAGGCCACATTGACAGTGGTGGGCGGGGAGAAGATAGGGAACACTCATCCCTGGTCGTCTATCCCAGTGTGTGTTTAAC
ATTCACAGCCCAGAACCACAGATGTGTCTGGGAGAGCCTGGCAAGGCATTCCTCATCACCATCGTGTTTGCAAAGGTTAAAACAAAA
ACAAAAAACCACAAAAATAAAAAACAAAAAAAACAAAAAACCCAAGAAAAAAAAAAAGAGTCAGCCCTTGGCTTCTGCTTCAAACCC
TCAAGAGGGGAAGCAACTCCGTGTGCCTGGGGTTCCCGAGGGAGCTGCTGCCTGACCTGGGCCCACAGAGCCTGGCTTTGGTCCCCA
GCATTGCAGTATGGTGTGGTGTTTGTAGGCTGTGGGGTCTGGCTGTGTGGCCAAGGTGAATAGCACAGGTTAGGGTGTGTGCCACAC
CCCATGCACCTCAGCGCCAAGCGGGGGCGTGGCTGGCCTTTCAGGTCCAGGCCAGTGGGCCTGGTAGCACATGTCTGTCCTCAGAGC
AGGGGCCAGATGATTTTCCTCCCTGGTTTGCAGCTGTTTTCAAAGCCCCCGATAATCGCTCTTTTCCACTCCAAGATGCCCTCATAA
ACCAATGTGGCAAGACTACTGGACTTCTATCAATGGTACTCTAATCAGTCCTTATTATCCCAGCTTGCTGAGGGGCAGGGAGAGCGC
CTCTTCCTCTGGGCAGCGCTATCTAGATAGGTAAGTGGGGGCGGGGAAGGGTGCATAGCTGTTTTAGCTGAGGGACGTGGTGCCGAC
GTCCCCAAACCTAGCTAGGCTAAGTCAAGATCAACATTCCAGGGTTGGTAATGTTGGATGATGAAACATTCATTTTTACCTTGTGGA
TGCTAGTGCTGTAGAGTTCACTGTTGTACACAGTCTGTTTTCTATTTGTTAAGAAAAACTACAGCATCATTGCATAATTCTTGATGG
TAATAAATTTGAATAATCAGATTTCTTAC
HUMAN PROTEIN SEQUENCE hP28-021.4 (Seq ID No: 74)
MAPEPAPGRTMVPLVPALVMLGLVAGAHGDSKPVFKVPEDQTGLSGGVASFVCQATGEPKPRITWMKKGKKVSSQRFEVIAEFDDGA
GSVLRIQPLRVQRDEAIYECTATNSLGEINTSAKLSVLEEEQLPPGFPSIDMGPQLKVVEKARTATMLCAAGGNPDPEISWFKDFLP
VDPATSNGRIKQLRSGALQIESSEESDQGKYECVATNSAGTRYSAPANLYVRVRRVAPRFSIPPSSQEVMPGGSVNLTCVAVGAPMP
YVKWMGAEELTKEDEMPVGRNVLELSNVVPSANYTCVAISSLGMIEATAQVTVKALPKPPIDLVVTETTATSVTLTAWDSGNSEPVT
YYGIQYRAAGTEGPFQEVDGVATTRYSIGGLSPFSEYAFRVLAVNSIGRGPPSEAVRARTGEQAPSSPPRRVQARMLSASTMLVQWE
PPEEPNGLVRGYNVYYTPDSRRPPMAWHKHNTDAGLLTTVGSLLPGITYSLRVLAFTAVGDGPPSPTIQVKTQQGVPAQPADFQAEV
ESDTRIQLSWLLPPQERIIMYELVYWAAEDEDQQHKVTFDPTSSYTLEDLKPDTLYRFQLAARSDMGVGVFTPTIEARTAQSTPSAP
PQKVMCVSMGSTTVRVSWVPPPADSRNGVITQYSVAYEAVDGEDRGRHVVDGISREHSSWDLVGLEKWTEYRVWVRAHTDVGPGPES
SPVLVRTDEDVPSGPPRKVEVEPLNSTAVHVYWKLPVPSKQHGQIRGYQVTYVRLNGEPRGLPIIQDVHMLAEAQWRPEESEDYETT
ISGLTPETTYSVTVAAYTTKGDGARSKPKIVTTTGAVPGRPTMMISTTANNTALLQWHPPKELPGELLGYRLQYCRADEARPNTIDF
GKDDQHFTVTGLNKGTTYIFRLAANRAGLGEEFEKEIRTPAADLPSGFPQNLHVTGLTTSTTALAWDPPVLAERNGRIISYTVVFRD
INSQQELQNITTDTRFTLTGLKPDTTYDIKVRAWTSKGSGPLSPSIQSRTMPVEQVFAKNFRVAAAMKTSVLLSWEVPDSYKSAVPF
KILYNGQSVEVDGHSMRKLIANLQPNTEYSFVLMNRGSSAGGLQHLVSIRTAPDLLPHKPLPASAYIEDGRFDLSTPHVQDPSLVRW
FYIVVVPIDRVGGSMLTPRWSTPEELELDELLEAIEQGGEEQRRRRRQANRLKPYVAAQLDVLPETFTLGDKKNYRGFYNRPLSPDL
SYQCFVLASLKEPMDQKRYASSPYSDEIVVQVTPAQQQEEPEMLWVTGPVLAVILIILIVIAILLFKRKRTHSPSSKDAQSIGLKDS
LLAHSSDPVEMRRLNYQTPGMRNHPPIPUTDLADNIERLKANDGLKFSQEYESIDPGQQFTWENSNLEVNKPKNRYANVIAYDHSRV
ILTSIDGVPGSDYINANYIDGYRKQNAYIATQGPLPETMGDFWRMVWEQRTATVVMMTRLEEKSRVKCDQYWPARGETCGLAIQVTL
LDTVELATYTVRTFALHKSGSSEKRELRQFQFMAWPDHGVPEYPTPILAFLRRVKACNPLDAGPMVVHCSAGVGRTGCFIVIDANLE
RMKHEKTVDIYGHVTCMRSQRNYNVQTEDQYVFIHEALLEAATCGHTEVPARNLYAHIQKLGQVPPGESVTAMELEFKLLASSKAHT
SRFISANLPCNKFKNRLVNIMPYELTRVCLQPIRGVEGSDYINASFLDGYRQQKAYIATQGPLAESTEDFWRMLWEHNSTIIVMLTK
LREMGREKCHQYWPAERSARYQYFVVDPMAEYNMPQYILREFKVTDARDGQSRTIRQFQFTDWPEQGVPKTGEGFIDFIGQVMKTKE
QFGQDGPITVHCSAGVGRTGVFITLSIVLERMRYEGVVDMFQTVKTLRTQRPANVQTEDQYQLCYRAALEYLGSFDHYAT*

The mouse and human genomic DNA sequence, sequence corresponding to the mRNA(s) generated therefrom and amino acid sequences of the proteins as shown in Tables 1-7 are described according to SEQ ID NOS as follows in Table 8.

TABLE 8

| DESIGNATION | SEQ ID NO | TYPE OF SEQUENCE |
|---|---|---|
| mD22-022 | SEQ ID NO: 1 | MOUSE GENOMIC SEQUENCE |
| mR22-022.1 | SEQ ID NO: 2 | MOUSE mRNA SEQUENCE |
| mP22-022.1 | SEQ ID NO: 3 | MOUSE PROTEIN SEQUENCE |
| mR22-022.2 | SEQ ID NO: 4 | MOUSE mRNA SEQUENCE |
| mP22-022.2 | SEQ ID NO: 5 | MOUSE PROTEIN SEQUENCE |
| hD22-022 | SEQ ID NO: 6 | HUMAN GENOMIC SEQUENCE |
| hR22-022.1 | SEQ ID NO: 7 | HUMAN mRNA SEQUENCE |
| hP22-022.1 | SEQ ID NO: 8 | HUMAN PROTEIN SEQUENCE |
| hR22-022.2 | SEQ ID NO: 9 | HUMAN mRNA SEQUENCE |
| hP22-022.2 | SEQ ID NO: 10 | HUMAN PROTEIN SEQUENCE |
| mD27-007 | SEQ ID NO: 11 | MOUSE GENOMIC SEQUENCE |
| mR27-007.1 | SEQ ID NO: 12 | MOUSE mRNA SEQUENCE |
| mP27-007.1 | SEQ ID NO: 13 | MOUSE PROTEIN SEQUENCE |
| mR27-007.2 | SEQ ID NO: 14 | MOUSE mRNA SEQUENCE |
| mP27-007.2 | SEQ ID NO: 15 | MOUSE PROTEIN SEQUENCE |
| hD27-007 | SEQ ID NO: 16 | HUMAN GENOMIC SEQUENCE |
| hR27-007.1 | SEQ ID NO: 17 | HUMAN mRNA SEQUENCE |
| hP27-007.1 | SEQ ID NO: 18 | HUMAN PROTEIN SEQUENCE |
| hR27-007.2 | SEQ ID NO: 19 | HUMAN mRNA SEQUENCE |
| hP27-007.2 | SEQ ID NO: 20 | HUMAN PROTEIN SEQUENCE |
| hR27-007.3 | SEQ ID NO: 21 | HUMAN mRNA SEQUENCE |
| hP27-007.3 | SEQ ID NO: 22 | HUMAN PROTEIN SEQUENCE |
| mD27-009 | SEQ ID NO: 23 | MOUSE GENOMIC SEQUENCE |
| mR27-009.1 | SEQ ID NO: 24 | MOUSE mRNA SEQUENCE |
| mP27-009.1 | SEQ ID NO: 25 | MOUSE PROTEIN SEQUENCE |
| hD27-009 | SEQ ID NO: 26 | HUMAN GENOMIC SEQUENCE |
| hR27-009.1 | SEQ ID NO: 27 | HUMAN mRNA SEQUENCE |
| hP27-009.1 | SEQ ID NO: 28 | HUMAN PROTEIN SEQUENCE |
| hR27-009.2 | SEQ ID NO: 29 | HUMAN mRNA SEQUENCE |
| hP27-009.2 | SEQ ID NO: 30 | HUMAN PROTEIN SEQUENCE |
| mD27-014 | SEQ ID NO: 31 | MOUSE GENOMIC SEQUENCE |
| mR27-014.1 | SEQ ID NO: 32 | MOUSE mRNA SEQUENCE |
| mP27-014.1 | SEQ ID NO: 33 | MOUSE PROTEIN SEQUENCE |
| mR27-014.2 | SEQ ID NO: 34 | MOUSE mRNA SEQUENCE |
| mP27-014.2 | SEQ ID NO: 35 | MOUSE PROTEIN SEQUENCE |
| mR27-014.3 | SEQ ID NO: 36 | MOUSE mRNA SEQUENCE |
| mP27-014.3 | SEQ ID NO: 37 | MOUSE PROTEIN SEQUENCE |
| mR27-014.4 | SEQ ID NO: 38 | MOUSE mRNA SEQUENCE |
| mP27-014.4 | SEQ ID NO: 39 | MOUSE PROTEIN SEQUENCE |
| hD27-014 | SEQ ID NO: 40 | HUMAN GENOMIC SEQUENCE |
| hR27-014.1 | SEQ ID NO: 41 | HUMAN mRNA SEQUENCE |
| hP27-014.1 | SEQ ID NO: 42 | HUMAN PROTEIN SEQUENCE |
| hR27-014.2 | SEQ ID NO: 43 | HUMAN mRNA SEQUENCE |
| hP27-014.2 | SEQ ID NO: 44 | HUMAN PROTEIN SEQUENCE |
| hR27-014.3 | SEQ ID NO: 45 | HUMAN mRNA SEQUENCE |
| hP27-014.3 | SEQ ID NO: 46 | HUMAN PROTEIN SEQUENCE |
| hR27-014.4 | SEQ ID NO: 47 | HUMAN mRNA SEQUENCE |
| hP27-014.4 | SEQ ID NO: 48 | HUMAN PROTEIN SEQUENCE |
| mD28-002 | SEQ ID NO: 49 | MOUSE GENOMIC SEQUENCE |
| mR28-002.1 | SEQ ID NO: 50 | MOUSE mRNA SEQUENCE |
| mP28-002.1 | SEQ ID NO: 51 | MOUSE PROTEIN SEQUENCE |
| hD28-002 | SEQ ID NO: 52 | HUMAN GENOMIC SEQUENCE |
| hR28-002.1 | SEQ ID NO: 53 | HUMAN mRNA SEQUENCE |
| hP28-002.1 | SEQ ID NO: 54 | HUMAN PROTEIN SEQUENCE |
| mD28-010 | SEQ ID NO: 55 | MOUSE GENOMIC SEQUENCE |
| mR28-010.1 | SEQ ID NO: 56 | MOUSE mRNA SEQUENCE |
| mP28-010.1 | SEQ ID NO: 57 | MOUSE PROTEIN SEQUENCE |
| mR28-010.2 | SEQ ID NO: 58 | MOUSE mRNA SEQUENCE |
| mP28-010.2 | SEQ ID NO: 59 | MOUSE PROTEIN SEQUENCE |
| hD28-010 | SEQ ID NO: 60 | HUMAN GENOMIC SEQUENCE |
| hR28-010.1 | SEQ ID NO: 61 | HUMAN mRNA SEQUENCE |
| hP28-010.1 | SEQ ID NO: 62 | HUMAN PROTEIN SEQUENCE |
| mD28-021 | SEQ ID NO: 63 | MOUSE GENOMIC SEQUENCE |
| mR28-021.1 | SEQ ID NO: 64 | MOUSE mRNA SEQUENCE |
| mP28-021.1 | SEQ ID NO: 65 | MOUSE PROTEIN SEQUENCE |
| hD28-021 | SEQ ID NO: 66 | HUMAN GENOMIC SEQUENCE |
| hR28-021.1 | SEQ ID NO: 67 | HUMAN mRNA SEQUENCE |
| hP28-021.1 | SEQ ID NO: 68 | HUMAN PROTEIN SEQUENCE |
| hR28-021.2 | SEQ ID NO: 69 | HUMAN mRNA SEQUENCE |
| hP28-021.2 | SEQ ID NO: 70 | HUMAN PROTEIN SEQUENCE |
| hR28-021.3 | SEQ ID NO: 71 | HUMAN mRNA SEQUENCE |
| hP28-021.3 | SEQ ID NO: 72 | HUMAN PROTEIN SEQUENCE |
| hR28-021.4 | SEQ ID NO: 73 | HUMAN mRNA SEQUENCE |
| hP28-021.4 | SEQ ID NO: 74 | HUMAN PROTEIN SEQUENCE |

The CA sequences were analyzed by Panther™ (Molecular Diagnostics, Palo Alto, Calif.) software designed to detect homologs and enable prediction of molecular function through a system for protein functional classification. Human Gene Ontlogy annotations were prepared in accordance with the Gene Ontology Consortium (Gene Ontology: tool for the unification of biology. The Gene Ontology Consortium Nature Genet. 25: 25-29 (2000)). Similar analysis was carried out by determining IPR information regarding the CA polypeptides from InterPro, which is an integrated documentation resource for protein families, domains and functional sites (Apweiler at al. Bioinformatics 16(12):1145-1150 (2000)).

The CA sequences may be classified according to the following predicted general classifications of function by Panther™ analysis, human gene ontology and IPR domain information for polypeptides having SEQ ID NOS: 8, 10, 18, 20, 22, 28, 30, 42, 44, 46, 48, 54, 62, 68, 70, 72 and 74 as shown in Tables 1-7. The classifications are shown in Table 9 below. The biological function of a CA protein comprises a function shown in Table 9 below.

TABLE 9

| Human Protein | SEQ ID NO: | FUNCTION |
|---|---|---|
| hP22-022.1 | SEQ ID NO: 8 | HUMAN PANTHER CLASSIFICATIONS |
| | | FAMILY (SUBFAMILY) |
| | | SHORT-CHAIN DEHYDROGENASE- |
| | | RELATED(FOLLICULAR VARIANT |
| | | TRANSLOCATION PROTEIN 1) |
| | | BIOLOGICAL PROCESS |
| | | Biological process |
| | | unclassified(2.99.00.00.00) |
| | | MOLECULAR FUNCTIONS |
| | | Oxidoreductase(1.19.00.00.00) > Reductase |
| | | (1.19.06.00.00) |
| | | Oxidoreductase(1.19.00.00.00) > Dehydrogenase |
| | | (1.19.05.00.00) |
| | | HUMAN GENE ONTOLOGY |
| | | No Gene Ontology |
| | | HUMAN PROTEIN DOMAINS (INTERPRO |
| | | SIGNATURES) |
| | | IPR002198 (SDRFAMILY) |
| | | IPR002347 (GDHRDH) |
| | | IPR002198 (adh short) |
| hP22-022.2 | SEQ ID NO: 10 | HUMAN PANTHER CLASSIFICATIONS |
| | | FAMILY (SUBFAMILY) |
| | | SHORT-CHAIN DEHYDROGENASE- |
| | | RELATED(FOLLICULAR VARIANT |
| | | TRANSLOCATION PROTEIN 1) |
| | | BIOLOGICAL PROCESS |
| | | Biological process |
| | | unclassified(2.99.00.00.00) |
| | | MOLECULAR FUNCTIONS |
| | | Oxidoreductase(1.19.00.00.00) > Reductase |
| | | (1.19.06.00.00) |
| | | Oxidoreductase(1.19.00.00.00) > Dehydrogenase |
| | | (1.19.05.00.00) |
| | | HUMAN GENE ONTOLOGY |
| | | No Gene Ontology |
| | | HUMAN PROTEIN DOMAINS (INTERPRO |
| | | SIGNATURES) |
| | | IPR002198 (SDRFAMILY) |
| | | IPR002347 (GDHRDH) |
| | | IPR002198 (adh short) |
| hP27-007.1 | SEQ ID NO: 18 | HUMAN PANTHER CLASSIFICATIONS |
| | | FAMILY (SUBFAMILY) |
| | | PLATELET ENDOTHELIAL CELL |
| | | ADHESION MOLECULE (PECAM-1)(SH2 |
| | | DOMAIN-CONTAINING PHOSPHATASE |
| | | ANCHOR PROTEIN 1-RELATED) |
| | | BIOLOGICAL PROCESS |
| | | Biological process |
| | | unclassified(2.99.00.00.00) |
| | | MOLECULAR FUNCTIONS |
| | | Molecular function |
| | | unclassified(1.97.00.00.00) |
| | | HUMAN GENE ONTOLOGY |
| | | BIOLOGICAL PROCESS |
| | | phagocytosis > phagocytosis, engulfment |
| | | defence response > immune response |
| | | MOLECULAR FUNCTION |
| | | GO molecular function > cell adhesion |
| | | defense/immunity protein > immunoglobulin |
| | | B cell receptor > immunoglobulin |
| | | CELL COMPONENT |
| | | cell > membrane fraction |
| | | cell > plasma membrane |
| | | HUMAN PROTEIN DOMAINS (INTERPRO |
| | | SIGNATURES) |
| | | IPR003598 (IGc2) |
| | | IPR003599 (IG) |
| | | IPR003006 (ig) |
| hP27-007.2 | SEQ ID NO: 20 | HUMAN PANTHER CLASSIFICATIONS |
| | | FAMILY (SUBFAMILY) |
| | | PLATELET ENDOTHELIAL CELL |
| | | ADHESION MOLECULE (PECAM-1)(SH2 |
| | | DOMAIN-CONTAINING PHOSPHATASE |
| | | ANCHOR PROTEIN 1-RELATED) |
| | | BIOLOGICAL PROCESS |
| | | Biological process |
| | | unclassified(2.99.00.00.00) |
| | | MOLECULAR FUNCTIONS |
| | | Molecular function |
| | | unclassified(1.97.00.00.00) |
| | | HUMAN GENE ONTOLOGY |
| | | BIOLOGICAL PROCESS |
| | | defence response > immune response |
| | | phagocytosis > phagocytosis, engulfment |
| | | MOLECULAR FUNCTION |
| | | defense/immunity protein > immunoglobulin |
| | | B cell receptor > immunoglobulin |
| | | GO molecular function > cell adhesion |
| | | CELL COMPONENT |
| | | cell > membrane fraction |
| | | cell > plasma membrane |
| | | HUMAN PROTEIN DOMAINS (INTERPRO |
| | | SIGNATURES) |
| | | IPR003598 (IGc2) |
| | | IPR003599 (IG) |
| | | IPR003006 (ig) |
| hP27-007.3 | SEQ ID NO: 22 | HUMAN PANTHER CLASSIFICATIONS |
| | | FAMILY (SUBFAMILY) |
| | | PLATELET ENDOTHELIAL CELL |
| | | ADHESION MOLECULE (PECAM-1)(SH2 |
| | | DOMAIN-CONTAINING PHOSPHATASE |
| | | ANCHOR PROTEIN 1-RELATED) |
| | | BIOLOGICAL PROCESS |
| | | Biological process |
| | | unclassified(2.99.00.00.00) |
| | | MOLECULAR FUNCTIONS |
| | | Molecular function |
| | | unclassified(1.97.00.00.00) |
| | | HUMAN GENE ONTOLOGY |
| | | BIOLOGICAL PROCESS |
| | | phagocytosis > phagocytosis, engulfment |
| | | defence response > immune response |
| | | MOLECULAR FUNCTION |
| | | GO molecular function > cell adhesion |
| | | defense/immunity protein > immunoglobulin |
| | | B cell receptor > immunoglobulin |
| | | CELL COMPONENT |
| | | cell > membrane fraction |
| | | cell > plasma membrane |
| | | HUMAN PROTEIN DOMAINS (INTERPRO |
| | | SIGNATURES) |
| | | IPR003598 (IGc2) |
| | | IPR003599 (IG) |
| | | IPR003006 (ig) |
| hP27-009.1 | SEQ ID NO: 28 | HUMAN PANTHER CLASSIFICATIONS |
| | | FAMILY (SUBFAMILY) |
| | | CF10153(POTASSIUM |
| | | INTERMEDIATE/SMALL CONDUCTANCE |
| | | CALCIUM-ACTIVATED CHANNEL) |
| | | BIOLOGICAL PROCESS |
| | | Transport(2.15.00.00.00) > Ion |
| | | transport(2.15.01.00.00) > Cation |
| | | transport(2.15.01.01.00) |
| | | Neuronal activities(2.18.00.00.00) > Synaptic |
| | | transmission(2.18.01.00.00) |
| | | MOLECULAR FUNCTIONS |
| | | Ion channel(1.03.00.00.00) > Voltage- |
| | | gated ion channel(1.03.03.00.00) > Voltage-gated |
| | | potassium channel(1.03.03.03.00) |
| | | HUMAN GENE ONTOLOGY |
| | | No Gene Ontology |
| | | HUMAN PROTEIN DOMAINS (INTERPRO |
| | | SIGNATURES) |
| | | IPR003931 (SK channel) |
| | | IPR000694 (PRO RICH) |
| | | NULL (GLN RICH) |
| hP27-009.2 | SEQ ID NO: 30 | HUMAN PANTHER CLASSIFICATIONS |
| | | FAMILY (SUBFAMILY) |
| | | CF10153(POTASSIUM |
| | | INTERMEDIATE/SMALL CONDUCTANCE |
| | | CALCIUM-ACTIVATED CHANNEL) |
| | | BIOLOGICAL PROCESS |
| | | Transport(2.15.00.00.00) > Ion |
| | | transport(2.15.01.00.00) > Cation |
| | | transport(2.15.01.01.00) |
| | | Neuronal activities(2.18.00.00.00) > Synaptic |
| | | transmission(2.18.01.00.00) |
| | | MOLECULAR FUNCTIONS |
| | | Ion channel(1.03.00.00.00) > Voltage- |
| | | gated ion channel(1.03.03.00.00) > Voltage-gated |
| | | potassium channel(1.03.03.03.00) |
| | | HUMAN GENE ONTOLOGY |
| | | No Gene Ontology |
| | | HUMAN PROTEIN DOMAINS (INTERPRO |
| | | SIGNATURES) |
| | | IPR003931 (SKCHANNEL) |
| | | IPR004178 (CaMBD) |
| | | IPR003931 (SK channel) |
| | | IPR000694 (PRO RICH) |
| | | NULL (GLN RICH) |
| | | IPR001622 (CHANNEL PORE K) |
| hP27-014.1 | SEQ ID NO: 42 | HUMAN PANTHER CLASSIFICATIONS |
| | | FAMILY (SUBFAMILY) |
| | | MACROPHAGE COLONY |
| | | STIMULATING FACTOR-1(MACROPHAGE |
| | | COLONY STIMULATING FACTOR-1) |
| | | BIOLOGICAL PROCESS |
| | | Immunity and defense(2.16.00.00.00) > Macrophage- |
| | | mediated immunity(2.16.05.00.00) |
| | | Immunity and defense(2.16.00.00.00) > Granulocyte- |
| | | mediated immunity(2.16.04.00.00) |
| | | Developmental processes(2.23.00.00.00) > Mesoderm |
| | | development(2.23.10.00.00) > Hematopoesis |
| | | (2.23.10.06.00) |
| | | MOLECULAR FUNCTIONS |
| | | Signaling molecule(1.02.00.00.00) > Cytokine |
| | | (1.02.01.00.00) > Other |
| | | cytokine(1.02.01.99.00) |
| | | HUMAN GENE ONTOLOGY |
| | | No Gene Ontology |
| | | HUMAN PROTEIN DOMAINS (INTERPRO |
| | | SIGNATURES) |
| | | No Domain Hit |
| hP27-014.2 | SEQ ID NO: 44 | HUMAN PANTHER CLASSIFICATIONS |
| | | FAMILY (SUBFAMILY) |
| | | MACROPHAGE COLONY |
| | | STIMULATING FACTOR-1(MACROPHAGE |
| | | COLONY STIMULATING FACTOR-1) |
| | | BIOLOGICAL PROCESS |
| | | Immunity and defense(2.16.00.00.00) > Macrophage- |
| | | mediated immunity(2.16.05.00.00) |
| | | Immunity and defense(2.16.00.00.00) > Granulocyte- |
| | | mediated immunity(2.16.04.00.00) |
| | | Developmental processes(2.23.00.00.00) > Mesoderm |
| | | development(2.23.10.00.00) > Hematopoesis |
| | | (2.23.10.06.00) |
| | | MOLECULAR FUNCTIONS |
| | | Signaling molecule(1.02.00.00.00) > Cytokine |
| | | (1.02.01.00.00) > Other |
| | | cytokine(1.02.01.99.00) |
| | | HUMAN GENE ONTOLOGY |
| | | No Gene Ontology |
| | | HUMAN PROTEIN DOMAINS (INTERPRO |
| | | SIGNATURES) |
| | | No Domain Hit |
| hP27-014.3 | SEQ ID NO: 46 | HUMAN PANTHER CLASSIFICATIONS |
| | | FAMILY (SUBFAMILY) |
| | | MACROPHAGE COLONY |
| | | STIMULATING FACTOR-1(Unassigned) |
| | | BIOLOGICAL PROCESS |
| | | Immunity and defense(2.16.00.00.00) > Macrophage- |
| | | mediated immunity(2.16.05.00.00) |
| | | Immunity and defense(2.16.00.00.00) > Granulocyte- |
| | | mediated immunity(2.16.04.00.00) |
| | | Developmental processes(2.23.00.00.00) > Mesoderm |
| | | development(2.23.10.00.00) > Hematopoesis |
| | | (2.23.10.06.00) |
| | | MOLECULAR FUNCTIONS |
| | | Signaling molecule(1.02.00.00.00) > Cytokine |
| | | (1.02.01.00.00) > Other |
| | | cytokine(1.02.01.99.00) |
| | | HUMAN GENE ONTOLOGY |
| | | No Gene Ontology |
| | | HUMAN PROTEIN DOMAINS (INTERPRO |
| | | SIGNATURES) |
| | | No Domain Hit |
| hP27-014.4 | SEQ ID NO: 48 | HUMAN PANTHER CLASSIFICATIONS |
| | | FAMILY (SUBFAMILY) |
| | | MACROPHAGE COLONY |
| | | STIMULATING FACTOR-1(MACROPHAGE |
| | | COLONY STIMULATING FACTOR-1) |
| | | BIOLOGICAL PROCESS |
| | | Immunity and defense(2.16.00.00.00) > Macrophage- |
| | | mediated immunity(2.16.05.00.00) |
| | | Immunity and defense(2.16.00.00.00) > Granulocyte- |
| | | mediated immunity(2.16.04.00.00) |
| | | Developmental processes(2.23.00.00.00) > Mesoderm |
| | | development(2.23.10.00.00) > Hematopoesis |
| | | (2.23.10.06.00) |
| | | MOLECULAR FUNCTIONS |
| | | Signaling molecule(1.02.00.00.00) > Cytokine |
| | | (1.02.01.00.00) > Other |
| | | cytokine(1.02.01.99.00) |
| | | HUMAN GENE ONTOLOGY |
| | | No Gene Ontology |
| | | HUMAN PROTEIN DOMAINS (INTERPRO |
| | | SIGNATURES) |
| | | No Domain Hit |
| hP28-002.1 | SEQ ID NO: 54 | HUMAN PANTHER CLASSIFICATIONS |
| | | FAMILY (SUBFAMILY) |
| | | TETRASPANIN- |
| | | RELATED(TETRASPANIN TSPAN-5) |
| | | BIOLOGICAL PROCESS |
| | | Cell adhesion(2.29.00.00.00) |
| | | MOLECULAR FUNCTIONS |
| | | Cell adhesion molecule(1.05.00.00.00) > Other |
| | | cell adhesion molecule(1.05.99.00.00) |
| | | HUMAN GENE ONTOLOGY |
| | | No Gene Ontology |
| | | HUMAN PROTEIN DOMAINS (INTERPRO |
| | | SIGNATURES) |
| | | IPR000301 (TMFOUR) |
| | | IPR000301 (transmembrane4) |
| | | IPR000301 (TM4 2) |
| hP28-010.1 | SEQ ID NO: 62 | HUMAN PANTHER CLASSIFICATIONS |
| | | FAMILY (SUBFAMILY) |
| | | CADHERIN(LIVER-INTESTINE- |
| | | CADHERIN) |
| | | BIOLOGICAL PROCESS |
| | | Signal transduction(2.11.00.00.00) > Cell |
| | | communication(2.11.03.00.00) > Cell adhesion- |
| | | mediated signaling(2.11.03.01.00) |
| | | Cell adhesion(2.29.00.00.00) |
| | | MOLECULAR FUNCTIONS |
| | | Cell adhesion molecule(1.05.00.00.00) > Cadherin |
| | | (1.05.02.00.00) |
| | | HUMAN GENE ONTOLOGY |
| | | BIOLOGICAL PROCESS |
| | | cell adhesion > homophilic cell adhesion |
| | | neurogenesis > central nervous system |
| | | development |
| | | transcription, DNA-dependent > transcription |
| | | regulation |
| | | cell death > apoptosis |
| | | peptidoglycan catabolism > microtubule- |
| | | based movement |
| | | microtubule-based process > microtubule- |
| | | based movement |
| | | nuclear congression > microtubule-based |
| | | movement |
| | | MOLECULAR FUNCTION |
| | | cell adhesion > calcium-dependent cell |
| | | adhesion |
| | | ligand binding or carrier > calcium binding |
| | | enzyme > nitric oxide synthase |
| | | GO molecular function > cell cycle |
| | | regulator |
| | | CELL COMPONENT |
| | | cell > membrane fraction |
| | | mitochondrial membrane > mitochondrial |
| | | inner membrane |
| | | GO cellular component > extracellular |
| | | extracellular > extracellular space |
| | | HUMAN PROTEIN DOMAINS (INTERPRO |
| | | SIGNATURES) |
| | | IPR002126 (CADHERIN) |
| | | IPR002126 (CA) |
| | | IPR002126 (cadherin) |
| | | IPR002126 (CADHERIN 2 6) |
| hP28-021.1 | SEQ ID NO: 68 | HUMAN PANTHER CLASSIFICATIONS |
| | | FAMILY (SUBFAMILY) |
| | | CELL ADHESION MOLECULE- |
| | | RELATED(RECEPTOR PROTEIN TYROSINE |
| | | PHOSPHATASE) |
| | | BIOLOGICAL PROCESS |
| | | Protein metabolism and |
| | | modification(2.05.00.00.00) > Protein |
| | | modification(2.05.03.00.00) > Protein |
| | | phosphorylation(2.05.03.01.00) |
| | | Signal transduction(2.11.00.00.00) > Cell |
| | | surface receptor mediated signal |
| | | transduction(2.11.01.00.00) > Other receptor |
| | | mediated signaling pathway(2.11.01.99.00) |
| | | MOLECULAR FUNCTIONS |
| | | Receptor(1.01.00.00.00) > Other |
| | | receptor(1.01.99.00.00) |
| | | Phosphatase(1.16.00.00.00) > Protein |
| | | phosphatase(1.16.01.00.00) |
| | | HUMAN GENE ONTOLOGY |
| | | BIOLOGICAL PROCESS |
| | | protein modification > protein |
| | | dephosphorylation |
| | | enzyme linked receptor protein signaling |
| | | pathway > transmembrane receptor protein |
| | | tyrosine phosphatase signaling pathway |
| | | isoprenoid catabolism > phosphate |
| | | metabolism |
| | | defasciculation of neuron > defasciculation |
| | | of motor neuron |
| | | enzyme linked receptor protein signaling |
| | | pathway > transmembrane receptor protein |
| | | tyrosine kinase signaling pathway |
| | | MOLECULAR FUNCTION |
| | | enzyme > protein phosphatase |
| | | protein phosphatase > protein tyrosine |
| | | phosphatase |
| | | protein tyrosine phosphatase > prenylated |
| | | protein tyrosine phosphatase |
| | | protein tyrosine phosphatase > transmembrane |
| | | receptor protein tyrosine |
| | | phosphatase |
| | | transmembrane receptor > transmembrane |
| | | receptor protein tyrosine phosphatase |
| | | protein tyrosine phosphatase > non- |
| | | membrane spanning protein tyrosine phosphatase |
| | | CELL COMPONENT |
| | | cell > membrane fraction |
| | | cytoplasm > cytoskeleton |
| | | cell > plasma membrane |
| | | plasma membrane > integral plasma |
| | | membrane protein |
| | | cell > soluble fraction |
| | | HUMAN PROTEIN DOMAINS (INTERPRO |
| | | SIGNATURES) |
| | | IPR003962 (FNTYPEIII) |
| | | IPR000242 (PRTYPHPHTASE) |
| | | IPR000242 (PTPc) |
| | | IPR003961 (FN3) |
| | | IPR003598 (IGc2) |
| | | IPR003599 (IG) |
| | | IPR003595 (PTPc motif) |
| | | IPR003961 (fn3) |
| | | IPR000242 (Y phosphatase) |
| | | IPR003006 (ig) |
| | | IPR000387 (TYR PHOSPHATASE 2 2) |
| | | IPR000242 (TYR PHOSPHATASE PTP2) |
| hP28-021.2 | SEQ ID NO: 70 | HUMAN PANTHER CLASSIFICATIONS |
| | | FAMILY (SUBFAMILY) |
| | | CELL ADHESION MOLECULE- |
| | | RELATED(RECEPTOR PROTEIN TYROSINE |
| | | PHOSPHATASE) |
| | | BIOLOGICAL PROCESS |
| | | Protein metabolism and |
| | | modification(2.05.00.00.00) > Protein |
| | | modification(2.05.03.00.00) > Protein |
| | | phosphorylation(2.05.03.01.00) |
| | | Signal transduction(2.11.00.00.00) > Cell |
| | | surface receptor mediated signal |
| | | transduction(2.11.01.00.00) > Other receptor |
| | | mediated signaling pathway(2.11.01.99.00) |
| | | MOLECULAR FUNCTIONS |
| | | Receptor(1.01.00.00.00) > Other |
| | | receptor(1.01.99.00.00) |
| | | Phosphatase(1.16.00.00.00) > Protein |
| | | phosphatase(1.16.01.00.00) |
| | | HUMAN GENE ONTOLOGY |
| | | BIOLOGICAL PROCESS |
| | | protein modification > protein |
| | | dephosphorylation |
| | | enzyme linked receptor protein signaling |
| | | pathway > transmembrane receptor protein |
| | | tyrosine phosphatase signaling pathway |
| | | isoprenoid catabolism > phosphate |
| | | metabolism |
| | | defasciculation of neuron > defasciculation |
| | | of motor neuron |
| | | enzyme linked receptor protein signaling |
| | | pathway > transmembrane receptor protein |
| | | tyrosine kinase signaling pathway |
| | | MOLECULAR FUNCTION |
| | | enzyme > protein phosphatase |
| | | protein phosphatase > protein tyrosine |
| | | phosphatase |
| | | protein tyrosine phosphatase > prenylated |
| | | protein tyrosine phosphatase |
| | | protein tyrosine phosphatase > transmembrane |
| | | receptor protein tyrosine |
| | | phosphatase |
| | | transmembrane receptor > transmembrane |
| | | receptor protein tyrosine phosphatase |
| | | protein tyrosine phosphatase > non- |
| | | membrane spanning protein tyrosine phosphatase |
| | | CELL COMPONENT |
| | | cell > membrane fraction |
| | | cytoplasm > cytoskeleton |
| | | cell > plasma membrane |
| | | plasma membrane > integral plasma |
| | | membrane protein |
| | | cell > soluble fraction |
| | | HUMAN PROTEIN DOMAINS (INTERPRO |
| | | SIGNATURES) |
| | | IPR003962 (FNTYPEIII) |
| | | IPR000242 (PRTYPHPHTASE) |
| | | IPR000242 (PTPc) |
| | | IPR003961 (FN3) |
| | | IPR003598 (IGc2) |
| | | IPR003599 (IG) |
| | | IPR003595 (PTPc motif) |
| | | IPR003961 (fn3) |
| | | IPR000242 (Y phosphatase) |
| | | IPR003006 (ig) |
| | | IPR000387 (TYR PHOSPHATASE 2 2) |
| | | IPR000242 (TYR PHOSPHATASE PTP2) |
| hP28-021.3 | SEQ ID NO: 72 | HUMAN PANTHER CLASSIFICATIONS |
| | | FAMILY (SUBFAMILY) |
| | | CELL ADHESION MOLECULE- |
| | | RELATED(RECEPTOR PROTEIN TYROSINE |
| | | PHOSPHATASE) |
| | | BIOLOGICAL PROCESS |
| | | Protein metabolism and |
| | | modification(2.05.00.00.00) > Protein |
| | | modification(2.05.03.00.00) > Protein |
| | | phosphorylation(2.05.03.01.00) |
| | | Signal transduction(2.11.00.00.00) > Cell |
| | | surface receptor mediated signal |
| | | transduction(2.11.01.00.00) > Other receptor |
| | | mediated signaling pathway(2.11.01.99.00) |
| | | MOLECULAR FUNCTIONS |
| | | Receptor(1.01.00.00.00) > Other |
| | | receptor(1.01.99.00.00) |
| | | Phosphatase(1.16.00.00.00) > Protein |
| | | phosphatase(1.16.01.00.00) |
| | | HUMAN GENE ONTOLOGY |
| | | BIOLOGICAL PROCESS |
| | | protein modification > protein |
| | | dephosphorylation |
| | | enzyme linked receptor protein signaling |
| | | pathway > transmembrane receptor protein |
| | | tyrosine phosphatase signaling pathway |
| | | isoprenoid catabolism > phosphate |
| | | metabolism |
| | | defasciculation of neuron > defasciculation |
| | | of motor neuron |
| | | enzyme linked receptor protein signaling |
| | | pathway > transmembrane receptor protein |
| | | tyrosine kinase signaling pathway |
| | | MOLECULAR FUNCTION |
| | | enzyme > protein phosphatase |
| | | protein phosphatase > protein tyrosine |
| | | phosphatase |
| | | protein tyrosine phosphatase > prenylated |
| | | protein tyrosine phosphatase |
| | | protein tyrosine phosphatase > transmembrane |
| | | receptor protein tyrosine |
| | | phosphatase |
| | | transmembrane receptor > transmembrane |
| | | receptor protein tyrosine phosphatase |
| | | protein tyrosine phosphatase > non- |
| | | membrane spanning protein tyrosine phosphatase |
| | | CELL COMPONENT |
| | | cell > membrane fraction |
| | | cytoplasm > cytoskeleton |
| | | cell > plasma membrane |
| | | plasma membrane > integral plasma |
| | | membrane protein |
| | | cell > soluble fraction |
| | | HUMAN PROTEIN DOMAINS (INTERPRO |
| | | SIGNATURES) |
| | | IPR003962 (FNTYPEIII) |
| | | IPR000242 (PRTYPHPHTASE) |
| | | IPR000242 (PTPc) |
| | | IPR003961 (FN3) |
| | | IPR003598 (IGc2) |
| | | IPR003599 (IG) |
| | | IPR003595 (PTPc motif) |
| | | IPR003961 (fn3) |
| | | IPR000242 (Y phosphatase) |
| | | IPR003006 (ig) |
| | | IPR000387 (TYR PHOSPHATASE 2 2) |
| | | IPR000242 (TYR PHOSPHATASE PTP2) |
| hP28-021.4 | SEQ ID NO: 74 | HUMAN PANTHER CLASSIFICATIONS |
| | | FAMILY (SUBFAMILY) |
| | | CELL ADHESION MOLECULE- |
| | | RELATED(RECEPTOR PROTEIN TYROSINE |
| | | PHOSPHATASE) |
| | | BIOLOGICAL PROCESS |
| | | Protein metabolism and |
| | | modification(2.05.00.00.00) > Protein |
| | | modification(2.05.03.00.00) > Protein |
| | | phosphorylation(2.05.03.01.00) |
| | | Signal transduction(2.11.00.00.00) > Cell |
| | | surface receptor mediated signal |
| | | transduction(2.11.01.00.00) > Other receptor |
| | | mediated signaling pathway(2.11.01.99.00) |
| | | MOLECULAR FUNCTIONS |
| | | Receptor(1.01.00.00.00) > Other |
| | | receptor(1.01.99.00.00) |
| | | Phosphatase(1.16.00.00.00) > Protein |
| | | phosphatase(1.16.01.00.00) |
| | | HUMAN GENE ONTOLOGY |
| | | BIOLOGICAL PROCESS |
| | | protein modification > protein |
| | | dephosphorylation |
| | | enzyme linked receptor protein signaling |
| | | pathway > transmembrane receptor protein |
| | | tyrosine phosphatase signaling pathway |
| | | isoprenoid catabolism > phosphate |
| | | metabolism |
| | | defasciculation of neuron > defasciculation |
| | | of motor neuron |
| | | enzyme linked receptor protein signaling |
| | | pathway > transmembrane receptor protein |
| | | tyrosine kinase signaling pathway |
| | | MOLECULAR FUNCTION |
| | | enzyme > protein phosphatase |
| | | protein phosphatase > protein tyrosine |
| | | phosphatase |
| | | protein tyrosine phosphatase > prenylated |
| | | protein tyrosine phosphatase |
| | | protein tyrosine phosphatase > transmembrane |
| | | receptor protein tyrosine |
| | | phosphatase |
| | | transmembrane receptor > transmembrane |
| | | receptor protein tyrosine phosphatase |
| | | protein tyrosine phosphatase > non- |
| | | membrane spanning protein tyrosine phosphatase |
| | | CELL COMPONENT |
| | | cell > membrane fraction |
| | | cytoplasm > cytoskeleton |
| | | cell > plasma membrane |
| | | plasma membrane > integral plasma |
| | | membrane protein |
| | | cell > soluble fraction |
| | | HUMAN PROTEIN DOMAINS (INTERPRO |
| | | SIGNATURES) |
| | | IPR003962 (FNTYPEIII) |
| | | IPR000242 (PRTYPHPHTASE) |
| | | IPR000242 (PTPc) |
| | | IPR003961 (FN3) |
| | | IPR003598 (IGc2) |
| | | IPR003599 (IG) |
| | | IPR003595 (PTPc motif) |
| | | IPR003961 (fn3) |
| | | IPR000242 (Y phosphatase) |
| | | IPR003006 (ig) |
| | | IPR000387 (TYR PHOSPHATASE 2 2) |
| | | IPR000242 (TYR PHOSPHATASE PTP2) |

A CA protein (CAP) is expressed on a cell surface, wherein the CA protein is selected from the group consisting of SEQ ID NOS: 8, 10, 18, 20, 22, 28, 30, 42, 44, 46, 48, 54, 62, 68, 70, 72 and 74 as shown in Tables 1-7.

Certain aspects of the present invention are described in greater detail in the non-limiting examples that follow.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Insertion Site Analysis Following Tumor Induction in Mice

Tumors are induced in mice using either mouse mammary tumor virus (MMTV) or murine leukemia virus (MLV). MMTV causes mammary adenocarcinomas and MLV causes a variety of different hematopoetic malignancies (primarily T- or B-cell lymphomas). Three routes of infection are used: (1) injection of neonates with purified virus preparations, (2) infection by milk-borne virus during nursing, and (3) genetic transmission of pathogenic proviruses via the germ-line (Akvr1 and/or Mtv2). The type of malignancy present in each affected mouse is determined by histological analysis of H&E-stained thin sections of formalin-fixed, paraffin-embedded biopsy samples. Host DNA sequences flanking all clonally-integrated proviruses in each tumor are recovered by nested anchored-PCR using two virus-specific primers and two primers specific for a 40 bp double stranded DNA anchor ligated to restriction enzyme digested tumor DNA. Amplified bands representing host/virus junction fragments are cloned and sequenced. Then the host sequences (called “tags”) are used to BLAST analyze the Celera mouse genomic sequence. For each individual tag, three parameters are recorded: (1) the mouse chromosome assignment, (2) base pair coordinates at which the integration occurred, and (3) provirus orientation. Using this information, all available tags from all analyzed tumors are mapped to the mouse genome. To identify the protooncogene targets of provirus insertion mutation, the provirus integration pattern at each cluster of integrants is analyzed relative to the locations of all known genes in the transcriptome. The presence of provirus at the same locus in two or more independent tumors is prima facie evidence that a protooncogene is present at or very near the proviral integration sites. This is because the genome is too large for random integrations to result in observable clustering. Any clustering that is detected is unequivocal evidence for biological selection during tumorigenesis. In order to identify the human orthologs of the protooncogene targets of provirus insertion mutation, a comparative analysis of syntenic regions of the mouse and human genomes is performed.

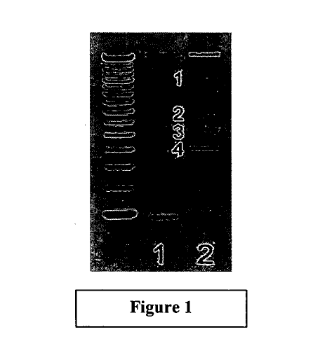

An example of PCR amplification of host/virus junction fragments is presented in FIG. 1. Lane 1 contains the amplification products from normal control DNA and lane 2 contains the amplification products from tumor DNA. The bands result from 5′ host/virus junction fragments present in the DNA samples. Lane 1 has bands from the env/3′ LTR junctions from all proviruses (upper) and the host/5′ LTR from the pathogenic endogenous Mtv2 provirus present in this particular mouse strain. This endogenous provirus is detected because its sequence is identical to the new clonally integrated proviruses in the tumor. All four new clonally integrated proviruses known to be in this tumor are readily detected.

### Example 2

### Analysis of Quantitative RT-PCR: Comparative C_{T} Method.

The expression level of target genes is quantified using the ABI PRISM 7900HT Sequence Detection System (Applied Biosystems, California). The method is based on the quantitation of the initial copy number of target template in comparison to that of a reference (normalizer) housekeeper gene (Pre-Developed TaqMang Assay Reagents Gene Expression Quantification Protocol, Applied Biosystems, 2001). Accumulation of DNA product with each PCR cycle is related to amplicon efficiency and the initial template concentration. Therefore the amplification efficiency of both the target and the normalizer must be approximately equal. The threshold cycle (CT), which is dependent on the starting template copy number and the DNA amplification efficiency, is a PCR cycle during which PCR product growth is exponential. With a similar dynamic range for the target and normalizer, the comparative CT method is applicable.

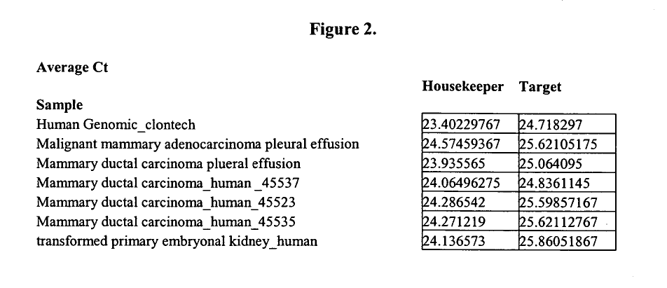

An example of the comparative CT method of gene expression for quantitative RT-PCR is shown in FIG. 2. In the first step, assays are performed in quadruplicate on a normal tissue and several sample tissues. In these tissues, the means and standard deviations of CT values are determined for housekeeper genes (chosen as controls if shown to be biologically stable among various samples, irrespective of disease state) and for the target gene. FIG. 2 shows an example of average CT values for a housekeeper gene and target gene. These values can fall within a range from upper teens to 40 depending on the intrinsic expression level of the gene in the particular tissue. The coefficient of variance of all replicate sets cannot exceed 1.5%.

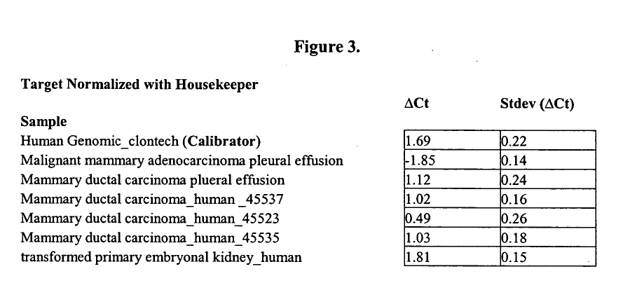

An assessment of how the ΔC_{T} changes with template dilution verifies that the efficiencies of the target and housekeeper amplicons are approximately equal if the log input amount of template RNA versus ΔC_{T} plot has a slope <0.10. With the relative efficiencies verified for target and housekeeper, the ΔΔC_{T} comparative calculation becomes valid, as mentioned above. An example of the calculated difference between the CT values of target and housekeeper genes (ΔC_{T}) for various samples is shown in FIG. 3. The ΔΔC_{T} is calculated for each sample by subtracting its ΔC_{T} value from the ΔC_{T} value of the baseline (calibrator) sample. If the expression is increased in some samples and decreased in others, ΔΔC_{T} will be a mixture of negative and positive values. The final step in the calculation is to transform these values to absolute values. The formula for this is:
Comparative expression level=2^{-ΔΔCT}

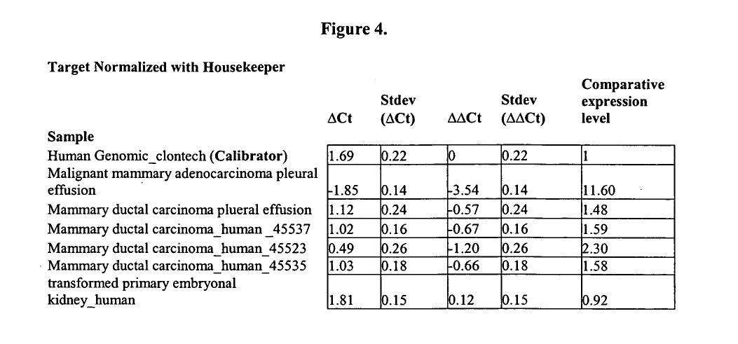

The final value for the calibrator should always be one. FIG. 4 shows the ΔΔC_{T} and comparative expression level for each sample from FIG. 3.

### Example 3

### Detection of Elevated Levels of cDNA Associated with Cancer Using Arrays

cDNA sequences representing a variety of candidate CA genes to be screened for differential expression in cancer are assayed by hybridization on polynucleotide arrays. The cDNA sequences include cDNA clones isolated from cell lines or tissues of interest. The cDNA sequences analyzed also include polynucleotides comprising sequence overlap with sequences in the Unigene database, and which encode a variety of gene products of various origins, functionality, and levels of characterization. cDNAs are spotted onto reflective slides (Amersham) according to methods well known in the art at a density of 9,216 spots per slide representing 4,068 sequences (including controls) spotted in duplicate, with approximately 0.8 μl of an approximately 200 ng/μl solution of cDNA.

PCR products of selected cDNA clones corresponding to the gene products of interest are prepared in a 50% DMSO solution. These PCR products are spotted onto Amersham aluminum microarray slides at a density of 9216 clones per array using a Molecular Dynamics Generation III spotting robot. Clones are spotted in duplicate, for a total of 4608 different sequences per chip.
cDNA probes are prepared from total RNA obtained by laser capture microdissection (LCM, Arcturus Enginering Inc., Mountain View, Calif.) of tumor tissue samples and normal tissue samples isolated from patients.

Total RNA is first reverse transcribed into cDNA using a primer containing a T7 RNA polymerase promoter, followed by second strand DNA synthesis. cDNA is then transcribed in vitro to produce antisense RNA using the T7 promoter-mediated expression (see, e.g., Luo et al. (1999) Nature Med 5:117-122), and the antisense RNA is then converted into cDNA. The second set of cDNAs are again transcribed in vitro, using the T7 promoter, to provide antisense RNA. This antisense RNA is then fluorescently labeled, or the RNA is again converted into cDNA, allowing for a third round of T7-mediated amplification to produce more antisense RNA. Thus the procedure provides for two or three rounds of in vitro transcription to produce the final RNA used for fluorescent labeling. Probes are labeled by making fluorescently labeled cDNA from the RNA starting material. Fluorescently labeled cDNAs prepared from the tumor RNA sample are compared to fluorescently labeled cDNAs prepared from normal cell RNA sample. For example, the cDNA probes from the normal cells are labeled with Cy3 fluorescent dye (green) and the cDNA probes prepared from suspected cancer cells are labeled with Cy5 fluorescent dye (red).

The differential expression assay is performed by mixing equal amounts of probes from tumor cells and normal cells of the same patient. The arrays are prehybridized by incubation for about 2 hrs at 60° C. in 5×SSC, 0.2% SDS, 1 mM EDTA, and then washing three times in water and twice in isopropanol. Following prehybridization of the array, the probe mixture is then hybridized to the array under conditions of high stringency (overnight at 42° C. in 50% formamide, 5×SSC, and 0.2% SDS. After hybridization, the array is washed at 55° C. three times as follows: 1) first wash in 1×SSC/0.2% SDS; 2) second wash in 0.1×SSC/0.2% SDS; and 3) third wash in 0.1×SSC.

The arrays are then scanned for green and red fluorescence using a Molecular Dynamics Generation III dual color laser-scanner/detector. The images are processed using BioDiscovery Autogene software, and the data from each scan set normalized. The experiment is repeated, this time labeling the two probes with the opposite color in order to perform the assay in both “color directions.” Each experiment is sometimes repeated with two more slides (one in each color direction). The data from each scan is normalized, and the level of fluorescence for each sequence on the array expressed as a ratio of the geometric mean of 8 replicate spots/genes from the four arrays or 4 replicate spots/gene from 2 arrays or some other permutation.

Normalization: The objective of normalization is to generate a cDNA library in which all transcripts expressed in a particular cell type or tissue are equally represented (S. M. Weissman, Mol. Biol. Med. 4(3):133-143 (1987); Patanjali, et al., Proc. Natl. Acad. Sci. USA 88(5):1943-1947 (1991)), and therefore isolation of as few as 30,000 recombinant clones in an optimally normalized library may represent the entire gene expression repertoire of a cell, estimated to number 10,000 per cell.

Total RNA is extracted from harvested cells using RNeasy™ Protect Kit (Qiagen, Valencia, Calif.), following manufacturer's recommended procedures. RNA is quantified using RiboGreen™ RNA quantification kit (Molecular Probes, Inc. Eugene, Oreg.). One μg of total RNA is reverse transcribed and PCR amplified using SMART™ PCR cDNA synthesis kit (ClonTech, Palo Alto, Calif.). The cDNA products are size-selected by agarose gel electrophoresis using standard procedures (Sambrook, J. T., et al. Molecular Cloning: A Laboratory Manual, 2d ed., Cold Spring Harbor Laboratory Press, NY). The cDNA is extracted using Bio 101 Geneclean® II kit (Qbiogene, Carlsbad, Calif.). Normalization of the cDNA is carried out using kinetics of hybridization principles: 1.0 μg of cDNA is denatured by heat at 100° C. for 10 minutes, then incubated at 42° C. for 42 hours in the presence of 120 mM NaCl, 10 mM Tris.HCl (pH=8.0), 5 mM EDTA.Na+ and 50% formamide. Single-stranded cDNA (“normalized”) is purified by hydroxyapatite chromatography (#130-0520, BioRad, Hercules, Calif.) following the manufacturer's recommended procedures, amplified and converted to double-stranded cDNA by three cycles of PCR amplification, and cloned into plasmid vectors using standard procedures (Sambrook, J. T., et al. Molecular Cloning: A Laboratory Manual, 2d ed., Cold Spring Harbor Laboratory Press, NY). All primers/adaptors used in the normalization and cloning process are provided by the manufacturer in the SMART™ PCR cDNA synthesis kit (ClonTech, Palo Alto, Calif.). Supercompetent cells (XL-2 Blue Ultracompetent Cells, Stratagene, California) are transfected with the normalized cDNA libraries, plated on solid media and grown overnight at 36° C.

The sequences of 10,000 recombinants per normalized library are analyzed by capillary sequencing using the ABI PRISM 3700 DNA Analyzer (Applied Biosystems, California). To determine the representation of transcripts in a library, BLAST analysis is performed on the clone sequences to assign transcript identity to each isolated clone, i.e., the sequences of the isolated polynucleotides are first masked to eliminate low complexity sequences using the XBLAST masking program (Clayerie “Effective Large-Scale Sequence Similarity Searches,” Computer Methods for Macromolecular Sequence Analysis, Doolittle, ed., Meth. Enzymol. 266:212-227 Academic Press, NY, N.Y. (1996); see particularly Clayerie, in “Automated DNA Sequencing and Analysis Techniques” Adams et al., eds., Chap. 36, p. 267 Academic Press, San Diego, 1994 and Clayerie et al. Comput. Chem. (1993) 17:191). Generally, masking does not influence the final search results, except to eliminate sequences of relative little interest due to their low complexity, and to eliminate multiple “hits” based on similarity to repetitive regions common to multiple sequences, e.g., Alu repeats. The remaining sequences are then used in a BLASTN vs. GenBank search. The sequences are also used as query sequence in a BLASTX vs. NRP (non-redundant proteins) database search.

Automated sequencing reactions are performed using a Perkin-Elmer PRISM Dye Terminator Cycle Sequencing Ready Reaction Kit containing AmpliTaq DNA Polymerase, FS, according to the manufacturer's directions. The reactions are cycled on a GeneAmp PCR System 9600 as per manufacturer's instructions, except that they are annealed at 20° C. or 30° C. for one minute. Sequencing reactions are ethanol precipitated, pellets are resuspended in 8 microliters of loading buffer, 1.5 microliters is loaded on a sequencing gel, and the data is collected by an ABI PRISM 3700 DNA Sequencer. (Applied Biosystems, Foster City, Calif.).

The number of times a sequence is represented in a library is determined by performing sequence identity analysis on the cloned cDNA sequences and assigning transcript identity to each isolated clone. First, each sequence is checked to determine if it is a bacterial, ribosomal, or mitochondrial contaminant. Such sequences are excluded from the subsequent analysis. Second, sequence artifacts, such as vector and repetitive elements, are masked and/or removed from each sequence.

The remaining sequences are compared via BLAST (Altschul et. al, J. Mol. Biol., 215:40, 1990) to GenBank and EST databases for gene identification and are compared with each other via FastA (Pearson & Lipman, PNAS, 85:2444, 1988) to calculate the frequency of cDNA appearance in the normalized cDNA library. The sequences are also searched against the GenBank and GeneSeq nucleotide databases using the BLASTN program (BLASTN 1.3 MP: Altschul et al., J. Mol. Bio. 215:403, 1990). Fourth, the sequences are analyzed against a non-redundant protein (NRP) database with the BLASTX program (BLASTX 1.3 MP: Altschul et al., supra). This protein database is a combination of the Swiss-Prot, PIR, and NCBI GenPept protein databases. The BLASTX program is run using the default BLOSUM-62 substitution matrix with the filter parameter: “xnu+seg”. The score cutoff utilized is 75. Assembly of overlapping clones into contigs is done using the program Sequencher (Gene Codes Corp.; Ann Arbor, Mich.). The assembled contigs are analyzed using the programs in the GCG package (Genetic Computer Group, University Research Park, 575 Science Drive, Madison, Wis. 53711) Suite Version 10.1.

### Example 4

### Detection of CA-Sequences in Human Cancer Cells and Tissues

DNA from prostate and breast cancer tissues and other human cancer tissues, human colon, normal human tissues including non-cancerous prostate, and from other human cell lines are extracted following the procedure of Delli Bovi et al. (1986, Cancer Res. 46:6333-6338). The DNA is resuspended in a solution containing 0.05 M Tris HCl buffer, pH 7.8, and 0.1 mM EDTA, and the amount of DNA recovered is determined by microfluorometry using Hoechst 33258 dye. Cesarone, C. et al., Anal Biochem 100:188-197 (1979).

Polymerase chain reaction (PCR) is performed using Taq polymerase following the conditions recommended by the manufacturer (Perkin Elmer Cetus) with regard to buffer, Mg²⁺, and nucleotide concentrations. Thermocycling is performed in a DNA cycler by denaturation at 94° C. for 3 min. followed by either 35 or 50 cycles of 94° C. for 1.5 min., 50° C. for 2 min. and 72° C. for 3 min. The ability of the PCR to amplify the selected regions of the CA gene is tested by using a cloned CA polynucleotide(s) as a positive template(s). Optimal Mg²⁺, primer concentrations and requirements for the different cycling temperatures are determined with these templates. The master mix recommended by the manufacturer is used. To detect possible contamination of the master mix components, reactions without template are routinely tested.

Southern blotting and hybridization are performed as described by Southern, E. M., (J. Mol. Biol. 98:503-517, 1975), using the cloned sequences labeled by the random primer procedure (Feinberg, A. P., et al., 1983, Anal. Biochem. 132:6-13). Prehybridization and hybridization are performed in a solution containing 6×SSPE, 5% Denhardt's, 0.5% SDS, 50% formamide, 100 μg/ml denaturated salmon testis DNA, incubated for 18 hrs at 42° C., followed by washings with 2×SSC and 0.5% SDS at room temperature and at 37° C. and finally in 0.1×SSC with 0.5% SDS at 68° C. for 30 min (Sambrook et al., 1989, in “Molecular Cloning: A Laboratory Manual”, Cold Spring Harbor Lab. Press). For paraffin-embedded tissue sections the conditions described by Wright and Manos (1990, in “PCR Protocols”, Innis et al., eds., Academic Press, pp. 153-158) are followed using primers designed to detect a 250 bp sequence.

### Example 5

### Expression of Cloned Polynucleotides in Host Cells

To study the protein products of CA genes, restriction fragments from CA DNA are cloned into the expression vector pMT2 (Sambrook, et al., Molecular Cloning: A Laboratory Manual, Cold Spring Harbor Laboratory Press pp 16.17-16.22 (1989)) and transfected into COS cells grown in DMEM supplemented with 10% FCS. Transfections are performed employing calcium phosphate techniques (Sambrook, et al (1989) pp. 16.32-16.40, supra) and cell lysates are prepared forty-eight hours after transfection from both transfected and untransfected COS cells. Lysates are subjected to analysis by immunoblotting using anti-peptide antibody.

In immunoblotting experiments, preparation of cell lysates and electrophoresis are performed according to standard procedures. Protein concentration is determined using BioRad protein assay solutions. After semi-dry electrophoretic transfer to nitrocellulose, the membranes are blocked in 500 mM NaCl, 20 mM Tris, pH 7.5, 0.05% Tween-20 (TTBS) with 5% dry milk. After washing in TTBS and incubation with secondary antibodies (Amersham), enhanced chemiluminescence (ECL) protocols (Amersham) are performed as described by the manufacturer to facilitate detection.

### Example 6

### Generation of Antibodies Against Polypeptides

Polypeptides, unique to CA genes are synthesized or isolated from bacterial or other (e.g., yeast, baculovirus) expression systems and conjugated to rabbit serum albumin (RSA) with m-maleimido benzoic acid N-hydroxysuccinimide ester (NBMS) (Pierce, Rockford, Ill.). Immunization protocols with these peptides are performed according to standard methods. Initially, a pre-bleed of the rabbits is performed prior to immunization. The first immunization includes Freund's complete adjuvant and 500 μg conjugated peptide or 100 μg purified peptide. All subsequent immunizations, performed four weeks after the previous injection, include Freund's incomplete adjuvant with the same amount of protein. Bleeds are conducted seven to ten days after the immunizations.

For affinity purification of the antibodies, the corresponding CA polypeptide is conjugated to RSA with NBS, and coupled to CNBr-activated Sepharose (Pharmacia, Uppsala, Sweden). Antiserum is diluted 10-fold in 10 mM Tris-HCl, pH 7.5, and incubated overnight with the affinity matrix. After washing, bound antibodies are eluted from the resin with 100 mM glycine, pH 2.5.

### Example 7

### Generation of Monoclonal Antibodies Against a CA Polypeptide

A non-denaturing adjuvant (Ribi, R730, Corixa, Hamilton Mont.) is rehydrated to 4 ml in phosphate buffered saline. 100 μl of this rehydrated adjuvant is then diluted with 4001 of Hank's Balanced Salt Solution and this is then gently mixed with the cell pellet used for immunization. Approximately 500 μg conjugated peptide or 100 μg purified peptide and Freund's complete are injected into Balb/c mice via foot-pad, once a week. After 6 weeks of weekly injection, a drop of blood is drawn from the tail of each immunized animal to test the titer of antibodies against CA polypeptides using FACS analysis. When the titer reaches at least 1:2000, the mice are sacrificed in a CO₂ chamber followed by cervical dislocation. Lymph nodes are harvested for hybridoma preparation. Lymphocytes from mice with the highest titer are fused with the mouse myeloma line X63-Ag8.653 using 35% polyethylene glycol 4000. On day 10 following the fulsion, the hybridoma supernatants are screened for the presence of CAP-specific monoclonal antibodies by fluorescence activated cell sorting (FACS). Conditioned medium from each hybridoma is incubated for 30 minutes with a combined aliquot of PC3, Colo-205, LnCap, or Panc-1 cells. After incubation, the cell samples are washed, resuspended in 0.1 ml diluent and incubated with 1 μg/ml of FITC conjugated F(ab′)2 fragment of goat anti-mouse IgG for 30 min at 4° C. The cells are washed, resuspended in 0.5 ml FACS diluent and analyzed using a FACScan cell analyzer (Becton Dickinson; San Jose, Calif.). Hybridoma clones are selected for further expansion, cloning, and characterization based on their binding to the surface of one or more of cell lines which express the CA polypeptide as assessed by FACS. A hybridoma making a monoclonal antibody designated mAbCA which binds an antigen designated Ag-CA.x and an epitope on that antigen designated Ag-CA.x. 1 is selected.

### Example 8

### ELISA Assay for Detecting CA Related Antigens

To test blood samples for antibodies that bind specifically to recombinantly produced CA antigens, the following procedure is employed. After a recombinant CA related protein is purified, the recombinant protein is diluted in PBS to a concentration of 5 μg/ml (500 ng/100 μl). 100 microliters of the diluted antigen solution is added to each well of a 96-well Immulon 1 plate (Dynatech Laboratories, Chantilly, Va.), and the plate is then incubated for 1 hour at room temperature, or overnight at 4° C., and washed 3 times with 0.05% Tween 20 in PBS. Blocking to reduce nonspecific binding of antibodies is accomplished by adding to each well 200 μl of a 1% solution of bovine serum albumin in PBS/Tween 20 and incubation for 1 hour. After aspiration of the blocking solution, 100 μl of the primary antibody solution (anticoagulated whole blood, plasma, or serum), diluted in the range of 1/16 to 1/2048 in blocking solution, is added and incubated for 1 hour at room temperature or overnight at 4° C. The wells are then washed 3 times, and 100 μl of goat anti-human IgG antibody conjugated to horseradish peroxidase (Organon Teknika, Durham, N.C.), diluted 1/500 or 1/1000 in PBS/Tween 20, 100 μl of o-phenylenediamine dihydrochloride (OPD, Sigma) solution is added to each well and incubated for 5-15 minutes. The OPD solution is prepared by dissolving a 5 mg OPD tablet in 50 ml 1% methanol in H₂O and adding 50 μl 30% H₂O₂ immediately before use. The reaction is stopped by adding 25 l of 4M H₂SO₄. Absorbances are read at 490 nm in a microplate reader (Bio-Rad).

### Example 9

### Identification and Characterization of CA Antigen on Cancer Cell Surface

A cell pellet of proximately 25 μl packed cell volume of a cancer cell preparation is lysed by first diluting the cells to 0.5 ml in water followed by freezing and thawing three times. The solution is centrifuged at 14,000 rpm. The resulting pellet, containing the cell membrane fragments, is resuspended in 50 μl of SDS sample buffer (Invitrogen, Carlsbad, Calif.). The sample is heated at 80° C. for 5 minutes and then centrifuged for 2 minutes at 14,000 rpm to remove any insoluble materials.

The samples are analyzed by Western blot using a 4 to 20% polyacrylamide gradient gel in Tris-Glycine SDS (Invitrogen; Carlsbad Calif.) following the manufacturer's directions. Ten microliters of membrane sample are applied to one lane on the polyacrylamide gel. A separate 10 μL sample is reduced first by the addition of 2 μL of dithiothreitol (100 mM) with heating at 80° C. for 2 minutes and then loaded into another lane. Pre-stained molecular weight markers SeeBlue Plus2 (Invitrogen; Carlsbad, Calif.) are used to assess molecular weight on the gel. The gel proteins are transferred to a nitrocellulose membrane using a transfer buffer of 14.4 g/l glycine, 3 g/l of Tris Base, 10% methanol, and 0.05% SDS. The membranes are blocked, probed with a CAP-specific monoclonal antibody (at a concentration of 0.5 μg/ml), and developed using the Invitrogen WesternBreeze Chromogenic Kit-AntiMouse according to the manufacturer's directions. In the reduced sample of the tumor cell membrane samples, a prominent band is observed migrating at a molecular weight within about 10% of the predicted molecular weight of the corresponding CA protein.

### Example 10

### Preparation of Vaccines

The present invention also relates to a method of stimulating an immune response against cells that express CA polypeptides in a patient using CA polypeptides of the invention that act as an antigen produced by or associated with a malignant cell. This aspect of the invention provides a method of stimulating an immune response in a human against cancer cells or cells that express CA polynucleotides and polypeptides. The method comprises the step of administering to a human an immunogenic amount of a polypeptide comprising: (a) the amino acid sequence of a huma CA protein or (b) a mutein or variant of a polypeptide comprising the amino acid sequence of a human endogenous retrovirus CA protein.

### Example 11

### Generation of Transgenic Animals Expressing Polypeptides as a Means for Testing Therapeutics

CA nucleic acids are used to generate genetically modified non-human animals, or site specific gene modifications thereof, in cell lines, for the study of function or regulation of prostate tumor-related genes, or to create animal models of diseases, including prostate cancer. The term “transgenic” is intended to encompass genetically modified animals having an exogenous CA gene(s) that is stably transmitted in the host cells where the gene(s) may be altered in sequence to produce a modified protein, or having an exogenous CA LTR promoter operably linked to a reporter gene. Transgenic animals may be made through a nucleic acid construct randomly integrated into the genome. Vectors for stable integration include plasmids, retroviruses and other animal viruses, YACs, and the like. Of interest are transgenic mammals, e.g. cows, pigs, goats, horses, etc., and particularly rodents, e.g. rats, mice, etc.

The modified cells or animals are useful in the study of CA gene function and regulation. For example, a series of small deletions and/or substitutions may be made in the CA genes to determine the role of different genes in tumorigenesis. Specific constructs of interest include, but are not limited to, antisense constructs to block CA gene expression, expression of dominant negative CA gene mutations, and over-expression of a CA gene. Expression of a CA gene or variants thereof in cells or tissues where it is not normally expressed or at abnormal times of development is provided. In addition, by providing expression of proteins derived from CA in cells in which it is otherwise not normally produced, changes in cellular behavior can be induced.

DNA constructs for random integration need not include regions of homology to mediate recombination. Conveniently, markers for positive and negative selection are included. For various techniques for transfecting mammalian cells, see Keown et al., Methods in Enzymology 185:527-537 (1990).

For embryonic stem (ES) cells, an ES cell line is employed, or embryonic cells are obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells are transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting chimeric animals screened for cells bearing the construct. By providing for a different phenotype of the blastocyst and the ES cells, chimeric progeny can be readily detected.

The chimeric animals are screened for the presence of the modified gene and males and females having the modification are mated to produce homozygous progeny. If the gene alterations cause lethality at some point in development, tissues or organs are maintained as allogeneic or congenic grafts or transplants, or in in vitro culture. The transgenic animals may be any non-human mammal, such as laboratory animals, domestic animals, etc. The transgenic animals are used in functional studies, drug screening, etc., e.g. to determine the effect of a candidate drug on prostate cancer, to test potential therapeutics or treatment regimens, etc.

### Example 12

### Diagnostic Imaging Using CA Specific Antibodies

The present invention encompasses the use of antibodies to CA polypeptides to accurately stage cancer patients at initial presentation and for early detection of metastatic spread of cancer. Radioimmunoscintigraphy using monoclonal antibodies specific for CA polypeptides can provide an additional cancer-specific diagnostic test. The monoclonal antibodies of the instant invention are used for histopathological diagnosis of carcinomas.

Subcutaneous human xenografts of cancer cells in nude mice is used to test whether a technetium-99m (^{99m}Tc)-labeled monoclonal antibody of the invention can successfully image the xenografted cancer by external gamma scintography as described for seminoma cells by Marks, et al., Brit. J. Urol. 75:225 (1995). Each monoclonal antibody specific for a CA polypeptide is purified from ascitic fluid of BALB/c mice bearing hybridoma tumors by affinity chromatography on protein A-Sepharose. Purified antibodies, including control monoclonal antibodies such as an avidin-specific monoclonal antibody (Skea, et al., J. Immunol. 151:3557 (1993)) are labeled with ^{99m}Tc following reduction, using the methods of Mather, et al., J. Nucl. Med. 31:692 (1990) and Zhang et al., Nucl. Med. Biol. 19:607 (1992). Nude mice bearing human cancer cells are injected intraperitoneally with 200-500 μCi of ^{99m}Tc-labeled antibody. Twenty-four hours after injection, images of the mice are obtained using a Siemens ZLC3700 gamma camera equipped with a 6 mm pinhole collimator set approximately 8 cm from the animal. To determine monoclonal antibody biodistribution following imaging, the normal organs and tumors are removed, weighed, and the radioactivity of the tissues and a sample of the injectate are measured. Additionally, CA-specific antibodies conjugated to antitumor compounds are used for cancer-specific chemotherapy.

### Example 13

### Immunohistochemical Methods

Frozen tissue samples from cancer patients are embedded in an optimum cutting temperature (OCT) compound and quick-frozen in isopentane with dry ice. Cryosections are cut with a Leica 3050 CM mictrotome at thickness of 5 μm and thaw-mounted on vectabound-coated slides. The sections are fixed with ethanol at −20° C. and allowed to air dry overnight at room temperature. The fixed sections are stored at −80° C. until use. For immunohistochemistry, the tissue sections are retrieved and first incubated in blocking buffer (PBS, 5% normal goat serum, 0.1% Tween 20) for 30 minutes at room temperature, and then incubated with the CA protein-specific monoclonal antibody and control monoclonal antibodies diluted in blocking buffer (1 μg/ml) for 120 minutes. The sections are then washed three times with the blocking buffer. The bound monoclonal antibodies are detected with a goat anti-mouse IgG+IgM (H+ L) F(ab′)²-peroxidase conjugates and the peroxidase substrate diaminobenzidine (1 mg/ml, Sigma Catalog No. D 5637) in 0.1 M sodium acetate buffer pH 5.05 and 0.003% hydrogen peroxide (Sigma cat. No. H1009). The stained slides are counter-stained with hematoxylin and examined under Nikon microscope.

Monoclonal antibody against a CA protein (antigen) is used to test reactivity with various cell lines from different types of tissues. Cells from different established cell lines are removed from the growth surface without using proteases, packed and embedded in OCT compound. The cells are frozen and sectioned, then stained using a standard IHC protocol. The CellArray™ technology is described in WO 01/43869. Normal tissue (human) obtained by surgical resection are frozen and mounted. Cryosections are cut with a Leica 3050 CM mictrotome at thickness of 5 μm and thaw-mounted on vectabound-coated slides. The sections are fixed with ethanol at −20° C. and allowed to air dry overnight at room temperature. PolyMICA™ Detection kit is used to determine binding of a CA-specific monoclonal antibody to normal tissue. Primary monoclonal antibody is used at a final concentration of 1 μg/ml.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A nucleic acid array for detecting a cancer associated (CA) nucleic acid comprising:
at least two nucleic acid probes each comprising at least 10 contiguous nucleotides of at least two sequences selected from the group consisting of the polynucleotide sequences SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46, or its complement.

2. The nucleic acid array according to claim 1, the probes comprising at least 15 contiguous nucleotides.

3. The nucleic acid array according to claim 1, the probes comprising at least 20 contiguous nucleotides.

4. A peptide array comprising at least two isolated polypeptides, each encoded within an open reading frame of a CA sequence selected from the group consisting of the polynucleotide sequences of SEQ ID NOS: 7, 15, 25, 43, 59, 68, 71, 79, 85, 99, 111, 121, 140, 148, 156, 164, 176, 186, 202, 219, 227, 235, 245, 259, 277, 289, 297, 307, 313, 319, 331, 339, 347, 361, 367, 375, 385, 399, and 405 shown in Tables 1-46, or its complement.

5. The peptide array of claim 4, wherein each said polypeptide comprises the amino acid sequence encoded by a polynucleotide selected from the group consisting of SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46.

6. The peptide array of claim 4, wherein each said polypeptide comprises the amino acid sequence encoded by a polypeptide selected from the group consisting of SEQ ID NOS: 9, 11, 17, 19, 21, 27, 45, 47, 61, 70, 73, 81, 87, 89, 91, 93, 95, 101, 113, 115, 123, 142, 144, 150, 152, 158, 160, 166, 168, 170, 172, 178, 180, 182, 188, 190, 192, 194, 196, 198, 204, 206, 208, 210, 221, 223, 229, 231, 237, 239, 247, 249, 261, 263, 265, 267, 269, 279, 281, 291, 299, 309, 315, 321, 323, 325, 333, 335, 341, 343, 349, 351, 353, 355, 357, 363, 369, 371, 377, 379, 381, 387, 389, 391, 393, 401, 407, 409, and 411 shown in Tables 1-46.

7. The peptide array of claim 4, wherein each said polypeptide comprises the amino acid sequence of an epitope of the amino acid sequence of a CA polypeptide selected from the group consisting of SEQ ID NOS: 9, 11, 17, 19, 21, 27, 45, 47, 61, 70, 73, 81, 87, 89, 91, 93, 95, 101, 113, 115, 123, 142, 144, 150, 152, 158, 160, 166, 168, 170, 172, 178, 180, 182, 188, 190, 192, 194, 196, 198, 204, 206, 208, 210, 221, 223, 229, 231, 237, 239, 247, 249, 261, 263, 265, 267, 269, 279, 281, 291, 299, 309, 315, 321, 323, 325, 333, 335, 341, 343, 349, 351, 353, 355, 357, 363, 369, 371, 377, 379, 381, 387, 389, 391, 393, 401, 407, 409, and 411 shown in Tables 1-46.

8. The peptide array of claim 4, wherein said polypeptide is expressed on a cell surface, wherein the CA protein selected from the group consisting of SEQ ID NOS: 8, 10, 18, 20, 22, 28, 30, 42, 44, 46, 48, 54, 62, 68, 70, 72 and 74.

9. A compound that binds to a polypeptide of the peptide array of claim 4.

10. The compound according to claim 9, wherein the compound is chemically synthesized.

11. The compound according to claim 9, wherein the compound is naturally occurring.

12. An isolated antibody or antigen binding fragment thereof, that binds to a polypeptide comprising the amino acid sequence encoded by a polypeptide selected from the group consisting of SEQ ID NOS: 9, 11, 17, 19, 21, 27, 45, 47, 61, 70, 73, 81, 87, 89, 91, 93, 95, 101, 113, 115, 123, 142, 144, 150, 152, 158, 160, 166, 168, 170, 172, 178, 180, 182, 188, 190, 192, 194, 196, 198, 204, 206, 208, 210, 221, 223, 229, 231, 237, 239, 247, 249, 261, 263, 265, 267, 269, 279, 281, 291, 299, 309, 315, 321, 323, 325, 333, 335, 341, 343, 349, 351, 353, 355, 357, 363, 369, 371, 377, 379, 381, 387, 389, 391, 393, 401, 407, 409, and 411 shown in Tables 1-46.

13. The isolated antibody or antigen binding fragment thereof according the claim 12, wherein said antibody or fragment thereof is attached to a solid support.

14. The isolated antibody or antigen binding fragment thereof according the claim 12, wherein said antibody is a monoclonal antibody.

15. The isolated antibody or antigen binding fragment thereof according the claim 12, wherein said antibody is a polyclonal antibody.

16. The isolated antibody or antigen binding fragment thereof according the claim 12, wherein said antibody or fragment thereof further comprises a detectable label.

17. An isolated antibody that binds to a polypeptide, or antigen binding fragment thereof, according to to claim 8, prepared by a method comprising the steps of: (i) immunizing a host animal with a composition comprising said polypeptide, or antigen binding fragment thereof, and (ii) collecting cells from said host expressing antibodies against the antigen or antigen binding fragment thereof.

18. The monoclonal antibody according to claim 14, wherein the monoclonal antibody binds to the extracellular domain of the CA protein.

19. The monoclonal antibody according to claim 14, wherein the monoclonal antibody binds to at least one human cancer cell line.

20. The monoclonal antibody according to claim 14, wherein the monoclonal antibody is prepared by a process comprising:
(a) providing a hybridoma capable of producing the monoclonal antibody; and
(b) culturing the hybridoma under conditions that provide for the production of the monoclonal antibody by the hybridoma.

21. A hybridoma that produces the monoclonal antibody according to claim 14.

22. The antibody according to claim 12, wherein the antibody is a humanized antibody.

23. The antibody according to claim 12, wherein the CAP is expressed on a cancer cell surface but not on a normal cell surface.

24. The antibody according to claim 12, wherein the CAP is differentially expressed on a cancer cell surface relative to a normal cell surface.

25. The antibody according to claim 12, wherein the antibody is linked to a therapeutic agent.

26. The antibody according to claim 14, wherein the antibody is linked to a therapeutic agent.

27. A pharmaceutical composition comprising the antibody according to claim 12 and a pharmaceutically acceptable excipient.

28. A pharmaceutical composition comprising the antibody according to claim 14 and a pharmaceutically acceptable excipient.

29. A pharmaceutical composition comprising the antibody according to claim 15 and a pharmaceutically acceptable excipient.

30. A kit for detecting cancer cells comprising the antibody according to claim 12.

31. A kit for detecting cancer cells comprising the monoclonal antibody according to claim 14.

32. A method for detecting a presence or an absence of cancer cells in an individual, the method comprising:
contacting cells from the individual with the antibody according to claim 12;
and detecting a complex of a CAP from the cancer cells and the antibody,
wherein detection of the complex correlates with the presence of cancer cells in the individual.

33. A kit for diagnosing the presence of cancer in a test sample, said kit comprising at least two polynucleotides that selectively hybridize to at least two CA polynucleotide sequences selected from the group consisting of the polynucleotide sequences SEQ ID NOS: 7, 15, 25, 43, 59, 68, 71, 79, 85, 99, 111, 121, 140, 148, 156, 164, 176, 186, 202, 219, 227, 235, 245, 259, 277, 289, 297, 307, 313, 319, 331, 339, 347, 361, 367, 375, 385, 399, and 405 shown in Tables 1-46, or its complement.

34. A kit for diagnosing the presence of cancer in a test sample, said kit comprising at least two polynucleotides that selectively hybridize to the sequence of at least two polynucleotide sequences selected from the group consisting of the polynucleotide sequences SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46, a fragment thereof, or their complement.

35. An electronic library comprising a polynucleotide, or fragment thereof, comprising at least two CA polynucleotide sequences selected from the group consisting of the polynucleotide sequences of SEQ ID NOS: 7, 15, 25, 43, 59, 68, 71, 79, 85, 99, 111, 121, 140, 148, 156, 164, 176, 186, 202, 219, 227, 235, 245, 259, 277, 289, 297, 307, 313, 319, 331, 339, 347, 361, 367, 375, 385, 399, and 405 shown in Tables 1-46, or its complement.

36. An electronic library comprising a polynucleotide, or fragment thereof, comprising at least two CA polynucleotide sequences selected from the group consisting of the polynucleotide sequences of SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46.

37. An electronic library comprising a polypeptide, or fragment thereof, comprising at least two CA polypeptide sequences selected from the group consisting of the polypeptide sequences of SEQ ID NOS: 9, 11, 17, 19, 21, 27, 45, 47, 61, 70, 73, 81, 87, 89, 91, 93, 95, 101, 113, 115, 123, 142, 144, 150, 152, 158, 160, 166, 168, 170, 172, 178, 180, 182, 188, 190, 192, 194, 196, 198, 204, 206, 208, 210, 221, 223, 229, 231, 237, 239, 247, 249, 261, 263, 265, 267, 269, 279, 281, 291, 299, 309, 315, 321, 323, 325, 333, 335, 341, 343, 349, 351, 353, 355, 357, 363, 369, 371, 377, 379, 381, 387, 389, 391, 393, 401, 407, 409, and 411 shown in Tables 1-46.

38. A method of screening for anticancer activity comprising:
(a) providing a cell that expresses a cancer associated (CA) gene encoded by a nucleic acid sequence selected from the group consisting of the sequences SEQ ID NOS: 7, 15, 25, 43, 59, 68, 71, 79, 85, 99, 111, 121, 140, 148, 156, 164, 176, 186, 202, 219, 227, 235, 245, 259, 277, 289, 297, 307, 313, 319, 331, 339, 347, 361, 367, 375, 385, 399, and 405 shown in Tables 1-46, or fragment thereof;
(b) contacting a tissue sample derived from a cancer cell with an anticancer drug candidate; and
(c) monitoring an, effect of the anticancer drug candidate on an expression of the CA polynucleotide in the tissue sample.

39. The method of screening for anticancer activity according to claim 38, wherein the CA gene comprises at least one nucleic acid sequence selected from the group consisting of the sequences SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46.

40. The method of screening for anticancer activity according to claim 38, further comprising:
(d) comparing the level of expression in the absence of said drug candidate to the level of expression in the presence of the drug candidate.

41. The method of screening for anticancer activity according to claim 39, wherein the drug candidate is an inhibitor of transcription and further wherein the nucleic acid sequence is selected from the group consisting of SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46.

42. A method for detecting cancer associated with expression of a polypeptide in a test cell sample, comprising the steps of:
(i) detecting a level of expression of at least one polypeptide selected from the group consisting of SEQ ID NOS: 9, 11, 17, 19, 21, 27, 45, 47, 61, 70, 73, 81, 87, 89, 91, 93, 95, 101, 113, 115, 123, 142, 144, 150, 152, 158, 160, 166, 168, 170, 172, 178, 180, 182, 188, 190, 192, 194, 196, 198, 204, 206, 208, 210, 221, 223, 229, 231, 237, 239, 247, 249, 261, 263, 265, 267, 269, 279, 281, 291, 299, 309, 315, 321, 323, 325, 333, 335, 341, 343, 349, 351, 353, 355, 357, 363, 369, 371, 377, 379, 381, 387, 389, 391, 393, 401, 407, 409, and 411 shown in Tables 1-46, or a fragment thereof; and
(ii) comparing the level of expression of the polypeptide in the test sample with a level of expression of polypeptide in a normal cell sample, wherein an altered level of expression of the polypeptide in the test cell sample relative to the level of polypeptide expression in the normal cell sample is indicative of the presence of cancer in the test cell sample.

43. A method for detecting cancer associated with expression of a polypeptide in a test cell sample, comprising the steps of:
(i) detecting a level of activity of at least one polypeptide selected from the group consisting of SEQ ID NOS: 9, 11, 17, 19, 21, 27, 45, 47, 61, 70, 73, 81, 87, 89, 91, 93, 95, 101, 113, 115, 123, 142, 144, 150, 152, 158, 160, 166, 168, 170, 172, 178, 180, 182, 188, 190, 192, 194, 196, 198, 204, 206, 208, 210, 221, 223, 229, 231, 237, 239, 247, 249, 261, 263, 265, 267, 269, 279, 281, 291, 299, 309, 315, 321, 323, 325, 333, 335, 341, 343, 349, 351, 353, 355, 357, 363, 369, 371, 377, 379, 381, 387, 389, 391, 393, 401, 407, 409, and 411 shown in Tables 1-46, or a fragment thereof, wherein said activity corresponds to at least one activity for the polypeptide listed in Tables 1-46; and
(ii) comparing the level of activity of the polypeptide in the test sample with a level of activity of polypeptide in a normal cell sample, wherein an altered level of activity of the polypeptide in the test cell sample relative to the level of polypeptide activity in the normal cell sample is indicative of the presence of cancer in the test cell sample.

44. A method for detecting cancer associated with the presence of an antibody in a test serum sample, comprising the steps of:
(i) detecting a level of an antibody against an antigenic polypeptide selected from the group consisting of SEQ ID NOS: 9, 11, 17, 19, 21, 27, 45, 47, 61, 70, 73, 81, 87, 89, 91, 93, 95, 101, 113, 115, 123, 142, 144, 150, 152, 158, 160, 166, 168, 170, 172, 178, 180, 182, 188, 190, 192, 194, 196, 198, 204, 206, 208, 210, 221, 223, 229, 231, 237, 239, 247, 249, 261, 263, 265, 267, 269, 279, 281, 291, 299, 309, 315, 321, 323, 325, 333, 335, 341, 343, 349, 351, 353, 355, 357, 363, 369, 371, 377, 379, 381, 387, 389, 391, 393, 401, 407, 409, and 411 shown in Tables 1-46, or antigenic fragment thereof; and
(ii) comparing said level of said antibody in the test sample with a level of said antibody in the control sample, wherein an altered level of antibody in said test sample relative to the level of antibody in the control sample is indicative of the presence of cancer in the test serum sample.

45. A method for screening for a bioactive agent capable of modulating the activity of a CA protein (CAP), wherein said CAP is encoded by a nucleic acid comprising a nucleic acid sequence selected from the group consisting of the polynucleotide sequences SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46, said method comprising:
a) combining said CAP and a candidate bioactive agent; and
b) determining the effect of the candidate agent on the bioactivity of said CAP.

46. The method of screening for the bioactive agent according to claim 45, wherein the bioactive agent affects the expression of the CA protein (CAP).

47. The method of screening for the bioactive agent according to claim 45, wherein the bioactive agent affects the activity of the CA protein (CAP), wherein such activity is selected from the activities listed in Tables 1-46.

48. The method of screening for the bioactive agent according to claim 45, wherein the bioactive agent is a modulator of an activity selected from the group consisting of: G-protein coupled receptor, nucleotide binding, nucleic acid binding, calcium binding protein, apoptosis, cell cycle regulator, chromatin protein transcription factor, transport protein, signal transduction, nuclear receptor, growth factor, protein kinase, protein phosphorylase, peptidase, electron transport, thioredoxin, semaphorin, signaling, and motility as shown in Tables 1-46.

49. A method for diagnosing cancer comprising:
a) determining the expression of one or more genes comprising a nucleic acid sequence selected from the group consisting of the human genomic and mRNA sequences outlined in Tables 1-46, in a first tissue type of a first individual; and
b) comparing said expression of said gene(s) from a second normal tissue type from said first individual or a second unaffected individual;
wherein a difference in said expression indicates that the first individual has cancer.

50. A method for treating cancers comprising administering to a patient an inhibitor of a CA protein (CAP), wherein said CAP is encoded by a nucleic acid comprising a nucleic acid sequence selected from the group consisting of the human nucleic acid sequences in Tables 1-46.

51. The method for treating cancers according to claim 50, wherein the inhibitor of a CA protein (CAP) binds to the CA protein.

52. A method for inhibiting expression of a cancer associated (CA) gene in a cell comprising:
contacting a cell expressing a CA gene with a double stranded RNA comprising a sequence capable of hybridizing to a cancer associated (CA) mRNA corresponding to the polynucleotide sequences of SEQ ID NOS: 8, 10, 16, 18, 20, 26, 44, 46, 60, 69, 72, 80, 86, 88, 90, 92, 94, 100, 112, 114, 122, 141, 143, 149, 151, 157, 159, 165, 167, 169, 171, 177, 179, 181, 187, 189, 191, 193, 195, 197, 203, 205, 207, 209, 220, 222, 228, 230, 236, 238, 246, 248, 260, 262, 264, 266, 268, 278, 280, 290, 298, 308, 314, 320, 322, 324, 332, 334, 340, 342, 348, 350, 352, 354, 356, 362, 368, 370, 376, 378, 380, 386, 388, 390, 392, 400, 406, 408, and 410 shown in Tables 1-46, in an amount sufficient to elicit RNA interference; and
inhibiting expression of the CA gene in the cell.

53. The method of claim 52, wherein the double stranded RNA is provided by introducing a short interfering RNA (siRNA) into the cell by a method selected from the group consisting of transfection, electroporation, and microinjection.

54. The method of claim 52, wherein the double stranded RNA is provided by introducing a short interfering RNA (siRNA) into the cell by an expression vector.
